(19) United States
(12) Patent Application Publication
(10) Pub. No.: US 2020/0235297 A1
(43) Pub. Date: Jul. 23, 2020

(54) ORGANIC ELECTROLUMINESCENCE DEVICE AND MONOAMINE COMPOUND FOR ORGANIC ELECTROLUMINESCENCE DEVICE

(71) Applicant: Samsung Display Co., Ltd., Yongin-Si (KR)

(72) Inventors: Hideo MIYAKE, Yokohama (JP); Masatsugu UENO, Yokohama (JP); Xiulan JIN, Yokohama (JP); Ichinori TAKADA, Yokohama (JP); Takuya UNO, Yokohama (JP); Hiroaki ITOI, Yokohama (JP)

(21) Appl. No.: 16/842,556

(22) Filed: Apr. 7, 2020

Related U.S. Application Data
(63) Continuation-in-part of application No. 16/256,225, filed on Jan. 24, 2019.

(30) Foreign Application Priority Data
Jan. 26, 2018 (KR) 10-2018-0009993
Nov. 23, 2018 (KR) 10-2018-0146236

(51) Int. Cl. H01L 51/00; C07C 211/54; C09K 11/06; C07D 209/88; C07F 7/08; C07C 211/61; C07D 333/76; C07C 211/56; C07D 307/91

(52) U.S. Cl. CPC H01L 51/006; C07C 211/54; C09K 11/06; C07D 209/88; H01L 51/0061; H01L 51/5056; H01L 51/0094; C07C 211/61; C07D 333/76; C07C 211/56; C07D 307/91; C07F 7/081

(57) ABSTRACT

An organic electroluminescence device includes a first electrode, a hole transport region on the first electrode, an emission layer on the hole transport region, an electron transport region on the emission layer, and a second electrode on the electron transport region. The hole transport region includes a monoamine compound represented by the following Formula 1:

### CROSS-REFERENCE TO RELATED APPLICATION

The present application is a continuation-in-part of U.S. patent application Ser. No. 16/256,225, filed on Jan. 24, 2019, which claims priority to and the benefit of Korean Patent Application No. 10-2018-0009993, filed on Jan. 26, 2018, and Korean Patent Application No. 10-2018-0146236, filed on Nov. 23, 2018, in the Korean Intellectual Property Office, and each entitled: “Organic Electroluminescence Device and Monoamine Compound for Organic Electroluminescence Device,” the contents of all of which are incorporated by reference herein in their entireties.

### BACKGROUND

### 1. Field

Embodiments relate to an organic electroluminescence device and a monoamine compound for an organic electroluminescence device.

### 2. Description of the Related Art

Development on an organic electroluminescence display as an image display is being actively conducted. An organic electroluminescence display is different from a liquid crystal display and is so called a self-luminescent display which accomplishes display by recombining holes and electrons injected from a first electrode and a second electrode in an emission layer and emitting light from a luminescent material which includes an organic compound in the emission layer.

### SUMMARY

Embodiments are directed to an organic electroluminescence device including a first electrode, a hole transport region on the first electrode, an emission layer on the hole transport region, an electron transport region on the emission layer and a second electrode on the electron transport region, in which the hole transport region includes a monoamine compound represented by the following Formula 1.

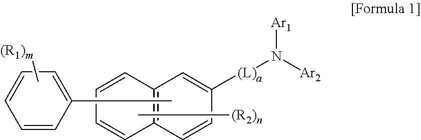

In Formula 1, Ar₁ and Ar₂ may each independently be a substituted or unsubstituted alkyl group having 1 to 10 carbon atoms, a substituted or unsubstituted cycloalkyl group having 3 to 20 ring carbon atoms, a substituted or unsubstituted aryl group having 6 to 30 ring carbon atoms, or a substituted or unsubstituted heteroaryl group having 2 to 30 ring carbon atoms, L may be a substituted or unsubstituted arylene group having 6 to 30 ring carbon atoms, or a substituted or unsubstituted heteroarylene group having 2 to 30 ring carbon atoms, R₁ may be a hydrogen atom, a deuterium atom, a halogen atom, a substituted or unsubstituted alkyl group having 1 to 20 carbon atoms, a substituted or unsubstituted cycloalkyl group having 3 to 20 ring carbon atoms, or a substituted or unsubstituted aryl group having 6 to 30 ring carbon atoms, R₂ may be a hydrogen atom, a deuterium atom, a halogen atom, a substituted or unsubstituted alkyl group having 1 to 20 carbon atoms, or a substituted or unsubstituted cycloalkyl group having 3 to 20 ring carbon atoms, a may be an integer of 0 to 3, m may be an integer of 0 to 1, n may be an integer of 0 to 6, and when any one of Ar₁ and Ar₂ is 3-dibenzofuranyl, the other may not be 9-phenanthryl.

The hole transport region may have a plurality of layers, and a layer of the plurality of layers contacting the emission layer may include the monoamine compound according to an example embodiment.

The hole transport region may include a hole injection layer on the first electrode, a hole transport layer on the hole injection layer, and an electron blocking layer on the hole transport layer, and the electron blocking layer may include the monoamine compound according to an example embodiment.

The electron transport region may include a hole blocking layer on the emission layer, an electron transport layer on the hole blocking layer, and an electron injection layer on the electron transport layer.

Formula 1 may be represented by any one of the following Formulae 2 to 8.

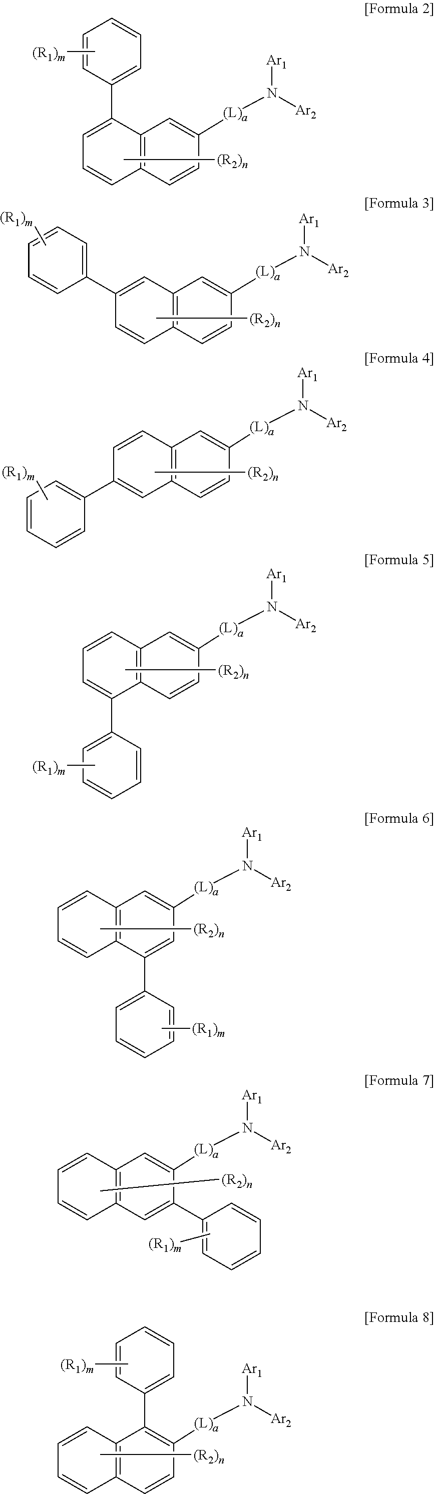

In Formulae 2 to 8, Ar₁, Ar₂, L, R₁, R₂, a, m, and n are the same as defined in Formula 1.

L may be a substituted or unsubstituted arylene group having 6 to 12 ring carbon atoms.

L may be a substituted or unsubstituted phenylene group.

Ar₁ and Ar₂ may be each independently a substituted or unsubstituted aryl group having 6 to 12 ring carbon atoms.

Ar₁ and Ar₂ may be each independently a substituted or unsubstituted phenyl group, a substituted or unsubstituted biphenyl group, a substituted or unsubstituted naphthyl group, or a substituted or unsubstituted fluorenyl group.

Ar₁ and Ar₂ may be each independently a substituted or unsubstituted heteroaryl group having 5 to 12 ring carbon atoms.

Ar₁ and Ar₂ may be each independently a substituted or unsubstituted dibenzofuran group, a substituted or unsubstituted dibenzothiophene group, or a substituted or unsubstituted carbazole group.

At least one of Ar₁ and Ar₂ may be a substituted or unsubstituted 4-dibenzothiophene group.

Formula 1 may be represented by the following Formula 9.

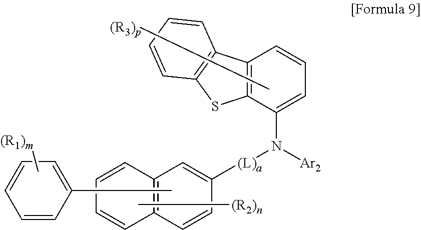

In Formula 9, R₃ may be a hydrogen atom, a deuterium atom, a halogen atom, a substituted or unsubstituted alkyl group having 1 to 20 carbon atoms, a substituted or unsubstituted cycloalkyl group having 3 to 20 ring carbon atoms, or a substituted or unsubstituted aryl group having 6 to 30 ring carbon atoms. p may be an integer of 0 to 7.

In Formula 9, Ar₂, L, R₁, R₂, a, m, and n are the same as defined in Formula 1.

Embodiments are also directed to a monoamine compound represented by the above Formula 1.

### BRIEF DESCRIPTION OF THE DRAWINGS

Features will become apparent to those of skill in the art by describing in detail example embodiments with reference to the attached drawings in which:

FIG. 1 illustrates a schematic cross-sectional view of an organic electroluminescence device according to an example embodiment;

FIG. 2 illustrates a schematic cross-sectional view of an organic electroluminescence device according to an example embodiment; and

FIG. 3 illustrates a schematic cross-sectional view of an organic electroluminescence device according to an example embodiment.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey example implementations to those skilled in the art. In the drawing figures, the dimensions of layers and regions may be exaggerated for clarity of illustration. Like reference numerals refer to like elements throughout.

It will be understood that, although the terms first, second, etc. may be used herein to describe various elements, these elements should not be limited by these terms. These terms are only used to distinguish one element from another element. For example, a first element discussed below could be termed a second element, and similarly, a second element could be termed a first element. As used herein, the singular forms are intended to include the plural forms as well, unless the context clearly indicates otherwise.

It will be further understood that the terms “comprise” or “have,” when used in this specification, specify the presence of stated features, numerals, steps, operations, elements, parts, or a combination thereof, but do not preclude the presence or addition of one or more other features, numerals, steps, operations, elements, parts, or a combination thereof. It will also be understood that when a layer, a film, a region, a plate, etc. is referred to as being “on” another part, it can be “directly on” the other part, or intervening layers may also be present. On the other hand, when a layer, a film, a region, a plate, etc. is referred to as being “under” another part, it can be “directly under” the other part, or intervening layers may also be present.

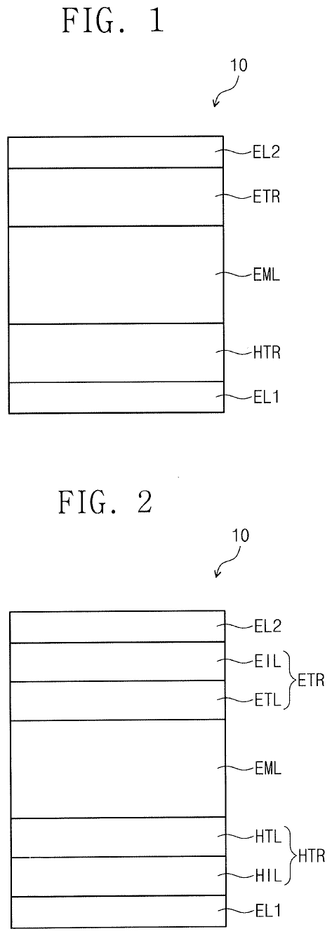

First, an organic electroluminescence device according to an example embodiment will be explained referring to FIGS. 1 to 3.

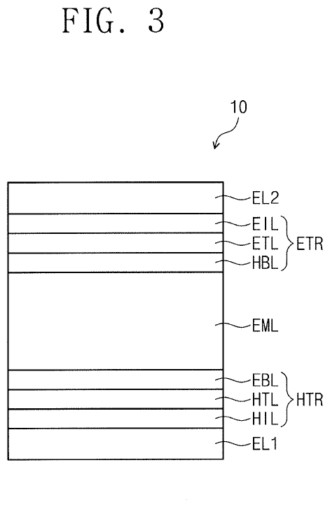

FIG. 1 is a schematic cross-sectional view illustrating an organic electroluminescence device according to an example embodiment. FIG. 2 is a schematic cross-sectional view illustrating an organic electroluminescence device according to an example embodiment. FIG. 3 is a schematic cross-sectional view illustrating an organic electroluminescence device according to an example embodiment.

Referring to FIGS. 1 to 3, an organic electroluminescence device 10 according to an example embodiment includes a first electrode ELL a hole transport region HTR, an emission layer EML, an electron transport region ETR, and a second electrode EL2.

The hole transport region HTR includes the monoamine compound according to an example embodiment. Hereinafter, the monoamine compound according to an example embodiment will be specifically explained, and then each layer of the organic electroluminescence device 10 will be explained.

In the present disclosure, “substituted or unsubstituted” may mean unsubstituted or substituted with at least one substituent selected from the group of deuterium, halogen, cyano, nitro, silyl, boron, phosphine, alkyl, alkenyl, aryl and heterocyclic group. In addition, each of the substituent described above may be substituted or unsubstituted. For example, biphenyl may be interpreted as aryl, or phenyl substituted with phenyl.

In the present disclosure, examples of a halogen atom are a fluorine atom, a chlorine atom, a bromine atom, and an iodine atom.

In the present disclosure, the alkyl group may have a linear, branched or cyclic form. The carbon number of the alkyl group may be 1 to 30, 1 to 20, 1 to 10, or 1 to 4. Examples of the alkyl group may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldodecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyl eicosyl, 2-butyl eicosyl, 2-hexyl eicosyl, 2-octyl eicosyl, n-heneicosyl, n-docosyl, n-tricosyl, n-tetracosyl, n-pentacosyl, n-hexacosyl, n-heptacosyl, n-octacosyl, n-nonacosyl, n-triacontyl, etc.

In the present disclosure, the aryl group means any functional group or substituent derived from an aromatic hydrocarbon ring. The aryl group may be monocyclic aryl or polycyclic aryl. The carbon number of the aryl group for forming a ring may be 6 to 30, 6 to 20, or 6 to 12. Examples of the aryl group may include phenyl, naphthyl, fluorenyl, anthracenyl, phenanthryl, biphenyl, terphenyl, quaterphenyl, quinqphenyl, sexiphenyl, biphenylene, triphenylene, pyrenyl, benzofluoranthenyl, chrysenyl, etc.

In the present disclosure, the fluorenyl group may be substituted, and two substituents may be combined with each other to form a spiro structure. Examples of the substituted fluorenyl group may include the following groups:

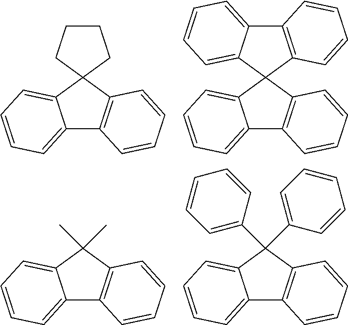

In the present disclosure, the heteroaryl group may be heteroaryl including at least one of 0, N, P, Si, or S as a heteroatom. When the heteroaryl group includes two heteroatoms, the two heteroatoms may be the same or different from each other. The carbon number of the heteroaryl group for forming a ring may be 2 to 30, or 5 to 12. The heteroaryl group may be monocyclic heteroaryl or polycyclic heteroaryl. Polycyclic heteroaryl may have bicyclic or tricyclic structure, for example. Examples of the heteroaryl group may include thiophene, furan, pyrrole, imidazole, thiazole, oxazole, oxadiazole, triazole, pyridine, bipyridine, pyrimidine, triazine, triazole, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-aryl carbazole, N-heteroaryl carbazole, N-alkyl carbazole, benzoxazole, benzoimidazole, benzothiazole, benzocarbazole, benzothiophene, dibenzothiophene, thienothiophene, benzofuran, phenanthroline, thiazole, isoxazole, oxadiazole, thiadiazole, phenothiazine, dibenzosilole, dibenzofuran, etc.

In the present disclosure, the silyl group may include alkyl silyl and aryl silyl. Examples of the silyl group may include trimethylsilyl, triethylsilyl, t-butyl dimethylsilyl, vinyl dimethylsilyl, propyl dimethylsilyl, triphenylsilyl, diphenylsilyl, phenylsilyl, etc.

In the present disclosure, the boron group may include alkyl boron and aryl boron. Examples of the boron group may include trimethyl boron, triethyl boron, t-butyl dimethyl boron, triphenyl boron, diphenyl boron, phenyl boron, etc.

In the present disclosure, the alkenyl group may be linear or branched. The carbon number is not specifically limited, and may be 2 to 30, 2 to 20, or 2 to 10. Examples of the alkenyl group may include vinyl, 1-butenyl, 1-pentenyl, 1,3-butadienyl aryl, styrenyl, styrylvinyl, etc.

The above explanation on the aryl group may be applied to the arylene group, except that the arylene group is divalent.

The above explanation on the heteroaryl group may be applied to the heteroarylene group, except that the heteroarylene group is divalent.

A monoamine compound according to an example embodiment is represented by the following Formula 1.

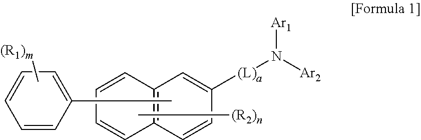

According to the present example embodiment, in Formula 1, Ar₁ and Ar₂ may each independently be a substituted or unsubstituted alkyl group having 1 to 10 carbon atoms, a substituted or unsubstituted cycloalkyl group having 3 to 20 ring carbon atoms, a substituted or unsubstituted aryl group having 6 to 30 ring carbon atoms, or a substituted or unsubstituted heteroaryl group having 2 to 30 ring carbon atoms.

In Formula 1, L may be a substituted or unsubstituted arylene group having 6 to 30 ring carbon atoms, or a substituted or unsubstituted heteroarylene group having 2 to 30 ring carbon atoms.

In Formula 1, R₁ may be a hydrogen atom, a deuterium atom, a halogen atom, a substituted or unsubstituted alkyl group having 1 to 20 carbon atoms, a substituted or unsubstituted cycloalkyl group having 3 to 20 ring carbon atoms, or a substituted or unsubstituted aryl group having 6 to 30 ring carbon atoms.

In Formula 1, R₂ may be a hydrogen atom, a deuterium atom, a halogen atom, a substituted or unsubstituted alkyl group having 1 to 20 carbon atoms, or a substituted or unsubstituted cycloalkyl group having 3 to 20 ring carbon atoms.

In an example embodiment, R₂ may be neither an aryl group nor a heteroaryl group. In a compound of Formula 1 in which R₂ is an aryl group or a heteroaryl group, the naphthalene structure may have a largely distributed HOMO (highest occupied molecular orbital) energy level and the amine group may not maintain the property of extending device life due to the relatively decreased electron density, thereby decreasing life of the organic electroluminescence device including the compound. When R₂ is referred to as being neither an aryl group nor a heteroaryl group, it may include both the case where R₂ is neither an aryl group nor a heteroaryl group and the case where R₂ is substituted with neither an aryl group nor a heteroaryl group.

In Formula 1, a may be an integer of 0 to 3. In case a is an integer of 2 or more, a plurality of L may be the same or different from each other.

In Formula 1, m may be an integer of 0 to 1.

In Formula 1, n may be an integer of 0 to 6. In case n is an integer of 2 or more, a plurality of R₂ may be the same or different from each other.

In Formula 1, in case any one of Ar₁ and Ar₂ is 3-dibenzofuranyl, the other may not be 9-phenanthryl. For example, when Ar₁ is 3-dibenzofuranyl, Ar₂ is not 9-phenanthryl, and when Ar₂ is 3-dibenzofuranyl, Ar₁ is not 9-phenanthryl. Furthermore, the compound of Formula 1, where the nitrogen atom is substituted with both of 3-dibenzofuranyl and 9-phenanthryl, is excluded. A compound of Formula 1 in which the nitrogen atom is substituted with both of 3-dibenzofuranyl and 9-phenanthryl may have a strong molecular stacking and increased deposition temperature, which may result in thermal decomposition and thereby degrade the quality of organic electroluminescence device including the compound.

In an embodiment, Formula 1 may be represented by any one of the following Formulae 2 to 8.

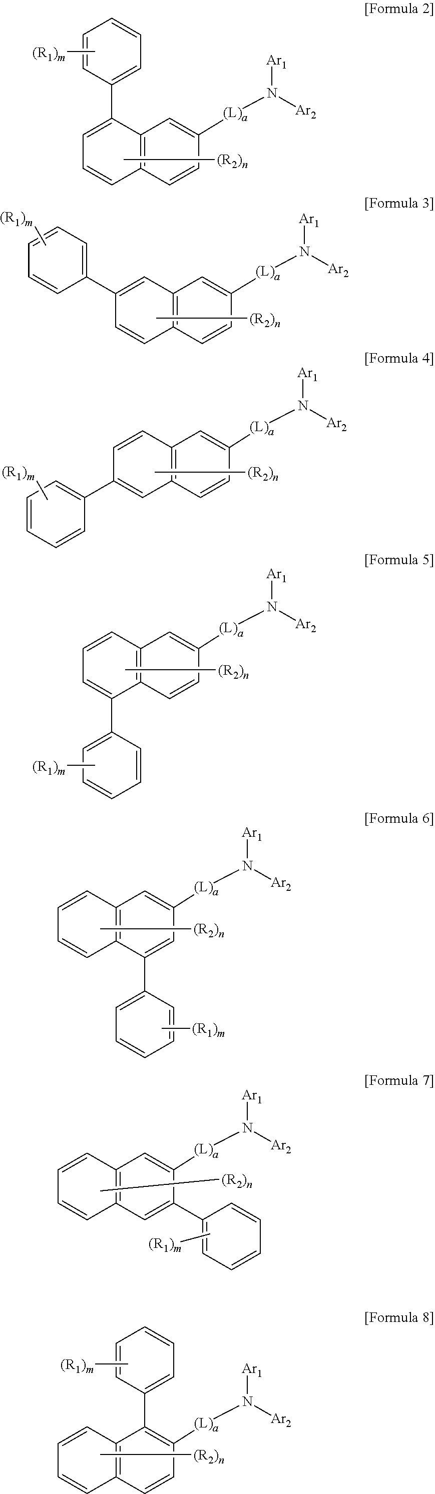

In Formulae 2 to 8, Ar₁, Ar₂, L, R₁, R₂, a, m, and n are the same as defined in Formula 1.

In Formula 1, m may be 1, and L may be a substituted or unsubstituted arylene group having 6 to 12 ring carbon atoms. For example, L may be a substituted or unsubstituted phenylene group.

In Formula 1, Ar₁ and Ar₂ may be each independently a substituted or unsubstituted aryl group having 6 to 12 ring carbon atoms. For example, Ar₁ and Ar₂ may be each independently a substituted or unsubstituted phenyl group, a substituted or unsubstituted biphenyl group, a substituted or unsubstituted naphthyl group, or a substituted or unsubstituted fluorenyl group.

In Formula 1, Ar₁ and Ar₂ may be each independently a substituted or unsubstituted heteroaryl group having 5 to 12 ring carbon atoms. For example, Ar₁ and Ar₂ may be each independently a substituted or unsubstituted dibenzofuran group, a substituted or unsubstituted dibenzothiophene group, or a substituted or unsubstituted carbazole group. At least one of Ar₁ and Ar₂ may be a substituted or unsubstituted 4-dibenzothiophene group. For example, Ar₁ and Ar₂ may all be unsubstituted 4-dibenzothiophene groups. Ar₁ may be an unsubstituted 4-dibenzothiophene group, and Ar₂ may be an unsubstituted m-terphenyl group. Ar₁ may be an unsubstituted 4-dibenzothiophene group, and Ar₂ may be an unsubstituted 4-naphthylphenyl group.

In an embodiment, Formula 1 may be represented by the following Formula 9.

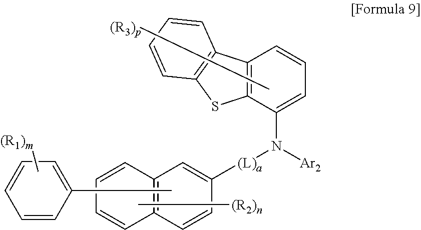

In Formula 9, R₃ may be a hydrogen atom, a deuterium atom, a halogen atom, a substituted or unsubstituted alkyl group having 1 to 20 carbon atoms, a substituted or unsubstituted cycloalkyl group having 3 to 20 ring carbon atoms, or a substituted or unsubstituted aryl group having 6 to 30 ring carbon atoms. For example, R₃ may be a hydrogen atom, or a deuterium atom.

p may be an integer of 0 to 7. In case p is an integer of 2 or more, a plurality of R₃ may be the same or different from each other.

In Formula 1, R₂ may be a hydrogen atom or a deuterium atom.

The monoamine compound represented by Formula 1 according to an example embodiment may be any one selected from the group of compounds represented in the following Compound Groups 1 to 7.

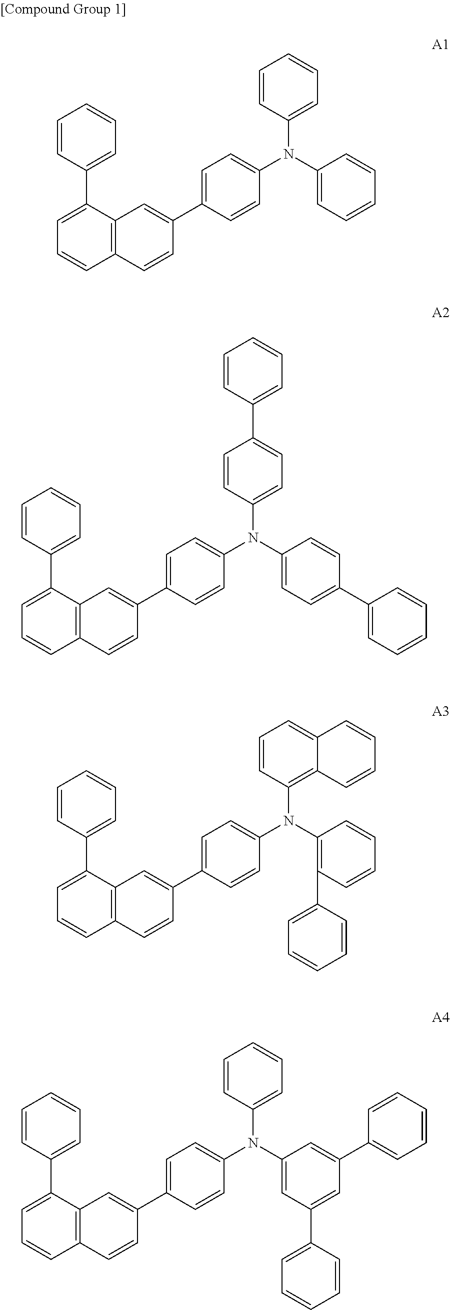 [SMILES: c1ccc(-c2cccc3ccc(-c4ccc(N(c5ccccc5)c5ccccc5)cc4)cc23)cc1 ; c1ccc(-c2ccc(N(c3ccc(-c4ccccc4)cc3)c3ccc(-c4ccc5cccc(-c6ccccc6)c5c4)cc3)cc2)cc1 ; c1ccc(-c2ccccc2N(c2ccc(-c3ccc4cccc(-c5ccccc5)c4c3)cc2)c2cccc3ccccc23)cc1 ; c1ccc(-c2cc(-c3ccccc3)cc(N(c3ccccc3)c3ccc(-c4ccc5cccc(-c6ccccc6)c5c4)cc3)c2)cc1]

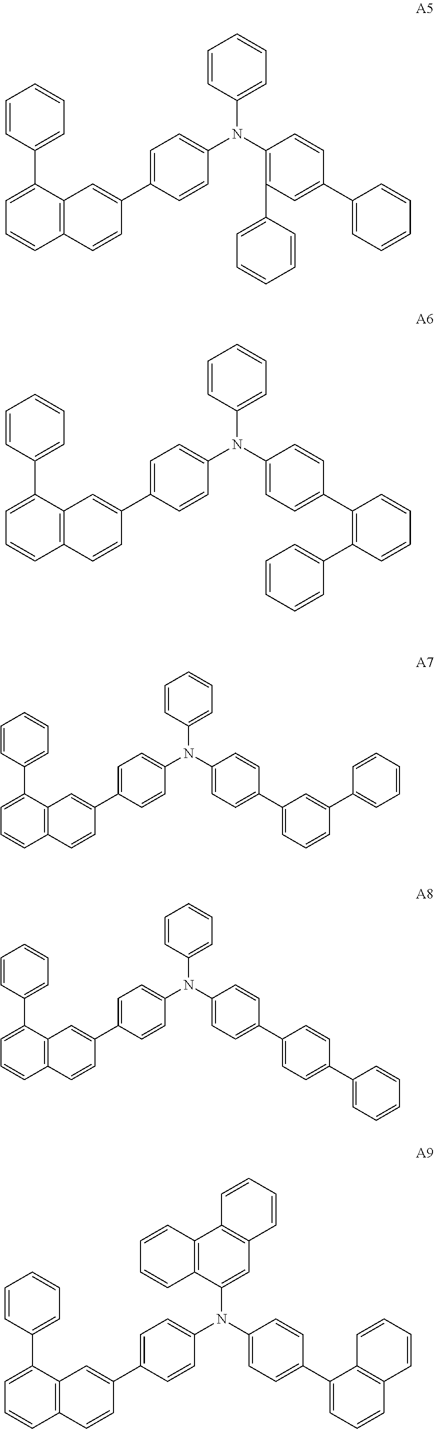 [SMILES: c1ccc(-c2ccc(N(c3ccccc3)c3ccc(-c4ccc5cccc(-c6ccccc6)c5c4)cc3)c(-c3ccccc3)c2)cc1 ; c1ccc(-c2ccccc2-c2ccc(N(c3ccccc3)c3ccc(-c4ccc5cccc(-c6ccccc6)c5c4)cc3)cc2)cc1 ; c1ccc(-c2cccc(-c3ccc(N(c4ccccc4)c4ccc(-c5ccc6cccc(-c7ccccc7)c6c5)cc4)cc3)c2)cc1 ; c1ccc(-c2ccc(-c3ccc(N(c4ccccc4)c4ccc(-c5ccc6cccc(-c7ccccc7)c6c5)cc4)cc3)cc2)cc1 ; c1ccc(-c2cccc3ccc(-c4ccc(N(c5ccc(-c6cccc7ccccc67)cc5)c5cc6ccccc6c6ccccc56)cc4)cc23)cc1]

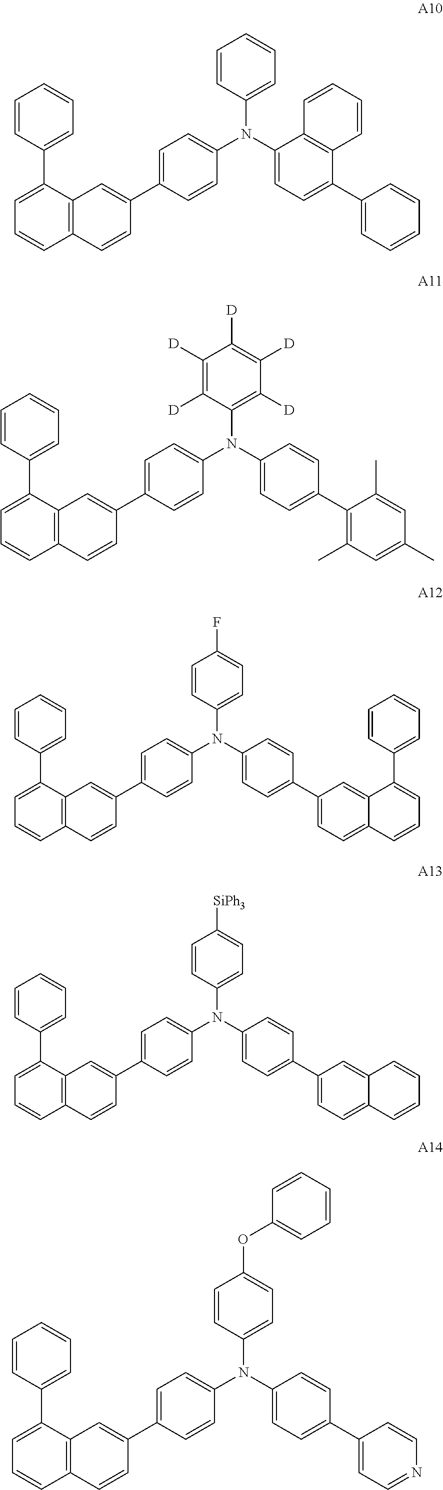

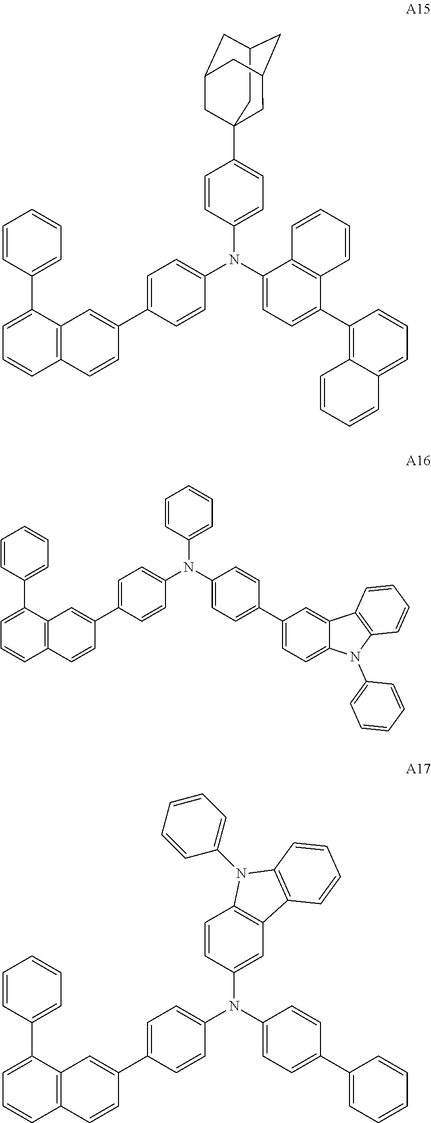 [SMILES: c1ccc(-c2cccc3ccc(-c4ccc(N(c5ccc(C67CC8CC(CC(C8)C6)C7)cc5)c5ccc(-c6cccc7ccccc67)c6ccccc56)cc4)cc23)cc1 ; c1ccc(-c2cccc3ccc(-c4ccc(N(c5ccccc5)c5ccc(-c6ccc7c(c6)c6ccccc6n7-c6ccccc6)cc5)cc4)cc23)cc1 ; c1ccc(-c2ccc(N(c3ccc(-c4ccc5cccc(-c6ccccc6)c5c4)cc3)c3ccc4c(c3)c3ccccc3n4-c3ccccc3)cc2)cc1]

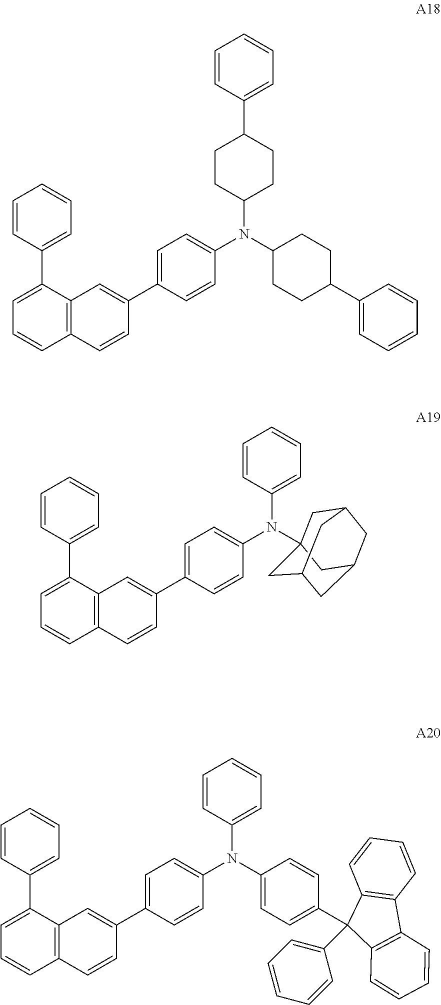 [SMILES: c1ccc(-c2cccc3ccc(-c4ccc(N(C5CCC(c6ccccc6)CC5)C5CCC(c6ccccc6)CC5)cc4)cc23)cc1 ; c1ccc(-c2cccc3ccc(-c4ccc(N(c5ccccc5)C56CC7CC(CC(C7)C5)C6)cc4)cc23)cc1 ; c1ccc(-c2cccc3ccc(-c4ccc(N(c5ccccc5)c5ccc(C6(c7ccccc7)c7ccccc7-c7ccccc76)cc5)cc4)cc23)cc1]

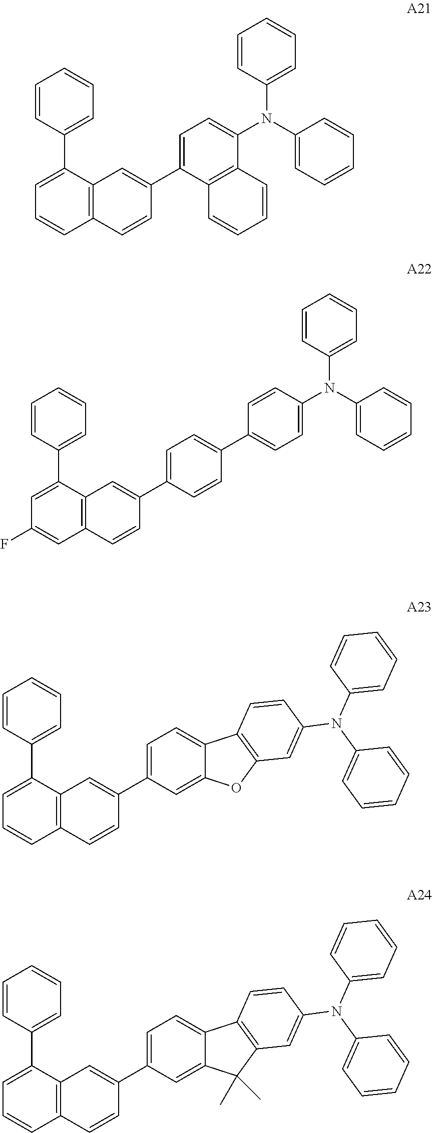 [SMILES: c1ccc(-c2cccc3ccc(-c4ccc(N(c5ccccc5)c5ccccc5)c5ccccc45)cc23)cc1 ; Fc1cc(-c2ccccc2)c2cc(-c3ccc(-c4ccc(N(c5ccccc5)c5ccccc5)cc4)cc3)ccc2c1 ; c1ccc(-c2cccc3ccc(-c4ccc5c(c4)oc4cc(N(c6ccccc6)c6ccccc6)ccc45)cc23)cc1 ; CC1(C)c2cc(-c3ccc4cccc(-c5ccccc5)c4c3)ccc2-c2ccc(N(c3ccccc3)c3ccccc3)cc21]

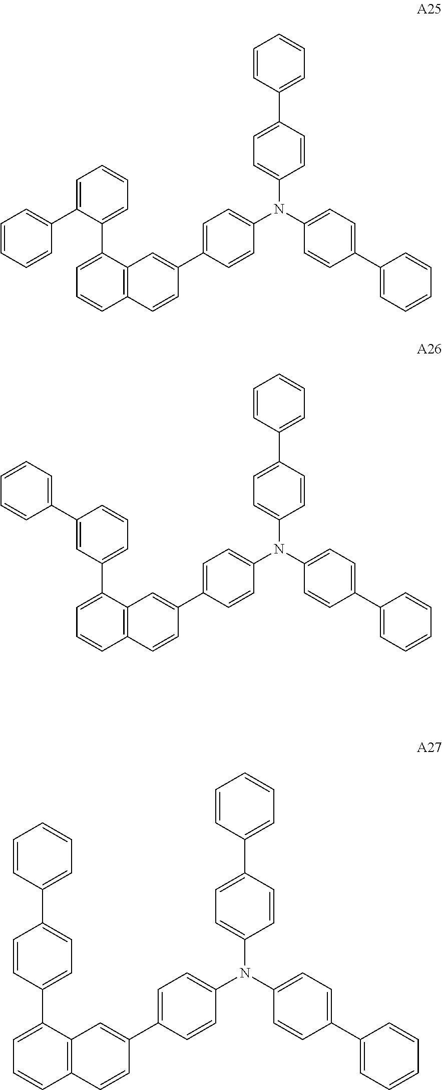 [SMILES: c1ccc(-c2ccc(N(c3ccc(-c4ccccc4)cc3)c3ccc(-c4ccc5cccc(-c6ccccc6-c6ccccc6)c5c4)cc3)cc2)cc1 ; c1ccc(-c2ccc(N(c3ccc(-c4ccccc4)cc3)c3ccc(-c4ccc5cccc(-c6cccc(-c7ccccc7)c6)c5c4)cc3)cc2)cc1 ; c1ccc(-c2ccc(-c3cccc4ccc(-c5ccc(N(c6ccc(-c7ccccc7)cc6)c6ccc(-c7ccccc7)cc6)cc5)cc34)cc2)cc1]

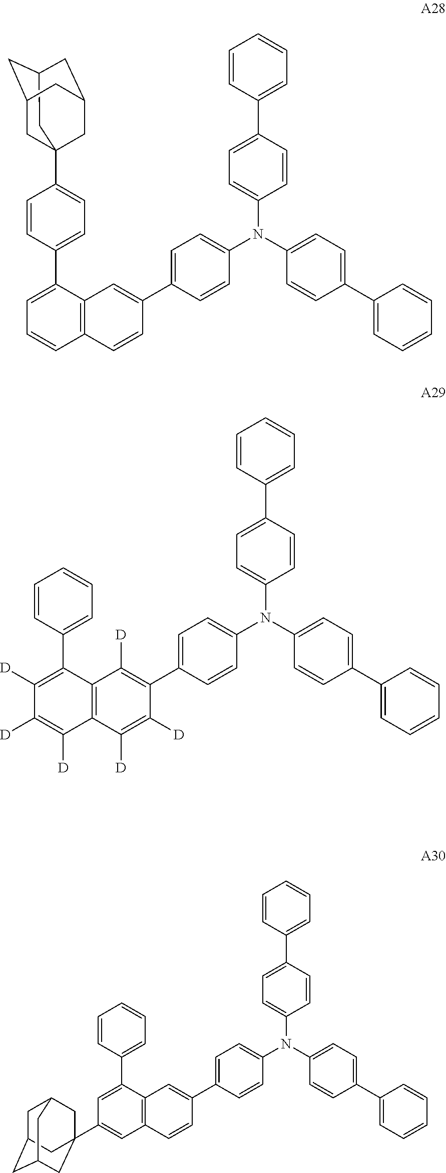 [SMILES: c1ccc(-c2ccc(N(c3ccc(-c4ccccc4)cc3)c3ccc(-c4ccc5cccc(-c6ccc(C78CC9CC(CC(C9)C7)C8)cc6)c5c4)cc3)cc2)cc1 ; [2H]c1c([2H])c(-c2ccccc2)c2c([2H])c(-c3ccc(N(c4ccc(-c5ccccc5)cc4)c4ccc(-c5ccccc5)cc4)cc3)c([2H])c([2H])c2c1[2H] ; c1ccc(-c2ccc(N(c3ccc(-c4ccccc4)cc3)c3ccc(-c4ccc5cc(C67CC8CC(CC(C8)C6)C7)cc(-c6ccccc6)c5c4)cc3)cc2)cc1]

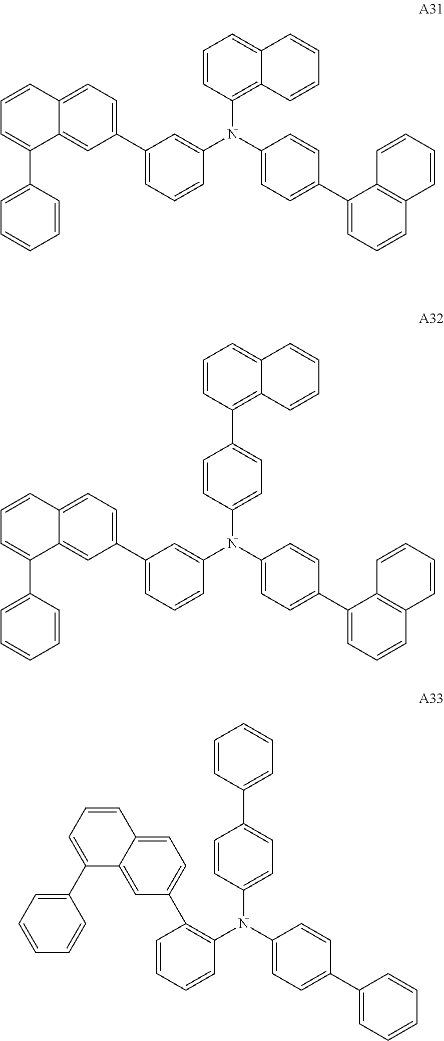 [SMILES: c1ccc(-c2cccc3ccc(-c4cccc(N(c5ccc(-c6cccc7ccccc67)cc5)c5cccc6ccccc56)c4)cc23)cc1 ; c1ccc(-c2cccc3ccc(-c4cccc(N(c5ccc(-c6cccc7ccccc67)cc5)c5ccc(-c6cccc7ccccc67)cc5)c4)cc23)cc1 ; c1ccc(-c2ccc(N(c3ccc(-c4ccccc4)cc3)c3ccccc3-c3ccc4cccc(-c5ccccc5)c4c3)cc2)cc1]

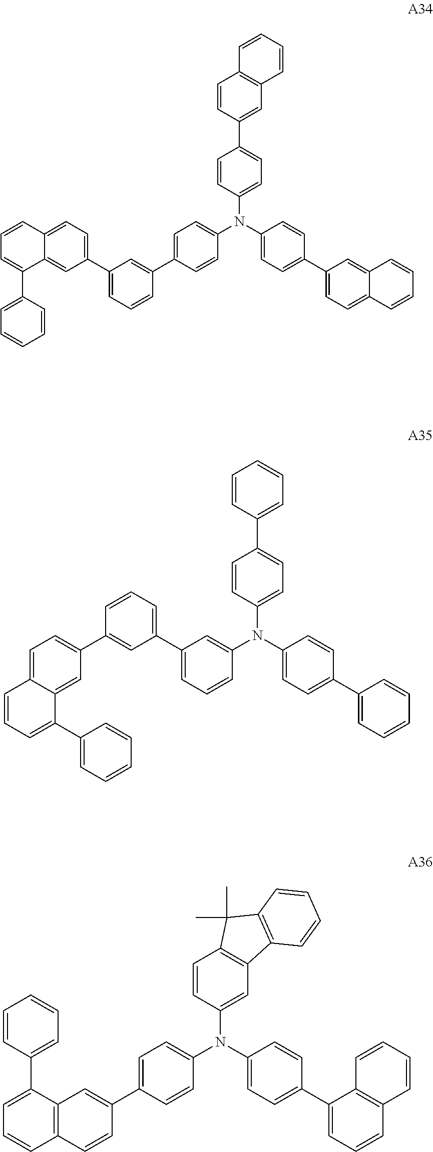 [SMILES: c1ccc(-c2cccc3ccc(-c4cccc(-c5ccc(N(c6ccc(-c7ccc8ccccc8c7)cc6)c6ccc(-c7ccc8ccccc8c7)cc6)cc5)c4)cc23)cc1 ; c1ccc(-c2ccc(N(c3ccc(-c4ccccc4)cc3)c3cccc(-c4cccc(-c5ccc6cccc(-c7ccccc7)c6c5)c4)c3)cc2)cc1 ; CC1(C)c2ccccc2-c2cc(N(c3ccc(-c4ccc5cccc(-c6ccccc6)c5c4)cc3)c3ccc(-c4cccc5ccccc45)cc3)ccc21]

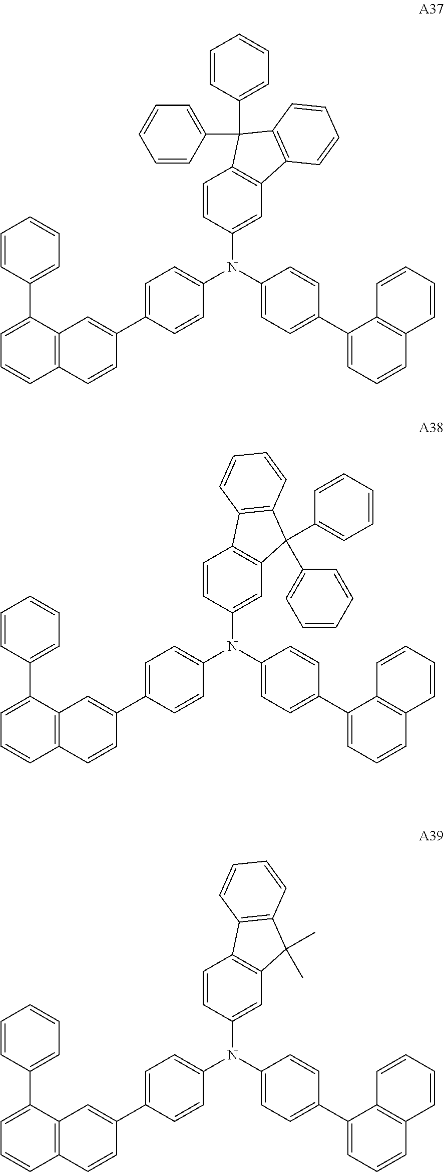 [SMILES: c1ccc(-c2cccc3ccc(-c4ccc(N(c5ccc(-c6cccc7ccccc67)cc5)c5ccc6c(c5)-c5ccccc5C6(c5ccccc5)c5ccccc5)cc4)cc23)cc1 ; c1ccc(-c2cccc3ccc(-c4ccc(N(c5ccc(-c6cccc7ccccc67)cc5)c5ccc6c(c5)C(c5ccccc5)(c5ccccc5)c5ccccc5-6)cc4)cc23)cc1 ; CC1(C)c2ccccc2-c2ccc(N(c3ccc(-c4ccc5cccc(-c6ccccc6)c5c4)cc3)c3ccc(-c4cccc5ccccc45)cc3)cc21]

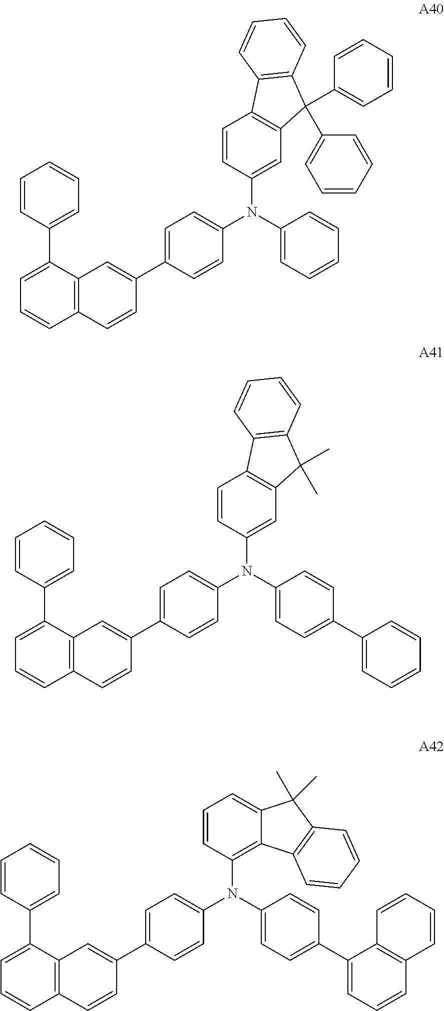 [SMILES: c1ccc(-c2cccc3ccc(-c4ccc(N(c5ccccc5)c5ccc6c(c5)C(c5ccccc5)(c5ccccc5)c5ccccc5-6)cc4)cc23)cc1 ; CC1(C)c2ccccc2-c2ccc(N(c3ccc(-c4ccccc4)cc3)c3ccc(-c4ccc5cccc(-c6ccccc6)c5c4)cc3)cc21 ; CC1(C)c2ccccc2-c2c(N(c3ccc(-c4ccc5cccc(-c6ccccc6)c5c4)cc3)c3ccc(-c4cccc5ccccc45)cc3)cccc21]

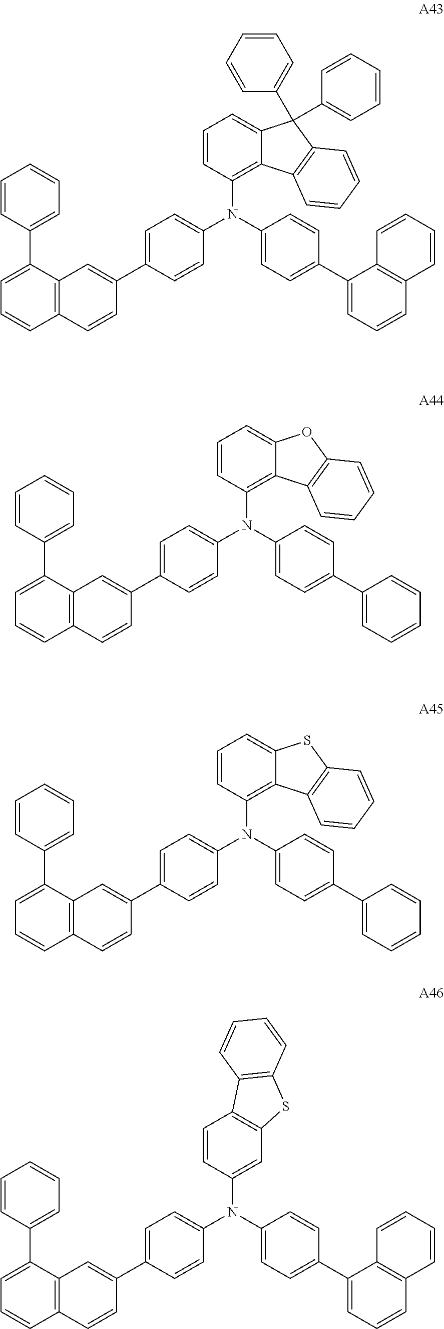 [SMILES: c1ccc(-c2cccc3ccc(-c4ccc(N(c5ccc(-c6cccc7ccccc67)cc5)c5cccc6c5-c5ccccc5C6(c5ccccc5)c5ccccc5)cc4)cc23)cc1 ; c1ccc(-c2ccc(N(c3ccc(-c4ccc5cccc(-c6ccccc6)c5c4)cc3)c3cccc4oc5ccccc5c34)cc2)cc1 ; c1ccc(-c2ccc(N(c3ccc(-c4ccc5cccc(-c6ccccc6)c5c4)cc3)c3cccc4sc5ccccc5c34)cc2)cc1 ; c1ccc(-c2cccc3ccc(-c4ccc(N(c5ccc(-c6cccc7ccccc67)cc5)c5ccc6c(c5)sc5ccccc56)cc4)cc23)cc1]

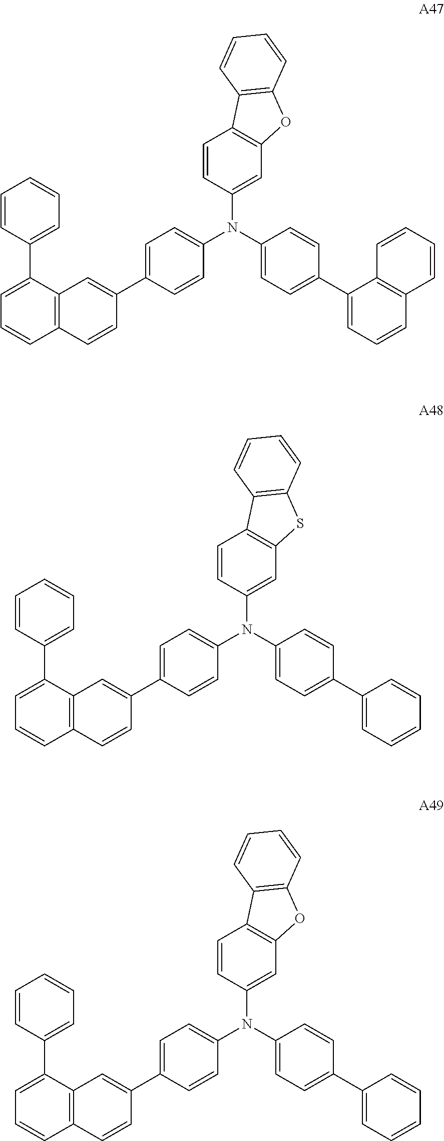 [SMILES: c1ccc(-c2cccc3ccc(-c4ccc(N(c5ccc(-c6cccc7ccccc67)cc5)c5ccc6c(c5)oc5ccccc56)cc4)cc23)cc1 ; c1ccc(-c2ccc(N(c3ccc(-c4ccc5cccc(-c6ccccc6)c5c4)cc3)c3ccc4c(c3)sc3ccccc34)cc2)cc1 ; c1ccc(-c2ccc(N(c3ccc(-c4ccc5cccc(-c6ccccc6)c5c4)cc3)c3ccc4c(c3)oc3ccccc34)cc2)cc1]

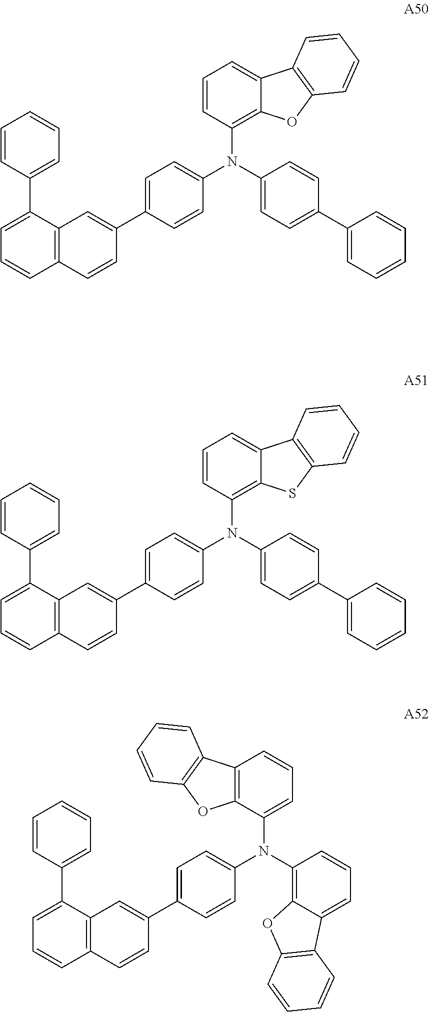 [SMILES: c1ccc(-c2ccc(N(c3ccc(-c4ccc5cccc(-c6ccccc6)c5c4)cc3)c3cccc4c3oc3ccccc34)cc2)cc1 ; c1ccc(-c2ccc(N(c3ccc(-c4ccc5cccc(-c6ccccc6)c5c4)cc3)c3cccc4c3sc3ccccc34)cc2)cc1 ; c1ccc(-c2cccc3ccc(-c4ccc(N(c5cccc6c5oc5ccccc56)c5cccc6c5oc5ccccc56)cc4)cc23)cc1]

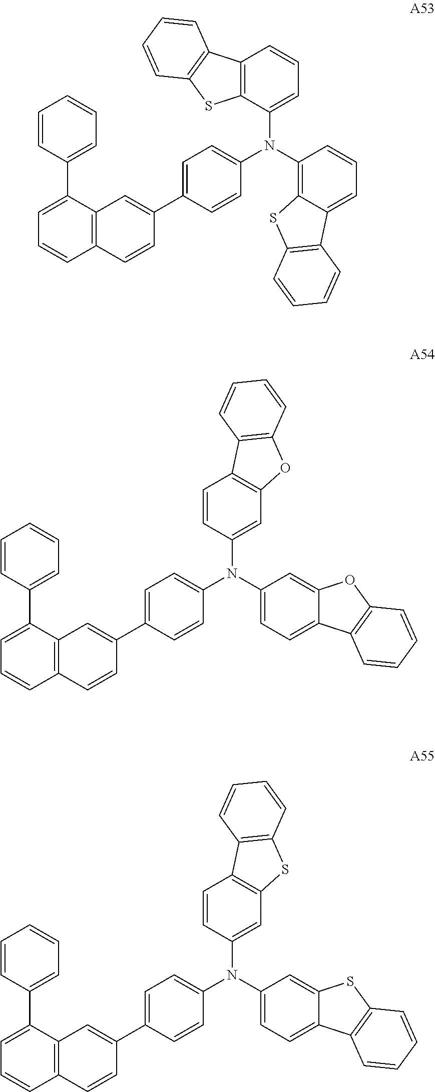 [SMILES: c1ccc(-c2cccc3ccc(-c4ccc(N(c5cccc6c5sc5ccccc56)c5cccc6c5sc5ccccc56)cc4)cc23)cc1 ; c1ccc(-c2cccc3ccc(-c4ccc(N(c5ccc6c(c5)oc5ccccc56)c5ccc6c(c5)oc5ccccc56)cc4)cc23)cc1 ; c1ccc(-c2cccc3ccc(-c4ccc(N(c5ccc6c(c5)sc5ccccc56)c5ccc6c(c5)sc5ccccc56)cc4)cc23)cc1]

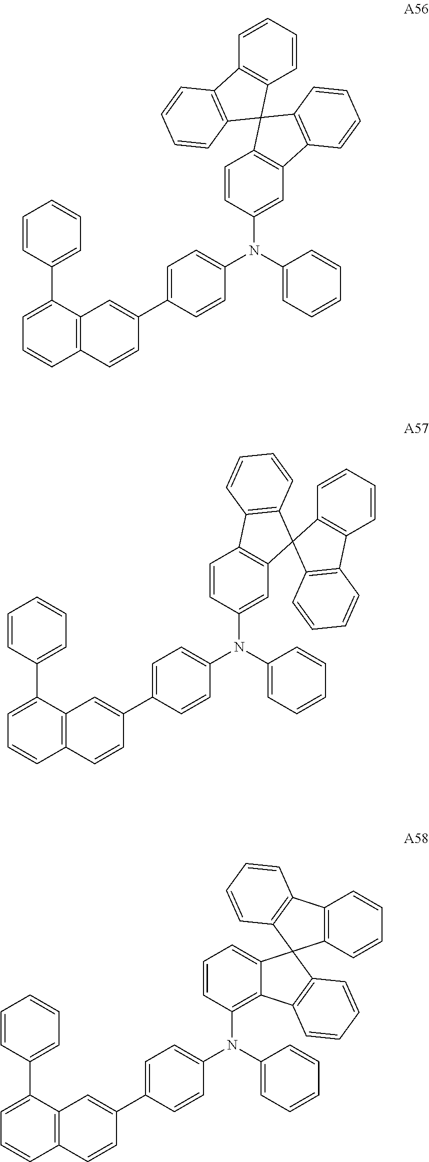 [SMILES: c1ccc(-c2cccc3ccc(-c4ccc(N(c5ccccc5)c5ccc6c(c5)-c5ccccc5C65c6ccccc6-c6ccccc65)cc4)cc23)cc1 ; c1ccc(-c2cccc3ccc(-c4ccc(N(c5ccccc5)c5ccc6c(c5)C5(c7ccccc7-c7ccccc75)c5ccccc5-6)cc4)cc23)cc1 ; c1ccc(-c2cccc3ccc(-c4ccc(N(c5ccccc5)c5cccc6c5-c5ccccc5C65c6ccccc6-c6ccccc65)cc4)cc23)cc1]

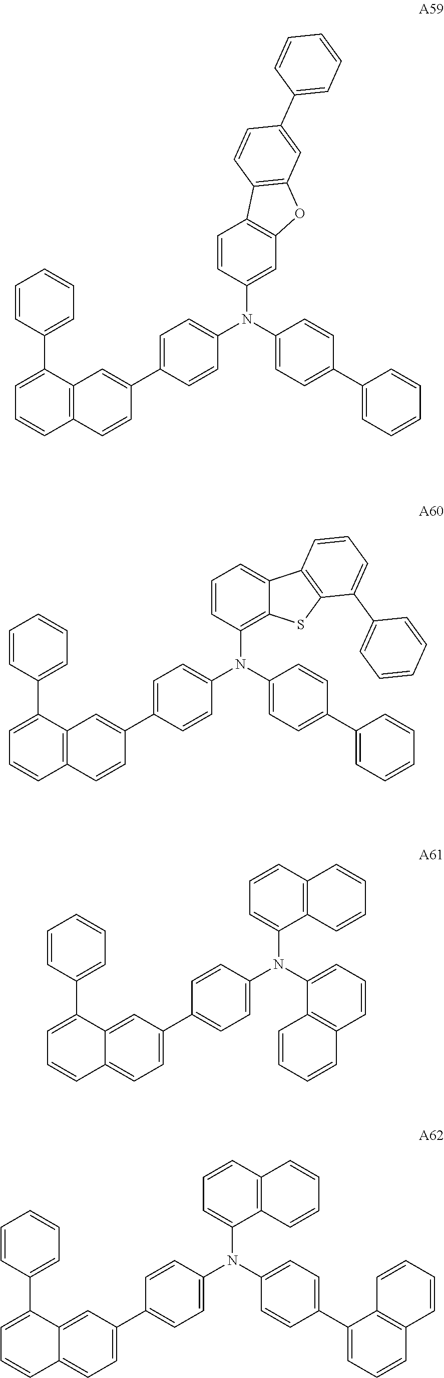 [SMILES: c1ccc(-c2ccc(N(c3ccc(-c4ccc5cccc(-c6ccccc6)c5c4)cc3)c3ccc4c(c3)oc3cc(-c5ccccc5)ccc34)cc2)cc1 ; c1ccc(-c2ccc(N(c3ccc(-c4ccc5cccc(-c6ccccc6)c5c4)cc3)c3cccc4c3sc3c(-c5ccccc5)cccc34)cc2)cc1 ; c1ccc(-c2cccc3ccc(-c4ccc(N(c5cccc6ccccc56)c5cccc6ccccc56)cc4)cc23)cc1 ; c1ccc(-c2cccc3ccc(-c4ccc(N(c5ccc(-c6cccc7ccccc67)cc5)c5cccc6ccccc56)cc4)cc23)cc1]

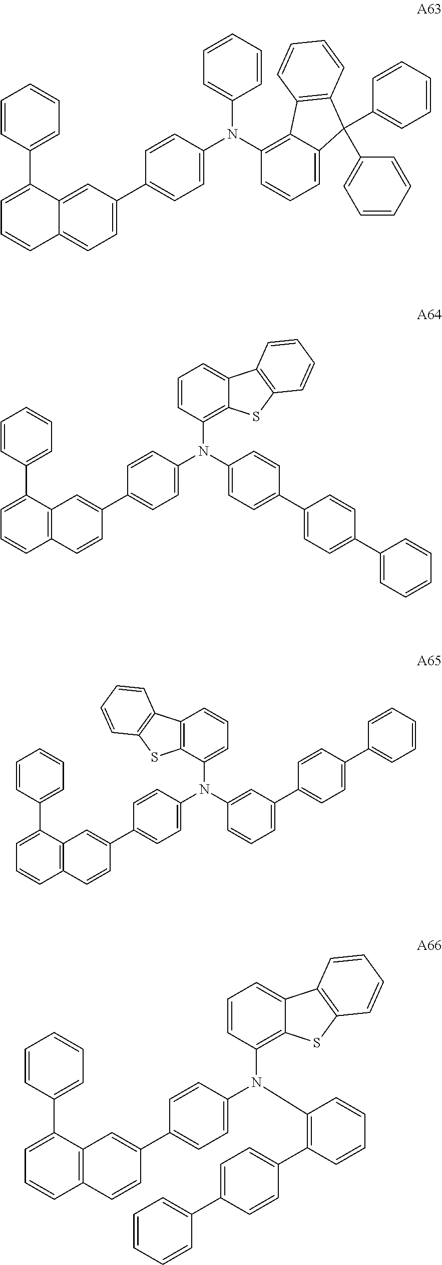 [SMILES: c1ccc(-c2cccc3ccc(-c4ccc(N(c5ccccc5)c5cccc6c5-c5ccccc5C6(c5ccccc5)c5ccccc5)cc4)cc23)cc1 ; c1ccc(-c2ccc(-c3ccc(N(c4ccc(-c5ccc6cccc(-c7ccccc7)c6c5)cc4)c4cccc5c4sc4ccccc45)cc3)cc2)cc1 ; c1ccc(-c2ccc(-c3cccc(N(c4ccc(-c5ccc6cccc(-c7ccccc7)c6c5)cc4)c4cccc5c4sc4ccccc45)c3)cc2)cc1 ; c1ccc(-c2ccc(-c3ccccc3N(c3ccc(-c4ccc5cccc(-c6ccccc6)c5c4)cc3)c3cccc4c3sc3ccccc34)cc2)cc1]

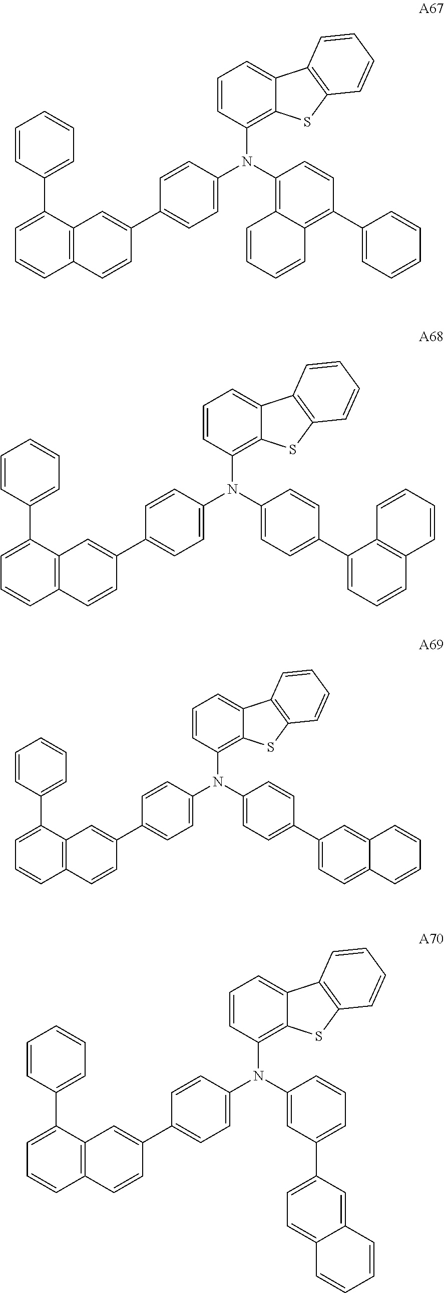 [SMILES: c1ccc(-c2cccc3ccc(-c4ccc(N(c5ccc(-c6ccccc6)c6ccccc56)c5cccc6c5sc5ccccc56)cc4)cc23)cc1 ; c1ccc(-c2cccc3ccc(-c4ccc(N(c5ccc(-c6cccc7ccccc67)cc5)c5cccc6c5sc5ccccc56)cc4)cc23)cc1 ; c1ccc(-c2cccc3ccc(-c4ccc(N(c5ccc(-c6ccc7ccccc7c6)cc5)c5cccc6c5sc5ccccc56)cc4)cc23)cc1 ; c1ccc(-c2cccc3ccc(-c4ccc(N(c5cccc(-c6ccc7ccccc7c6)c5)c5cccc6c5sc5ccccc56)cc4)cc23)cc1]

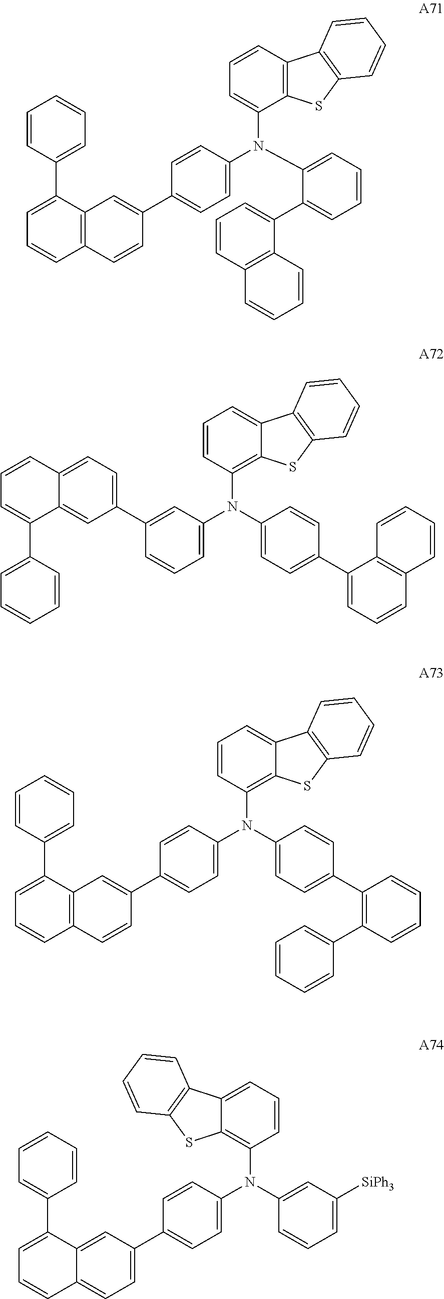

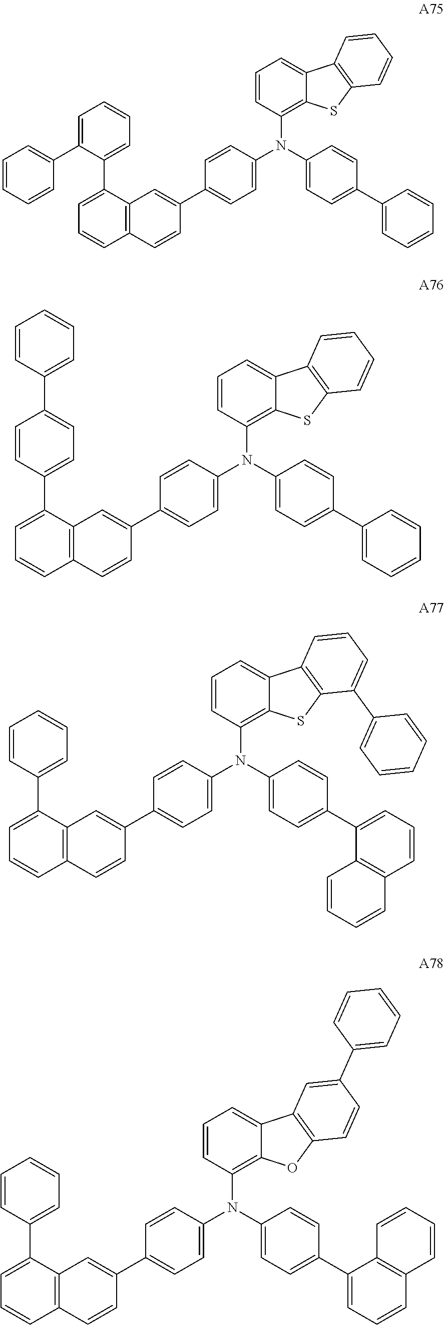 [SMILES: c1ccc(-c2ccc(N(c3ccc(-c4ccc5cccc(-c6ccccc6-c6ccccc6)c5c4)cc3)c3cccc4c3sc3ccccc34)cc2)cc1 ; c1ccc(-c2ccc(-c3cccc4ccc(-c5ccc(N(c6ccc(-c7ccccc7)cc6)c6cccc7c6sc6ccccc67)cc5)cc34)cc2)cc1 ; c1ccc(-c2cccc3ccc(-c4ccc(N(c5ccc(-c6cccc7ccccc67)cc5)c5cccc6c5sc5c(-c7ccccc7)cccc56)cc4)cc23)cc1 ; c1ccc(-c2ccc3oc4c(N(c5ccc(-c6ccc7cccc(-c8ccccc8)c7c6)cc5)c5ccc(-c6cccc7ccccc67)cc5)cccc4c3c2)cc1]

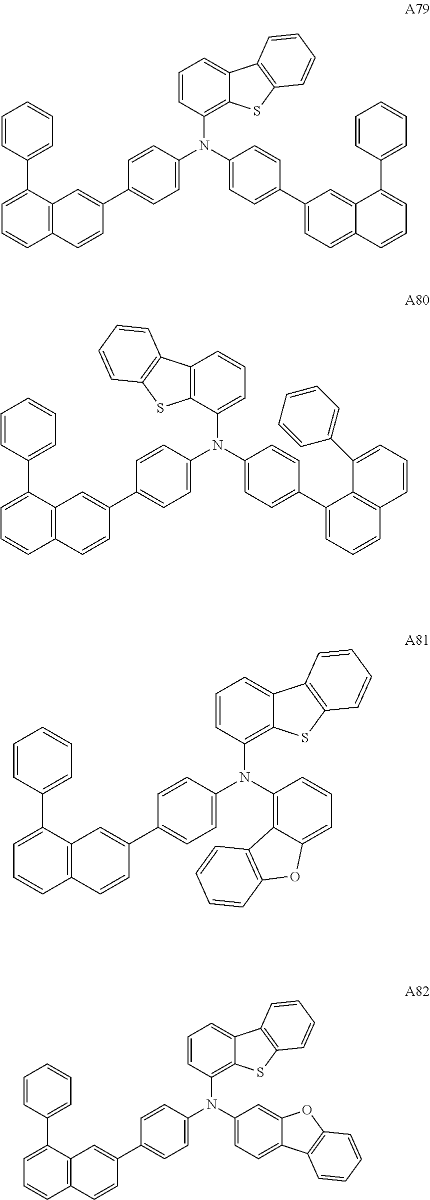 [SMILES: c1ccc(-c2cccc3ccc(-c4ccc(N(c5ccc(-c6ccc7cccc(-c8ccccc8)c7c6)cc5)c5cccc6c5sc5ccccc56)cc4)cc23)cc1 ; c1ccc(-c2cccc3ccc(-c4ccc(N(c5ccc(-c6cccc7cccc(-c8ccccc8)c67)cc5)c5cccc6c5sc5ccccc56)cc4)cc23)cc1 ; c1ccc(-c2cccc3ccc(-c4ccc(N(c5cccc6c5sc5ccccc56)c5cccc6oc7ccccc7c56)cc4)cc23)cc1 ; c1ccc(-c2cccc3ccc(-c4ccc(N(c5ccc6c(c5)oc5ccccc56)c5cccc6c5sc5ccccc56)cc4)cc23)cc1]

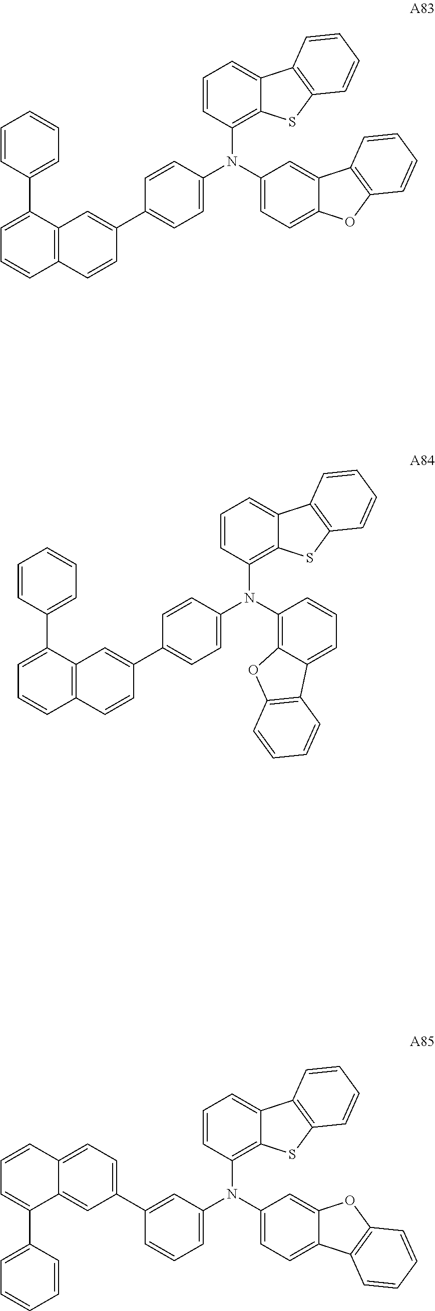 [SMILES: c1ccc(-c2cccc3ccc(-c4ccc(N(c5ccc6oc7ccccc7c6c5)c5cccc6c5sc5ccccc56)cc4)cc23)cc1 ; c1ccc(-c2cccc3ccc(-c4ccc(N(c5cccc6c5oc5ccccc56)c5cccc6c5sc5ccccc56)cc4)cc23)cc1 ; c1ccc(-c2cccc3ccc(-c4cccc(N(c5ccc6c(c5)oc5ccccc56)c5cccc6c5sc5ccccc56)c4)cc23)cc1]

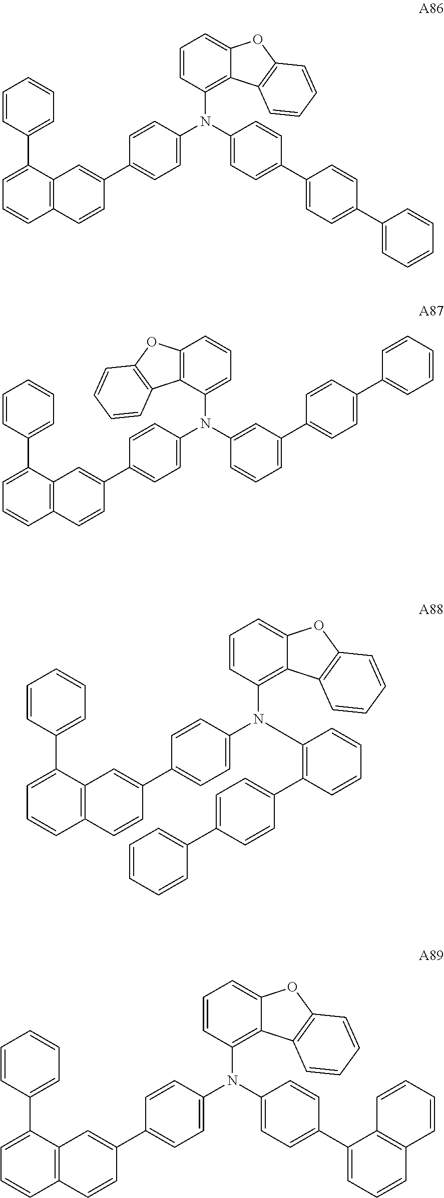 [SMILES: c1ccc(-c2ccc(-c3ccc(N(c4ccc(-c5ccc6cccc(-c7ccccc7)c6c5)cc4)c4cccc5oc6ccccc6c45)cc3)cc2)cc1 ; c1ccc(-c2ccc(-c3cccc(N(c4ccc(-c5ccc6cccc(-c7ccccc7)c6c5)cc4)c4cccc5oc6ccccc6c45)c3)cc2)cc1 ; c1ccc(-c2ccc(-c3ccccc3N(c3ccc(-c4ccc5cccc(-c6ccccc6)c5c4)cc3)c3cccc4oc5ccccc5c34)cc2)cc1 ; c1ccc(-c2cccc3ccc(-c4ccc(N(c5ccc(-c6cccc7ccccc67)cc5)c5cccc6oc7ccccc7c56)cc4)cc23)cc1]

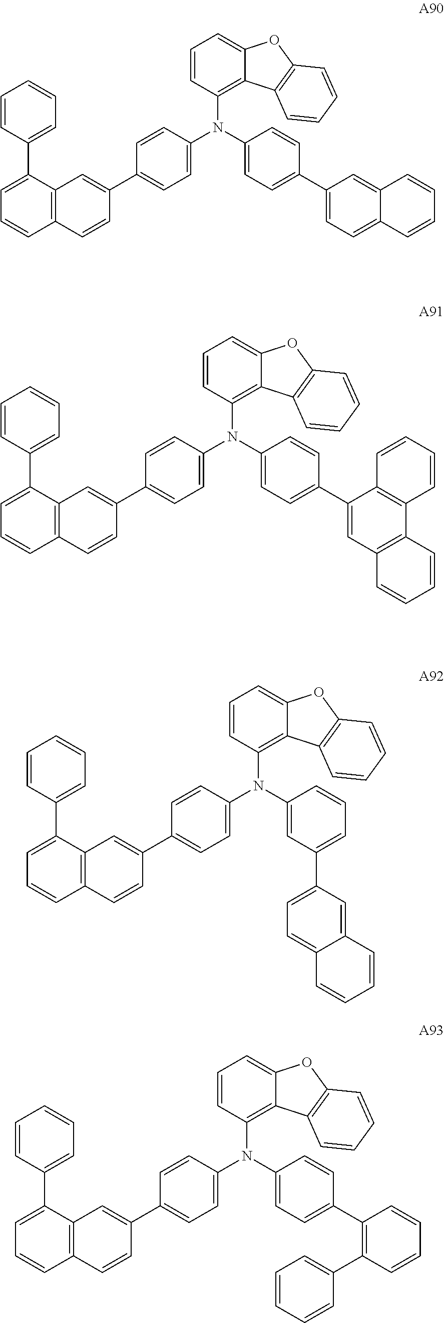 [SMILES: c1ccc(-c2cccc3ccc(-c4ccc(N(c5ccc(-c6ccc7ccccc7c6)cc5)c5cccc6oc7ccccc7c56)cc4)cc23)cc1 ; c1ccc(-c2cccc3ccc(-c4ccc(N(c5ccc(-c6cc7ccccc7c7ccccc67)cc5)c5cccc6oc7ccccc7c56)cc4)cc23)cc1 ; c1ccc(-c2cccc3ccc(-c4ccc(N(c5cccc(-c6ccc7ccccc7c6)c5)c5cccc6oc7ccccc7c56)cc4)cc23)cc1 ; c1ccc(-c2ccccc2-c2ccc(N(c3ccc(-c4ccc5cccc(-c6ccccc6)c5c4)cc3)c3cccc4oc5ccccc5c34)cc2)cc1]

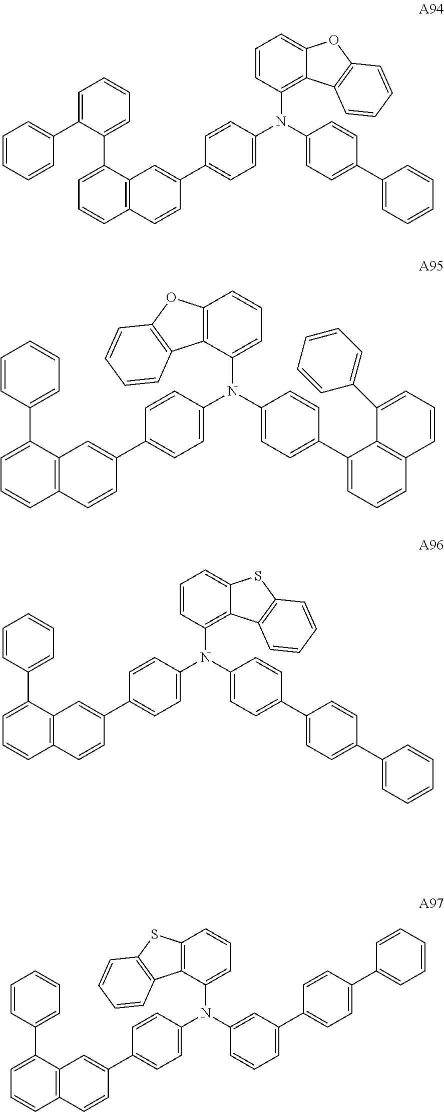 [SMILES: c1ccc(-c2ccc(N(c3ccc(-c4ccc5cccc(-c6ccccc6-c6ccccc6)c5c4)cc3)c3cccc4oc5ccccc5c34)cc2)cc1 ; c1ccc(-c2cccc3ccc(-c4ccc(N(c5ccc(-c6cccc7cccc(-c8ccccc8)c67)cc5)c5cccc6oc7ccccc7c56)cc4)cc23)cc1 ; c1ccc(-c2ccc(-c3ccc(N(c4ccc(-c5ccc6cccc(-c7ccccc7)c6c5)cc4)c4cccc5sc6ccccc6c45)cc3)cc2)cc1 ; c1ccc(-c2ccc(-c3cccc(N(c4ccc(-c5ccc6cccc(-c7ccccc7)c6c5)cc4)c4cccc5sc6ccccc6c45)c3)cc2)cc1]

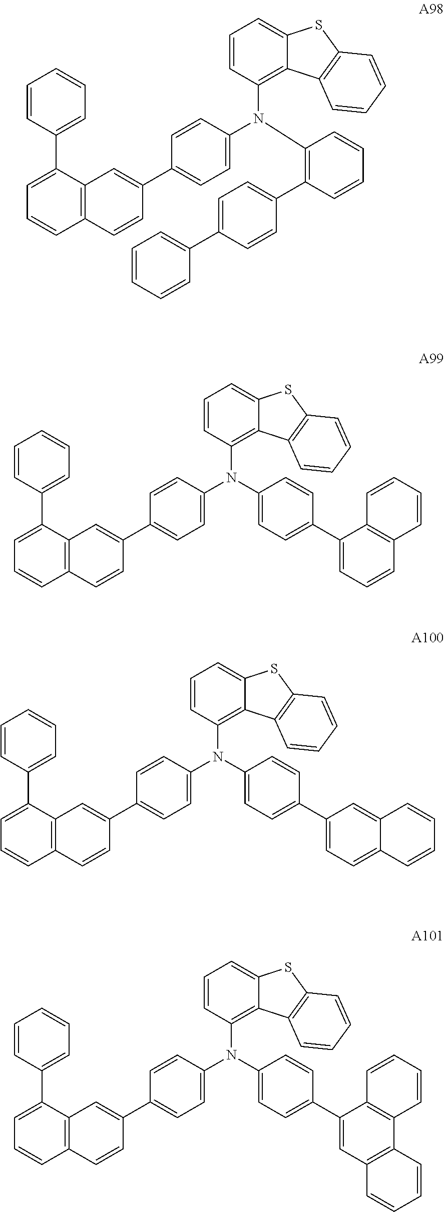 [SMILES: c1ccc(-c2ccc(-c3ccccc3N(c3ccc(-c4ccc5cccc(-c6ccccc6)c5c4)cc3)c3cccc4sc5ccccc5c34)cc2)cc1 ; c1ccc(-c2cccc3ccc(-c4ccc(N(c5ccc(-c6cccc7ccccc67)cc5)c5cccc6sc7ccccc7c56)cc4)cc23)cc1 ; c1ccc(-c2cccc3ccc(-c4ccc(N(c5ccc(-c6ccc7ccccc7c6)cc5)c5cccc6sc7ccccc7c56)cc4)cc23)cc1 ; c1ccc(-c2cccc3ccc(-c4ccc(N(c5ccc(-c6cc7ccccc7c7ccccc67)cc5)c5cccc6sc7ccccc7c56)cc4)cc23)cc1]

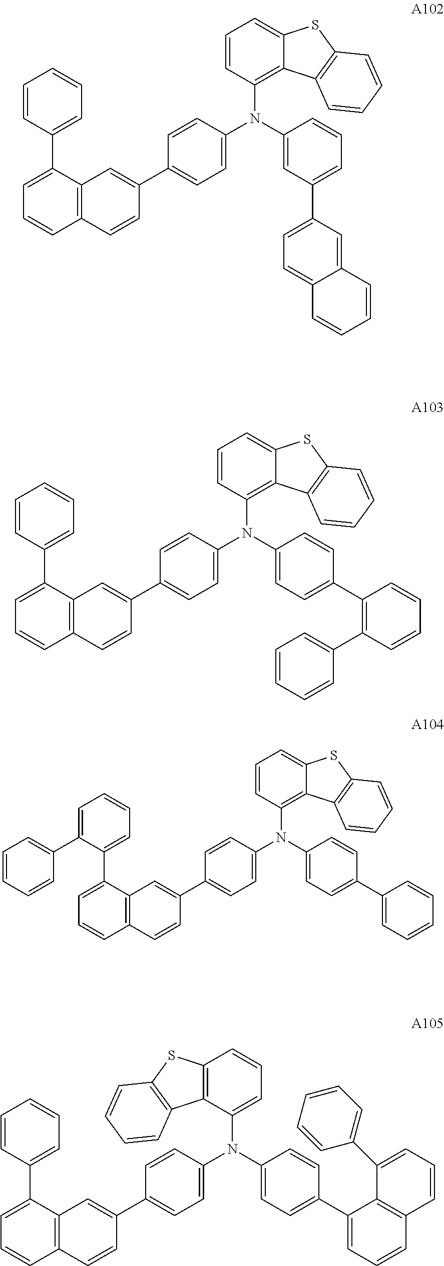 [SMILES: c1ccc(-c2cccc3ccc(-c4ccc(N(c5cccc(-c6ccc7ccccc7c6)c5)c5cccc6sc7ccccc7c56)cc4)cc23)cc1 ; c1ccc(-c2ccccc2-c2ccc(N(c3ccc(-c4ccc5cccc(-c6ccccc6)c5c4)cc3)c3cccc4sc5ccccc5c34)cc2)cc1 ; c1ccc(-c2ccc(N(c3ccc(-c4ccc5cccc(-c6ccccc6-c6ccccc6)c5c4)cc3)c3cccc4sc5ccccc5c34)cc2)cc1 ; c1ccc(-c2cccc3ccc(-c4ccc(N(c5ccc(-c6cccc7cccc(-c8ccccc8)c67)cc5)c5cccc6sc7ccccc7c56)cc4)cc23)cc1]

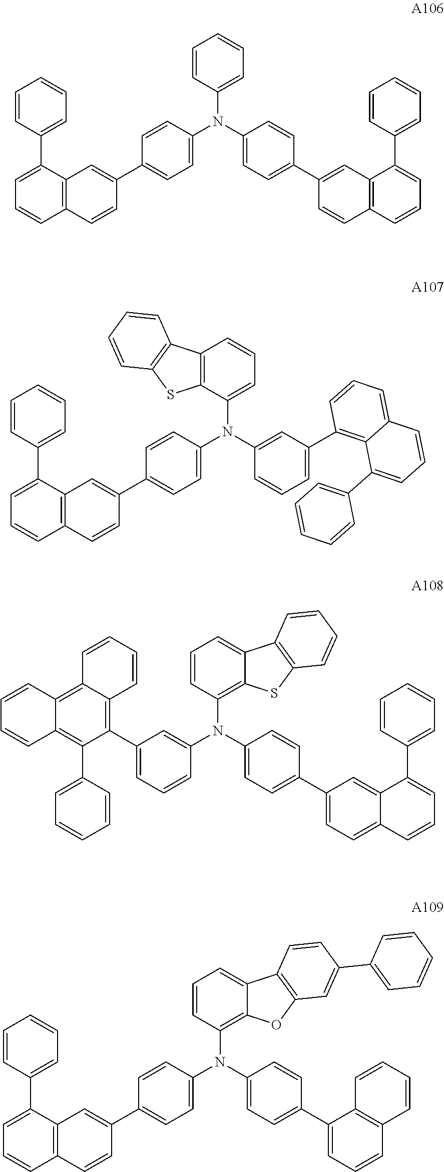 [SMILES: c1ccc(-c2cccc3ccc(-c4ccc(N(c5ccccc5)c5ccc(-c6ccc7cccc(-c8ccccc8)c7c6)cc5)cc4)cc23)cc1 ; c1ccc(-c2cccc3ccc(-c4ccc(N(c5cccc(-c6cccc7cccc(-c8ccccc8)c67)c5)c5cccc6c5sc5ccccc56)cc4)cc23)cc1 ; c1ccc(-c2cccc3ccc(-c4ccc(N(c5cccc(-c6c(-c7ccccc7)c7ccccc7c7ccccc67)c5)c5cccc6c5sc5ccccc56)cc4)cc23)cc1 ; c1ccc(-c2ccc3c(c2)oc2c(N(c4ccc(-c5ccc6cccc(-c7ccccc7)c6c5)cc4)c4ccc(-c5cccc6ccccc56)cc4)cccc23)cc1]

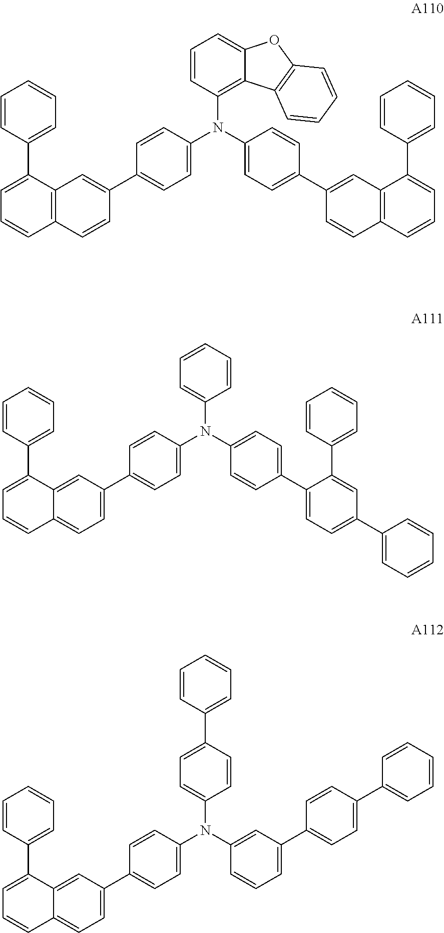 [SMILES: c1ccc(-c2cccc3ccc(-c4ccc(N(c5ccc(-c6ccc7cccc(-c8ccccc8)c7c6)cc5)c5cccc6oc7ccccc7c56)cc4)cc23)cc1 ; c1ccc(-c2ccc(-c3ccc(N(c4ccccc4)c4ccc(-c5ccc6cccc(-c7ccccc7)c6c5)cc4)cc3)c(-c3ccccc3)c2)cc1 ; c1ccc(-c2ccc(-c3cccc(N(c4ccc(-c5ccccc5)cc4)c4ccc(-c5ccc6cccc(-c7ccccc7)c6c5)cc4)c3)cc2)cc1]

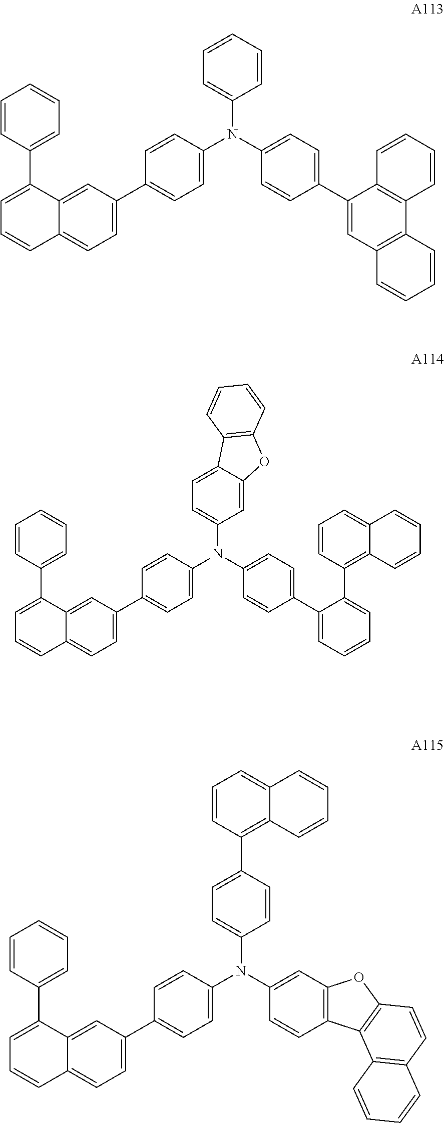 [SMILES: c1ccc(-c2cccc3ccc(-c4ccc(N(c5ccccc5)c5ccc(-c6cc7ccccc7c7ccccc67)cc5)cc4)cc23)cc1 ; c1ccc(-c2cccc3ccc(-c4ccc(N(c5ccc(-c6ccccc6-c6cccc7ccccc67)cc5)c5ccc6c(c5)oc5ccccc56)cc4)cc23)cc1 ; c1ccc(-c2cccc3ccc(-c4ccc(N(c5ccc(-c6cccc7ccccc67)cc5)c5ccc6c(c5)oc5ccc7ccccc7c56)cc4)cc23)cc1]

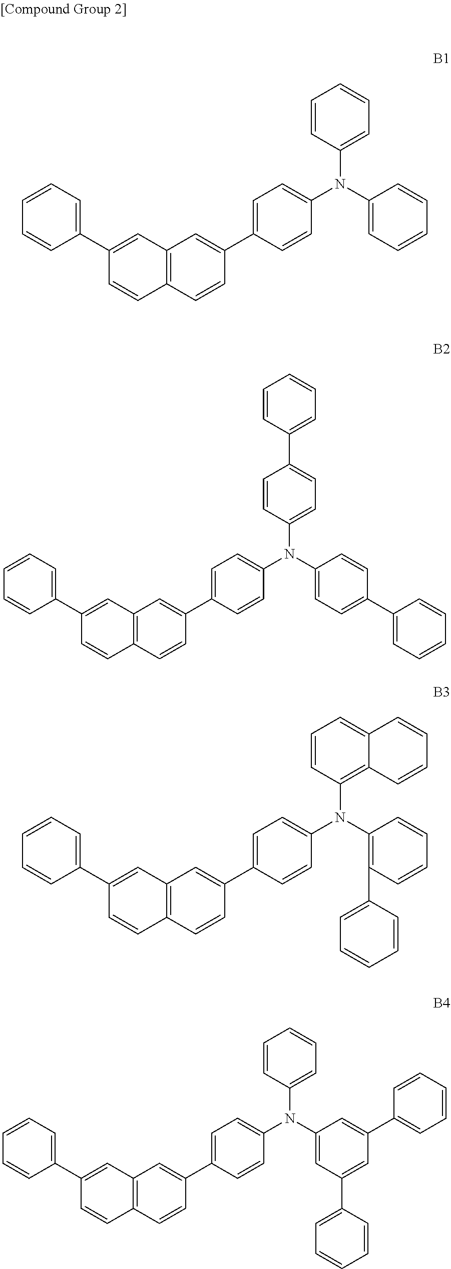 [SMILES: c1ccc(-c2ccc3ccc(-c4ccc(N(c5ccccc5)c5ccccc5)cc4)cc3c2)cc1 ; c1ccc(-c2ccc(N(c3ccc(-c4ccccc4)cc3)c3ccc(-c4ccc5ccc(-c6ccccc6)cc5c4)cc3)cc2)cc1 ; c1ccc(-c2ccc3ccc(-c4ccc(N(c5ccccc5-c5ccccc5)c5cccc6ccccc56)cc4)cc3c2)cc1 ; c1ccc(-c2cc(-c3ccccc3)cc(N(c3ccccc3)c3ccc(-c4ccc5ccc(-c6ccccc6)cc5c4)cc3)c2)cc1]

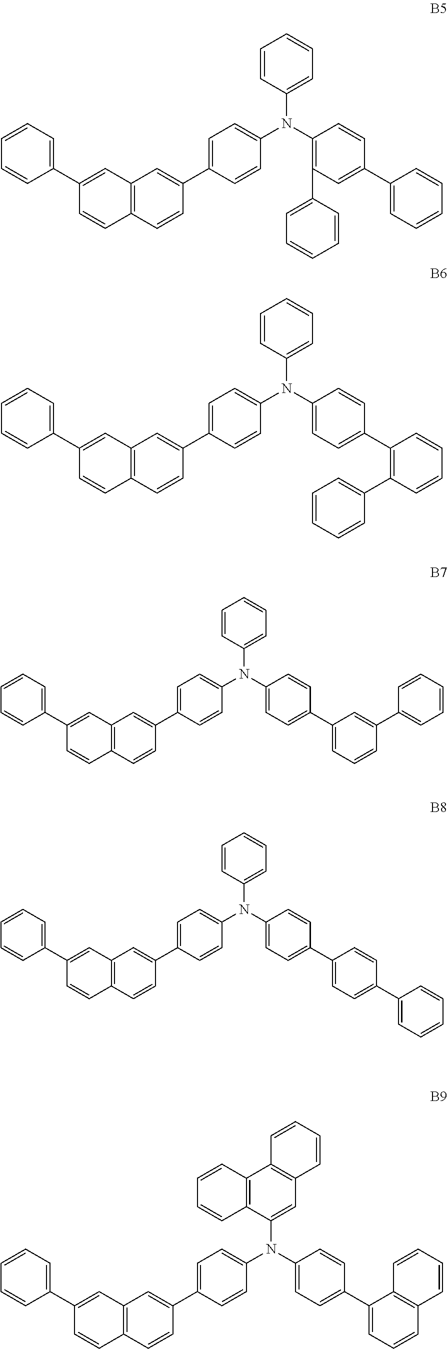 [SMILES: c1ccc(-c2ccc(N(c3ccccc3)c3ccc(-c4ccc5ccc(-c6ccccc6)cc5c4)cc3)c(-c3ccccc3)c2)cc1 ; c1ccc(-c2ccc3ccc(-c4ccc(N(c5ccccc5)c5ccc(-c6ccccc6-c6ccccc6)cc5)cc4)cc3c2)cc1 ; c1ccc(-c2cccc(-c3ccc(N(c4ccccc4)c4ccc(-c5ccc6ccc(-c7ccccc7)cc6c5)cc4)cc3)c2)cc1 ; c1ccc(-c2ccc(-c3ccc(N(c4ccccc4)c4ccc(-c5ccc6ccc(-c7ccccc7)cc6c5)cc4)cc3)cc2)cc1 ; c1ccc(-c2ccc3ccc(-c4ccc(N(c5ccc(-c6cccc7ccccc67)cc5)c5cc6ccccc6c6ccccc56)cc4)cc3c2)cc1]

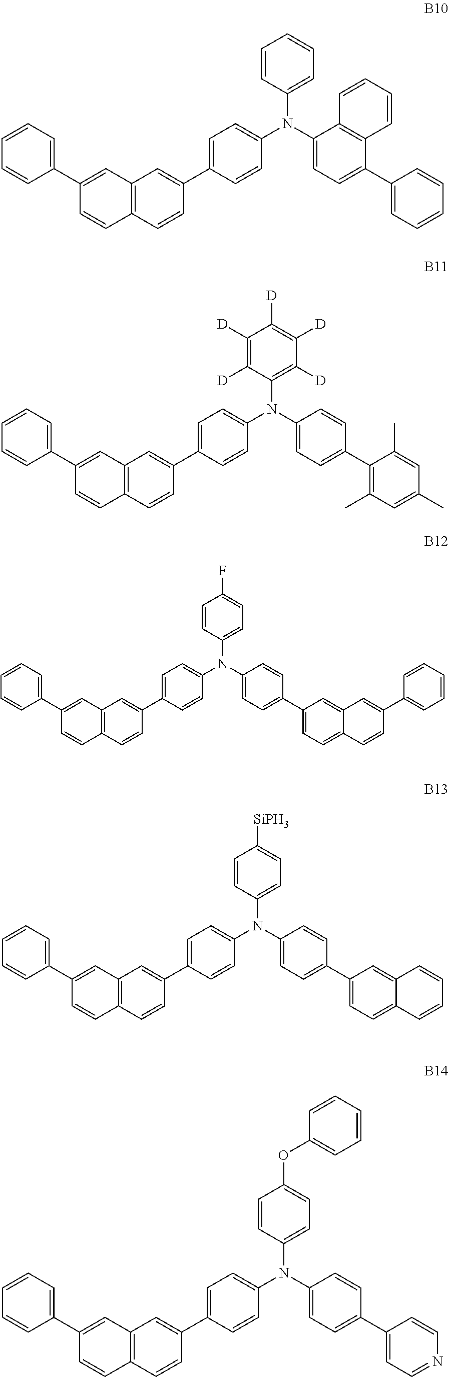

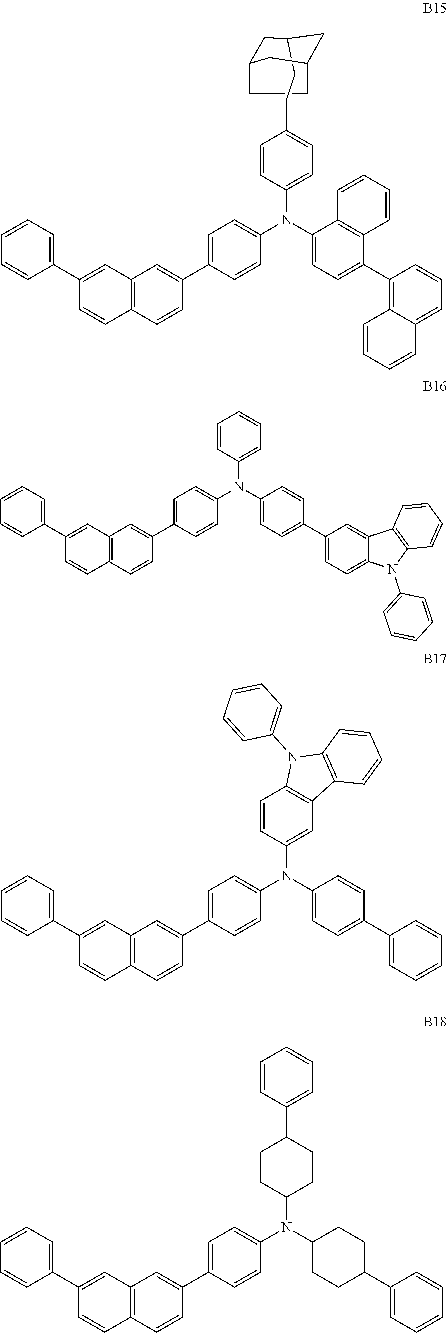 [SMILES: c1ccc(-c2ccc3ccc(-c4ccc(N(c5ccc(CCC6CC7CCC(C6)C7)cc5)c5ccc(-c6cccc7ccccc67)c6ccccc56)cc4)cc3c2)cc1 ; c1ccc(-c2ccc3ccc(-c4ccc(N(c5ccccc5)c5ccc(-c6ccc7c(c6)c6ccccc6n7-c6ccccc6)cc5)cc4)cc3c2)cc1 ; c1ccc(-c2ccc(N(c3ccc(-c4ccc5ccc(-c6ccccc6)cc5c4)cc3)c3ccc4c(c3)c3ccccc3n4-c3ccccc3)cc2)cc1 ; c1ccc(-c2ccc3ccc(-c4ccc(N(C5CCC(c6ccccc6)CC5)C5CCC(c6ccccc6)CC5)cc4)cc3c2)cc1]

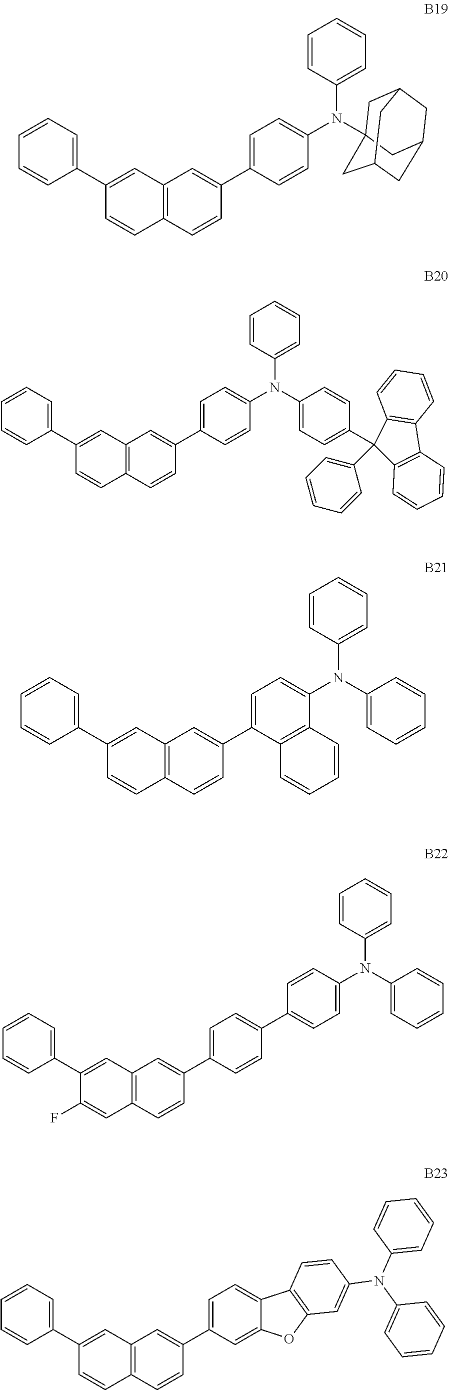 [SMILES: c1ccc(-c2ccc3ccc(-c4ccc(N(c5ccccc5)C56CC7CC(CC(C7)C5)C6)cc4)cc3c2)cc1 ; c1ccc(-c2ccc3ccc(-c4ccc(N(c5ccccc5)c5ccc(C6(c7ccccc7)c7ccccc7-c7ccccc76)cc5)cc4)cc3c2)cc1 ; c1ccc(-c2ccc3ccc(-c4ccc(N(c5ccccc5)c5ccccc5)c5ccccc45)cc3c2)cc1 ; Fc1cc2ccc(-c3ccc(-c4ccc(N(c5ccccc5)c5ccccc5)cc4)cc3)cc2cc1-c1ccccc1 ; c1ccc(-c2ccc3ccc(-c4ccc5c(c4)oc4cc(N(c6ccccc6)c6ccccc6)ccc45)cc3c2)cc1]

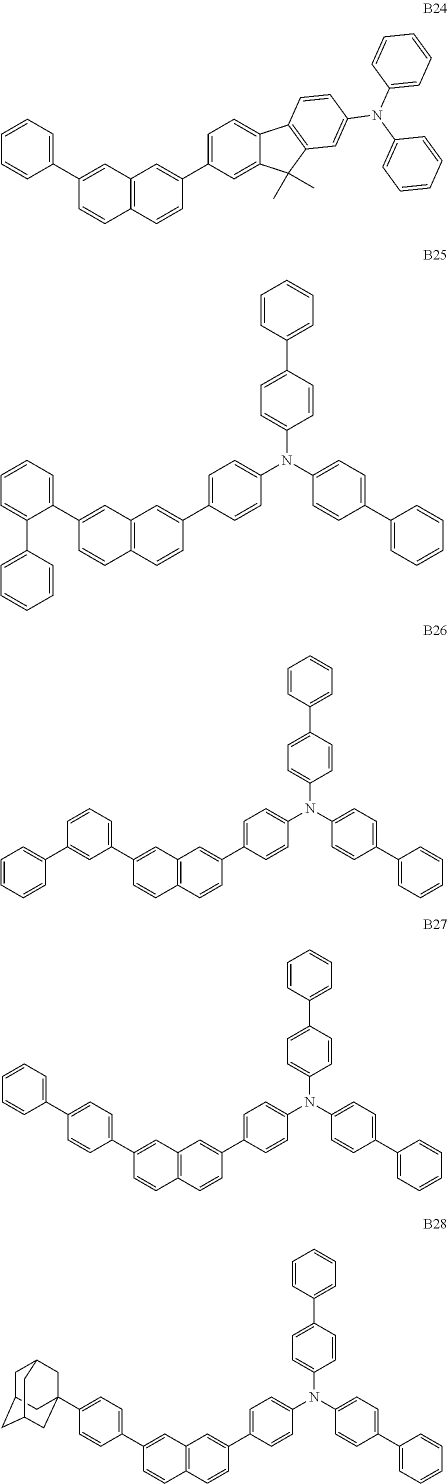 [SMILES: CC1(C)c2cc(-c3ccc4ccc(-c5ccccc5)cc4c3)ccc2-c2ccc(N(c3ccccc3)c3ccccc3)cc21 ; c1ccc(-c2ccc(N(c3ccc(-c4ccccc4)cc3)c3ccc(-c4ccc5ccc(-c6ccccc6-c6ccccc6)cc5c4)cc3)cc2)cc1 ; c1ccc(-c2ccc(N(c3ccc(-c4ccccc4)cc3)c3ccc(-c4ccc5ccc(-c6cccc(-c7ccccc7)c6)cc5c4)cc3)cc2)cc1 ; c1ccc(-c2ccc(-c3ccc4ccc(-c5ccc(N(c6ccc(-c7ccccc7)cc6)c6ccc(-c7ccccc7)cc6)cc5)cc4c3)cc2)cc1 ; c1ccc(-c2ccc(N(c3ccc(-c4ccccc4)cc3)c3ccc(-c4ccc5ccc(-c6ccc(C78CC9CC(CC(C9)C7)C8)cc6)cc5c4)cc3)cc2)cc1]

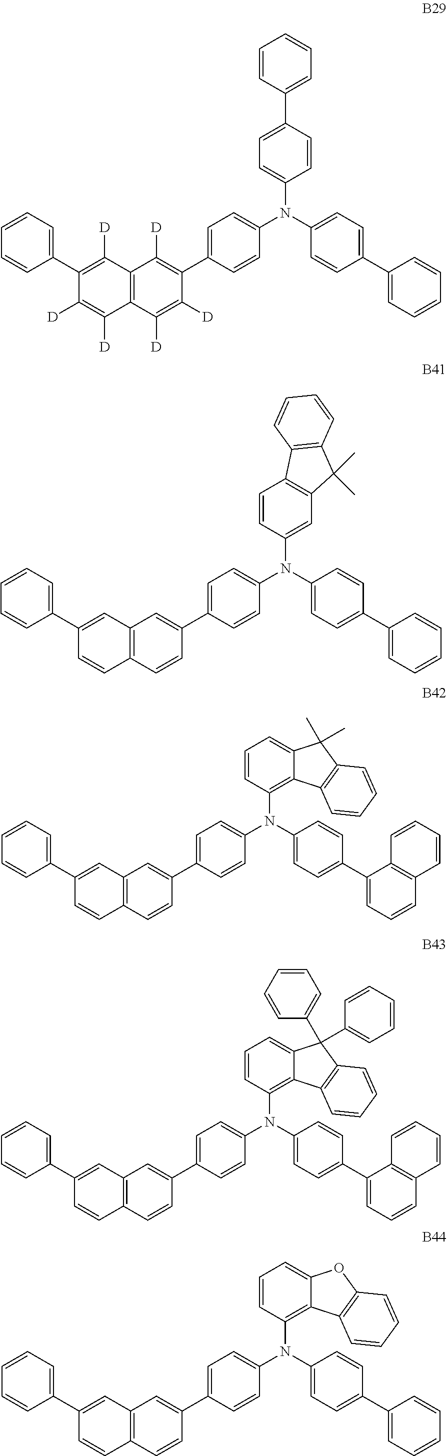 [SMILES: [2H]c1c(-c2ccccc2)c([2H])c2c([2H])c(-c3ccc(N(c4ccc(-c5ccccc5)cc4)c4ccc(-c5ccccc5)cc4)cc3)c([2H])c([2H])c2c1[2H] ; CC1(C)c2ccccc2-c2ccc(N(c3ccc(-c4ccccc4)cc3)c3ccc(-c4ccc5ccc(-c6ccccc6)cc5c4)cc3)cc21 ; CC1(C)c2ccccc2-c2c(N(c3ccc(-c4ccc5ccc(-c6ccccc6)cc5c4)cc3)c3ccc(-c4cccc5ccccc45)cc3)cccc21 ; c1ccc(-c2ccc3ccc(-c4ccc(N(c5ccc(-c6cccc7ccccc67)cc5)c5cccc6c5-c5ccccc5C6(c5ccccc5)c5ccccc5)cc4)cc3c2)cc1 ; c1ccc(-c2ccc(N(c3ccc(-c4ccc5ccc(-c6ccccc6)cc5c4)cc3)c3cccc4oc5ccccc5c34)cc2)cc1]

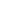

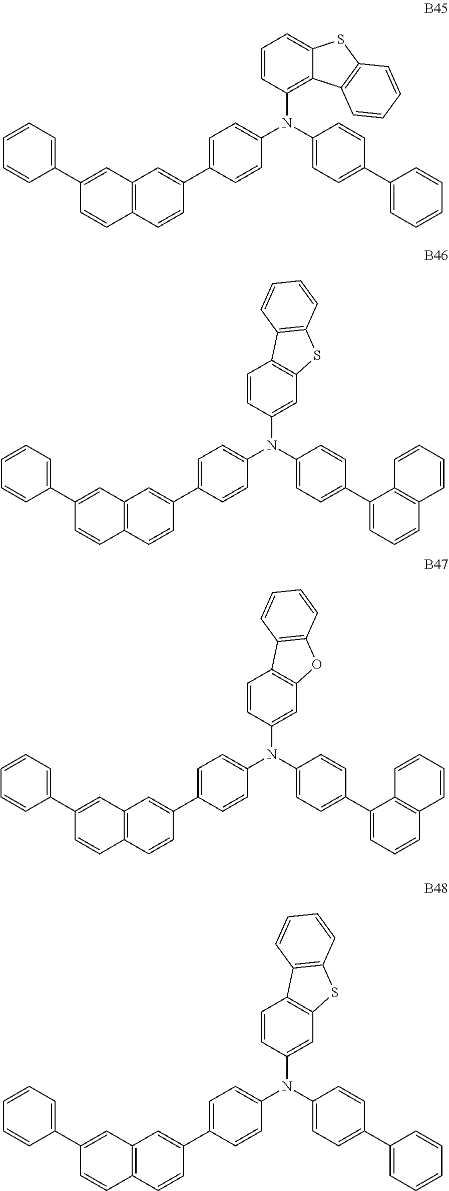 [SMILES: c1ccc(-c2ccc(N(c3ccc(-c4ccc5ccc(-c6ccccc6)cc5c4)cc3)c3cccc4sc5ccccc5c34)cc2)cc1 ; c1ccc(-c2ccc3ccc(-c4ccc(N(c5ccc(-c6cccc7ccccc67)cc5)c5ccc6c(c5)sc5ccccc56)cc4)cc3c2)cc1 ; c1ccc(-c2ccc3ccc(-c4ccc(N(c5ccc(-c6cccc7ccccc67)cc5)c5ccc6c(c5)oc5ccccc56)cc4)cc3c2)cc1 ; c1ccc(-c2ccc(N(c3ccc(-c4ccc5ccc(-c6ccccc6)cc5c4)cc3)c3ccc4c(c3)sc3ccccc34)cc2)cc1]

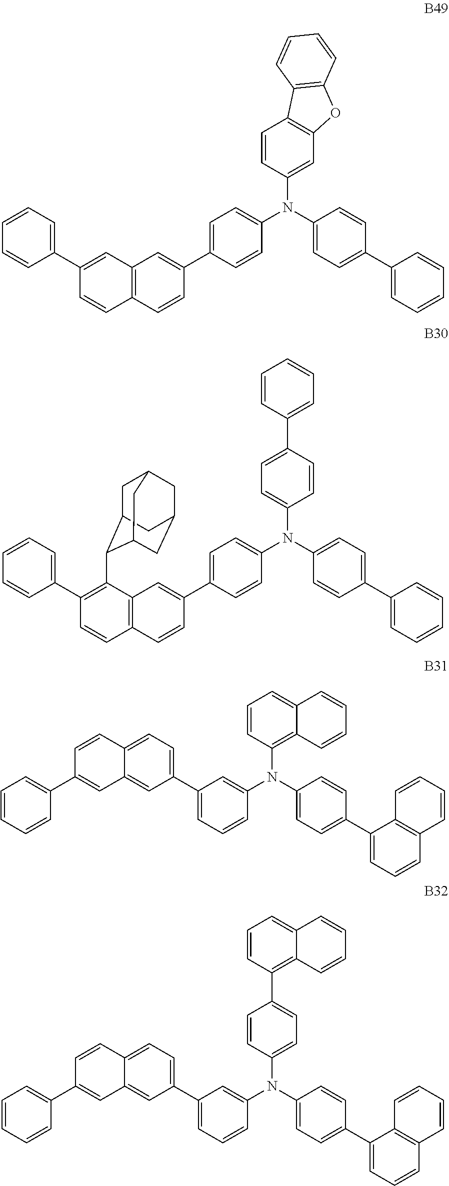 [SMILES: c1ccc(-c2ccc(N(c3ccc(-c4ccc5ccc(-c6ccccc6)cc5c4)cc3)c3ccc4c(c3)oc3ccccc34)cc2)cc1 ; c1ccc(-c2ccc(N(c3ccc(-c4ccccc4)cc3)c3ccc(-c4ccc5ccc(-c6ccccc6)c(C6C7CC8CC(C7)CC6C8)c5c4)cc3)cc2)cc1 ; c1ccc(-c2ccc3ccc(-c4cccc(N(c5ccc(-c6cccc7ccccc67)cc5)c5cccc6ccccc56)c4)cc3c2)cc1 ; c1ccc(-c2ccc3ccc(-c4cccc(N(c5ccc(-c6cccc7ccccc67)cc5)c5ccc(-c6cccc7ccccc67)cc5)c4)cc3c2)cc1]

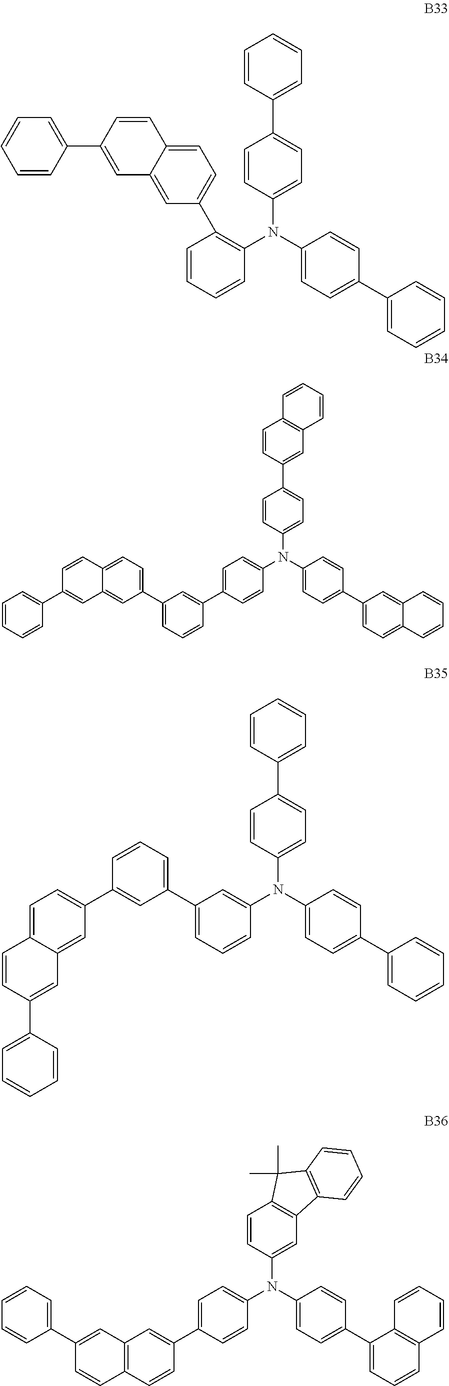 [SMILES: c1ccc(-c2ccc(N(c3ccc(-c4ccccc4)cc3)c3ccccc3-c3ccc4ccc(-c5ccccc5)cc4c3)cc2)cc1 ; c1ccc(-c2ccc3ccc(-c4cccc(-c5ccc(N(c6ccc(-c7ccc8ccccc8c7)cc6)c6ccc(-c7ccc8ccccc8c7)cc6)cc5)c4)cc3c2)cc1 ; c1ccc(-c2ccc(N(c3ccc(-c4ccccc4)cc3)c3cccc(-c4cccc(-c5ccc6ccc(-c7ccccc7)cc6c5)c4)c3)cc2)cc1 ; CC1(C)c2ccccc2-c2cc(N(c3ccc(-c4ccc5ccc(-c6ccccc6)cc5c4)cc3)c3ccc(-c4cccc5ccccc45)cc3)ccc21]

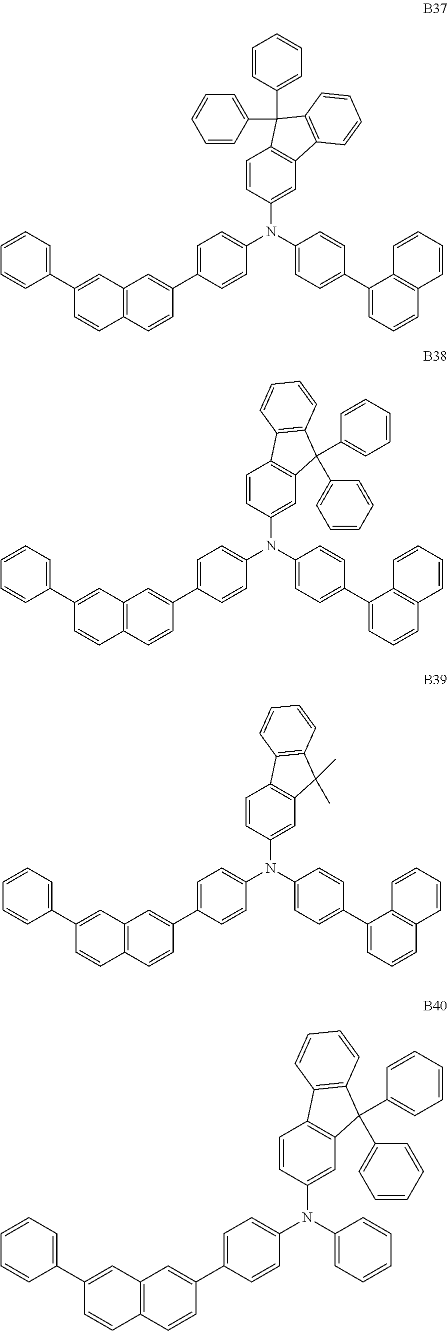 [SMILES: c1ccc(-c2ccc3ccc(-c4ccc(N(c5ccc(-c6cccc7ccccc67)cc5)c5ccc6c(c5)-c5ccccc5C6(c5ccccc5)c5ccccc5)cc4)cc3c2)cc1 ; c1ccc(-c2ccc3ccc(-c4ccc(N(c5ccc(-c6cccc7ccccc67)cc5)c5ccc6c(c5)C(c5ccccc5)(c5ccccc5)c5ccccc5-6)cc4)cc3c2)cc1 ; CC1(C)c2ccccc2-c2ccc(N(c3ccc(-c4ccc5ccc(-c6ccccc6)cc5c4)cc3)c3ccc(-c4cccc5ccccc45)cc3)cc21 ; c1ccc(-c2ccc3ccc(-c4ccc(N(c5ccccc5)c5ccc6c(c5)C(c5ccccc5)(c5ccccc5)c5ccccc5-6)cc4)cc3c2)cc1]

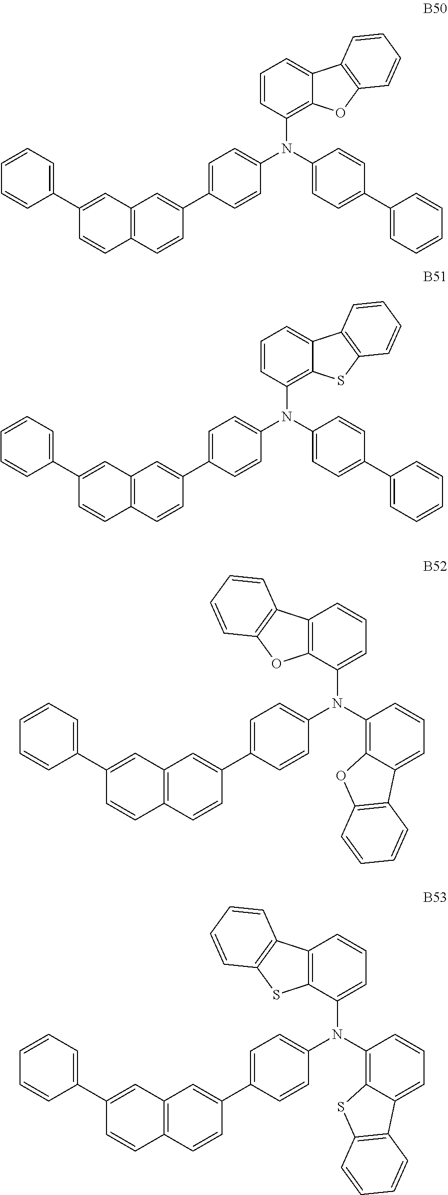 [SMILES: c1ccc(-c2ccc(N(c3ccc(-c4ccc5ccc(-c6ccccc6)cc5c4)cc3)c3cccc4c3oc3ccccc34)cc2)cc1 ; c1ccc(-c2ccc(N(c3ccc(-c4ccc5ccc(-c6ccccc6)cc5c4)cc3)c3cccc4c3sc3ccccc34)cc2)cc1 ; c1ccc(-c2ccc3ccc(-c4ccc(N(c5cccc6c5oc5ccccc56)c5cccc6c5oc5ccccc56)cc4)cc3c2)cc1 ; c1ccc(-c2ccc3ccc(-c4ccc(N(c5cccc6c5sc5ccccc56)c5cccc6c5sc5ccccc56)cc4)cc3c2)cc1]

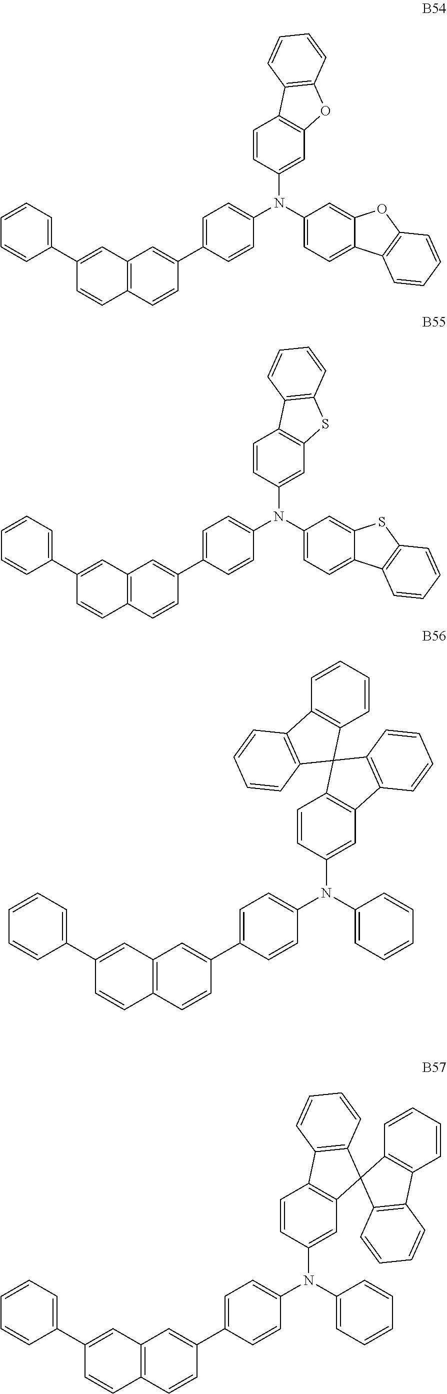 [SMILES: c1ccc(-c2ccc3ccc(-c4ccc(N(c5ccc6c(c5)oc5ccccc56)c5ccc6c(c5)oc5ccccc56)cc4)cc3c2)cc1 ; c1ccc(-c2ccc3ccc(-c4ccc(N(c5ccc6c(c5)sc5ccccc56)c5ccc6c(c5)sc5ccccc56)cc4)cc3c2)cc1 ; c1ccc(-c2ccc3ccc(-c4ccc(N(c5ccccc5)c5ccc6c(c5)-c5ccccc5C65c6ccccc6-c6ccccc65)cc4)cc3c2)cc1 ; c1ccc(-c2ccc3ccc(-c4ccc(N(c5ccccc5)c5ccc6c(c5)C5(c7ccccc7-c7ccccc75)c5ccccc5-6)cc4)cc3c2)cc1]

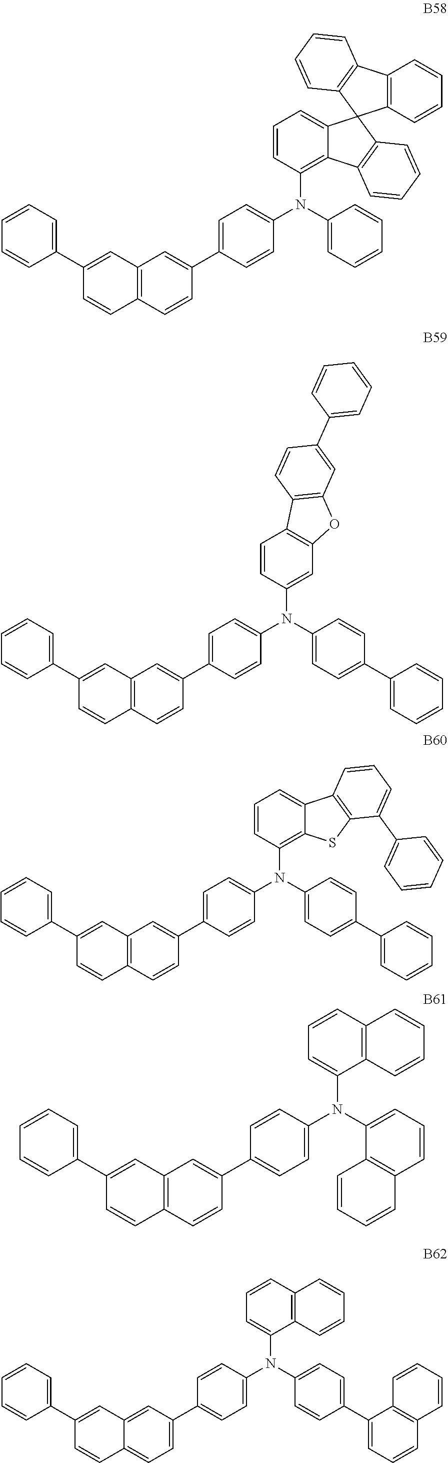

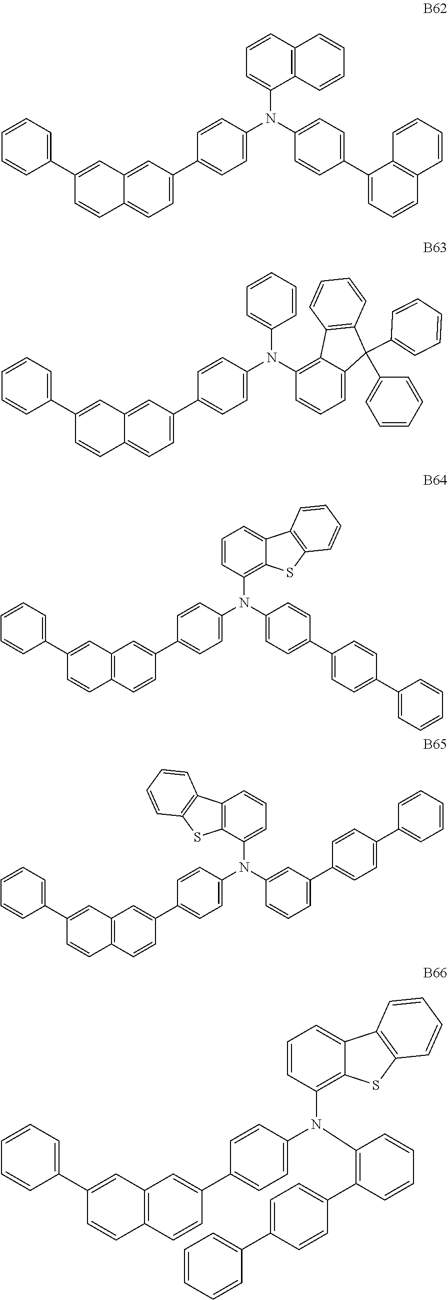 [SMILES: c1ccc(-c2ccc3ccc(-c4ccc(N(c5ccc(-c6cccc7ccccc67)cc5)c5cccc6ccccc56)cc4)cc3c2)cc1 ; c1ccc(-c2ccc3ccc(-c4ccc(N(c5ccccc5)c5cccc6c5-c5ccccc5C6(c5ccccc5)c5ccccc5)cc4)cc3c2)cc1 ; c1ccc(-c2ccc(-c3ccc(N(c4ccc(-c5ccc6ccc(-c7ccccc7)cc6c5)cc4)c4cccc5c4sc4ccccc45)cc3)cc2)cc1 ; c1ccc(-c2ccc(-c3cccc(N(c4ccc(-c5ccc6ccc(-c7ccccc7)cc6c5)cc4)c4cccc5c4sc4ccccc45)c3)cc2)cc1 ; c1ccc(-c2ccc(-c3ccccc3N(c3ccc(-c4ccc5ccc(-c6ccccc6)cc5c4)cc3)c3cccc4c3sc3ccccc34)cc2)cc1]

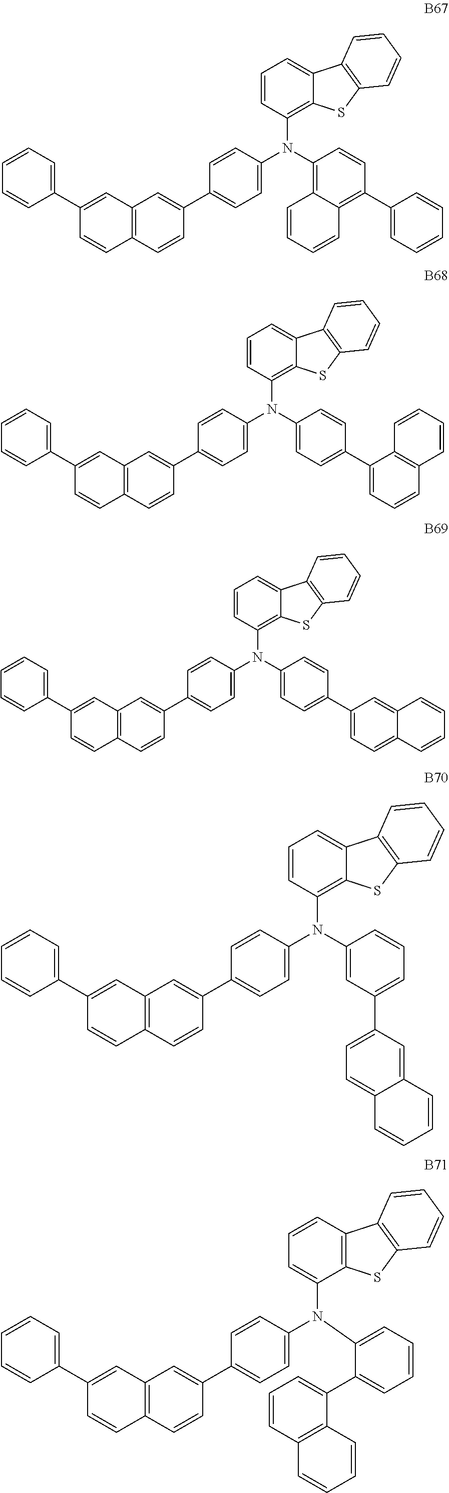 [SMILES: c1ccc(-c2ccc3ccc(-c4ccc(N(c5ccc(-c6ccccc6)c6ccccc56)c5cccc6c5sc5ccccc56)cc4)cc3c2)cc1 ; c1ccc(-c2ccc3ccc(-c4ccc(N(c5ccc(-c6cccc7ccccc67)cc5)c5cccc6c5sc5ccccc56)cc4)cc3c2)cc1 ; c1ccc(-c2ccc3ccc(-c4ccc(N(c5ccc(-c6ccc7ccccc7c6)cc5)c5cccc6c5sc5ccccc56)cc4)cc3c2)cc1 ; c1ccc(-c2ccc3ccc(-c4ccc(N(c5cccc(-c6ccc7ccccc7c6)c5)c5cccc6c5sc5ccccc56)cc4)cc3c2)cc1 ; c1ccc(-c2ccc3ccc(-c4ccc(N(c5ccccc5-c5cccc6ccccc56)c5cccc6c5sc5ccccc56)cc4)cc3c2)cc1]

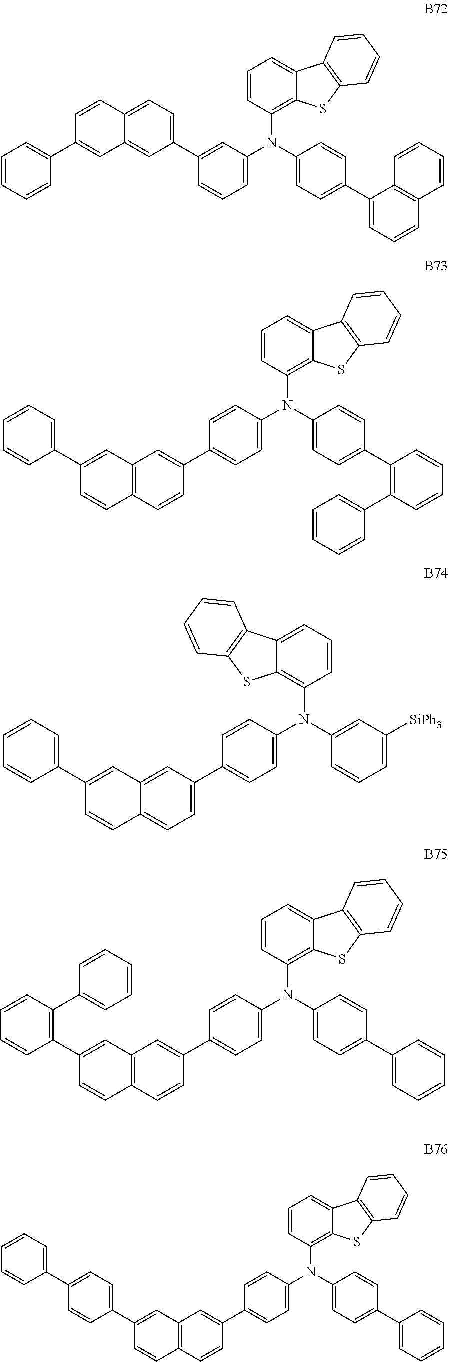

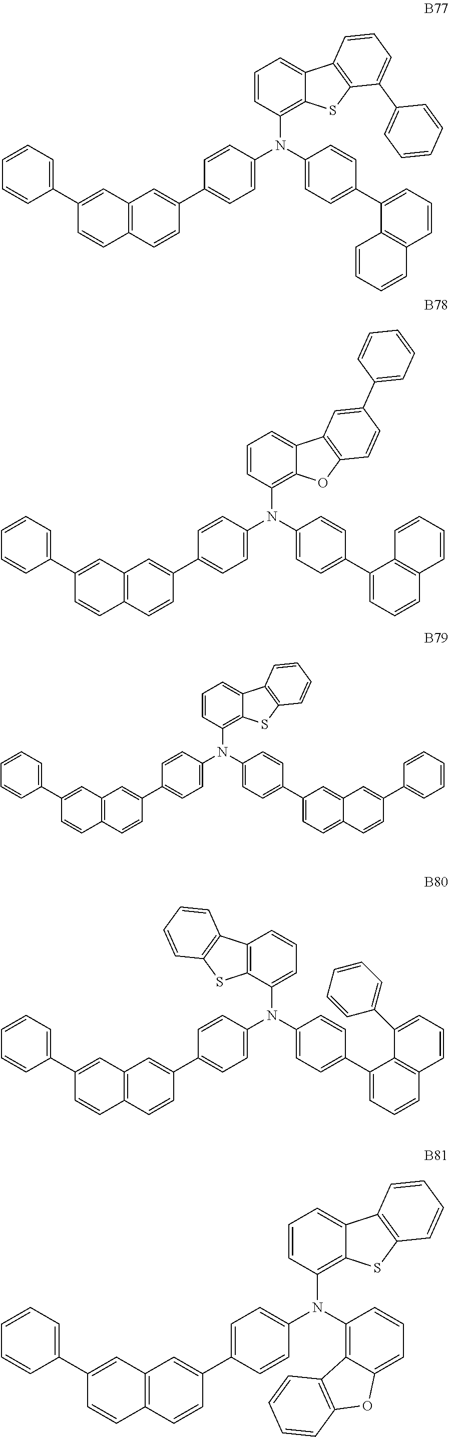 [SMILES: c1ccc(-c2ccc3ccc(-c4ccc(N(c5ccc(-c6cccc7ccccc67)cc5)c5cccc6c5sc5c(-c7ccccc7)cccc56)cc4)cc3c2)cc1 ; c1ccc(-c2ccc3ccc(-c4ccc(N(c5ccc(-c6cccc7ccccc67)cc5)c5cccc6c5oc5ccc(-c7ccccc7)cc56)cc4)cc3c2)cc1 ; c1ccc(-c2ccc3ccc(-c4ccc(N(c5ccc(-c6ccc7ccc(-c8ccccc8)cc7c6)cc5)c5cccc6c5sc5ccccc56)cc4)cc3c2)cc1 ; c1ccc(-c2ccc3ccc(-c4ccc(N(c5ccc(-c6cccc7cccc(-c8ccccc8)c67)cc5)c5cccc6c5sc5ccccc56)cc4)cc3c2)cc1 ; c1ccc(-c2ccc3ccc(-c4ccc(N(c5cccc6c5sc5ccccc56)c5cccc6oc7ccccc7c56)cc4)cc3c2)cc1]

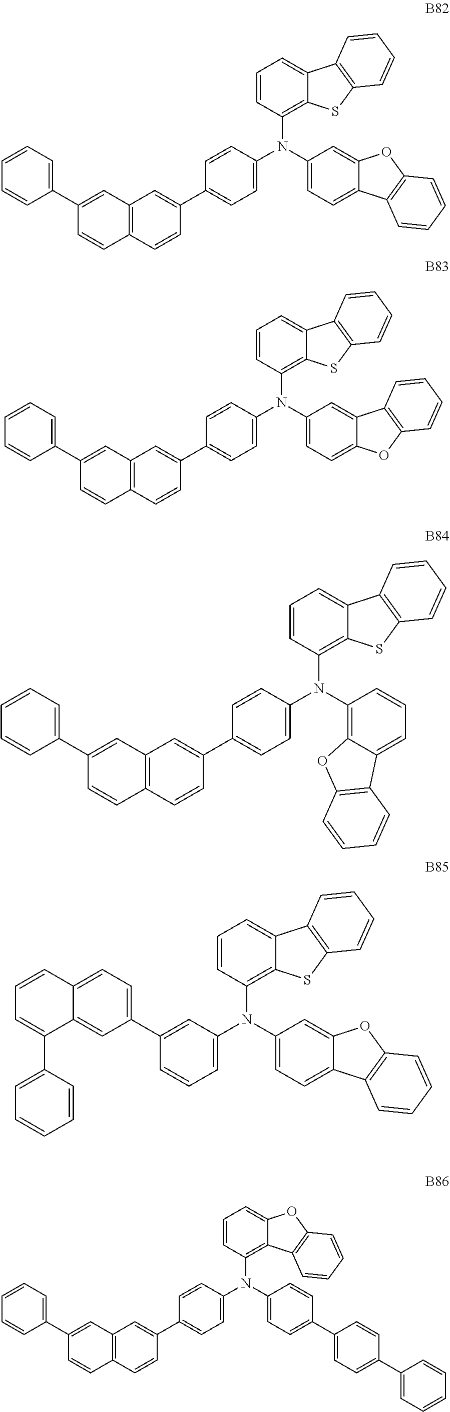 [SMILES: c1ccc(-c2ccc3ccc(-c4ccc(N(c5ccc6c(c5)oc5ccccc56)c5cccc6c5sc5ccccc56)cc4)cc3c2)cc1 ; c1ccc(-c2ccc3ccc(-c4ccc(N(c5ccc6oc7ccccc7c6c5)c5cccc6c5sc5ccccc56)cc4)cc3c2)cc1 ; c1ccc(-c2ccc3ccc(-c4ccc(N(c5cccc6c5oc5ccccc56)c5cccc6c5sc5ccccc56)cc4)cc3c2)cc1 ; c1ccc(-c2cccc3ccc(-c4cccc(N(c5ccc6c(c5)oc5ccccc56)c5cccc6c5sc5ccccc56)c4)cc23)cc1 ; c1ccc(-c2ccc(-c3ccc(N(c4ccc(-c5ccc6ccc(-c7ccccc7)cc6c5)cc4)c4cccc5oc6ccccc6c45)cc3)cc2)cc1]

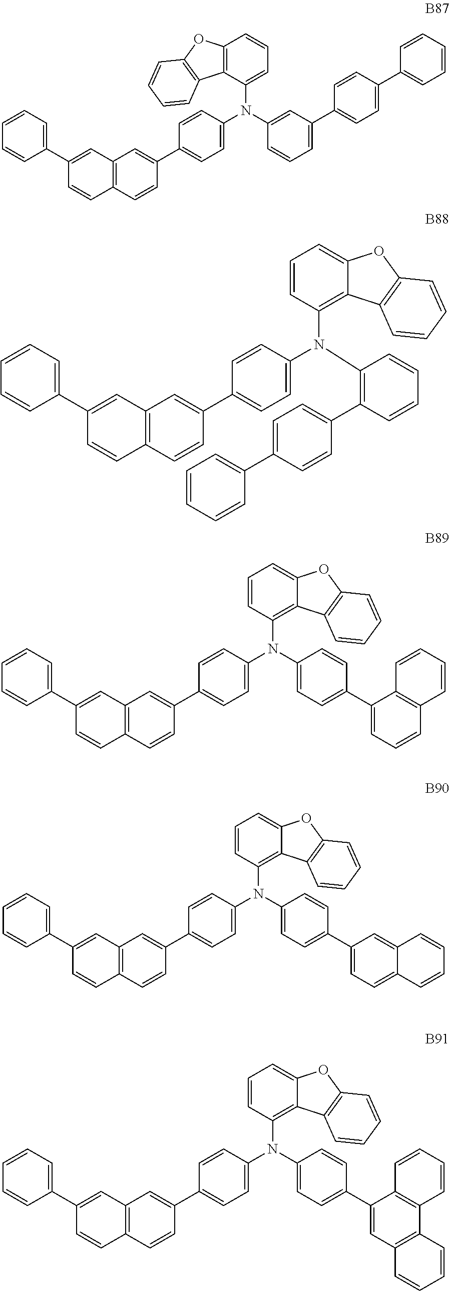 [SMILES: c1ccc(-c2ccc(-c3cccc(N(c4ccc(-c5ccc6ccc(-c7ccccc7)cc6c5)cc4)c4cccc5oc6ccccc6c45)c3)cc2)cc1 ; c1ccc(-c2ccc(-c3ccccc3N(c3ccc(-c4ccc5ccc(-c6ccccc6)cc5c4)cc3)c3cccc4oc5ccccc5c34)cc2)cc1 ; c1ccc(-c2ccc3ccc(-c4ccc(N(c5ccc(-c6cccc7ccccc67)cc5)c5cccc6oc7ccccc7c56)cc4)cc3c2)cc1 ; c1ccc(-c2ccc3ccc(-c4ccc(N(c5ccc(-c6ccc7ccccc7c6)cc5)c5cccc6oc7ccccc7c56)cc4)cc3c2)cc1 ; c1ccc(-c2ccc3ccc(-c4ccc(N(c5ccc(-c6cc7ccccc7c7ccccc67)cc5)c5cccc6oc7ccccc7c56)cc4)cc3c2)cc1]

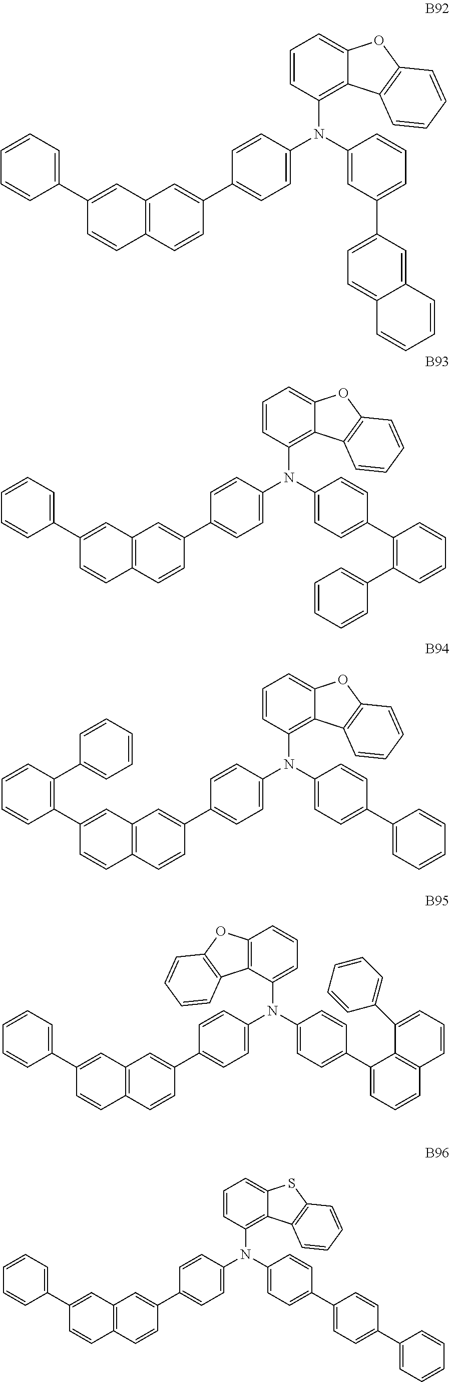 [SMILES: c1ccc(-c2ccc3ccc(-c4ccc(N(c5cccc(-c6ccc7ccccc7c6)c5)c5cccc6oc7ccccc7c56)cc4)cc3c2)cc1 ; c1ccc(-c2ccc3ccc(-c4ccc(N(c5ccc(-c6ccccc6-c6ccccc6)cc5)c5cccc6oc7ccccc7c56)cc4)cc3c2)cc1 ; c1ccc(-c2ccc(N(c3ccc(-c4ccc5ccc(-c6ccccc6-c6ccccc6)cc5c4)cc3)c3cccc4oc5ccccc5c34)cc2)cc1 ; c1ccc(-c2ccc3ccc(-c4ccc(N(c5ccc(-c6cccc7cccc(-c8ccccc8)c67)cc5)c5cccc6oc7ccccc7c56)cc4)cc3c2)cc1 ; c1ccc(-c2ccc(-c3ccc(N(c4ccc(-c5ccc6ccc(-c7ccccc7)cc6c5)cc4)c4cccc5sc6ccccc6c45)cc3)cc2)cc1]

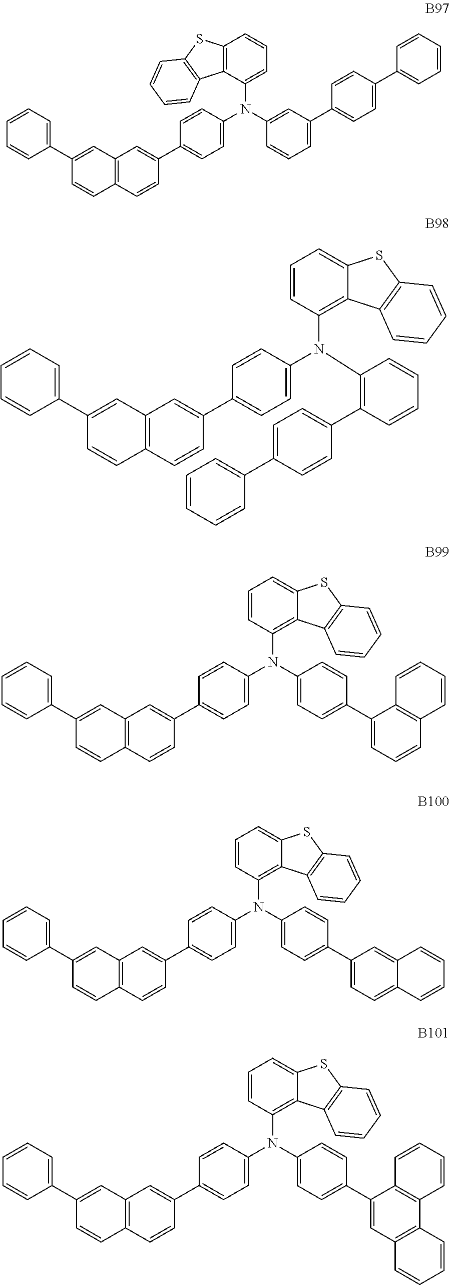 [SMILES: c1ccc(-c2ccc(-c3cccc(N(c4ccc(-c5ccc6ccc(-c7ccccc7)cc6c5)cc4)c4cccc5sc6ccccc6c45)c3)cc2)cc1 ; c1ccc(-c2ccc(-c3ccccc3N(c3ccc(-c4ccc5ccc(-c6ccccc6)cc5c4)cc3)c3cccc4sc5ccccc5c34)cc2)cc1 ; c1ccc(-c2ccc3ccc(-c4ccc(N(c5ccc(-c6cccc7ccccc67)cc5)c5cccc6sc7ccccc7c56)cc4)cc3c2)cc1 ; c1ccc(-c2ccc3ccc(-c4ccc(N(c5ccc(-c6ccc7ccccc7c6)cc5)c5cccc6sc7ccccc7c56)cc4)cc3c2)cc1 ; c1ccc(-c2ccc3ccc(-c4ccc(N(c5ccc(-c6cc7ccccc7c7ccccc67)cc5)c5cccc6sc7ccccc7c56)cc4)cc3c2)cc1]

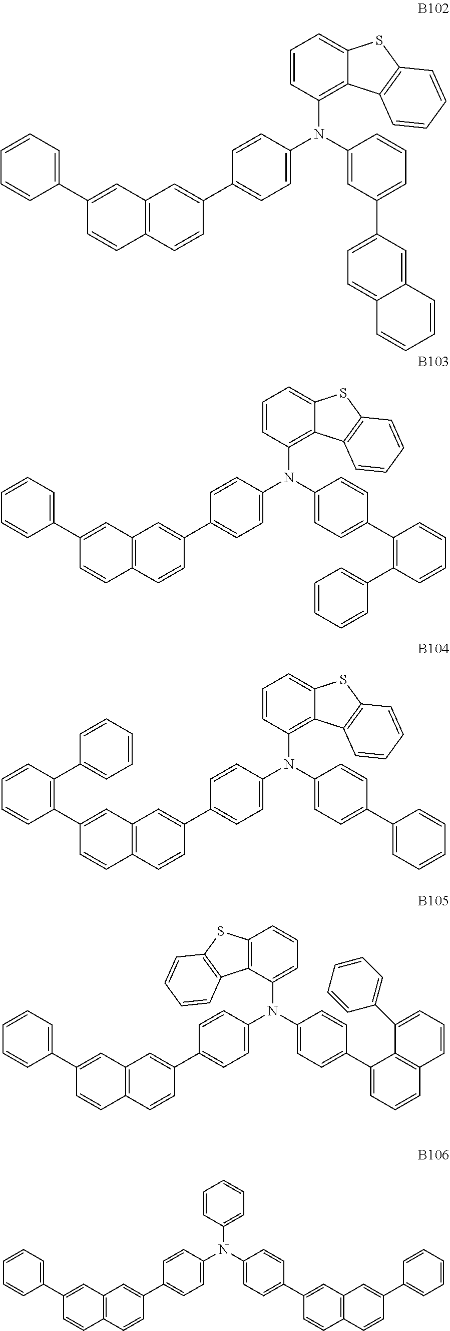 [SMILES: c1ccc(-c2ccc3ccc(-c4ccc(N(c5cccc(-c6ccc7ccccc7c6)c5)c5cccc6sc7ccccc7c56)cc4)cc3c2)cc1 ; c1ccc(-c2ccc3ccc(-c4ccc(N(c5ccc(-c6ccccc6-c6ccccc6)cc5)c5cccc6sc7ccccc7c56)cc4)cc3c2)cc1 ; c1ccc(-c2ccc(N(c3ccc(-c4ccc5ccc(-c6ccccc6-c6ccccc6)cc5c4)cc3)c3cccc4sc5ccccc5c34)cc2)cc1 ; c1ccc(-c2ccc3ccc(-c4ccc(N(c5ccc(-c6cccc7cccc(-c8ccccc8)c67)cc5)c5cccc6sc7ccccc7c56)cc4)cc3c2)cc1 ; c1ccc(-c2ccc3ccc(-c4ccc(N(c5ccccc5)c5ccc(-c6ccc7ccc(-c8ccccc8)cc7c6)cc5)cc4)cc3c2)cc1]

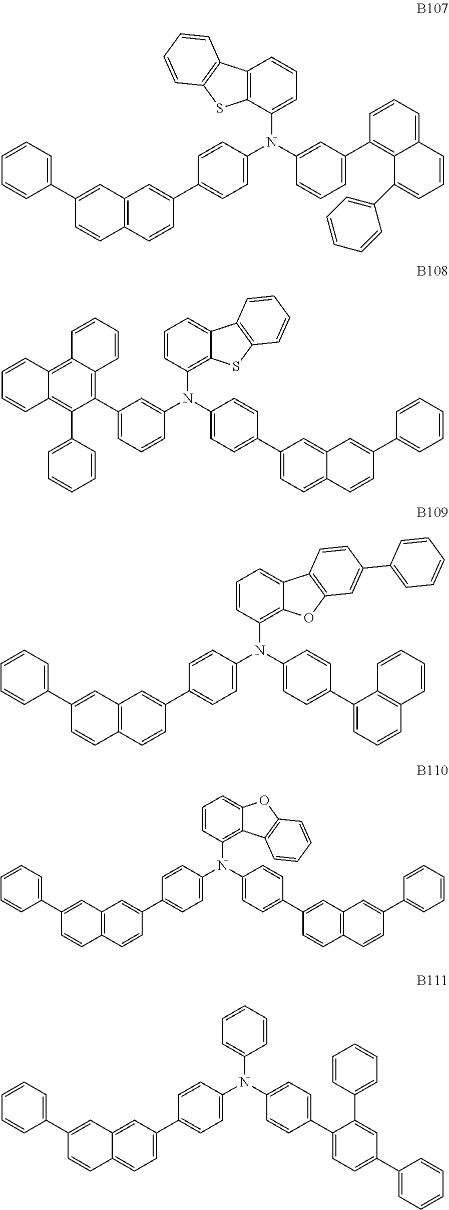 [SMILES: c1ccc(-c2ccc3ccc(-c4ccc(N(c5cccc(-c6cccc7cccc(-c8ccccc8)c67)c5)c5cccc6c5sc5ccccc56)cc4)cc3c2)cc1 ; c1ccc(-c2ccc3ccc(-c4ccc(N(c5cccc(-c6c(-c7ccccc7)c7ccccc7c7ccccc67)c5)c5cccc6c5sc5ccccc56)cc4)cc3c2)cc1 ; c1ccc(-c2ccc3ccc(-c4ccc(N(c5ccc(-c6cccc7ccccc67)cc5)c5cccc6c5oc5cc(-c7ccccc7)ccc56)cc4)cc3c2)cc1 ; c1ccc(-c2ccc3ccc(-c4ccc(N(c5ccc(-c6ccc7ccc(-c8ccccc8)cc7c6)cc5)c5cccc6oc7ccccc7c56)cc4)cc3c2)cc1 ; c1ccc(-c2ccc(-c3ccc(N(c4ccccc4)c4ccc(-c5ccc6ccc(-c7ccccc7)cc6c5)cc4)cc3)c(-c3ccccc3)c2)cc1]

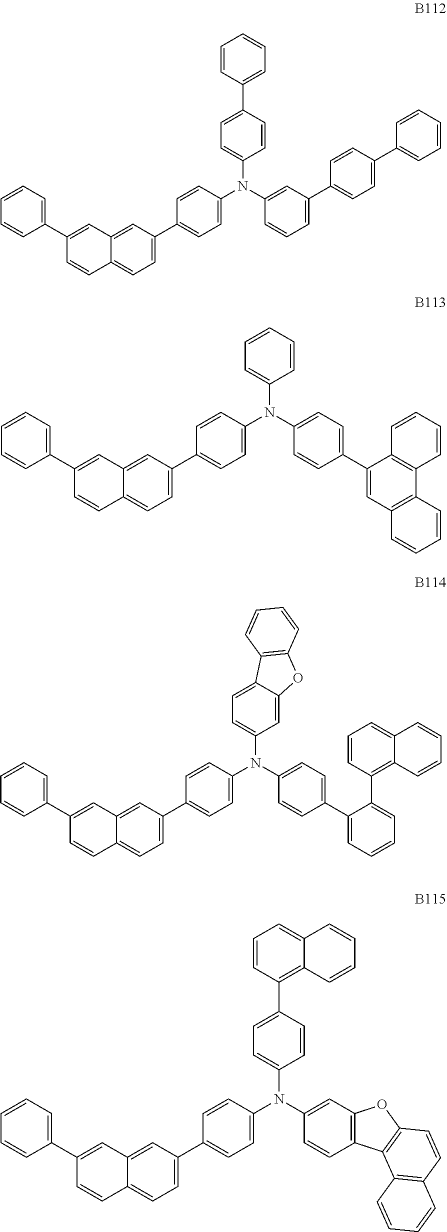 [SMILES: c1ccc(-c2ccc(-c3cccc(N(c4ccc(-c5ccccc5)cc4)c4ccc(-c5ccc6ccc(-c7ccccc7)cc6c5)cc4)c3)cc2)cc1 ; c1ccc(-c2ccc3ccc(-c4ccc(N(c5ccccc5)c5ccc(-c6cc7ccccc7c7ccccc67)cc5)cc4)cc3c2)cc1 ; c1ccc(-c2ccc3ccc(-c4ccc(N(c5ccc(-c6ccccc6-c6cccc7ccccc67)cc5)c5ccc6c(c5)oc5ccccc56)cc4)cc3c2)cc1 ; c1ccc(-c2ccc3ccc(-c4ccc(N(c5ccc(-c6cccc7ccccc67)cc5)c5ccc6c(c5)oc5ccc7ccccc7c56)cc4)cc3c2)cc1]

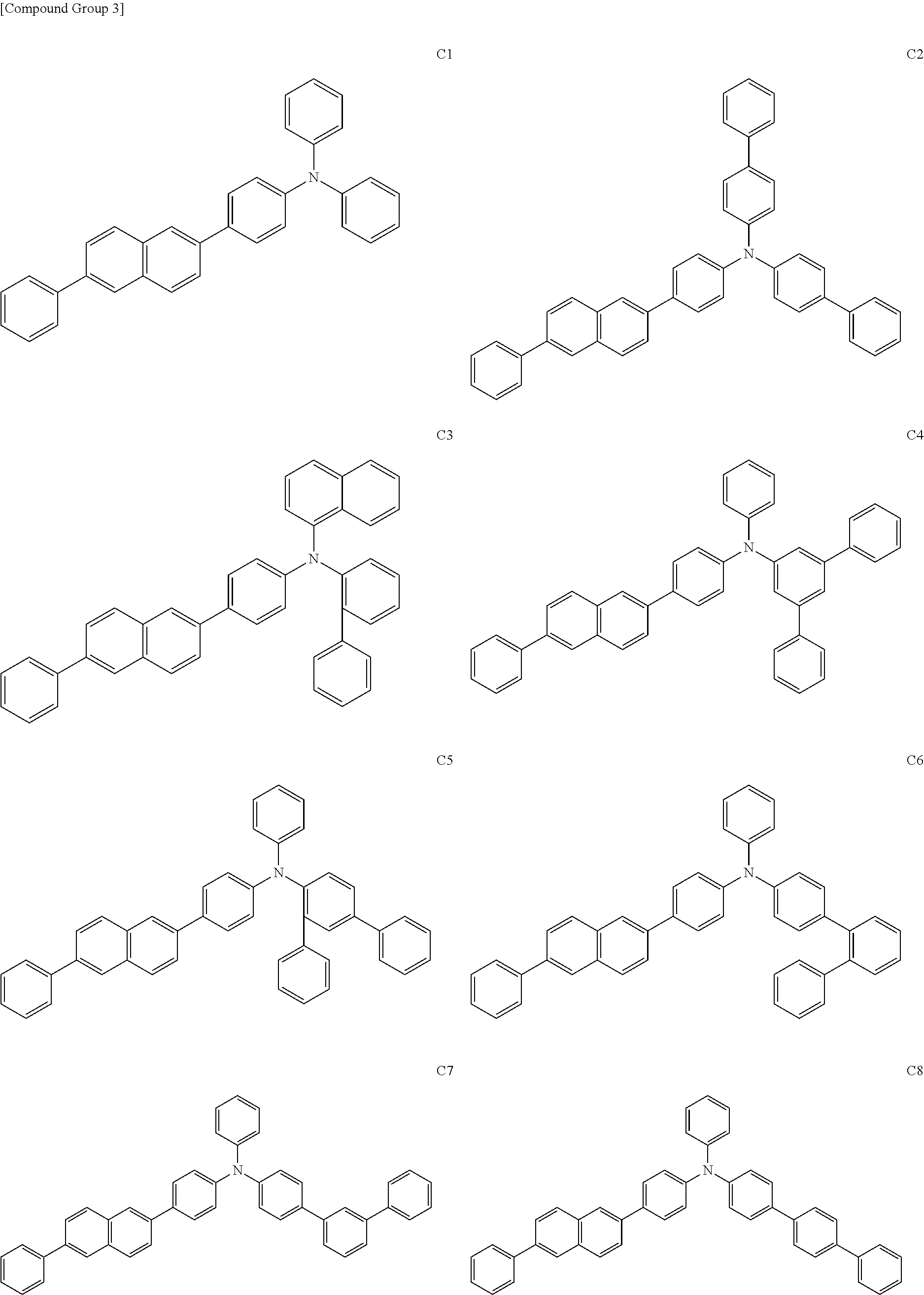

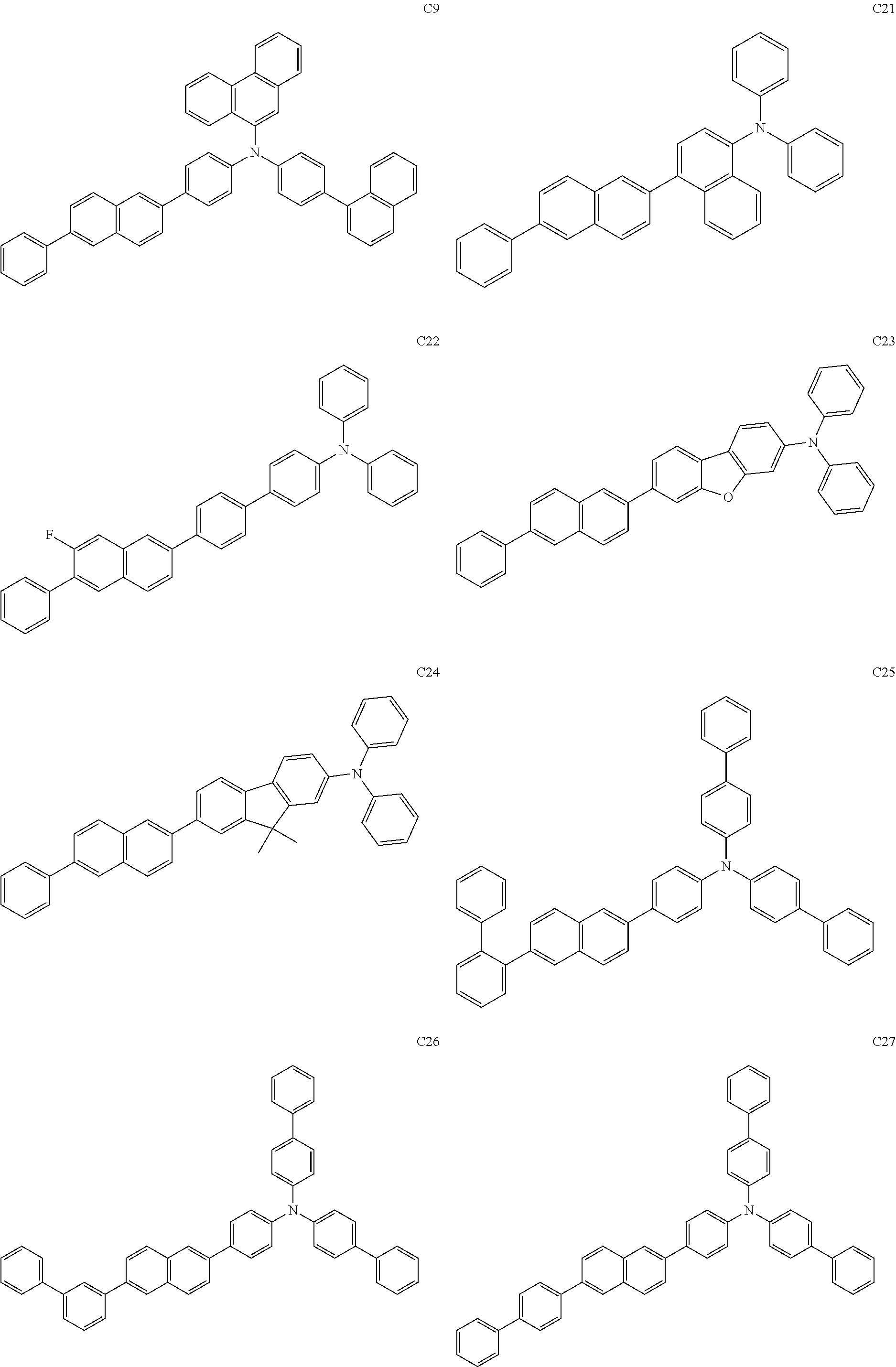

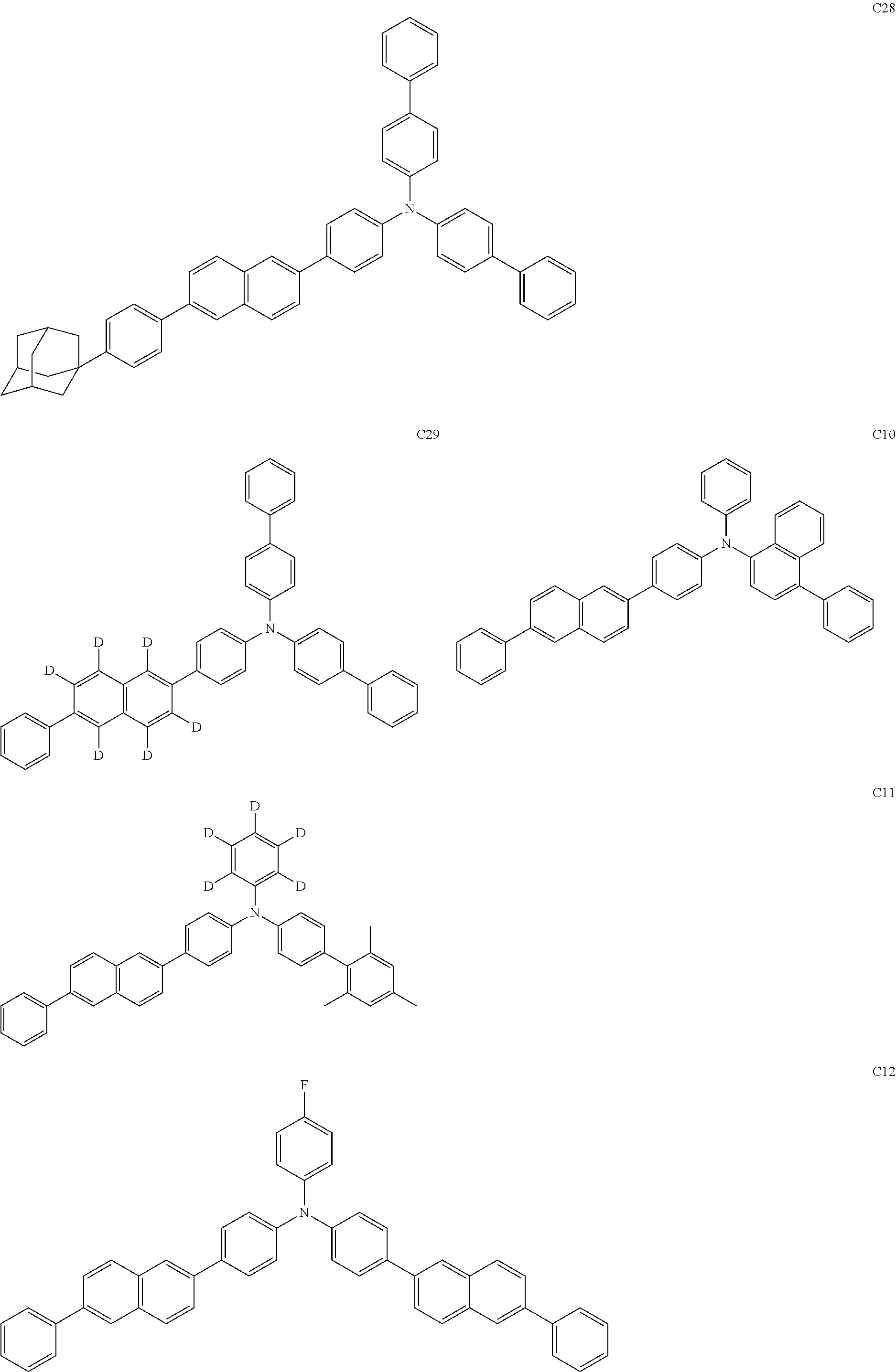 [SMILES: c1ccc(-c2ccc(N(c3ccc(-c4ccccc4)cc3)c3ccc(-c4ccc5cc(-c6ccc(C78CC9CC(CC(C9)C7)C8)cc6)ccc5c4)cc3)cc2)cc1 ; [2H]c1c(-c2ccc(N(c3ccc(-c4ccccc4)cc3)c3ccc(-c4ccccc4)cc3)cc2)c([2H])c2c([2H])c([2H])c(-c3ccccc3)c([2H])c2c1[2H] ; c1ccc(-c2ccc3cc(-c4ccc(N(c5ccccc5)c5ccc(-c6ccccc6)c6ccccc56)cc4)ccc3c2)cc1 ; [2H]c1c([2H])c([2H])c(N(c2ccc(-c3ccc4cc(-c5ccccc5)ccc4c3)cc2)c2ccc(-c3c(C)cc(C)cc3C)cc2)c([2H])c1[2H] ; Fc1ccc(N(c2ccc(-c3ccc4cc(-c5ccccc5)ccc4c3)cc2)c2ccc(-c3ccc4cc(-c5ccccc5)ccc4c3)cc2)cc1]

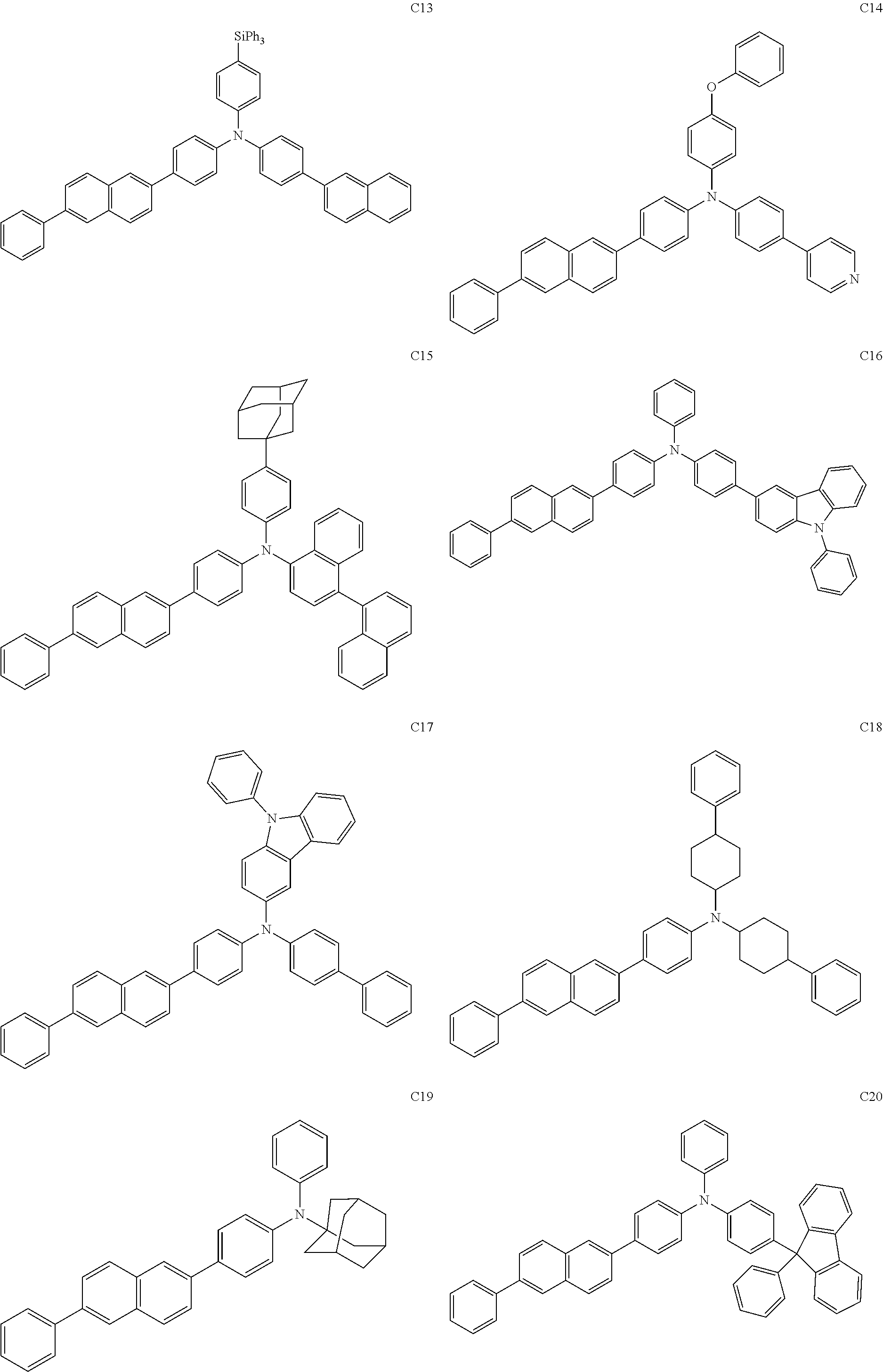

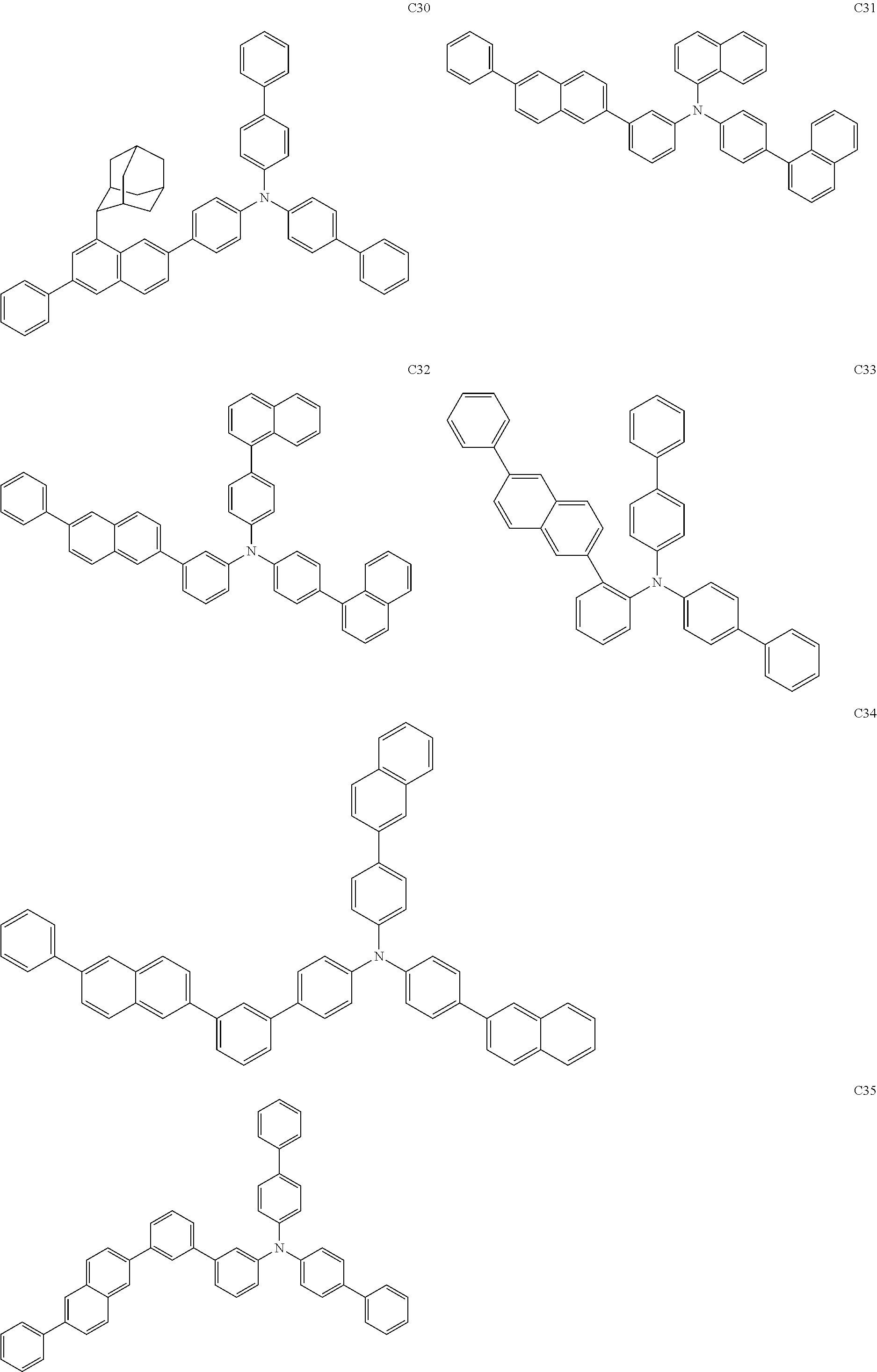

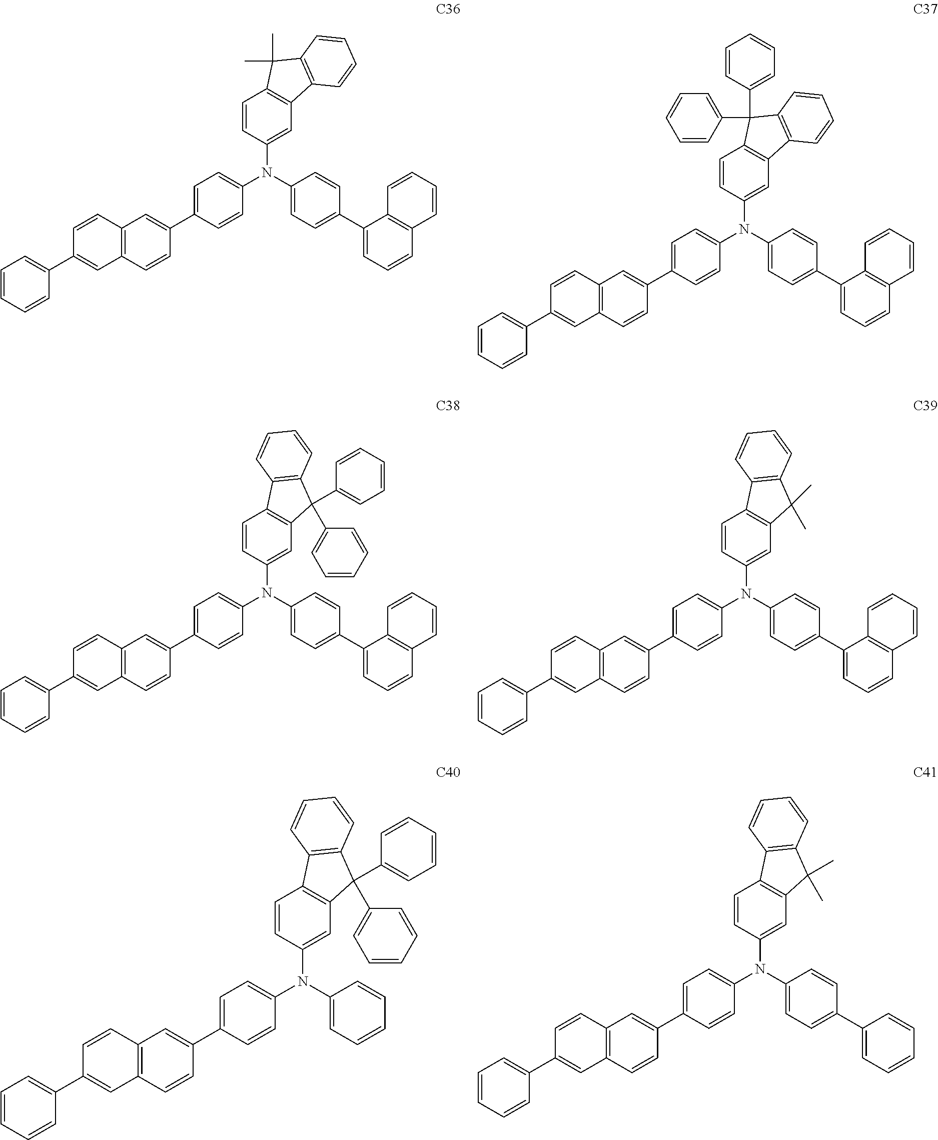

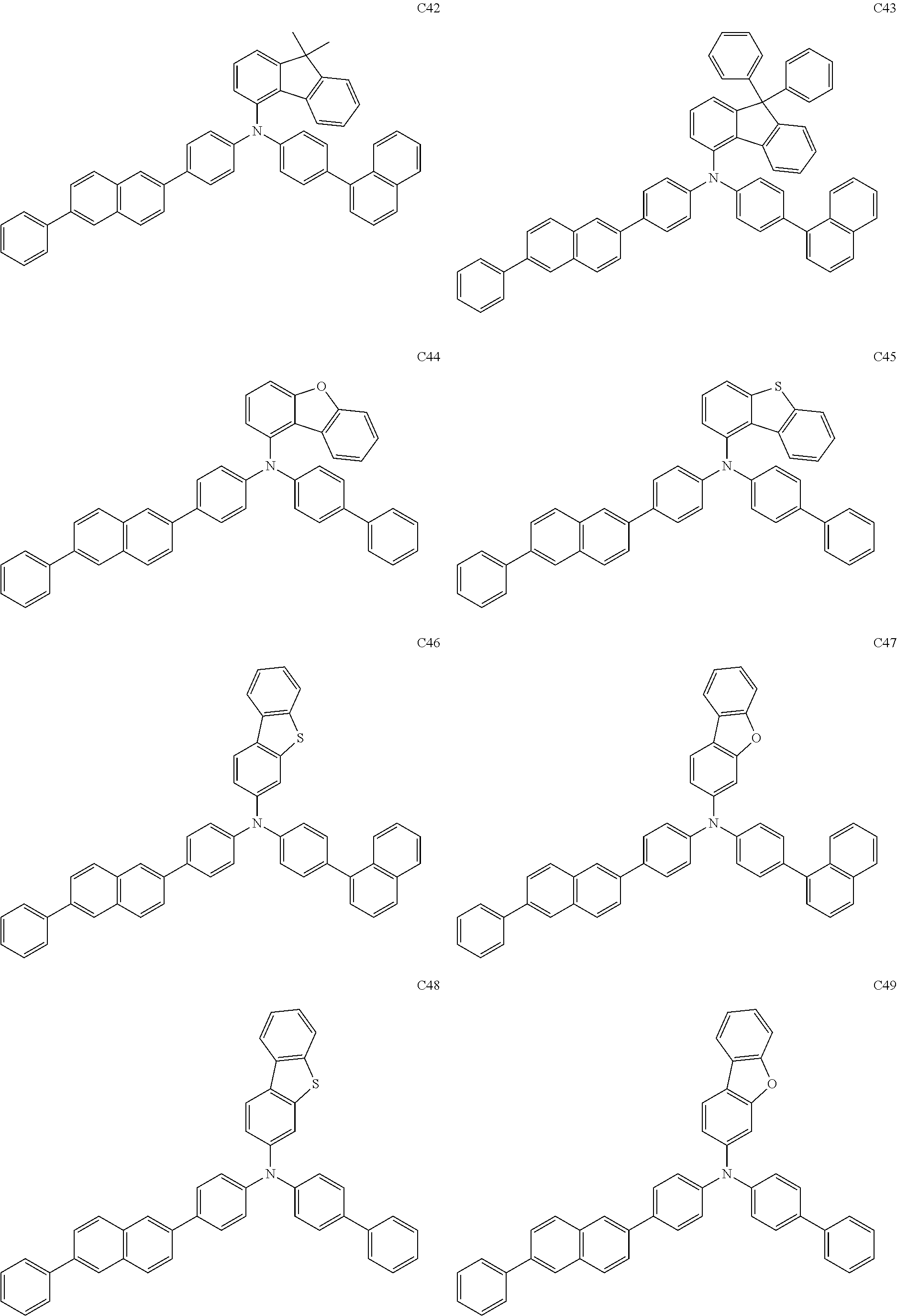

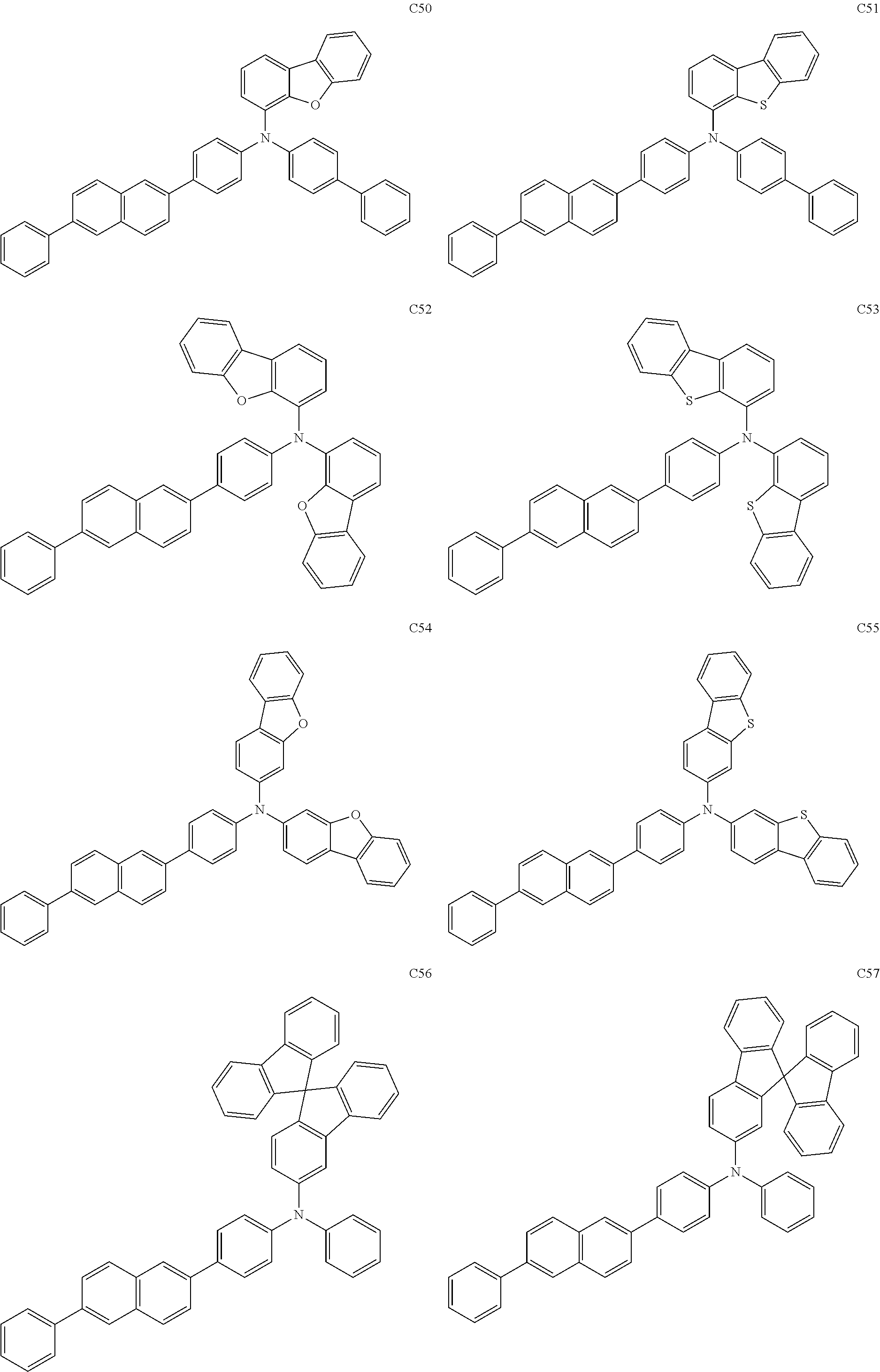

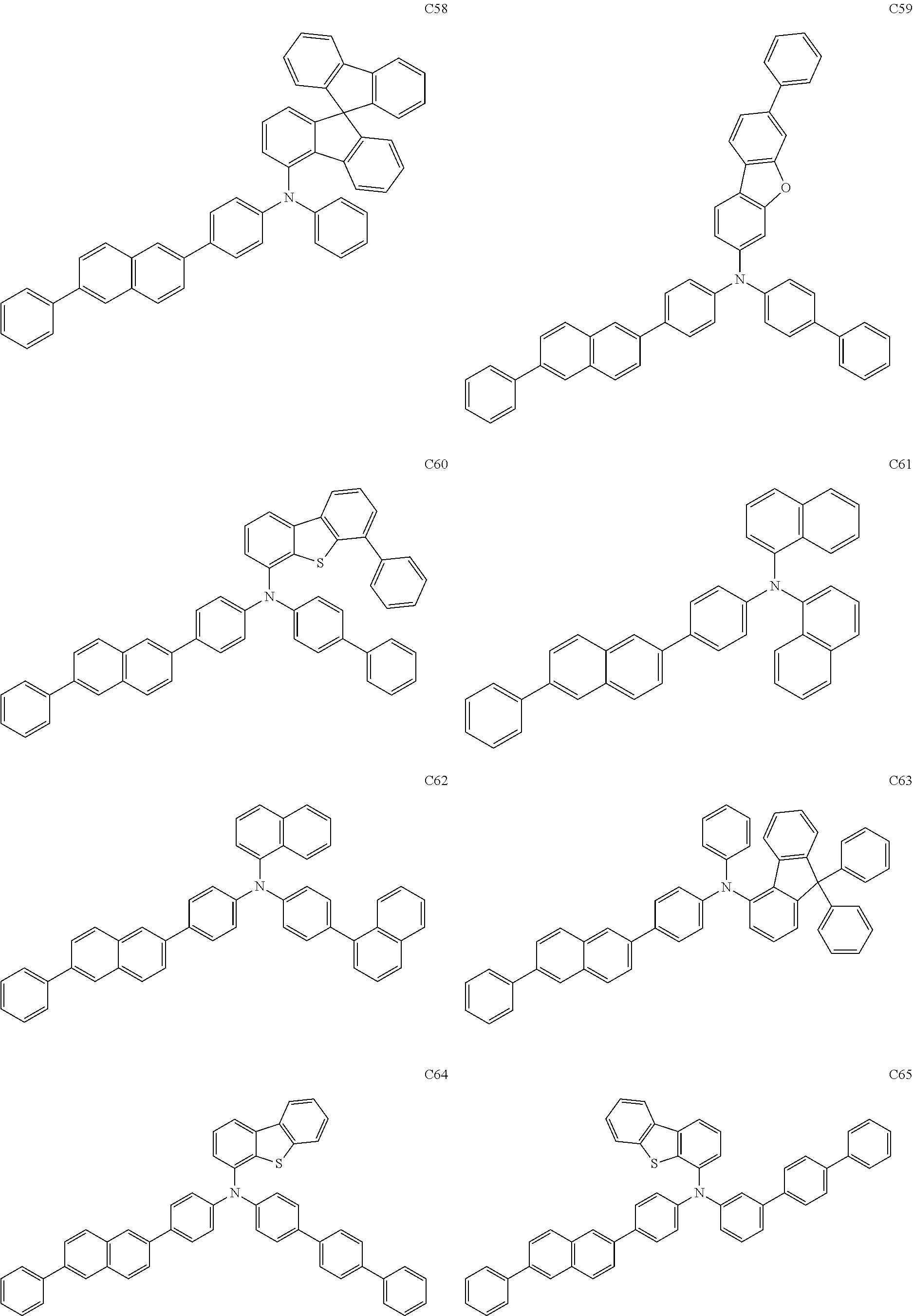

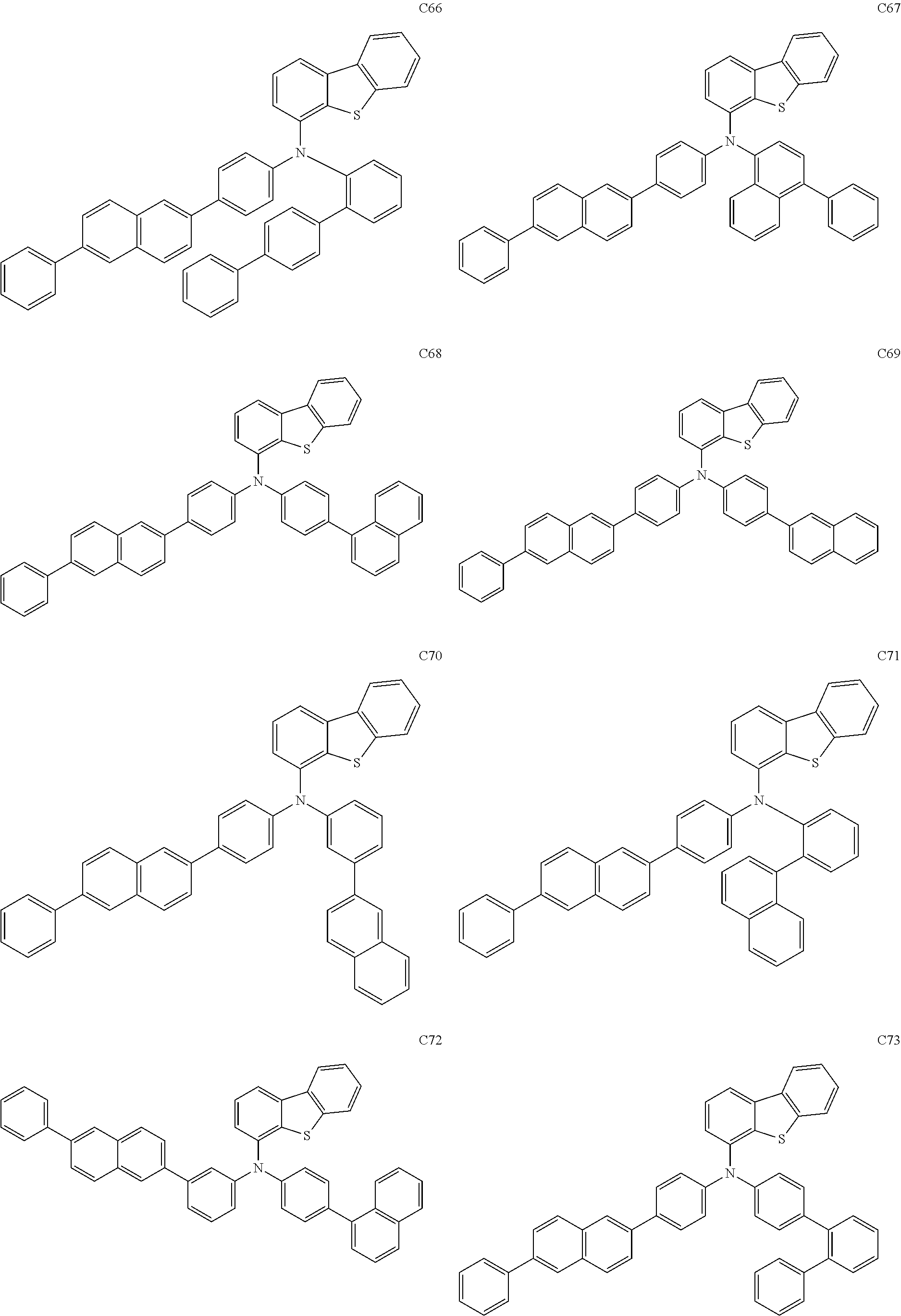

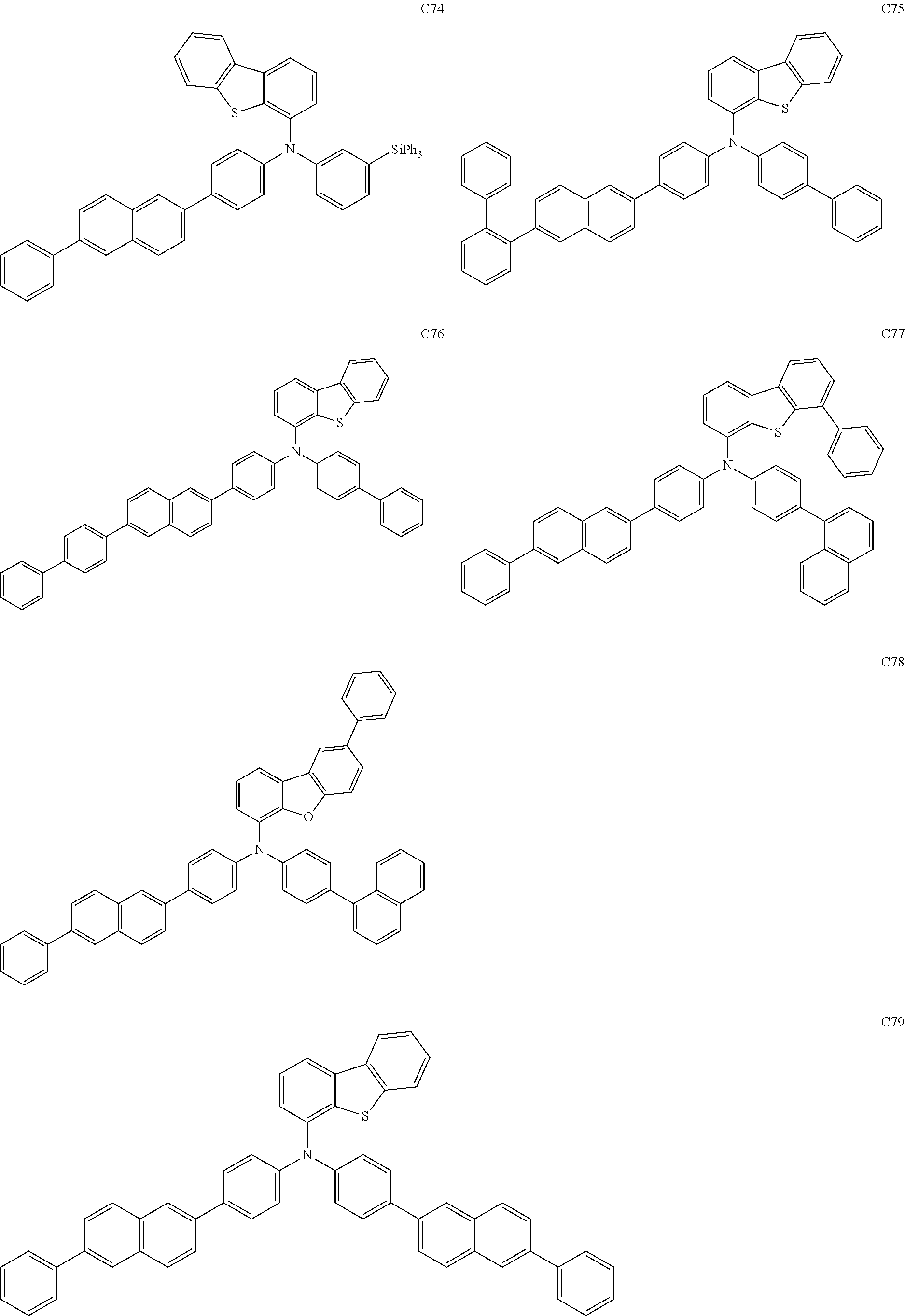

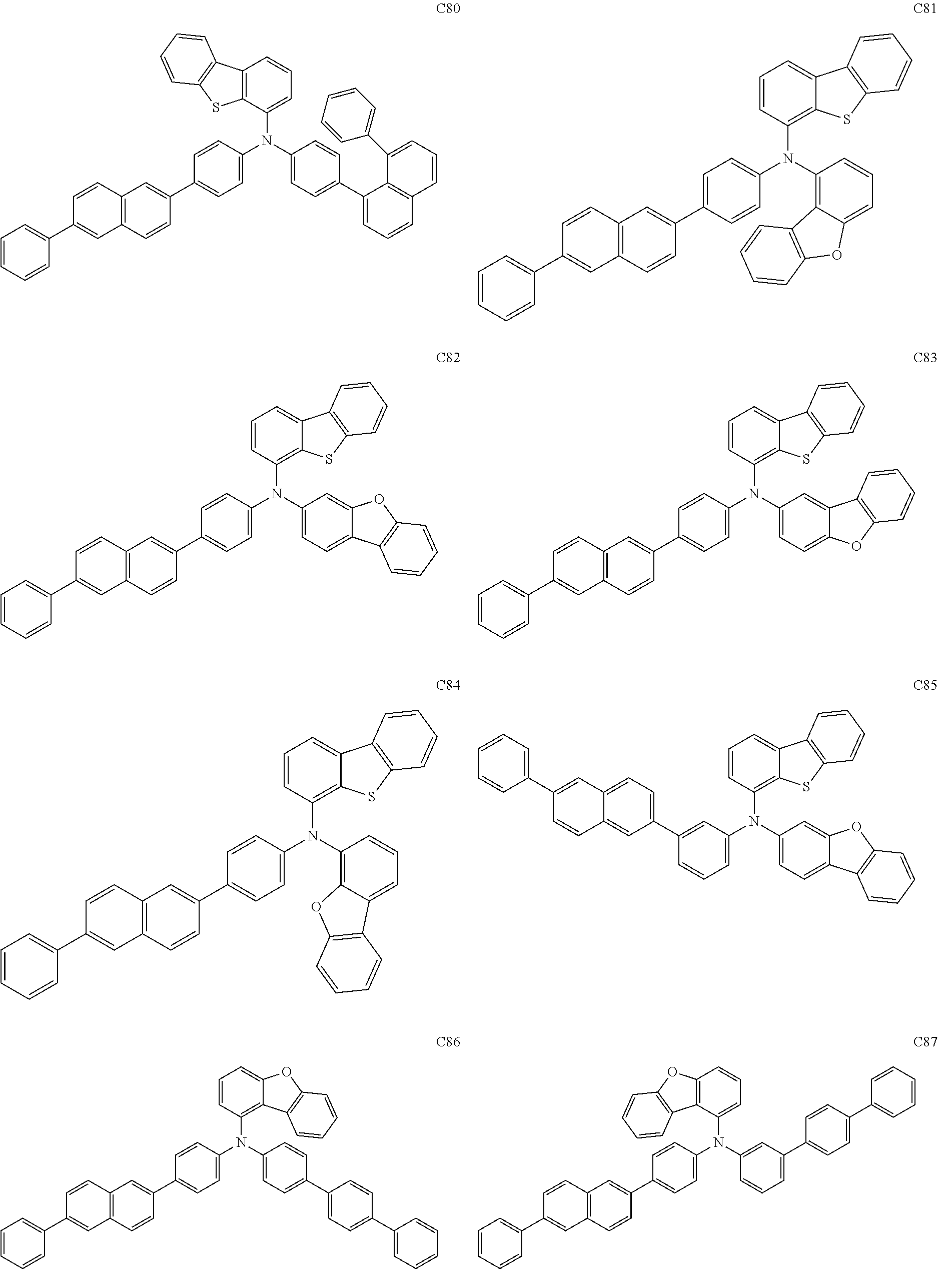

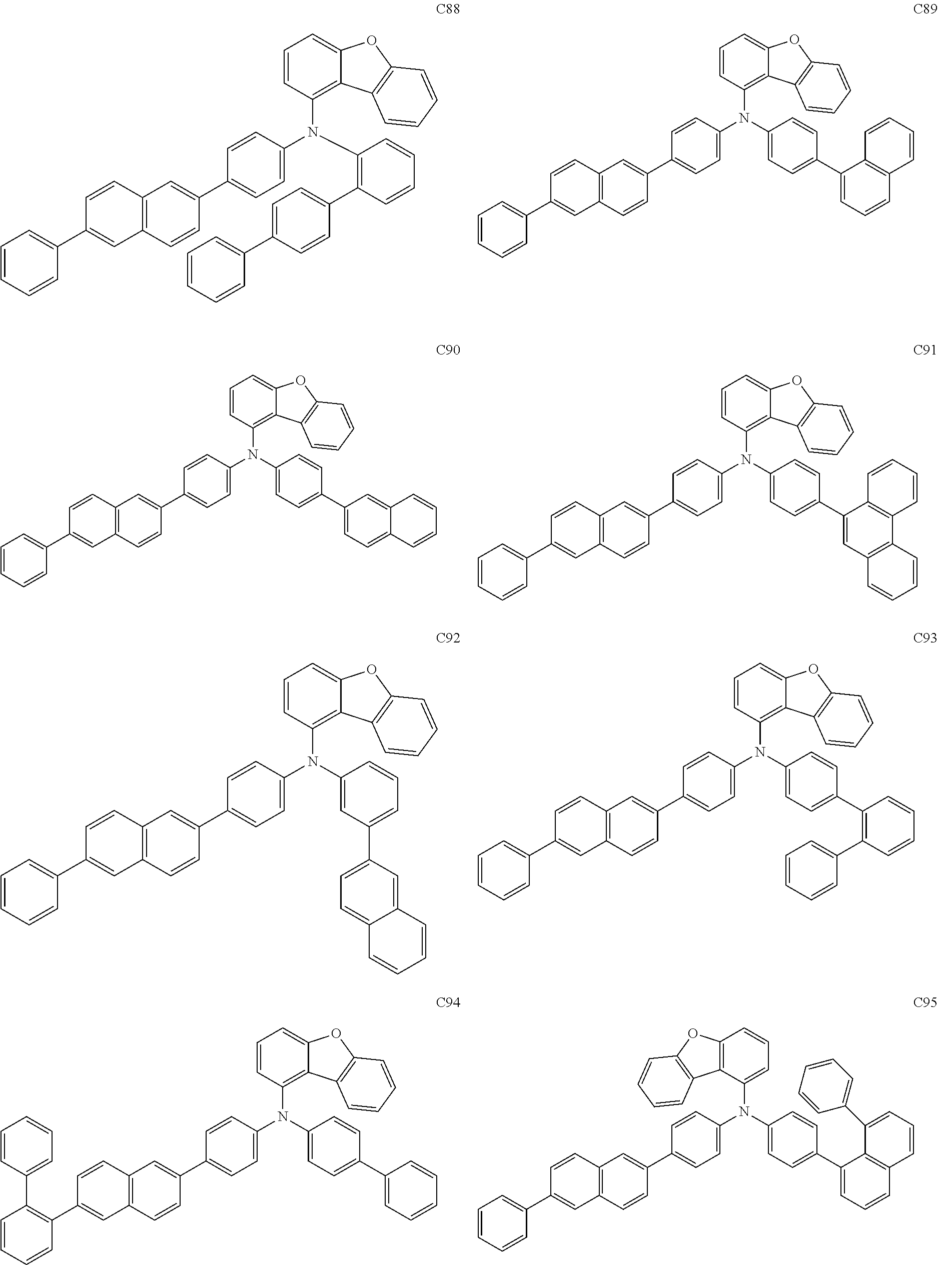

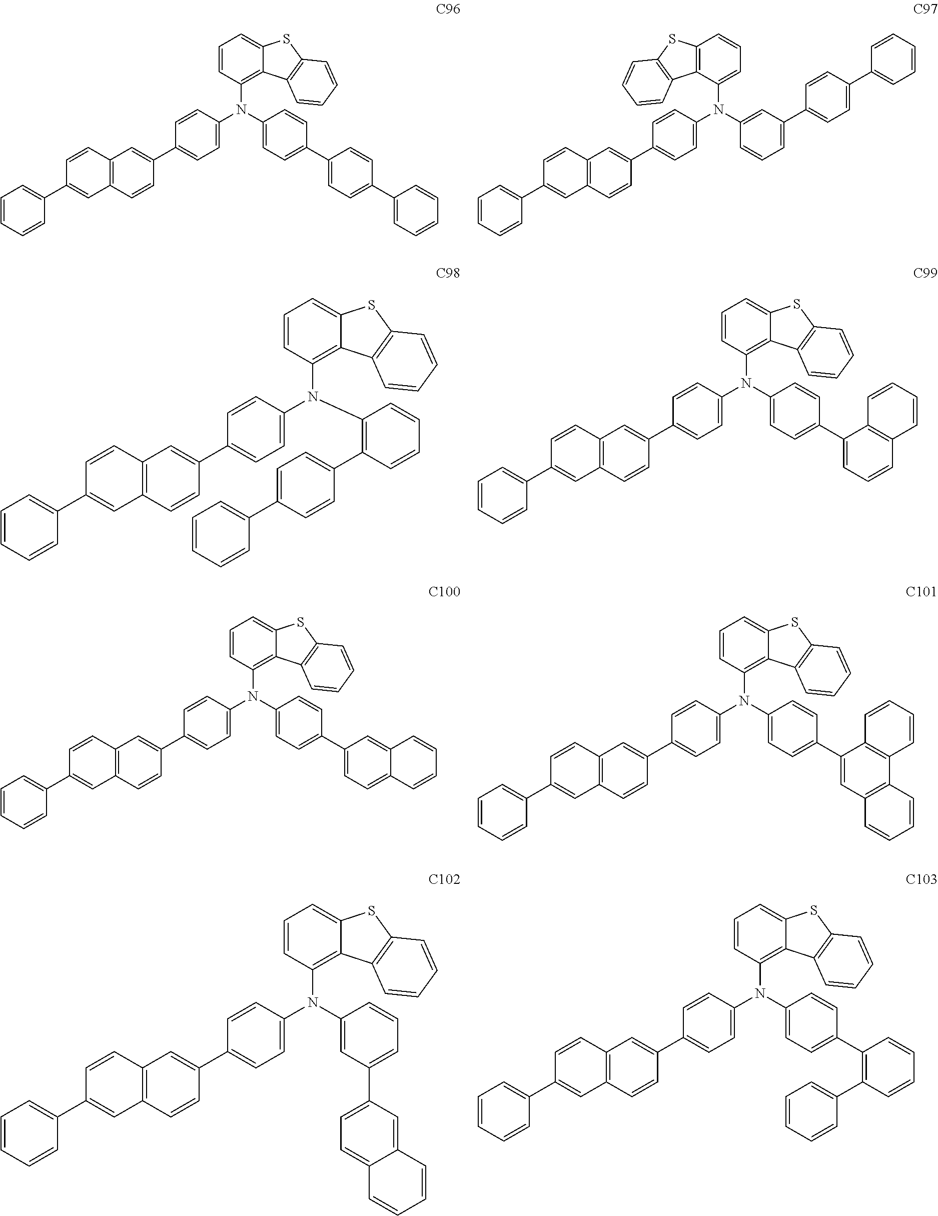

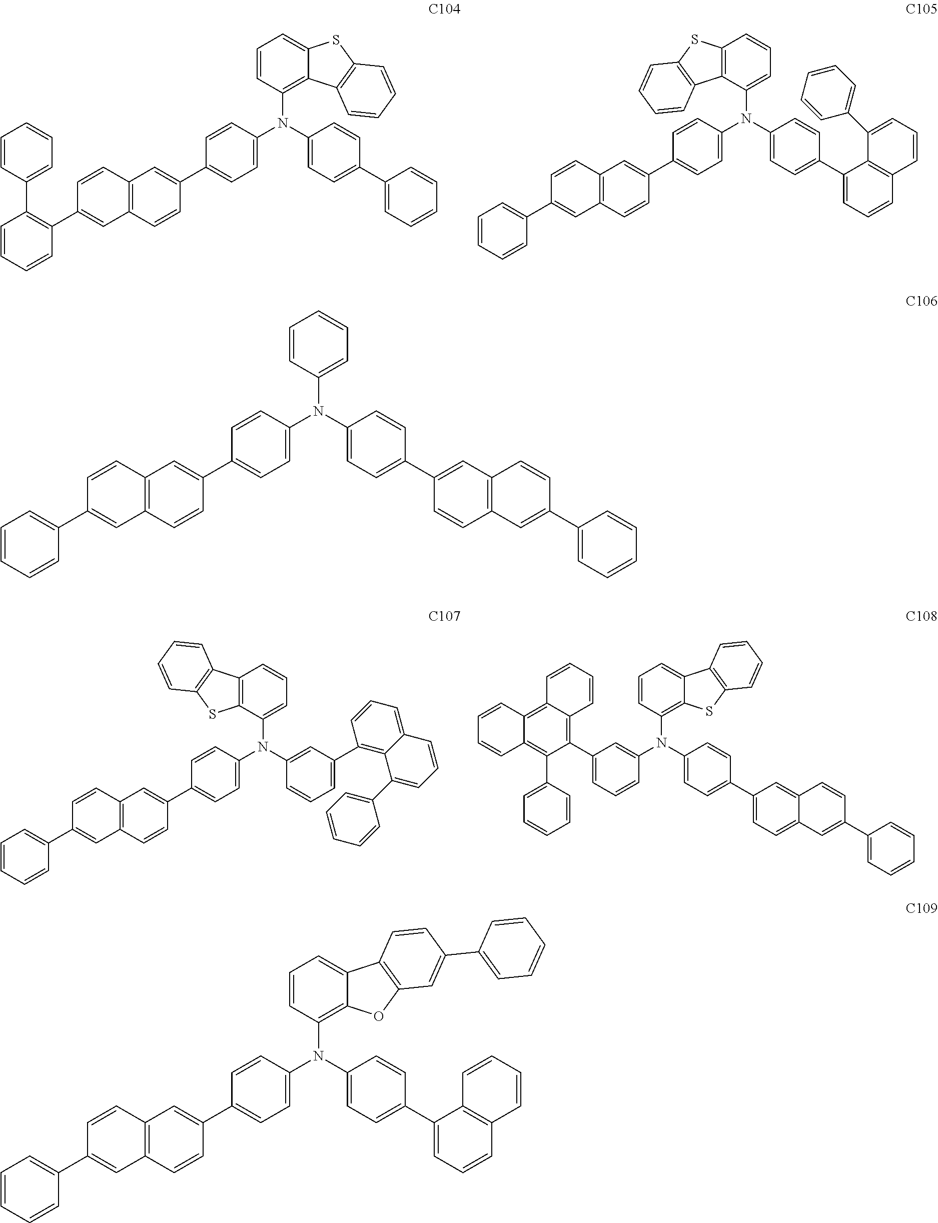

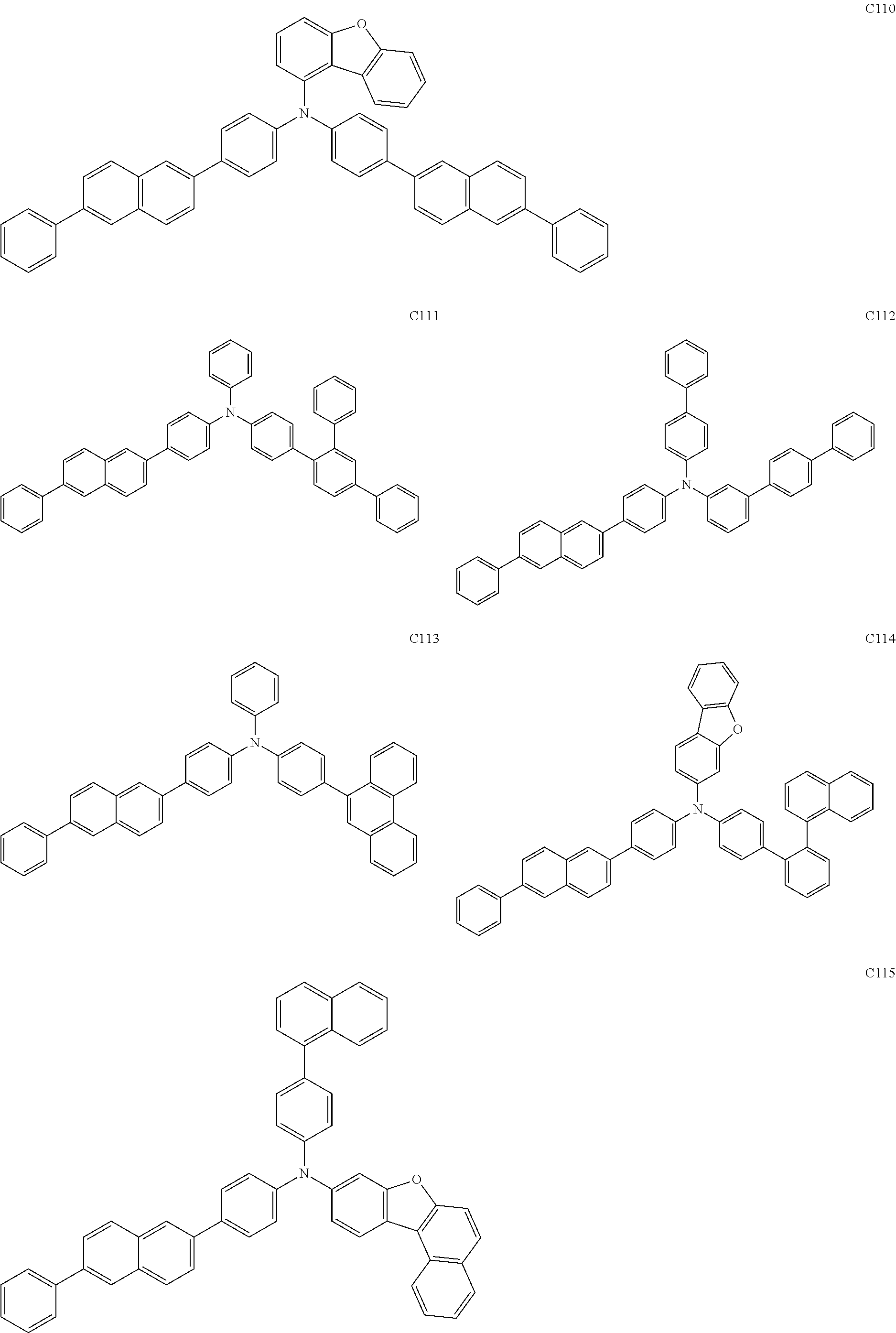

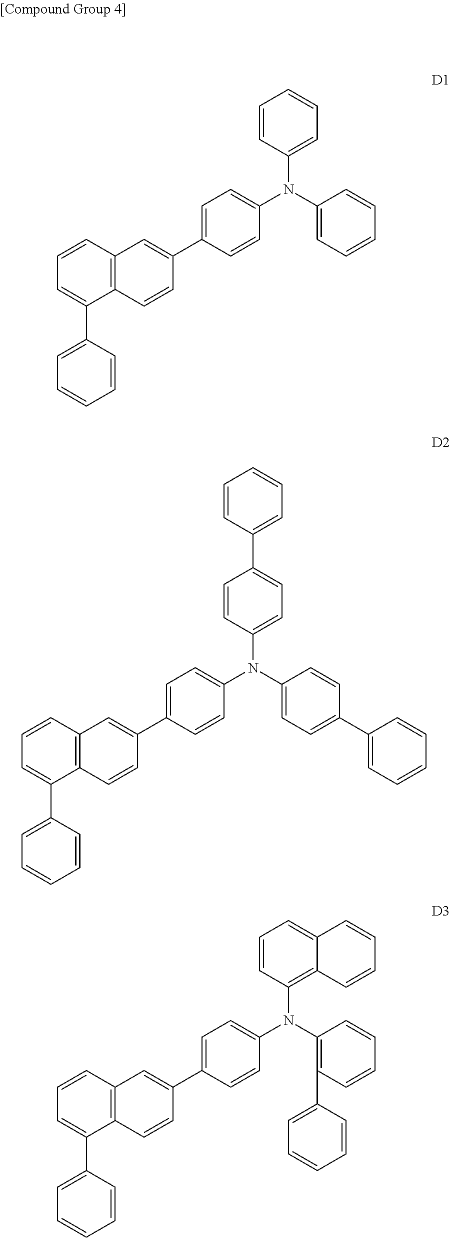 [SMILES: c1ccc(-c2cccc3cc(-c4ccc(N(c5ccccc5)c5ccccc5)cc4)ccc23)cc1 ; c1ccc(-c2ccc(N(c3ccc(-c4ccccc4)cc3)c3ccc(-c4ccc5c(-c6ccccc6)cccc5c4)cc3)cc2)cc1 ; c1ccc(-c2ccccc2N(c2ccc(-c3ccc4c(-c5ccccc5)cccc4c3)cc2)c2cccc3ccccc23)cc1]

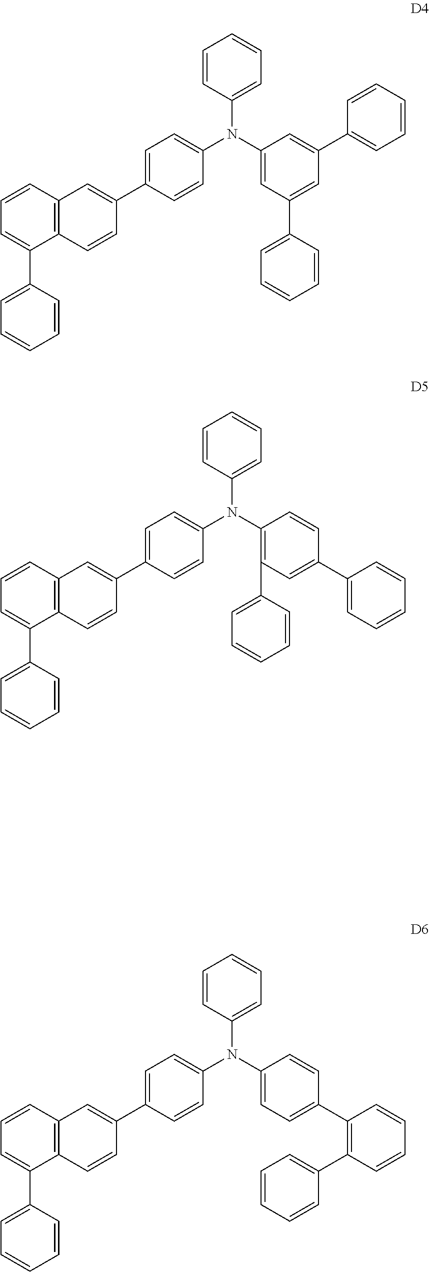 [SMILES: c1ccc(-c2cc(-c3ccccc3)cc(N(c3ccccc3)c3ccc(-c4ccc5c(-c6ccccc6)cccc5c4)cc3)c2)cc1 ; c1ccc(-c2ccc(N(c3ccccc3)c3ccc(-c4ccc5c(-c6ccccc6)cccc5c4)cc3)c(-c3ccccc3)c2)cc1 ; c1ccc(-c2ccccc2-c2ccc(N(c3ccccc3)c3ccc(-c4ccc5c(-c6ccccc6)cccc5c4)cc3)cc2)cc1]

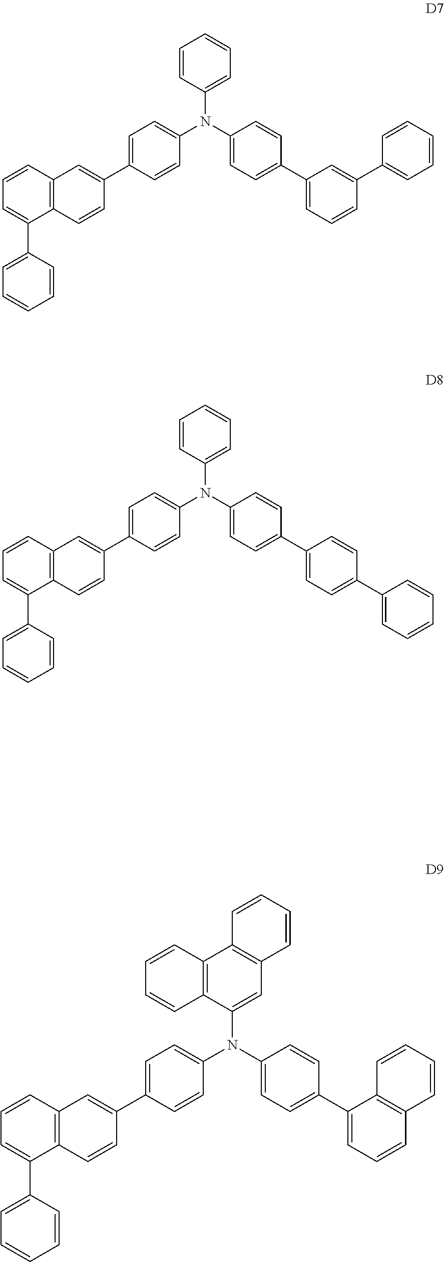 [SMILES: c1ccc(-c2cccc(-c3ccc(N(c4ccccc4)c4ccc(-c5ccc6c(-c7ccccc7)cccc6c5)cc4)cc3)c2)cc1 ; c1ccc(-c2ccc(-c3ccc(N(c4ccccc4)c4ccc(-c5ccc6c(-c7ccccc7)cccc6c5)cc4)cc3)cc2)cc1 ; c1ccc(-c2cccc3cc(-c4ccc(N(c5ccc(-c6cccc7ccccc67)cc5)c5cc6ccccc6c6ccccc56)cc4)ccc23)cc1]

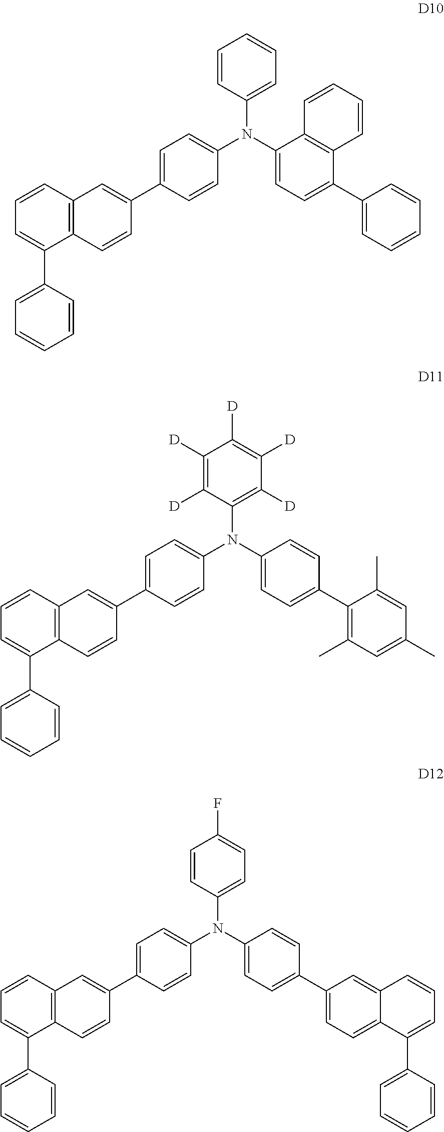 [SMILES: c1ccc(-c2cccc3cc(-c4ccc(N(c5ccccc5)c5ccc(-c6ccccc6)c6ccccc56)cc4)ccc23)cc1 ; [2H]c1c([2H])c([2H])c(N(c2ccc(-c3ccc4c(-c5ccccc5)cccc4c3)cc2)c2ccc(-c3c(C)cc(C)cc3C)cc2)c([2H])c1[2H] ; Fc1ccc(N(c2ccc(-c3ccc4c(-c5ccccc5)cccc4c3)cc2)c2ccc(-c3ccc4c(-c5ccccc5)cccc4c3)cc2)cc1]

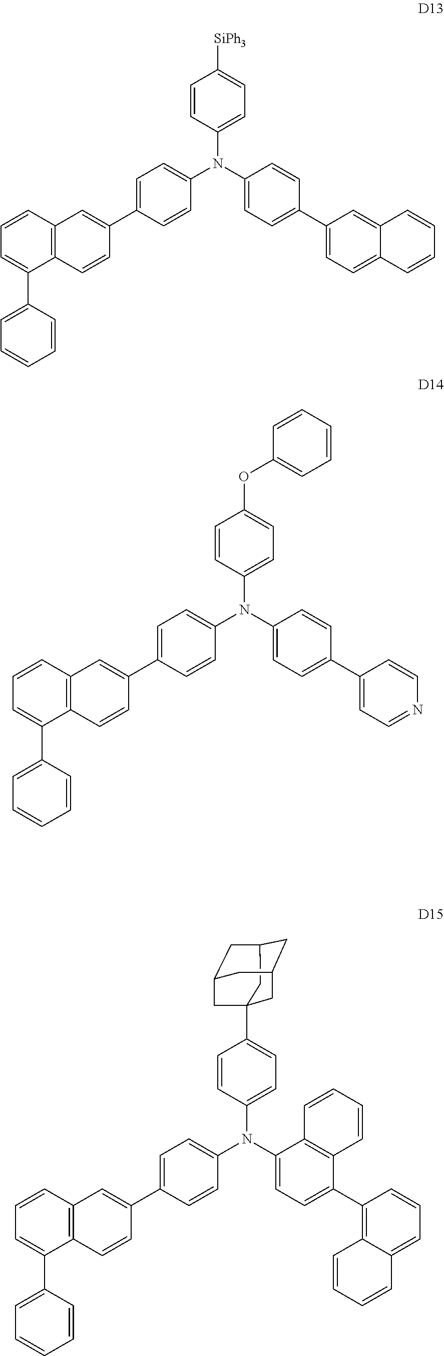

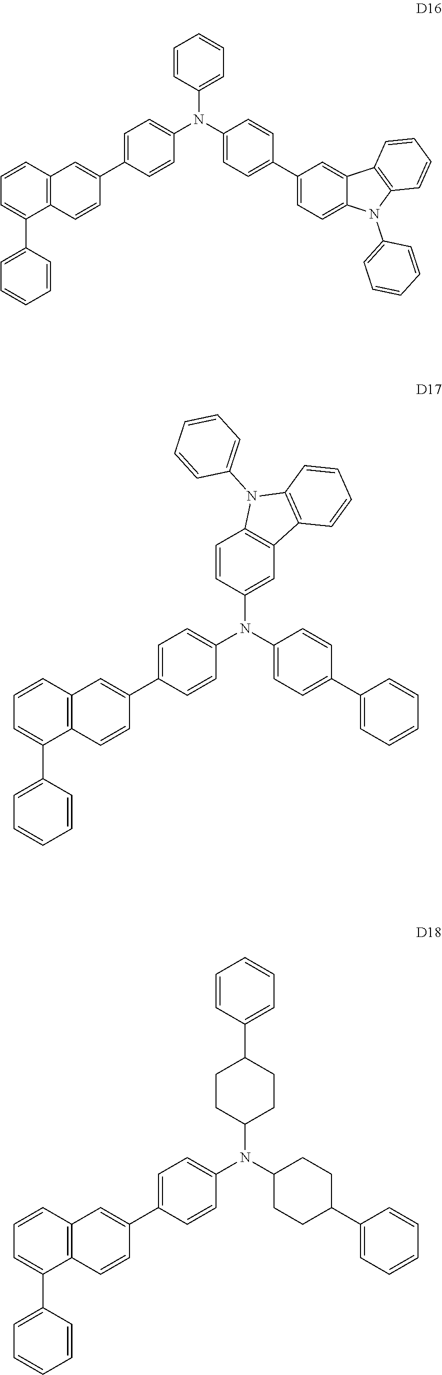 [SMILES: c1ccc(-c2cccc3cc(-c4ccc(N(c5ccccc5)c5ccc(-c6ccc7c(c6)c6ccccc6n7-c6ccccc6)cc5)cc4)ccc23)cc1 ; c1ccc(-c2ccc(N(c3ccc(-c4ccc5c(-c6ccccc6)cccc5c4)cc3)c3ccc4c(c3)c3ccccc3n4-c3ccccc3)cc2)cc1 ; c1ccc(-c2cccc3cc(-c4ccc(N(C5CCC(c6ccccc6)CC5)C5CCC(c6ccccc6)CC5)cc4)ccc23)cc1]

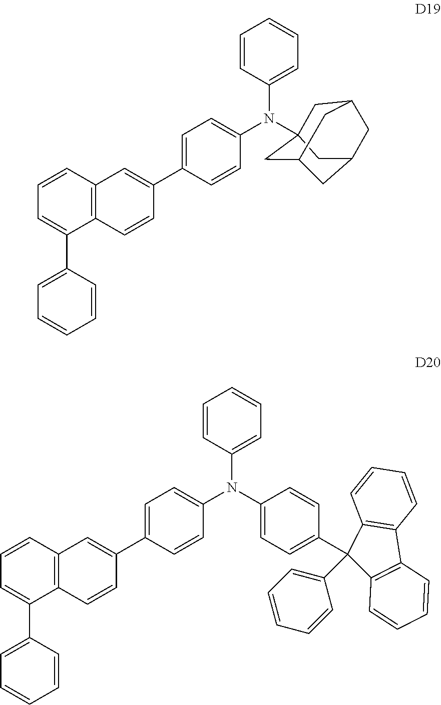 [SMILES: c1ccc(-c2cccc3cc(-c4ccc(N(c5ccccc5)C56CC7CC(CC(C7)C5)C6)cc4)ccc23)cc1 ; c1ccc(-c2cccc3cc(-c4ccc(N(c5ccccc5)c5ccc(C6(c7ccccc7)c7ccccc7-c7ccccc76)cc5)cc4)ccc23)cc1]

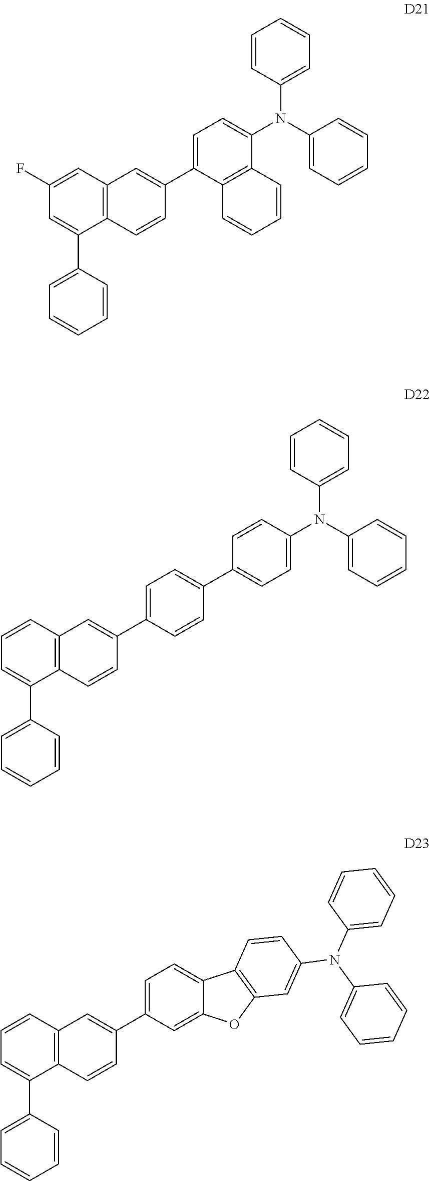 [SMILES: Fc1cc(-c2ccccc2)c2ccc(-c3ccc(N(c4ccccc4)c4ccccc4)c4ccccc34)cc2c1 ; c1ccc(-c2cccc3cc(-c4ccc(-c5ccc(N(c6ccccc6)c6ccccc6)cc5)cc4)ccc23)cc1 ; c1ccc(-c2cccc3cc(-c4ccc5c(c4)oc4cc(N(c6ccccc6)c6ccccc6)ccc45)ccc23)cc1]

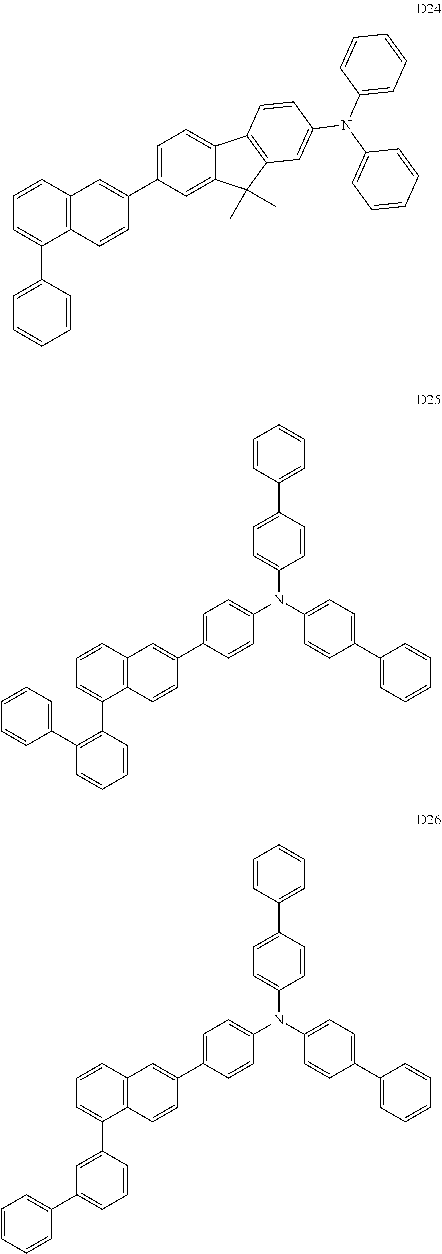 [SMILES: CC1(C)c2cc(-c3ccc4c(-c5ccccc5)cccc4c3)ccc2-c2ccc(N(c3ccccc3)c3ccccc3)cc21 ; c1ccc(-c2ccc(N(c3ccc(-c4ccccc4)cc3)c3ccc(-c4ccc5c(-c6ccccc6-c6ccccc6)cccc5c4)cc3)cc2)cc1 ; c1ccc(-c2ccc(N(c3ccc(-c4ccccc4)cc3)c3ccc(-c4ccc5c(-c6cccc(-c7ccccc7)c6)cccc5c4)cc3)cc2)cc1]

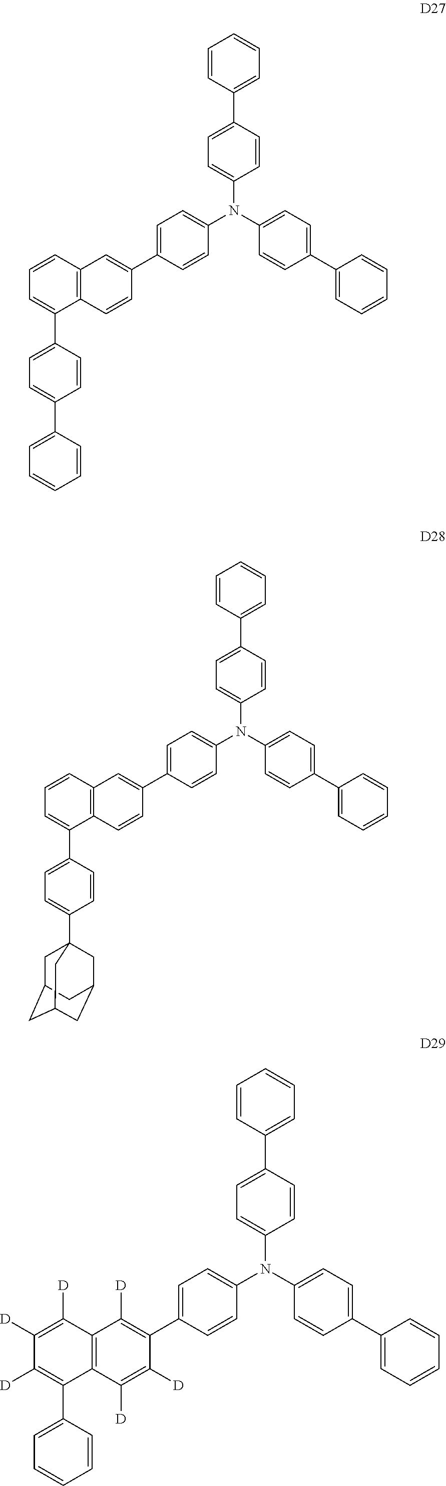 [SMILES: c1ccc(-c2ccc(-c3cccc4cc(-c5ccc(N(c6ccc(-c7ccccc7)cc6)c6ccc(-c7ccccc7)cc6)cc5)ccc34)cc2)cc1 ; c1ccc(-c2ccc(N(c3ccc(-c4ccccc4)cc3)c3ccc(-c4ccc5c(-c6ccc(C78CC9CC(CC(C9)C7)C8)cc6)cccc5c4)cc3)cc2)cc1 ; [2H]c1c([2H])c(-c2ccccc2)c2c([2H])c([2H])c(-c3ccc(N(c4ccc(-c5ccccc5)cc4)c4ccc(-c5ccccc5)cc4)cc3)c([2H])c2c1[2H]]

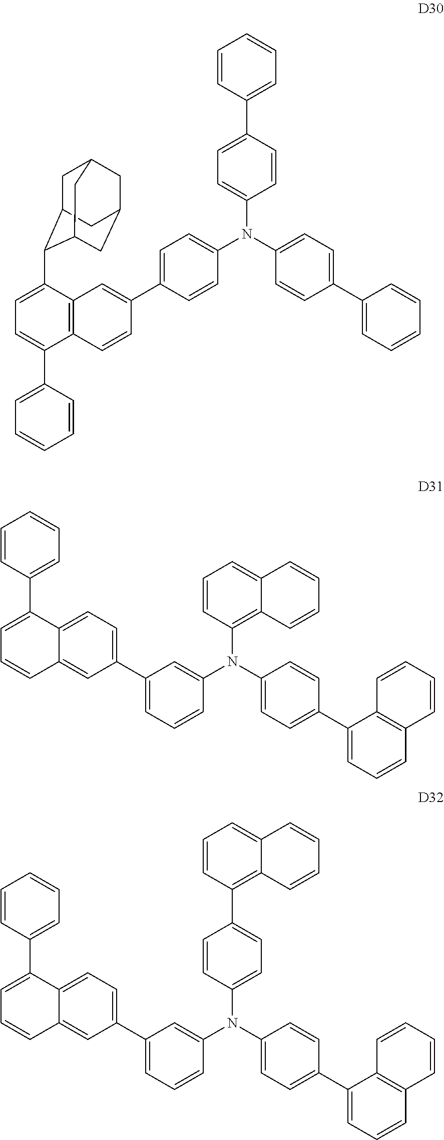 [SMILES: c1ccc(-c2ccc(N(c3ccc(-c4ccccc4)cc3)c3ccc(-c4ccc5c(-c6ccccc6)ccc(C6C7CC8CC(C7)CC6C8)c5c4)cc3)cc2)cc1 ; c1ccc(-c2cccc3cc(-c4cccc(N(c5ccc(-c6cccc7ccccc67)cc5)c5cccc6ccccc56)c4)ccc23)cc1 ; c1ccc(-c2cccc3cc(-c4cccc(N(c5ccc(-c6cccc7ccccc67)cc5)c5ccc(-c6cccc7ccccc67)cc5)c4)ccc23)cc1]

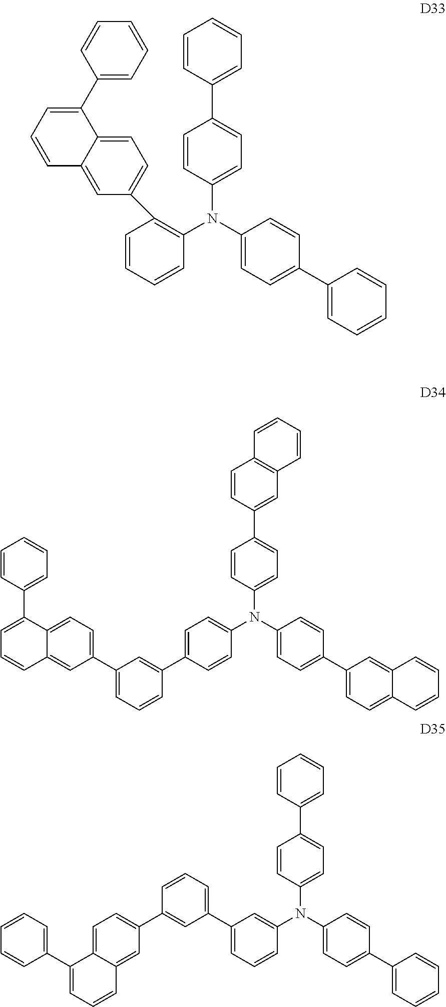 [SMILES: c1ccc(-c2ccc(N(c3ccc(-c4ccccc4)cc3)c3ccccc3-c3ccc4c(-c5ccccc5)cccc4c3)cc2)cc1 ; c1ccc(-c2cccc3cc(-c4cccc(-c5ccc(N(c6ccc(-c7ccc8ccccc8c7)cc6)c6ccc(-c7ccc8ccccc8c7)cc6)cc5)c4)ccc23)cc1 ; c1ccc(-c2ccc(N(c3ccc(-c4ccccc4)cc3)c3cccc(-c4cccc(-c5ccc6c(-c7ccccc7)cccc6c5)c4)c3)cc2)cc1]

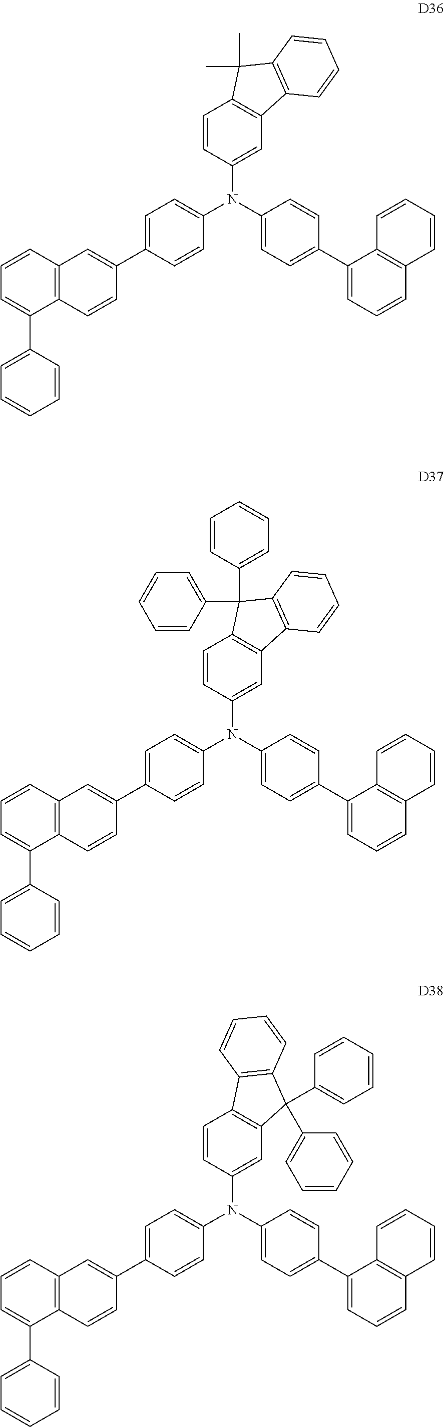 [SMILES: CC1(C)c2ccccc2-c2cc(N(c3ccc(-c4ccc5c(-c6ccccc6)cccc5c4)cc3)c3ccc(-c4cccc5ccccc45)cc3)ccc21 ; c1ccc(-c2cccc3cc(-c4ccc(N(c5ccc(-c6cccc7ccccc67)cc5)c5ccc6c(c5)-c5ccccc5C6(c5ccccc5)c5ccccc5)cc4)ccc23)cc1 ; c1ccc(-c2cccc3cc(-c4ccc(N(c5ccc(-c6cccc7ccccc67)cc5)c5ccc6c(c5)C(c5ccccc5)(c5ccccc5)c5ccccc5-6)cc4)ccc23)cc1]

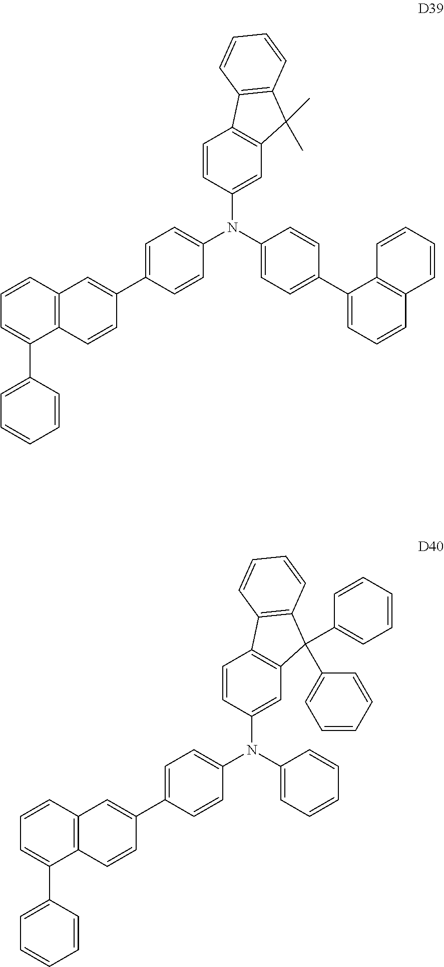 [SMILES: CC1(C)c2ccccc2-c2ccc(N(c3ccc(-c4ccc5c(-c6ccccc6)cccc5c4)cc3)c3ccc(-c4cccc5ccccc45)cc3)cc21 ; c1ccc(-c2cccc3cc(-c4ccc(N(c5ccccc5)c5ccc6c(c5)C(c5ccccc5)(c5ccccc5)c5ccccc5-6)cc4)ccc23)cc1]

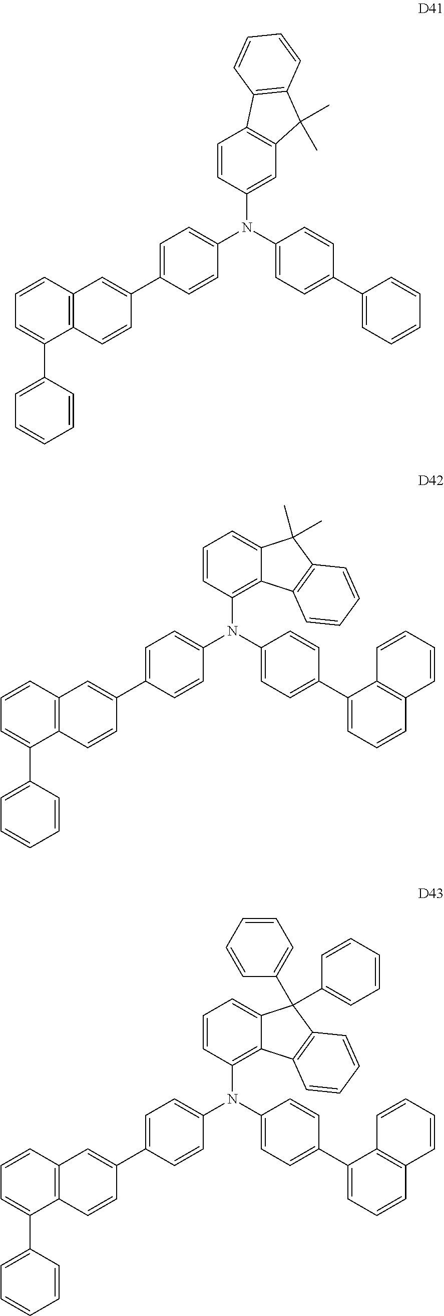 [SMILES: CC1(C)c2ccccc2-c2ccc(N(c3ccc(-c4ccccc4)cc3)c3ccc(-c4ccc5c(-c6ccccc6)cccc5c4)cc3)cc21 ; CC1(C)c2ccccc2-c2c(N(c3ccc(-c4ccc5c(-c6ccccc6)cccc5c4)cc3)c3ccc(-c4cccc5ccccc45)cc3)cccc21 ; c1ccc(-c2cccc3cc(-c4ccc(N(c5ccc(-c6cccc7ccccc67)cc5)c5cccc6c5-c5ccccc5C6(c5ccccc5)c5ccccc5)cc4)ccc23)cc1]

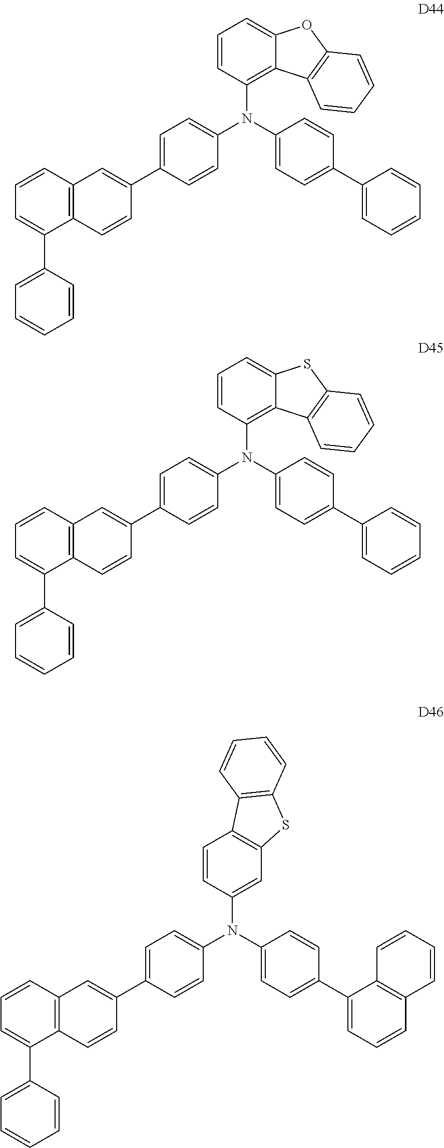 [SMILES: c1ccc(-c2ccc(N(c3ccc(-c4ccc5c(-c6ccccc6)cccc5c4)cc3)c3cccc4oc5ccccc5c34)cc2)cc1 ; c1ccc(-c2ccc(N(c3ccc(-c4ccc5c(-c6ccccc6)cccc5c4)cc3)c3cccc4sc5ccccc5c34)cc2)cc1 ; c1ccc(-c2cccc3cc(-c4ccc(N(c5ccc(-c6cccc7ccccc67)cc5)c5ccc6c(c5)sc5ccccc56)cc4)ccc23)cc1]

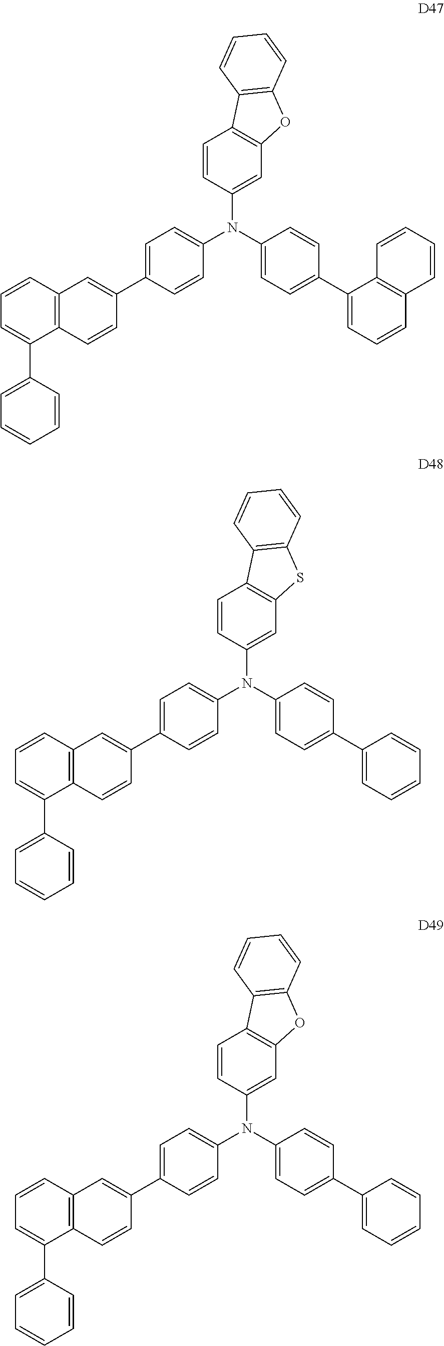 [SMILES: c1ccc(-c2cccc3cc(-c4ccc(N(c5ccc(-c6cccc7ccccc67)cc5)c5ccc6c(c5)oc5ccccc56)cc4)ccc23)cc1 ; c1ccc(-c2ccc(N(c3ccc(-c4ccc5c(-c6ccccc6)cccc5c4)cc3)c3ccc4c(c3)sc3ccccc34)cc2)cc1 ; c1ccc(-c2ccc(N(c3ccc(-c4ccc5c(-c6ccccc6)cccc5c4)cc3)c3ccc4c(c3)oc3ccccc34)cc2)cc1]

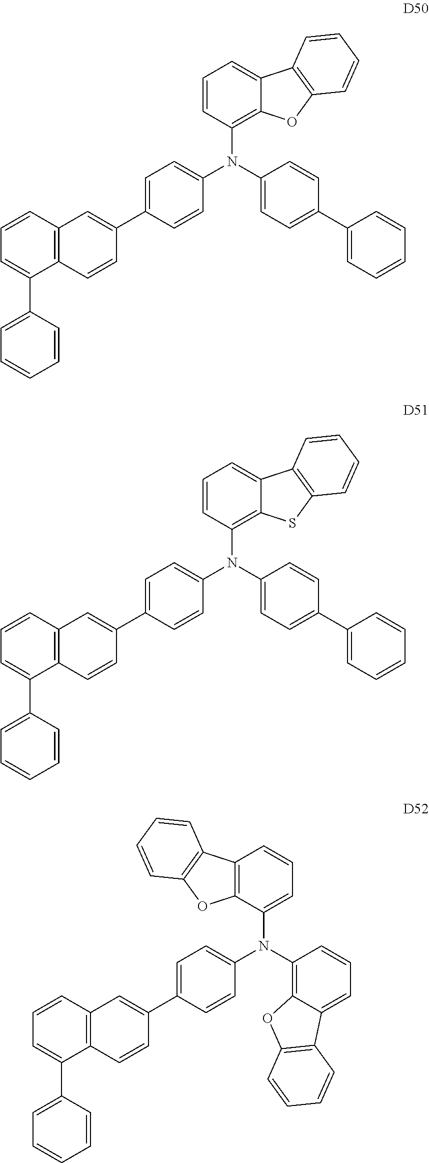 [SMILES: c1ccc(-c2ccc(N(c3ccc(-c4ccc5c(-c6ccccc6)cccc5c4)cc3)c3cccc4c3oc3ccccc34)cc2)cc1 ; c1ccc(-c2ccc(N(c3ccc(-c4ccc5c(-c6ccccc6)cccc5c4)cc3)c3cccc4c3sc3ccccc34)cc2)cc1 ; c1ccc(-c2cccc3cc(-c4ccc(N(c5cccc6c5oc5ccccc56)c5cccc6c5oc5ccccc56)cc4)ccc23)cc1]

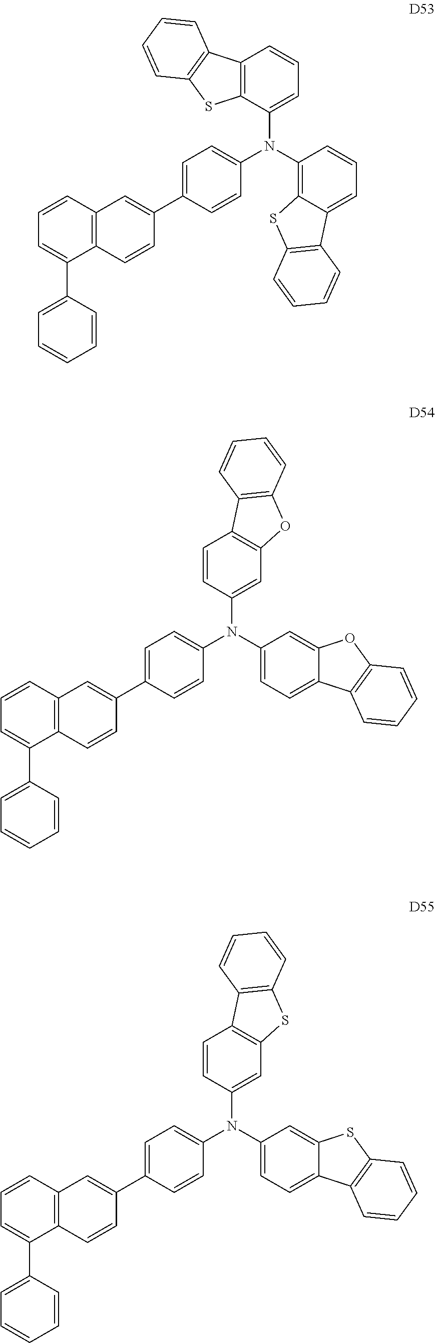 [SMILES: c1ccc(-c2cccc3cc(-c4ccc(N(c5cccc6c5sc5ccccc56)c5cccc6c5sc5ccccc56)cc4)ccc23)cc1 ; c1ccc(-c2cccc3cc(-c4ccc(N(c5ccc6c(c5)oc5ccccc56)c5ccc6c(c5)oc5ccccc56)cc4)ccc23)cc1 ; c1ccc(-c2cccc3cc(-c4ccc(N(c5ccc6c(c5)sc5ccccc56)c5ccc6c(c5)sc5ccccc56)cc4)ccc23)cc1]

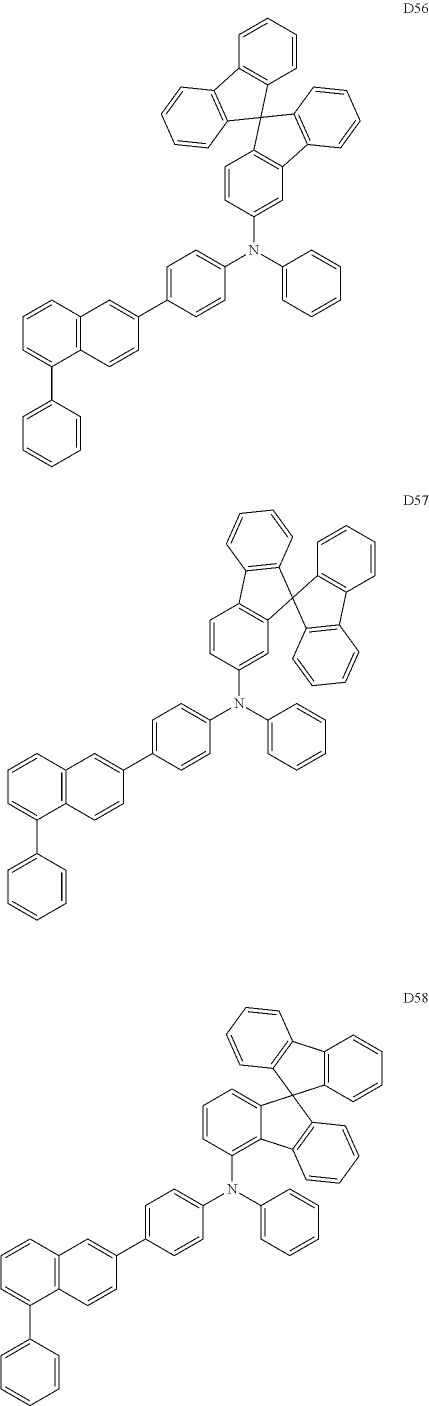 [SMILES: c1ccc(-c2cccc3cc(-c4ccc(N(c5ccccc5)c5ccc6c(c5)-c5ccccc5C65c6ccccc6-c6ccccc65)cc4)ccc23)cc1 ; c1ccc(-c2cccc3cc(-c4ccc(N(c5ccccc5)c5ccc6c(c5)C5(c7ccccc7-c7ccccc75)c5ccccc5-6)cc4)ccc23)cc1 ; c1ccc(-c2cccc3cc(-c4ccc(N(c5ccccc5)c5cccc6c5-c5ccccc5C65c6ccccc6-c6ccccc65)cc4)ccc23)cc1]

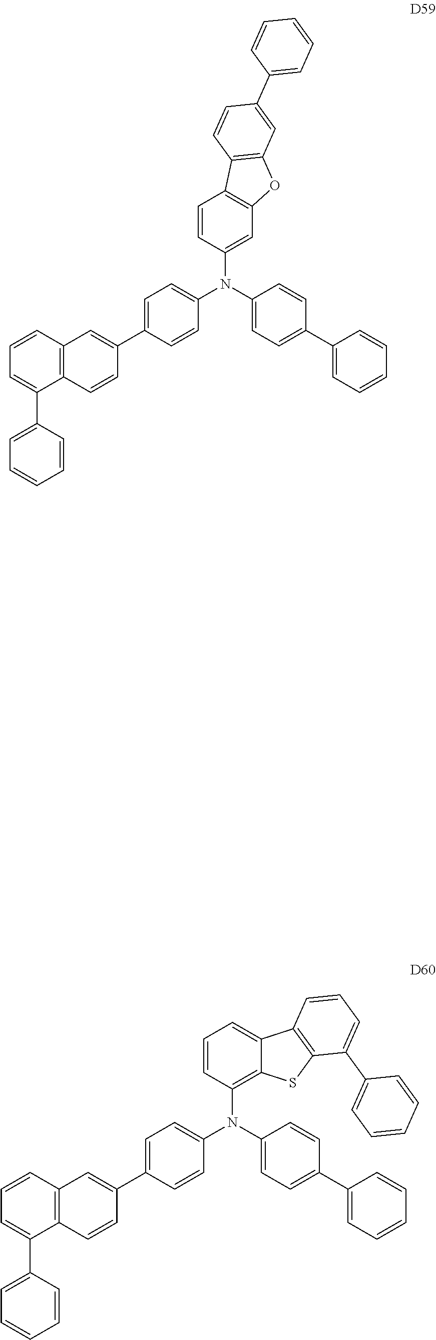 [SMILES: c1ccc(-c2ccc(N(c3ccc(-c4ccc5c(-c6ccccc6)cccc5c4)cc3)c3ccc4c(c3)oc3cc(-c5ccccc5)ccc34)cc2)cc1 ; c1ccc(-c2ccc(N(c3ccc(-c4ccc5c(-c6ccccc6)cccc5c4)cc3)c3cccc4c3sc3c(-c5ccccc5)cccc34)cc2)cc1]

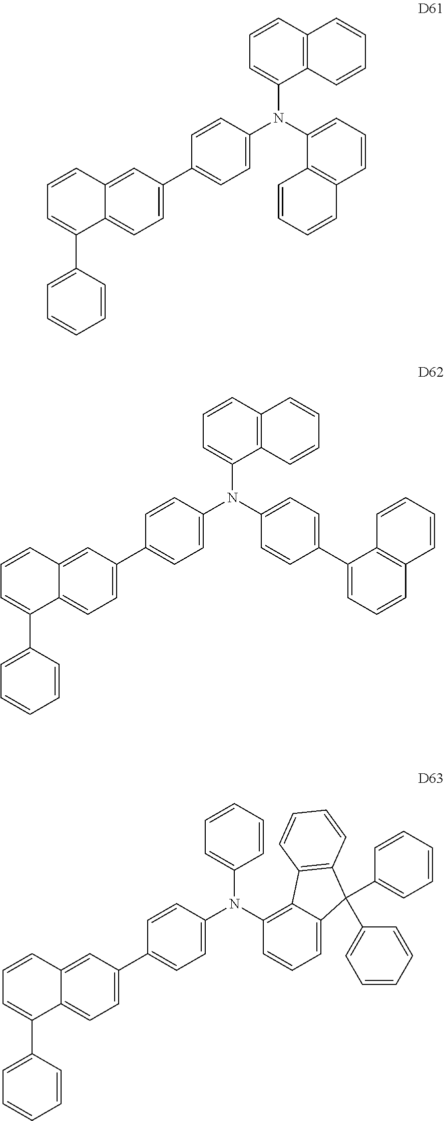 [SMILES: c1ccc(-c2cccc3cc(-c4ccc(N(c5cccc6ccccc56)c5cccc6ccccc56)cc4)ccc23)cc1 ; c1ccc(-c2cccc3cc(-c4ccc(N(c5ccc(-c6cccc7ccccc67)cc5)c5cccc6ccccc56)cc4)ccc23)cc1 ; c1ccc(-c2cccc3cc(-c4ccc(N(c5ccccc5)c5cccc6c5-c5ccccc5C6(c5ccccc5)c5ccccc5)cc4)ccc23)cc1]

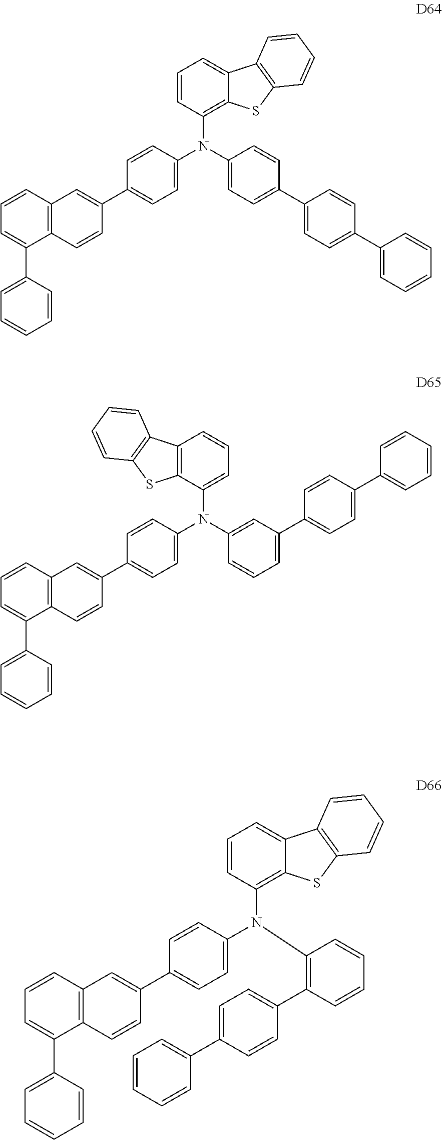 [SMILES: c1ccc(-c2ccc(-c3ccc(N(c4ccc(-c5ccc6c(-c7ccccc7)cccc6c5)cc4)c4cccc5c4sc4ccccc45)cc3)cc2)cc1 ; c1ccc(-c2ccc(-c3cccc(N(c4ccc(-c5ccc6c(-c7ccccc7)cccc6c5)cc4)c4cccc5c4sc4ccccc45)c3)cc2)cc1 ; c1ccc(-c2ccc(-c3ccccc3N(c3ccc(-c4ccc5c(-c6ccccc6)cccc5c4)cc3)c3cccc4c3sc3ccccc34)cc2)cc1]

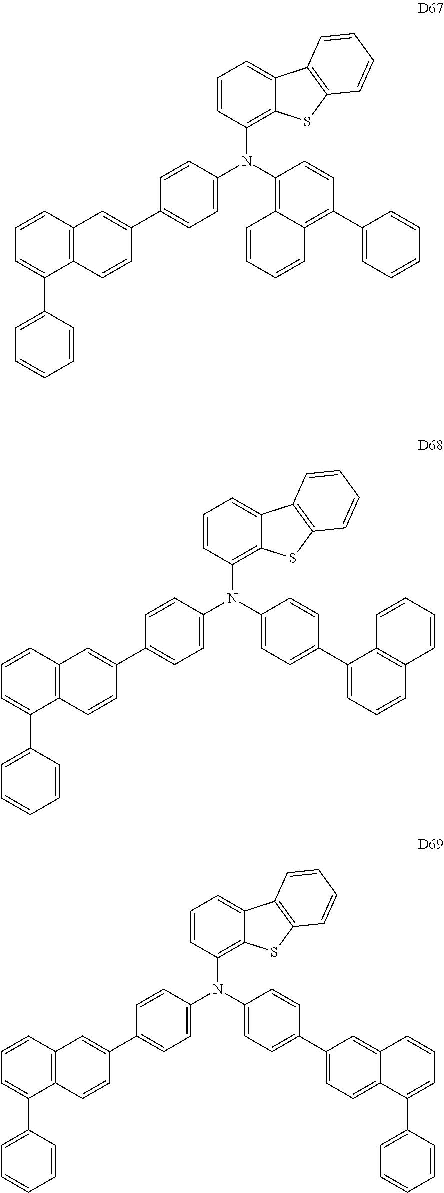 [SMILES: c1ccc(-c2cccc3cc(-c4ccc(N(c5ccc(-c6ccccc6)c6ccccc56)c5cccc6c5sc5ccccc56)cc4)ccc23)cc1 ; c1ccc(-c2cccc3cc(-c4ccc(N(c5ccc(-c6cccc7ccccc67)cc5)c5cccc6c5sc5ccccc56)cc4)ccc23)cc1 ; c1ccc(-c2cccc3cc(-c4ccc(N(c5ccc(-c6ccc7c(-c8ccccc8)cccc7c6)cc5)c5cccc6c5sc5ccccc56)cc4)ccc23)cc1]

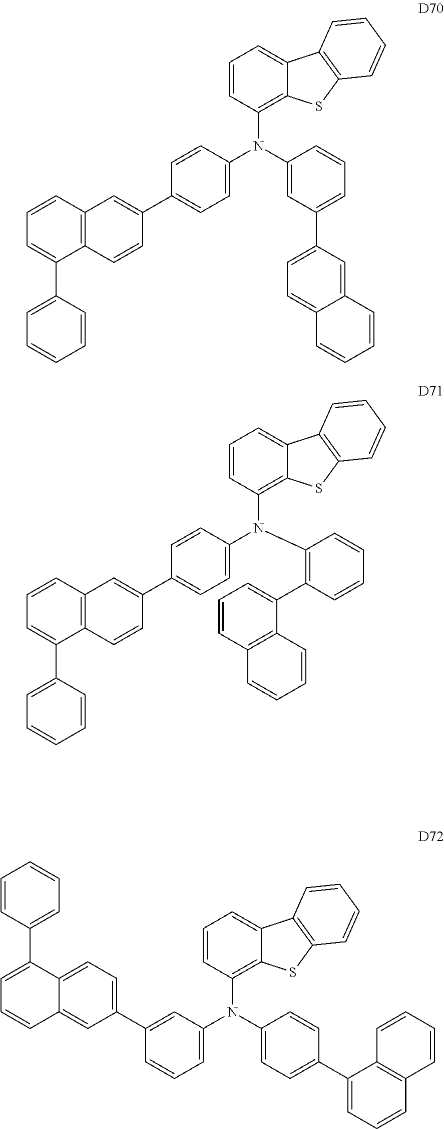

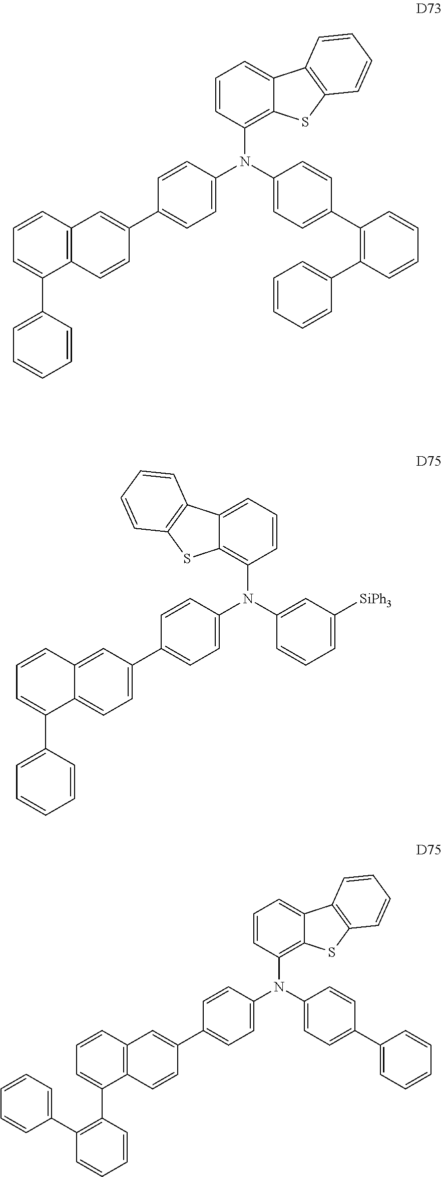

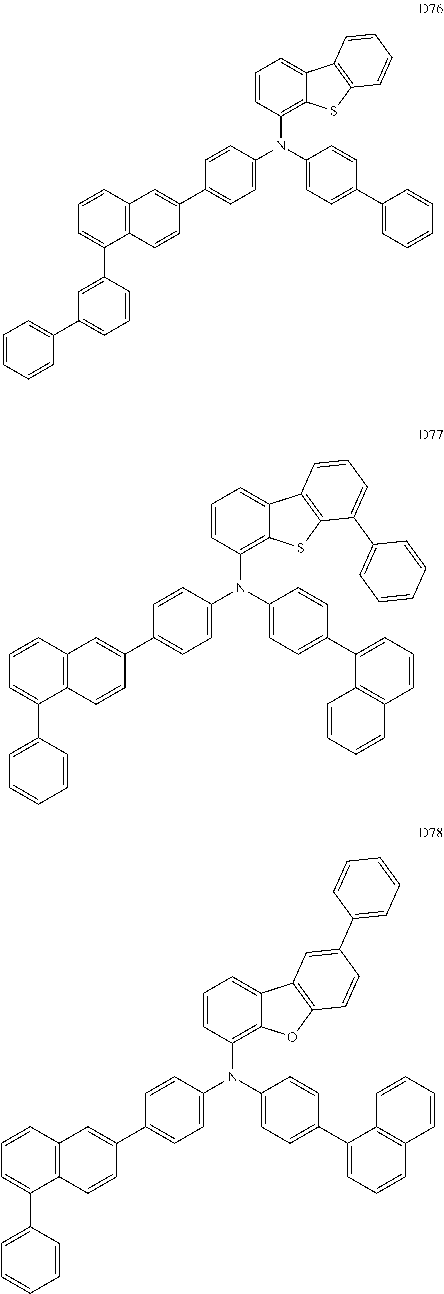 [SMILES: c1ccc(-c2ccc(N(c3ccc(-c4ccc5c(-c6cccc(-c7ccccc7)c6)cccc5c4)cc3)c3cccc4c3sc3ccccc34)cc2)cc1 ; c1ccc(-c2cccc3cc(-c4ccc(N(c5ccc(-c6cccc7ccccc67)cc5)c5cccc6c5sc5c(-c7ccccc7)cccc56)cc4)ccc23)cc1 ; c1ccc(-c2ccc3oc4c(N(c5ccc(-c6ccc7c(-c8ccccc8)cccc7c6)cc5)c5ccc(-c6cccc7ccccc67)cc5)cccc4c3c2)cc1]

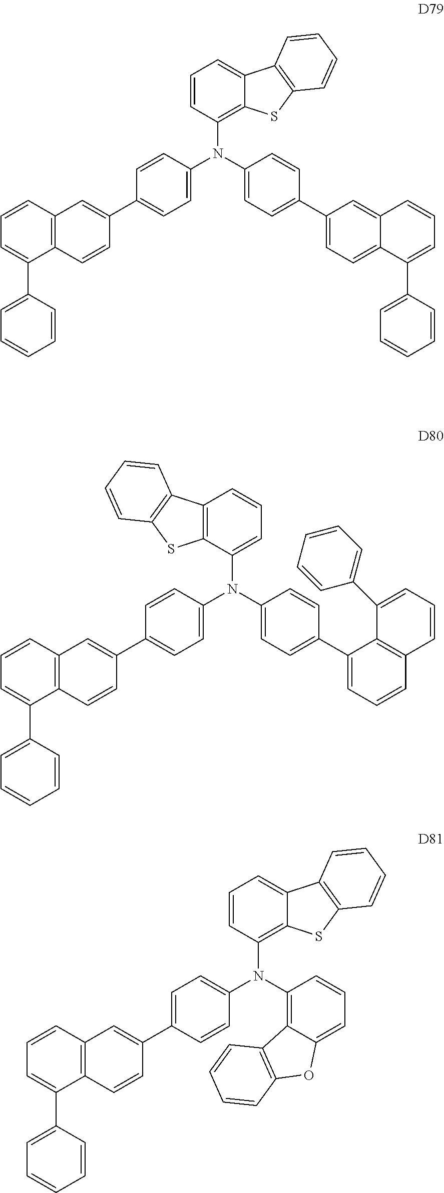 [SMILES: c1ccc(-c2cccc3cc(-c4ccc(N(c5ccc(-c6ccc7c(-c8ccccc8)cccc7c6)cc5)c5cccc6c5sc5ccccc56)cc4)ccc23)cc1 ; c1ccc(-c2cccc3cc(-c4ccc(N(c5ccc(-c6cccc7cccc(-c8ccccc8)c67)cc5)c5cccc6c5sc5ccccc56)cc4)ccc23)cc1 ; c1ccc(-c2cccc3cc(-c4ccc(N(c5cccc6c5sc5ccccc56)c5cccc6oc7ccccc7c56)cc4)ccc23)cc1]

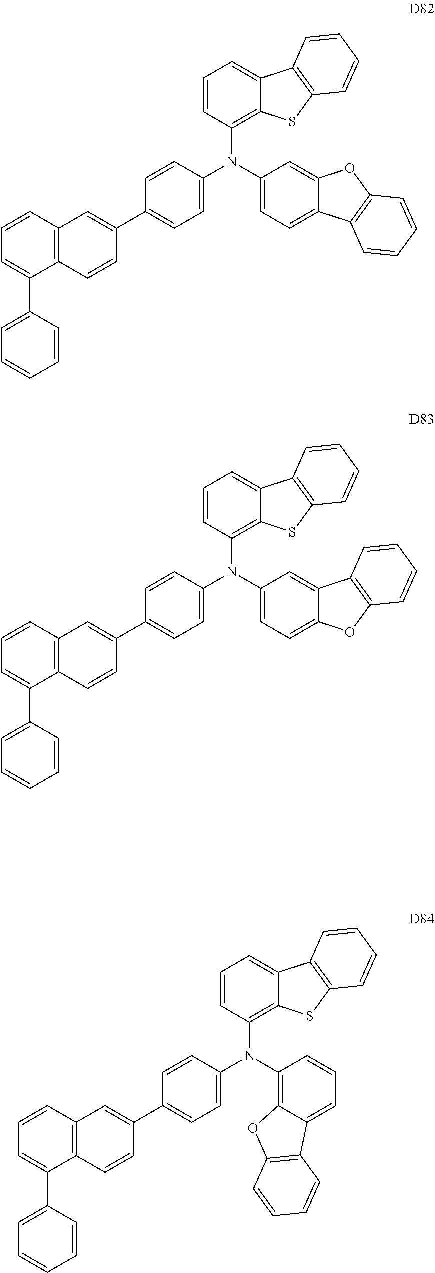 [SMILES: c1ccc(-c2cccc3cc(-c4ccc(N(c5ccc6c(c5)oc5ccccc56)c5cccc6c5sc5ccccc56)cc4)ccc23)cc1 ; c1ccc(-c2cccc3cc(-c4ccc(N(c5ccc6oc7ccccc7c6c5)c5cccc6c5sc5ccccc56)cc4)ccc23)cc1 ; c1ccc(-c2cccc3cc(-c4ccc(N(c5cccc6c5oc5ccccc56)c5cccc6c5sc5ccccc56)cc4)ccc23)cc1]

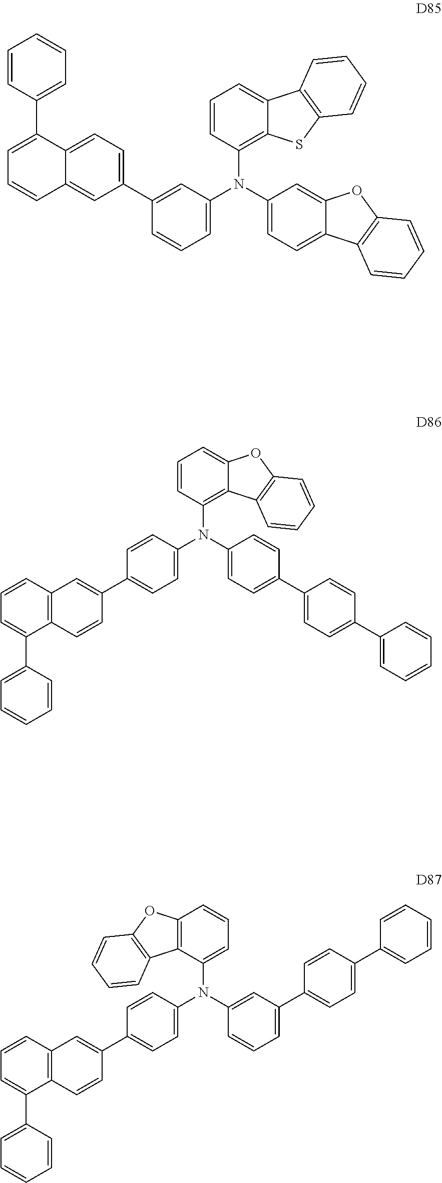 [SMILES: c1ccc(-c2cccc3cc(-c4cccc(N(c5ccc6c(c5)oc5ccccc56)c5cccc6c5sc5ccccc56)c4)ccc23)cc1 ; c1ccc(-c2ccc(-c3ccc(N(c4ccc(-c5ccc6c(-c7ccccc7)cccc6c5)cc4)c4cccc5oc6ccccc6c45)cc3)cc2)cc1 ; c1ccc(-c2ccc(-c3cccc(N(c4ccc(-c5ccc6c(-c7ccccc7)cccc6c5)cc4)c4cccc5oc6ccccc6c45)c3)cc2)cc1]

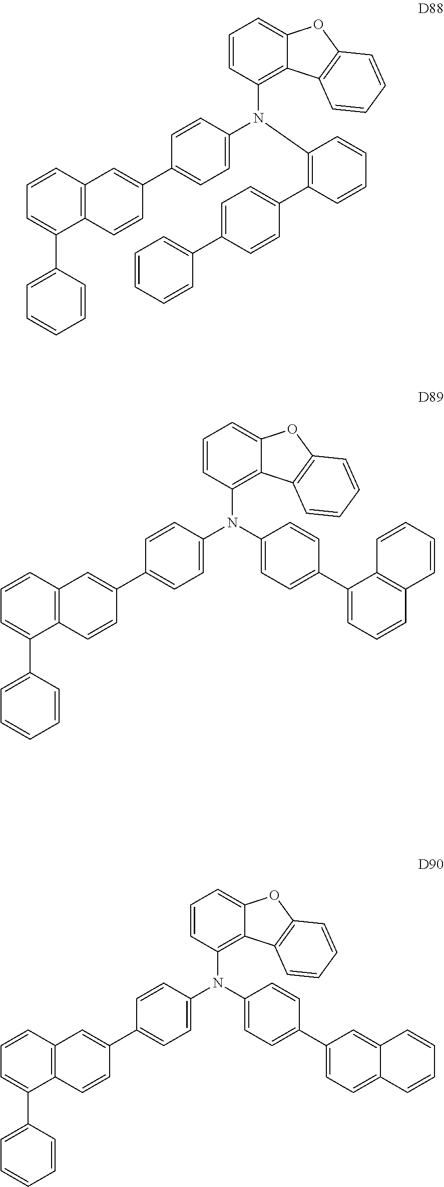 [SMILES: c1ccc(-c2ccc(-c3ccccc3N(c3ccc(-c4ccc5c(-c6ccccc6)cccc5c4)cc3)c3cccc4oc5ccccc5c34)cc2)cc1 ; c1ccc(-c2cccc3cc(-c4ccc(N(c5ccc(-c6cccc7ccccc67)cc5)c5cccc6oc7ccccc7c56)cc4)ccc23)cc1 ; c1ccc(-c2cccc3cc(-c4ccc(N(c5ccc(-c6ccc7ccccc7c6)cc5)c5cccc6oc7ccccc7c56)cc4)ccc23)cc1]

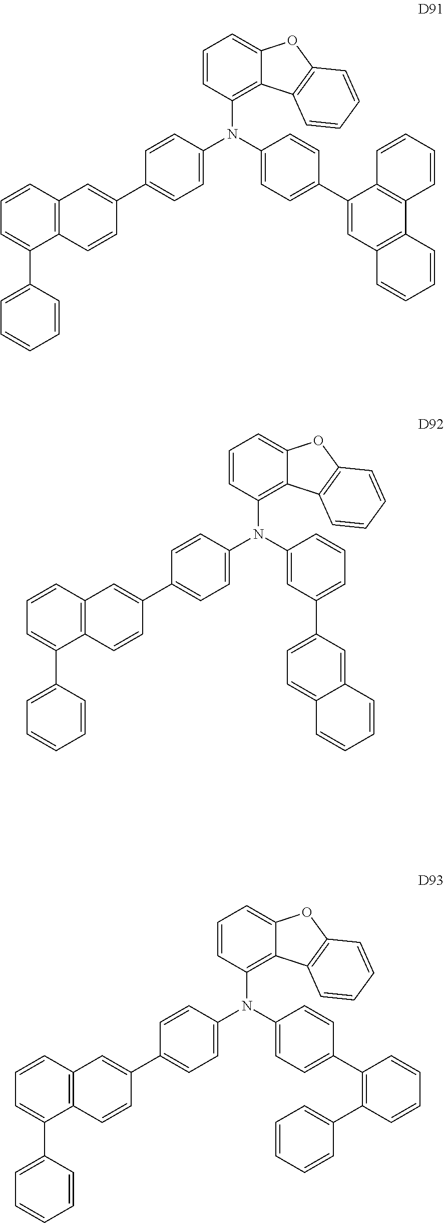 [SMILES: c1ccc(-c2cccc3cc(-c4ccc(N(c5ccc(-c6cc7ccccc7c7ccccc67)cc5)c5cccc6oc7ccccc7c56)cc4)ccc23)cc1 ; c1ccc(-c2cccc3cc(-c4ccc(N(c5cccc(-c6ccc7ccccc7c6)c5)c5cccc6oc7ccccc7c56)cc4)ccc23)cc1 ; c1ccc(-c2ccccc2-c2ccc(N(c3ccc(-c4ccc5c(-c6ccccc6)cccc5c4)cc3)c3cccc4oc5ccccc5c34)cc2)cc1]

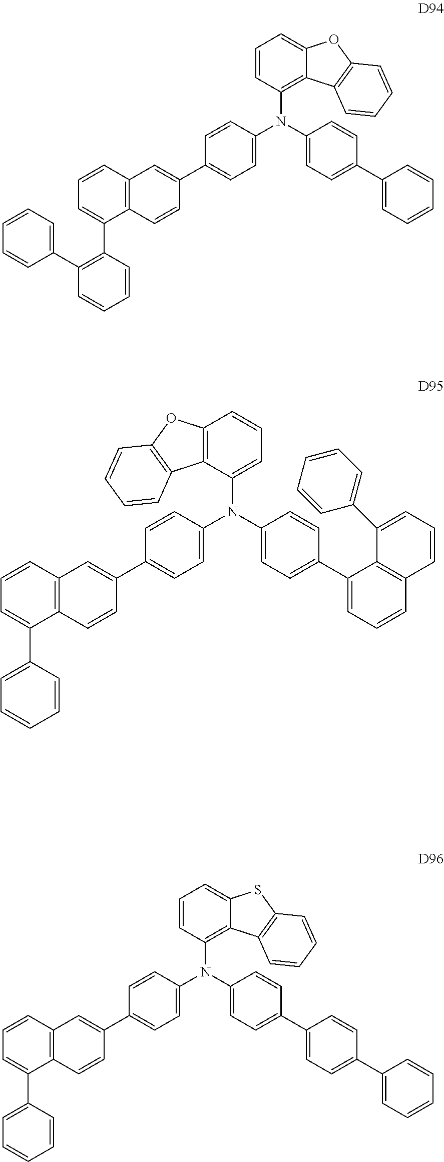 [SMILES: c1ccc(-c2ccc(N(c3ccc(-c4ccc5c(-c6ccccc6-c6ccccc6)cccc5c4)cc3)c3cccc4oc5ccccc5c34)cc2)cc1 ; c1ccc(-c2cccc3cc(-c4ccc(N(c5ccc(-c6cccc7cccc(-c8ccccc8)c67)cc5)c5cccc6oc7ccccc7c56)cc4)ccc23)cc1 ; c1ccc(-c2ccc(-c3ccc(N(c4ccc(-c5ccc6c(-c7ccccc7)cccc6c5)cc4)c4cccc5sc6ccccc6c45)cc3)cc2)cc1]

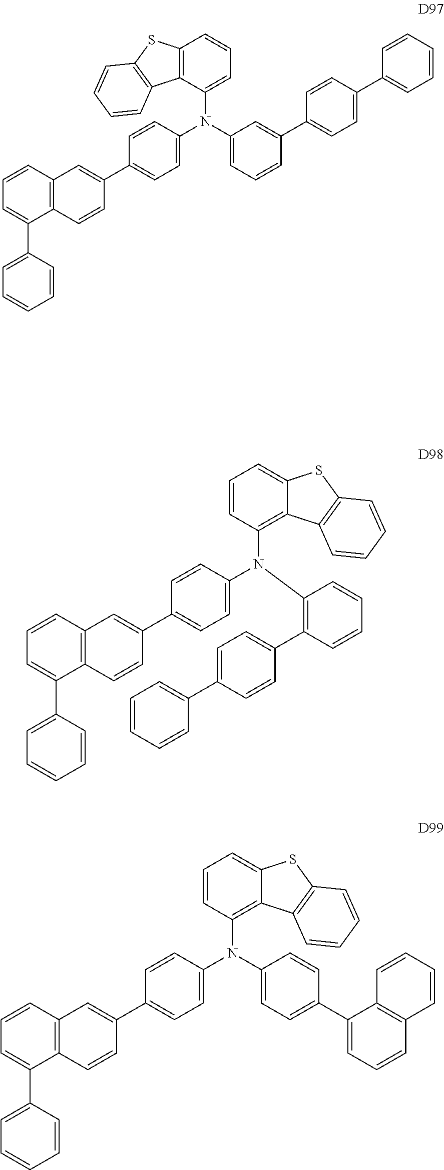 [SMILES: c1ccc(-c2ccc(-c3cccc(N(c4ccc(-c5ccc6c(-c7ccccc7)cccc6c5)cc4)c4cccc5sc6ccccc6c45)c3)cc2)cc1 ; c1ccc(-c2ccc(-c3ccccc3N(c3ccc(-c4ccc5c(-c6ccccc6)cccc5c4)cc3)c3cccc4sc5ccccc5c34)cc2)cc1 ; c1ccc(-c2cccc3cc(-c4ccc(N(c5ccc(-c6cccc7ccccc67)cc5)c5cccc6sc7ccccc7c56)cc4)ccc23)cc1]

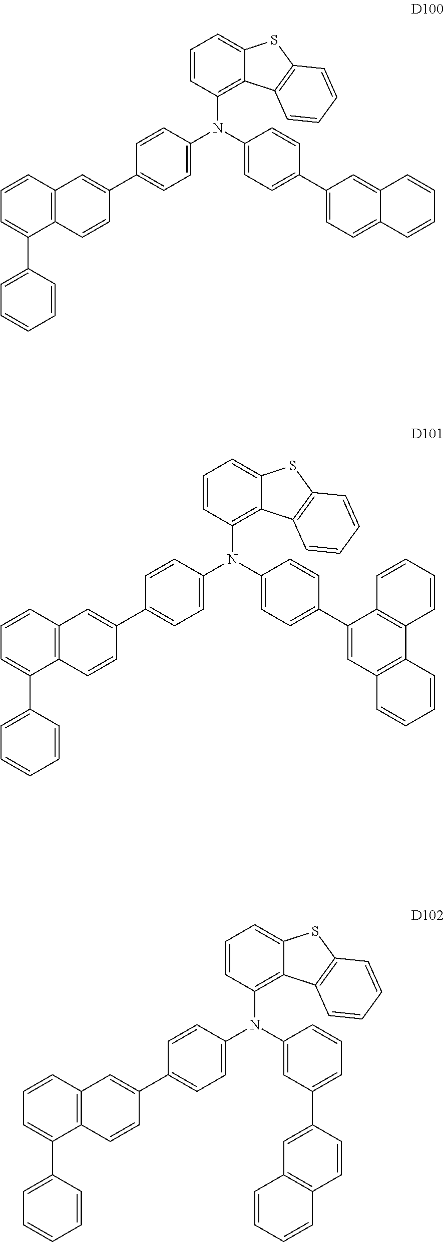 [SMILES: c1ccc(-c2cccc3cc(-c4ccc(N(c5ccc(-c6ccc7ccccc7c6)cc5)c5cccc6sc7ccccc7c56)cc4)ccc23)cc1 ; c1ccc(-c2cccc3cc(-c4ccc(N(c5ccc(-c6cc7ccccc7c7ccccc67)cc5)c5cccc6sc7ccccc7c56)cc4)ccc23)cc1 ; c1ccc(-c2cccc3cc(-c4ccc(N(c5cccc(-c6ccc7ccccc7c6)c5)c5cccc6sc7ccccc7c56)cc4)ccc23)cc1]

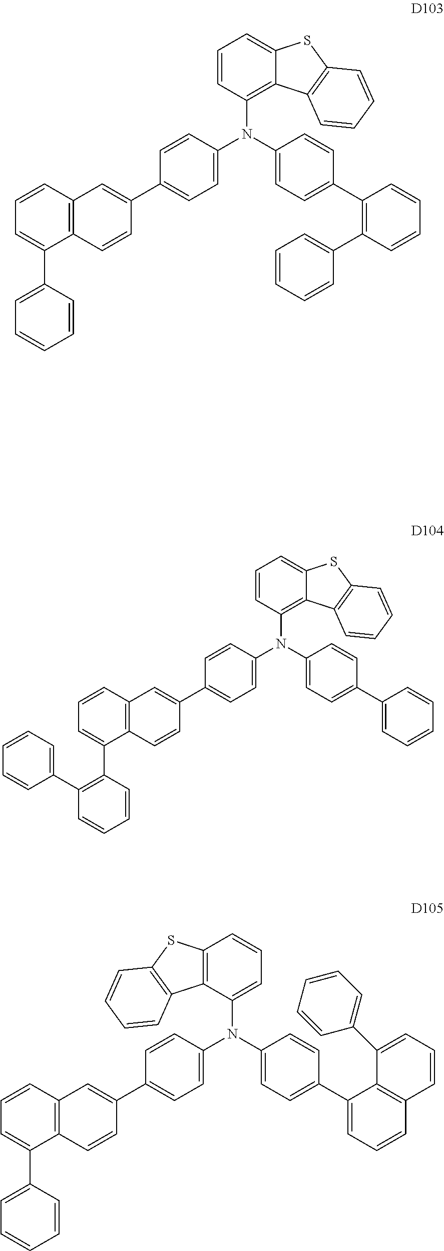 [SMILES: c1ccc(-c2ccccc2-c2ccc(N(c3ccc(-c4ccc5c(-c6ccccc6)cccc5c4)cc3)c3cccc4sc5ccccc5c34)cc2)cc1 ; c1ccc(-c2ccc(N(c3ccc(-c4ccc5c(-c6ccccc6-c6ccccc6)cccc5c4)cc3)c3cccc4sc5ccccc5c34)cc2)cc1 ; c1ccc(-c2cccc3cc(-c4ccc(N(c5ccc(-c6cccc7cccc(-c8ccccc8)c67)cc5)c5cccc6sc7ccccc7c56)cc4)ccc23)cc1]

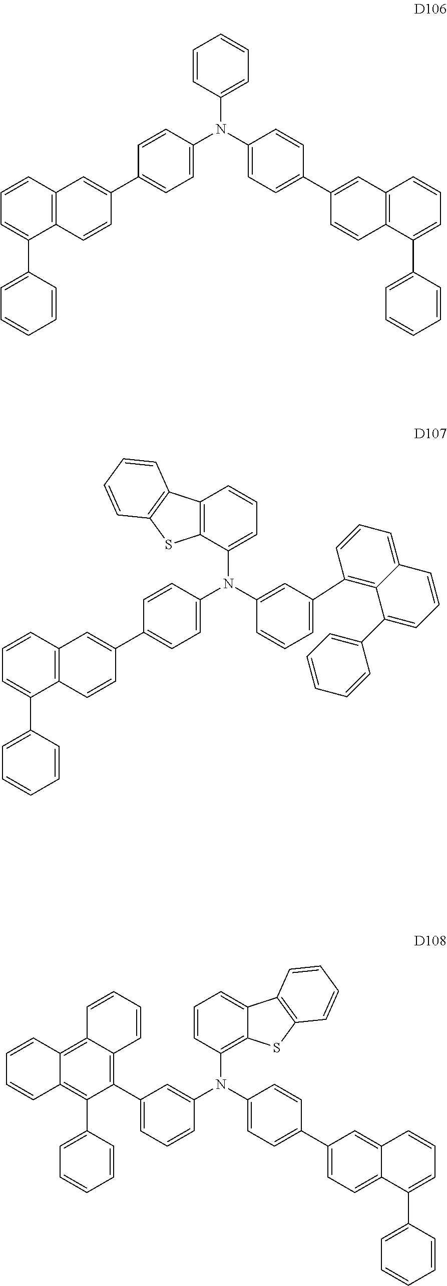 [SMILES: c1ccc(-c2cccc3cc(-c4ccc(N(c5ccccc5)c5ccc(-c6ccc7c(-c8ccccc8)cccc7c6)cc5)cc4)ccc23)cc1 ; c1ccc(-c2cccc3cc(-c4ccc(N(c5cccc(-c6cccc7cccc(-c8ccccc8)c67)c5)c5cccc6c5sc5ccccc56)cc4)ccc23)cc1 ; c1ccc(-c2cccc3cc(-c4ccc(N(c5cccc(-c6c(-c7ccccc7)c7ccccc7c7ccccc67)c5)c5cccc6c5sc5ccccc56)cc4)ccc23)cc1]

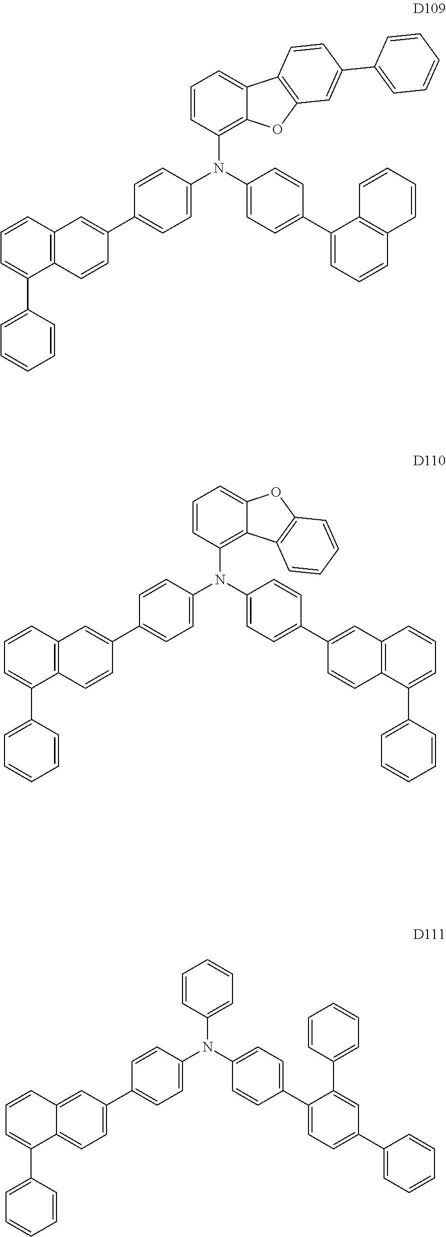 [SMILES: c1ccc(-c2ccc3c(c2)oc2c(N(c4ccc(-c5ccc6c(-c7ccccc7)cccc6c5)cc4)c4ccc(-c5cccc6ccccc56)cc4)cccc23)cc1 ; c1ccc(-c2cccc3cc(-c4ccc(N(c5ccc(-c6ccc7c(-c8ccccc8)cccc7c6)cc5)c5cccc6oc7ccccc7c56)cc4)ccc23)cc1 ; c1ccc(-c2ccc(-c3ccc(N(c4ccccc4)c4ccc(-c5ccc6c(-c7ccccc7)cccc6c5)cc4)cc3)c(-c3ccccc3)c2)cc1]

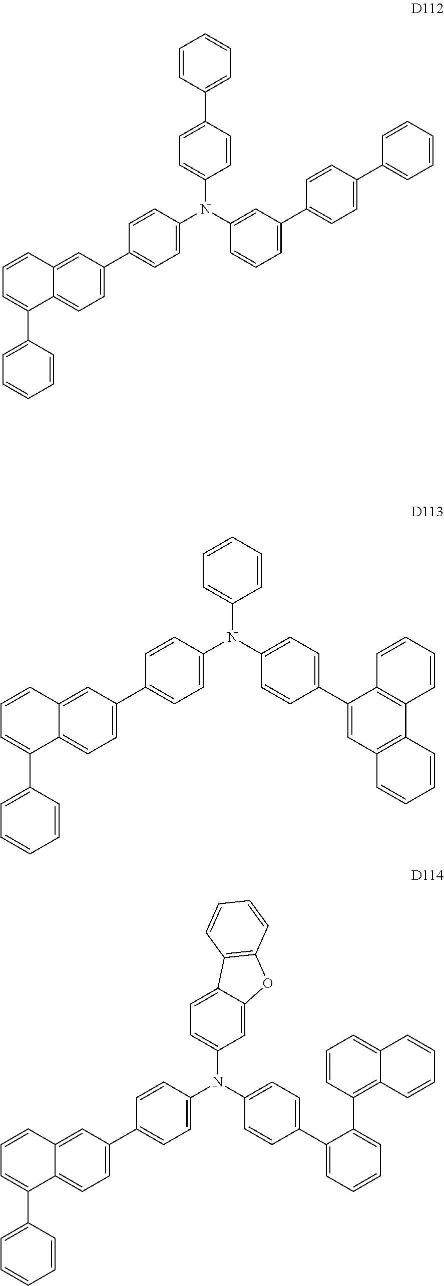 [SMILES: c1ccc(-c2ccc(-c3cccc(N(c4ccc(-c5ccccc5)cc4)c4ccc(-c5ccc6c(-c7ccccc7)cccc6c5)cc4)c3)cc2)cc1 ; c1ccc(-c2cccc3cc(-c4ccc(N(c5ccccc5)c5ccc(-c6cc7ccccc7c7ccccc67)cc5)cc4)ccc23)cc1 ; c1ccc(-c2cccc3cc(-c4ccc(N(c5ccc(-c6ccccc6-c6cccc7ccccc67)cc5)c5ccc6c(c5)oc5ccccc56)cc4)ccc23)cc1]

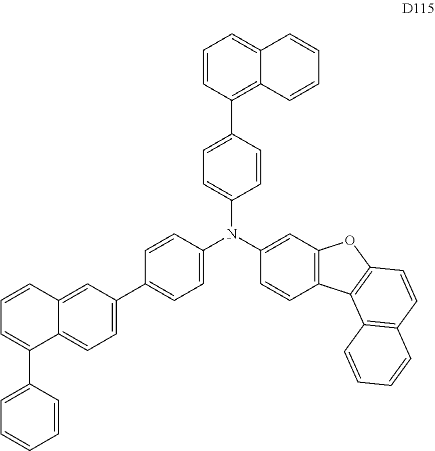 [SMILES: c1ccc(-c2cccc3cc(-c4ccc(N(c5ccc(-c6cccc7ccccc67)cc5)c5ccc6c(c5)oc5ccc7ccccc7c56)cc4)ccc23)cc1]

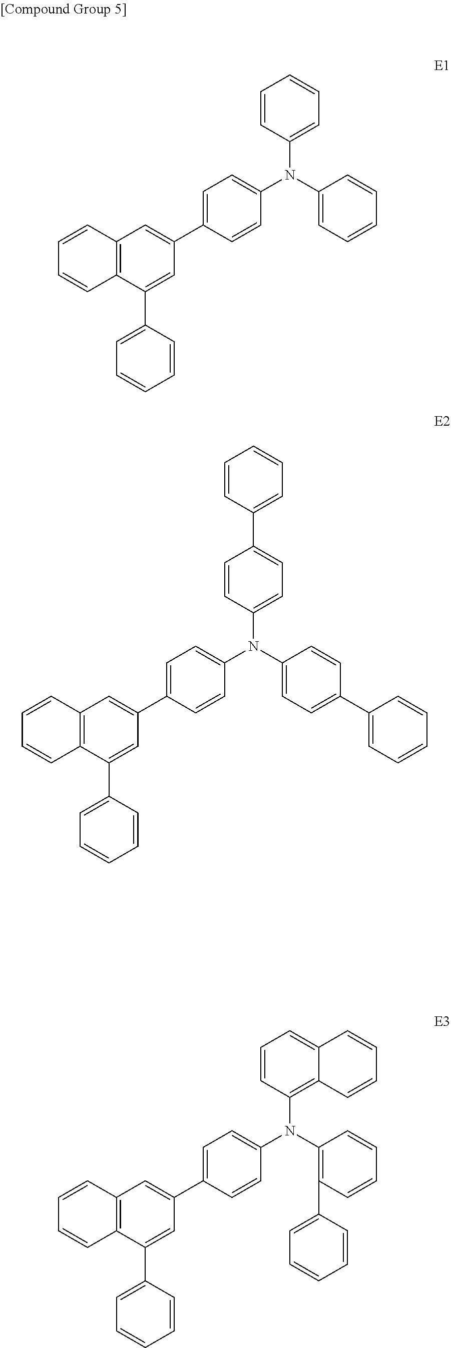 [SMILES: c1ccc(-c2cc(-c3ccc(N(c4ccccc4)c4ccccc4)cc3)cc3ccccc23)cc1 ; c1ccc(-c2ccc(N(c3ccc(-c4ccccc4)cc3)c3ccc(-c4cc(-c5ccccc5)c5ccccc5c4)cc3)cc2)cc1 ; c1ccc(-c2ccccc2N(c2ccc(-c3cc(-c4ccccc4)c4ccccc4c3)cc2)c2cccc3ccccc23)cc1]

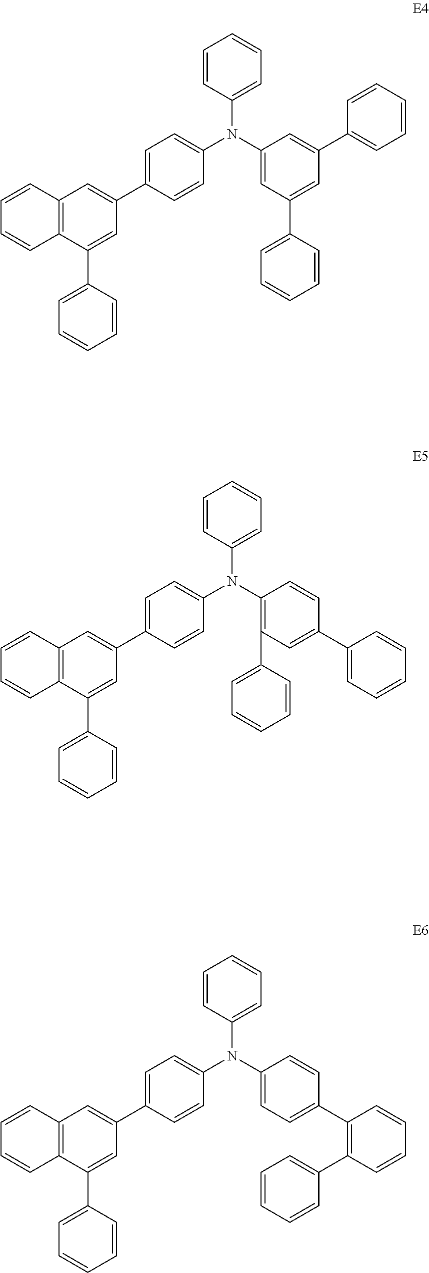 [SMILES: c1ccc(-c2cc(-c3ccccc3)cc(N(c3ccccc3)c3ccc(-c4cc(-c5ccccc5)c5ccccc5c4)cc3)c2)cc1 ; c1ccc(-c2ccc(N(c3ccccc3)c3ccc(-c4cc(-c5ccccc5)c5ccccc5c4)cc3)c(-c3ccccc3)c2)cc1 ; c1ccc(-c2ccccc2-c2ccc(N(c3ccccc3)c3ccc(-c4cc(-c5ccccc5)c5ccccc5c4)cc3)cc2)cc1]

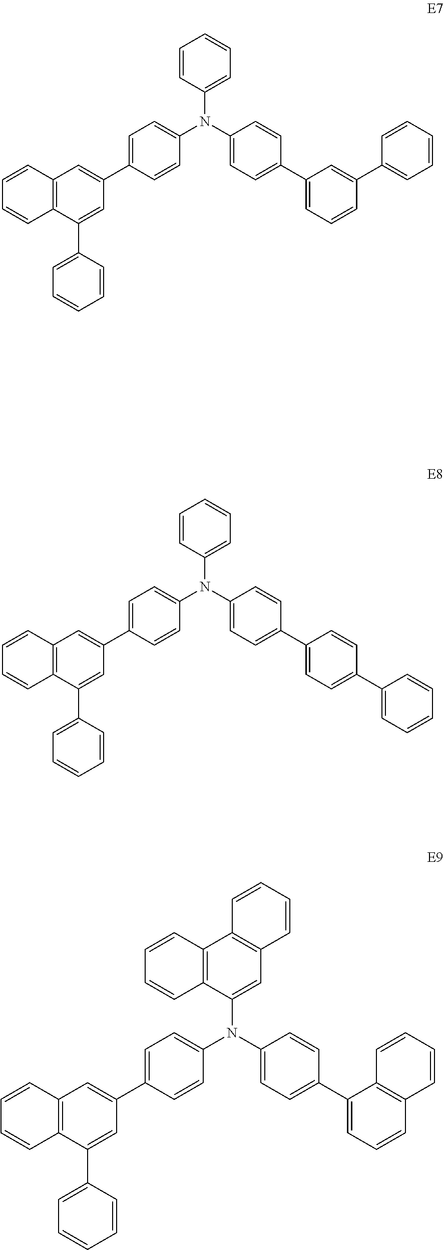 [SMILES: c1ccc(-c2cccc(-c3ccc(N(c4ccccc4)c4ccc(-c5cc(-c6ccccc6)c6ccccc6c5)cc4)cc3)c2)cc1 ; c1ccc(-c2ccc(-c3ccc(N(c4ccccc4)c4ccc(-c5cc(-c6ccccc6)c6ccccc6c5)cc4)cc3)cc2)cc1 ; c1ccc(-c2cc(-c3ccc(N(c4ccc(-c5cccc6ccccc56)cc4)c4cc5ccccc5c5ccccc45)cc3)cc3ccccc23)cc1]

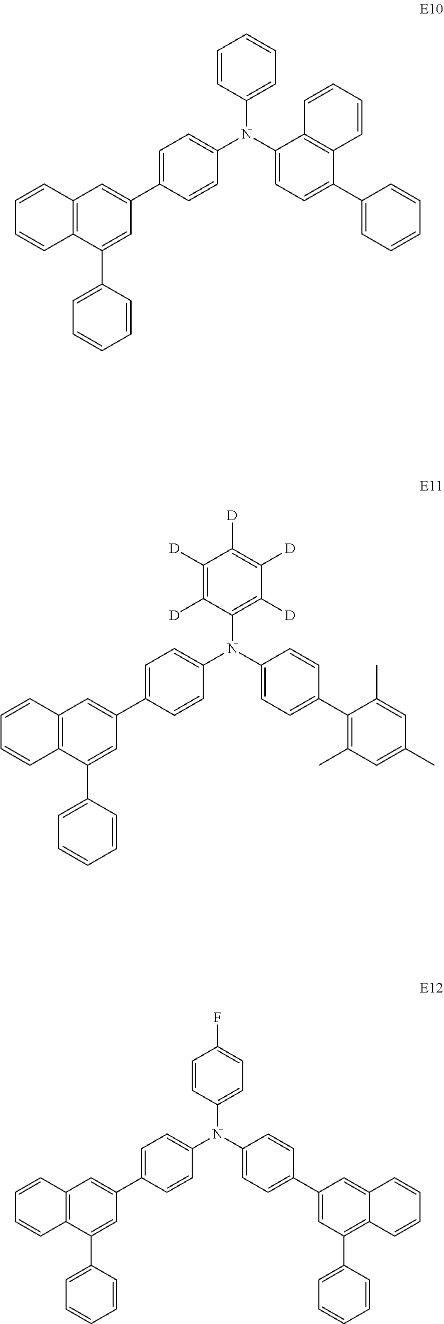 [SMILES: c1ccc(-c2cc(-c3ccc(N(c4ccccc4)c4ccc(-c5ccccc5)c5ccccc45)cc3)cc3ccccc23)cc1 ; [2H]c1c([2H])c([2H])c(N(c2ccc(-c3cc(-c4ccccc4)c4ccccc4c3)cc2)c2ccc(-c3c(C)cc(C)cc3C)cc2)c([2H])c1[2H] ; Fc1ccc(N(c2ccc(-c3cc(-c4ccccc4)c4ccccc4c3)cc2)c2ccc(-c3cc(-c4ccccc4)c4ccccc4c3)cc2)cc1]

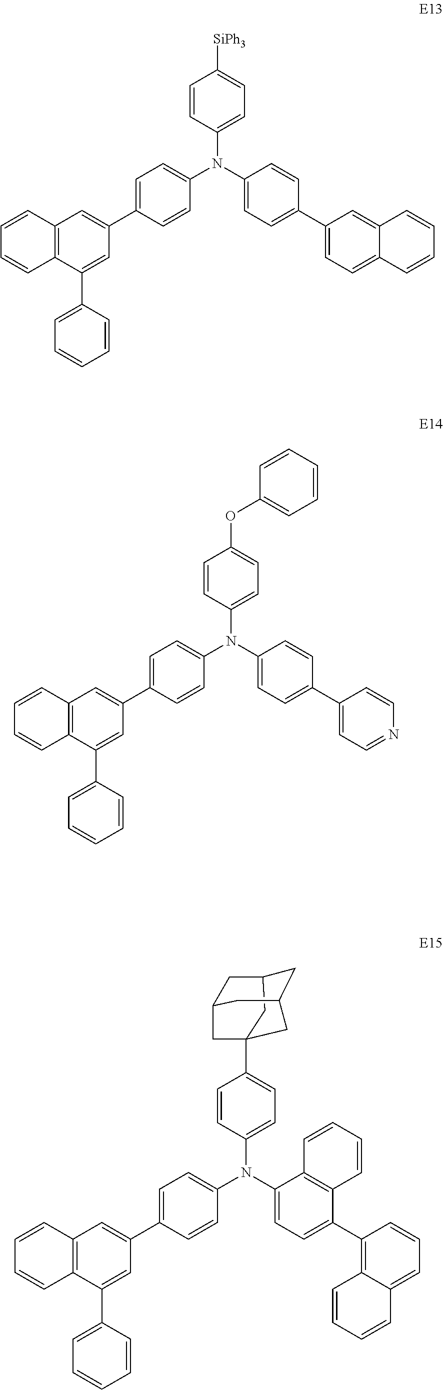

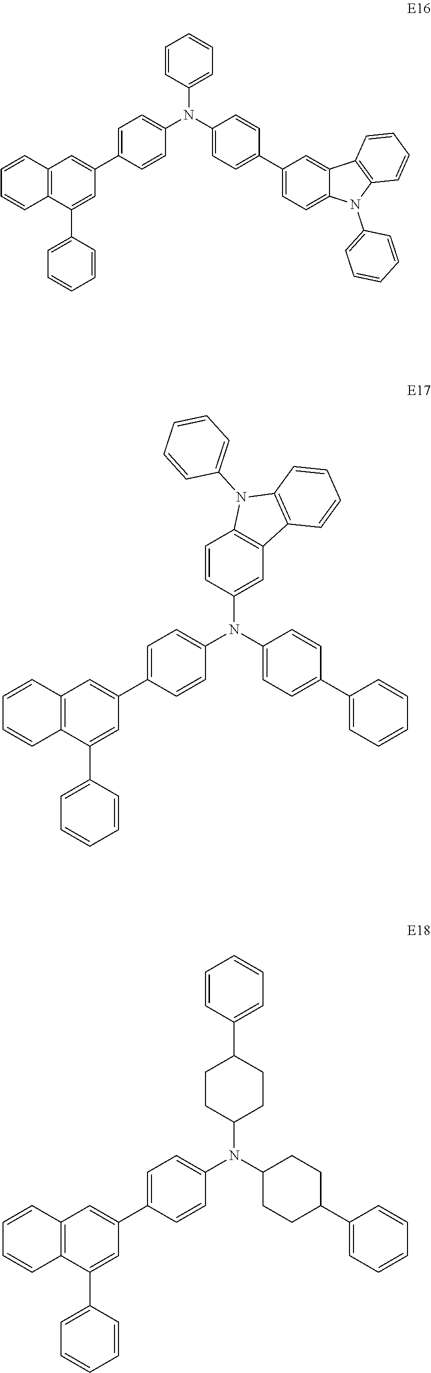 [SMILES: c1ccc(-c2cc(-c3ccc(N(c4ccccc4)c4ccc(-c5ccc6c(c5)c5ccccc5n6-c5ccccc5)cc4)cc3)cc3ccccc23)cc1 ; c1ccc(-c2ccc(N(c3ccc(-c4cc(-c5ccccc5)c5ccccc5c4)cc3)c3ccc4c(c3)c3ccccc3n4-c3ccccc3)cc2)cc1 ; c1ccc(-c2cc(-c3ccc(N(C4CCC(c5ccccc5)CC4)C4CCC(c5ccccc5)CC4)cc3)cc3ccccc23)cc1]

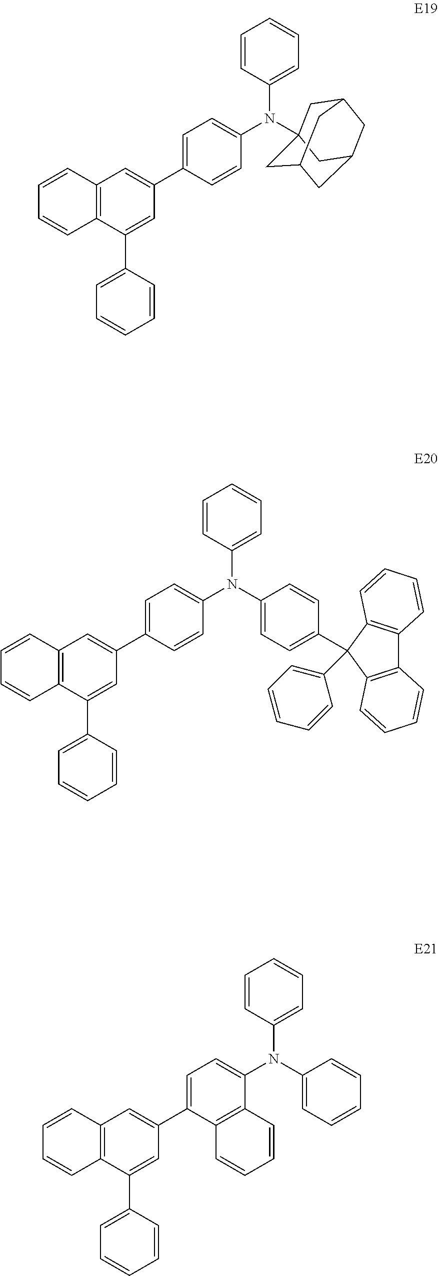 [SMILES: c1ccc(-c2cc(-c3ccc(N(c4ccccc4)C45CC6CC(CC(C6)C4)C5)cc3)cc3ccccc23)cc1 ; c1ccc(-c2cc(-c3ccc(N(c4ccccc4)c4ccc(C5(c6ccccc6)c6ccccc6-c6ccccc65)cc4)cc3)cc3ccccc23)cc1 ; c1ccc(-c2cc(-c3ccc(N(c4ccccc4)c4ccccc4)c4ccccc34)cc3ccccc23)cc1]

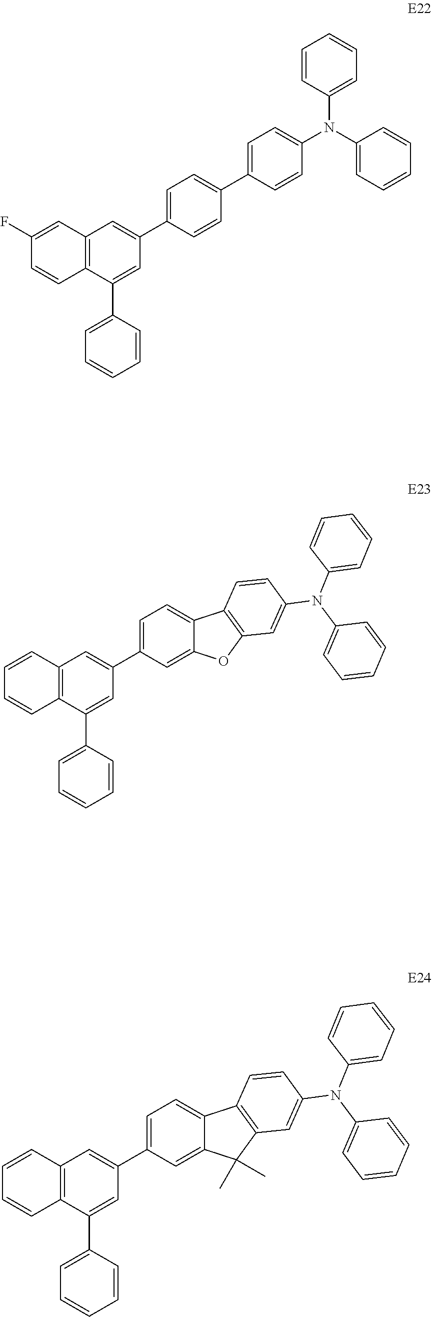 [SMILES: Fc1ccc2c(-c3ccccc3)cc(-c3ccc(-c4ccc(N(c5ccccc5)c5ccccc5)cc4)cc3)cc2c1 ; c1ccc(-c2cc(-c3ccc4c(c3)oc3cc(N(c5ccccc5)c5ccccc5)ccc34)cc3ccccc23)cc1 ; CC1(C)c2cc(-c3cc(-c4ccccc4)c4ccccc4c3)ccc2-c2ccc(N(c3ccccc3)c3ccccc3)cc21]

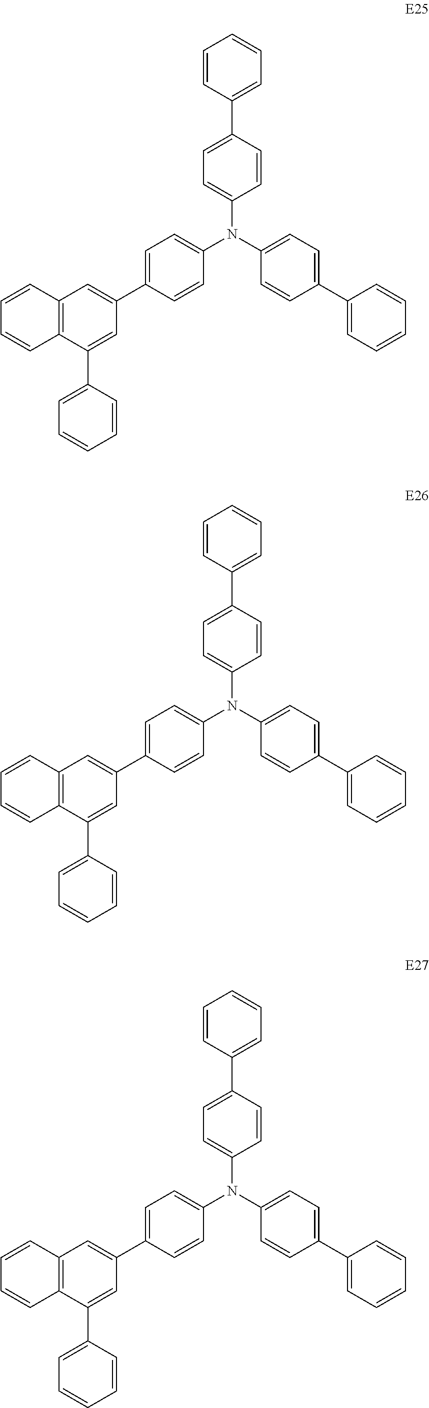 [SMILES: c1ccc(-c2ccc(N(c3ccc(-c4ccccc4)cc3)c3ccc(-c4cc(-c5ccccc5)c5ccccc5c4)cc3)cc2)cc1 ; c1ccc(-c2ccc(N(c3ccc(-c4ccccc4)cc3)c3ccc(-c4cc(-c5ccccc5)c5ccccc5c4)cc3)cc2)cc1 ; c1ccc(-c2ccc(N(c3ccc(-c4ccccc4)cc3)c3ccc(-c4cc(-c5ccccc5)c5ccccc5c4)cc3)cc2)cc1]

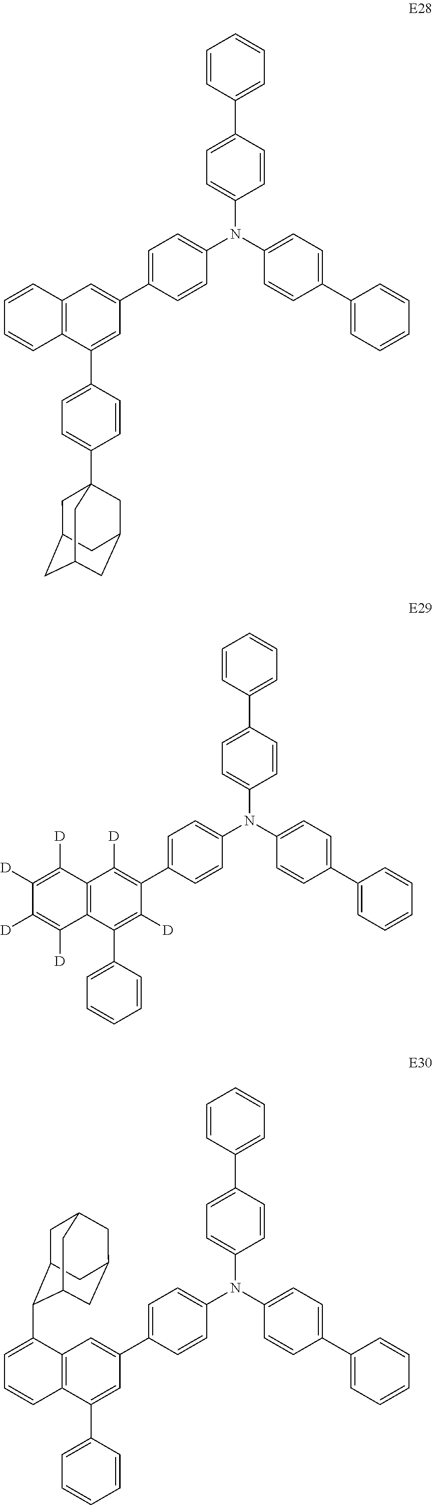 [SMILES: c1ccc(-c2ccc(N(c3ccc(-c4ccccc4)cc3)c3ccc(-c4cc(-c5ccc(C67CC8CC(CC(C8)C6)C7)cc5)c5ccccc5c4)cc3)cc2)cc1 ; [2H]c1c([2H])c([2H])c2c(-c3ccccc3)c([2H])c(-c3ccc(N(c4ccc(-c5ccccc5)cc4)c4ccc(-c5ccccc5)cc4)cc3)c([2H])c2c1[2H] ; c1ccc(-c2ccc(N(c3ccc(-c4ccccc4)cc3)c3ccc(-c4cc(-c5ccccc5)c5cccc(C6C7CC8CC(C7)CC6C8)c5c4)cc3)cc2)cc1]

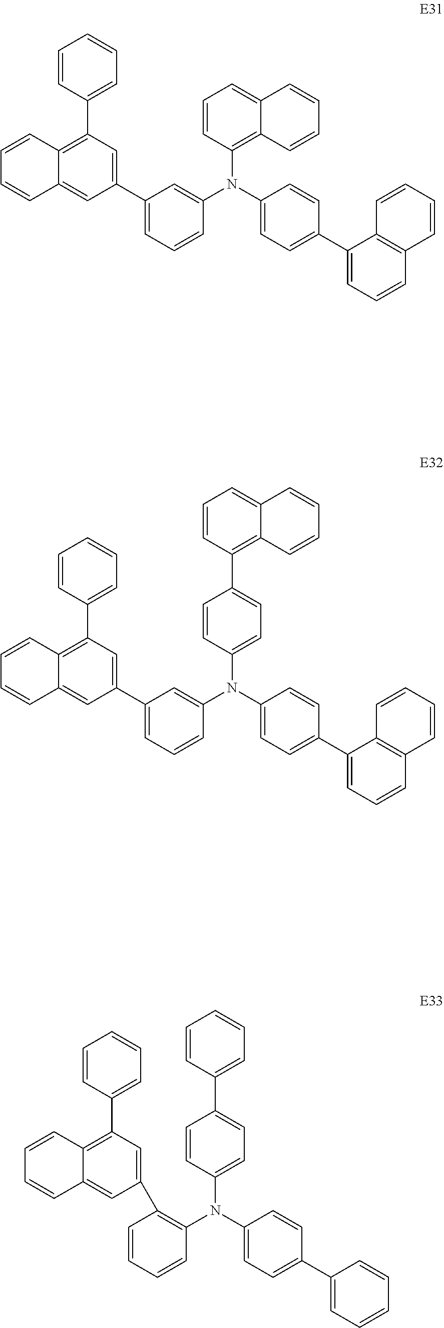 [SMILES: c1ccc(-c2cc(-c3cccc(N(c4ccc(-c5cccc6ccccc56)cc4)c4cccc5ccccc45)c3)cc3ccccc23)cc1 ; c1ccc(-c2cc(-c3cccc(N(c4ccc(-c5cccc6ccccc56)cc4)c4ccc(-c5cccc6ccccc56)cc4)c3)cc3ccccc23)cc1 ; c1ccc(-c2ccc(N(c3ccc(-c4ccccc4)cc3)c3ccccc3-c3cc(-c4ccccc4)c4ccccc4c3)cc2)cc1]

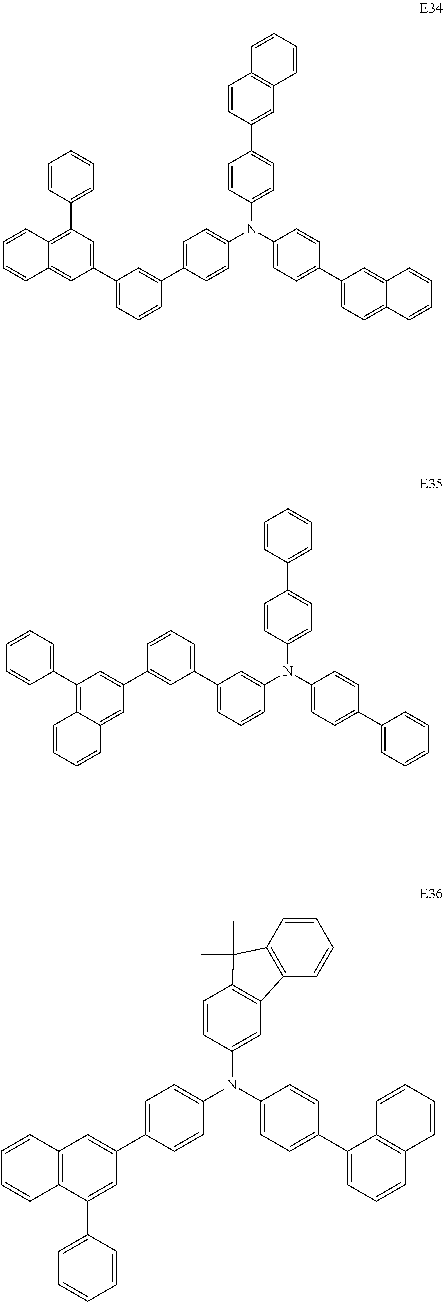 [SMILES: c1ccc(-c2cc(-c3cccc(-c4ccc(N(c5ccc(-c6ccc7ccccc7c6)cc5)c5ccc(-c6ccc7ccccc7c6)cc5)cc4)c3)cc3ccccc23)cc1 ; c1ccc(-c2ccc(N(c3ccc(-c4ccccc4)cc3)c3cccc(-c4cccc(-c5cc(-c6ccccc6)c6ccccc6c5)c4)c3)cc2)cc1 ; CC1(C)c2ccccc2-c2cc(N(c3ccc(-c4cc(-c5ccccc5)c5ccccc5c4)cc3)c3ccc(-c4cccc5ccccc45)cc3)ccc21]

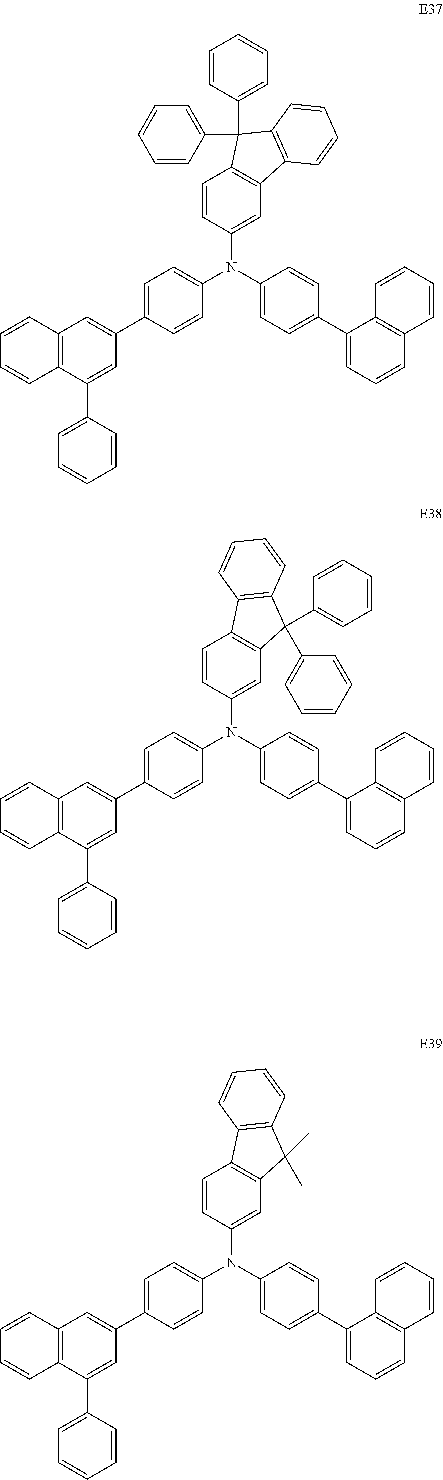 [SMILES: c1ccc(-c2cc(-c3ccc(N(c4ccc(-c5cccc6ccccc56)cc4)c4ccc5c(c4)-c4ccccc4C5(c4ccccc4)c4ccccc4)cc3)cc3ccccc23)cc1 ; c1ccc(-c2cc(-c3ccc(N(c4ccc(-c5cccc6ccccc56)cc4)c4ccc5c(c4)C(c4ccccc4)(c4ccccc4)c4ccccc4-5)cc3)cc3ccccc23)cc1 ; CC1(C)c2ccccc2-c2ccc(N(c3ccc(-c4cc(-c5ccccc5)c5ccccc5c4)cc3)c3ccc(-c4cccc5ccccc45)cc3)cc21]

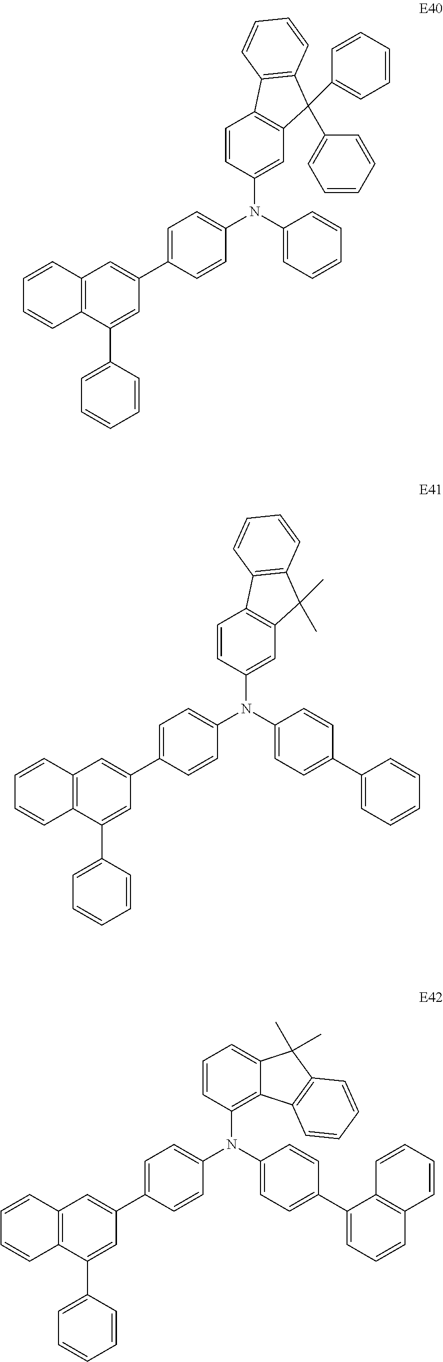 [SMILES: c1ccc(-c2cc(-c3ccc(N(c4ccccc4)c4ccc5c(c4)C(c4ccccc4)(c4ccccc4)c4ccccc4-5)cc3)cc3ccccc23)cc1 ; CC1(C)c2ccccc2-c2ccc(N(c3ccc(-c4ccccc4)cc3)c3ccc(-c4cc(-c5ccccc5)c5ccccc5c4)cc3)cc21 ; CC1(C)c2ccccc2-c2c(N(c3ccc(-c4cc(-c5ccccc5)c5ccccc5c4)cc3)c3ccc(-c4cccc5ccccc45)cc3)cccc21]

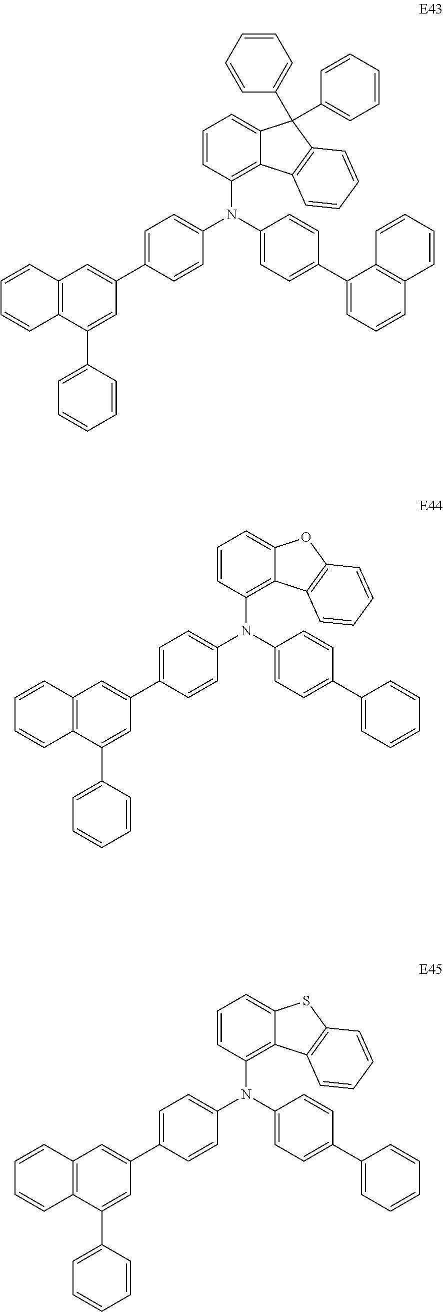 [SMILES: c1ccc(-c2cc(-c3ccc(N(c4ccc(-c5cccc6ccccc56)cc4)c4cccc5c4-c4ccccc4C5(c4ccccc4)c4ccccc4)cc3)cc3ccccc23)cc1 ; c1ccc(-c2ccc(N(c3ccc(-c4cc(-c5ccccc5)c5ccccc5c4)cc3)c3cccc4oc5ccccc5c34)cc2)cc1 ; c1ccc(-c2ccc(N(c3ccc(-c4cc(-c5ccccc5)c5ccccc5c4)cc3)c3cccc4sc5ccccc5c34)cc2)cc1]

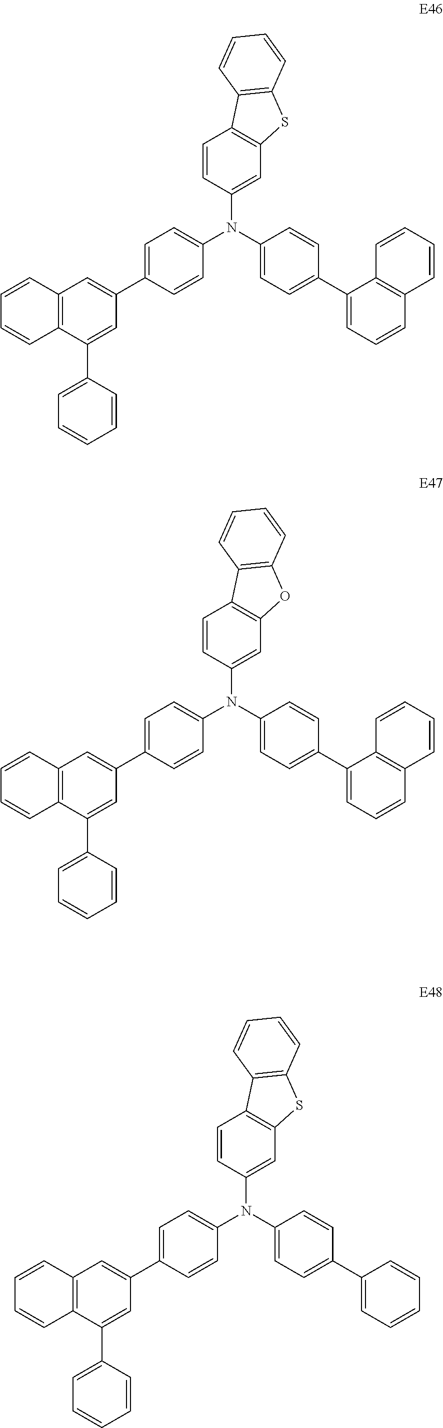 [SMILES: c1ccc(-c2cc(-c3ccc(N(c4ccc(-c5cccc6ccccc56)cc4)c4ccc5c(c4)sc4ccccc45)cc3)cc3ccccc23)cc1 ; c1ccc(-c2cc(-c3ccc(N(c4ccc(-c5cccc6ccccc56)cc4)c4ccc5c(c4)oc4ccccc45)cc3)cc3ccccc23)cc1 ; c1ccc(-c2ccc(N(c3ccc(-c4cc(-c5ccccc5)c5ccccc5c4)cc3)c3ccc4c(c3)sc3ccccc34)cc2)cc1]

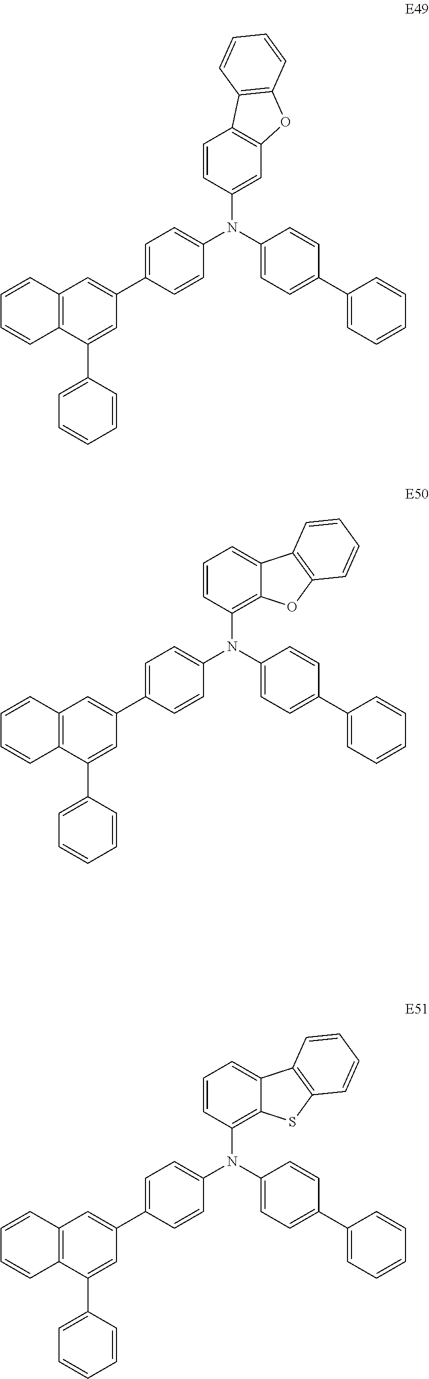 [SMILES: c1ccc(-c2ccc(N(c3ccc(-c4cc(-c5ccccc5)c5ccccc5c4)cc3)c3ccc4c(c3)oc3ccccc34)cc2)cc1 ; c1ccc(-c2ccc(N(c3ccc(-c4cc(-c5ccccc5)c5ccccc5c4)cc3)c3cccc4c3oc3ccccc34)cc2)cc1 ; c1ccc(-c2ccc(N(c3ccc(-c4cc(-c5ccccc5)c5ccccc5c4)cc3)c3cccc4c3sc3ccccc34)cc2)cc1]

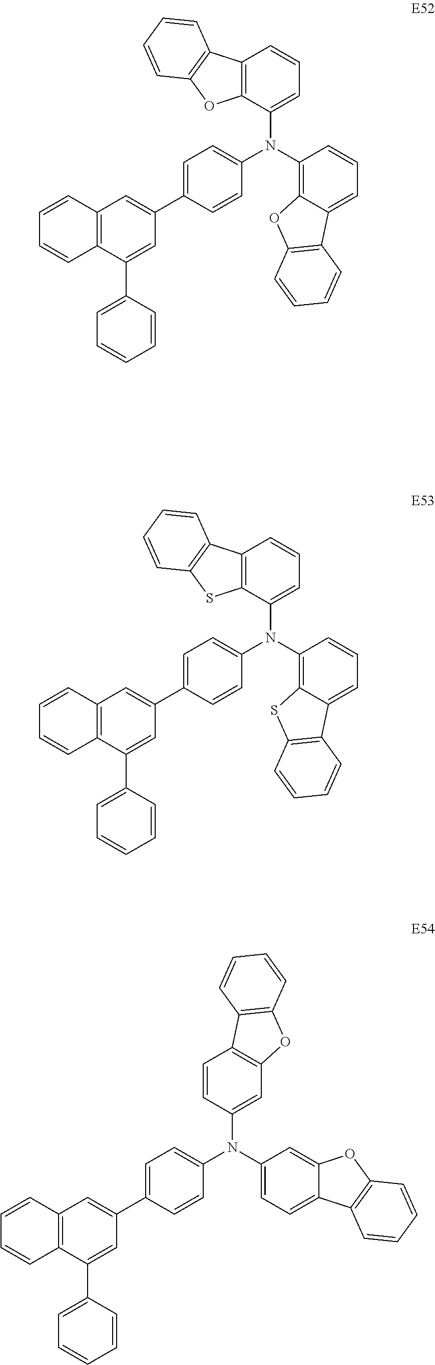 [SMILES: c1ccc(-c2cc(-c3ccc(N(c4cccc5c4oc4ccccc45)c4cccc5c4oc4ccccc45)cc3)cc3ccccc23)cc1 ; c1ccc(-c2cc(-c3ccc(N(c4cccc5c4sc4ccccc45)c4cccc5c4sc4ccccc45)cc3)cc3ccccc23)cc1 ; c1ccc(-c2cc(-c3ccc(N(c4ccc5c(c4)oc4ccccc45)c4ccc5c(c4)oc4ccccc45)cc3)cc3ccccc23)cc1]

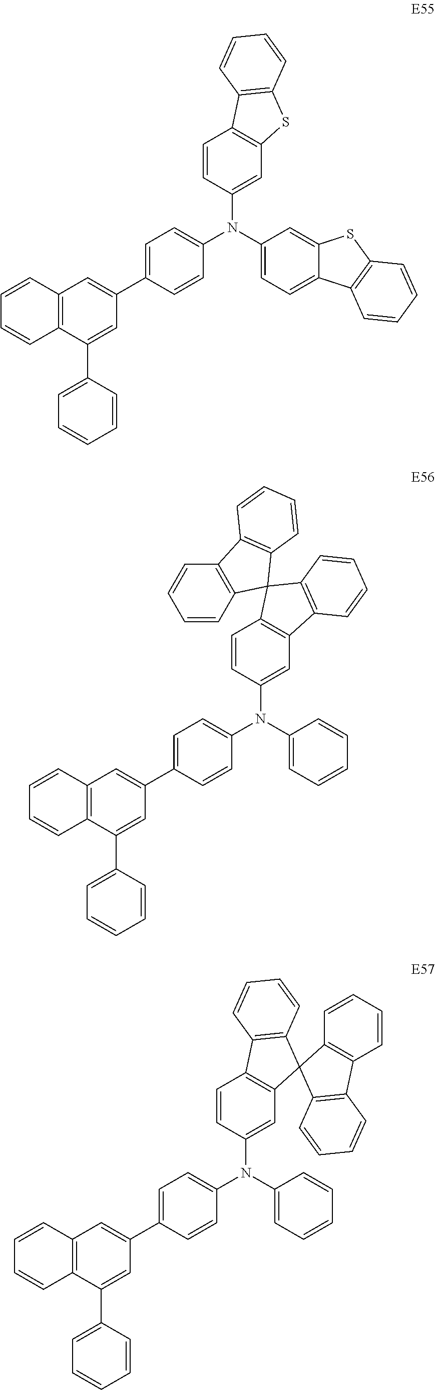 [SMILES: c1ccc(-c2cc(-c3ccc(N(c4ccc5c(c4)sc4ccccc45)c4ccc5c(c4)sc4ccccc45)cc3)cc3ccccc23)cc1 ; c1ccc(-c2cc(-c3ccc(N(c4ccccc4)c4ccc5c(c4)-c4ccccc4C54c5ccccc5-c5ccccc54)cc3)cc3ccccc23)cc1 ; c1ccc(-c2cc(-c3ccc(N(c4ccccc4)c4ccc5c(c4)C4(c6ccccc6-c6ccccc64)c4ccccc4-5)cc3)cc3ccccc23)cc1]

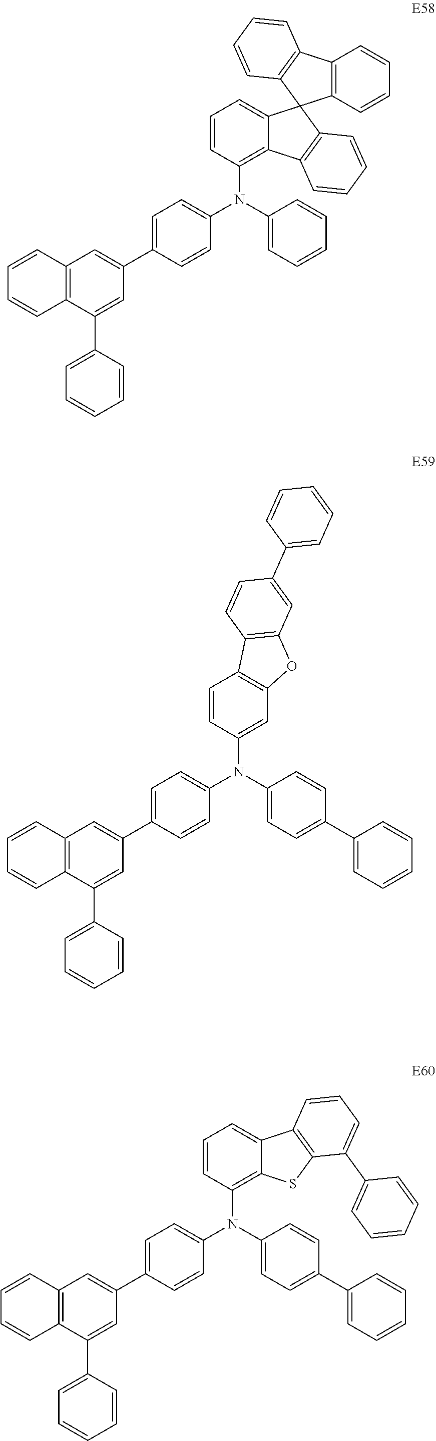 [SMILES: c1ccc(-c2cc(-c3ccc(N(c4ccccc4)c4cccc5c4-c4ccccc4C54c5ccccc5-c5ccccc54)cc3)cc3ccccc23)cc1 ; c1ccc(-c2ccc(N(c3ccc(-c4cc(-c5ccccc5)c5ccccc5c4)cc3)c3ccc4c(c3)oc3cc(-c5ccccc5)ccc34)cc2)cc1 ; c1ccc(-c2ccc(N(c3ccc(-c4cc(-c5ccccc5)c5ccccc5c4)cc3)c3cccc4c3sc3c(-c5ccccc5)cccc34)cc2)cc1]

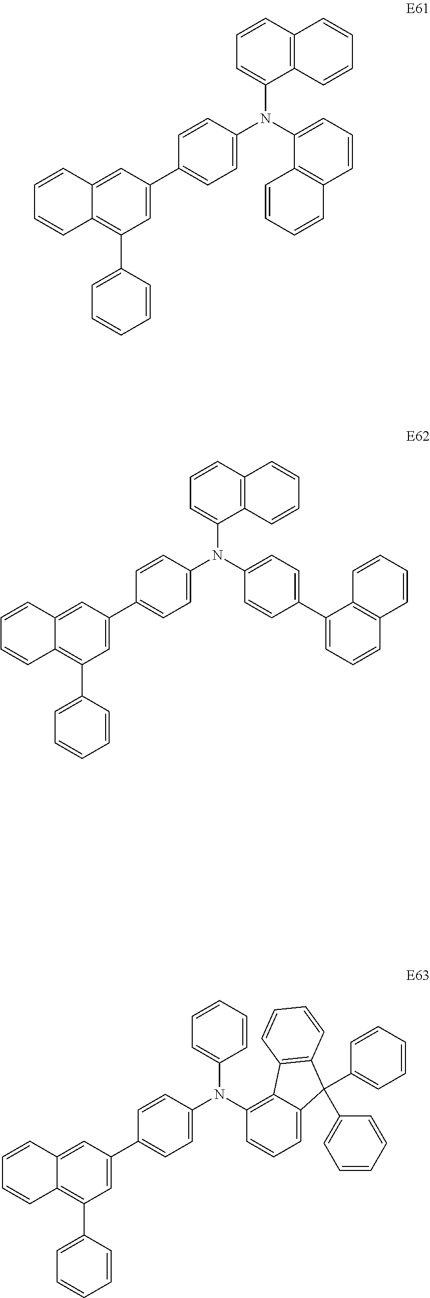 [SMILES: c1ccc(-c2cc(-c3ccc(N(c4cccc5ccccc45)c4cccc5ccccc45)cc3)cc3ccccc23)cc1 ; c1ccc(-c2cc(-c3ccc(N(c4ccc(-c5cccc6ccccc56)cc4)c4cccc5ccccc45)cc3)cc3ccccc23)cc1 ; c1ccc(-c2cc(-c3ccc(N(c4ccccc4)c4cccc5c4-c4ccccc4C5(c4ccccc4)c4ccccc4)cc3)cc3ccccc23)cc1]

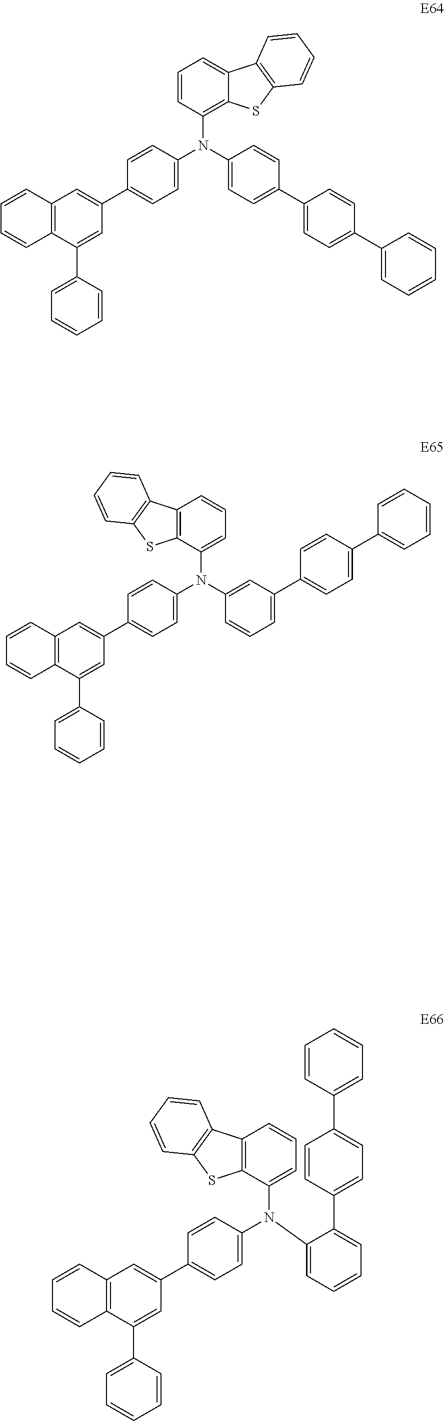 [SMILES: c1ccc(-c2ccc(-c3ccc(N(c4ccc(-c5cc(-c6ccccc6)c6ccccc6c5)cc4)c4cccc5c4sc4ccccc45)cc3)cc2)cc1 ; c1ccc(-c2ccc(-c3cccc(N(c4ccc(-c5cc(-c6ccccc6)c6ccccc6c5)cc4)c4cccc5c4sc4ccccc45)c3)cc2)cc1 ; c1ccc(-c2ccc(-c3ccccc3N(c3ccc(-c4cc(-c5ccccc5)c5ccccc5c4)cc3)c3cccc4c3sc3ccccc34)cc2)cc1]

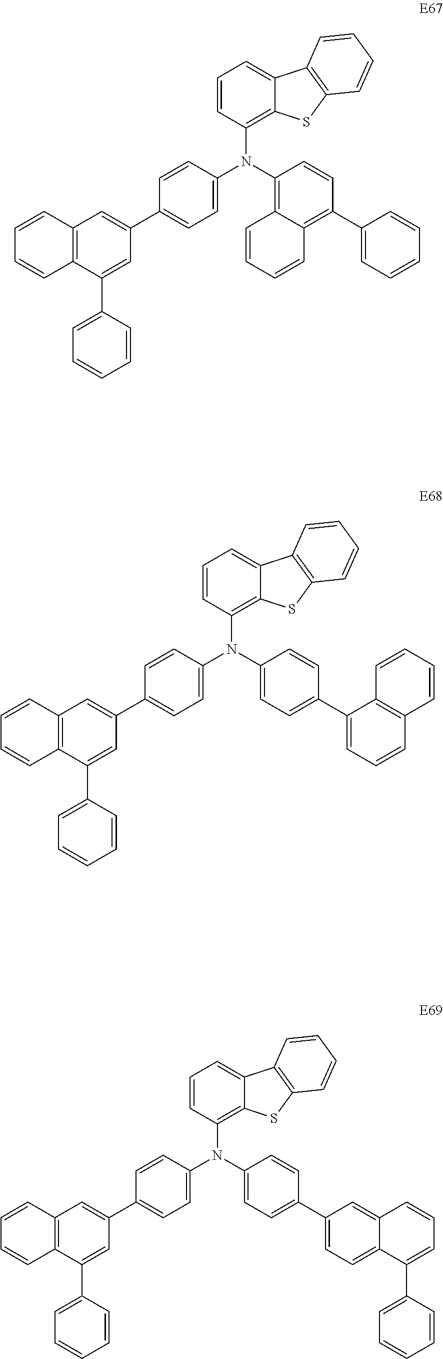 [SMILES: c1ccc(-c2cc(-c3ccc(N(c4ccc(-c5ccccc5)c5ccccc45)c4cccc5c4sc4ccccc45)cc3)cc3ccccc23)cc1 ; c1ccc(-c2cc(-c3ccc(N(c4ccc(-c5cccc6ccccc56)cc4)c4cccc5c4sc4ccccc45)cc3)cc3ccccc23)cc1 ; c1ccc(-c2cccc3cc(-c4ccc(N(c5ccc(-c6cc(-c7ccccc7)c7ccccc7c6)cc5)c5cccc6c5sc5ccccc56)cc4)ccc23)cc1]

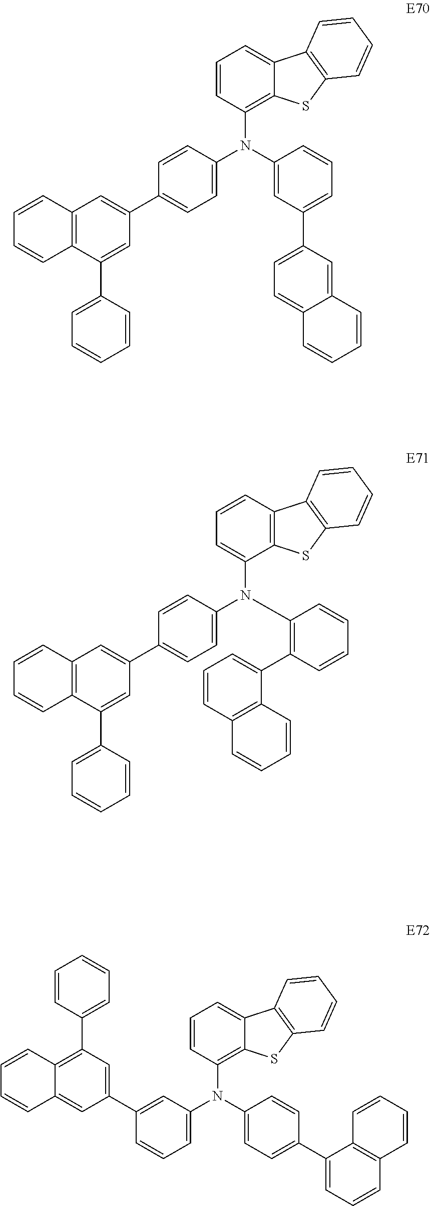 [SMILES: c1ccc(-c2cc(-c3ccc(N(c4cccc(-c5ccc6ccccc6c5)c4)c4cccc5c4sc4ccccc45)cc3)cc3ccccc23)cc1 ; c1ccc(-c2cc(-c3ccc(N(c4ccccc4-c4cccc5ccccc45)c4cccc5c4sc4ccccc45)cc3)cc3ccccc23)cc1 ; c1ccc(-c2cc(-c3cccc(N(c4ccc(-c5cccc6ccccc56)cc4)c4cccc5c4sc4ccccc45)c3)cc3ccccc23)cc1]

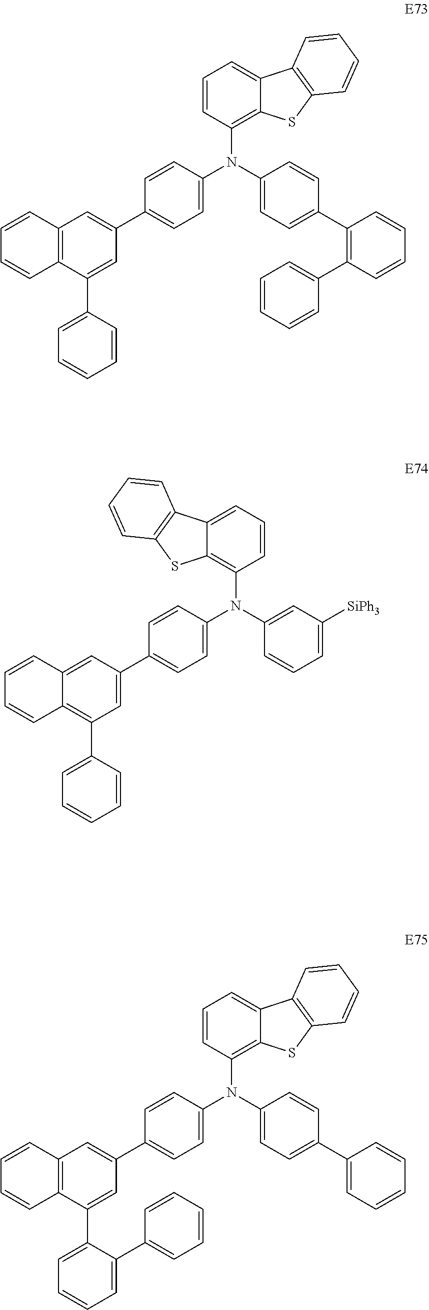

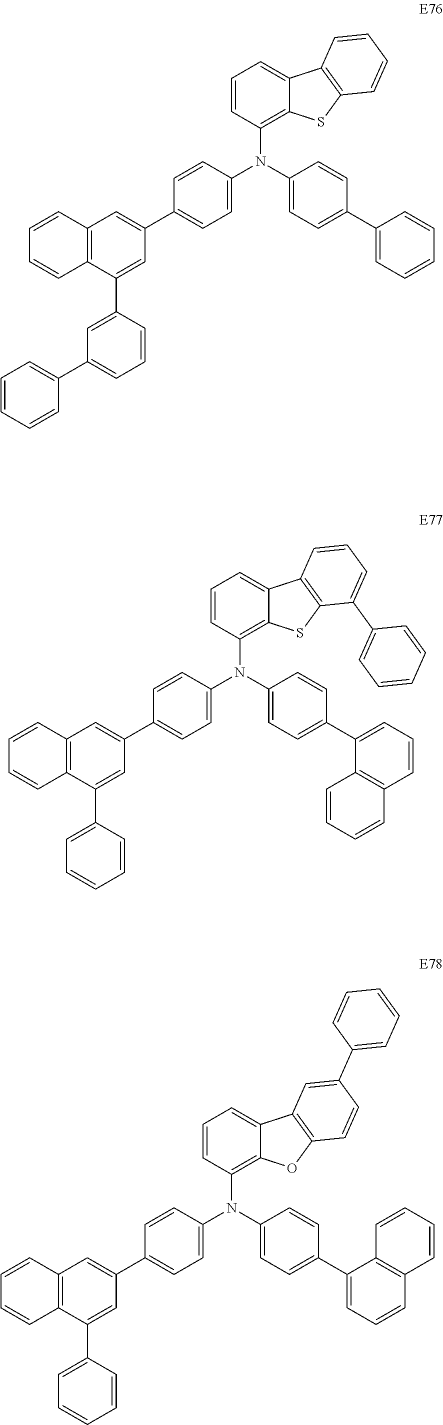 [SMILES: c1ccc(-c2ccc(N(c3ccc(-c4cc(-c5cccc(-c6ccccc6)c5)c5ccccc5c4)cc3)c3cccc4c3sc3ccccc34)cc2)cc1 ; c1ccc(-c2cc(-c3ccc(N(c4ccc(-c5cccc6ccccc56)cc4)c4cccc5c4sc4c(-c6ccccc6)cccc45)cc3)cc3ccccc23)cc1 ; c1ccc(-c2ccc3oc4c(N(c5ccc(-c6cc(-c7ccccc7)c7ccccc7c6)cc5)c5ccc(-c6cccc7ccccc67)cc5)cccc4c3c2)cc1]

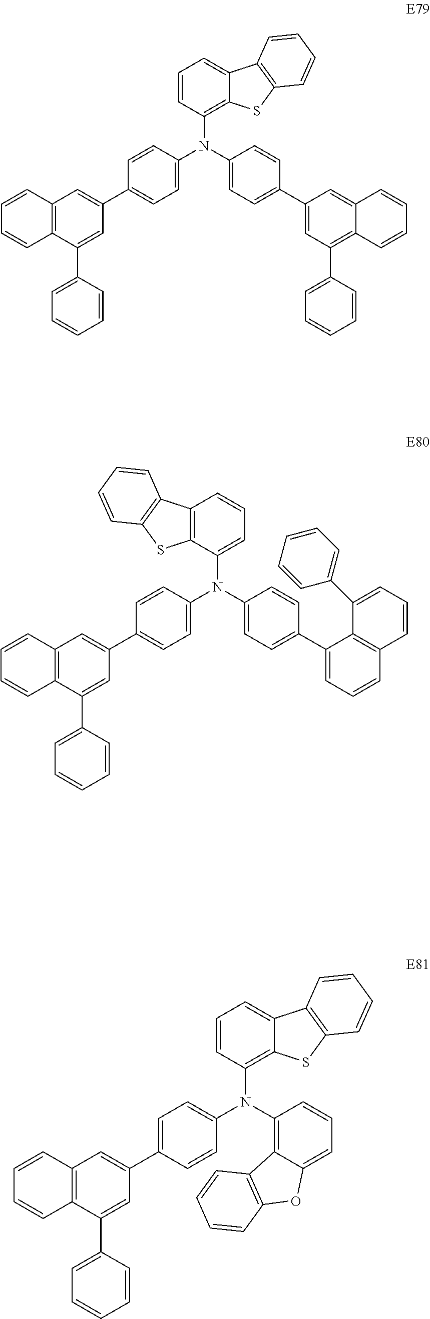 [SMILES: c1ccc(-c2cc(-c3ccc(N(c4ccc(-c5cc(-c6ccccc6)c6ccccc6c5)cc4)c4cccc5c4sc4ccccc45)cc3)cc3ccccc23)cc1 ; c1ccc(-c2cc(-c3ccc(N(c4ccc(-c5cccc6cccc(-c7ccccc7)c56)cc4)c4cccc5c4sc4ccccc45)cc3)cc3ccccc23)cc1 ; c1ccc(-c2cc(-c3ccc(N(c4cccc5c4sc4ccccc45)c4cccc5oc6ccccc6c45)cc3)cc3ccccc23)cc1]

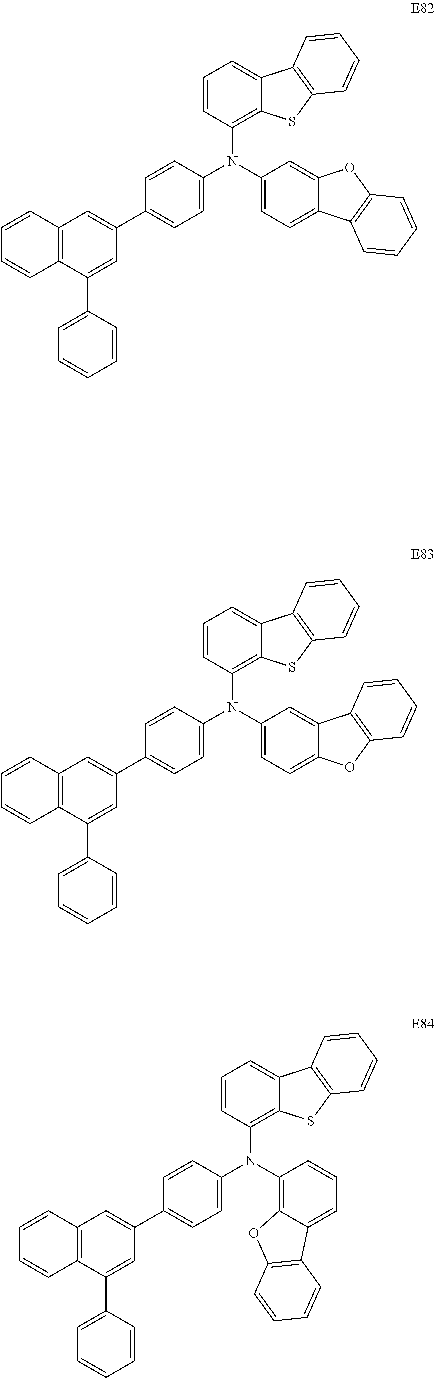 [SMILES: c1ccc(-c2cc(-c3ccc(N(c4ccc5c(c4)oc4ccccc45)c4cccc5c4sc4ccccc45)cc3)cc3ccccc23)cc1 ; c1ccc(-c2cc(-c3ccc(N(c4ccc5oc6ccccc6c5c4)c4cccc5c4sc4ccccc45)cc3)cc3ccccc23)cc1 ; c1ccc(-c2cc(-c3ccc(N(c4cccc5c4oc4ccccc45)c4cccc5c4sc4ccccc45)cc3)cc3ccccc23)cc1]

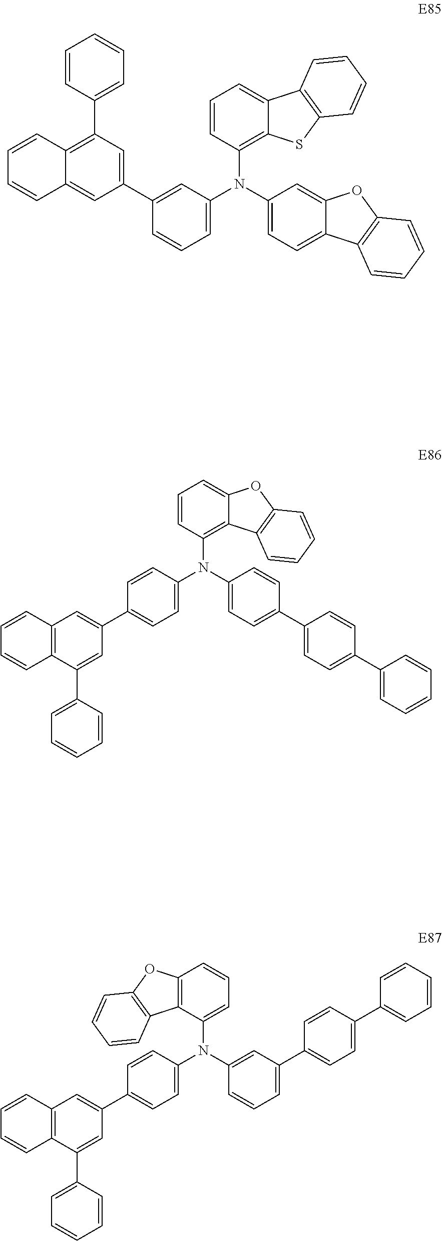 [SMILES: c1ccc(-c2cc(-c3cccc(N(c4ccc5c(c4)oc4ccccc45)c4cccc5c4sc4ccccc45)c3)cc3ccccc23)cc1 ; c1ccc(-c2ccc(-c3ccc(N(c4ccc(-c5cc(-c6ccccc6)c6ccccc6c5)cc4)c4cccc5oc6ccccc6c45)cc3)cc2)cc1 ; c1ccc(-c2ccc(-c3cccc(N(c4ccc(-c5cc(-c6ccccc6)c6ccccc6c5)cc4)c4cccc5oc6ccccc6c45)c3)cc2)cc1]

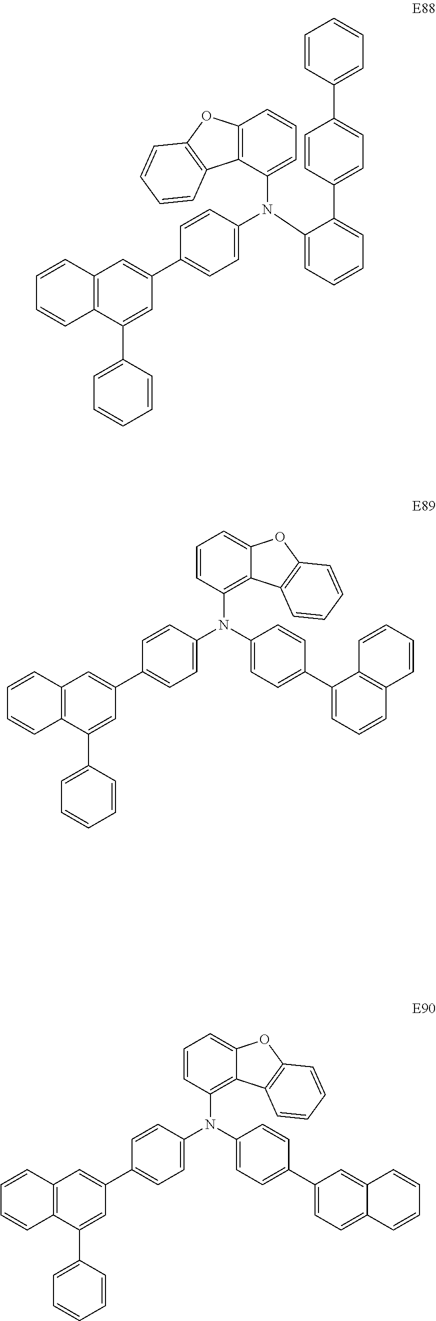 [SMILES: c1ccc(-c2ccc(-c3ccccc3N(c3ccc(-c4cc(-c5ccccc5)c5ccccc5c4)cc3)c3cccc4oc5ccccc5c34)cc2)cc1 ; c1ccc(-c2cc(-c3ccc(N(c4ccc(-c5cccc6ccccc56)cc4)c4cccc5oc6ccccc6c45)cc3)cc3ccccc23)cc1 ; c1ccc(-c2cc(-c3ccc(N(c4ccc(-c5ccc6ccccc6c5)cc4)c4cccc5oc6ccccc6c45)cc3)cc3ccccc23)cc1]

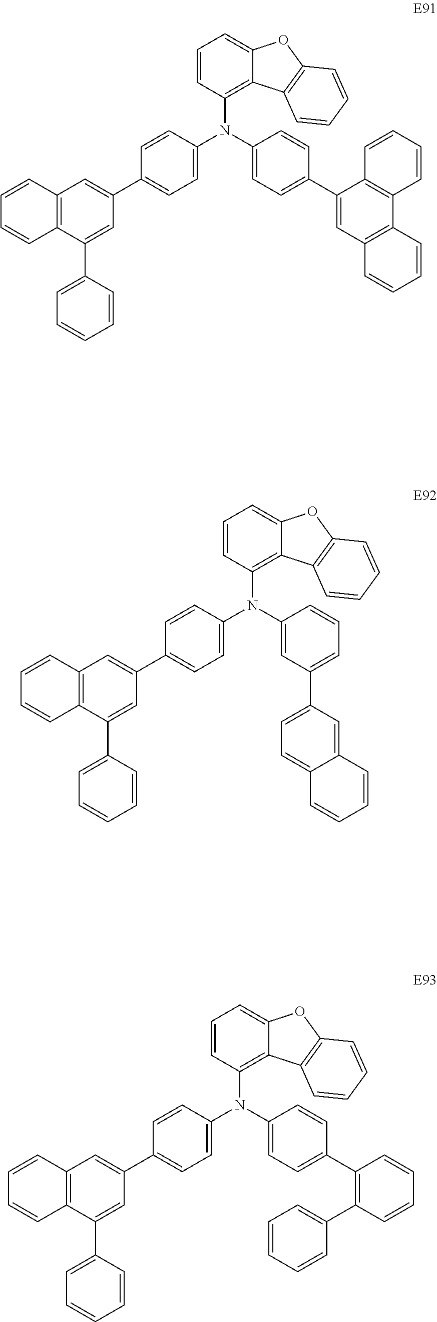 [SMILES: c1ccc(-c2cc(-c3ccc(N(c4ccc(-c5cc6ccccc6c6ccccc56)cc4)c4cccc5oc6ccccc6c45)cc3)cc3ccccc23)cc1 ; c1ccc(-c2cc(-c3ccc(N(c4cccc(-c5ccc6ccccc6c5)c4)c4cccc5oc6ccccc6c45)cc3)cc3ccccc23)cc1 ; c1ccc(-c2ccccc2-c2ccc(N(c3ccc(-c4cc(-c5ccccc5)c5ccccc5c4)cc3)c3cccc4oc5ccccc5c34)cc2)cc1]

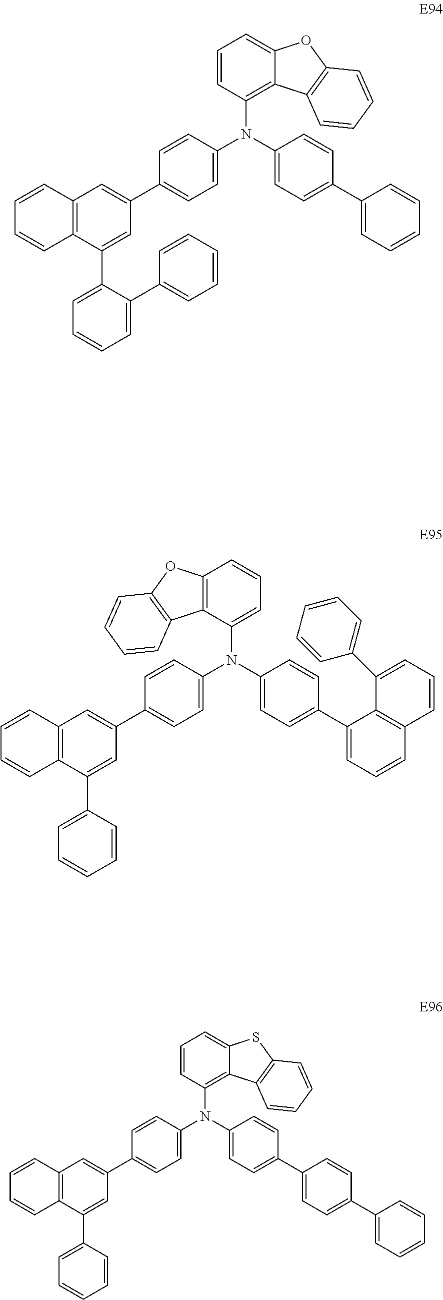 [SMILES: c1ccc(-c2ccc(N(c3ccc(-c4cc(-c5ccccc5-c5ccccc5)c5ccccc5c4)cc3)c3cccc4oc5ccccc5c34)cc2)cc1 ; c1ccc(-c2cc(-c3ccc(N(c4ccc(-c5cccc6cccc(-c7ccccc7)c56)cc4)c4cccc5oc6ccccc6c45)cc3)cc3ccccc23)cc1 ; c1ccc(-c2ccc(-c3ccc(N(c4ccc(-c5cc(-c6ccccc6)c6ccccc6c5)cc4)c4cccc5sc6ccccc6c45)cc3)cc2)cc1]

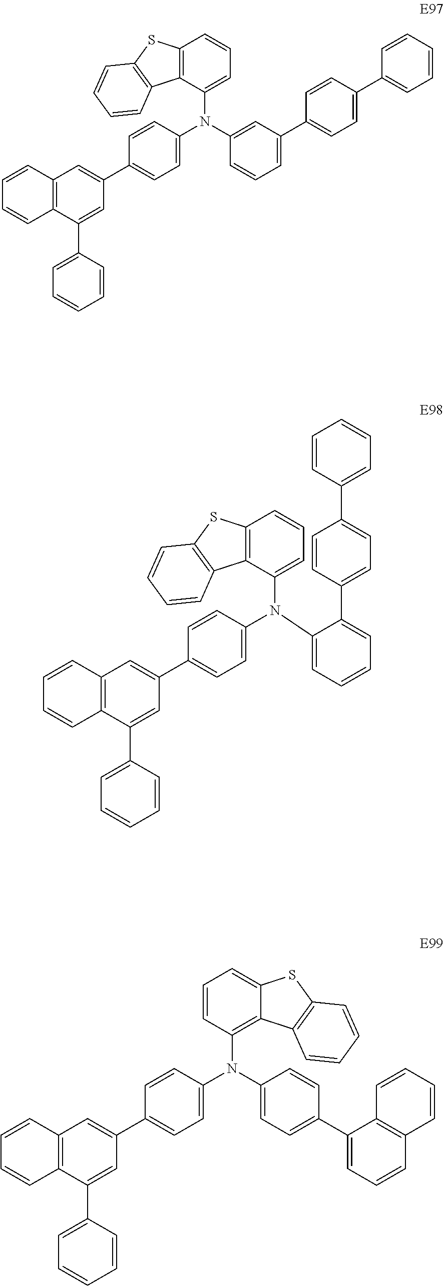 [SMILES: c1ccc(-c2ccc(-c3cccc(N(c4ccc(-c5cc(-c6ccccc6)c6ccccc6c5)cc4)c4cccc5sc6ccccc6c45)c3)cc2)cc1 ; c1ccc(-c2ccc(-c3ccccc3N(c3ccc(-c4cc(-c5ccccc5)c5ccccc5c4)cc3)c3cccc4sc5ccccc5c34)cc2)cc1 ; c1ccc(-c2cc(-c3ccc(N(c4ccc(-c5cccc6ccccc56)cc4)c4cccc5sc6ccccc6c45)cc3)cc3ccccc23)cc1]

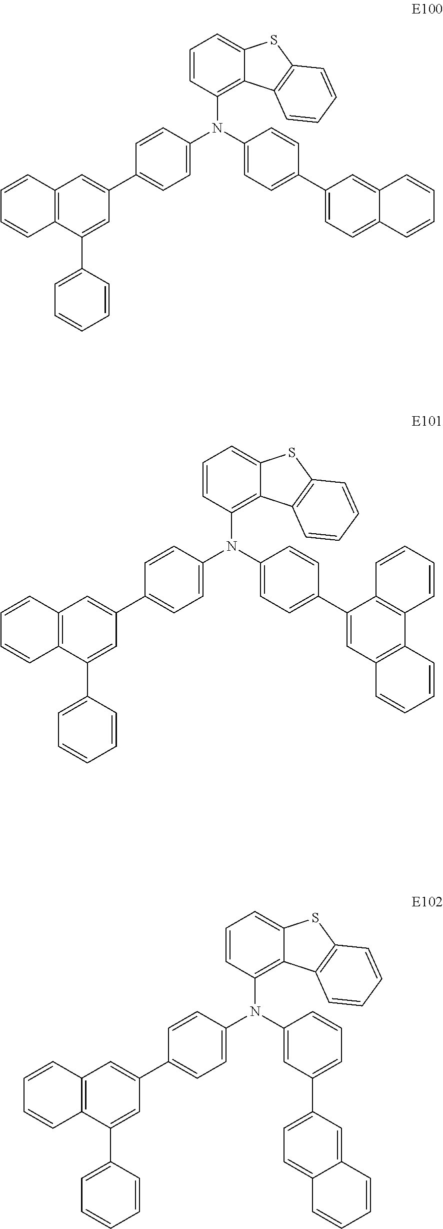 [SMILES: c1ccc(-c2cc(-c3ccc(N(c4ccc(-c5ccc6ccccc6c5)cc4)c4cccc5sc6ccccc6c45)cc3)cc3ccccc23)cc1 ; c1ccc(-c2cc(-c3ccc(N(c4ccc(-c5cc6ccccc6c6ccccc56)cc4)c4cccc5sc6ccccc6c45)cc3)cc3ccccc23)cc1 ; c1ccc(-c2cc(-c3ccc(N(c4cccc(-c5ccc6ccccc6c5)c4)c4cccc5sc6ccccc6c45)cc3)cc3ccccc23)cc1]

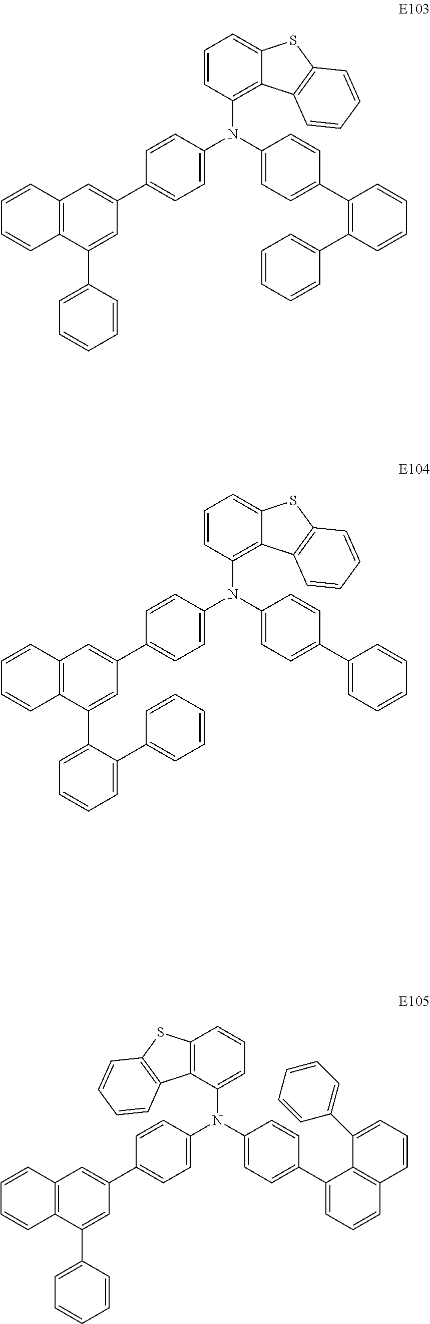 [SMILES: c1ccc(-c2ccccc2-c2ccc(N(c3ccc(-c4cc(-c5ccccc5)c5ccccc5c4)cc3)c3cccc4sc5ccccc5c34)cc2)cc1 ; c1ccc(-c2ccc(N(c3ccc(-c4cc(-c5ccccc5-c5ccccc5)c5ccccc5c4)cc3)c3cccc4sc5ccccc5c34)cc2)cc1 ; c1ccc(-c2cc(-c3ccc(N(c4ccc(-c5cccc6cccc(-c7ccccc7)c56)cc4)c4cccc5sc6ccccc6c45)cc3)cc3ccccc23)cc1]

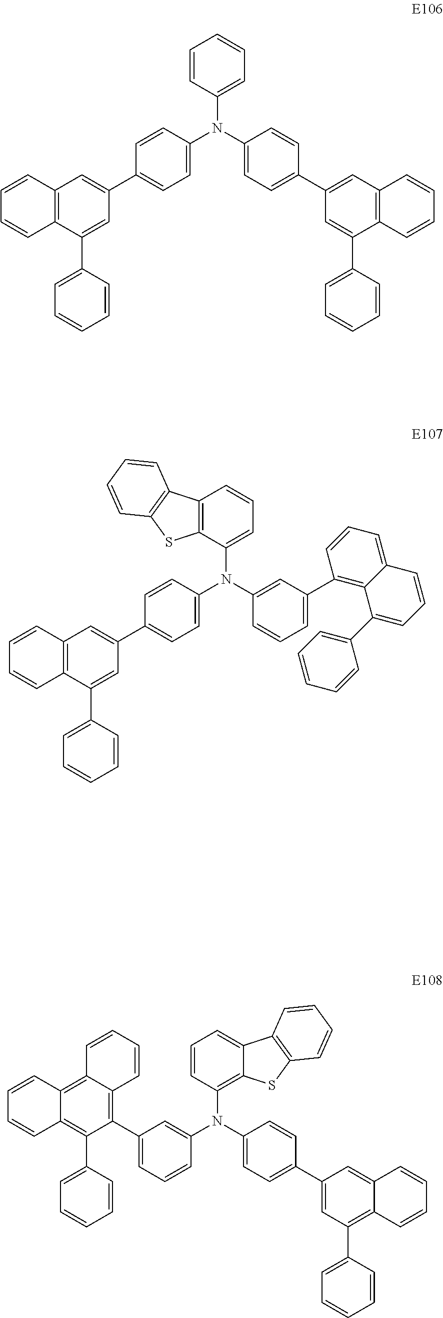 [SMILES: c1ccc(-c2cc(-c3ccc(N(c4ccccc4)c4ccc(-c5cc(-c6ccccc6)c6ccccc6c5)cc4)cc3)cc3ccccc23)cc1 ; c1ccc(-c2cc(-c3ccc(N(c4cccc(-c5cccc6cccc(-c7ccccc7)c56)c4)c4cccc5c4sc4ccccc45)cc3)cc3ccccc23)cc1 ; c1ccc(-c2cc(-c3ccc(N(c4cccc(-c5c(-c6ccccc6)c6ccccc6c6ccccc56)c4)c4cccc5c4sc4ccccc45)cc3)cc3ccccc23)cc1]

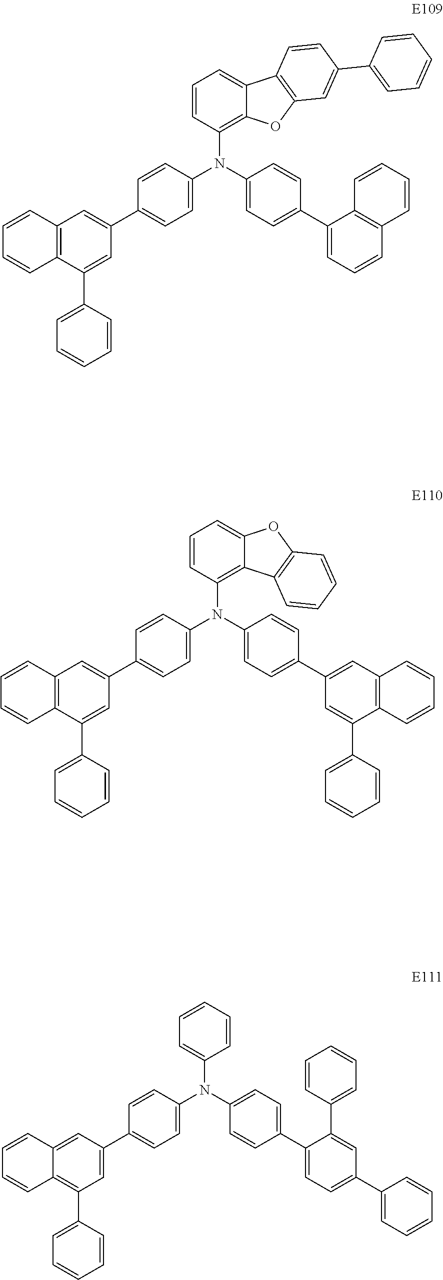 [SMILES: c1ccc(-c2ccc3c(c2)oc2c(N(c4ccc(-c5cc(-c6ccccc6)c6ccccc6c5)cc4)c4ccc(-c5cccc6ccccc56)cc4)cccc23)cc1 ; c1ccc(-c2cc(-c3ccc(N(c4ccc(-c5cc(-c6ccccc6)c6ccccc6c5)cc4)c4cccc5oc6ccccc6c45)cc3)cc3ccccc23)cc1 ; c1ccc(-c2ccc(-c3ccc(N(c4ccccc4)c4ccc(-c5cc(-c6ccccc6)c6ccccc6c5)cc4)cc3)c(-c3ccccc3)c2)cc1]

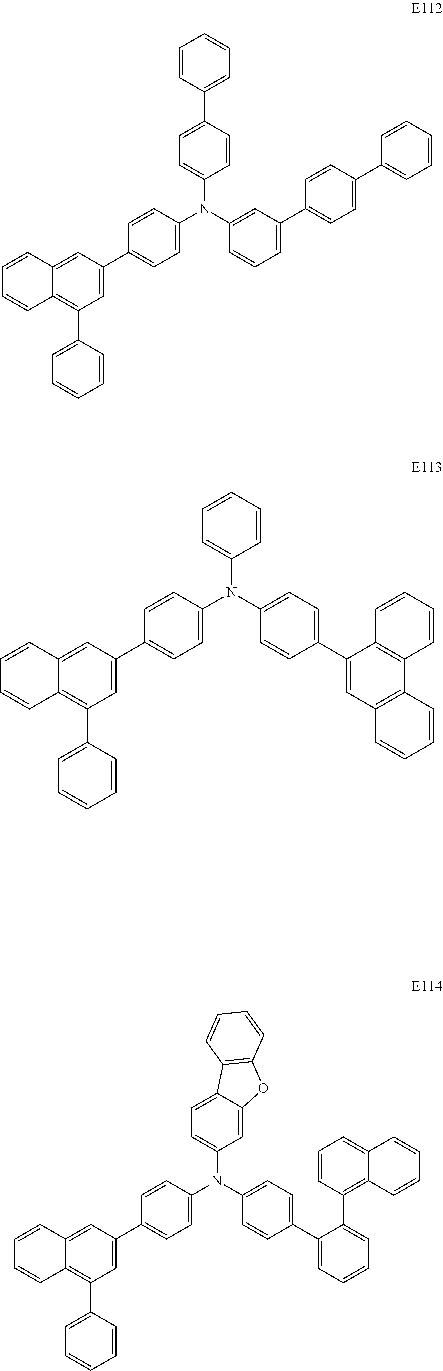 [SMILES: c1ccc(-c2ccc(-c3cccc(N(c4ccc(-c5ccccc5)cc4)c4ccc(-c5cc(-c6ccccc6)c6ccccc6c5)cc4)c3)cc2)cc1 ; c1ccc(-c2cc(-c3ccc(N(c4ccccc4)c4ccc(-c5cc6ccccc6c6ccccc56)cc4)cc3)cc3ccccc23)cc1 ; c1ccc(-c2cc(-c3ccc(N(c4ccc(-c5ccccc5-c5cccc6ccccc56)cc4)c4ccc5c(c4)oc4ccccc45)cc3)cc3ccccc23)cc1]

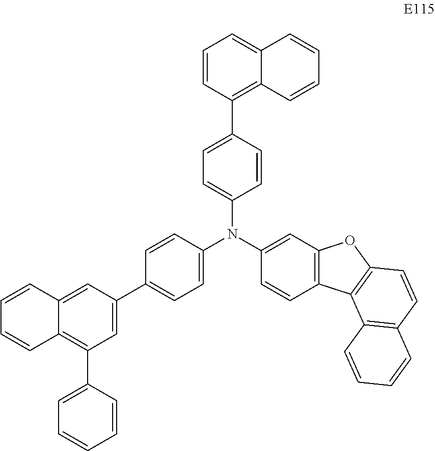 [SMILES: c1ccc(-c2cc(-c3ccc(N(c4ccc(-c5cccc6ccccc56)cc4)c4ccc5c(c4)oc4ccc6ccccc6c45)cc3)cc3ccccc23)cc1]

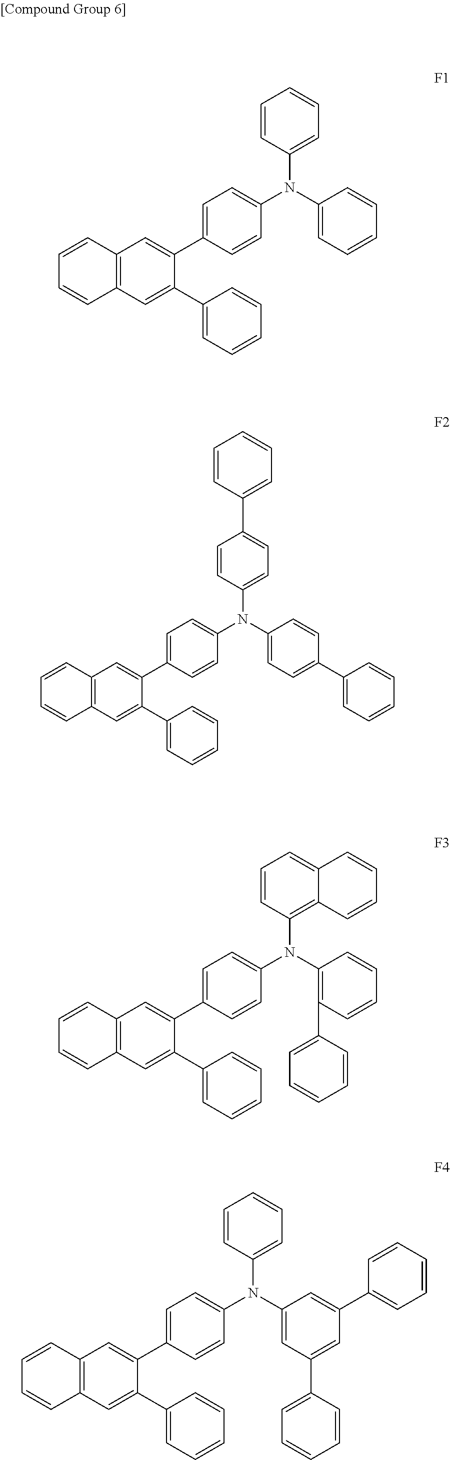 [SMILES: c1ccc(-c2cc3ccccc3cc2-c2ccc(N(c3ccccc3)c3ccccc3)cc2)cc1 ; c1ccc(-c2ccc(N(c3ccc(-c4ccccc4)cc3)c3ccc(-c4cc5ccccc5cc4-c4ccccc4)cc3)cc2)cc1 ; c1ccc(-c2cc3ccccc3cc2-c2ccc(N(c3ccccc3-c3ccccc3)c3cccc4ccccc34)cc2)cc1 ; c1ccc(-c2cc(-c3ccccc3)cc(N(c3ccccc3)c3ccc(-c4cc5ccccc5cc4-c4ccccc4)cc3)c2)cc1]

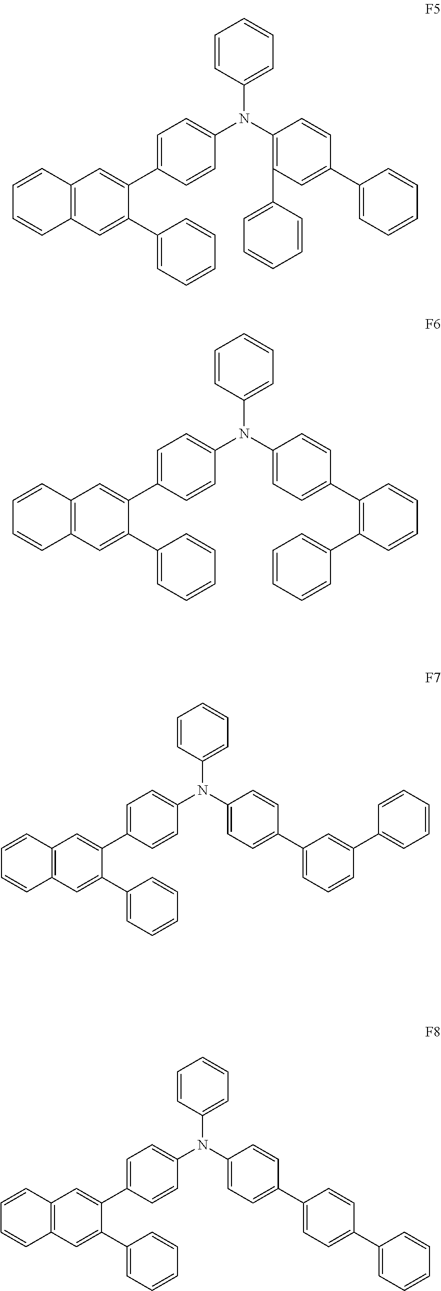 [SMILES: c1ccc(-c2ccc(N(c3ccccc3)c3ccc(-c4cc5ccccc5cc4-c4ccccc4)cc3)c(-c3ccccc3)c2)cc1 ; c1ccc(-c2ccccc2-c2ccc(N(c3ccccc3)c3ccc(-c4cc5ccccc5cc4-c4ccccc4)cc3)cc2)cc1 ; c1ccc(-c2cccc(-c3ccc(N(c4ccccc4)c4ccc(-c5cc6ccccc6cc5-c5ccccc5)cc4)cc3)c2)cc1 ; c1ccc(-c2ccc(-c3ccc(N(c4ccccc4)c4ccc(-c5cc6ccccc6cc5-c5ccccc5)cc4)cc3)cc2)cc1]

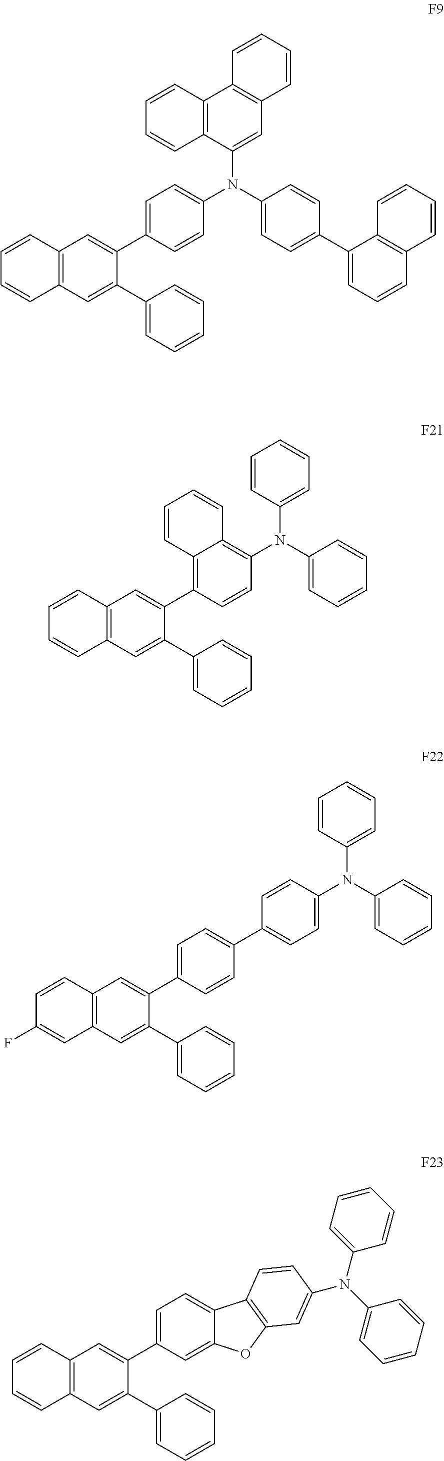 [SMILES: c1ccc(-c2cc3ccccc3cc2-c2ccc(N(c3ccc(-c4cccc5ccccc45)cc3)c3cc4ccccc4c4ccccc34)cc2)cc1 ; c1ccc(-c2cc3ccccc3cc2-c2ccc(N(c3ccccc3)c3ccccc3)c3ccccc23)cc1 ; Fc1ccc2cc(-c3ccc(-c4ccc(N(c5ccccc5)c5ccccc5)cc4)cc3)c(-c3ccccc3)cc2c1 ; c1ccc(-c2cc3ccccc3cc2-c2ccc3c(c2)oc2cc(N(c4ccccc4)c4ccccc4)ccc23)cc1]

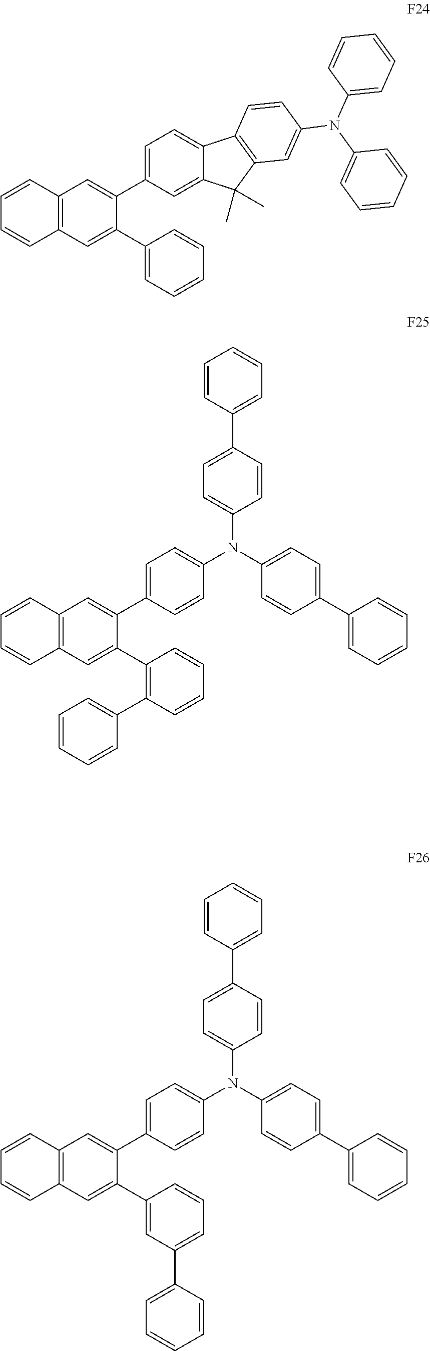 [SMILES: CC1(C)c2cc(-c3cc4ccccc4cc3-c3ccccc3)ccc2-c2ccc(N(c3ccccc3)c3ccccc3)cc21 ; c1ccc(-c2ccc(N(c3ccc(-c4ccccc4)cc3)c3ccc(-c4cc5ccccc5cc4-c4ccccc4-c4ccccc4)cc3)cc2)cc1 ; c1ccc(-c2ccc(N(c3ccc(-c4ccccc4)cc3)c3ccc(-c4cc5ccccc5cc4-c4cccc(-c5ccccc5)c4)cc3)cc2)cc1]

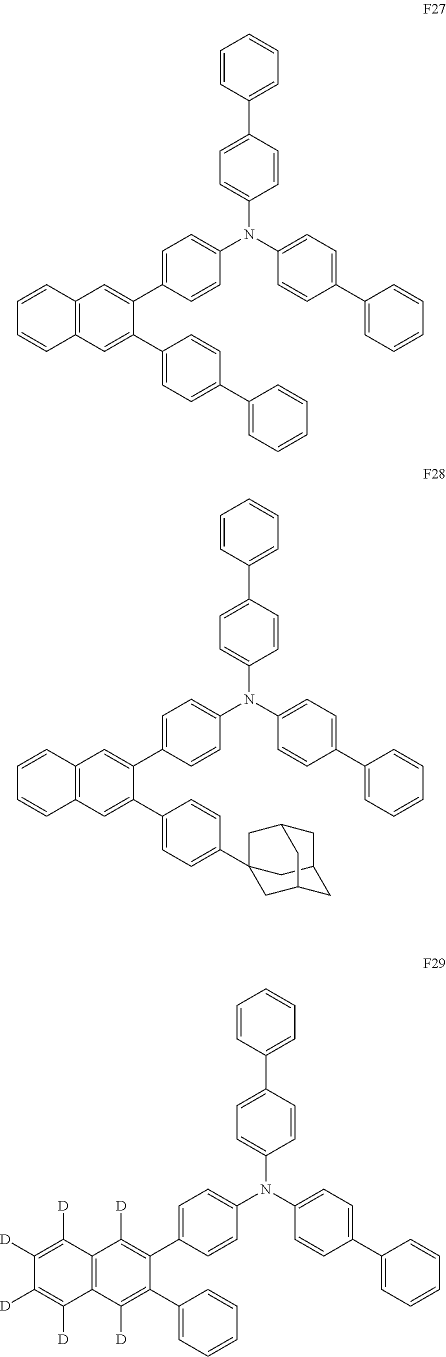 [SMILES: c1ccc(-c2ccc(-c3cc4ccccc4cc3-c3ccc(N(c4ccc(-c5ccccc5)cc4)c4ccc(-c5ccccc5)cc4)cc3)cc2)cc1 ; c1ccc(-c2ccc(N(c3ccc(-c4ccccc4)cc3)c3ccc(-c4cc5ccccc5cc4-c4ccc(C56CC7CC(CC(C7)C5)C6)cc4)cc3)cc2)cc1 ; [2H]c1c([2H])c([2H])c2c([2H])c(-c3ccc(N(c4ccc(-c5ccccc5)cc4)c4ccc(-c5ccccc5)cc4)cc3)c(-c3ccccc3)c([2H])c2c1[2H]]

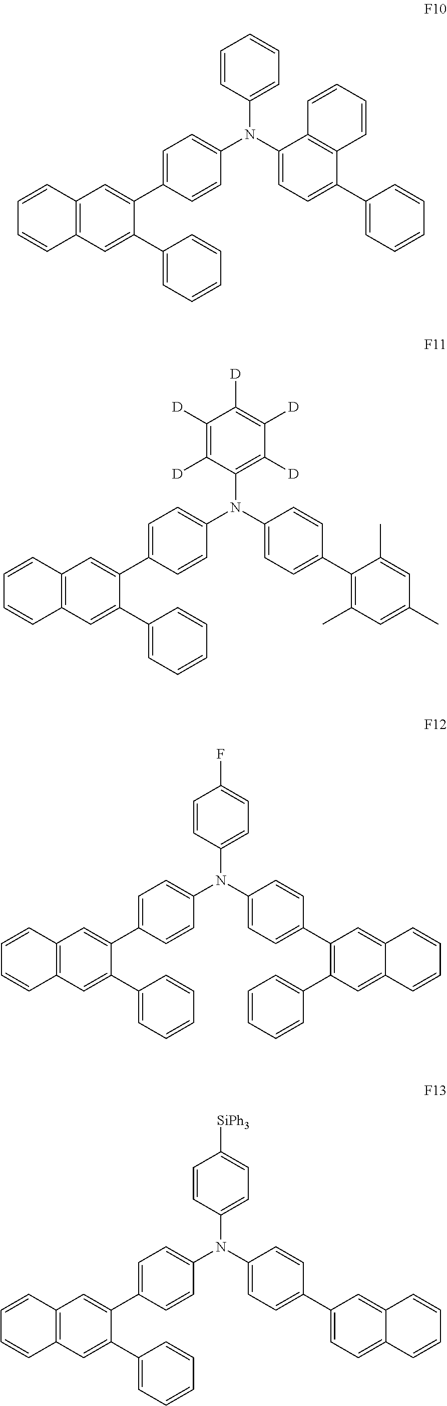

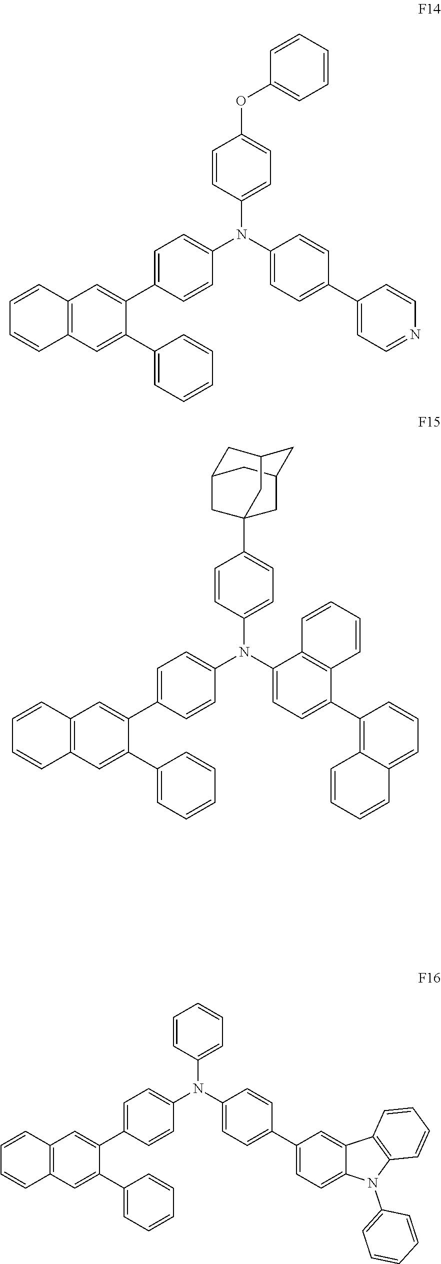 [SMILES: c1ccc(Oc2ccc(N(c3ccc(-c4ccncc4)cc3)c3ccc(-c4cc5ccccc5cc4-c4ccccc4)cc3)cc2)cc1 ; c1ccc(-c2cc3ccccc3cc2-c2ccc(N(c3ccc(C45CC6CC(CC(C6)C4)C5)cc3)c3ccc(-c4cccc5ccccc45)c4ccccc34)cc2)cc1 ; c1ccc(-c2cc3ccccc3cc2-c2ccc(N(c3ccccc3)c3ccc(-c4ccc5c(c4)c4ccccc4n5-c4ccccc4)cc3)cc2)cc1]

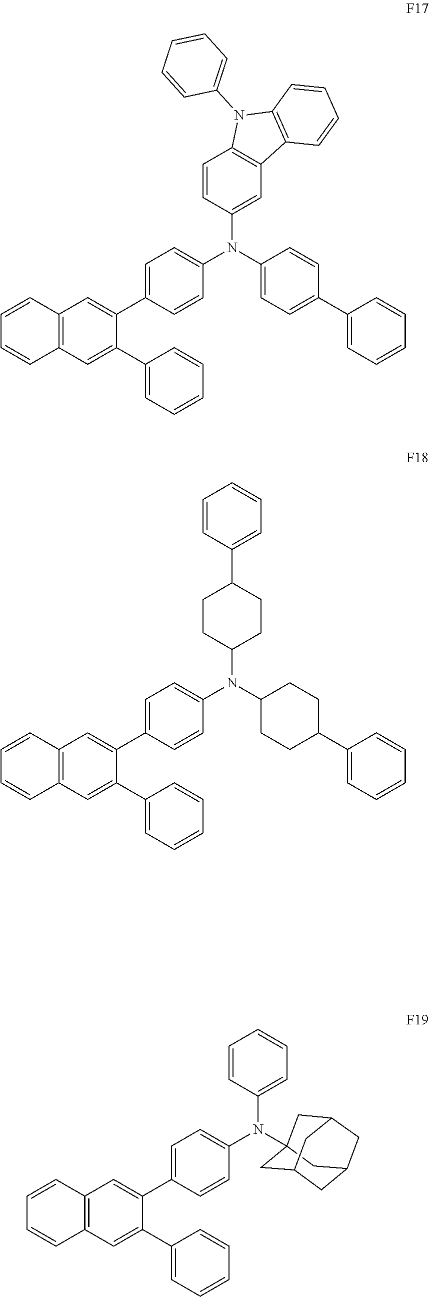 [SMILES: c1ccc(-c2ccc(N(c3ccc(-c4cc5ccccc5cc4-c4ccccc4)cc3)c3ccc4c(c3)c3ccccc3n4-c3ccccc3)cc2)cc1 ; c1ccc(-c2cc3ccccc3cc2-c2ccc(N(C3CCC(c4ccccc4)CC3)C3CCC(c4ccccc4)CC3)cc2)cc1 ; c1ccc(-c2cc3ccccc3cc2-c2ccc(N(c3ccccc3)C34CC5CC(CC(C5)C3)C4)cc2)cc1]

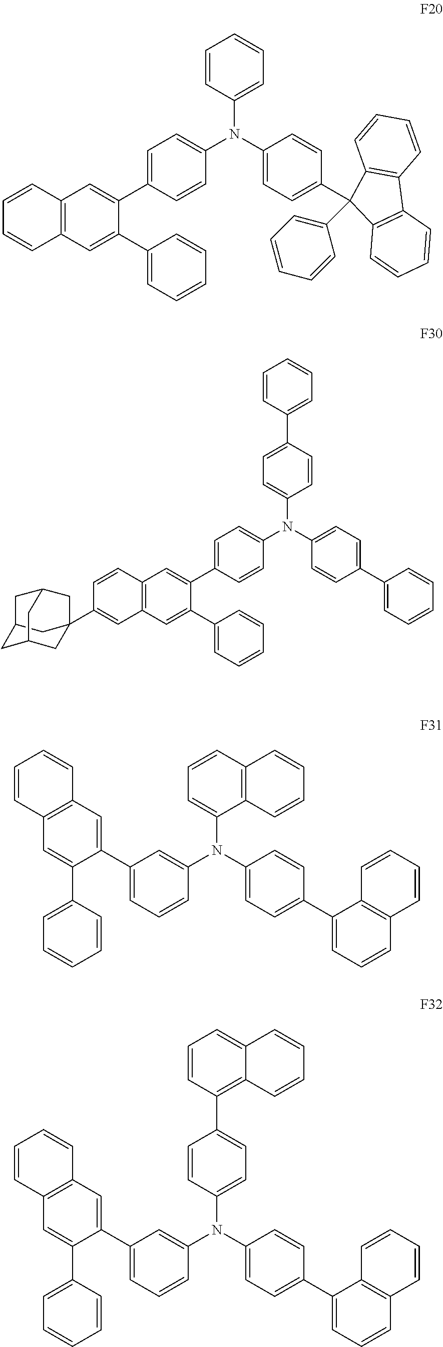 [SMILES: c1ccc(-c2cc3ccccc3cc2-c2ccc(N(c3ccccc3)c3ccc(C4(c5ccccc5)c5ccccc5-c5ccccc54)cc3)cc2)cc1 ; c1ccc(-c2ccc(N(c3ccc(-c4ccccc4)cc3)c3ccc(-c4cc5ccc(C67CC8CC(CC(C8)C6)C7)cc5cc4-c4ccccc4)cc3)cc2)cc1 ; c1ccc(-c2cc3ccccc3cc2-c2cccc(N(c3ccc(-c4cccc5ccccc45)cc3)c3cccc4ccccc34)c2)cc1 ; c1ccc(-c2cc3ccccc3cc2-c2cccc(N(c3ccc(-c4cccc5ccccc45)cc3)c3ccc(-c4cccc5ccccc45)cc3)c2)cc1]

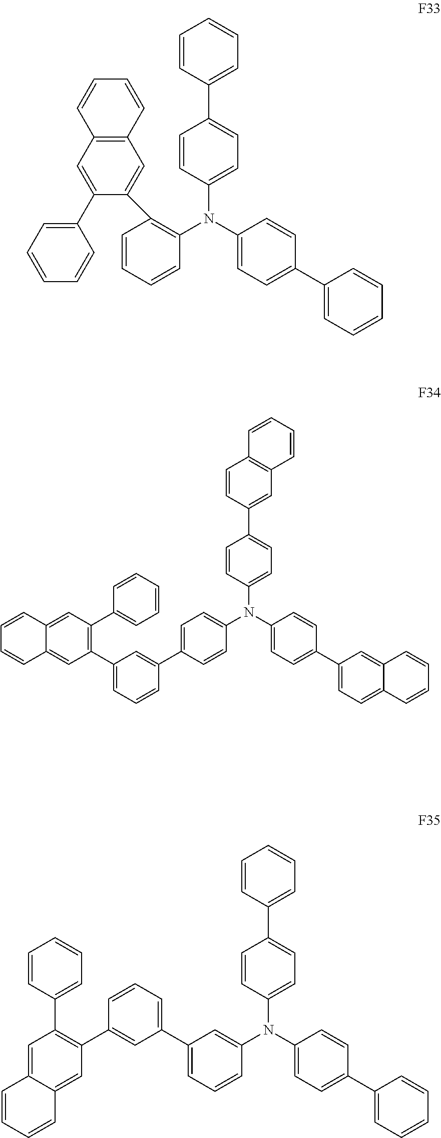 [SMILES: c1ccc(-c2ccc(N(c3ccc(-c4ccccc4)cc3)c3ccccc3-c3cc4ccccc4cc3-c3ccccc3)cc2)cc1 ; c1ccc(-c2cc3ccccc3cc2-c2cccc(-c3ccc(N(c4ccc(-c5ccc6ccccc6c5)cc4)c4ccc(-c5ccc6ccccc6c5)cc4)cc3)c2)cc1 ; c1ccc(-c2ccc(N(c3ccc(-c4ccccc4)cc3)c3cccc(-c4cccc(-c5cc6ccccc6cc5-c5ccccc5)c4)c3)cc2)cc1]

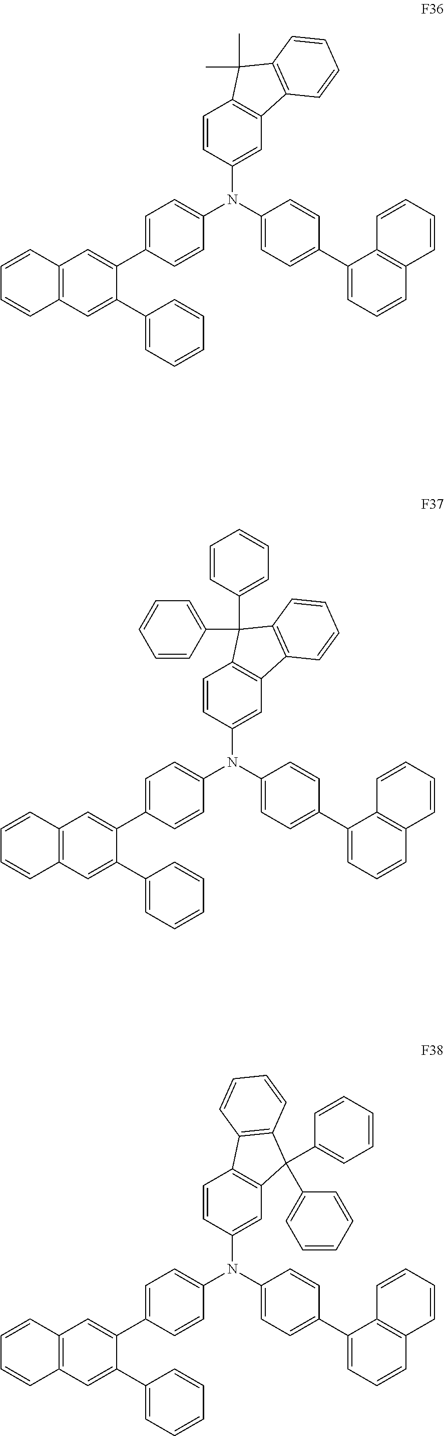 [SMILES: CC1(C)c2ccccc2-c2cc(N(c3ccc(-c4cc5ccccc5cc4-c4ccccc4)cc3)c3ccc(-c4cccc5ccccc45)cc3)ccc21 ; c1ccc(-c2cc3ccccc3cc2-c2ccc(N(c3ccc(-c4cccc5ccccc45)cc3)c3ccc4c(c3)-c3ccccc3C4(c3ccccc3)c3ccccc3)cc2)cc1 ; c1ccc(-c2cc3ccccc3cc2-c2ccc(N(c3ccc(-c4cccc5ccccc45)cc3)c3ccc4c(c3)C(c3ccccc3)(c3ccccc3)c3ccccc3-4)cc2)cc1]

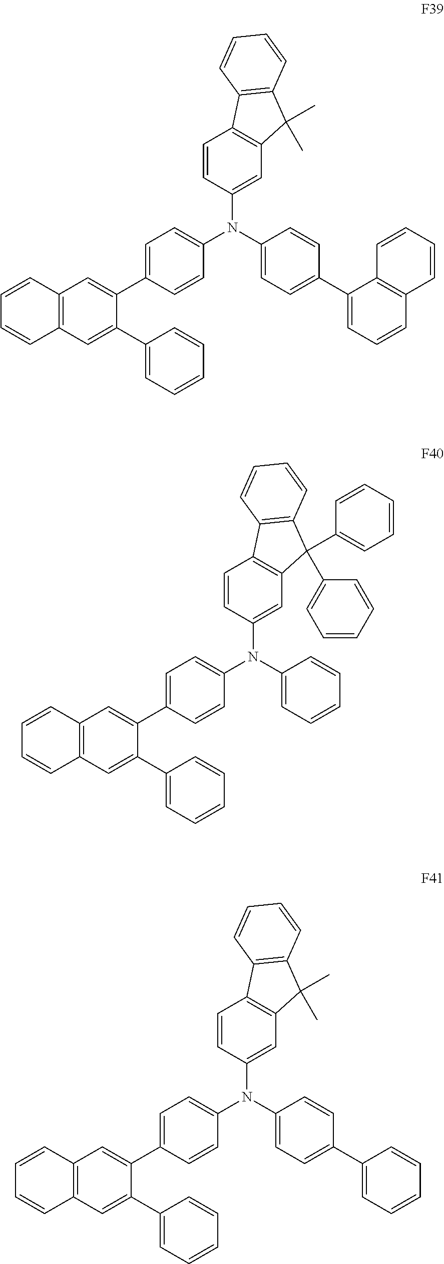 [SMILES: CC1(C)c2ccccc2-c2ccc(N(c3ccc(-c4cc5ccccc5cc4-c4ccccc4)cc3)c3ccc(-c4cccc5ccccc45)cc3)cc21 ; c1ccc(-c2cc3ccccc3cc2-c2ccc(N(c3ccccc3)c3ccc4c(c3)C(c3ccccc3)(c3ccccc3)c3ccccc3-4)cc2)cc1 ; CC1(C)c2ccccc2-c2ccc(N(c3ccc(-c4ccccc4)cc3)c3ccc(-c4cc5ccccc5cc4-c4ccccc4)cc3)cc21]

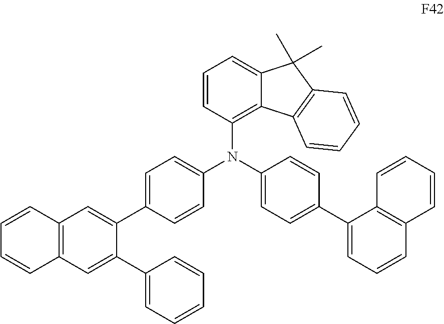 [SMILES: CC1(C)c2ccccc2-c2c(N(c3ccc(-c4cc5ccccc5cc4-c4ccccc4)cc3)c3ccc(-c4cccc5ccccc45)cc3)cccc21]

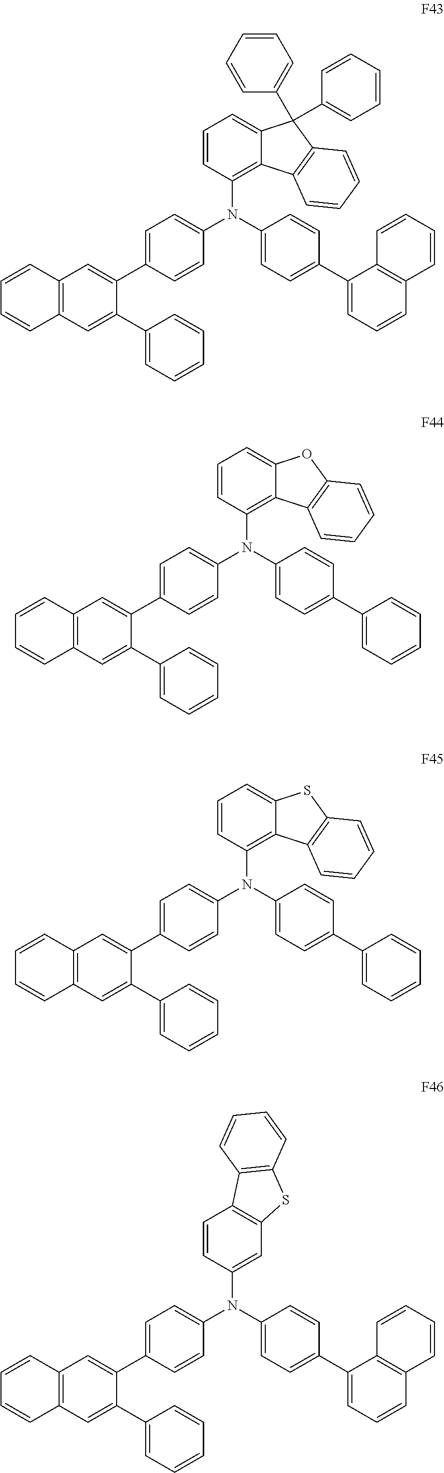 [SMILES: c1ccc(-c2cc3ccccc3cc2-c2ccc(N(c3ccc(-c4cccc5ccccc45)cc3)c3cccc4c3-c3ccccc3C4(c3ccccc3)c3ccccc3)cc2)cc1 ; c1ccc(-c2ccc(N(c3ccc(-c4cc5ccccc5cc4-c4ccccc4)cc3)c3cccc4oc5ccccc5c34)cc2)cc1 ; c1ccc(-c2ccc(N(c3ccc(-c4cc5ccccc5cc4-c4ccccc4)cc3)c3cccc4sc5ccccc5c34)cc2)cc1 ; c1ccc(-c2cc3ccccc3cc2-c2ccc(N(c3ccc(-c4cccc5ccccc45)cc3)c3ccc4c(c3)sc3ccccc34)cc2)cc1]

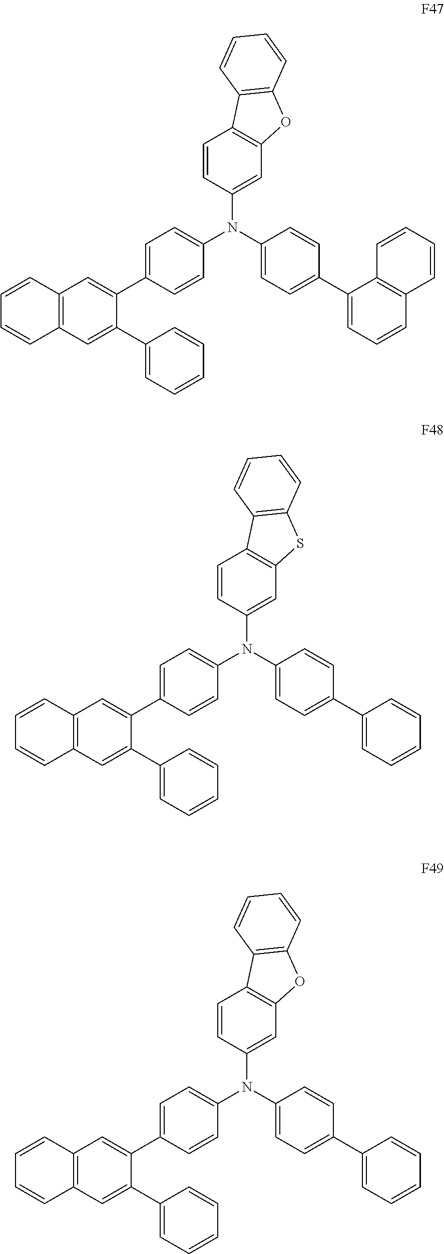 [SMILES: c1ccc(-c2cc3ccccc3cc2-c2ccc(N(c3ccc(-c4cccc5ccccc45)cc3)c3ccc4c(c3)oc3ccccc34)cc2)cc1 ; c1ccc(-c2ccc(N(c3ccc(-c4cc5ccccc5cc4-c4ccccc4)cc3)c3ccc4c(c3)sc3ccccc34)cc2)cc1 ; c1ccc(-c2ccc(N(c3ccc(-c4cc5ccccc5cc4-c4ccccc4)cc3)c3ccc4c(c3)oc3ccccc34)cc2)cc1]

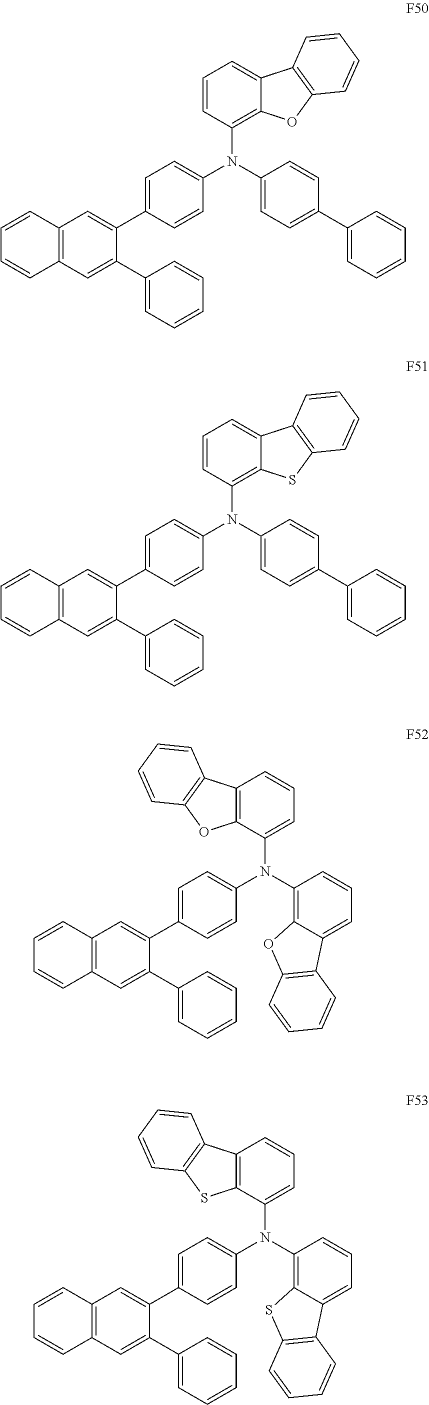 [SMILES: c1ccc(-c2ccc(N(c3ccc(-c4cc5ccccc5cc4-c4ccccc4)cc3)c3cccc4c3oc3ccccc34)cc2)cc1 ; c1ccc(-c2ccc(N(c3ccc(-c4cc5ccccc5cc4-c4ccccc4)cc3)c3cccc4c3sc3ccccc34)cc2)cc1 ; c1ccc(-c2cc3ccccc3cc2-c2ccc(N(c3cccc4c3oc3ccccc34)c3cccc4c3oc3ccccc34)cc2)cc1 ; c1ccc(-c2cc3ccccc3cc2-c2ccc(N(c3cccc4c3sc3ccccc34)c3cccc4c3sc3ccccc34)cc2)cc1]

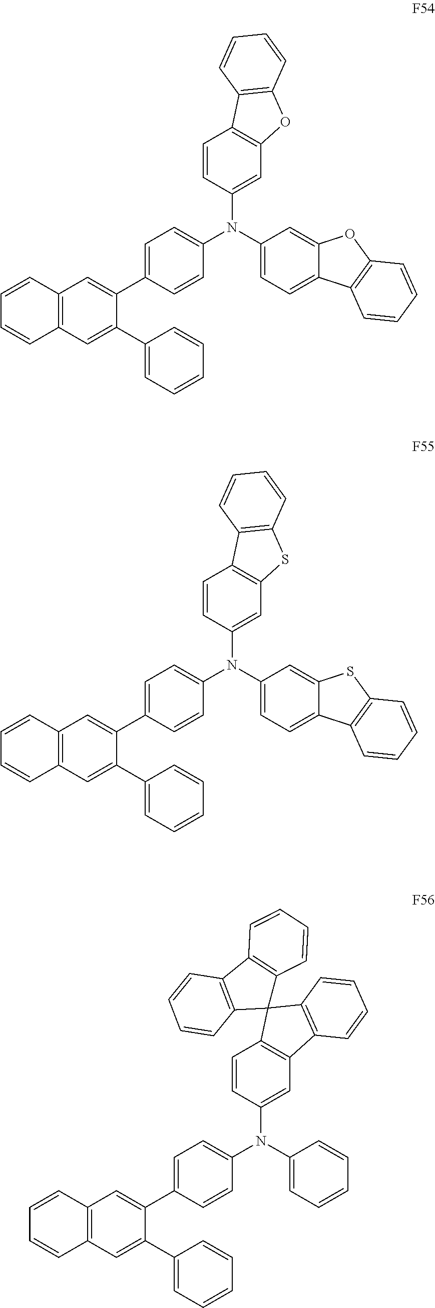 [SMILES: c1ccc(-c2cc3ccccc3cc2-c2ccc(N(c3ccc4c(c3)oc3ccccc34)c3ccc4c(c3)oc3ccccc34)cc2)cc1 ; c1ccc(-c2cc3ccccc3cc2-c2ccc(N(c3ccc4c(c3)sc3ccccc34)c3ccc4c(c3)sc3ccccc34)cc2)cc1 ; c1ccc(-c2cc3ccccc3cc2-c2ccc(N(c3ccccc3)c3ccc4c(c3)-c3ccccc3C43c4ccccc4-c4ccccc43)cc2)cc1]

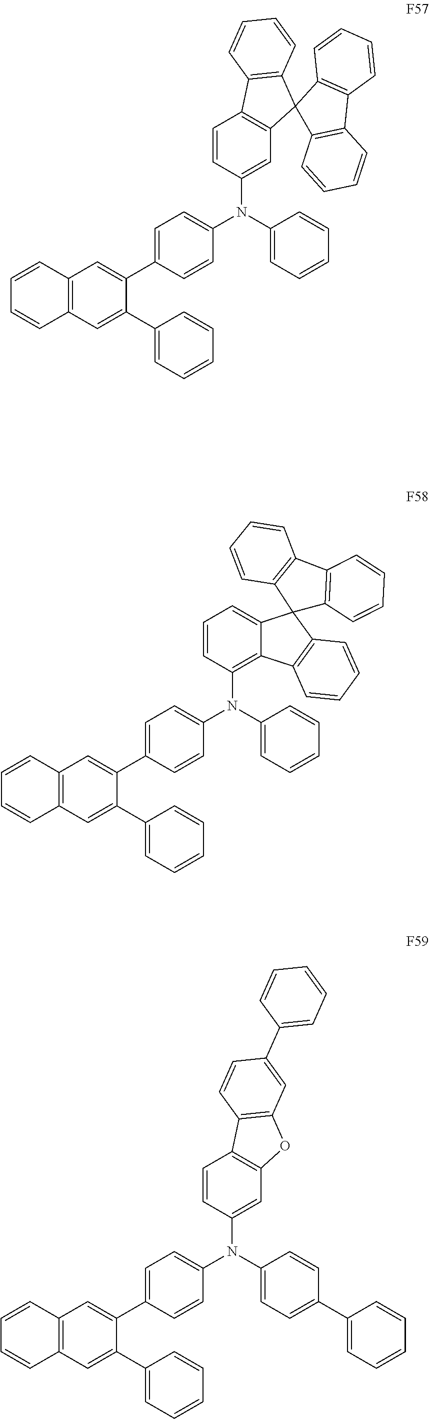 [SMILES: c1ccc(-c2cc3ccccc3cc2-c2ccc(N(c3ccccc3)c3ccc4c(c3)C3(c5ccccc5-c5ccccc53)c3ccccc3-4)cc2)cc1 ; c1ccc(-c2cc3ccccc3cc2-c2ccc(N(c3ccccc3)c3cccc4c3-c3ccccc3C43c4ccccc4-c4ccccc43)cc2)cc1 ; c1ccc(-c2ccc(N(c3ccc(-c4cc5ccccc5cc4-c4ccccc4)cc3)c3ccc4c(c3)oc3cc(-c5ccccc5)ccc34)cc2)cc1]

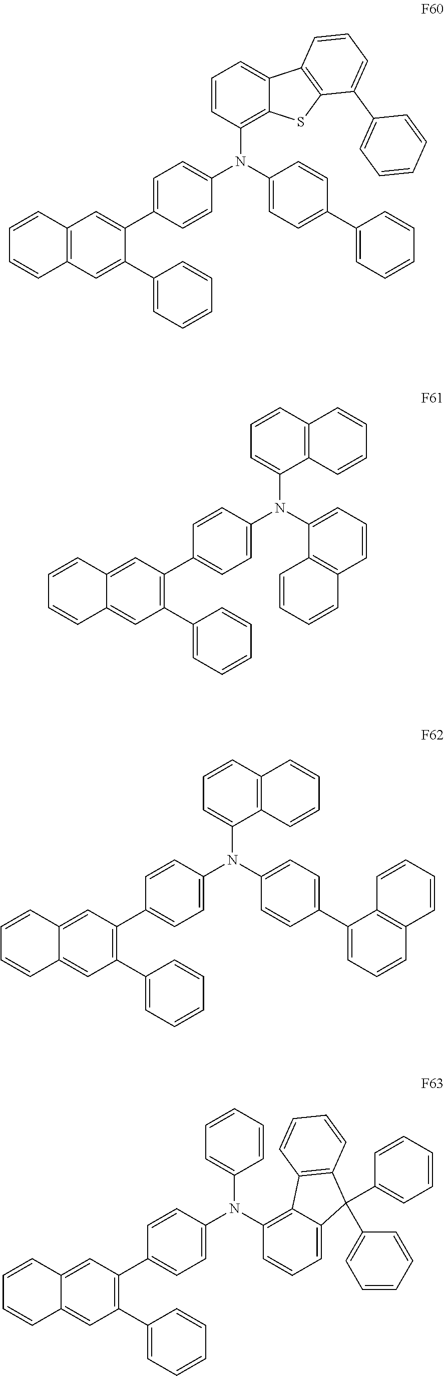 [SMILES: c1ccc(-c2ccc(N(c3ccc(-c4cc5ccccc5cc4-c4ccccc4)cc3)c3cccc4c3sc3c(-c5ccccc5)cccc34)cc2)cc1 ; c1ccc(-c2cc3ccccc3cc2-c2ccc(N(c3cccc4ccccc34)c3cccc4ccccc34)cc2)cc1 ; c1ccc(-c2cc3ccccc3cc2-c2ccc(N(c3ccc(-c4cccc5ccccc45)cc3)c3cccc4ccccc34)cc2)cc1 ; c1ccc(-c2cc3ccccc3cc2-c2ccc(N(c3ccccc3)c3cccc4c3-c3ccccc3C4(c3ccccc3)c3ccccc3)cc2)cc1]

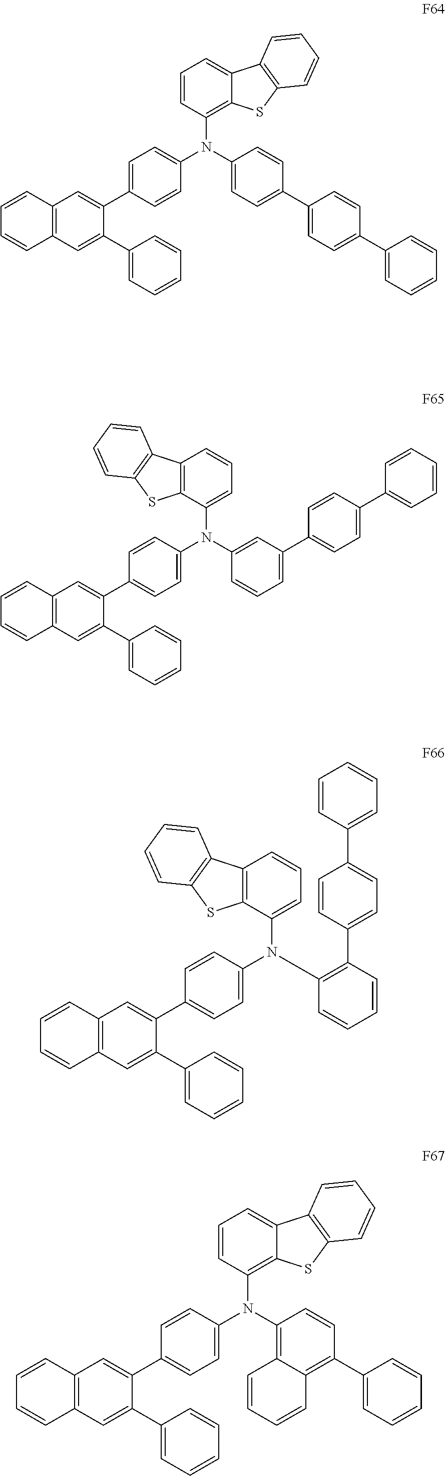 [SMILES: c1ccc(-c2ccc(-c3ccc(N(c4ccc(-c5cc6ccccc6cc5-c5ccccc5)cc4)c4cccc5c4sc4ccccc45)cc3)cc2)cc1 ; c1ccc(-c2ccc(-c3cccc(N(c4ccc(-c5cc6ccccc6cc5-c5ccccc5)cc4)c4cccc5c4sc4ccccc45)c3)cc2)cc1 ; c1ccc(-c2ccc(-c3ccccc3N(c3ccc(-c4cc5ccccc5cc4-c4ccccc4)cc3)c3cccc4c3sc3ccccc34)cc2)cc1 ; c1ccc(-c2cc3ccccc3cc2-c2ccc(N(c3ccc(-c4ccccc4)c4ccccc34)c3cccc4c3sc3ccccc34)cc2)cc1]

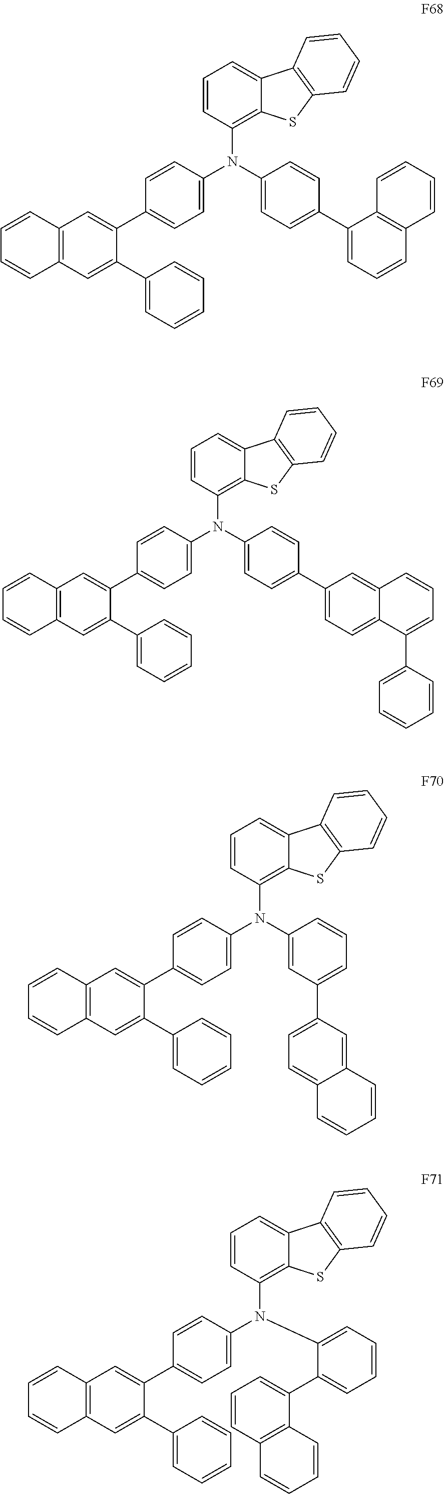 [SMILES: c1ccc(-c2cc3ccccc3cc2-c2ccc(N(c3ccc(-c4cccc5ccccc45)cc3)c3cccc4c3sc3ccccc34)cc2)cc1 ; c1ccc(-c2cc3ccccc3cc2-c2ccc(N(c3ccc(-c4ccc5c(-c6ccccc6)cccc5c4)cc3)c3cccc4c3sc3ccccc34)cc2)cc1 ; c1ccc(-c2cc3ccccc3cc2-c2ccc(N(c3cccc(-c4ccc5ccccc5c4)c3)c3cccc4c3sc3ccccc34)cc2)cc1 ; c1ccc(-c2cc3ccccc3cc2-c2ccc(N(c3ccccc3-c3cccc4ccccc34)c3cccc4c3sc3ccccc34)cc2)cc1]

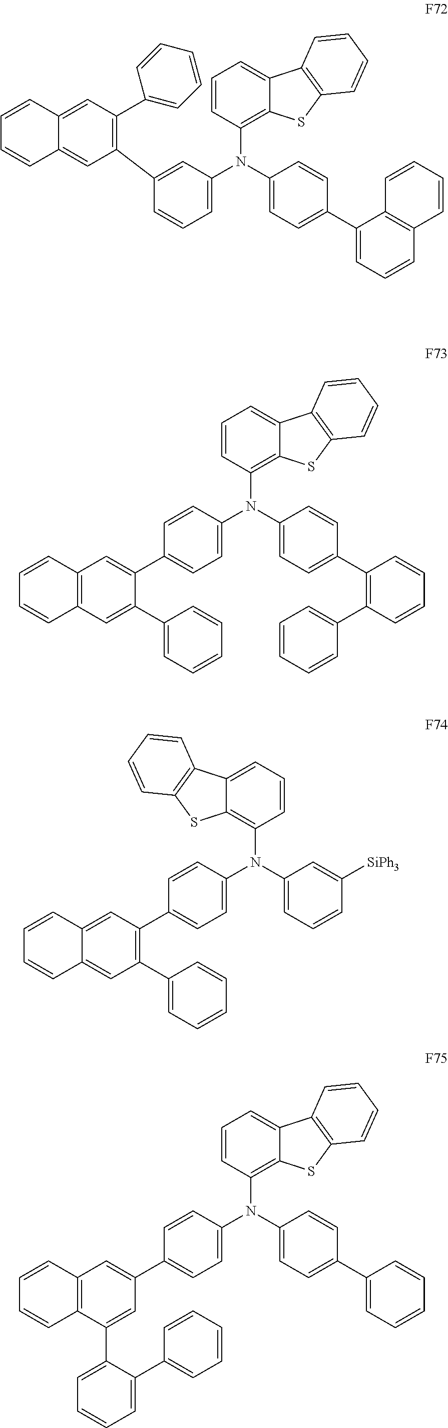

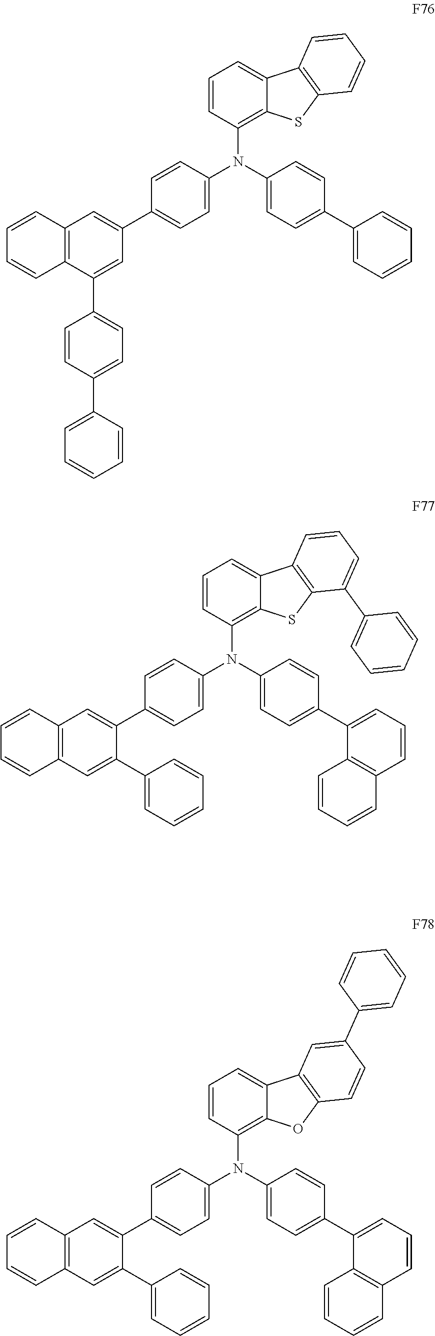 [SMILES: c1ccc(-c2ccc(-c3cc(-c4ccc(N(c5ccc(-c6ccccc6)cc5)c5cccc6c5sc5ccccc56)cc4)cc4ccccc34)cc2)cc1 ; c1ccc(-c2cc3ccccc3cc2-c2ccc(N(c3ccc(-c4cccc5ccccc45)cc3)c3cccc4c3sc3c(-c5ccccc5)cccc34)cc2)cc1 ; c1ccc(-c2ccc3oc4c(N(c5ccc(-c6cc7ccccc7cc6-c6ccccc6)cc5)c5ccc(-c6cccc7ccccc67)cc5)cccc4c3c2)cc1]

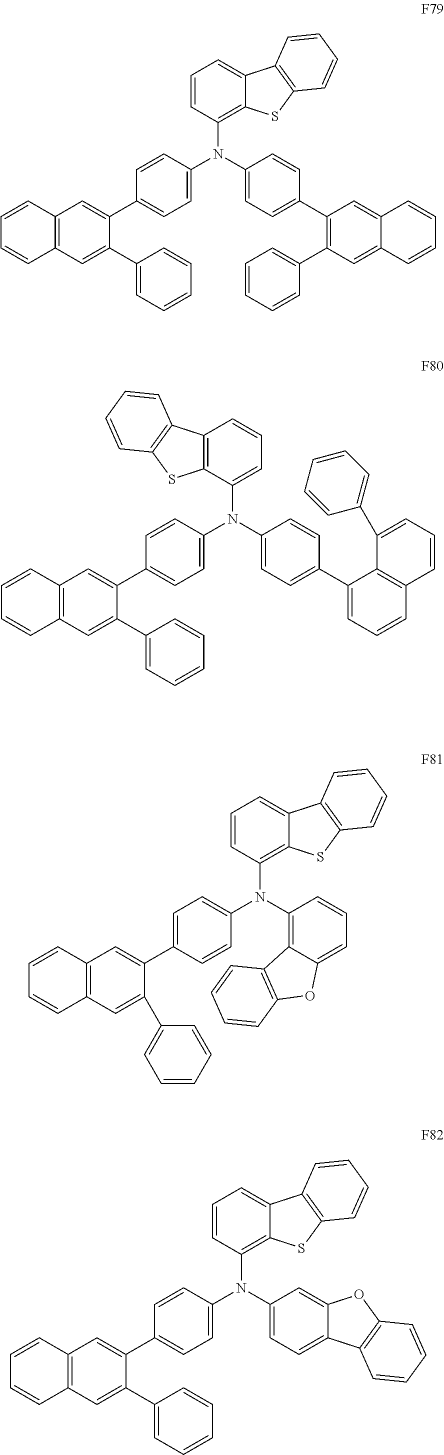 [SMILES: c1ccc(-c2cc3ccccc3cc2-c2ccc(N(c3ccc(-c4cc5ccccc5cc4-c4ccccc4)cc3)c3cccc4c3sc3ccccc34)cc2)cc1 ; c1ccc(-c2cc3ccccc3cc2-c2ccc(N(c3ccc(-c4cccc5cccc(-c6ccccc6)c45)cc3)c3cccc4c3sc3ccccc34)cc2)cc1 ; c1ccc(-c2cc3ccccc3cc2-c2ccc(N(c3cccc4c3sc3ccccc34)c3cccc4oc5ccccc5c34)cc2)cc1 ; c1ccc(-c2cc3ccccc3cc2-c2ccc(N(c3ccc4c(c3)oc3ccccc34)c3cccc4c3sc3ccccc34)cc2)cc1]

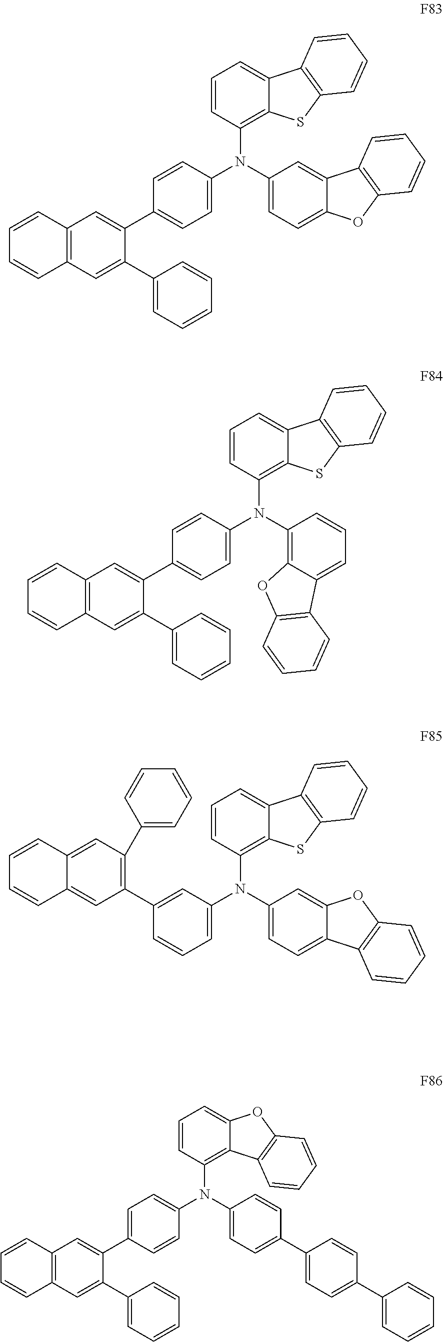 [SMILES: c1ccc(-c2cc3ccccc3cc2-c2ccc(N(c3ccc4oc5ccccc5c4c3)c3cccc4c3sc3ccccc34)cc2)cc1 ; c1ccc(-c2cc3ccccc3cc2-c2ccc(N(c3cccc4c3oc3ccccc34)c3cccc4c3sc3ccccc34)cc2)cc1 ; c1ccc(-c2cc3ccccc3cc2-c2cccc(N(c3ccc4c(c3)oc3ccccc34)c3cccc4c3sc3ccccc34)c2)cc1 ; c1ccc(-c2ccc(-c3ccc(N(c4ccc(-c5cc6ccccc6cc5-c5ccccc5)cc4)c4cccc5oc6ccccc6c45)cc3)cc2)cc1]

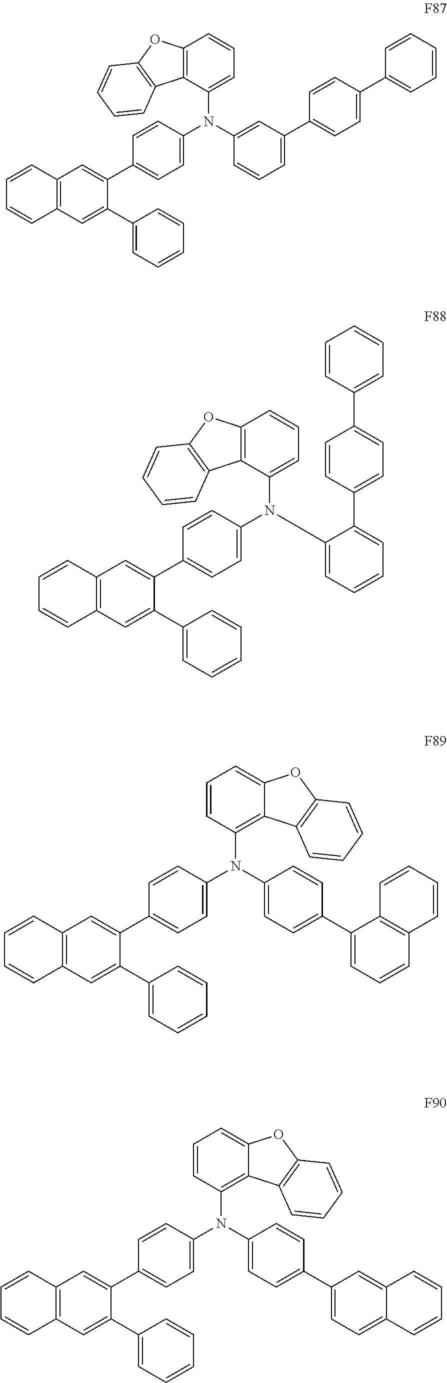 [SMILES: c1ccc(-c2ccc(-c3cccc(N(c4ccc(-c5cc6ccccc6cc5-c5ccccc5)cc4)c4cccc5oc6ccccc6c45)c3)cc2)cc1 ; c1ccc(-c2ccc(-c3ccccc3N(c3ccc(-c4cc5ccccc5cc4-c4ccccc4)cc3)c3cccc4oc5ccccc5c34)cc2)cc1 ; c1ccc(-c2cc3ccccc3cc2-c2ccc(N(c3ccc(-c4cccc5ccccc45)cc3)c3cccc4oc5ccccc5c34)cc2)cc1 ; c1ccc(-c2cc3ccccc3cc2-c2ccc(N(c3ccc(-c4ccc5ccccc5c4)cc3)c3cccc4oc5ccccc5c34)cc2)cc1]

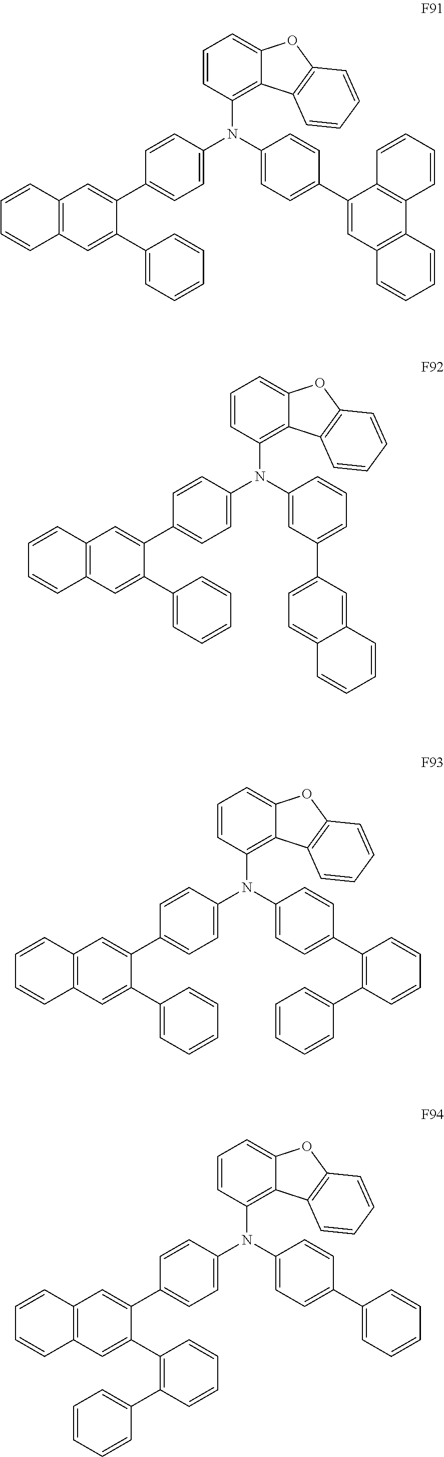 [SMILES: c1ccc(-c2cc3ccccc3cc2-c2ccc(N(c3ccc(-c4cc5ccccc5c5ccccc45)cc3)c3cccc4oc5ccccc5c34)cc2)cc1 ; c1ccc(-c2cc3ccccc3cc2-c2ccc(N(c3cccc(-c4ccc5ccccc5c4)c3)c3cccc4oc5ccccc5c34)cc2)cc1 ; c1ccc(-c2ccccc2-c2ccc(N(c3ccc(-c4cc5ccccc5cc4-c4ccccc4)cc3)c3cccc4oc5ccccc5c34)cc2)cc1 ; c1ccc(-c2ccc(N(c3ccc(-c4cc5ccccc5cc4-c4ccccc4-c4ccccc4)cc3)c3cccc4oc5ccccc5c34)cc2)cc1]

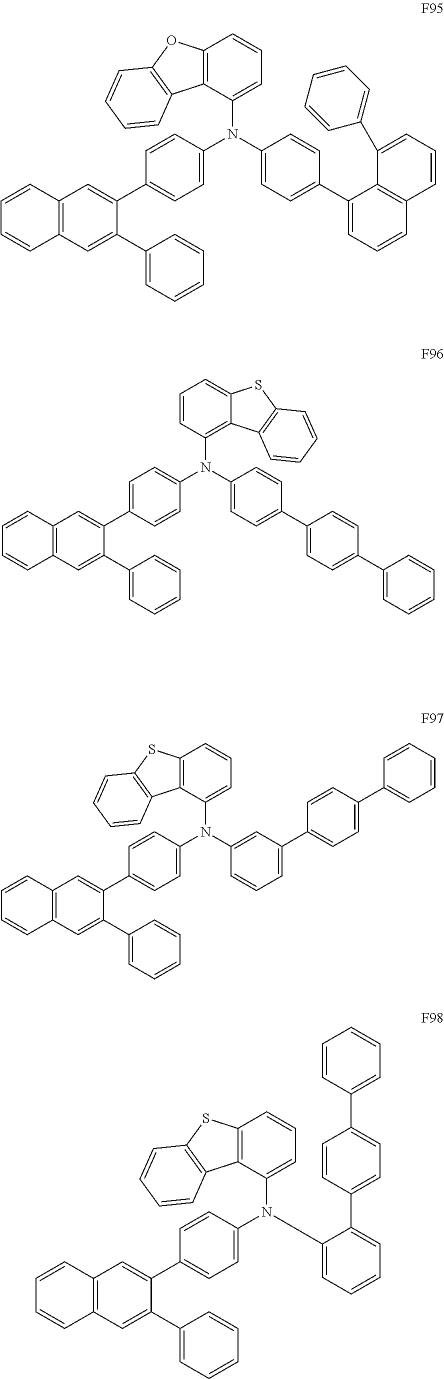 [SMILES: c1ccc(-c2cc3ccccc3cc2-c2ccc(N(c3ccc(-c4cccc5cccc(-c6ccccc6)c45)cc3)c3cccc4oc5ccccc5c34)cc2)cc1 ; c1ccc(-c2ccc(-c3ccc(N(c4ccc(-c5cc6ccccc6cc5-c5ccccc5)cc4)c4cccc5sc6ccccc6c45)cc3)cc2)cc1 ; c1ccc(-c2ccc(-c3cccc(N(c4ccc(-c5cc6ccccc6cc5-c5ccccc5)cc4)c4cccc5sc6ccccc6c45)c3)cc2)cc1 ; c1ccc(-c2ccc(-c3ccccc3N(c3ccc(-c4cc5ccccc5cc4-c4ccccc4)cc3)c3cccc4sc5ccccc5c34)cc2)cc1]

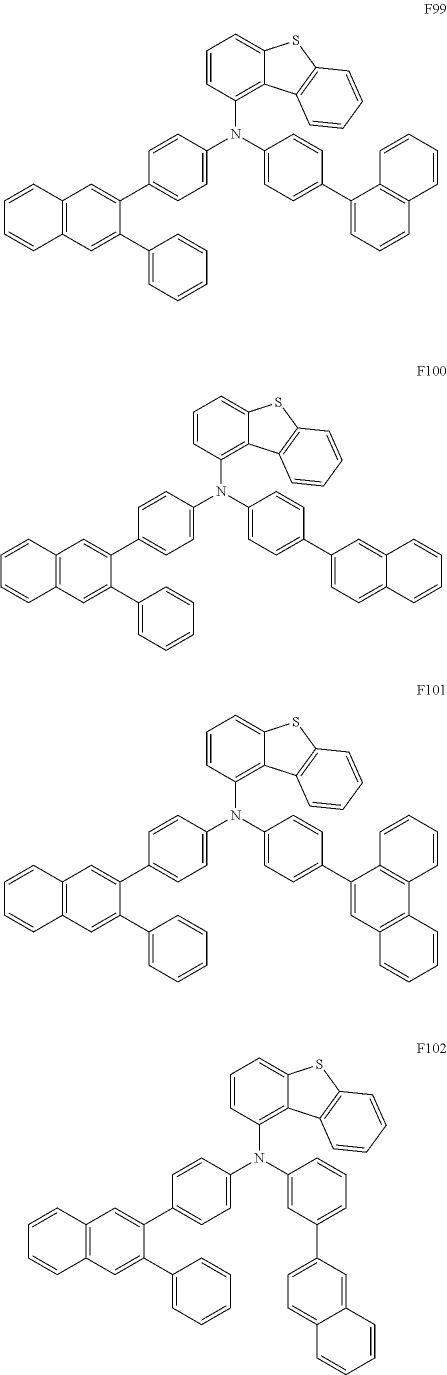 [SMILES: c1ccc(-c2cc3ccccc3cc2-c2ccc(N(c3ccc(-c4cccc5ccccc45)cc3)c3cccc4sc5ccccc5c34)cc2)cc1 ; c1ccc(-c2cc3ccccc3cc2-c2ccc(N(c3ccc(-c4ccc5ccccc5c4)cc3)c3cccc4sc5ccccc5c34)cc2)cc1 ; c1ccc(-c2cc3ccccc3cc2-c2ccc(N(c3ccc(-c4cc5ccccc5c5ccccc45)cc3)c3cccc4sc5ccccc5c34)cc2)cc1 ; c1ccc(-c2cc3ccccc3cc2-c2ccc(N(c3cccc(-c4ccc5ccccc5c4)c3)c3cccc4sc5ccccc5c34)cc2)cc1]

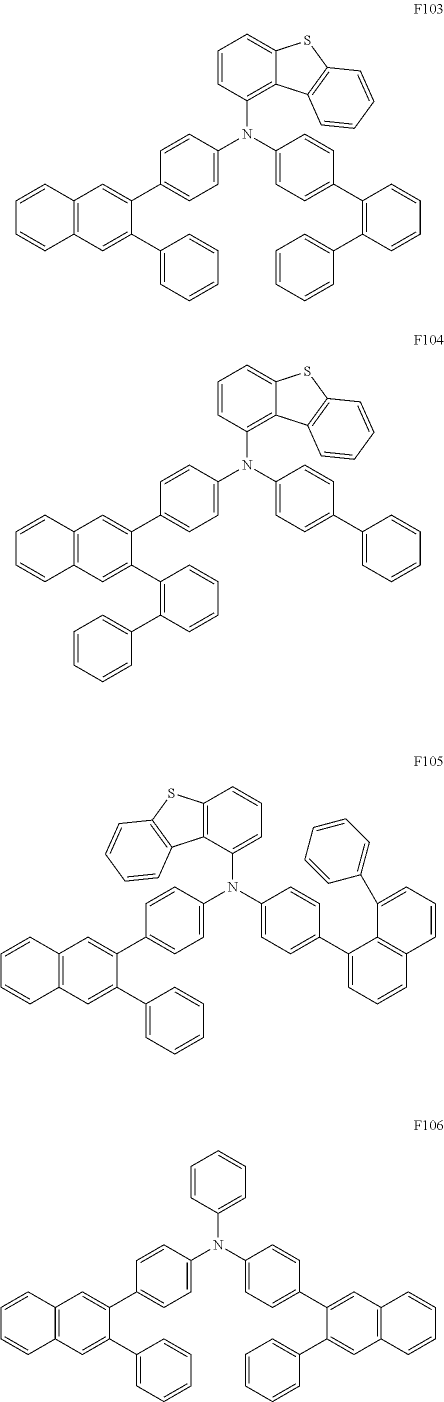 [SMILES: c1ccc(-c2ccccc2-c2ccc(N(c3ccc(-c4cc5ccccc5cc4-c4ccccc4)cc3)c3cccc4sc5ccccc5c34)cc2)cc1 ; c1ccc(-c2ccc(N(c3ccc(-c4cc5ccccc5cc4-c4ccccc4-c4ccccc4)cc3)c3cccc4sc5ccccc5c34)cc2)cc1 ; c1ccc(-c2cc3ccccc3cc2-c2ccc(N(c3ccc(-c4cccc5cccc(-c6ccccc6)c45)cc3)c3cccc4sc5ccccc5c34)cc2)cc1 ; c1ccc(-c2cc3ccccc3cc2-c2ccc(N(c3ccccc3)c3ccc(-c4cc5ccccc5cc4-c4ccccc4)cc3)cc2)cc1]

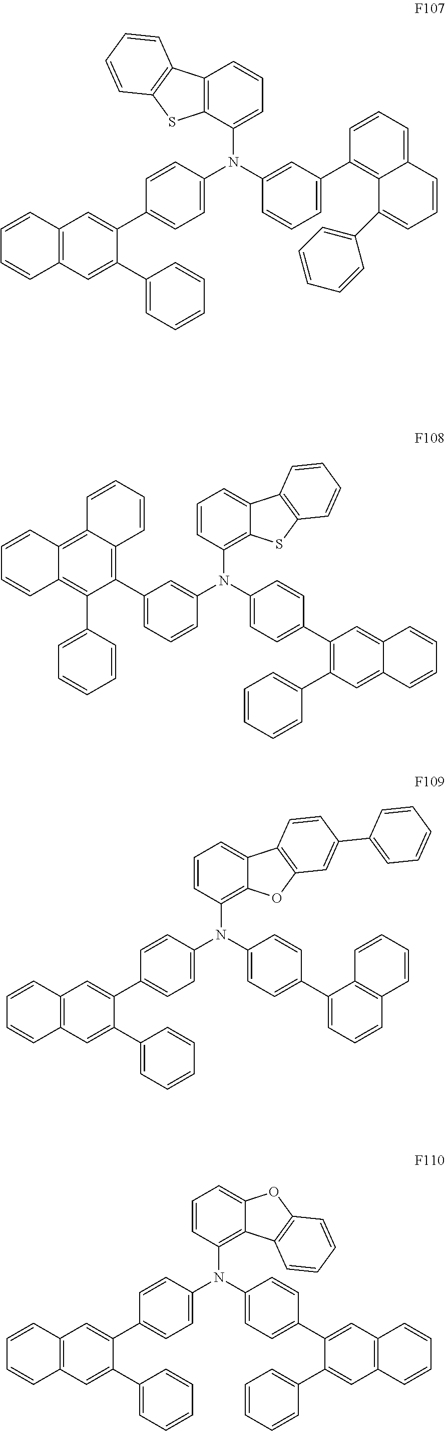 [SMILES: c1ccc(-c2cc3ccccc3cc2-c2ccc(N(c3cccc(-c4cccc5cccc(-c6ccccc6)c45)c3)c3cccc4c3sc3ccccc34)cc2)cc1 ; c1ccc(-c2cc3ccccc3cc2-c2ccc(N(c3cccc(-c4c(-c5ccccc5)c5ccccc5c5ccccc45)c3)c3cccc4c3sc3ccccc34)cc2)cc1 ; c1ccc(-c2ccc3c(c2)oc2c(N(c4ccc(-c5cc6ccccc6cc5-c5ccccc5)cc4)c4ccc(-c5cccc6ccccc56)cc4)cccc23)cc1 ; c1ccc(-c2cc3ccccc3cc2-c2ccc(N(c3ccc(-c4cc5ccccc5cc4-c4ccccc4)cc3)c3cccc4oc5ccccc5c34)cc2)cc1]

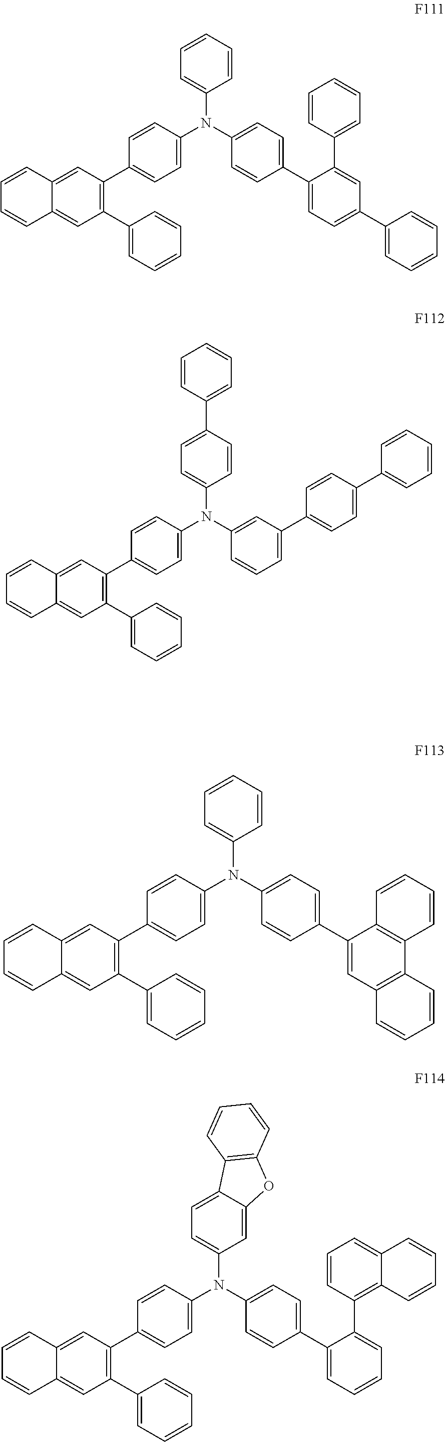 [SMILES: c1ccc(-c2ccc(-c3ccc(N(c4ccccc4)c4ccc(-c5cc6ccccc6cc5-c5ccccc5)cc4)cc3)c(-c3ccccc3)c2)cc1 ; c1ccc(-c2ccc(-c3cccc(N(c4ccc(-c5ccccc5)cc4)c4ccc(-c5cc6ccccc6cc5-c5ccccc5)cc4)c3)cc2)cc1 ; c1ccc(-c2cc3ccccc3cc2-c2ccc(N(c3ccccc3)c3ccc(-c4cc5ccccc5c5ccccc45)cc3)cc2)cc1 ; c1ccc(-c2cc3ccccc3cc2-c2ccc(N(c3ccc(-c4ccccc4-c4cccc5ccccc45)cc3)c3ccc4c(c3)oc3ccccc34)cc2)cc1]

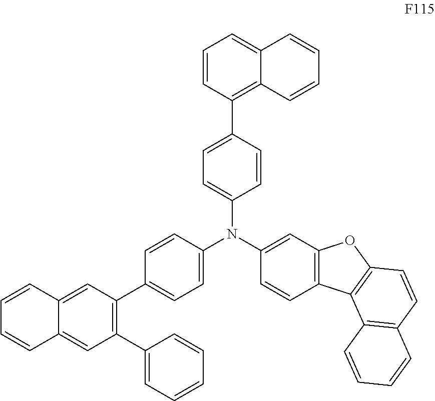 [SMILES: c1ccc(-c2cc3ccccc3cc2-c2ccc(N(c3ccc(-c4cccc5ccccc45)cc3)c3ccc4c(c3)oc3ccc5ccccc5c34)cc2)cc1]

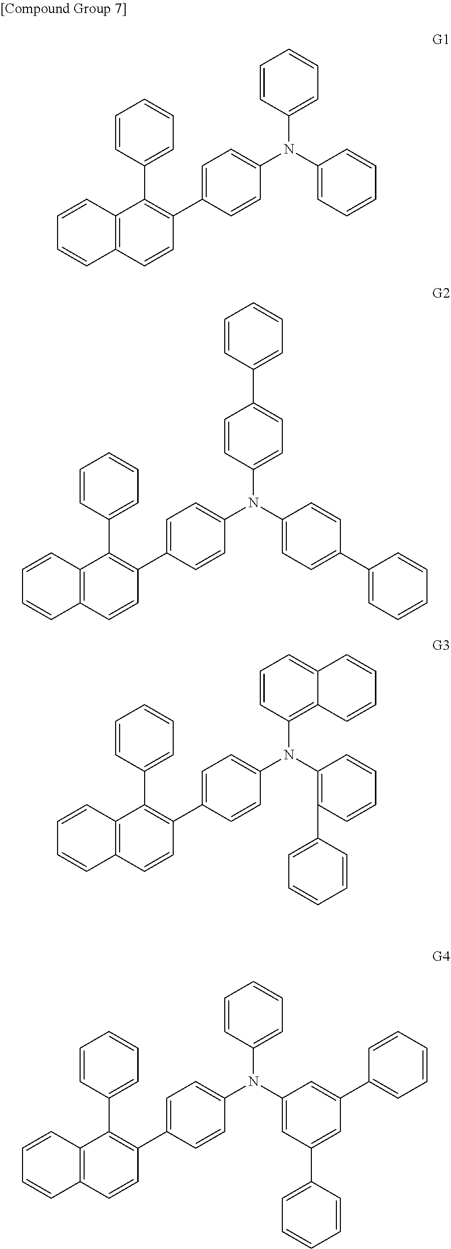 [SMILES: c1ccc(-c2c(-c3ccc(N(c4ccccc4)c4ccccc4)cc3)ccc3ccccc23)cc1 ; c1ccc(-c2ccc(N(c3ccc(-c4ccccc4)cc3)c3ccc(-c4ccc5ccccc5c4-c4ccccc4)cc3)cc2)cc1 ; c1ccc(-c2ccccc2N(c2ccc(-c3ccc4ccccc4c3-c3ccccc3)cc2)c2cccc3ccccc23)cc1 ; c1ccc(-c2cc(-c3ccccc3)cc(N(c3ccccc3)c3ccc(-c4ccc5ccccc5c4-c4ccccc4)cc3)c2)cc1]

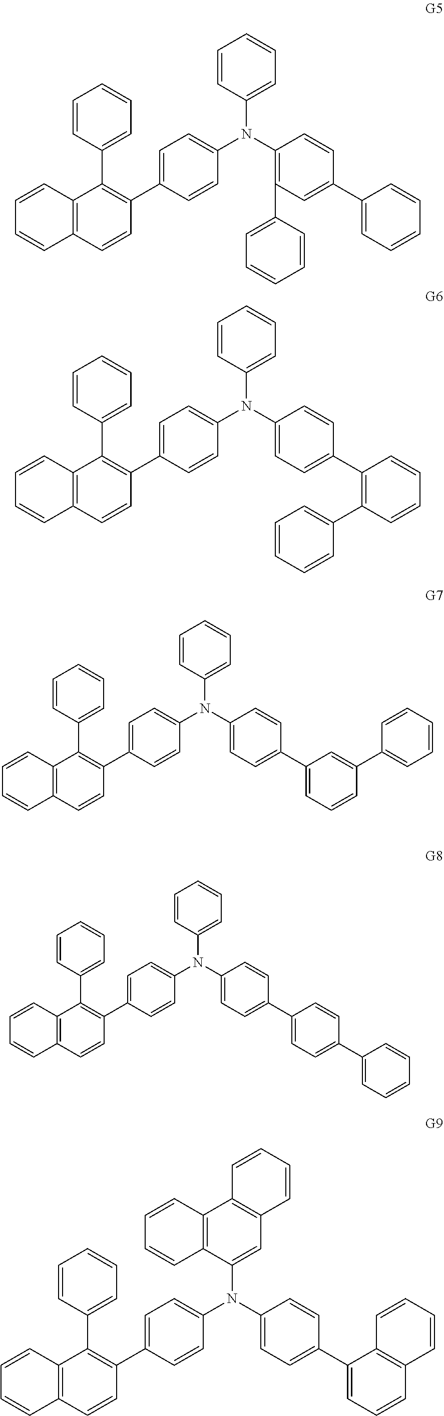

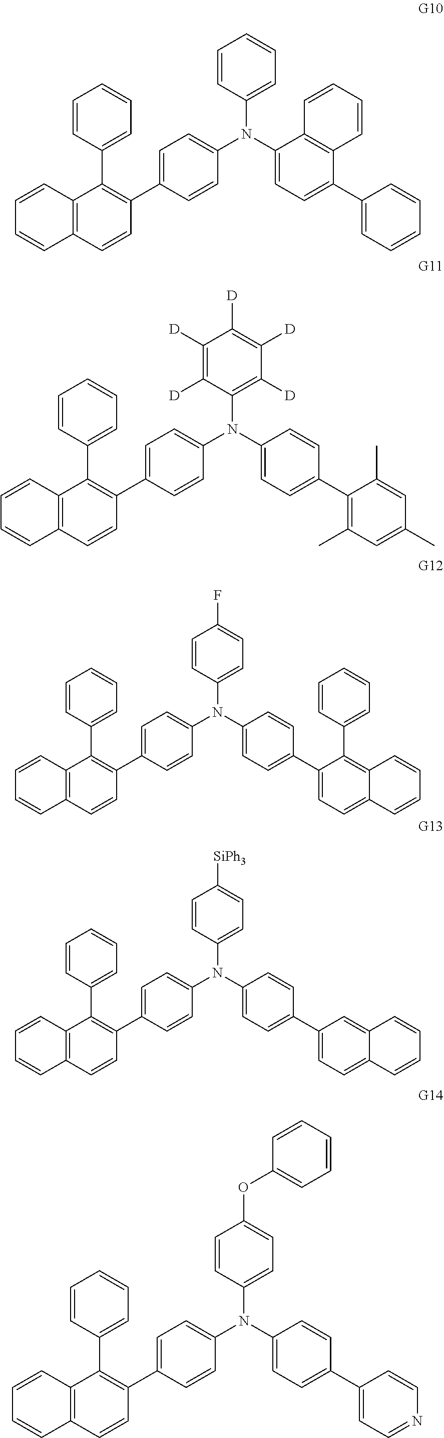

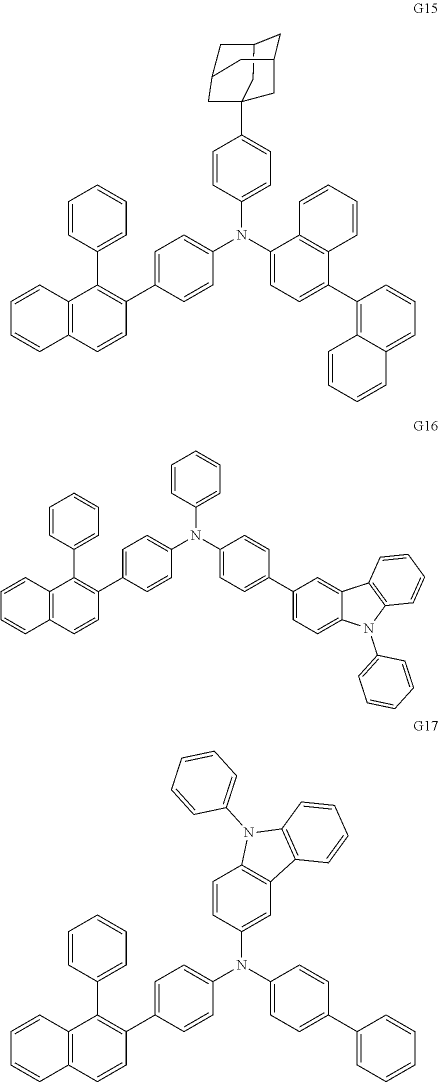 [SMILES: c1ccc(-c2c(-c3ccc(N(c4ccc(C56CC7CC(CC(C7)C5)C6)cc4)c4ccc(-c5cccc6ccccc56)c5ccccc45)cc3)ccc3ccccc23)cc1 ; c1ccc(-c2c(-c3ccc(N(c4ccccc4)c4ccc(-c5ccc6c(c5)c5ccccc5n6-c5ccccc5)cc4)cc3)ccc3ccccc23)cc1 ; c1ccc(-c2ccc(N(c3ccc(-c4ccc5ccccc5c4-c4ccccc4)cc3)c3ccc4c(c3)c3ccccc3n4-c3ccccc3)cc2)cc1]

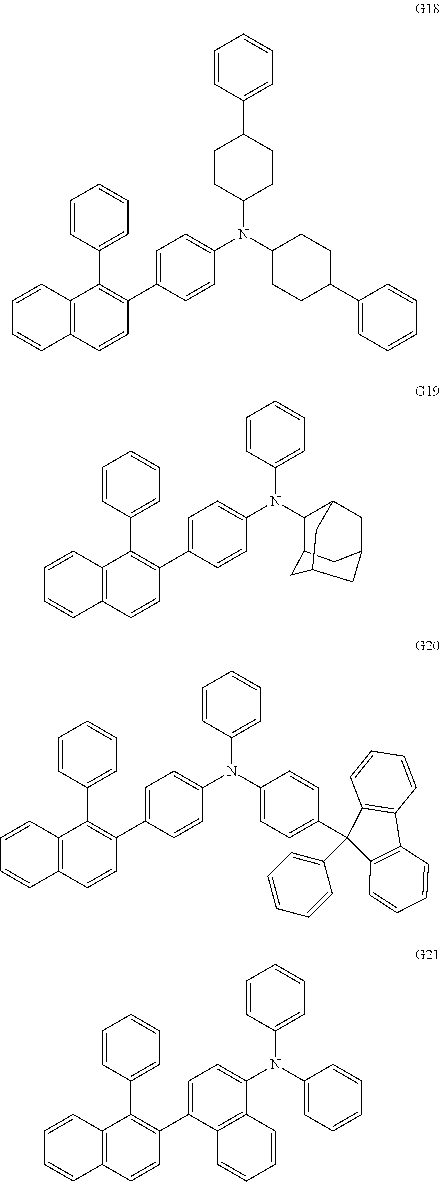 [SMILES: c1ccc(-c2c(-c3ccc(N(C4CCC(c5ccccc5)CC4)C4CCC(c5ccccc5)CC4)cc3)ccc3ccccc23)cc1 ; c1ccc(-c2c(-c3ccc(N(c4ccccc4)C4C5CC6CC(C5)CC4C6)cc3)ccc3ccccc23)cc1 ; c1ccc(-c2c(-c3ccc(N(c4ccccc4)c4ccc(C5(c6ccccc6)c6ccccc6-c6ccccc65)cc4)cc3)ccc3ccccc23)cc1 ; c1ccc(-c2c(-c3ccc(N(c4ccccc4)c4ccccc4)c4ccccc34)ccc3ccccc23)cc1]

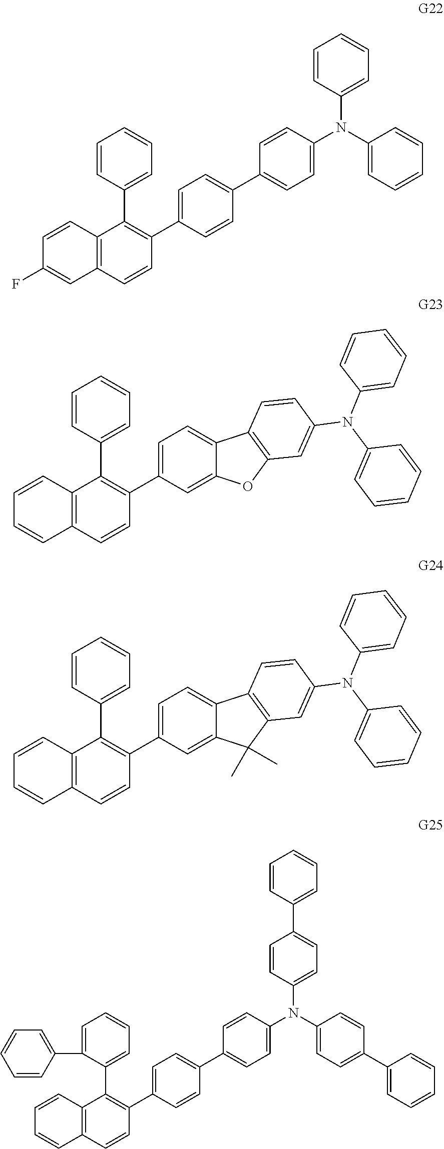 [SMILES: Fc1ccc2c(-c3ccccc3)c(-c3ccc(-c4ccc(N(c5ccccc5)c5ccccc5)cc4)cc3)ccc2c1 ; c1ccc(-c2c(-c3ccc4c(c3)oc3cc(N(c5ccccc5)c5ccccc5)ccc34)ccc3ccccc23)cc1 ; CC1(C)c2cc(-c3ccc4ccccc4c3-c3ccccc3)ccc2-c2ccc(N(c3ccccc3)c3ccccc3)cc21 ; c1ccc(-c2ccc(N(c3ccc(-c4ccccc4)cc3)c3ccc(-c4ccc(-c5ccc6ccccc6c5-c5ccccc5-c5ccccc5)cc4)cc3)cc2)cc1]

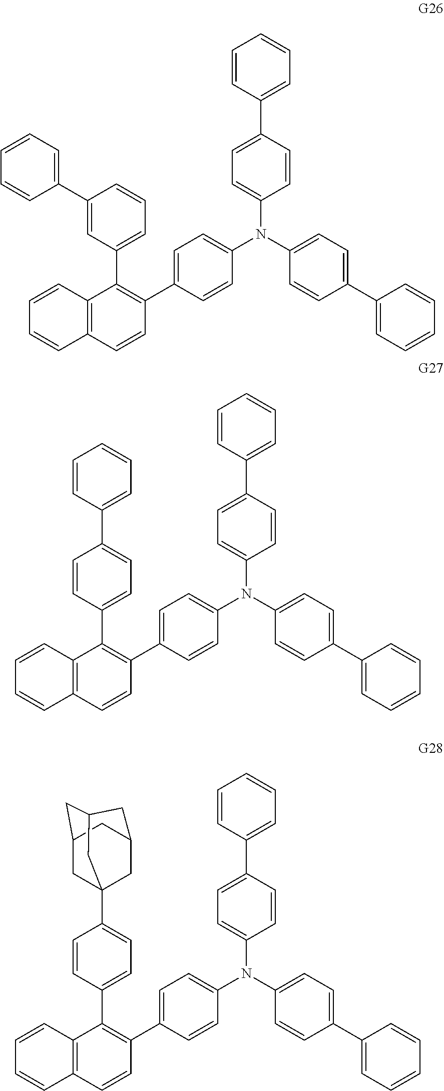 [SMILES: c1ccc(-c2ccc(N(c3ccc(-c4ccccc4)cc3)c3ccc(-c4ccc5ccccc5c4-c4cccc(-c5ccccc5)c4)cc3)cc2)cc1 ; c1ccc(-c2ccc(-c3c(-c4ccc(N(c5ccc(-c6ccccc6)cc5)c5ccc(-c6ccccc6)cc5)cc4)ccc4ccccc34)cc2)cc1 ; c1ccc(-c2ccc(N(c3ccc(-c4ccccc4)cc3)c3ccc(-c4ccc5ccccc5c4-c4ccc(C56CC7CC(CC(C7)C5)C6)cc4)cc3)cc2)cc1]

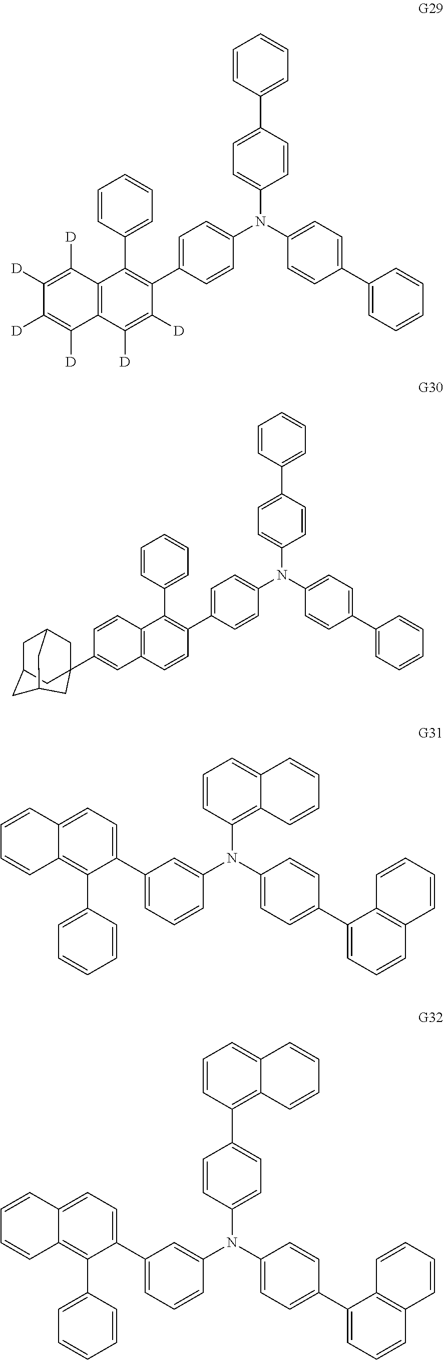 [SMILES: [2H]c1c([2H])c([2H])c2c(-c3ccccc3)c(-c3ccc(N(c4ccc(-c5ccccc5)cc4)c4ccc(-c5ccccc5)cc4)cc3)c([2H])c([2H])c2c1[2H] ; c1ccc(-c2ccc(N(c3ccc(-c4ccccc4)cc3)c3ccc(-c4ccc5cc(C67CC8CC(CC(C8)C6)C7)ccc5c4-c4ccccc4)cc3)cc2)cc1 ; c1ccc(-c2c(-c3cccc(N(c4ccc(-c5cccc6ccccc56)cc4)c4cccc5ccccc45)c3)ccc3ccccc23)cc1 ; c1ccc(-c2c(-c3cccc(N(c4ccc(-c5cccc6ccccc56)cc4)c4ccc(-c5cccc6ccccc56)cc4)c3)ccc3ccccc23)cc1]

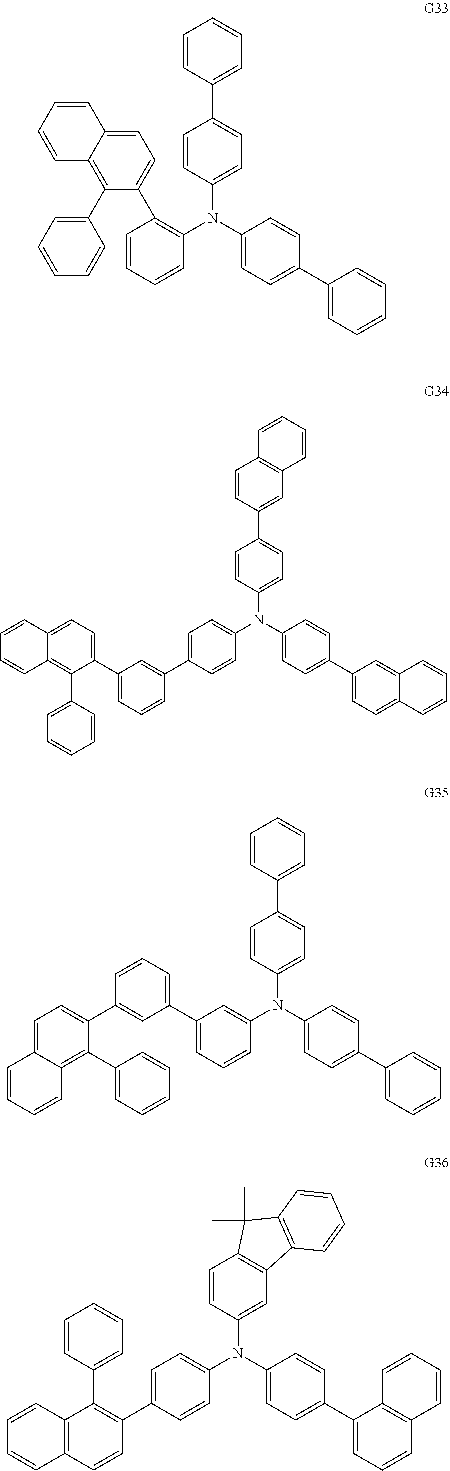 [SMILES: c1ccc(-c2ccc(N(c3ccc(-c4ccccc4)cc3)c3ccccc3-c3ccc4ccccc4c3-c3ccccc3)cc2)cc1 ; c1ccc(-c2c(-c3cccc(-c4ccc(N(c5ccc(-c6ccc7ccccc7c6)cc5)c5ccc(-c6ccc7ccccc7c6)cc5)cc4)c3)ccc3ccccc23)cc1 ; c1ccc(-c2ccc(N(c3ccc(-c4ccccc4)cc3)c3cccc(-c4cccc(-c5ccc6ccccc6c5-c5ccccc5)c4)c3)cc2)cc1 ; CC1(C)c2ccccc2-c2cc(N(c3ccc(-c4ccc5ccccc5c4-c4ccccc4)cc3)c3ccc(-c4cccc5ccccc45)cc3)ccc21]

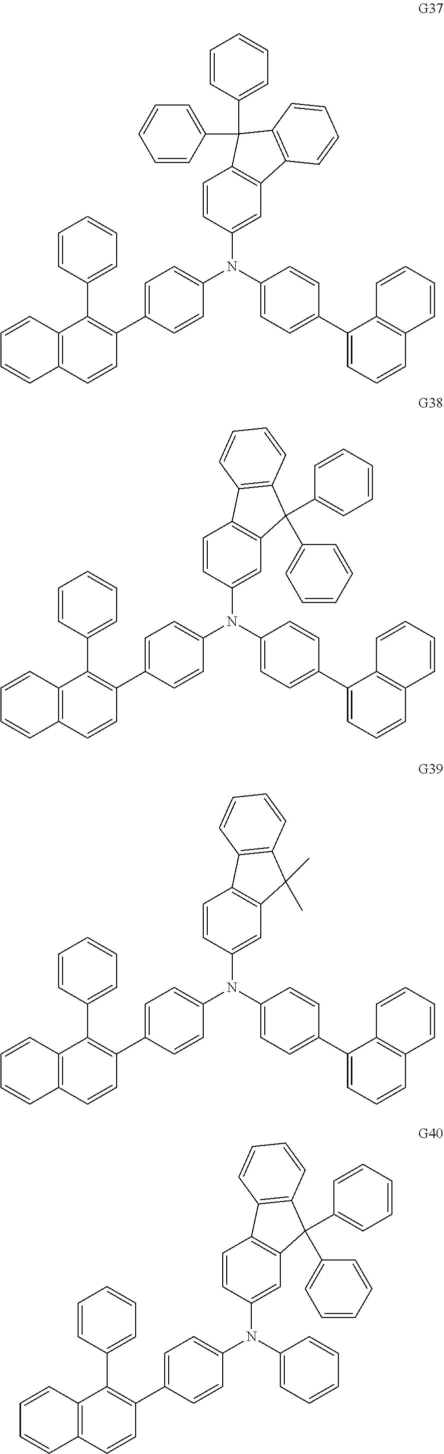 [SMILES: c1ccc(-c2c(-c3ccc(N(c4ccc(-c5cccc6ccccc56)cc4)c4ccc5c(c4)-c4ccccc4C5(c4ccccc4)c4ccccc4)cc3)ccc3ccccc23)cc1 ; c1ccc(-c2c(-c3ccc(N(c4ccc(-c5cccc6ccccc56)cc4)c4ccc5c(c4)C(c4ccccc4)(c4ccccc4)c4ccccc4-5)cc3)ccc3ccccc23)cc1 ; CC1(C)c2ccccc2-c2ccc(N(c3ccc(-c4ccc5ccccc5c4-c4ccccc4)cc3)c3ccc(-c4cccc5ccccc45)cc3)cc21 ; c1ccc(-c2c(-c3ccc(N(c4ccccc4)c4ccc5c(c4)C(c4ccccc4)(c4ccccc4)c4ccccc4-5)cc3)ccc3ccccc23)cc1]

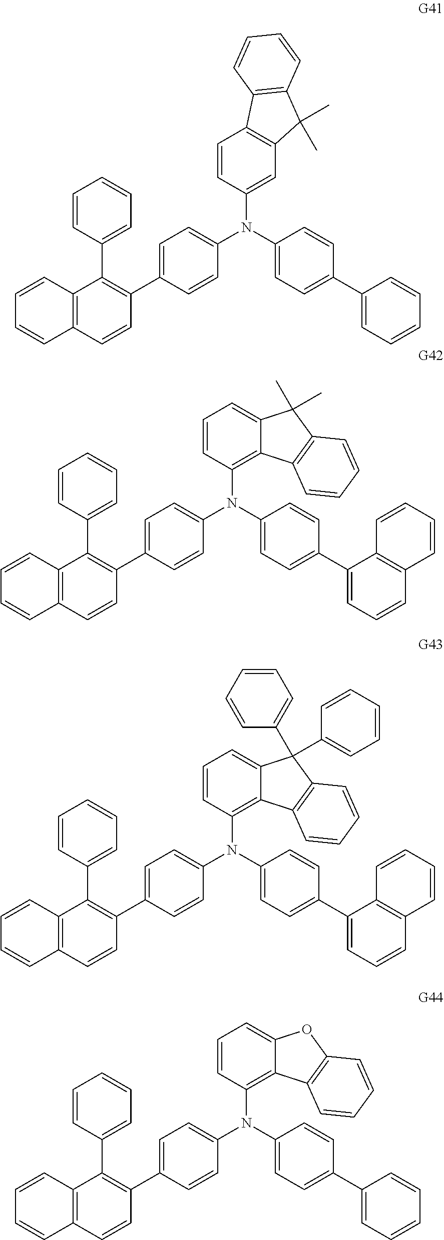 [SMILES: CC1(C)c2ccccc2-c2ccc(N(c3ccc(-c4ccccc4)cc3)c3ccc(-c4ccc5ccccc5c4-c4ccccc4)cc3)cc21 ; CC1(C)c2ccccc2-c2c(N(c3ccc(-c4ccc5ccccc5c4-c4ccccc4)cc3)c3ccc(-c4cccc5ccccc45)cc3)cccc21 ; c1ccc(-c2c(-c3ccc(N(c4ccc(-c5cccc6ccccc56)cc4)c4cccc5c4-c4ccccc4C5(c4ccccc4)c4ccccc4)cc3)ccc3ccccc23)cc1 ; c1ccc(-c2ccc(N(c3ccc(-c4ccc5ccccc5c4-c4ccccc4)cc3)c3cccc4oc5ccccc5c34)cc2)cc1]

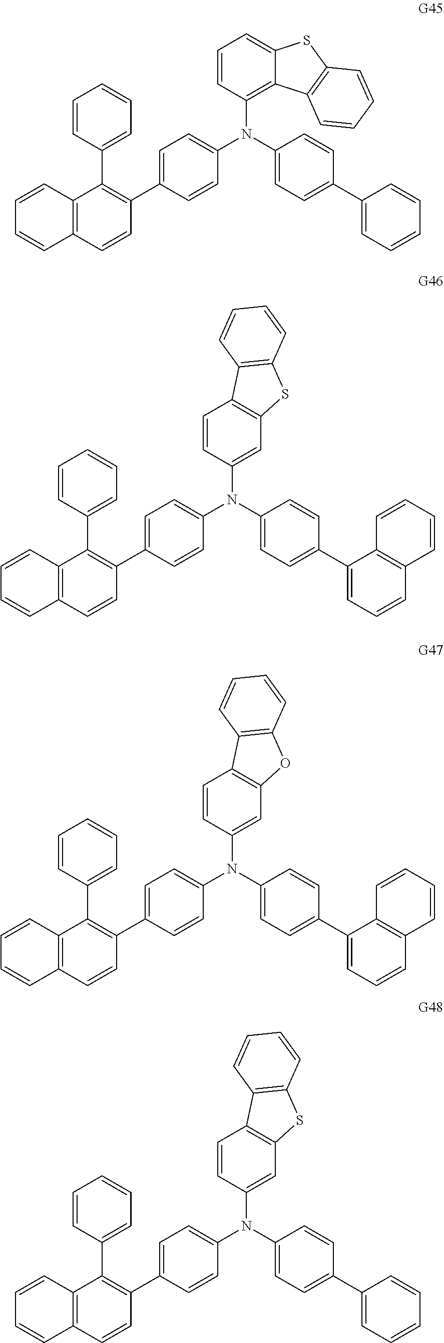 [SMILES: c1ccc(-c2ccc(N(c3ccc(-c4ccc5ccccc5c4-c4ccccc4)cc3)c3cccc4sc5ccccc5c34)cc2)cc1 ; c1ccc(-c2c(-c3ccc(N(c4ccc(-c5cccc6ccccc56)cc4)c4ccc5c(c4)sc4ccccc45)cc3)ccc3ccccc23)cc1 ; c1ccc(-c2c(-c3ccc(N(c4ccc(-c5cccc6ccccc56)cc4)c4ccc5c(c4)oc4ccccc45)cc3)ccc3ccccc23)cc1 ; c1ccc(-c2ccc(N(c3ccc(-c4ccc5ccccc5c4-c4ccccc4)cc3)c3ccc4c(c3)sc3ccccc34)cc2)cc1]

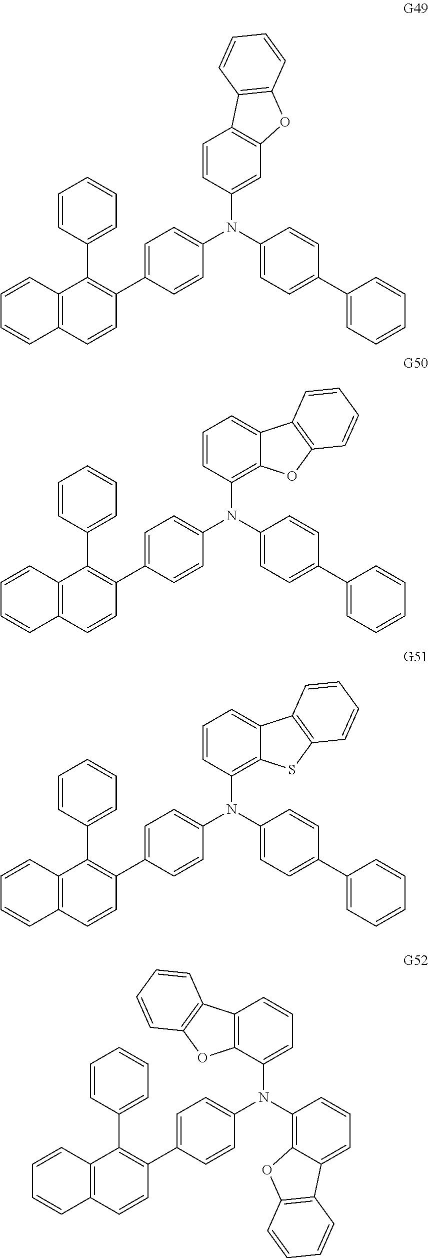 [SMILES: c1ccc(-c2ccc(N(c3ccc(-c4ccc5ccccc5c4-c4ccccc4)cc3)c3ccc4c(c3)oc3ccccc34)cc2)cc1 ; c1ccc(-c2ccc(N(c3ccc(-c4ccc5ccccc5c4-c4ccccc4)cc3)c3cccc4c3oc3ccccc34)cc2)cc1 ; c1ccc(-c2ccc(N(c3ccc(-c4ccc5ccccc5c4-c4ccccc4)cc3)c3cccc4c3sc3ccccc34)cc2)cc1 ; c1ccc(-c2c(-c3ccc(N(c4cccc5c4oc4ccccc45)c4cccc5c4oc4ccccc45)cc3)ccc3ccccc23)cc1]

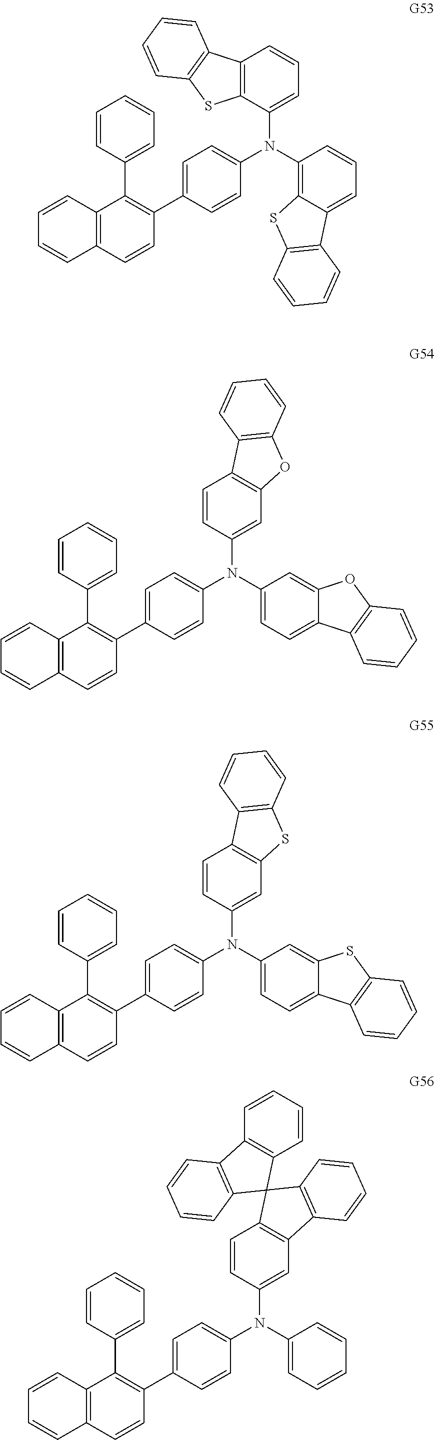 [SMILES: c1ccc(-c2c(-c3ccc(N(c4cccc5c4sc4ccccc45)c4cccc5c4sc4ccccc45)cc3)ccc3ccccc23)cc1 ; c1ccc(-c2c(-c3ccc(N(c4ccc5c(c4)oc4ccccc45)c4ccc5c(c4)oc4ccccc45)cc3)ccc3ccccc23)cc1 ; c1ccc(-c2c(-c3ccc(N(c4ccc5c(c4)sc4ccccc45)c4ccc5c(c4)sc4ccccc45)cc3)ccc3ccccc23)cc1 ; c1ccc(-c2c(-c3ccc(N(c4ccccc4)c4ccc5c(c4)-c4ccccc4C54c5ccccc5-c5ccccc54)cc3)ccc3ccccc23)cc1]

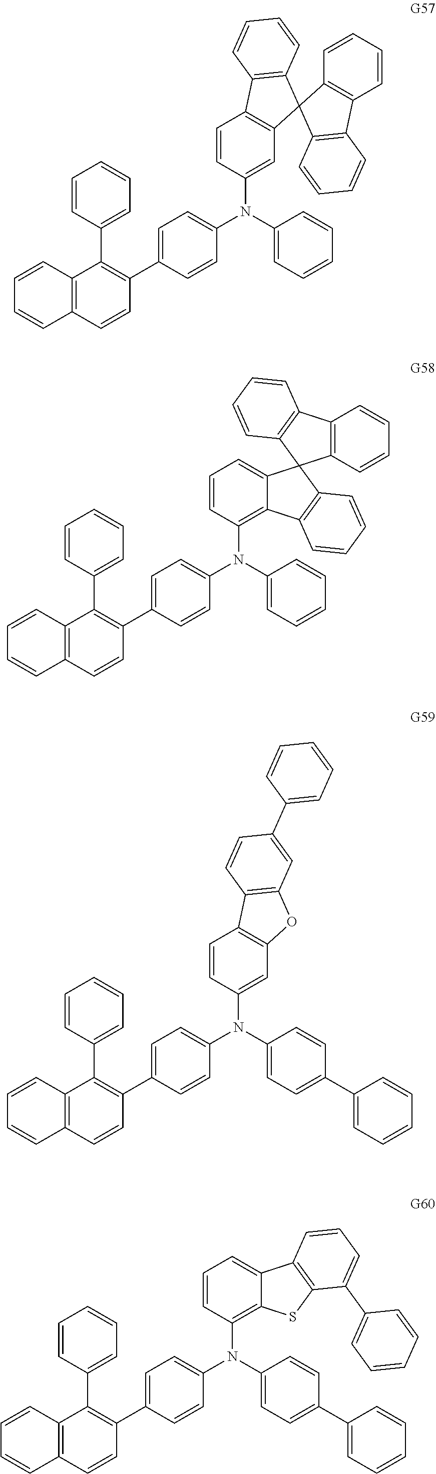 [SMILES: c1ccc(-c2c(-c3ccc(N(c4ccccc4)c4ccc5c(c4)C4(c6ccccc6-c6ccccc64)c4ccccc4-5)cc3)ccc3ccccc23)cc1 ; c1ccc(-c2c(-c3ccc(N(c4ccccc4)c4cccc5c4-c4ccccc4C54c5ccccc5-c5ccccc54)cc3)ccc3ccccc23)cc1 ; c1ccc(-c2ccc(N(c3ccc(-c4ccc5ccccc5c4-c4ccccc4)cc3)c3ccc4c(c3)oc3cc(-c5ccccc5)ccc34)cc2)cc1 ; c1ccc(-c2ccc(N(c3ccc(-c4ccc5ccccc5c4-c4ccccc4)cc3)c3cccc4c3sc3c(-c5ccccc5)cccc34)cc2)cc1]

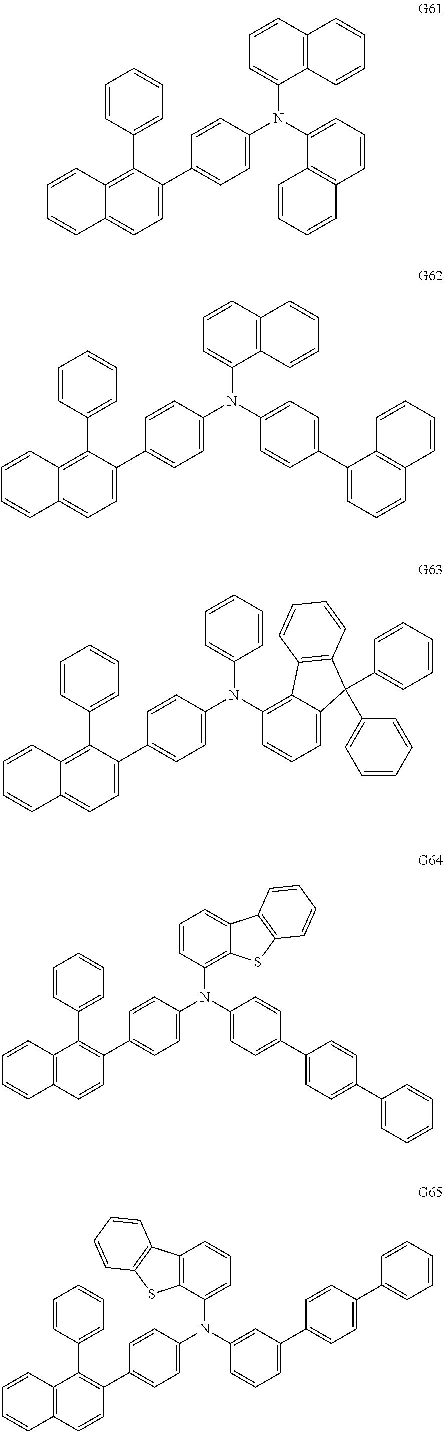 [SMILES: c1ccc(-c2c(-c3ccc(N(c4cccc5ccccc45)c4cccc5ccccc45)cc3)ccc3ccccc23)cc1 ; c1ccc(-c2c(-c3ccc(N(c4ccc(-c5cccc6ccccc56)cc4)c4cccc5ccccc45)cc3)ccc3ccccc23)cc1 ; c1ccc(-c2c(-c3ccc(N(c4ccccc4)c4cccc5c4-c4ccccc4C5(c4ccccc4)c4ccccc4)cc3)ccc3ccccc23)cc1 ; c1ccc(-c2ccc(-c3ccc(N(c4ccc(-c5ccc6ccccc6c5-c5ccccc5)cc4)c4cccc5c4sc4ccccc45)cc3)cc2)cc1 ; c1ccc(-c2ccc(-c3cccc(N(c4ccc(-c5ccc6ccccc6c5-c5ccccc5)cc4)c4cccc5c4sc4ccccc45)c3)cc2)cc1]

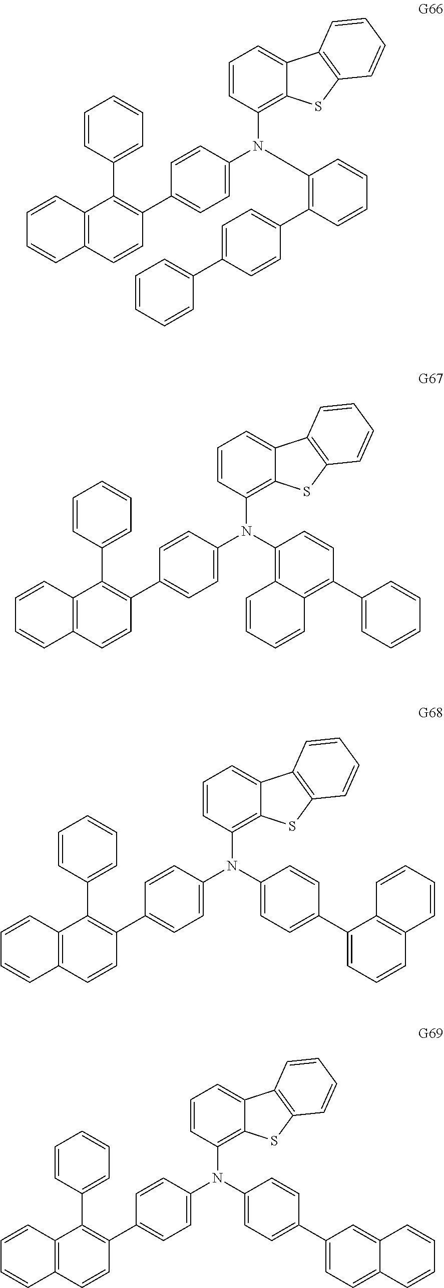 [SMILES: c1ccc(-c2ccc(-c3ccccc3N(c3ccc(-c4ccc5ccccc5c4-c4ccccc4)cc3)c3cccc4c3sc3ccccc34)cc2)cc1 ; c1ccc(-c2c(-c3ccc(N(c4ccc(-c5ccccc5)c5ccccc45)c4cccc5c4sc4ccccc45)cc3)ccc3ccccc23)cc1 ; c1ccc(-c2c(-c3ccc(N(c4ccc(-c5cccc6ccccc56)cc4)c4cccc5c4sc4ccccc45)cc3)ccc3ccccc23)cc1 ; c1ccc(-c2c(-c3ccc(N(c4ccc(-c5ccc6ccccc6c5)cc4)c4cccc5c4sc4ccccc45)cc3)ccc3ccccc23)cc1]

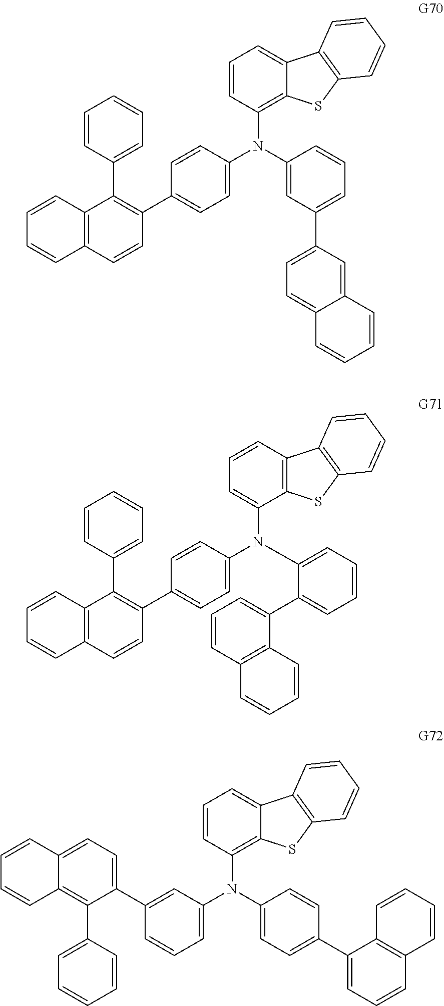 [SMILES: c1ccc(-c2c(-c3ccc(N(c4cccc(-c5ccc6ccccc6c5)c4)c4cccc5c4sc4ccccc45)cc3)ccc3ccccc23)cc1 ; c1ccc(-c2c(-c3ccc(N(c4ccccc4-c4cccc5ccccc45)c4cccc5c4sc4ccccc45)cc3)ccc3ccccc23)cc1 ; c1ccc(-c2c(-c3cccc(N(c4ccc(-c5cccc6ccccc56)cc4)c4cccc5c4sc4ccccc45)c3)ccc3ccccc23)cc1]

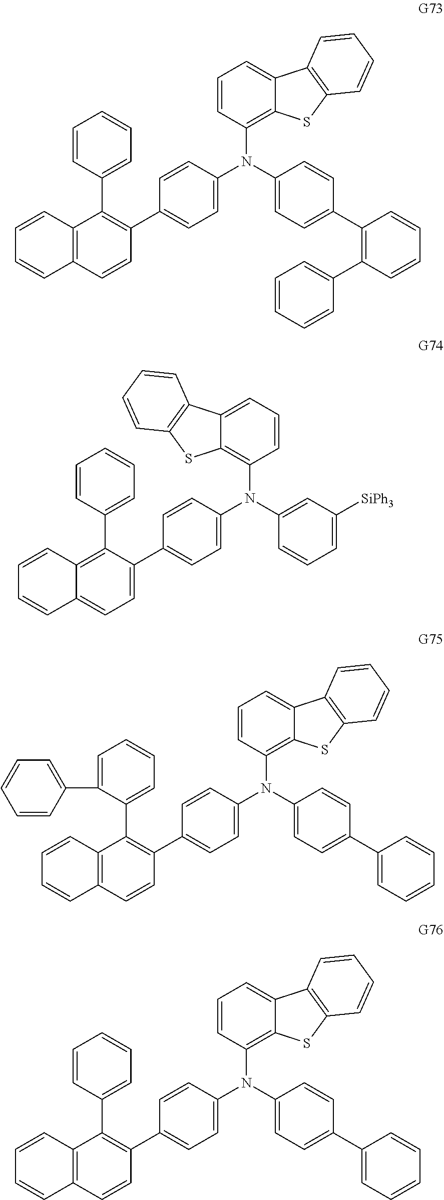

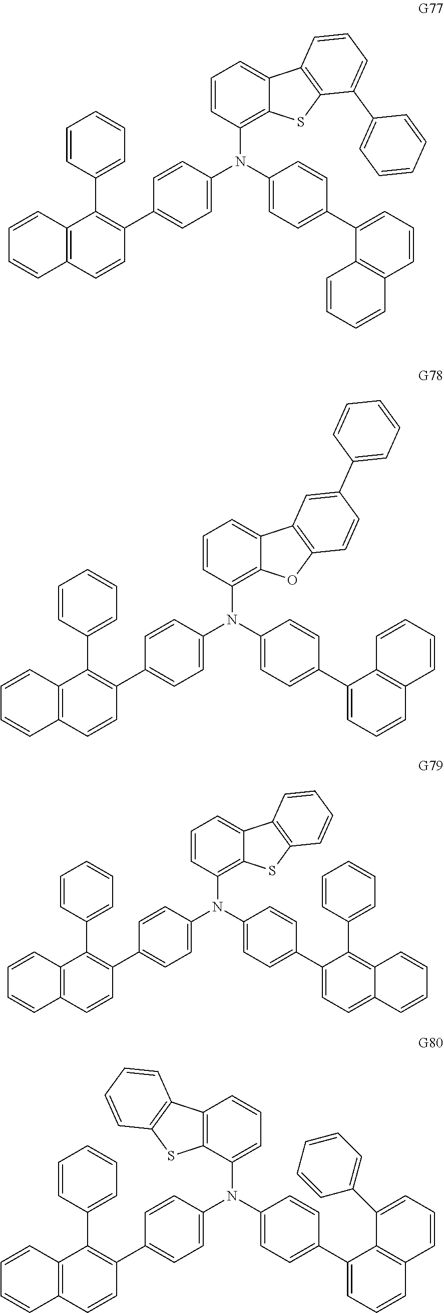 [SMILES: c1ccc(-c2c(-c3ccc(N(c4ccc(-c5cccc6ccccc56)cc4)c4cccc5c4sc4c(-c6ccccc6)cccc45)cc3)ccc3ccccc23)cc1 ; c1ccc(-c2ccc3oc4c(N(c5ccc(-c6ccc7ccccc7c6-c6ccccc6)cc5)c5ccc(-c6cccc7ccccc67)cc5)cccc4c3c2)cc1 ; c1ccc(-c2c(-c3ccc(N(c4ccc(-c5ccc6ccccc6c5-c5ccccc5)cc4)c4cccc5c4sc4ccccc45)cc3)ccc3ccccc23)cc1 ; c1ccc(-c2c(-c3ccc(N(c4ccc(-c5cccc6cccc(-c7ccccc7)c56)cc4)c4cccc5c4sc4ccccc45)cc3)ccc3ccccc23)cc1]

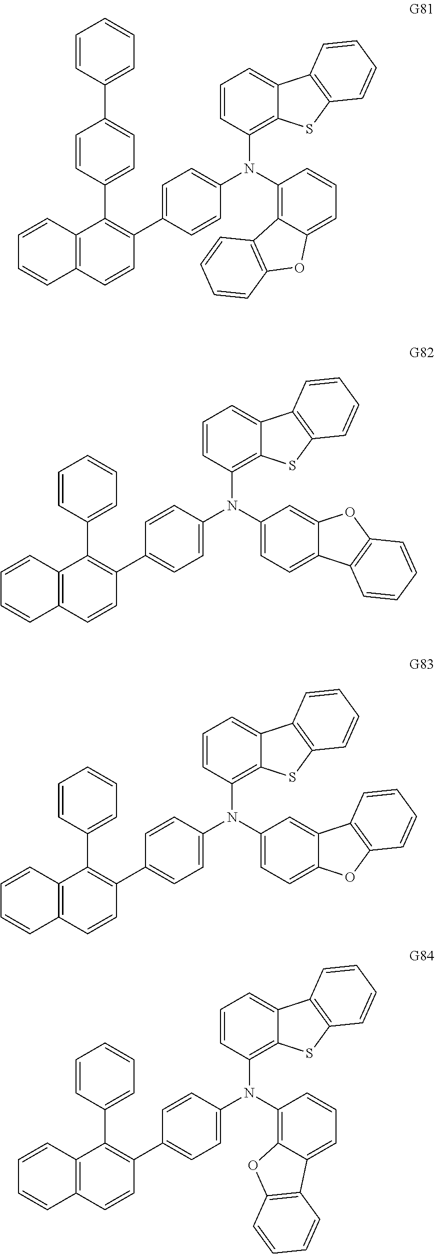 [SMILES: c1ccc(-c2ccc(-c3c(-c4ccc(N(c5cccc6c5sc5ccccc56)c5cccc6oc7ccccc7c56)cc4)ccc4ccccc34)cc2)cc1 ; c1ccc(-c2c(-c3ccc(N(c4ccc5c(c4)oc4ccccc45)c4cccc5c4sc4ccccc45)cc3)ccc3ccccc23)cc1 ; c1ccc(-c2c(-c3ccc(N(c4ccc5oc6ccccc6c5c4)c4cccc5c4sc4ccccc45)cc3)ccc3ccccc23)cc1 ; c1ccc(-c2c(-c3ccc(N(c4cccc5c4oc4ccccc45)c4cccc5c4sc4ccccc45)cc3)ccc3ccccc23)cc1]

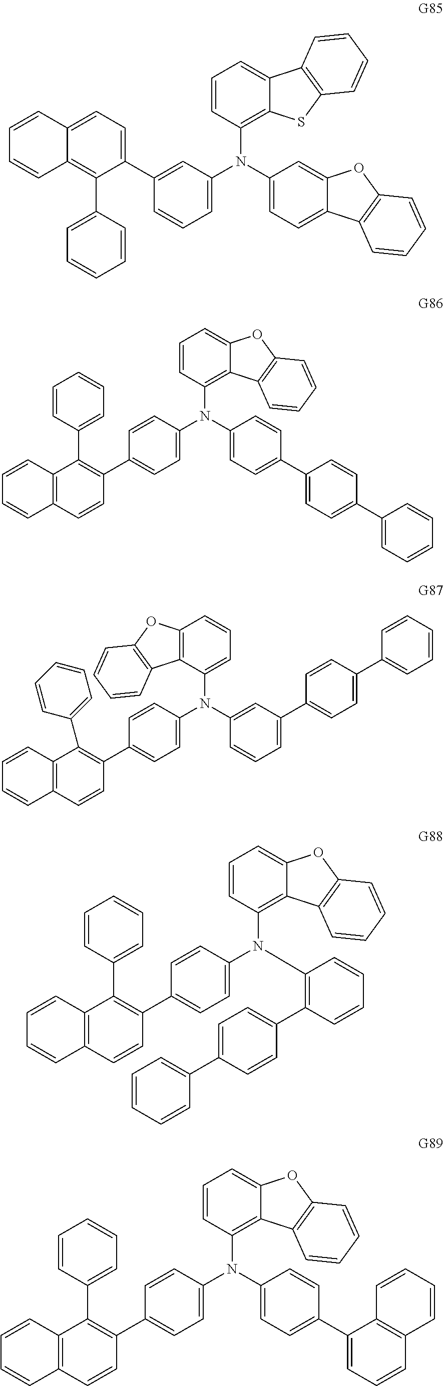 [SMILES: c1ccc(-c2c(-c3cccc(N(c4ccc5c(c4)oc4ccccc45)c4cccc5c4sc4ccccc45)c3)ccc3ccccc23)cc1 ; c1ccc(-c2ccc(-c3ccc(N(c4ccc(-c5ccc6ccccc6c5-c5ccccc5)cc4)c4cccc5oc6ccccc6c45)cc3)cc2)cc1 ; c1ccc(-c2ccc(-c3cccc(N(c4ccc(-c5ccc6ccccc6c5-c5ccccc5)cc4)c4cccc5oc6ccccc6c45)c3)cc2)cc1 ; c1ccc(-c2ccc(-c3ccccc3N(c3ccc(-c4ccc5ccccc5c4-c4ccccc4)cc3)c3cccc4oc5ccccc5c34)cc2)cc1 ; c1ccc(-c2c(-c3ccc(N(c4ccc(-c5cccc6ccccc56)cc4)c4cccc5oc6ccccc6c45)cc3)ccc3ccccc23)cc1]

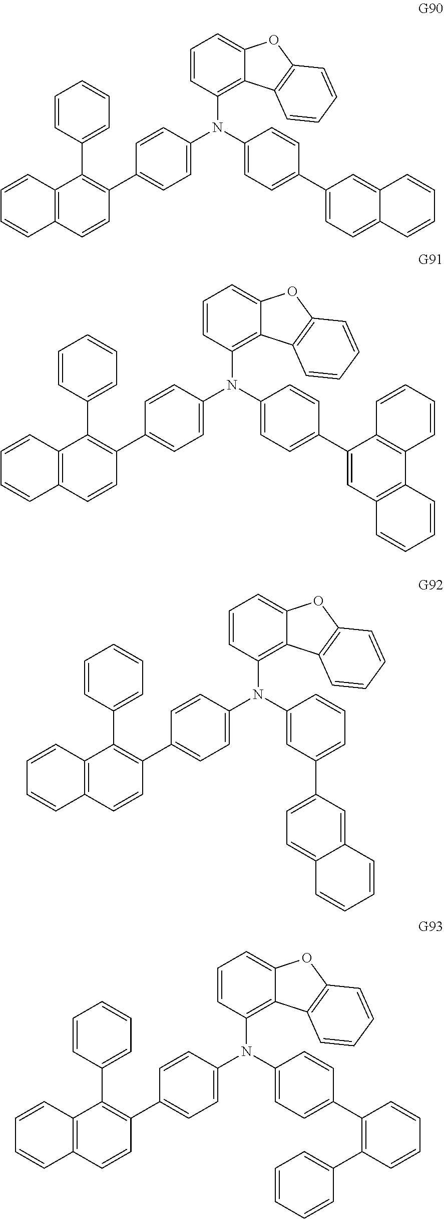 [SMILES: c1ccc(-c2c(-c3ccc(N(c4ccc(-c5ccc6ccccc6c5)cc4)c4cccc5oc6ccccc6c45)cc3)ccc3ccccc23)cc1 ; c1ccc(-c2c(-c3ccc(N(c4ccc(-c5cc6ccccc6c6ccccc56)cc4)c4cccc5oc6ccccc6c45)cc3)ccc3ccccc23)cc1 ; c1ccc(-c2c(-c3ccc(N(c4cccc(-c5ccc6ccccc6c5)c4)c4cccc5oc6ccccc6c45)cc3)ccc3ccccc23)cc1 ; c1ccc(-c2ccccc2-c2ccc(N(c3ccc(-c4ccc5ccccc5c4-c4ccccc4)cc3)c3cccc4oc5ccccc5c34)cc2)cc1]

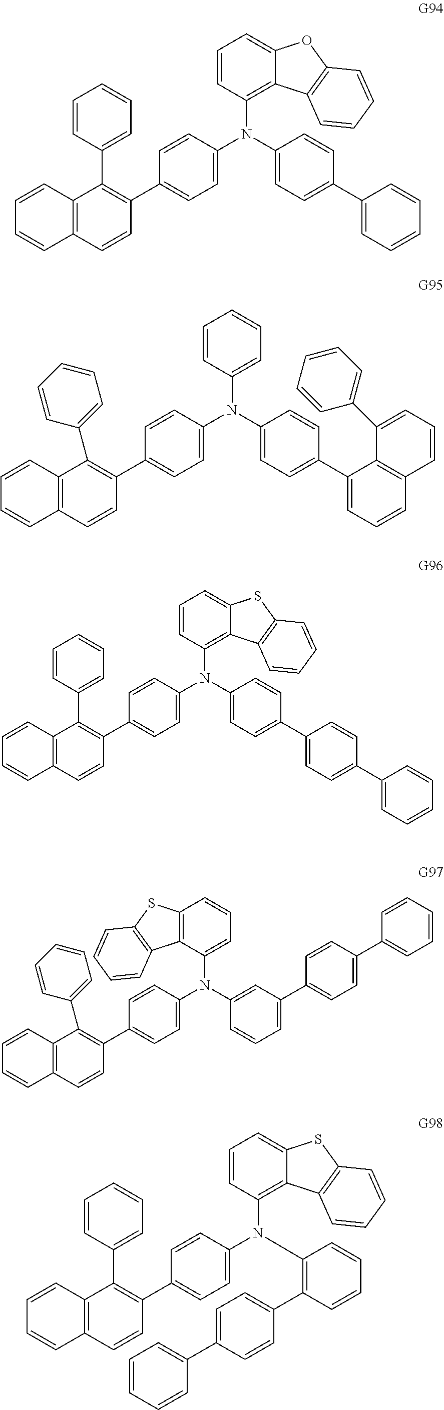 [SMILES: c1ccc(-c2ccc(N(c3ccc(-c4ccc5ccccc5c4-c4ccccc4)cc3)c3cccc4oc5ccccc5c34)cc2)cc1 ; c1ccc(-c2c(-c3ccc(N(c4ccccc4)c4ccc(-c5cccc6cccc(-c7ccccc7)c56)cc4)cc3)ccc3ccccc23)cc1 ; c1ccc(-c2ccc(-c3ccc(N(c4ccc(-c5ccc6ccccc6c5-c5ccccc5)cc4)c4cccc5sc6ccccc6c45)cc3)cc2)cc1 ; c1ccc(-c2ccc(-c3cccc(N(c4ccc(-c5ccc6ccccc6c5-c5ccccc5)cc4)c4cccc5sc6ccccc6c45)c3)cc2)cc1 ; c1ccc(-c2ccc(-c3ccccc3N(c3ccc(-c4ccc5ccccc5c4-c4ccccc4)cc3)c3cccc4sc5ccccc5c34)cc2)cc1]

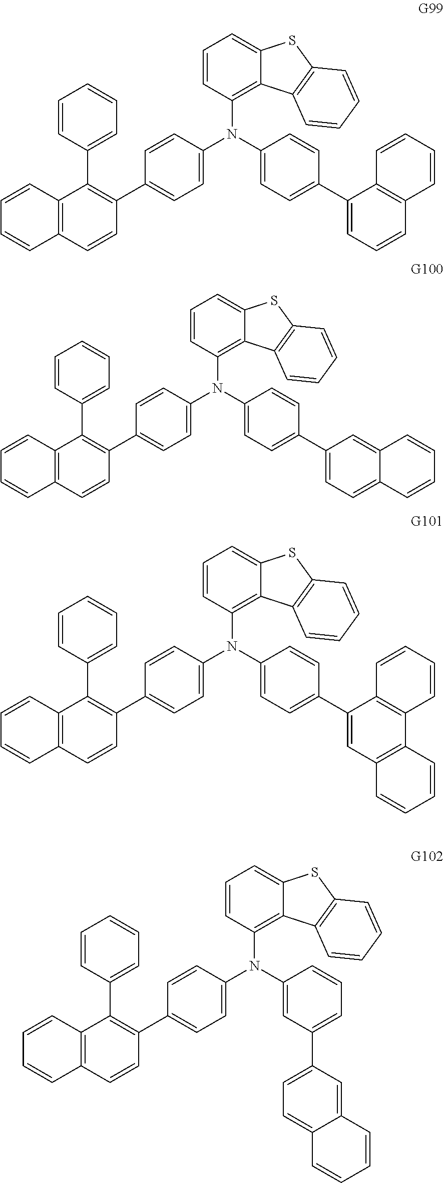 [SMILES: c1ccc(-c2c(-c3ccc(N(c4ccc(-c5cccc6ccccc56)cc4)c4cccc5sc6ccccc6c45)cc3)ccc3ccccc23)cc1 ; c1ccc(-c2c(-c3ccc(N(c4ccc(-c5ccc6ccccc6c5)cc4)c4cccc5sc6ccccc6c45)cc3)ccc3ccccc23)cc1 ; c1ccc(-c2c(-c3ccc(N(c4ccc(-c5cc6ccccc6c6ccccc56)cc4)c4cccc5sc6ccccc6c45)cc3)ccc3ccccc23)cc1 ; c1ccc(-c2c(-c3ccc(N(c4cccc(-c5ccc6ccccc6c5)c4)c4cccc5sc6ccccc6c45)cc3)ccc3ccccc23)cc1]

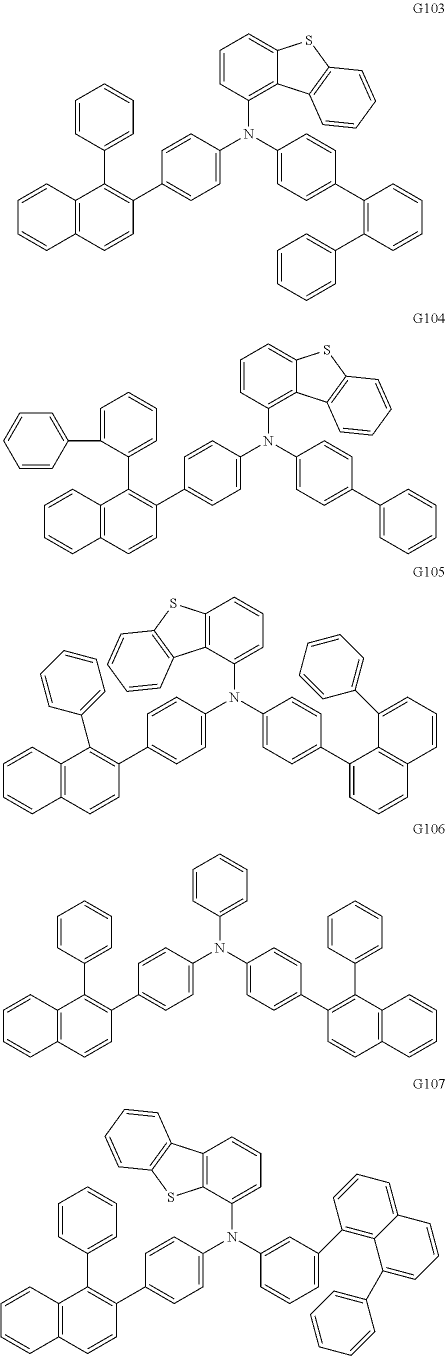 [SMILES: c1ccc(-c2ccccc2-c2ccc(N(c3ccc(-c4ccc5ccccc5c4-c4ccccc4)cc3)c3cccc4sc5ccccc5c34)cc2)cc1 ; c1ccc(-c2ccc(N(c3ccc(-c4ccc5ccccc5c4-c4ccccc4-c4ccccc4)cc3)c3cccc4sc5ccccc5c34)cc2)cc1 ; c1ccc(-c2c(-c3ccc(N(c4ccc(-c5cccc6cccc(-c7ccccc7)c56)cc4)c4cccc5sc6ccccc6c45)cc3)ccc3ccccc23)cc1 ; c1ccc(-c2c(-c3ccc(N(c4ccccc4)c4ccc(-c5ccc6ccccc6c5-c5ccccc5)cc4)cc3)ccc3ccccc23)cc1 ; c1ccc(-c2c(-c3ccc(N(c4cccc(-c5cccc6cccc(-c7ccccc7)c56)c4)c4cccc5c4sc4ccccc45)cc3)ccc3ccccc23)cc1]

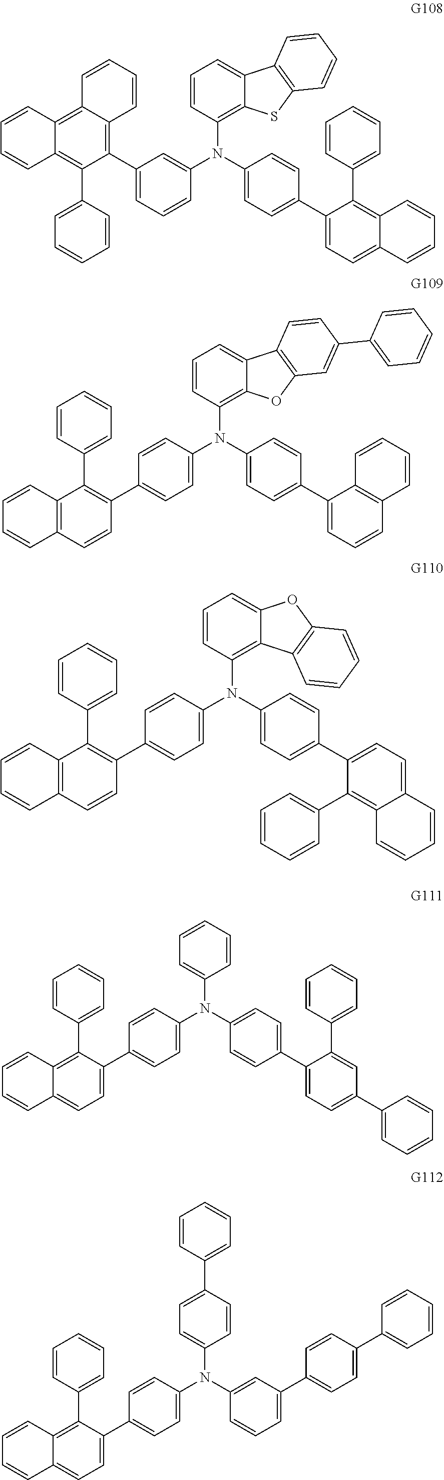 [SMILES: c1ccc(-c2c(-c3ccc(N(c4cccc(-c5c(-c6ccccc6)c6ccccc6c6ccccc56)c4)c4cccc5c4sc4ccccc45)cc3)ccc3ccccc23)cc1 ; c1ccc(-c2ccc3c(c2)oc2c(N(c4ccc(-c5ccc6ccccc6c5-c5ccccc5)cc4)c4ccc(-c5cccc6ccccc56)cc4)cccc23)cc1 ; c1ccc(-c2c(-c3ccc(N(c4ccc(-c5ccc6ccccc6c5-c5ccccc5)cc4)c4cccc5oc6ccccc6c45)cc3)ccc3ccccc23)cc1 ; c1ccc(-c2ccc(-c3ccc(N(c4ccccc4)c4ccc(-c5ccc6ccccc6c5-c5ccccc5)cc4)cc3)c(-c3ccccc3)c2)cc1 ; c1ccc(-c2ccc(-c3cccc(N(c4ccc(-c5ccccc5)cc4)c4ccc(-c5ccc6ccccc6c5-c5ccccc5)cc4)c3)cc2)cc1]

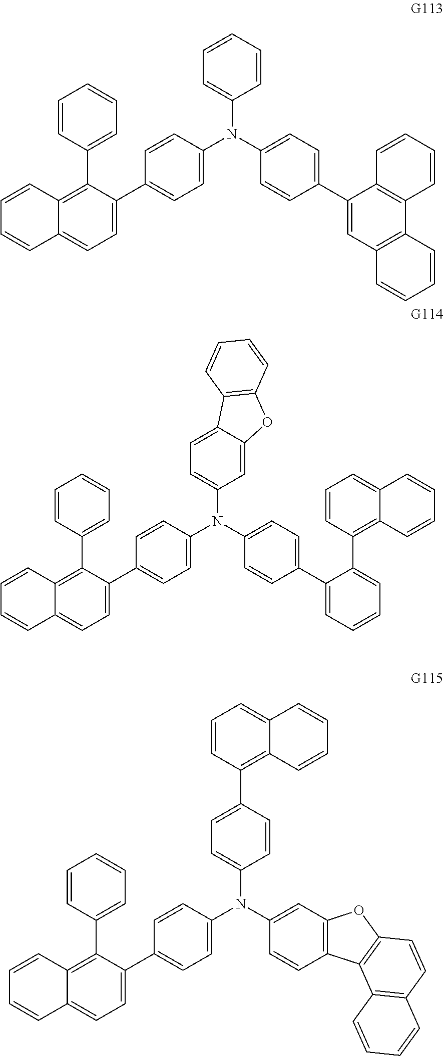 [SMILES: c1ccc(-c2c(-c3ccc(N(c4ccccc4)c4ccc(-c5cc6ccccc6c6ccccc56)cc4)cc3)ccc3ccccc23)cc1 ; c1ccc(-c2c(-c3ccc(N(c4ccc(-c5ccccc5-c5cccc6ccccc56)cc4)c4ccc5c(c4)oc4ccccc45)cc3)ccc3ccccc23)cc1 ; c1ccc(-c2c(-c3ccc(N(c4ccc(-c5cccc6ccccc56)cc4)c4ccc5c(c4)oc4ccc6ccccc6c45)cc3)ccc3ccccc23)cc1]

The monoamine compound according to an example embodiment includes may include a fused ring and a phenylnaphthyl group with a high thermal resistance and electric charge resistance, and may help to extend a device life when used as a material for an organic electroluminescence device. When used as a material for an organic electroluminescence device, the monoamine compound may enhance the quality of layers due to the bulky phenylnaphthyl group which decreases symmetry of molecule and inhibits crystallization, thereby contributing to securing high efficiency.

Hereinafter, an organic electroluminescence device according to an example embodiment will be explained, referring to FIGS. 1 to 3. The organic electroluminescence device according to an example embodiment includes the monoamine compound according to an example embodiment. For example, a hole transport region HTR includes the monoamine compound represented by Formula 1.

The following explanation will be mainly given with features different from the monoamine compound according to an example embodiment, and unexplained parts will follow the above description on the monoamine compound according to an example embodiment.

The first electrode EL1 has conductivity. The first electrode EL1 may be a pixel electrode or an anode. The first electrode EL1 may be a transmissive electrode, a transflective electrode, or a reflective electrode. In case the first electrode EL1 is the transmissive electrode, the first electrode EL1 may include a transparent metal oxide such as indium tin oxide (ITO), indium zinc oxide (IZO), zinc oxide (ZnO), or indium tin zinc oxide (ITZO). In case the first electrode EL1 is the transflective electrode or reflective electrode, the first electrode EL1 may include Ag, Mg, Cu, Al, Pt, Pd, Au, Ni, Nd, Ir, Cr, Li, Ca, LiF/Ca, LiF/Al, Mo, Ti, a compound thereof, or a mixture thereof (for example, a mixture of Ag and Mg). Also, the first electrode EL1 may have a structure including a plurality of layers including a reflective layer or transflective layer formed using the above materials, and a transparent conductive layer formed using ITO, IZO, ZnO, or ITZO. For example, the first electrode EL1 may have a triple-layer structure of ITO/Ag/ITO.

The thickness of the first electrode EL1 may be from about 1,000 Å to about 10,000 Å, for example, from about 1,000 Å to about 3,000 Å.

The hole transport region HTR is on the first electrode EL1. The hole transport region HTR may include at least one of a hole injection layer HIL, a hole transport layer HTL, a hole buffer layer, or an electron blocking layer EBL.

The hole transport region HTR includes the monoamine compound according to an example embodiment, as described above.

The hole transport region HTR may have a single layer formed using a single material, a single layer formed using a plurality of different materials, or a multilayer structure including a plurality of layers formed using a plurality of different materials.

For example, the hole transport region HTR may have a single layer structure of a hole injection layer HIL or a hole transport layer HTL, or may have a single layer structure formed using a hole injection material and a hole transport material. In addition, the hole transport region HTR may have a single layer structure formed using a plurality of different materials, or a laminated structure of hole injection layer HIL/hole transport layer HTL, hole injection layer HIL/hole transport layer HTL/hole buffer layer, hole injection layer HIL/hole buffer layer, hole transport layer HTL/hole buffer layer, or hole injection layer HIL/hole transport layer HTL/electron blocking layer EBL, laminated in order from the first electrode EL1, without limitation.

As described above, the hole transport region HTR may have a multilayer structure having a plurality of layers, and a layer of the plurality of layers contacting the emission layer EML may include the monoamine compound represented by Formula 1. For example, the hole transport region HTR may include a hole injection layer HIL on the first electrode EL1, a hole transport layer HTL on the hole injection layer HIL, and an electron blocking layer EBL on the hole transport layer HTL, and the electron blocking layer EBL may include the monoamine compound represented by Formula 1. In another example, the hole transport region HTR may include a hole injection layer HIL and a hole transport layer HTL, and the hole transport layer HTL may include the monoamine compound represented by Formula 1.

The hole transport region HTR may include one or more of the monoamine compound represented by Formula 1. For example, the hole transport region HTR may include at least one selected from the group of compounds represented in the above-described Compound Groups 1 to 7.

The hole transport region HTR may be formed using various methods such as a vacuum deposition method, a spin coating method, a cast method, a Langmuir-Blodgett (LB) method, an inkjet printing method, a laser printing method, and a laser induced thermal imaging (LITI) method.

Furthermore, the hole transport region HTR may include the following materials in each layer.

The hole injection layer HIL may include, for example, a phthalocyanine compound such as copper phthalocyanine; N,N′-diphenyl-N,N′-bis-[4-(phenyl-m-tolyl-amino)-phenyl]-biphenyl-4,4′-diamine (DNTPD), 4,4′,4″-tris(3-methylphenylphenylamino)triphenylamine (m-MTDATA), 4,4′,4″-tris(N,N-diphenylamino)triphenylamine (TDATA), 4,4′,4″-tris{N-(2-naphthyl)-N-phenylamino}-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalen-1-yl)-N,N′-diphenyl-benzidine (NPD), triphenylamine-containing polyether ketone (TPAPEK), 4-isopropyl-4′-methyldiphenyliodonium tetrakis(pentafluorophenyl)borate, dipyrazino[2,3-f: 2′,3′-h] quinoxaline-2,3,6,7,10,11-hexacarbonitrile (HAT-CN), etc.

The hole transport layer HTL may include, for example, carbazole derivatives such as N-phenyl carbazole, polyvinyl carbazole, fluorine-based derivatives, N,N′-bis(3-methylphenyl)-N,N′-diphenyl-[1,1-biphenyl]-4,4′-diamine (TPD), triphenylamine-based derivatives such as 4,4′,4″-tris(N-carbazolyl)triphenylamine (TCTA), N,N′-di(naphthalen-1-yl)-N,N′-diphenyl-benzidine (NPD), 4,4′-cyclohexylidene bis[N,N-bis(4-methylphenyl)benzenamine] (TAPC), 4,4′-bis[N,N′-(3-tolyl)amino]-3,3′-dimethylbiphenyl (HMTPD), etc.

The electron blocking layer EBL may include the monoamine compound represented by Formula 1, as described above. The electron blocking layer EBL may include a suitable material. The electron blocking layer EBL may include, for example, carbazole derivatives such as N-phenyl carbazole, polyvinyl carbazole, fluorine-based derivatives, N,N′-bis(3-methylphenyl)-N,N′-diphenyl-[1,1-biphenyl]-4,4′-diamine (TPD), triphenylamine-based derivatives such as 4,4′,4″-tris(N-carbazolyl)triphenylamine (TCTA), N,N′-di(naphthalen-1-yl)-N,N′-diphenyl-benzidine (NPD), 4,4′-cyclohexylidene bis[N,N-bis(4-methylphenyl)benzenamine] (TAPC), 4,4′-bis[N,N′-(3-tolyl)amino]-3,3′-dimethylbiphenyl (HMTPD) or mCP, etc.

The thickness of the hole transport region HTR may be from about 100 Å to about 10,000 Å, for example, from about 100 Å to about 5,000 Å. The thickness of the hole injection layer HIL may be, for example, from about 30 Å to about 1,000 Å, and the thickness of the hole transport layer HTL may be from about 30 Å to about 1,000 Å. For example, the thickness of the electron blocking layer EBL may be from about 10 Å to about 1,000 Å. In case the thicknesses of the hole transport region HTR, the hole injection layer HIL, the hole transport layer HTL and the electron blocking layer EBL satisfy the above-described ranges, satisfactory hole transport properties may be obtained without substantial increase of a driving voltage.

The hole transport region HTR may further include a charge generating material in addition to the above-described materials to improve conductivity. The charge generating material may be dispersed in the hole transport region HTR uniformly or non-uniformly. The charge generating material may be, for example, a p-dopant. The p-dopant may be one of quinone derivatives, metal oxides, or cyano group-containing compounds, without limitation. For example, non-limiting examples of the p-dopant may include quinone derivatives such as tetracyanoquinodimethane (TCNQ), and 2,3,5,6-tetrafluoro-tetracyanoquinodimethane (F4-TCNQ), metal oxides such as tungsten oxide and molybdenum oxide, without limitation.

As described above, the hole transport region HTR may further include at least one of a hole buffer layer or an electron blocking layer EBL. The hole buffer layer may compensate an optical resonance distance according to the wavelength of light emitted from the emission layer EML and increase light emission efficiency. Materials included in the hole transport region HTR may be used as materials included in the hole buffer layer. The electron blocking layer EBL is a layer preventing electron injection from the electron transport region ETR into the hole transport region HTR.

The emission layer EML is on the hole transport region HTR. The thickness of the emission layer EML may be, for example, from about 100 Å to about 1,000 Å, or from about 100 Å to about 600 Å. The emission layer EML may have a single layer formed using a single material, a single layer formed using a plurality of different materials, or a multilayer structure having a plurality of layers formed using a plurality of different materials.

A suitable emission material may be used as a material for the emission layer EML. The material for the emission layer EML may be selected from, for example, fluoranthene derivatives, pyrene derivatives, arylacetylene derivatives, anthracene derivatives, fluorene derivatives, perylene derivatives, chrysene derivatives, or the like, and preferably, from pyrene derivatives, perylene derivatives, or anthracene derivatives. For example, as the host material of the emission layer EML, anthracene derivatives represented by the following Formula 10 may be used.

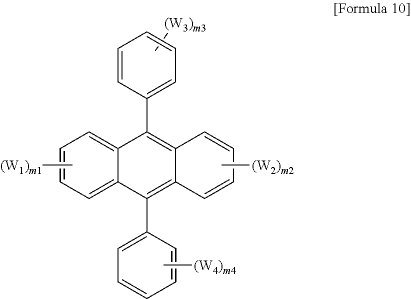

In Formula 10, W₁ to W₄ are each independently a hydrogen atom, a deuterium atom, a halogen atom, a substituted or unsubstituted silyl group, a substituted or unsubstituted alkyl group having 1 to 20 carbon atoms, a substituted or unsubstituted aryl group having 6 to 30 ring carbon atoms, or a substituted or unsubstituted heteroaryl group having 2 to 30 ring carbon atoms, or may form a ring by combining adjacent groups with each other, m₁ and m₂ are each independently an integer of 0 to 4, and m₁ and m₄ are each independently an integer of 0 to 5.

When m₁ is 1, W₁ may not be a hydrogen atom. When m₂ is 1, W₂ may not be a hydrogen atom. When m3 is 1, W₃ may not be a hydrogen atom. When m4 is 1, W₄ may not be a hydrogen atom.

When m1 is an integer of 2 or more, a plurality of W₁ may be the same or different from each other. When m2 is an integer of 2 or more, a plurality of W₂ may be the same or different from each other. When m3 is an integer of 2 or more, a plurality of W₃ may be the same or different from each other. When m4 is an integer of 2 or more, a plurality of W₄ may be the same or different from each other.

The compound represented by Formula 10 may include the compounds represented by the following structures, for example.

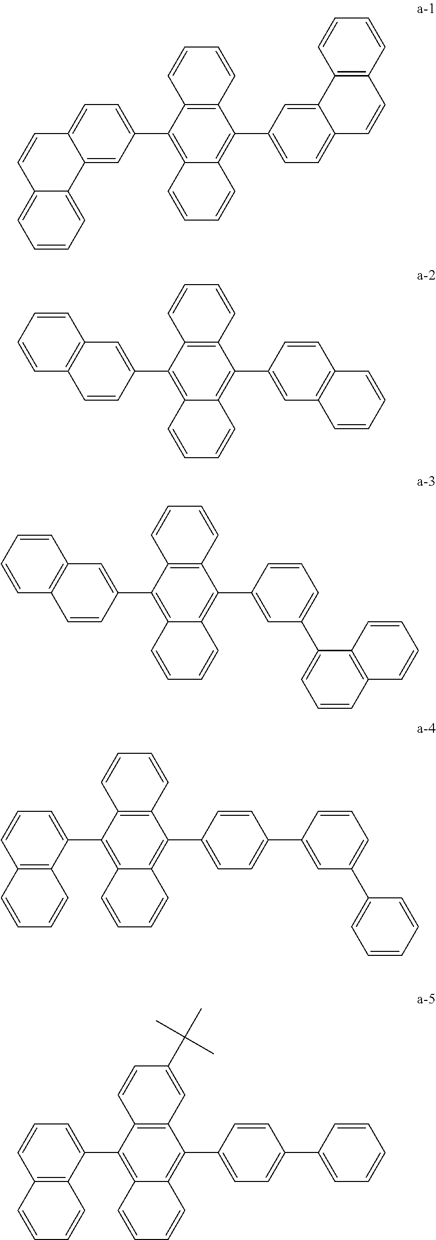

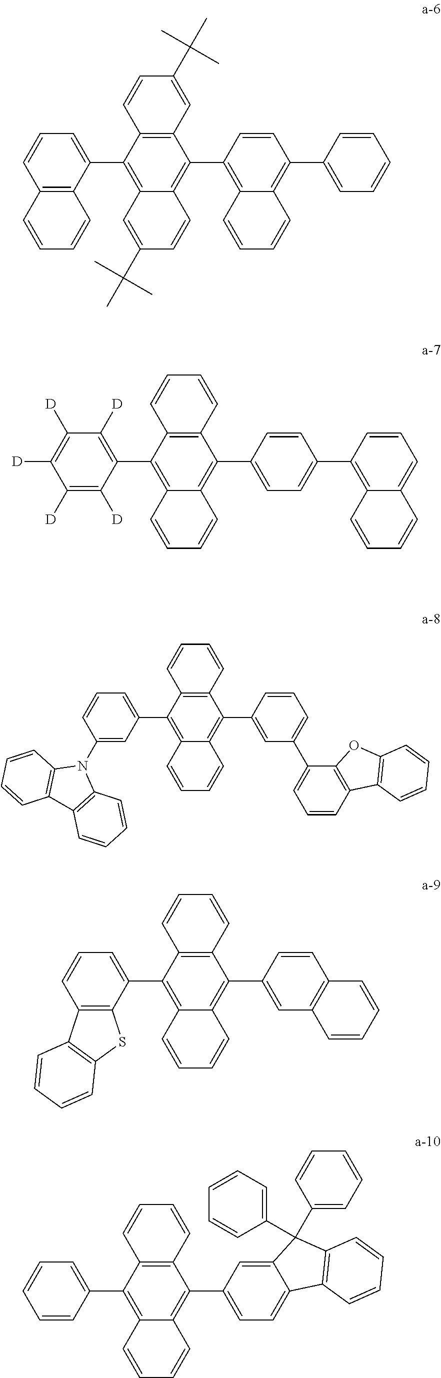

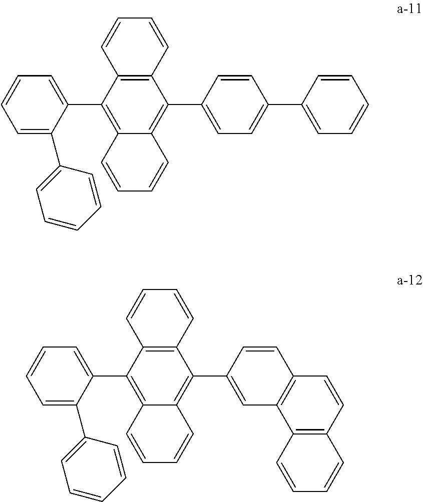

The emission layer EML may include a fluorescent material including any one selected from the group of spiro-DPVBi, 2,2′,7,7′-tetrakis(biphenyl-4-yl)-9,9′-spirobifluorene(spiro-sexiphenyl) (spiro-6P), distyryl-benzene (DSB), distyryl-arylene (DSA), polyfluorene (PFO)-based polymer and poly(p-phenylene vinylene) (PPV)-based polymer, for example.

The emission layer EML may further include a dopant, and the dopant may be a suitable material. For example, styryl derivatives (for example, 1,4-bis[2-(3-N-ethylcarbazolyl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi)), perylene and the derivatives thereof (for example, 2,5,8,11-tetra-t-butylperylene (TBPe)), pyrene and the derivatives thereof (for example, 1,1-dipyrene, 1,4-dipyrenylbenzene, 1,4-bis(N,N-diphenylamino)pyrene, 1,6-bis(N,N-diphenylamino)pyrene), 2,5,8,11-tetra-t-butylperylene (TBP), 1,3,5-tris(N-phenylbenzimidazole-2-yl)benzene) (TPBi), etc., may be used as a dopant.

The emission layer EML may include, for example, tris(8-hydroxyquinolino)aluminum (Alq₃), 4,4′-bis(N-carbazolyl)-1,1′-biphenyl (CBP), poly(N-vinylcarbazole) (PVK), 9,10-di(naphthalen-2-yl)anthracene (ADN), 4,4′,4″-tris(carbazol-9-yl)-triphenylamine (TCTA), 1,3,5-tris(N-phenylbenzimidazol-2-yl)benzene (TPBi), 3-tert-butyl-9,10-di(naphth-2-yl)anthracene (TBADN), distyrylarylene (DSA), 4,4′-bis(9-carbazolyl)-2,2′-dimethylbiphenyl (CDBP), 2-methyl-9,10-bis(naphthalen-2-yl)anthracene (MADN), bis[2-(diphenylphosphino)phenyl]ether oxide (DPEPO), hexaphenyl cyclotriphosphazene (CP1), 1,4-bis(triphenylsilyl)benzene (UGH2), hexaphenylcyclotrisiloxane (DPSiO₃), octaphenylcyclotetrasiloxane (DPSiO₄), 2,8-bis(diphenylphosphoryl)dibenzofuran (PPF), etc.

The electron transport region ETR is provided on the emission layer EML. The electron transport region ETR may include at least one of a hole blocking layer HBL, an electron transport layer ETL or an electron injection layer EIL, for example.

The electron transport region ETR may have a single layer formed using a single material, a single layer formed using a plurality of different materials, or a multilayer structure having a plurality of layers formed using a plurality of different materials.

For example, the electron transport region ETR may have a single layer structure of an electron injection layer EIL or an electron transport layer ETL, or a single layer structure formed using an electron injection material and an electron transport material. In addition, the electron transport region ETR may have a single layer structure having a plurality of different materials, or a laminated structure of electron transport layer ETL/electron injection layer EIL, or hole blocking layer HBL/electron transport layer ETL/electron injection layer EIL, laminated in order from the emission layer EML, without limitation. The thickness of the electron transport region ETR may be, for example, from about 100 Å to about 1,500 Å.

The electron transport region ETR may be formed using various methods such as a vacuum deposition method, a spin coating method, a cast method, a Langmuir-Blodgett (LB) method, an inkjet printing method, a laser printing method, and a laser induced thermal imaging (LITI) method.

In case the electron transport region ETR includes the electron transport layer ETL, the electron transport region ETR may include tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, bis[2-(diphenylphosphino)phenyl] ether oxide (DPEPO), 2-(4-(N-phenylbenzoimidazolyl-1-ylphenyl)-9,10-dinaphthylanthracene, 1,3,5-tri(1-phenyl-1H-benzo[d]imidazol-2-yl)phenyl (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-Biphenyl-4-olato)aluminum (BAlq), beryllium bis(benzoquinolin-10-olate) (Bebq₂), 9,10-di(naphthalen-2-yl)anthracene (ADN), or a mixture thereof, for example. The thickness of the electron transport layer ETL may be from about 100 Å to about 1,000 Å, for example, from about 150 Å to about 500 Å. If the thickness of the electron transport layer ETL satisfies the above-described range, satisfactory electron transport properties may be obtained without substantial increase of a driving voltage.

When the electron transport region ETR includes the electron injection layer EIL, the electron transport region ETR may use LiF, lithium quinolate (LIQ), Li₂O, BaO, NaCl, CsF, a metal in lanthanides such as Yb, or a metal halide such as RbCl and RbI, without limitation. The electron injection layer EIL also may be formed using a mixture material of an electron transport material and an insulating organo metal salt. The organo metal salt may be a material having an energy band gap of about 4 eV or more. The organo metal salt may include, for example, a metal acetate, a metal benzoate, a metal acetoacetate, a metal acetylacetonate, or a metal stearate. The thickness of the electron injection layer EIL may be from about 1 Å to about 100 Å, for example, from about 3 Å to about 90 Å. In case the thickness of the electron injection layer EIL satisfies the above described range, satisfactory electron injection properties may be obtained without inducing the substantial increase of a driving voltage.

The electron transport region ETR may include a hole blocking layer HBL, as described above. The hole blocking layer HBL may include, for example, 2,9-dimethyl-4,7-diphenyl-1,10-phenanthroline (BCP), 4,7-diphenyl-1,10-phenanthroline (Bphen), or bis[2-(diphenylphosphino)phenyl]ether oxide (DPEPO), etc.

The second electrode EL2 is on the electron transport region ETR. The second electrode EL2 may be a common electrode or a cathode. The second electrode EL2 may be a transmissive electrode, a transflective electrode or a reflective electrode. In case the second electrode EL2 is the transmissive electrode, the second electrode EL2 may be formed using transparent metal oxides, for example, ITO, IZO, ZnO, ITZO, etc.

In case the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, a compound thereof, or a mixture thereof (for example, a mixture of Ag and Mg). The second electrode EL2 may have a multilayer structure including a reflective layer or a transflective layer formed using the above-described materials and a transparent conductive layer formed using ITO, IZO, ZnO, ITZO, etc.

The second electrode EL2 may be connected with an auxiliary electrode. In case the second electrode EL2 is connected with the auxiliary electrode, the resistance of the second electrode EL2 may decrease.

In the organic electroluminescence device 10, according to the application of a voltage to each of the first electrode EL1 and the second electrode EL2, holes injected from the first electrode EL1 may move via the hole transport region HTR to the emission layer EML, and electrons injected from the second electrode EL2 may move via the electron transport region ETR to the emission layer EML. The electrons and the holes are recombined in the emission layer EML to generate excitons, and light may be emitted via the transition of the excitons from an excited state to a ground state.

In case the organic electroluminescence device 10 is a top emission type, the first electrode EL1 may be a reflective electrode, and the second electrode EL2 may be a transmissive electrode or a transflective electrode. In case the organic electroluminescence device 10 is a bottom emission type, the first electrode EL1 may be a transmissive electrode or a transflective electrode, and the second electrode EL2 may be a reflective electrode.

The organic electroluminescence device 10 according to an example embodiment includes the monoamine compound represented by Formula 1, thereby securing high efficiency and a long device life, as well as a decreased driving voltage.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### SYNTHESIS EXAMPLES

The monoamine compound according to an example embodiment may be synthesized, for example, as follows.

1. Synthesis of Compound A4

Compound A4, a monoamine compound according to an example embodiment may be synthesized, for example, as follows.

(Synthesis of Intermediate IM-1)

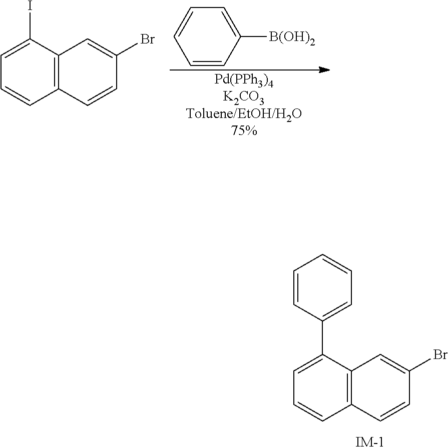

Under an Ar atmosphere, 7-bromo-1-iodonaphthalene (25.00 g, 75.1 mmol), phenylboronic acid (10.07 g, 1.1 equiv., 82.6 mmol), K₂CO₃ (31.13 g, 3.0 equiv., 225.2 mmol), Pd(PPh₃)₄ (4.34 g, 0.05 eq., 3.8 mmol), and a mixture solution of toluene/EtOH/H₂O (4/2/1) (525 mL) were added in order to an 1 L three-neck flask, and the mixture was stirred and heated at about 80° C. After cooling in air to room temperature, the reaction solution was extracted with toluene. After removing aqueous layer, organic layer was washed with saturated brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-1 (15.95 g, yield 75%). Intermediate IM-1 was identified by measuring FAB-MS in which a molecular ion peak was observed at mass m/z=283.

(Synthesis of Intermediate IM-2)

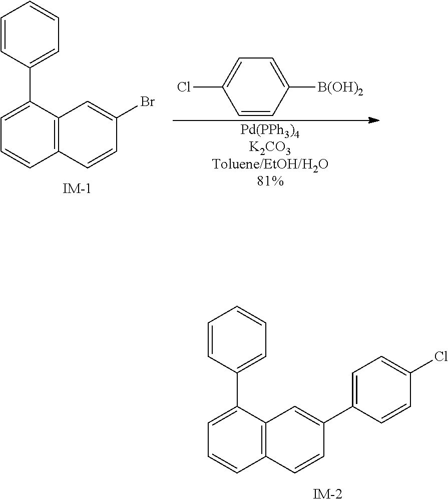

Under an Ar atmosphere, IM-1 (13.00 g, 45.9 mmol), 4-chlorophenylboronic acid (7.90 g, 1.1 equiv., 50.5 mmol), K₂CO₃ (19.04 g, 3.0 equiv., 60.7 mmol), Pd(PPh₃)₄ (2.65 g, 0.05 eq., 2.3 mmol), and a mixture solution of toluene/EtOH/H₂O (4/2/1) (321 mL) were added in order to an 1 L three-neck flask, and the mixture was stirred and heated at about 80° C. After cooling in air to room temperature, the reaction solution was extracted with toluene. After removing aqueous layer, organic layer was washed with saturated brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-2 (11.71 g, yield 81%). Intermediate IM-2 was identified by measuring FAB-MS in which a molecular ion peak was observed at mass m/z=314.

(Synthesis of Intermediate IM-3)

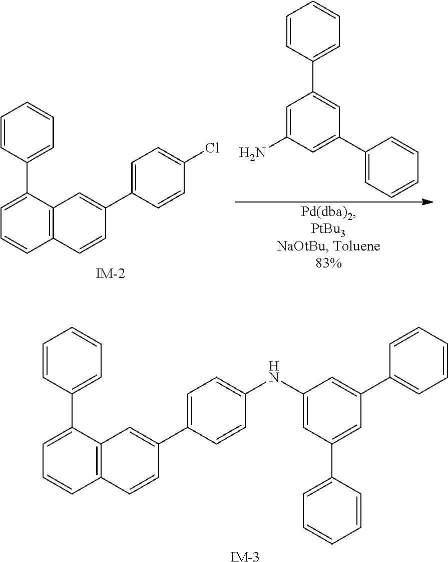

Under an Ar atmosphere, IM-2 (10.00 g, 31.8 mmol), Pd(dba)₂ (0.55 g, 0.03 equiv., 1.0 mmol), NaOtBu (3.05 g, 1.0 equiv., 31.8 mmol), toluene (159 mL), 3,5-diphenylaniline (8.57 g, 1.1 equiv., 34.9 mmol) and tBu₃P (0.64 g, 0.1 equiv., 3.2 mmol) were added in order to a 300 mL three-neck flask, and the mixture was stirred and heated to reflux. After cooling in air to room temperature, water was added to the reaction solvent and an organic layer was separated and taken. Toluene was added to the remaining aqueous layer, followed by extraction of the aqueous layer to obtain another organic layer. Organic layers were combined and washed with brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-3 (13.81 g, yield 83%). Intermediate IM-3 was identified by measuring FAB-MS in which a molecular ion peak was observed at mass m/z=523.

(Synthesis of Compound A4)

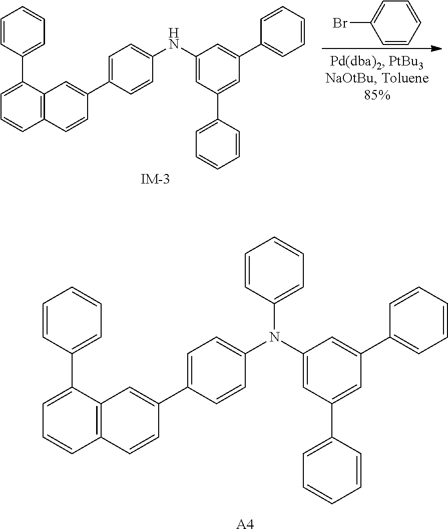

Under an Ar atmosphere, IM-3 (8.00 g, 15.3 mmol), Pd(dba)₂ (0.26 g, 0.03 equiv., 0.5 mmol), NaOtBu (2.94 g, 2.0 equiv., 30.6 mmol), toluene (76 mL), bromobenzene (2.64 g, 1.1 equiv., 16.8 mmol) and tBu₃P (0.31 g, 0.1 equiv., 1.5 mmol) were added in order to a 300 mL three-neck flask, and the mixture was stirred and heated to reflux. After cooling in air to room temperature, water was added to the reaction solvent and an organic layer was separated and taken. Toluene was added to the remaining aqueous layer, followed by extraction of the aqueous layer to obtain another organic layer. Organic layers were combined and washed with brine, and then dried over MgSO₄. MgSO₄ was filtered out and organic layer was concentrated. The crude product thus obtained was purified by silica gel column chromatography (using a mixture of hexane and toluene as developing solvent) to obtain Compound A4 (7.79 g, yield 85%).

Compound A4 was identified by measuring FAB-MS in which a molecular ion peak was observed at mass m/z=599.

2. Synthesis of Compound A17

Compound A17, a monoamine compound according to an example embodiment may be synthesized, for example, as follows.

(Synthesis of Intermediate IM-4)

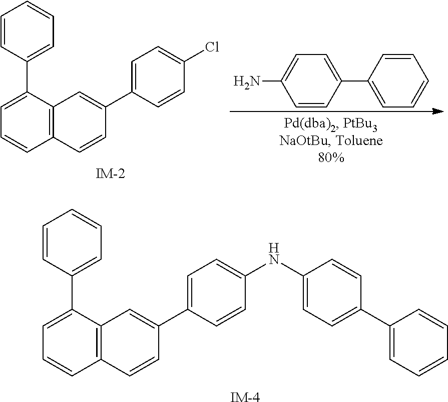

Under an Ar atmosphere, IM-2 (10.00 g, 31.8 mmol), Pd(dba)₂ (0.55 g, 0.03 equiv., 1.0 mmol), NaOtBu (3.05 g, 1.0 equiv., 31.8 mmol), toluene (159 mL), p-biphenylamine (5.91 g, 1.1 equiv., 34.9 mmol) and tBu₃P (0.64 g, 0.1 equiv., 3.2 mmol) were added in order to a 300 mL three-neck flask, and the mixture was stirred and heated to reflux. After cooling in air to room temperature, water was added to the reaction solvent and an organic layer was separated and taken. Toluene was added to the remaining aqueous layer, followed by extraction of the aqueous layer to obtain another organic layer. Organic layers were combined and washed with brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-4 (11.37 g, yield 80%). Intermediate IM-4 was identified by measuring FAB-MS in which a molecular ion peak was observed at mass m/z=447.

(Synthesis of Compound A17)

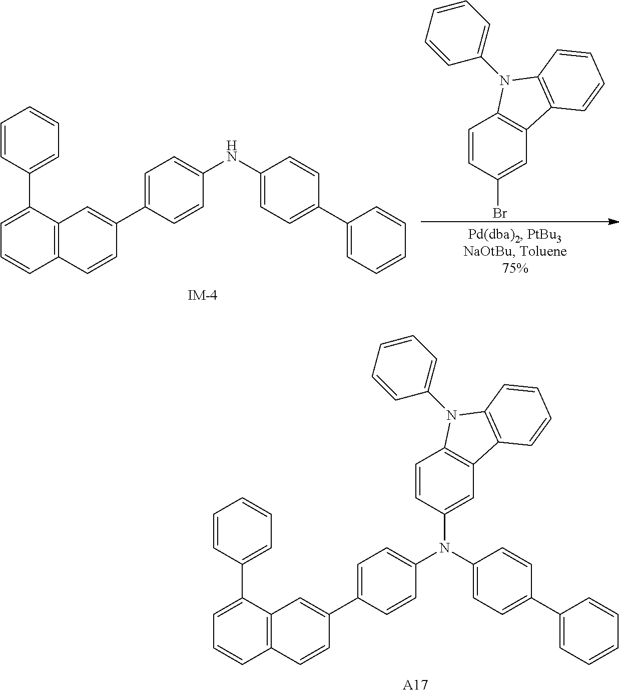

Under an Ar atmosphere, IM-4 (8.00 g, 17.9 mmol), Pd(dba)₂ (0.31 g, 0.03 equiv., 0.5 mmol), NaOtBu (3.44 g, 2.0 equiv., 35.7 mmol), toluene (89 mL), 3-bromo-9-phenyl-9H-carbazole (6.33 g, 1.1 equiv., 19.7 mmol) and tBu₃P (0.36 g, 0.1 equiv., 1.8 mmol) were added in order to a 300 mL three-neck flask, and the mixture was stirred and heated to reflux. After cooling in air to room temperature, water was added to the reaction solvent and an organic layer was separated and taken. Toluene was added to the remaining aqueous layer, followed by extraction of the aqueous layer to obtain another organic layer. Organic layers were combined and washed with brine, and then dried over MgSO₄. MgSO₄ was filtered out and organic layer was concentrated. The crude product thus obtained was purified by silica gel column chromatography (using a mixture of hexane and toluene as developing solvent) to obtain Compound A17 (9.23 g, yield 75%). Compound A17 was identified by measuring FAB-MS in which a molecular ion peak was observed at mass m/z=688.

3. Synthesis of Compound B13

Compound B13, a monoamine compound according to an example embodiment may be synthesized, for example, as follows.

(Synthesis of Intermediate IM-5)

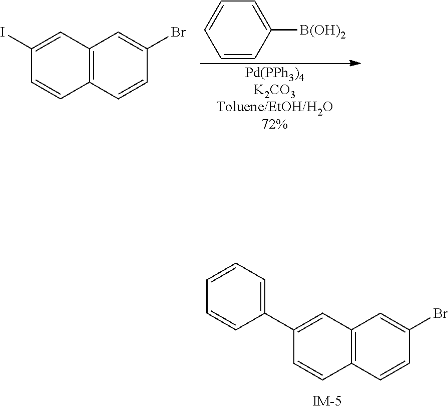

Under an Ar atmosphere, 7-bromo-2-iodonaphthalene (25.00 g, 75.1 mmol), phenylboronic acid (10.07 g, 1.1 equiv., 82.6 mmol), K₂CO₃ (31.13 g, 3.0 equiv., 225.2 mmol), Pd(PPh₃)₄ (4.34 g, 0.05 eq., 3.8 mmol), and a mixture solution of toluene/EtOH/H₂O (4/2/1) (525 mL) were added in order to an 1 L three-neck flask, and the mixture was stirred and heated at about 80° C. After cooling in air to room temperature, the reaction solution was extracted with toluene.

After removing aqueous layer, organic layer was washed with saturated brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-5 (15.31 g, yield 72%). Intermediate IM-5 was identified by measuring FAB-MS in which a molecular ion peak was observed at mass m/z=283.

(Synthesis of Intermediate IM-6)

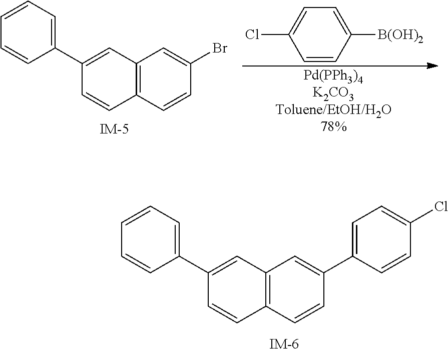

Under an Ar atmosphere, IM-5 (13.00 g, 45.9 mmol), 4-chlorophenylboronic acid (7.90 g, 1.1 equiv., 50.5 mmol), K₂CO₃ (19.04 g, 3.0 equiv., 60.7 mmol), Pd(PPh₃)₄ (2.65 g, 0.05 eq., 2.3 mmol), and a mixture solution of toluene/EtOH/H₂O (4/2/1) (321 mL) were added in order to an 1 L three-neck flask, and the mixture was stirred and heated at about 80° C. After cooling in air to room temperature, the reaction solution was extracted with toluene. After removing aqueous layer, organic layer was washed with saturated brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-6 (11.27 g, yield 78%). Intermediate IM-6 was identified by measuring FAB-MS in which a molecular ion peak was observed at mass m/z=314.

(Synthesis of Intermediate IM-7)

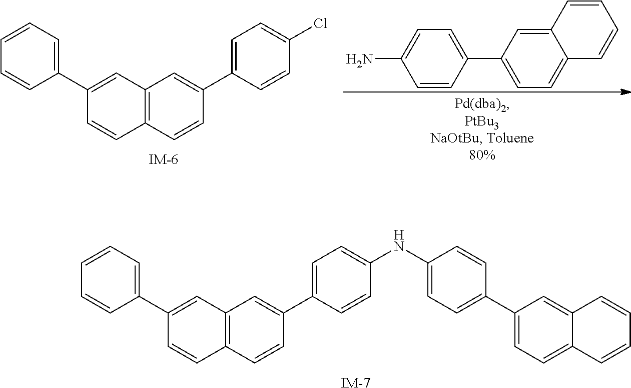

Under an Ar atmosphere, IM-6 (10.00 g, 31.8 mmol), Pd(dba)₂ (0.55 g, 0.03 equiv., 1.0 mmol), NaOtBu (3.05 g, 1.0 equiv., 31.8 mmol), toluene (159 mL), 4-(naphthalen-2-yl)aniline (7.66 g, 1.1 equiv., 34.9 mmol) and tBu₃P (0.64 g, 0.1 equiv., 3.2 mmol) were added in order to a 300 mL three-neck flask, and the mixture was stirred and heated to reflux. After cooling in air to room temperature, water was added to the reaction solvent and an organic layer was separated and taken. Toluene was added to the remaining aqueous layer, followed by extraction of the aqueous layer to obtain another organic layer. Organic layers were combined and washed with brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-7 (12.65 g, yield 80%). Intermediate IM-7 was identified by measuring FAB-MS in which a molecular ion peak was observed at mass m/z=497.

(Synthesis of Compound B13)

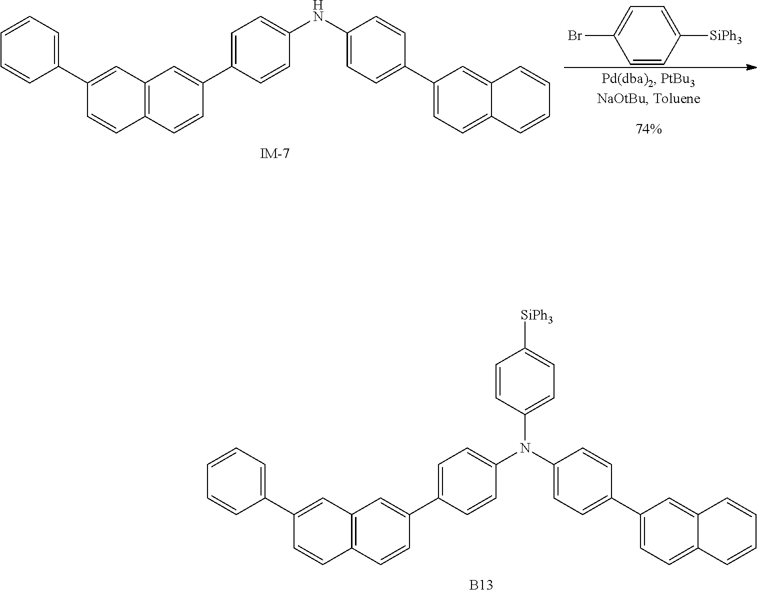

Under an Ar atmosphere, IM-7 (8.00 g, 16.1 mmol), Pd(dba)₂ (0.27 g, 0.03 equiv., 0.5 mmol), NaOtBu (3.09 g, 2.0 equiv., 32.2 mmol), toluene (80 mL), 1-bromo-4-triphenylsilylbenzene (7.35 g, 1.1 equiv., 17.7 mmol) and tBu₃P (0.33 g, 0.1 equiv., 1.6 mmol) were added in order to a 300 mL three-neck flask, and the mixture was stirred and heated to reflux. After cooling in air to room temperature, water was added to the reaction solvent and an organic layer was separated and taken. Toluene was added to the remaining aqueous layer, followed by extraction of the aqueous layer to obtain another organic layer. Organic layers were combined and washed with brine, and then dried over MgSO₄. MgSO₄ was filtered out and organic layer was concentrated. The crude product thus obtained was purified by silica gel column chromatography (using a mixture of hexane and toluene as developing solvent) to obtain Compound B13 (9.90 g, yield 74%). Compound B13 was identified by measuring FAB-MS in which a molecular ion peak was observed at mass m/z=832.

4. Synthesis of Compound B20

Compound B20, a monoamine compound according to an example embodiment may be synthesized, for example, as follows.

(Synthesis of Intermediate IM-8)

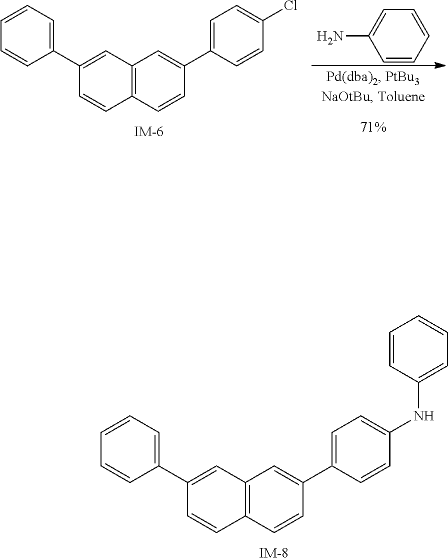

Under an Ar atmosphere, IM-6 (10.00 g, 31.8 mmol), Pd(dba)₂ (0.55 g, 0.03 equiv., 1.0 mmol), NaOtBu (3.05 g, 1.0 equiv., 31.8 mmol), toluene (159 mL), aniline (3.25 g, 1.1 equiv., 34.9 mmol) and tBu₃P (0.64 g, 0.1 equiv., 3.2 mmol) were added in order to a 300 mL three-neck flask, and the mixture was stirred and heated to reflux. After cooling in air to room temperature, water was added to the reaction solvent and an organic layer was separated and taken. Toluene was added to the remaining aqueous layer, followed by extraction of the aqueous layer to obtain another organic layer. Organic layers were combined and washed with brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-8 (8.38 g, yield 71%). Intermediate IM-8 was identified by measuring FAB-MS in which a molecular ion peak was observed at mass m/z=371.

(Synthesis of Compound B20)

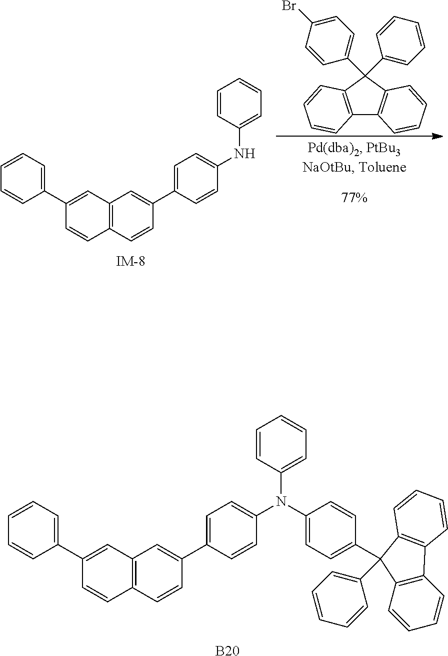

Under an Ar atmosphere, IM-8 (8.00 g, 21.5 mmol), Pd(dba)₂ (0.37 g, 0.03 equiv., 0.6 mmol), NaOtBu (4.14 g, 2.0 equiv., 43.1 mmol), toluene (108 mL), 9-(4-bromophenyl)-9-phenyl-9H-fluorene (9.41 g, 1.1 equiv., 23.7 mmol) and tBu₃P (0.44 g, 0.1 equiv., 2.1 mmol) were added in order to a 300 mL three-neck flask, and the mixture was stirred and heated to reflux. After cooling in air to room temperature, water was added to the reaction solvent and an organic layer was separated and taken. Toluene was added to the remaining aqueous layer, followed by extraction of the aqueous layer to obtain another organic layer. Organic layers were combined and washed with brine, and then dried over MgSO₄. MgSO₄ was filtered out and organic layer was concentrated. The crude product thus obtained was purified by silica gel column chromatography (using a mixture of hexane and toluene as developing solvent) to obtain Compound B20 (11.41 g, yield 77%). Compound B20 was identified by measuring FAB-MS in which a molecular ion peak was observed at mass m/z=687.

5. Synthesis of Compound B40

Compound B40, a monoamine compound according to an example embodiment may be synthesized, for example, as follows.

(Synthesis of Compound B40)

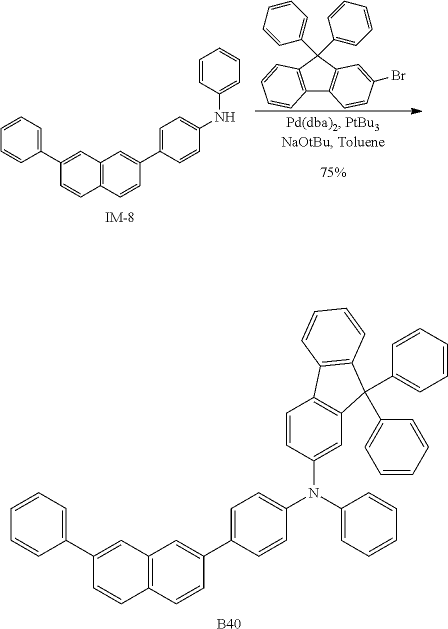

Under an Ar atmosphere, IM-8 (8.00 g, 21.5 mmol), Pd(dba)₂ (0.37 g, 0.03 equiv., 0.6 mmol), NaOtBu (4.14 g, 2.0 equiv., 43.1 mmol), toluene (108 mL), 2-bromo-9,9-diphenyl-9H-fluorene (9.41 g, 1.1 equiv., 23.7 mmol) and tBu₃P (0.44 g, 0.1 equiv., 2.1 mmol) were added in order to a 300 mL three-neck flask, and the mixture was stirred and heated to reflux. After cooling in air to room temperature, water was added to the reaction solvent and an organic layer was separated and taken. Toluene was added to the remaining aqueous layer, followed by extraction of the aqueous layer to obtain another organic layer. Organic layers were combined and washed with brine, and then dried over MgSO₄. MgSO₄ was filtered out and organic layer was concentrated. The crude product thus obtained was purified by silica gel column chromatography (using a mixture of hexane and toluene as developing solvent) to obtain Compound B40 (11.42 g, yield 75%). Compound B40 was identified by measuring FAB-MS in which a molecular ion peak was observed at mass m/z=687.

6. Synthesis of Compound C25

Compound C25, a monoamine compound according to an example embodiment may be synthesized, for example, as follows.

(Synthesis of Intermediate IM-9)

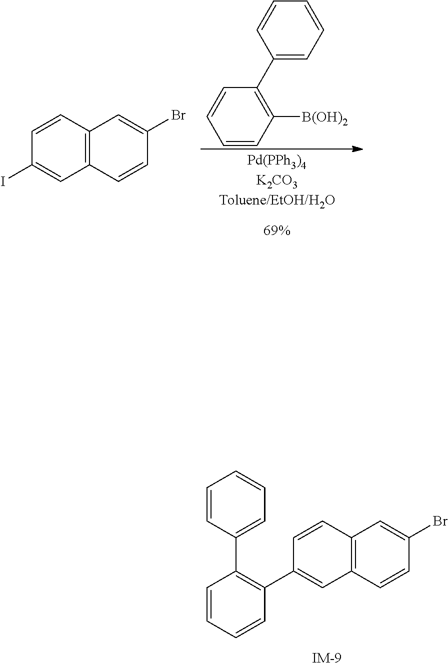

Under an Ar atmosphere, 2-bromo-6-iodonaphthalene (25.00 g, 75.1 mmol), 2-biphenylboronic acid (16.35 g, 1.1 equiv., 82.6 mmol), K₂CO₃ (31.13 g, 3.0 equiv., 225.2 mmol), Pd(PPh₃)₄ (4.34 g, 0.05 eq., 3.8 mmol), and a mixture solution of toluene/EtOH/H₂O (4/2/1) (525 mL) were added in order to an 1 L three-neck flask, and the mixture was stirred and heated at about 80° C. After cooling in air to room temperature, the reaction solution was extracted with toluene. After removing aqueous layer, organic layer was washed with saturated brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-9 (18.61 g, yield 69%). Intermediate IM-9 was identified by measuring FAB-MS in which a molecular ion peak was observed at mass m/z=359.

(Synthesis of Intermediate IM-10)

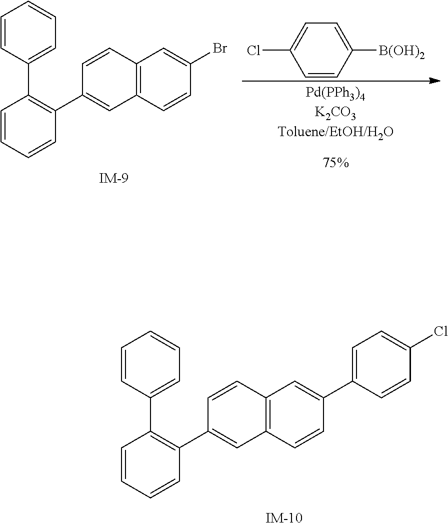

Under an Ar atmosphere, IM-9 (15.00 g, 41.8 mmol), 4-chlorophenylboronic acid (7.18 g, 1.1 equiv., 45.9 mmol), K₂CO₃ (17.31 g, 3.0 equiv., 125.3 mmol), Pd(PPh₃)₄ (2.41 g, 0.05 eq., 3.8 mmol), and a mixture solution of toluene/EtOH/H₂O (4/2/1) (525 mL) were added in order to an 1 L three-neck flask, and the mixture was stirred and heated at about 80° C. After cooling in air to room temperature, the reaction solution was extracted with toluene. After removing aqueous layer, organic layer was washed with saturated brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-10 (12.24 g, yield 75%). Intermediate IM-10 was identified by measuring FAB-MS in which a molecular ion peak was observed at mass m/z=390.

(Synthesis of Compound C25)

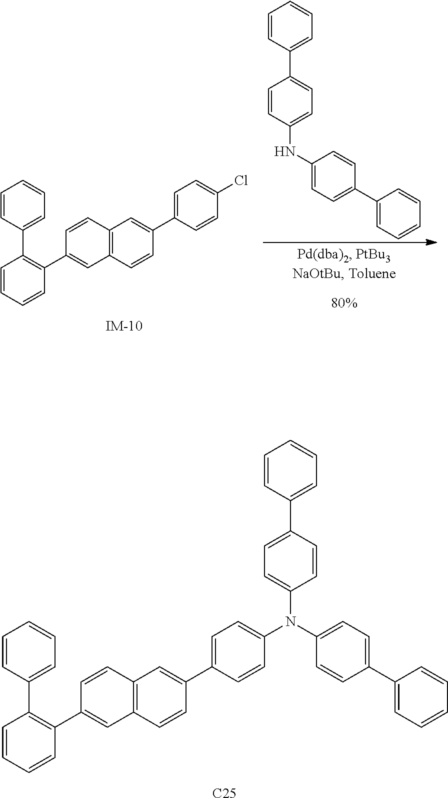

Under an Ar atmosphere, IM-10 (10.00 g, 25.6 mmol), Pd(dba)₂ (0.44 g, 0.03 equiv., 0.6 mmol), NaOtBu (4.92 g, 2.0 equiv., 51.2 mmol), toluene (128 mL), bis(4-biphenyl)amine (9.04 g, 1.1 equiv., 28.1 mmol) and tBu₃P (0.52 g, 0.1 equiv., 2.6 mmol) were added in order to a 300 mL three-neck flask, and the mixture was stirred and heated to reflux. After cooling in air to room temperature, water was added to the reaction solvent and an organic layer was separated and taken. Toluene was added to the remaining aqueous layer, followed by extraction of the aqueous layer to obtain another organic layer. Organic layers were combined and washed with brine, and then dried over MgSO₄. MgSO₄ was filtered out and organic layer was concentrated. The crude product thus obtained was purified by silica gel column chromatography (using a mixture of hexane and toluene as developing solvent) to obtain Compound C25 (13.83 g, yield 80%). Compound C25 was identified by measuring FAB-MS in which a molecular ion peak was observed at mass m/z=675.

7. Synthesis of Compound C51

Compound C51, a monoamine compound according to an example embodiment may be synthesized, for example, as follows.

(Synthesis of Intermediate IM-11)

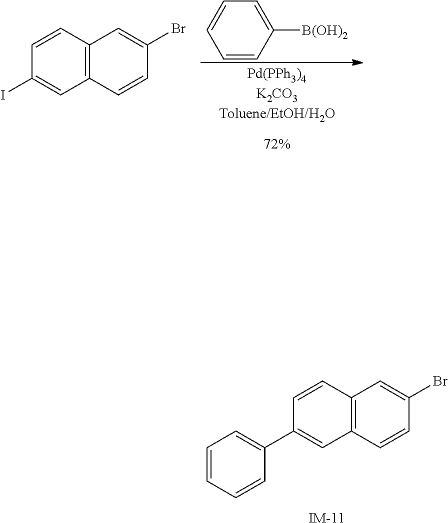

Under an Ar atmosphere, 2-bromo-6-iodonaphthalene (25.00 g, 75.1 mmol), phenylboronic acid (10.07 g, 1.1 equiv., 82.6 mmol), K₂CO₃ (31.13 g, 3.0 equiv., 225.2 mmol), Pd(PPh₃)₄ (4.34 g, 0.05 eq., 3.8 mmol), and a mixture solution of toluene/EtOH/H₂O (4/2/1) (525 mL) were added in order to an 1 L three-neck flask, and the mixture was stirred and heated at about 80° C. After cooling in air to room temperature, the reaction solution was extracted with toluene. After removing aqueous layer, organic layer was washed with saturated brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-11 (15.31 g, yield 72%). Intermediate IM-11 was identified by measuring FAB-MS in which a molecular ion peak was observed at mass m/z=283.

(Synthesis of Intermediate IM-12)

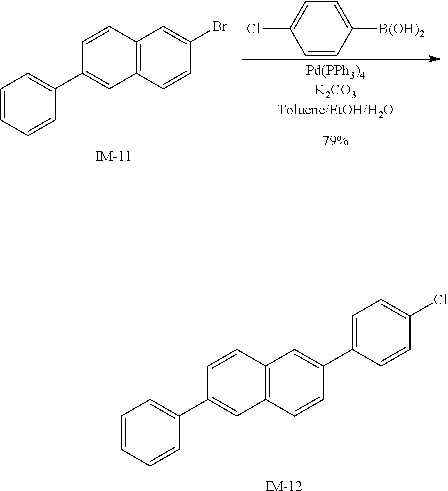

Under an Ar atmosphere, IM-11 (13.00 g, 45.9 mmol), 4-chlorophenylboronic acid (7.90 g, 1.1 equiv., 50.5 mmol), K₂CO₃ (19.04 g, 3.0 equiv., 60.7 mmol), Pd(PPh₃)₄ (2.65 g, 0.05 eq., 2.3 mmol), and a mixture solution of toluene/EtOH/H₂O (4/2/1) (321 mL) were added in order to an 1 L three-neck flask, and the mixture was stirred and heated at about 80° C. After cooling in air to room temperature, the reaction solution was extracted with toluene. After removing aqueous layer, organic layer was washed with saturated brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-12 (11.42 g, yield 79%).

Intermediate IM-12 was identified by measuring FAB-MS in which a molecular ion peak was observed at mass m/z=314.

(Synthesis of Compound C51)

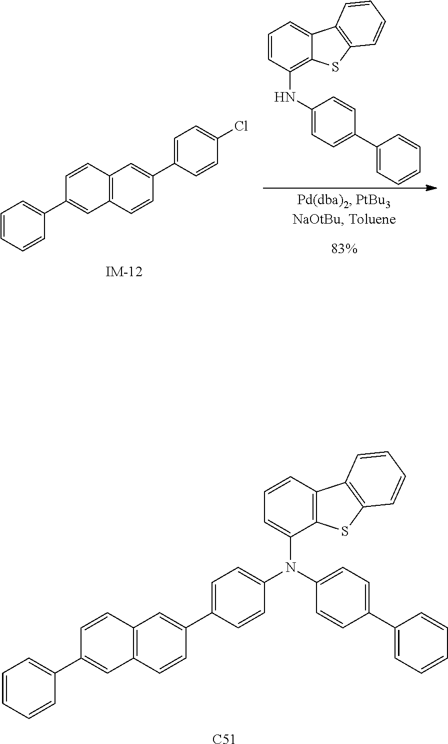

Under an Ar atmosphere, IM-12 (8.00 g, 25.4 mmol), Pd(dba)₂ (0.44 g, 0.03 equiv., 0.8 mmol), NaOtBu (4.88 g, 2.0 equiv., 50.8 mmol), toluene (128 mL), N-([1,1′-biphenyl]-4-yl)dibenzothiophen-4-amine (9.82 g, 1.1 equiv., 28.0 mmol) and tBu₃P (0.51 g, 0.1 equiv., 2.5 mmol) were added in order to a 300 mL three-neck flask, and the mixture was stirred and heated to reflux. After cooling in air to room temperature, water was added to the reaction solvent and an organic layer was separated and taken. Toluene was added to the remaining aqueous layer, followed by extraction of the aqueous layer to obtain another organic layer. Organic layers were combined and washed with brine, and then dried over MgSO₄. MgSO₄ was filtered out and organic layer was concentrated. The crude product thus obtained was purified by silica gel column chromatography (using a mixture of hexane and toluene as developing solvent) to obtain Compound C51 (13.28 g, yield 83%). Compound C51 was identified by measuring FAB-MS in which a molecular ion peak was observed at mass m/z=629.

8. Synthesis of Compound D12

Compound D12, a monoamine compound according to an example embodiment may be synthesized, for example, as follows.

(Synthesis of Intermediate IM-13)

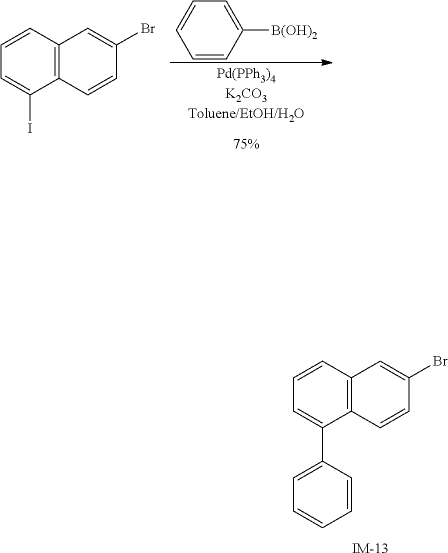

Under an Ar atmosphere, 2-bromo-5-iodonaphthalene (25.00 g, 75.1 mmol), phenylboronic acid (10.07 g, 1.1 equiv., 82.6 mmol), K₂CO₃ (31.13 g, 3.0 equiv., 225.2 mmol), Pd(PPh₃)₄ (4.34 g, 0.05 eq., 3.8 mmol), and a mixture solution of toluene/EtOH/H₂O (4/2/1) (525 mL) were added in order to an 1 L three-neck flask, and the mixture was stirred and heated at about 80° C. After cooling in air to room temperature, the reaction solution was extracted with toluene. After removing aqueous layer, organic layer was washed with saturated brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-13 (15.95 g, yield 75%).

Intermediate IM-13 was identified by measuring FAB-MS in which a molecular ion peak was observed at mass m/z=283.

(Synthesis of Intermediate IM-14)

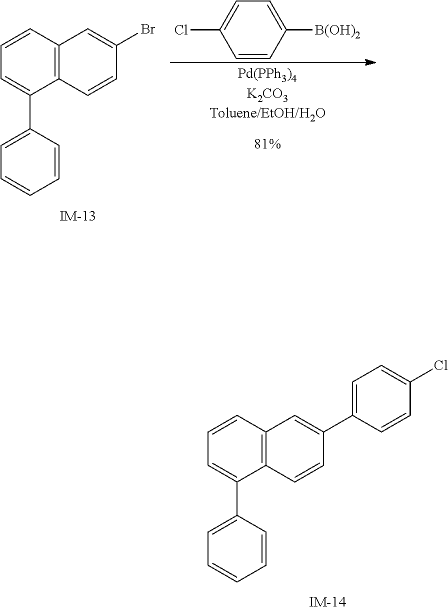

Under an Ar atmosphere, IM-13 (13.00 g, 45.9 mmol), 4-chlorophenylboronic acid (7.90 g, 1.1 equiv., 50.5 mmol), K₂CO₃ (19.04 g, 3.0 equiv., 60.7 mmol), Pd(PPh₃)₄ (2.65 g, 0.05 eq., 2.3 mmol), and a mixture solution of toluene/EtOH/H₂O (4/2/1) (321 mL) were added in order to an 1 L three-neck flask, and the mixture was stirred and heated at about 80° C. After cooling in air to room temperature, the reaction solution was extracted with toluene. After removing aqueous layer, organic layer was washed with saturated brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-14 (11.71 g, yield 81%). Intermediate IM-14 was identified by measuring FAB-MS in which a molecular ion peak was observed at mass m/z=314.

(Synthesis of Compound D12)

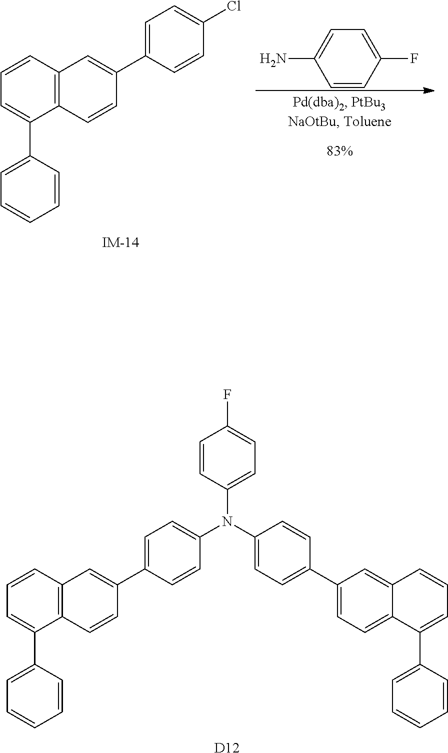

Under an Ar atmosphere, IM-14 (9.35 g, 2.2 equiv., 29.7 mmol), Pd(dba)₂ (0.23 g, 0.03 equiv., 0.4 mmol), NaOtBu (2.59 g, 2.0 equiv., 27.0 mmol), toluene (67 mL), 4-fluoroaniline (1.5 g, 13.5 mmol) and tBu₃P (0.27 g, 0.1 equiv., 1.3 mmol) were added in order to a 300 mL three-neck flask, and the mixture was stirred and heated to reflux. After cooling in air to room temperature, water was added to the reaction solvent and an organic layer was separated and taken. Toluene was added to the remaining aqueous layer, followed by extraction of the aqueous layer to obtain another organic layer. Organic layers were combined and washed with brine, and then dried over MgSO₄. MgSO₄ was filtered out and organic layer was concentrated. The crude product thus obtained was purified by silica gel column chromatography (using a mixture of hexane and toluene as developing solvent) to obtain Compound D12 (7.48 g, yield 83%).

Compound D12 was identified by measuring FAB-MS in which a molecular ion peak was observed at mass m/z=667.

9. Synthesis of Compound D22

Compound D22, a monoamine compound according to an example embodiment may be synthesized, for example, as follows.

(Synthesis of Compound D22)

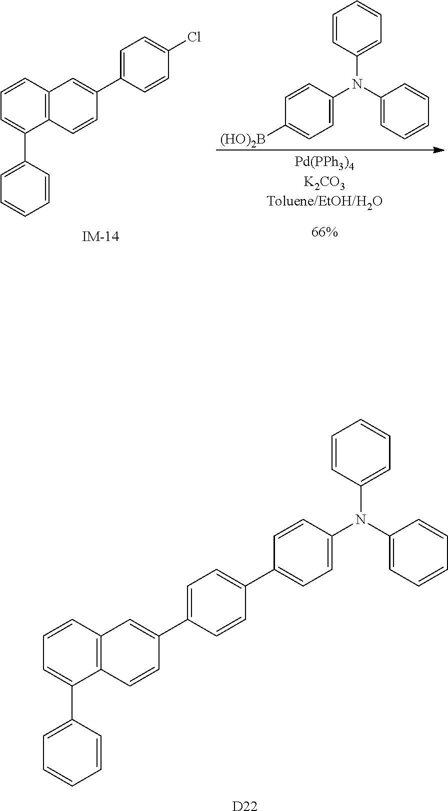

Under an Ar atmosphere, IM-14 (10.00 g, 31.8 mmol), (4-(diphenylamino)phenyl)boronic acid (10.10 g, 1.1 equiv., 34.9 mmol), K₂CO₃ (13.17 g, 3.0 equiv., 95.3 mmol), Pd(PPh₃)₄ (1.84 g, 0.05 eq., 1.6 mmol), and a mixture solution of toluene/EtOH/H₂O (4/2/1) (222 mL) were added in order to an 1 L three-neck flask, and the mixture was stirred and heated at about 80° C. After cooling in air to room temperature, the reaction solution was extracted with toluene. After removing aqueous layer, organic layer was washed with saturated brine, and then dried over MgSO₄. MgSO₄ was filtered out and organic layer was concentrated. The crude product thus obtained was purified by silica gel column chromatography (using a mixture of hexane and toluene as developing solvent) to obtain Compound D22 (10.98 g, yield 66%).

Compound D22 was identified by measuring FAB-MS in which a molecular ion peak was observed at mass m/z=523.

10. Synthesis of Compound E3

Compound E3, a monoamine compound according to an example embodiment may be synthesized, for example, as follows.

(Synthesis of Intermediate IM-15)

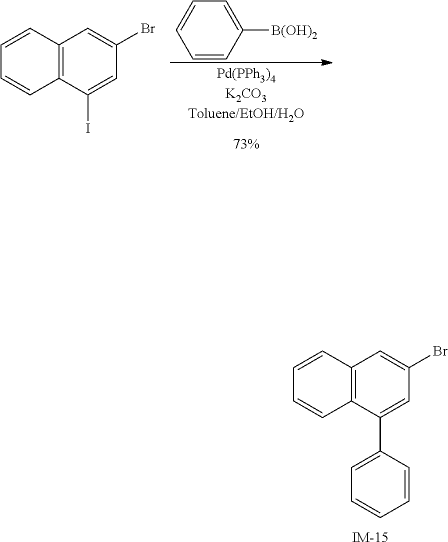

Under an Ar atmosphere, 3-bromo-1-iodonaphthalene (25.00 g, 75.1 mmol), phenylboronic acid (10.07 g, 1.1 equiv., 82.6 mmol), K₂CO₃ (31.13 g, 3.0 equiv., 225.2 mmol), Pd(PPh₃)₄ (4.34 g, 0.05 eq., 3.8 mmol), and a mixture solution of toluene/EtOH/H₂O (4/2/1) (525 mL) were added in order to an 1 L three-neck flask, and the mixture was stirred and heated at about 80° C. After cooling in air to room temperature, the reaction solution was extracted with toluene. After removing aqueous layer, organic layer was washed with saturated brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-15 (15.52 g, yield 73%).

Intermediate IM-15 was identified by measuring FAB-MS in which a molecular ion peak was observed at mass m/z=283.

(Synthesis of Intermediate IM-16)

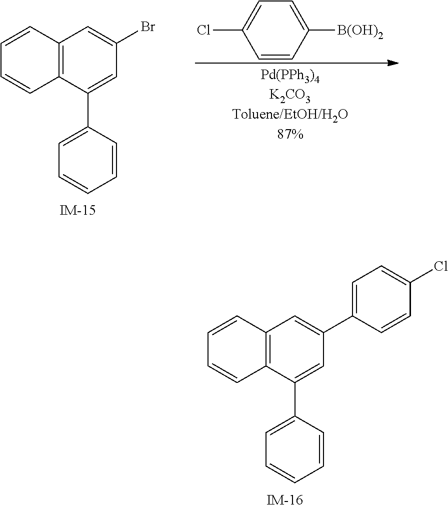

Under an Ar atmosphere, IM-15 (13.00 g, 45.9 mmol), 4-chlorophenylboronic acid (7.90 g, 1.1 equiv., 50.5 mmol), K₂CO₃ (19.04 g, 3.0 equiv., 60.7 mmol), Pd(PPh₃)₄ (2.65 g, 0.05 eq., 2.3 mmol), and a mixture solution of toluene/EtOH/H₂O (4/2/1) (321 mL) were added in order to an 1 L three-neck flask, and the mixture was stirred and heated at about 80° C. After cooling in air to room temperature, the reaction solution was extracted with toluene. After removing aqueous layer, organic layer was washed with saturated brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-16 (12.57 g, yield 87%).

Intermediate IM-16 was identified by measuring FAB-MS in which a molecular ion peak was observed at mass m/z=314.

(Synthesis of Intermediate IM-17)

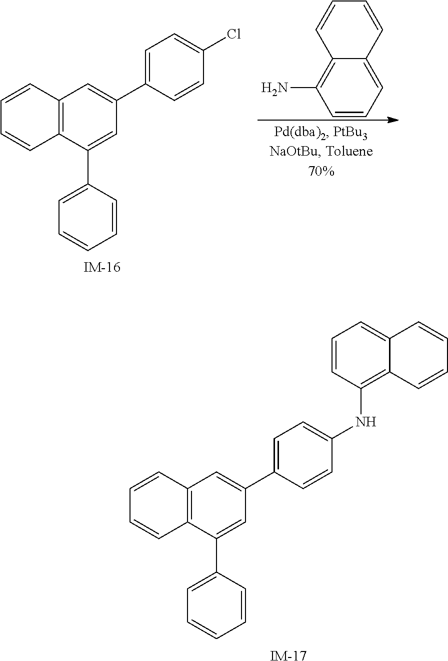

Under an Ar atmosphere, IM-16 (10.00 g, 31.8 mmol), Pd(dba)₂ (0.55 g, 0.03 equiv., 1.0 mmol), NaOtBu (3.05 g, 1.0 equiv., 31.8 mmol), toluene (159 mL), 1-naphthylamine (5.00 g, 1.1 equiv., 34.9 mmol) and tBu₃P (0.64 g, 0.1 equiv., 3.2 mmol) were added in order to a 300 mL three-neck flask, and the mixture was stirred and heated to reflux. After cooling in air to room temperature, water was added to the reaction solvent and an organic layer was separated and taken. Toluene was added to the remaining aqueous layer, followed by extraction of the aqueous layer to obtain another organic layer. Organic layers were combined and washed with brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-17 (9.37 g, yield 70%).

Intermediate IM-17 was identified by measuring FAB-MS in which a molecular ion peak was observed at mass m/z=421.

(Synthesis of Compound E3)

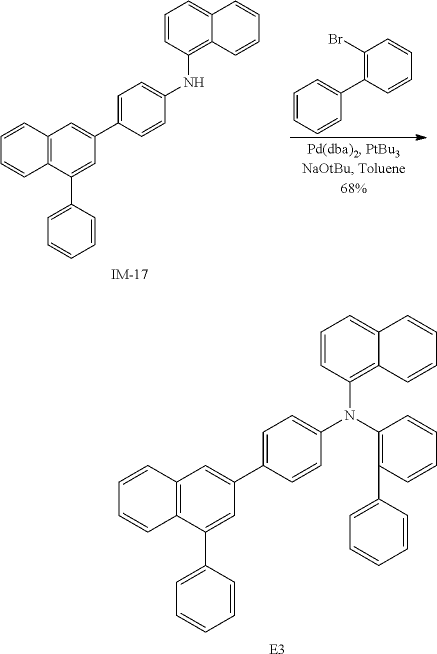

Under an Ar atmosphere, IM-17 (8.00 g, 19.0 mmol), Pd(dba)₂ (0.33 g, 0.03 equiv., 0.6 mmol), NaOtBu (3.65 g, 2.0 equiv., 38.0 mmol), toluene (95 mL), 2-bromobiphenyl (4.87 g, 1.1 equiv., 20.9 mmol) and tBu₃P (0.39 g, 0.1 equiv., 1.9 mmol) were added in order to a 300 mL three-neck flask, and the mixture was stirred and heated to reflux. After cooling in air to room temperature, water was added to the reaction solvent and an organic layer was separated and taken. Toluene was added to the remaining aqueous layer, followed by extraction of the aqueous layer to obtain another organic layer. Organic layers were combined and washed with brine, and then dried over MgSO₄. MgSO₄ was filtered out and organic layer was concentrated. The crude product thus obtained was purified by silica gel column chromatography (using a mixture of hexane and toluene as developing solvent) to obtain Compound E3 (7.40 g, yield 68%).

Compound E3 was identified by measuring FAB-MS in which a molecular ion peak was observed at mass m/z=573.

11. Synthesis of Compound E32

Compound E32, a monoamine compound according to an example embodiment may be synthesized, for example, as follows.

(Synthesis of Intermediate IM-18)

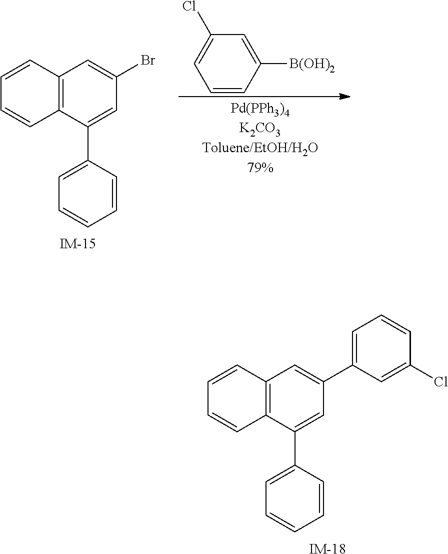

Under an Ar atmosphere, IM-15 (13.00 g, 45.9 mmol), 3-chlorophenylboronic acid (7.90 g, 1.1 equiv., 50.5 mmol), K₂CO₃ (19.04 g, 3.0 equiv., 60.7 mmol), Pd(PPh₃)₄ (2.65 g, 0.05 eq., 2.3 mmol), and a mixture solution of toluene/EtOH/H₂O (4/2/1) (321 mL) were added in order to an 1 L three-neck flask, and the mixture was stirred and heated at about 80° C. After cooling in air to room temperature, the reaction solution was extracted with toluene. After removing aqueous layer, organic layer was washed with saturated brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-18 (11.42 g, yield 79%).

Intermediate IM-18 was identified by measuring FAB-MS in which a molecular ion peak was observed at mass m/z=314.

(Synthesis of Compound E32)

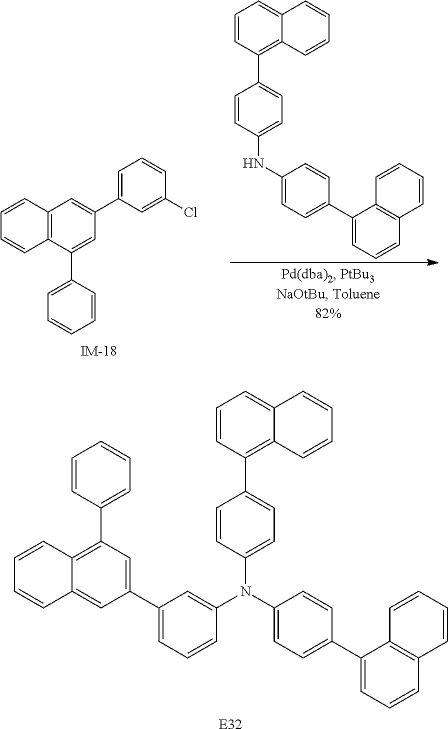

Under an Ar atmosphere, IM-18 (10.00 g, 31.8 mmol), Pd(dba)₂ (0.55 g, 0.03 equiv., 1.0 mmol), NaOtBu (6.11 g, 2.0 equiv., 63.5 mmol), toluene (158 mL), bis(4-(naphthalen-1-yl)phenyl)amine (14.73 g, 1.1 equiv., 34.9 mmol) and tBu₃P (0.64 g, 0.1 equiv., 3.2 mmol) were added in order to a 300 mL three-neck flask, and the mixture was stirred and heated to reflux. After cooling in air to room temperature, water was added to the reaction solvent and an organic layer was separated and taken. Toluene was added to the remaining aqueous layer, followed by extraction of the aqueous layer to obtain another organic layer. Organic layers were combined and washed with brine, and then dried over MgSO₄. MgSO₄ was filtered out and organic layer was concentrated. The crude product thus obtained was purified by silica gel column chromatography (using a mixture of hexane and toluene as developing solvent) to obtain Compound E32 (18.23 g, yield 82%).

Compound E32 was identified by measuring FAB-MS in which a molecular ion peak was observed at mass m/z=699.

12. Synthesis of Compound F46

Compound F46, a monoamine compound according to an example embodiment may be synthesized, for example, as follows.

(Synthesis of Intermediate IM-19)

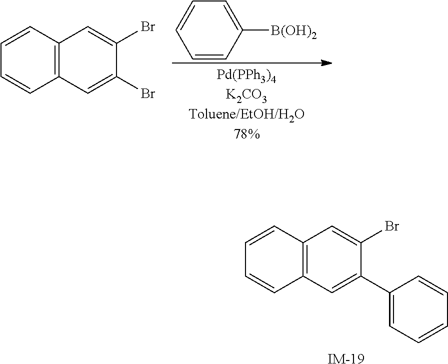

Under an Ar atmosphere, 2,3-dibromonaphthalene (25.00 g, 87.4 mmol), phenylboronic acid (11.73 g, 1.1 equiv., 96.2 mmol), K₂CO₃ (36.2 g, 3.0 equiv., 262.3 mmol), Pd(PPh₃)₄ (5.05 g, 0.05 eq., 3.4 mmol), and a mixture solution of toluene/EtOH/H₂O (4/2/1) (612 mL) were added in order to an 1 L three-neck flask, and the mixture was stirred and heated at about 80° C. After cooling in air to room temperature, the reaction solution was extracted with toluene. After removing aqueous layer, organic layer was washed with saturated brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-19 (19.31 g, yield 78%).

Intermediate IM-19 was identified by measuring FAB-MS in which a molecular ion peak was observed at mass m/z=283.

(Synthesis of Intermediate IM-20)

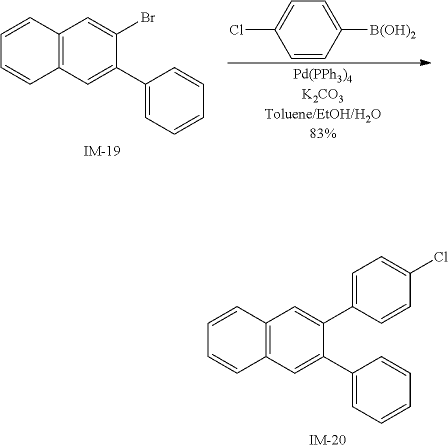

Under an Ar atmosphere, IM-19 (13.00 g, 45.9 mmol), 4-chlorophenylboronic acid (7.90 g, 1.1 equiv., 50.5 mmol), K₂CO₃ (19.04 g, 3.0 equiv., 60.7 mmol), Pd(PPh₃)₄ (2.65 g, 0.05 eq., 2.3 mmol), and a mixture solution of toluene/EtOH/H₂O (4/2/1) (321 mL) were added in order to an 1 L three-neck flask, and the mixture was stirred and heated at about 80° C. After cooling in air to room temperature, the reaction solution was extracted with toluene. After removing aqueous layer, organic layer was washed with saturated brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-20 (12.00 g, yield 83%).

Intermediate IM-20 was identified by measuring FAB-MS in which a molecular ion peak was observed at mass m/z=314.

(Synthesis of Intermediate IM-21)

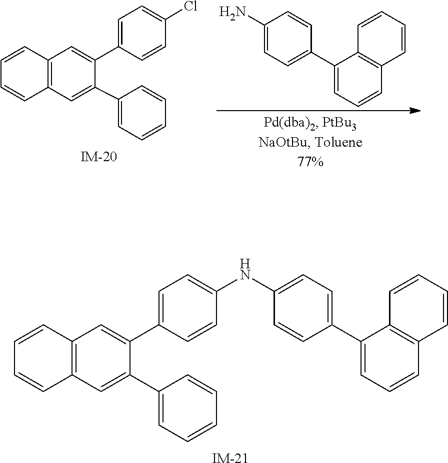

Under an Ar atmosphere, IM-20 (10.00 g, 31.8 mmol), Pd(dba)₂ (0.55 g, 0.03 equiv., 1.0 mmol), NaOtBu (3.05 g, 1.0 equiv., 31.8 mmol), toluene (159 mL), 4-(naphthalen-1-yl)aniline (7.66 g, 1.1 equiv., 34.9 mmol) and tBu₃P (0.64 g, 0.1 equiv., 3.2 mmol) were added in order to a 300 mL three-neck flask, and the mixture was stirred and heated to reflux. After cooling in air to room temperature, water was added to the reaction solvent and an organic layer was separated and taken. Toluene was added to the remaining aqueous layer, followed by extraction of the aqueous layer to obtain another organic layer. Organic layers were combined and washed with brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-21 (10.31 g, yield 77%).

Intermediate IM-21 was identified by measuring FAB-MS in which a molecular ion peak was observed at mass m/z=421.

(Synthesis of Compound F46)

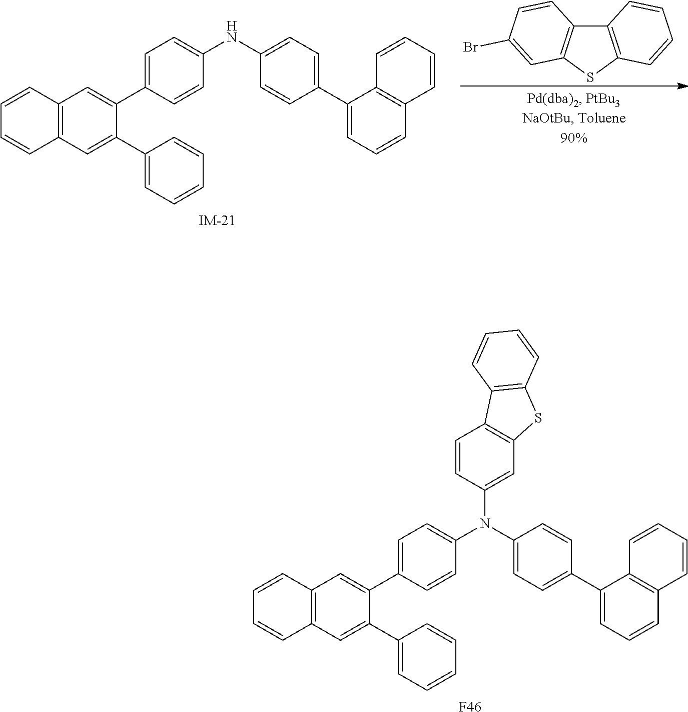

Under an Ar atmosphere, IM-21 (8.00 g, 19.0 mmol), Pd(dba)₂ (0.33 g, 0.03 equiv., 0.6 mmol), NaOtBu (3.65 g, 2.0 equiv., 38.0 mmol), toluene (95 mL), 3-bromo-dibenzothiophen (5.49 g, 1.1 equiv., 20.9 mmol) and tBu₃P (0.39 g, 0.1 equiv., 1.9 mmol) were added in order to a 300 mL three-neck flask, and the mixture was stirred and heated to reflux. After cooling in air to room temperature, water was added to the reaction solvent and an organic layer was separated and taken. Toluene was added to the remaining aqueous layer, followed by extraction of the aqueous layer to obtain another organic layer. Organic layers were combined and washed with brine, and then dried over MgSO₄. MgSO₄ was filtered out and organic layer was concentrated. The crude product thus obtained was purified by silica gel column chromatography (using a mixture of hexane and toluene as developing solvent) to obtain Compound F46 (11.61 g, yield 90%).

Compound F46 was identified by measuring FAB-MS in which a molecular ion peak was observed at mass m/z=679.

13. Synthesis of Compound F53

Compound F53, a monoamine compound according to an example embodiment may be synthesized, for example, as follows.

(Synthesis of Compound F53)

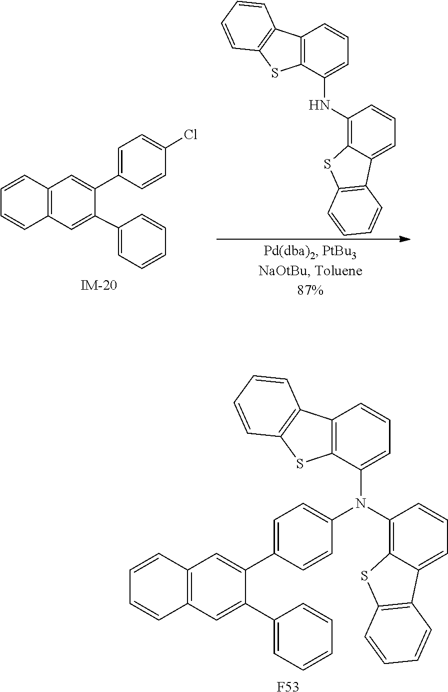

Under an Ar atmosphere, IM-20 (8.00 g, 23.4 mmol), Pd(dba)₂ (0.40 g, 0.03 equiv., 0.7 mmol), NaOtBu (4.50 g, 2.0 equiv., 46.8 mmol), toluene (117 mL), bis(dibenzothiophen-4-yl)amine (9.82 g, 1.1 equiv., 25.7 mmol) and tBu₃P (0.47 g, 0.1 equiv., 2.3 mmol) were added in order to a 300 mL three-neck flask, and the mixture was stirred and heated to reflux. After cooling in air to room temperature, water was added to the reaction solvent and an organic layer was separated and taken. Toluene was added to the remaining aqueous layer, followed by extraction of the aqueous layer to obtain another organic layer. Organic layers were combined and washed with brine, and then dried over MgSO₄. MgSO₄ was filtered out and organic layer was concentrated. The crude product thus obtained was purified by silica gel column chromatography (using a mixture of hexane and toluene as developing solvent) to obtain Compound F53 (13.44 g, yield 87%).

Compound F53 was identified by measuring FAB-MS in which a molecular ion peak was observed at mass m/z=659.

14. Synthesis of Compound G54

Compound G54, a monoamine compound according to an example embodiment may be synthesized, for example, as follows.

(Synthesis of Intermediate IM-22)

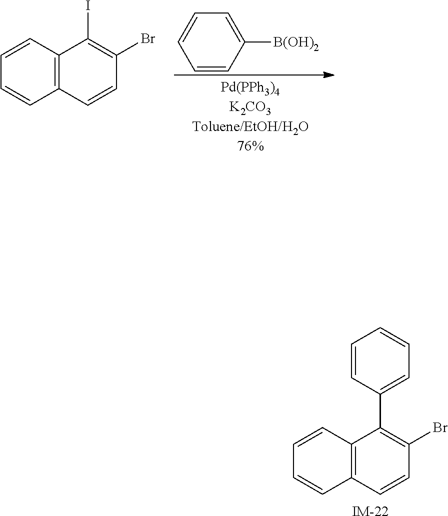

Under an Ar atmosphere, 2-bromo-1-iodo-naphthalene (25.00 g, 75.1 mmol), phenylboronic acid (10.07 g, 1.1 equiv., 82.6 mmol), K₂CO₃ (31.1 g, 3.0 equiv., 225.2 mmol), Pd(PPh₃)₄ (4.34 g, 0.05 eq., 3.8 mmol), and a mixture solution of toluene/EtOH/H₂O (4/2/1) (525 mL) were added in order to an 1 L three-neck flask, and the mixture was stirred and heated at about 80° C. After cooling in air to room temperature, the reaction solution was extracted with toluene. After removing aqueous layer, organic layer was washed with saturated brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-22 (16.16 g, yield 76%).

Intermediate IM-22 was identified by measuring FAB-MS in which a molecular ion peak was observed at mass m/z=283.

(Synthesis of Intermediate IM-23)

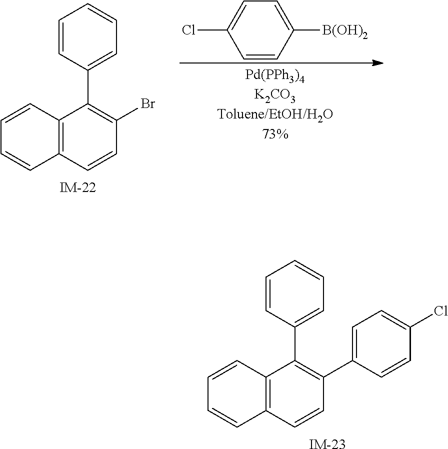

Under an Ar atmosphere, IM-22 (13.00 g, 45.9 mmol), 4-chlorophenylboronic acid (7.90 g, 1.1 equiv., 50.5 mmol), K₂CO₃ (19.04 g, 3.0 equiv., 60.7 mmol), Pd(PPh₃)₄ (2.65 g, 0.05 eq., 2.3 mmol), and a mixture solution of toluene/EtOH/H₂O (4/2/1) (321 mL) were added in order to an 1 L three-neck flask, and the mixture was stirred and heated at about 80° C. After cooling in air to room temperature, the reaction solution was extracted with toluene. After removing aqueous layer, organic layer was washed with saturated brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-23 (10.55 g, yield 73%).

Intermediate IM-23 was identified by measuring FAB-MS in which a molecular ion peak was observed at mass m/z=314.

(Synthesis of Compound G54)

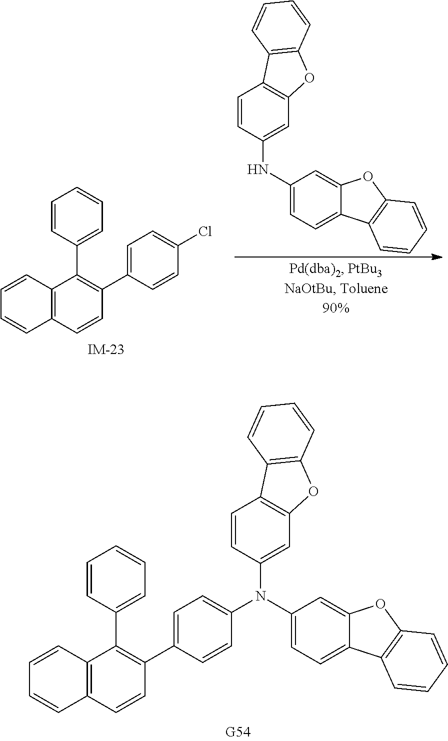

Under an Ar atmosphere, IM-23 (8.00 g, 25.4 mmol), Pd(dba)₂ (0.44 g, 0.03 equiv., 0.8 mmol), NaOtBu (4.88 g, 2.0 equiv., 50.8 mmol), toluene (127 mL), bis(dibenzofuran-3-yl)amine (9.77 g, 1.1 equiv., 28.0 mmol) and tBu₃P (0.51 g, 0.1 equiv., 2.5 mmol) were added in order to a 300 mL three-neck flask, and the mixture was stirred and heated to reflux. After cooling in air to room temperature, water was added to the reaction solvent and an organic layer was separated and taken. Toluene was added to the remaining aqueous layer, followed by extraction of the aqueous layer to obtain another organic layer. Organic layers were combined and washed with brine, and then dried over MgSO₄. MgSO₄ was filtered out and organic layer was concentrated. The crude product thus obtained was purified by silica gel column chromatography (using a mixture of hexane and toluene as developing solvent) to obtain Compound G54 (14.4 g, yield 90%).

Compound G54 was identified by measuring FAB-MS in which a molecular ion peak was observed at mass m/z=627.

15. Synthesis of Compound G58

Compound G58, a monoamine compound according to an example embodiment may be synthesized, for example, as follows.

(Synthesis of Intermediate IM-24)

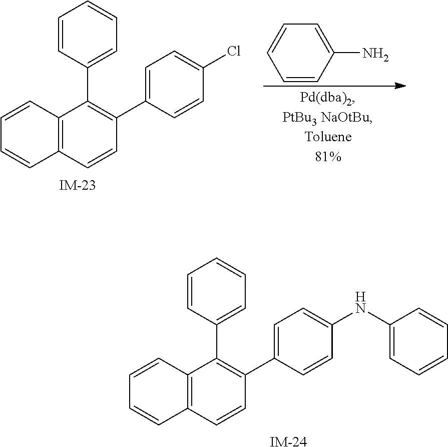

Under an Ar atmosphere, IM-23 (10.00 g, 31.8 mmol), Pd(dba)₂ (0.55 g, 0.03 equiv., 1.0 mmol), NaOtBu (3.05 g, 1.0 equiv., 31.8 mmol), toluene (159 mL), aniline (3.25 g, 1.1 equiv., 34.9 mmol) and tBu₃P (0.64 g, 0.1 equiv., 3.2 mmol) were added in order to a 300 mL three-neck flask, and the mixture was stirred and heated to reflux. After cooling in air to room temperature, water was added to the reaction solvent and an organic layer was separated and taken. Toluene was added to the remaining aqueous layer, followed by extraction of the aqueous layer to obtain another organic layer. Organic layers were combined and washed with brine, and then dried over MgSO₄. MgSO₄ was filtered out and organic layer was concentrated. The crude product thus obtained was purified by silica gel column chromatography (using a mixture of hexane and toluene as developing solvent) to obtain Intermediate IM-24 (9.56 g, yield 81%).

Intermediate IM-24 was identified by measuring FAB-MS in which a molecular ion peak was observed at mass m/z=371.

(Synthesis of Compound G58)

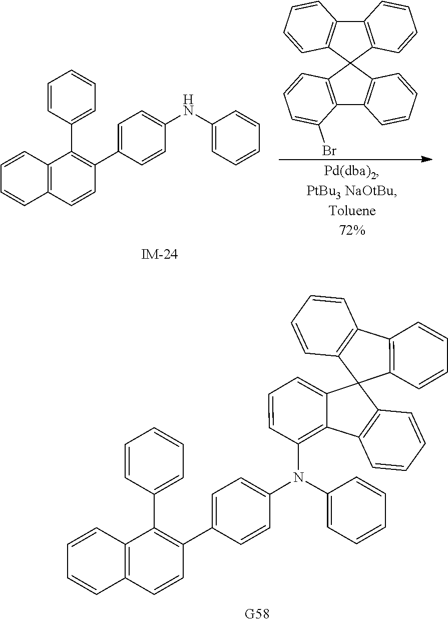

Under an Ar atmosphere, IM-24 (8.00 g, 21.5 mmol), Pd(dba)₂ (0.37 g, 0.03 equiv., 0.6 mmol), NaOtBu (4.14 g, 2.0 equiv., 43.1 mmol), toluene (108 mL), 4-bromo-9,9′-spirobifluorene (9.36 g, 1.1 equiv., 23.7 mmol) and tBu₃P (0.44 g, 0.1 equiv., 2.2 mmol) were added in order to a 300 mL three-neck flask, and the mixture was stirred and heated to reflux. After cooling in air to room temperature, water was added to the reaction solvent and an organic layer was separated and taken. Toluene was added to the remaining aqueous layer, followed by extraction of the aqueous layer to obtain another organic layer. Organic layers were combined and washed with brine, and then dried over MgSO₄. MgSO₄ was filtered out and organic layer was concentrated. The crude product thus obtained was purified by silica gel column chromatography (using a mixture of hexane and toluene as developing solvent) to obtain Compound G58 (10.63 g, yield 72%).

Compound G58 was identified by measuring FAB-MS in which a molecular ion peak was observed at mass m/z=685.

(Device Manufacturing Example)

Organic electroluminescence devices of Examples 1 to 15 were manufactured by using the above Compounds A4, A17, B13, B20, B40, C25, C51, D12, D22, E3, E32, F46, F53, G54, and G58 as an electron blocking material.

[Example Compounds]

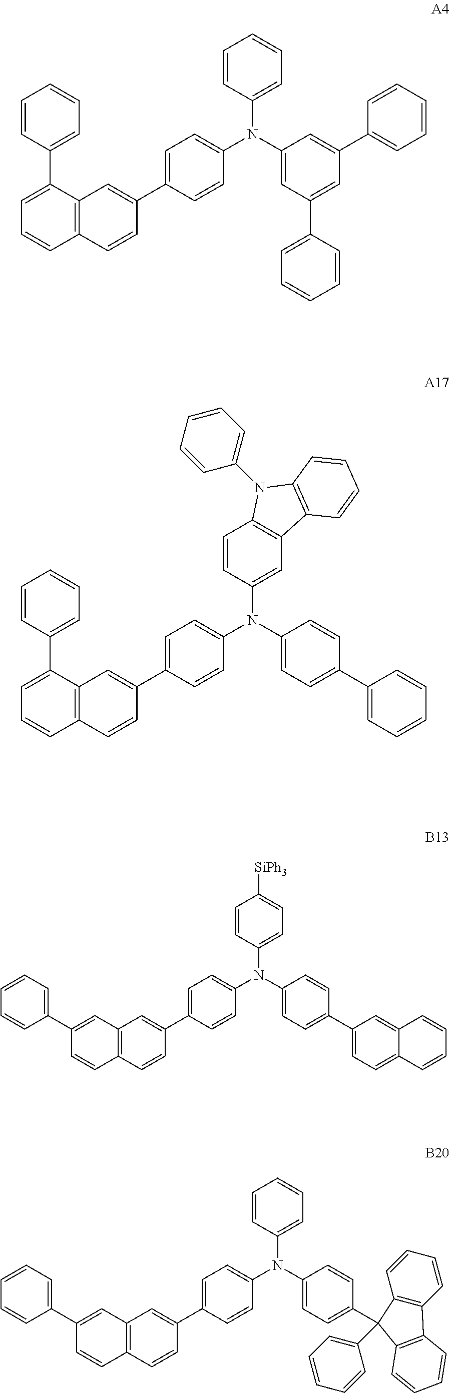

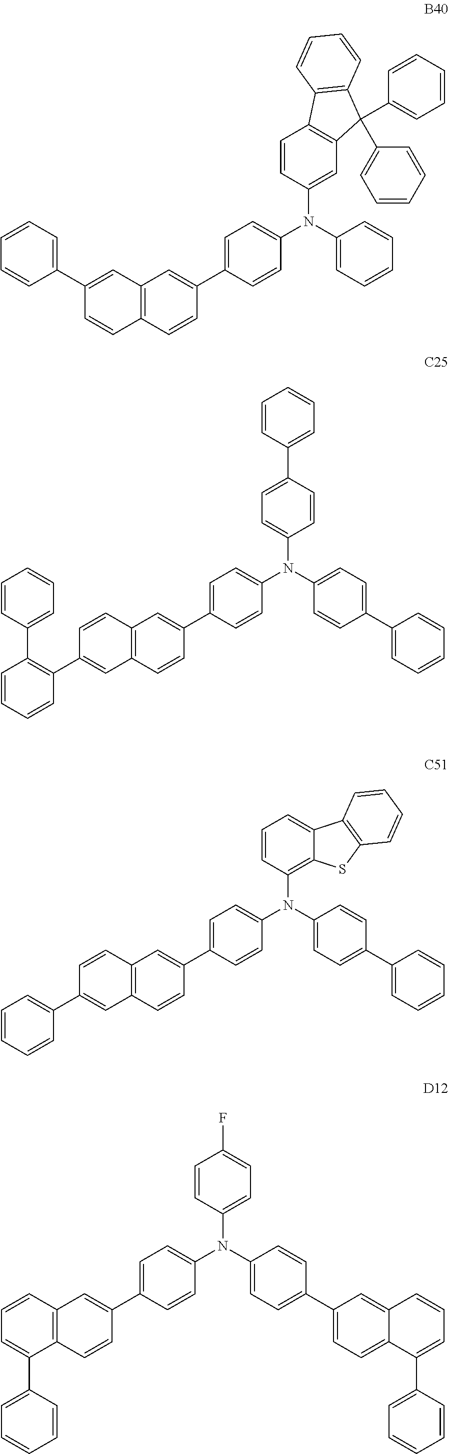 [SMILES: c1ccc(-c2ccc3ccc(-c4ccc(N(c5ccccc5)c5ccc6c(c5)C(c5ccccc5)(c5ccccc5)c5ccccc5-6)cc4)cc3c2)cc1 ; c1ccc(-c2ccc(N(c3ccc(-c4ccccc4)cc3)c3ccc(-c4ccc5cc(-c6ccccc6-c6ccccc6)ccc5c4)cc3)cc2)cc1 ; c1ccc(-c2ccc(N(c3ccc(-c4ccc5cc(-c6ccccc6)ccc5c4)cc3)c3cccc4c3sc3ccccc34)cc2)cc1 ; Fc1ccc(N(c2ccc(-c3ccc4c(-c5ccccc5)cccc4c3)cc2)c2ccc(-c3ccc4c(-c5ccccc5)cccc4c3)cc2)cc1]

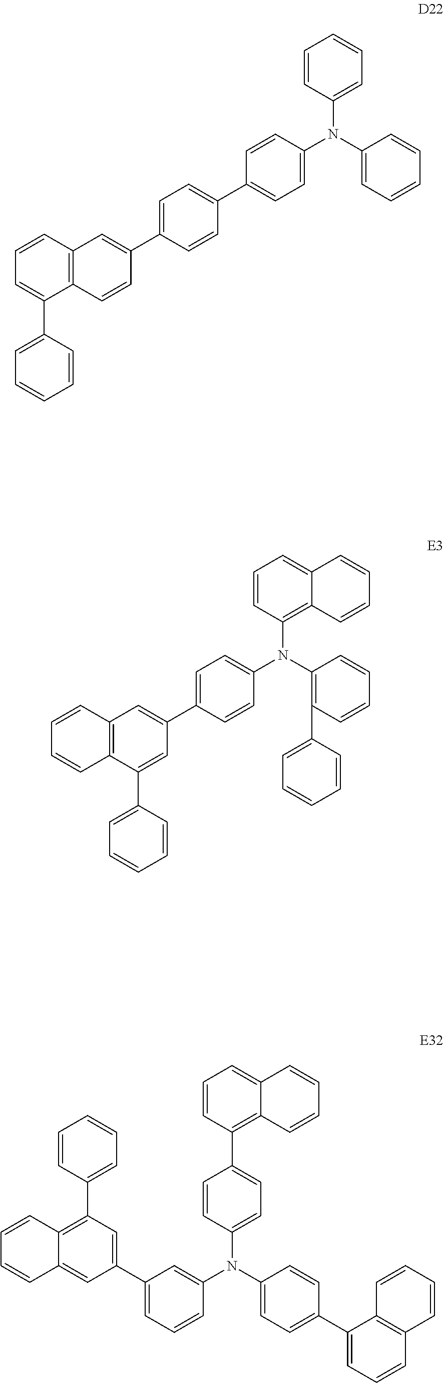 [SMILES: c1ccc(-c2cccc3cc(-c4ccc(-c5ccc(N(c6ccccc6)c6ccccc6)cc5)cc4)ccc23)cc1 ; c1ccc(-c2ccccc2N(c2ccc(-c3cc(-c4ccccc4)c4ccccc4c3)cc2)c2cccc3ccccc23)cc1 ; c1ccc(-c2cc(-c3cccc(N(c4ccc(-c5cccc6ccccc56)cc4)c4ccc(-c5cccc6ccccc56)cc4)c3)cc3ccccc23)cc1]

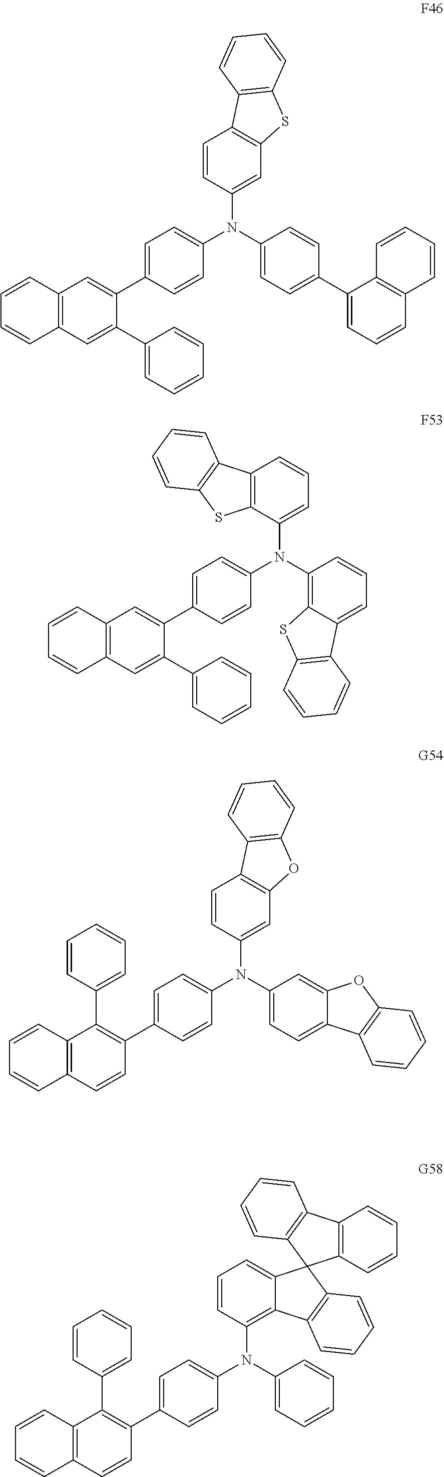

Organic electroluminescent devices of Comparative Examples 1 to 8 were manufactured by using the following Comparative Compounds R-1 to R-8.

[Comparative Compounds]

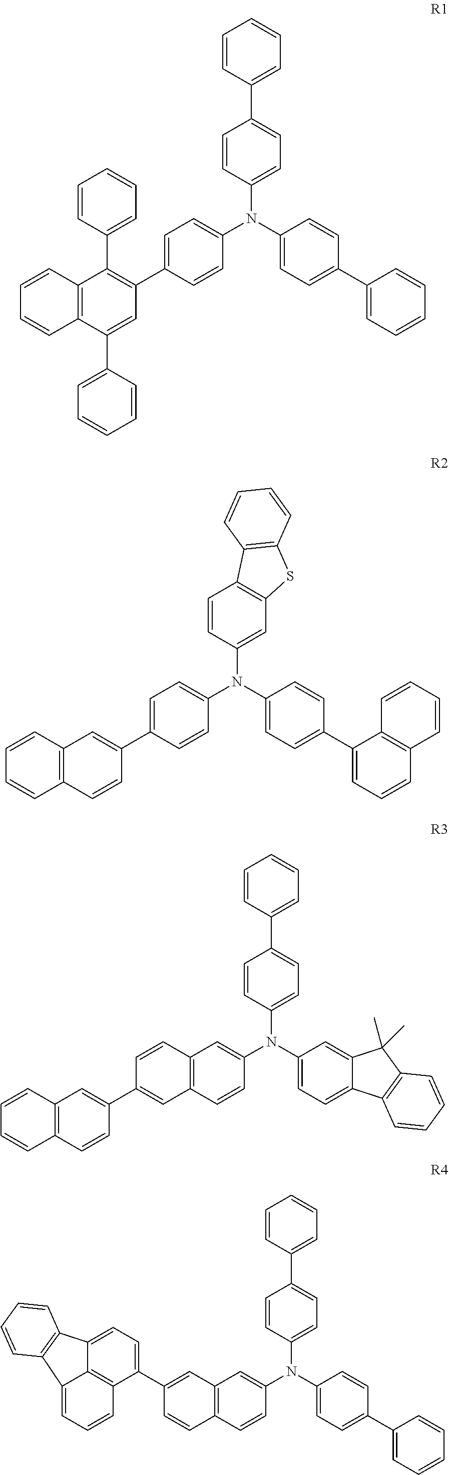 [SMILES: c1ccc(-c2ccc(N(c3ccc(-c4ccccc4)cc3)c3ccc(-c4cc(-c5ccccc5)c5ccccc5c4-c4ccccc4)cc3)cc2)cc1 ; c1ccc2cc(-c3ccc(N(c4ccc(-c5cccc6ccccc56)cc4)c4ccc5c(c4)sc4ccccc45)cc3)ccc2c1 ; CC1(C)c2ccccc2-c2ccc(N(c3ccc(-c4ccccc4)cc3)c3ccc4cc(-c5ccc6ccccc6c5)ccc4c3)cc21 ; c1ccc(-c2ccc(N(c3ccc(-c4ccccc4)cc3)c3ccc4ccc(-c5ccc6c7c(cccc57)-c5ccccc5-6)cc4c3)cc2)cc1]

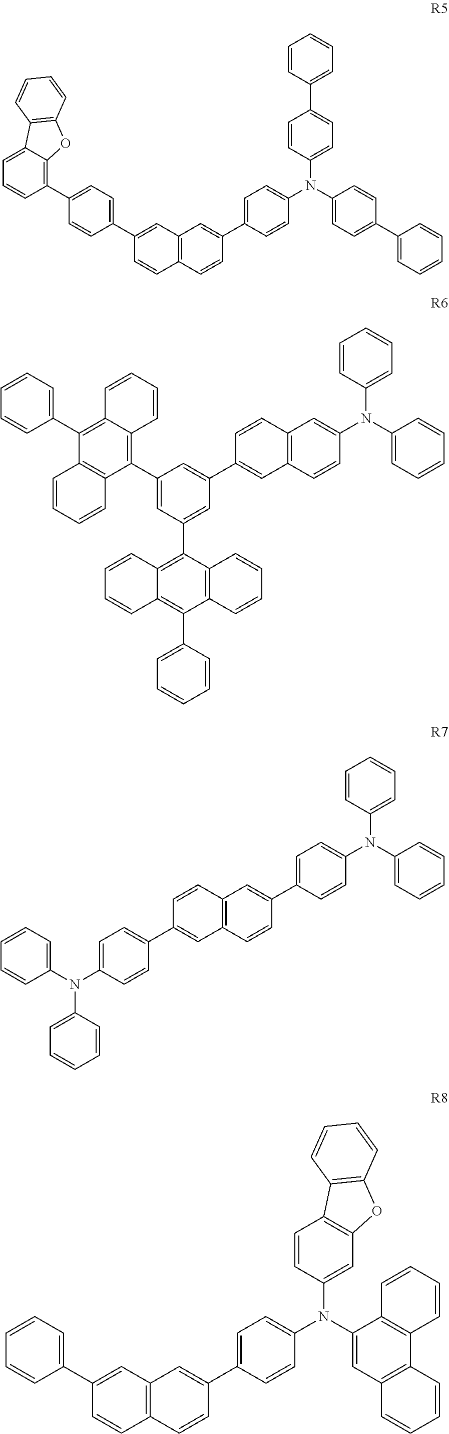 [SMILES: c1ccc(-c2ccc(N(c3ccc(-c4ccccc4)cc3)c3ccc(-c4ccc5ccc(-c6ccc(-c7cccc8c7oc7ccccc78)cc6)cc5c4)cc3)cc2)cc1 ; c1ccc(-c2c3ccccc3c(-c3cc(-c4ccc5cc(N(c6ccccc6)c6ccccc6)ccc5c4)cc(-c4c5ccccc5c(-c5ccccc5)c5ccccc45)c3)c3ccccc23)cc1 ; c1ccc(N(c2ccccc2)c2ccc(-c3ccc4cc(-c5ccc(N(c6ccccc6)c6ccccc6)cc5)ccc4c3)cc2)cc1 ; c1ccc(-c2ccc3ccc(-c4ccc(N(c5ccc6c(c5)oc5ccccc56)c5cc6ccccc6c6ccccc56)cc4)cc3c2)cc1]

The organic electroluminescence devices according to Examples 1 to 15 and Comparative Examples 1 to 8 were manufactured by forming a first electrode using ITO to a thickness of about 150 nm, a hole injection layer using HT1 doped with 2% HIL-M to a thickness of about 10 nm, a hole transport layer using HT1 to a thickness of about 120 nm, an electron blocking layer using the example compounds or the comparative compounds to a thickness of about 10 nm, an emission layer using BH doped with 2% BD to a thickness of about 30 nm, a hole blocking layer using ET1 to a thickness of about 10 nm, an electron transport layer using ET2 to a thickness of about 20 nm, an electron injection layer using LiF to a thickness of about 1 nm, and a second electrode using a Mg/Ag alloy co-deposited at a volumetric ratio of 9:1 to a thickness of about 120 nm. Each layer was formed by a vacuum deposition method.

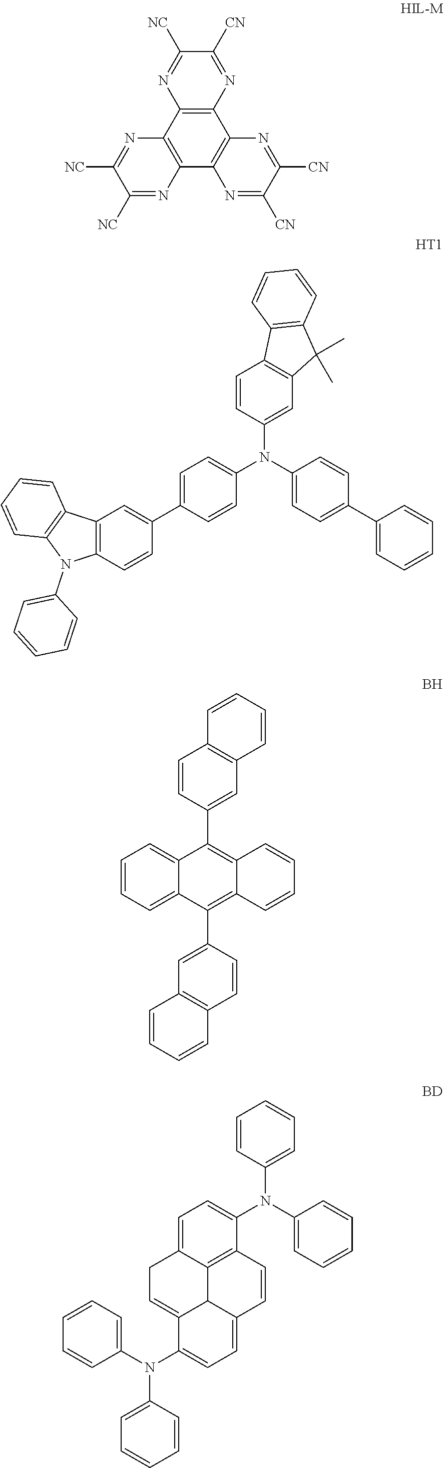

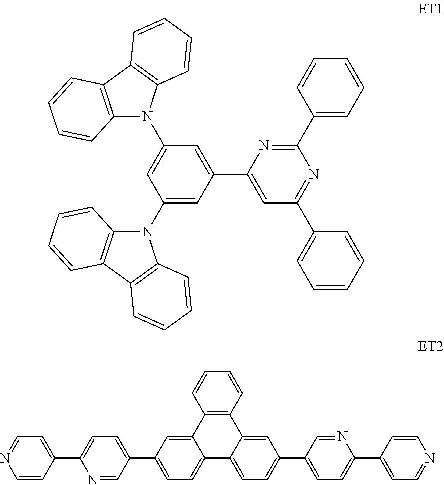 [SMILES: c1ccc(-c2cc(-c3cc(-n4c5ccccc5c5ccccc54)cc(-n4c5ccccc5c5ccccc54)c3)nc(-c3ccccc3)n2)cc1 ; c1ccc2c(c1)c1cc(-c3ccc(-c4ccncc4)nc3)ccc1c1ccc(-c3ccc(-c4ccncc4)nc3)cc21]

The voltage, half-life, emission efficiency, and color coordinate of the organic electroluminescence devices manufactured in Examples 1 to 15 and Comparative Examples 1 to 8 are shown in Table 1 below.

TABLE 1

| | | | Life | Emission | Color |
|---|---|---|---|---|---|
| | Electron | Voltage | LT50 | efficiency | coordinate |
| | blocking layer | (V) | (h) | (cd/A) | CIE (x, y) |
| Example 1 | Example | 4.8 | 5.5 | 189 | 0.141, 0.051 |
| | Compound A4 | | | | |
| Example 2 | Example | 4.6 | 5.5 | 190 | 0.142, 0.051 |
| | Compound A17 | | | | |
| Example 3 | Example | 4.7 | 5.6 | 196 | 0.141, 0.051 |
| | Compound B13 | | | | |
| Example 4 | Example | 4.6 | 5.5 | 195 | 0.141, 0.052 |
| | Compound B20 | | | | |
| Example 5 | Example | 4.6 | 5.4 | 197 | 0.142, 0.052 |
| | Compound B40 | | | | |
| Example 6 | Example | 4.6 | 5.4 | 199 | 0.142, 0.051 |
| | Compound C22 | | | | |
| Example 7 | Example | 4.8 | 5.4 | 196 | 0.141, 0.051 |
| | Compound C51 | | | | |
| Example 8 | Example | 4.8 | 5.5 | 188 | 0.142, 0.052 |
| | Compound D12 | | | | |
| Example 9 | Example | 4.7 | 5.4 | 190 | 0.142, 0.051 |
| | Compound D22 | | | | |
| Example 10 | Example | 4.8 | 5.6 | 184 | 0.141, 0.052 |
| | Compound E3 | | | | |
| Example 11 | Example | 4.8 | 5.6 | 189 | 0.141, 0.052 |
| | Compound E32 | | | | |
| Example 12 | Example | 4.7 | 5.3 | 193 | 0.141, 0.051 |
| | Compound F46 | | | | |
| Example 13 | Example | 4.8 | 5.5 | 192 | 0.141, 0.051 |
| | Compound F53 | | | | |
| Example 14 | Example | 4.8 | 5.6 | 197 | 0.141, 0.052 |
| | Compound G54 | | | | |
| Example 15 | Example | 4.8 | 5.6 | 185 | 0.142, 0.052 |
| | Compound G58 | | | | |
| Comparative | Comparative | 5.0 | 4.9 | 167 | 0.140, 0.052 |
| Example 1 | Compound R1 | | | | |
| Comparative | Comparative | 5.2 | 3.8 | 165 | 0.141, 0.053 |
| Example 2 | Compound R2 | | | | |
| Comparative | Comparative | 5.1 | 4.0 | 167 | 0.142, 0.051 |
| Example 3 | Compound R3 | | | | |
| Comparative | Comparative | 5.3 | 4.2 | 163 | 0.139, 0.049 |
| Example 4 | Compound R4 | | | | |
| Comparative | Comparative | 5.1 | 5.0 | 168 | 0.140, 0.050 |
| Example 5 | Compound R5 | | | | |
| Comparative | Comparative | 5.5 | 3.5 | 155 | 0.137, 0.047 |
| Example 6 | Compound R6 | | | | |
| Comparative | Comparative | 4.9 | 5.0 | 157 | 0.143, 0.053 |
| Example 7 | Compound R7 | | | | |
| Comparative | Comparative | 4.9 | 4.9 | 169 | 0.142, 0.052 |
| Example 8 | Compound R8 | | | | |

In the above table, the emission efficiency was a measured value at a current density of about 10 mA/cm², and the half-life was a value at about 1.0 mA/cm².

Referring to the results in Table 1, it may be found that the organic electroluminescence devices of Examples 1 to 15 had decreased driving voltage, extended life and enhanced efficiency when compared with those of Comparative Examples 1 to 8.

A monoamine compound according to an example embodiment may includes a substituted β-phenylnaphthyl group, which may help provide decreased driving voltage, extended life, and enhanced efficiency of the device. Furthermore, the monoamine compound may achieve an extended device life by the introduction of a naphthyl group having a high thermal resistance and electric charge resistance, with the maintenance of the property of amine group. Furthermore, the monoamine compound may have a bulky naphthyl group substituted with a phenyl group, which may decrease symmetry of molecule and inhibit crystallization, to thereby enhance quality of layers and attain high efficiency of the device.

The organic electroluminescence devices of Examples 1, 2, 8 to 11, 14 and 15 had significantly enhanced efficiency. Without being bound by theory, it is believed that Example Compounds A4, A17, D12, D22, E3, E32, G54 and G58, including a substituent at a position of the naphthyl group, may have a steric electron repulsion between the substituent of a position and the hydrogen atom of other a′ position, and distortion of the naphthyl structure and phenyl group substituted therein may lead to decreased planarity of the whole molecule and inhibit crystallization, thereby improving hole transport property and enhancing the chance of recombining holes and electrons in an emission layer.

Furthermore, the organic electroluminescence devices of Examples 3 to 7, 12 and 13 had significantly extended device life. Without being bound by theory, it is believed that Example Compounds B13, B20, B40, C25, C51, F46 and F53, including a substituent at β position of the naphthyl group, may have a steric conformation close to plane for the naphthyl group and the substituent at β position, which may result in a stabilized radical state due to the delocalized conjugation around amine, thereby enhancing device life.

The organic electroluminescence device of Comparative Example 1 showed decreased device life when compared with those of Examples. Comparative Compound R1 has an amine group substituted at β position of naphthyl group via a linker similar to Example Compounds, but has two phenyl groups substituted in naphthyl group, which may result in largely distributed HOMO in naphthyl group and decreased electron density in amine group, thereby making it difficult to maintain the property of amine to extend device life.

The organic electroluminescence device of Comparative Example 2 uses an amine compound including a naphthyl group but not a phenylnaphthyl group, which results in low electric charge resistance, thereby decreasing device life and emission efficiency due to the insufficient quality of layers.

The organic electroluminescence devices of Comparative Examples 3 and 4 use Comparative Compounds R3 and R4 having an amine group substituted at β position of naphthyl group via a linker similar to Example Compounds, but having polycyclic aromatic groups connected to naphthyl group, contrary to Example Compounds having a phenyl group connected to naphthyl group, which may cause a strong molecular stacking and increased deposition temperature due to the polycyclic aromatic group, thereby resulting in easy thermal decomposition and decreased efficiency and device life, when compared with those of Examples.

The organic electroluminescence device of Comparative Example 6 uses Comparative Compound R6 having an amine group substituted at β position of naphthyl group via a linker similar to Example Compounds, but having a phenyl group with two substituents, which may cause a strong molecular stacking and increased deposition temperature, thereby resulting in easy thermal decomposition and decreased efficiency and device life, when compared with those of Examples.

The organic electroluminescence devices of Comparative Examples 5 and 7 showed especially decreased emission efficiency when compared with those of Examples. Comparative Compound R5 has a naphthyl group substituted with phenyl group that is substituted with dibenzofuran heterocycle, and Comparative Compound R7 is a diamine compound, both of which may disturb carrier balance.

The organic electroluminescence device of Comparative Example 8 showed decreased emission efficiency and device life when compared with those of Examples. Comparative Compound R8 has a nitrogen atom substituted with both of 3-dibenzofuranyl and 9-phenanthryl, which may result in easy thermal decomposition. Comparative Compound R8 has a nitrogen atom substituted with 9-phenanthryl, which may increase molecular stacking, and is further substituted with 3-dibenzofuranyl, which may increase planarity of the whole molecule, thereby causing a strong molecular stacking and increased deposition temperature, which seems to result in easy thermal decomposition and decreased efficiency and device life.

By way of summation and review, in an application of an organic electroluminescence device to a display, decrease of a driving voltage, increase of emission efficiency and extension of life for the organic electroluminescence device are desired, and development of a material which may stably implement these requirements in the organic electroluminescence device is also desired.

Embodiments may provide an organic electroluminescence device and a monoamine compound for an organic electroluminescence device. Embodiments may provide an organic electroluminescence device with high efficiency and a monoamine compound included in a hole transport region of an organic electroluminescence device.

A monoamine compound according to an example embodiment may be used as a material for a hole transport region of an organic electroluminescence device, which may contribute to a decrease of a driving voltage, increase of emission efficiency, and extension of life for the organic electroluminescence device.

The organic electroluminescence device according to an example embodiment may have high efficiency.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. An organic electroluminescence device, comprising:
a first electrode;
a hole transport region on the first electrode;
an emission layer on the hole transport region;
an electron transport region on the emission layer; and
a second electrode on the electron transport region,
wherein the hole transport region includes a monoamine compound represented by the following Formula 1: 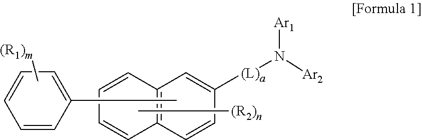
wherein in Formula 1,
Ar₁ and Ar₂ are each independently a substituted or unsubstituted alkyl group having 1 to 10 carbon atoms, a substituted or unsubstituted cycloalkyl group having 3 to 20 ring carbon atoms, a substituted or unsubstituted aryl group having 6 to 30 ring carbon atoms, or a substituted or unsubstituted heteroaryl group having 2 to 30 ring carbon atoms, provided that when any one of Ar₁ and Ar₂ is 3-dibenzofuranyl, the other one of Ar₁ and Ar₂ is not 9-phenanthryl,
L is a substituted or unsubstituted arylene group having 6 to 30 ring carbon atoms, or a substituted or unsubstituted heteroarylene group having 2 to 30 ring carbon atoms,
R₁ is a hydrogen atom, a deuterium atom, a halogen atom, a substituted or unsubstituted alkyl group having 1 to 20 carbon atoms, a substituted or unsubstituted cycloalkyl group having 3 to 20 ring carbon atoms, or a substituted or unsubstituted aryl group having 6 to 30 ring carbon atoms,
R₂ is a hydrogen atom, a deuterium atom, a halogen atom, a substituted or unsubstituted alkyl group having 1 to 20 carbon atoms, or a substituted or unsubstituted cycloalkyl group having 3 to 20 ring carbon atoms,
a is an integer of 0 to 3,
m is an integer of 0 to 1, and
n is an integer of 0 to 6.

2. The organic electroluminescence device as claimed in claim 1, wherein Formula 1 is represented by any one of the following Formulae 2 to 8: 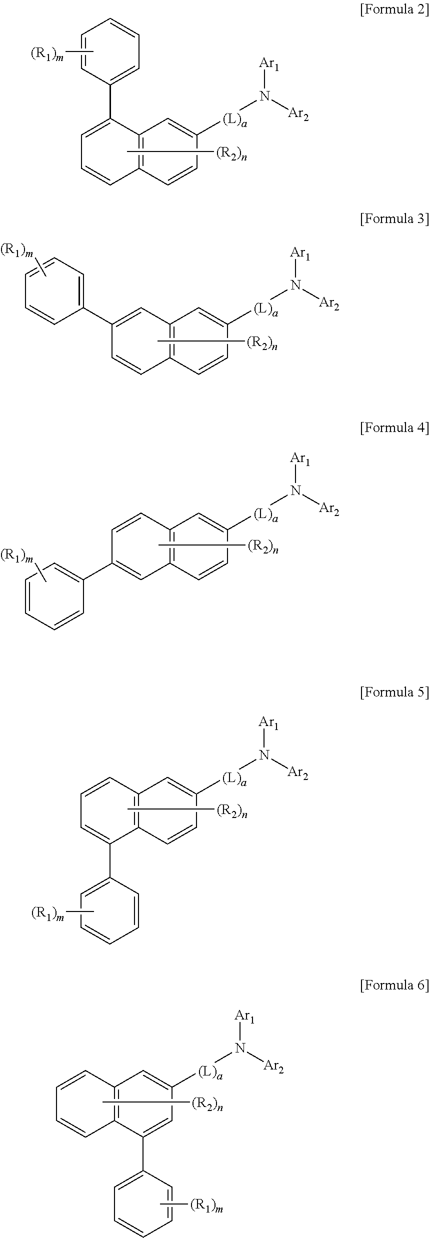 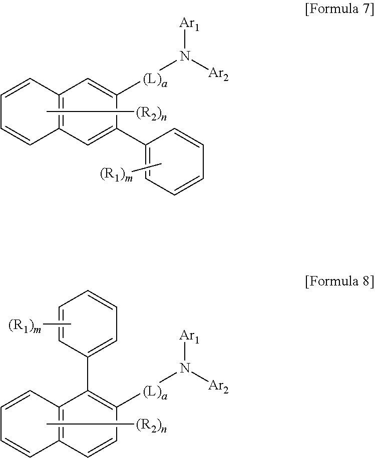
wherein in Formula 2 to 8,
Ar₁, Ar₂, L, R₁, R₂, a, m, and n are the same as defined in Formula 1.

3. The organic electroluminescence device as claimed in claim 1, wherein L is a substituted or unsubstituted arylene group having 6 to 12 ring carbon atoms.

4. The organic electroluminescence device as claimed in claim 3, wherein L is a substituted or unsubstituted phenylene group.

5. The organic electroluminescence device as claimed in claim 1, wherein Ar₁ and Ar₂ are each independently a substituted or unsubstituted aryl group having 6 to 12 ring carbon atoms.

6. The organic electroluminescence device as claimed in claim 5, wherein Ar₁ and Ar₂ are each independently a substituted or unsubstituted phenyl group, a substituted or unsubstituted biphenyl group, a substituted or unsubstituted naphthyl group, or a substituted or unsubstituted fluorenyl group.

7. The organic electroluminescence device as claimed in claim 1, wherein Ar₁ and Ar₂ are each independently a substituted or unsubstituted heteroaryl group having 5 to 12 ring carbon atoms.

8. The organic electroluminescence device as claimed in claim 7, wherein Ar₁ and Ar₂ are each independently a substituted or unsubstituted dibenzofuran group, a substituted or unsubstituted dibenzothiophene group, or a substituted or unsubstituted carbazole group.

9. The organic electroluminescence device as claimed in claim 1, wherein at least one of Ar₁ and Ar₂ are a substituted or unsubstituted 4-dibenzofuran group.

10. The organic electroluminescence device as claimed in claim 1, wherein Formula 1 is represented by the following Formula 9: 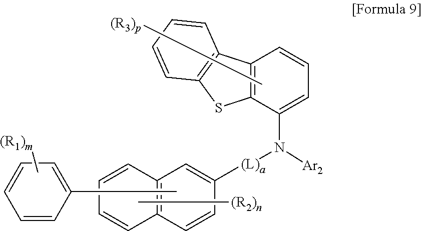
wherein in formula 9, R₃ may be a hydrogen atom, a deuterium atom, a halogen atom, a substituted or unsubstituted alkyl group having 1 to 20 carbon atoms, a substituted or unsubstituted cycloalkyl group having 3 to 20 ring carbon atoms, or a substituted or unsubstituted aryl group having 6 to 30 ring carbon atoms,
p may be an integer of 0 to 7, and
Ar₂, L, R₁, R₂, a, m, and n are the same as defined in Formula 1.

11. The organic electroluminescence device as claimed in claim 1, wherein R₂ is a hydrogen atom or a deuterium atom.

12. The organic electroluminescence device as claimed in claim 1, wherein the hole transport region has a plurality of layers, and a layer of the plurality of layers contacting the emission layer includes the monoamine compound represented by Formula 1.

13. The organic electroluminescence device as claimed in claim 1, wherein the hole transport region includes:
a hole injection layer on the first electrode;
a hole transport layer on the hole injection layer; and
an electron blocking layer on the hole transport layer, the electron blocking layer including the monoamine compound represented by Formula 1.

14. The organic electroluminescence device as claimed in claim 1, wherein the electron transport region includes:
a hole blocking layer on the emission layer;
an electron transport layer on the hole blocking layer; and
an electron injection layer on the electron transport layer.

15. The organic electroluminescence device as claimed in claim 1, wherein the monoamine compound represented by Formula 1 is at least one selected from the group of compounds represented in the following Compound Groups 1 to 7:

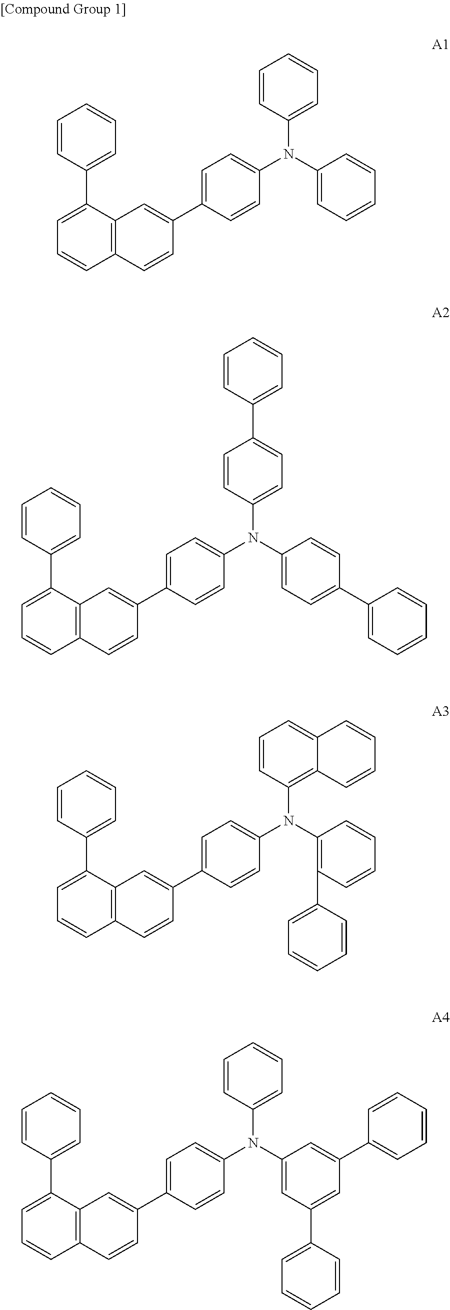 [SMILES: c1ccc(-c2cccc3ccc(-c4ccc(N(c5ccccc5)c5ccccc5)cc4)cc23)cc1 ; c1ccc(-c2ccc(N(c3ccc(-c4ccccc4)cc3)c3ccc(-c4ccc5cccc(-c6ccccc6)c5c4)cc3)cc2)cc1 ; c1ccc(-c2ccccc2N(c2ccc(-c3ccc4cccc(-c5ccccc5)c4c3)cc2)c2cccc3ccccc23)cc1 ; c1ccc(-c2cc(-c3ccccc3)cc(N(c3ccccc3)c3ccc(-c4ccc5cccc(-c6ccccc6)c5c4)cc3)c2)cc1]

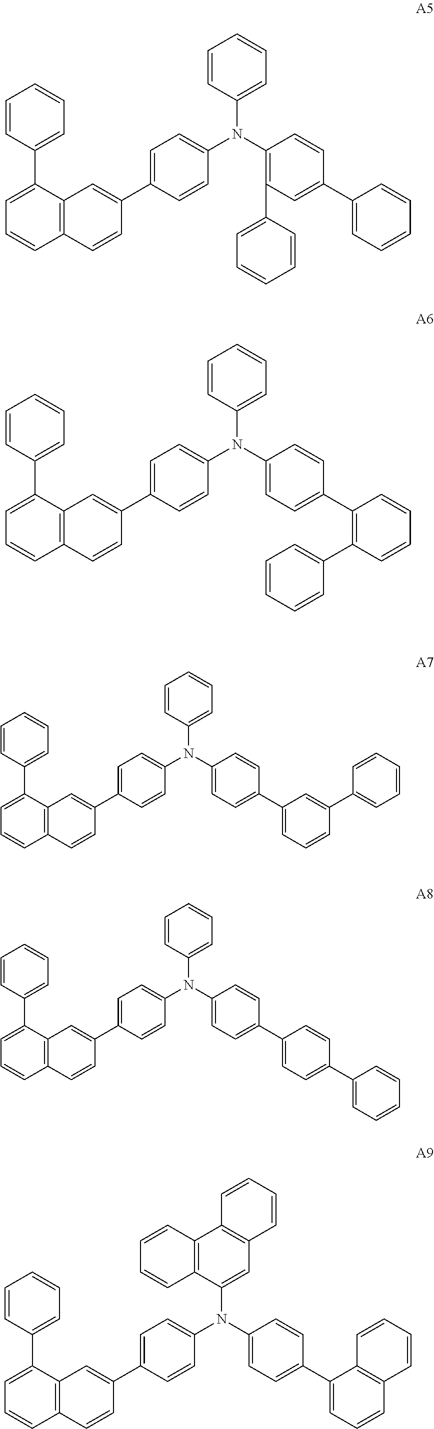 [SMILES: c1ccc(-c2ccc(N(c3ccccc3)c3ccc(-c4ccc5cccc(-c6ccccc6)c5c4)cc3)c(-c3ccccc3)c2)cc1 ; c1ccc(-c2ccccc2-c2ccc(N(c3ccccc3)c3ccc(-c4ccc5cccc(-c6ccccc6)c5c4)cc3)cc2)cc1 ; c1ccc(-c2cccc(-c3ccc(N(c4ccccc4)c4ccc(-c5ccc6cccc(-c7ccccc7)c6c5)cc4)cc3)c2)cc1 ; c1ccc(-c2ccc(-c3ccc(N(c4ccccc4)c4ccc(-c5ccc6cccc(-c7ccccc7)c6c5)cc4)cc3)cc2)cc1 ; c1ccc(-c2cccc3ccc(-c4ccc(N(c5ccc(-c6cccc7ccccc67)cc5)c5cc6ccccc6c6ccccc56)cc4)cc23)cc1]

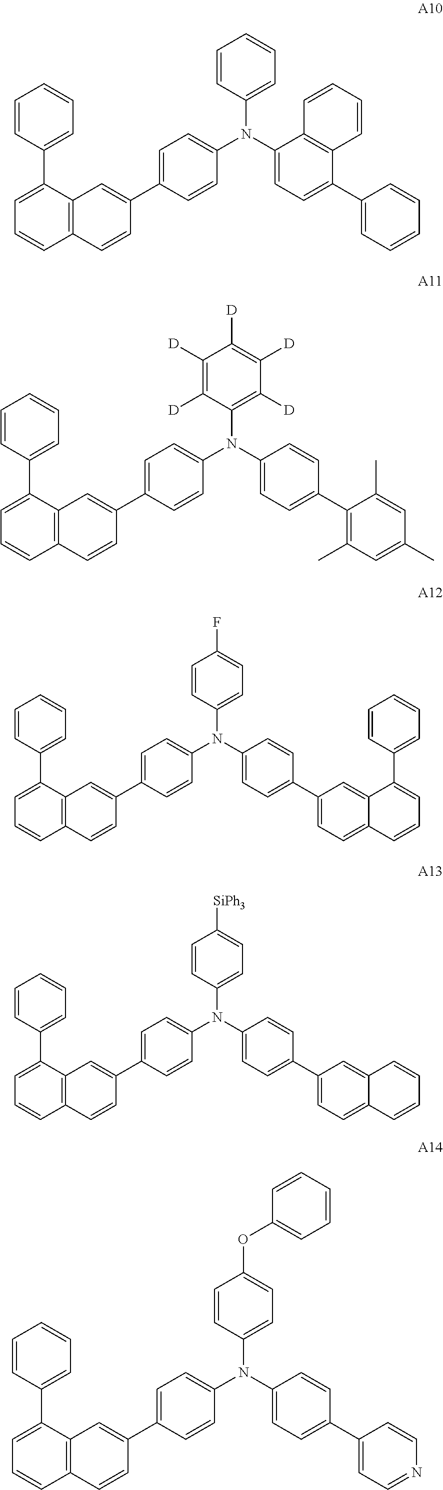

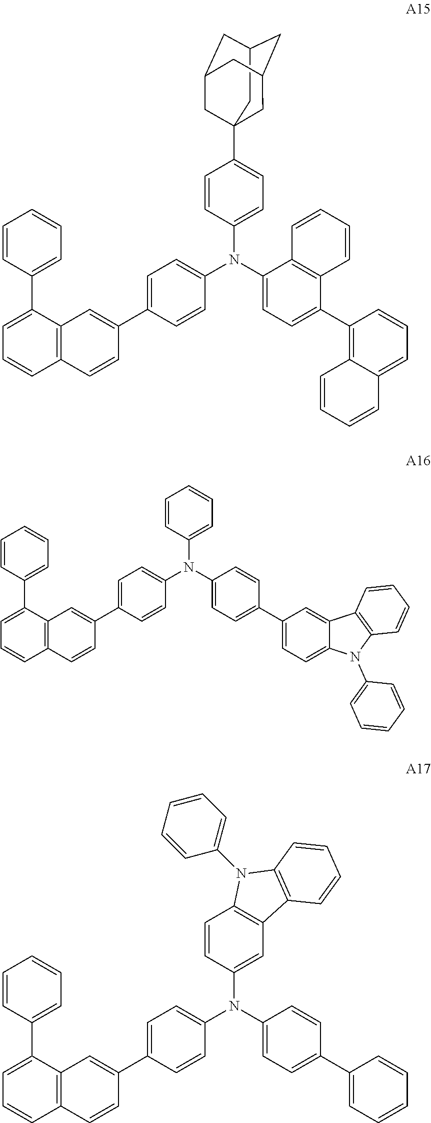 [SMILES: c1ccc(-c2cccc3ccc(-c4ccc(N(c5ccc(C67CC8CC(CC(C8)C6)C7)cc5)c5ccc(-c6cccc7ccccc67)c6ccccc56)cc4)cc23)cc1 ; c1ccc(-c2cccc3ccc(-c4ccc(N(c5ccccc5)c5ccc(-c6ccc7c(c6)c6ccccc6n7-c6ccccc6)cc5)cc4)cc23)cc1 ; c1ccc(-c2ccc(N(c3ccc(-c4ccc5cccc(-c6ccccc6)c5c4)cc3)c3ccc4c(c3)c3ccccc3n4-c3ccccc3)cc2)cc1]

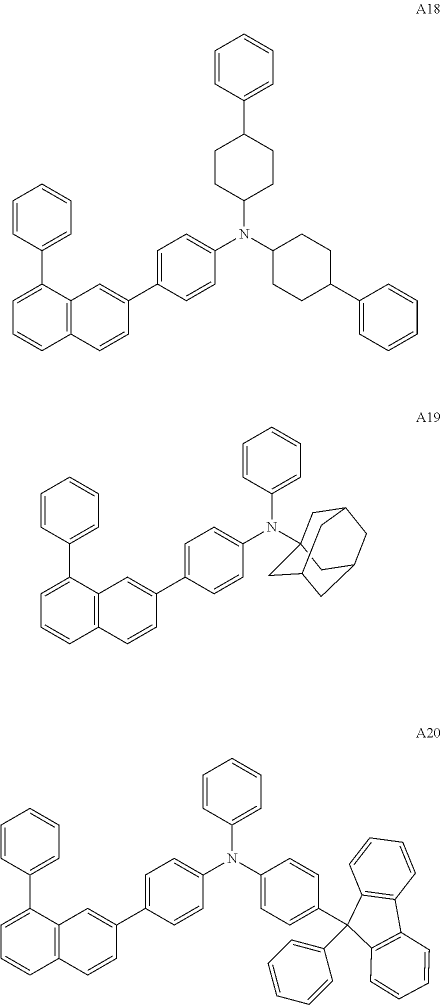 [SMILES: c1ccc(-c2cccc3ccc(-c4ccc(N(C5CCC(c6ccccc6)CC5)C5CCC(c6ccccc6)CC5)cc4)cc23)cc1 ; c1ccc(-c2cccc3ccc(-c4ccc(N(c5ccccc5)C56CC7CC(CC(C7)C5)C6)cc4)cc23)cc1 ; c1ccc(-c2cccc3ccc(-c4ccc(N(c5ccccc5)c5ccc(C6(c7ccccc7)c7ccccc7-c7ccccc76)cc5)cc4)cc23)cc1]

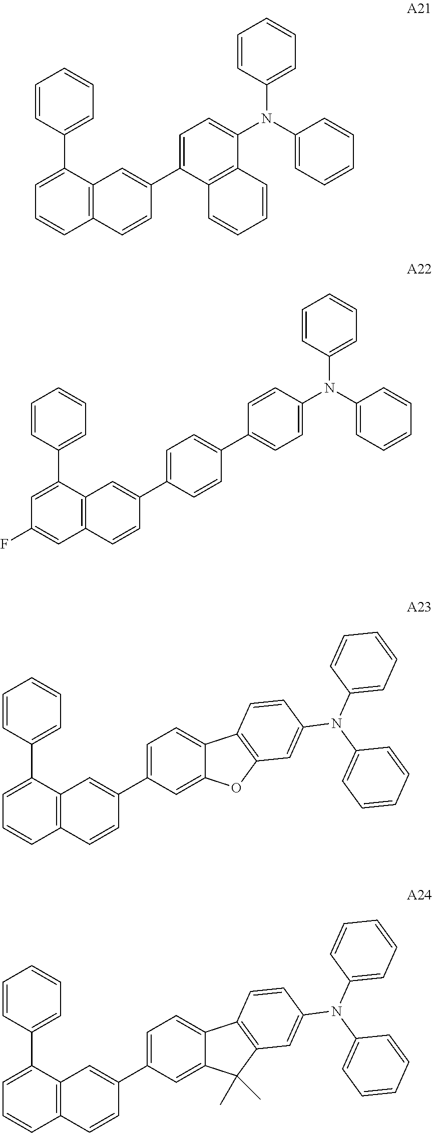 [SMILES: c1ccc(-c2cccc3ccc(-c4ccc(N(c5ccccc5)c5ccccc5)c5ccccc45)cc23)cc1 ; Fc1cc(-c2ccccc2)c2cc(-c3ccc(-c4ccc(N(c5ccccc5)c5ccccc5)cc4)cc3)ccc2c1 ; c1ccc(-c2cccc3ccc(-c4ccc5c(c4)oc4cc(N(c6ccccc6)c6ccccc6)ccc45)cc23)cc1 ; CC1(C)c2cc(-c3ccc4cccc(-c5ccccc5)c4c3)ccc2-c2ccc(N(c3ccccc3)c3ccccc3)cc21]

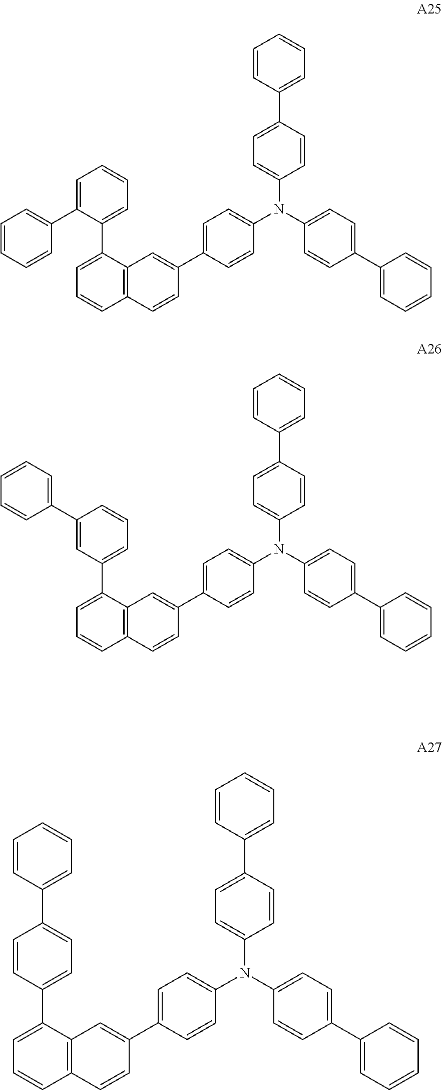 [SMILES: c1ccc(-c2ccc(N(c3ccc(-c4ccccc4)cc3)c3ccc(-c4ccc5cccc(-c6ccccc6-c6ccccc6)c5c4)cc3)cc2)cc1 ; c1ccc(-c2ccc(N(c3ccc(-c4ccccc4)cc3)c3ccc(-c4ccc5cccc(-c6cccc(-c7ccccc7)c6)c5c4)cc3)cc2)cc1 ; c1ccc(-c2ccc(-c3cccc4ccc(-c5ccc(N(c6ccc(-c7ccccc7)cc6)c6ccc(-c7ccccc7)cc6)cc5)cc34)cc2)cc1]

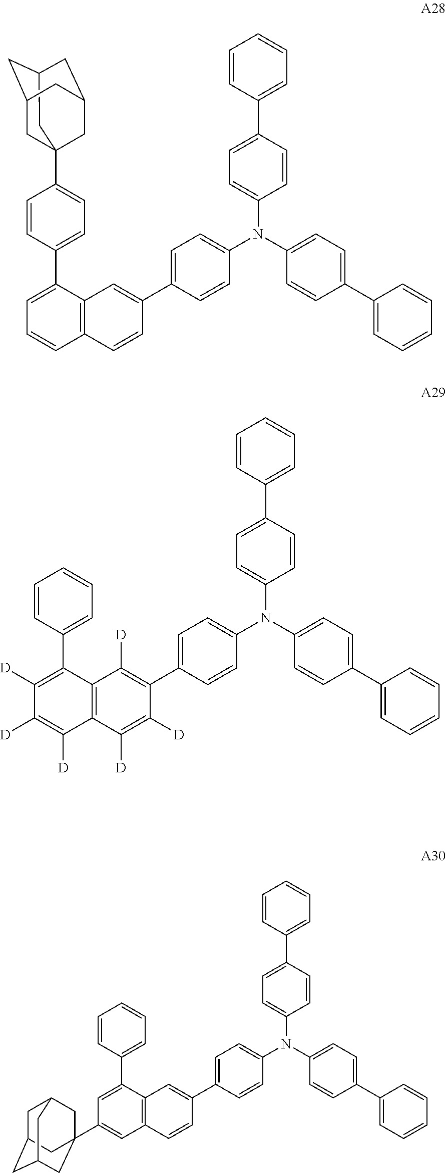 [SMILES: c1ccc(-c2ccc(N(c3ccc(-c4ccccc4)cc3)c3ccc(-c4ccc5cccc(-c6ccc(C78CC9CC(CC(C9)C7)C8)cc6)c5c4)cc3)cc2)cc1 ; [2H]c1c([2H])c(-c2ccccc2)c2c([2H])c(-c3ccc(N(c4ccc(-c5ccccc5)cc4)c4ccc(-c5ccccc5)cc4)cc3)c([2H])c([2H])c2c1[2H] ; c1ccc(-c2ccc(N(c3ccc(-c4ccccc4)cc3)c3ccc(-c4ccc5cc(C67CC8CC(CC(C8)C6)C7)cc(-c6ccccc6)c5c4)cc3)cc2)cc1]

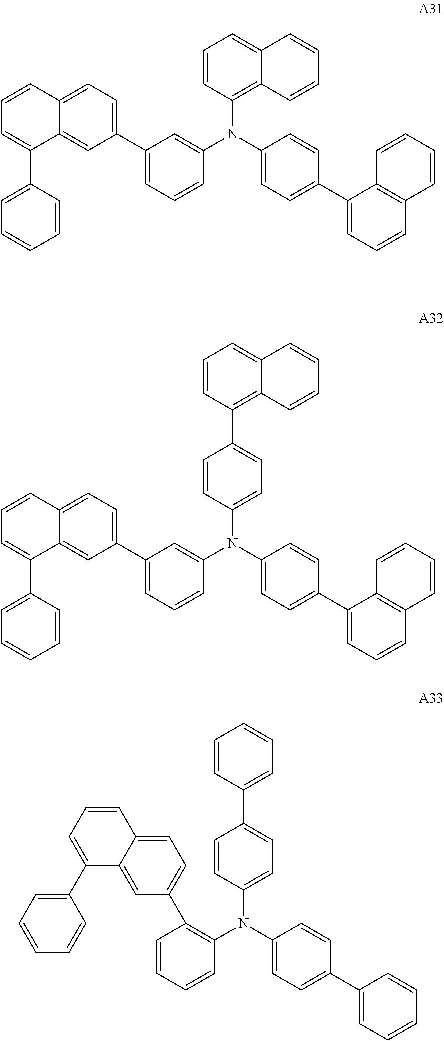 [SMILES: c1ccc(-c2cccc3ccc(-c4cccc(N(c5ccc(-c6cccc7ccccc67)cc5)c5cccc6ccccc56)c4)cc23)cc1 ; c1ccc(-c2cccc3ccc(-c4cccc(N(c5ccc(-c6cccc7ccccc67)cc5)c5ccc(-c6cccc7ccccc67)cc5)c4)cc23)cc1 ; c1ccc(-c2ccc(N(c3ccc(-c4ccccc4)cc3)c3ccccc3-c3ccc4cccc(-c5ccccc5)c4c3)cc2)cc1]

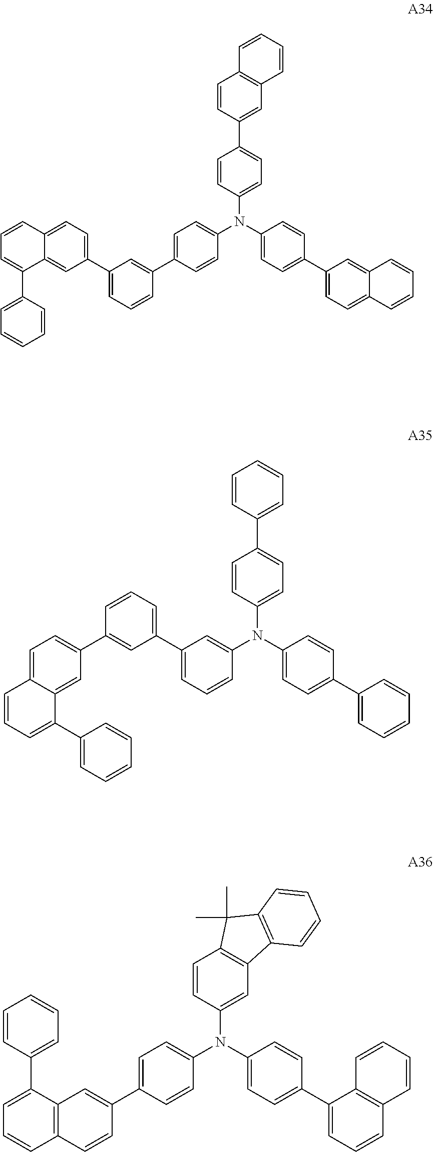 [SMILES: c1ccc(-c2cccc3ccc(-c4cccc(-c5ccc(N(c6ccc(-c7ccc8ccccc8c7)cc6)c6ccc(-c7ccc8ccccc8c7)cc6)cc5)c4)cc23)cc1 ; c1ccc(-c2ccc(N(c3ccc(-c4ccccc4)cc3)c3cccc(-c4cccc(-c5ccc6cccc(-c7ccccc7)c6c5)c4)c3)cc2)cc1 ; CC1(C)c2ccccc2-c2cc(N(c3ccc(-c4ccc5cccc(-c6ccccc6)c5c4)cc3)c3ccc(-c4cccc5ccccc45)cc3)ccc21]

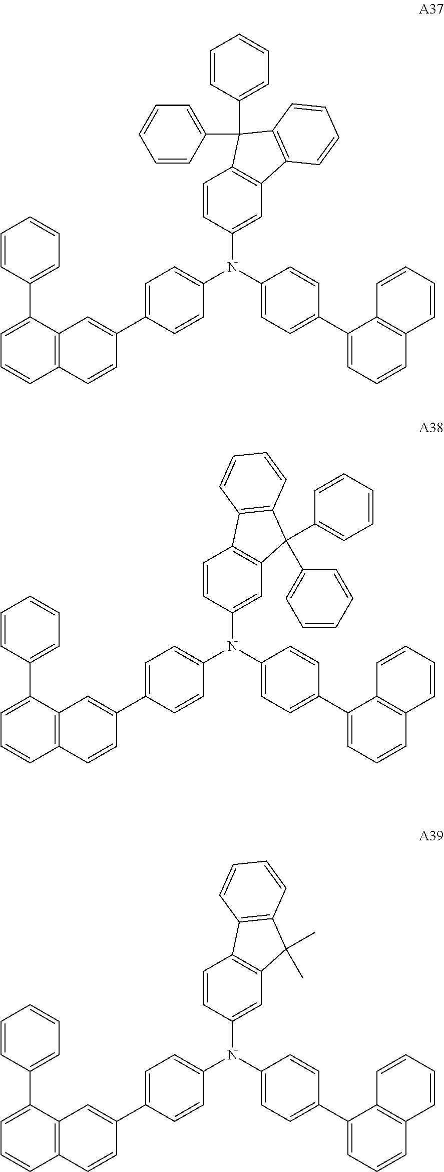 [SMILES: c1ccc(-c2cccc3ccc(-c4ccc(N(c5ccc(-c6cccc7ccccc67)cc5)c5ccc6c(c5)-c5ccccc5C6(c5ccccc5)c5ccccc5)cc4)cc23)cc1 ; c1ccc(-c2cccc3ccc(-c4ccc(N(c5ccc(-c6cccc7ccccc67)cc5)c5ccc6c(c5)C(c5ccccc5)(c5ccccc5)c5ccccc5-6)cc4)cc23)cc1 ; CC1(C)c2ccccc2-c2ccc(N(c3ccc(-c4ccc5cccc(-c6ccccc6)c5c4)cc3)c3ccc(-c4cccc5ccccc45)cc3)cc21]

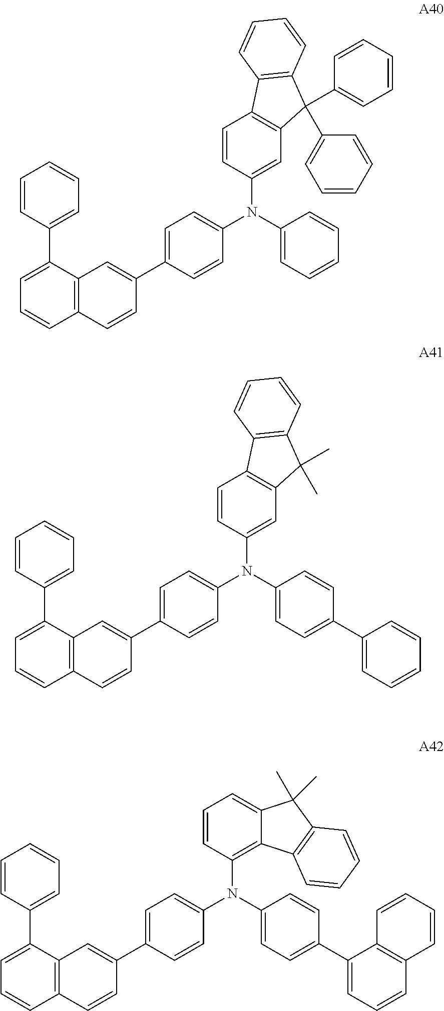 [SMILES: c1ccc(-c2cccc3ccc(-c4ccc(N(c5ccccc5)c5ccc6c(c5)C(c5ccccc5)(c5ccccc5)c5ccccc5-6)cc4)cc23)cc1 ; CC1(C)c2ccccc2-c2ccc(N(c3ccc(-c4ccccc4)cc3)c3ccc(-c4ccc5cccc(-c6ccccc6)c5c4)cc3)cc21 ; CC1(C)c2ccccc2-c2c(N(c3ccc(-c4ccc5cccc(-c6ccccc6)c5c4)cc3)c3ccc(-c4cccc5ccccc45)cc3)cccc21]

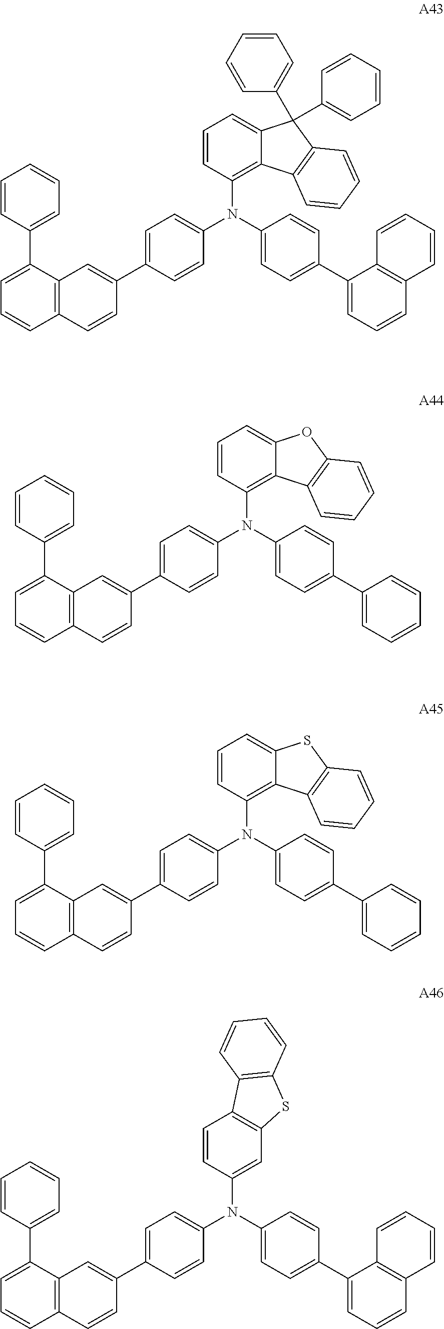 [SMILES: c1ccc(-c2cccc3ccc(-c4ccc(N(c5ccc(-c6cccc7ccccc67)cc5)c5cccc6c5-c5ccccc5C6(c5ccccc5)c5ccccc5)cc4)cc23)cc1 ; c1ccc(-c2ccc(N(c3ccc(-c4ccc5cccc(-c6ccccc6)c5c4)cc3)c3cccc4oc5ccccc5c34)cc2)cc1 ; c1ccc(-c2ccc(N(c3ccc(-c4ccc5cccc(-c6ccccc6)c5c4)cc3)c3cccc4sc5ccccc5c34)cc2)cc1 ; c1ccc(-c2cccc3ccc(-c4ccc(N(c5ccc(-c6cccc7ccccc67)cc5)c5ccc6c(c5)sc5ccccc56)cc4)cc23)cc1]

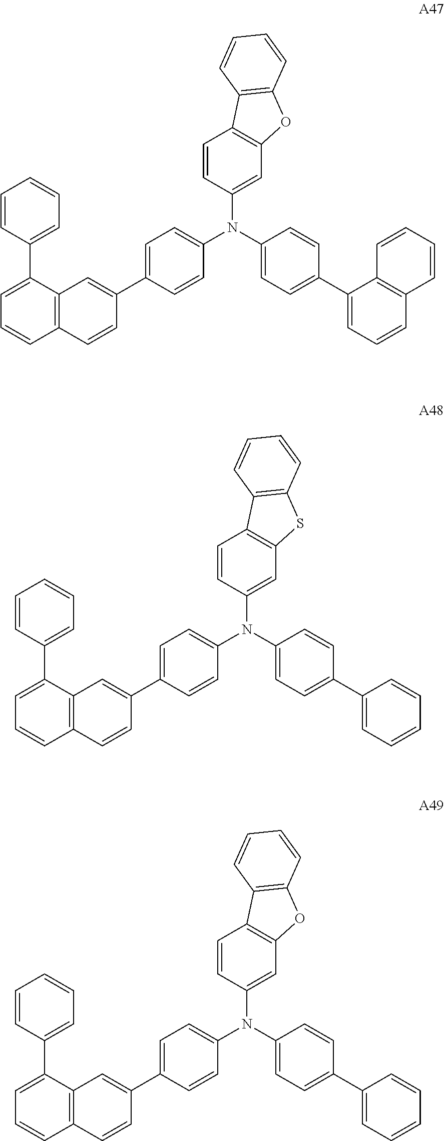 [SMILES: c1ccc(-c2cccc3ccc(-c4ccc(N(c5ccc(-c6cccc7ccccc67)cc5)c5ccc6c(c5)oc5ccccc56)cc4)cc23)cc1 ; c1ccc(-c2ccc(N(c3ccc(-c4ccc5cccc(-c6ccccc6)c5c4)cc3)c3ccc4c(c3)sc3ccccc34)cc2)cc1 ; c1ccc(-c2ccc(N(c3ccc(-c4ccc5cccc(-c6ccccc6)c5c4)cc3)c3ccc4c(c3)oc3ccccc34)cc2)cc1]

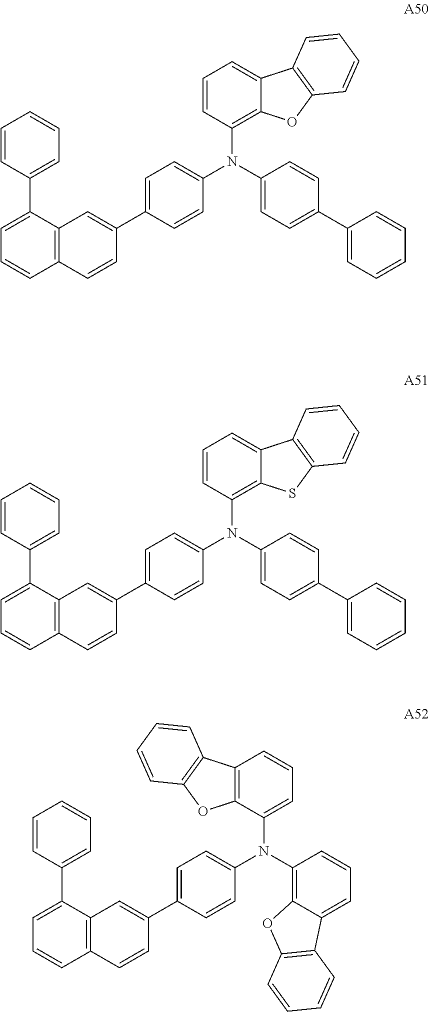 [SMILES: c1ccc(-c2ccc(N(c3ccc(-c4ccc5cccc(-c6ccccc6)c5c4)cc3)c3cccc4c3oc3ccccc34)cc2)cc1 ; c1ccc(-c2ccc(N(c3ccc(-c4ccc5cccc(-c6ccccc6)c5c4)cc3)c3cccc4c3sc3ccccc34)cc2)cc1 ; c1ccc(-c2cccc3ccc(-c4ccc(N(c5cccc6c5oc5ccccc56)c5cccc6c5oc5ccccc56)cc4)cc23)cc1]

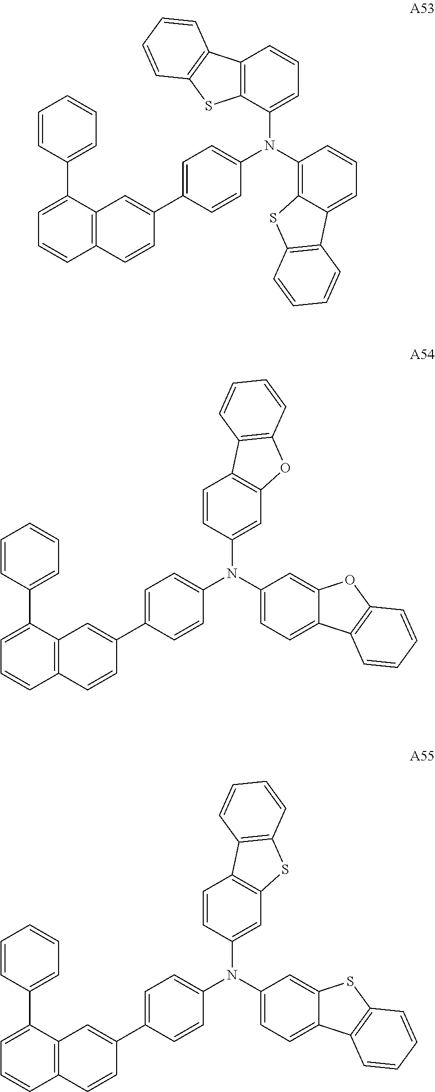 [SMILES: c1ccc(-c2cccc3ccc(-c4ccc(N(c5cccc6c5sc5ccccc56)c5cccc6c5sc5ccccc56)cc4)cc23)cc1 ; c1ccc(-c2cccc3ccc(-c4ccc(N(c5ccc6c(c5)oc5ccccc56)c5ccc6c(c5)oc5ccccc56)cc4)cc23)cc1 ; c1ccc(-c2cccc3ccc(-c4ccc(N(c5ccc6c(c5)sc5ccccc56)c5ccc6c(c5)sc5ccccc56)cc4)cc23)cc1]

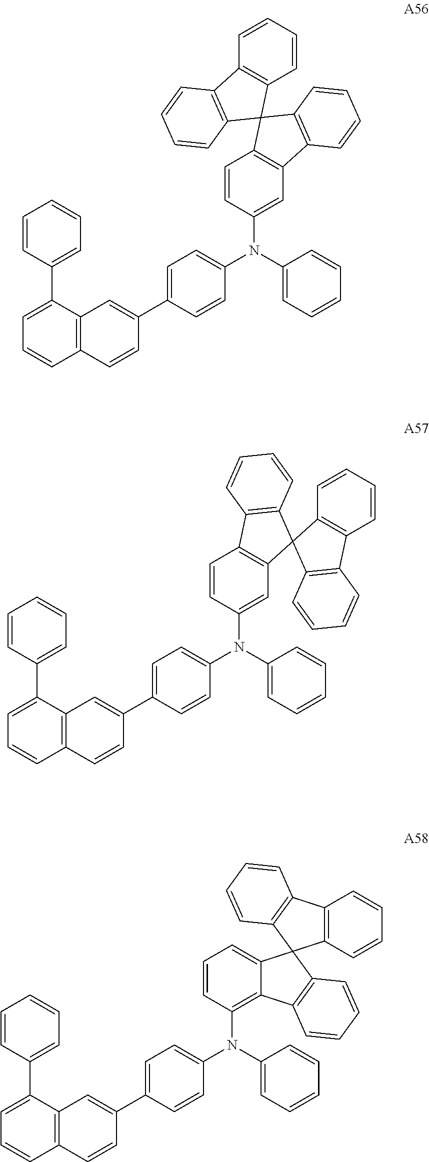 [SMILES: c1ccc(-c2cccc3ccc(-c4ccc(N(c5ccccc5)c5ccc6c(c5)-c5ccccc5C65c6ccccc6-c6ccccc65)cc4)cc23)cc1 ; c1ccc(-c2cccc3ccc(-c4ccc(N(c5ccccc5)c5ccc6c(c5)C5(c7ccccc7-c7ccccc75)c5ccccc5-6)cc4)cc23)cc1 ; c1ccc(-c2cccc3ccc(-c4ccc(N(c5ccccc5)c5cccc6c5-c5ccccc5C65c6ccccc6-c6ccccc65)cc4)cc23)cc1]

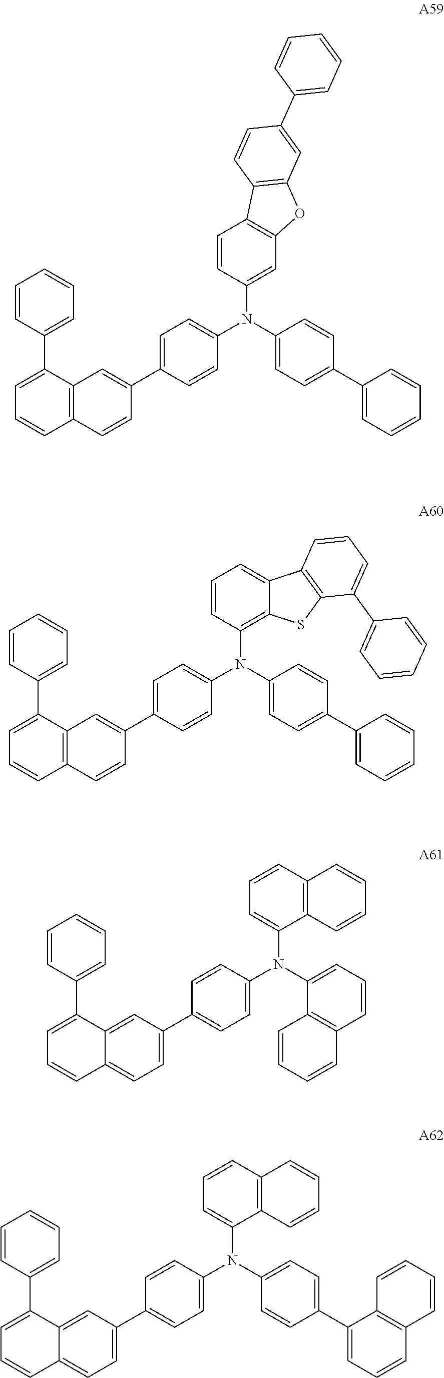 [SMILES: c1ccc(-c2ccc(N(c3ccc(-c4ccc5cccc(-c6ccccc6)c5c4)cc3)c3ccc4c(c3)oc3cc(-c5ccccc5)ccc34)cc2)cc1 ; c1ccc(-c2ccc(N(c3ccc(-c4ccc5cccc(-c6ccccc6)c5c4)cc3)c3cccc4c3sc3c(-c5ccccc5)cccc34)cc2)cc1 ; c1ccc(-c2cccc3ccc(-c4ccc(N(c5cccc6ccccc56)c5cccc6ccccc56)cc4)cc23)cc1 ; c1ccc(-c2cccc3ccc(-c4ccc(N(c5ccc(-c6cccc7ccccc67)cc5)c5cccc6ccccc56)cc4)cc23)cc1]

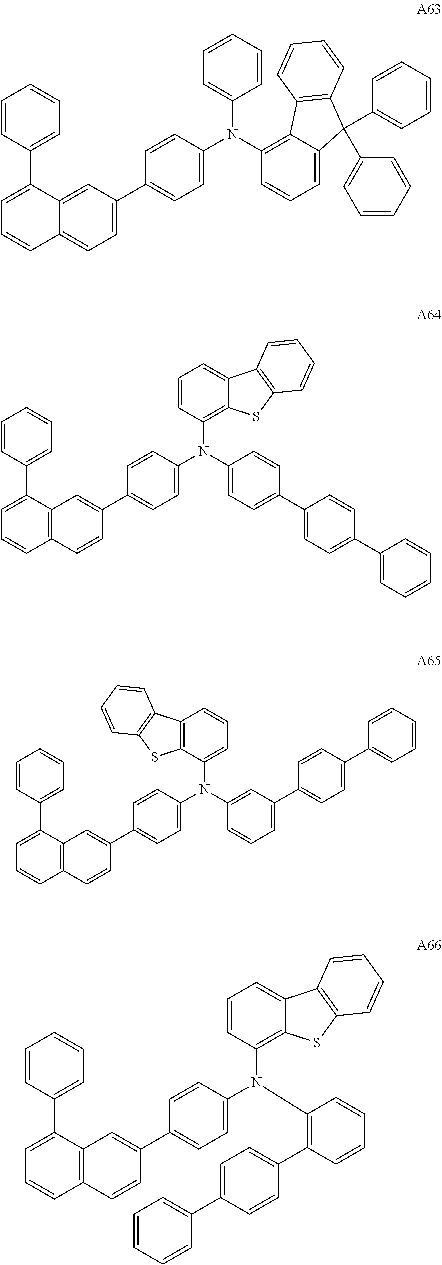 [SMILES: c1ccc(-c2cccc3ccc(-c4ccc(N(c5ccccc5)c5cccc6c5-c5ccccc5C6(c5ccccc5)c5ccccc5)cc4)cc23)cc1 ; c1ccc(-c2ccc(-c3ccc(N(c4ccc(-c5ccc6cccc(-c7ccccc7)c6c5)cc4)c4cccc5c4sc4ccccc45)cc3)cc2)cc1 ; c1ccc(-c2ccc(-c3cccc(N(c4ccc(-c5ccc6cccc(-c7ccccc7)c6c5)cc4)c4cccc5c4sc4ccccc45)c3)cc2)cc1 ; c1ccc(-c2ccc(-c3ccccc3N(c3ccc(-c4ccc5cccc(-c6ccccc6)c5c4)cc3)c3cccc4c3sc3ccccc34)cc2)cc1]

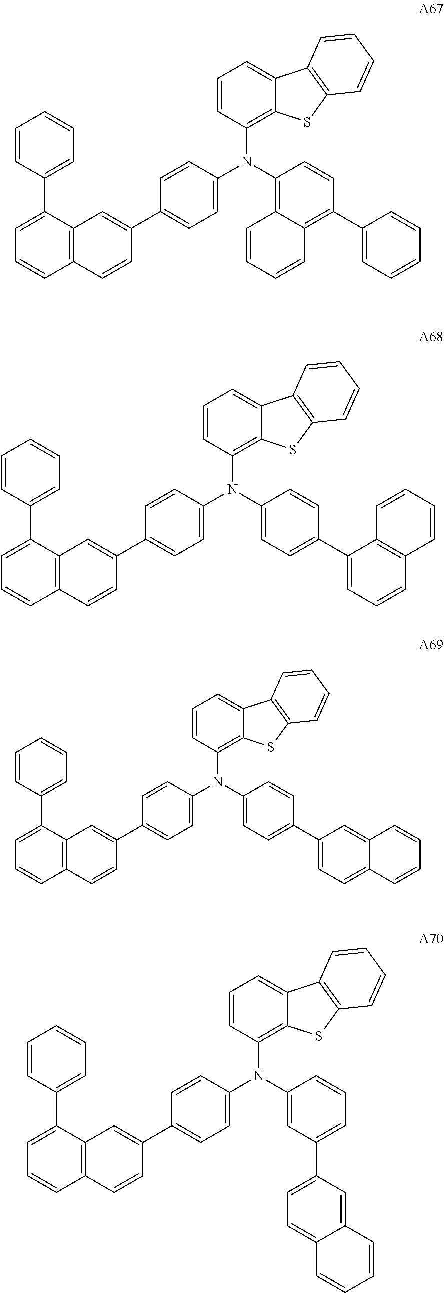 [SMILES: c1ccc(-c2cccc3ccc(-c4ccc(N(c5ccc(-c6ccccc6)c6ccccc56)c5cccc6c5sc5ccccc56)cc4)cc23)cc1 ; c1ccc(-c2cccc3ccc(-c4ccc(N(c5ccc(-c6cccc7ccccc67)cc5)c5cccc6c5sc5ccccc56)cc4)cc23)cc1 ; c1ccc(-c2cccc3ccc(-c4ccc(N(c5ccc(-c6ccc7ccccc7c6)cc5)c5cccc6c5sc5ccccc56)cc4)cc23)cc1 ; c1ccc(-c2cccc3ccc(-c4ccc(N(c5cccc(-c6ccc7ccccc7c6)c5)c5cccc6c5sc5ccccc56)cc4)cc23)cc1]

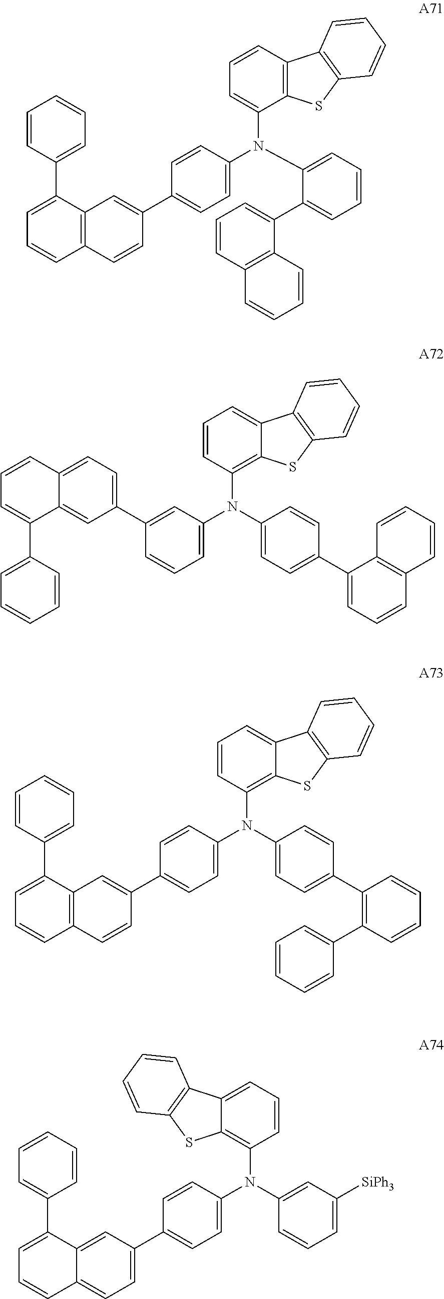

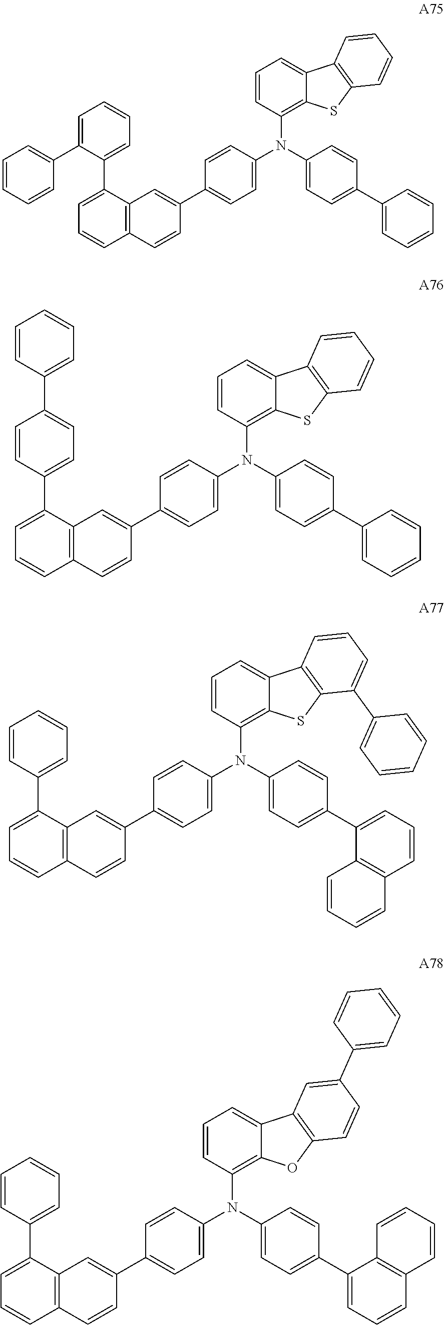 [SMILES: c1ccc(-c2ccc(N(c3ccc(-c4ccc5cccc(-c6ccccc6-c6ccccc6)c5c4)cc3)c3cccc4c3sc3ccccc34)cc2)cc1 ; c1ccc(-c2ccc(-c3cccc4ccc(-c5ccc(N(c6ccc(-c7ccccc7)cc6)c6cccc7c6sc6ccccc67)cc5)cc34)cc2)cc1 ; c1ccc(-c2cccc3ccc(-c4ccc(N(c5ccc(-c6cccc7ccccc67)cc5)c5cccc6c5sc5c(-c7ccccc7)cccc56)cc4)cc23)cc1 ; c1ccc(-c2ccc3oc4c(N(c5ccc(-c6ccc7cccc(-c8ccccc8)c7c6)cc5)c5ccc(-c6cccc7ccccc67)cc5)cccc4c3c2)cc1]

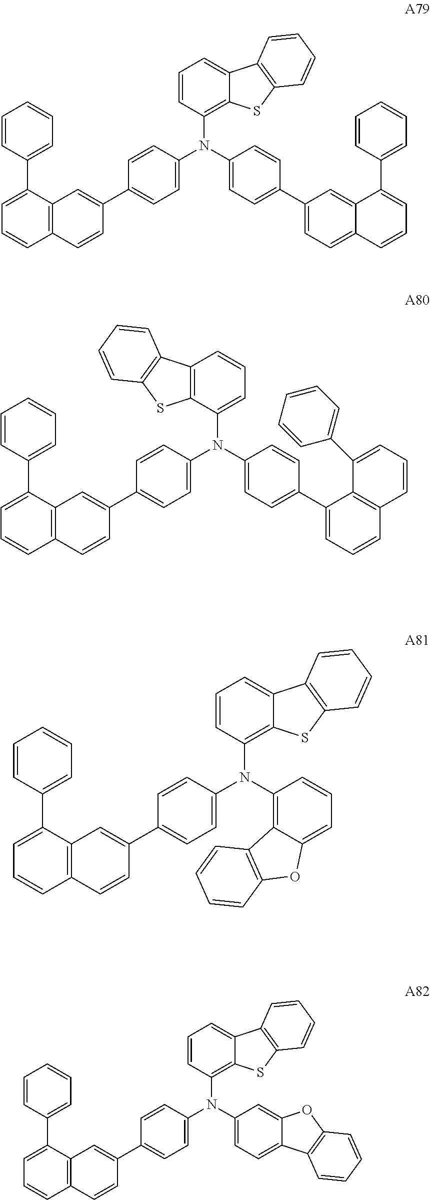 [SMILES: c1ccc(-c2cccc3ccc(-c4ccc(N(c5ccc(-c6ccc7cccc(-c8ccccc8)c7c6)cc5)c5cccc6c5sc5ccccc56)cc4)cc23)cc1 ; c1ccc(-c2cccc3ccc(-c4ccc(N(c5ccc(-c6cccc7cccc(-c8ccccc8)c67)cc5)c5cccc6c5sc5ccccc56)cc4)cc23)cc1 ; c1ccc(-c2cccc3ccc(-c4ccc(N(c5cccc6c5sc5ccccc56)c5cccc6oc7ccccc7c56)cc4)cc23)cc1 ; c1ccc(-c2cccc3ccc(-c4ccc(N(c5ccc6c(c5)oc5ccccc56)c5cccc6c5sc5ccccc56)cc4)cc23)cc1]

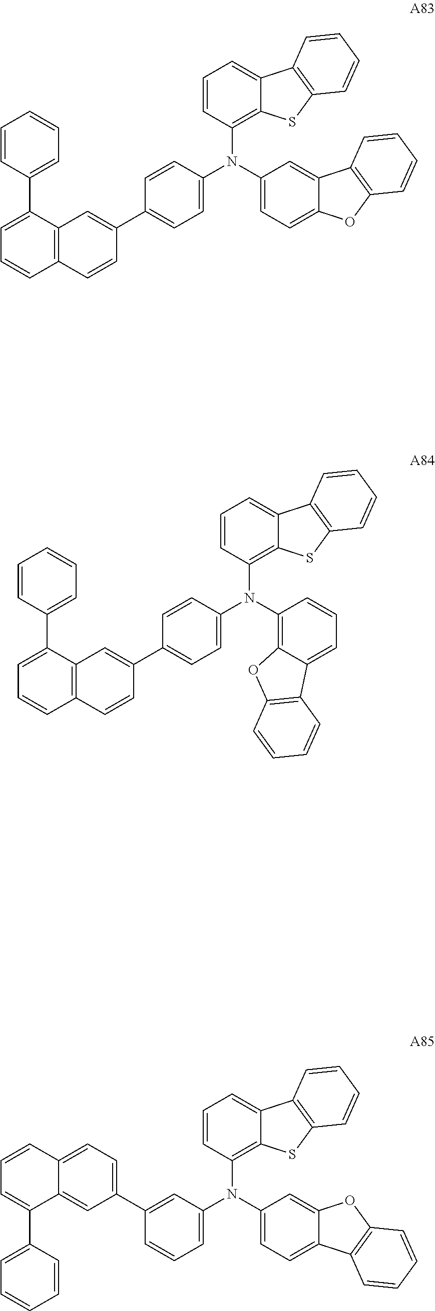 [SMILES: c1ccc(-c2cccc3ccc(-c4ccc(N(c5ccc6oc7ccccc7c6c5)c5cccc6c5sc5ccccc56)cc4)cc23)cc1 ; c1ccc(-c2cccc3ccc(-c4ccc(N(c5cccc6c5oc5ccccc56)c5cccc6c5sc5ccccc56)cc4)cc23)cc1 ; c1ccc(-c2cccc3ccc(-c4cccc(N(c5ccc6c(c5)oc5ccccc56)c5cccc6c5sc5ccccc56)c4)cc23)cc1]

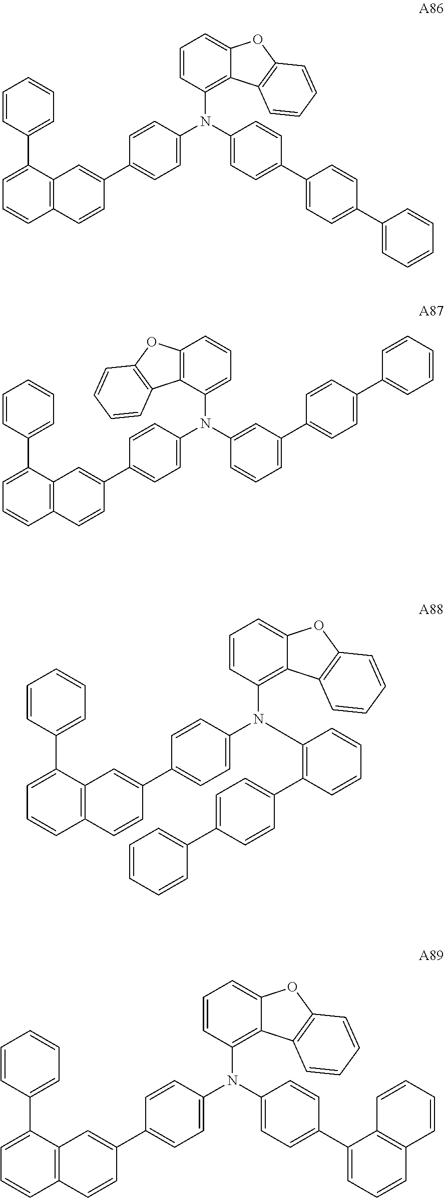 [SMILES: c1ccc(-c2ccc(-c3ccc(N(c4ccc(-c5ccc6cccc(-c7ccccc7)c6c5)cc4)c4cccc5oc6ccccc6c45)cc3)cc2)cc1 ; c1ccc(-c2ccc(-c3cccc(N(c4ccc(-c5ccc6cccc(-c7ccccc7)c6c5)cc4)c4cccc5oc6ccccc6c45)c3)cc2)cc1 ; c1ccc(-c2ccc(-c3ccccc3N(c3ccc(-c4ccc5cccc(-c6ccccc6)c5c4)cc3)c3cccc4oc5ccccc5c34)cc2)cc1 ; c1ccc(-c2cccc3ccc(-c4ccc(N(c5ccc(-c6cccc7ccccc67)cc5)c5cccc6oc7ccccc7c56)cc4)cc23)cc1]

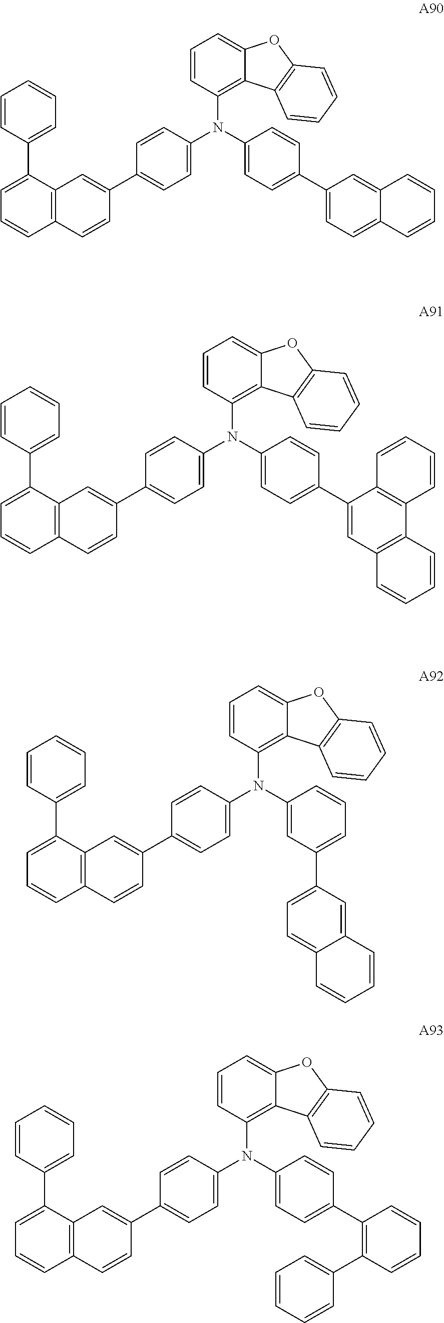 [SMILES: c1ccc(-c2cccc3ccc(-c4ccc(N(c5ccc(-c6ccc7ccccc7c6)cc5)c5cccc6oc7ccccc7c56)cc4)cc23)cc1 ; c1ccc(-c2cccc3ccc(-c4ccc(N(c5ccc(-c6cc7ccccc7c7ccccc67)cc5)c5cccc6oc7ccccc7c56)cc4)cc23)cc1 ; c1ccc(-c2cccc3ccc(-c4ccc(N(c5cccc(-c6ccc7ccccc7c6)c5)c5cccc6oc7ccccc7c56)cc4)cc23)cc1 ; c1ccc(-c2ccccc2-c2ccc(N(c3ccc(-c4ccc5cccc(-c6ccccc6)c5c4)cc3)c3cccc4oc5ccccc5c34)cc2)cc1]

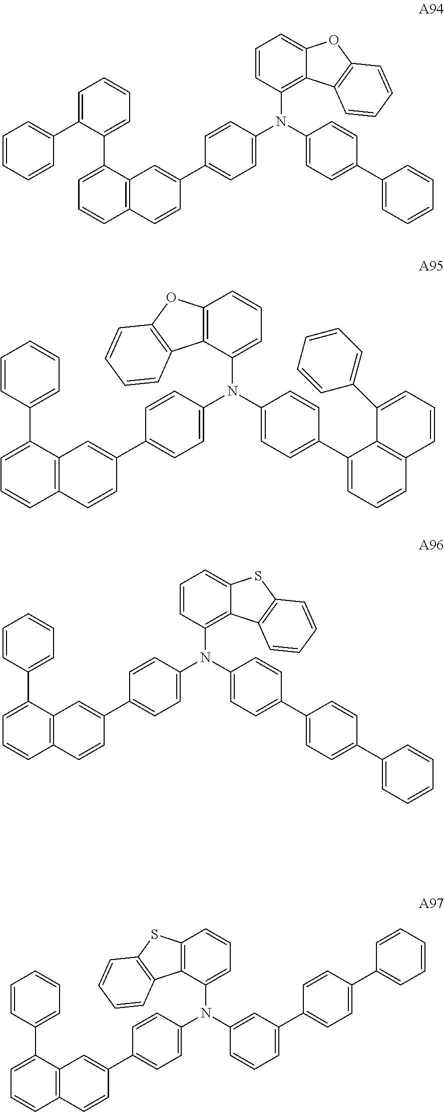 [SMILES: c1ccc(-c2ccc(N(c3ccc(-c4ccc5cccc(-c6ccccc6-c6ccccc6)c5c4)cc3)c3cccc4oc5ccccc5c34)cc2)cc1 ; c1ccc(-c2cccc3ccc(-c4ccc(N(c5ccc(-c6cccc7cccc(-c8ccccc8)c67)cc5)c5cccc6oc7ccccc7c56)cc4)cc23)cc1 ; c1ccc(-c2ccc(-c3ccc(N(c4ccc(-c5ccc6cccc(-c7ccccc7)c6c5)cc4)c4cccc5sc6ccccc6c45)cc3)cc2)cc1 ; c1ccc(-c2ccc(-c3cccc(N(c4ccc(-c5ccc6cccc(-c7ccccc7)c6c5)cc4)c4cccc5sc6ccccc6c45)c3)cc2)cc1]

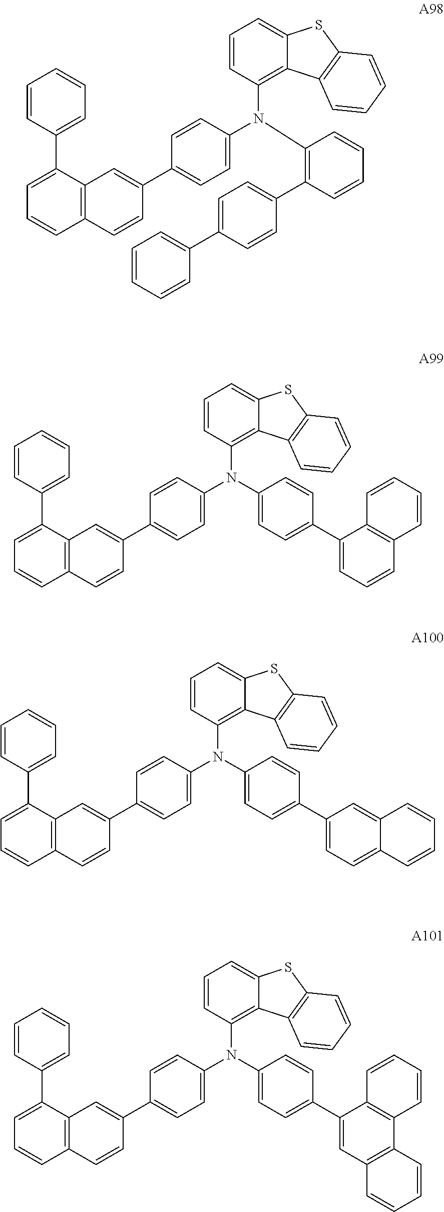 [SMILES: c1ccc(-c2ccc(-c3ccccc3N(c3ccc(-c4ccc5cccc(-c6ccccc6)c5c4)cc3)c3cccc4sc5ccccc5c34)cc2)cc1 ; c1ccc(-c2cccc3ccc(-c4ccc(N(c5ccc(-c6cccc7ccccc67)cc5)c5cccc6sc7ccccc7c56)cc4)cc23)cc1 ; c1ccc(-c2cccc3ccc(-c4ccc(N(c5ccc(-c6ccc7ccccc7c6)cc5)c5cccc6sc7ccccc7c56)cc4)cc23)cc1 ; c1ccc(-c2cccc3ccc(-c4ccc(N(c5ccc(-c6cc7ccccc7c7ccccc67)cc5)c5cccc6sc7ccccc7c56)cc4)cc23)cc1]

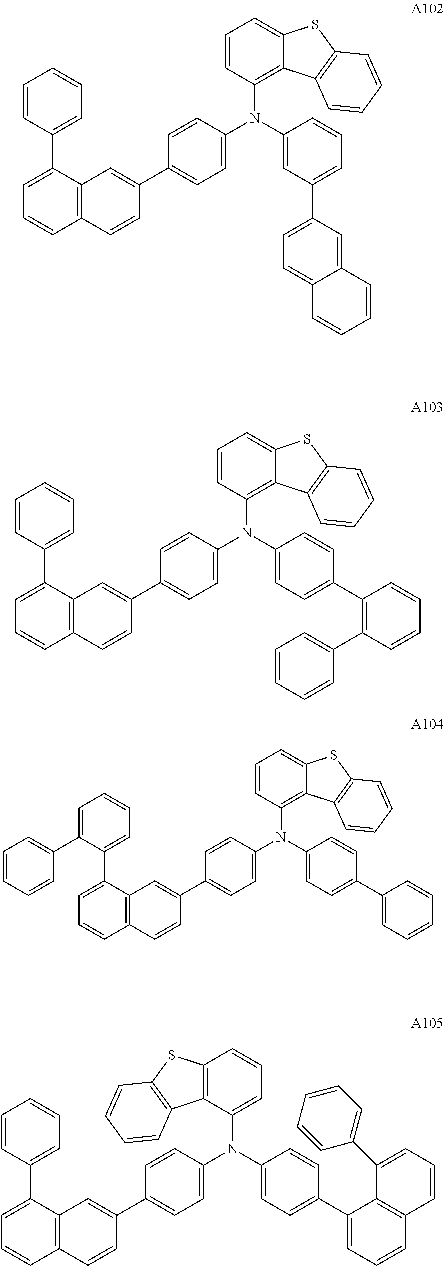 [SMILES: c1ccc(-c2cccc3ccc(-c4ccc(N(c5cccc(-c6ccc7ccccc7c6)c5)c5cccc6sc7ccccc7c56)cc4)cc23)cc1 ; c1ccc(-c2ccccc2-c2ccc(N(c3ccc(-c4ccc5cccc(-c6ccccc6)c5c4)cc3)c3cccc4sc5ccccc5c34)cc2)cc1 ; c1ccc(-c2ccc(N(c3ccc(-c4ccc5cccc(-c6ccccc6-c6ccccc6)c5c4)cc3)c3cccc4sc5ccccc5c34)cc2)cc1 ; c1ccc(-c2cccc3ccc(-c4ccc(N(c5ccc(-c6cccc7cccc(-c8ccccc8)c67)cc5)c5cccc6sc7ccccc7c56)cc4)cc23)cc1]

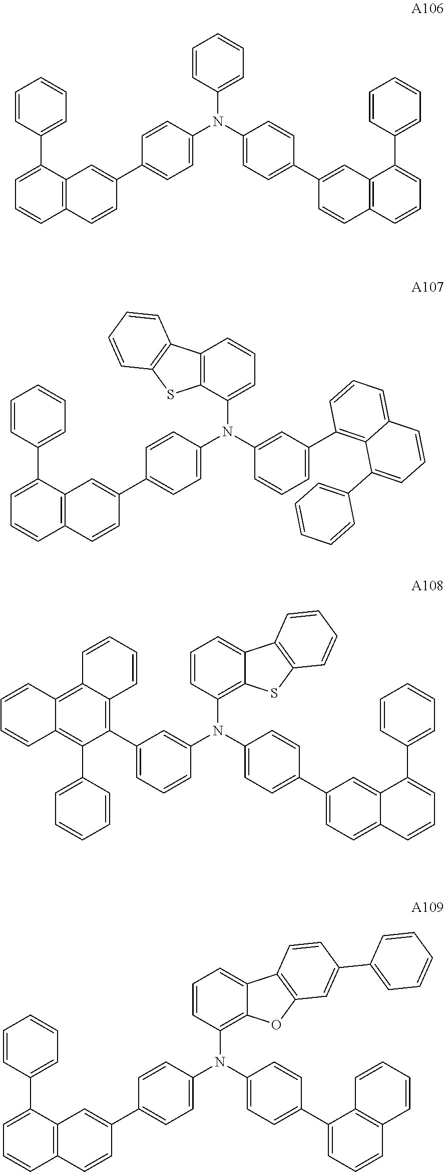 [SMILES: c1ccc(-c2cccc3ccc(-c4ccc(N(c5ccccc5)c5ccc(-c6ccc7cccc(-c8ccccc8)c7c6)cc5)cc4)cc23)cc1 ; c1ccc(-c2cccc3ccc(-c4ccc(N(c5cccc(-c6cccc7cccc(-c8ccccc8)c67)c5)c5cccc6c5sc5ccccc56)cc4)cc23)cc1 ; c1ccc(-c2cccc3ccc(-c4ccc(N(c5cccc(-c6c(-c7ccccc7)c7ccccc7c7ccccc67)c5)c5cccc6c5sc5ccccc56)cc4)cc23)cc1 ; c1ccc(-c2ccc3c(c2)oc2c(N(c4ccc(-c5ccc6cccc(-c7ccccc7)c6c5)cc4)c4ccc(-c5cccc6ccccc56)cc4)cccc23)cc1]

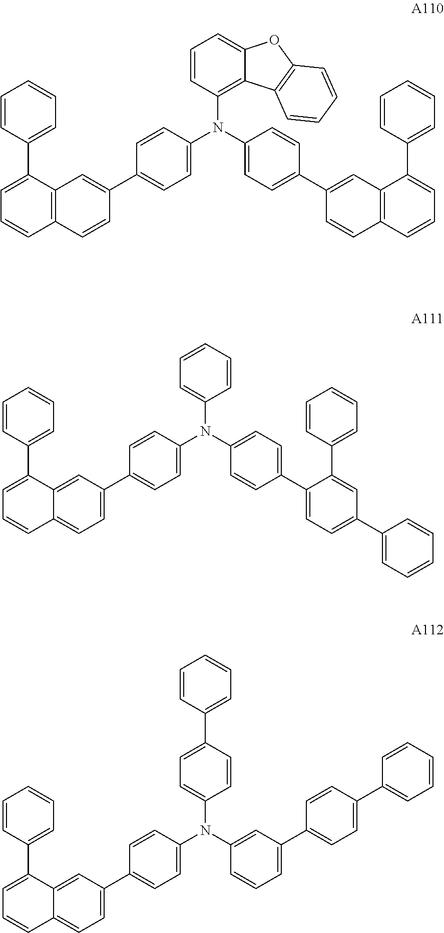 [SMILES: c1ccc(-c2cccc3ccc(-c4ccc(N(c5ccc(-c6ccc7cccc(-c8ccccc8)c7c6)cc5)c5cccc6oc7ccccc7c56)cc4)cc23)cc1 ; c1ccc(-c2ccc(-c3ccc(N(c4ccccc4)c4ccc(-c5ccc6cccc(-c7ccccc7)c6c5)cc4)cc3)c(-c3ccccc3)c2)cc1 ; c1ccc(-c2ccc(-c3cccc(N(c4ccc(-c5ccccc5)cc4)c4ccc(-c5ccc6cccc(-c7ccccc7)c6c5)cc4)c3)cc2)cc1]

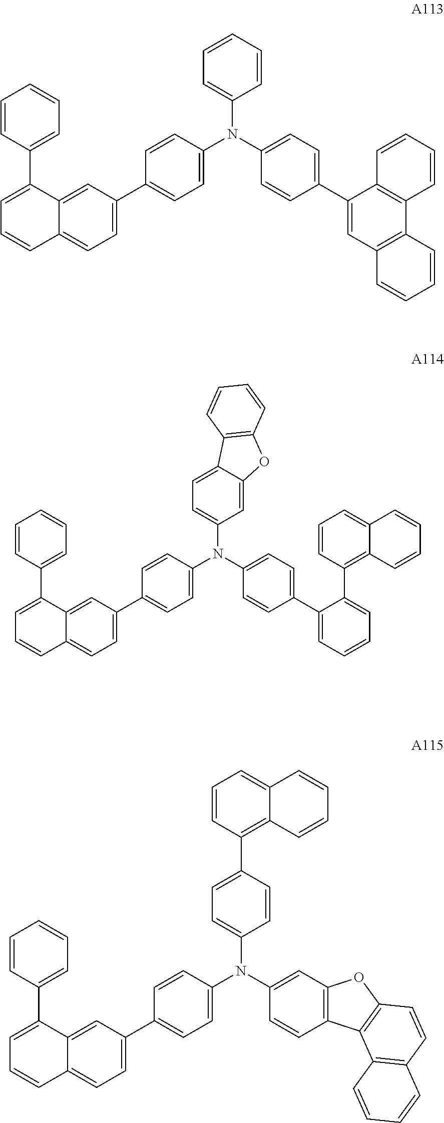 [SMILES: c1ccc(-c2cccc3ccc(-c4ccc(N(c5ccccc5)c5ccc(-c6cc7ccccc7c7ccccc67)cc5)cc4)cc23)cc1 ; c1ccc(-c2cccc3ccc(-c4ccc(N(c5ccc(-c6ccccc6-c6cccc7ccccc67)cc5)c5ccc6c(c5)oc5ccccc56)cc4)cc23)cc1 ; c1ccc(-c2cccc3ccc(-c4ccc(N(c5ccc(-c6cccc7ccccc67)cc5)c5ccc6c(c5)oc5ccc7ccccc7c56)cc4)cc23)cc1]

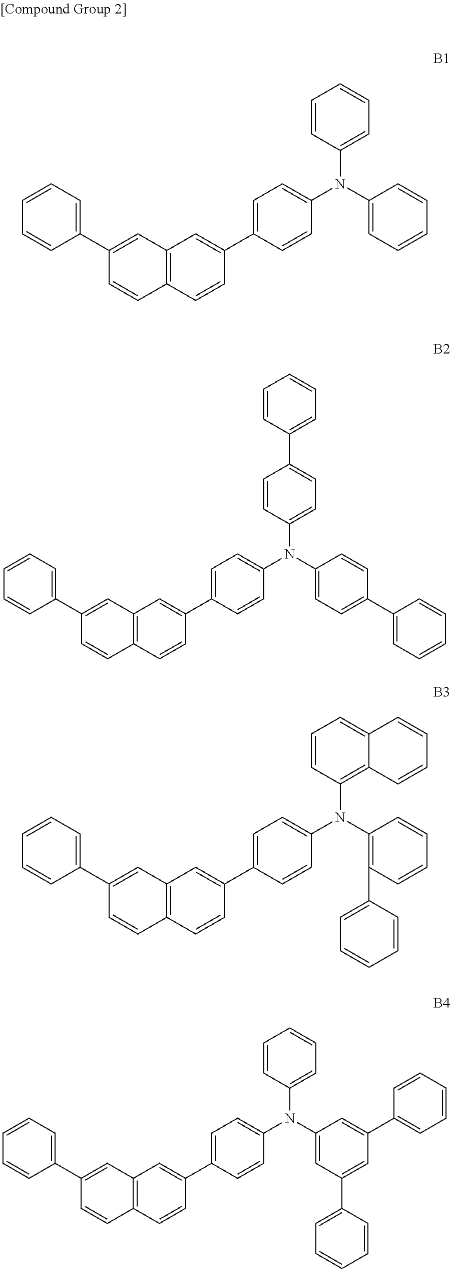 [SMILES: c1ccc(-c2ccc3ccc(-c4ccc(N(c5ccccc5)c5ccccc5)cc4)cc3c2)cc1 ; c1ccc(-c2ccc(N(c3ccc(-c4ccccc4)cc3)c3ccc(-c4ccc5ccc(-c6ccccc6)cc5c4)cc3)cc2)cc1 ; c1ccc(-c2ccc3ccc(-c4ccc(N(c5ccccc5-c5ccccc5)c5cccc6ccccc56)cc4)cc3c2)cc1 ; c1ccc(-c2cc(-c3ccccc3)cc(N(c3ccccc3)c3ccc(-c4ccc5ccc(-c6ccccc6)cc5c4)cc3)c2)cc1]

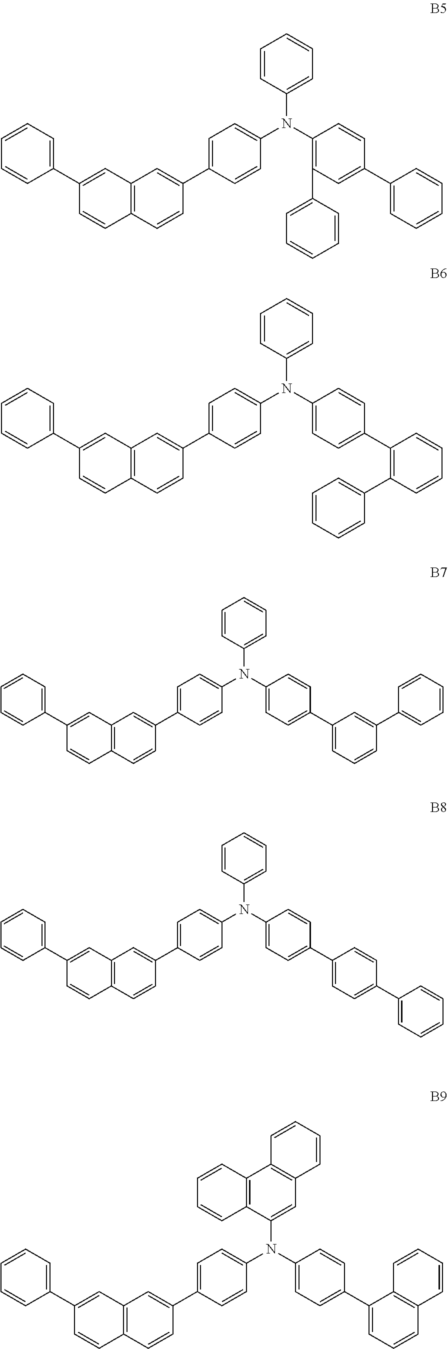 [SMILES: c1ccc(-c2ccc(N(c3ccccc3)c3ccc(-c4ccc5ccc(-c6ccccc6)cc5c4)cc3)c(-c3ccccc3)c2)cc1 ; c1ccc(-c2ccc3ccc(-c4ccc(N(c5ccccc5)c5ccc(-c6ccccc6-c6ccccc6)cc5)cc4)cc3c2)cc1 ; c1ccc(-c2cccc(-c3ccc(N(c4ccccc4)c4ccc(-c5ccc6ccc(-c7ccccc7)cc6c5)cc4)cc3)c2)cc1 ; c1ccc(-c2ccc(-c3ccc(N(c4ccccc4)c4ccc(-c5ccc6ccc(-c7ccccc7)cc6c5)cc4)cc3)cc2)cc1 ; c1ccc(-c2ccc3ccc(-c4ccc(N(c5ccc(-c6cccc7ccccc67)cc5)c5cc6ccccc6c6ccccc56)cc4)cc3c2)cc1]

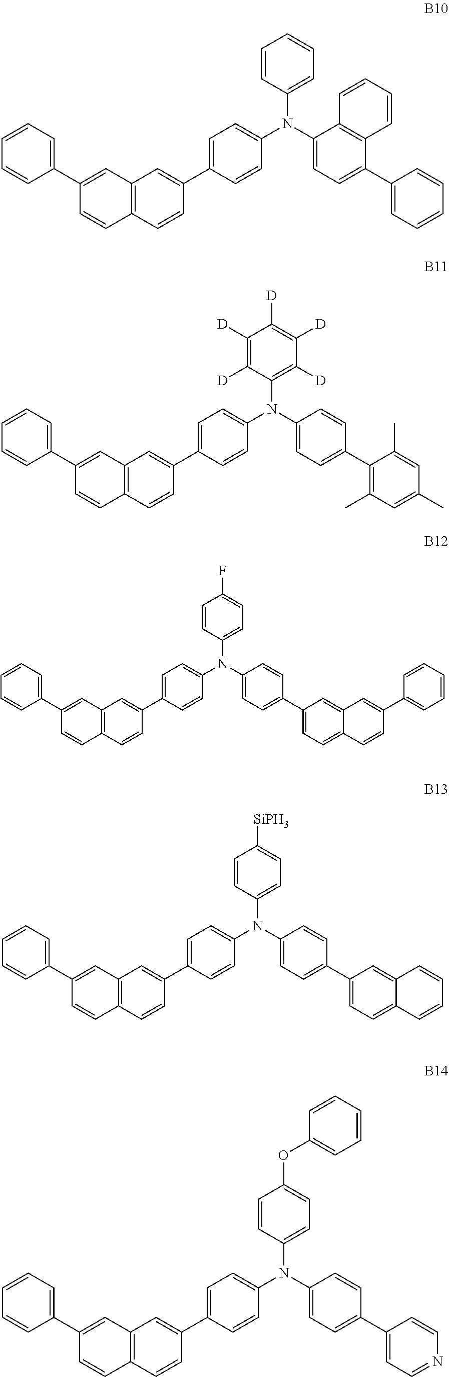

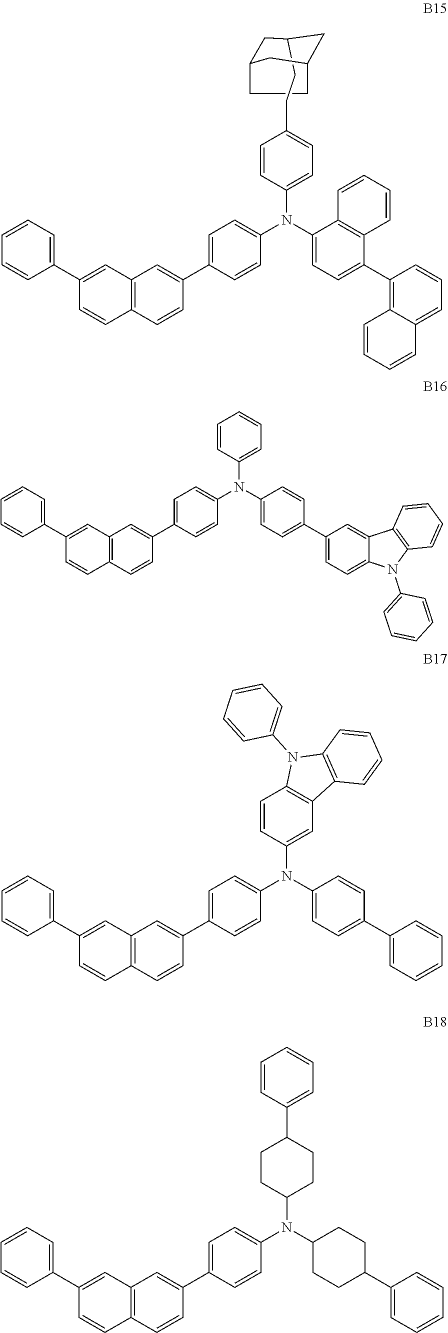 [SMILES: c1ccc(-c2ccc3ccc(-c4ccc(N(c5ccc(CCC6CC7CCC(C6)C7)cc5)c5ccc(-c6cccc7ccccc67)c6ccccc56)cc4)cc3c2)cc1 ; c1ccc(-c2ccc3ccc(-c4ccc(N(c5ccccc5)c5ccc(-c6ccc7c(c6)c6ccccc6n7-c6ccccc6)cc5)cc4)cc3c2)cc1 ; c1ccc(-c2ccc(N(c3ccc(-c4ccc5ccc(-c6ccccc6)cc5c4)cc3)c3ccc4c(c3)c3ccccc3n4-c3ccccc3)cc2)cc1 ; c1ccc(-c2ccc3ccc(-c4ccc(N(C5CCC(c6ccccc6)CC5)C5CCC(c6ccccc6)CC5)cc4)cc3c2)cc1]

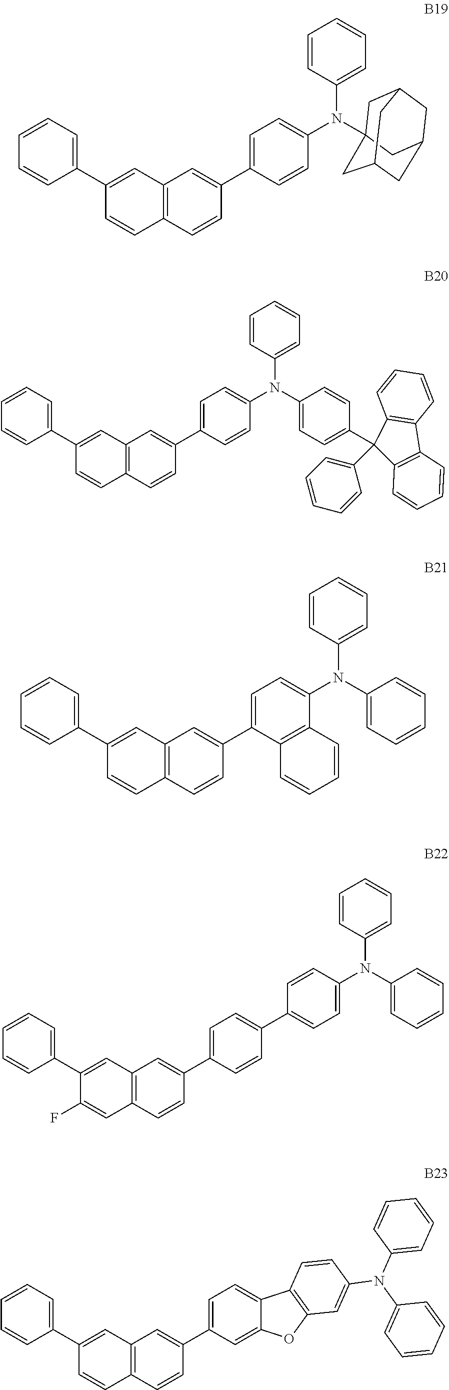 [SMILES: c1ccc(-c2ccc3ccc(-c4ccc(N(c5ccccc5)C56CC7CC(CC(C7)C5)C6)cc4)cc3c2)cc1 ; c1ccc(-c2ccc3ccc(-c4ccc(N(c5ccccc5)c5ccc(C6(c7ccccc7)c7ccccc7-c7ccccc76)cc5)cc4)cc3c2)cc1 ; c1ccc(-c2ccc3ccc(-c4ccc(N(c5ccccc5)c5ccccc5)c5ccccc45)cc3c2)cc1 ; Fc1cc2ccc(-c3ccc(-c4ccc(N(c5ccccc5)c5ccccc5)cc4)cc3)cc2cc1-c1ccccc1 ; c1ccc(-c2ccc3ccc(-c4ccc5c(c4)oc4cc(N(c6ccccc6)c6ccccc6)ccc45)cc3c2)cc1]

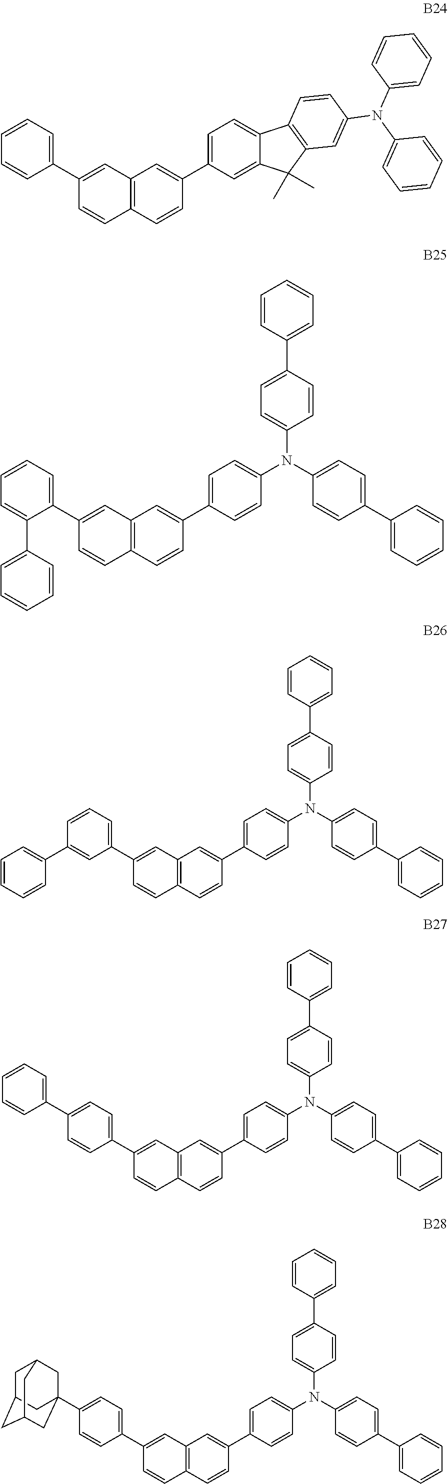 [SMILES: CC1(C)c2cc(-c3ccc4ccc(-c5ccccc5)cc4c3)ccc2-c2ccc(N(c3ccccc3)c3ccccc3)cc21 ; c1ccc(-c2ccc(N(c3ccc(-c4ccccc4)cc3)c3ccc(-c4ccc5ccc(-c6ccccc6-c6ccccc6)cc5c4)cc3)cc2)cc1 ; c1ccc(-c2ccc(N(c3ccc(-c4ccccc4)cc3)c3ccc(-c4ccc5ccc(-c6cccc(-c7ccccc7)c6)cc5c4)cc3)cc2)cc1 ; c1ccc(-c2ccc(-c3ccc4ccc(-c5ccc(N(c6ccc(-c7ccccc7)cc6)c6ccc(-c7ccccc7)cc6)cc5)cc4c3)cc2)cc1 ; c1ccc(-c2ccc(N(c3ccc(-c4ccccc4)cc3)c3ccc(-c4ccc5ccc(-c6ccc(C78CC9CC(CC(C9)C7)C8)cc6)cc5c4)cc3)cc2)cc1]

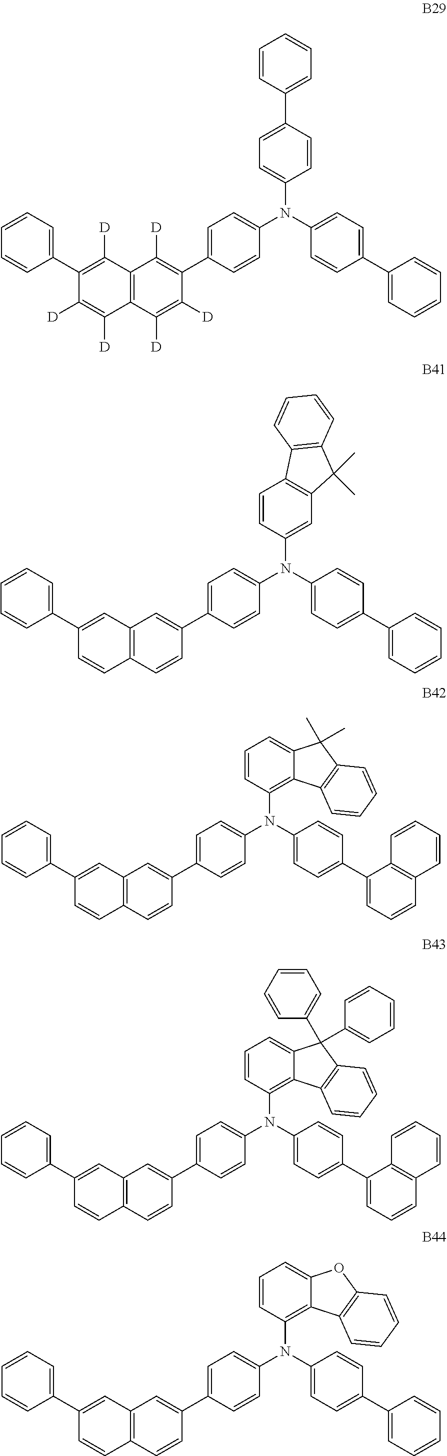 [SMILES: [2H]c1c(-c2ccccc2)c([2H])c2c([2H])c(-c3ccc(N(c4ccc(-c5ccccc5)cc4)c4ccc(-c5ccccc5)cc4)cc3)c([2H])c([2H])c2c1[2H] ; CC1(C)c2ccccc2-c2ccc(N(c3ccc(-c4ccccc4)cc3)c3ccc(-c4ccc5ccc(-c6ccccc6)cc5c4)cc3)cc21 ; CC1(C)c2ccccc2-c2c(N(c3ccc(-c4ccc5ccc(-c6ccccc6)cc5c4)cc3)c3ccc(-c4cccc5ccccc45)cc3)cccc21 ; c1ccc(-c2ccc3ccc(-c4ccc(N(c5ccc(-c6cccc7ccccc67)cc5)c5cccc6c5-c5ccccc5C6(c5ccccc5)c5ccccc5)cc4)cc3c2)cc1 ; c1ccc(-c2ccc(N(c3ccc(-c4ccc5ccc(-c6ccccc6)cc5c4)cc3)c3cccc4oc5ccccc5c34)cc2)cc1]

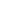

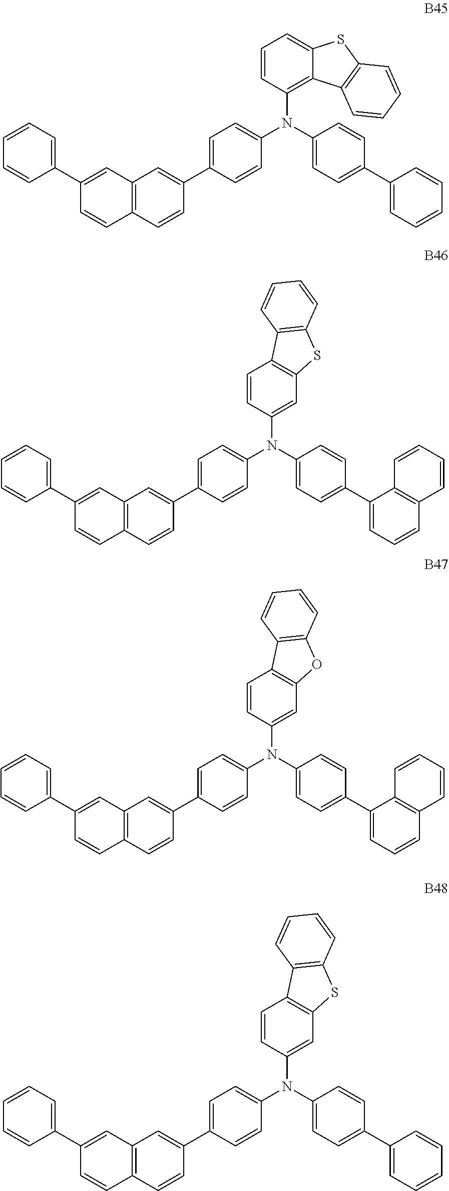 [SMILES: c1ccc(-c2ccc(N(c3ccc(-c4ccc5ccc(-c6ccccc6)cc5c4)cc3)c3cccc4sc5ccccc5c34)cc2)cc1 ; c1ccc(-c2ccc3ccc(-c4ccc(N(c5ccc(-c6cccc7ccccc67)cc5)c5ccc6c(c5)sc5ccccc56)cc4)cc3c2)cc1 ; c1ccc(-c2ccc3ccc(-c4ccc(N(c5ccc(-c6cccc7ccccc67)cc5)c5ccc6c(c5)oc5ccccc56)cc4)cc3c2)cc1 ; c1ccc(-c2ccc(N(c3ccc(-c4ccc5ccc(-c6ccccc6)cc5c4)cc3)c3ccc4c(c3)sc3ccccc34)cc2)cc1]

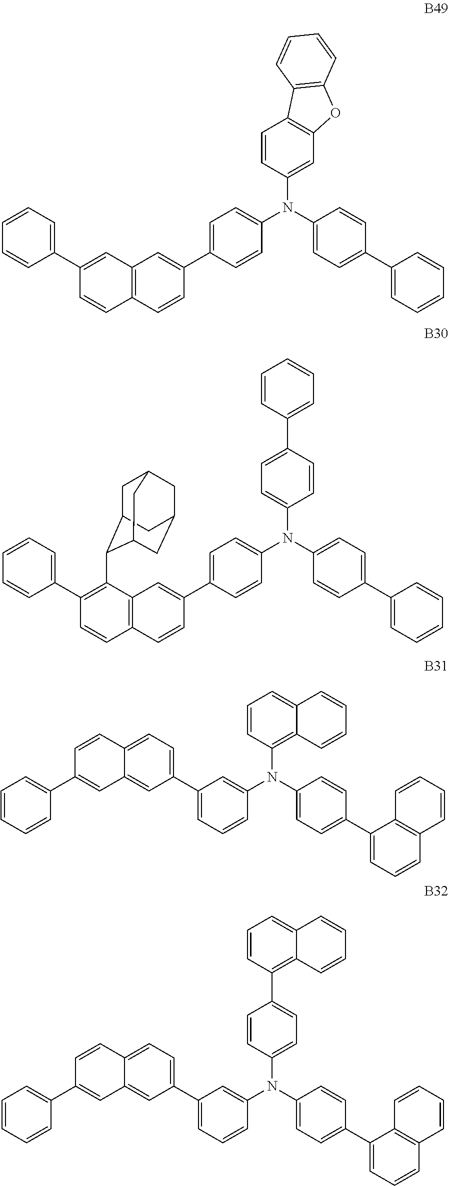 [SMILES: c1ccc(-c2ccc(N(c3ccc(-c4ccc5ccc(-c6ccccc6)cc5c4)cc3)c3ccc4c(c3)oc3ccccc34)cc2)cc1 ; c1ccc(-c2ccc(N(c3ccc(-c4ccccc4)cc3)c3ccc(-c4ccc5ccc(-c6ccccc6)c(C6C7CC8CC(C7)CC6C8)c5c4)cc3)cc2)cc1 ; c1ccc(-c2ccc3ccc(-c4cccc(N(c5ccc(-c6cccc7ccccc67)cc5)c5cccc6ccccc56)c4)cc3c2)cc1 ; c1ccc(-c2ccc3ccc(-c4cccc(N(c5ccc(-c6cccc7ccccc67)cc5)c5ccc(-c6cccc7ccccc67)cc5)c4)cc3c2)cc1]

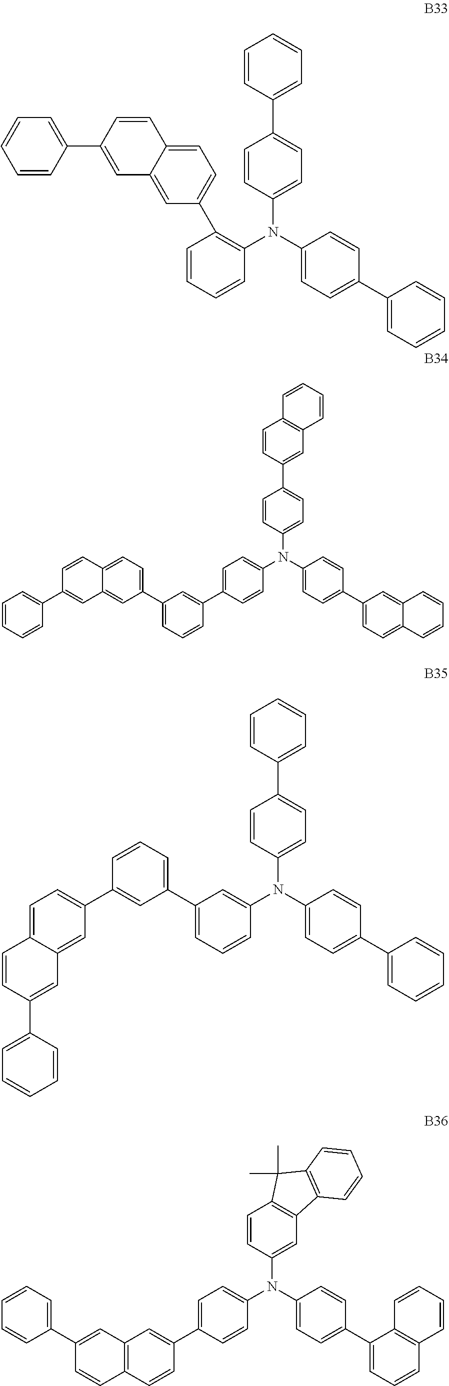 [SMILES: c1ccc(-c2ccc(N(c3ccc(-c4ccccc4)cc3)c3ccccc3-c3ccc4ccc(-c5ccccc5)cc4c3)cc2)cc1 ; c1ccc(-c2ccc3ccc(-c4cccc(-c5ccc(N(c6ccc(-c7ccc8ccccc8c7)cc6)c6ccc(-c7ccc8ccccc8c7)cc6)cc5)c4)cc3c2)cc1 ; c1ccc(-c2ccc(N(c3ccc(-c4ccccc4)cc3)c3cccc(-c4cccc(-c5ccc6ccc(-c7ccccc7)cc6c5)c4)c3)cc2)cc1 ; CC1(C)c2ccccc2-c2cc(N(c3ccc(-c4ccc5ccc(-c6ccccc6)cc5c4)cc3)c3ccc(-c4cccc5ccccc45)cc3)ccc21]

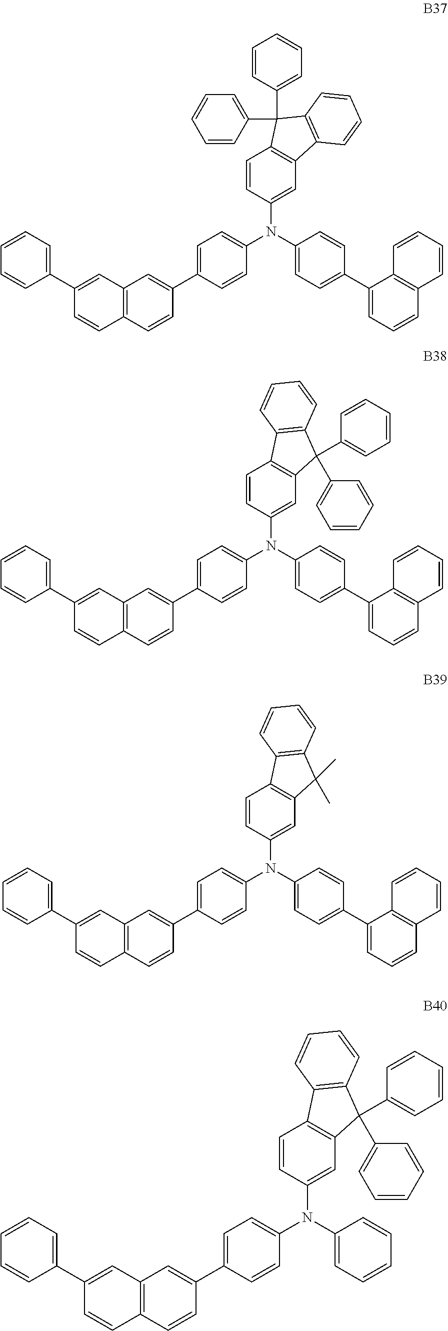 [SMILES: c1ccc(-c2ccc3ccc(-c4ccc(N(c5ccc(-c6cccc7ccccc67)cc5)c5ccc6c(c5)-c5ccccc5C6(c5ccccc5)c5ccccc5)cc4)cc3c2)cc1 ; c1ccc(-c2ccc3ccc(-c4ccc(N(c5ccc(-c6cccc7ccccc67)cc5)c5ccc6c(c5)C(c5ccccc5)(c5ccccc5)c5ccccc5-6)cc4)cc3c2)cc1 ; CC1(C)c2ccccc2-c2ccc(N(c3ccc(-c4ccc5ccc(-c6ccccc6)cc5c4)cc3)c3ccc(-c4cccc5ccccc45)cc3)cc21 ; c1ccc(-c2ccc3ccc(-c4ccc(N(c5ccccc5)c5ccc6c(c5)C(c5ccccc5)(c5ccccc5)c5ccccc5-6)cc4)cc3c2)cc1]

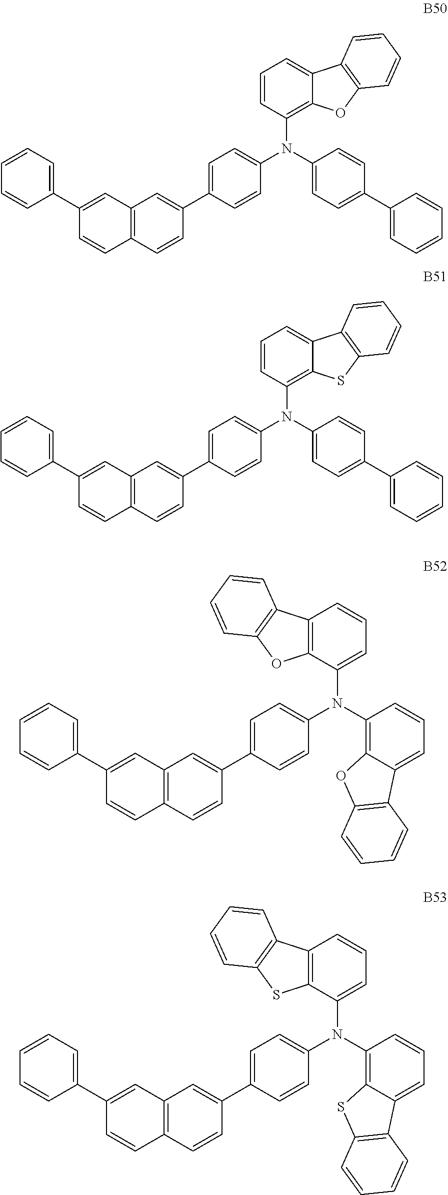 [SMILES: c1ccc(-c2ccc(N(c3ccc(-c4ccc5ccc(-c6ccccc6)cc5c4)cc3)c3cccc4c3oc3ccccc34)cc2)cc1 ; c1ccc(-c2ccc(N(c3ccc(-c4ccc5ccc(-c6ccccc6)cc5c4)cc3)c3cccc4c3sc3ccccc34)cc2)cc1 ; c1ccc(-c2ccc3ccc(-c4ccc(N(c5cccc6c5oc5ccccc56)c5cccc6c5oc5ccccc56)cc4)cc3c2)cc1 ; c1ccc(-c2ccc3ccc(-c4ccc(N(c5cccc6c5sc5ccccc56)c5cccc6c5sc5ccccc56)cc4)cc3c2)cc1]

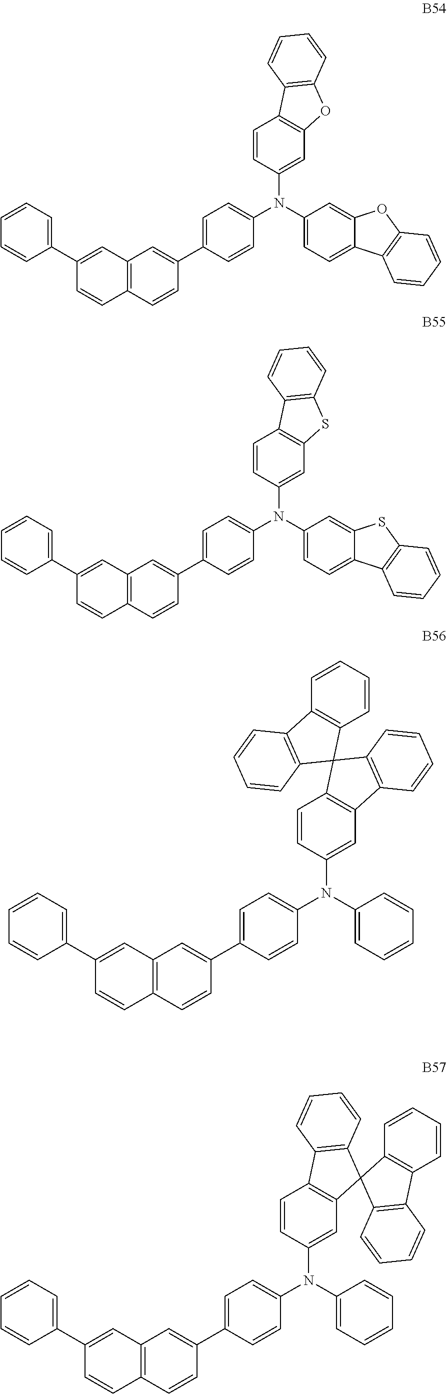 [SMILES: c1ccc(-c2ccc3ccc(-c4ccc(N(c5ccc6c(c5)oc5ccccc56)c5ccc6c(c5)oc5ccccc56)cc4)cc3c2)cc1 ; c1ccc(-c2ccc3ccc(-c4ccc(N(c5ccc6c(c5)sc5ccccc56)c5ccc6c(c5)sc5ccccc56)cc4)cc3c2)cc1 ; c1ccc(-c2ccc3ccc(-c4ccc(N(c5ccccc5)c5ccc6c(c5)-c5ccccc5C65c6ccccc6-c6ccccc65)cc4)cc3c2)cc1 ; c1ccc(-c2ccc3ccc(-c4ccc(N(c5ccccc5)c5ccc6c(c5)C5(c7ccccc7-c7ccccc75)c5ccccc5-6)cc4)cc3c2)cc1]

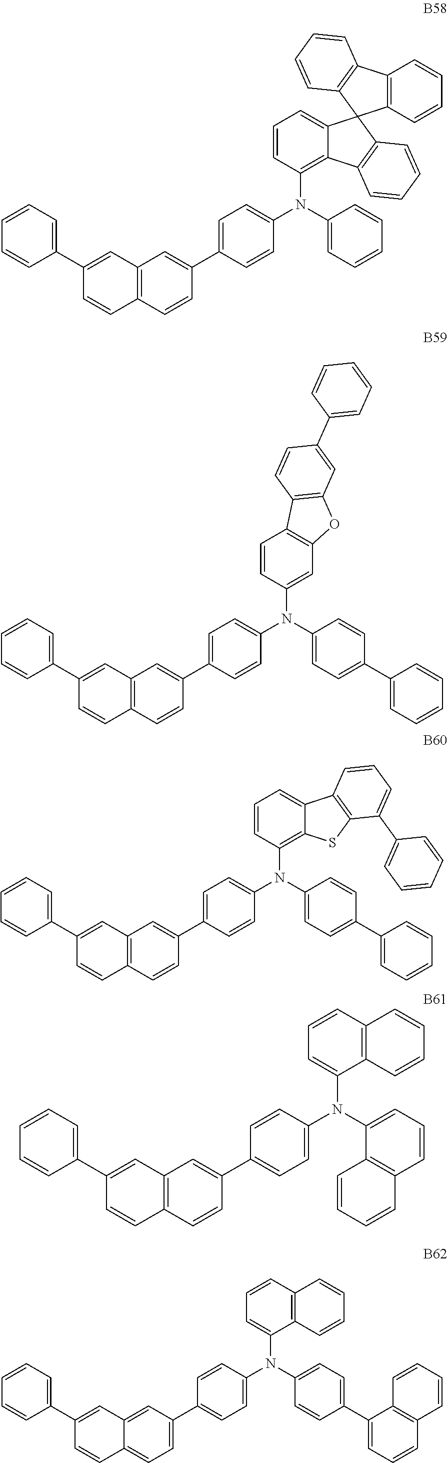

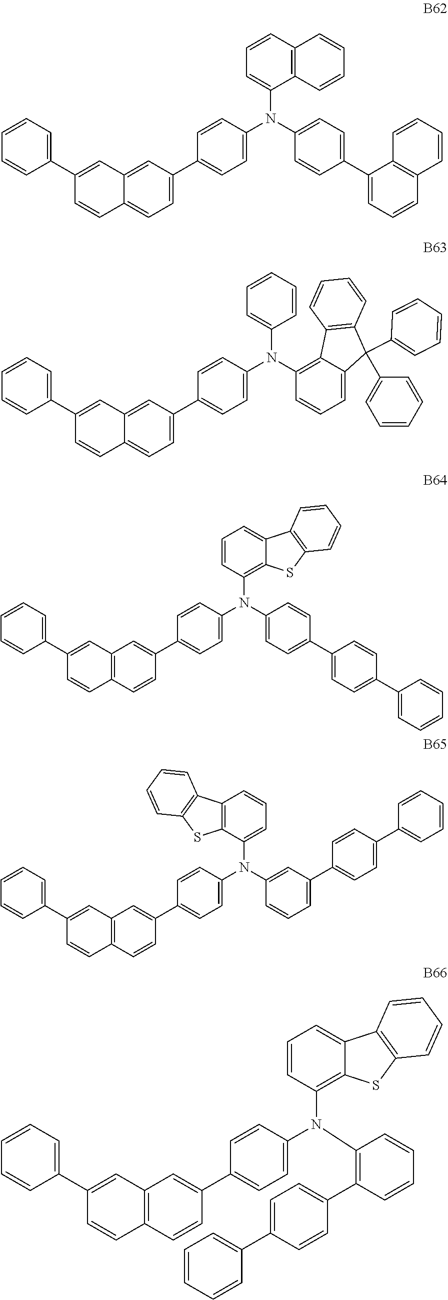 [SMILES: c1ccc(-c2ccc3ccc(-c4ccc(N(c5ccc(-c6cccc7ccccc67)cc5)c5cccc6ccccc56)cc4)cc3c2)cc1 ; c1ccc(-c2ccc3ccc(-c4ccc(N(c5ccccc5)c5cccc6c5-c5ccccc5C6(c5ccccc5)c5ccccc5)cc4)cc3c2)cc1 ; c1ccc(-c2ccc(-c3ccc(N(c4ccc(-c5ccc6ccc(-c7ccccc7)cc6c5)cc4)c4cccc5c4sc4ccccc45)cc3)cc2)cc1 ; c1ccc(-c2ccc(-c3cccc(N(c4ccc(-c5ccc6ccc(-c7ccccc7)cc6c5)cc4)c4cccc5c4sc4ccccc45)c3)cc2)cc1 ; c1ccc(-c2ccc(-c3ccccc3N(c3ccc(-c4ccc5ccc(-c6ccccc6)cc5c4)cc3)c3cccc4c3sc3ccccc34)cc2)cc1]

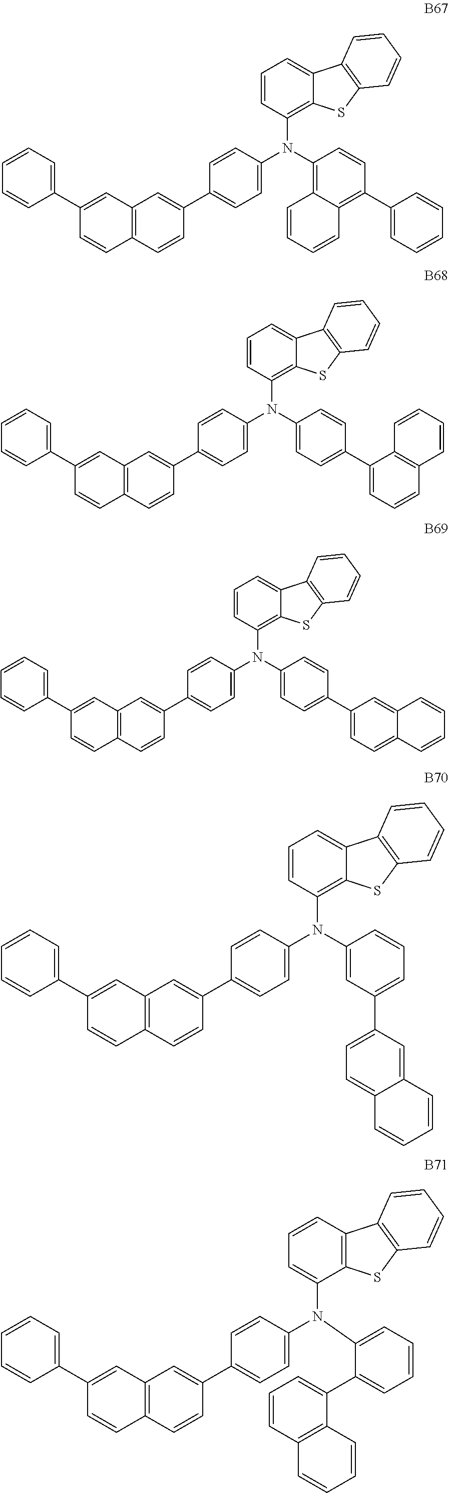 [SMILES: c1ccc(-c2ccc3ccc(-c4ccc(N(c5ccc(-c6ccccc6)c6ccccc56)c5cccc6c5sc5ccccc56)cc4)cc3c2)cc1 ; c1ccc(-c2ccc3ccc(-c4ccc(N(c5ccc(-c6cccc7ccccc67)cc5)c5cccc6c5sc5ccccc56)cc4)cc3c2)cc1 ; c1ccc(-c2ccc3ccc(-c4ccc(N(c5ccc(-c6ccc7ccccc7c6)cc5)c5cccc6c5sc5ccccc56)cc4)cc3c2)cc1 ; c1ccc(-c2ccc3ccc(-c4ccc(N(c5cccc(-c6ccc7ccccc7c6)c5)c5cccc6c5sc5ccccc56)cc4)cc3c2)cc1 ; c1ccc(-c2ccc3ccc(-c4ccc(N(c5ccccc5-c5cccc6ccccc56)c5cccc6c5sc5ccccc56)cc4)cc3c2)cc1]

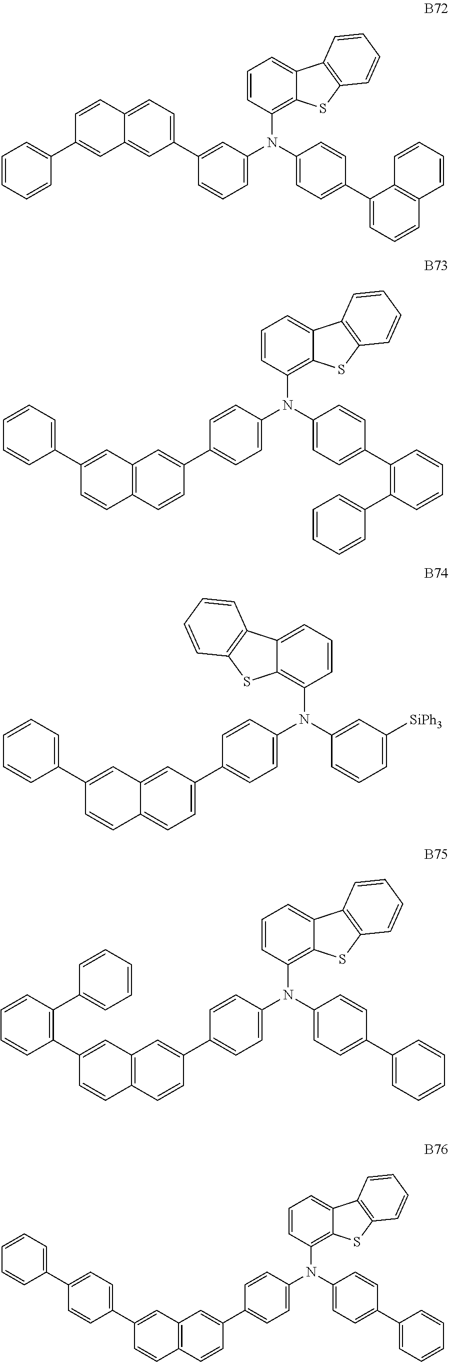

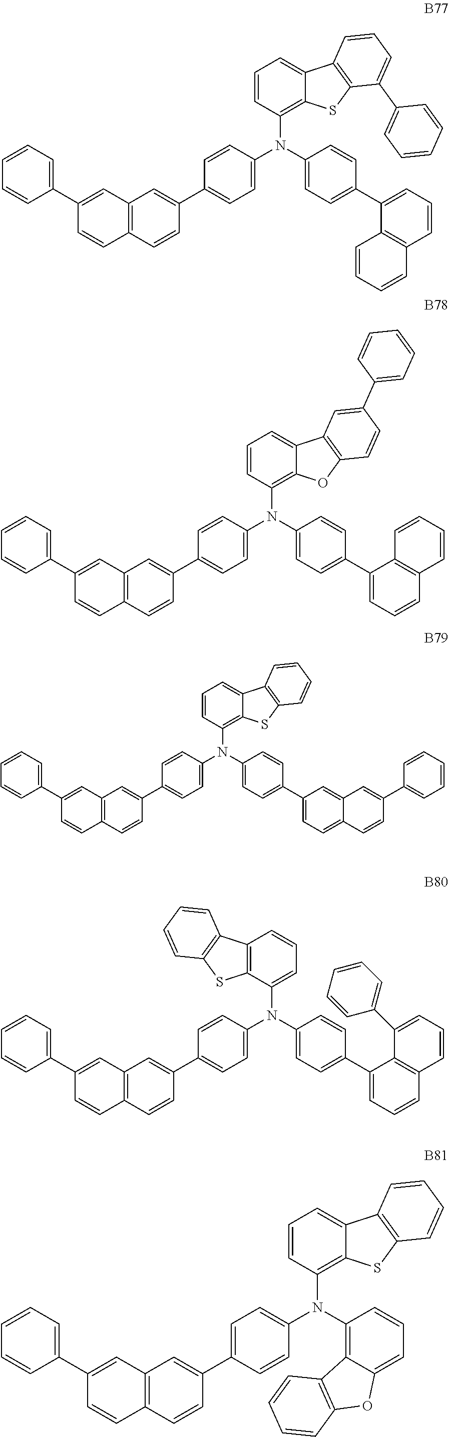 [SMILES: c1ccc(-c2ccc3ccc(-c4ccc(N(c5ccc(-c6cccc7ccccc67)cc5)c5cccc6c5sc5c(-c7ccccc7)cccc56)cc4)cc3c2)cc1 ; c1ccc(-c2ccc3ccc(-c4ccc(N(c5ccc(-c6cccc7ccccc67)cc5)c5cccc6c5oc5ccc(-c7ccccc7)cc56)cc4)cc3c2)cc1 ; c1ccc(-c2ccc3ccc(-c4ccc(N(c5ccc(-c6ccc7ccc(-c8ccccc8)cc7c6)cc5)c5cccc6c5sc5ccccc56)cc4)cc3c2)cc1 ; c1ccc(-c2ccc3ccc(-c4ccc(N(c5ccc(-c6cccc7cccc(-c8ccccc8)c67)cc5)c5cccc6c5sc5ccccc56)cc4)cc3c2)cc1 ; c1ccc(-c2ccc3ccc(-c4ccc(N(c5cccc6c5sc5ccccc56)c5cccc6oc7ccccc7c56)cc4)cc3c2)cc1]

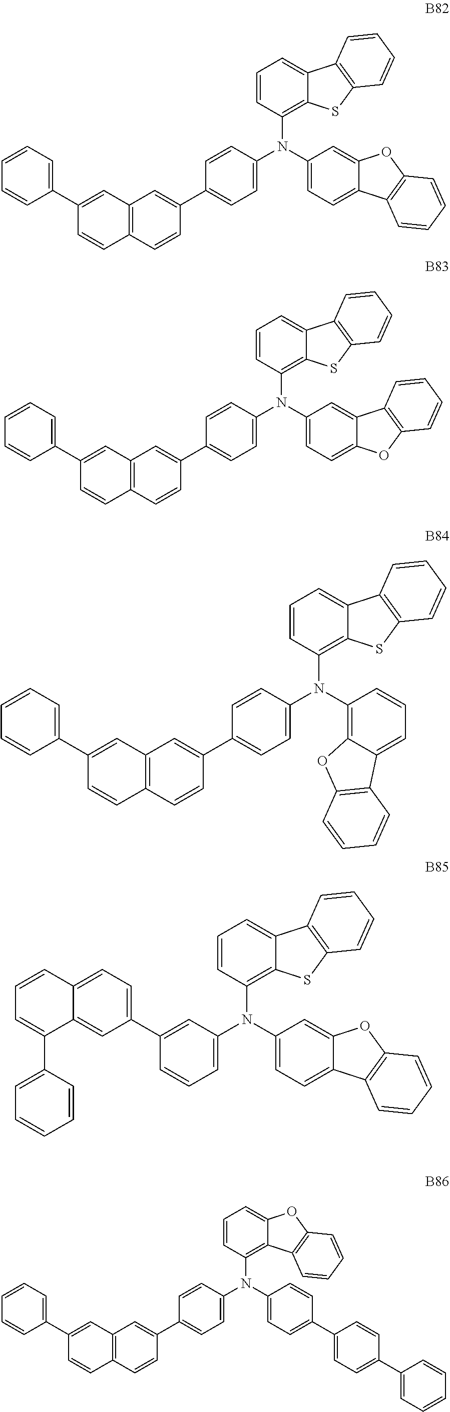 [SMILES: c1ccc(-c2ccc3ccc(-c4ccc(N(c5ccc6c(c5)oc5ccccc56)c5cccc6c5sc5ccccc56)cc4)cc3c2)cc1 ; c1ccc(-c2ccc3ccc(-c4ccc(N(c5ccc6oc7ccccc7c6c5)c5cccc6c5sc5ccccc56)cc4)cc3c2)cc1 ; c1ccc(-c2ccc3ccc(-c4ccc(N(c5cccc6c5oc5ccccc56)c5cccc6c5sc5ccccc56)cc4)cc3c2)cc1 ; c1ccc(-c2cccc3ccc(-c4cccc(N(c5ccc6c(c5)oc5ccccc56)c5cccc6c5sc5ccccc56)c4)cc23)cc1 ; c1ccc(-c2ccc(-c3ccc(N(c4ccc(-c5ccc6ccc(-c7ccccc7)cc6c5)cc4)c4cccc5oc6ccccc6c45)cc3)cc2)cc1]

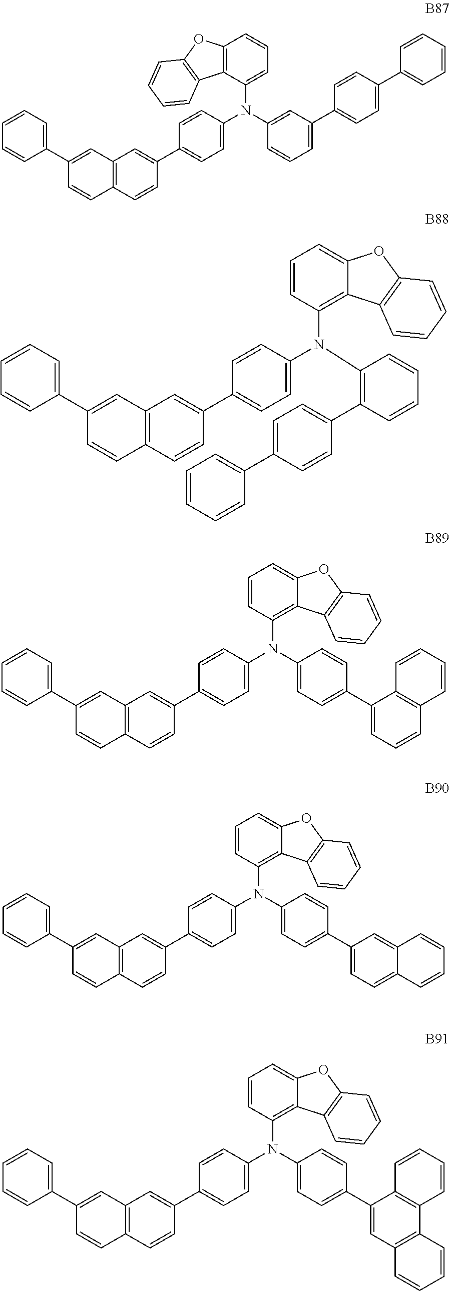 [SMILES: c1ccc(-c2ccc(-c3cccc(N(c4ccc(-c5ccc6ccc(-c7ccccc7)cc6c5)cc4)c4cccc5oc6ccccc6c45)c3)cc2)cc1 ; c1ccc(-c2ccc(-c3ccccc3N(c3ccc(-c4ccc5ccc(-c6ccccc6)cc5c4)cc3)c3cccc4oc5ccccc5c34)cc2)cc1 ; c1ccc(-c2ccc3ccc(-c4ccc(N(c5ccc(-c6cccc7ccccc67)cc5)c5cccc6oc7ccccc7c56)cc4)cc3c2)cc1 ; c1ccc(-c2ccc3ccc(-c4ccc(N(c5ccc(-c6ccc7ccccc7c6)cc5)c5cccc6oc7ccccc7c56)cc4)cc3c2)cc1 ; c1ccc(-c2ccc3ccc(-c4ccc(N(c5ccc(-c6cc7ccccc7c7ccccc67)cc5)c5cccc6oc7ccccc7c56)cc4)cc3c2)cc1]

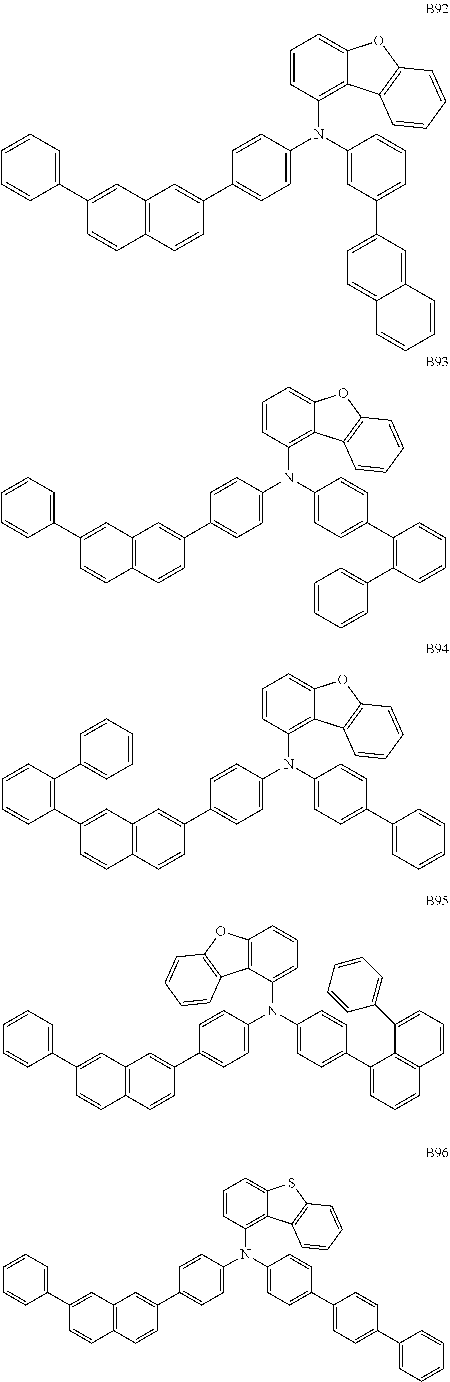 [SMILES: c1ccc(-c2ccc3ccc(-c4ccc(N(c5cccc(-c6ccc7ccccc7c6)c5)c5cccc6oc7ccccc7c56)cc4)cc3c2)cc1 ; c1ccc(-c2ccc3ccc(-c4ccc(N(c5ccc(-c6ccccc6-c6ccccc6)cc5)c5cccc6oc7ccccc7c56)cc4)cc3c2)cc1 ; c1ccc(-c2ccc(N(c3ccc(-c4ccc5ccc(-c6ccccc6-c6ccccc6)cc5c4)cc3)c3cccc4oc5ccccc5c34)cc2)cc1 ; c1ccc(-c2ccc3ccc(-c4ccc(N(c5ccc(-c6cccc7cccc(-c8ccccc8)c67)cc5)c5cccc6oc7ccccc7c56)cc4)cc3c2)cc1 ; c1ccc(-c2ccc(-c3ccc(N(c4ccc(-c5ccc6ccc(-c7ccccc7)cc6c5)cc4)c4cccc5sc6ccccc6c45)cc3)cc2)cc1]

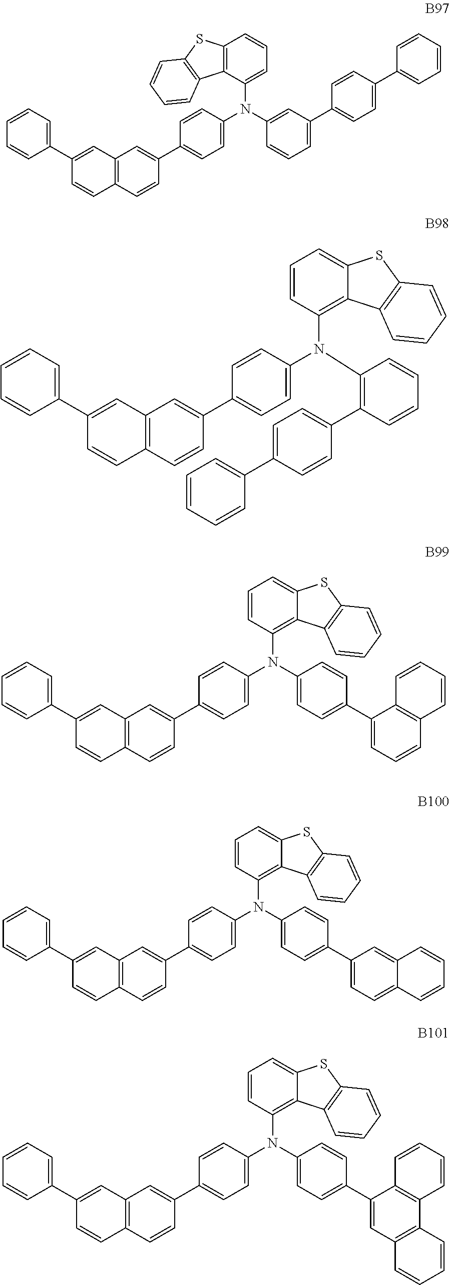 [SMILES: c1ccc(-c2ccc(-c3cccc(N(c4ccc(-c5ccc6ccc(-c7ccccc7)cc6c5)cc4)c4cccc5sc6ccccc6c45)c3)cc2)cc1 ; c1ccc(-c2ccc(-c3ccccc3N(c3ccc(-c4ccc5ccc(-c6ccccc6)cc5c4)cc3)c3cccc4sc5ccccc5c34)cc2)cc1 ; c1ccc(-c2ccc3ccc(-c4ccc(N(c5ccc(-c6cccc7ccccc67)cc5)c5cccc6sc7ccccc7c56)cc4)cc3c2)cc1 ; c1ccc(-c2ccc3ccc(-c4ccc(N(c5ccc(-c6ccc7ccccc7c6)cc5)c5cccc6sc7ccccc7c56)cc4)cc3c2)cc1 ; c1ccc(-c2ccc3ccc(-c4ccc(N(c5ccc(-c6cc7ccccc7c7ccccc67)cc5)c5cccc6sc7ccccc7c56)cc4)cc3c2)cc1]

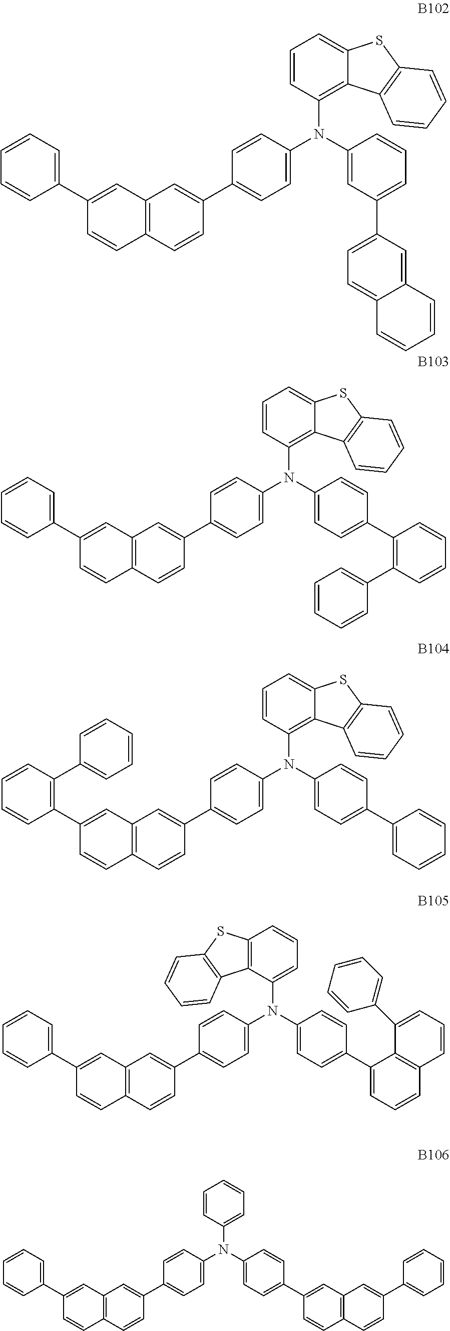 [SMILES: c1ccc(-c2ccc3ccc(-c4ccc(N(c5cccc(-c6ccc7ccccc7c6)c5)c5cccc6sc7ccccc7c56)cc4)cc3c2)cc1 ; c1ccc(-c2ccc3ccc(-c4ccc(N(c5ccc(-c6ccccc6-c6ccccc6)cc5)c5cccc6sc7ccccc7c56)cc4)cc3c2)cc1 ; c1ccc(-c2ccc(N(c3ccc(-c4ccc5ccc(-c6ccccc6-c6ccccc6)cc5c4)cc3)c3cccc4sc5ccccc5c34)cc2)cc1 ; c1ccc(-c2ccc3ccc(-c4ccc(N(c5ccc(-c6cccc7cccc(-c8ccccc8)c67)cc5)c5cccc6sc7ccccc7c56)cc4)cc3c2)cc1 ; c1ccc(-c2ccc3ccc(-c4ccc(N(c5ccccc5)c5ccc(-c6ccc7ccc(-c8ccccc8)cc7c6)cc5)cc4)cc3c2)cc1]

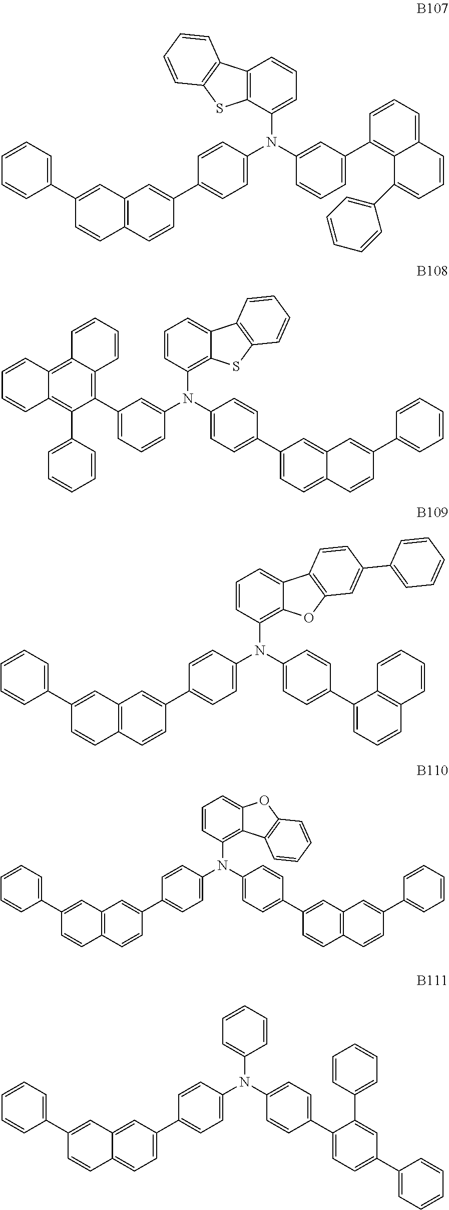 [SMILES: c1ccc(-c2ccc3ccc(-c4ccc(N(c5cccc(-c6cccc7cccc(-c8ccccc8)c67)c5)c5cccc6c5sc5ccccc56)cc4)cc3c2)cc1 ; c1ccc(-c2ccc3ccc(-c4ccc(N(c5cccc(-c6c(-c7ccccc7)c7ccccc7c7ccccc67)c5)c5cccc6c5sc5ccccc56)cc4)cc3c2)cc1 ; c1ccc(-c2ccc3ccc(-c4ccc(N(c5ccc(-c6cccc7ccccc67)cc5)c5cccc6c5oc5cc(-c7ccccc7)ccc56)cc4)cc3c2)cc1 ; c1ccc(-c2ccc3ccc(-c4ccc(N(c5ccc(-c6ccc7ccc(-c8ccccc8)cc7c6)cc5)c5cccc6oc7ccccc7c56)cc4)cc3c2)cc1 ; c1ccc(-c2ccc(-c3ccc(N(c4ccccc4)c4ccc(-c5ccc6ccc(-c7ccccc7)cc6c5)cc4)cc3)c(-c3ccccc3)c2)cc1]

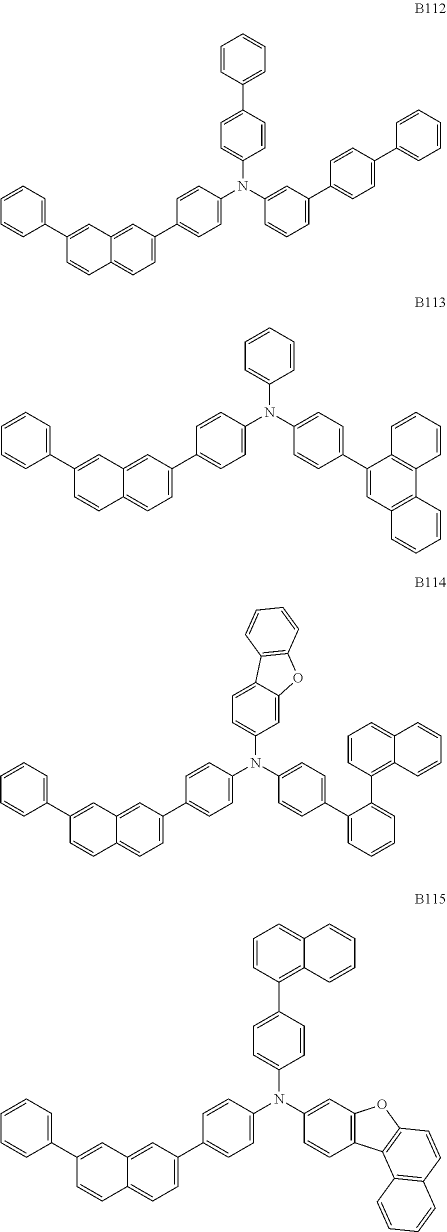 [SMILES: c1ccc(-c2ccc(-c3cccc(N(c4ccc(-c5ccccc5)cc4)c4ccc(-c5ccc6ccc(-c7ccccc7)cc6c5)cc4)c3)cc2)cc1 ; c1ccc(-c2ccc3ccc(-c4ccc(N(c5ccccc5)c5ccc(-c6cc7ccccc7c7ccccc67)cc5)cc4)cc3c2)cc1 ; c1ccc(-c2ccc3ccc(-c4ccc(N(c5ccc(-c6ccccc6-c6cccc7ccccc67)cc5)c5ccc6c(c5)oc5ccccc56)cc4)cc3c2)cc1 ; c1ccc(-c2ccc3ccc(-c4ccc(N(c5ccc(-c6cccc7ccccc67)cc5)c5ccc6c(c5)oc5ccc7ccccc7c56)cc4)cc3c2)cc1]

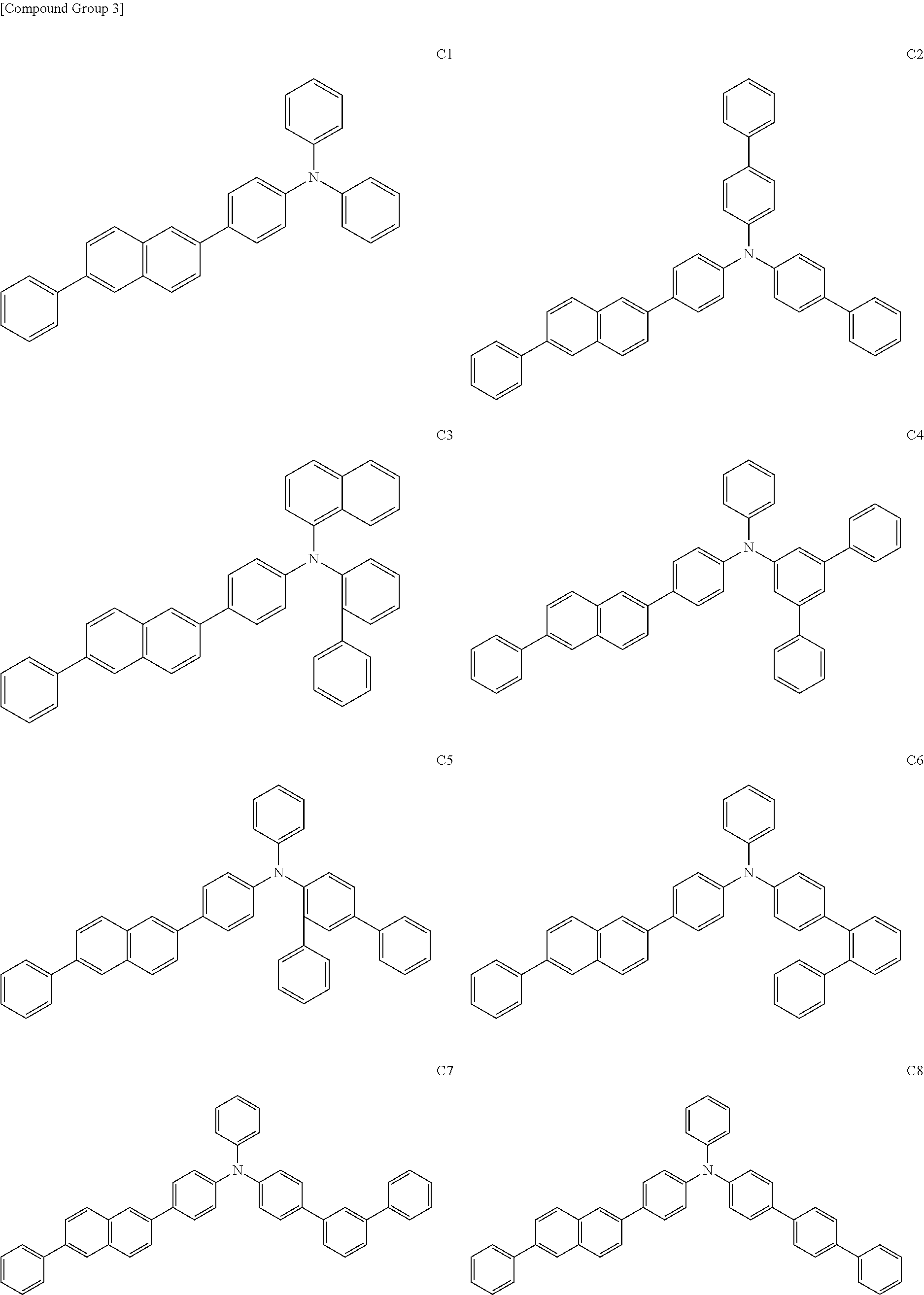

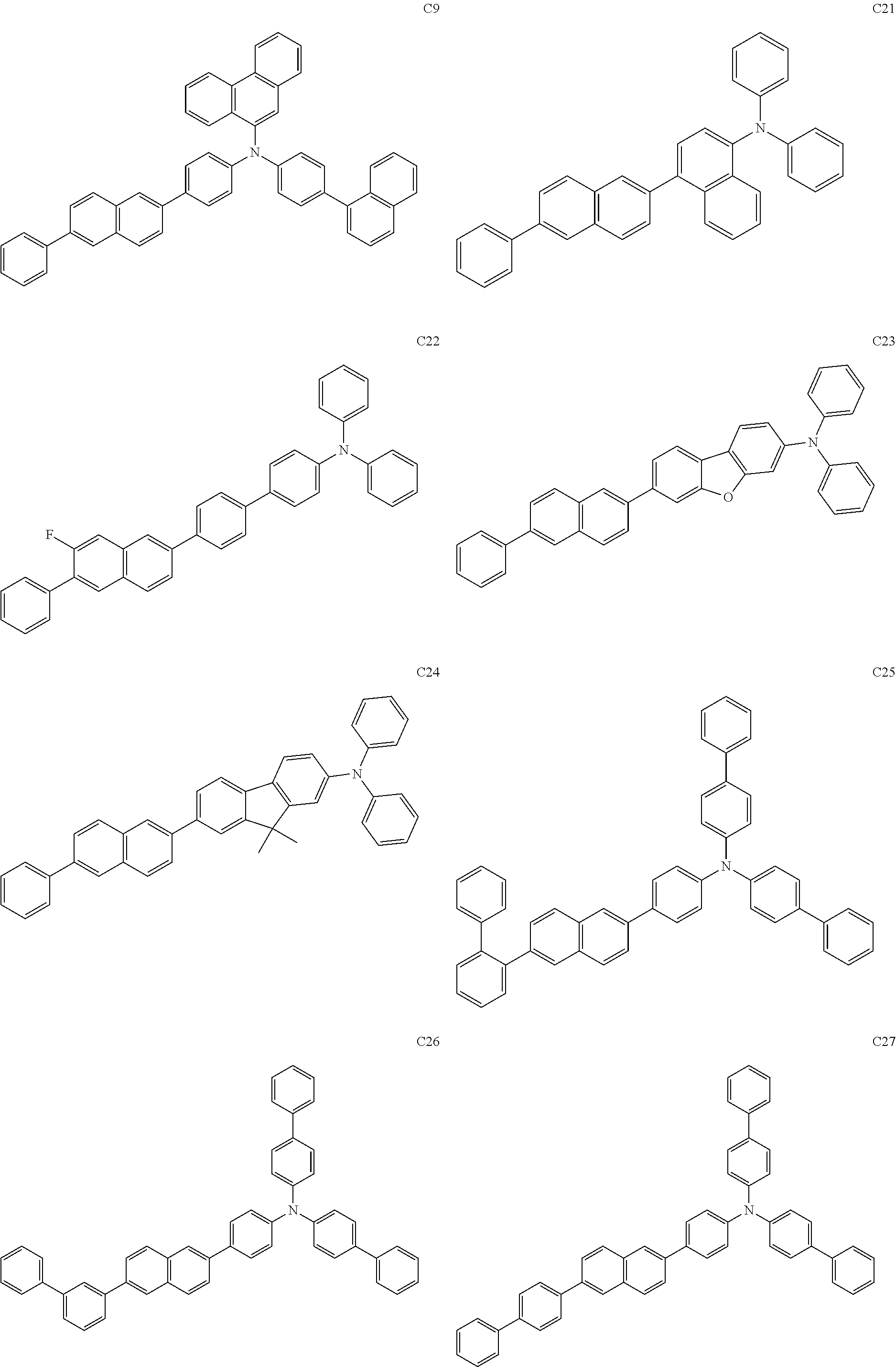

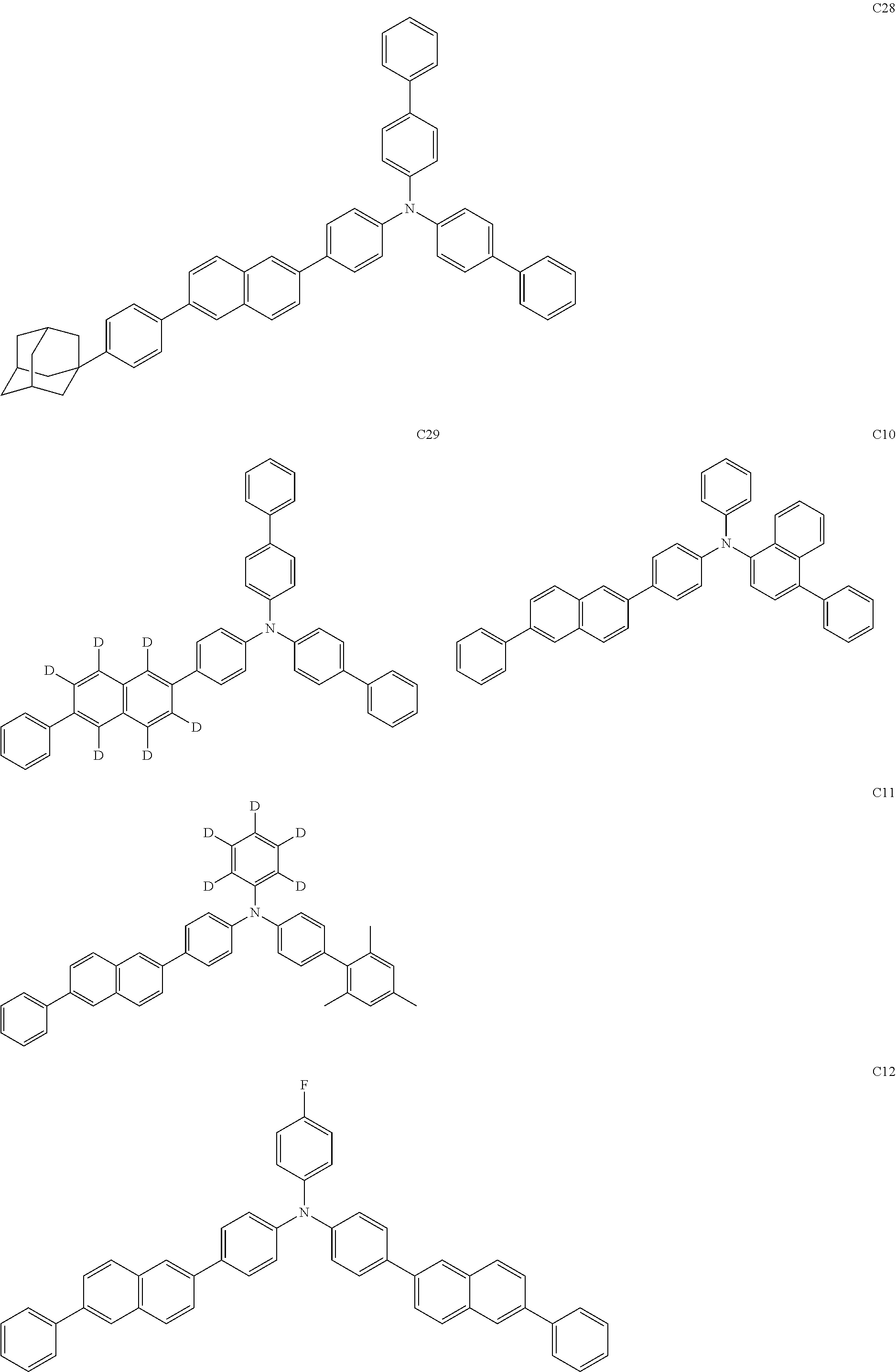 [SMILES: c1ccc(-c2ccc(N(c3ccc(-c4ccccc4)cc3)c3ccc(-c4ccc5cc(-c6ccc(C78CC9CC(CC(C9)C7)C8)cc6)ccc5c4)cc3)cc2)cc1 ; [2H]c1c(-c2ccc(N(c3ccc(-c4ccccc4)cc3)c3ccc(-c4ccccc4)cc3)cc2)c([2H])c2c([2H])c([2H])c(-c3ccccc3)c([2H])c2c1[2H] ; c1ccc(-c2ccc3cc(-c4ccc(N(c5ccccc5)c5ccc(-c6ccccc6)c6ccccc56)cc4)ccc3c2)cc1 ; [2H]c1c([2H])c([2H])c(N(c2ccc(-c3ccc4cc(-c5ccccc5)ccc4c3)cc2)c2ccc(-c3c(C)cc(C)cc3C)cc2)c([2H])c1[2H] ; Fc1ccc(N(c2ccc(-c3ccc4cc(-c5ccccc5)ccc4c3)cc2)c2ccc(-c3ccc4cc(-c5ccccc5)ccc4c3)cc2)cc1]

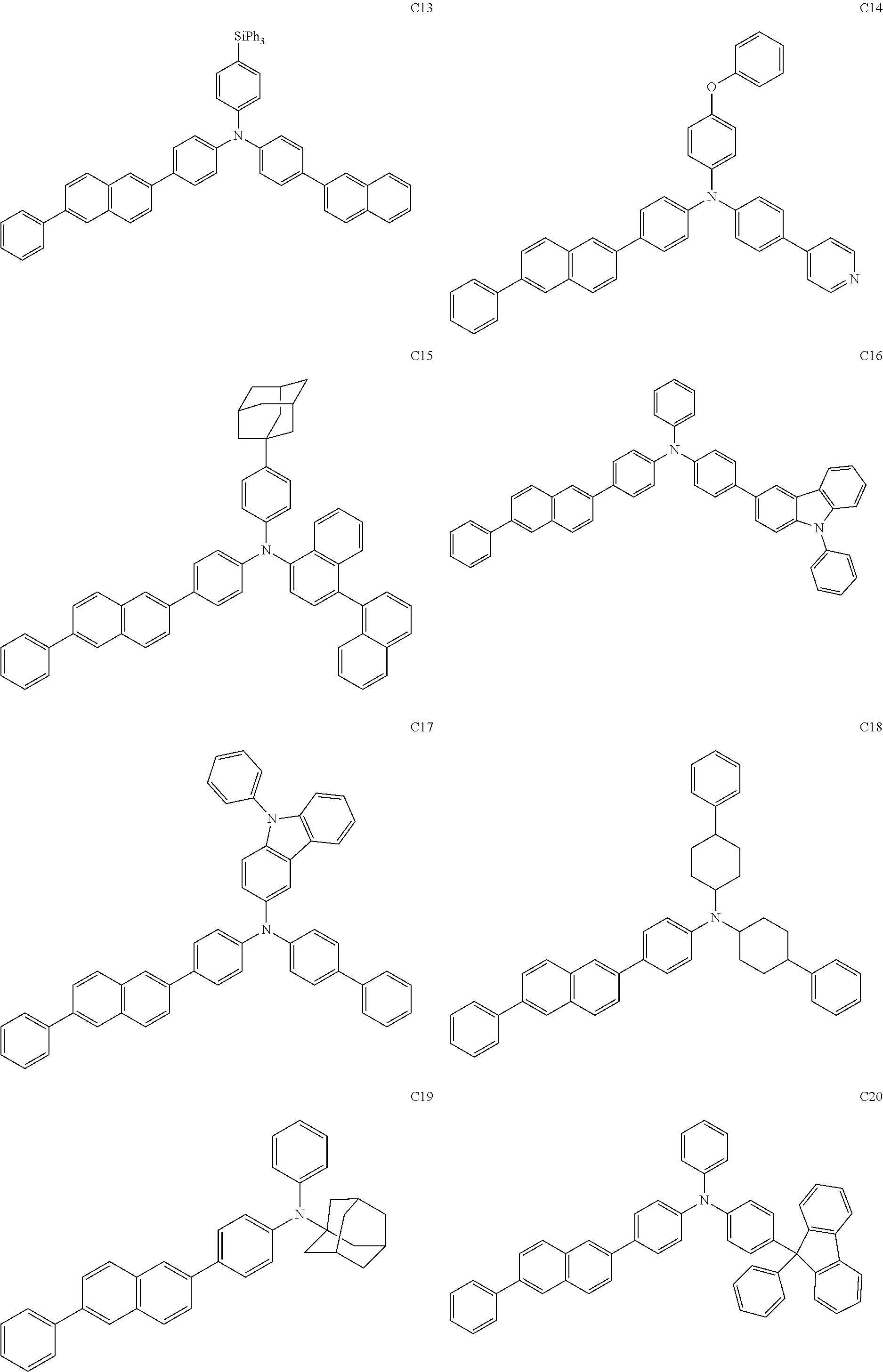

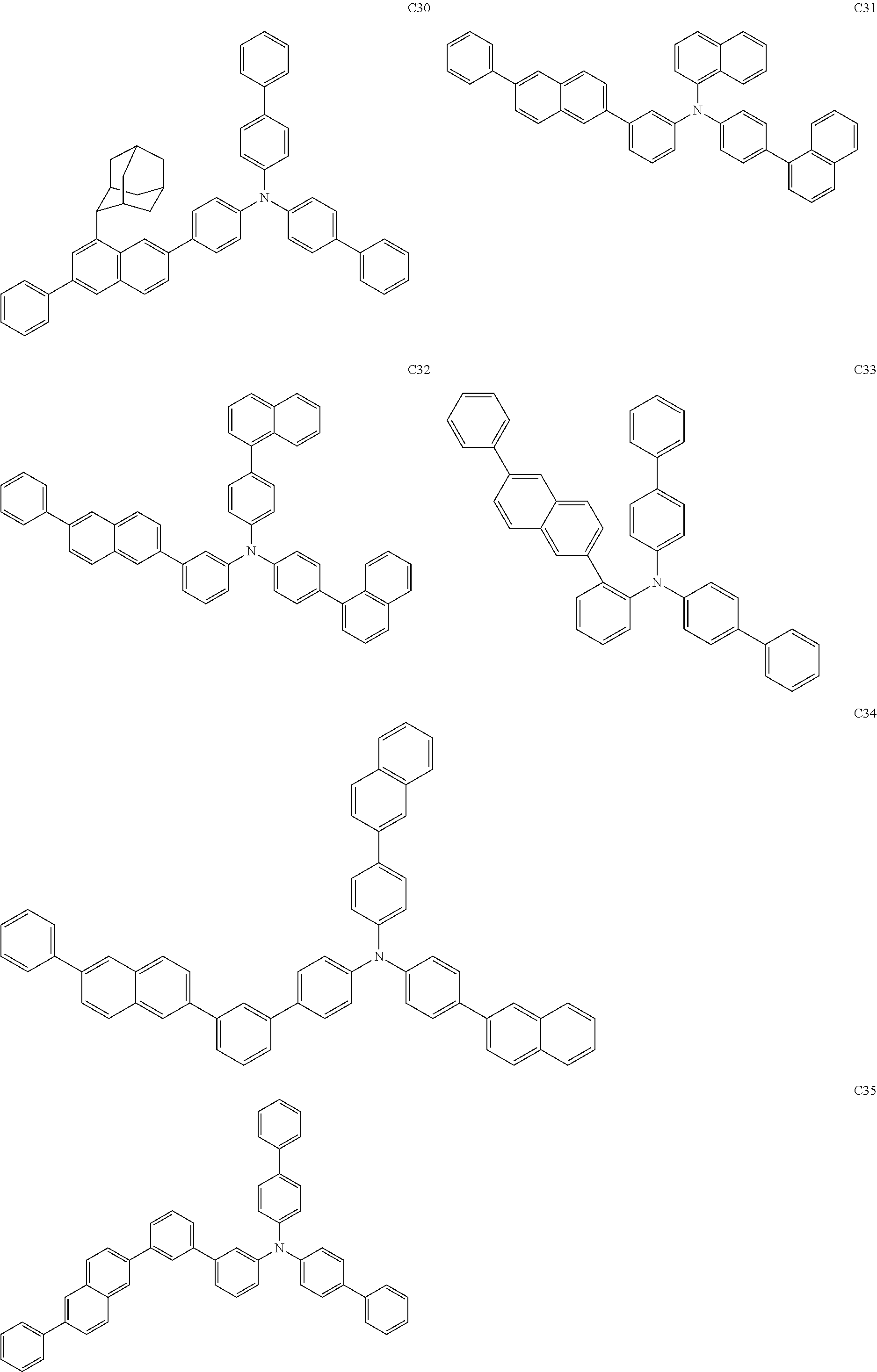

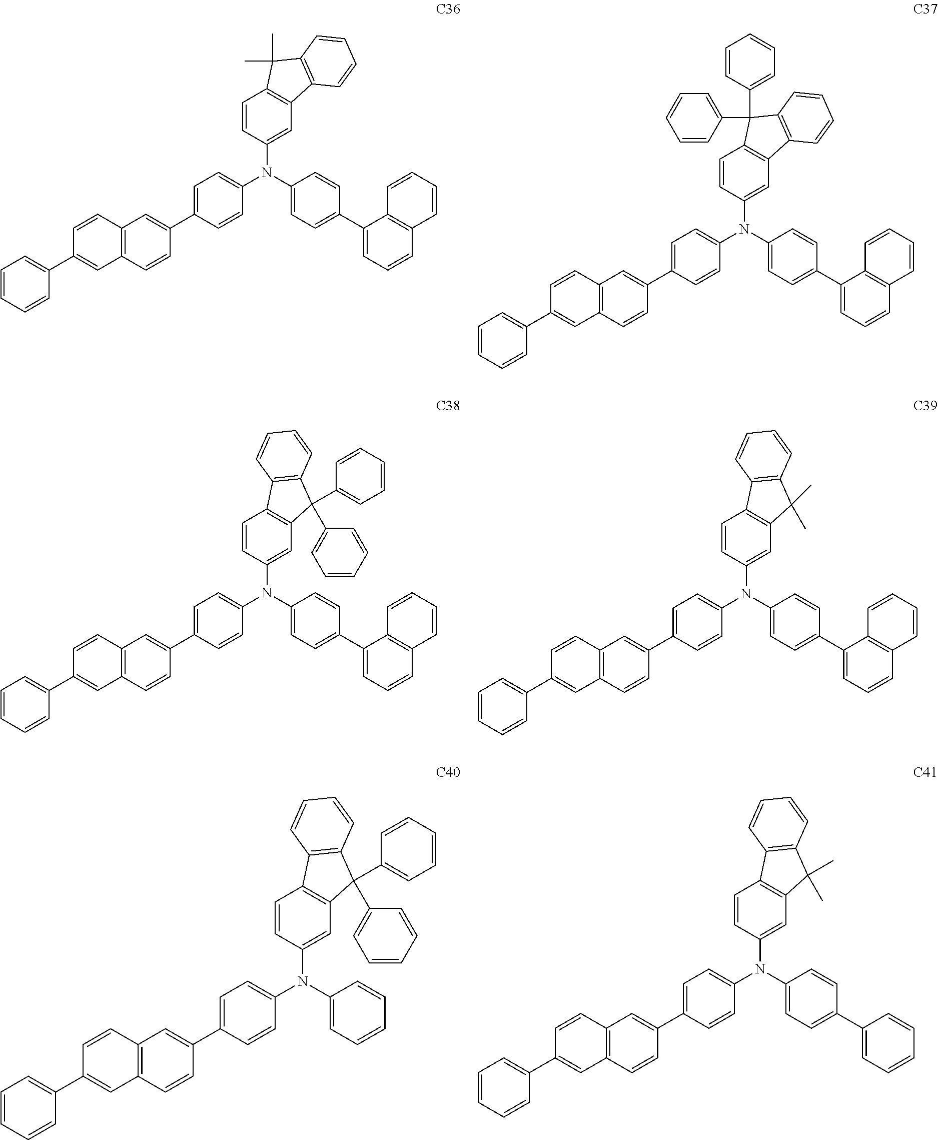

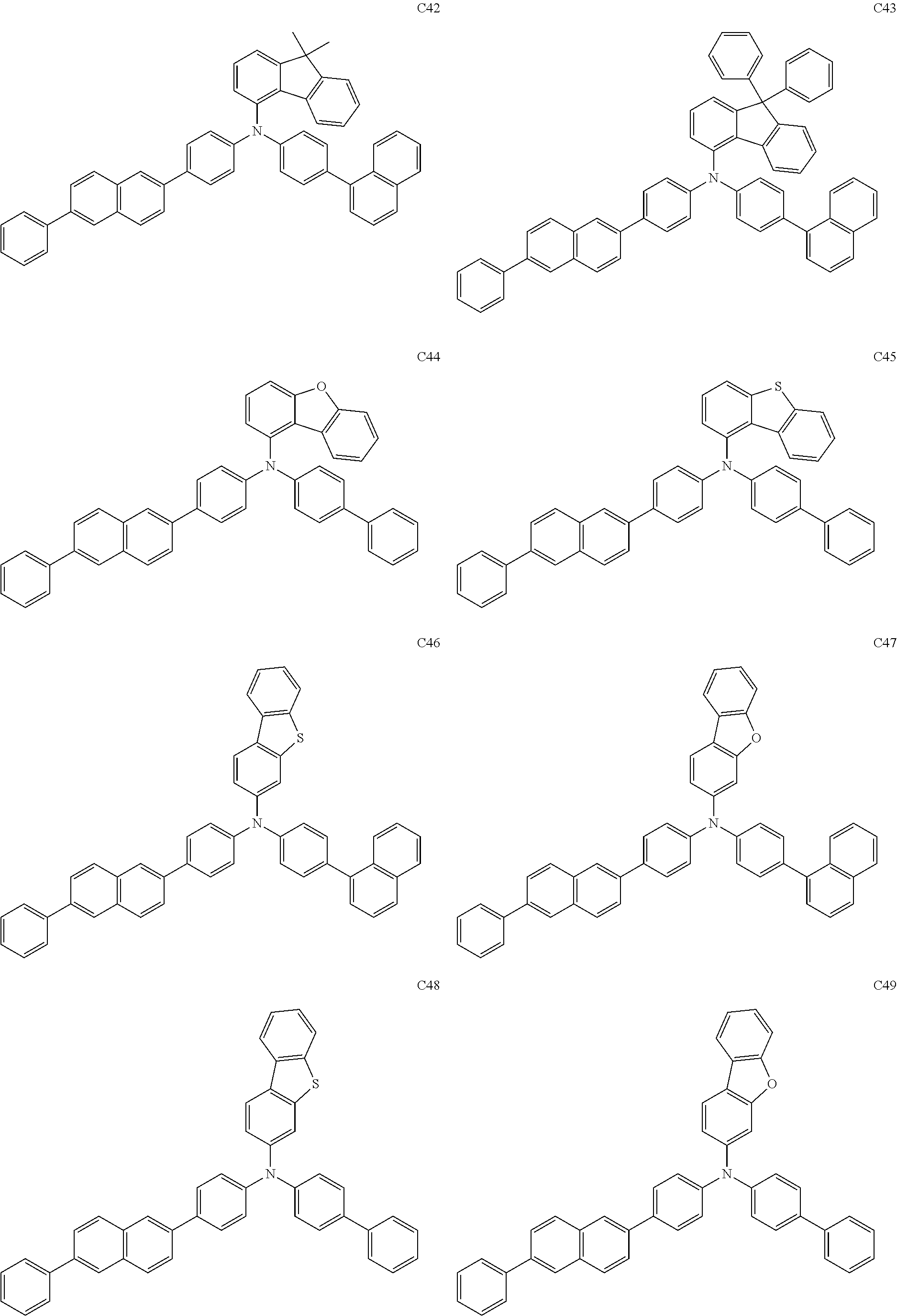

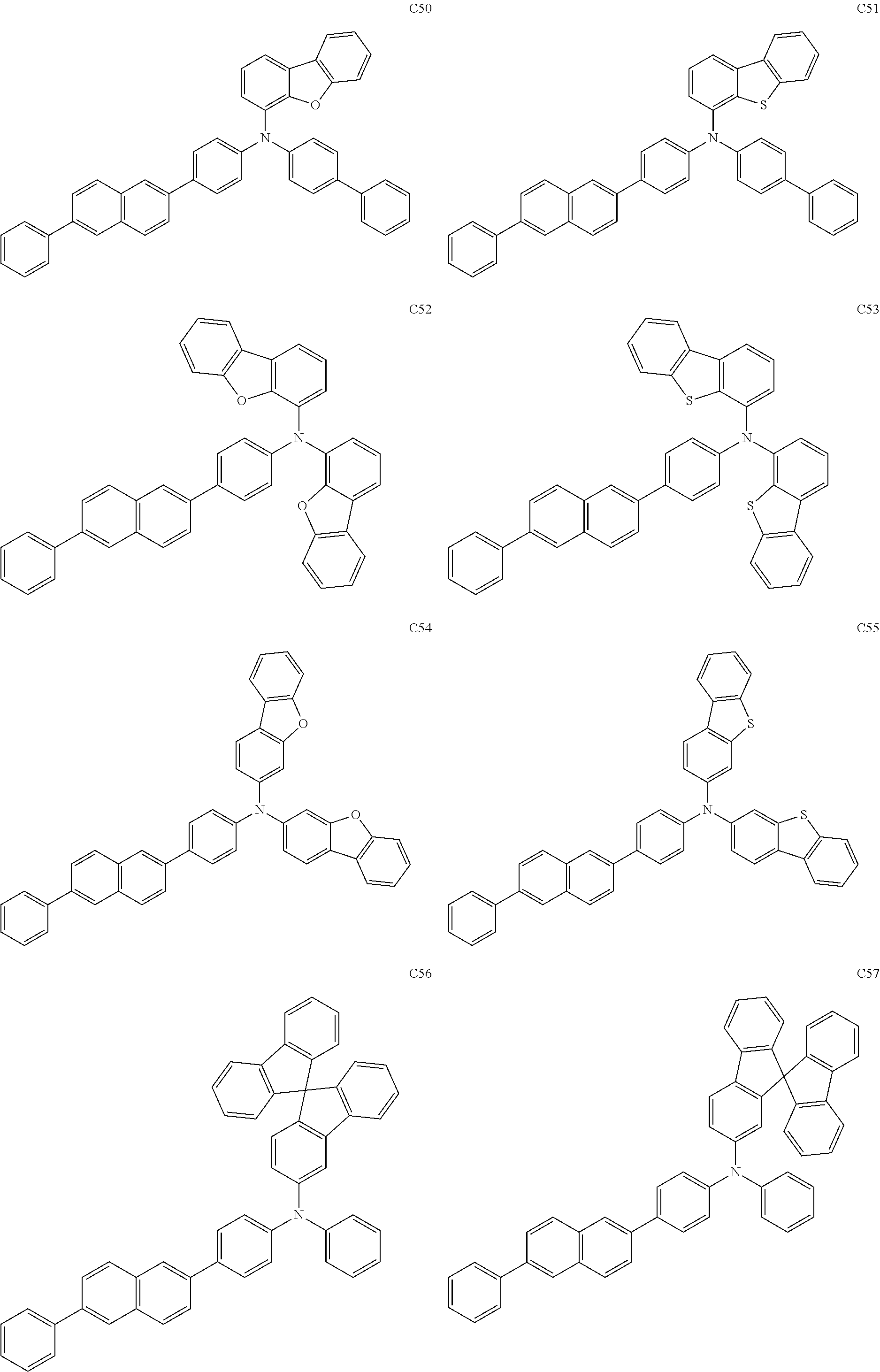

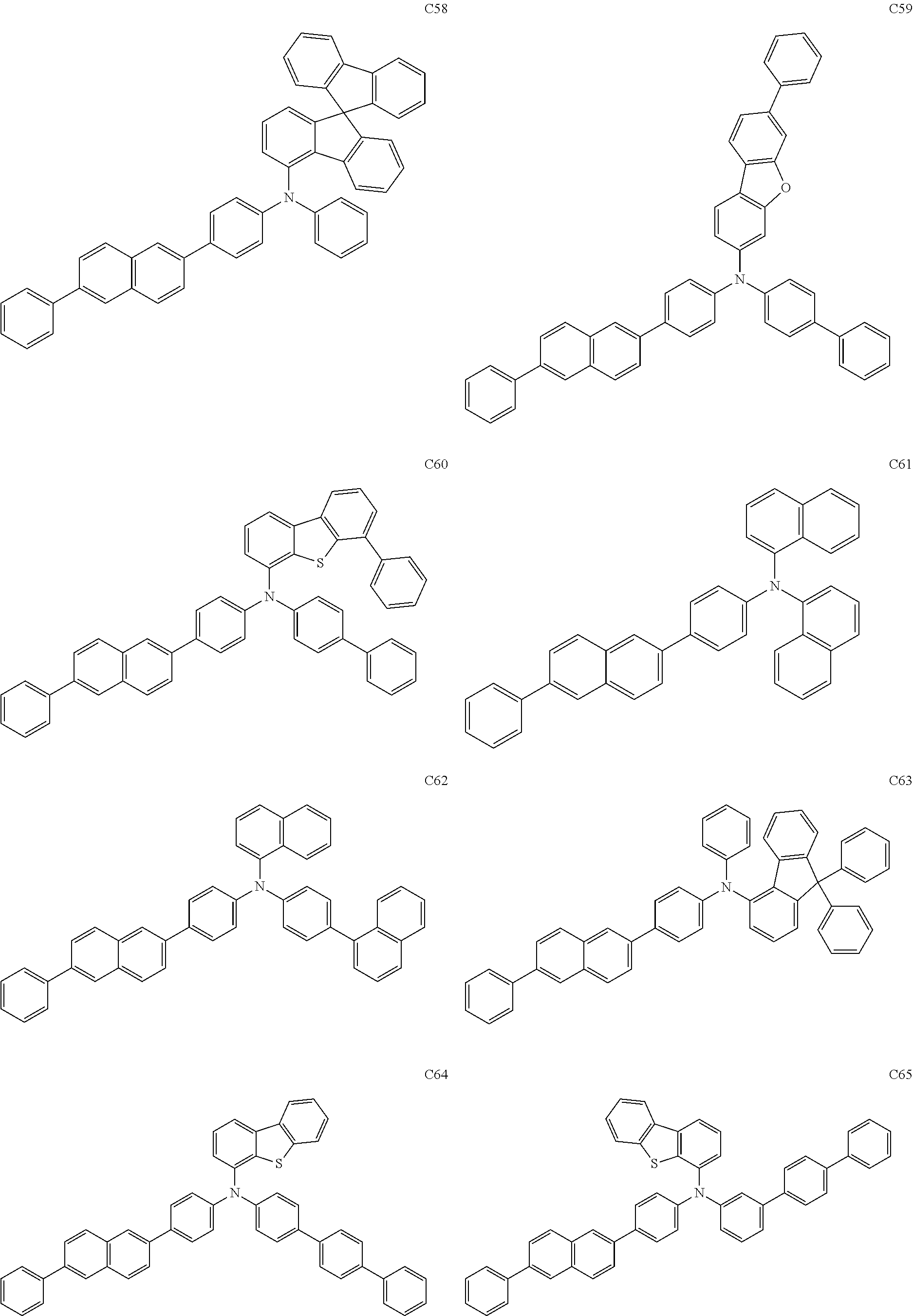

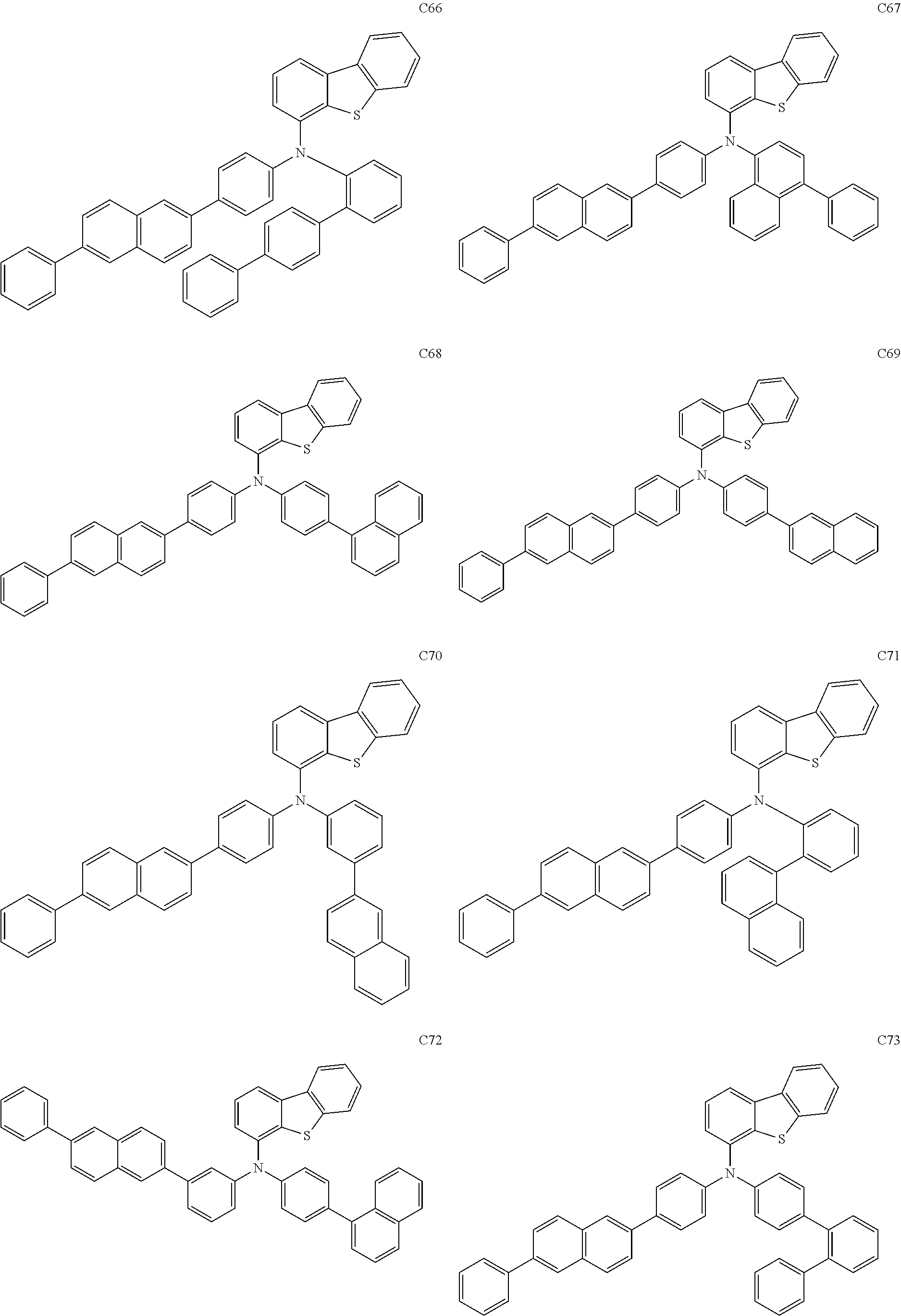

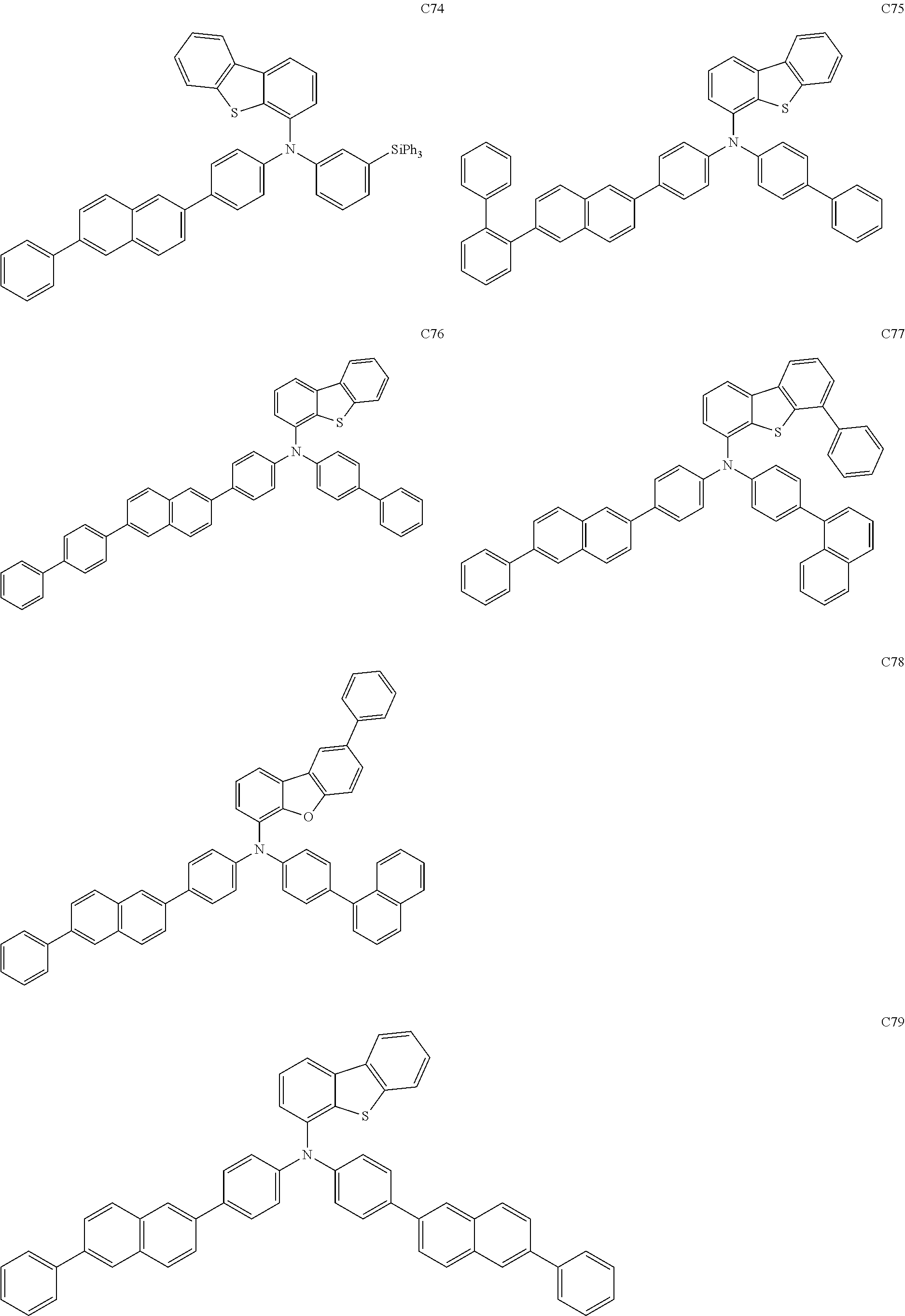

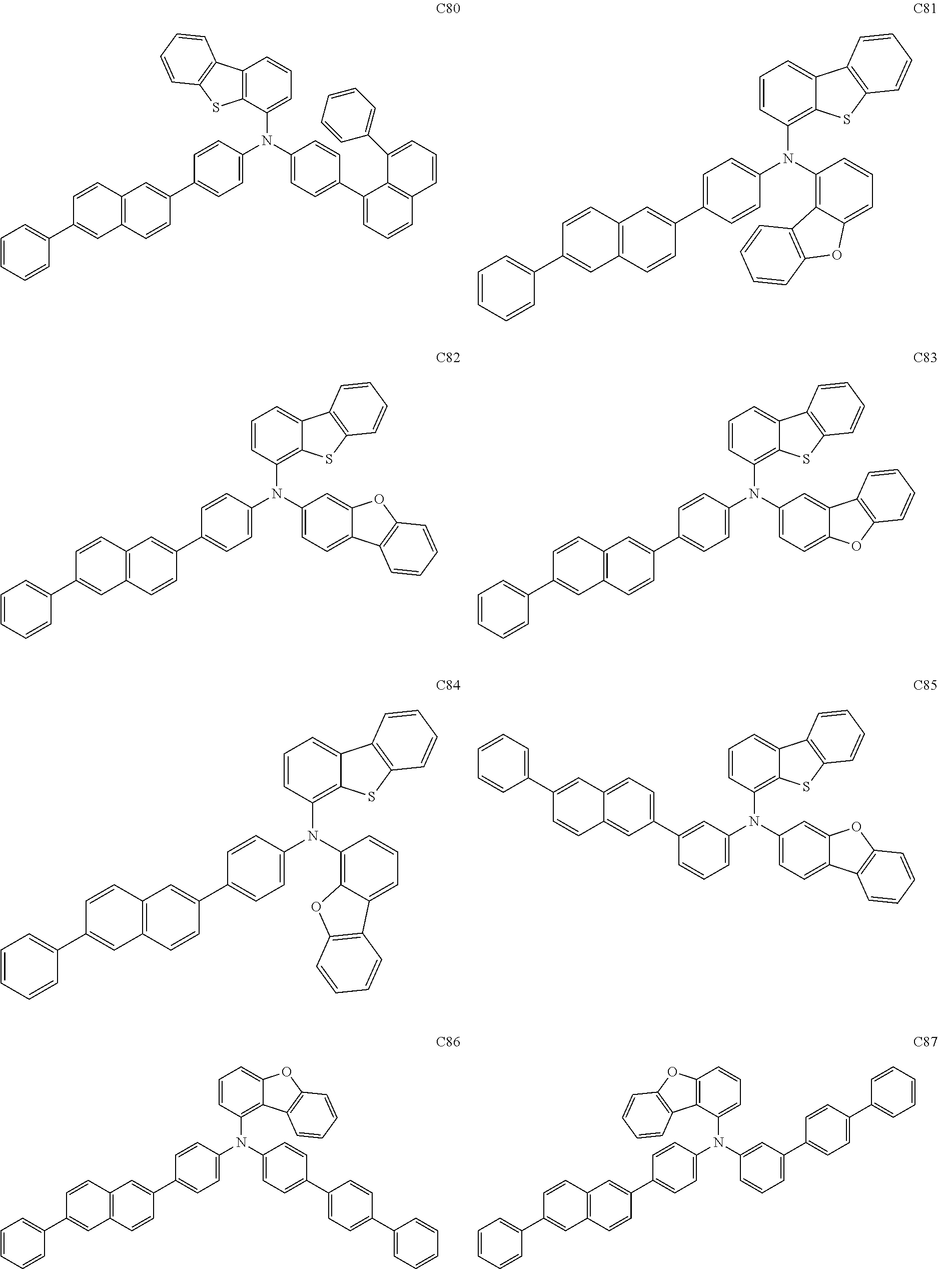

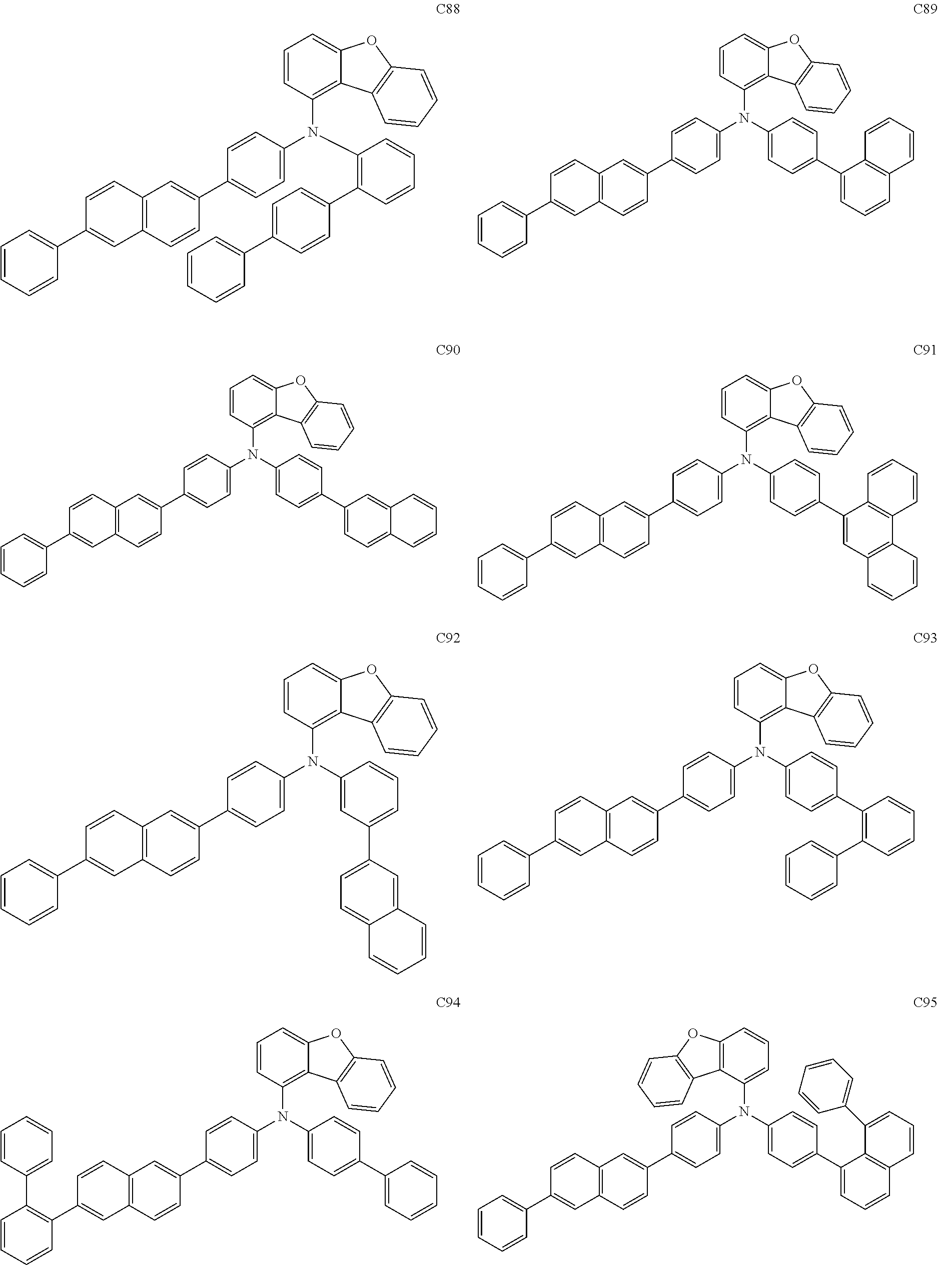

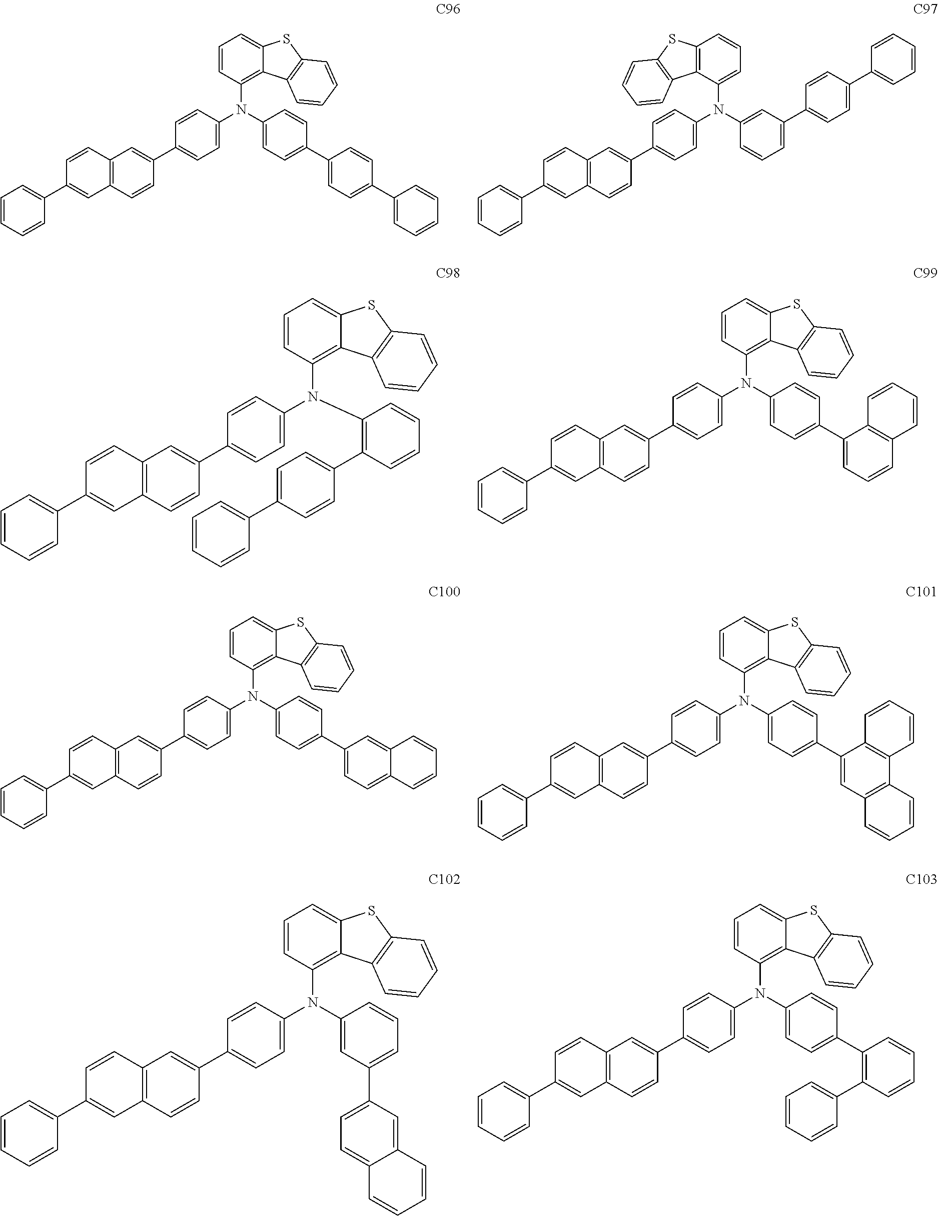

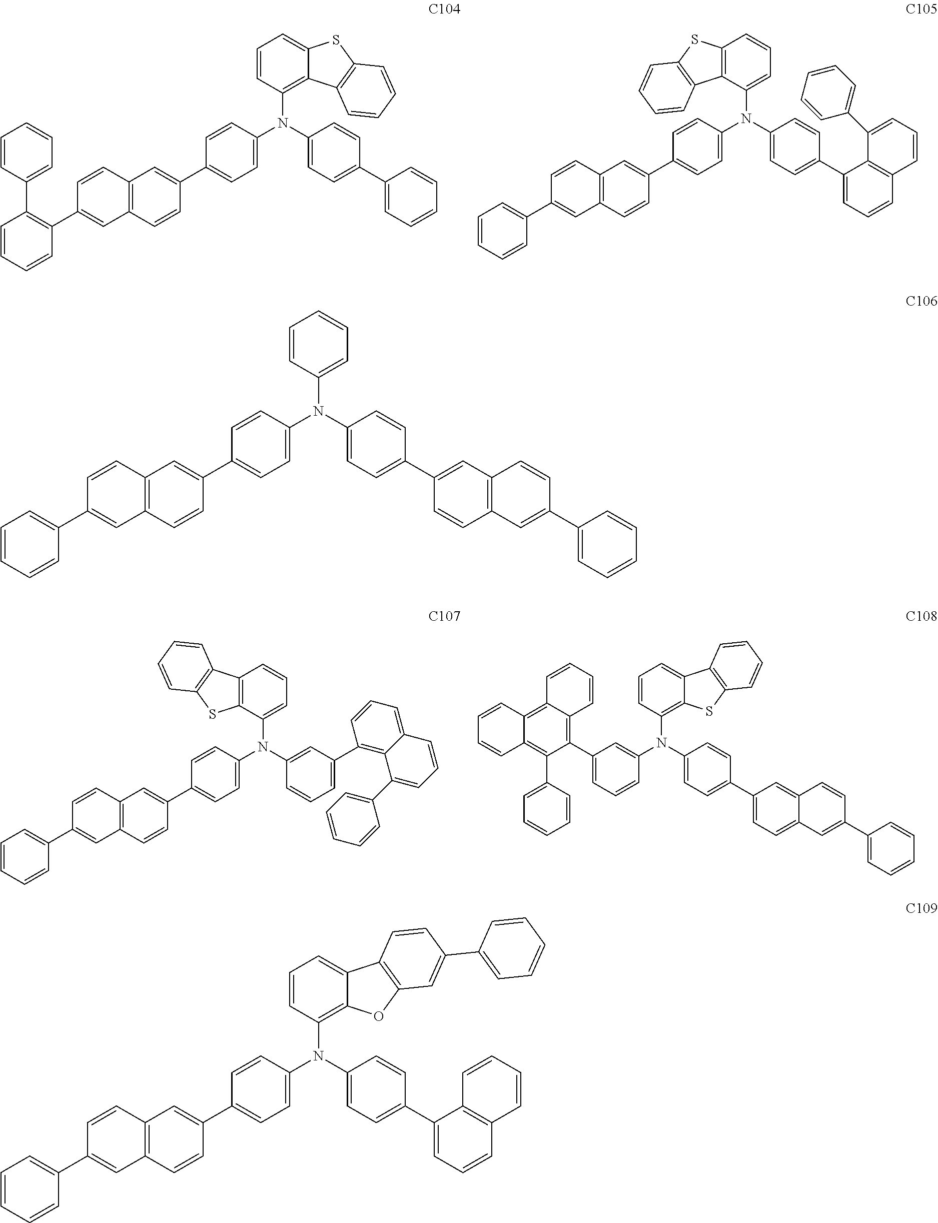

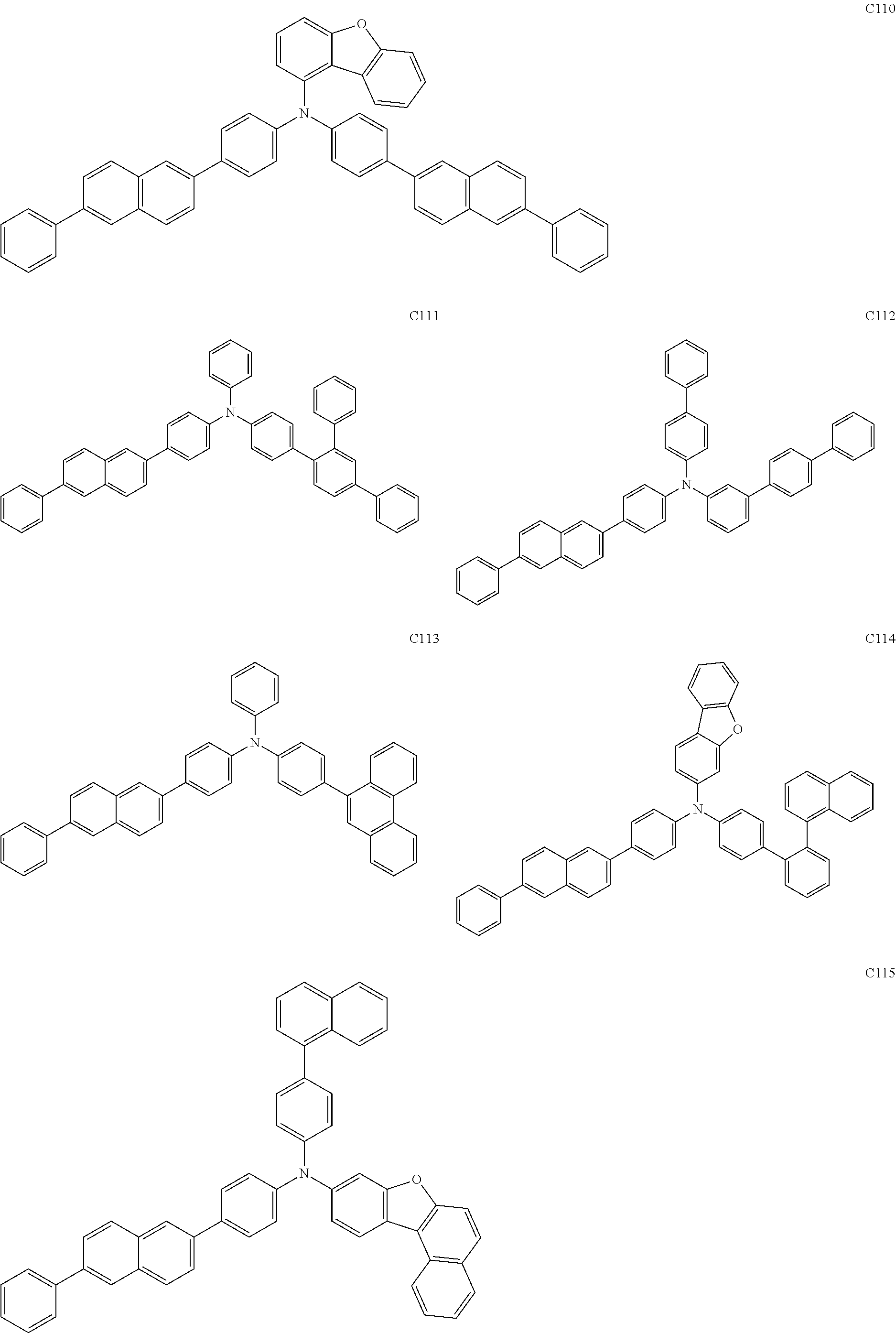

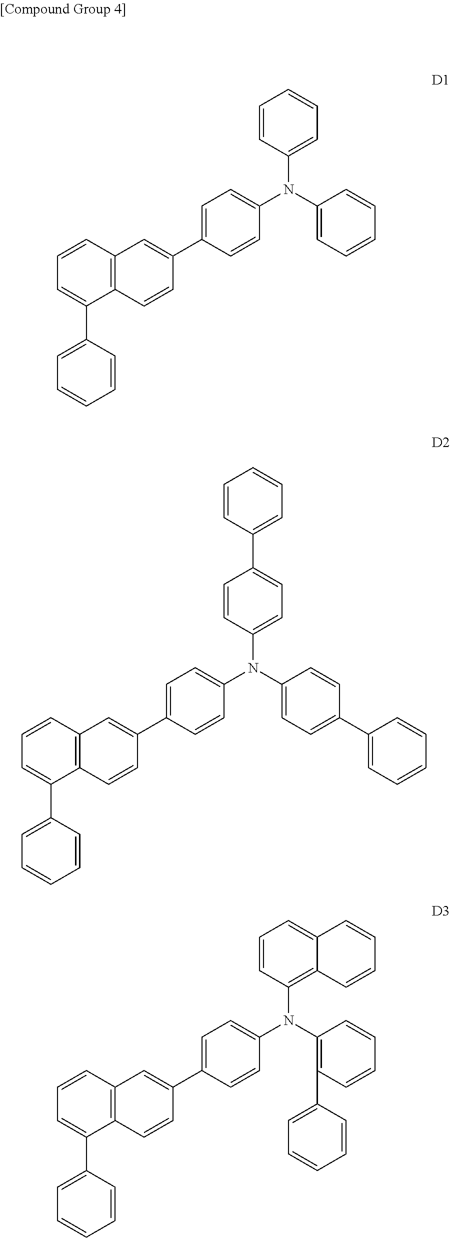 [SMILES: c1ccc(-c2cccc3cc(-c4ccc(N(c5ccccc5)c5ccccc5)cc4)ccc23)cc1 ; c1ccc(-c2ccc(N(c3ccc(-c4ccccc4)cc3)c3ccc(-c4ccc5c(-c6ccccc6)cccc5c4)cc3)cc2)cc1 ; c1ccc(-c2ccccc2N(c2ccc(-c3ccc4c(-c5ccccc5)cccc4c3)cc2)c2cccc3ccccc23)cc1]

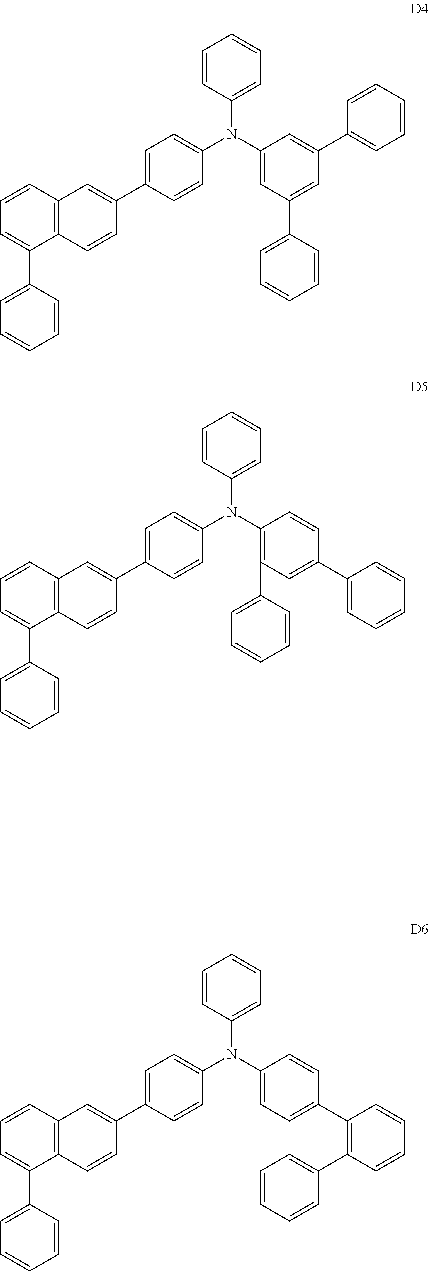 [SMILES: c1ccc(-c2cc(-c3ccccc3)cc(N(c3ccccc3)c3ccc(-c4ccc5c(-c6ccccc6)cccc5c4)cc3)c2)cc1 ; c1ccc(-c2ccc(N(c3ccccc3)c3ccc(-c4ccc5c(-c6ccccc6)cccc5c4)cc3)c(-c3ccccc3)c2)cc1 ; c1ccc(-c2ccccc2-c2ccc(N(c3ccccc3)c3ccc(-c4ccc5c(-c6ccccc6)cccc5c4)cc3)cc2)cc1]

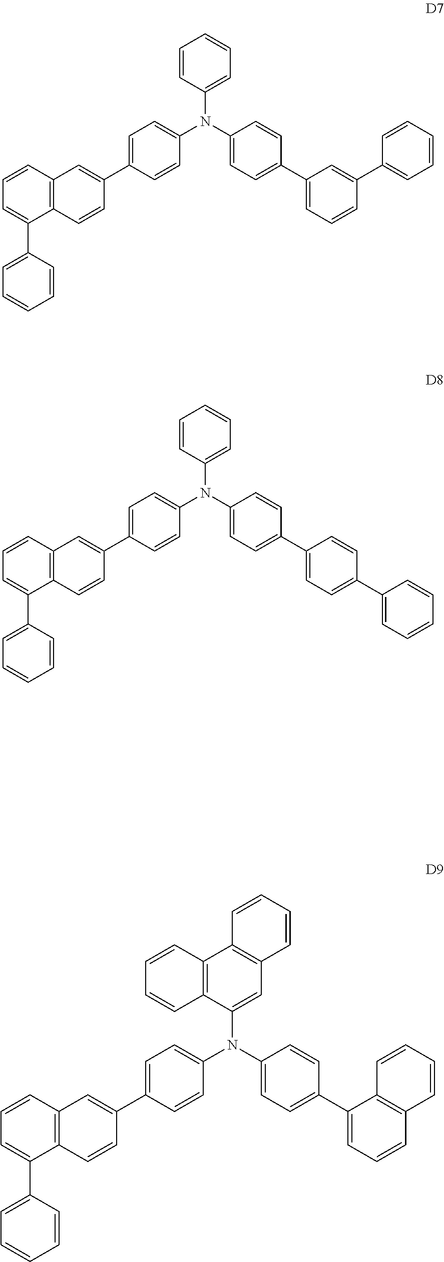 [SMILES: c1ccc(-c2cccc(-c3ccc(N(c4ccccc4)c4ccc(-c5ccc6c(-c7ccccc7)cccc6c5)cc4)cc3)c2)cc1 ; c1ccc(-c2ccc(-c3ccc(N(c4ccccc4)c4ccc(-c5ccc6c(-c7ccccc7)cccc6c5)cc4)cc3)cc2)cc1 ; c1ccc(-c2cccc3cc(-c4ccc(N(c5ccc(-c6cccc7ccccc67)cc5)c5cc6ccccc6c6ccccc56)cc4)ccc23)cc1]

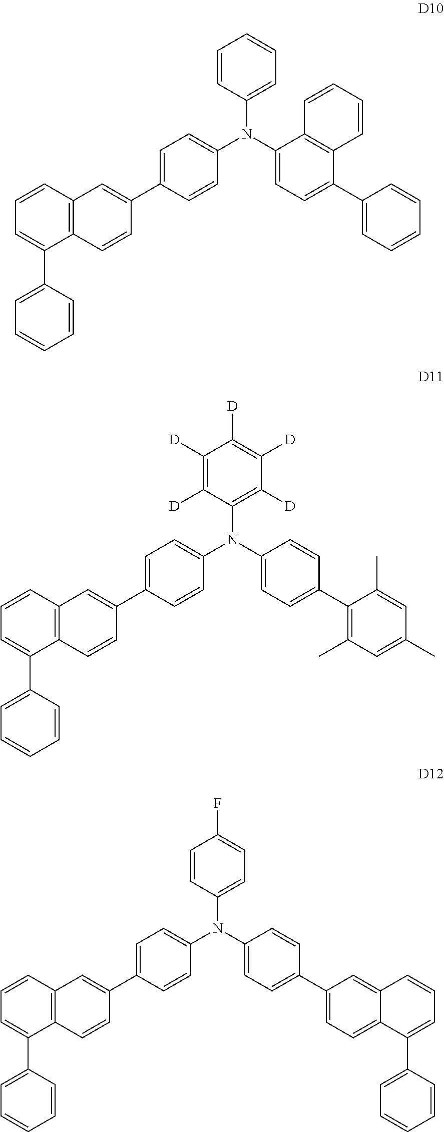 [SMILES: c1ccc(-c2cccc3cc(-c4ccc(N(c5ccccc5)c5ccc(-c6ccccc6)c6ccccc56)cc4)ccc23)cc1 ; [2H]c1c([2H])c([2H])c(N(c2ccc(-c3ccc4c(-c5ccccc5)cccc4c3)cc2)c2ccc(-c3c(C)cc(C)cc3C)cc2)c([2H])c1[2H] ; Fc1ccc(N(c2ccc(-c3ccc4c(-c5ccccc5)cccc4c3)cc2)c2ccc(-c3ccc4c(-c5ccccc5)cccc4c3)cc2)cc1]

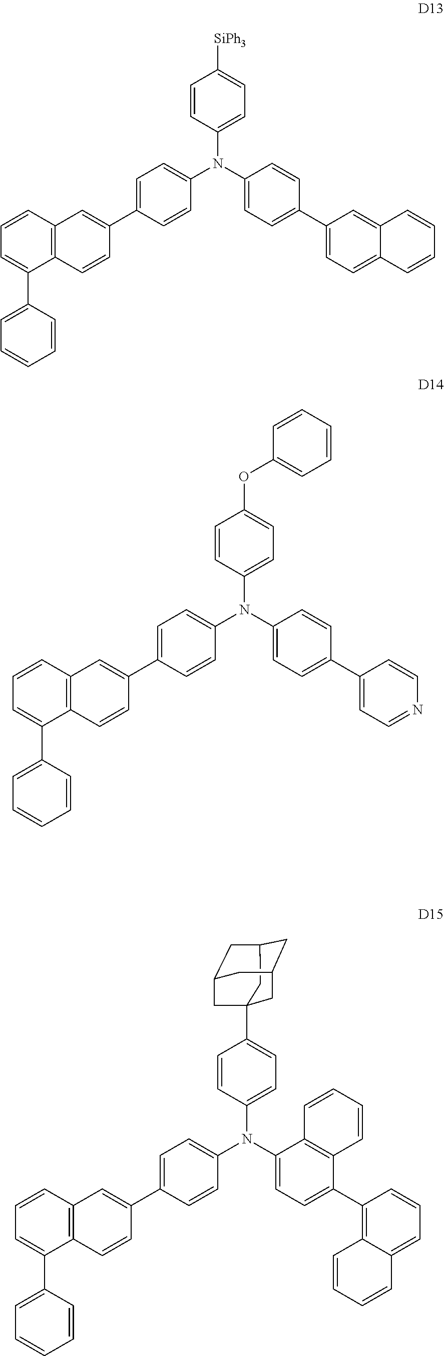

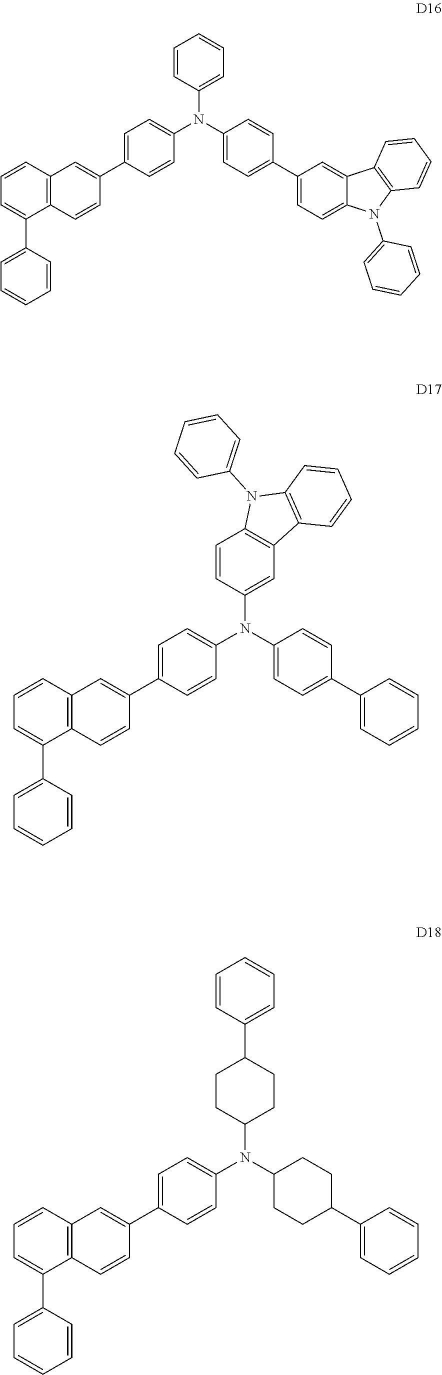 [SMILES: c1ccc(-c2cccc3cc(-c4ccc(N(c5ccccc5)c5ccc(-c6ccc7c(c6)c6ccccc6n7-c6ccccc6)cc5)cc4)ccc23)cc1 ; c1ccc(-c2ccc(N(c3ccc(-c4ccc5c(-c6ccccc6)cccc5c4)cc3)c3ccc4c(c3)c3ccccc3n4-c3ccccc3)cc2)cc1 ; c1ccc(-c2cccc3cc(-c4ccc(N(C5CCC(c6ccccc6)CC5)C5CCC(c6ccccc6)CC5)cc4)ccc23)cc1]

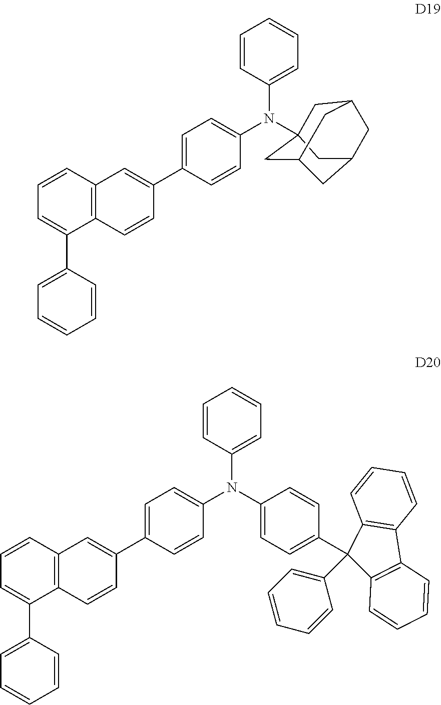 [SMILES: c1ccc(-c2cccc3cc(-c4ccc(N(c5ccccc5)C56CC7CC(CC(C7)C5)C6)cc4)ccc23)cc1 ; c1ccc(-c2cccc3cc(-c4ccc(N(c5ccccc5)c5ccc(C6(c7ccccc7)c7ccccc7-c7ccccc76)cc5)cc4)ccc23)cc1]

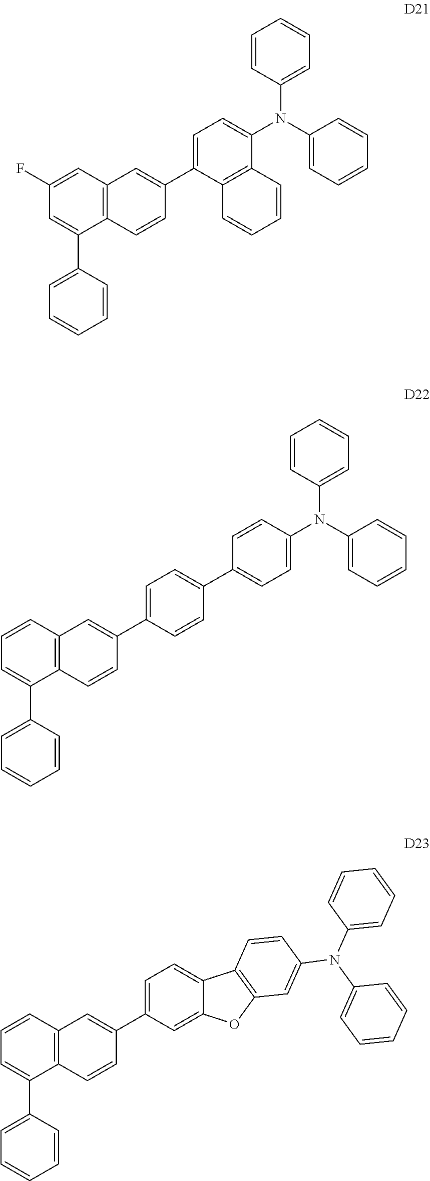 [SMILES: Fc1cc(-c2ccccc2)c2ccc(-c3ccc(N(c4ccccc4)c4ccccc4)c4ccccc34)cc2c1 ; c1ccc(-c2cccc3cc(-c4ccc(-c5ccc(N(c6ccccc6)c6ccccc6)cc5)cc4)ccc23)cc1 ; c1ccc(-c2cccc3cc(-c4ccc5c(c4)oc4cc(N(c6ccccc6)c6ccccc6)ccc45)ccc23)cc1]

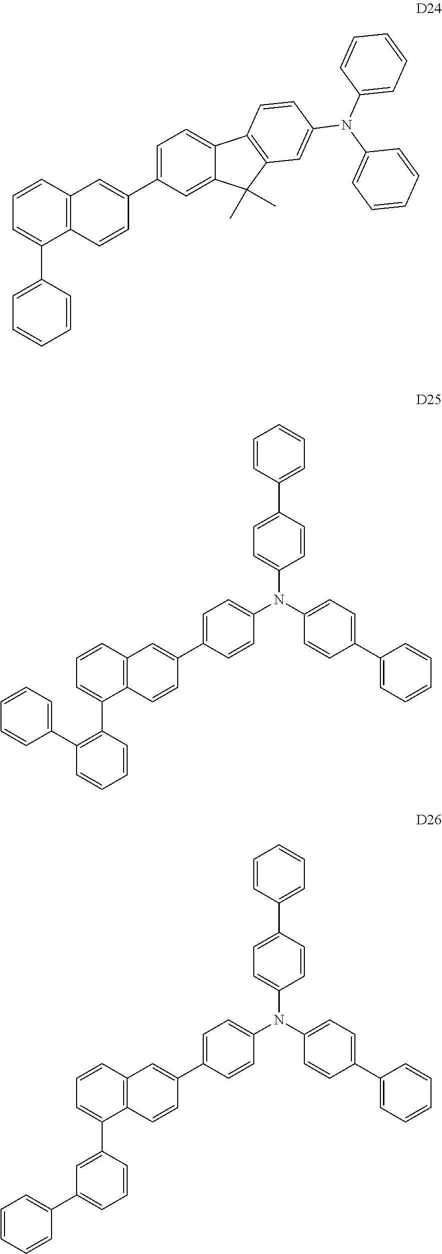 [SMILES: CC1(C)c2cc(-c3ccc4c(-c5ccccc5)cccc4c3)ccc2-c2ccc(N(c3ccccc3)c3ccccc3)cc21 ; c1ccc(-c2ccc(N(c3ccc(-c4ccccc4)cc3)c3ccc(-c4ccc5c(-c6ccccc6-c6ccccc6)cccc5c4)cc3)cc2)cc1 ; c1ccc(-c2ccc(N(c3ccc(-c4ccccc4)cc3)c3ccc(-c4ccc5c(-c6cccc(-c7ccccc7)c6)cccc5c4)cc3)cc2)cc1]

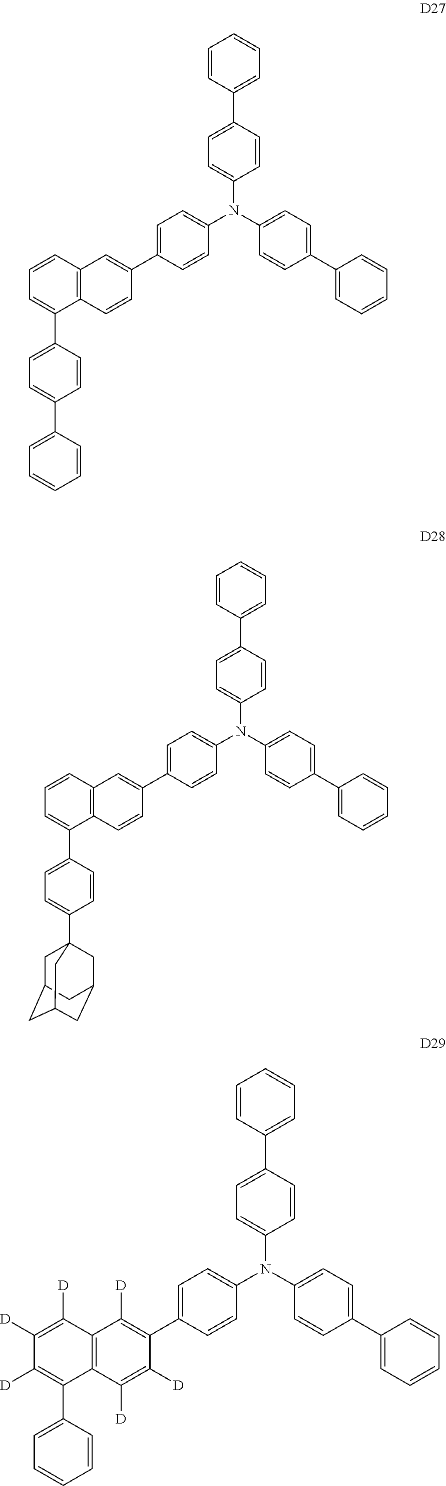 [SMILES: c1ccc(-c2ccc(-c3cccc4cc(-c5ccc(N(c6ccc(-c7ccccc7)cc6)c6ccc(-c7ccccc7)cc6)cc5)ccc34)cc2)cc1 ; c1ccc(-c2ccc(N(c3ccc(-c4ccccc4)cc3)c3ccc(-c4ccc5c(-c6ccc(C78CC9CC(CC(C9)C7)C8)cc6)cccc5c4)cc3)cc2)cc1 ; [2H]c1c([2H])c(-c2ccccc2)c2c([2H])c([2H])c(-c3ccc(N(c4ccc(-c5ccccc5)cc4)c4ccc(-c5ccccc5)cc4)cc3)c([2H])c2c1[2H]]

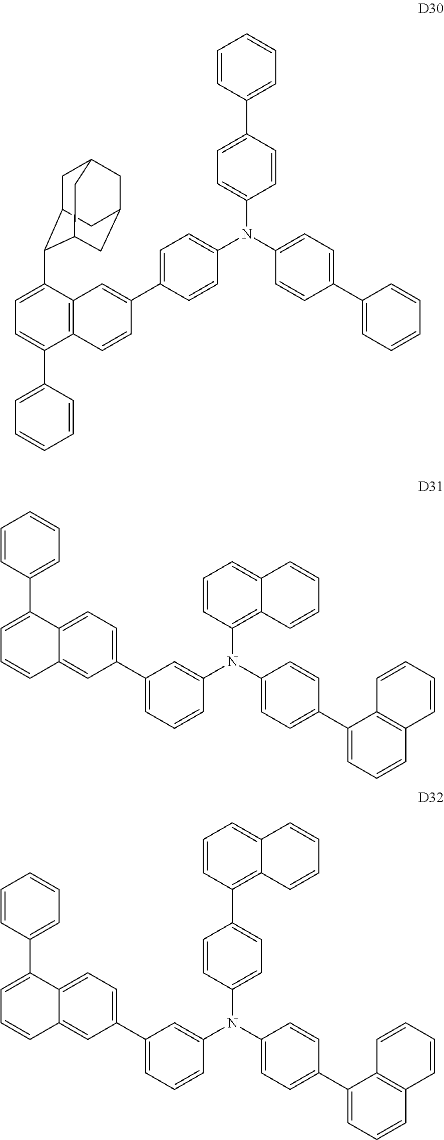 [SMILES: c1ccc(-c2ccc(N(c3ccc(-c4ccccc4)cc3)c3ccc(-c4ccc5c(-c6ccccc6)ccc(C6C7CC8CC(C7)CC6C8)c5c4)cc3)cc2)cc1 ; c1ccc(-c2cccc3cc(-c4cccc(N(c5ccc(-c6cccc7ccccc67)cc5)c5cccc6ccccc56)c4)ccc23)cc1 ; c1ccc(-c2cccc3cc(-c4cccc(N(c5ccc(-c6cccc7ccccc67)cc5)c5ccc(-c6cccc7ccccc67)cc5)c4)ccc23)cc1]

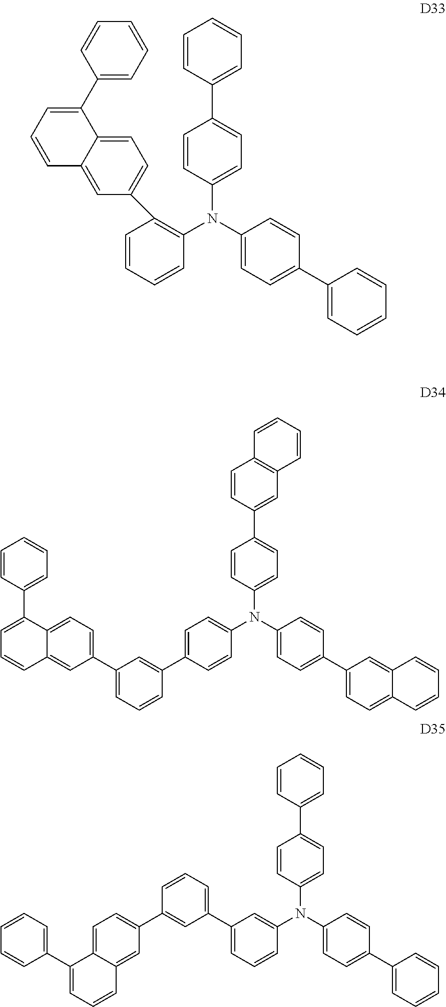 [SMILES: c1ccc(-c2ccc(N(c3ccc(-c4ccccc4)cc3)c3ccccc3-c3ccc4c(-c5ccccc5)cccc4c3)cc2)cc1 ; c1ccc(-c2cccc3cc(-c4cccc(-c5ccc(N(c6ccc(-c7ccc8ccccc8c7)cc6)c6ccc(-c7ccc8ccccc8c7)cc6)cc5)c4)ccc23)cc1 ; c1ccc(-c2ccc(N(c3ccc(-c4ccccc4)cc3)c3cccc(-c4cccc(-c5ccc6c(-c7ccccc7)cccc6c5)c4)c3)cc2)cc1]

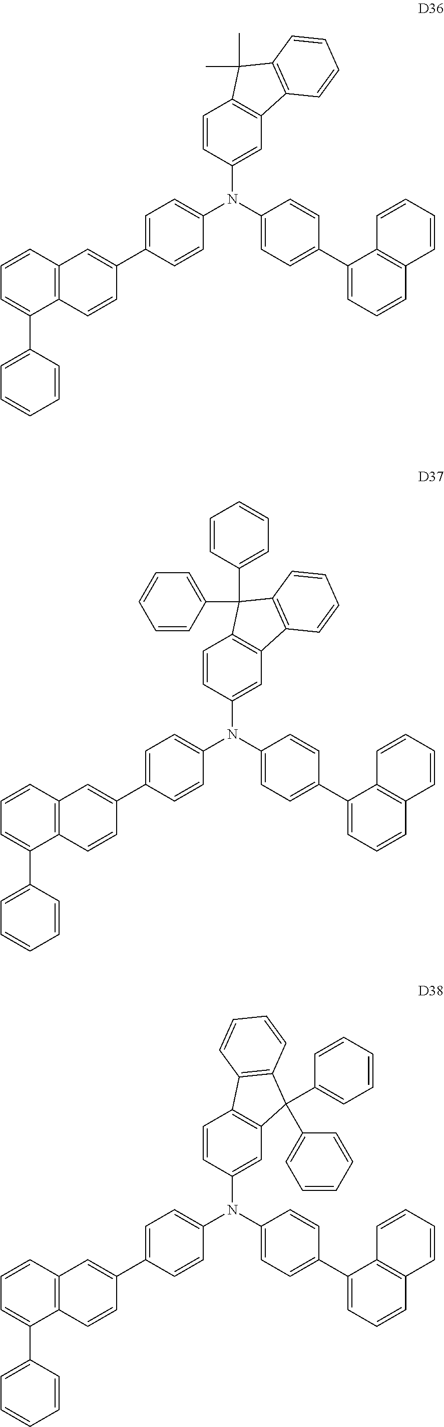 [SMILES: CC1(C)c2ccccc2-c2cc(N(c3ccc(-c4ccc5c(-c6ccccc6)cccc5c4)cc3)c3ccc(-c4cccc5ccccc45)cc3)ccc21 ; c1ccc(-c2cccc3cc(-c4ccc(N(c5ccc(-c6cccc7ccccc67)cc5)c5ccc6c(c5)-c5ccccc5C6(c5ccccc5)c5ccccc5)cc4)ccc23)cc1 ; c1ccc(-c2cccc3cc(-c4ccc(N(c5ccc(-c6cccc7ccccc67)cc5)c5ccc6c(c5)C(c5ccccc5)(c5ccccc5)c5ccccc5-6)cc4)ccc23)cc1]

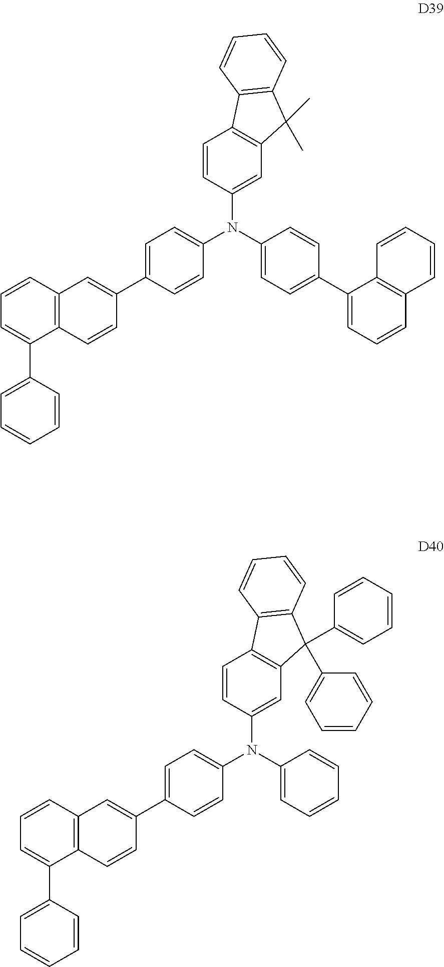 [SMILES: CC1(C)c2ccccc2-c2ccc(N(c3ccc(-c4ccc5c(-c6ccccc6)cccc5c4)cc3)c3ccc(-c4cccc5ccccc45)cc3)cc21 ; c1ccc(-c2cccc3cc(-c4ccc(N(c5ccccc5)c5ccc6c(c5)C(c5ccccc5)(c5ccccc5)c5ccccc5-6)cc4)ccc23)cc1]

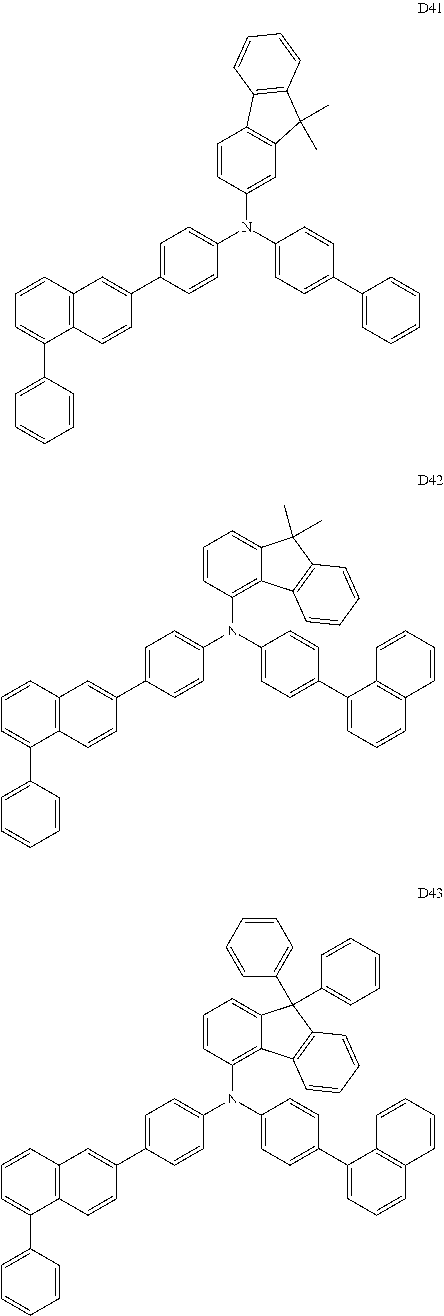 [SMILES: CC1(C)c2ccccc2-c2ccc(N(c3ccc(-c4ccccc4)cc3)c3ccc(-c4ccc5c(-c6ccccc6)cccc5c4)cc3)cc21 ; CC1(C)c2ccccc2-c2c(N(c3ccc(-c4ccc5c(-c6ccccc6)cccc5c4)cc3)c3ccc(-c4cccc5ccccc45)cc3)cccc21 ; c1ccc(-c2cccc3cc(-c4ccc(N(c5ccc(-c6cccc7ccccc67)cc5)c5cccc6c5-c5ccccc5C6(c5ccccc5)c5ccccc5)cc4)ccc23)cc1]

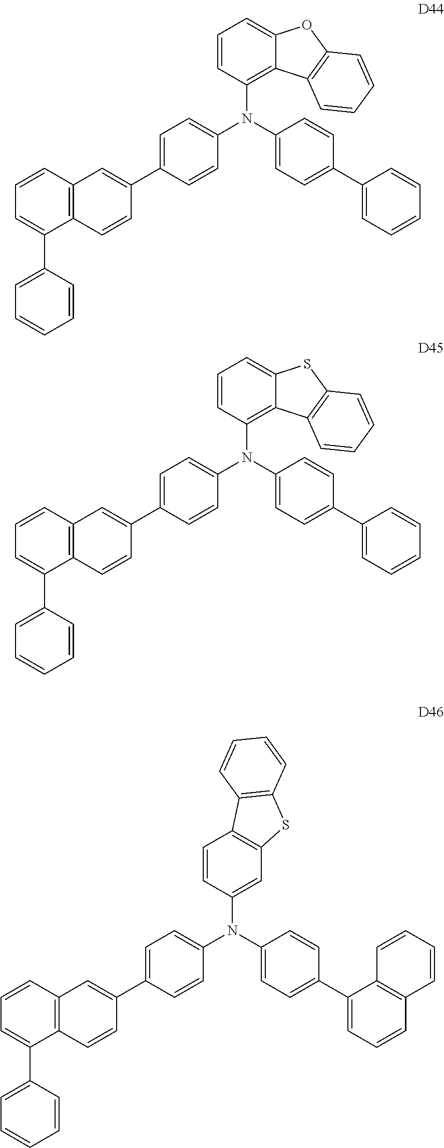 [SMILES: c1ccc(-c2ccc(N(c3ccc(-c4ccc5c(-c6ccccc6)cccc5c4)cc3)c3cccc4oc5ccccc5c34)cc2)cc1 ; c1ccc(-c2ccc(N(c3ccc(-c4ccc5c(-c6ccccc6)cccc5c4)cc3)c3cccc4sc5ccccc5c34)cc2)cc1 ; c1ccc(-c2cccc3cc(-c4ccc(N(c5ccc(-c6cccc7ccccc67)cc5)c5ccc6c(c5)sc5ccccc56)cc4)ccc23)cc1]

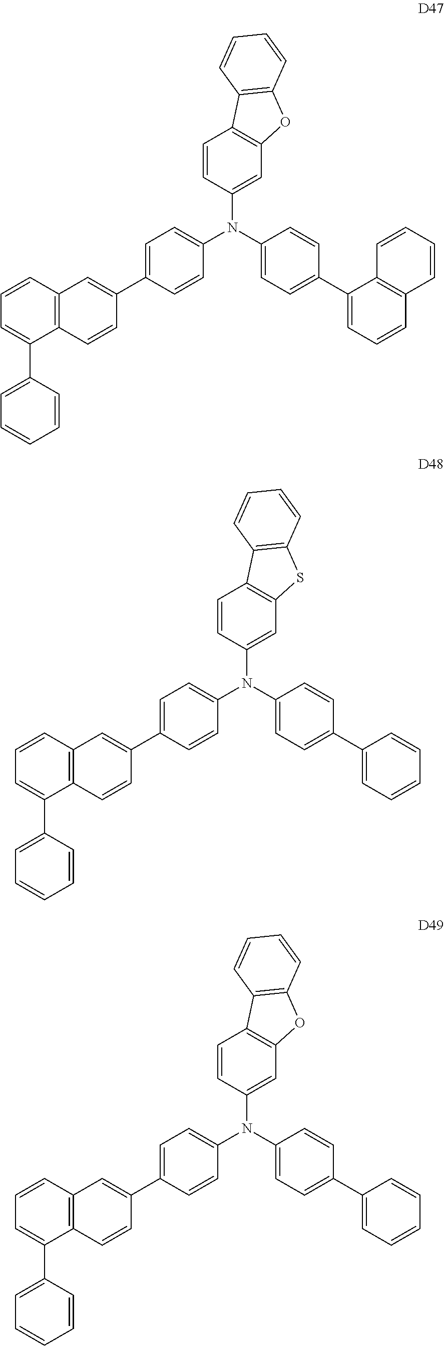 [SMILES: c1ccc(-c2cccc3cc(-c4ccc(N(c5ccc(-c6cccc7ccccc67)cc5)c5ccc6c(c5)oc5ccccc56)cc4)ccc23)cc1 ; c1ccc(-c2ccc(N(c3ccc(-c4ccc5c(-c6ccccc6)cccc5c4)cc3)c3ccc4c(c3)sc3ccccc34)cc2)cc1 ; c1ccc(-c2ccc(N(c3ccc(-c4ccc5c(-c6ccccc6)cccc5c4)cc3)c3ccc4c(c3)oc3ccccc34)cc2)cc1]

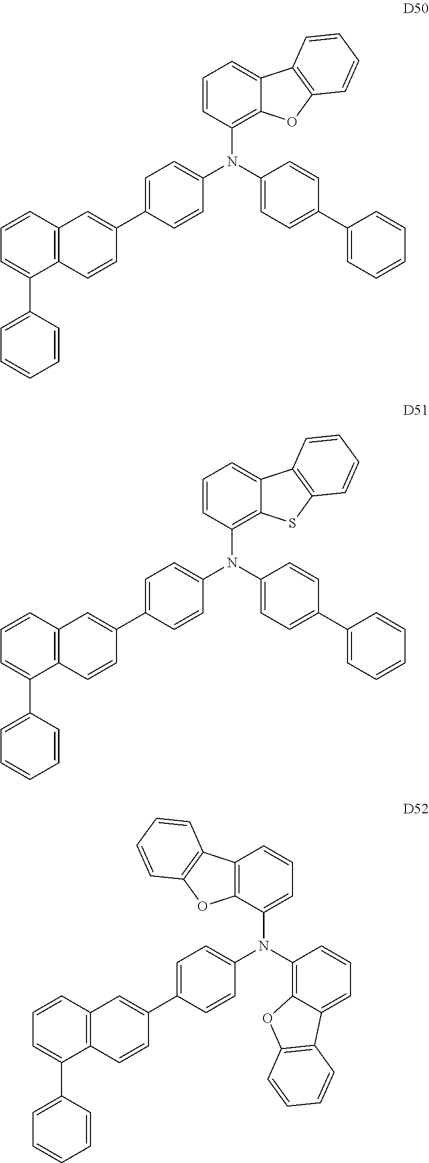 [SMILES: c1ccc(-c2ccc(N(c3ccc(-c4ccc5c(-c6ccccc6)cccc5c4)cc3)c3cccc4c3oc3ccccc34)cc2)cc1 ; c1ccc(-c2ccc(N(c3ccc(-c4ccc5c(-c6ccccc6)cccc5c4)cc3)c3cccc4c3sc3ccccc34)cc2)cc1 ; c1ccc(-c2cccc3cc(-c4ccc(N(c5cccc6c5oc5ccccc56)c5cccc6c5oc5ccccc56)cc4)ccc23)cc1]

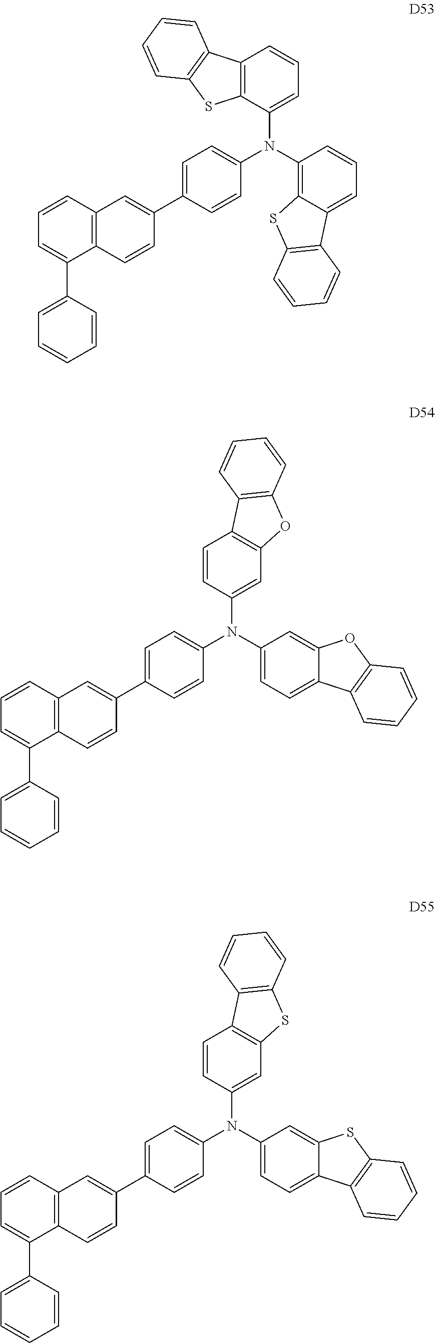 [SMILES: c1ccc(-c2cccc3cc(-c4ccc(N(c5cccc6c5sc5ccccc56)c5cccc6c5sc5ccccc56)cc4)ccc23)cc1 ; c1ccc(-c2cccc3cc(-c4ccc(N(c5ccc6c(c5)oc5ccccc56)c5ccc6c(c5)oc5ccccc56)cc4)ccc23)cc1 ; c1ccc(-c2cccc3cc(-c4ccc(N(c5ccc6c(c5)sc5ccccc56)c5ccc6c(c5)sc5ccccc56)cc4)ccc23)cc1]

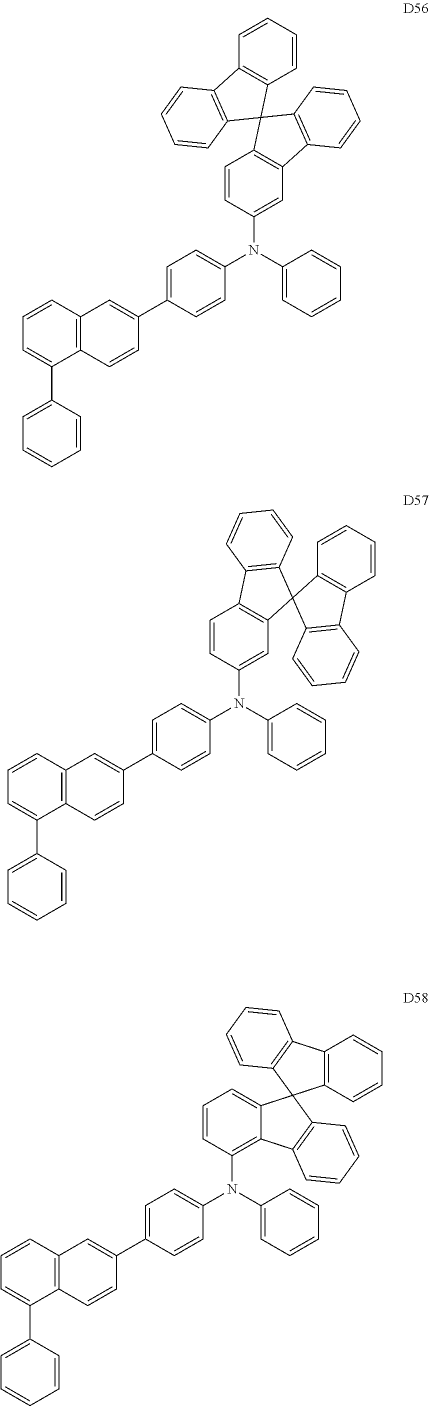 [SMILES: c1ccc(-c2cccc3cc(-c4ccc(N(c5ccccc5)c5ccc6c(c5)-c5ccccc5C65c6ccccc6-c6ccccc65)cc4)ccc23)cc1 ; c1ccc(-c2cccc3cc(-c4ccc(N(c5ccccc5)c5ccc6c(c5)C5(c7ccccc7-c7ccccc75)c5ccccc5-6)cc4)ccc23)cc1 ; c1ccc(-c2cccc3cc(-c4ccc(N(c5ccccc5)c5cccc6c5-c5ccccc5C65c6ccccc6-c6ccccc65)cc4)ccc23)cc1]

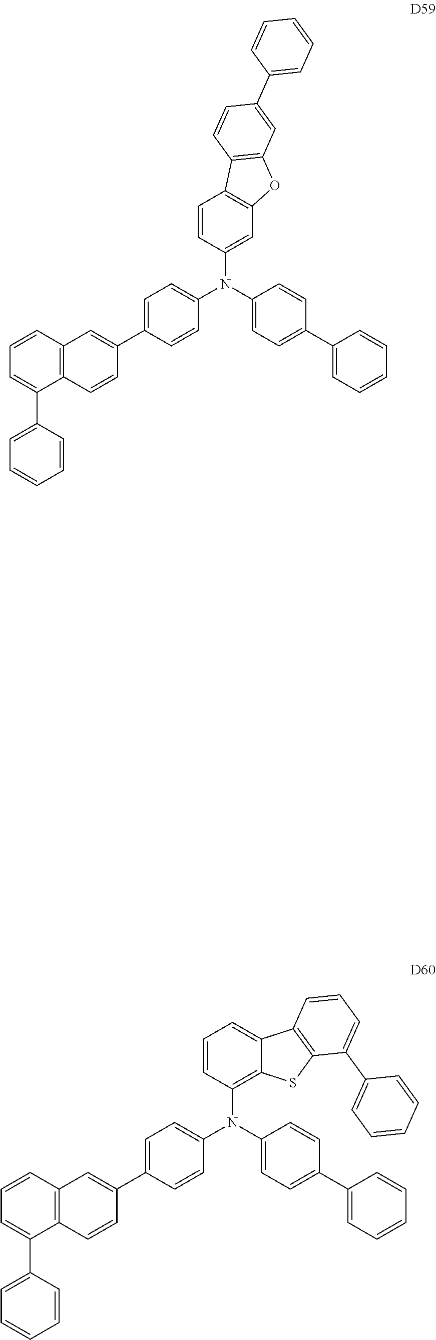 [SMILES: c1ccc(-c2ccc(N(c3ccc(-c4ccc5c(-c6ccccc6)cccc5c4)cc3)c3ccc4c(c3)oc3cc(-c5ccccc5)ccc34)cc2)cc1 ; c1ccc(-c2ccc(N(c3ccc(-c4ccc5c(-c6ccccc6)cccc5c4)cc3)c3cccc4c3sc3c(-c5ccccc5)cccc34)cc2)cc1]

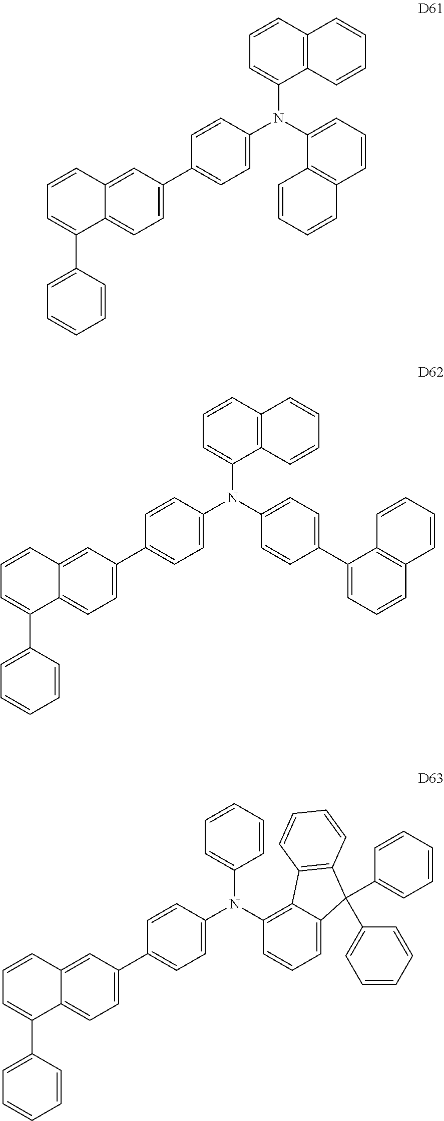 [SMILES: c1ccc(-c2cccc3cc(-c4ccc(N(c5cccc6ccccc56)c5cccc6ccccc56)cc4)ccc23)cc1 ; c1ccc(-c2cccc3cc(-c4ccc(N(c5ccc(-c6cccc7ccccc67)cc5)c5cccc6ccccc56)cc4)ccc23)cc1 ; c1ccc(-c2cccc3cc(-c4ccc(N(c5ccccc5)c5cccc6c5-c5ccccc5C6(c5ccccc5)c5ccccc5)cc4)ccc23)cc1]

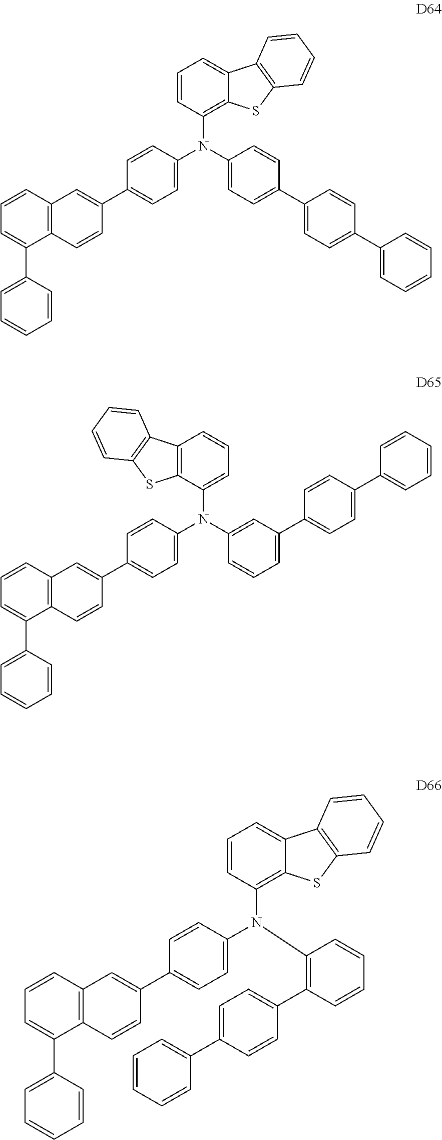 [SMILES: c1ccc(-c2ccc(-c3ccc(N(c4ccc(-c5ccc6c(-c7ccccc7)cccc6c5)cc4)c4cccc5c4sc4ccccc45)cc3)cc2)cc1 ; c1ccc(-c2ccc(-c3cccc(N(c4ccc(-c5ccc6c(-c7ccccc7)cccc6c5)cc4)c4cccc5c4sc4ccccc45)c3)cc2)cc1 ; c1ccc(-c2ccc(-c3ccccc3N(c3ccc(-c4ccc5c(-c6ccccc6)cccc5c4)cc3)c3cccc4c3sc3ccccc34)cc2)cc1]

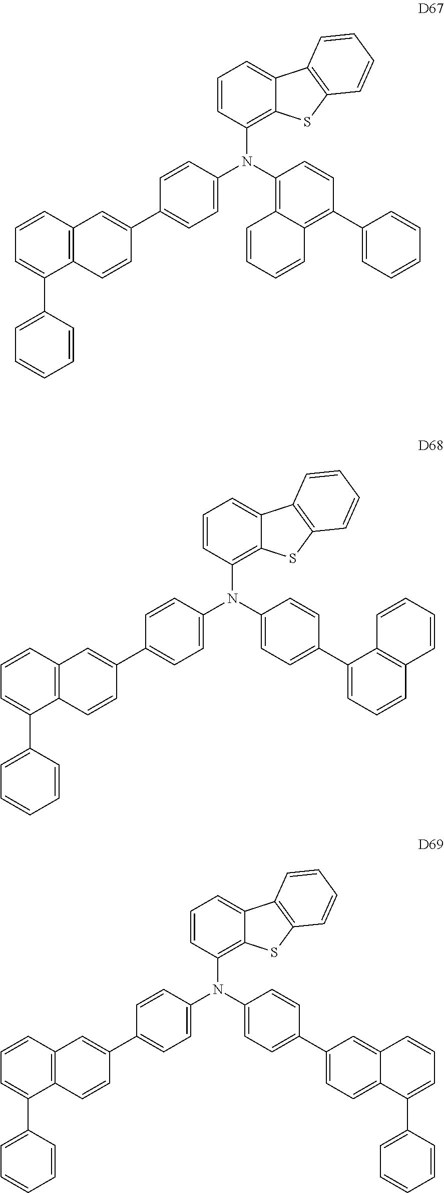 [SMILES: c1ccc(-c2cccc3cc(-c4ccc(N(c5ccc(-c6ccccc6)c6ccccc56)c5cccc6c5sc5ccccc56)cc4)ccc23)cc1 ; c1ccc(-c2cccc3cc(-c4ccc(N(c5ccc(-c6cccc7ccccc67)cc5)c5cccc6c5sc5ccccc56)cc4)ccc23)cc1 ; c1ccc(-c2cccc3cc(-c4ccc(N(c5ccc(-c6ccc7c(-c8ccccc8)cccc7c6)cc5)c5cccc6c5sc5ccccc56)cc4)ccc23)cc1]

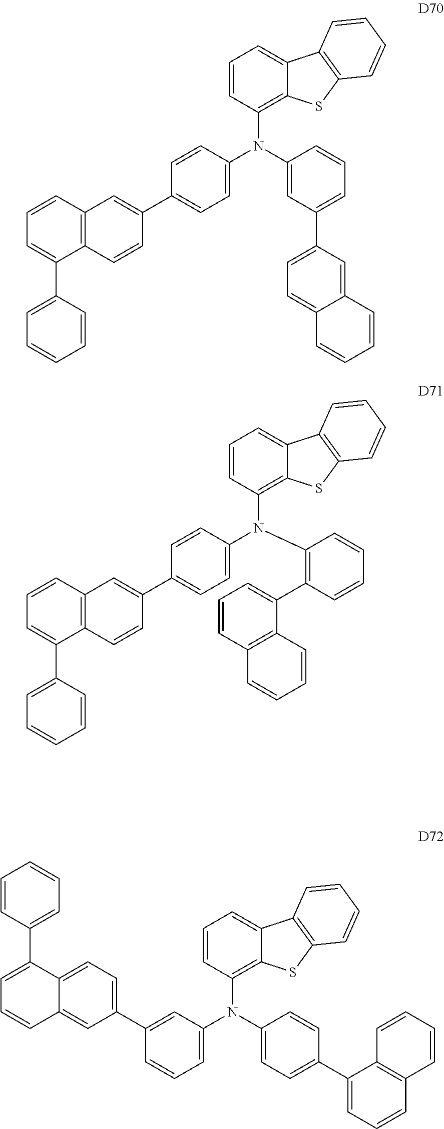

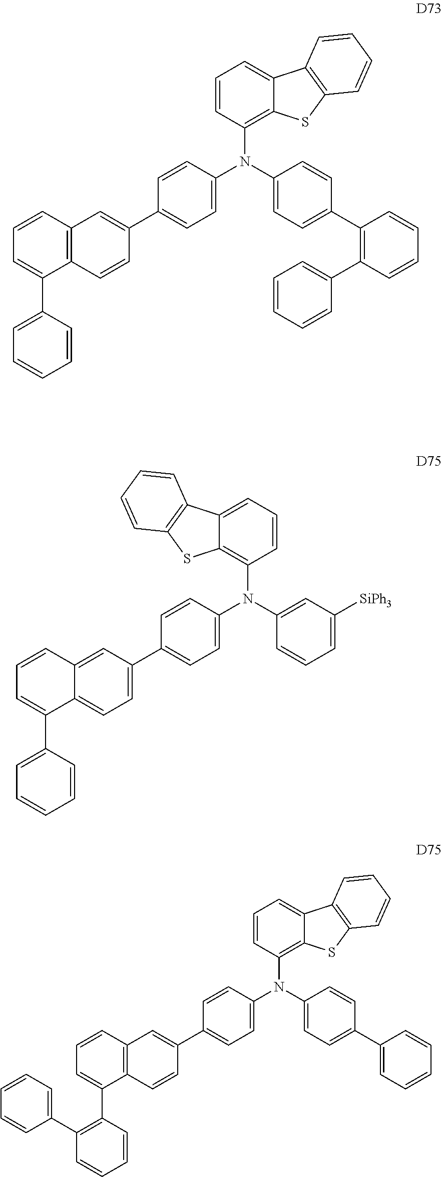

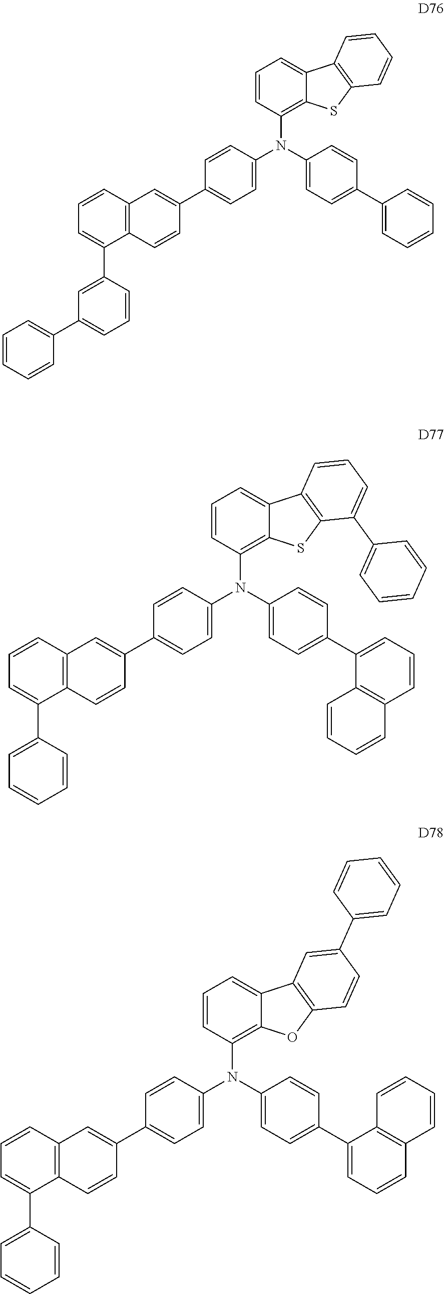 [SMILES: c1ccc(-c2ccc(N(c3ccc(-c4ccc5c(-c6cccc(-c7ccccc7)c6)cccc5c4)cc3)c3cccc4c3sc3ccccc34)cc2)cc1 ; c1ccc(-c2cccc3cc(-c4ccc(N(c5ccc(-c6cccc7ccccc67)cc5)c5cccc6c5sc5c(-c7ccccc7)cccc56)cc4)ccc23)cc1 ; c1ccc(-c2ccc3oc4c(N(c5ccc(-c6ccc7c(-c8ccccc8)cccc7c6)cc5)c5ccc(-c6cccc7ccccc67)cc5)cccc4c3c2)cc1]

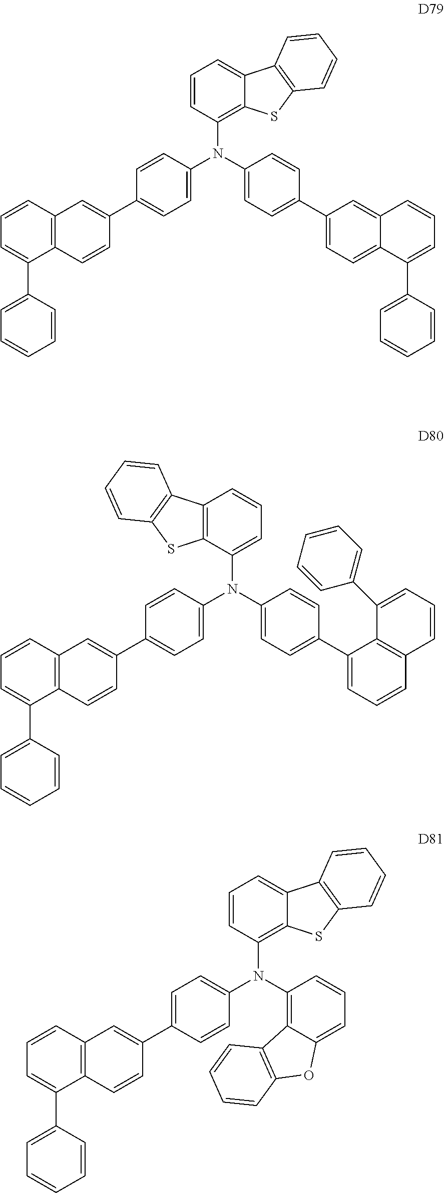 [SMILES: c1ccc(-c2cccc3cc(-c4ccc(N(c5ccc(-c6ccc7c(-c8ccccc8)cccc7c6)cc5)c5cccc6c5sc5ccccc56)cc4)ccc23)cc1 ; c1ccc(-c2cccc3cc(-c4ccc(N(c5ccc(-c6cccc7cccc(-c8ccccc8)c67)cc5)c5cccc6c5sc5ccccc56)cc4)ccc23)cc1 ; c1ccc(-c2cccc3cc(-c4ccc(N(c5cccc6c5sc5ccccc56)c5cccc6oc7ccccc7c56)cc4)ccc23)cc1]

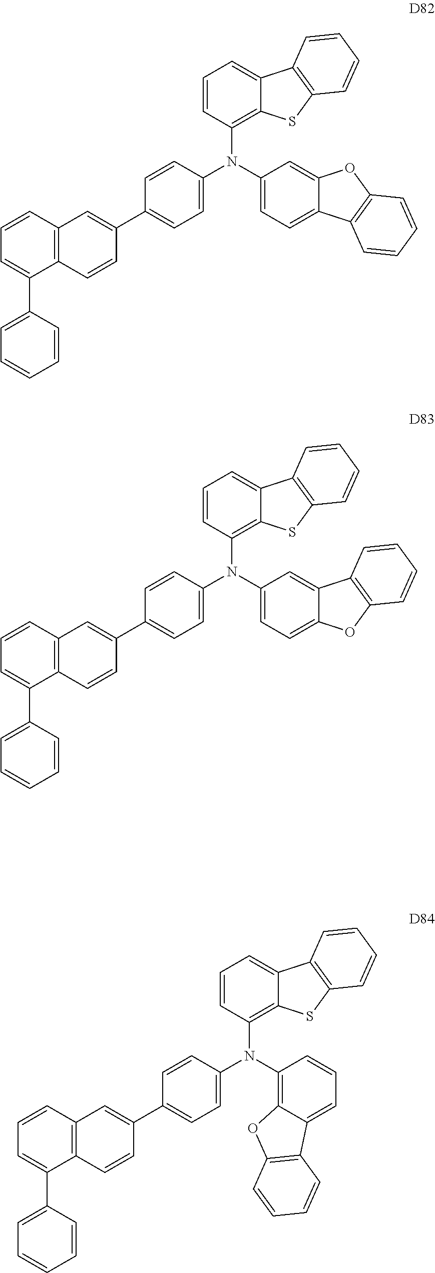 [SMILES: c1ccc(-c2cccc3cc(-c4ccc(N(c5ccc6c(c5)oc5ccccc56)c5cccc6c5sc5ccccc56)cc4)ccc23)cc1 ; c1ccc(-c2cccc3cc(-c4ccc(N(c5ccc6oc7ccccc7c6c5)c5cccc6c5sc5ccccc56)cc4)ccc23)cc1 ; c1ccc(-c2cccc3cc(-c4ccc(N(c5cccc6c5oc5ccccc56)c5cccc6c5sc5ccccc56)cc4)ccc23)cc1]

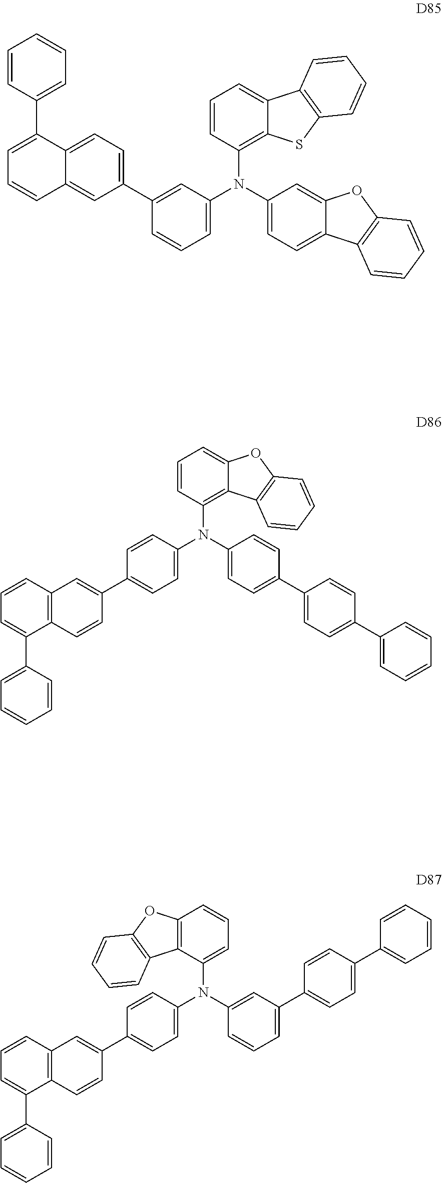 [SMILES: c1ccc(-c2cccc3cc(-c4cccc(N(c5ccc6c(c5)oc5ccccc56)c5cccc6c5sc5ccccc56)c4)ccc23)cc1 ; c1ccc(-c2ccc(-c3ccc(N(c4ccc(-c5ccc6c(-c7ccccc7)cccc6c5)cc4)c4cccc5oc6ccccc6c45)cc3)cc2)cc1 ; c1ccc(-c2ccc(-c3cccc(N(c4ccc(-c5ccc6c(-c7ccccc7)cccc6c5)cc4)c4cccc5oc6ccccc6c45)c3)cc2)cc1]

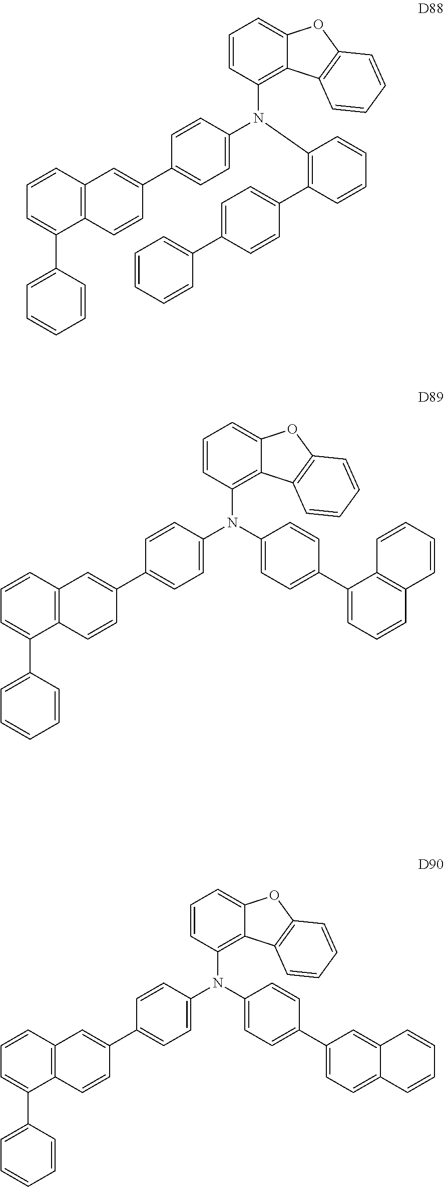 [SMILES: c1ccc(-c2ccc(-c3ccccc3N(c3ccc(-c4ccc5c(-c6ccccc6)cccc5c4)cc3)c3cccc4oc5ccccc5c34)cc2)cc1 ; c1ccc(-c2cccc3cc(-c4ccc(N(c5ccc(-c6cccc7ccccc67)cc5)c5cccc6oc7ccccc7c56)cc4)ccc23)cc1 ; c1ccc(-c2cccc3cc(-c4ccc(N(c5ccc(-c6ccc7ccccc7c6)cc5)c5cccc6oc7ccccc7c56)cc4)ccc23)cc1]

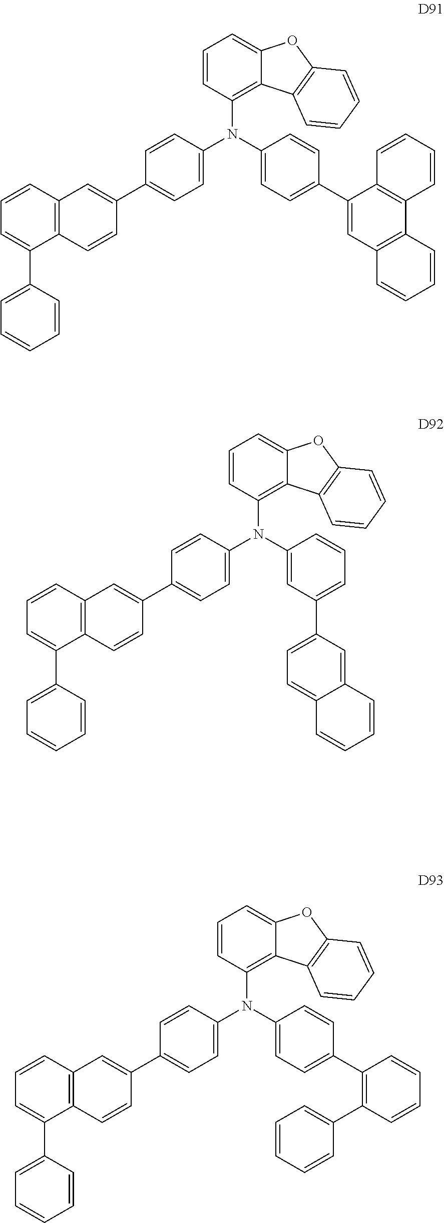 [SMILES: c1ccc(-c2cccc3cc(-c4ccc(N(c5ccc(-c6cc7ccccc7c7ccccc67)cc5)c5cccc6oc7ccccc7c56)cc4)ccc23)cc1 ; c1ccc(-c2cccc3cc(-c4ccc(N(c5cccc(-c6ccc7ccccc7c6)c5)c5cccc6oc7ccccc7c56)cc4)ccc23)cc1 ; c1ccc(-c2ccccc2-c2ccc(N(c3ccc(-c4ccc5c(-c6ccccc6)cccc5c4)cc3)c3cccc4oc5ccccc5c34)cc2)cc1]

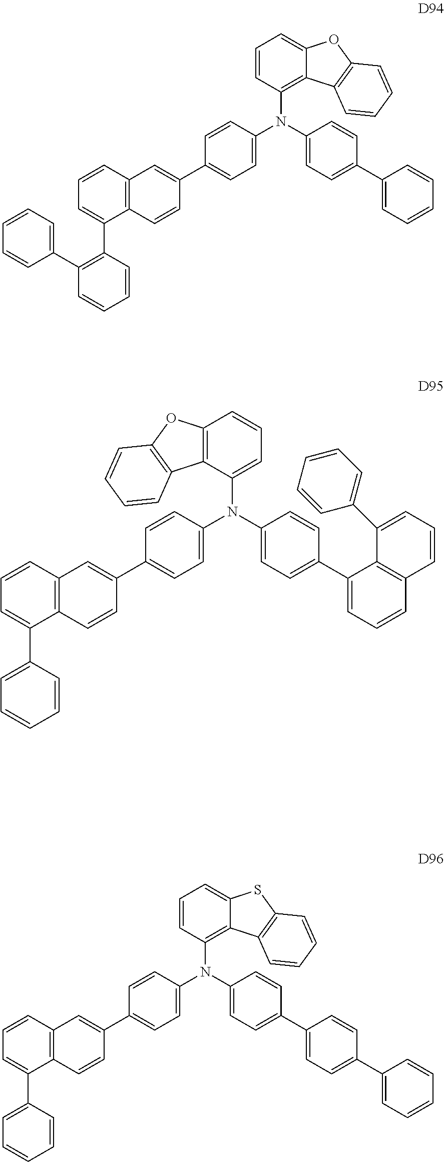 [SMILES: c1ccc(-c2ccc(N(c3ccc(-c4ccc5c(-c6ccccc6-c6ccccc6)cccc5c4)cc3)c3cccc4oc5ccccc5c34)cc2)cc1 ; c1ccc(-c2cccc3cc(-c4ccc(N(c5ccc(-c6cccc7cccc(-c8ccccc8)c67)cc5)c5cccc6oc7ccccc7c56)cc4)ccc23)cc1 ; c1ccc(-c2ccc(-c3ccc(N(c4ccc(-c5ccc6c(-c7ccccc7)cccc6c5)cc4)c4cccc5sc6ccccc6c45)cc3)cc2)cc1]

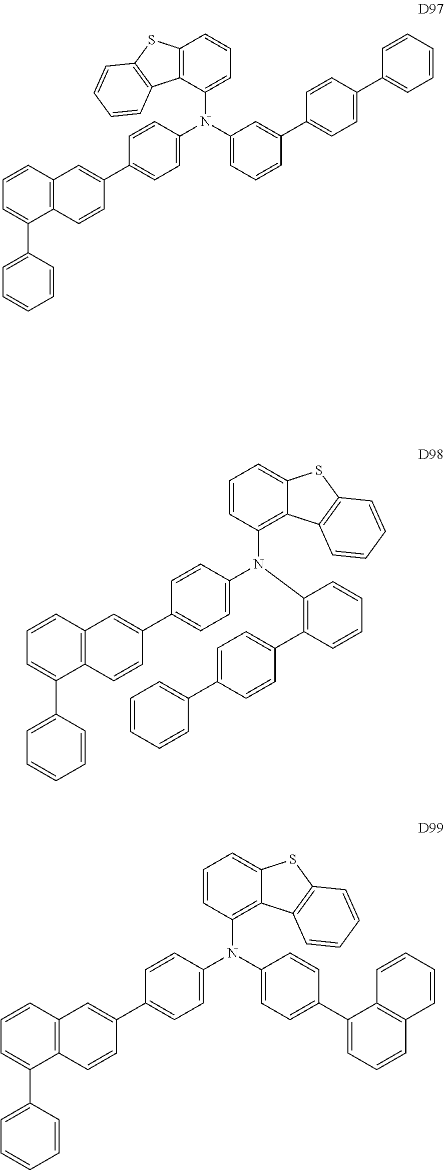 [SMILES: c1ccc(-c2ccc(-c3cccc(N(c4ccc(-c5ccc6c(-c7ccccc7)cccc6c5)cc4)c4cccc5sc6ccccc6c45)c3)cc2)cc1 ; c1ccc(-c2ccc(-c3ccccc3N(c3ccc(-c4ccc5c(-c6ccccc6)cccc5c4)cc3)c3cccc4sc5ccccc5c34)cc2)cc1 ; c1ccc(-c2cccc3cc(-c4ccc(N(c5ccc(-c6cccc7ccccc67)cc5)c5cccc6sc7ccccc7c56)cc4)ccc23)cc1]

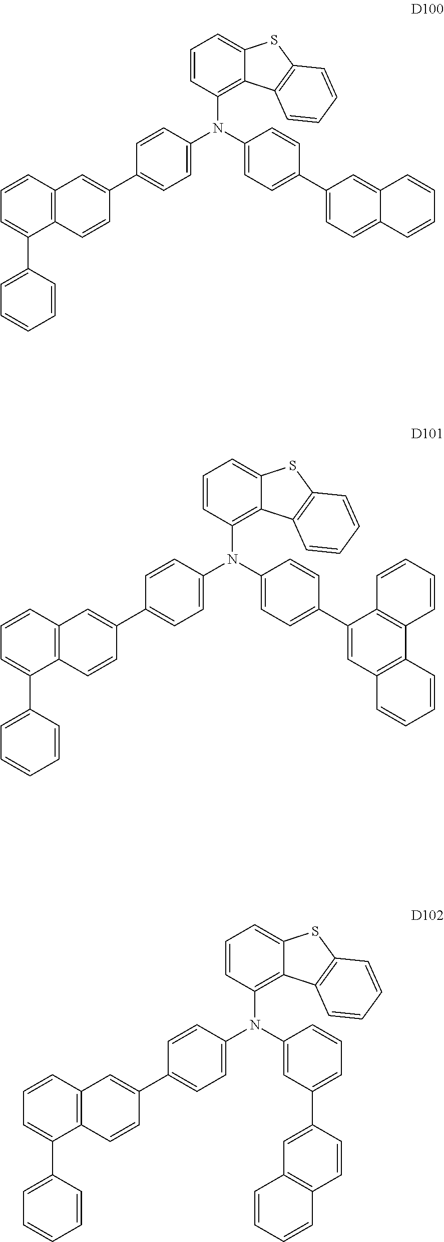 [SMILES: c1ccc(-c2cccc3cc(-c4ccc(N(c5ccc(-c6ccc7ccccc7c6)cc5)c5cccc6sc7ccccc7c56)cc4)ccc23)cc1 ; c1ccc(-c2cccc3cc(-c4ccc(N(c5ccc(-c6cc7ccccc7c7ccccc67)cc5)c5cccc6sc7ccccc7c56)cc4)ccc23)cc1 ; c1ccc(-c2cccc3cc(-c4ccc(N(c5cccc(-c6ccc7ccccc7c6)c5)c5cccc6sc7ccccc7c56)cc4)ccc23)cc1]

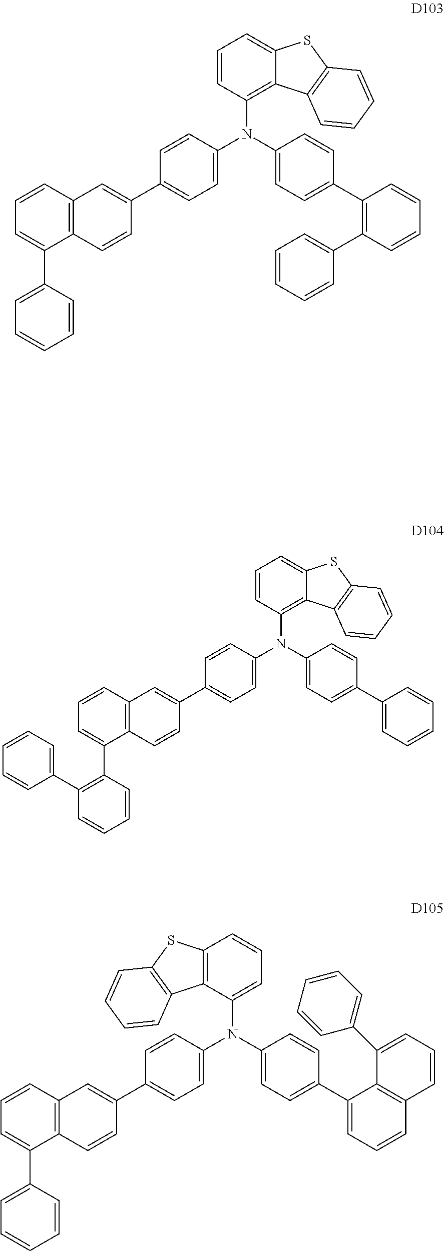 [SMILES: c1ccc(-c2ccccc2-c2ccc(N(c3ccc(-c4ccc5c(-c6ccccc6)cccc5c4)cc3)c3cccc4sc5ccccc5c34)cc2)cc1 ; c1ccc(-c2ccc(N(c3ccc(-c4ccc5c(-c6ccccc6-c6ccccc6)cccc5c4)cc3)c3cccc4sc5ccccc5c34)cc2)cc1 ; c1ccc(-c2cccc3cc(-c4ccc(N(c5ccc(-c6cccc7cccc(-c8ccccc8)c67)cc5)c5cccc6sc7ccccc7c56)cc4)ccc23)cc1]

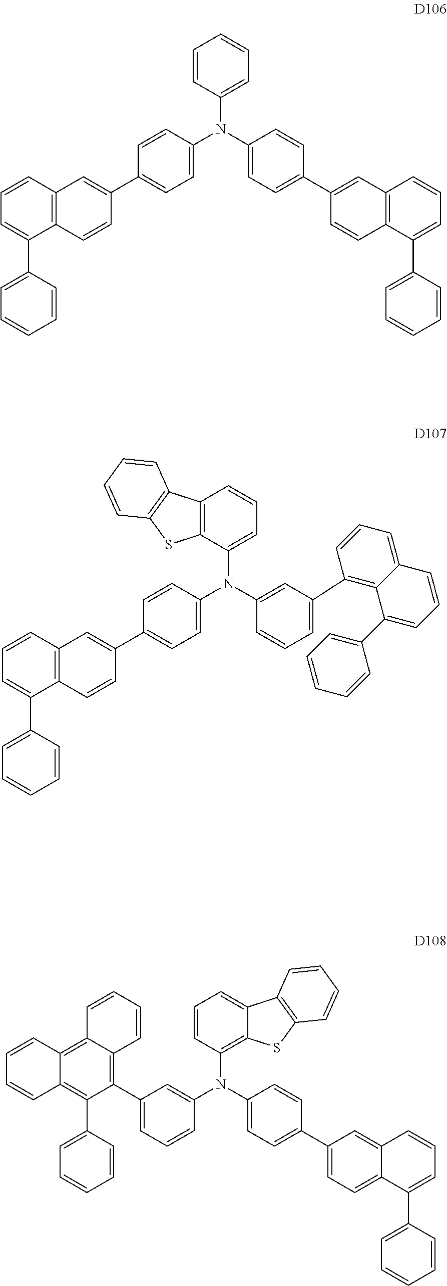 [SMILES: c1ccc(-c2cccc3cc(-c4ccc(N(c5ccccc5)c5ccc(-c6ccc7c(-c8ccccc8)cccc7c6)cc5)cc4)ccc23)cc1 ; c1ccc(-c2cccc3cc(-c4ccc(N(c5cccc(-c6cccc7cccc(-c8ccccc8)c67)c5)c5cccc6c5sc5ccccc56)cc4)ccc23)cc1 ; c1ccc(-c2cccc3cc(-c4ccc(N(c5cccc(-c6c(-c7ccccc7)c7ccccc7c7ccccc67)c5)c5cccc6c5sc5ccccc56)cc4)ccc23)cc1]

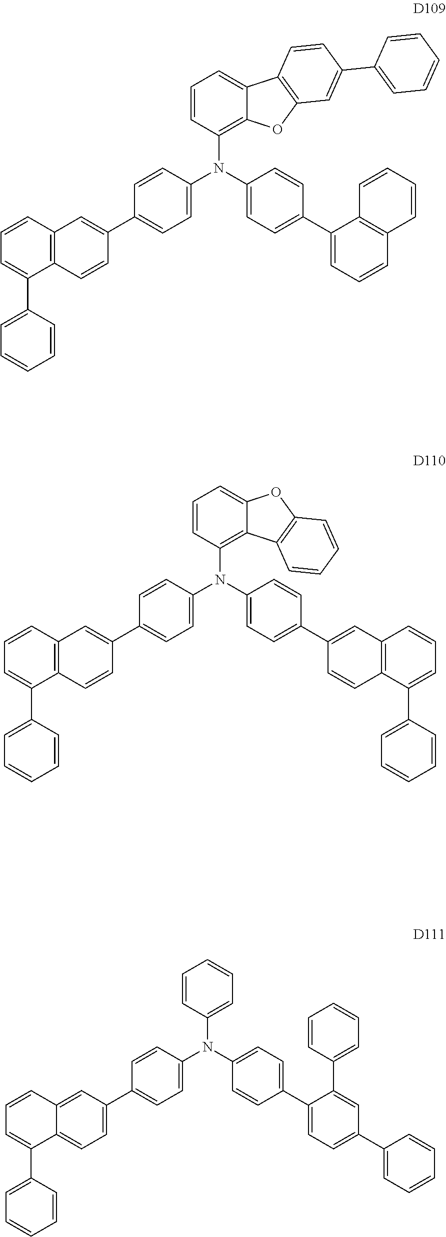 [SMILES: c1ccc(-c2ccc3c(c2)oc2c(N(c4ccc(-c5ccc6c(-c7ccccc7)cccc6c5)cc4)c4ccc(-c5cccc6ccccc56)cc4)cccc23)cc1 ; c1ccc(-c2cccc3cc(-c4ccc(N(c5ccc(-c6ccc7c(-c8ccccc8)cccc7c6)cc5)c5cccc6oc7ccccc7c56)cc4)ccc23)cc1 ; c1ccc(-c2ccc(-c3ccc(N(c4ccccc4)c4ccc(-c5ccc6c(-c7ccccc7)cccc6c5)cc4)cc3)c(-c3ccccc3)c2)cc1]

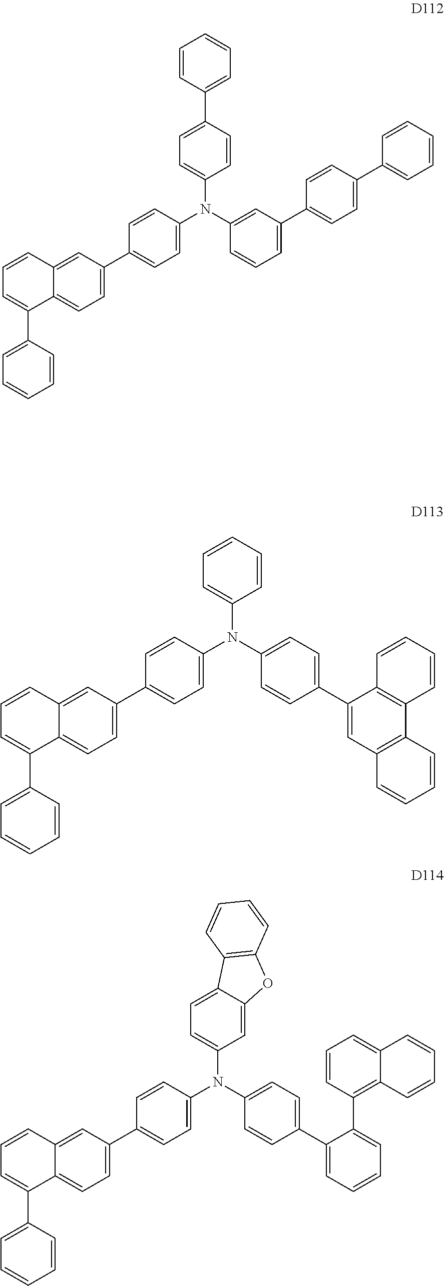 [SMILES: c1ccc(-c2ccc(-c3cccc(N(c4ccc(-c5ccccc5)cc4)c4ccc(-c5ccc6c(-c7ccccc7)cccc6c5)cc4)c3)cc2)cc1 ; c1ccc(-c2cccc3cc(-c4ccc(N(c5ccccc5)c5ccc(-c6cc7ccccc7c7ccccc67)cc5)cc4)ccc23)cc1 ; c1ccc(-c2cccc3cc(-c4ccc(N(c5ccc(-c6ccccc6-c6cccc7ccccc67)cc5)c5ccc6c(c5)oc5ccccc56)cc4)ccc23)cc1]

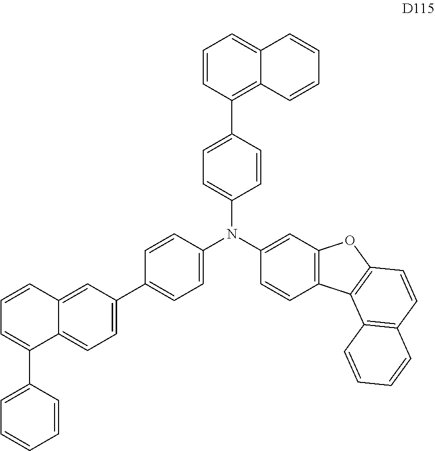 [SMILES: c1ccc(-c2cccc3cc(-c4ccc(N(c5ccc(-c6cccc7ccccc67)cc5)c5ccc6c(c5)oc5ccc7ccccc7c56)cc4)ccc23)cc1]

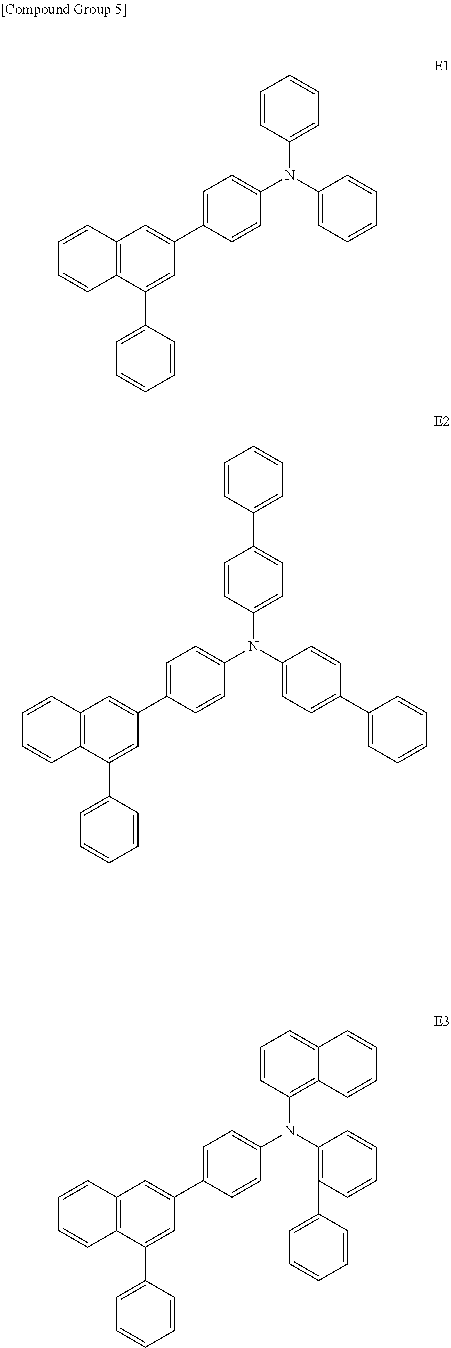 [SMILES: c1ccc(-c2cc(-c3ccc(N(c4ccccc4)c4ccccc4)cc3)cc3ccccc23)cc1 ; c1ccc(-c2ccc(N(c3ccc(-c4ccccc4)cc3)c3ccc(-c4cc(-c5ccccc5)c5ccccc5c4)cc3)cc2)cc1 ; c1ccc(-c2ccccc2N(c2ccc(-c3cc(-c4ccccc4)c4ccccc4c3)cc2)c2cccc3ccccc23)cc1]

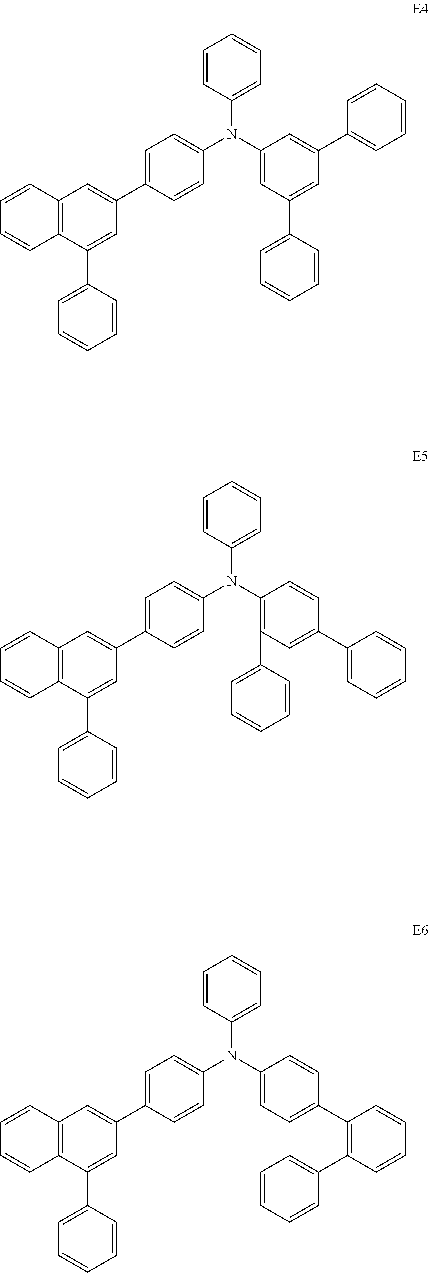 [SMILES: c1ccc(-c2cc(-c3ccccc3)cc(N(c3ccccc3)c3ccc(-c4cc(-c5ccccc5)c5ccccc5c4)cc3)c2)cc1 ; c1ccc(-c2ccc(N(c3ccccc3)c3ccc(-c4cc(-c5ccccc5)c5ccccc5c4)cc3)c(-c3ccccc3)c2)cc1 ; c1ccc(-c2ccccc2-c2ccc(N(c3ccccc3)c3ccc(-c4cc(-c5ccccc5)c5ccccc5c4)cc3)cc2)cc1]

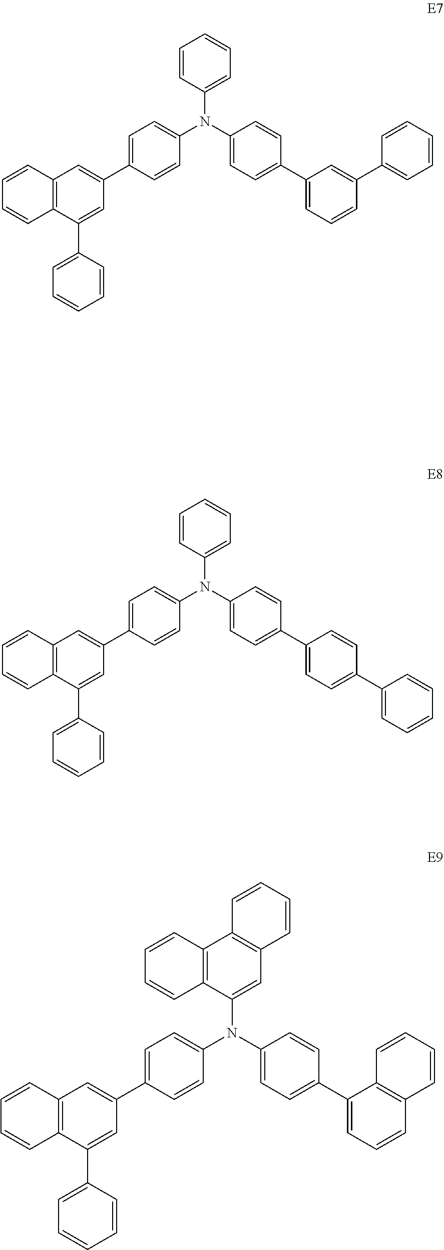 [SMILES: c1ccc(-c2cccc(-c3ccc(N(c4ccccc4)c4ccc(-c5cc(-c6ccccc6)c6ccccc6c5)cc4)cc3)c2)cc1 ; c1ccc(-c2ccc(-c3ccc(N(c4ccccc4)c4ccc(-c5cc(-c6ccccc6)c6ccccc6c5)cc4)cc3)cc2)cc1 ; c1ccc(-c2cc(-c3ccc(N(c4ccc(-c5cccc6ccccc56)cc4)c4cc5ccccc5c5ccccc45)cc3)cc3ccccc23)cc1]

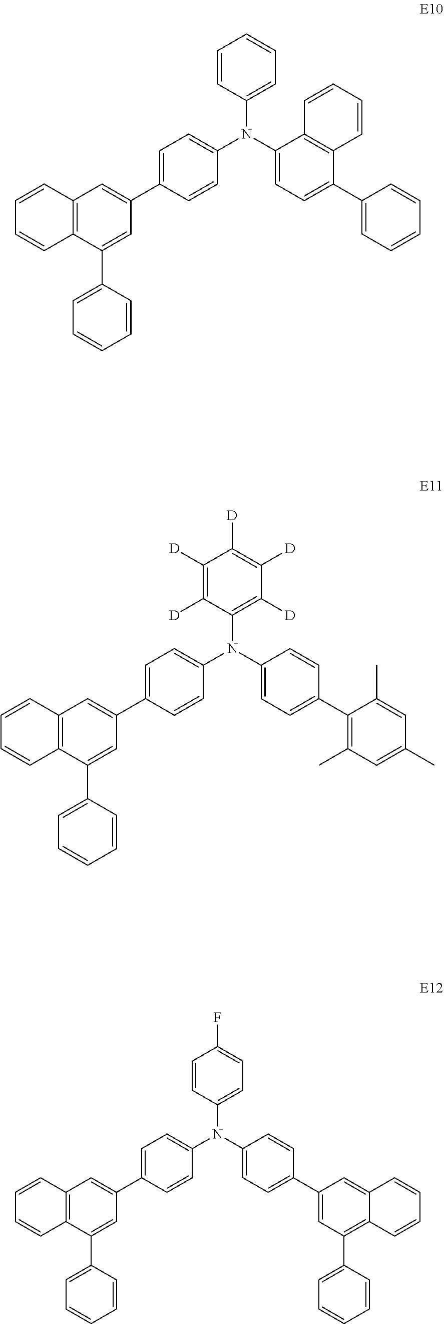 [SMILES: c1ccc(-c2cc(-c3ccc(N(c4ccccc4)c4ccc(-c5ccccc5)c5ccccc45)cc3)cc3ccccc23)cc1 ; [2H]c1c([2H])c([2H])c(N(c2ccc(-c3cc(-c4ccccc4)c4ccccc4c3)cc2)c2ccc(-c3c(C)cc(C)cc3C)cc2)c([2H])c1[2H] ; Fc1ccc(N(c2ccc(-c3cc(-c4ccccc4)c4ccccc4c3)cc2)c2ccc(-c3cc(-c4ccccc4)c4ccccc4c3)cc2)cc1]

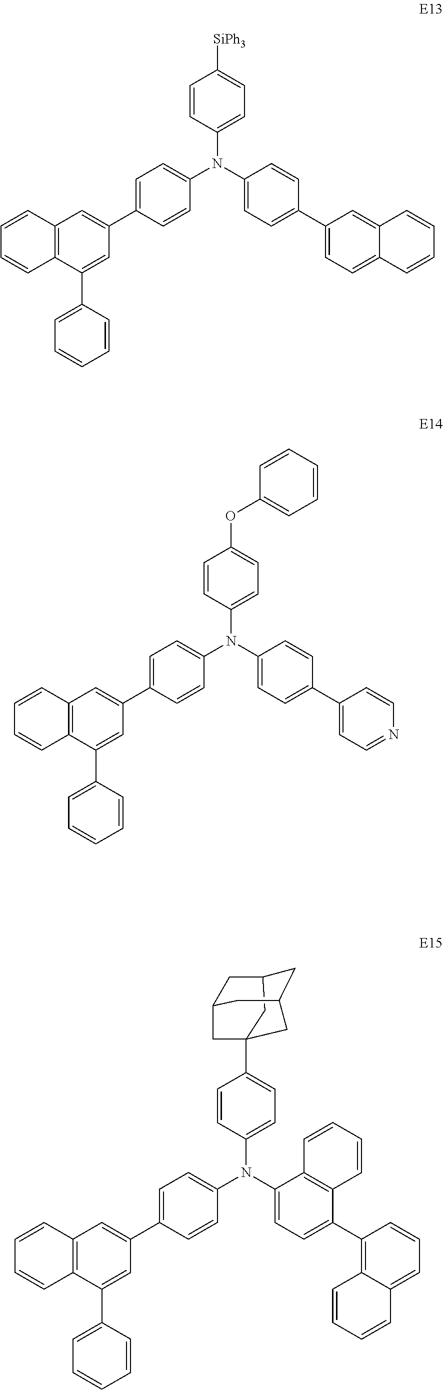

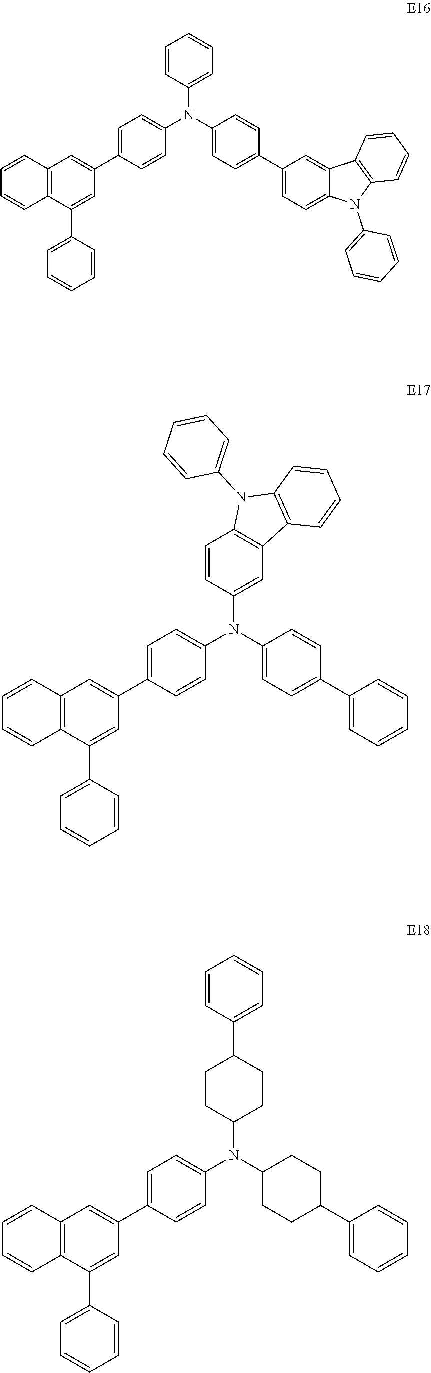 [SMILES: c1ccc(-c2cc(-c3ccc(N(c4ccccc4)c4ccc(-c5ccc6c(c5)c5ccccc5n6-c5ccccc5)cc4)cc3)cc3ccccc23)cc1 ; c1ccc(-c2ccc(N(c3ccc(-c4cc(-c5ccccc5)c5ccccc5c4)cc3)c3ccc4c(c3)c3ccccc3n4-c3ccccc3)cc2)cc1 ; c1ccc(-c2cc(-c3ccc(N(C4CCC(c5ccccc5)CC4)C4CCC(c5ccccc5)CC4)cc3)cc3ccccc23)cc1]

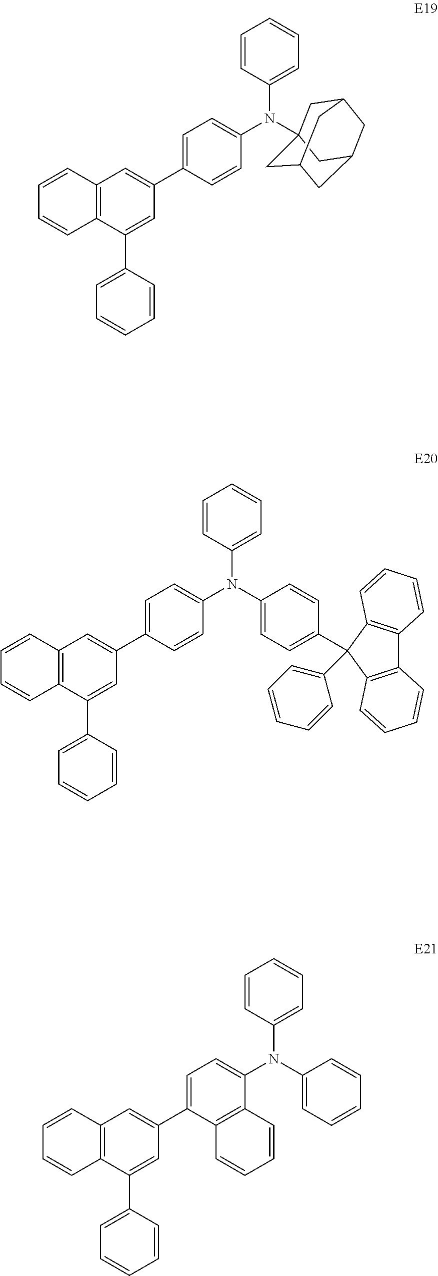 [SMILES: c1ccc(-c2cc(-c3ccc(N(c4ccccc4)C45CC6CC(CC(C6)C4)C5)cc3)cc3ccccc23)cc1 ; c1ccc(-c2cc(-c3ccc(N(c4ccccc4)c4ccc(C5(c6ccccc6)c6ccccc6-c6ccccc65)cc4)cc3)cc3ccccc23)cc1 ; c1ccc(-c2cc(-c3ccc(N(c4ccccc4)c4ccccc4)c4ccccc34)cc3ccccc23)cc1]

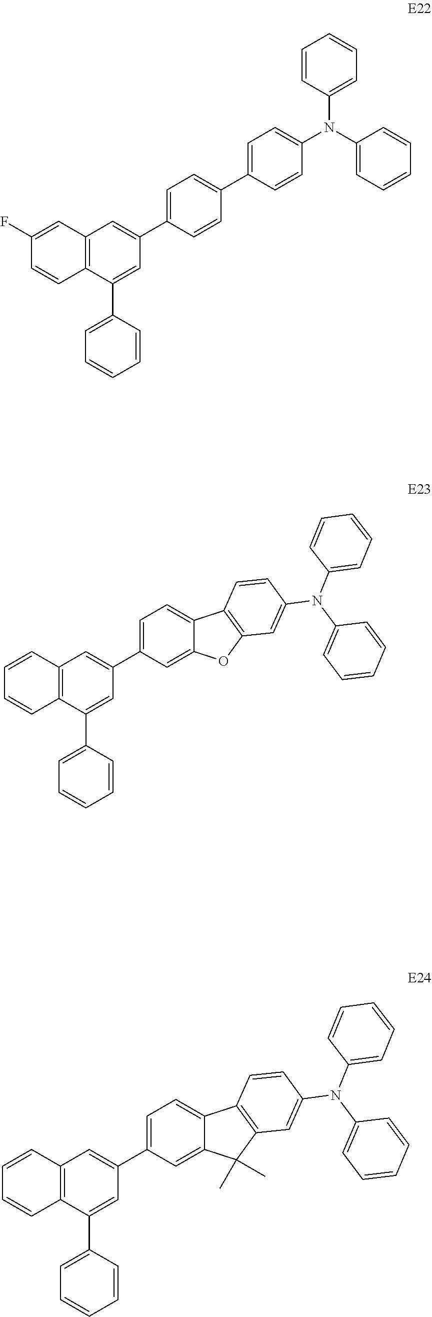 [SMILES: Fc1ccc2c(-c3ccccc3)cc(-c3ccc(-c4ccc(N(c5ccccc5)c5ccccc5)cc4)cc3)cc2c1 ; c1ccc(-c2cc(-c3ccc4c(c3)oc3cc(N(c5ccccc5)c5ccccc5)ccc34)cc3ccccc23)cc1 ; CC1(C)c2cc(-c3cc(-c4ccccc4)c4ccccc4c3)ccc2-c2ccc(N(c3ccccc3)c3ccccc3)cc21]

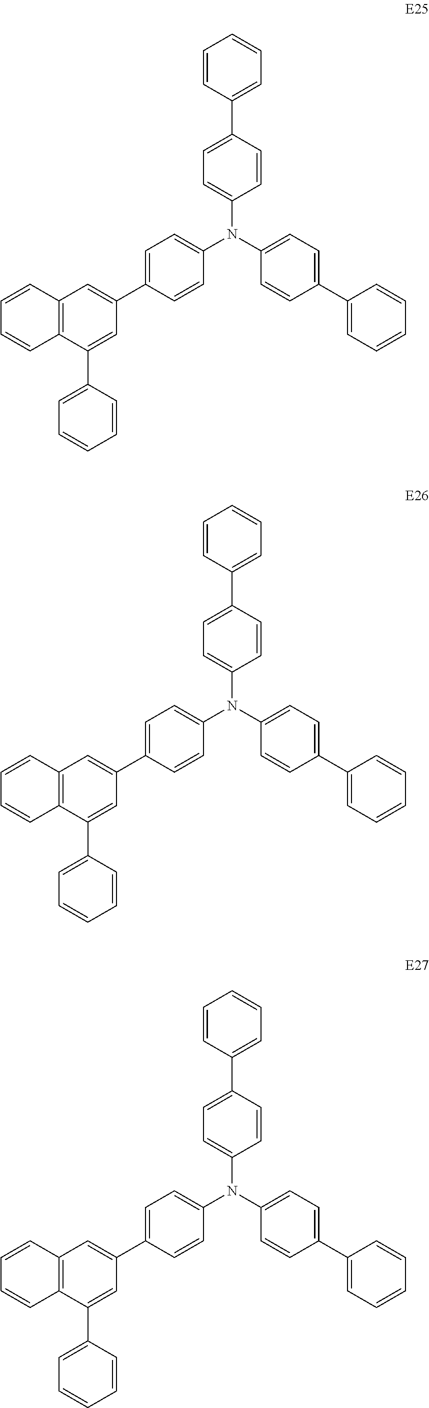 [SMILES: c1ccc(-c2ccc(N(c3ccc(-c4ccccc4)cc3)c3ccc(-c4cc(-c5ccccc5)c5ccccc5c4)cc3)cc2)cc1 ; c1ccc(-c2ccc(N(c3ccc(-c4ccccc4)cc3)c3ccc(-c4cc(-c5ccccc5)c5ccccc5c4)cc3)cc2)cc1 ; c1ccc(-c2ccc(N(c3ccc(-c4ccccc4)cc3)c3ccc(-c4cc(-c5ccccc5)c5ccccc5c4)cc3)cc2)cc1]

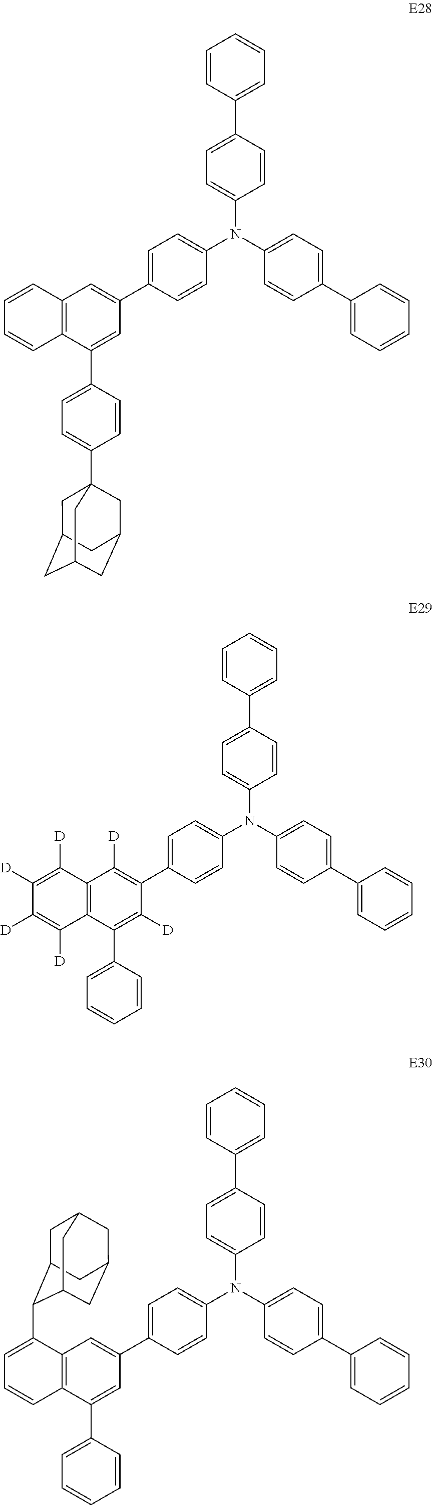 [SMILES: c1ccc(-c2ccc(N(c3ccc(-c4ccccc4)cc3)c3ccc(-c4cc(-c5ccc(C67CC8CC(CC(C8)C6)C7)cc5)c5ccccc5c4)cc3)cc2)cc1 ; [2H]c1c([2H])c([2H])c2c(-c3ccccc3)c([2H])c(-c3ccc(N(c4ccc(-c5ccccc5)cc4)c4ccc(-c5ccccc5)cc4)cc3)c([2H])c2c1[2H] ; c1ccc(-c2ccc(N(c3ccc(-c4ccccc4)cc3)c3ccc(-c4cc(-c5ccccc5)c5cccc(C6C7CC8CC(C7)CC6C8)c5c4)cc3)cc2)cc1]

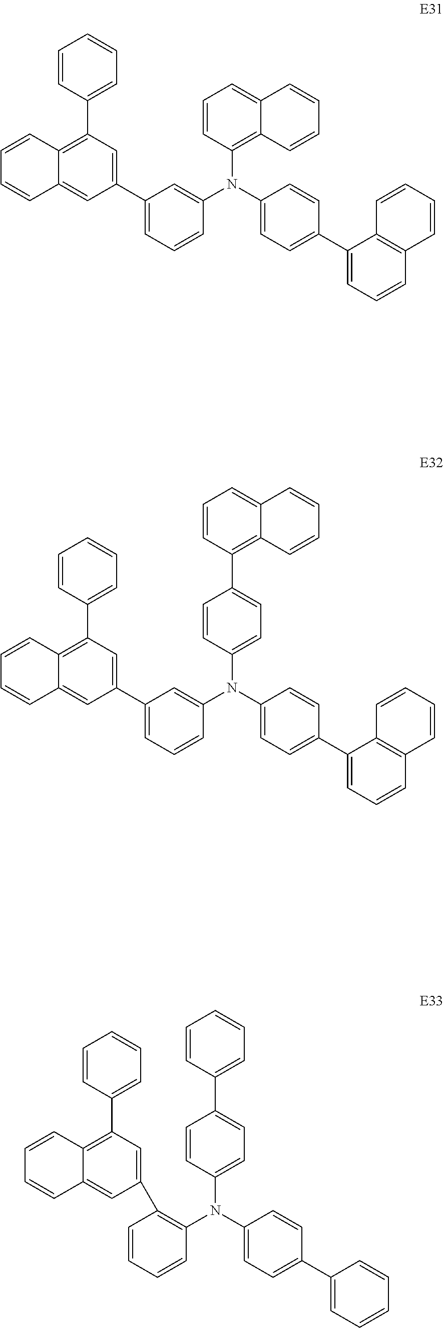 [SMILES: c1ccc(-c2cc(-c3cccc(N(c4ccc(-c5cccc6ccccc56)cc4)c4cccc5ccccc45)c3)cc3ccccc23)cc1 ; c1ccc(-c2cc(-c3cccc(N(c4ccc(-c5cccc6ccccc56)cc4)c4ccc(-c5cccc6ccccc56)cc4)c3)cc3ccccc23)cc1 ; c1ccc(-c2ccc(N(c3ccc(-c4ccccc4)cc3)c3ccccc3-c3cc(-c4ccccc4)c4ccccc4c3)cc2)cc1]

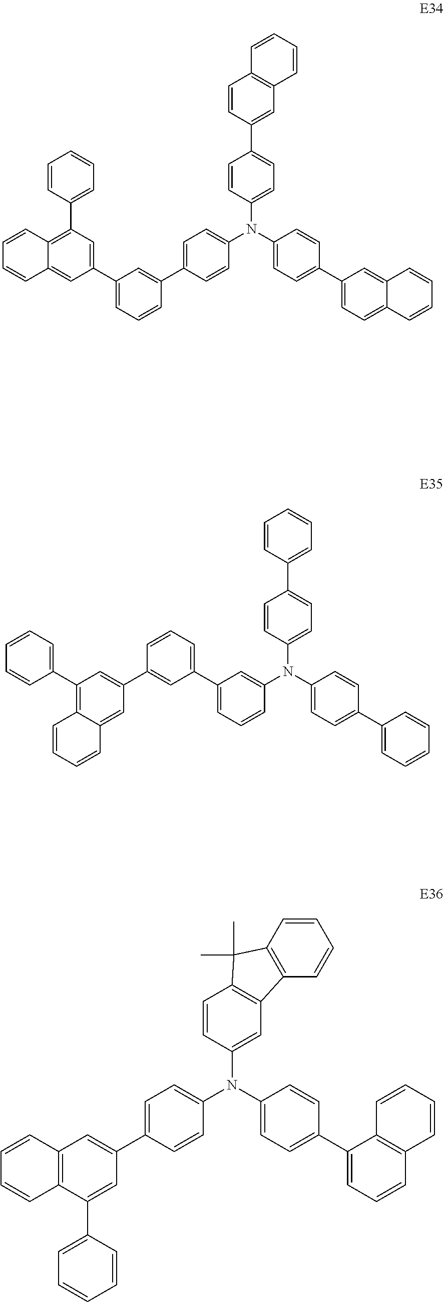 [SMILES: c1ccc(-c2cc(-c3cccc(-c4ccc(N(c5ccc(-c6ccc7ccccc7c6)cc5)c5ccc(-c6ccc7ccccc7c6)cc5)cc4)c3)cc3ccccc23)cc1 ; c1ccc(-c2ccc(N(c3ccc(-c4ccccc4)cc3)c3cccc(-c4cccc(-c5cc(-c6ccccc6)c6ccccc6c5)c4)c3)cc2)cc1 ; CC1(C)c2ccccc2-c2cc(N(c3ccc(-c4cc(-c5ccccc5)c5ccccc5c4)cc3)c3ccc(-c4cccc5ccccc45)cc3)ccc21]

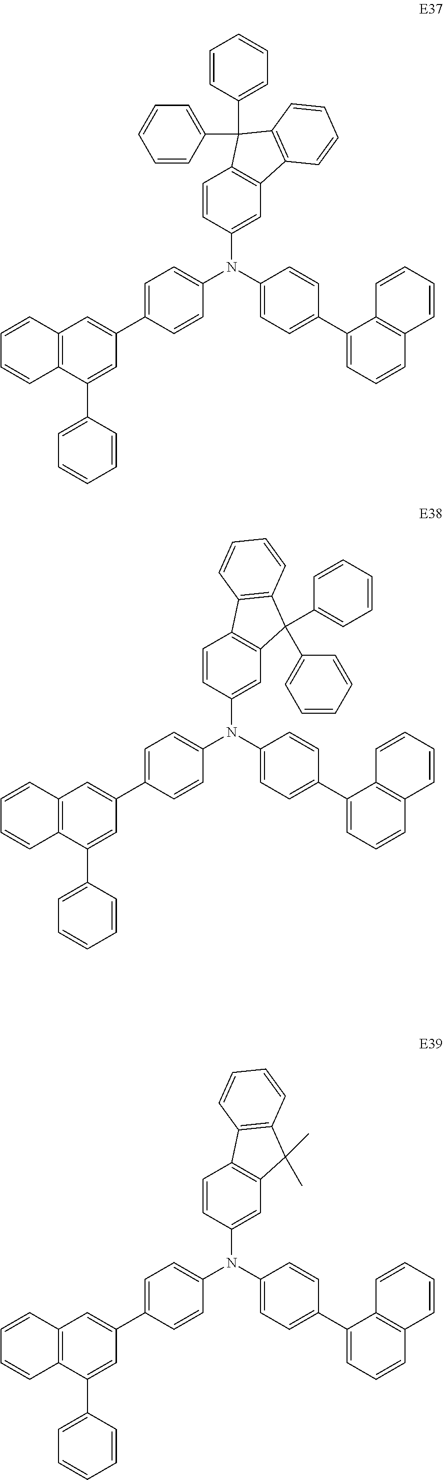 [SMILES: c1ccc(-c2cc(-c3ccc(N(c4ccc(-c5cccc6ccccc56)cc4)c4ccc5c(c4)-c4ccccc4C5(c4ccccc4)c4ccccc4)cc3)cc3ccccc23)cc1 ; c1ccc(-c2cc(-c3ccc(N(c4ccc(-c5cccc6ccccc56)cc4)c4ccc5c(c4)C(c4ccccc4)(c4ccccc4)c4ccccc4-5)cc3)cc3ccccc23)cc1 ; CC1(C)c2ccccc2-c2ccc(N(c3ccc(-c4cc(-c5ccccc5)c5ccccc5c4)cc3)c3ccc(-c4cccc5ccccc45)cc3)cc21]

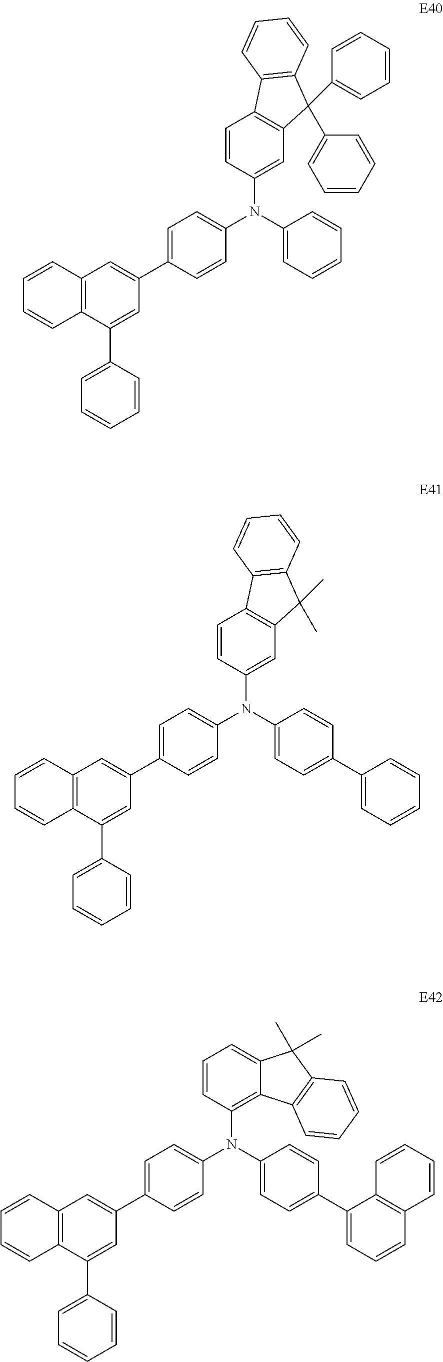 [SMILES: c1ccc(-c2cc(-c3ccc(N(c4ccccc4)c4ccc5c(c4)C(c4ccccc4)(c4ccccc4)c4ccccc4-5)cc3)cc3ccccc23)cc1 ; CC1(C)c2ccccc2-c2ccc(N(c3ccc(-c4ccccc4)cc3)c3ccc(-c4cc(-c5ccccc5)c5ccccc5c4)cc3)cc21 ; CC1(C)c2ccccc2-c2c(N(c3ccc(-c4cc(-c5ccccc5)c5ccccc5c4)cc3)c3ccc(-c4cccc5ccccc45)cc3)cccc21]

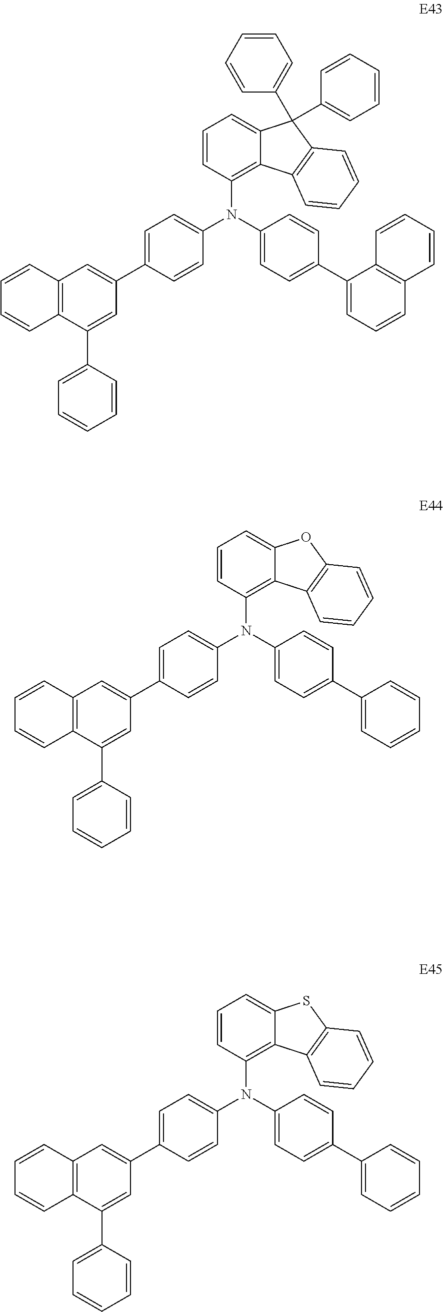 [SMILES: c1ccc(-c2cc(-c3ccc(N(c4ccc(-c5cccc6ccccc56)cc4)c4cccc5c4-c4ccccc4C5(c4ccccc4)c4ccccc4)cc3)cc3ccccc23)cc1 ; c1ccc(-c2ccc(N(c3ccc(-c4cc(-c5ccccc5)c5ccccc5c4)cc3)c3cccc4oc5ccccc5c34)cc2)cc1 ; c1ccc(-c2ccc(N(c3ccc(-c4cc(-c5ccccc5)c5ccccc5c4)cc3)c3cccc4sc5ccccc5c34)cc2)cc1]

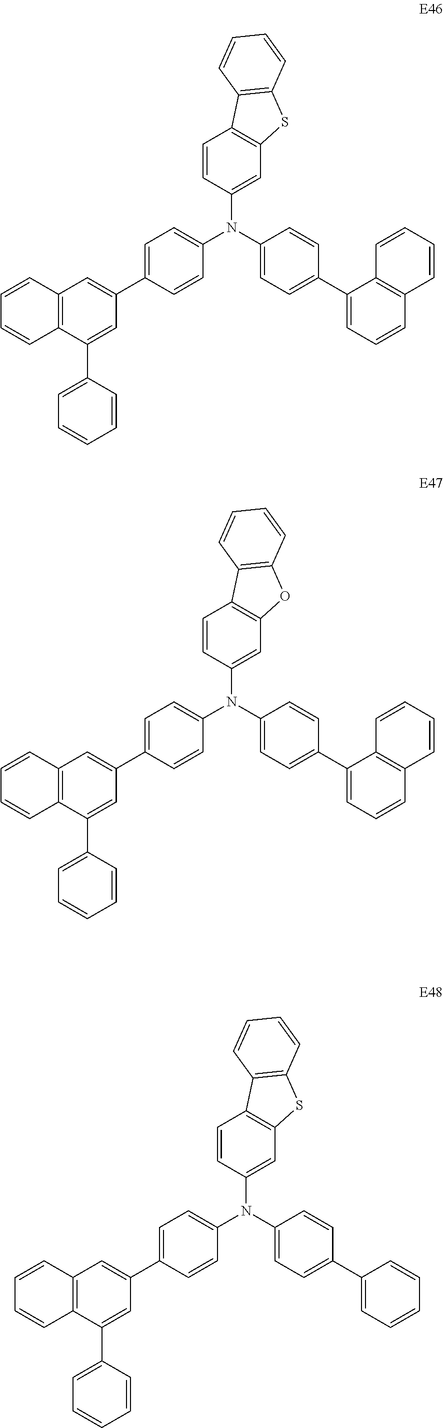 [SMILES: c1ccc(-c2cc(-c3ccc(N(c4ccc(-c5cccc6ccccc56)cc4)c4ccc5c(c4)sc4ccccc45)cc3)cc3ccccc23)cc1 ; c1ccc(-c2cc(-c3ccc(N(c4ccc(-c5cccc6ccccc56)cc4)c4ccc5c(c4)oc4ccccc45)cc3)cc3ccccc23)cc1 ; c1ccc(-c2ccc(N(c3ccc(-c4cc(-c5ccccc5)c5ccccc5c4)cc3)c3ccc4c(c3)sc3ccccc34)cc2)cc1]

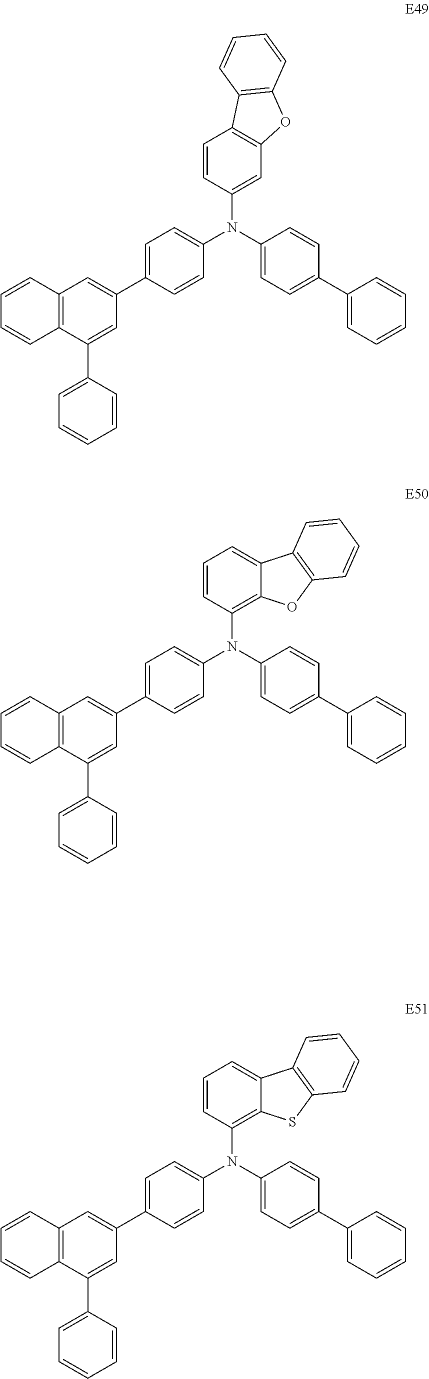 [SMILES: c1ccc(-c2ccc(N(c3ccc(-c4cc(-c5ccccc5)c5ccccc5c4)cc3)c3ccc4c(c3)oc3ccccc34)cc2)cc1 ; c1ccc(-c2ccc(N(c3ccc(-c4cc(-c5ccccc5)c5ccccc5c4)cc3)c3cccc4c3oc3ccccc34)cc2)cc1 ; c1ccc(-c2ccc(N(c3ccc(-c4cc(-c5ccccc5)c5ccccc5c4)cc3)c3cccc4c3sc3ccccc34)cc2)cc1]

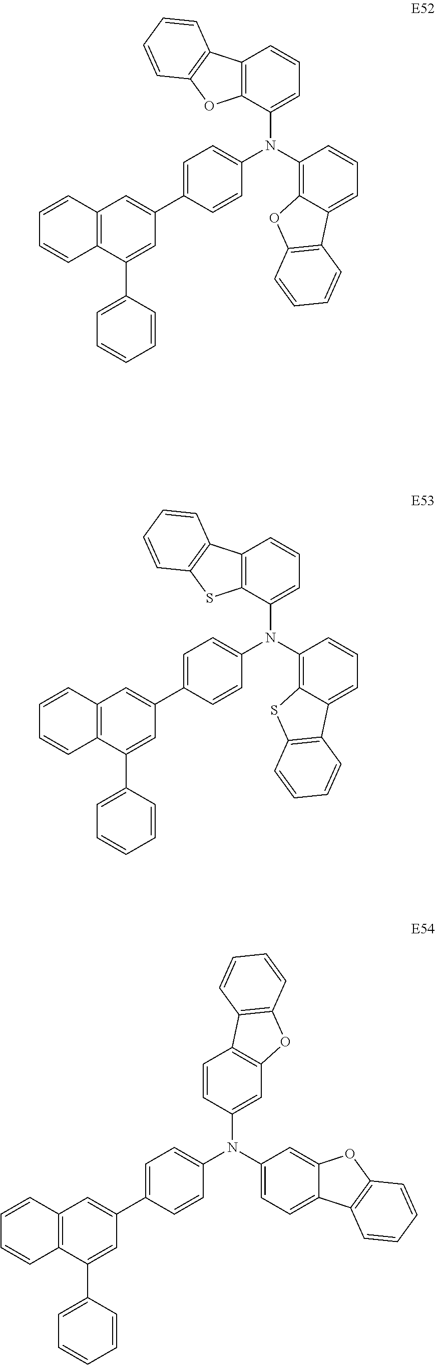 [SMILES: c1ccc(-c2cc(-c3ccc(N(c4cccc5c4oc4ccccc45)c4cccc5c4oc4ccccc45)cc3)cc3ccccc23)cc1 ; c1ccc(-c2cc(-c3ccc(N(c4cccc5c4sc4ccccc45)c4cccc5c4sc4ccccc45)cc3)cc3ccccc23)cc1 ; c1ccc(-c2cc(-c3ccc(N(c4ccc5c(c4)oc4ccccc45)c4ccc5c(c4)oc4ccccc45)cc3)cc3ccccc23)cc1]

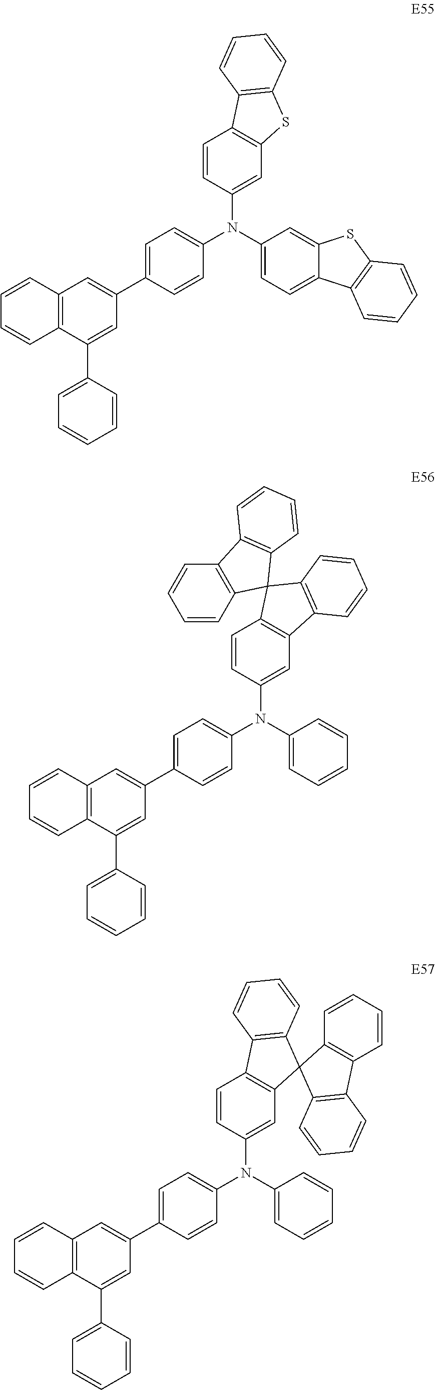 [SMILES: c1ccc(-c2cc(-c3ccc(N(c4ccc5c(c4)sc4ccccc45)c4ccc5c(c4)sc4ccccc45)cc3)cc3ccccc23)cc1 ; c1ccc(-c2cc(-c3ccc(N(c4ccccc4)c4ccc5c(c4)-c4ccccc4C54c5ccccc5-c5ccccc54)cc3)cc3ccccc23)cc1 ; c1ccc(-c2cc(-c3ccc(N(c4ccccc4)c4ccc5c(c4)C4(c6ccccc6-c6ccccc64)c4ccccc4-5)cc3)cc3ccccc23)cc1]

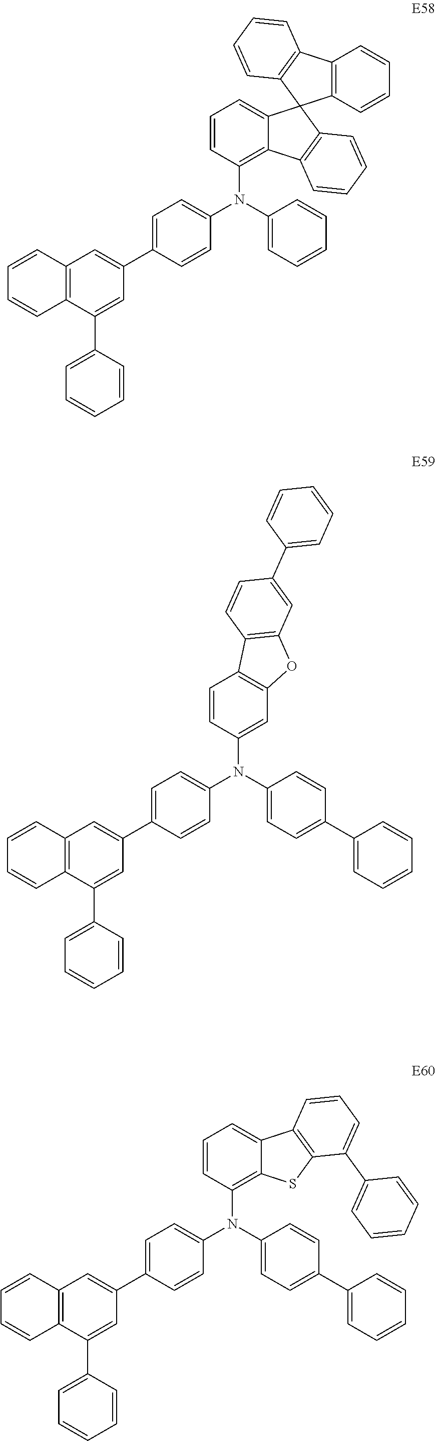 [SMILES: c1ccc(-c2cc(-c3ccc(N(c4ccccc4)c4cccc5c4-c4ccccc4C54c5ccccc5-c5ccccc54)cc3)cc3ccccc23)cc1 ; c1ccc(-c2ccc(N(c3ccc(-c4cc(-c5ccccc5)c5ccccc5c4)cc3)c3ccc4c(c3)oc3cc(-c5ccccc5)ccc34)cc2)cc1 ; c1ccc(-c2ccc(N(c3ccc(-c4cc(-c5ccccc5)c5ccccc5c4)cc3)c3cccc4c3sc3c(-c5ccccc5)cccc34)cc2)cc1]

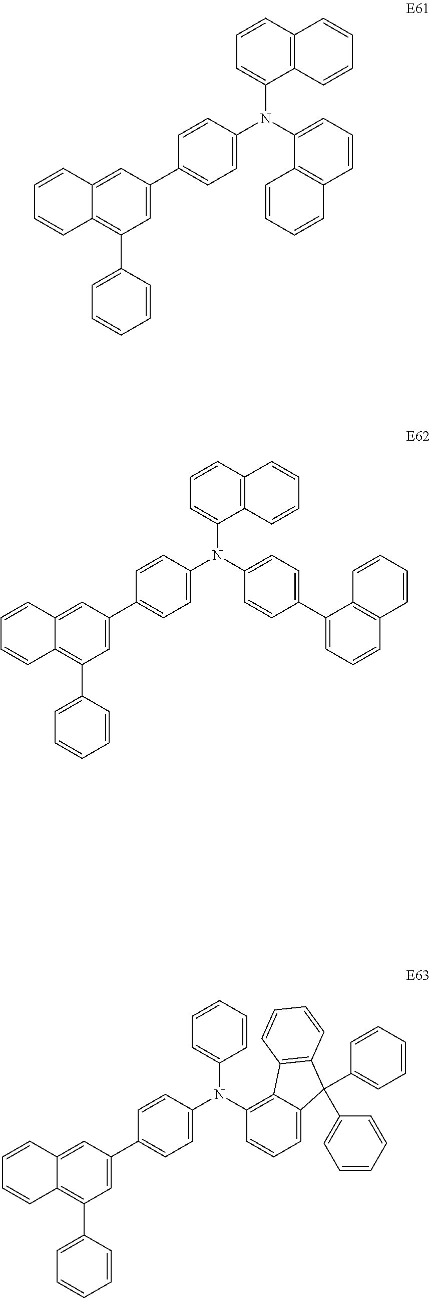 [SMILES: c1ccc(-c2cc(-c3ccc(N(c4cccc5ccccc45)c4cccc5ccccc45)cc3)cc3ccccc23)cc1 ; c1ccc(-c2cc(-c3ccc(N(c4ccc(-c5cccc6ccccc56)cc4)c4cccc5ccccc45)cc3)cc3ccccc23)cc1 ; c1ccc(-c2cc(-c3ccc(N(c4ccccc4)c4cccc5c4-c4ccccc4C5(c4ccccc4)c4ccccc4)cc3)cc3ccccc23)cc1]

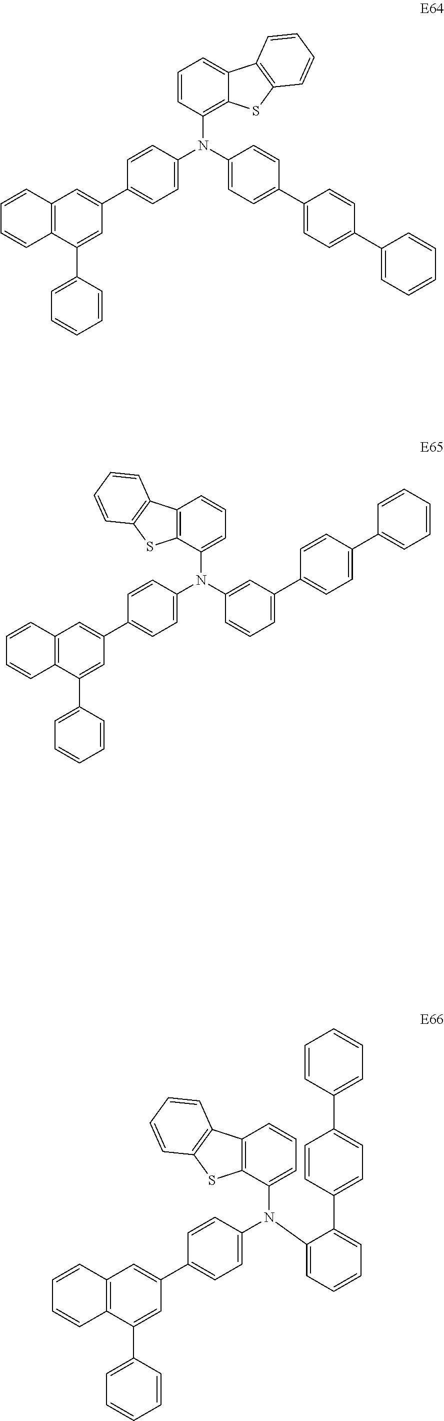 [SMILES: c1ccc(-c2ccc(-c3ccc(N(c4ccc(-c5cc(-c6ccccc6)c6ccccc6c5)cc4)c4cccc5c4sc4ccccc45)cc3)cc2)cc1 ; c1ccc(-c2ccc(-c3cccc(N(c4ccc(-c5cc(-c6ccccc6)c6ccccc6c5)cc4)c4cccc5c4sc4ccccc45)c3)cc2)cc1 ; c1ccc(-c2ccc(-c3ccccc3N(c3ccc(-c4cc(-c5ccccc5)c5ccccc5c4)cc3)c3cccc4c3sc3ccccc34)cc2)cc1]

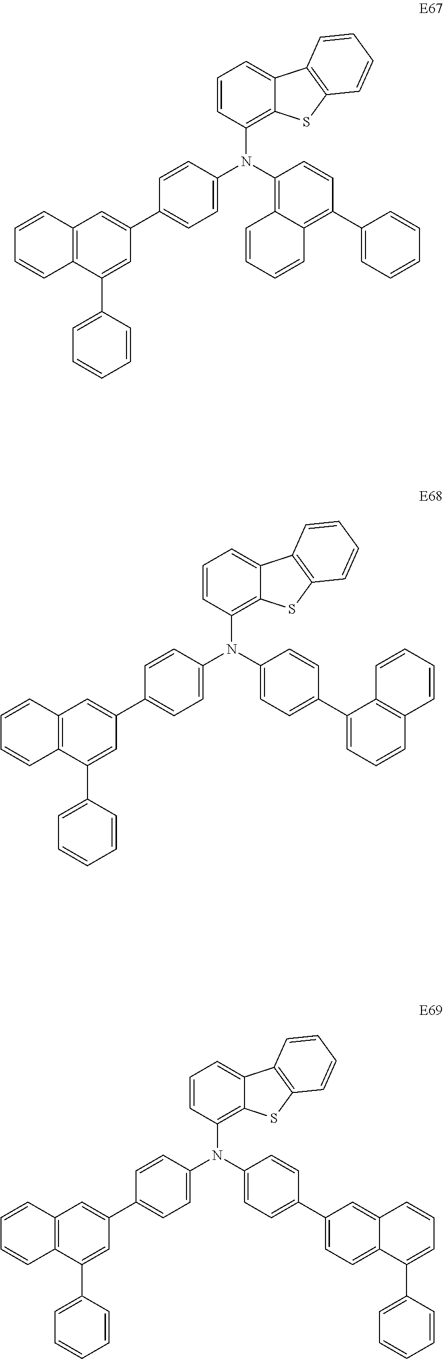 [SMILES: c1ccc(-c2cc(-c3ccc(N(c4ccc(-c5ccccc5)c5ccccc45)c4cccc5c4sc4ccccc45)cc3)cc3ccccc23)cc1 ; c1ccc(-c2cc(-c3ccc(N(c4ccc(-c5cccc6ccccc56)cc4)c4cccc5c4sc4ccccc45)cc3)cc3ccccc23)cc1 ; c1ccc(-c2cccc3cc(-c4ccc(N(c5ccc(-c6cc(-c7ccccc7)c7ccccc7c6)cc5)c5cccc6c5sc5ccccc56)cc4)ccc23)cc1]

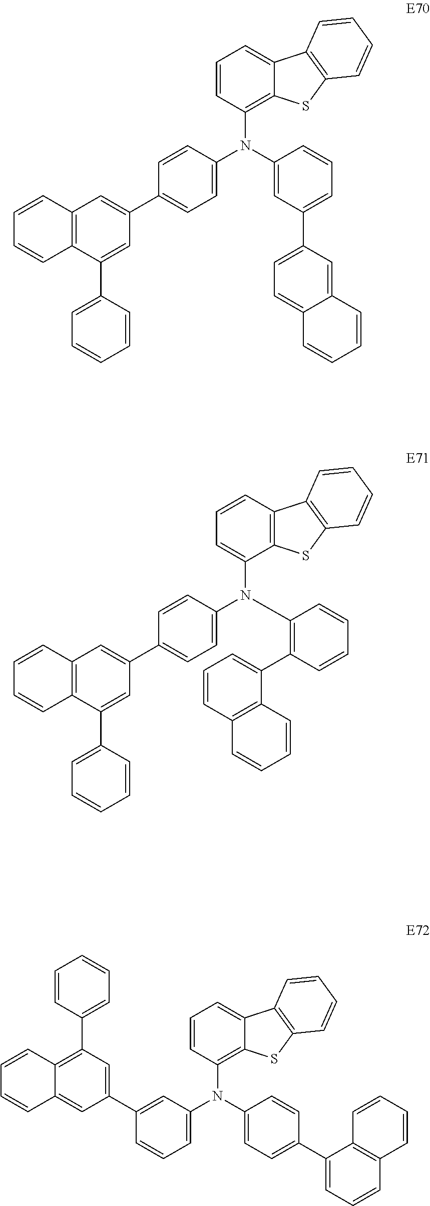 [SMILES: c1ccc(-c2cc(-c3ccc(N(c4cccc(-c5ccc6ccccc6c5)c4)c4cccc5c4sc4ccccc45)cc3)cc3ccccc23)cc1 ; c1ccc(-c2cc(-c3ccc(N(c4ccccc4-c4cccc5ccccc45)c4cccc5c4sc4ccccc45)cc3)cc3ccccc23)cc1 ; c1ccc(-c2cc(-c3cccc(N(c4ccc(-c5cccc6ccccc56)cc4)c4cccc5c4sc4ccccc45)c3)cc3ccccc23)cc1]

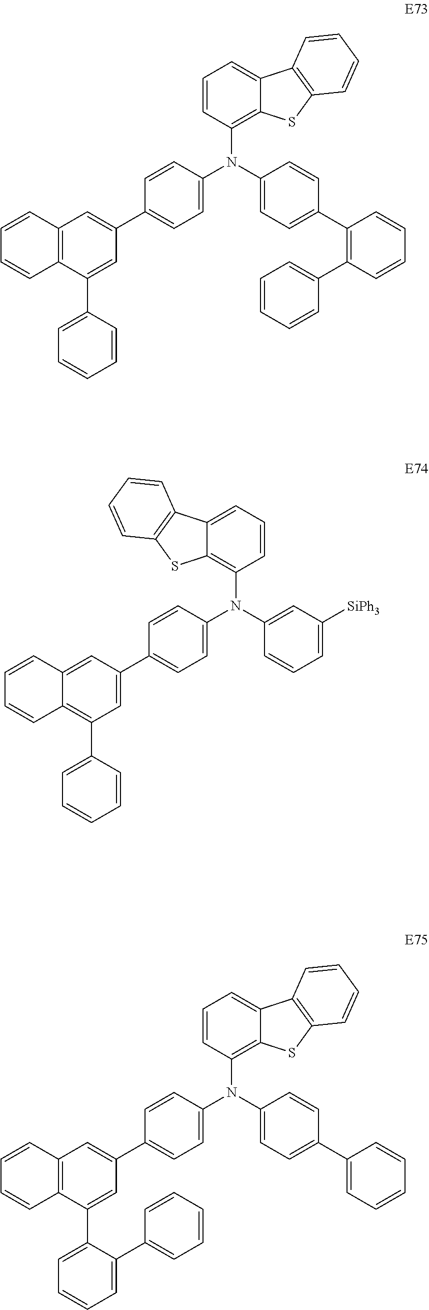

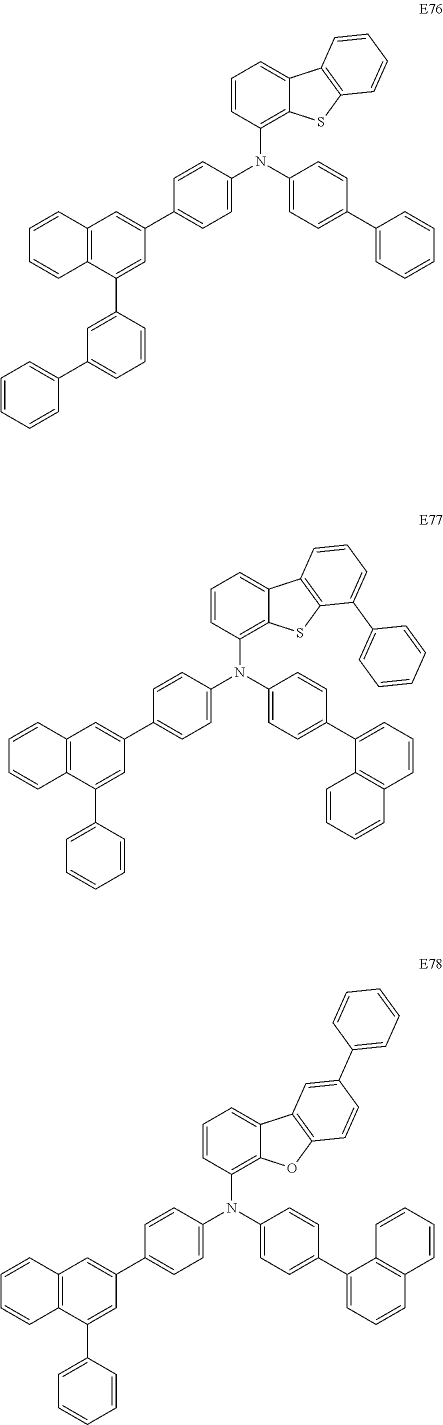 [SMILES: c1ccc(-c2ccc(N(c3ccc(-c4cc(-c5cccc(-c6ccccc6)c5)c5ccccc5c4)cc3)c3cccc4c3sc3ccccc34)cc2)cc1 ; c1ccc(-c2cc(-c3ccc(N(c4ccc(-c5cccc6ccccc56)cc4)c4cccc5c4sc4c(-c6ccccc6)cccc45)cc3)cc3ccccc23)cc1 ; c1ccc(-c2ccc3oc4c(N(c5ccc(-c6cc(-c7ccccc7)c7ccccc7c6)cc5)c5ccc(-c6cccc7ccccc67)cc5)cccc4c3c2)cc1]

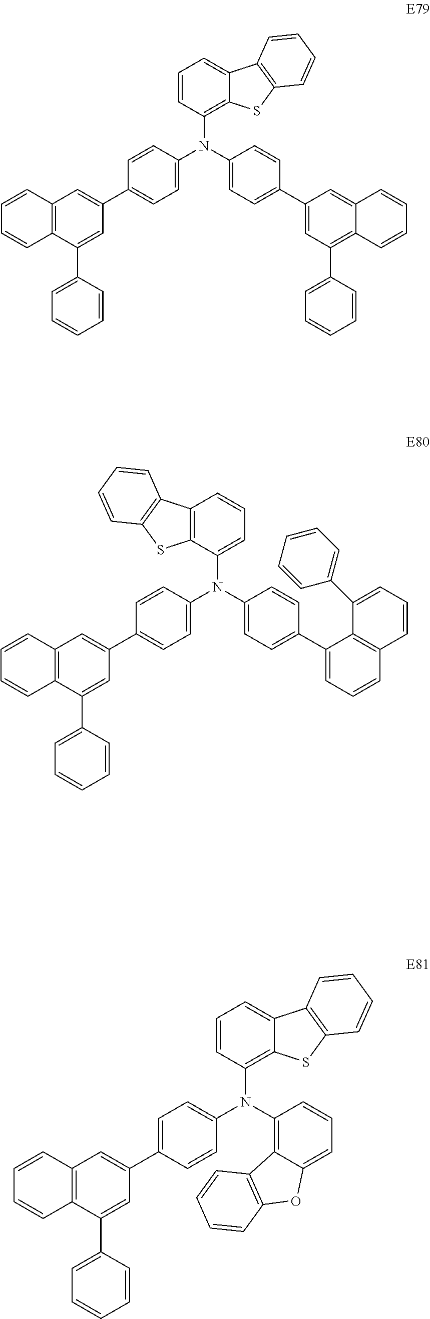 [SMILES: c1ccc(-c2cc(-c3ccc(N(c4ccc(-c5cc(-c6ccccc6)c6ccccc6c5)cc4)c4cccc5c4sc4ccccc45)cc3)cc3ccccc23)cc1 ; c1ccc(-c2cc(-c3ccc(N(c4ccc(-c5cccc6cccc(-c7ccccc7)c56)cc4)c4cccc5c4sc4ccccc45)cc3)cc3ccccc23)cc1 ; c1ccc(-c2cc(-c3ccc(N(c4cccc5c4sc4ccccc45)c4cccc5oc6ccccc6c45)cc3)cc3ccccc23)cc1]

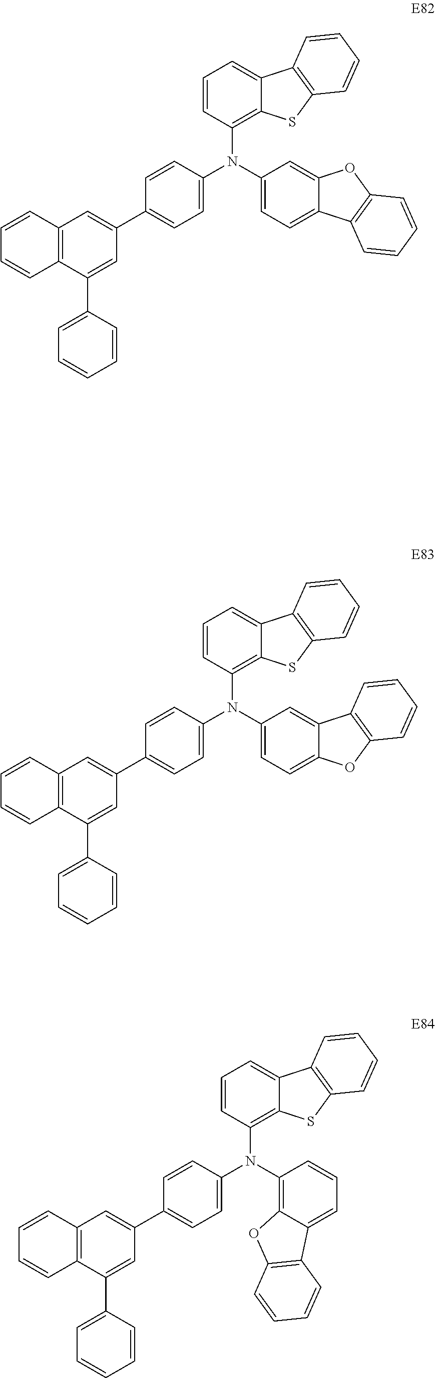 [SMILES: c1ccc(-c2cc(-c3ccc(N(c4ccc5c(c4)oc4ccccc45)c4cccc5c4sc4ccccc45)cc3)cc3ccccc23)cc1 ; c1ccc(-c2cc(-c3ccc(N(c4ccc5oc6ccccc6c5c4)c4cccc5c4sc4ccccc45)cc3)cc3ccccc23)cc1 ; c1ccc(-c2cc(-c3ccc(N(c4cccc5c4oc4ccccc45)c4cccc5c4sc4ccccc45)cc3)cc3ccccc23)cc1]

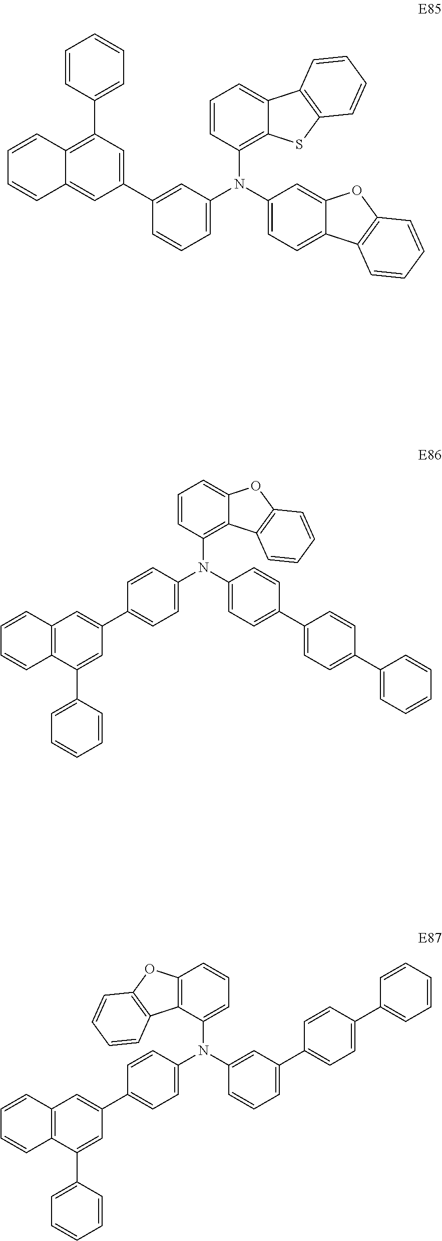 [SMILES: c1ccc(-c2cc(-c3cccc(N(c4ccc5c(c4)oc4ccccc45)c4cccc5c4sc4ccccc45)c3)cc3ccccc23)cc1 ; c1ccc(-c2ccc(-c3ccc(N(c4ccc(-c5cc(-c6ccccc6)c6ccccc6c5)cc4)c4cccc5oc6ccccc6c45)cc3)cc2)cc1 ; c1ccc(-c2ccc(-c3cccc(N(c4ccc(-c5cc(-c6ccccc6)c6ccccc6c5)cc4)c4cccc5oc6ccccc6c45)c3)cc2)cc1]

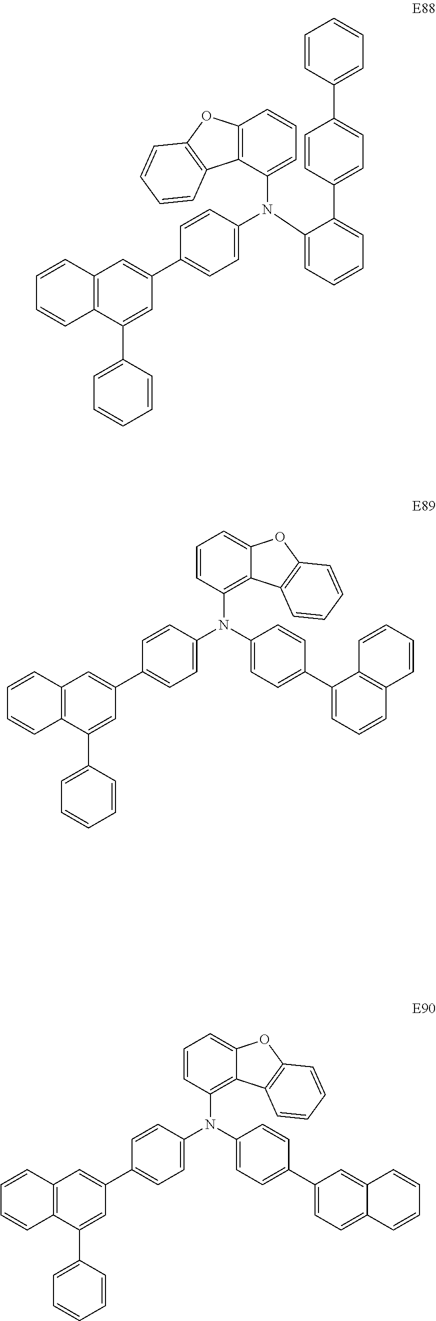 [SMILES: c1ccc(-c2ccc(-c3ccccc3N(c3ccc(-c4cc(-c5ccccc5)c5ccccc5c4)cc3)c3cccc4oc5ccccc5c34)cc2)cc1 ; c1ccc(-c2cc(-c3ccc(N(c4ccc(-c5cccc6ccccc56)cc4)c4cccc5oc6ccccc6c45)cc3)cc3ccccc23)cc1 ; c1ccc(-c2cc(-c3ccc(N(c4ccc(-c5ccc6ccccc6c5)cc4)c4cccc5oc6ccccc6c45)cc3)cc3ccccc23)cc1]

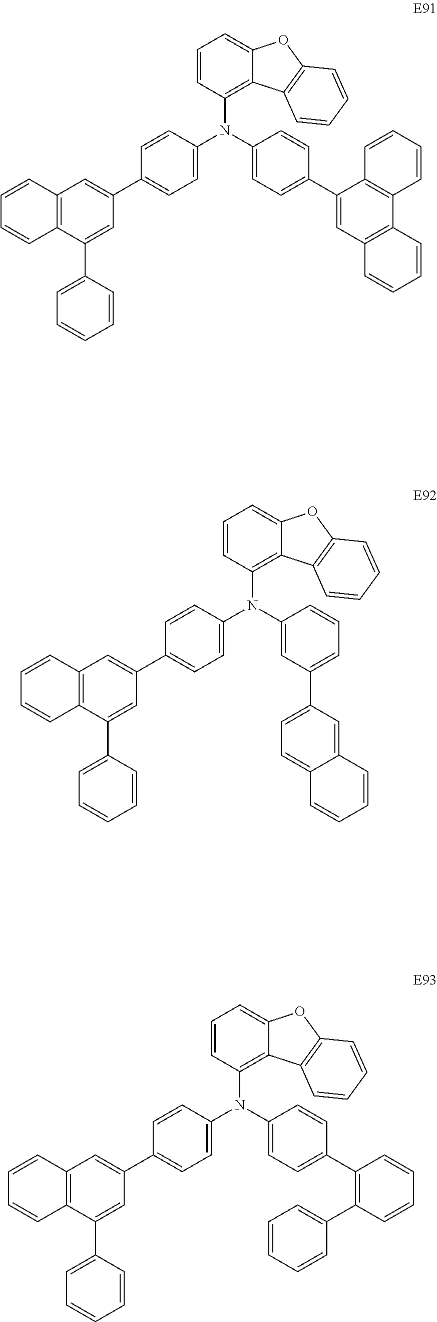 [SMILES: c1ccc(-c2cc(-c3ccc(N(c4ccc(-c5cc6ccccc6c6ccccc56)cc4)c4cccc5oc6ccccc6c45)cc3)cc3ccccc23)cc1 ; c1ccc(-c2cc(-c3ccc(N(c4cccc(-c5ccc6ccccc6c5)c4)c4cccc5oc6ccccc6c45)cc3)cc3ccccc23)cc1 ; c1ccc(-c2ccccc2-c2ccc(N(c3ccc(-c4cc(-c5ccccc5)c5ccccc5c4)cc3)c3cccc4oc5ccccc5c34)cc2)cc1]

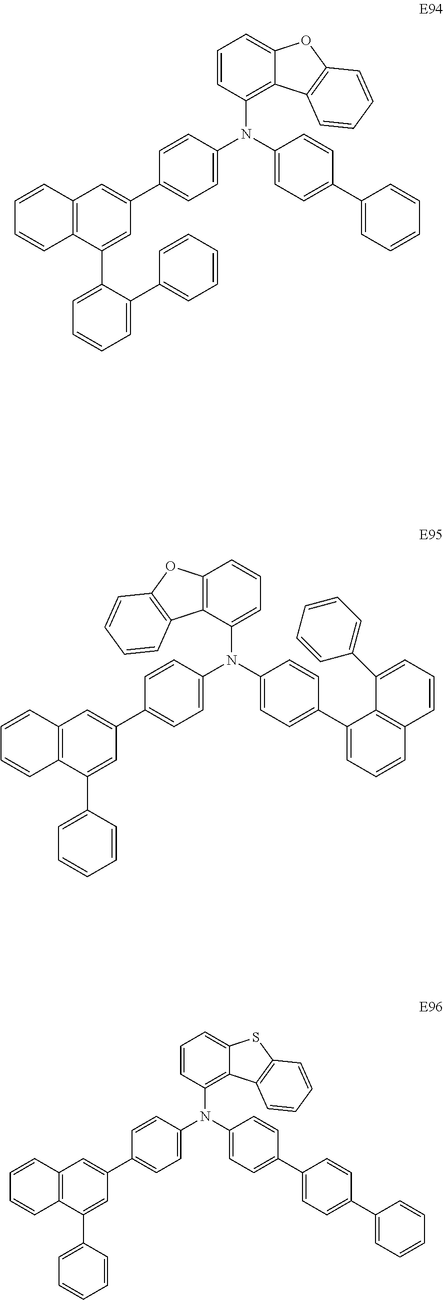 [SMILES: c1ccc(-c2ccc(N(c3ccc(-c4cc(-c5ccccc5-c5ccccc5)c5ccccc5c4)cc3)c3cccc4oc5ccccc5c34)cc2)cc1 ; c1ccc(-c2cc(-c3ccc(N(c4ccc(-c5cccc6cccc(-c7ccccc7)c56)cc4)c4cccc5oc6ccccc6c45)cc3)cc3ccccc23)cc1 ; c1ccc(-c2ccc(-c3ccc(N(c4ccc(-c5cc(-c6ccccc6)c6ccccc6c5)cc4)c4cccc5sc6ccccc6c45)cc3)cc2)cc1]

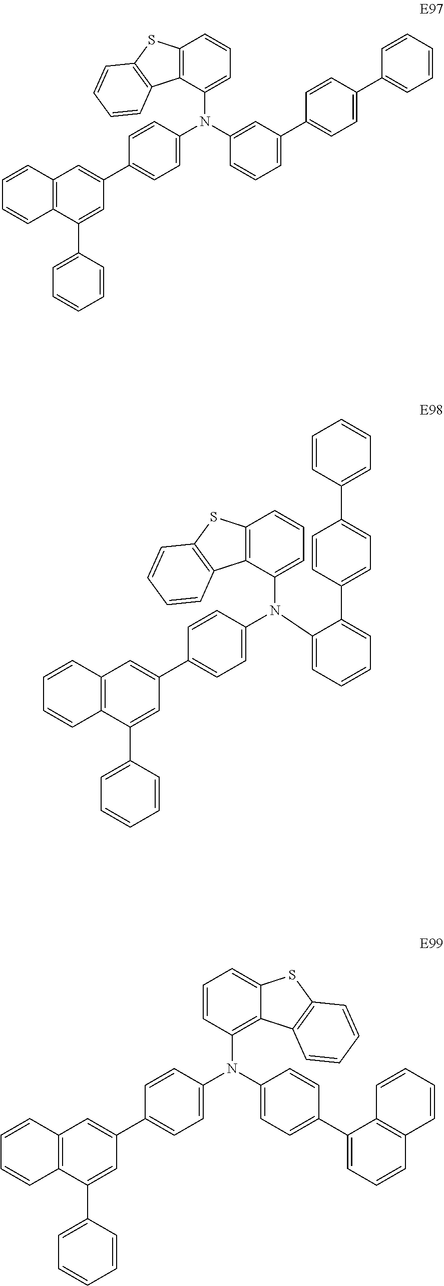 [SMILES: c1ccc(-c2ccc(-c3cccc(N(c4ccc(-c5cc(-c6ccccc6)c6ccccc6c5)cc4)c4cccc5sc6ccccc6c45)c3)cc2)cc1 ; c1ccc(-c2ccc(-c3ccccc3N(c3ccc(-c4cc(-c5ccccc5)c5ccccc5c4)cc3)c3cccc4sc5ccccc5c34)cc2)cc1 ; c1ccc(-c2cc(-c3ccc(N(c4ccc(-c5cccc6ccccc56)cc4)c4cccc5sc6ccccc6c45)cc3)cc3ccccc23)cc1]

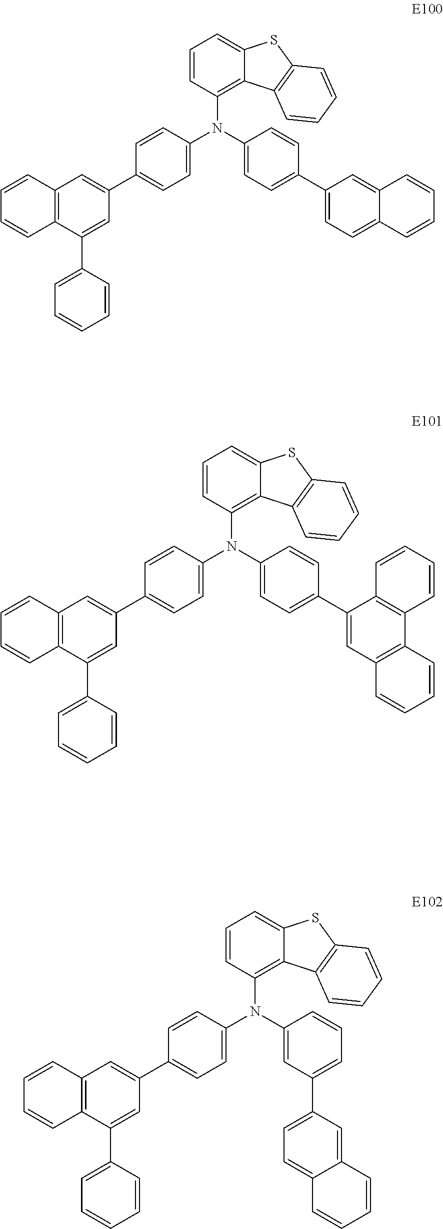 [SMILES: c1ccc(-c2cc(-c3ccc(N(c4ccc(-c5ccc6ccccc6c5)cc4)c4cccc5sc6ccccc6c45)cc3)cc3ccccc23)cc1 ; c1ccc(-c2cc(-c3ccc(N(c4ccc(-c5cc6ccccc6c6ccccc56)cc4)c4cccc5sc6ccccc6c45)cc3)cc3ccccc23)cc1 ; c1ccc(-c2cc(-c3ccc(N(c4cccc(-c5ccc6ccccc6c5)c4)c4cccc5sc6ccccc6c45)cc3)cc3ccccc23)cc1]

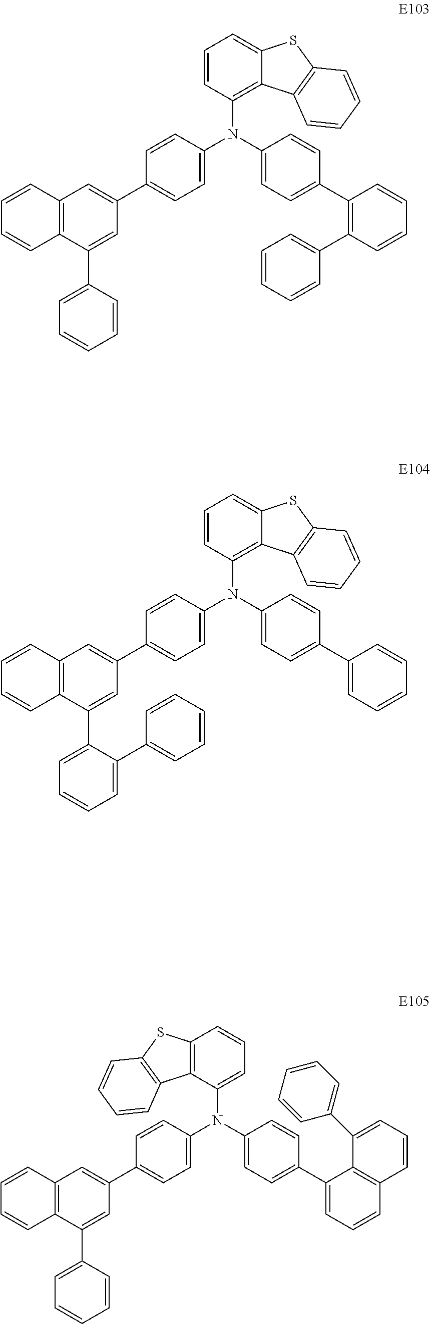 [SMILES: c1ccc(-c2ccccc2-c2ccc(N(c3ccc(-c4cc(-c5ccccc5)c5ccccc5c4)cc3)c3cccc4sc5ccccc5c34)cc2)cc1 ; c1ccc(-c2ccc(N(c3ccc(-c4cc(-c5ccccc5-c5ccccc5)c5ccccc5c4)cc3)c3cccc4sc5ccccc5c34)cc2)cc1 ; c1ccc(-c2cc(-c3ccc(N(c4ccc(-c5cccc6cccc(-c7ccccc7)c56)cc4)c4cccc5sc6ccccc6c45)cc3)cc3ccccc23)cc1]

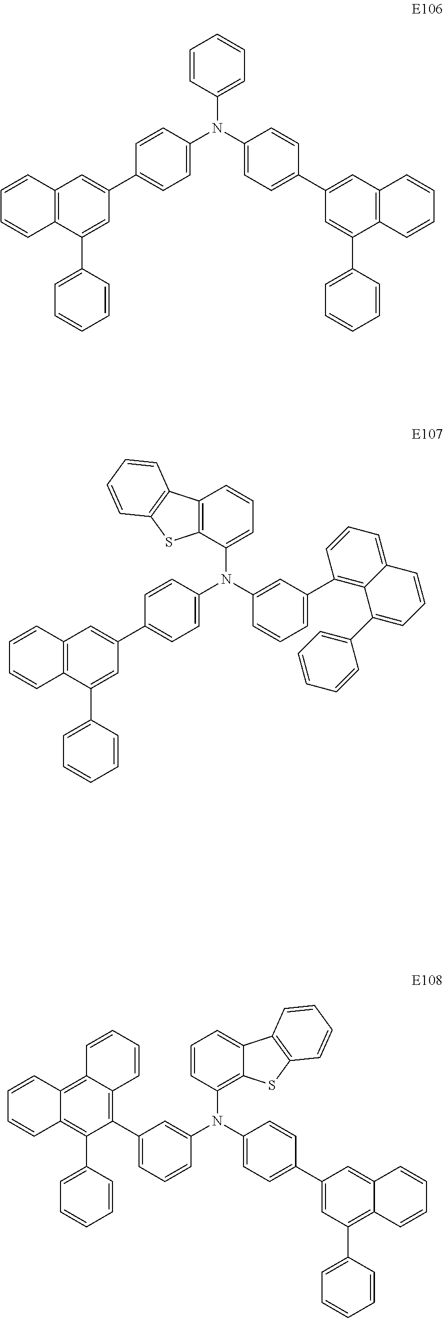 [SMILES: c1ccc(-c2cc(-c3ccc(N(c4ccccc4)c4ccc(-c5cc(-c6ccccc6)c6ccccc6c5)cc4)cc3)cc3ccccc23)cc1 ; c1ccc(-c2cc(-c3ccc(N(c4cccc(-c5cccc6cccc(-c7ccccc7)c56)c4)c4cccc5c4sc4ccccc45)cc3)cc3ccccc23)cc1 ; c1ccc(-c2cc(-c3ccc(N(c4cccc(-c5c(-c6ccccc6)c6ccccc6c6ccccc56)c4)c4cccc5c4sc4ccccc45)cc3)cc3ccccc23)cc1]

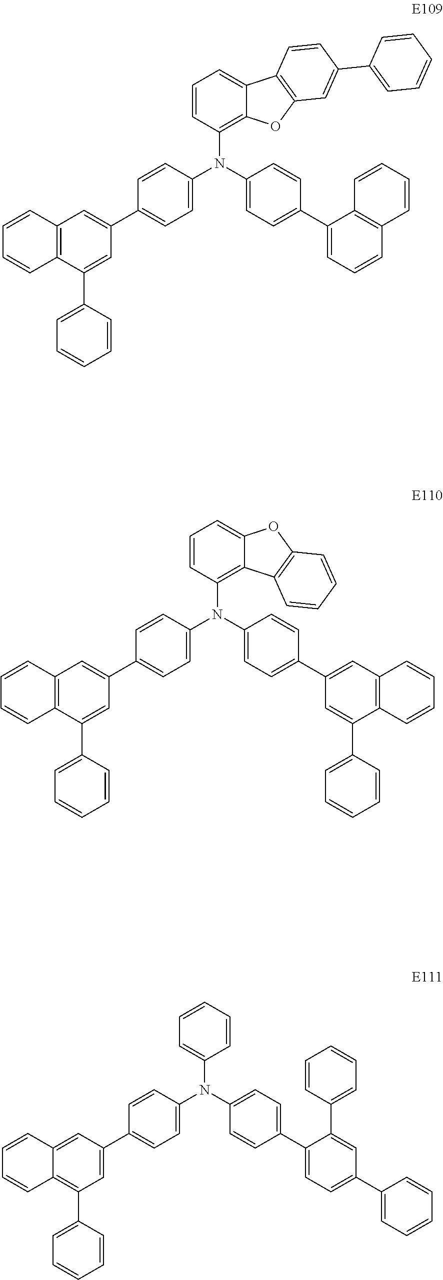 [SMILES: c1ccc(-c2ccc3c(c2)oc2c(N(c4ccc(-c5cc(-c6ccccc6)c6ccccc6c5)cc4)c4ccc(-c5cccc6ccccc56)cc4)cccc23)cc1 ; c1ccc(-c2cc(-c3ccc(N(c4ccc(-c5cc(-c6ccccc6)c6ccccc6c5)cc4)c4cccc5oc6ccccc6c45)cc3)cc3ccccc23)cc1 ; c1ccc(-c2ccc(-c3ccc(N(c4ccccc4)c4ccc(-c5cc(-c6ccccc6)c6ccccc6c5)cc4)cc3)c(-c3ccccc3)c2)cc1]

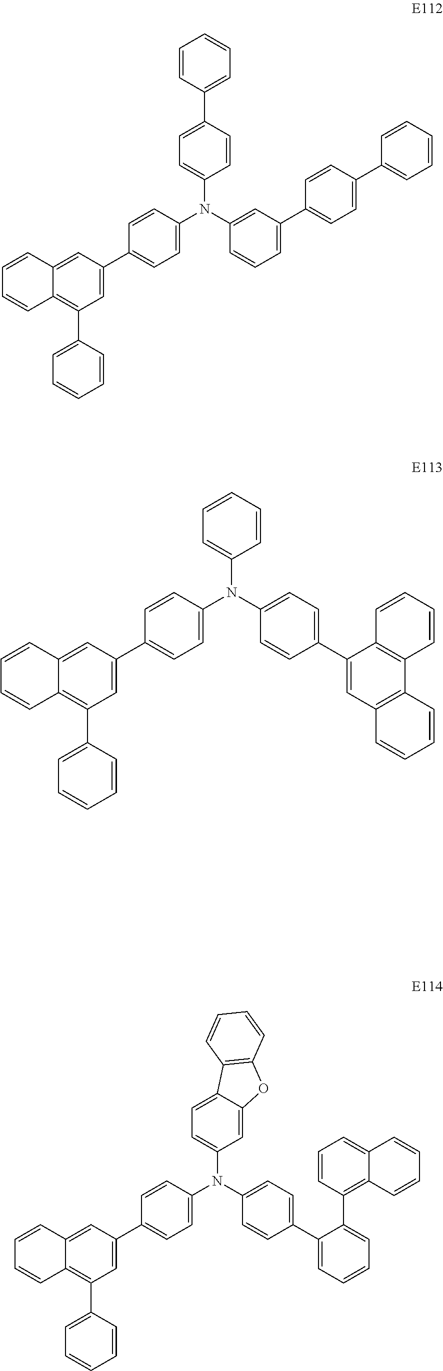 [SMILES: c1ccc(-c2ccc(-c3cccc(N(c4ccc(-c5ccccc5)cc4)c4ccc(-c5cc(-c6ccccc6)c6ccccc6c5)cc4)c3)cc2)cc1 ; c1ccc(-c2cc(-c3ccc(N(c4ccccc4)c4ccc(-c5cc6ccccc6c6ccccc56)cc4)cc3)cc3ccccc23)cc1 ; c1ccc(-c2cc(-c3ccc(N(c4ccc(-c5ccccc5-c5cccc6ccccc56)cc4)c4ccc5c(c4)oc4ccccc45)cc3)cc3ccccc23)cc1]

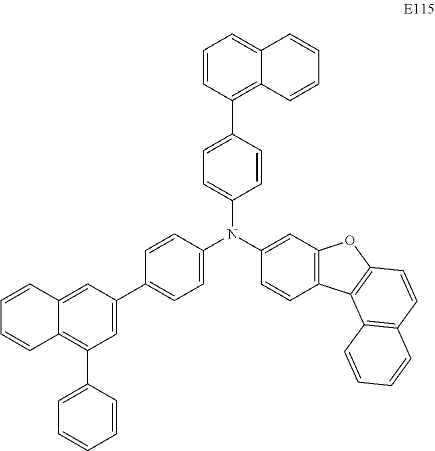 [SMILES: c1ccc(-c2cc(-c3ccc(N(c4ccc(-c5cccc6ccccc56)cc4)c4ccc5c(c4)oc4ccc6ccccc6c45)cc3)cc3ccccc23)cc1]

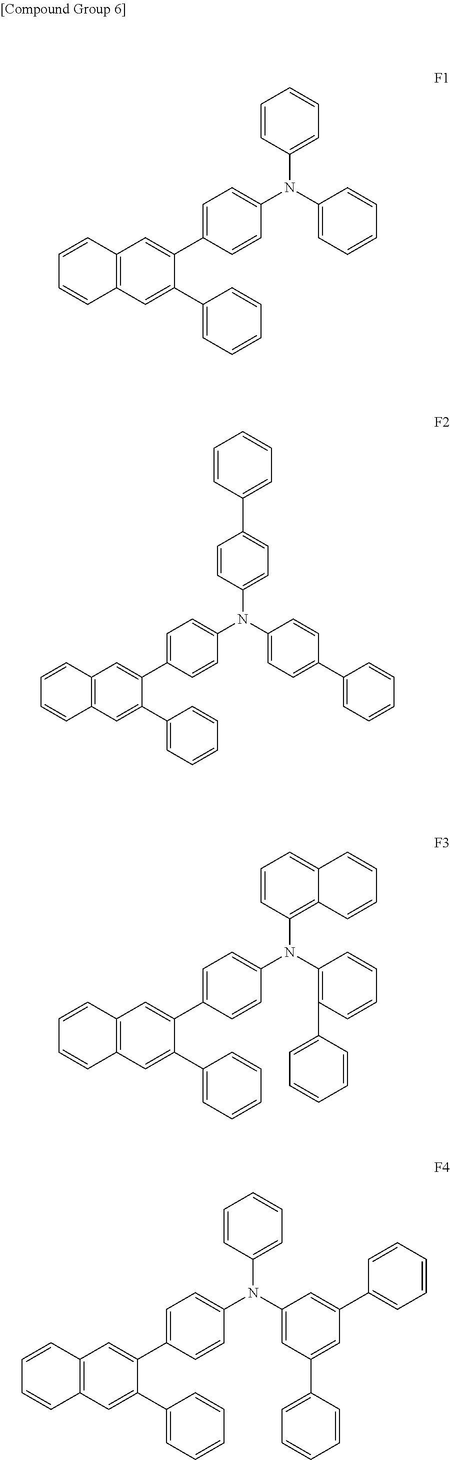 [SMILES: c1ccc(-c2cc3ccccc3cc2-c2ccc(N(c3ccccc3)c3ccccc3)cc2)cc1 ; c1ccc(-c2ccc(N(c3ccc(-c4ccccc4)cc3)c3ccc(-c4cc5ccccc5cc4-c4ccccc4)cc3)cc2)cc1 ; c1ccc(-c2cc3ccccc3cc2-c2ccc(N(c3ccccc3-c3ccccc3)c3cccc4ccccc34)cc2)cc1 ; c1ccc(-c2cc(-c3ccccc3)cc(N(c3ccccc3)c3ccc(-c4cc5ccccc5cc4-c4ccccc4)cc3)c2)cc1]

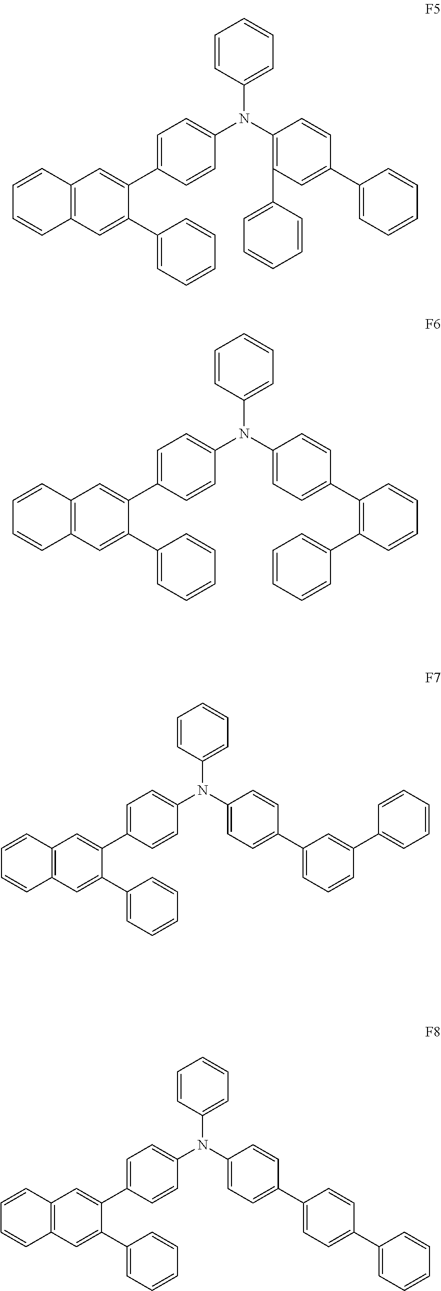 [SMILES: c1ccc(-c2ccc(N(c3ccccc3)c3ccc(-c4cc5ccccc5cc4-c4ccccc4)cc3)c(-c3ccccc3)c2)cc1 ; c1ccc(-c2ccccc2-c2ccc(N(c3ccccc3)c3ccc(-c4cc5ccccc5cc4-c4ccccc4)cc3)cc2)cc1 ; c1ccc(-c2cccc(-c3ccc(N(c4ccccc4)c4ccc(-c5cc6ccccc6cc5-c5ccccc5)cc4)cc3)c2)cc1 ; c1ccc(-c2ccc(-c3ccc(N(c4ccccc4)c4ccc(-c5cc6ccccc6cc5-c5ccccc5)cc4)cc3)cc2)cc1]

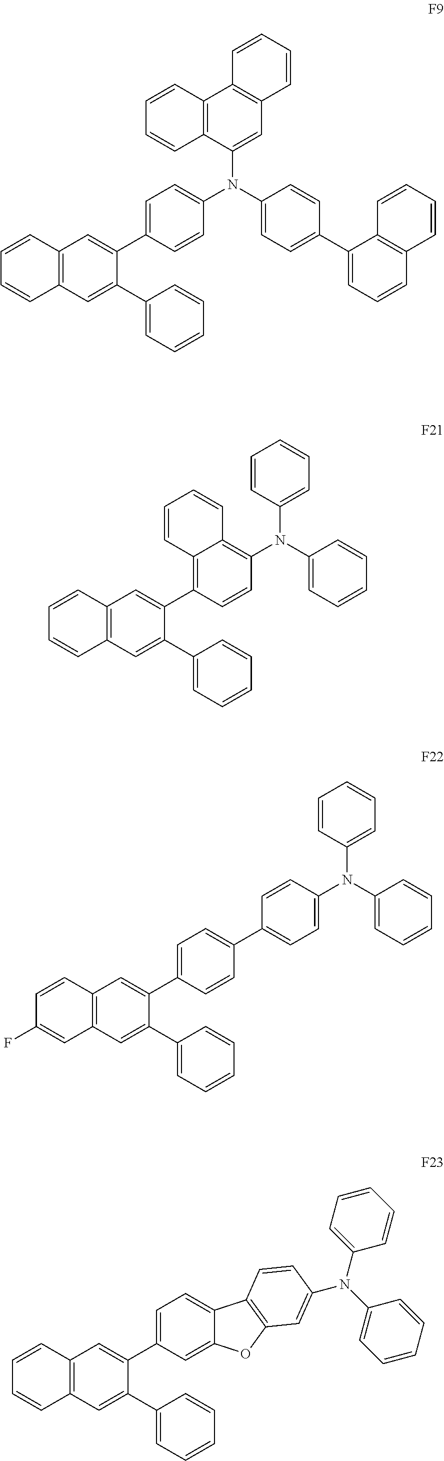 [SMILES: c1ccc(-c2cc3ccccc3cc2-c2ccc(N(c3ccc(-c4cccc5ccccc45)cc3)c3cc4ccccc4c4ccccc34)cc2)cc1 ; c1ccc(-c2cc3ccccc3cc2-c2ccc(N(c3ccccc3)c3ccccc3)c3ccccc23)cc1 ; Fc1ccc2cc(-c3ccc(-c4ccc(N(c5ccccc5)c5ccccc5)cc4)cc3)c(-c3ccccc3)cc2c1 ; c1ccc(-c2cc3ccccc3cc2-c2ccc3c(c2)oc2cc(N(c4ccccc4)c4ccccc4)ccc23)cc1]

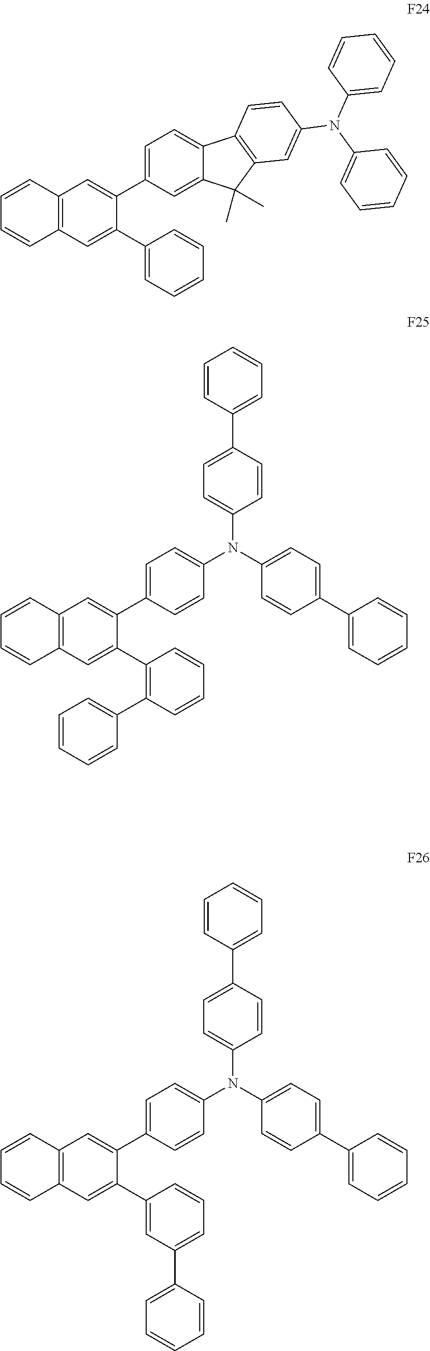 [SMILES: CC1(C)c2cc(-c3cc4ccccc4cc3-c3ccccc3)ccc2-c2ccc(N(c3ccccc3)c3ccccc3)cc21 ; c1ccc(-c2ccc(N(c3ccc(-c4ccccc4)cc3)c3ccc(-c4cc5ccccc5cc4-c4ccccc4-c4ccccc4)cc3)cc2)cc1 ; c1ccc(-c2ccc(N(c3ccc(-c4ccccc4)cc3)c3ccc(-c4cc5ccccc5cc4-c4cccc(-c5ccccc5)c4)cc3)cc2)cc1]

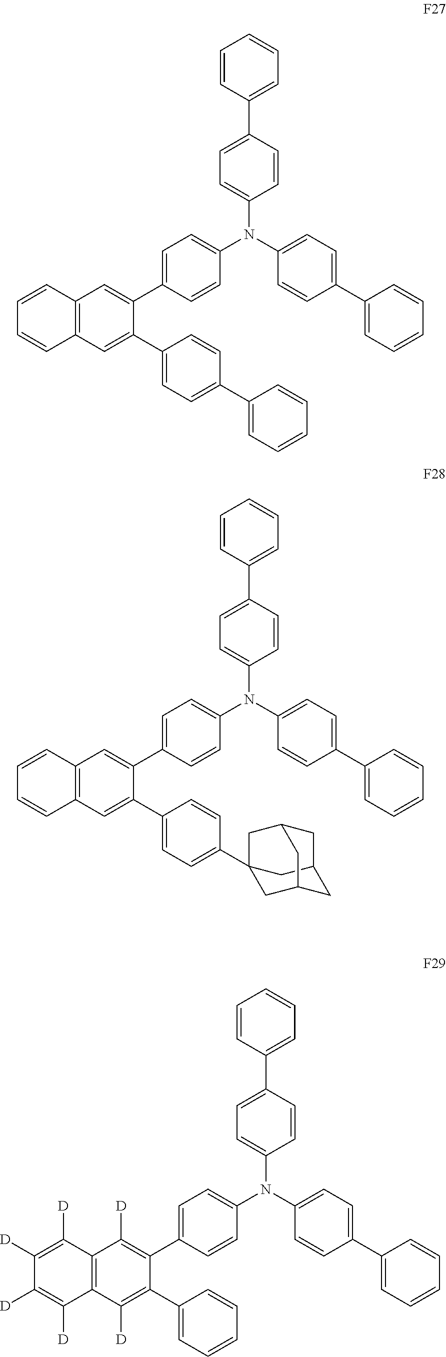 [SMILES: c1ccc(-c2ccc(-c3cc4ccccc4cc3-c3ccc(N(c4ccc(-c5ccccc5)cc4)c4ccc(-c5ccccc5)cc4)cc3)cc2)cc1 ; c1ccc(-c2ccc(N(c3ccc(-c4ccccc4)cc3)c3ccc(-c4cc5ccccc5cc4-c4ccc(C56CC7CC(CC(C7)C5)C6)cc4)cc3)cc2)cc1 ; [2H]c1c([2H])c([2H])c2c([2H])c(-c3ccc(N(c4ccc(-c5ccccc5)cc4)c4ccc(-c5ccccc5)cc4)cc3)c(-c3ccccc3)c([2H])c2c1[2H]]

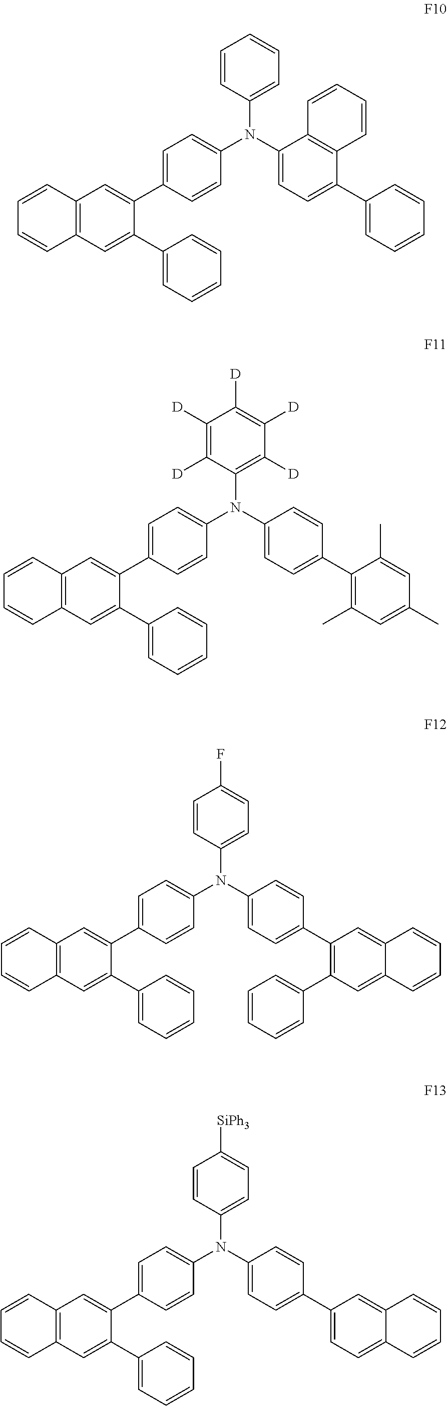

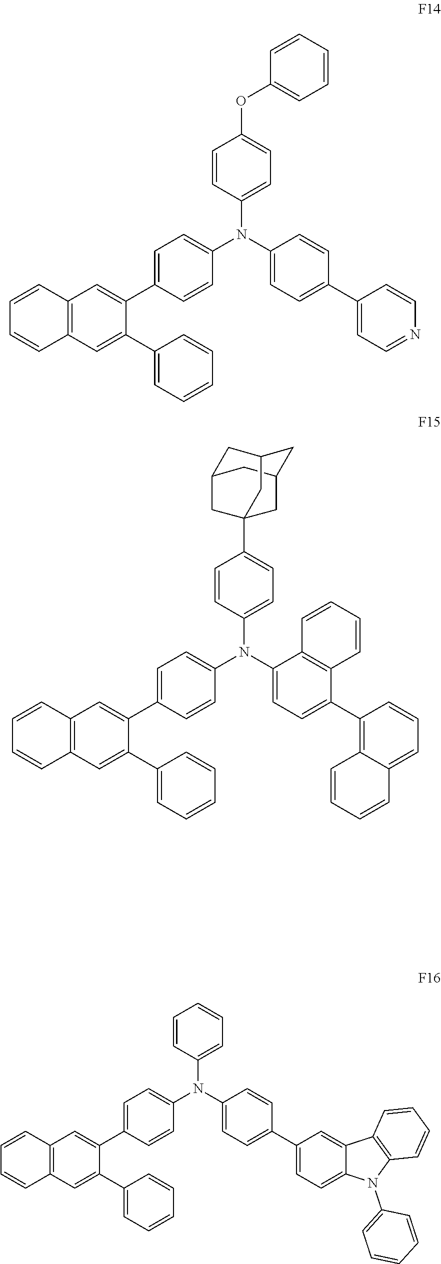 [SMILES: c1ccc(Oc2ccc(N(c3ccc(-c4ccncc4)cc3)c3ccc(-c4cc5ccccc5cc4-c4ccccc4)cc3)cc2)cc1 ; c1ccc(-c2cc3ccccc3cc2-c2ccc(N(c3ccc(C45CC6CC(CC(C6)C4)C5)cc3)c3ccc(-c4cccc5ccccc45)c4ccccc34)cc2)cc1 ; c1ccc(-c2cc3ccccc3cc2-c2ccc(N(c3ccccc3)c3ccc(-c4ccc5c(c4)c4ccccc4n5-c4ccccc4)cc3)cc2)cc1]

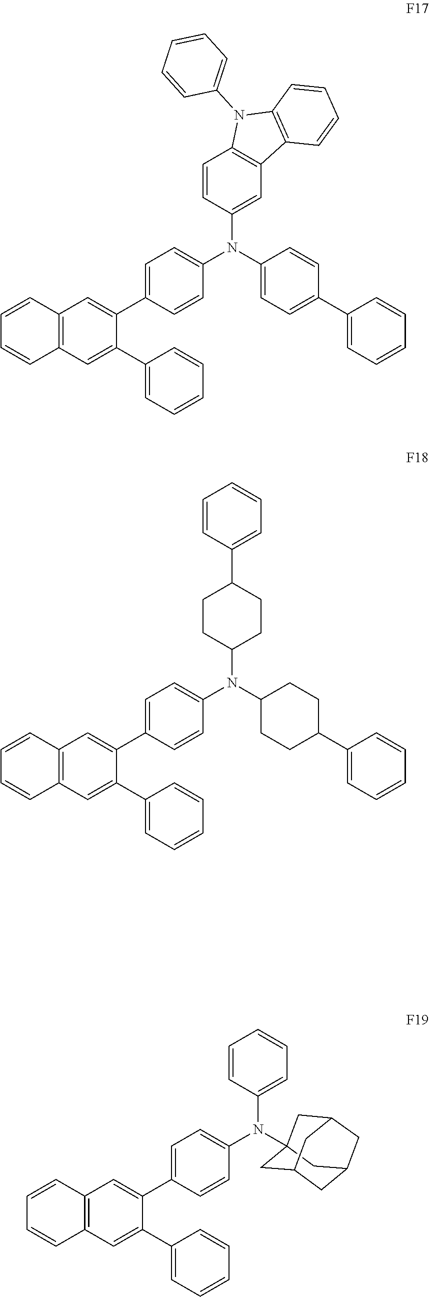 [SMILES: c1ccc(-c2ccc(N(c3ccc(-c4cc5ccccc5cc4-c4ccccc4)cc3)c3ccc4c(c3)c3ccccc3n4-c3ccccc3)cc2)cc1 ; c1ccc(-c2cc3ccccc3cc2-c2ccc(N(C3CCC(c4ccccc4)CC3)C3CCC(c4ccccc4)CC3)cc2)cc1 ; c1ccc(-c2cc3ccccc3cc2-c2ccc(N(c3ccccc3)C34CC5CC(CC(C5)C3)C4)cc2)cc1]

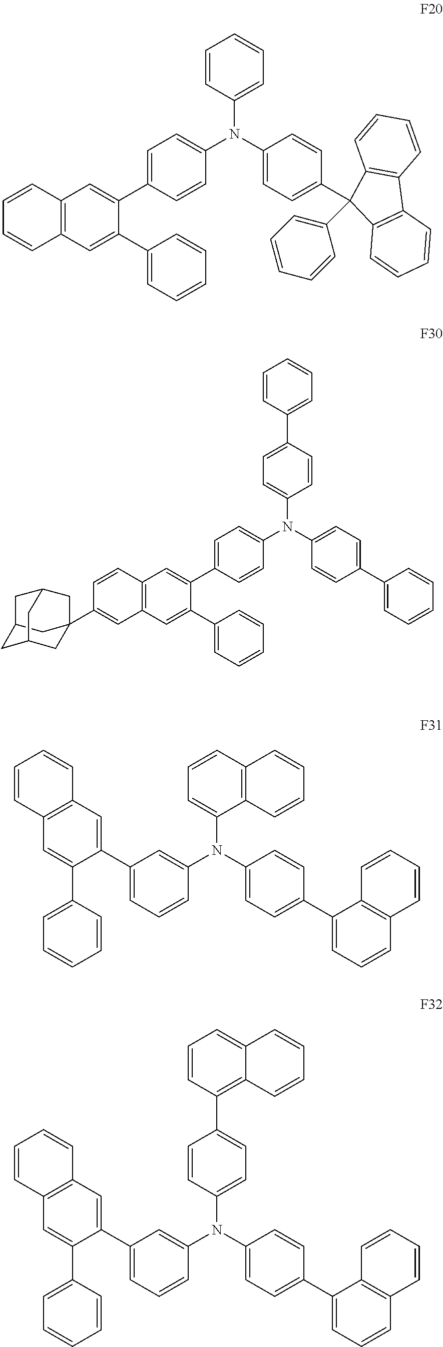 [SMILES: c1ccc(-c2cc3ccccc3cc2-c2ccc(N(c3ccccc3)c3ccc(C4(c5ccccc5)c5ccccc5-c5ccccc54)cc3)cc2)cc1 ; c1ccc(-c2ccc(N(c3ccc(-c4ccccc4)cc3)c3ccc(-c4cc5ccc(C67CC8CC(CC(C8)C6)C7)cc5cc4-c4ccccc4)cc3)cc2)cc1 ; c1ccc(-c2cc3ccccc3cc2-c2cccc(N(c3ccc(-c4cccc5ccccc45)cc3)c3cccc4ccccc34)c2)cc1 ; c1ccc(-c2cc3ccccc3cc2-c2cccc(N(c3ccc(-c4cccc5ccccc45)cc3)c3ccc(-c4cccc5ccccc45)cc3)c2)cc1]

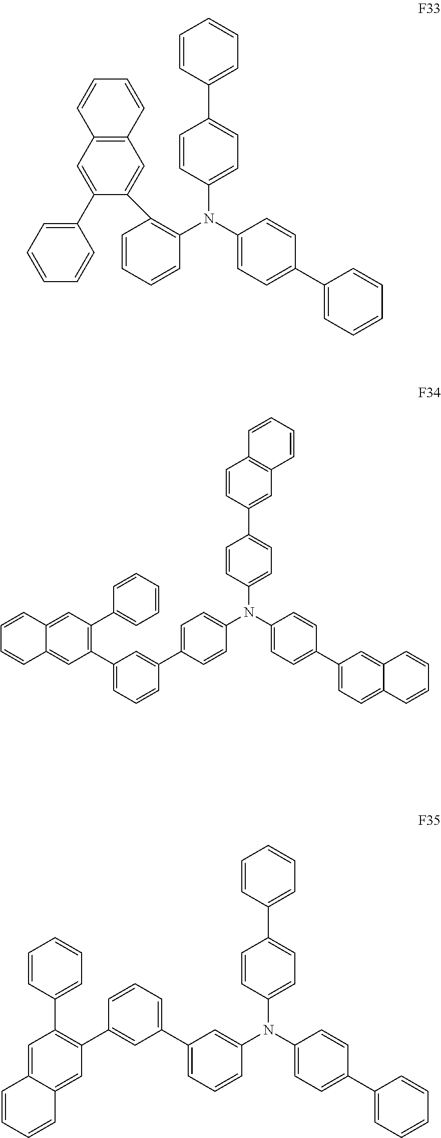 [SMILES: c1ccc(-c2ccc(N(c3ccc(-c4ccccc4)cc3)c3ccccc3-c3cc4ccccc4cc3-c3ccccc3)cc2)cc1 ; c1ccc(-c2cc3ccccc3cc2-c2cccc(-c3ccc(N(c4ccc(-c5ccc6ccccc6c5)cc4)c4ccc(-c5ccc6ccccc6c5)cc4)cc3)c2)cc1 ; c1ccc(-c2ccc(N(c3ccc(-c4ccccc4)cc3)c3cccc(-c4cccc(-c5cc6ccccc6cc5-c5ccccc5)c4)c3)cc2)cc1]

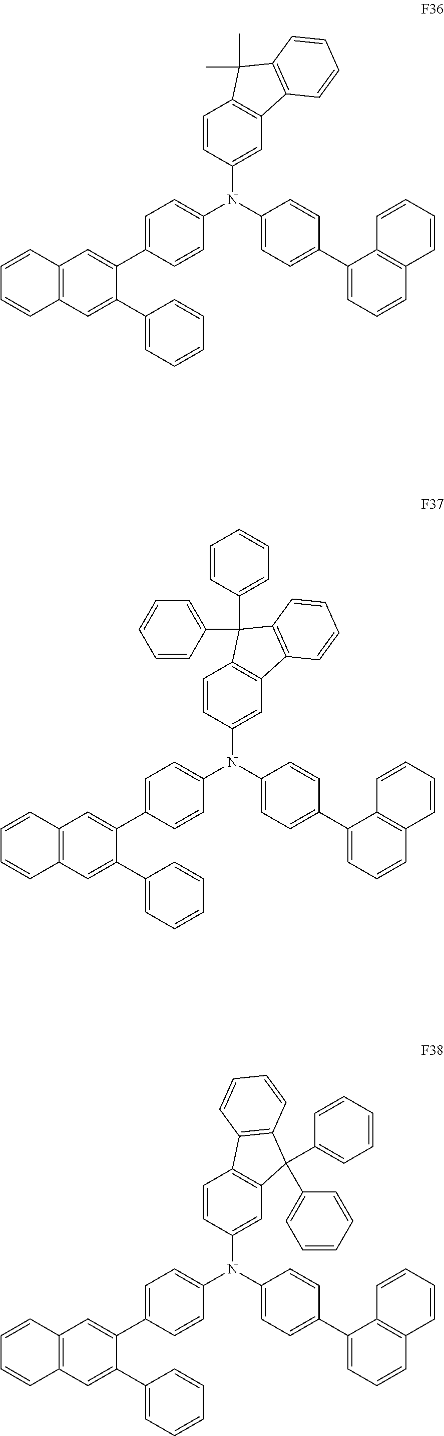 [SMILES: CC1(C)c2ccccc2-c2cc(N(c3ccc(-c4cc5ccccc5cc4-c4ccccc4)cc3)c3ccc(-c4cccc5ccccc45)cc3)ccc21 ; c1ccc(-c2cc3ccccc3cc2-c2ccc(N(c3ccc(-c4cccc5ccccc45)cc3)c3ccc4c(c3)-c3ccccc3C4(c3ccccc3)c3ccccc3)cc2)cc1 ; c1ccc(-c2cc3ccccc3cc2-c2ccc(N(c3ccc(-c4cccc5ccccc45)cc3)c3ccc4c(c3)C(c3ccccc3)(c3ccccc3)c3ccccc3-4)cc2)cc1]

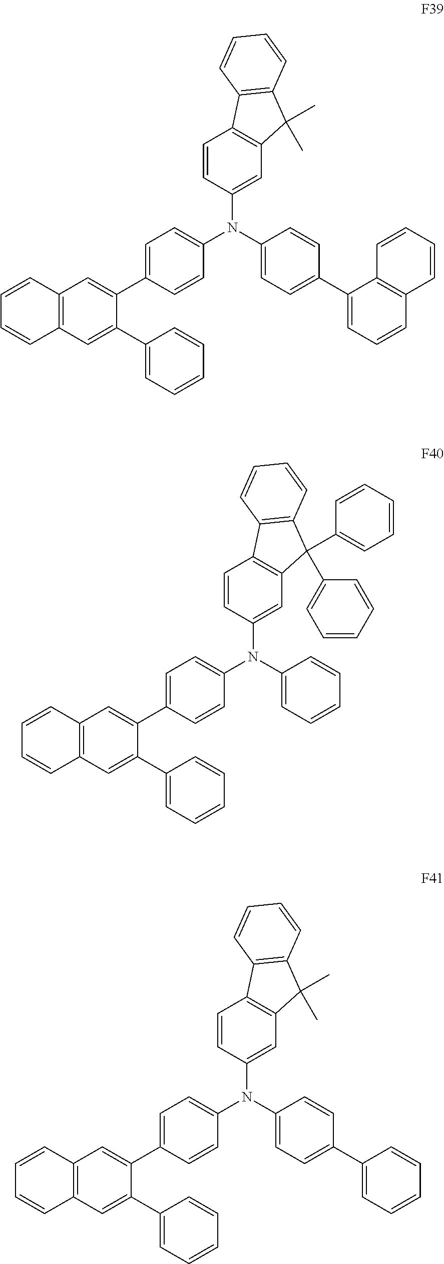 [SMILES: CC1(C)c2ccccc2-c2ccc(N(c3ccc(-c4cc5ccccc5cc4-c4ccccc4)cc3)c3ccc(-c4cccc5ccccc45)cc3)cc21 ; c1ccc(-c2cc3ccccc3cc2-c2ccc(N(c3ccccc3)c3ccc4c(c3)C(c3ccccc3)(c3ccccc3)c3ccccc3-4)cc2)cc1 ; CC1(C)c2ccccc2-c2ccc(N(c3ccc(-c4ccccc4)cc3)c3ccc(-c4cc5ccccc5cc4-c4ccccc4)cc3)cc21]

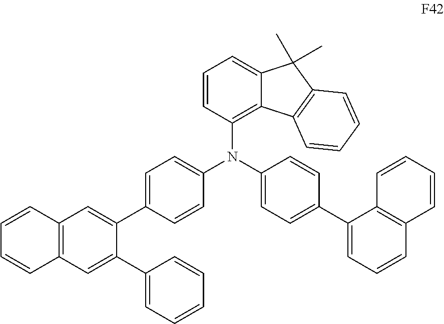 [SMILES: CC1(C)c2ccccc2-c2c(N(c3ccc(-c4cc5ccccc5cc4-c4ccccc4)cc3)c3ccc(-c4cccc5ccccc45)cc3)cccc21]

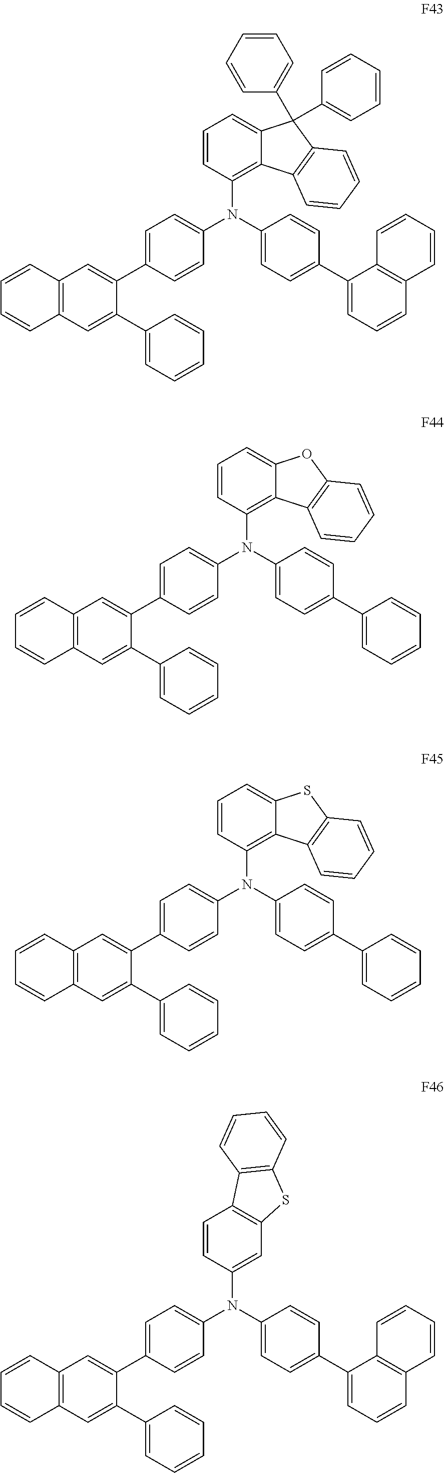 [SMILES: c1ccc(-c2cc3ccccc3cc2-c2ccc(N(c3ccc(-c4cccc5ccccc45)cc3)c3cccc4c3-c3ccccc3C4(c3ccccc3)c3ccccc3)cc2)cc1 ; c1ccc(-c2ccc(N(c3ccc(-c4cc5ccccc5cc4-c4ccccc4)cc3)c3cccc4oc5ccccc5c34)cc2)cc1 ; c1ccc(-c2ccc(N(c3ccc(-c4cc5ccccc5cc4-c4ccccc4)cc3)c3cccc4sc5ccccc5c34)cc2)cc1 ; c1ccc(-c2cc3ccccc3cc2-c2ccc(N(c3ccc(-c4cccc5ccccc45)cc3)c3ccc4c(c3)sc3ccccc34)cc2)cc1]

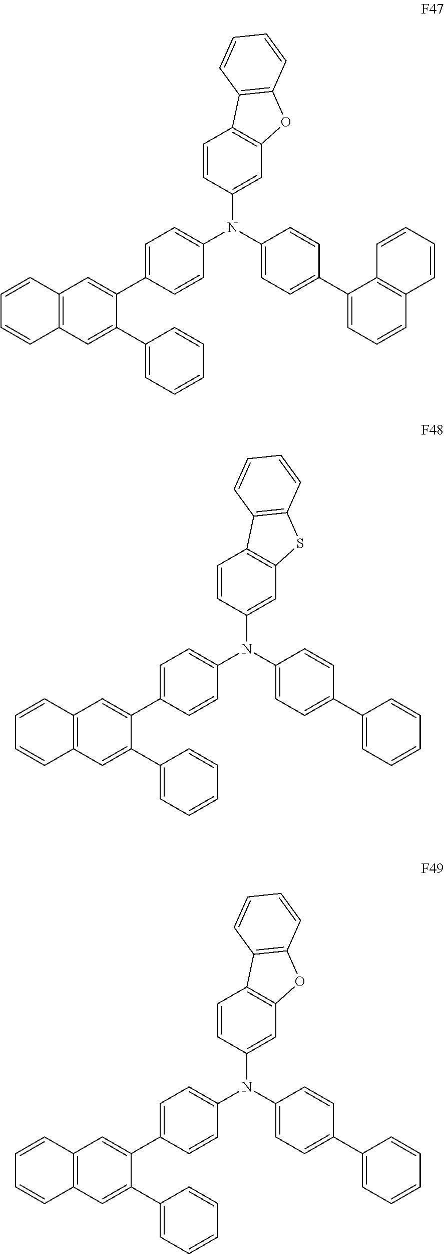 [SMILES: c1ccc(-c2cc3ccccc3cc2-c2ccc(N(c3ccc(-c4cccc5ccccc45)cc3)c3ccc4c(c3)oc3ccccc34)cc2)cc1 ; c1ccc(-c2ccc(N(c3ccc(-c4cc5ccccc5cc4-c4ccccc4)cc3)c3ccc4c(c3)sc3ccccc34)cc2)cc1 ; c1ccc(-c2ccc(N(c3ccc(-c4cc5ccccc5cc4-c4ccccc4)cc3)c3ccc4c(c3)oc3ccccc34)cc2)cc1]

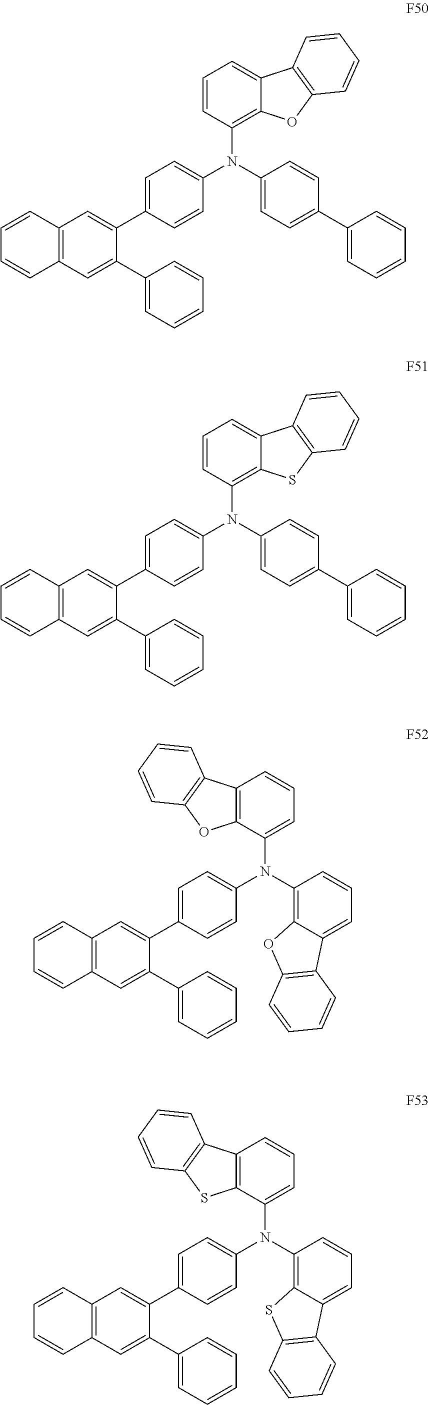 [SMILES: c1ccc(-c2ccc(N(c3ccc(-c4cc5ccccc5cc4-c4ccccc4)cc3)c3cccc4c3oc3ccccc34)cc2)cc1 ; c1ccc(-c2ccc(N(c3ccc(-c4cc5ccccc5cc4-c4ccccc4)cc3)c3cccc4c3sc3ccccc34)cc2)cc1 ; c1ccc(-c2cc3ccccc3cc2-c2ccc(N(c3cccc4c3oc3ccccc34)c3cccc4c3oc3ccccc34)cc2)cc1 ; c1ccc(-c2cc3ccccc3cc2-c2ccc(N(c3cccc4c3sc3ccccc34)c3cccc4c3sc3ccccc34)cc2)cc1]

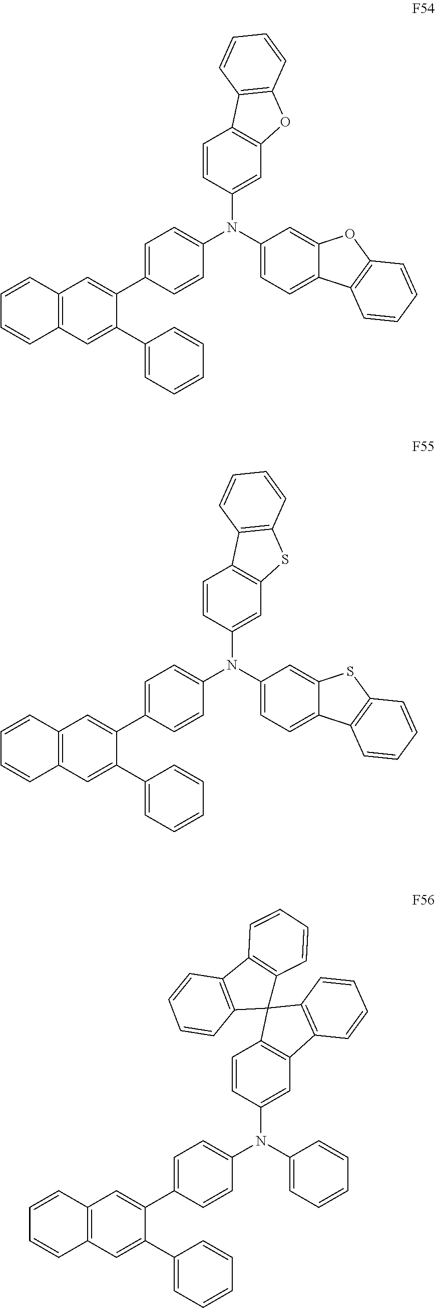 [SMILES: c1ccc(-c2cc3ccccc3cc2-c2ccc(N(c3ccc4c(c3)oc3ccccc34)c3ccc4c(c3)oc3ccccc34)cc2)cc1 ; c1ccc(-c2cc3ccccc3cc2-c2ccc(N(c3ccc4c(c3)sc3ccccc34)c3ccc4c(c3)sc3ccccc34)cc2)cc1 ; c1ccc(-c2cc3ccccc3cc2-c2ccc(N(c3ccccc3)c3ccc4c(c3)-c3ccccc3C43c4ccccc4-c4ccccc43)cc2)cc1]

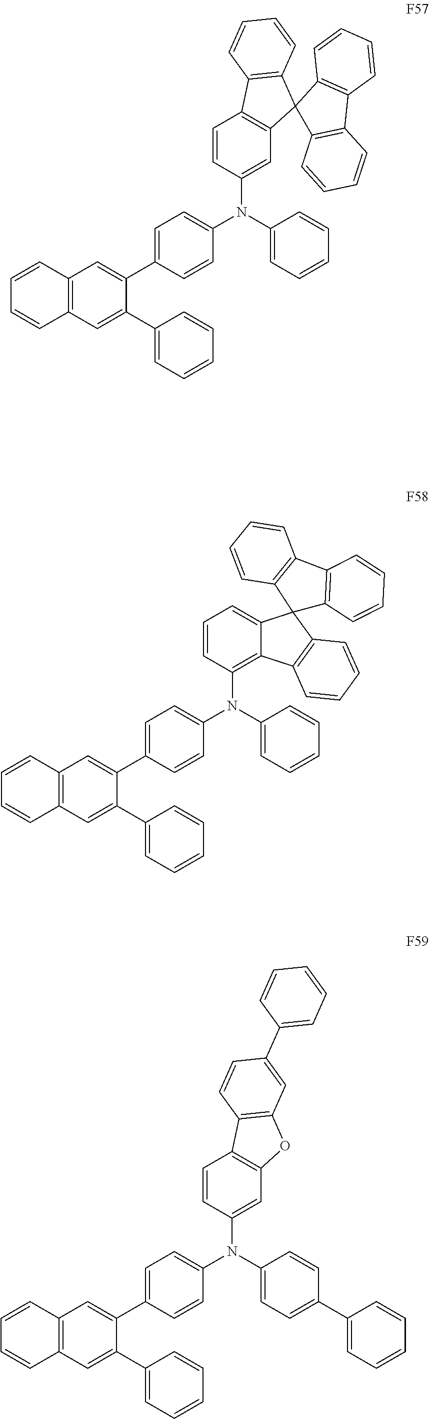 [SMILES: c1ccc(-c2cc3ccccc3cc2-c2ccc(N(c3ccccc3)c3ccc4c(c3)C3(c5ccccc5-c5ccccc53)c3ccccc3-4)cc2)cc1 ; c1ccc(-c2cc3ccccc3cc2-c2ccc(N(c3ccccc3)c3cccc4c3-c3ccccc3C43c4ccccc4-c4ccccc43)cc2)cc1 ; c1ccc(-c2ccc(N(c3ccc(-c4cc5ccccc5cc4-c4ccccc4)cc3)c3ccc4c(c3)oc3cc(-c5ccccc5)ccc34)cc2)cc1]

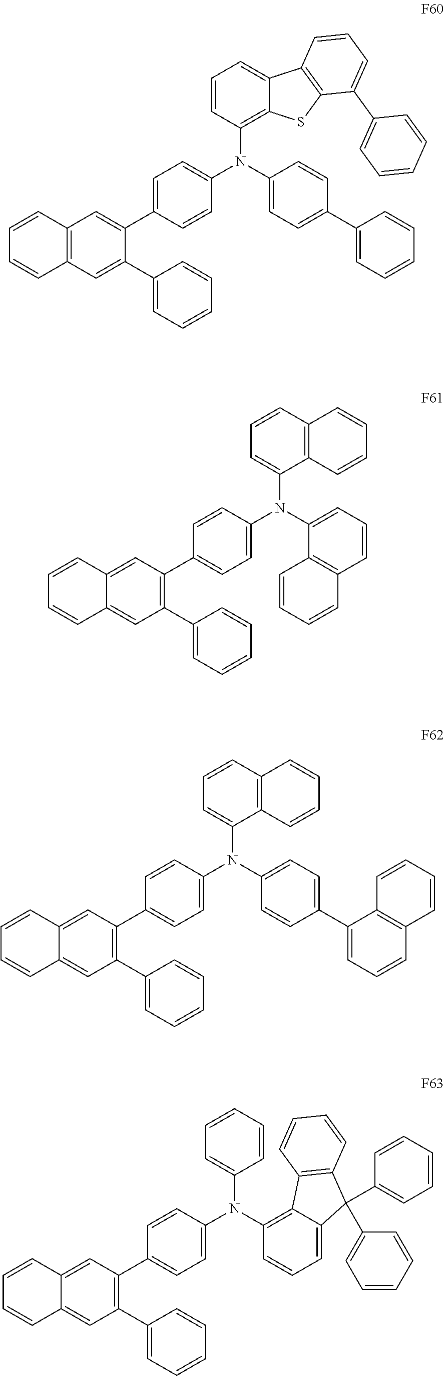 [SMILES: c1ccc(-c2ccc(N(c3ccc(-c4cc5ccccc5cc4-c4ccccc4)cc3)c3cccc4c3sc3c(-c5ccccc5)cccc34)cc2)cc1 ; c1ccc(-c2cc3ccccc3cc2-c2ccc(N(c3cccc4ccccc34)c3cccc4ccccc34)cc2)cc1 ; c1ccc(-c2cc3ccccc3cc2-c2ccc(N(c3ccc(-c4cccc5ccccc45)cc3)c3cccc4ccccc34)cc2)cc1 ; c1ccc(-c2cc3ccccc3cc2-c2ccc(N(c3ccccc3)c3cccc4c3-c3ccccc3C4(c3ccccc3)c3ccccc3)cc2)cc1]

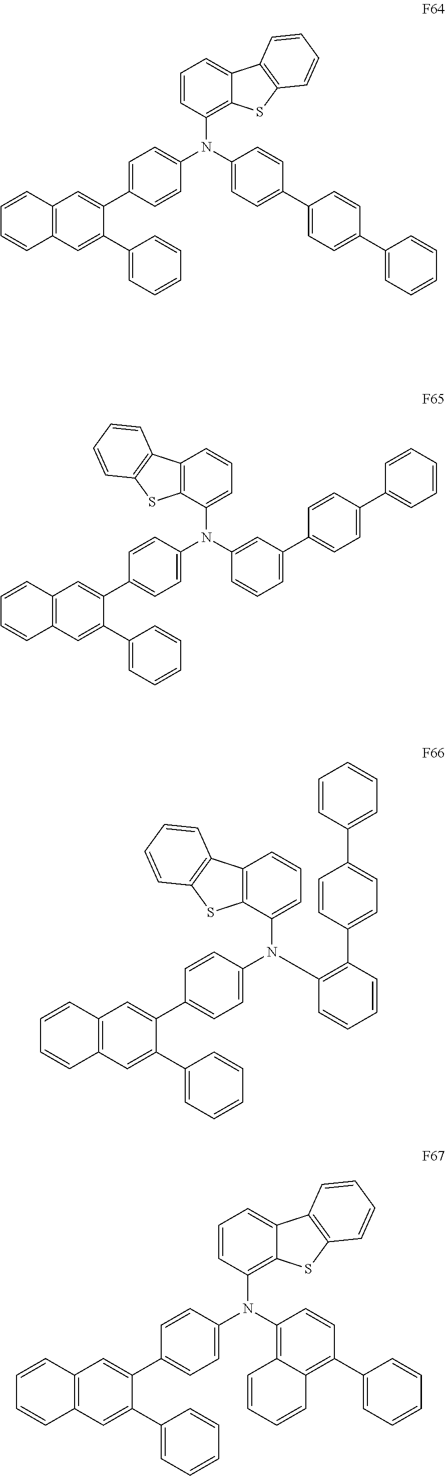 [SMILES: c1ccc(-c2ccc(-c3ccc(N(c4ccc(-c5cc6ccccc6cc5-c5ccccc5)cc4)c4cccc5c4sc4ccccc45)cc3)cc2)cc1 ; c1ccc(-c2ccc(-c3cccc(N(c4ccc(-c5cc6ccccc6cc5-c5ccccc5)cc4)c4cccc5c4sc4ccccc45)c3)cc2)cc1 ; c1ccc(-c2ccc(-c3ccccc3N(c3ccc(-c4cc5ccccc5cc4-c4ccccc4)cc3)c3cccc4c3sc3ccccc34)cc2)cc1 ; c1ccc(-c2cc3ccccc3cc2-c2ccc(N(c3ccc(-c4ccccc4)c4ccccc34)c3cccc4c3sc3ccccc34)cc2)cc1]

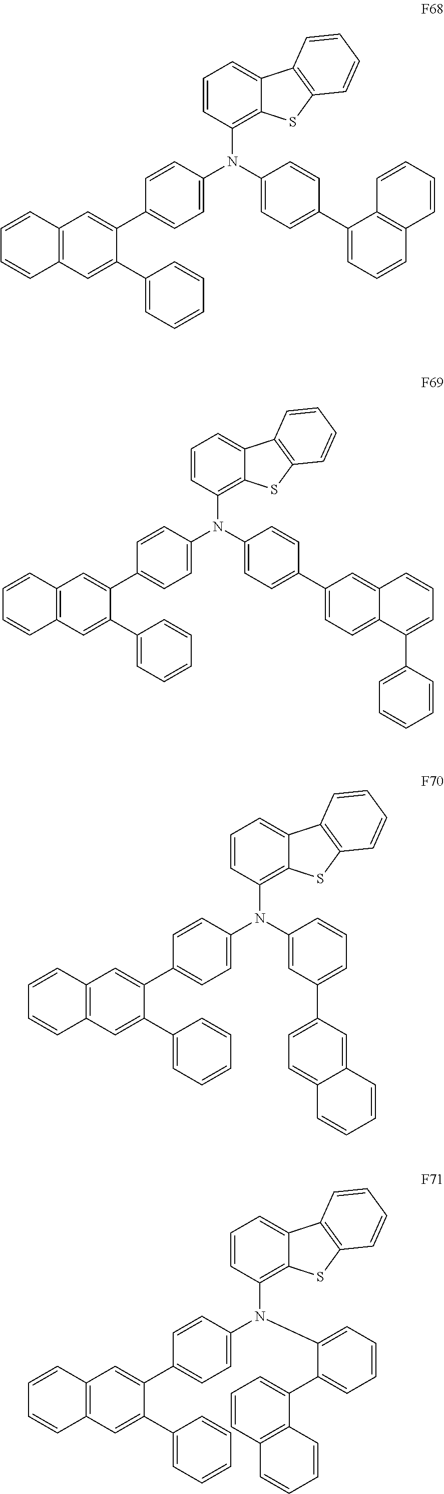 [SMILES: c1ccc(-c2cc3ccccc3cc2-c2ccc(N(c3ccc(-c4cccc5ccccc45)cc3)c3cccc4c3sc3ccccc34)cc2)cc1 ; c1ccc(-c2cc3ccccc3cc2-c2ccc(N(c3ccc(-c4ccc5c(-c6ccccc6)cccc5c4)cc3)c3cccc4c3sc3ccccc34)cc2)cc1 ; c1ccc(-c2cc3ccccc3cc2-c2ccc(N(c3cccc(-c4ccc5ccccc5c4)c3)c3cccc4c3sc3ccccc34)cc2)cc1 ; c1ccc(-c2cc3ccccc3cc2-c2ccc(N(c3ccccc3-c3cccc4ccccc34)c3cccc4c3sc3ccccc34)cc2)cc1]

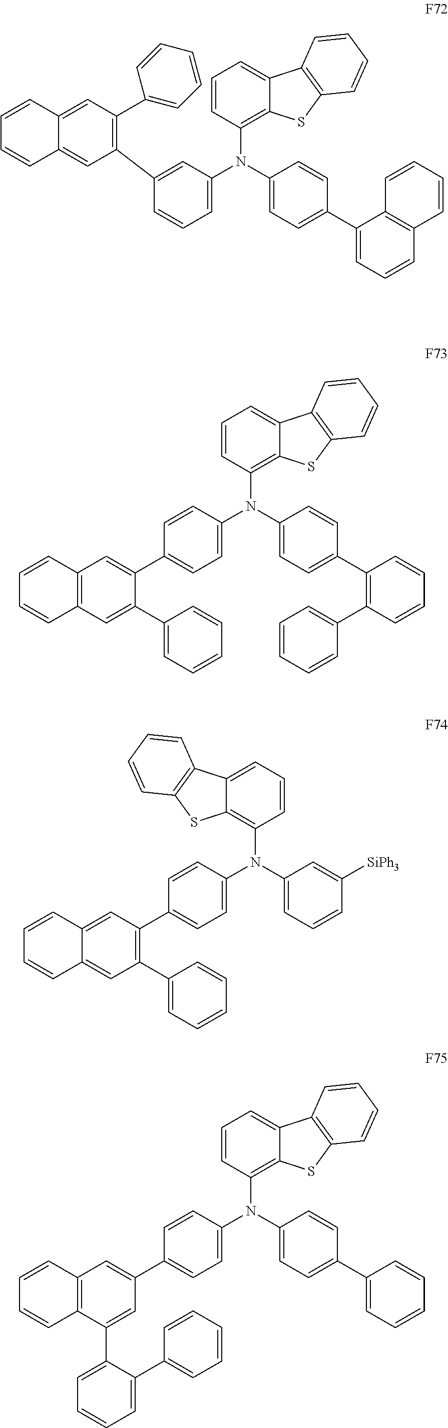

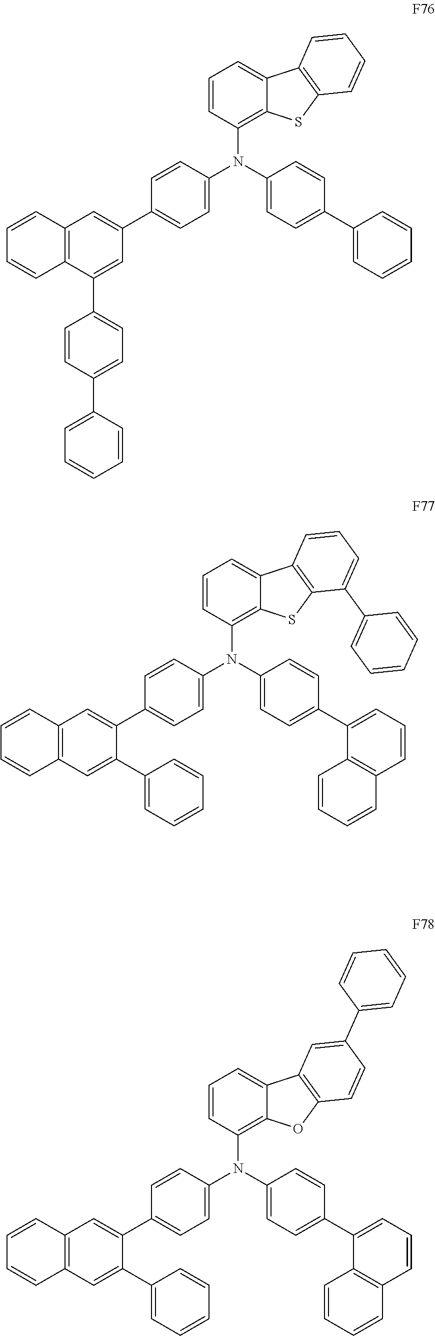 [SMILES: c1ccc(-c2ccc(-c3cc(-c4ccc(N(c5ccc(-c6ccccc6)cc5)c5cccc6c5sc5ccccc56)cc4)cc4ccccc34)cc2)cc1 ; c1ccc(-c2cc3ccccc3cc2-c2ccc(N(c3ccc(-c4cccc5ccccc45)cc3)c3cccc4c3sc3c(-c5ccccc5)cccc34)cc2)cc1 ; c1ccc(-c2ccc3oc4c(N(c5ccc(-c6cc7ccccc7cc6-c6ccccc6)cc5)c5ccc(-c6cccc7ccccc67)cc5)cccc4c3c2)cc1]

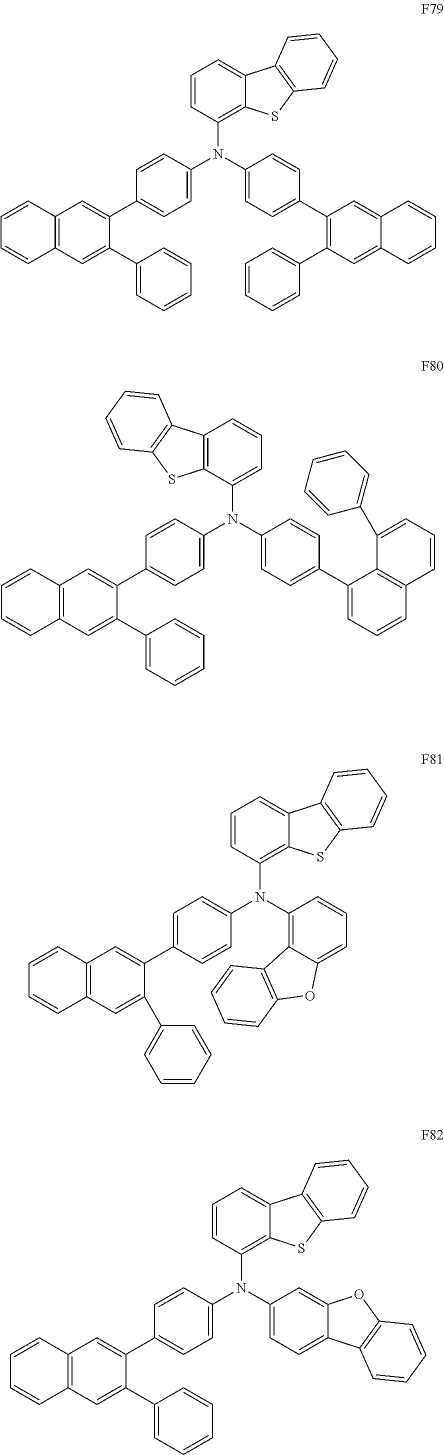 [SMILES: c1ccc(-c2cc3ccccc3cc2-c2ccc(N(c3ccc(-c4cc5ccccc5cc4-c4ccccc4)cc3)c3cccc4c3sc3ccccc34)cc2)cc1 ; c1ccc(-c2cc3ccccc3cc2-c2ccc(N(c3ccc(-c4cccc5cccc(-c6ccccc6)c45)cc3)c3cccc4c3sc3ccccc34)cc2)cc1 ; c1ccc(-c2cc3ccccc3cc2-c2ccc(N(c3cccc4c3sc3ccccc34)c3cccc4oc5ccccc5c34)cc2)cc1 ; c1ccc(-c2cc3ccccc3cc2-c2ccc(N(c3ccc4c(c3)oc3ccccc34)c3cccc4c3sc3ccccc34)cc2)cc1]

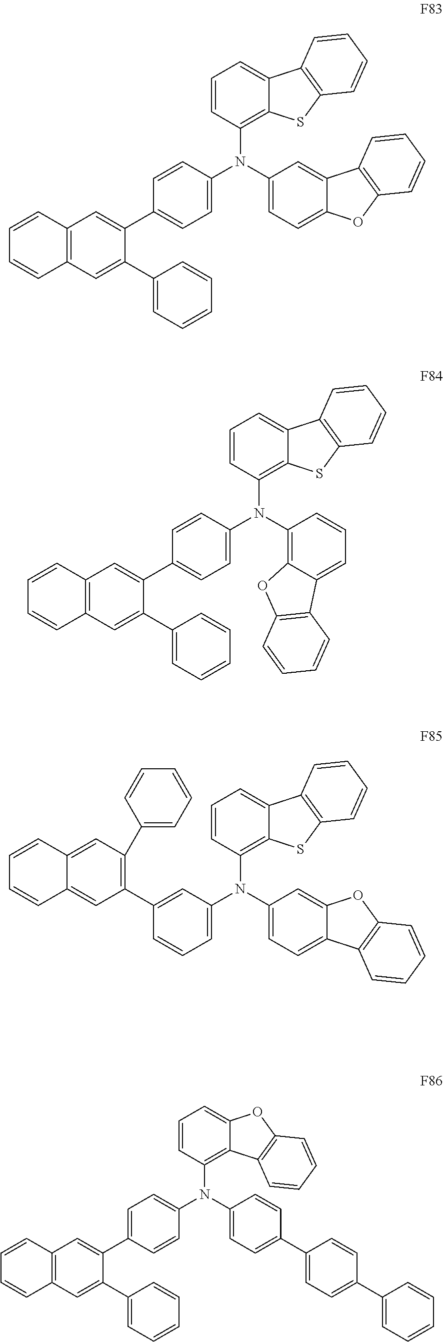 [SMILES: c1ccc(-c2cc3ccccc3cc2-c2ccc(N(c3ccc4oc5ccccc5c4c3)c3cccc4c3sc3ccccc34)cc2)cc1 ; c1ccc(-c2cc3ccccc3cc2-c2ccc(N(c3cccc4c3oc3ccccc34)c3cccc4c3sc3ccccc34)cc2)cc1 ; c1ccc(-c2cc3ccccc3cc2-c2cccc(N(c3ccc4c(c3)oc3ccccc34)c3cccc4c3sc3ccccc34)c2)cc1 ; c1ccc(-c2ccc(-c3ccc(N(c4ccc(-c5cc6ccccc6cc5-c5ccccc5)cc4)c4cccc5oc6ccccc6c45)cc3)cc2)cc1]

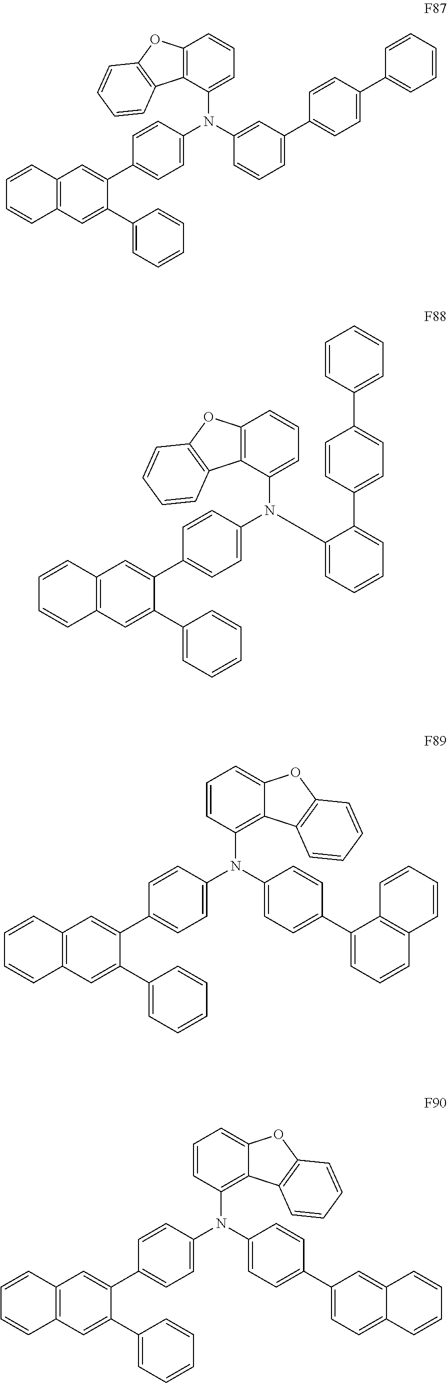 [SMILES: c1ccc(-c2ccc(-c3cccc(N(c4ccc(-c5cc6ccccc6cc5-c5ccccc5)cc4)c4cccc5oc6ccccc6c45)c3)cc2)cc1 ; c1ccc(-c2ccc(-c3ccccc3N(c3ccc(-c4cc5ccccc5cc4-c4ccccc4)cc3)c3cccc4oc5ccccc5c34)cc2)cc1 ; c1ccc(-c2cc3ccccc3cc2-c2ccc(N(c3ccc(-c4cccc5ccccc45)cc3)c3cccc4oc5ccccc5c34)cc2)cc1 ; c1ccc(-c2cc3ccccc3cc2-c2ccc(N(c3ccc(-c4ccc5ccccc5c4)cc3)c3cccc4oc5ccccc5c34)cc2)cc1]

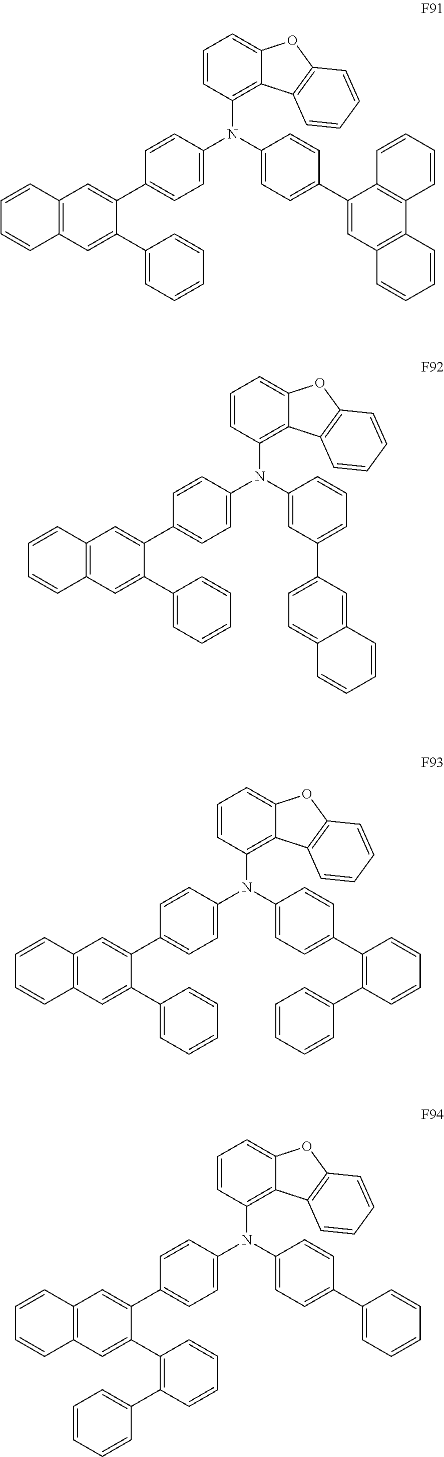 [SMILES: c1ccc(-c2cc3ccccc3cc2-c2ccc(N(c3ccc(-c4cc5ccccc5c5ccccc45)cc3)c3cccc4oc5ccccc5c34)cc2)cc1 ; c1ccc(-c2cc3ccccc3cc2-c2ccc(N(c3cccc(-c4ccc5ccccc5c4)c3)c3cccc4oc5ccccc5c34)cc2)cc1 ; c1ccc(-c2ccccc2-c2ccc(N(c3ccc(-c4cc5ccccc5cc4-c4ccccc4)cc3)c3cccc4oc5ccccc5c34)cc2)cc1 ; c1ccc(-c2ccc(N(c3ccc(-c4cc5ccccc5cc4-c4ccccc4-c4ccccc4)cc3)c3cccc4oc5ccccc5c34)cc2)cc1]

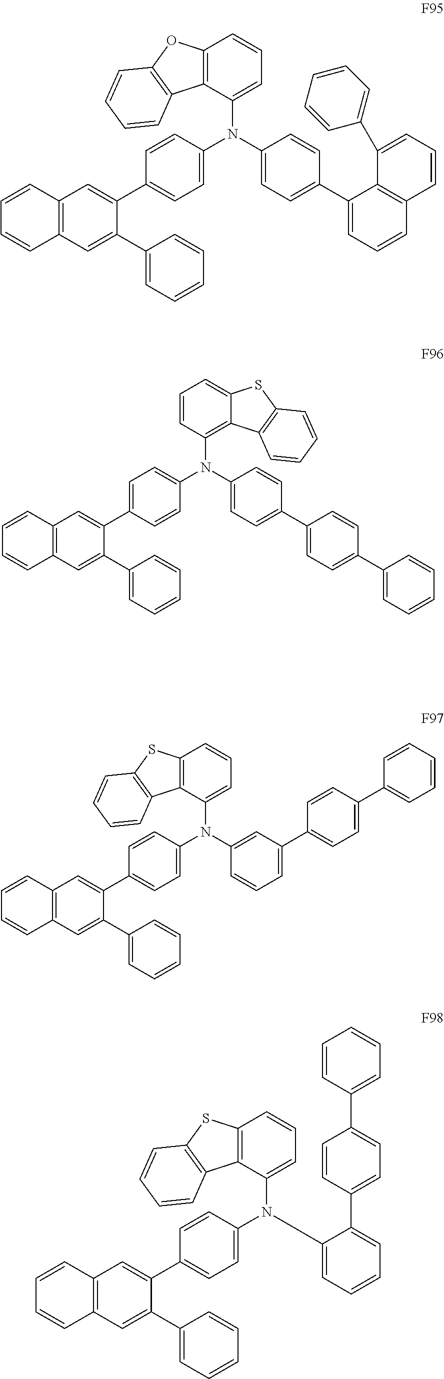 [SMILES: c1ccc(-c2cc3ccccc3cc2-c2ccc(N(c3ccc(-c4cccc5cccc(-c6ccccc6)c45)cc3)c3cccc4oc5ccccc5c34)cc2)cc1 ; c1ccc(-c2ccc(-c3ccc(N(c4ccc(-c5cc6ccccc6cc5-c5ccccc5)cc4)c4cccc5sc6ccccc6c45)cc3)cc2)cc1 ; c1ccc(-c2ccc(-c3cccc(N(c4ccc(-c5cc6ccccc6cc5-c5ccccc5)cc4)c4cccc5sc6ccccc6c45)c3)cc2)cc1 ; c1ccc(-c2ccc(-c3ccccc3N(c3ccc(-c4cc5ccccc5cc4-c4ccccc4)cc3)c3cccc4sc5ccccc5c34)cc2)cc1]

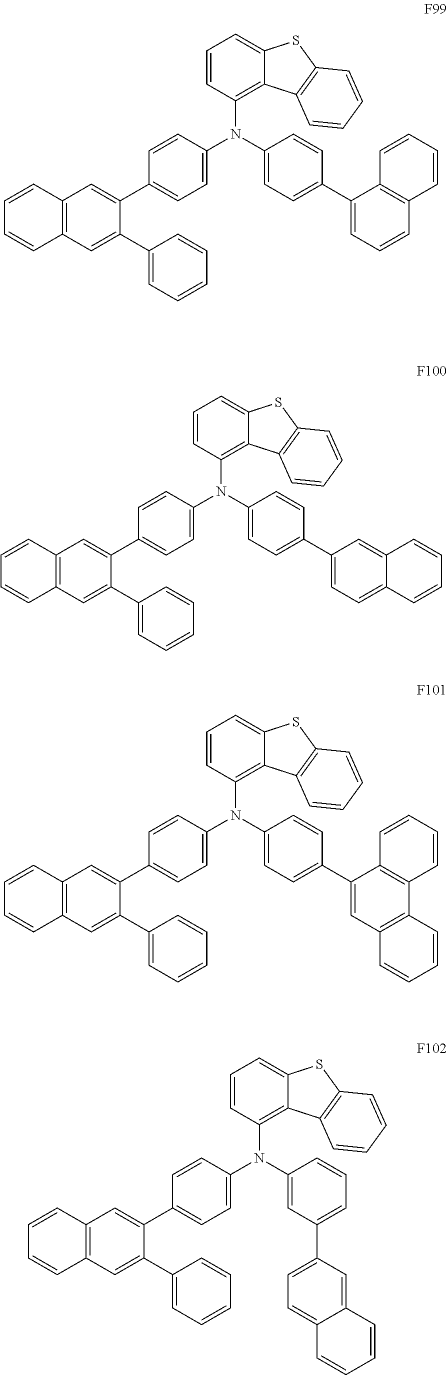 [SMILES: c1ccc(-c2cc3ccccc3cc2-c2ccc(N(c3ccc(-c4cccc5ccccc45)cc3)c3cccc4sc5ccccc5c34)cc2)cc1 ; c1ccc(-c2cc3ccccc3cc2-c2ccc(N(c3ccc(-c4ccc5ccccc5c4)cc3)c3cccc4sc5ccccc5c34)cc2)cc1 ; c1ccc(-c2cc3ccccc3cc2-c2ccc(N(c3ccc(-c4cc5ccccc5c5ccccc45)cc3)c3cccc4sc5ccccc5c34)cc2)cc1 ; c1ccc(-c2cc3ccccc3cc2-c2ccc(N(c3cccc(-c4ccc5ccccc5c4)c3)c3cccc4sc5ccccc5c34)cc2)cc1]

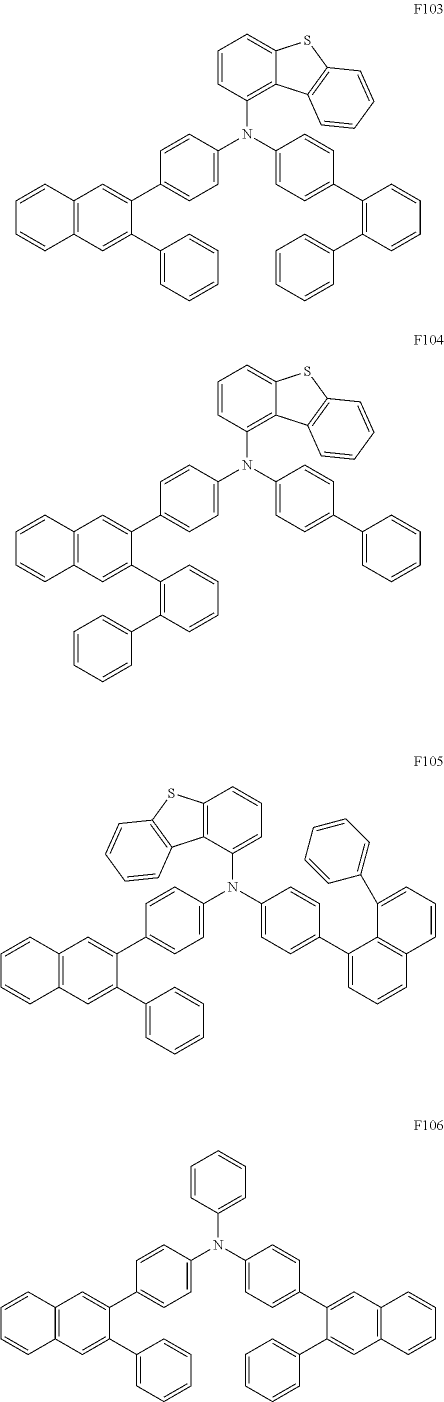 [SMILES: c1ccc(-c2ccccc2-c2ccc(N(c3ccc(-c4cc5ccccc5cc4-c4ccccc4)cc3)c3cccc4sc5ccccc5c34)cc2)cc1 ; c1ccc(-c2ccc(N(c3ccc(-c4cc5ccccc5cc4-c4ccccc4-c4ccccc4)cc3)c3cccc4sc5ccccc5c34)cc2)cc1 ; c1ccc(-c2cc3ccccc3cc2-c2ccc(N(c3ccc(-c4cccc5cccc(-c6ccccc6)c45)cc3)c3cccc4sc5ccccc5c34)cc2)cc1 ; c1ccc(-c2cc3ccccc3cc2-c2ccc(N(c3ccccc3)c3ccc(-c4cc5ccccc5cc4-c4ccccc4)cc3)cc2)cc1]

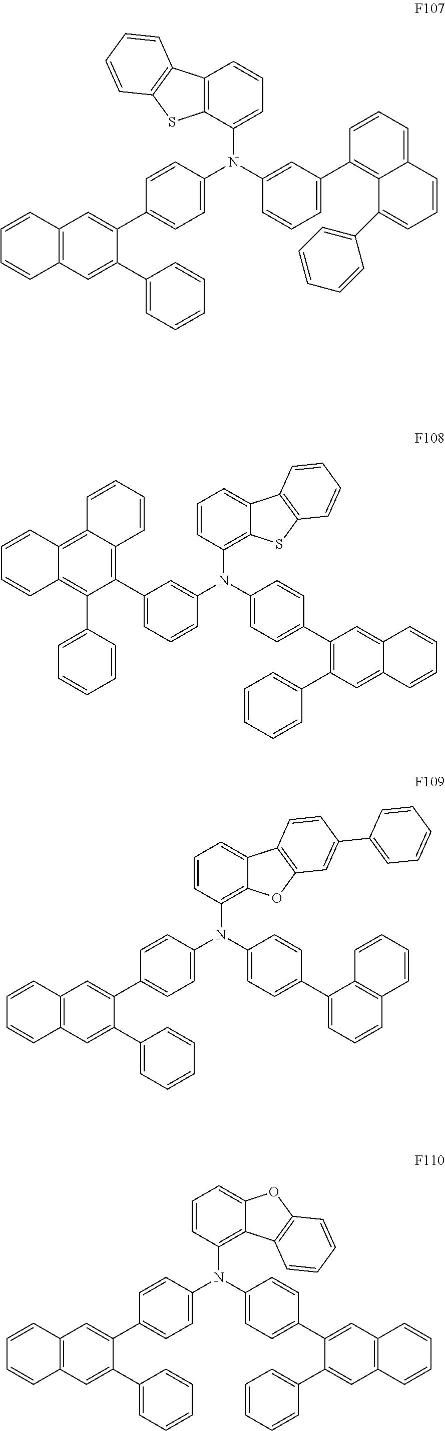 [SMILES: c1ccc(-c2cc3ccccc3cc2-c2ccc(N(c3cccc(-c4cccc5cccc(-c6ccccc6)c45)c3)c3cccc4c3sc3ccccc34)cc2)cc1 ; c1ccc(-c2cc3ccccc3cc2-c2ccc(N(c3cccc(-c4c(-c5ccccc5)c5ccccc5c5ccccc45)c3)c3cccc4c3sc3ccccc34)cc2)cc1 ; c1ccc(-c2ccc3c(c2)oc2c(N(c4ccc(-c5cc6ccccc6cc5-c5ccccc5)cc4)c4ccc(-c5cccc6ccccc56)cc4)cccc23)cc1 ; c1ccc(-c2cc3ccccc3cc2-c2ccc(N(c3ccc(-c4cc5ccccc5cc4-c4ccccc4)cc3)c3cccc4oc5ccccc5c34)cc2)cc1]

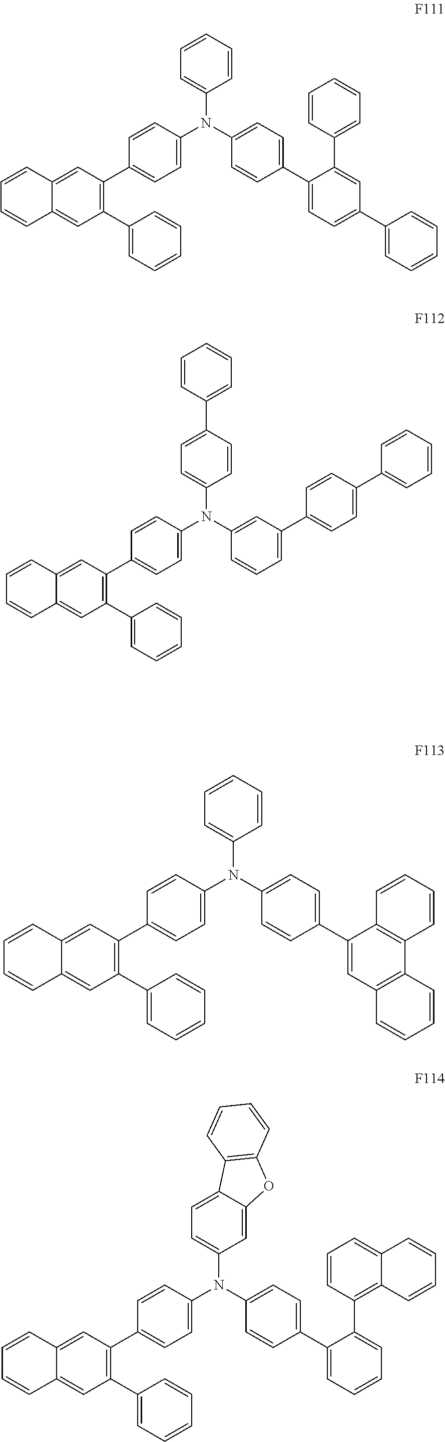 [SMILES: c1ccc(-c2ccc(-c3ccc(N(c4ccccc4)c4ccc(-c5cc6ccccc6cc5-c5ccccc5)cc4)cc3)c(-c3ccccc3)c2)cc1 ; c1ccc(-c2ccc(-c3cccc(N(c4ccc(-c5ccccc5)cc4)c4ccc(-c5cc6ccccc6cc5-c5ccccc5)cc4)c3)cc2)cc1 ; c1ccc(-c2cc3ccccc3cc2-c2ccc(N(c3ccccc3)c3ccc(-c4cc5ccccc5c5ccccc45)cc3)cc2)cc1 ; c1ccc(-c2cc3ccccc3cc2-c2ccc(N(c3ccc(-c4ccccc4-c4cccc5ccccc45)cc3)c3ccc4c(c3)oc3ccccc34)cc2)cc1]

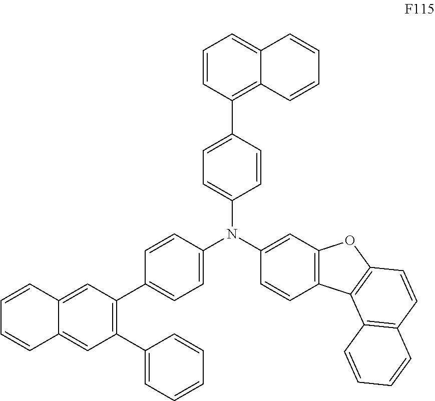 [SMILES: c1ccc(-c2cc3ccccc3cc2-c2ccc(N(c3ccc(-c4cccc5ccccc45)cc3)c3ccc4c(c3)oc3ccc5ccccc5c34)cc2)cc1]

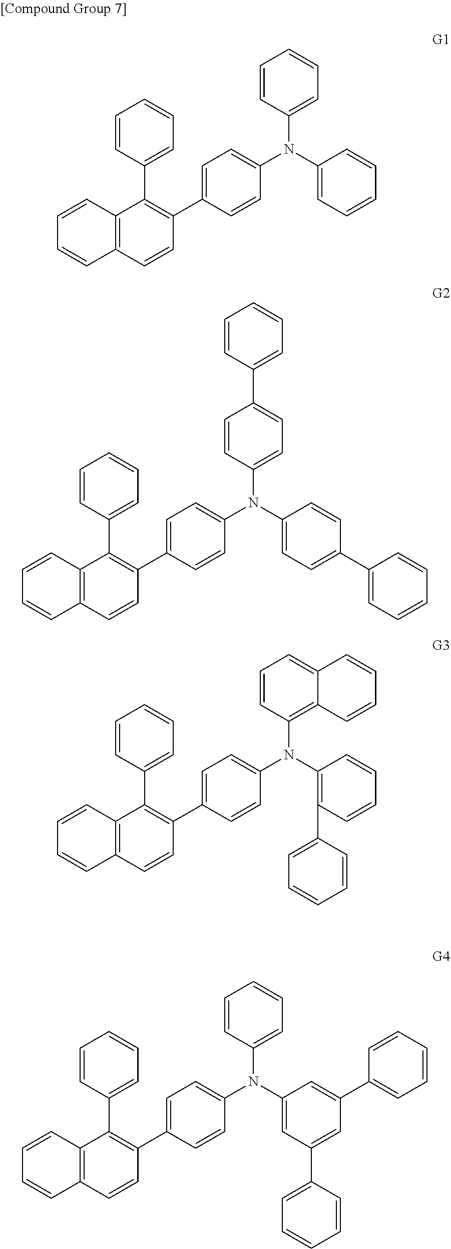 [SMILES: c1ccc(-c2c(-c3ccc(N(c4ccccc4)c4ccccc4)cc3)ccc3ccccc23)cc1 ; c1ccc(-c2ccc(N(c3ccc(-c4ccccc4)cc3)c3ccc(-c4ccc5ccccc5c4-c4ccccc4)cc3)cc2)cc1 ; c1ccc(-c2ccccc2N(c2ccc(-c3ccc4ccccc4c3-c3ccccc3)cc2)c2cccc3ccccc23)cc1 ; c1ccc(-c2cc(-c3ccccc3)cc(N(c3ccccc3)c3ccc(-c4ccc5ccccc5c4-c4ccccc4)cc3)c2)cc1]

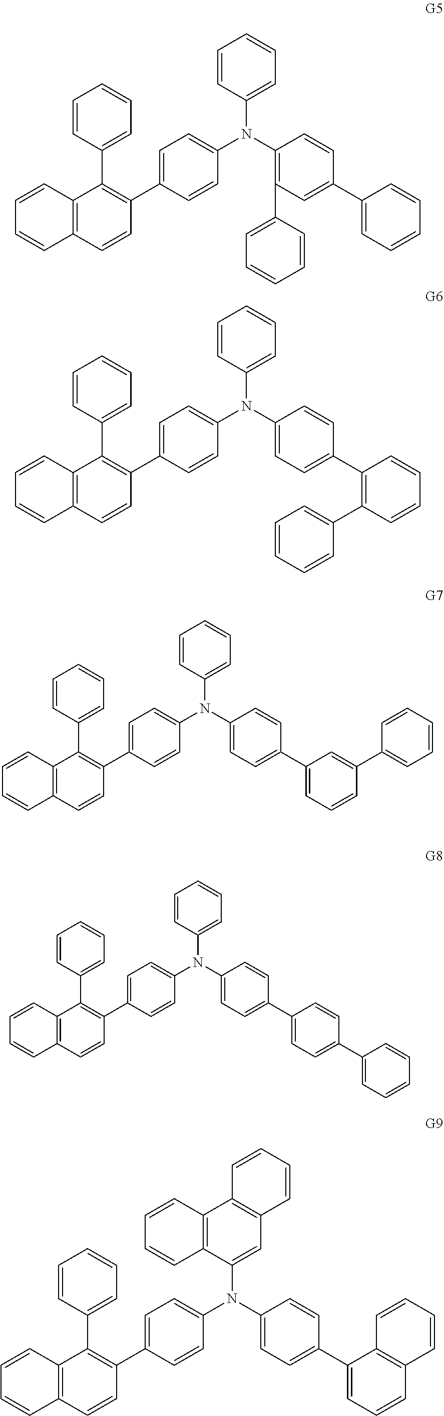

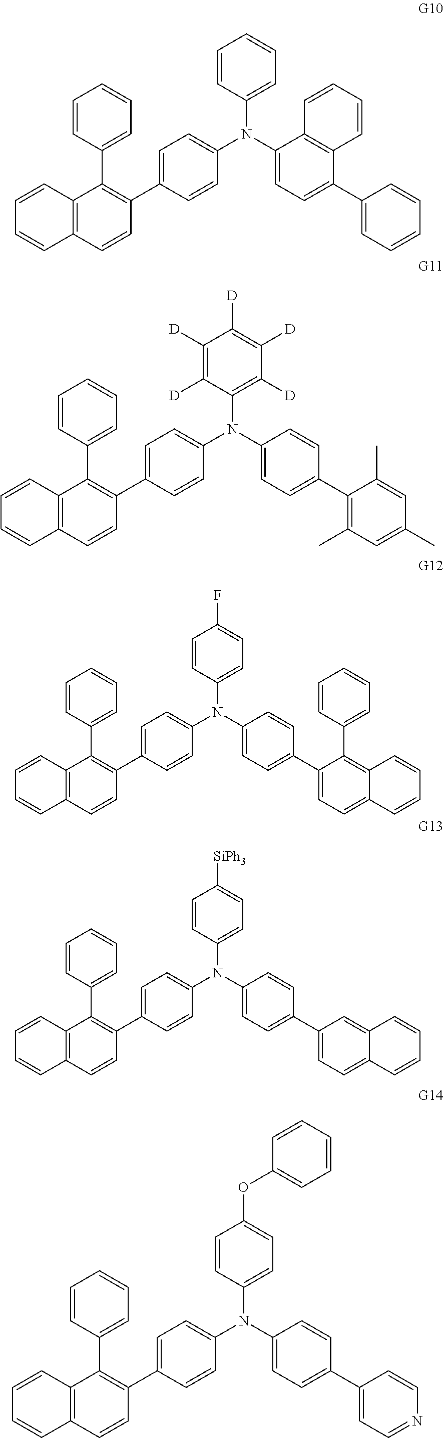

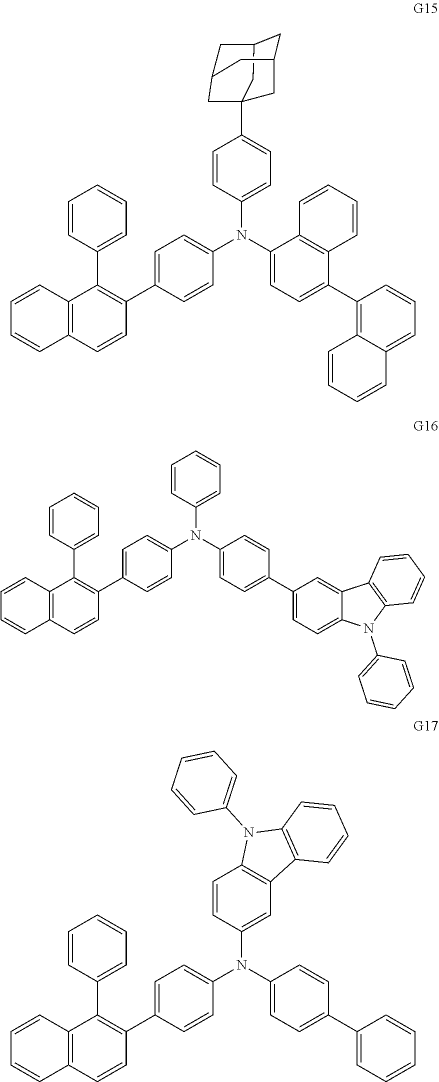 [SMILES: c1ccc(-c2c(-c3ccc(N(c4ccc(C56CC7CC(CC(C7)C5)C6)cc4)c4ccc(-c5cccc6ccccc56)c5ccccc45)cc3)ccc3ccccc23)cc1 ; c1ccc(-c2c(-c3ccc(N(c4ccccc4)c4ccc(-c5ccc6c(c5)c5ccccc5n6-c5ccccc5)cc4)cc3)ccc3ccccc23)cc1 ; c1ccc(-c2ccc(N(c3ccc(-c4ccc5ccccc5c4-c4ccccc4)cc3)c3ccc4c(c3)c3ccccc3n4-c3ccccc3)cc2)cc1]

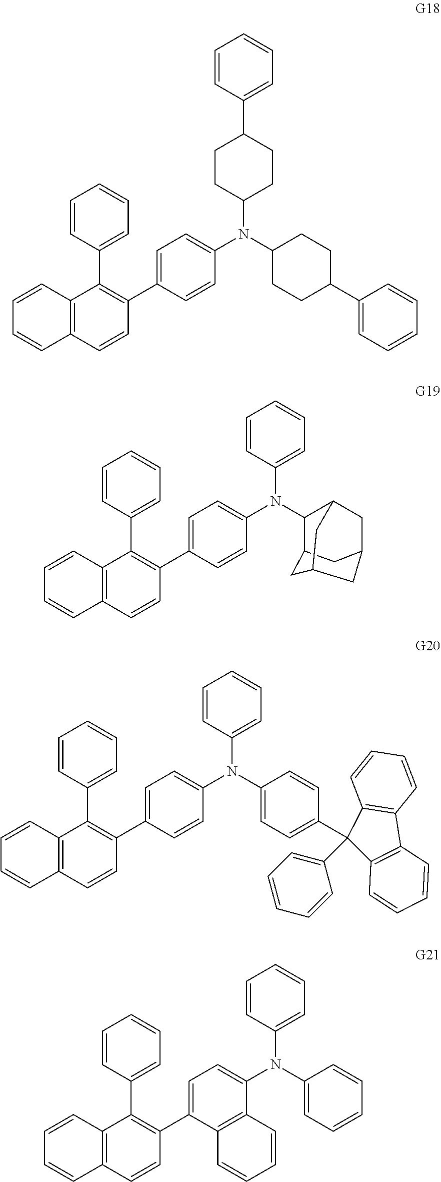 [SMILES: c1ccc(-c2c(-c3ccc(N(C4CCC(c5ccccc5)CC4)C4CCC(c5ccccc5)CC4)cc3)ccc3ccccc23)cc1 ; c1ccc(-c2c(-c3ccc(N(c4ccccc4)C4C5CC6CC(C5)CC4C6)cc3)ccc3ccccc23)cc1 ; c1ccc(-c2c(-c3ccc(N(c4ccccc4)c4ccc(C5(c6ccccc6)c6ccccc6-c6ccccc65)cc4)cc3)ccc3ccccc23)cc1 ; c1ccc(-c2c(-c3ccc(N(c4ccccc4)c4ccccc4)c4ccccc34)ccc3ccccc23)cc1]

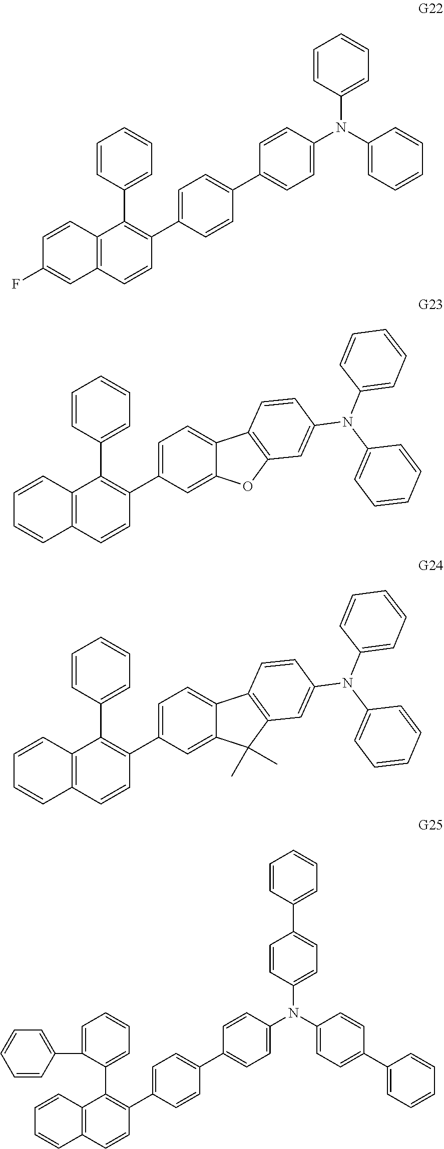 [SMILES: Fc1ccc2c(-c3ccccc3)c(-c3ccc(-c4ccc(N(c5ccccc5)c5ccccc5)cc4)cc3)ccc2c1 ; c1ccc(-c2c(-c3ccc4c(c3)oc3cc(N(c5ccccc5)c5ccccc5)ccc34)ccc3ccccc23)cc1 ; CC1(C)c2cc(-c3ccc4ccccc4c3-c3ccccc3)ccc2-c2ccc(N(c3ccccc3)c3ccccc3)cc21 ; c1ccc(-c2ccc(N(c3ccc(-c4ccccc4)cc3)c3ccc(-c4ccc(-c5ccc6ccccc6c5-c5ccccc5-c5ccccc5)cc4)cc3)cc2)cc1]

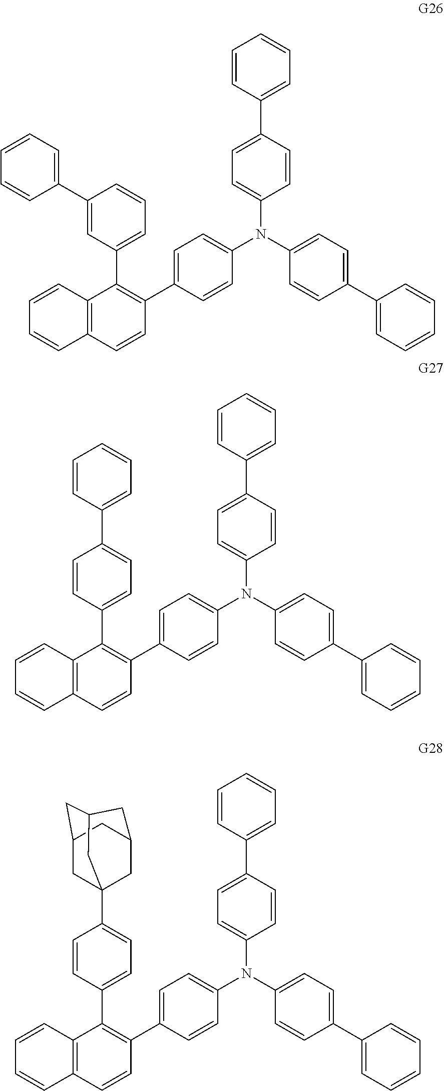 [SMILES: c1ccc(-c2ccc(N(c3ccc(-c4ccccc4)cc3)c3ccc(-c4ccc5ccccc5c4-c4cccc(-c5ccccc5)c4)cc3)cc2)cc1 ; c1ccc(-c2ccc(-c3c(-c4ccc(N(c5ccc(-c6ccccc6)cc5)c5ccc(-c6ccccc6)cc5)cc4)ccc4ccccc34)cc2)cc1 ; c1ccc(-c2ccc(N(c3ccc(-c4ccccc4)cc3)c3ccc(-c4ccc5ccccc5c4-c4ccc(C56CC7CC(CC(C7)C5)C6)cc4)cc3)cc2)cc1]

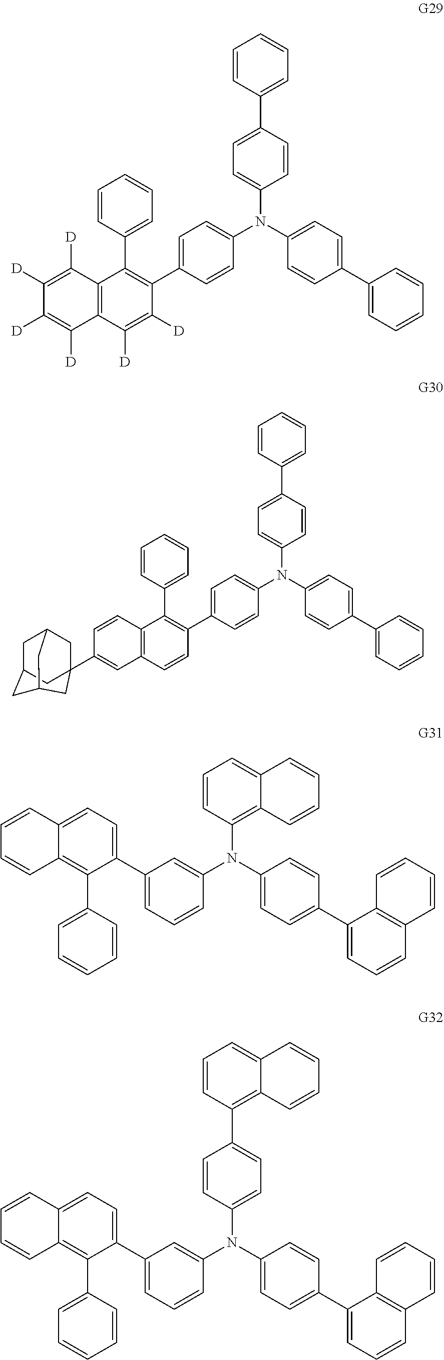 [SMILES: [2H]c1c([2H])c([2H])c2c(-c3ccccc3)c(-c3ccc(N(c4ccc(-c5ccccc5)cc4)c4ccc(-c5ccccc5)cc4)cc3)c([2H])c([2H])c2c1[2H] ; c1ccc(-c2ccc(N(c3ccc(-c4ccccc4)cc3)c3ccc(-c4ccc5cc(C67CC8CC(CC(C8)C6)C7)ccc5c4-c4ccccc4)cc3)cc2)cc1 ; c1ccc(-c2c(-c3cccc(N(c4ccc(-c5cccc6ccccc56)cc4)c4cccc5ccccc45)c3)ccc3ccccc23)cc1 ; c1ccc(-c2c(-c3cccc(N(c4ccc(-c5cccc6ccccc56)cc4)c4ccc(-c5cccc6ccccc56)cc4)c3)ccc3ccccc23)cc1]

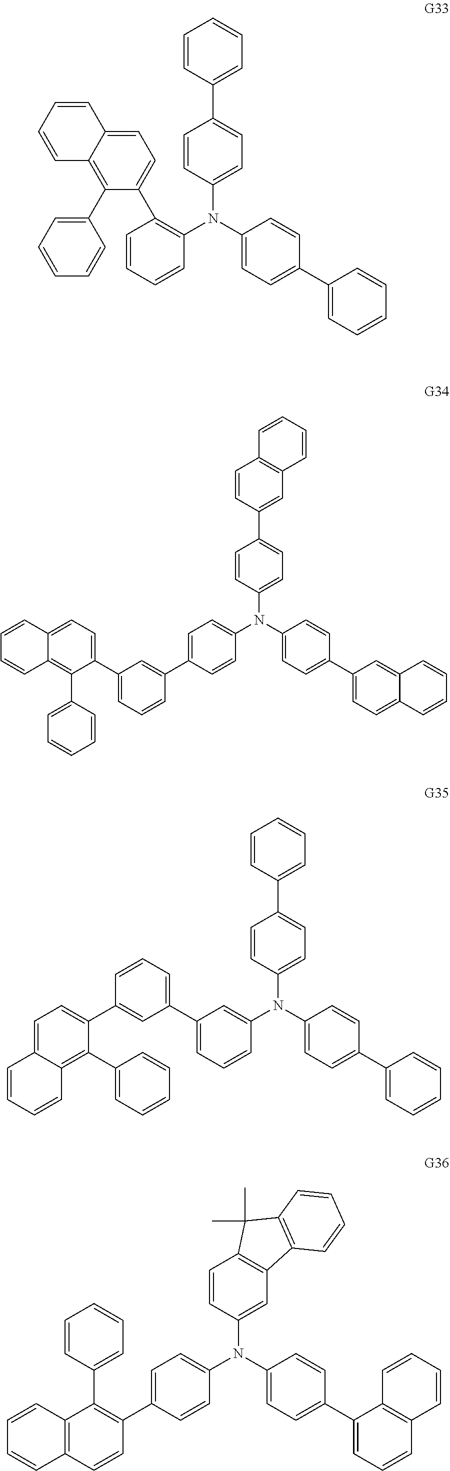 [SMILES: c1ccc(-c2ccc(N(c3ccc(-c4ccccc4)cc3)c3ccccc3-c3ccc4ccccc4c3-c3ccccc3)cc2)cc1 ; c1ccc(-c2c(-c3cccc(-c4ccc(N(c5ccc(-c6ccc7ccccc7c6)cc5)c5ccc(-c6ccc7ccccc7c6)cc5)cc4)c3)ccc3ccccc23)cc1 ; c1ccc(-c2ccc(N(c3ccc(-c4ccccc4)cc3)c3cccc(-c4cccc(-c5ccc6ccccc6c5-c5ccccc5)c4)c3)cc2)cc1 ; CC1(C)c2ccccc2-c2cc(N(c3ccc(-c4ccc5ccccc5c4-c4ccccc4)cc3)c3ccc(-c4cccc5ccccc45)cc3)ccc21]

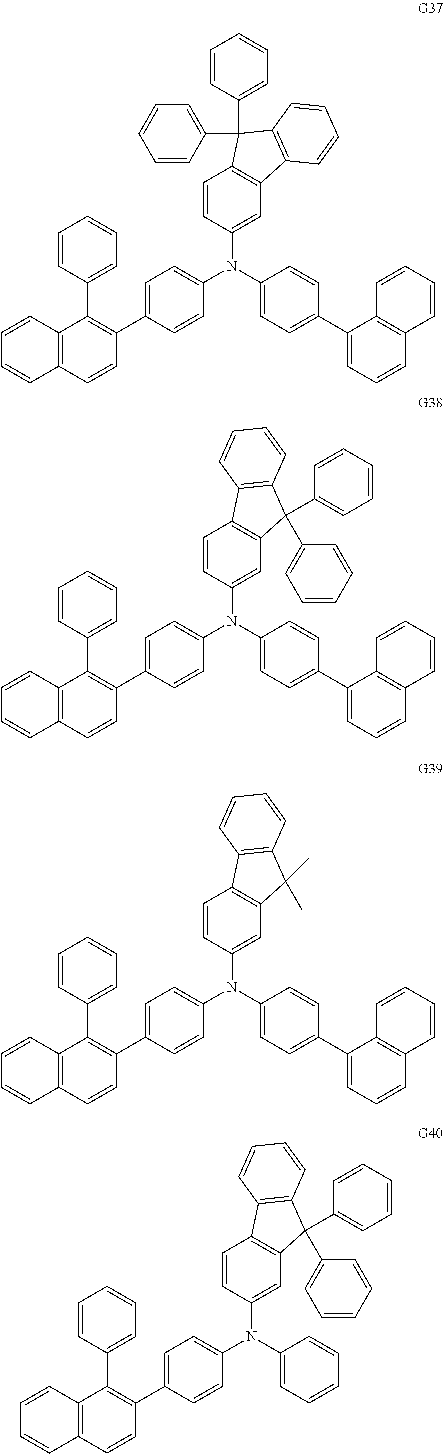 [SMILES: c1ccc(-c2c(-c3ccc(N(c4ccc(-c5cccc6ccccc56)cc4)c4ccc5c(c4)-c4ccccc4C5(c4ccccc4)c4ccccc4)cc3)ccc3ccccc23)cc1 ; c1ccc(-c2c(-c3ccc(N(c4ccc(-c5cccc6ccccc56)cc4)c4ccc5c(c4)C(c4ccccc4)(c4ccccc4)c4ccccc4-5)cc3)ccc3ccccc23)cc1 ; CC1(C)c2ccccc2-c2ccc(N(c3ccc(-c4ccc5ccccc5c4-c4ccccc4)cc3)c3ccc(-c4cccc5ccccc45)cc3)cc21 ; c1ccc(-c2c(-c3ccc(N(c4ccccc4)c4ccc5c(c4)C(c4ccccc4)(c4ccccc4)c4ccccc4-5)cc3)ccc3ccccc23)cc1]

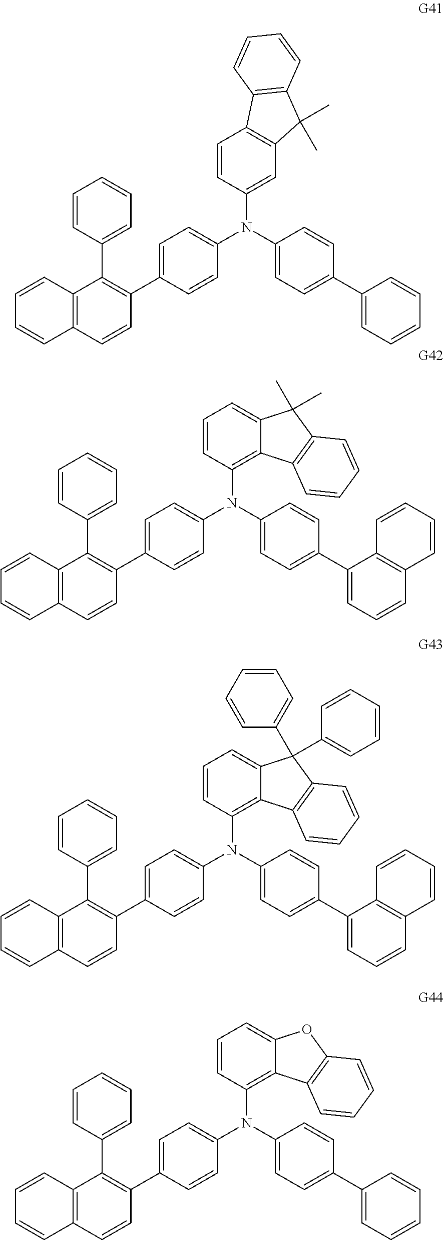 [SMILES: CC1(C)c2ccccc2-c2ccc(N(c3ccc(-c4ccccc4)cc3)c3ccc(-c4ccc5ccccc5c4-c4ccccc4)cc3)cc21 ; CC1(C)c2ccccc2-c2c(N(c3ccc(-c4ccc5ccccc5c4-c4ccccc4)cc3)c3ccc(-c4cccc5ccccc45)cc3)cccc21 ; c1ccc(-c2c(-c3ccc(N(c4ccc(-c5cccc6ccccc56)cc4)c4cccc5c4-c4ccccc4C5(c4ccccc4)c4ccccc4)cc3)ccc3ccccc23)cc1 ; c1ccc(-c2ccc(N(c3ccc(-c4ccc5ccccc5c4-c4ccccc4)cc3)c3cccc4oc5ccccc5c34)cc2)cc1]

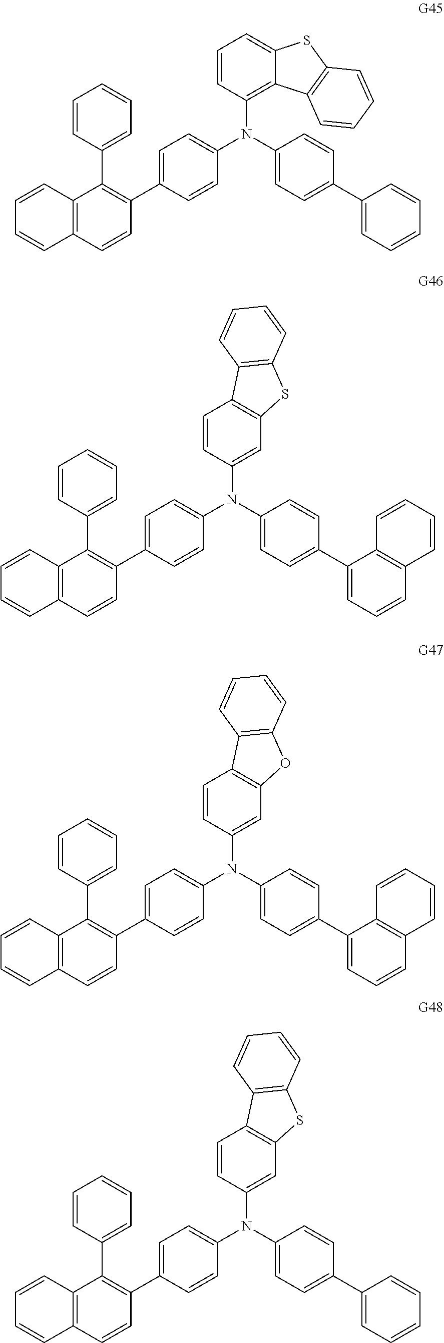 [SMILES: c1ccc(-c2ccc(N(c3ccc(-c4ccc5ccccc5c4-c4ccccc4)cc3)c3cccc4sc5ccccc5c34)cc2)cc1 ; c1ccc(-c2c(-c3ccc(N(c4ccc(-c5cccc6ccccc56)cc4)c4ccc5c(c4)sc4ccccc45)cc3)ccc3ccccc23)cc1 ; c1ccc(-c2c(-c3ccc(N(c4ccc(-c5cccc6ccccc56)cc4)c4ccc5c(c4)oc4ccccc45)cc3)ccc3ccccc23)cc1 ; c1ccc(-c2ccc(N(c3ccc(-c4ccc5ccccc5c4-c4ccccc4)cc3)c3ccc4c(c3)sc3ccccc34)cc2)cc1]

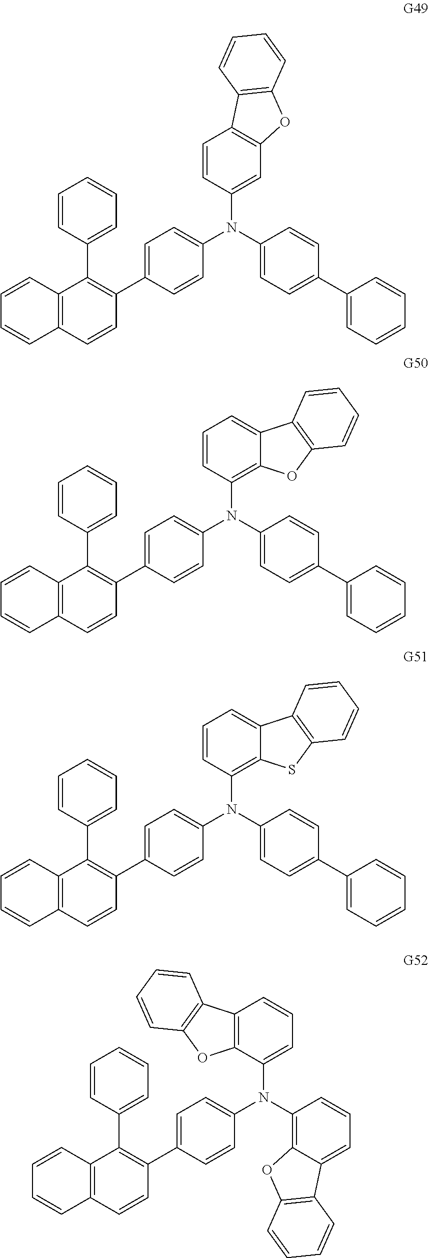 [SMILES: c1ccc(-c2ccc(N(c3ccc(-c4ccc5ccccc5c4-c4ccccc4)cc3)c3ccc4c(c3)oc3ccccc34)cc2)cc1 ; c1ccc(-c2ccc(N(c3ccc(-c4ccc5ccccc5c4-c4ccccc4)cc3)c3cccc4c3oc3ccccc34)cc2)cc1 ; c1ccc(-c2ccc(N(c3ccc(-c4ccc5ccccc5c4-c4ccccc4)cc3)c3cccc4c3sc3ccccc34)cc2)cc1 ; c1ccc(-c2c(-c3ccc(N(c4cccc5c4oc4ccccc45)c4cccc5c4oc4ccccc45)cc3)ccc3ccccc23)cc1]

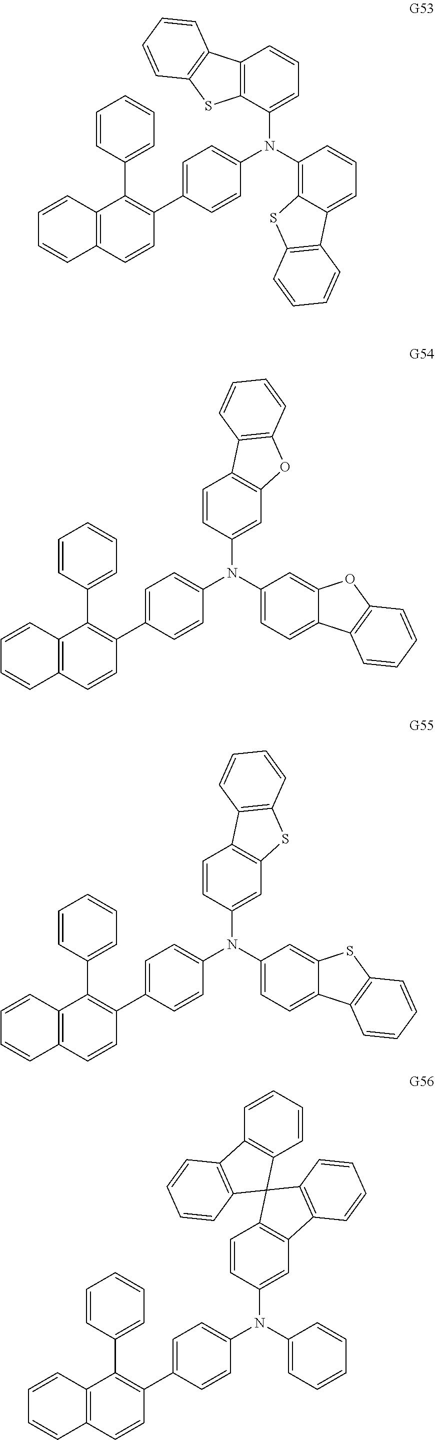 [SMILES: c1ccc(-c2c(-c3ccc(N(c4cccc5c4sc4ccccc45)c4cccc5c4sc4ccccc45)cc3)ccc3ccccc23)cc1 ; c1ccc(-c2c(-c3ccc(N(c4ccc5c(c4)oc4ccccc45)c4ccc5c(c4)oc4ccccc45)cc3)ccc3ccccc23)cc1 ; c1ccc(-c2c(-c3ccc(N(c4ccc5c(c4)sc4ccccc45)c4ccc5c(c4)sc4ccccc45)cc3)ccc3ccccc23)cc1 ; c1ccc(-c2c(-c3ccc(N(c4ccccc4)c4ccc5c(c4)-c4ccccc4C54c5ccccc5-c5ccccc54)cc3)ccc3ccccc23)cc1]

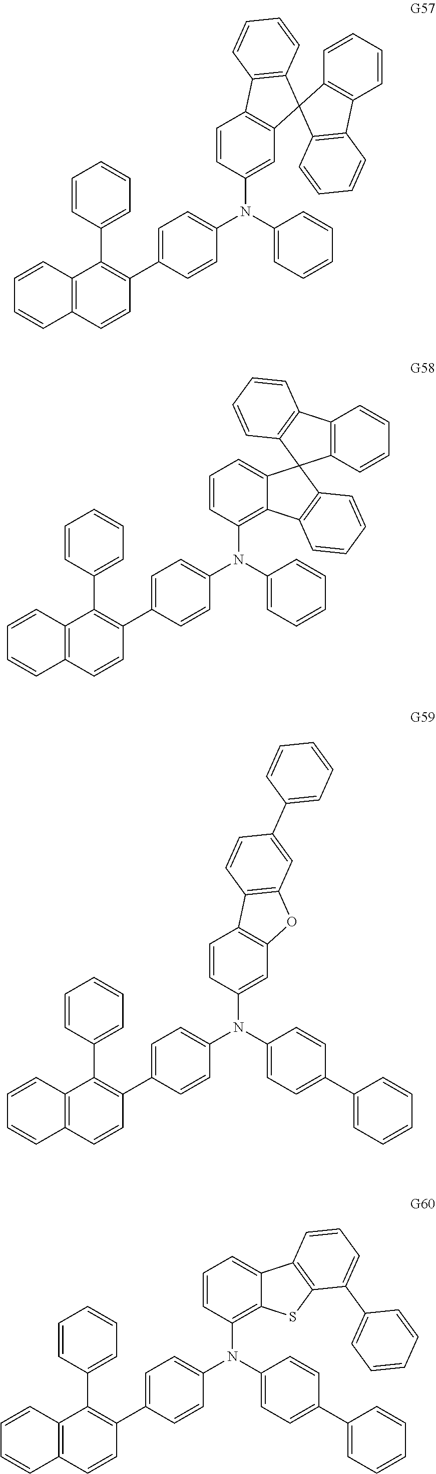 [SMILES: c1ccc(-c2c(-c3ccc(N(c4ccccc4)c4ccc5c(c4)C4(c6ccccc6-c6ccccc64)c4ccccc4-5)cc3)ccc3ccccc23)cc1 ; c1ccc(-c2c(-c3ccc(N(c4ccccc4)c4cccc5c4-c4ccccc4C54c5ccccc5-c5ccccc54)cc3)ccc3ccccc23)cc1 ; c1ccc(-c2ccc(N(c3ccc(-c4ccc5ccccc5c4-c4ccccc4)cc3)c3ccc4c(c3)oc3cc(-c5ccccc5)ccc34)cc2)cc1 ; c1ccc(-c2ccc(N(c3ccc(-c4ccc5ccccc5c4-c4ccccc4)cc3)c3cccc4c3sc3c(-c5ccccc5)cccc34)cc2)cc1]

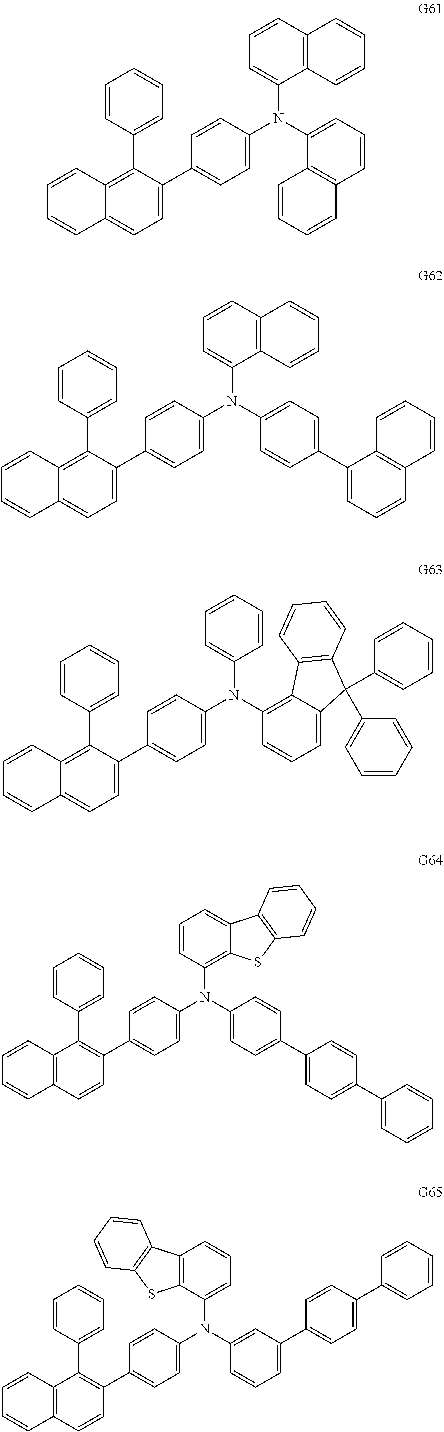 [SMILES: c1ccc(-c2c(-c3ccc(N(c4cccc5ccccc45)c4cccc5ccccc45)cc3)ccc3ccccc23)cc1 ; c1ccc(-c2c(-c3ccc(N(c4ccc(-c5cccc6ccccc56)cc4)c4cccc5ccccc45)cc3)ccc3ccccc23)cc1 ; c1ccc(-c2c(-c3ccc(N(c4ccccc4)c4cccc5c4-c4ccccc4C5(c4ccccc4)c4ccccc4)cc3)ccc3ccccc23)cc1 ; c1ccc(-c2ccc(-c3ccc(N(c4ccc(-c5ccc6ccccc6c5-c5ccccc5)cc4)c4cccc5c4sc4ccccc45)cc3)cc2)cc1 ; c1ccc(-c2ccc(-c3cccc(N(c4ccc(-c5ccc6ccccc6c5-c5ccccc5)cc4)c4cccc5c4sc4ccccc45)c3)cc2)cc1]

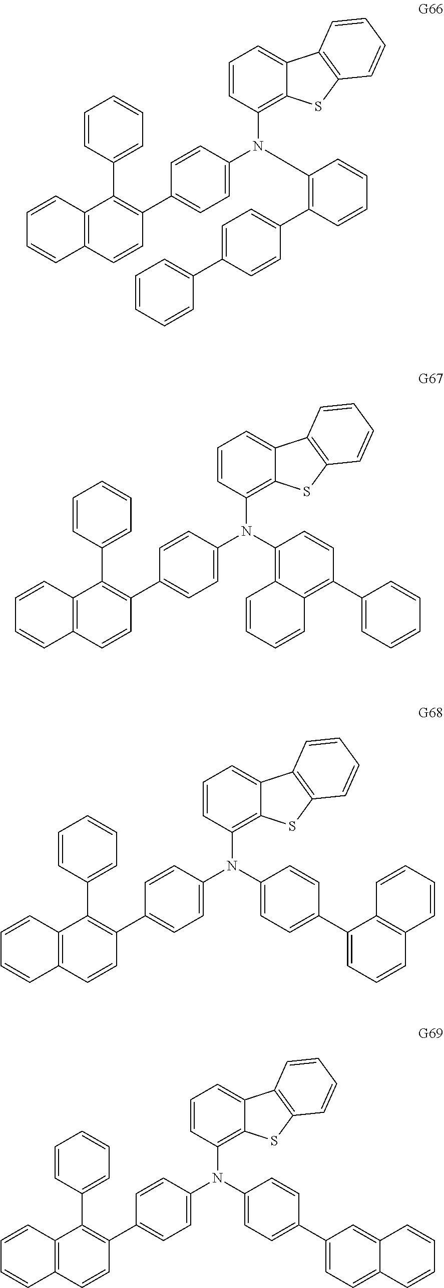 [SMILES: c1ccc(-c2ccc(-c3ccccc3N(c3ccc(-c4ccc5ccccc5c4-c4ccccc4)cc3)c3cccc4c3sc3ccccc34)cc2)cc1 ; c1ccc(-c2c(-c3ccc(N(c4ccc(-c5ccccc5)c5ccccc45)c4cccc5c4sc4ccccc45)cc3)ccc3ccccc23)cc1 ; c1ccc(-c2c(-c3ccc(N(c4ccc(-c5cccc6ccccc56)cc4)c4cccc5c4sc4ccccc45)cc3)ccc3ccccc23)cc1 ; c1ccc(-c2c(-c3ccc(N(c4ccc(-c5ccc6ccccc6c5)cc4)c4cccc5c4sc4ccccc45)cc3)ccc3ccccc23)cc1]

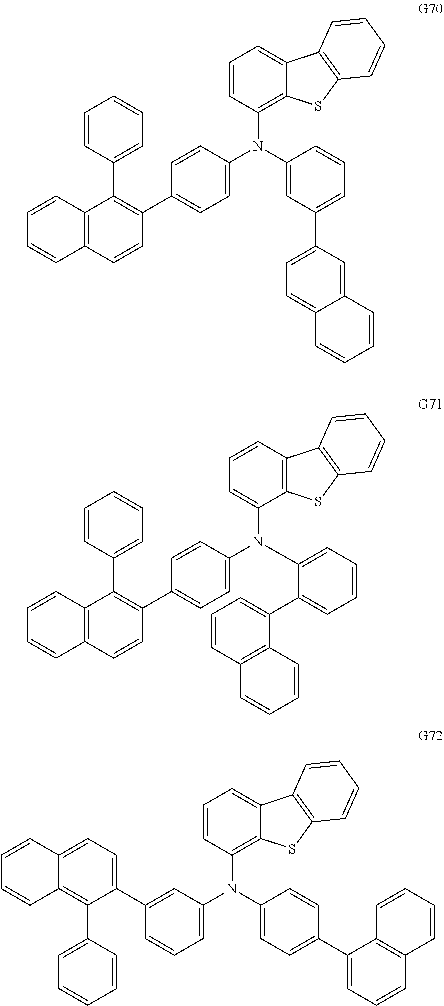 [SMILES: c1ccc(-c2c(-c3ccc(N(c4cccc(-c5ccc6ccccc6c5)c4)c4cccc5c4sc4ccccc45)cc3)ccc3ccccc23)cc1 ; c1ccc(-c2c(-c3ccc(N(c4ccccc4-c4cccc5ccccc45)c4cccc5c4sc4ccccc45)cc3)ccc3ccccc23)cc1 ; c1ccc(-c2c(-c3cccc(N(c4ccc(-c5cccc6ccccc56)cc4)c4cccc5c4sc4ccccc45)c3)ccc3ccccc23)cc1]

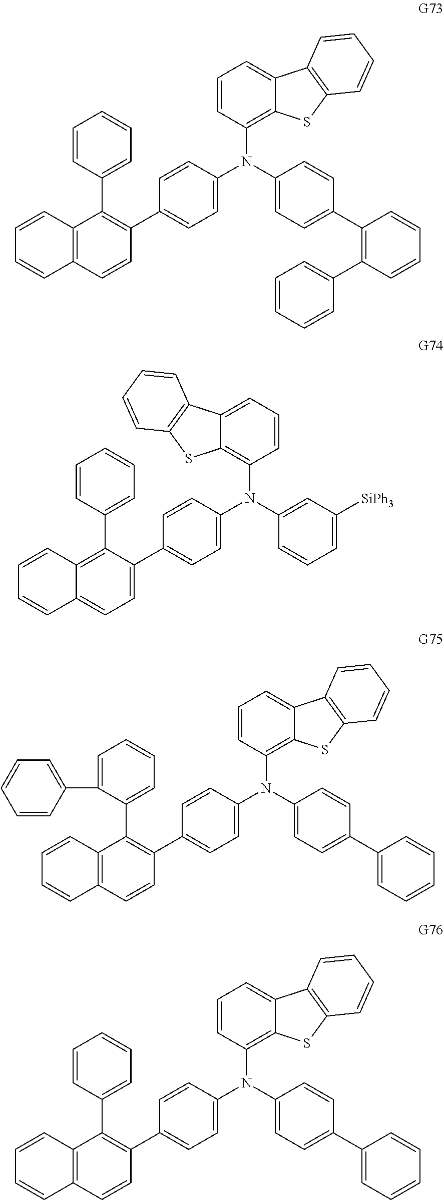

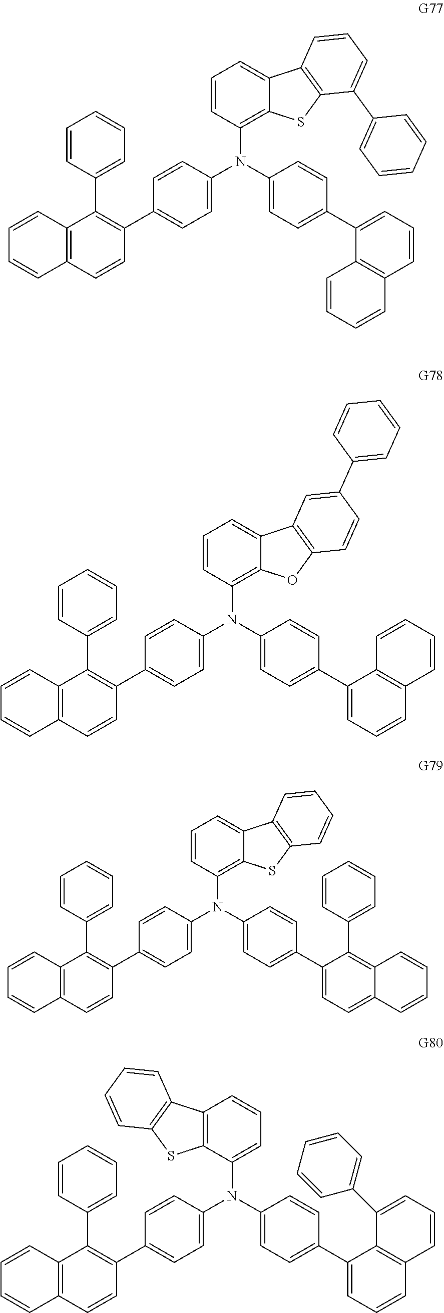 [SMILES: c1ccc(-c2c(-c3ccc(N(c4ccc(-c5cccc6ccccc56)cc4)c4cccc5c4sc4c(-c6ccccc6)cccc45)cc3)ccc3ccccc23)cc1 ; c1ccc(-c2ccc3oc4c(N(c5ccc(-c6ccc7ccccc7c6-c6ccccc6)cc5)c5ccc(-c6cccc7ccccc67)cc5)cccc4c3c2)cc1 ; c1ccc(-c2c(-c3ccc(N(c4ccc(-c5ccc6ccccc6c5-c5ccccc5)cc4)c4cccc5c4sc4ccccc45)cc3)ccc3ccccc23)cc1 ; c1ccc(-c2c(-c3ccc(N(c4ccc(-c5cccc6cccc(-c7ccccc7)c56)cc4)c4cccc5c4sc4ccccc45)cc3)ccc3ccccc23)cc1]

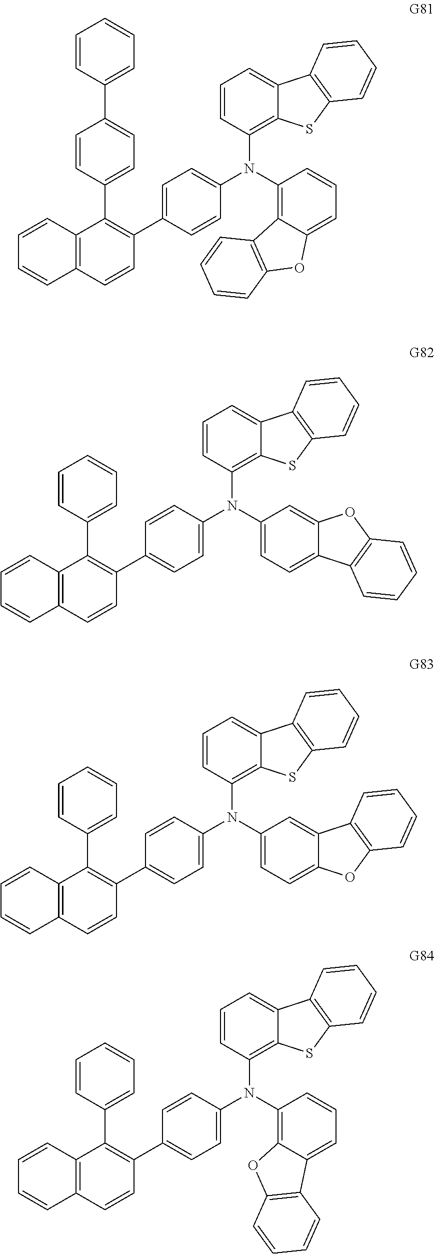 [SMILES: c1ccc(-c2ccc(-c3c(-c4ccc(N(c5cccc6c5sc5ccccc56)c5cccc6oc7ccccc7c56)cc4)ccc4ccccc34)cc2)cc1 ; c1ccc(-c2c(-c3ccc(N(c4ccc5c(c4)oc4ccccc45)c4cccc5c4sc4ccccc45)cc3)ccc3ccccc23)cc1 ; c1ccc(-c2c(-c3ccc(N(c4ccc5oc6ccccc6c5c4)c4cccc5c4sc4ccccc45)cc3)ccc3ccccc23)cc1 ; c1ccc(-c2c(-c3ccc(N(c4cccc5c4oc4ccccc45)c4cccc5c4sc4ccccc45)cc3)ccc3ccccc23)cc1]

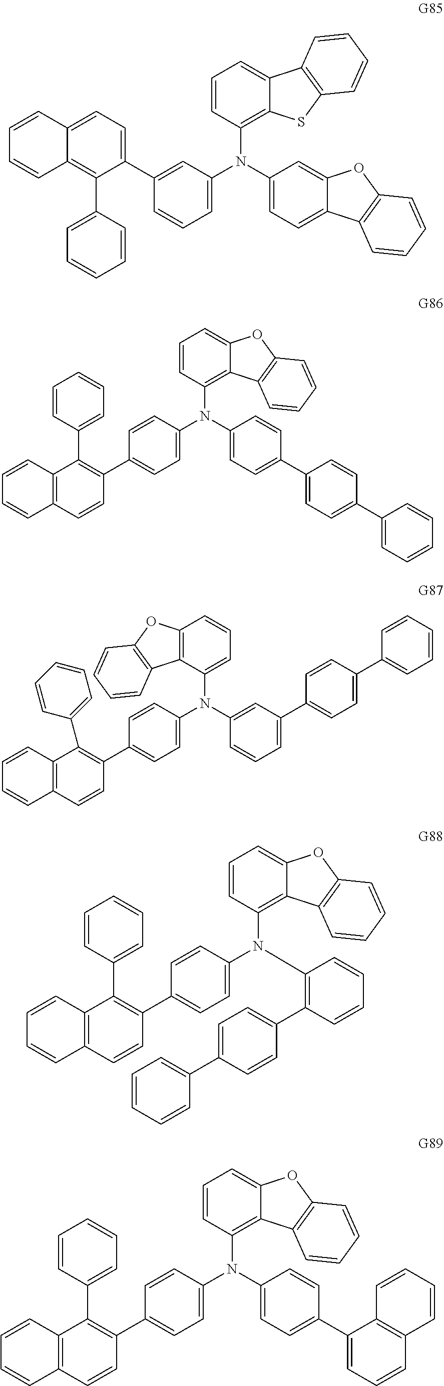 [SMILES: c1ccc(-c2c(-c3cccc(N(c4ccc5c(c4)oc4ccccc45)c4cccc5c4sc4ccccc45)c3)ccc3ccccc23)cc1 ; c1ccc(-c2ccc(-c3ccc(N(c4ccc(-c5ccc6ccccc6c5-c5ccccc5)cc4)c4cccc5oc6ccccc6c45)cc3)cc2)cc1 ; c1ccc(-c2ccc(-c3cccc(N(c4ccc(-c5ccc6ccccc6c5-c5ccccc5)cc4)c4cccc5oc6ccccc6c45)c3)cc2)cc1 ; c1ccc(-c2ccc(-c3ccccc3N(c3ccc(-c4ccc5ccccc5c4-c4ccccc4)cc3)c3cccc4oc5ccccc5c34)cc2)cc1 ; c1ccc(-c2c(-c3ccc(N(c4ccc(-c5cccc6ccccc56)cc4)c4cccc5oc6ccccc6c45)cc3)ccc3ccccc23)cc1]

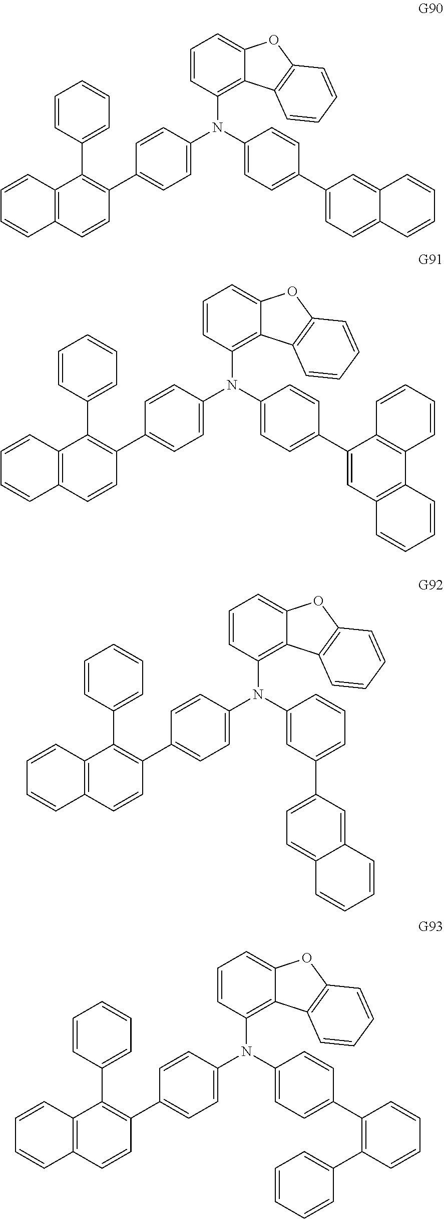 [SMILES: c1ccc(-c2c(-c3ccc(N(c4ccc(-c5ccc6ccccc6c5)cc4)c4cccc5oc6ccccc6c45)cc3)ccc3ccccc23)cc1 ; c1ccc(-c2c(-c3ccc(N(c4ccc(-c5cc6ccccc6c6ccccc56)cc4)c4cccc5oc6ccccc6c45)cc3)ccc3ccccc23)cc1 ; c1ccc(-c2c(-c3ccc(N(c4cccc(-c5ccc6ccccc6c5)c4)c4cccc5oc6ccccc6c45)cc3)ccc3ccccc23)cc1 ; c1ccc(-c2ccccc2-c2ccc(N(c3ccc(-c4ccc5ccccc5c4-c4ccccc4)cc3)c3cccc4oc5ccccc5c34)cc2)cc1]

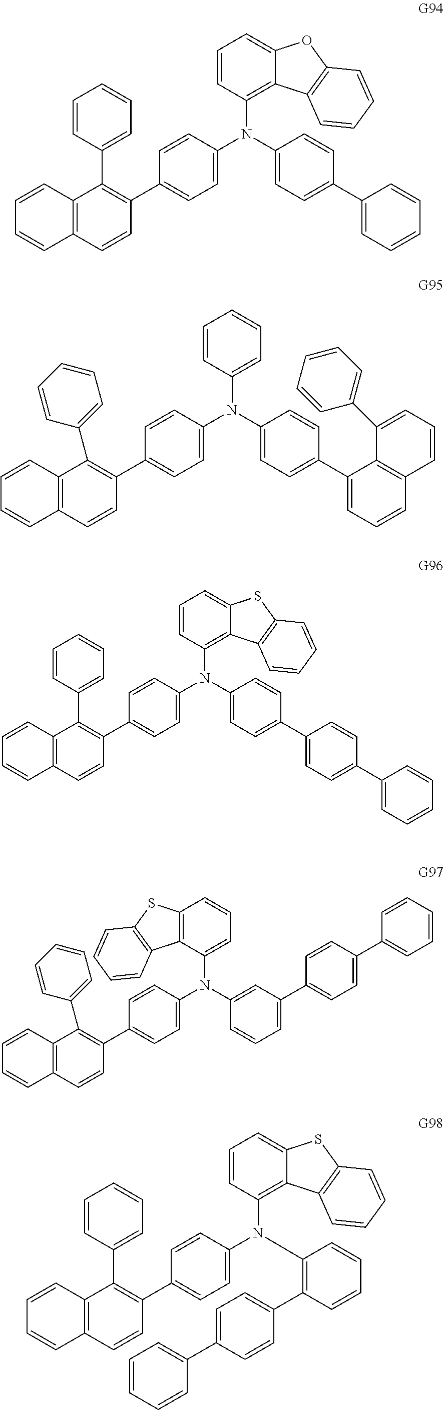 [SMILES: c1ccc(-c2ccc(N(c3ccc(-c4ccc5ccccc5c4-c4ccccc4)cc3)c3cccc4oc5ccccc5c34)cc2)cc1 ; c1ccc(-c2c(-c3ccc(N(c4ccccc4)c4ccc(-c5cccc6cccc(-c7ccccc7)c56)cc4)cc3)ccc3ccccc23)cc1 ; c1ccc(-c2ccc(-c3ccc(N(c4ccc(-c5ccc6ccccc6c5-c5ccccc5)cc4)c4cccc5sc6ccccc6c45)cc3)cc2)cc1 ; c1ccc(-c2ccc(-c3cccc(N(c4ccc(-c5ccc6ccccc6c5-c5ccccc5)cc4)c4cccc5sc6ccccc6c45)c3)cc2)cc1 ; c1ccc(-c2ccc(-c3ccccc3N(c3ccc(-c4ccc5ccccc5c4-c4ccccc4)cc3)c3cccc4sc5ccccc5c34)cc2)cc1]

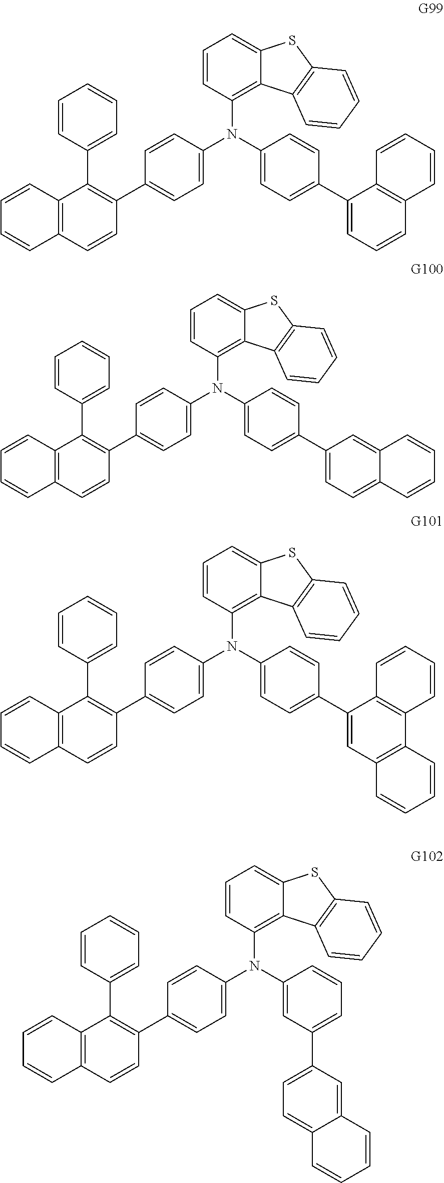 [SMILES: c1ccc(-c2c(-c3ccc(N(c4ccc(-c5cccc6ccccc56)cc4)c4cccc5sc6ccccc6c45)cc3)ccc3ccccc23)cc1 ; c1ccc(-c2c(-c3ccc(N(c4ccc(-c5ccc6ccccc6c5)cc4)c4cccc5sc6ccccc6c45)cc3)ccc3ccccc23)cc1 ; c1ccc(-c2c(-c3ccc(N(c4ccc(-c5cc6ccccc6c6ccccc56)cc4)c4cccc5sc6ccccc6c45)cc3)ccc3ccccc23)cc1 ; c1ccc(-c2c(-c3ccc(N(c4cccc(-c5ccc6ccccc6c5)c4)c4cccc5sc6ccccc6c45)cc3)ccc3ccccc23)cc1]

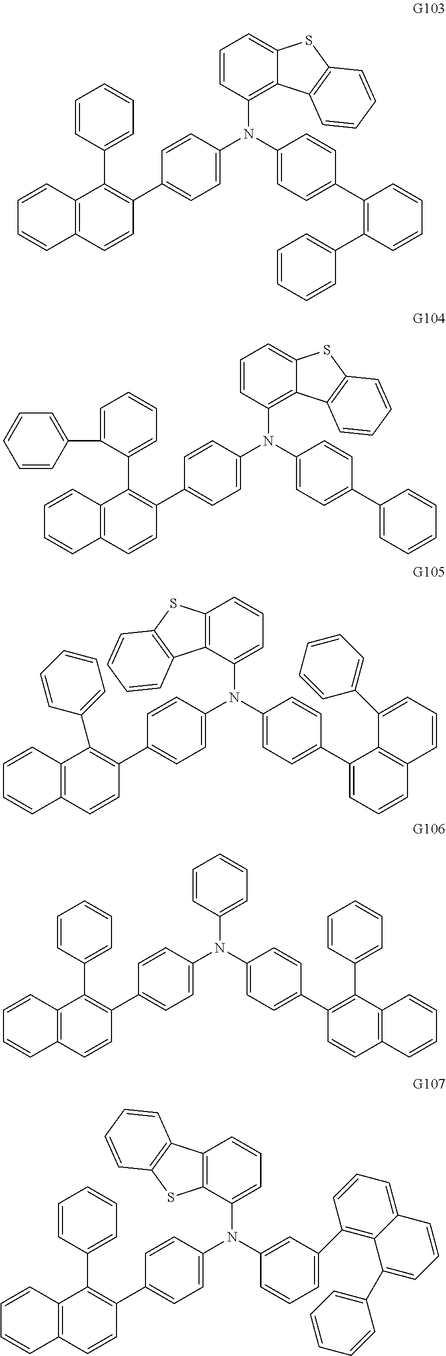 [SMILES: c1ccc(-c2ccccc2-c2ccc(N(c3ccc(-c4ccc5ccccc5c4-c4ccccc4)cc3)c3cccc4sc5ccccc5c34)cc2)cc1 ; c1ccc(-c2ccc(N(c3ccc(-c4ccc5ccccc5c4-c4ccccc4-c4ccccc4)cc3)c3cccc4sc5ccccc5c34)cc2)cc1 ; c1ccc(-c2c(-c3ccc(N(c4ccc(-c5cccc6cccc(-c7ccccc7)c56)cc4)c4cccc5sc6ccccc6c45)cc3)ccc3ccccc23)cc1 ; c1ccc(-c2c(-c3ccc(N(c4ccccc4)c4ccc(-c5ccc6ccccc6c5-c5ccccc5)cc4)cc3)ccc3ccccc23)cc1 ; c1ccc(-c2c(-c3ccc(N(c4cccc(-c5cccc6cccc(-c7ccccc7)c56)c4)c4cccc5c4sc4ccccc45)cc3)ccc3ccccc23)cc1]

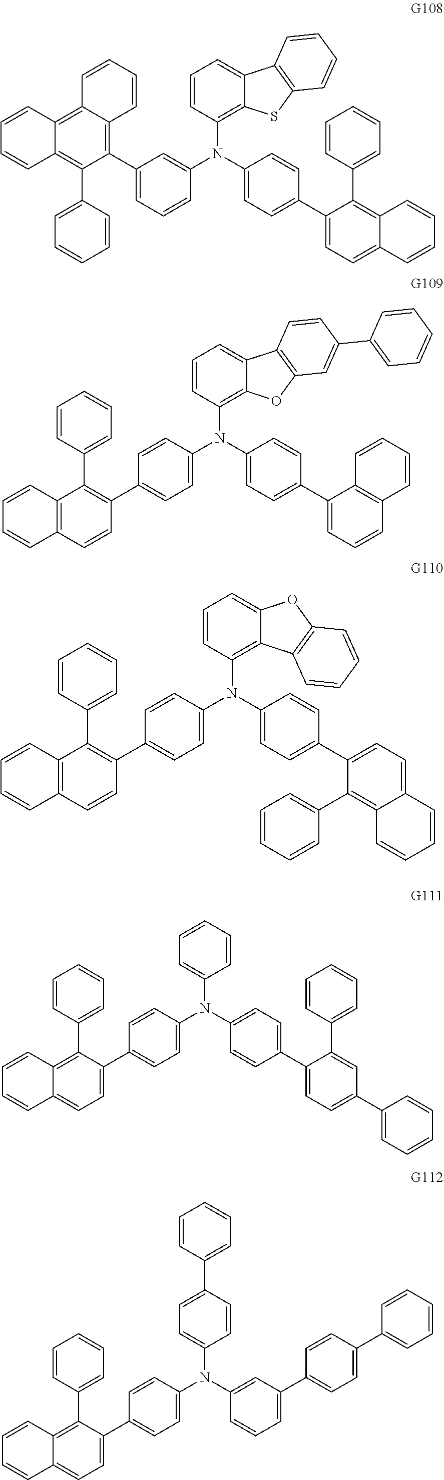 [SMILES: c1ccc(-c2c(-c3ccc(N(c4cccc(-c5c(-c6ccccc6)c6ccccc6c6ccccc56)c4)c4cccc5c4sc4ccccc45)cc3)ccc3ccccc23)cc1 ; c1ccc(-c2ccc3c(c2)oc2c(N(c4ccc(-c5ccc6ccccc6c5-c5ccccc5)cc4)c4ccc(-c5cccc6ccccc56)cc4)cccc23)cc1 ; c1ccc(-c2c(-c3ccc(N(c4ccc(-c5ccc6ccccc6c5-c5ccccc5)cc4)c4cccc5oc6ccccc6c45)cc3)ccc3ccccc23)cc1 ; c1ccc(-c2ccc(-c3ccc(N(c4ccccc4)c4ccc(-c5ccc6ccccc6c5-c5ccccc5)cc4)cc3)c(-c3ccccc3)c2)cc1 ; c1ccc(-c2ccc(-c3cccc(N(c4ccc(-c5ccccc5)cc4)c4ccc(-c5ccc6ccccc6c5-c5ccccc5)cc4)c3)cc2)cc1]

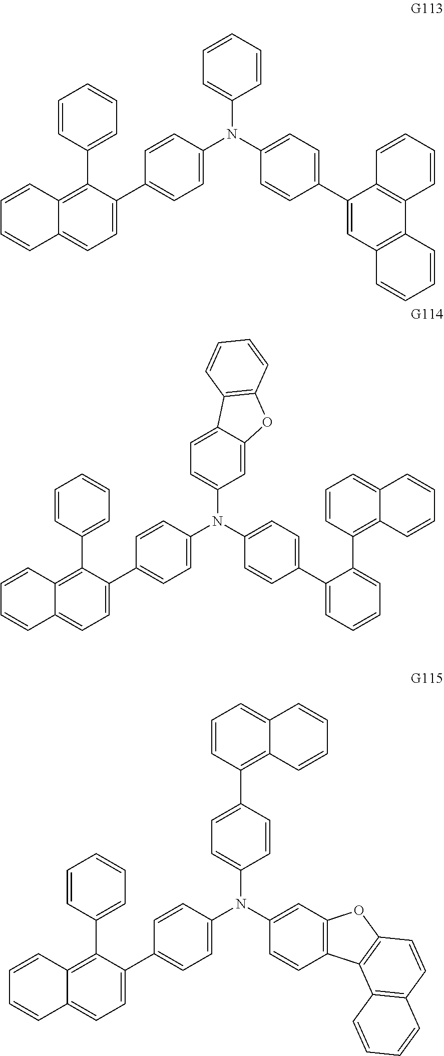 [SMILES: c1ccc(-c2c(-c3ccc(N(c4ccccc4)c4ccc(-c5cc6ccccc6c6ccccc56)cc4)cc3)ccc3ccccc23)cc1 ; c1ccc(-c2c(-c3ccc(N(c4ccc(-c5ccccc5-c5cccc6ccccc56)cc4)c4ccc5c(c4)oc4ccccc45)cc3)ccc3ccccc23)cc1 ; c1ccc(-c2c(-c3ccc(N(c4ccc(-c5cccc6ccccc56)cc4)c4ccc5c(c4)oc4ccc6ccccc6c45)cc3)ccc3ccccc23)cc1]

16. A monoamine compound represented by the following Formula 1: 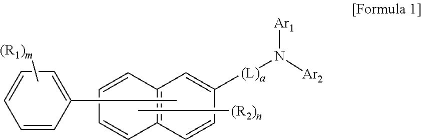
wherein in Formula 1,
Ar₁ and Ar₂ are each independently a substituted or unsubstituted alkyl group having 1 to 10 carbon atoms, a substituted or unsubstituted cycloalkyl group having 3 to 20 ring carbon atoms, a substituted or unsubstituted aryl group having 6 to 30 ring carbon atoms, or a substituted or unsubstituted heteroaryl group having 2 to 30 ring carbon atoms, provided that when any one of Ar₁ and Ar₂ is 3-dibenzofuranyl, the other one of Ar₁ and Ar₂ is not 9-phenanthryl,
L is a substituted or unsubstituted arylene group having 6 to 30 ring carbon atoms, or a substituted or unsubstituted heteroarylene group having 2 to 30 ring carbon atoms,
R₁ is a hydrogen atom, a deuterium atom, a halogen atom, a substituted or unsubstituted alkyl group having 1 to 20 carbon atoms, a substituted or unsubstituted cycloalkyl group having 3 to 20 ring carbon atoms, or a substituted or unsubstituted aryl group having 6 to 30 ring carbon atoms,
R₂ is a hydrogen atom, a deuterium atom, a halogen atom, a substituted or unsubstituted alkyl group having 1 to 20 carbon atoms, or a substituted or unsubstituted cycloalkyl group having 3 to 20 ring carbon atoms,
a is an integer of 0 to 3,
m is an integer of 0 to 1, and
n is an integer of 0 to 6.

17. The monoamine compound as claimed in claim 16, wherein Formula 1 is represented by any one of the following Formulae 2 to 8: 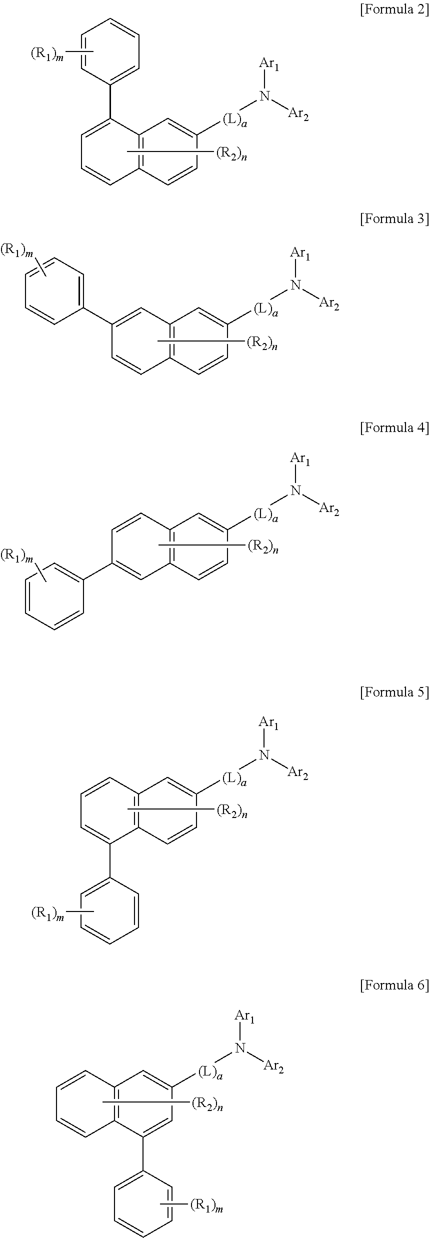 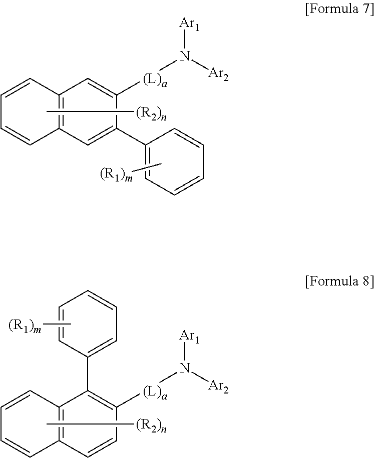
wherein in Formula 2 to 8,
Ar₁, Ar₂, L, R₁, R₂, a, m, and n are the same as defined in Formula 1.

18. The monoamine compound as claimed in claim 16, wherein L is a substituted or unsubstituted phenylene group.

19. The monoamine compound as claimed in claim 16, wherein Ar₁ and Ar₂ are each independently a substituted or unsubstituted aryl group having 6 to 12 ring carbon atoms.

20. The monoamine compound as claimed in claim 16, wherein Ar₁ and Ar₂ are each independently a substituted or unsubstituted heteroaryl group having 5 to 12 ring carbon atoms.

21. The monoamine compound as claimed in claim 16, wherein Ar₁ and Ar₂ are each independently a substituted or unsubstituted dibenzofuran group, a substituted or unsubstituted dibenzothiophene group, or a substituted or unsubstituted carbazole group.

22. The monoamine compound as claimed in claim 16, wherein at least one of Ar₁ and Ar₂ are a substituted or unsubstituted 4-dibenzofuran group.

23. The monoamine compound as claimed in claim 16, wherein Formula 1 is represented by the following Formula 9: 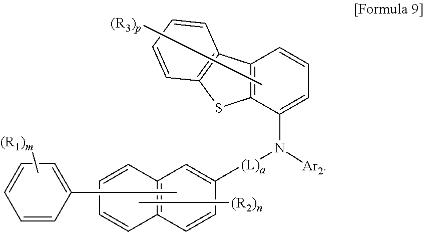
wherein in formula 9, R₃ may be a hydrogen atom, a deuterium atom, a halogen atom, a substituted or unsubstituted alkyl group having 1 to 20 carbon atoms, a substituted or unsubstituted cycloalkyl group having 3 to 20 ring carbon atoms, or a substituted or unsubstituted aryl group having 6 to 30 ring carbon atoms,
p may be an integer of 0 to 7, and
Ar₁, L, R₁, R₂, a, m, and n are the same as defined in Formula 1.

24. The monoamine compound as claimed in claim 16, wherein R₂ is a hydrogen atom or a deuterium atom.

25. The monoamine compound as claimed in claim 14, wherein the monoamine compound represented by Formula 1 is at least one selected from the group of compounds represented in the following Compound Groups 1 to 7: 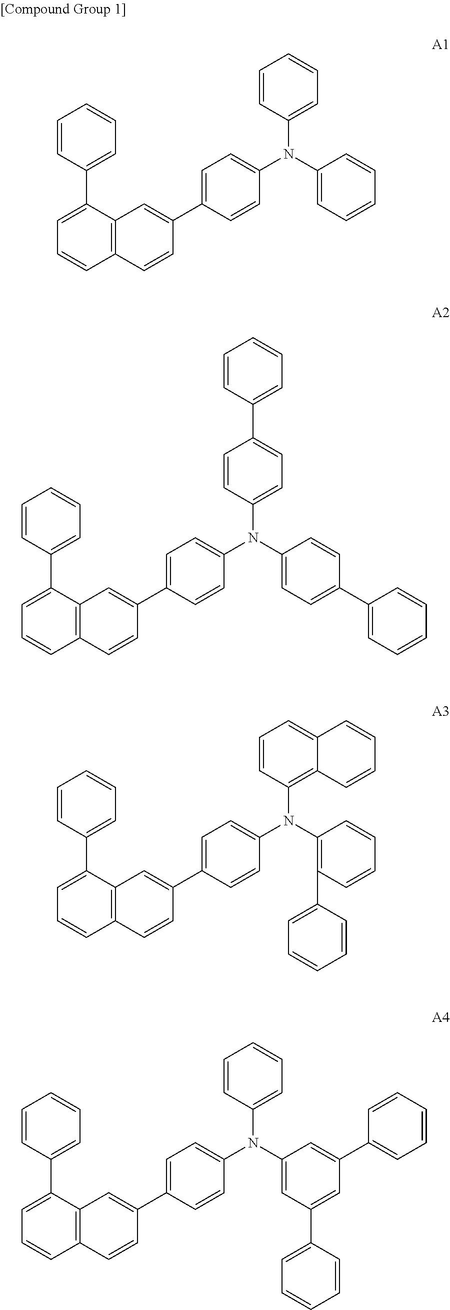 [SMILES: c1ccc(-c2cccc3ccc(-c4ccc(N(c5ccccc5)c5ccccc5)cc4)cc23)cc1 ; c1ccc(-c2ccc(N(c3ccc(-c4ccccc4)cc3)c3ccc(-c4ccc5cccc(-c6ccccc6)c5c4)cc3)cc2)cc1 ; c1ccc(-c2ccccc2N(c2ccc(-c3ccc4cccc(-c5ccccc5)c4c3)cc2)c2cccc3ccccc23)cc1 ; c1ccc(-c2cc(-c3ccccc3)cc(N(c3ccccc3)c3ccc(-c4ccc5cccc(-c6ccccc6)c5c4)cc3)c2)cc1] 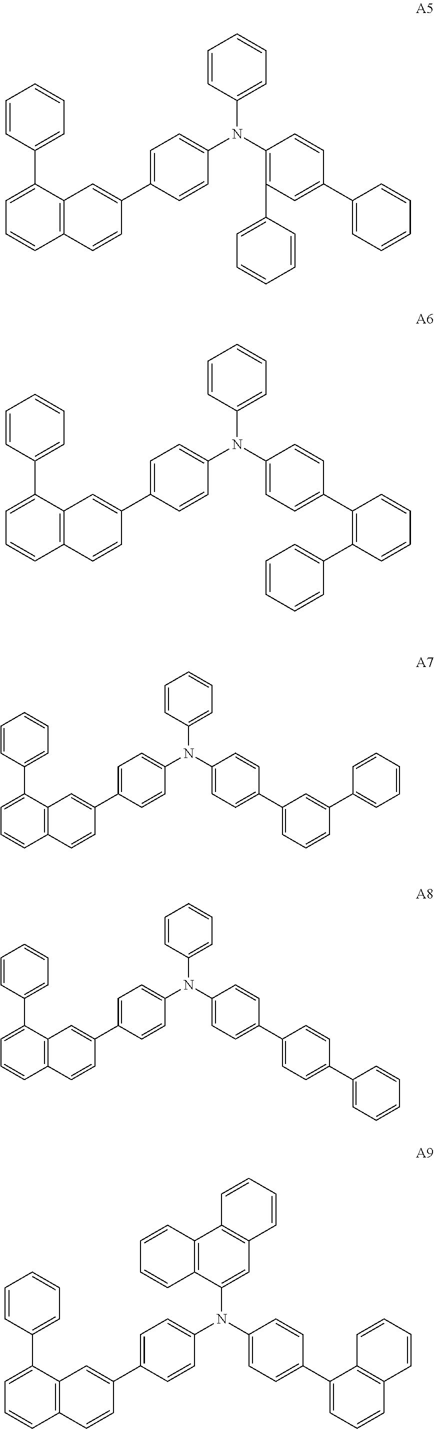 [SMILES: c1ccc(-c2ccc(N(c3ccccc3)c3ccc(-c4ccc5cccc(-c6ccccc6)c5c4)cc3)c(-c3ccccc3)c2)cc1 ; c1ccc(-c2ccccc2-c2ccc(N(c3ccccc3)c3ccc(-c4ccc5cccc(-c6ccccc6)c5c4)cc3)cc2)cc1 ; c1ccc(-c2cccc(-c3ccc(N(c4ccccc4)c4ccc(-c5ccc6cccc(-c7ccccc7)c6c5)cc4)cc3)c2)cc1 ; c1ccc(-c2ccc(-c3ccc(N(c4ccccc4)c4ccc(-c5ccc6cccc(-c7ccccc7)c6c5)cc4)cc3)cc2)cc1 ; c1ccc(-c2cccc3ccc(-c4ccc(N(c5ccc(-c6cccc7ccccc67)cc5)c5cc6ccccc6c6ccccc56)cc4)cc23)cc1] 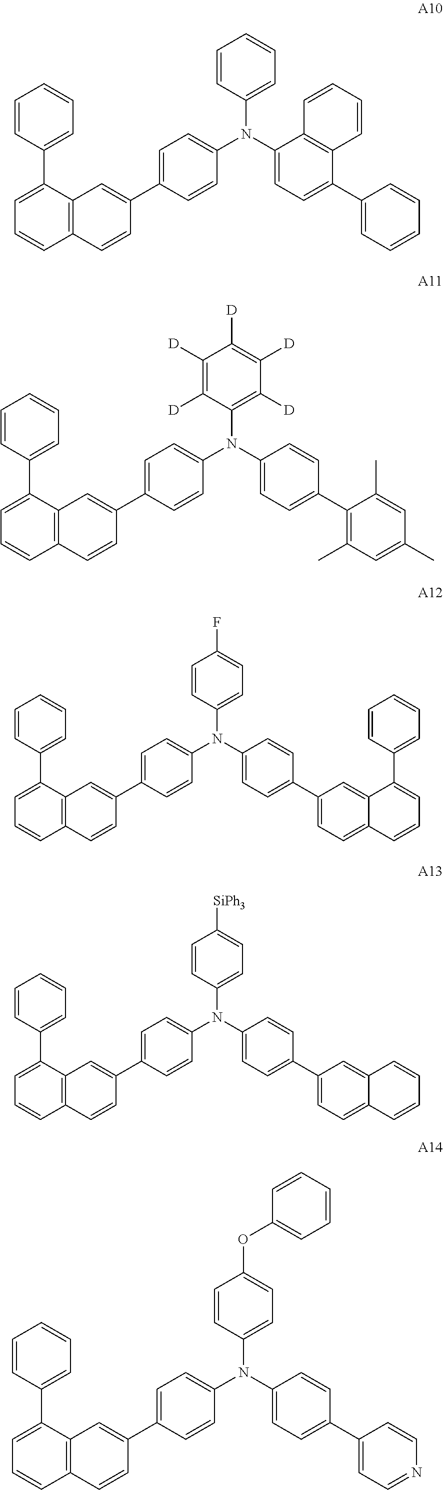 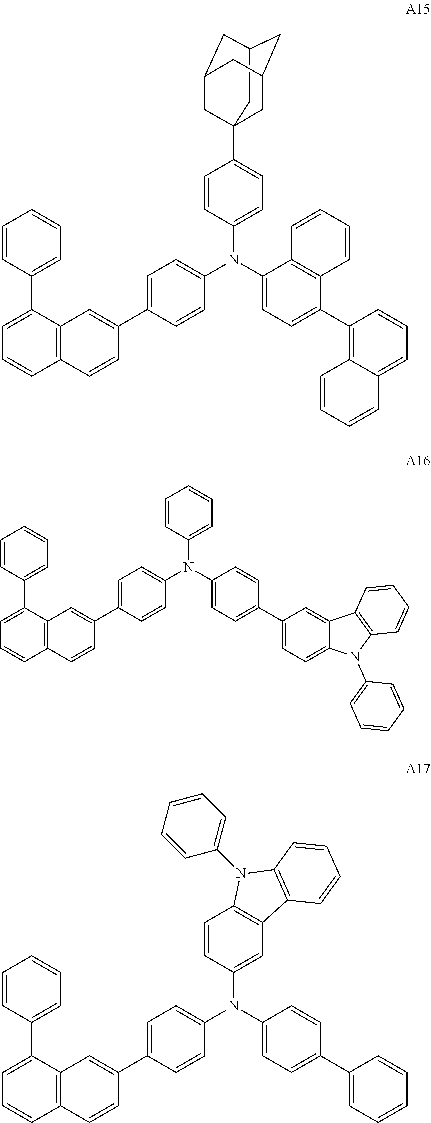 [SMILES: c1ccc(-c2cccc3ccc(-c4ccc(N(c5ccc(C67CC8CC(CC(C8)C6)C7)cc5)c5ccc(-c6cccc7ccccc67)c6ccccc56)cc4)cc23)cc1 ; c1ccc(-c2cccc3ccc(-c4ccc(N(c5ccccc5)c5ccc(-c6ccc7c(c6)c6ccccc6n7-c6ccccc6)cc5)cc4)cc23)cc1 ; c1ccc(-c2ccc(N(c3ccc(-c4ccc5cccc(-c6ccccc6)c5c4)cc3)c3ccc4c(c3)c3ccccc3n4-c3ccccc3)cc2)cc1] 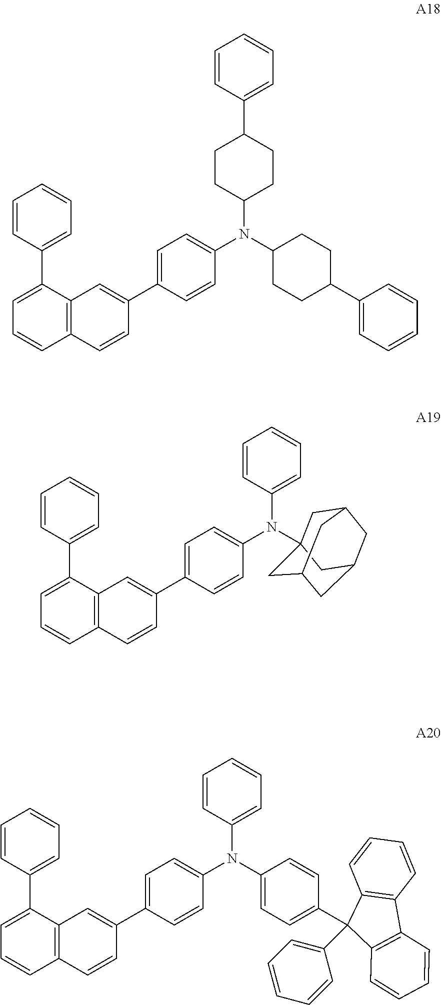 [SMILES: c1ccc(-c2cccc3ccc(-c4ccc(N(C5CCC(c6ccccc6)CC5)C5CCC(c6ccccc6)CC5)cc4)cc23)cc1 ; c1ccc(-c2cccc3ccc(-c4ccc(N(c5ccccc5)C56CC7CC(CC(C7)C5)C6)cc4)cc23)cc1 ; c1ccc(-c2cccc3ccc(-c4ccc(N(c5ccccc5)c5ccc(C6(c7ccccc7)c7ccccc7-c7ccccc76)cc5)cc4)cc23)cc1] 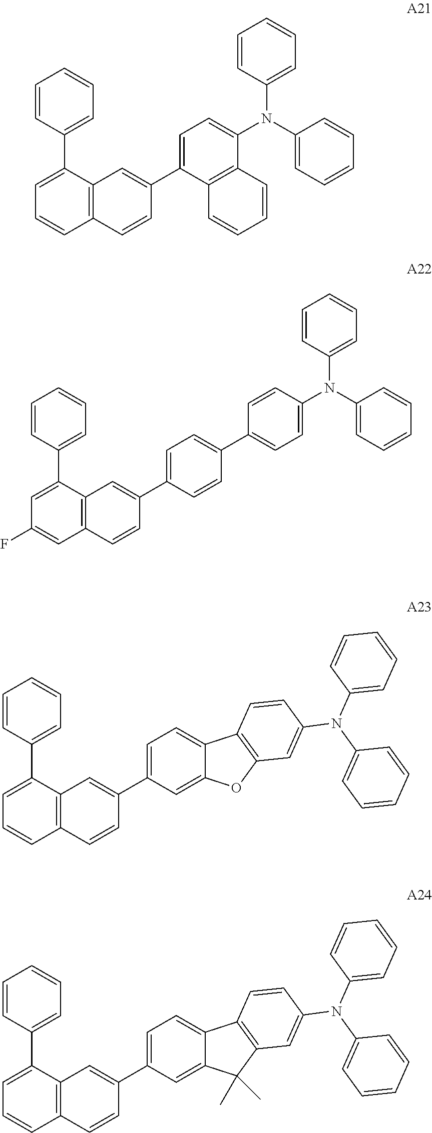 [SMILES: c1ccc(-c2cccc3ccc(-c4ccc(N(c5ccccc5)c5ccccc5)c5ccccc45)cc23)cc1 ; Fc1cc(-c2ccccc2)c2cc(-c3ccc(-c4ccc(N(c5ccccc5)c5ccccc5)cc4)cc3)ccc2c1 ; c1ccc(-c2cccc3ccc(-c4ccc5c(c4)oc4cc(N(c6ccccc6)c6ccccc6)ccc45)cc23)cc1 ; CC1(C)c2cc(-c3ccc4cccc(-c5ccccc5)c4c3)ccc2-c2ccc(N(c3ccccc3)c3ccccc3)cc21] 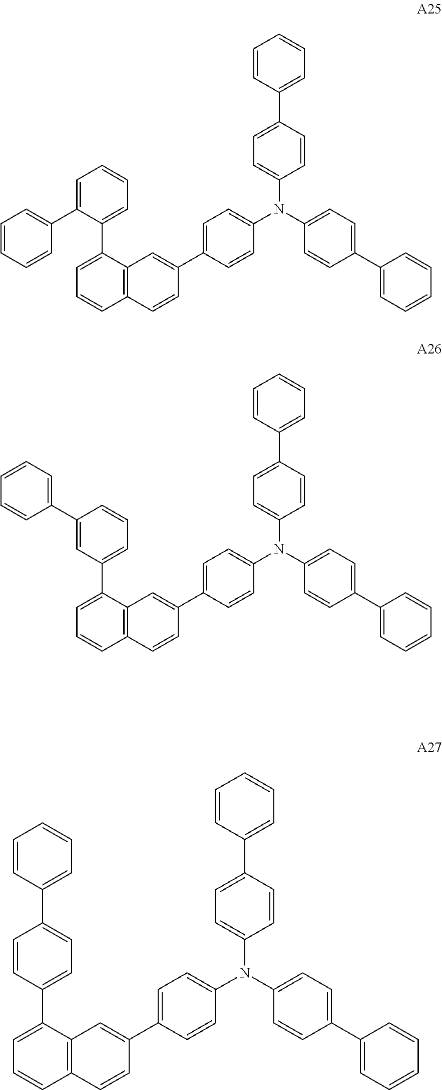 [SMILES: c1ccc(-c2ccc(N(c3ccc(-c4ccccc4)cc3)c3ccc(-c4ccc5cccc(-c6ccccc6-c6ccccc6)c5c4)cc3)cc2)cc1 ; c1ccc(-c2ccc(N(c3ccc(-c4ccccc4)cc3)c3ccc(-c4ccc5cccc(-c6cccc(-c7ccccc7)c6)c5c4)cc3)cc2)cc1 ; c1ccc(-c2ccc(-c3cccc4ccc(-c5ccc(N(c6ccc(-c7ccccc7)cc6)c6ccc(-c7ccccc7)cc6)cc5)cc34)cc2)cc1] 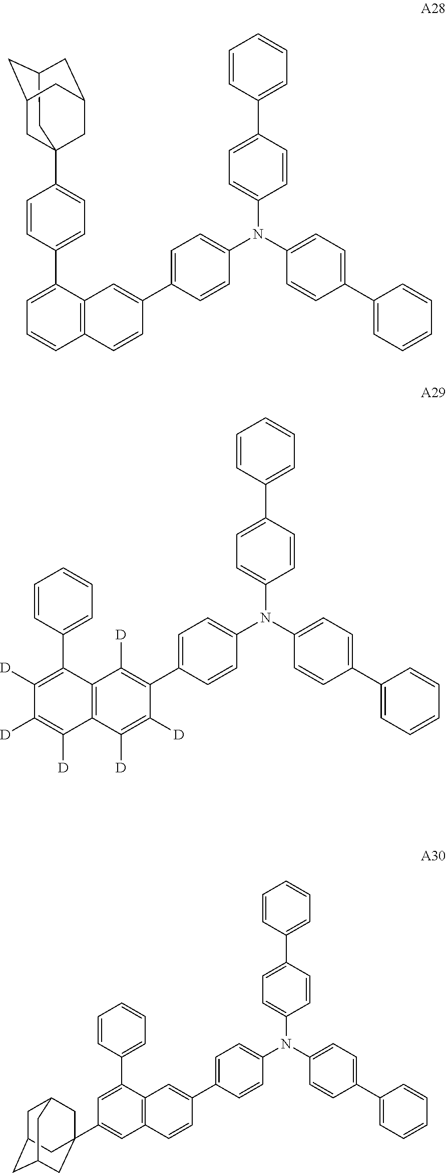 [SMILES: c1ccc(-c2ccc(N(c3ccc(-c4ccccc4)cc3)c3ccc(-c4ccc5cccc(-c6ccc(C78CC9CC(CC(C9)C7)C8)cc6)c5c4)cc3)cc2)cc1 ; [2H]c1c([2H])c(-c2ccccc2)c2c([2H])c(-c3ccc(N(c4ccc(-c5ccccc5)cc4)c4ccc(-c5ccccc5)cc4)cc3)c([2H])c([2H])c2c1[2H] ; c1ccc(-c2ccc(N(c3ccc(-c4ccccc4)cc3)c3ccc(-c4ccc5cc(C67CC8CC(CC(C8)C6)C7)cc(-c6ccccc6)c5c4)cc3)cc2)cc1] 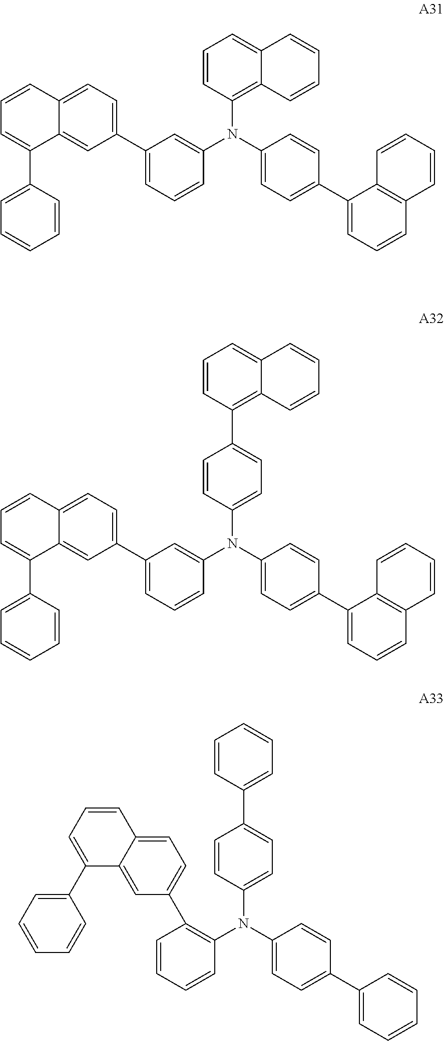 [SMILES: c1ccc(-c2cccc3ccc(-c4cccc(N(c5ccc(-c6cccc7ccccc67)cc5)c5cccc6ccccc56)c4)cc23)cc1 ; c1ccc(-c2cccc3ccc(-c4cccc(N(c5ccc(-c6cccc7ccccc67)cc5)c5ccc(-c6cccc7ccccc67)cc5)c4)cc23)cc1 ; c1ccc(-c2ccc(N(c3ccc(-c4ccccc4)cc3)c3ccccc3-c3ccc4cccc(-c5ccccc5)c4c3)cc2)cc1] 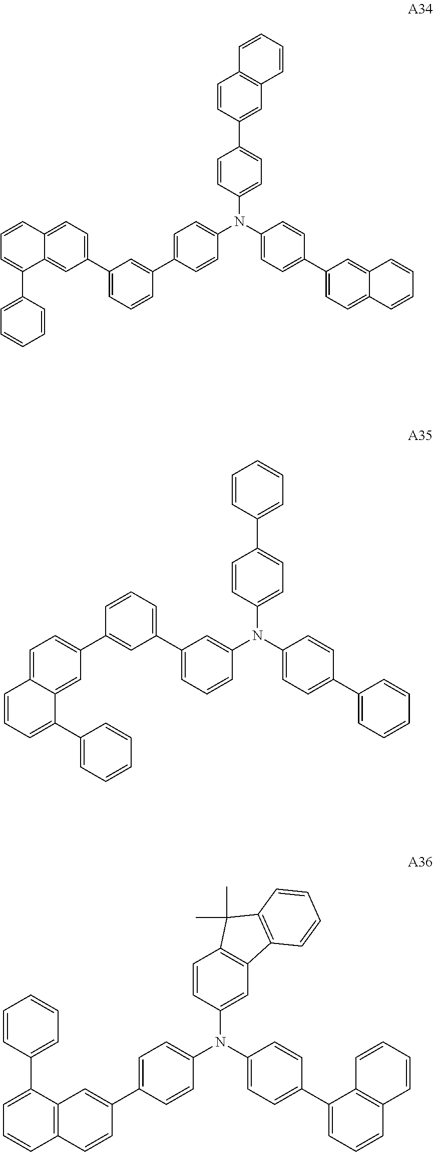 [SMILES: c1ccc(-c2cccc3ccc(-c4cccc(-c5ccc(N(c6ccc(-c7ccc8ccccc8c7)cc6)c6ccc(-c7ccc8ccccc8c7)cc6)cc5)c4)cc23)cc1 ; c1ccc(-c2ccc(N(c3ccc(-c4ccccc4)cc3)c3cccc(-c4cccc(-c5ccc6cccc(-c7ccccc7)c6c5)c4)c3)cc2)cc1 ; CC1(C)c2ccccc2-c2cc(N(c3ccc(-c4ccc5cccc(-c6ccccc6)c5c4)cc3)c3ccc(-c4cccc5ccccc45)cc3)ccc21] 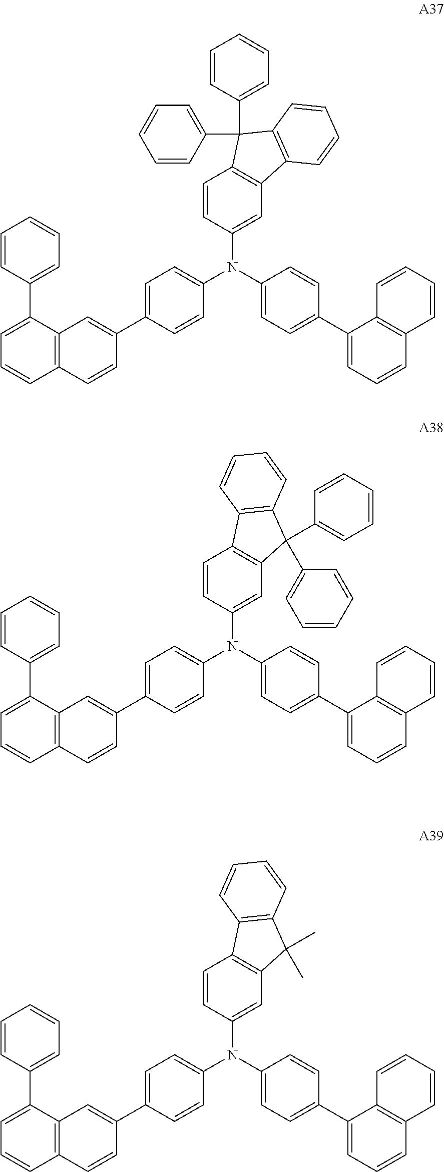 [SMILES: c1ccc(-c2cccc3ccc(-c4ccc(N(c5ccc(-c6cccc7ccccc67)cc5)c5ccc6c(c5)-c5ccccc5C6(c5ccccc5)c5ccccc5)cc4)cc23)cc1 ; c1ccc(-c2cccc3ccc(-c4ccc(N(c5ccc(-c6cccc7ccccc67)cc5)c5ccc6c(c5)C(c5ccccc5)(c5ccccc5)c5ccccc5-6)cc4)cc23)cc1 ; CC1(C)c2ccccc2-c2ccc(N(c3ccc(-c4ccc5cccc(-c6ccccc6)c5c4)cc3)c3ccc(-c4cccc5ccccc45)cc3)cc21] 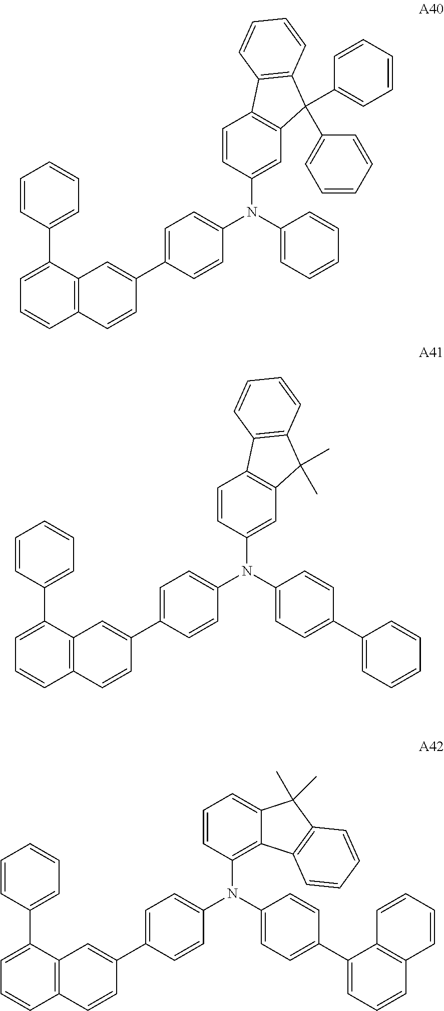 [SMILES: c1ccc(-c2cccc3ccc(-c4ccc(N(c5ccccc5)c5ccc6c(c5)C(c5ccccc5)(c5ccccc5)c5ccccc5-6)cc4)cc23)cc1 ; CC1(C)c2ccccc2-c2ccc(N(c3ccc(-c4ccccc4)cc3)c3ccc(-c4ccc5cccc(-c6ccccc6)c5c4)cc3)cc21 ; CC1(C)c2ccccc2-c2c(N(c3ccc(-c4ccc5cccc(-c6ccccc6)c5c4)cc3)c3ccc(-c4cccc5ccccc45)cc3)cccc21] 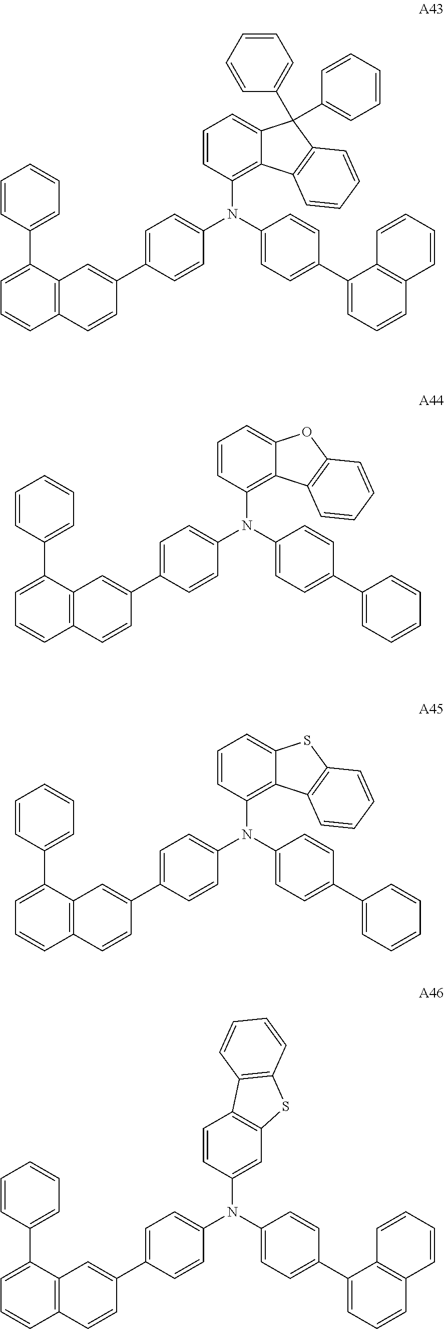 [SMILES: c1ccc(-c2cccc3ccc(-c4ccc(N(c5ccc(-c6cccc7ccccc67)cc5)c5cccc6c5-c5ccccc5C6(c5ccccc5)c5ccccc5)cc4)cc23)cc1 ; c1ccc(-c2ccc(N(c3ccc(-c4ccc5cccc(-c6ccccc6)c5c4)cc3)c3cccc4oc5ccccc5c34)cc2)cc1 ; c1ccc(-c2ccc(N(c3ccc(-c4ccc5cccc(-c6ccccc6)c5c4)cc3)c3cccc4sc5ccccc5c34)cc2)cc1 ; c1ccc(-c2cccc3ccc(-c4ccc(N(c5ccc(-c6cccc7ccccc67)cc5)c5ccc6c(c5)sc5ccccc56)cc4)cc23)cc1] 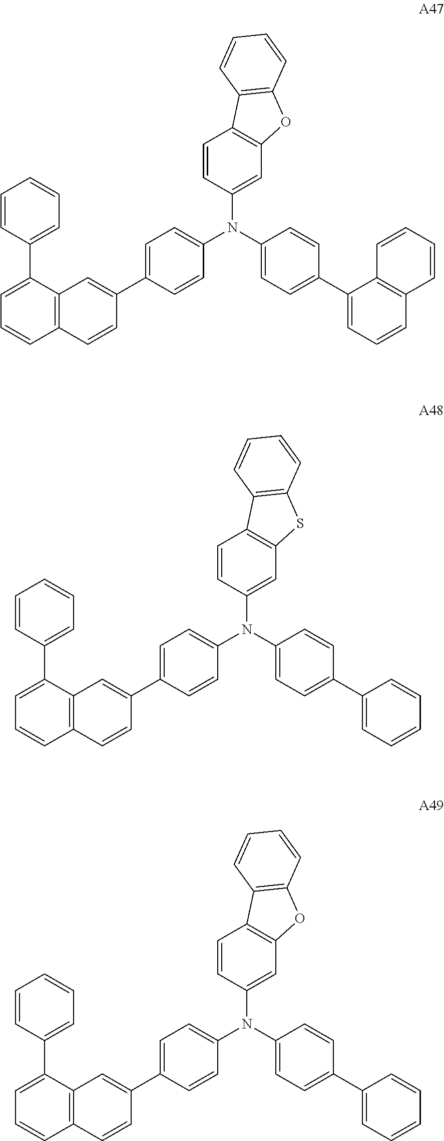 [SMILES: c1ccc(-c2cccc3ccc(-c4ccc(N(c5ccc(-c6cccc7ccccc67)cc5)c5ccc6c(c5)oc5ccccc56)cc4)cc23)cc1 ; c1ccc(-c2ccc(N(c3ccc(-c4ccc5cccc(-c6ccccc6)c5c4)cc3)c3ccc4c(c3)sc3ccccc34)cc2)cc1 ; c1ccc(-c2ccc(N(c3ccc(-c4ccc5cccc(-c6ccccc6)c5c4)cc3)c3ccc4c(c3)oc3ccccc34)cc2)cc1] 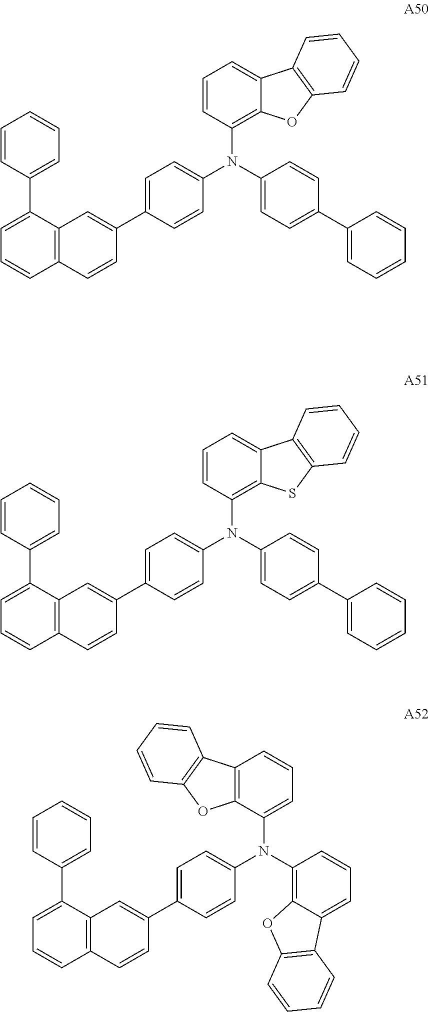 [SMILES: c1ccc(-c2ccc(N(c3ccc(-c4ccc5cccc(-c6ccccc6)c5c4)cc3)c3cccc4c3oc3ccccc34)cc2)cc1 ; c1ccc(-c2ccc(N(c3ccc(-c4ccc5cccc(-c6ccccc6)c5c4)cc3)c3cccc4c3sc3ccccc34)cc2)cc1 ; c1ccc(-c2cccc3ccc(-c4ccc(N(c5cccc6c5oc5ccccc56)c5cccc6c5oc5ccccc56)cc4)cc23)cc1] 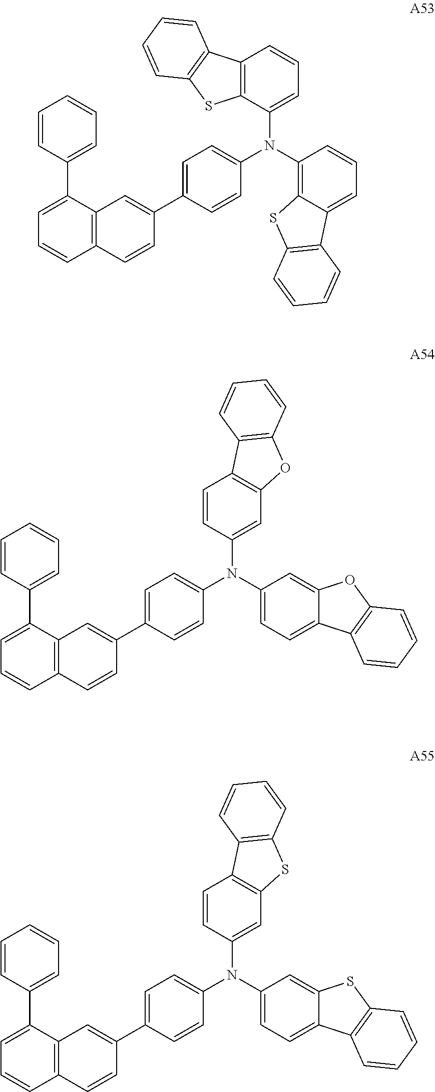 [SMILES: c1ccc(-c2cccc3ccc(-c4ccc(N(c5cccc6c5sc5ccccc56)c5cccc6c5sc5ccccc56)cc4)cc23)cc1 ; c1ccc(-c2cccc3ccc(-c4ccc(N(c5ccc6c(c5)oc5ccccc56)c5ccc6c(c5)oc5ccccc56)cc4)cc23)cc1 ; c1ccc(-c2cccc3ccc(-c4ccc(N(c5ccc6c(c5)sc5ccccc56)c5ccc6c(c5)sc5ccccc56)cc4)cc23)cc1] 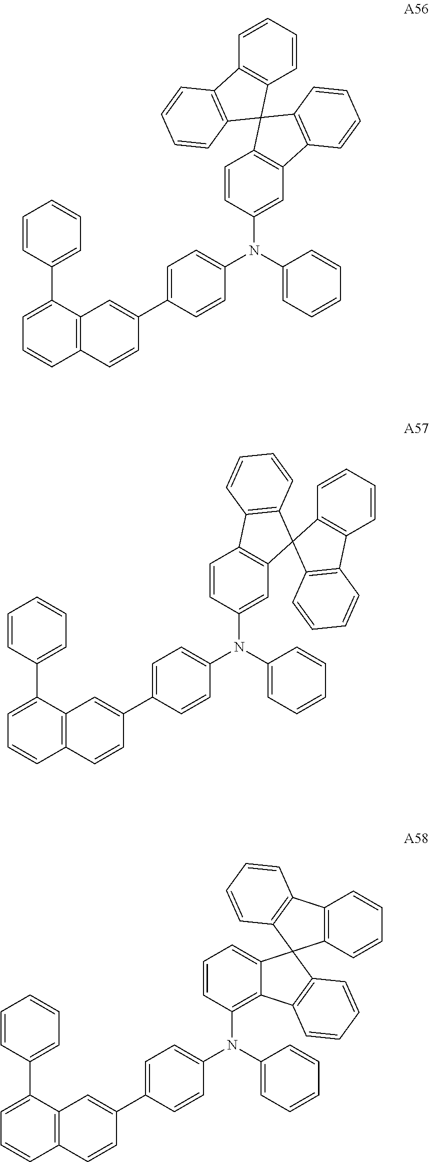 [SMILES: c1ccc(-c2cccc3ccc(-c4ccc(N(c5ccccc5)c5ccc6c(c5)-c5ccccc5C65c6ccccc6-c6ccccc65)cc4)cc23)cc1 ; c1ccc(-c2cccc3ccc(-c4ccc(N(c5ccccc5)c5ccc6c(c5)C5(c7ccccc7-c7ccccc75)c5ccccc5-6)cc4)cc23)cc1 ; c1ccc(-c2cccc3ccc(-c4ccc(N(c5ccccc5)c5cccc6c5-c5ccccc5C65c6ccccc6-c6ccccc65)cc4)cc23)cc1] 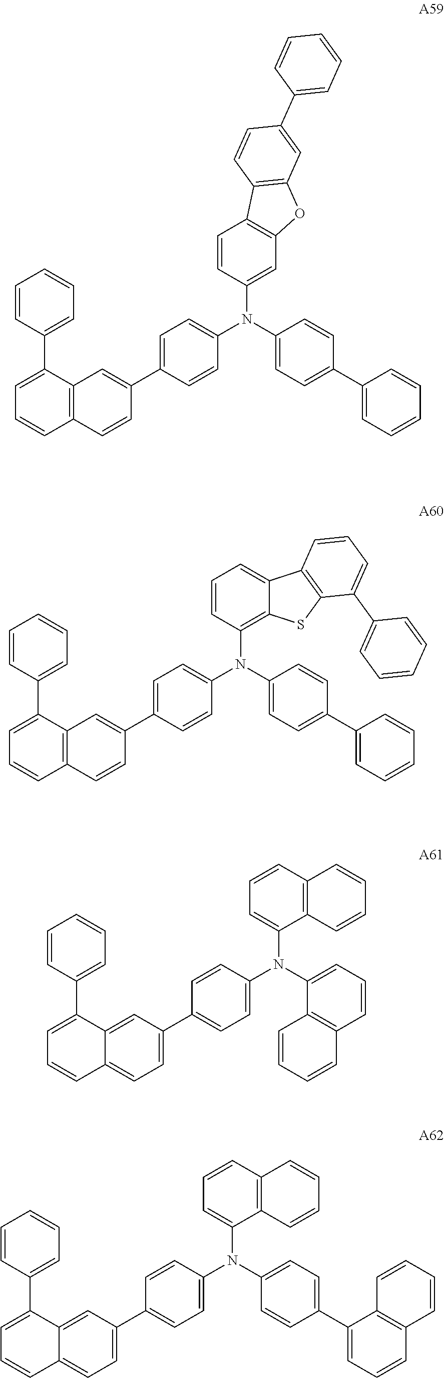 [SMILES: c1ccc(-c2ccc(N(c3ccc(-c4ccc5cccc(-c6ccccc6)c5c4)cc3)c3ccc4c(c3)oc3cc(-c5ccccc5)ccc34)cc2)cc1 ; c1ccc(-c2ccc(N(c3ccc(-c4ccc5cccc(-c6ccccc6)c5c4)cc3)c3cccc4c3sc3c(-c5ccccc5)cccc34)cc2)cc1 ; c1ccc(-c2cccc3ccc(-c4ccc(N(c5cccc6ccccc56)c5cccc6ccccc56)cc4)cc23)cc1 ; c1ccc(-c2cccc3ccc(-c4ccc(N(c5ccc(-c6cccc7ccccc67)cc5)c5cccc6ccccc56)cc4)cc23)cc1] 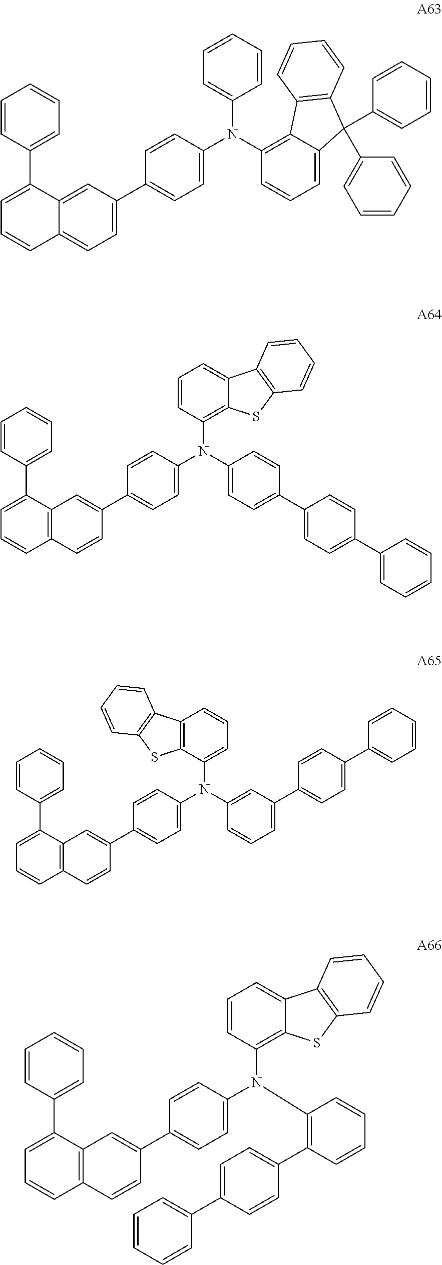 [SMILES: c1ccc(-c2cccc3ccc(-c4ccc(N(c5ccccc5)c5cccc6c5-c5ccccc5C6(c5ccccc5)c5ccccc5)cc4)cc23)cc1 ; c1ccc(-c2ccc(-c3ccc(N(c4ccc(-c5ccc6cccc(-c7ccccc7)c6c5)cc4)c4cccc5c4sc4ccccc45)cc3)cc2)cc1 ; c1ccc(-c2ccc(-c3cccc(N(c4ccc(-c5ccc6cccc(-c7ccccc7)c6c5)cc4)c4cccc5c4sc4ccccc45)c3)cc2)cc1 ; c1ccc(-c2ccc(-c3ccccc3N(c3ccc(-c4ccc5cccc(-c6ccccc6)c5c4)cc3)c3cccc4c3sc3ccccc34)cc2)cc1] 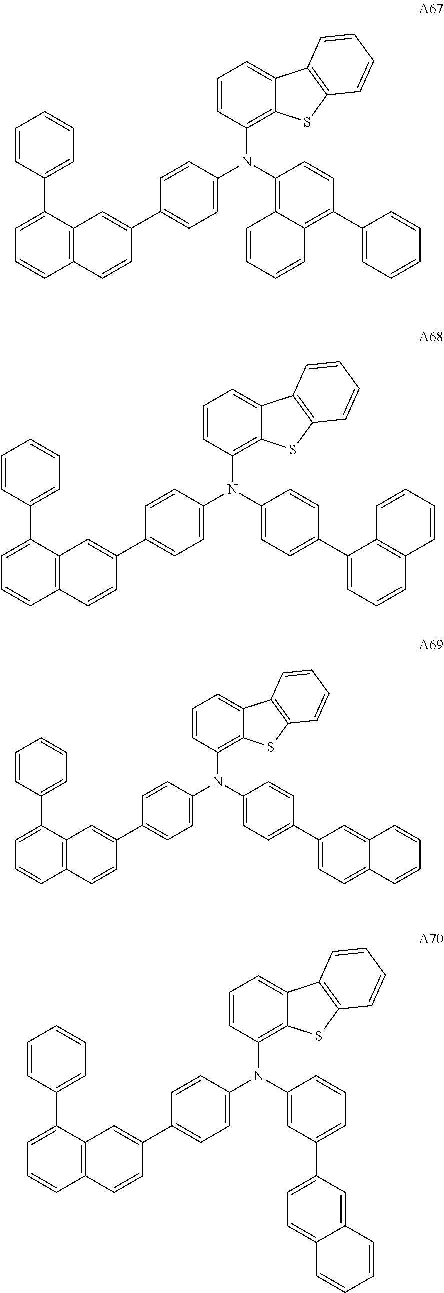 [SMILES: c1ccc(-c2cccc3ccc(-c4ccc(N(c5ccc(-c6ccccc6)c6ccccc56)c5cccc6c5sc5ccccc56)cc4)cc23)cc1 ; c1ccc(-c2cccc3ccc(-c4ccc(N(c5ccc(-c6cccc7ccccc67)cc5)c5cccc6c5sc5ccccc56)cc4)cc23)cc1 ; c1ccc(-c2cccc3ccc(-c4ccc(N(c5ccc(-c6ccc7ccccc7c6)cc5)c5cccc6c5sc5ccccc56)cc4)cc23)cc1 ; c1ccc(-c2cccc3ccc(-c4ccc(N(c5cccc(-c6ccc7ccccc7c6)c5)c5cccc6c5sc5ccccc56)cc4)cc23)cc1] 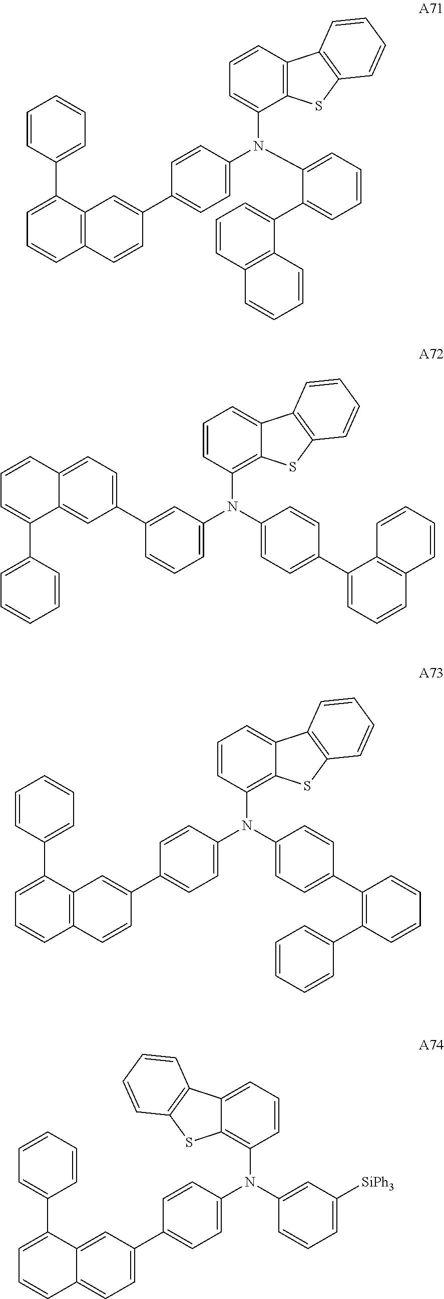 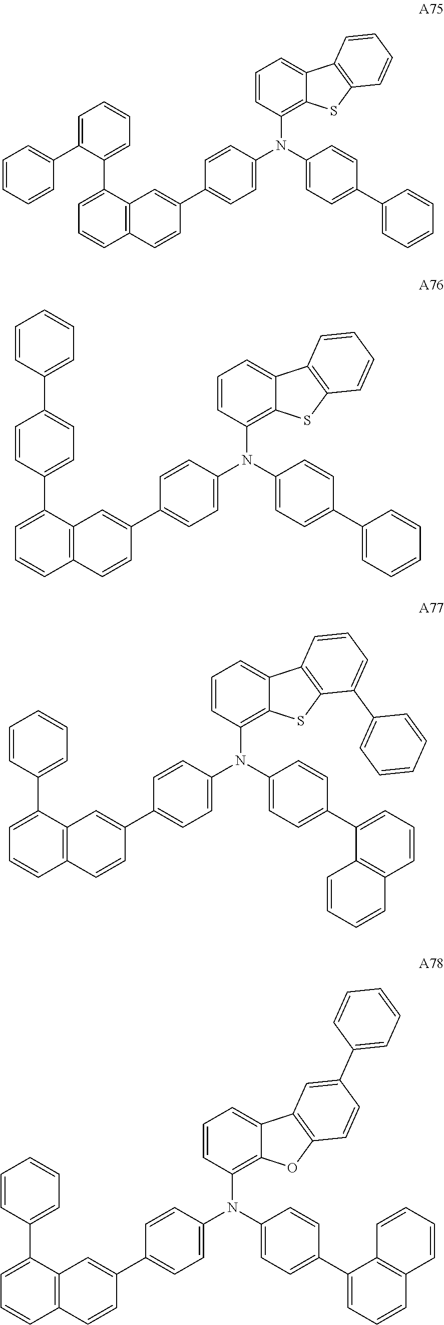 [SMILES: c1ccc(-c2ccc(N(c3ccc(-c4ccc5cccc(-c6ccccc6-c6ccccc6)c5c4)cc3)c3cccc4c3sc3ccccc34)cc2)cc1 ; c1ccc(-c2ccc(-c3cccc4ccc(-c5ccc(N(c6ccc(-c7ccccc7)cc6)c6cccc7c6sc6ccccc67)cc5)cc34)cc2)cc1 ; c1ccc(-c2cccc3ccc(-c4ccc(N(c5ccc(-c6cccc7ccccc67)cc5)c5cccc6c5sc5c(-c7ccccc7)cccc56)cc4)cc23)cc1 ; c1ccc(-c2ccc3oc4c(N(c5ccc(-c6ccc7cccc(-c8ccccc8)c7c6)cc5)c5ccc(-c6cccc7ccccc67)cc5)cccc4c3c2)cc1] 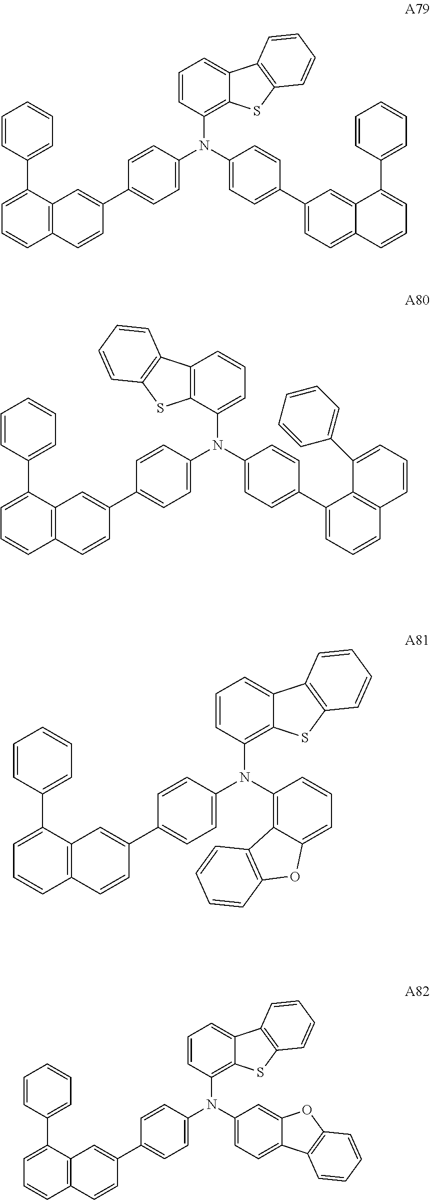 [SMILES: c1ccc(-c2cccc3ccc(-c4ccc(N(c5ccc(-c6ccc7cccc(-c8ccccc8)c7c6)cc5)c5cccc6c5sc5ccccc56)cc4)cc23)cc1 ; c1ccc(-c2cccc3ccc(-c4ccc(N(c5ccc(-c6cccc7cccc(-c8ccccc8)c67)cc5)c5cccc6c5sc5ccccc56)cc4)cc23)cc1 ; c1ccc(-c2cccc3ccc(-c4ccc(N(c5cccc6c5sc5ccccc56)c5cccc6oc7ccccc7c56)cc4)cc23)cc1 ; c1ccc(-c2cccc3ccc(-c4ccc(N(c5ccc6c(c5)oc5ccccc56)c5cccc6c5sc5ccccc56)cc4)cc23)cc1] 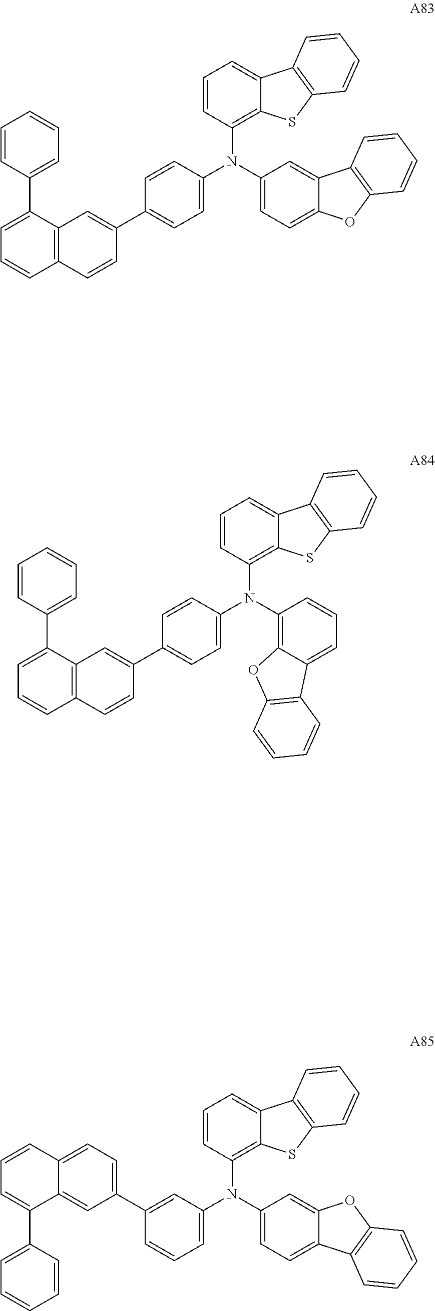 [SMILES: c1ccc(-c2cccc3ccc(-c4ccc(N(c5ccc6oc7ccccc7c6c5)c5cccc6c5sc5ccccc56)cc4)cc23)cc1 ; c1ccc(-c2cccc3ccc(-c4ccc(N(c5cccc6c5oc5ccccc56)c5cccc6c5sc5ccccc56)cc4)cc23)cc1 ; c1ccc(-c2cccc3ccc(-c4cccc(N(c5ccc6c(c5)oc5ccccc56)c5cccc6c5sc5ccccc56)c4)cc23)cc1] 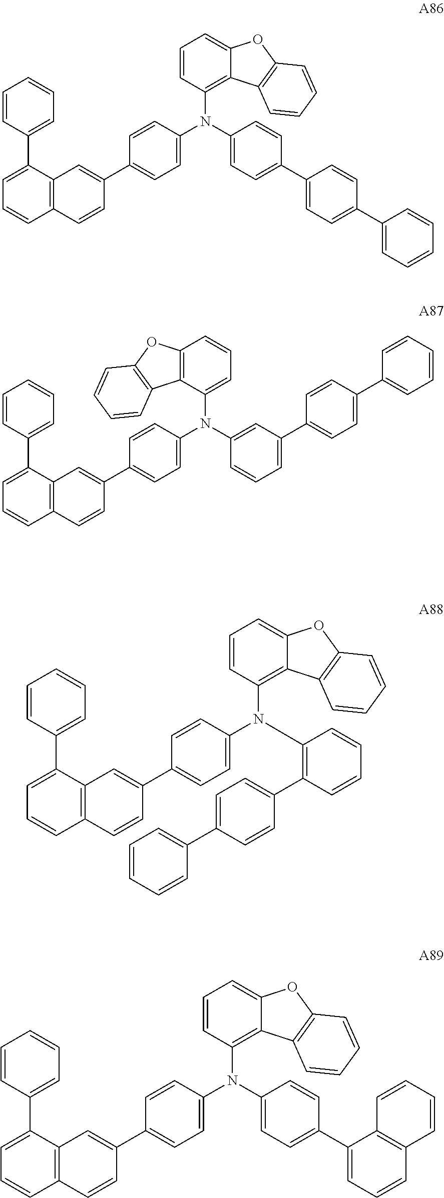 [SMILES: c1ccc(-c2ccc(-c3ccc(N(c4ccc(-c5ccc6cccc(-c7ccccc7)c6c5)cc4)c4cccc5oc6ccccc6c45)cc3)cc2)cc1 ; c1ccc(-c2ccc(-c3cccc(N(c4ccc(-c5ccc6cccc(-c7ccccc7)c6c5)cc4)c4cccc5oc6ccccc6c45)c3)cc2)cc1 ; c1ccc(-c2ccc(-c3ccccc3N(c3ccc(-c4ccc5cccc(-c6ccccc6)c5c4)cc3)c3cccc4oc5ccccc5c34)cc2)cc1 ; c1ccc(-c2cccc3ccc(-c4ccc(N(c5ccc(-c6cccc7ccccc67)cc5)c5cccc6oc7ccccc7c56)cc4)cc23)cc1] 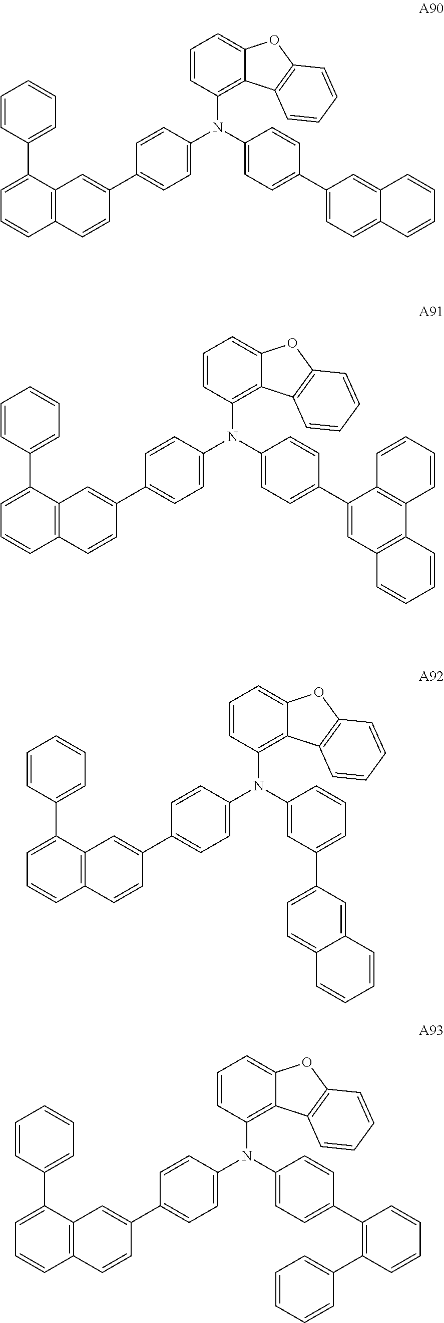 [SMILES: c1ccc(-c2cccc3ccc(-c4ccc(N(c5ccc(-c6ccc7ccccc7c6)cc5)c5cccc6oc7ccccc7c56)cc4)cc23)cc1 ; c1ccc(-c2cccc3ccc(-c4ccc(N(c5ccc(-c6cc7ccccc7c7ccccc67)cc5)c5cccc6oc7ccccc7c56)cc4)cc23)cc1 ; c1ccc(-c2cccc3ccc(-c4ccc(N(c5cccc(-c6ccc7ccccc7c6)c5)c5cccc6oc7ccccc7c56)cc4)cc23)cc1 ; c1ccc(-c2ccccc2-c2ccc(N(c3ccc(-c4ccc5cccc(-c6ccccc6)c5c4)cc3)c3cccc4oc5ccccc5c34)cc2)cc1] 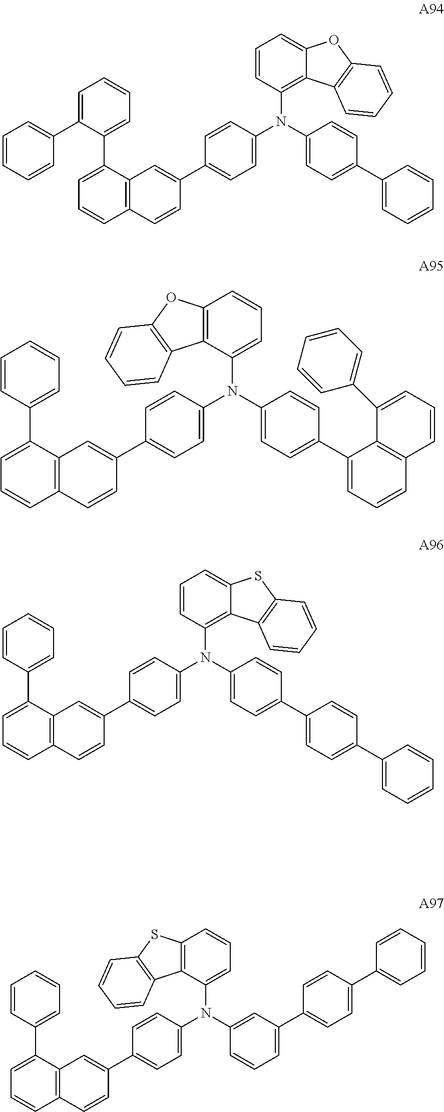 [SMILES: c1ccc(-c2ccc(N(c3ccc(-c4ccc5cccc(-c6ccccc6-c6ccccc6)c5c4)cc3)c3cccc4oc5ccccc5c34)cc2)cc1 ; c1ccc(-c2cccc3ccc(-c4ccc(N(c5ccc(-c6cccc7cccc(-c8ccccc8)c67)cc5)c5cccc6oc7ccccc7c56)cc4)cc23)cc1 ; c1ccc(-c2ccc(-c3ccc(N(c4ccc(-c5ccc6cccc(-c7ccccc7)c6c5)cc4)c4cccc5sc6ccccc6c45)cc3)cc2)cc1 ; c1ccc(-c2ccc(-c3cccc(N(c4ccc(-c5ccc6cccc(-c7ccccc7)c6c5)cc4)c4cccc5sc6ccccc6c45)c3)cc2)cc1] 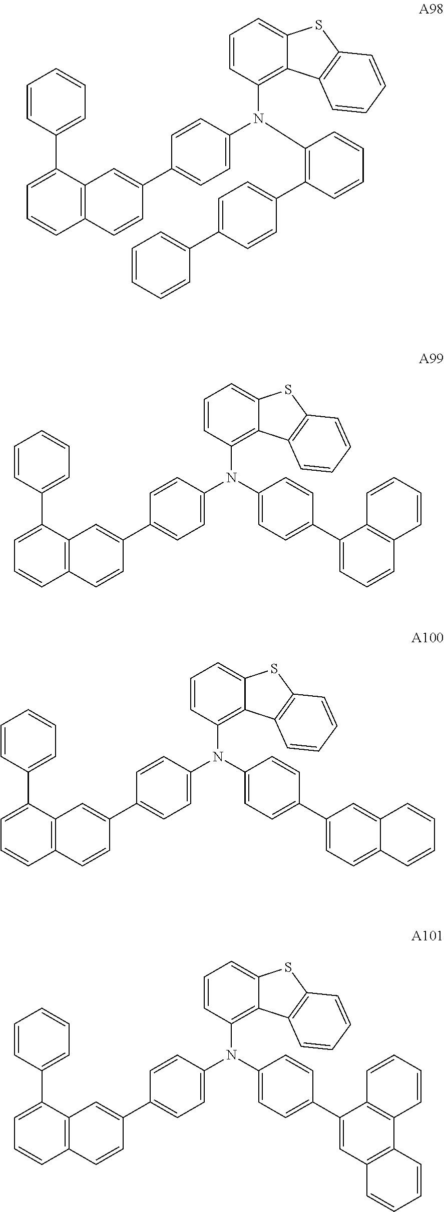 [SMILES: c1ccc(-c2ccc(-c3ccccc3N(c3ccc(-c4ccc5cccc(-c6ccccc6)c5c4)cc3)c3cccc4sc5ccccc5c34)cc2)cc1 ; c1ccc(-c2cccc3ccc(-c4ccc(N(c5ccc(-c6cccc7ccccc67)cc5)c5cccc6sc7ccccc7c56)cc4)cc23)cc1 ; c1ccc(-c2cccc3ccc(-c4ccc(N(c5ccc(-c6ccc7ccccc7c6)cc5)c5cccc6sc7ccccc7c56)cc4)cc23)cc1 ; c1ccc(-c2cccc3ccc(-c4ccc(N(c5ccc(-c6cc7ccccc7c7ccccc67)cc5)c5cccc6sc7ccccc7c56)cc4)cc23)cc1] 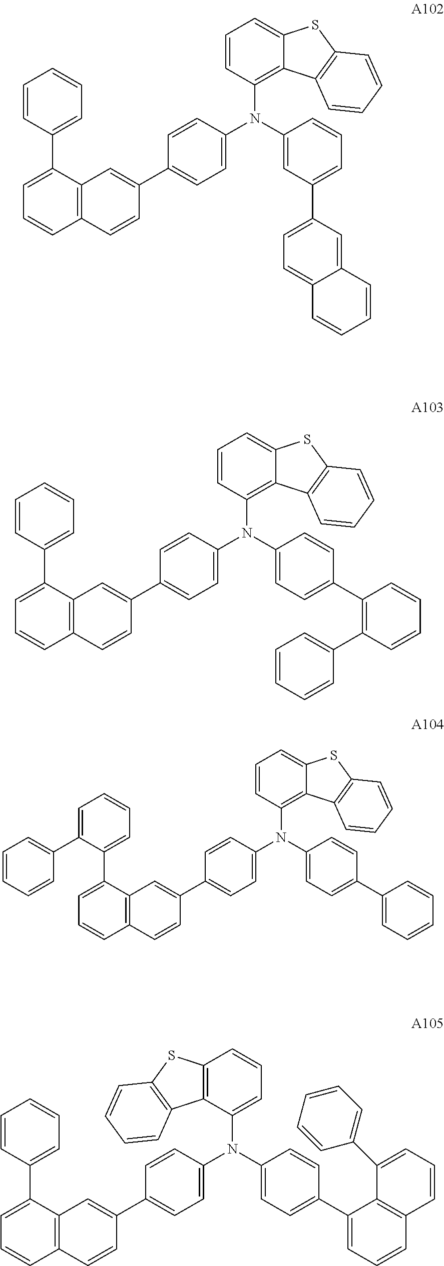 [SMILES: c1ccc(-c2cccc3ccc(-c4ccc(N(c5cccc(-c6ccc7ccccc7c6)c5)c5cccc6sc7ccccc7c56)cc4)cc23)cc1 ; c1ccc(-c2ccccc2-c2ccc(N(c3ccc(-c4ccc5cccc(-c6ccccc6)c5c4)cc3)c3cccc4sc5ccccc5c34)cc2)cc1 ; c1ccc(-c2ccc(N(c3ccc(-c4ccc5cccc(-c6ccccc6-c6ccccc6)c5c4)cc3)c3cccc4sc5ccccc5c34)cc2)cc1 ; c1ccc(-c2cccc3ccc(-c4ccc(N(c5ccc(-c6cccc7cccc(-c8ccccc8)c67)cc5)c5cccc6sc7ccccc7c56)cc4)cc23)cc1] 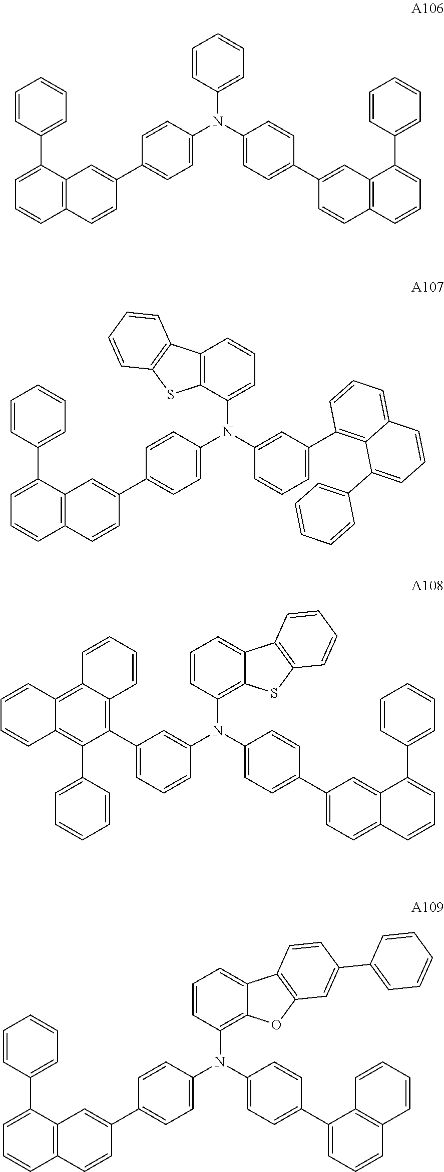 [SMILES: c1ccc(-c2cccc3ccc(-c4ccc(N(c5ccccc5)c5ccc(-c6ccc7cccc(-c8ccccc8)c7c6)cc5)cc4)cc23)cc1 ; c1ccc(-c2cccc3ccc(-c4ccc(N(c5cccc(-c6cccc7cccc(-c8ccccc8)c67)c5)c5cccc6c5sc5ccccc56)cc4)cc23)cc1 ; c1ccc(-c2cccc3ccc(-c4ccc(N(c5cccc(-c6c(-c7ccccc7)c7ccccc7c7ccccc67)c5)c5cccc6c5sc5ccccc56)cc4)cc23)cc1 ; c1ccc(-c2ccc3c(c2)oc2c(N(c4ccc(-c5ccc6cccc(-c7ccccc7)c6c5)cc4)c4ccc(-c5cccc6ccccc56)cc4)cccc23)cc1] 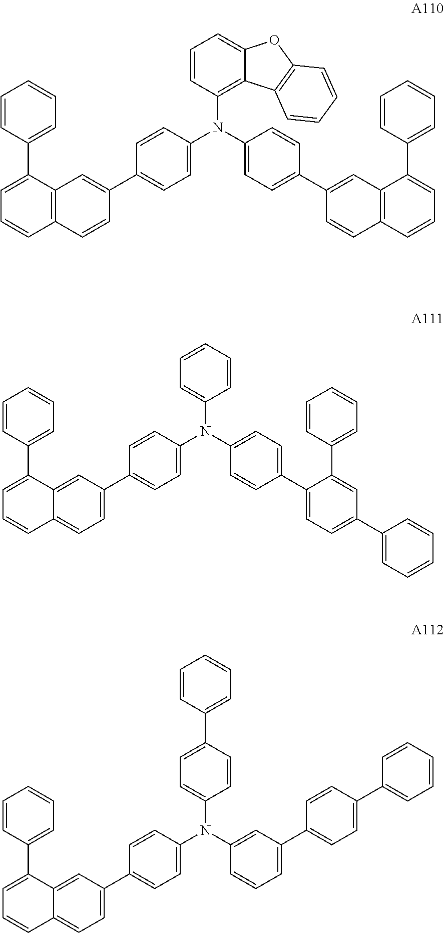 [SMILES: c1ccc(-c2cccc3ccc(-c4ccc(N(c5ccc(-c6ccc7cccc(-c8ccccc8)c7c6)cc5)c5cccc6oc7ccccc7c56)cc4)cc23)cc1 ; c1ccc(-c2ccc(-c3ccc(N(c4ccccc4)c4ccc(-c5ccc6cccc(-c7ccccc7)c6c5)cc4)cc3)c(-c3ccccc3)c2)cc1 ; c1ccc(-c2ccc(-c3cccc(N(c4ccc(-c5ccccc5)cc4)c4ccc(-c5ccc6cccc(-c7ccccc7)c6c5)cc4)c3)cc2)cc1] 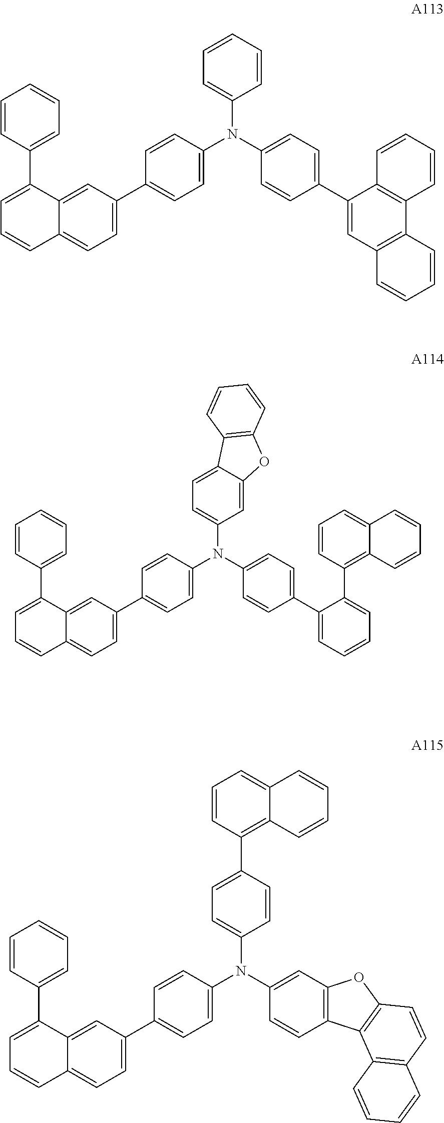 [SMILES: c1ccc(-c2cccc3ccc(-c4ccc(N(c5ccccc5)c5ccc(-c6cc7ccccc7c7ccccc67)cc5)cc4)cc23)cc1 ; c1ccc(-c2cccc3ccc(-c4ccc(N(c5ccc(-c6ccccc6-c6cccc7ccccc67)cc5)c5ccc6c(c5)oc5ccccc56)cc4)cc23)cc1 ; c1ccc(-c2cccc3ccc(-c4ccc(N(c5ccc(-c6cccc7ccccc67)cc5)c5ccc6c(c5)oc5ccc7ccccc7c56)cc4)cc23)cc1] 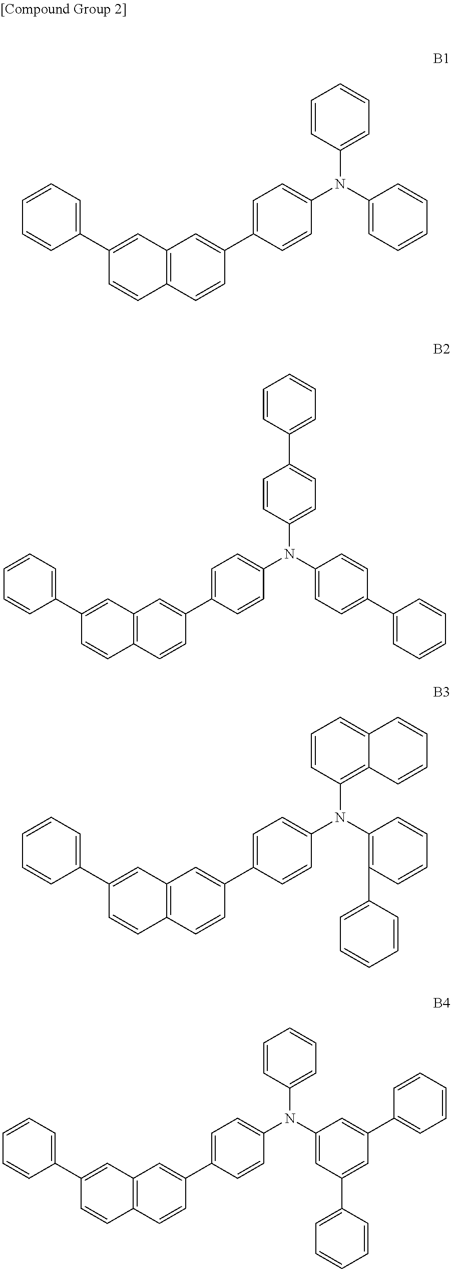 [SMILES: c1ccc(-c2ccc3ccc(-c4ccc(N(c5ccccc5)c5ccccc5)cc4)cc3c2)cc1 ; c1ccc(-c2ccc(N(c3ccc(-c4ccccc4)cc3)c3ccc(-c4ccc5ccc(-c6ccccc6)cc5c4)cc3)cc2)cc1 ; c1ccc(-c2ccc3ccc(-c4ccc(N(c5ccccc5-c5ccccc5)c5cccc6ccccc56)cc4)cc3c2)cc1 ; c1ccc(-c2cc(-c3ccccc3)cc(N(c3ccccc3)c3ccc(-c4ccc5ccc(-c6ccccc6)cc5c4)cc3)c2)cc1] 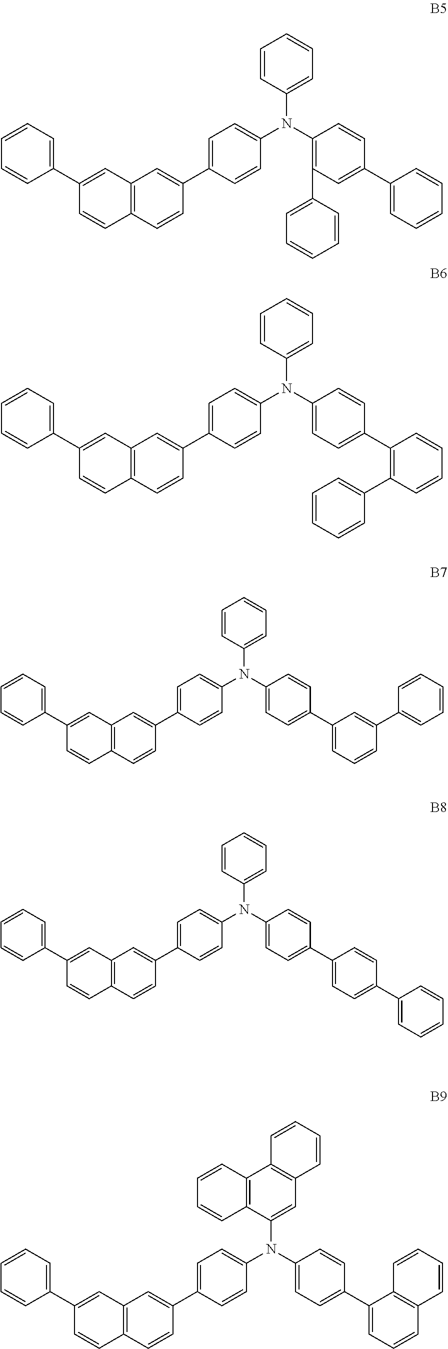 [SMILES: c1ccc(-c2ccc(N(c3ccccc3)c3ccc(-c4ccc5ccc(-c6ccccc6)cc5c4)cc3)c(-c3ccccc3)c2)cc1 ; c1ccc(-c2ccc3ccc(-c4ccc(N(c5ccccc5)c5ccc(-c6ccccc6-c6ccccc6)cc5)cc4)cc3c2)cc1 ; c1ccc(-c2cccc(-c3ccc(N(c4ccccc4)c4ccc(-c5ccc6ccc(-c7ccccc7)cc6c5)cc4)cc3)c2)cc1 ; c1ccc(-c2ccc(-c3ccc(N(c4ccccc4)c4ccc(-c5ccc6ccc(-c7ccccc7)cc6c5)cc4)cc3)cc2)cc1 ; c1ccc(-c2ccc3ccc(-c4ccc(N(c5ccc(-c6cccc7ccccc67)cc5)c5cc6ccccc6c6ccccc56)cc4)cc3c2)cc1] 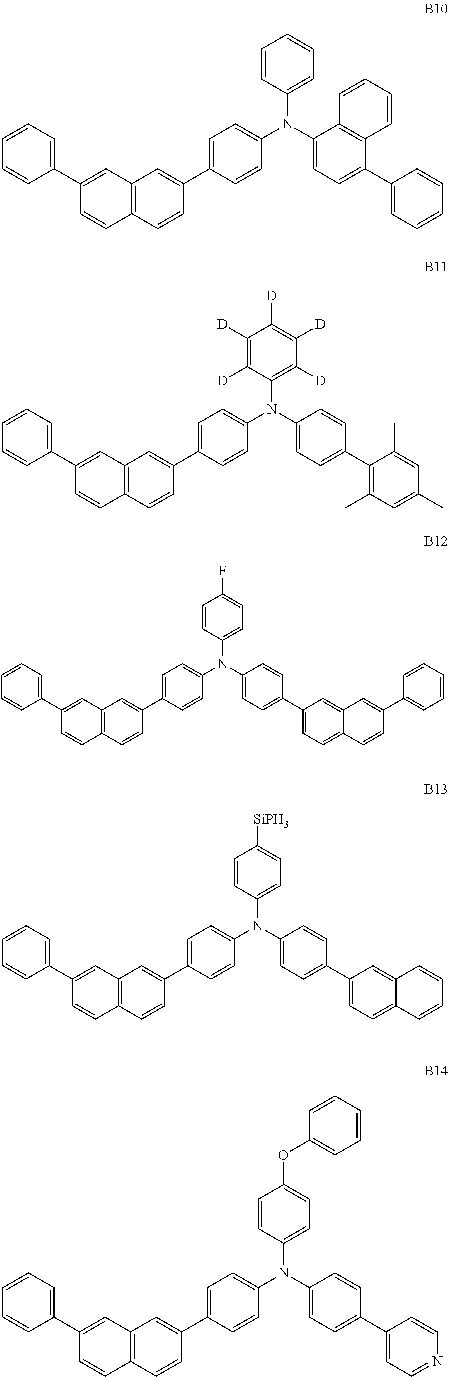 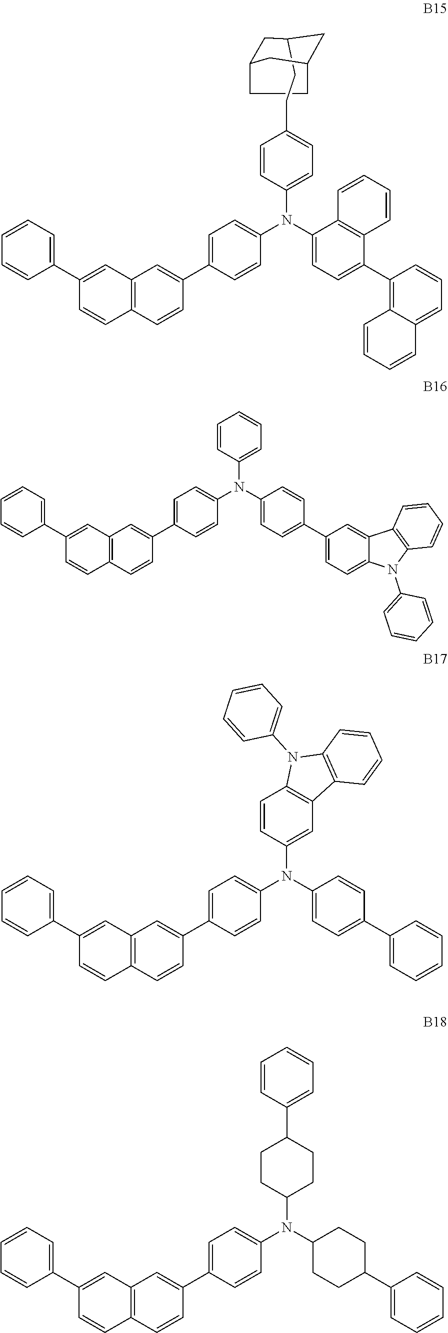 [SMILES: c1ccc(-c2ccc3ccc(-c4ccc(N(c5ccc(CCC6CC7CCC(C6)C7)cc5)c5ccc(-c6cccc7ccccc67)c6ccccc56)cc4)cc3c2)cc1 ; c1ccc(-c2ccc3ccc(-c4ccc(N(c5ccccc5)c5ccc(-c6ccc7c(c6)c6ccccc6n7-c6ccccc6)cc5)cc4)cc3c2)cc1 ; c1ccc(-c2ccc(N(c3ccc(-c4ccc5ccc(-c6ccccc6)cc5c4)cc3)c3ccc4c(c3)c3ccccc3n4-c3ccccc3)cc2)cc1 ; c1ccc(-c2ccc3ccc(-c4ccc(N(C5CCC(c6ccccc6)CC5)C5CCC(c6ccccc6)CC5)cc4)cc3c2)cc1] 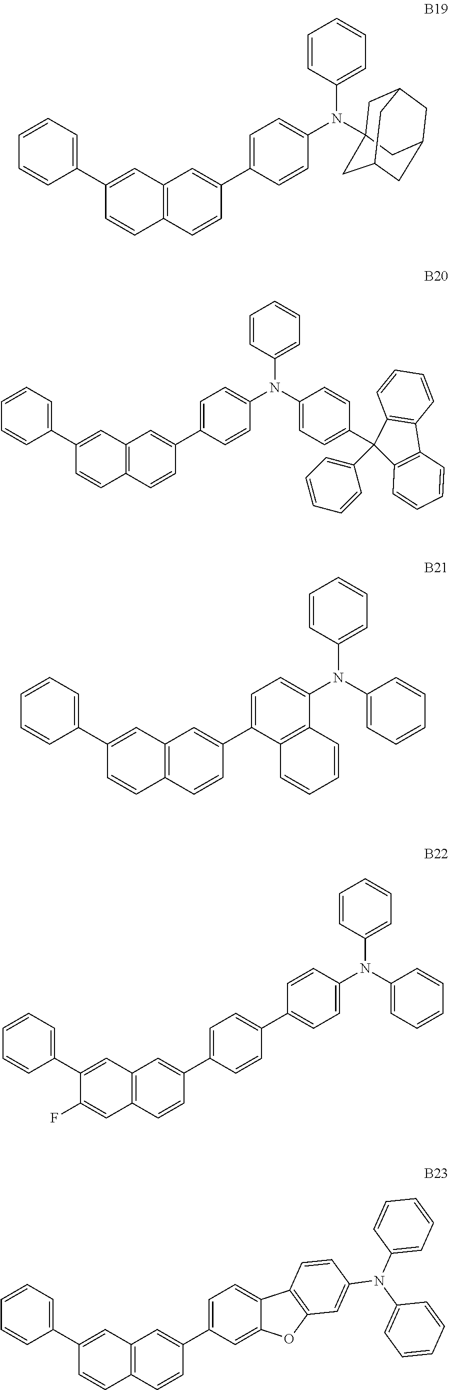 [SMILES: c1ccc(-c2ccc3ccc(-c4ccc(N(c5ccccc5)C56CC7CC(CC(C7)C5)C6)cc4)cc3c2)cc1 ; c1ccc(-c2ccc3ccc(-c4ccc(N(c5ccccc5)c5ccc(C6(c7ccccc7)c7ccccc7-c7ccccc76)cc5)cc4)cc3c2)cc1 ; c1ccc(-c2ccc3ccc(-c4ccc(N(c5ccccc5)c5ccccc5)c5ccccc45)cc3c2)cc1 ; Fc1cc2ccc(-c3ccc(-c4ccc(N(c5ccccc5)c5ccccc5)cc4)cc3)cc2cc1-c1ccccc1 ; c1ccc(-c2ccc3ccc(-c4ccc5c(c4)oc4cc(N(c6ccccc6)c6ccccc6)ccc45)cc3c2)cc1] 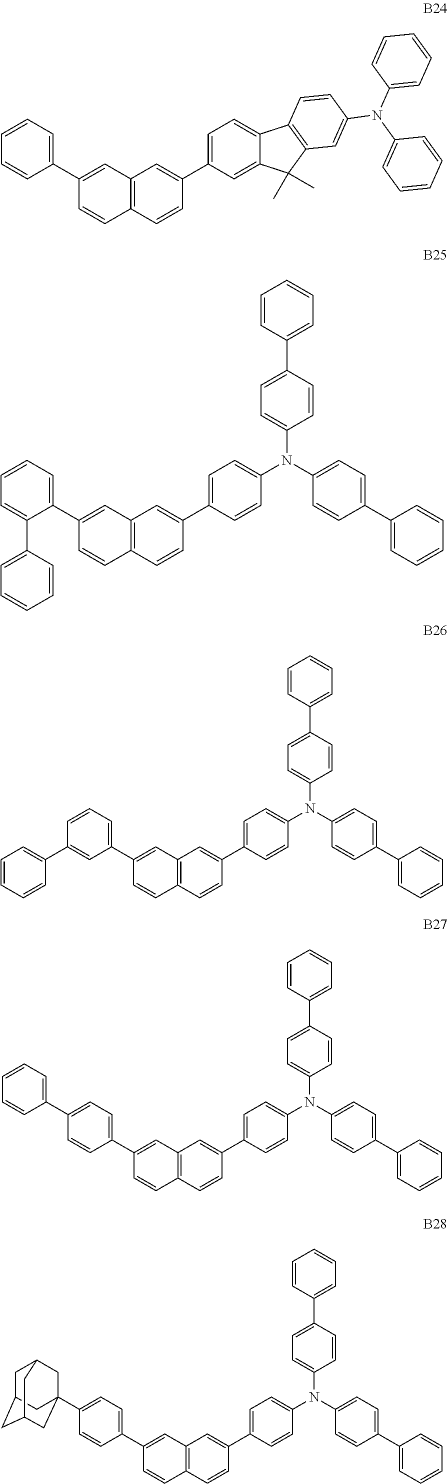 [SMILES: CC1(C)c2cc(-c3ccc4ccc(-c5ccccc5)cc4c3)ccc2-c2ccc(N(c3ccccc3)c3ccccc3)cc21 ; c1ccc(-c2ccc(N(c3ccc(-c4ccccc4)cc3)c3ccc(-c4ccc5ccc(-c6ccccc6-c6ccccc6)cc5c4)cc3)cc2)cc1 ; c1ccc(-c2ccc(N(c3ccc(-c4ccccc4)cc3)c3ccc(-c4ccc5ccc(-c6cccc(-c7ccccc7)c6)cc5c4)cc3)cc2)cc1 ; c1ccc(-c2ccc(-c3ccc4ccc(-c5ccc(N(c6ccc(-c7ccccc7)cc6)c6ccc(-c7ccccc7)cc6)cc5)cc4c3)cc2)cc1 ; c1ccc(-c2ccc(N(c3ccc(-c4ccccc4)cc3)c3ccc(-c4ccc5ccc(-c6ccc(C78CC9CC(CC(C9)C7)C8)cc6)cc5c4)cc3)cc2)cc1] 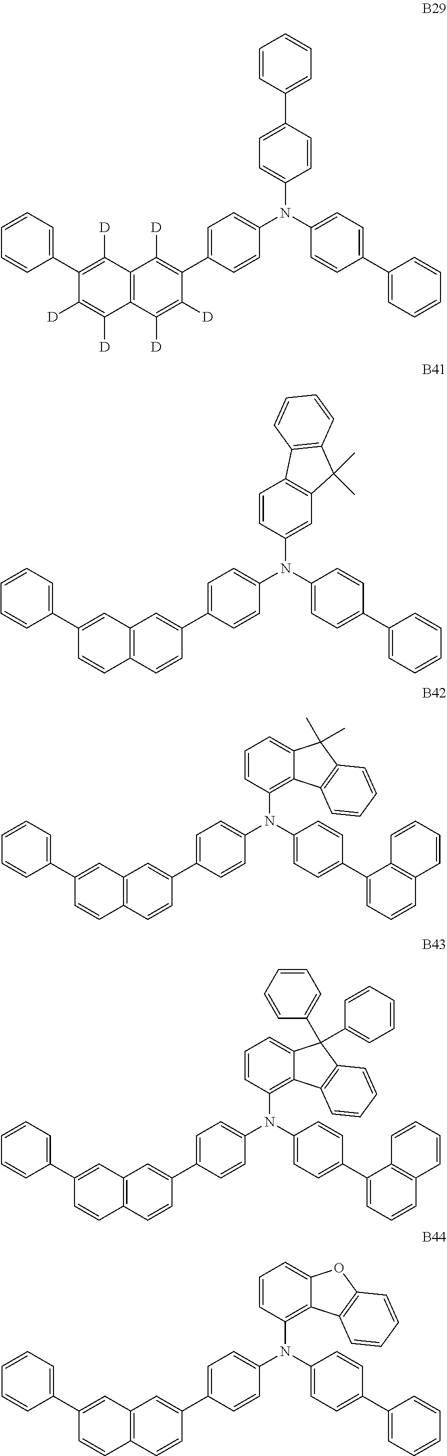 [SMILES: [2H]c1c(-c2ccccc2)c([2H])c2c([2H])c(-c3ccc(N(c4ccc(-c5ccccc5)cc4)c4ccc(-c5ccccc5)cc4)cc3)c([2H])c([2H])c2c1[2H] ; CC1(C)c2ccccc2-c2ccc(N(c3ccc(-c4ccccc4)cc3)c3ccc(-c4ccc5ccc(-c6ccccc6)cc5c4)cc3)cc21 ; CC1(C)c2ccccc2-c2c(N(c3ccc(-c4ccc5ccc(-c6ccccc6)cc5c4)cc3)c3ccc(-c4cccc5ccccc45)cc3)cccc21 ; c1ccc(-c2ccc3ccc(-c4ccc(N(c5ccc(-c6cccc7ccccc67)cc5)c5cccc6c5-c5ccccc5C6(c5ccccc5)c5ccccc5)cc4)cc3c2)cc1 ; c1ccc(-c2ccc(N(c3ccc(-c4ccc5ccc(-c6ccccc6)cc5c4)cc3)c3cccc4oc5ccccc5c34)cc2)cc1] 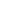 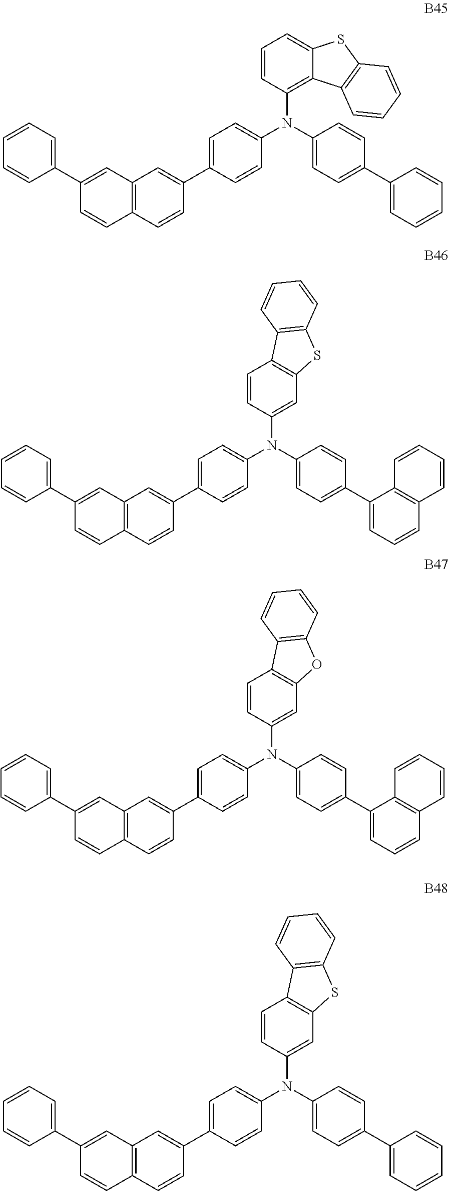 [SMILES: c1ccc(-c2ccc(N(c3ccc(-c4ccc5ccc(-c6ccccc6)cc5c4)cc3)c3cccc4sc5ccccc5c34)cc2)cc1 ; c1ccc(-c2ccc3ccc(-c4ccc(N(c5ccc(-c6cccc7ccccc67)cc5)c5ccc6c(c5)sc5ccccc56)cc4)cc3c2)cc1 ; c1ccc(-c2ccc3ccc(-c4ccc(N(c5ccc(-c6cccc7ccccc67)cc5)c5ccc6c(c5)oc5ccccc56)cc4)cc3c2)cc1 ; c1ccc(-c2ccc(N(c3ccc(-c4ccc5ccc(-c6ccccc6)cc5c4)cc3)c3ccc4c(c3)sc3ccccc34)cc2)cc1] 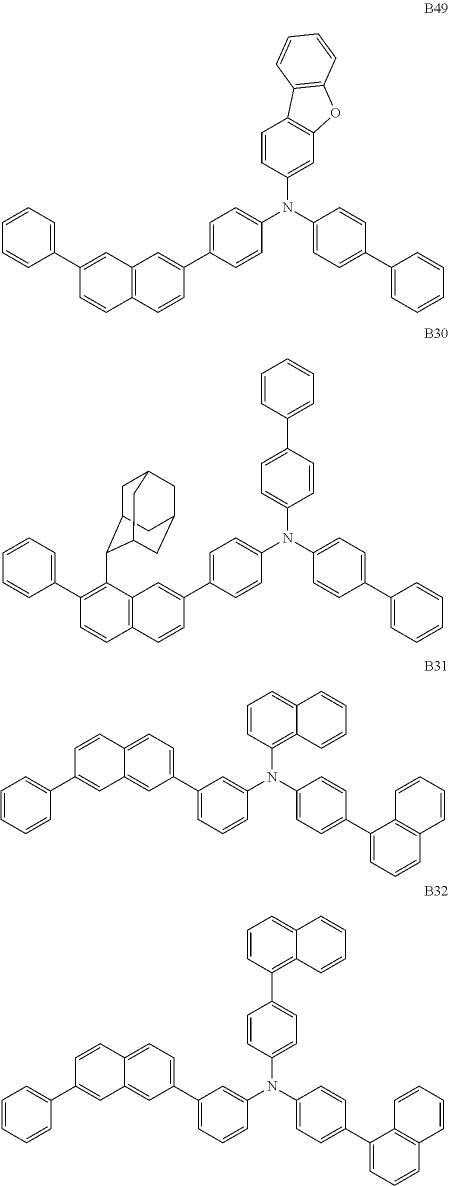 [SMILES: c1ccc(-c2ccc(N(c3ccc(-c4ccc5ccc(-c6ccccc6)cc5c4)cc3)c3ccc4c(c3)oc3ccccc34)cc2)cc1 ; c1ccc(-c2ccc(N(c3ccc(-c4ccccc4)cc3)c3ccc(-c4ccc5ccc(-c6ccccc6)c(C6C7CC8CC(C7)CC6C8)c5c4)cc3)cc2)cc1 ; c1ccc(-c2ccc3ccc(-c4cccc(N(c5ccc(-c6cccc7ccccc67)cc5)c5cccc6ccccc56)c4)cc3c2)cc1 ; c1ccc(-c2ccc3ccc(-c4cccc(N(c5ccc(-c6cccc7ccccc67)cc5)c5ccc(-c6cccc7ccccc67)cc5)c4)cc3c2)cc1] 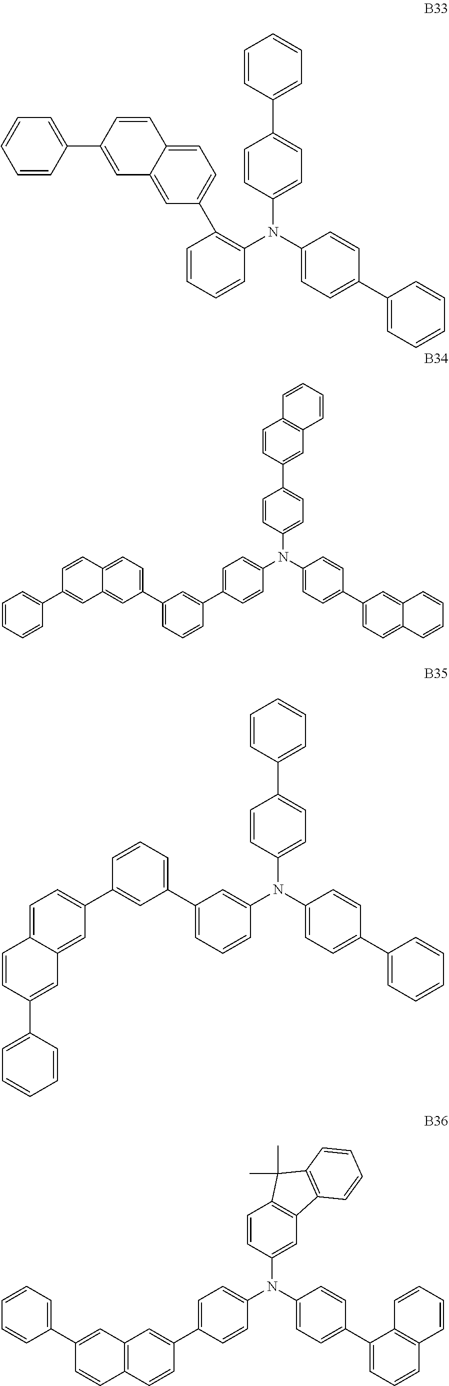 [SMILES: c1ccc(-c2ccc(N(c3ccc(-c4ccccc4)cc3)c3ccccc3-c3ccc4ccc(-c5ccccc5)cc4c3)cc2)cc1 ; c1ccc(-c2ccc3ccc(-c4cccc(-c5ccc(N(c6ccc(-c7ccc8ccccc8c7)cc6)c6ccc(-c7ccc8ccccc8c7)cc6)cc5)c4)cc3c2)cc1 ; c1ccc(-c2ccc(N(c3ccc(-c4ccccc4)cc3)c3cccc(-c4cccc(-c5ccc6ccc(-c7ccccc7)cc6c5)c4)c3)cc2)cc1 ; CC1(C)c2ccccc2-c2cc(N(c3ccc(-c4ccc5ccc(-c6ccccc6)cc5c4)cc3)c3ccc(-c4cccc5ccccc45)cc3)ccc21] 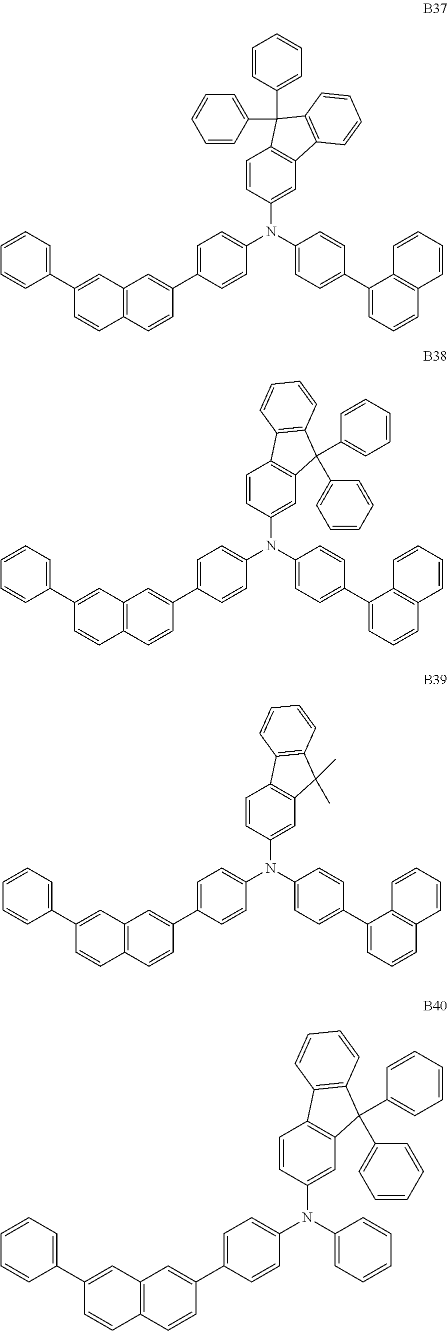 [SMILES: c1ccc(-c2ccc3ccc(-c4ccc(N(c5ccc(-c6cccc7ccccc67)cc5)c5ccc6c(c5)-c5ccccc5C6(c5ccccc5)c5ccccc5)cc4)cc3c2)cc1 ; c1ccc(-c2ccc3ccc(-c4ccc(N(c5ccc(-c6cccc7ccccc67)cc5)c5ccc6c(c5)C(c5ccccc5)(c5ccccc5)c5ccccc5-6)cc4)cc3c2)cc1 ; CC1(C)c2ccccc2-c2ccc(N(c3ccc(-c4ccc5ccc(-c6ccccc6)cc5c4)cc3)c3ccc(-c4cccc5ccccc45)cc3)cc21 ; c1ccc(-c2ccc3ccc(-c4ccc(N(c5ccccc5)c5ccc6c(c5)C(c5ccccc5)(c5ccccc5)c5ccccc5-6)cc4)cc3c2)cc1] 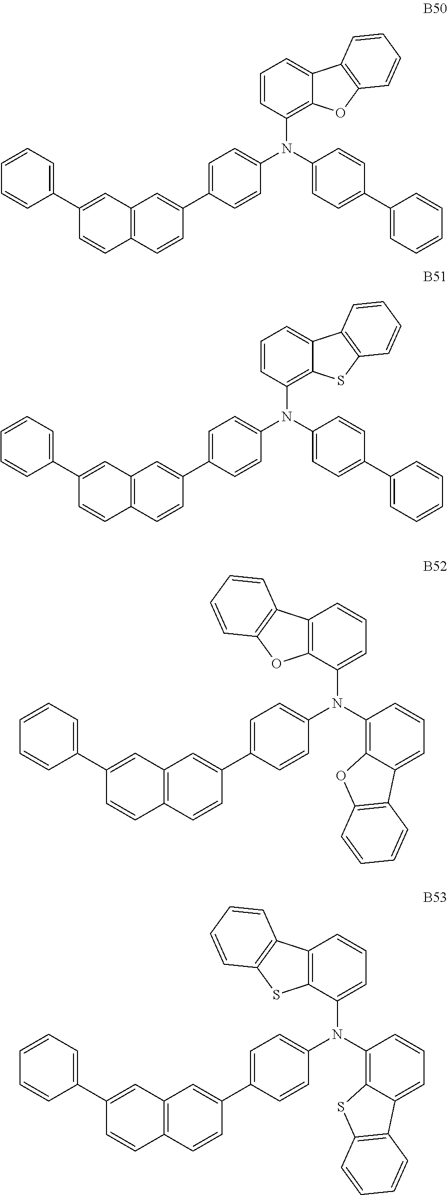 [SMILES: c1ccc(-c2ccc(N(c3ccc(-c4ccc5ccc(-c6ccccc6)cc5c4)cc3)c3cccc4c3oc3ccccc34)cc2)cc1 ; c1ccc(-c2ccc(N(c3ccc(-c4ccc5ccc(-c6ccccc6)cc5c4)cc3)c3cccc4c3sc3ccccc34)cc2)cc1 ; c1ccc(-c2ccc3ccc(-c4ccc(N(c5cccc6c5oc5ccccc56)c5cccc6c5oc5ccccc56)cc4)cc3c2)cc1 ; c1ccc(-c2ccc3ccc(-c4ccc(N(c5cccc6c5sc5ccccc56)c5cccc6c5sc5ccccc56)cc4)cc3c2)cc1] 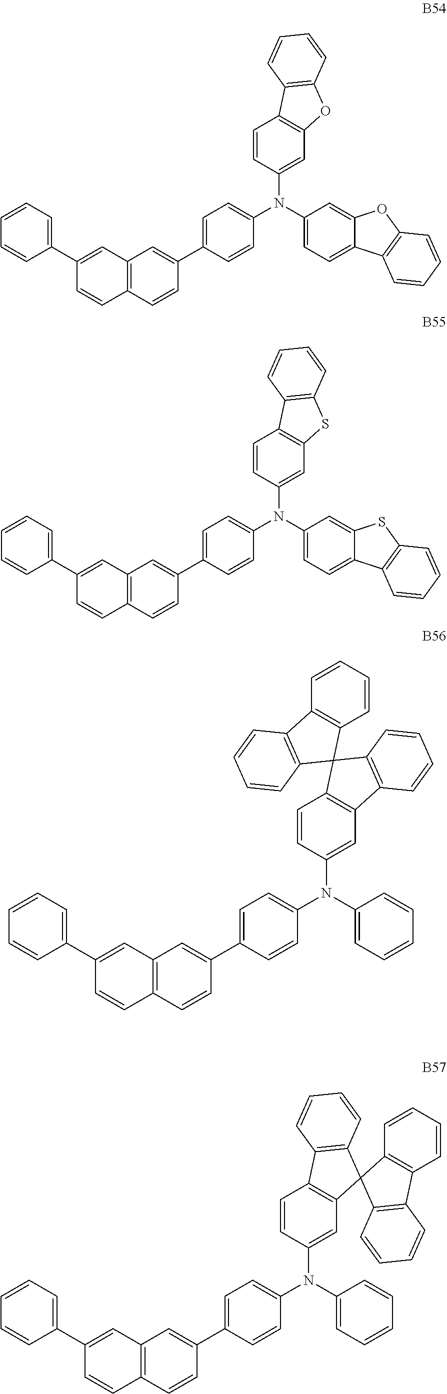 [SMILES: c1ccc(-c2ccc3ccc(-c4ccc(N(c5ccc6c(c5)oc5ccccc56)c5ccc6c(c5)oc5ccccc56)cc4)cc3c2)cc1 ; c1ccc(-c2ccc3ccc(-c4ccc(N(c5ccc6c(c5)sc5ccccc56)c5ccc6c(c5)sc5ccccc56)cc4)cc3c2)cc1 ; c1ccc(-c2ccc3ccc(-c4ccc(N(c5ccccc5)c5ccc6c(c5)-c5ccccc5C65c6ccccc6-c6ccccc65)cc4)cc3c2)cc1 ; c1ccc(-c2ccc3ccc(-c4ccc(N(c5ccccc5)c5ccc6c(c5)C5(c7ccccc7-c7ccccc75)c5ccccc5-6)cc4)cc3c2)cc1] 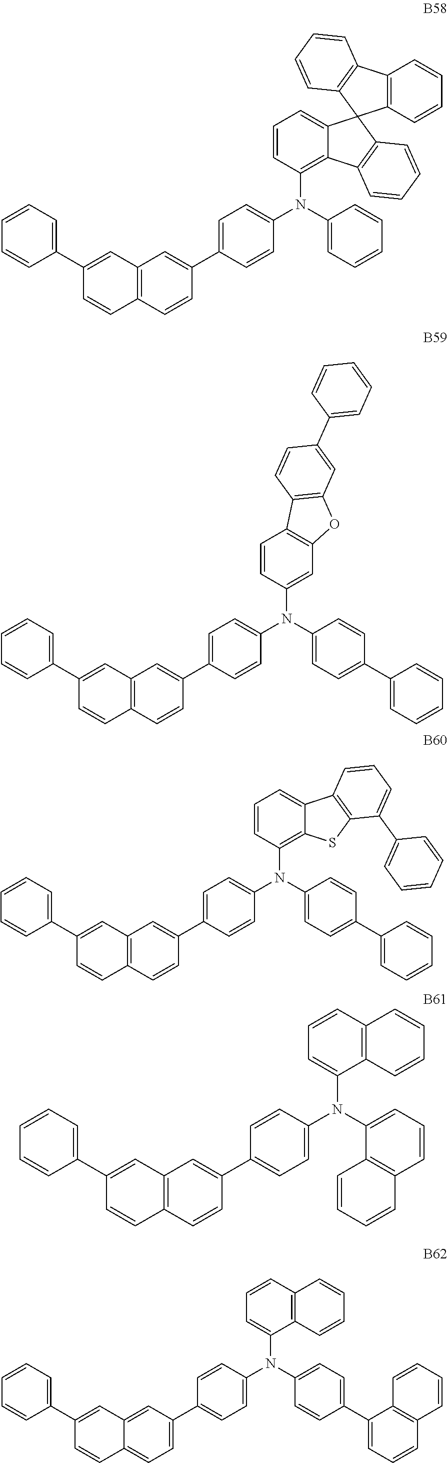 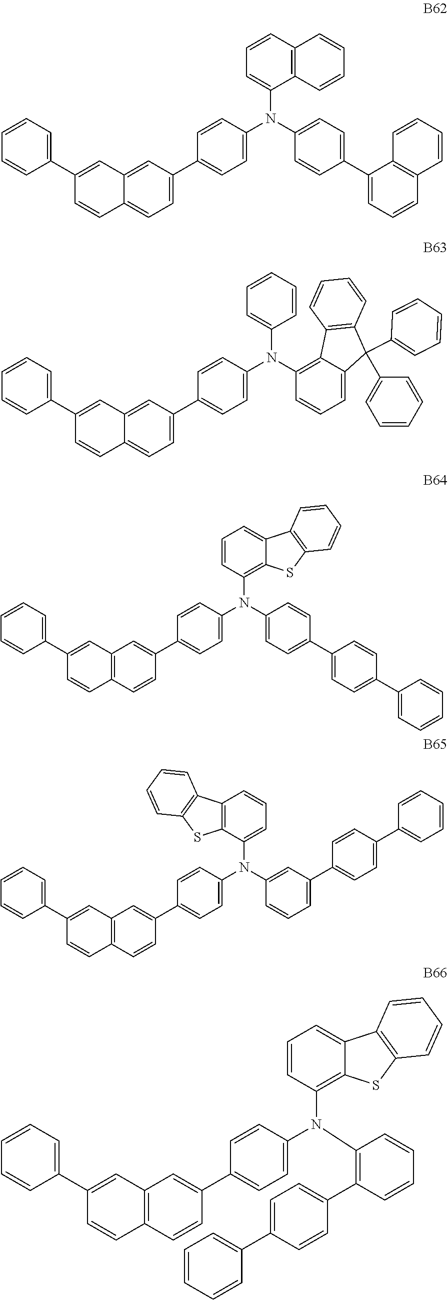 [SMILES: c1ccc(-c2ccc3ccc(-c4ccc(N(c5ccc(-c6cccc7ccccc67)cc5)c5cccc6ccccc56)cc4)cc3c2)cc1 ; c1ccc(-c2ccc3ccc(-c4ccc(N(c5ccccc5)c5cccc6c5-c5ccccc5C6(c5ccccc5)c5ccccc5)cc4)cc3c2)cc1 ; c1ccc(-c2ccc(-c3ccc(N(c4ccc(-c5ccc6ccc(-c7ccccc7)cc6c5)cc4)c4cccc5c4sc4ccccc45)cc3)cc2)cc1 ; c1ccc(-c2ccc(-c3cccc(N(c4ccc(-c5ccc6ccc(-c7ccccc7)cc6c5)cc4)c4cccc5c4sc4ccccc45)c3)cc2)cc1 ; c1ccc(-c2ccc(-c3ccccc3N(c3ccc(-c4ccc5ccc(-c6ccccc6)cc5c4)cc3)c3cccc4c3sc3ccccc34)cc2)cc1] 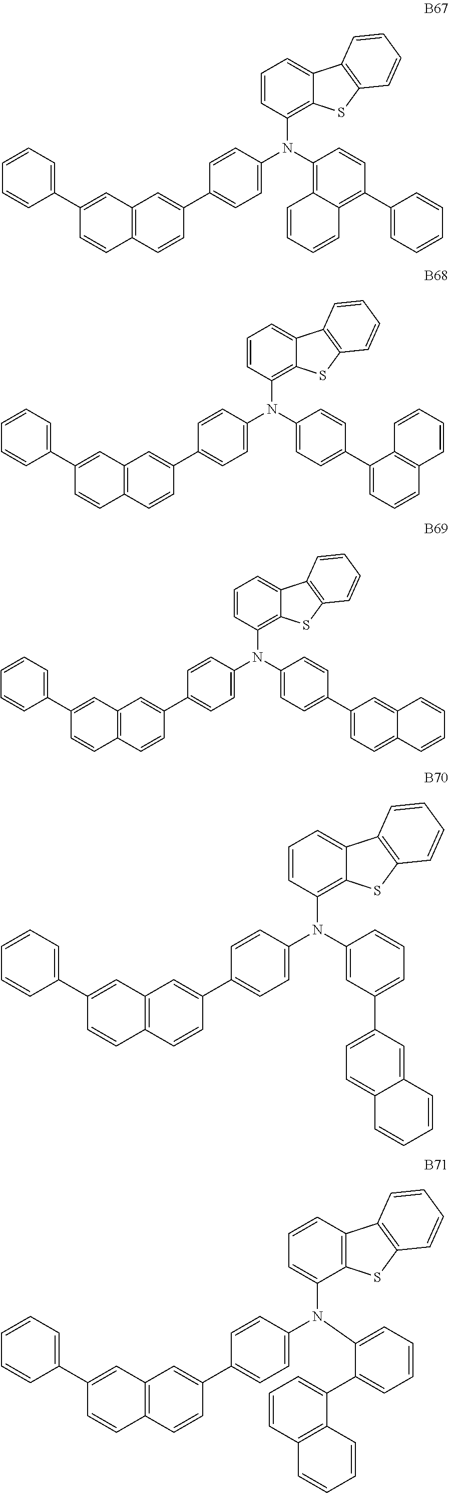 [SMILES: c1ccc(-c2ccc3ccc(-c4ccc(N(c5ccc(-c6ccccc6)c6ccccc56)c5cccc6c5sc5ccccc56)cc4)cc3c2)cc1 ; c1ccc(-c2ccc3ccc(-c4ccc(N(c5ccc(-c6cccc7ccccc67)cc5)c5cccc6c5sc5ccccc56)cc4)cc3c2)cc1 ; c1ccc(-c2ccc3ccc(-c4ccc(N(c5ccc(-c6ccc7ccccc7c6)cc5)c5cccc6c5sc5ccccc56)cc4)cc3c2)cc1 ; c1ccc(-c2ccc3ccc(-c4ccc(N(c5cccc(-c6ccc7ccccc7c6)c5)c5cccc6c5sc5ccccc56)cc4)cc3c2)cc1 ; c1ccc(-c2ccc3ccc(-c4ccc(N(c5ccccc5-c5cccc6ccccc56)c5cccc6c5sc5ccccc56)cc4)cc3c2)cc1] 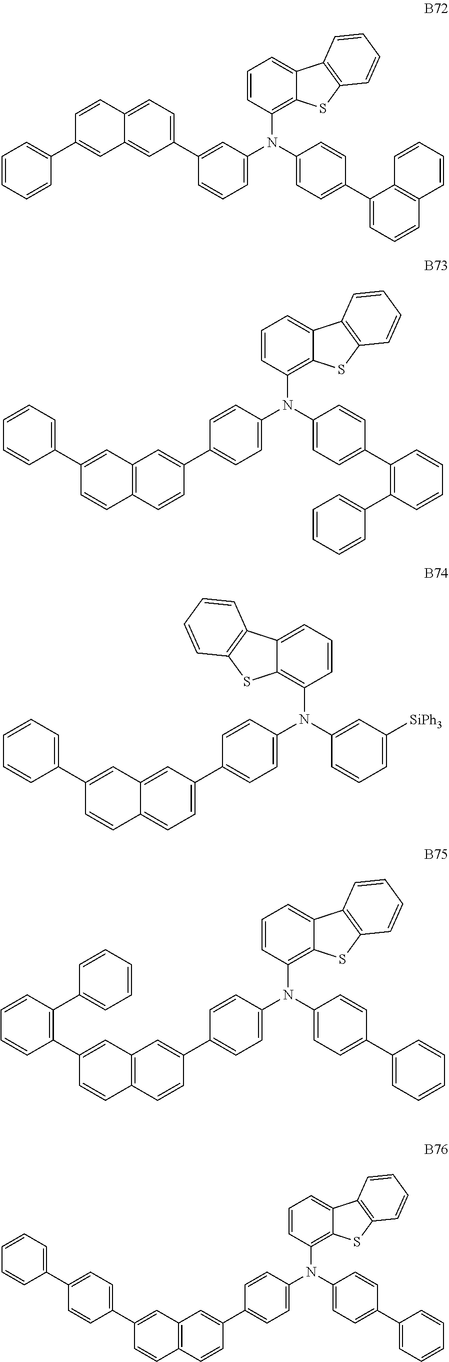 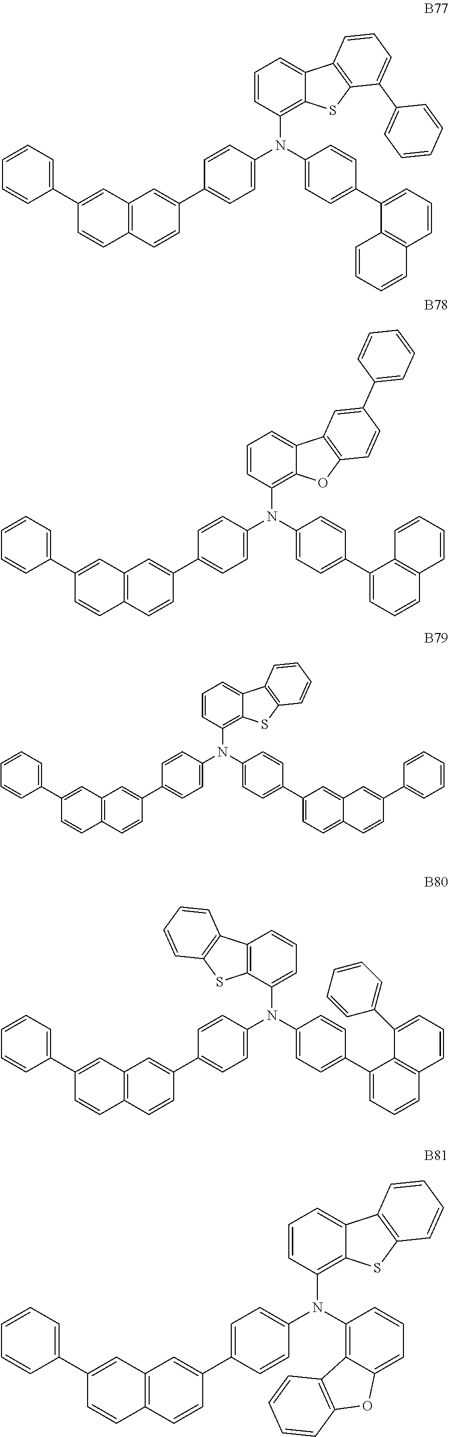 [SMILES: c1ccc(-c2ccc3ccc(-c4ccc(N(c5ccc(-c6cccc7ccccc67)cc5)c5cccc6c5sc5c(-c7ccccc7)cccc56)cc4)cc3c2)cc1 ; c1ccc(-c2ccc3ccc(-c4ccc(N(c5ccc(-c6cccc7ccccc67)cc5)c5cccc6c5oc5ccc(-c7ccccc7)cc56)cc4)cc3c2)cc1 ; c1ccc(-c2ccc3ccc(-c4ccc(N(c5ccc(-c6ccc7ccc(-c8ccccc8)cc7c6)cc5)c5cccc6c5sc5ccccc56)cc4)cc3c2)cc1 ; c1ccc(-c2ccc3ccc(-c4ccc(N(c5ccc(-c6cccc7cccc(-c8ccccc8)c67)cc5)c5cccc6c5sc5ccccc56)cc4)cc3c2)cc1 ; c1ccc(-c2ccc3ccc(-c4ccc(N(c5cccc6c5sc5ccccc56)c5cccc6oc7ccccc7c56)cc4)cc3c2)cc1] 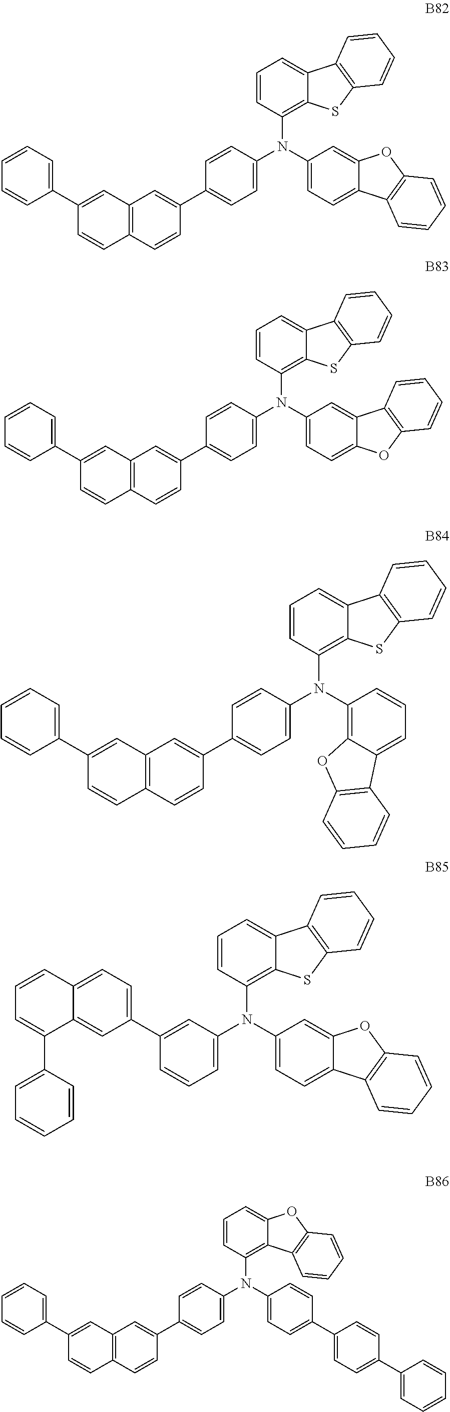 [SMILES: c1ccc(-c2ccc3ccc(-c4ccc(N(c5ccc6c(c5)oc5ccccc56)c5cccc6c5sc5ccccc56)cc4)cc3c2)cc1 ; c1ccc(-c2ccc3ccc(-c4ccc(N(c5ccc6oc7ccccc7c6c5)c5cccc6c5sc5ccccc56)cc4)cc3c2)cc1 ; c1ccc(-c2ccc3ccc(-c4ccc(N(c5cccc6c5oc5ccccc56)c5cccc6c5sc5ccccc56)cc4)cc3c2)cc1 ; c1ccc(-c2cccc3ccc(-c4cccc(N(c5ccc6c(c5)oc5ccccc56)c5cccc6c5sc5ccccc56)c4)cc23)cc1 ; c1ccc(-c2ccc(-c3ccc(N(c4ccc(-c5ccc6ccc(-c7ccccc7)cc6c5)cc4)c4cccc5oc6ccccc6c45)cc3)cc2)cc1] 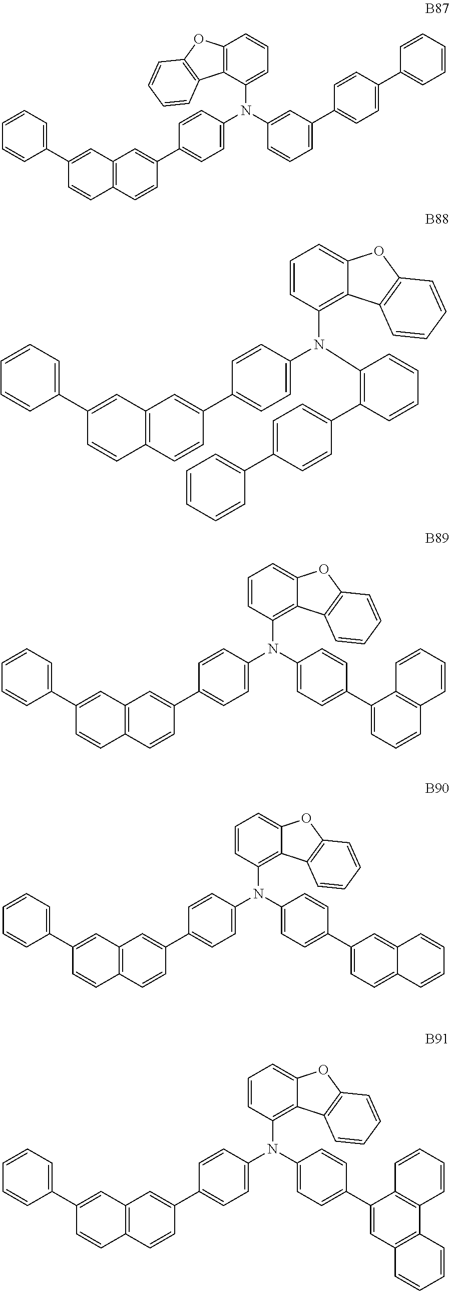 [SMILES: c1ccc(-c2ccc(-c3cccc(N(c4ccc(-c5ccc6ccc(-c7ccccc7)cc6c5)cc4)c4cccc5oc6ccccc6c45)c3)cc2)cc1 ; c1ccc(-c2ccc(-c3ccccc3N(c3ccc(-c4ccc5ccc(-c6ccccc6)cc5c4)cc3)c3cccc4oc5ccccc5c34)cc2)cc1 ; c1ccc(-c2ccc3ccc(-c4ccc(N(c5ccc(-c6cccc7ccccc67)cc5)c5cccc6oc7ccccc7c56)cc4)cc3c2)cc1 ; c1ccc(-c2ccc3ccc(-c4ccc(N(c5ccc(-c6ccc7ccccc7c6)cc5)c5cccc6oc7ccccc7c56)cc4)cc3c2)cc1 ; c1ccc(-c2ccc3ccc(-c4ccc(N(c5ccc(-c6cc7ccccc7c7ccccc67)cc5)c5cccc6oc7ccccc7c56)cc4)cc3c2)cc1] 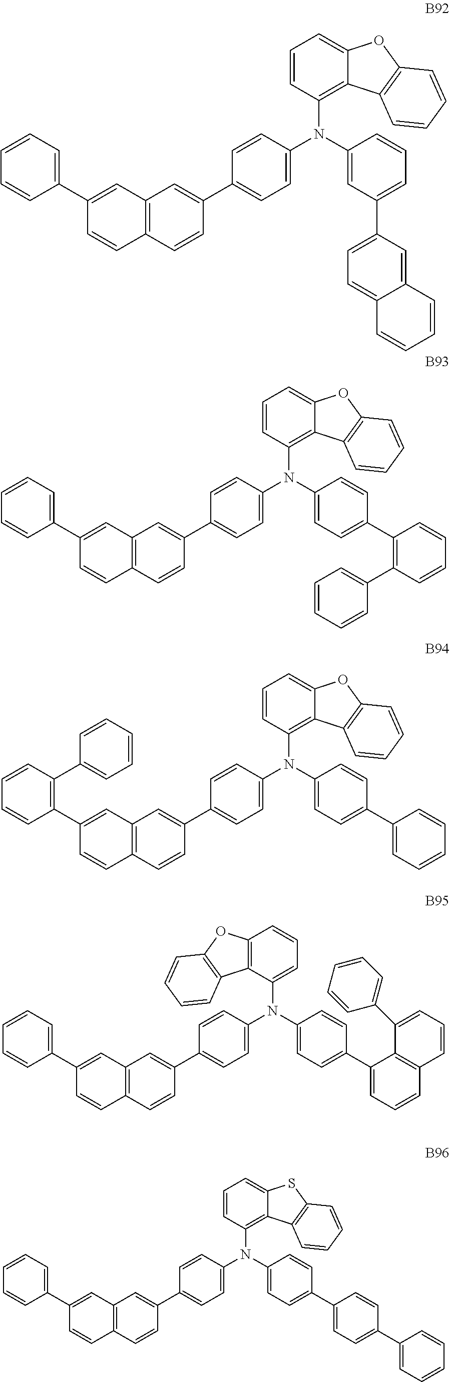 [SMILES: c1ccc(-c2ccc3ccc(-c4ccc(N(c5cccc(-c6ccc7ccccc7c6)c5)c5cccc6oc7ccccc7c56)cc4)cc3c2)cc1 ; c1ccc(-c2ccc3ccc(-c4ccc(N(c5ccc(-c6ccccc6-c6ccccc6)cc5)c5cccc6oc7ccccc7c56)cc4)cc3c2)cc1 ; c1ccc(-c2ccc(N(c3ccc(-c4ccc5ccc(-c6ccccc6-c6ccccc6)cc5c4)cc3)c3cccc4oc5ccccc5c34)cc2)cc1 ; c1ccc(-c2ccc3ccc(-c4ccc(N(c5ccc(-c6cccc7cccc(-c8ccccc8)c67)cc5)c5cccc6oc7ccccc7c56)cc4)cc3c2)cc1 ; c1ccc(-c2ccc(-c3ccc(N(c4ccc(-c5ccc6ccc(-c7ccccc7)cc6c5)cc4)c4cccc5sc6ccccc6c45)cc3)cc2)cc1] 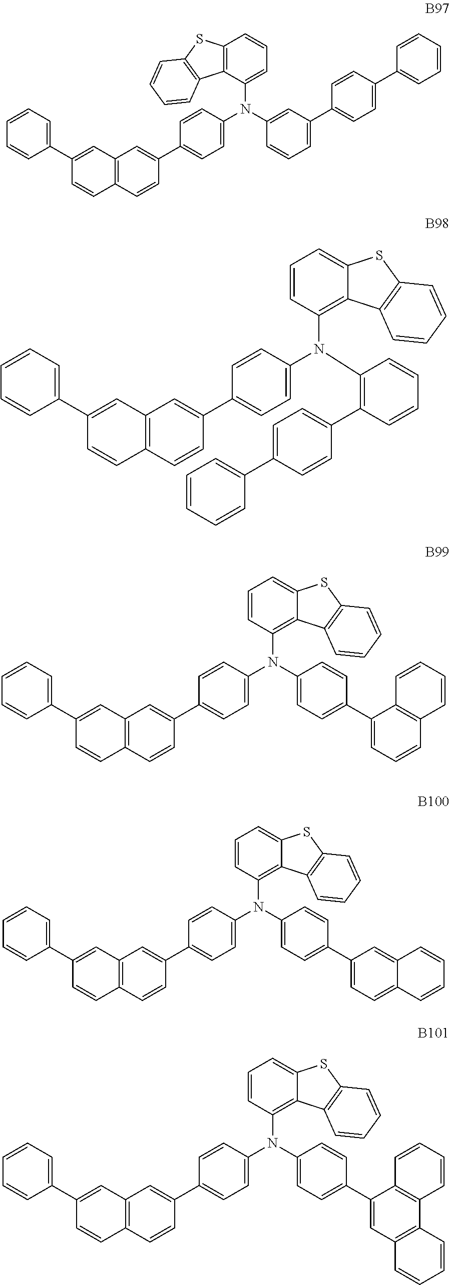 [SMILES: c1ccc(-c2ccc(-c3cccc(N(c4ccc(-c5ccc6ccc(-c7ccccc7)cc6c5)cc4)c4cccc5sc6ccccc6c45)c3)cc2)cc1 ; c1ccc(-c2ccc(-c3ccccc3N(c3ccc(-c4ccc5ccc(-c6ccccc6)cc5c4)cc3)c3cccc4sc5ccccc5c34)cc2)cc1 ; c1ccc(-c2ccc3ccc(-c4ccc(N(c5ccc(-c6cccc7ccccc67)cc5)c5cccc6sc7ccccc7c56)cc4)cc3c2)cc1 ; c1ccc(-c2ccc3ccc(-c4ccc(N(c5ccc(-c6ccc7ccccc7c6)cc5)c5cccc6sc7ccccc7c56)cc4)cc3c2)cc1 ; c1ccc(-c2ccc3ccc(-c4ccc(N(c5ccc(-c6cc7ccccc7c7ccccc67)cc5)c5cccc6sc7ccccc7c56)cc4)cc3c2)cc1] 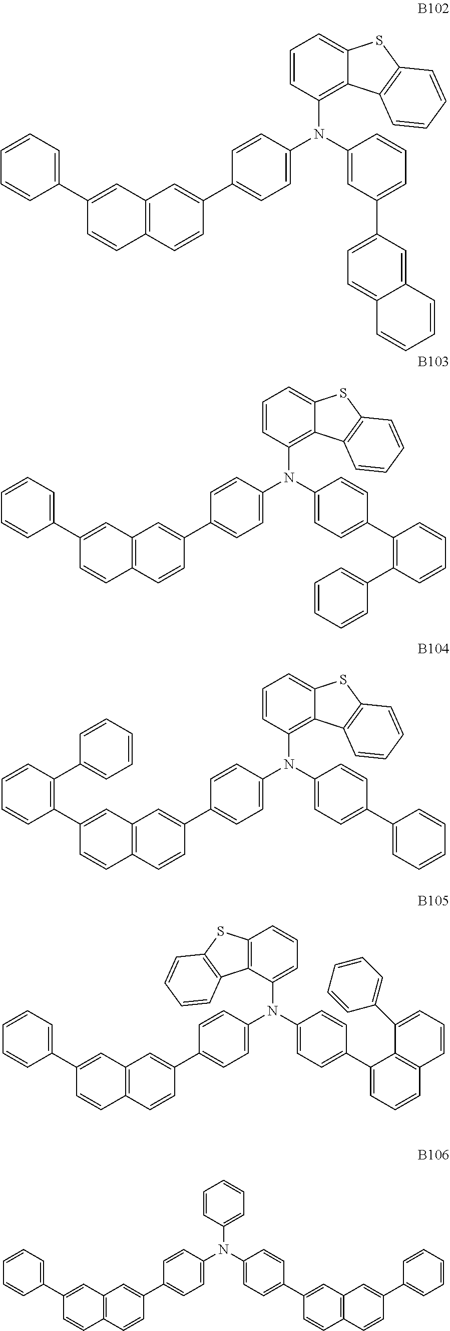 [SMILES: c1ccc(-c2ccc3ccc(-c4ccc(N(c5cccc(-c6ccc7ccccc7c6)c5)c5cccc6sc7ccccc7c56)cc4)cc3c2)cc1 ; c1ccc(-c2ccc3ccc(-c4ccc(N(c5ccc(-c6ccccc6-c6ccccc6)cc5)c5cccc6sc7ccccc7c56)cc4)cc3c2)cc1 ; c1ccc(-c2ccc(N(c3ccc(-c4ccc5ccc(-c6ccccc6-c6ccccc6)cc5c4)cc3)c3cccc4sc5ccccc5c34)cc2)cc1 ; c1ccc(-c2ccc3ccc(-c4ccc(N(c5ccc(-c6cccc7cccc(-c8ccccc8)c67)cc5)c5cccc6sc7ccccc7c56)cc4)cc3c2)cc1 ; c1ccc(-c2ccc3ccc(-c4ccc(N(c5ccccc5)c5ccc(-c6ccc7ccc(-c8ccccc8)cc7c6)cc5)cc4)cc3c2)cc1] 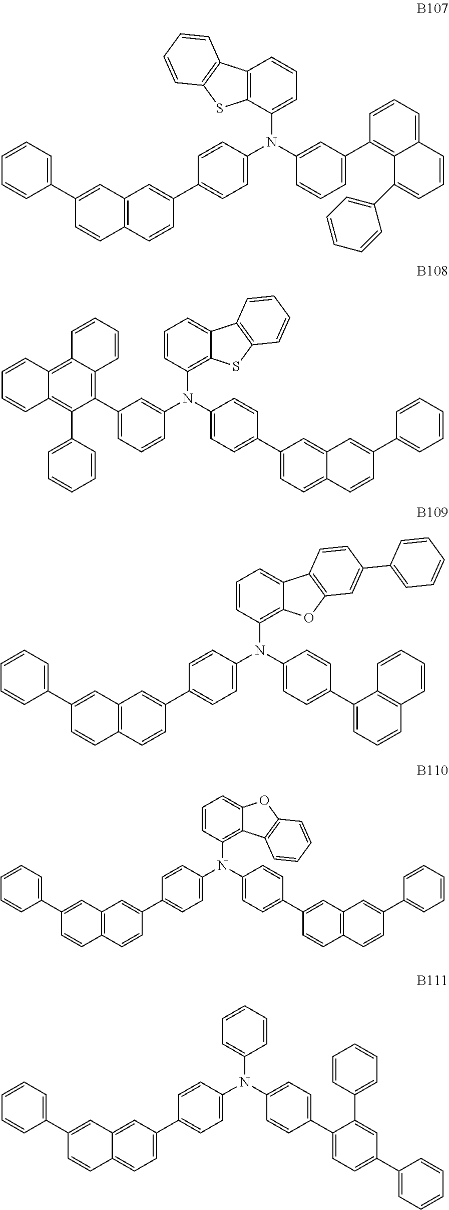 [SMILES: c1ccc(-c2ccc3ccc(-c4ccc(N(c5cccc(-c6cccc7cccc(-c8ccccc8)c67)c5)c5cccc6c5sc5ccccc56)cc4)cc3c2)cc1 ; c1ccc(-c2ccc3ccc(-c4ccc(N(c5cccc(-c6c(-c7ccccc7)c7ccccc7c7ccccc67)c5)c5cccc6c5sc5ccccc56)cc4)cc3c2)cc1 ; c1ccc(-c2ccc3ccc(-c4ccc(N(c5ccc(-c6cccc7ccccc67)cc5)c5cccc6c5oc5cc(-c7ccccc7)ccc56)cc4)cc3c2)cc1 ; c1ccc(-c2ccc3ccc(-c4ccc(N(c5ccc(-c6ccc7ccc(-c8ccccc8)cc7c6)cc5)c5cccc6oc7ccccc7c56)cc4)cc3c2)cc1 ; c1ccc(-c2ccc(-c3ccc(N(c4ccccc4)c4ccc(-c5ccc6ccc(-c7ccccc7)cc6c5)cc4)cc3)c(-c3ccccc3)c2)cc1] 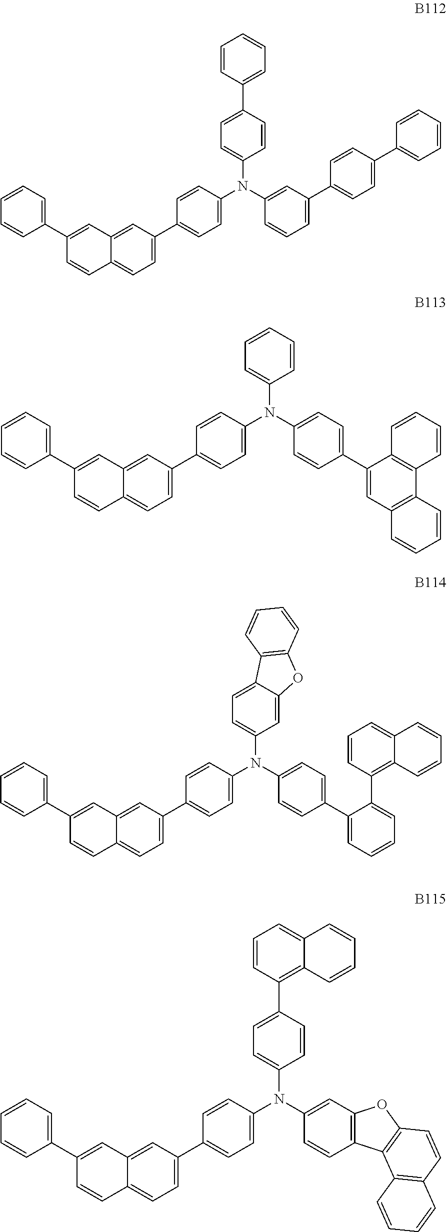 [SMILES: c1ccc(-c2ccc(-c3cccc(N(c4ccc(-c5ccccc5)cc4)c4ccc(-c5ccc6ccc(-c7ccccc7)cc6c5)cc4)c3)cc2)cc1 ; c1ccc(-c2ccc3ccc(-c4ccc(N(c5ccccc5)c5ccc(-c6cc7ccccc7c7ccccc67)cc5)cc4)cc3c2)cc1 ; c1ccc(-c2ccc3ccc(-c4ccc(N(c5ccc(-c6ccccc6-c6cccc7ccccc67)cc5)c5ccc6c(c5)oc5ccccc56)cc4)cc3c2)cc1 ; c1ccc(-c2ccc3ccc(-c4ccc(N(c5ccc(-c6cccc7ccccc67)cc5)c5ccc6c(c5)oc5ccc7ccccc7c56)cc4)cc3c2)cc1] 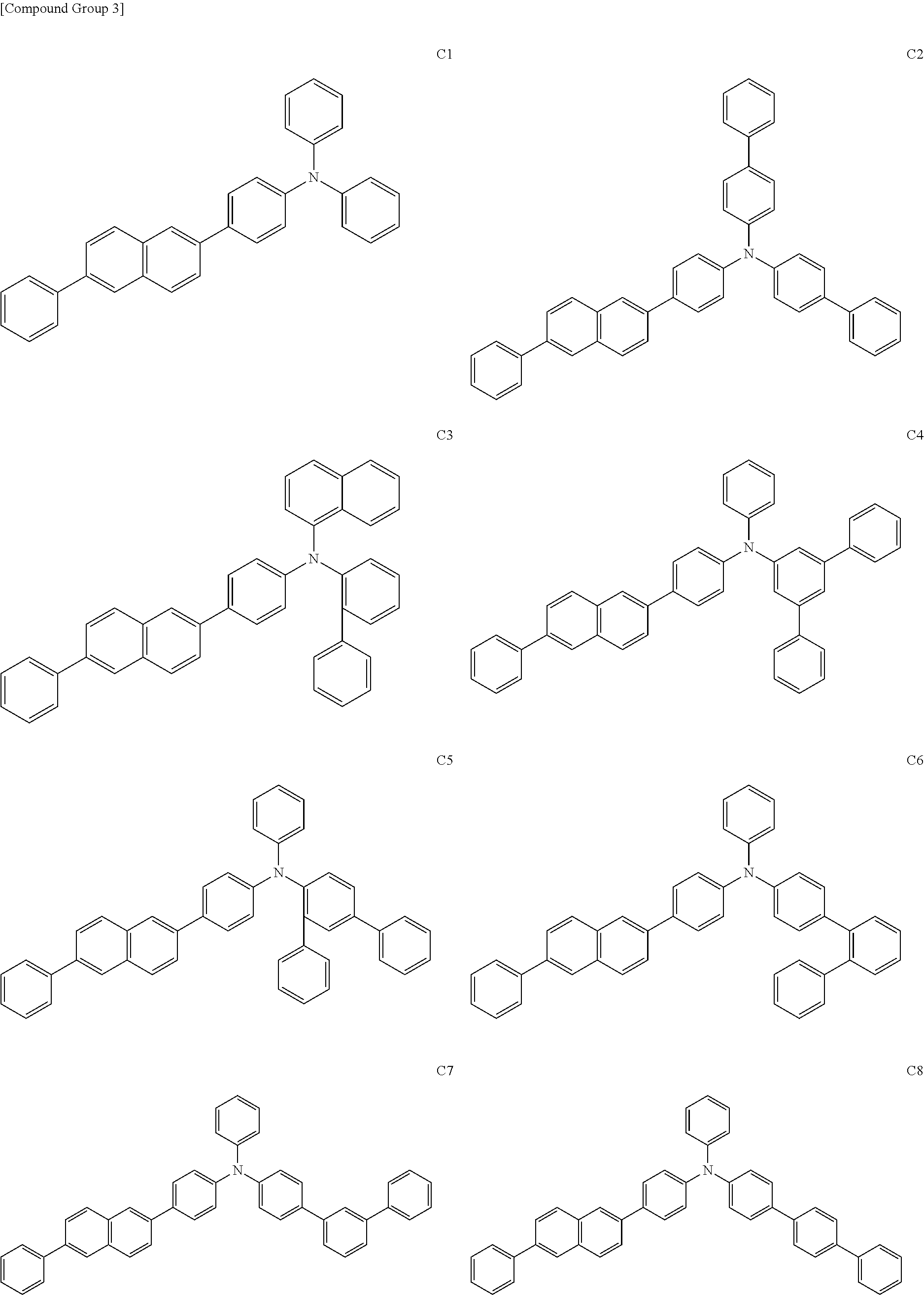 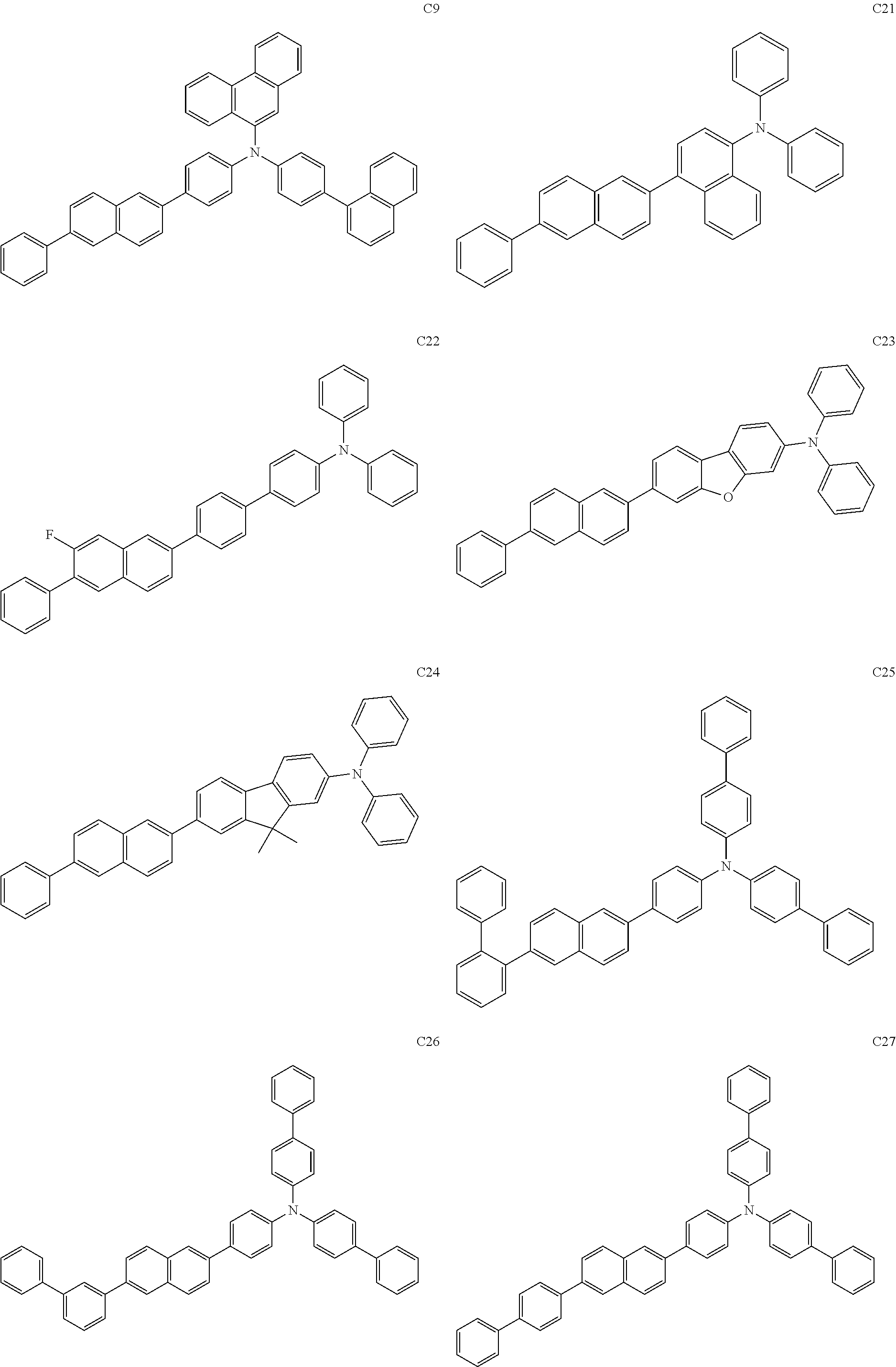 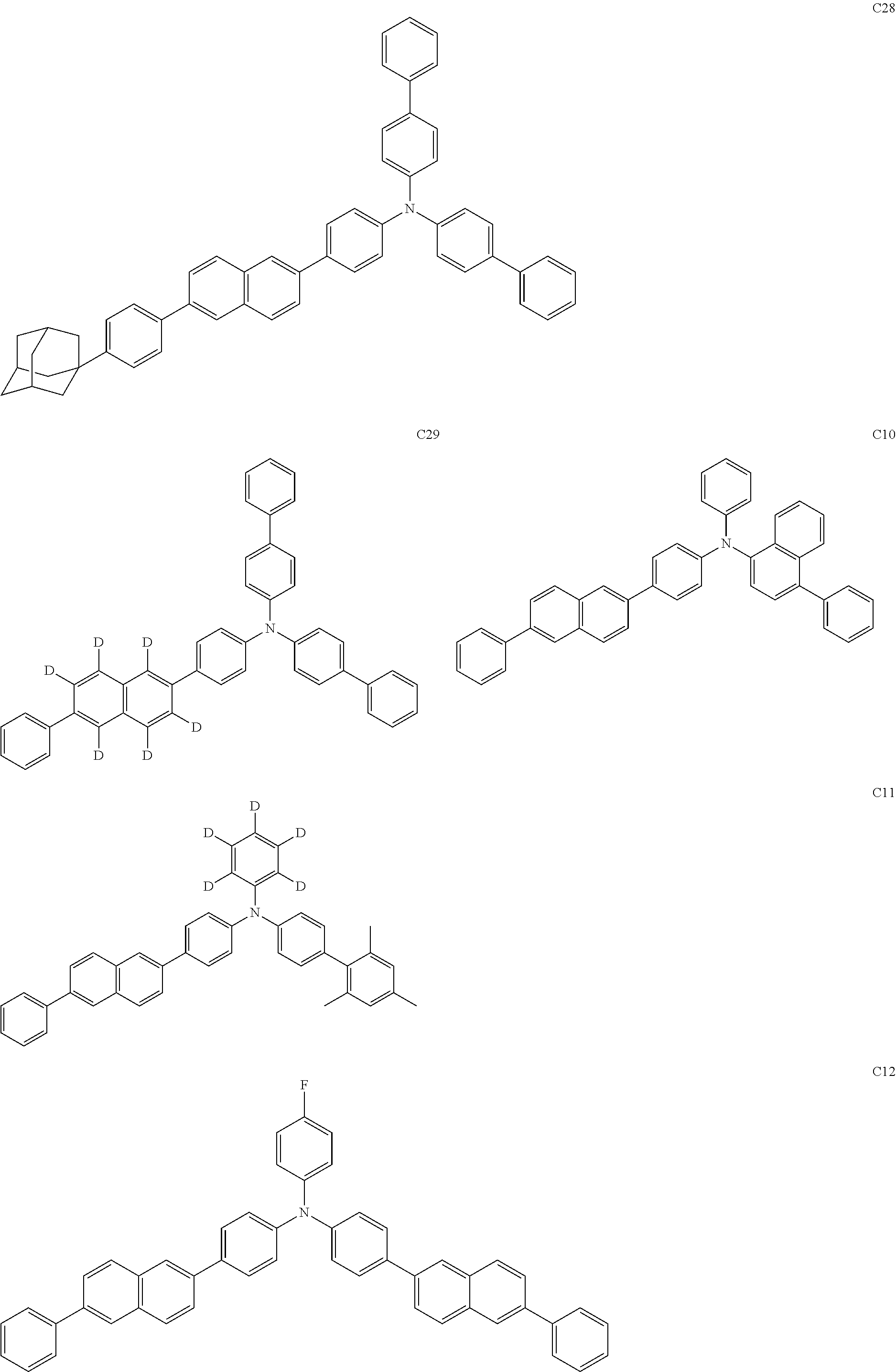 [SMILES: c1ccc(-c2ccc(N(c3ccc(-c4ccccc4)cc3)c3ccc(-c4ccc5cc(-c6ccc(C78CC9CC(CC(C9)C7)C8)cc6)ccc5c4)cc3)cc2)cc1 ; [2H]c1c(-c2ccc(N(c3ccc(-c4ccccc4)cc3)c3ccc(-c4ccccc4)cc3)cc2)c([2H])c2c([2H])c([2H])c(-c3ccccc3)c([2H])c2c1[2H] ; c1ccc(-c2ccc3cc(-c4ccc(N(c5ccccc5)c5ccc(-c6ccccc6)c6ccccc56)cc4)ccc3c2)cc1 ; [2H]c1c([2H])c([2H])c(N(c2ccc(-c3ccc4cc(-c5ccccc5)ccc4c3)cc2)c2ccc(-c3c(C)cc(C)cc3C)cc2)c([2H])c1[2H] ; Fc1ccc(N(c2ccc(-c3ccc4cc(-c5ccccc5)ccc4c3)cc2)c2ccc(-c3ccc4cc(-c5ccccc5)ccc4c3)cc2)cc1] 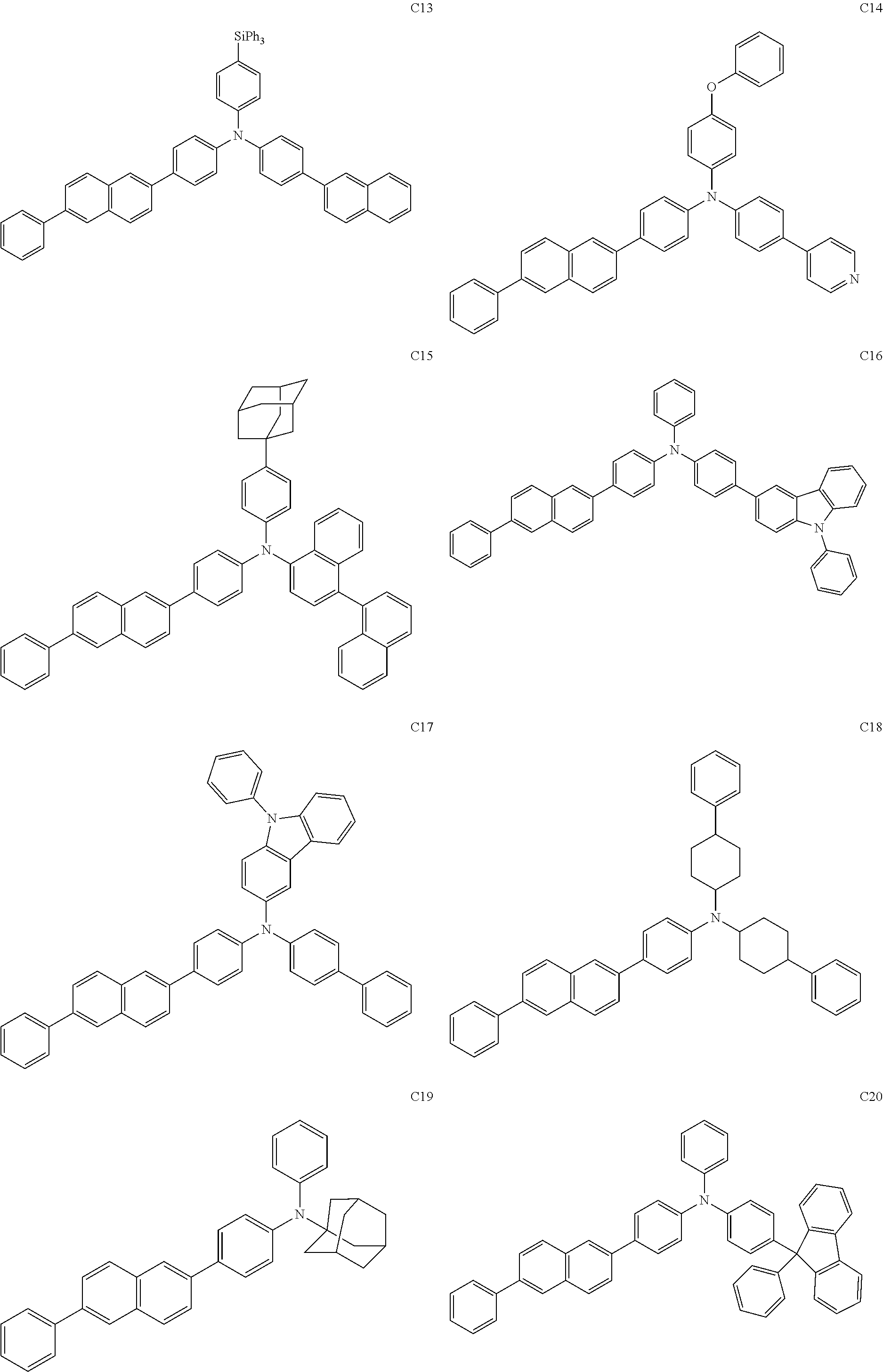 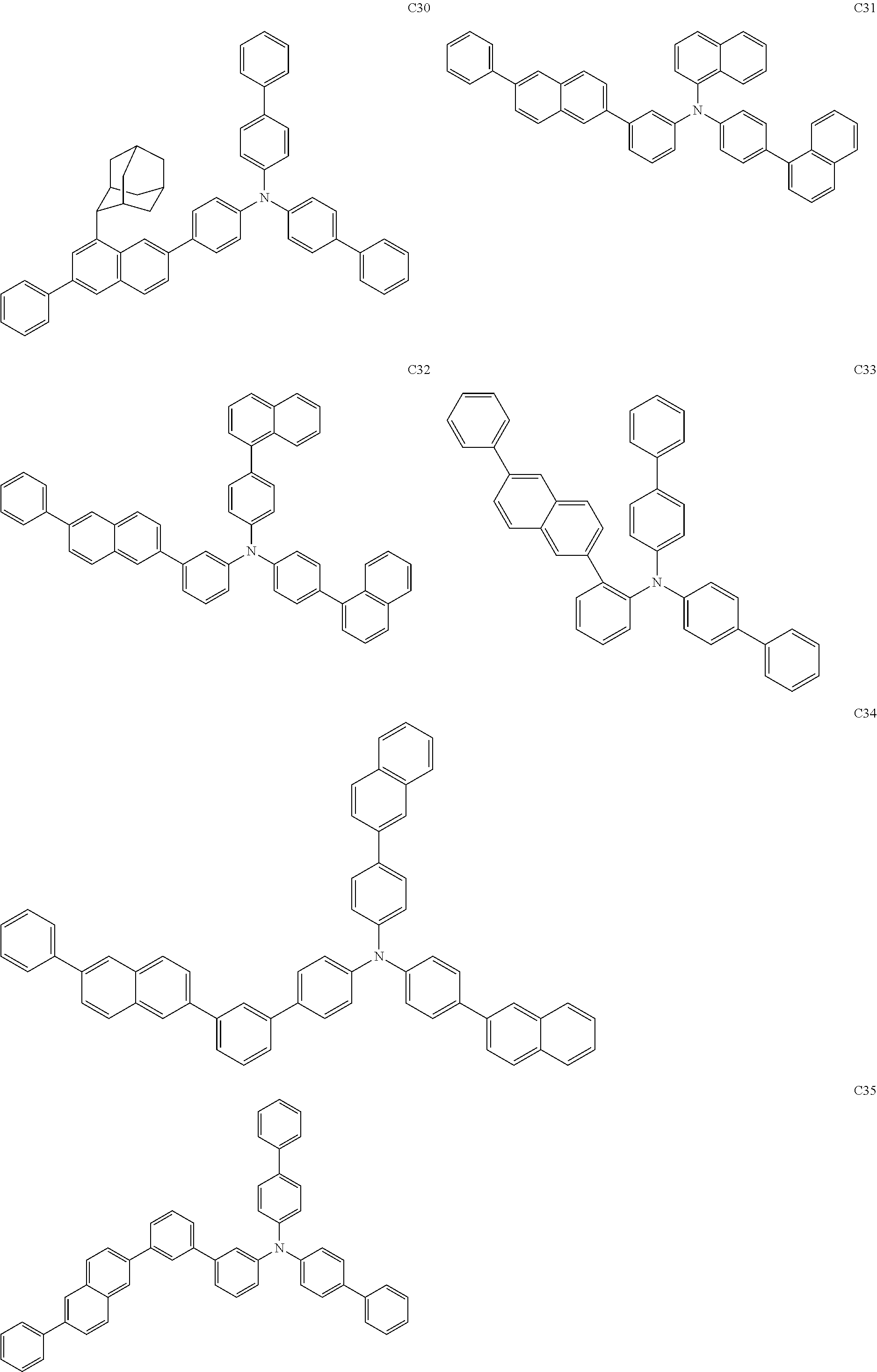 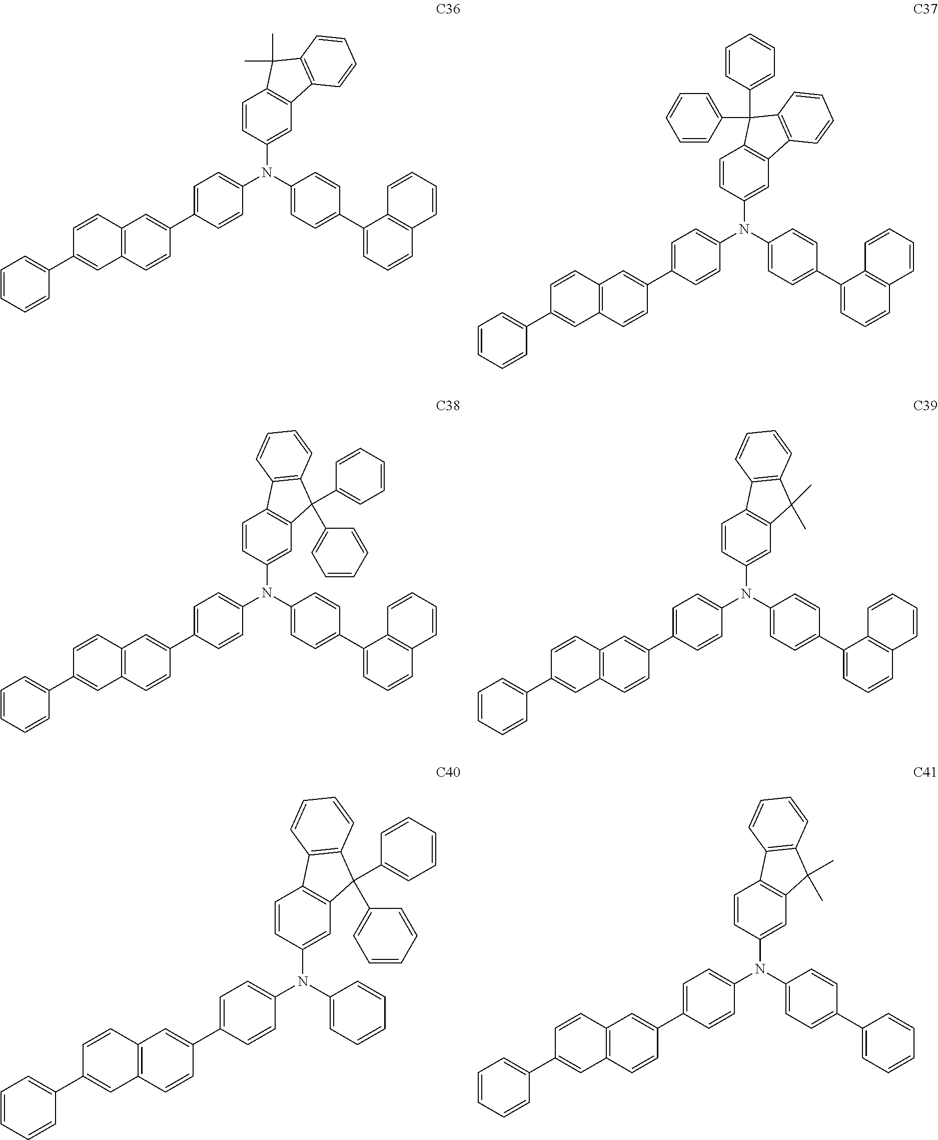 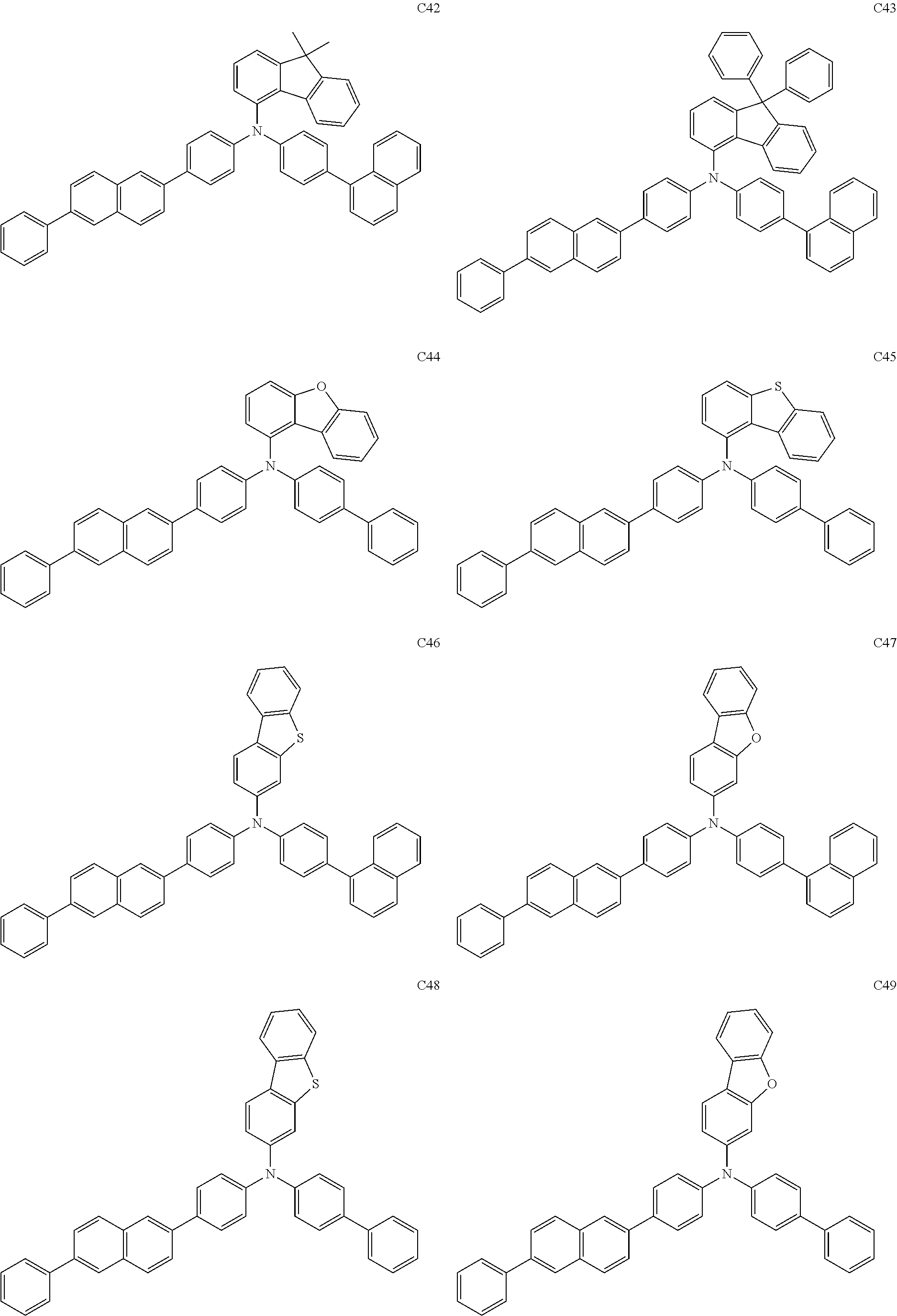 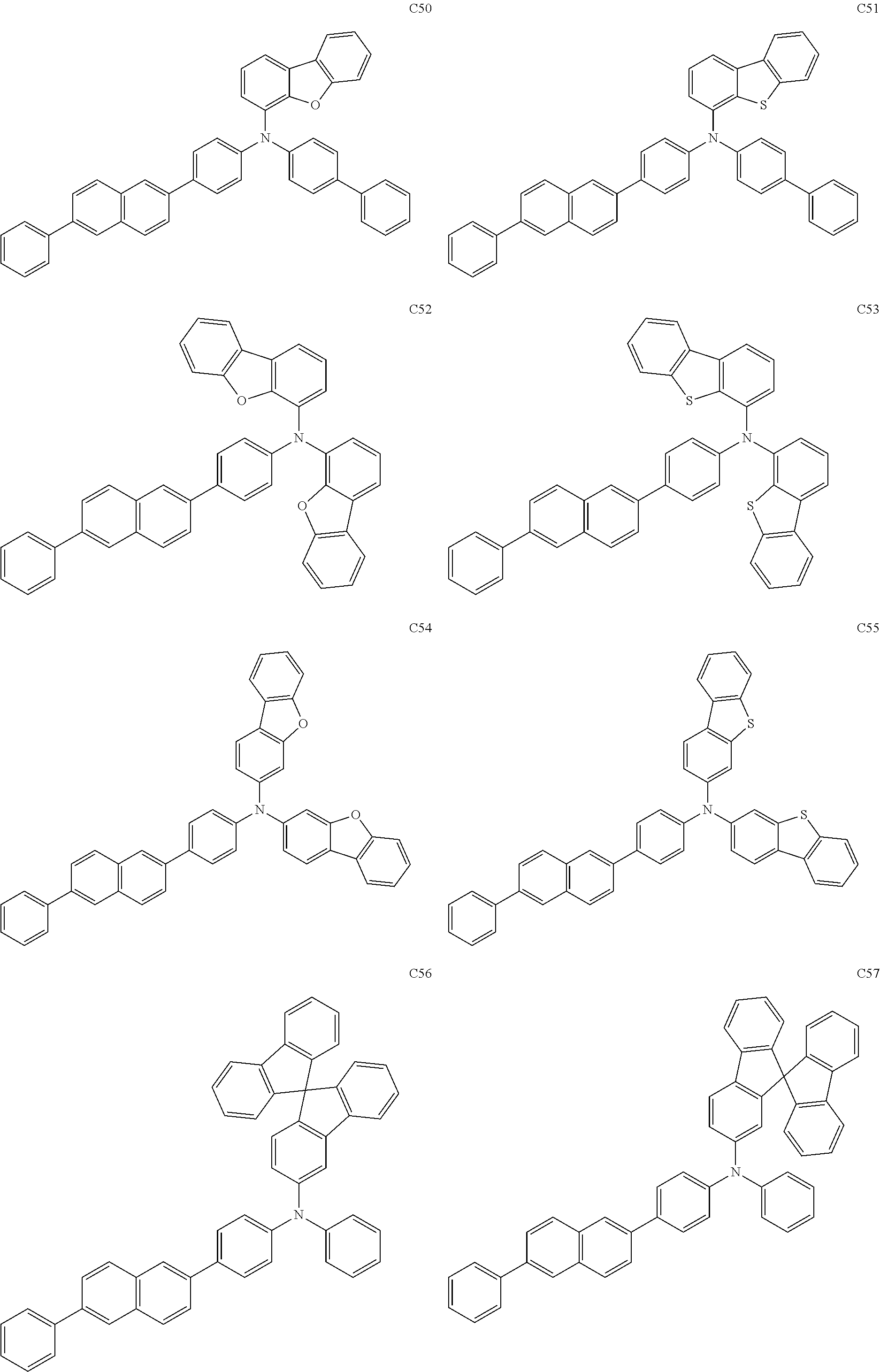 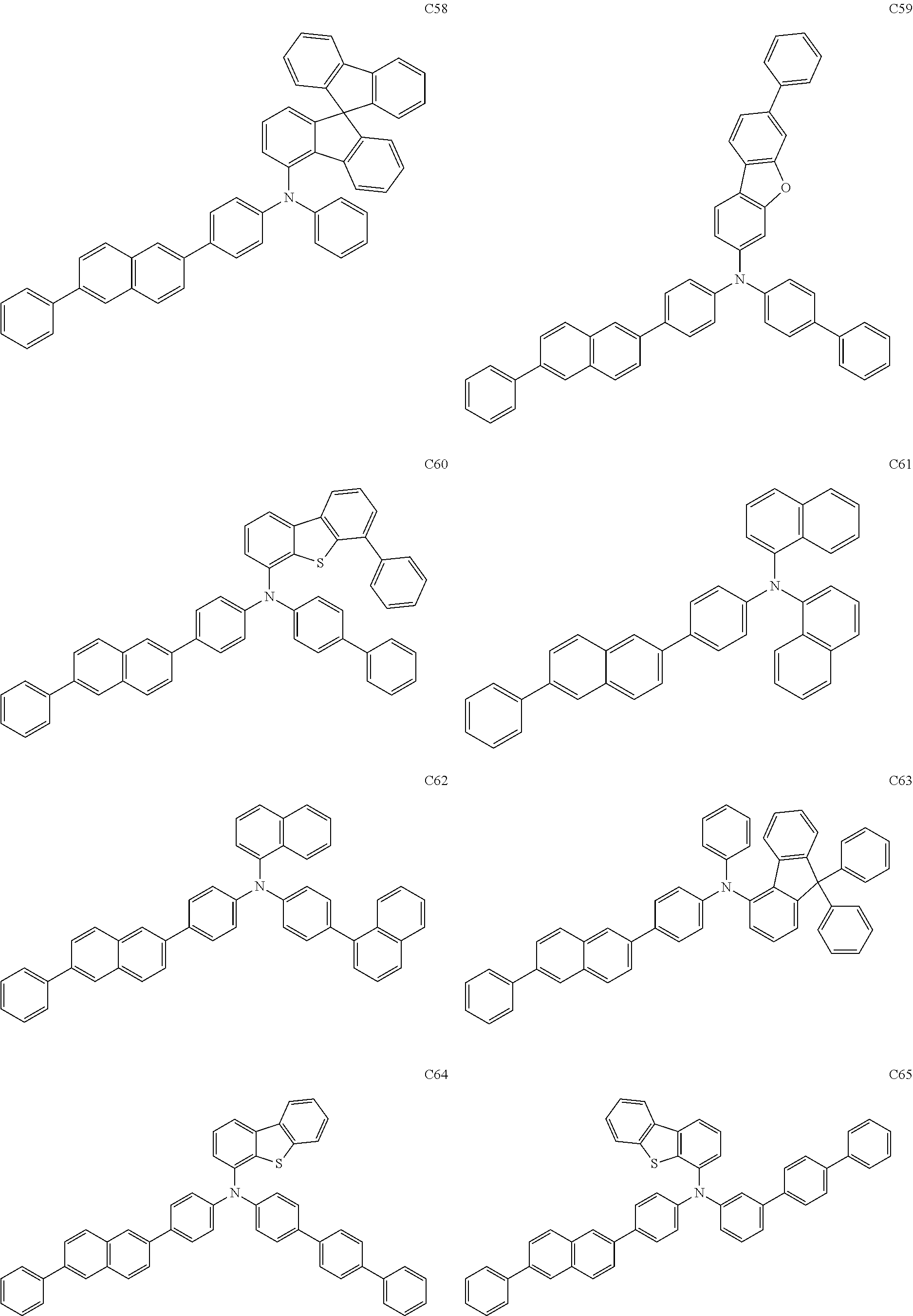 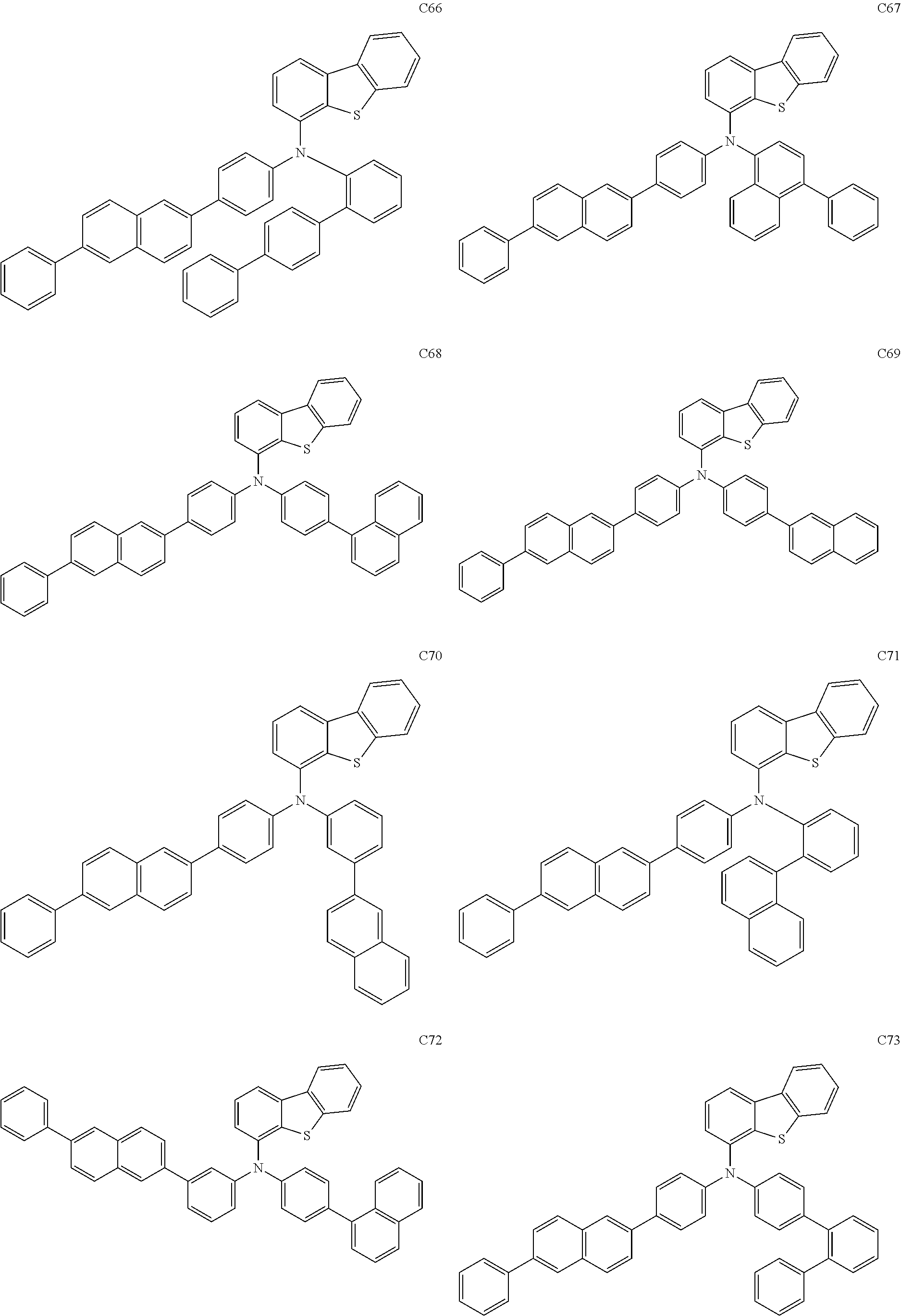 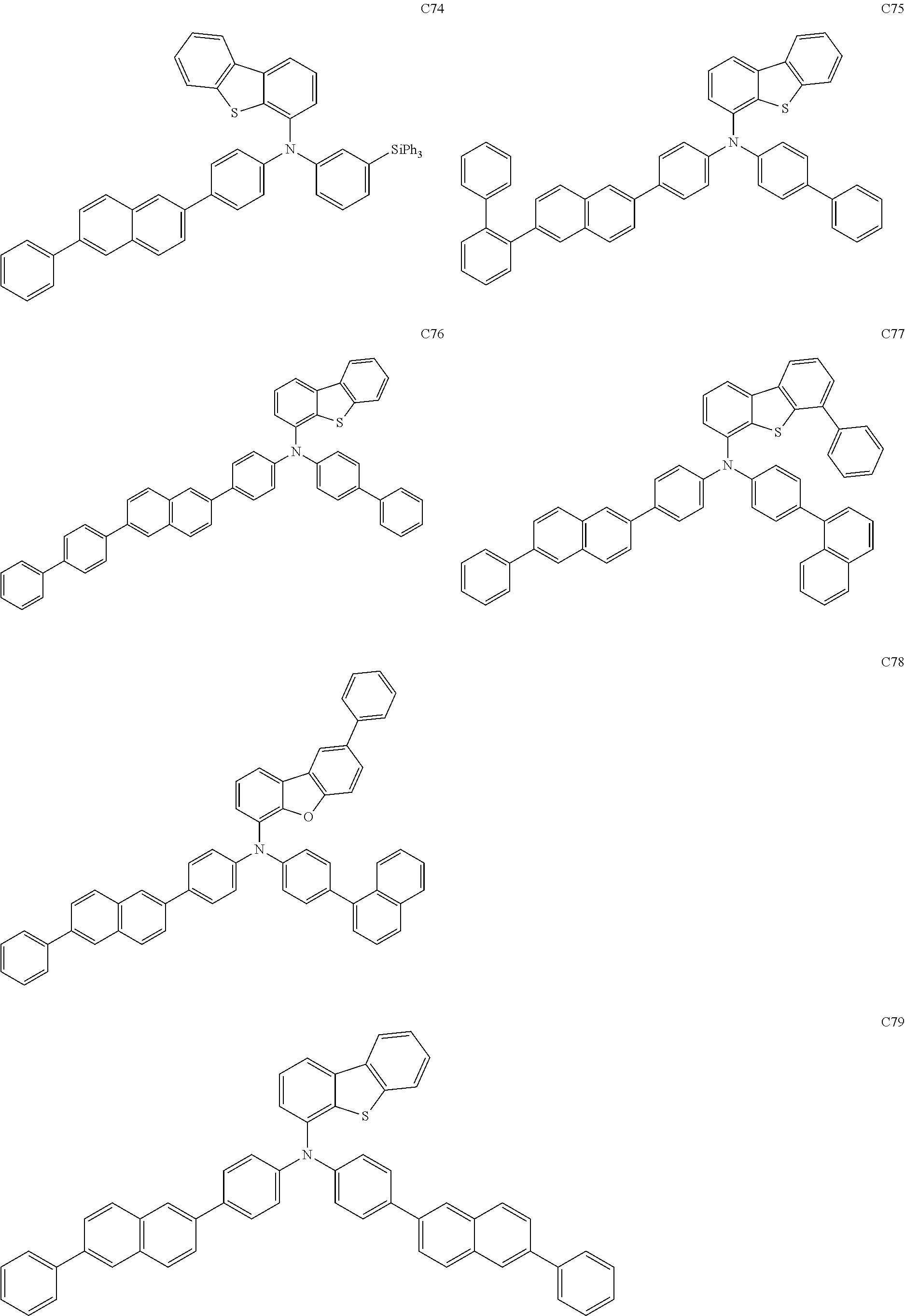 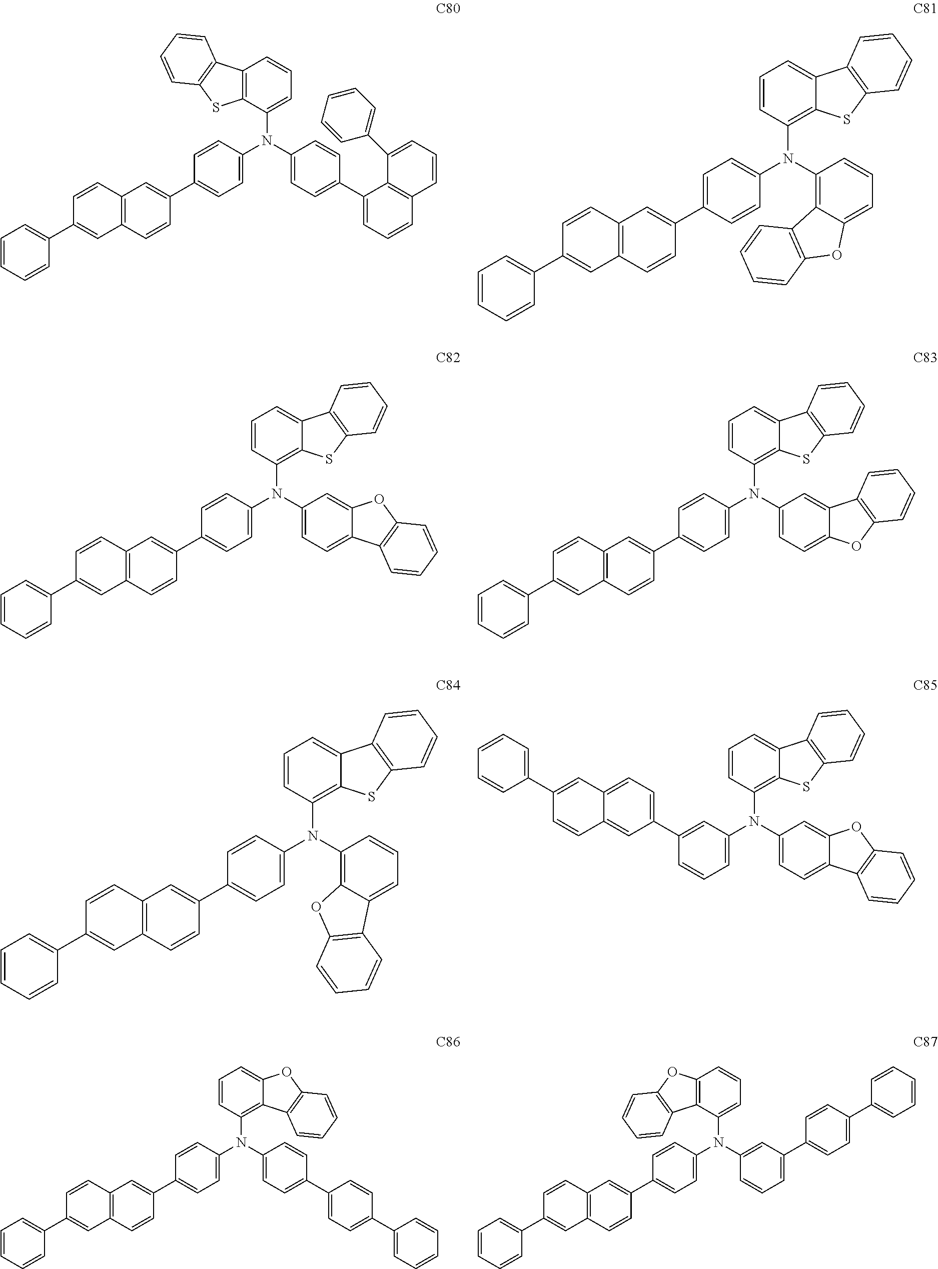 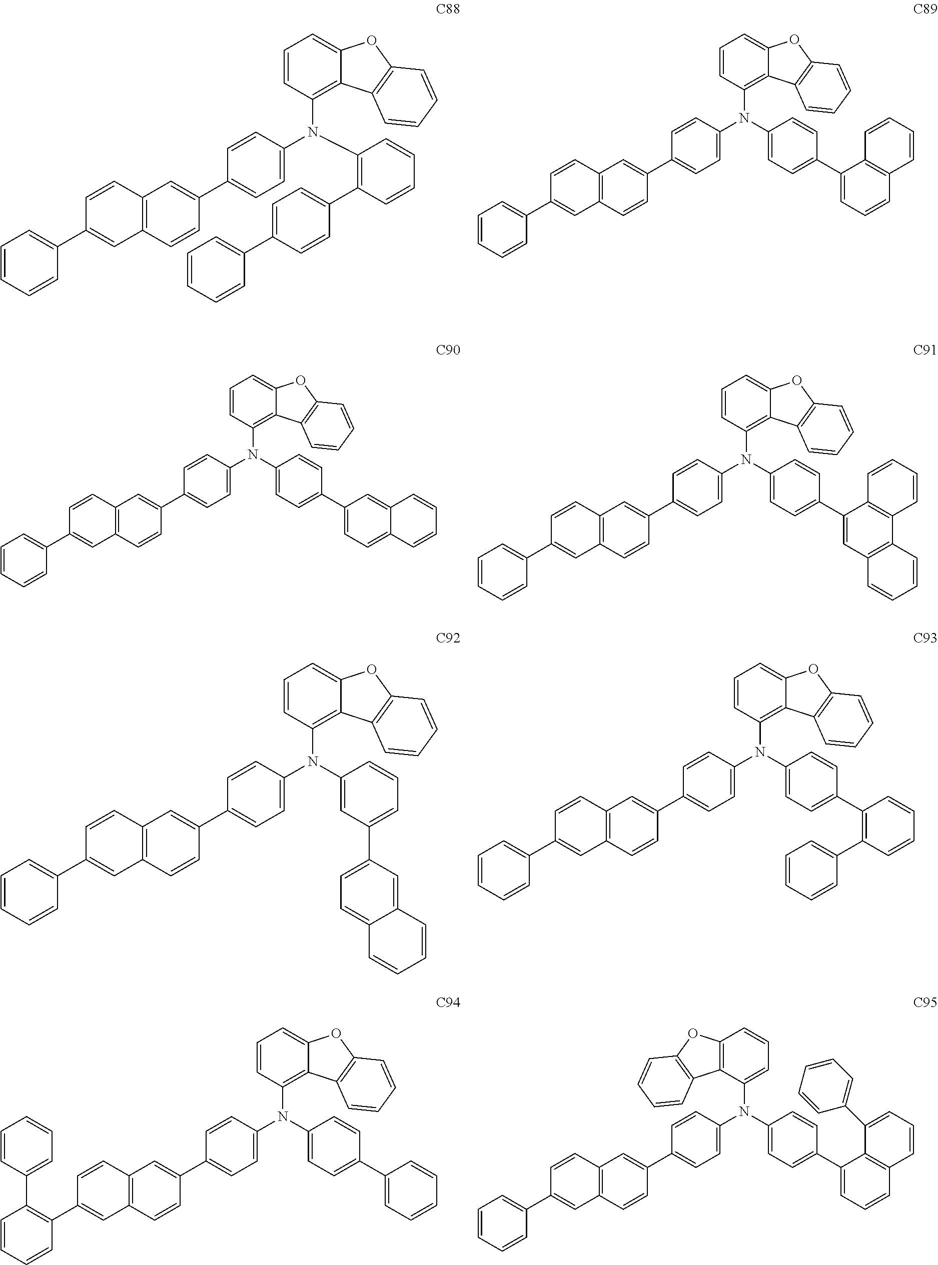 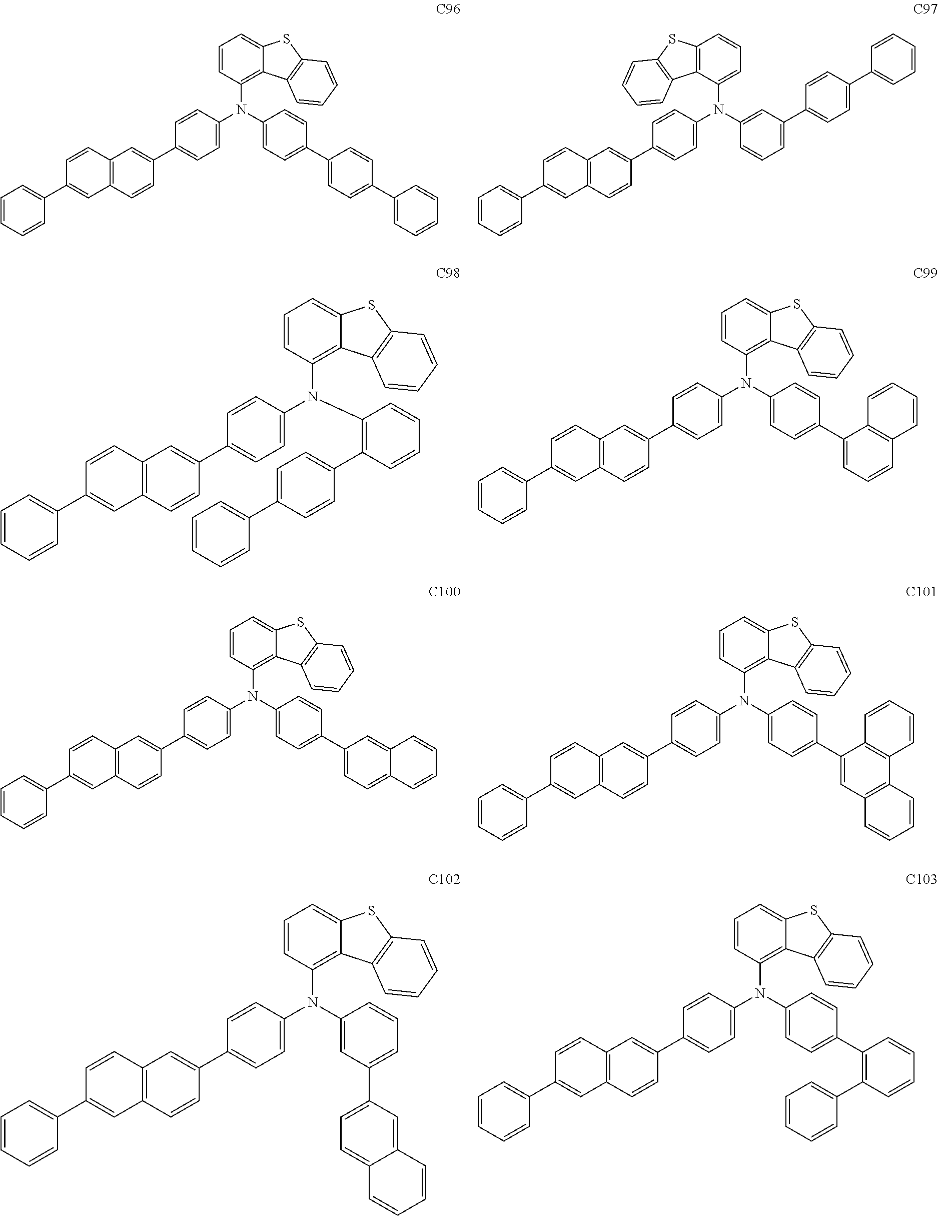 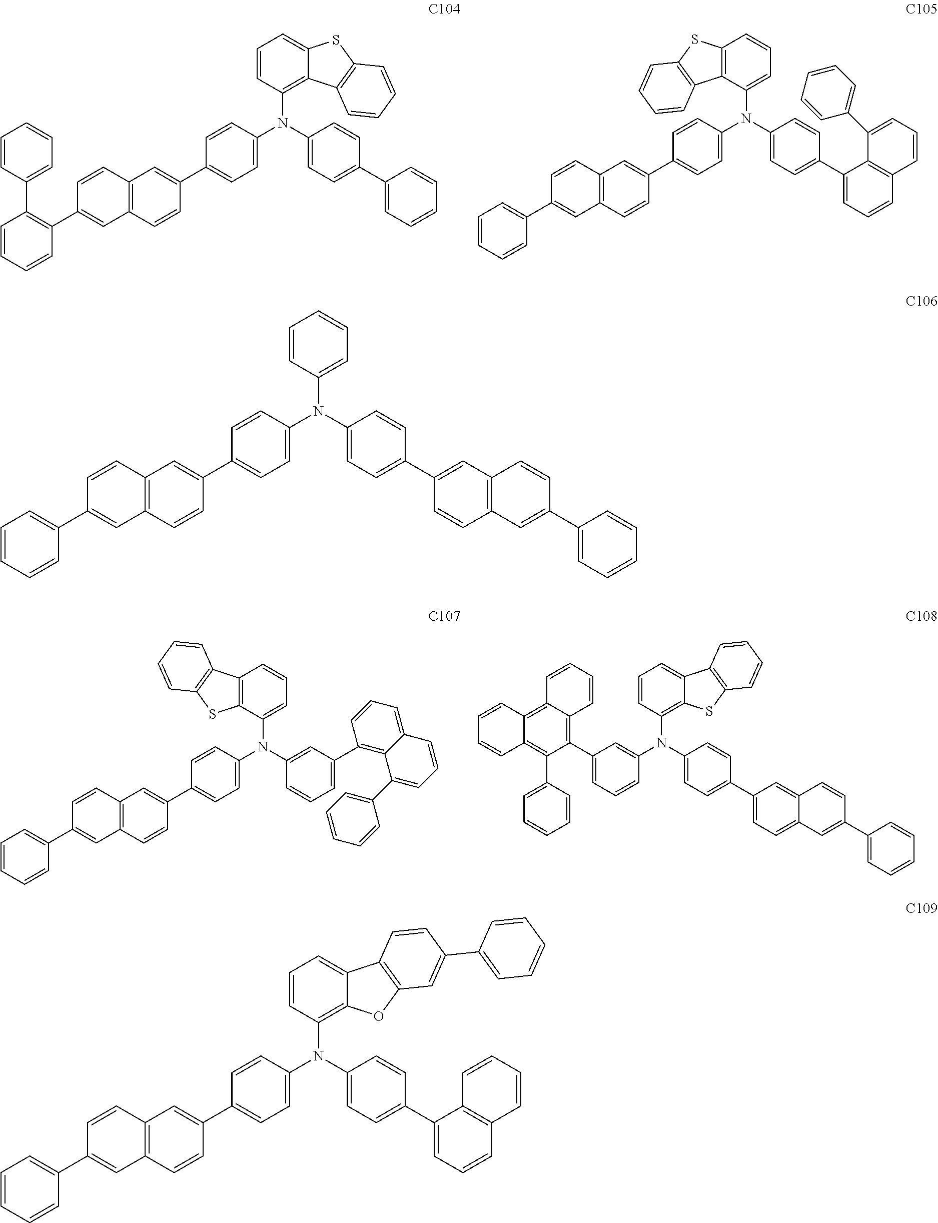 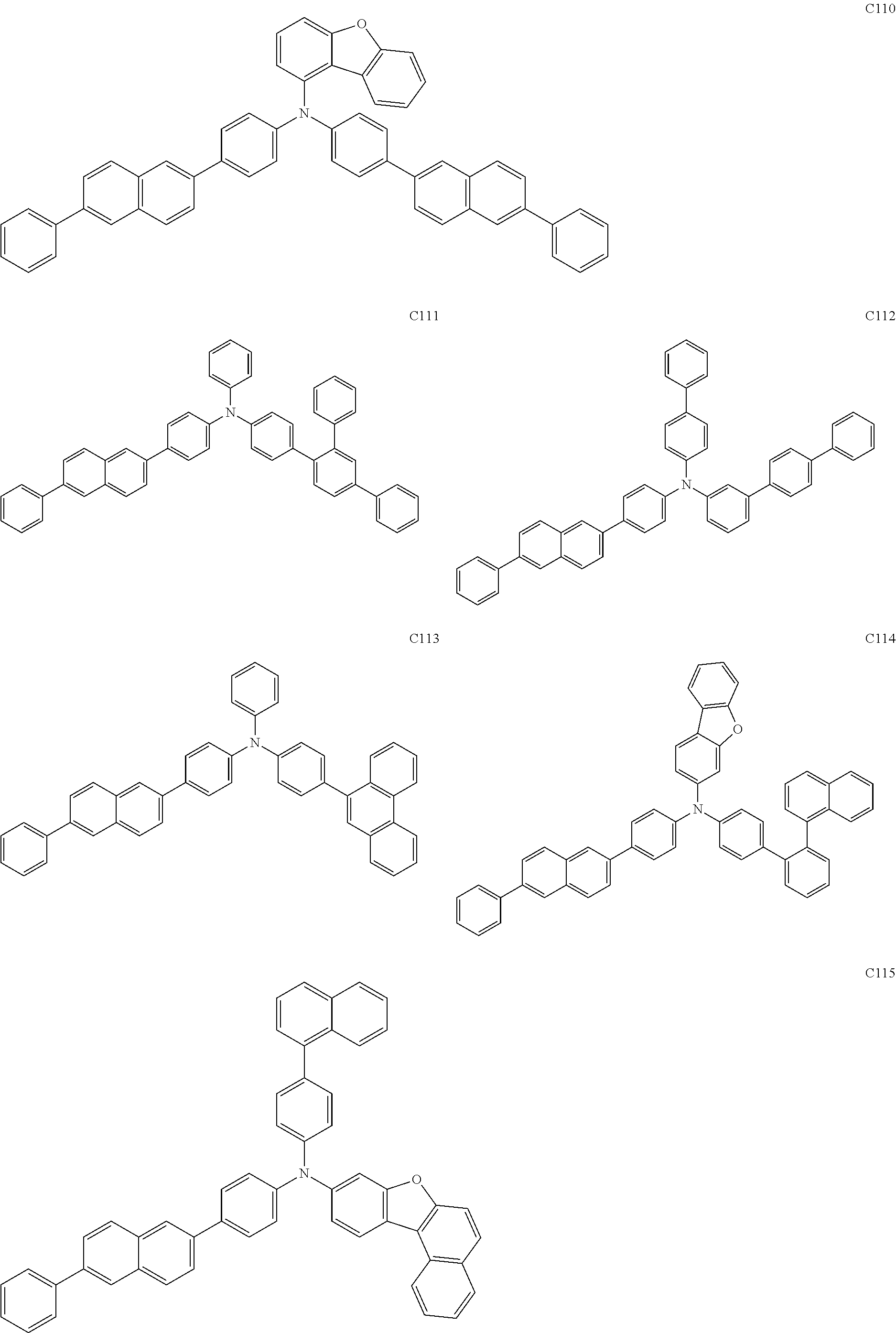 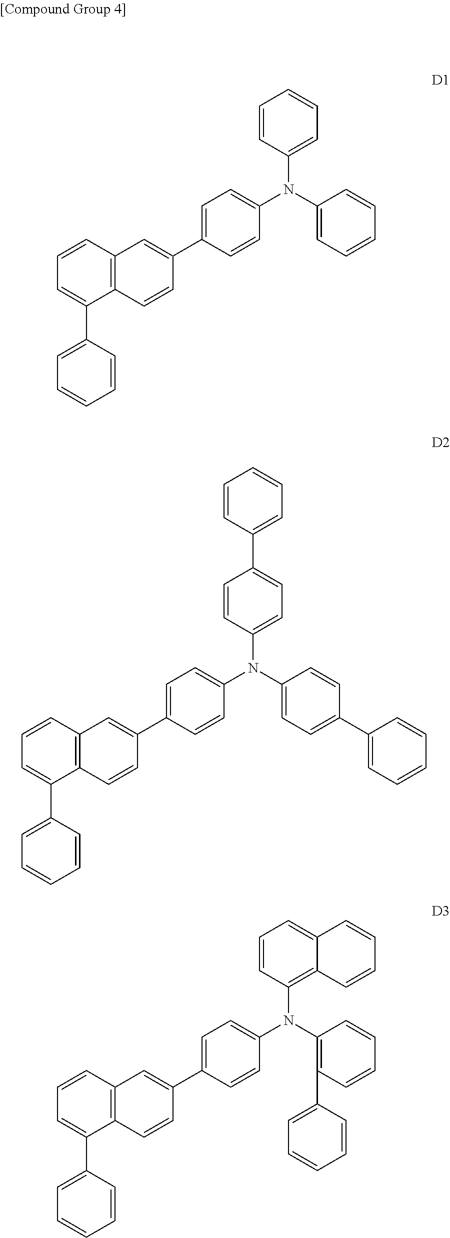 [SMILES: c1ccc(-c2cccc3cc(-c4ccc(N(c5ccccc5)c5ccccc5)cc4)ccc23)cc1 ; c1ccc(-c2ccc(N(c3ccc(-c4ccccc4)cc3)c3ccc(-c4ccc5c(-c6ccccc6)cccc5c4)cc3)cc2)cc1 ; c1ccc(-c2ccccc2N(c2ccc(-c3ccc4c(-c5ccccc5)cccc4c3)cc2)c2cccc3ccccc23)cc1] 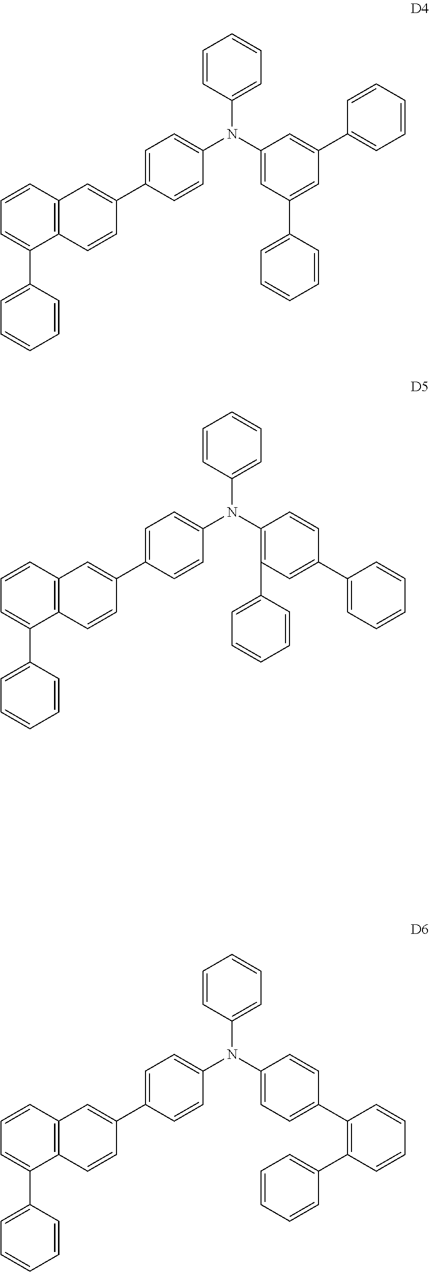 [SMILES: c1ccc(-c2cc(-c3ccccc3)cc(N(c3ccccc3)c3ccc(-c4ccc5c(-c6ccccc6)cccc5c4)cc3)c2)cc1 ; c1ccc(-c2ccc(N(c3ccccc3)c3ccc(-c4ccc5c(-c6ccccc6)cccc5c4)cc3)c(-c3ccccc3)c2)cc1 ; c1ccc(-c2ccccc2-c2ccc(N(c3ccccc3)c3ccc(-c4ccc5c(-c6ccccc6)cccc5c4)cc3)cc2)cc1] 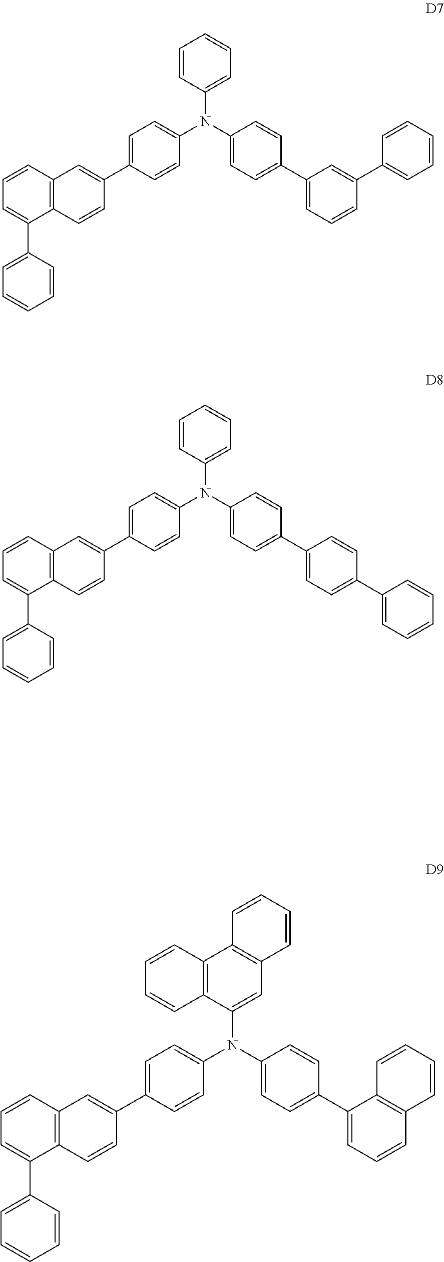 [SMILES: c1ccc(-c2cccc(-c3ccc(N(c4ccccc4)c4ccc(-c5ccc6c(-c7ccccc7)cccc6c5)cc4)cc3)c2)cc1 ; c1ccc(-c2ccc(-c3ccc(N(c4ccccc4)c4ccc(-c5ccc6c(-c7ccccc7)cccc6c5)cc4)cc3)cc2)cc1 ; c1ccc(-c2cccc3cc(-c4ccc(N(c5ccc(-c6cccc7ccccc67)cc5)c5cc6ccccc6c6ccccc56)cc4)ccc23)cc1] 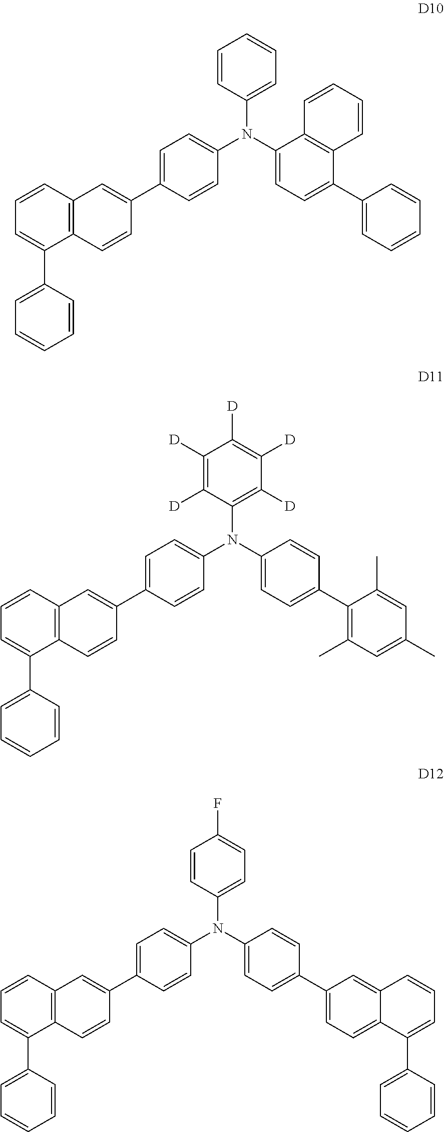 [SMILES: c1ccc(-c2cccc3cc(-c4ccc(N(c5ccccc5)c5ccc(-c6ccccc6)c6ccccc56)cc4)ccc23)cc1 ; [2H]c1c([2H])c([2H])c(N(c2ccc(-c3ccc4c(-c5ccccc5)cccc4c3)cc2)c2ccc(-c3c(C)cc(C)cc3C)cc2)c([2H])c1[2H] ; Fc1ccc(N(c2ccc(-c3ccc4c(-c5ccccc5)cccc4c3)cc2)c2ccc(-c3ccc4c(-c5ccccc5)cccc4c3)cc2)cc1] 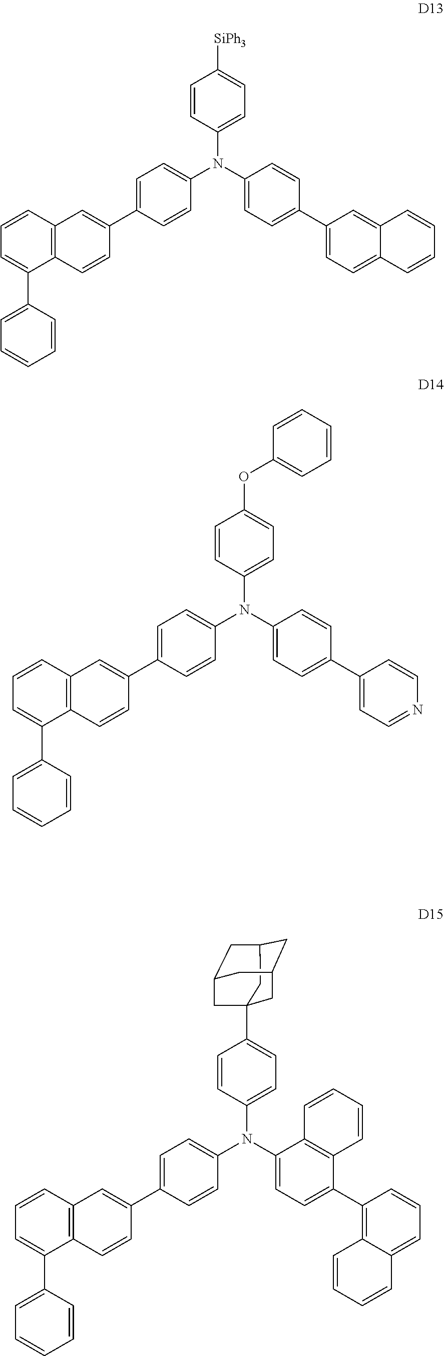 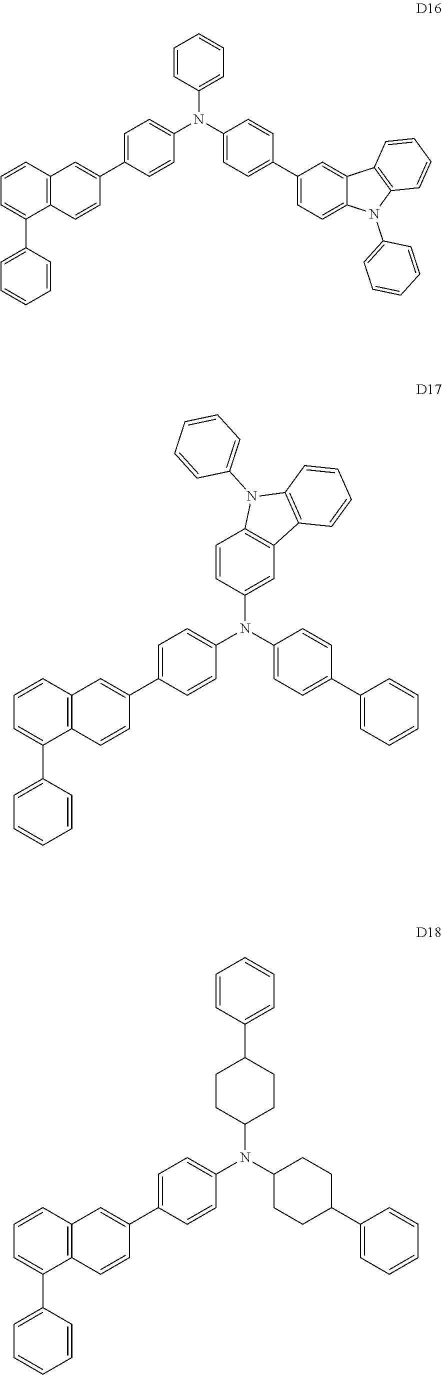 [SMILES: c1ccc(-c2cccc3cc(-c4ccc(N(c5ccccc5)c5ccc(-c6ccc7c(c6)c6ccccc6n7-c6ccccc6)cc5)cc4)ccc23)cc1 ; c1ccc(-c2ccc(N(c3ccc(-c4ccc5c(-c6ccccc6)cccc5c4)cc3)c3ccc4c(c3)c3ccccc3n4-c3ccccc3)cc2)cc1 ; c1ccc(-c2cccc3cc(-c4ccc(N(C5CCC(c6ccccc6)CC5)C5CCC(c6ccccc6)CC5)cc4)ccc23)cc1] 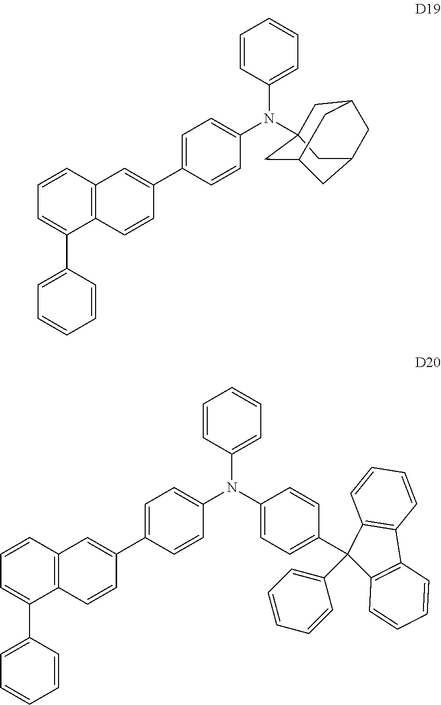 [SMILES: c1ccc(-c2cccc3cc(-c4ccc(N(c5ccccc5)C56CC7CC(CC(C7)C5)C6)cc4)ccc23)cc1 ; c1ccc(-c2cccc3cc(-c4ccc(N(c5ccccc5)c5ccc(C6(c7ccccc7)c7ccccc7-c7ccccc76)cc5)cc4)ccc23)cc1] 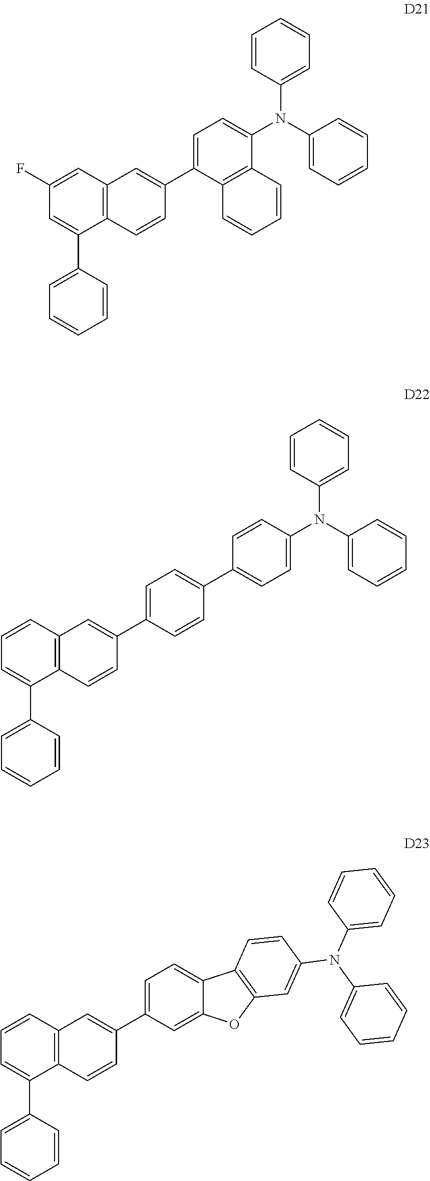 [SMILES: Fc1cc(-c2ccccc2)c2ccc(-c3ccc(N(c4ccccc4)c4ccccc4)c4ccccc34)cc2c1 ; c1ccc(-c2cccc3cc(-c4ccc(-c5ccc(N(c6ccccc6)c6ccccc6)cc5)cc4)ccc23)cc1 ; c1ccc(-c2cccc3cc(-c4ccc5c(c4)oc4cc(N(c6ccccc6)c6ccccc6)ccc45)ccc23)cc1] 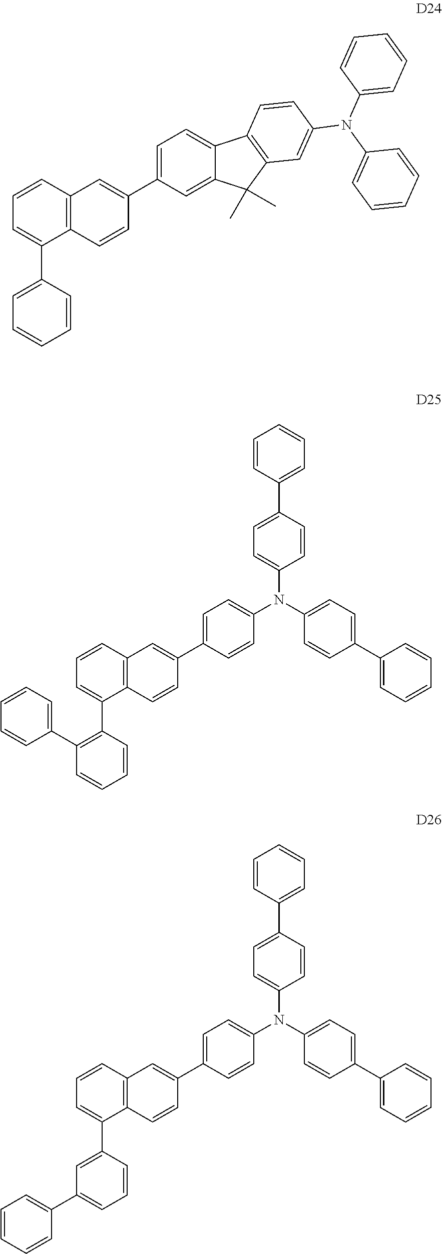 [SMILES: CC1(C)c2cc(-c3ccc4c(-c5ccccc5)cccc4c3)ccc2-c2ccc(N(c3ccccc3)c3ccccc3)cc21 ; c1ccc(-c2ccc(N(c3ccc(-c4ccccc4)cc3)c3ccc(-c4ccc5c(-c6ccccc6-c6ccccc6)cccc5c4)cc3)cc2)cc1 ; c1ccc(-c2ccc(N(c3ccc(-c4ccccc4)cc3)c3ccc(-c4ccc5c(-c6cccc(-c7ccccc7)c6)cccc5c4)cc3)cc2)cc1] 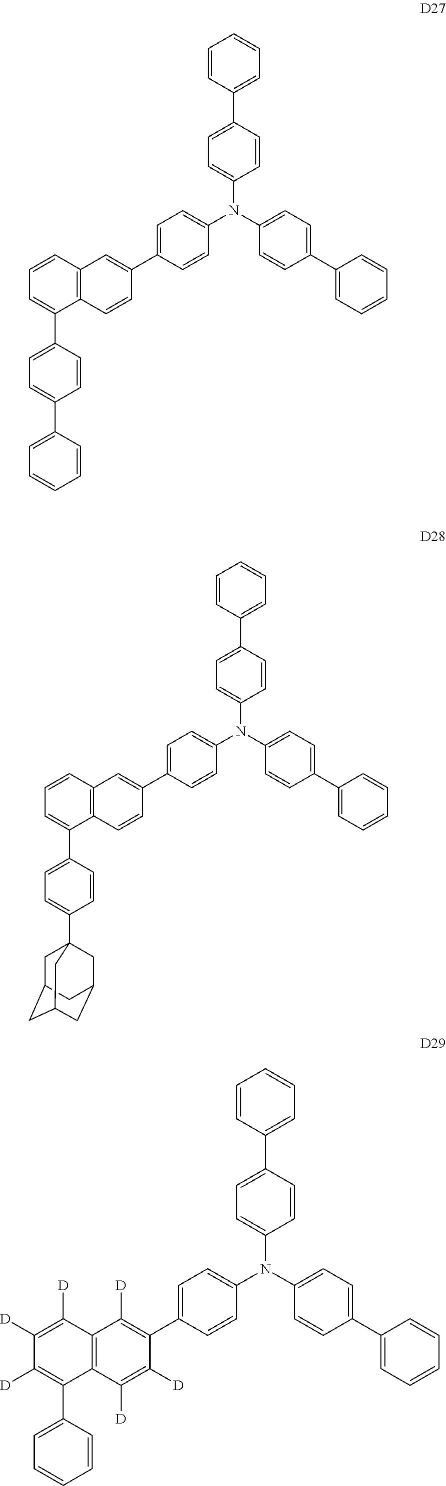 [SMILES: c1ccc(-c2ccc(-c3cccc4cc(-c5ccc(N(c6ccc(-c7ccccc7)cc6)c6ccc(-c7ccccc7)cc6)cc5)ccc34)cc2)cc1 ; c1ccc(-c2ccc(N(c3ccc(-c4ccccc4)cc3)c3ccc(-c4ccc5c(-c6ccc(C78CC9CC(CC(C9)C7)C8)cc6)cccc5c4)cc3)cc2)cc1 ; [2H]c1c([2H])c(-c2ccccc2)c2c([2H])c([2H])c(-c3ccc(N(c4ccc(-c5ccccc5)cc4)c4ccc(-c5ccccc5)cc4)cc3)c([2H])c2c1[2H]] 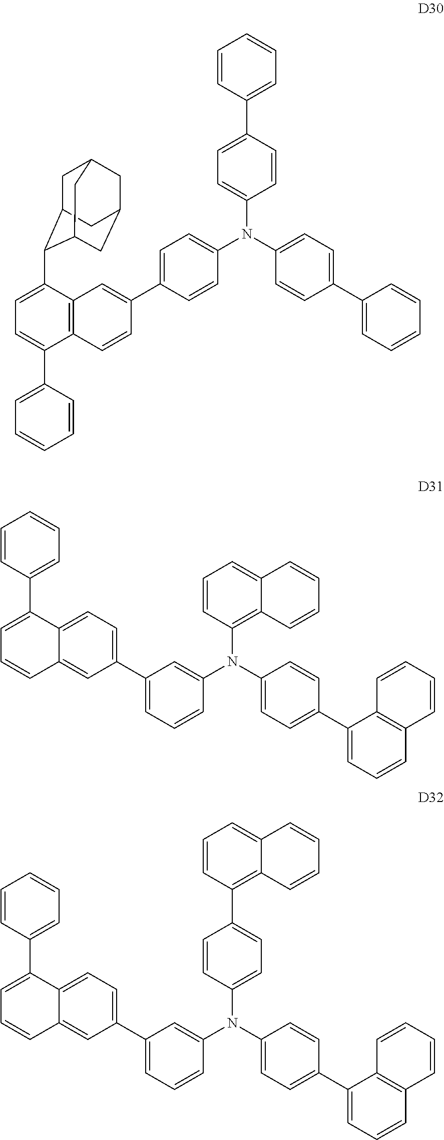 [SMILES: c1ccc(-c2ccc(N(c3ccc(-c4ccccc4)cc3)c3ccc(-c4ccc5c(-c6ccccc6)ccc(C6C7CC8CC(C7)CC6C8)c5c4)cc3)cc2)cc1 ; c1ccc(-c2cccc3cc(-c4cccc(N(c5ccc(-c6cccc7ccccc67)cc5)c5cccc6ccccc56)c4)ccc23)cc1 ; c1ccc(-c2cccc3cc(-c4cccc(N(c5ccc(-c6cccc7ccccc67)cc5)c5ccc(-c6cccc7ccccc67)cc5)c4)ccc23)cc1] 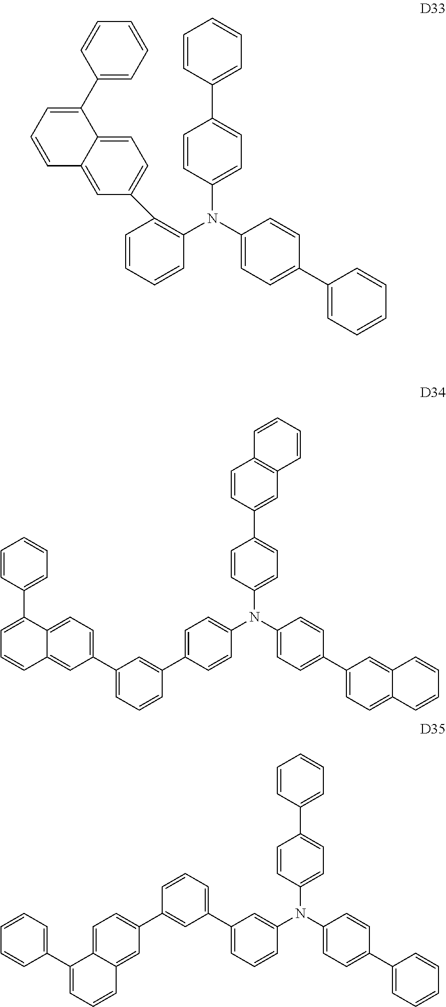 [SMILES: c1ccc(-c2ccc(N(c3ccc(-c4ccccc4)cc3)c3ccccc3-c3ccc4c(-c5ccccc5)cccc4c3)cc2)cc1 ; c1ccc(-c2cccc3cc(-c4cccc(-c5ccc(N(c6ccc(-c7ccc8ccccc8c7)cc6)c6ccc(-c7ccc8ccccc8c7)cc6)cc5)c4)ccc23)cc1 ; c1ccc(-c2ccc(N(c3ccc(-c4ccccc4)cc3)c3cccc(-c4cccc(-c5ccc6c(-c7ccccc7)cccc6c5)c4)c3)cc2)cc1] 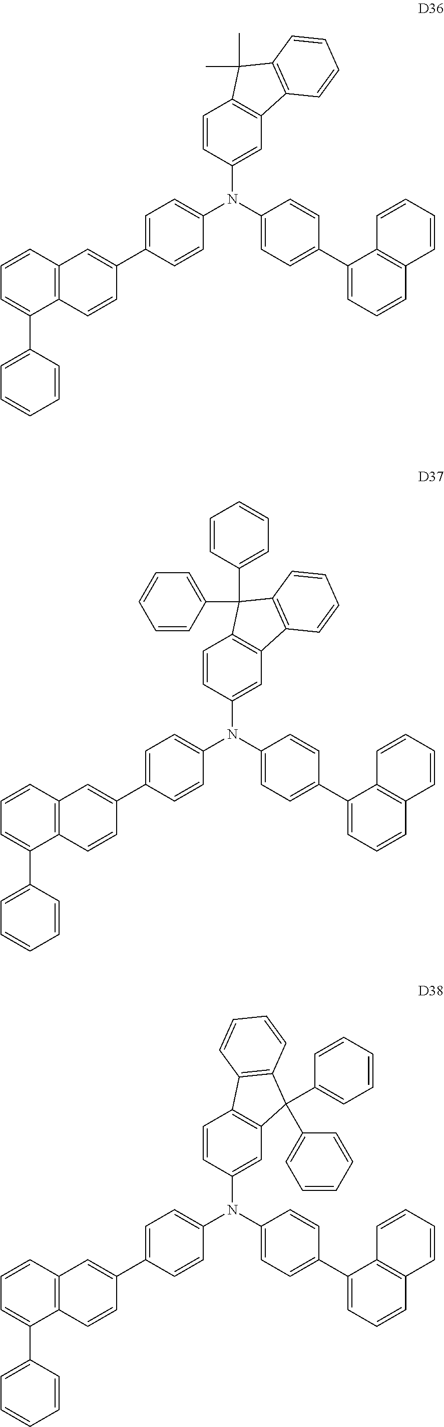 [SMILES: CC1(C)c2ccccc2-c2cc(N(c3ccc(-c4ccc5c(-c6ccccc6)cccc5c4)cc3)c3ccc(-c4cccc5ccccc45)cc3)ccc21 ; c1ccc(-c2cccc3cc(-c4ccc(N(c5ccc(-c6cccc7ccccc67)cc5)c5ccc6c(c5)-c5ccccc5C6(c5ccccc5)c5ccccc5)cc4)ccc23)cc1 ; c1ccc(-c2cccc3cc(-c4ccc(N(c5ccc(-c6cccc7ccccc67)cc5)c5ccc6c(c5)C(c5ccccc5)(c5ccccc5)c5ccccc5-6)cc4)ccc23)cc1] 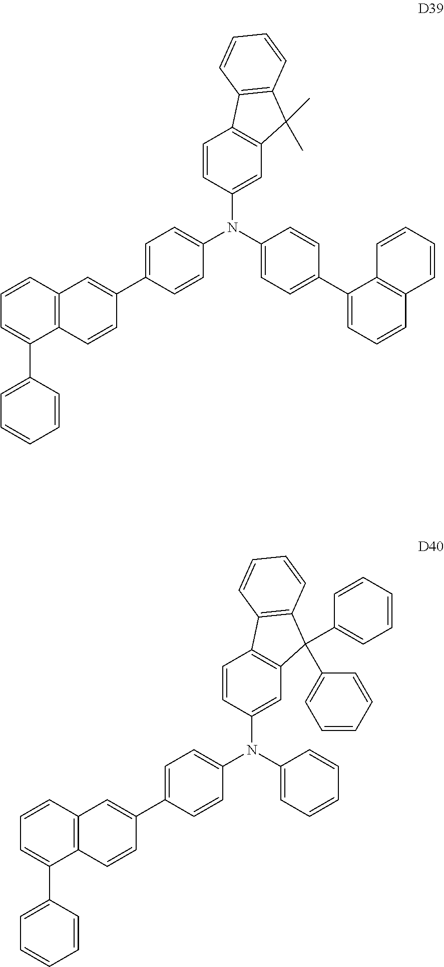 [SMILES: CC1(C)c2ccccc2-c2ccc(N(c3ccc(-c4ccc5c(-c6ccccc6)cccc5c4)cc3)c3ccc(-c4cccc5ccccc45)cc3)cc21 ; c1ccc(-c2cccc3cc(-c4ccc(N(c5ccccc5)c5ccc6c(c5)C(c5ccccc5)(c5ccccc5)c5ccccc5-6)cc4)ccc23)cc1] 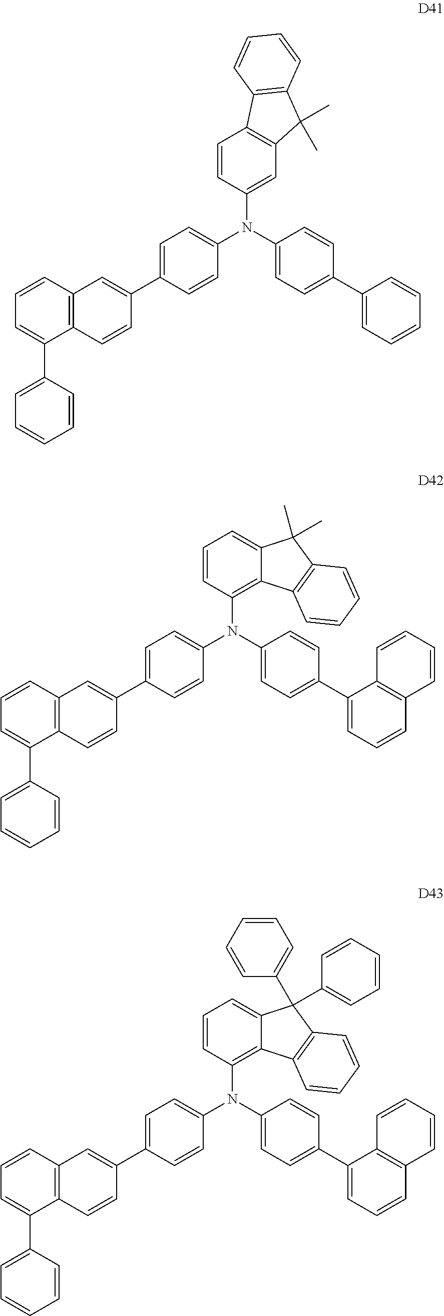 [SMILES: CC1(C)c2ccccc2-c2ccc(N(c3ccc(-c4ccccc4)cc3)c3ccc(-c4ccc5c(-c6ccccc6)cccc5c4)cc3)cc21 ; CC1(C)c2ccccc2-c2c(N(c3ccc(-c4ccc5c(-c6ccccc6)cccc5c4)cc3)c3ccc(-c4cccc5ccccc45)cc3)cccc21 ; c1ccc(-c2cccc3cc(-c4ccc(N(c5ccc(-c6cccc7ccccc67)cc5)c5cccc6c5-c5ccccc5C6(c5ccccc5)c5ccccc5)cc4)ccc23)cc1] 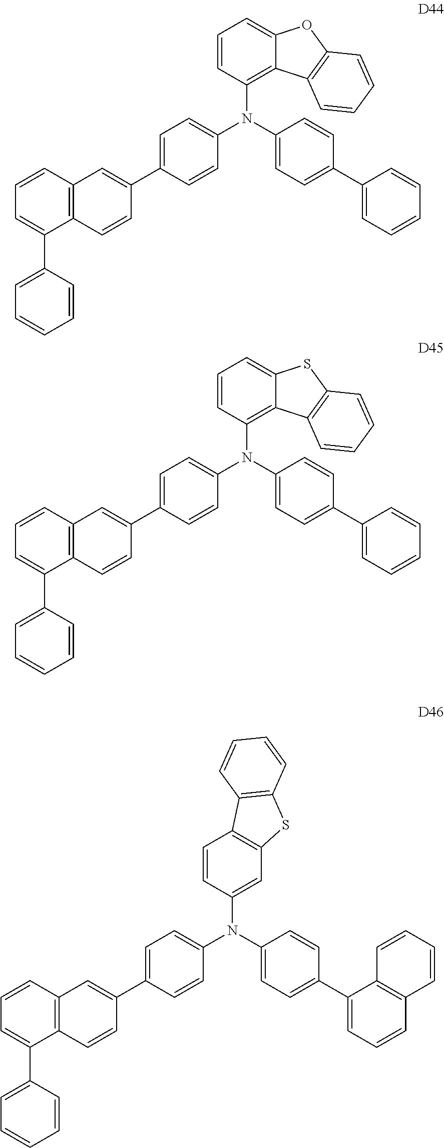 [SMILES: c1ccc(-c2ccc(N(c3ccc(-c4ccc5c(-c6ccccc6)cccc5c4)cc3)c3cccc4oc5ccccc5c34)cc2)cc1 ; c1ccc(-c2ccc(N(c3ccc(-c4ccc5c(-c6ccccc6)cccc5c4)cc3)c3cccc4sc5ccccc5c34)cc2)cc1 ; c1ccc(-c2cccc3cc(-c4ccc(N(c5ccc(-c6cccc7ccccc67)cc5)c5ccc6c(c5)sc5ccccc56)cc4)ccc23)cc1] 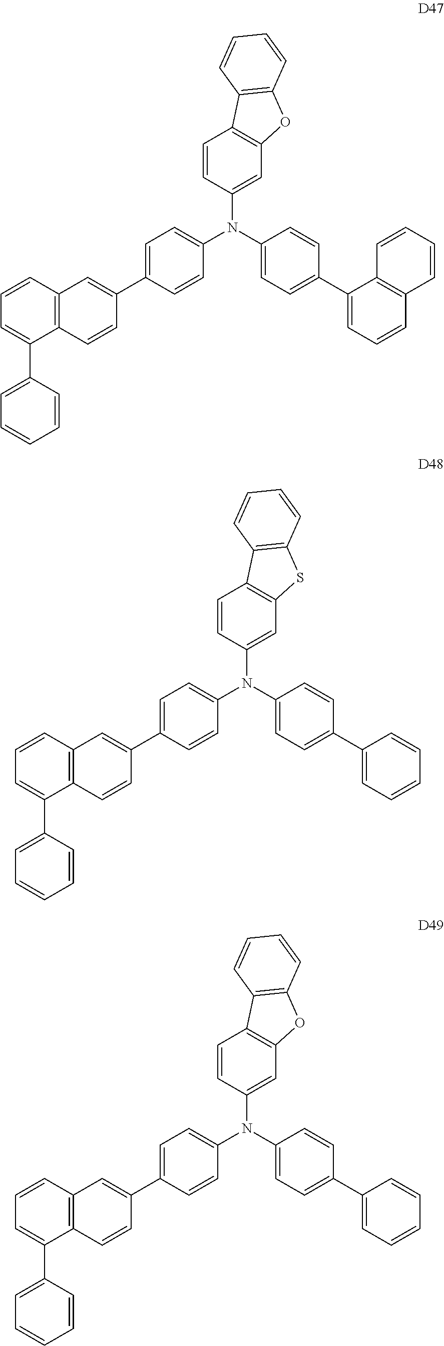 [SMILES: c1ccc(-c2cccc3cc(-c4ccc(N(c5ccc(-c6cccc7ccccc67)cc5)c5ccc6c(c5)oc5ccccc56)cc4)ccc23)cc1 ; c1ccc(-c2ccc(N(c3ccc(-c4ccc5c(-c6ccccc6)cccc5c4)cc3)c3ccc4c(c3)sc3ccccc34)cc2)cc1 ; c1ccc(-c2ccc(N(c3ccc(-c4ccc5c(-c6ccccc6)cccc5c4)cc3)c3ccc4c(c3)oc3ccccc34)cc2)cc1] 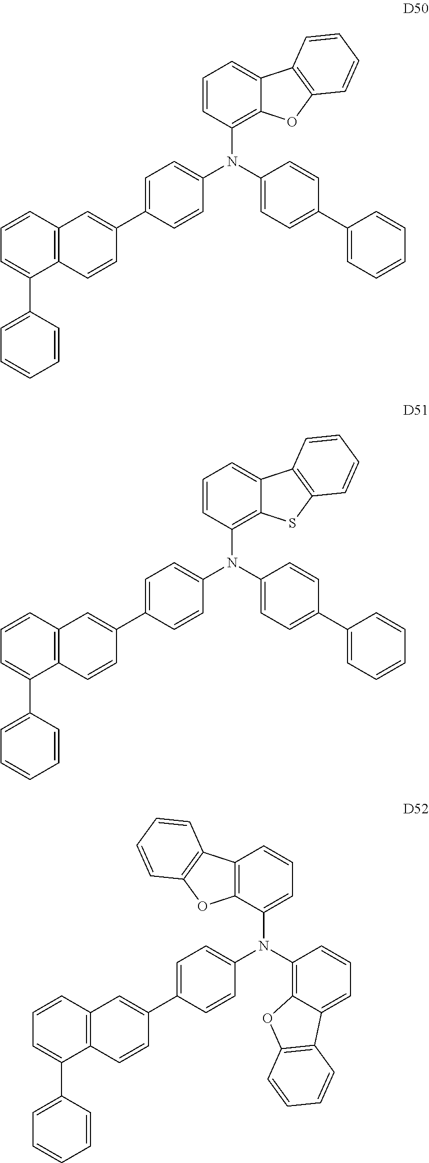 [SMILES: c1ccc(-c2ccc(N(c3ccc(-c4ccc5c(-c6ccccc6)cccc5c4)cc3)c3cccc4c3oc3ccccc34)cc2)cc1 ; c1ccc(-c2ccc(N(c3ccc(-c4ccc5c(-c6ccccc6)cccc5c4)cc3)c3cccc4c3sc3ccccc34)cc2)cc1 ; c1ccc(-c2cccc3cc(-c4ccc(N(c5cccc6c5oc5ccccc56)c5cccc6c5oc5ccccc56)cc4)ccc23)cc1] 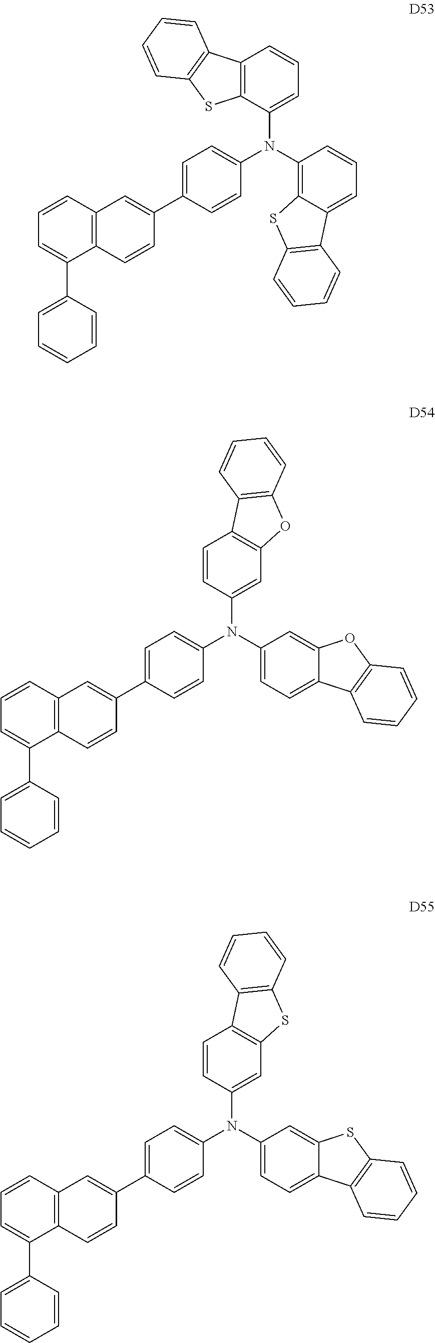 [SMILES: c1ccc(-c2cccc3cc(-c4ccc(N(c5cccc6c5sc5ccccc56)c5cccc6c5sc5ccccc56)cc4)ccc23)cc1 ; c1ccc(-c2cccc3cc(-c4ccc(N(c5ccc6c(c5)oc5ccccc56)c5ccc6c(c5)oc5ccccc56)cc4)ccc23)cc1 ; c1ccc(-c2cccc3cc(-c4ccc(N(c5ccc6c(c5)sc5ccccc56)c5ccc6c(c5)sc5ccccc56)cc4)ccc23)cc1] 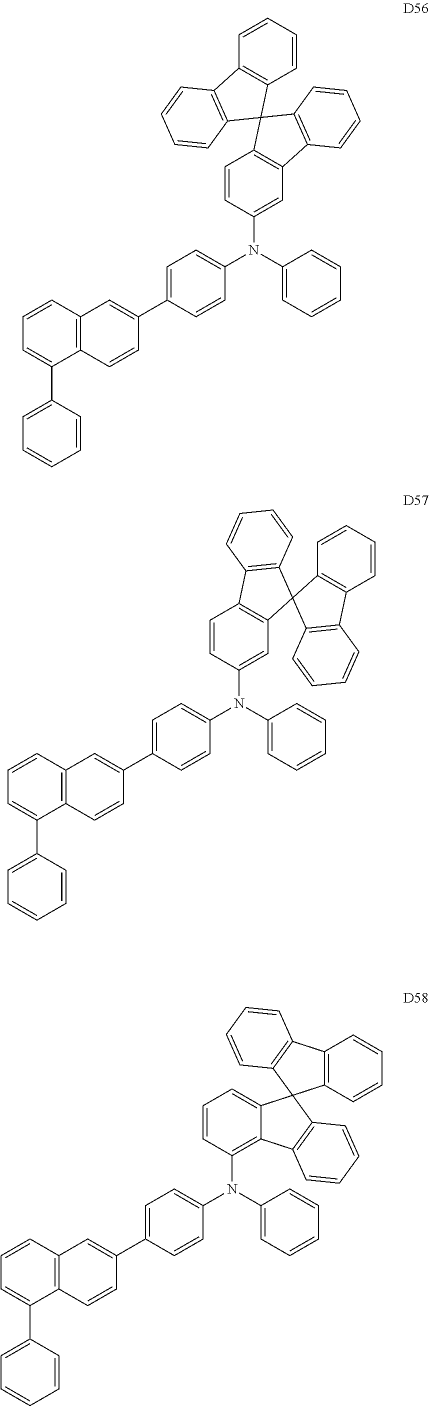 [SMILES: c1ccc(-c2cccc3cc(-c4ccc(N(c5ccccc5)c5ccc6c(c5)-c5ccccc5C65c6ccccc6-c6ccccc65)cc4)ccc23)cc1 ; c1ccc(-c2cccc3cc(-c4ccc(N(c5ccccc5)c5ccc6c(c5)C5(c7ccccc7-c7ccccc75)c5ccccc5-6)cc4)ccc23)cc1 ; c1ccc(-c2cccc3cc(-c4ccc(N(c5ccccc5)c5cccc6c5-c5ccccc5C65c6ccccc6-c6ccccc65)cc4)ccc23)cc1] 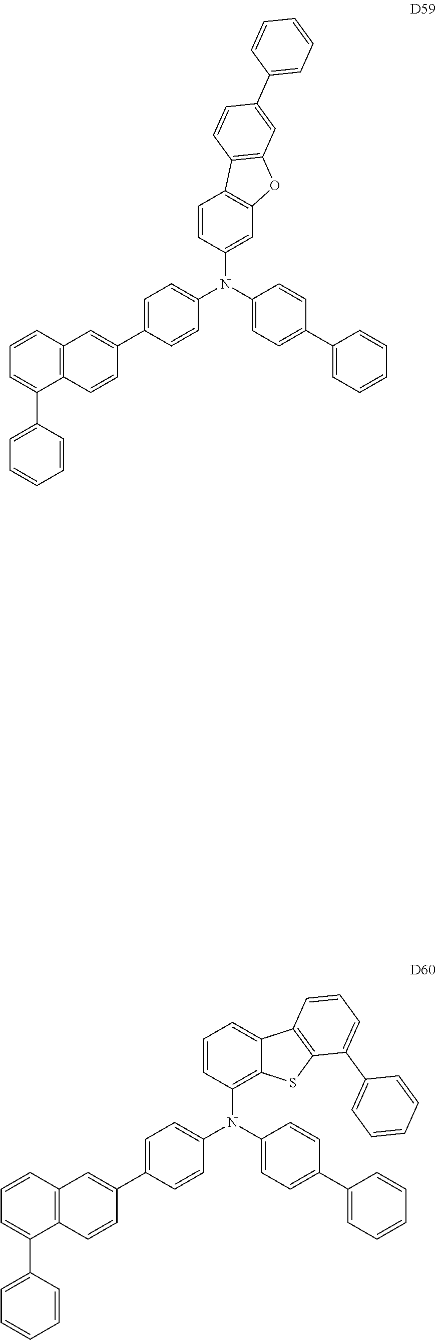 [SMILES: c1ccc(-c2ccc(N(c3ccc(-c4ccc5c(-c6ccccc6)cccc5c4)cc3)c3ccc4c(c3)oc3cc(-c5ccccc5)ccc34)cc2)cc1 ; c1ccc(-c2ccc(N(c3ccc(-c4ccc5c(-c6ccccc6)cccc5c4)cc3)c3cccc4c3sc3c(-c5ccccc5)cccc34)cc2)cc1] 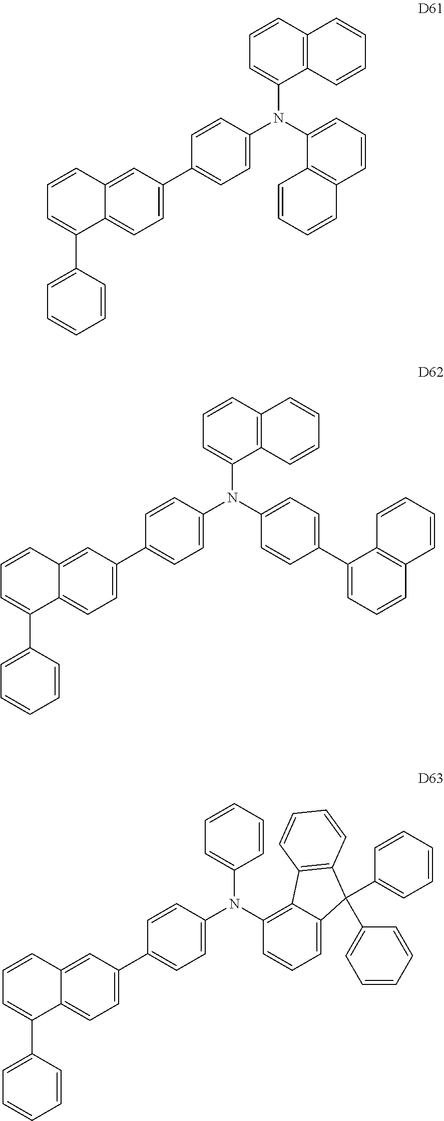 [SMILES: c1ccc(-c2cccc3cc(-c4ccc(N(c5cccc6ccccc56)c5cccc6ccccc56)cc4)ccc23)cc1 ; c1ccc(-c2cccc3cc(-c4ccc(N(c5ccc(-c6cccc7ccccc67)cc5)c5cccc6ccccc56)cc4)ccc23)cc1 ; c1ccc(-c2cccc3cc(-c4ccc(N(c5ccccc5)c5cccc6c5-c5ccccc5C6(c5ccccc5)c5ccccc5)cc4)ccc23)cc1] 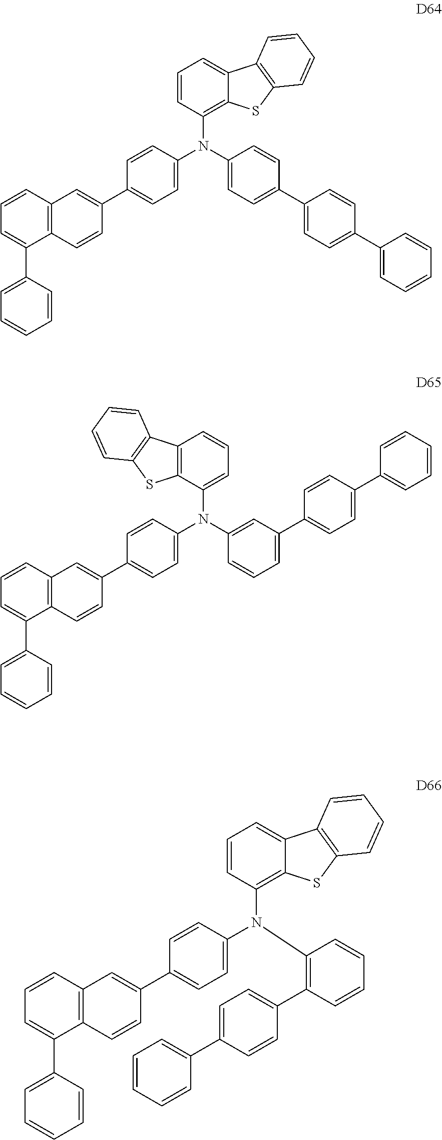 [SMILES: c1ccc(-c2ccc(-c3ccc(N(c4ccc(-c5ccc6c(-c7ccccc7)cccc6c5)cc4)c4cccc5c4sc4ccccc45)cc3)cc2)cc1 ; c1ccc(-c2ccc(-c3cccc(N(c4ccc(-c5ccc6c(-c7ccccc7)cccc6c5)cc4)c4cccc5c4sc4ccccc45)c3)cc2)cc1 ; c1ccc(-c2ccc(-c3ccccc3N(c3ccc(-c4ccc5c(-c6ccccc6)cccc5c4)cc3)c3cccc4c3sc3ccccc34)cc2)cc1] 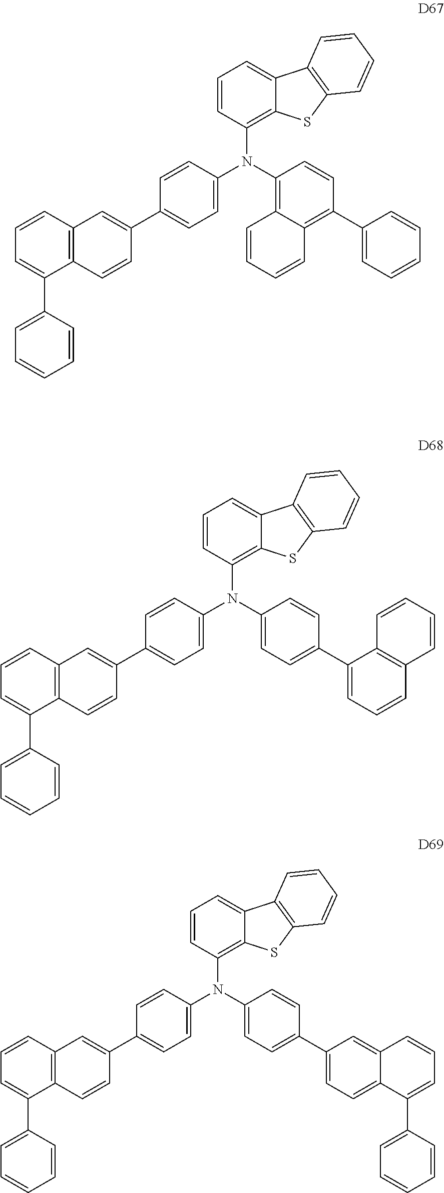 [SMILES: c1ccc(-c2cccc3cc(-c4ccc(N(c5ccc(-c6ccccc6)c6ccccc56)c5cccc6c5sc5ccccc56)cc4)ccc23)cc1 ; c1ccc(-c2cccc3cc(-c4ccc(N(c5ccc(-c6cccc7ccccc67)cc5)c5cccc6c5sc5ccccc56)cc4)ccc23)cc1 ; c1ccc(-c2cccc3cc(-c4ccc(N(c5ccc(-c6ccc7c(-c8ccccc8)cccc7c6)cc5)c5cccc6c5sc5ccccc56)cc4)ccc23)cc1] 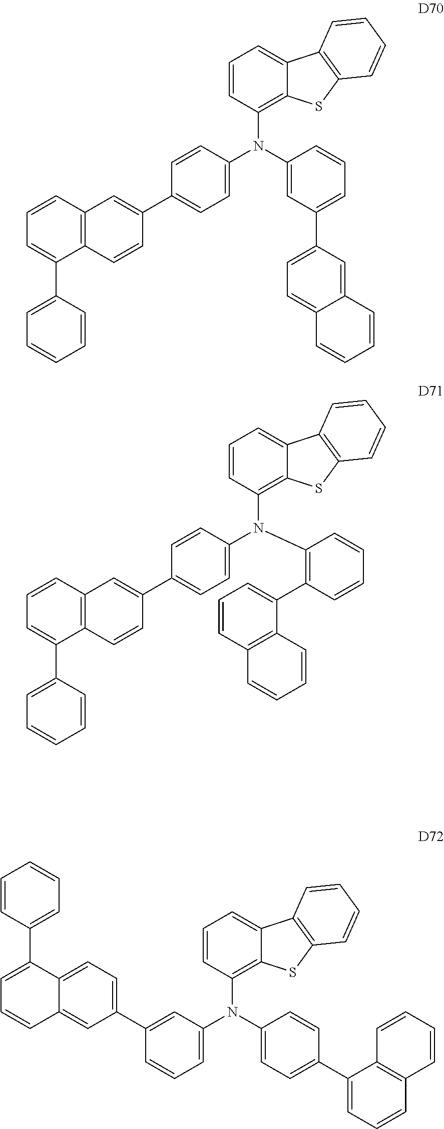 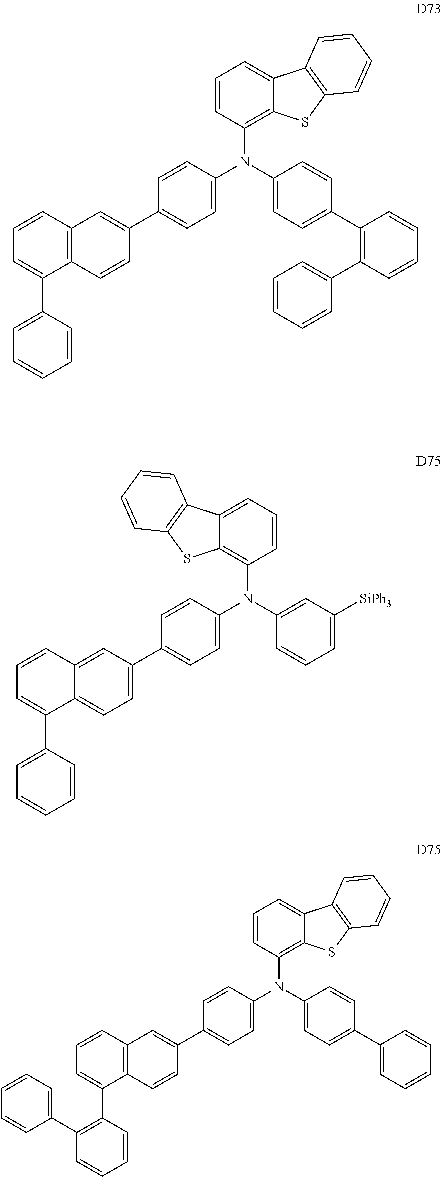 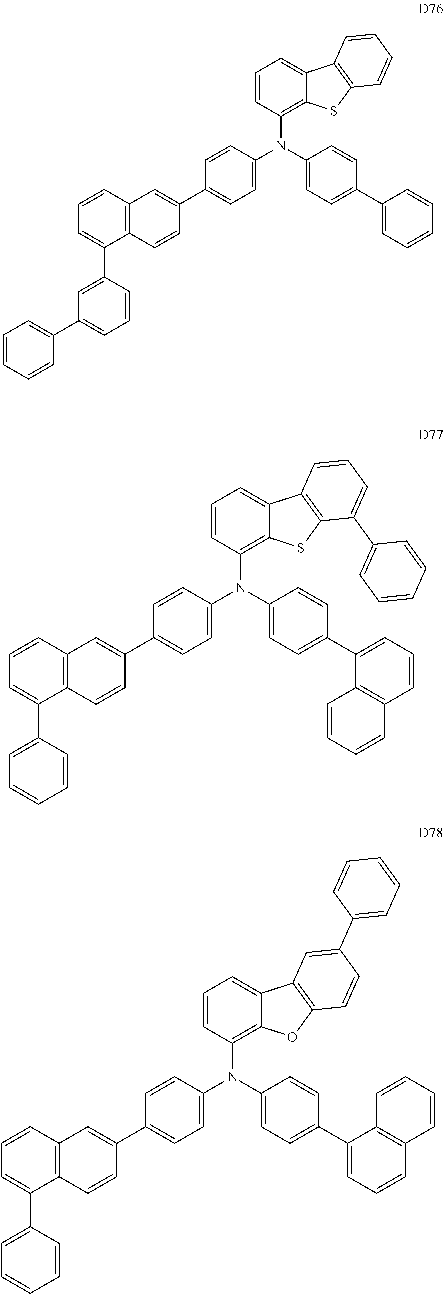 [SMILES: c1ccc(-c2ccc(N(c3ccc(-c4ccc5c(-c6cccc(-c7ccccc7)c6)cccc5c4)cc3)c3cccc4c3sc3ccccc34)cc2)cc1 ; c1ccc(-c2cccc3cc(-c4ccc(N(c5ccc(-c6cccc7ccccc67)cc5)c5cccc6c5sc5c(-c7ccccc7)cccc56)cc4)ccc23)cc1 ; c1ccc(-c2ccc3oc4c(N(c5ccc(-c6ccc7c(-c8ccccc8)cccc7c6)cc5)c5ccc(-c6cccc7ccccc67)cc5)cccc4c3c2)cc1] 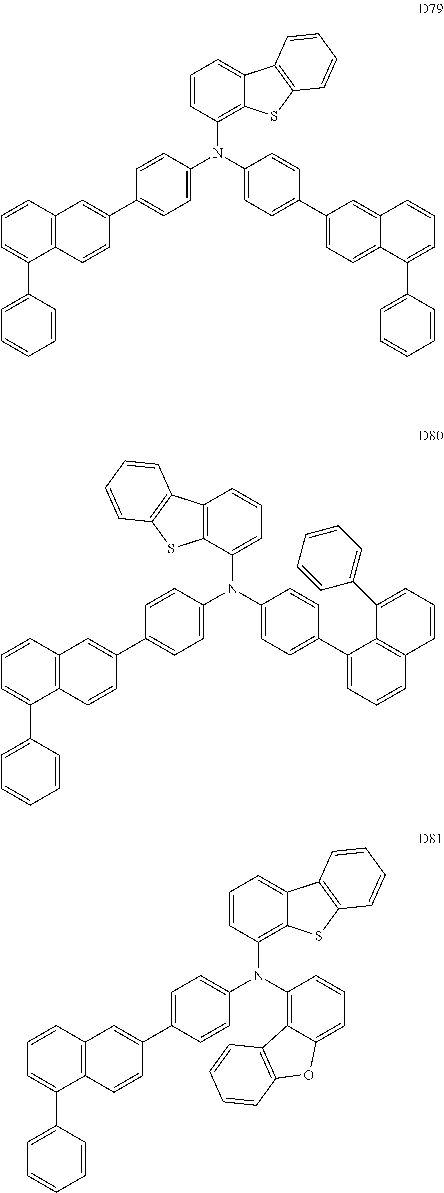 [SMILES: c1ccc(-c2cccc3cc(-c4ccc(N(c5ccc(-c6ccc7c(-c8ccccc8)cccc7c6)cc5)c5cccc6c5sc5ccccc56)cc4)ccc23)cc1 ; c1ccc(-c2cccc3cc(-c4ccc(N(c5ccc(-c6cccc7cccc(-c8ccccc8)c67)cc5)c5cccc6c5sc5ccccc56)cc4)ccc23)cc1 ; c1ccc(-c2cccc3cc(-c4ccc(N(c5cccc6c5sc5ccccc56)c5cccc6oc7ccccc7c56)cc4)ccc23)cc1] 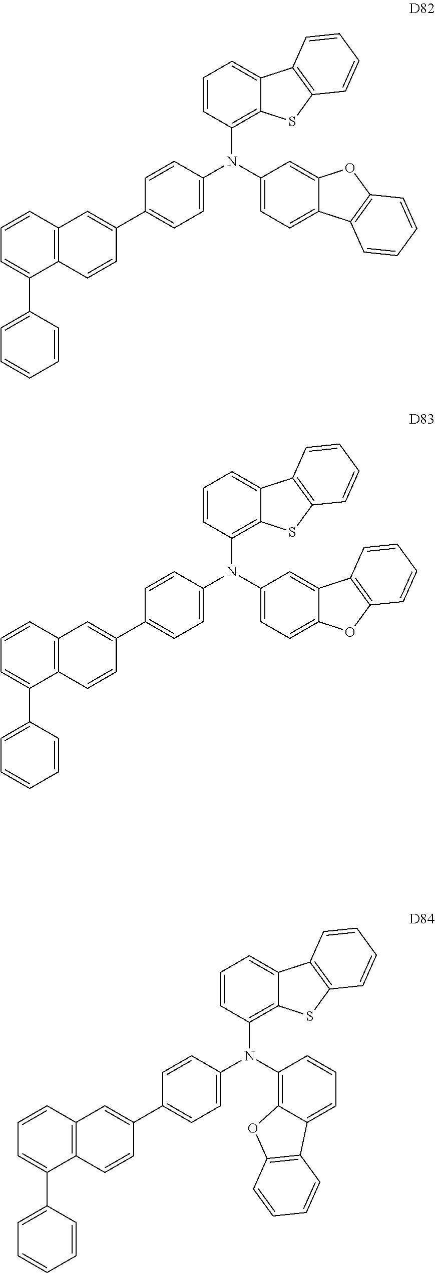 [SMILES: c1ccc(-c2cccc3cc(-c4ccc(N(c5ccc6c(c5)oc5ccccc56)c5cccc6c5sc5ccccc56)cc4)ccc23)cc1 ; c1ccc(-c2cccc3cc(-c4ccc(N(c5ccc6oc7ccccc7c6c5)c5cccc6c5sc5ccccc56)cc4)ccc23)cc1 ; c1ccc(-c2cccc3cc(-c4ccc(N(c5cccc6c5oc5ccccc56)c5cccc6c5sc5ccccc56)cc4)ccc23)cc1] 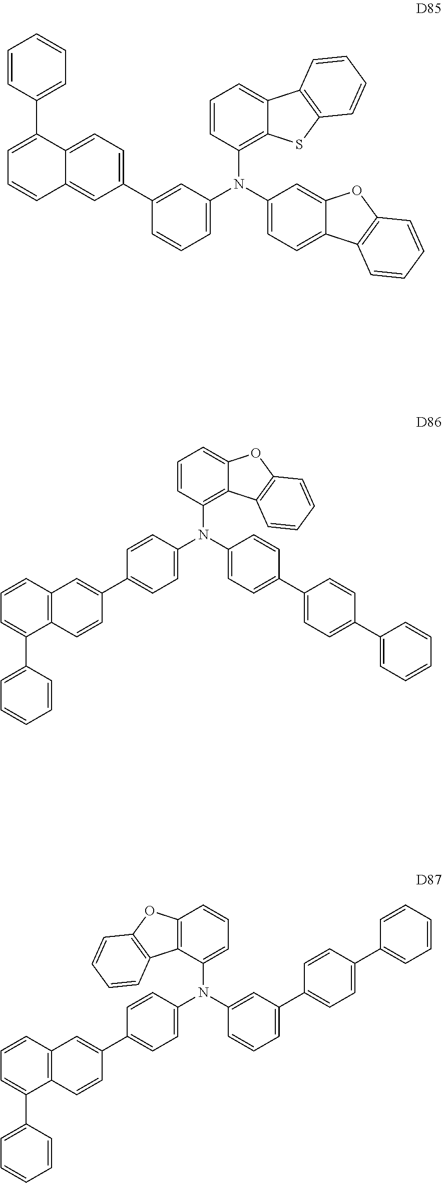 [SMILES: c1ccc(-c2cccc3cc(-c4cccc(N(c5ccc6c(c5)oc5ccccc56)c5cccc6c5sc5ccccc56)c4)ccc23)cc1 ; c1ccc(-c2ccc(-c3ccc(N(c4ccc(-c5ccc6c(-c7ccccc7)cccc6c5)cc4)c4cccc5oc6ccccc6c45)cc3)cc2)cc1 ; c1ccc(-c2ccc(-c3cccc(N(c4ccc(-c5ccc6c(-c7ccccc7)cccc6c5)cc4)c4cccc5oc6ccccc6c45)c3)cc2)cc1] 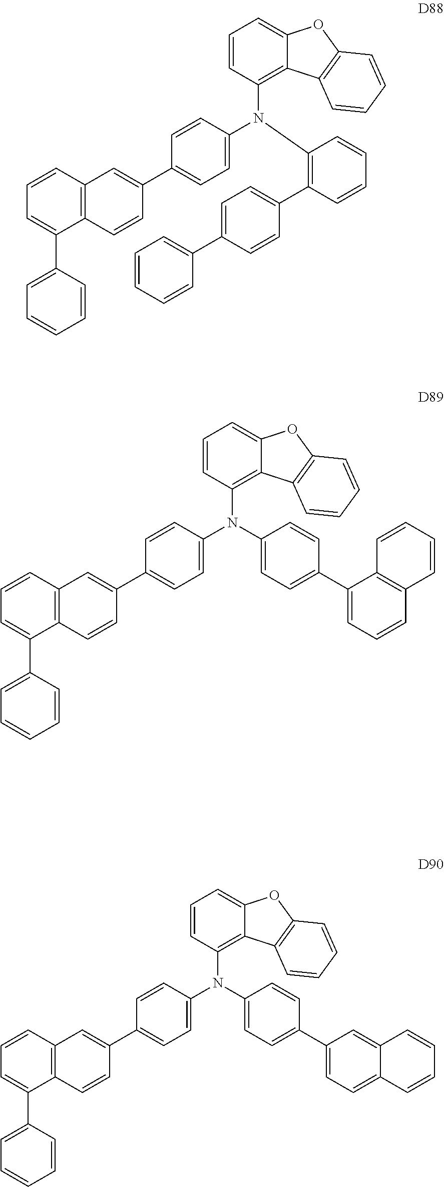 [SMILES: c1ccc(-c2ccc(-c3ccccc3N(c3ccc(-c4ccc5c(-c6ccccc6)cccc5c4)cc3)c3cccc4oc5ccccc5c34)cc2)cc1 ; c1ccc(-c2cccc3cc(-c4ccc(N(c5ccc(-c6cccc7ccccc67)cc5)c5cccc6oc7ccccc7c56)cc4)ccc23)cc1 ; c1ccc(-c2cccc3cc(-c4ccc(N(c5ccc(-c6ccc7ccccc7c6)cc5)c5cccc6oc7ccccc7c56)cc4)ccc23)cc1] 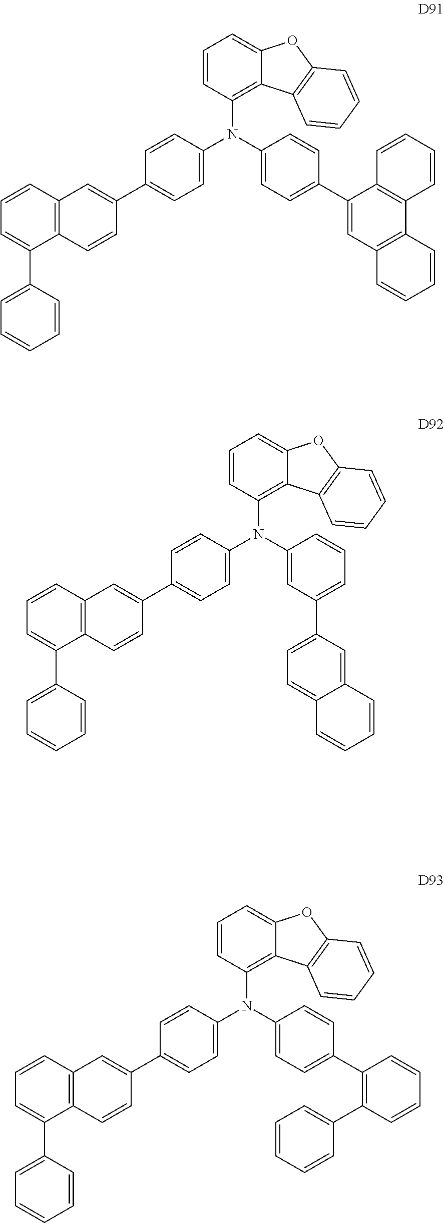 [SMILES: c1ccc(-c2cccc3cc(-c4ccc(N(c5ccc(-c6cc7ccccc7c7ccccc67)cc5)c5cccc6oc7ccccc7c56)cc4)ccc23)cc1 ; c1ccc(-c2cccc3cc(-c4ccc(N(c5cccc(-c6ccc7ccccc7c6)c5)c5cccc6oc7ccccc7c56)cc4)ccc23)cc1 ; c1ccc(-c2ccccc2-c2ccc(N(c3ccc(-c4ccc5c(-c6ccccc6)cccc5c4)cc3)c3cccc4oc5ccccc5c34)cc2)cc1] 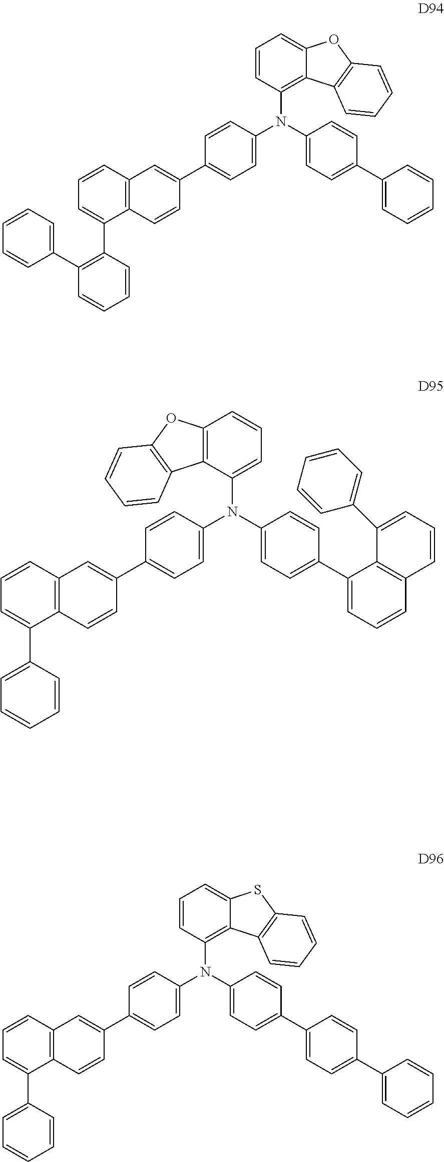 [SMILES: c1ccc(-c2ccc(N(c3ccc(-c4ccc5c(-c6ccccc6-c6ccccc6)cccc5c4)cc3)c3cccc4oc5ccccc5c34)cc2)cc1 ; c1ccc(-c2cccc3cc(-c4ccc(N(c5ccc(-c6cccc7cccc(-c8ccccc8)c67)cc5)c5cccc6oc7ccccc7c56)cc4)ccc23)cc1 ; c1ccc(-c2ccc(-c3ccc(N(c4ccc(-c5ccc6c(-c7ccccc7)cccc6c5)cc4)c4cccc5sc6ccccc6c45)cc3)cc2)cc1] 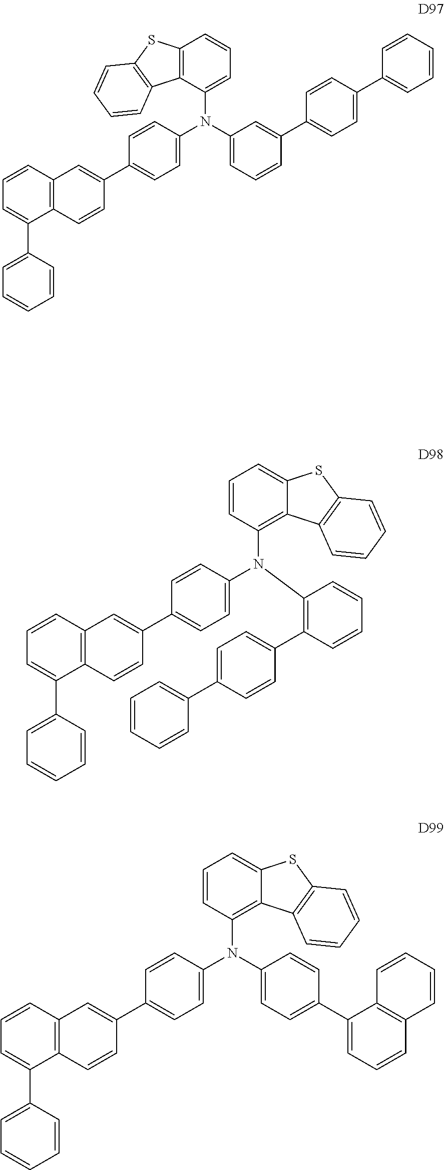 [SMILES: c1ccc(-c2ccc(-c3cccc(N(c4ccc(-c5ccc6c(-c7ccccc7)cccc6c5)cc4)c4cccc5sc6ccccc6c45)c3)cc2)cc1 ; c1ccc(-c2ccc(-c3ccccc3N(c3ccc(-c4ccc5c(-c6ccccc6)cccc5c4)cc3)c3cccc4sc5ccccc5c34)cc2)cc1 ; c1ccc(-c2cccc3cc(-c4ccc(N(c5ccc(-c6cccc7ccccc67)cc5)c5cccc6sc7ccccc7c56)cc4)ccc23)cc1] 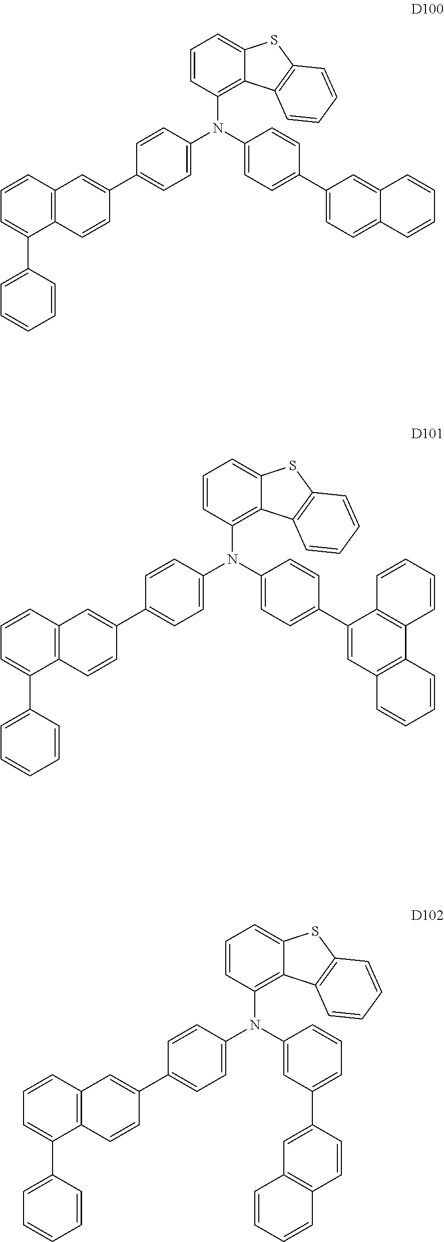 [SMILES: c1ccc(-c2cccc3cc(-c4ccc(N(c5ccc(-c6ccc7ccccc7c6)cc5)c5cccc6sc7ccccc7c56)cc4)ccc23)cc1 ; c1ccc(-c2cccc3cc(-c4ccc(N(c5ccc(-c6cc7ccccc7c7ccccc67)cc5)c5cccc6sc7ccccc7c56)cc4)ccc23)cc1 ; c1ccc(-c2cccc3cc(-c4ccc(N(c5cccc(-c6ccc7ccccc7c6)c5)c5cccc6sc7ccccc7c56)cc4)ccc23)cc1] 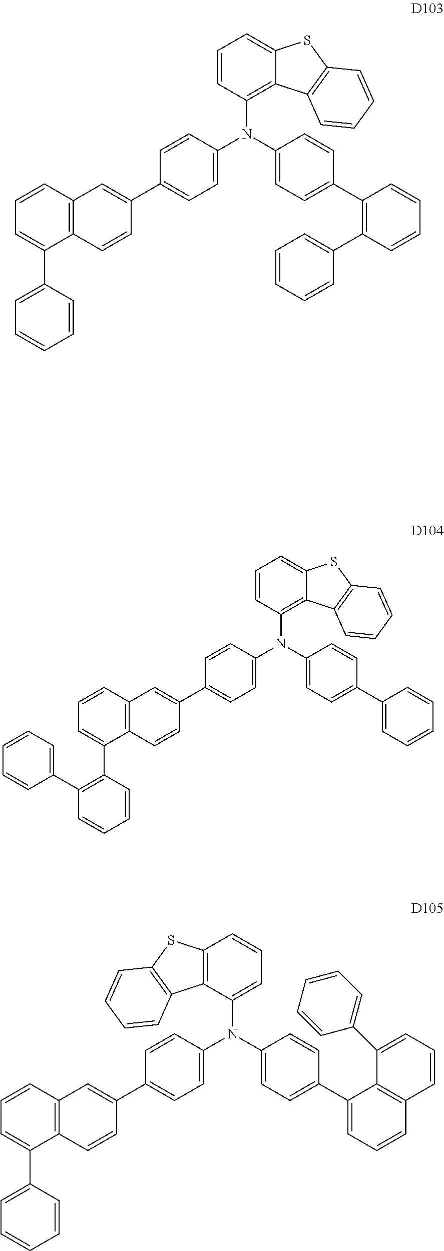 [SMILES: c1ccc(-c2ccccc2-c2ccc(N(c3ccc(-c4ccc5c(-c6ccccc6)cccc5c4)cc3)c3cccc4sc5ccccc5c34)cc2)cc1 ; c1ccc(-c2ccc(N(c3ccc(-c4ccc5c(-c6ccccc6-c6ccccc6)cccc5c4)cc3)c3cccc4sc5ccccc5c34)cc2)cc1 ; c1ccc(-c2cccc3cc(-c4ccc(N(c5ccc(-c6cccc7cccc(-c8ccccc8)c67)cc5)c5cccc6sc7ccccc7c56)cc4)ccc23)cc1] 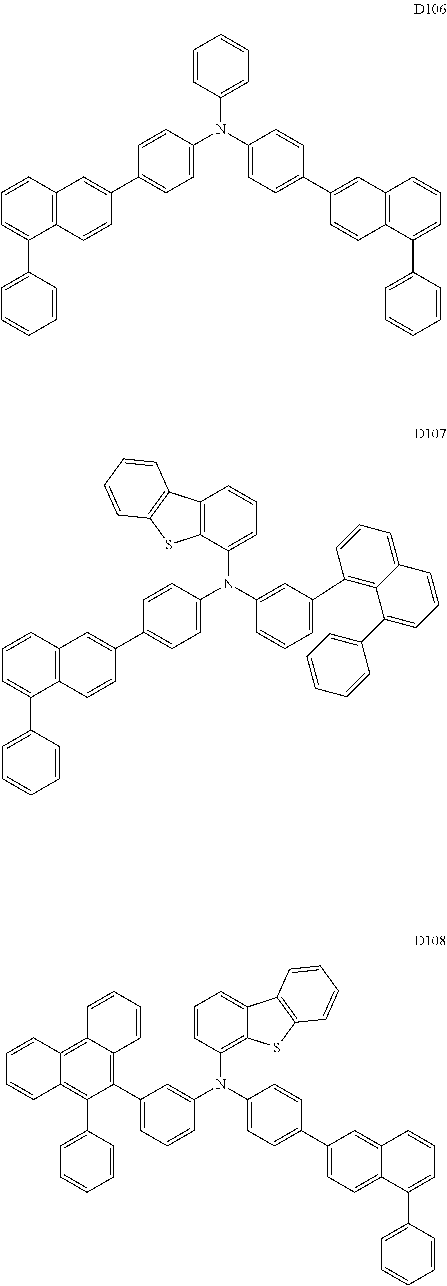 [SMILES: c1ccc(-c2cccc3cc(-c4ccc(N(c5ccccc5)c5ccc(-c6ccc7c(-c8ccccc8)cccc7c6)cc5)cc4)ccc23)cc1 ; c1ccc(-c2cccc3cc(-c4ccc(N(c5cccc(-c6cccc7cccc(-c8ccccc8)c67)c5)c5cccc6c5sc5ccccc56)cc4)ccc23)cc1 ; c1ccc(-c2cccc3cc(-c4ccc(N(c5cccc(-c6c(-c7ccccc7)c7ccccc7c7ccccc67)c5)c5cccc6c5sc5ccccc56)cc4)ccc23)cc1] 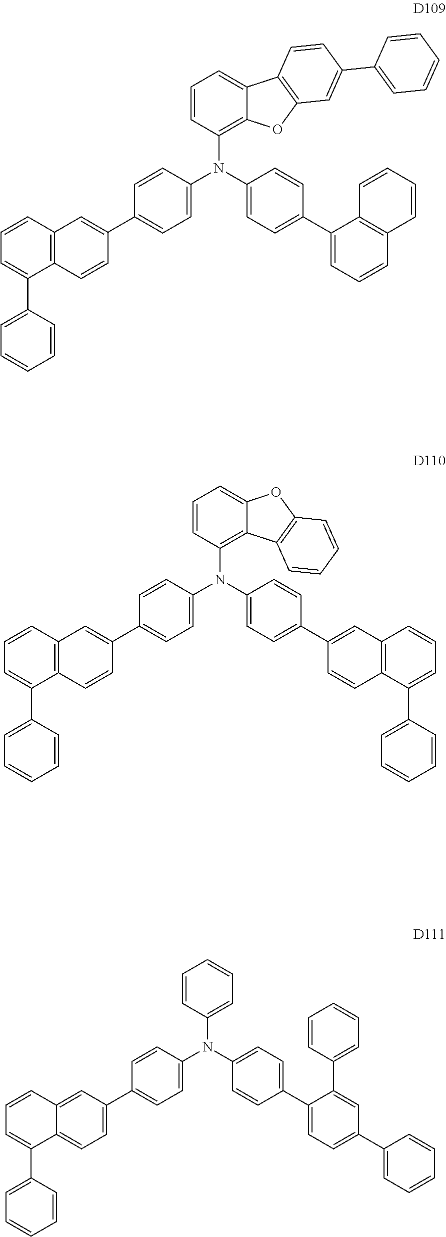 [SMILES: c1ccc(-c2ccc3c(c2)oc2c(N(c4ccc(-c5ccc6c(-c7ccccc7)cccc6c5)cc4)c4ccc(-c5cccc6ccccc56)cc4)cccc23)cc1 ; c1ccc(-c2cccc3cc(-c4ccc(N(c5ccc(-c6ccc7c(-c8ccccc8)cccc7c6)cc5)c5cccc6oc7ccccc7c56)cc4)ccc23)cc1 ; c1ccc(-c2ccc(-c3ccc(N(c4ccccc4)c4ccc(-c5ccc6c(-c7ccccc7)cccc6c5)cc4)cc3)c(-c3ccccc3)c2)cc1] 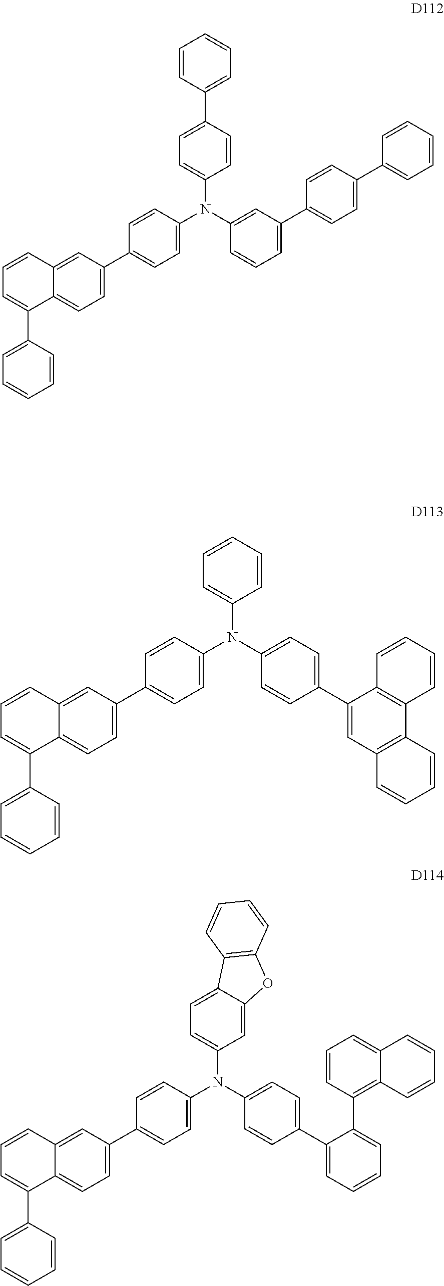 [SMILES: c1ccc(-c2ccc(-c3cccc(N(c4ccc(-c5ccccc5)cc4)c4ccc(-c5ccc6c(-c7ccccc7)cccc6c5)cc4)c3)cc2)cc1 ; c1ccc(-c2cccc3cc(-c4ccc(N(c5ccccc5)c5ccc(-c6cc7ccccc7c7ccccc67)cc5)cc4)ccc23)cc1 ; c1ccc(-c2cccc3cc(-c4ccc(N(c5ccc(-c6ccccc6-c6cccc7ccccc67)cc5)c5ccc6c(c5)oc5ccccc56)cc4)ccc23)cc1] 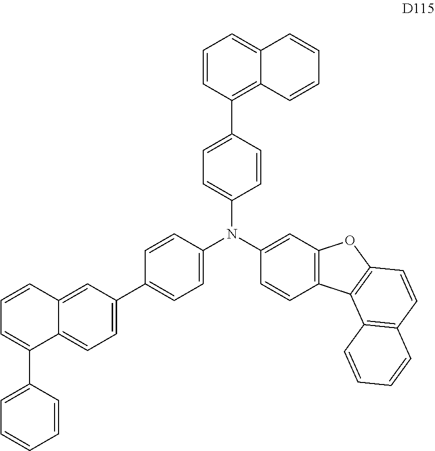 [SMILES: c1ccc(-c2cccc3cc(-c4ccc(N(c5ccc(-c6cccc7ccccc67)cc5)c5ccc6c(c5)oc5ccc7ccccc7c56)cc4)ccc23)cc1] 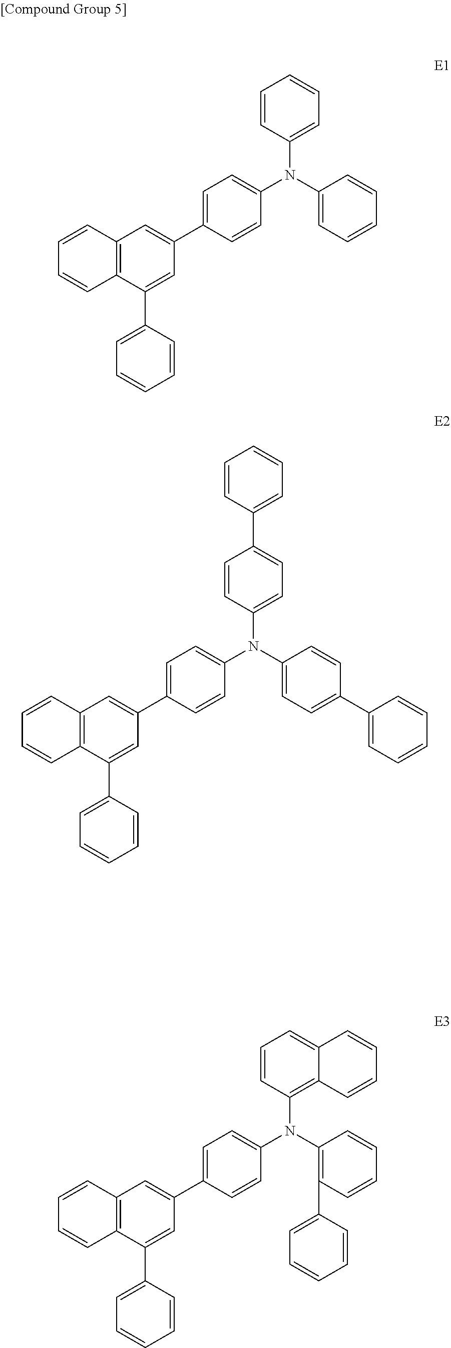 [SMILES: c1ccc(-c2cc(-c3ccc(N(c4ccccc4)c4ccccc4)cc3)cc3ccccc23)cc1 ; c1ccc(-c2ccc(N(c3ccc(-c4ccccc4)cc3)c3ccc(-c4cc(-c5ccccc5)c5ccccc5c4)cc3)cc2)cc1 ; c1ccc(-c2ccccc2N(c2ccc(-c3cc(-c4ccccc4)c4ccccc4c3)cc2)c2cccc3ccccc23)cc1] 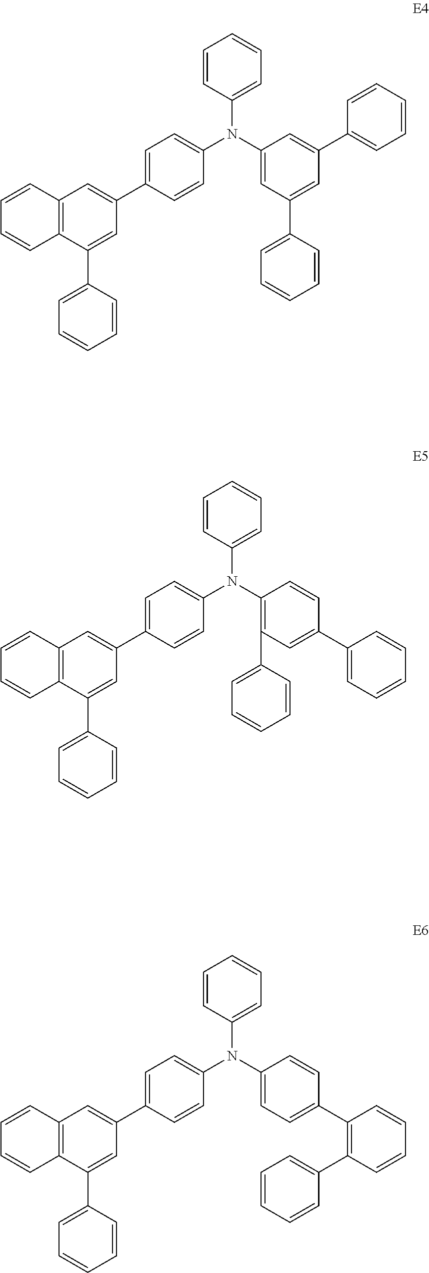 [SMILES: c1ccc(-c2cc(-c3ccccc3)cc(N(c3ccccc3)c3ccc(-c4cc(-c5ccccc5)c5ccccc5c4)cc3)c2)cc1 ; c1ccc(-c2ccc(N(c3ccccc3)c3ccc(-c4cc(-c5ccccc5)c5ccccc5c4)cc3)c(-c3ccccc3)c2)cc1 ; c1ccc(-c2ccccc2-c2ccc(N(c3ccccc3)c3ccc(-c4cc(-c5ccccc5)c5ccccc5c4)cc3)cc2)cc1] 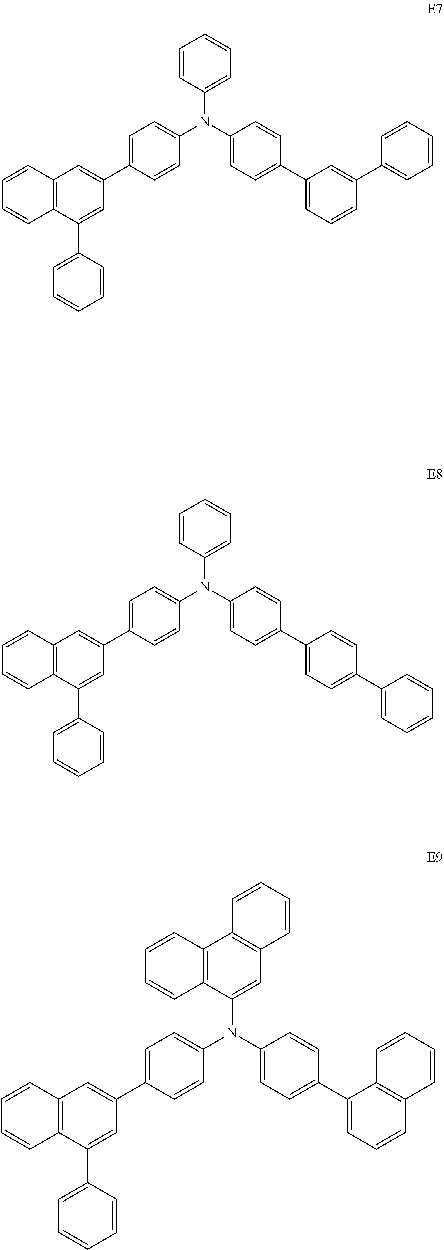 [SMILES: c1ccc(-c2cccc(-c3ccc(N(c4ccccc4)c4ccc(-c5cc(-c6ccccc6)c6ccccc6c5)cc4)cc3)c2)cc1 ; c1ccc(-c2ccc(-c3ccc(N(c4ccccc4)c4ccc(-c5cc(-c6ccccc6)c6ccccc6c5)cc4)cc3)cc2)cc1 ; c1ccc(-c2cc(-c3ccc(N(c4ccc(-c5cccc6ccccc56)cc4)c4cc5ccccc5c5ccccc45)cc3)cc3ccccc23)cc1] 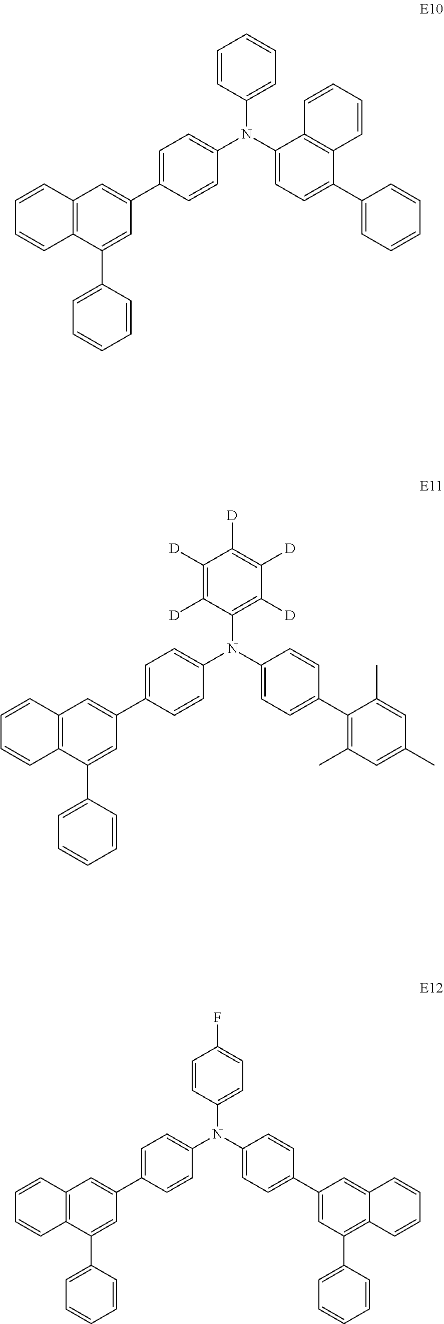 [SMILES: c1ccc(-c2cc(-c3ccc(N(c4ccccc4)c4ccc(-c5ccccc5)c5ccccc45)cc3)cc3ccccc23)cc1 ; [2H]c1c([2H])c([2H])c(N(c2ccc(-c3cc(-c4ccccc4)c4ccccc4c3)cc2)c2ccc(-c3c(C)cc(C)cc3C)cc2)c([2H])c1[2H] ; Fc1ccc(N(c2ccc(-c3cc(-c4ccccc4)c4ccccc4c3)cc2)c2ccc(-c3cc(-c4ccccc4)c4ccccc4c3)cc2)cc1] 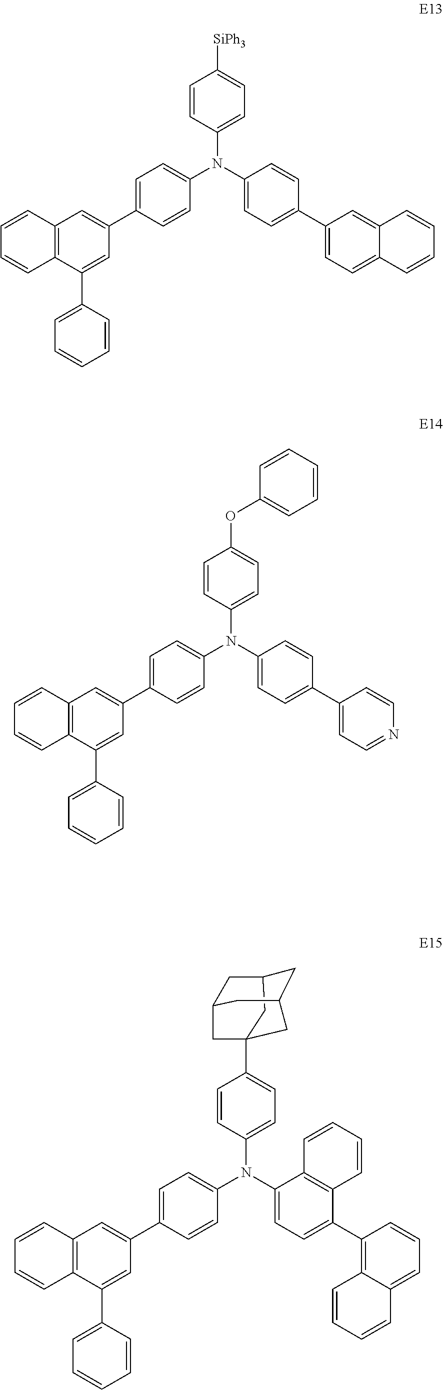 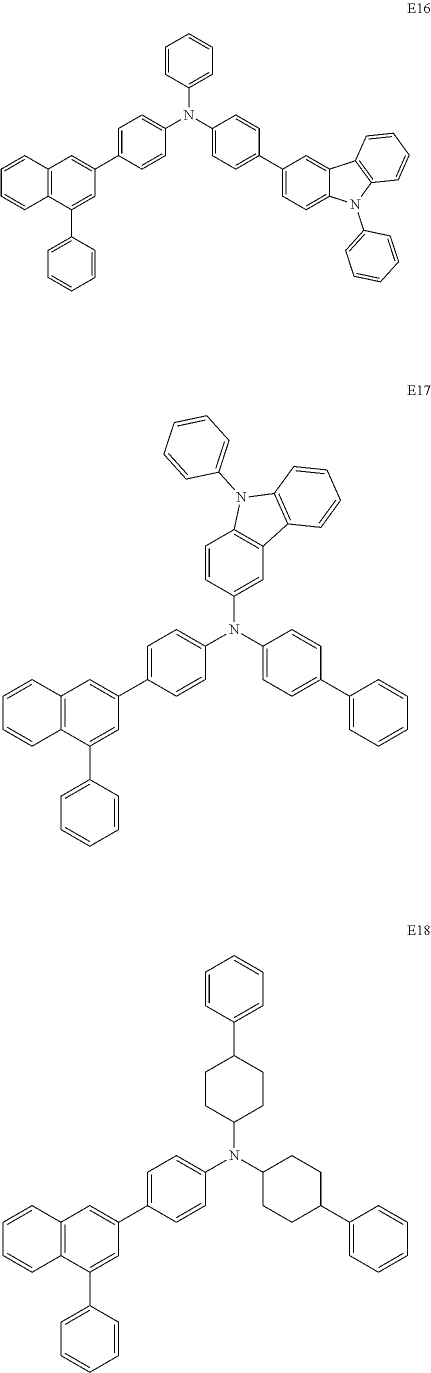 [SMILES: c1ccc(-c2cc(-c3ccc(N(c4ccccc4)c4ccc(-c5ccc6c(c5)c5ccccc5n6-c5ccccc5)cc4)cc3)cc3ccccc23)cc1 ; c1ccc(-c2ccc(N(c3ccc(-c4cc(-c5ccccc5)c5ccccc5c4)cc3)c3ccc4c(c3)c3ccccc3n4-c3ccccc3)cc2)cc1 ; c1ccc(-c2cc(-c3ccc(N(C4CCC(c5ccccc5)CC4)C4CCC(c5ccccc5)CC4)cc3)cc3ccccc23)cc1] 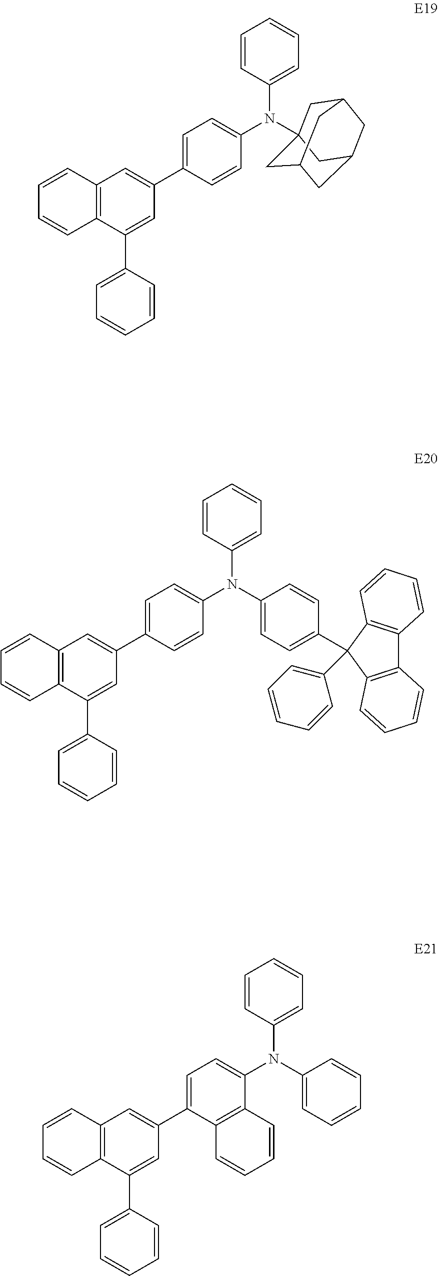 [SMILES: c1ccc(-c2cc(-c3ccc(N(c4ccccc4)C45CC6CC(CC(C6)C4)C5)cc3)cc3ccccc23)cc1 ; c1ccc(-c2cc(-c3ccc(N(c4ccccc4)c4ccc(C5(c6ccccc6)c6ccccc6-c6ccccc65)cc4)cc3)cc3ccccc23)cc1 ; c1ccc(-c2cc(-c3ccc(N(c4ccccc4)c4ccccc4)c4ccccc34)cc3ccccc23)cc1] 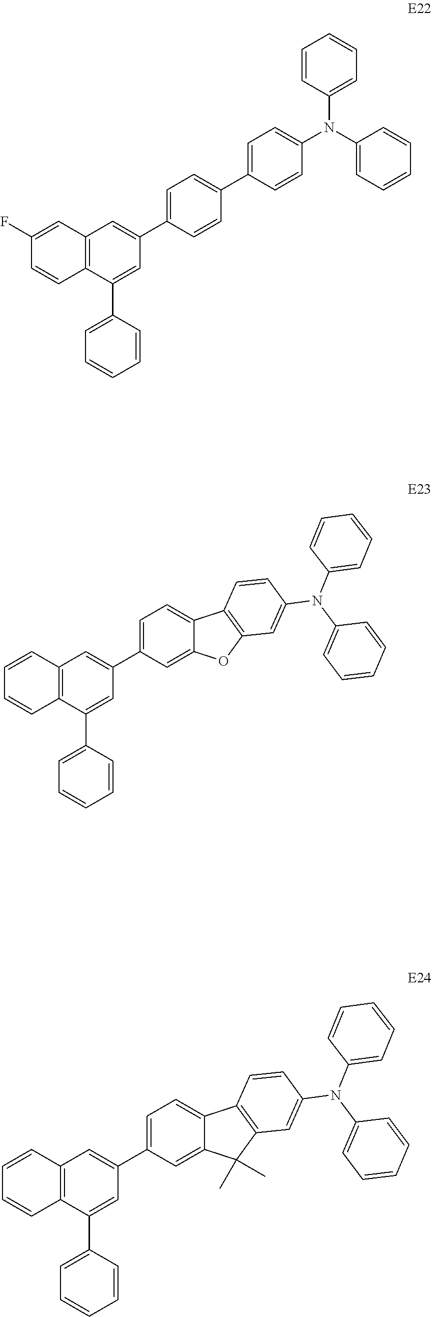 [SMILES: Fc1ccc2c(-c3ccccc3)cc(-c3ccc(-c4ccc(N(c5ccccc5)c5ccccc5)cc4)cc3)cc2c1 ; c1ccc(-c2cc(-c3ccc4c(c3)oc3cc(N(c5ccccc5)c5ccccc5)ccc34)cc3ccccc23)cc1 ; CC1(C)c2cc(-c3cc(-c4ccccc4)c4ccccc4c3)ccc2-c2ccc(N(c3ccccc3)c3ccccc3)cc21] 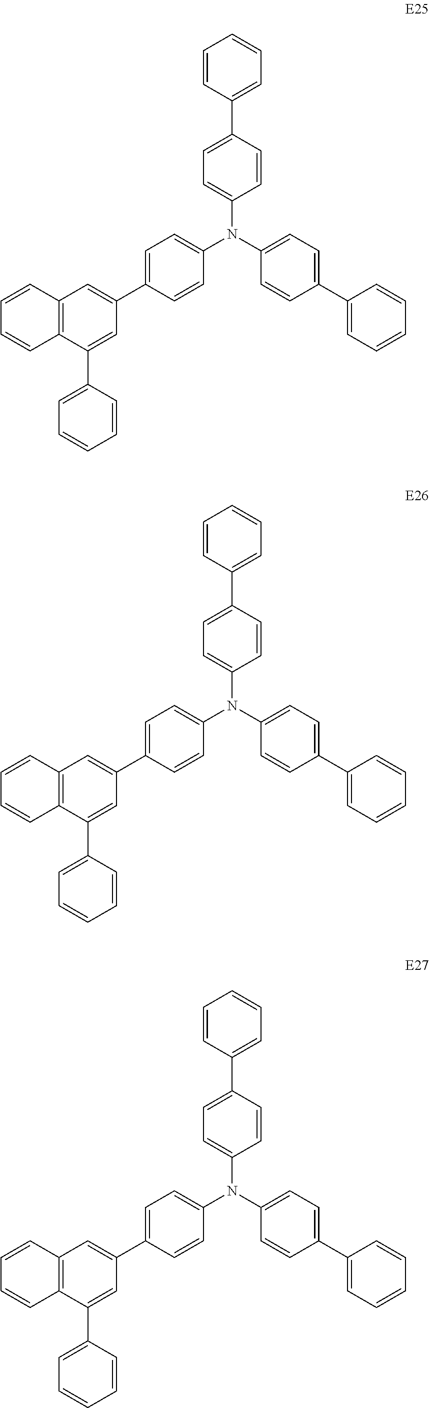 [SMILES: c1ccc(-c2ccc(N(c3ccc(-c4ccccc4)cc3)c3ccc(-c4cc(-c5ccccc5)c5ccccc5c4)cc3)cc2)cc1 ; c1ccc(-c2ccc(N(c3ccc(-c4ccccc4)cc3)c3ccc(-c4cc(-c5ccccc5)c5ccccc5c4)cc3)cc2)cc1 ; c1ccc(-c2ccc(N(c3ccc(-c4ccccc4)cc3)c3ccc(-c4cc(-c5ccccc5)c5ccccc5c4)cc3)cc2)cc1] 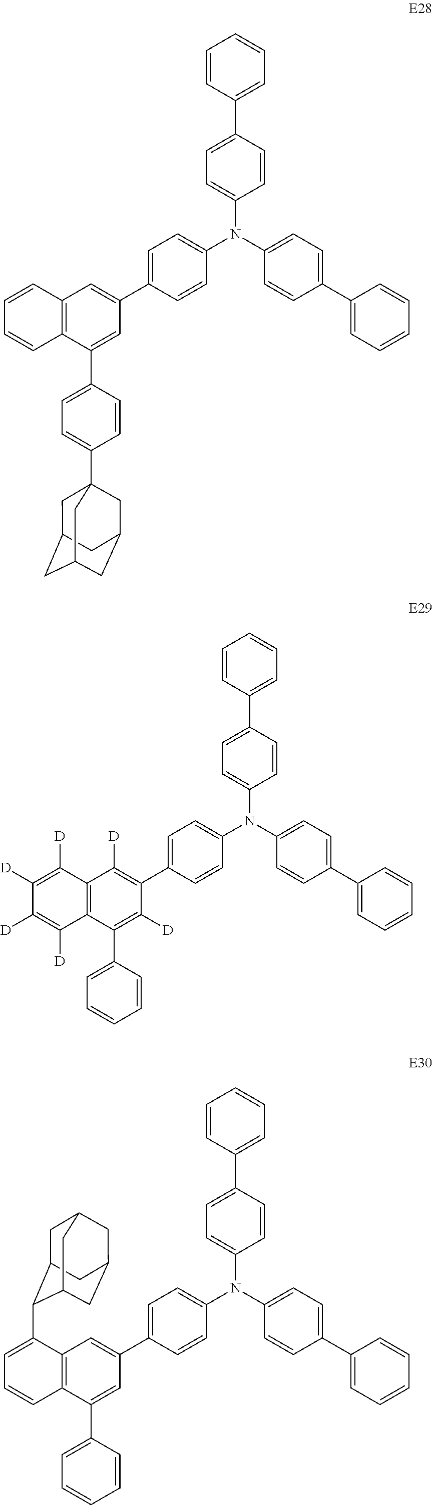 [SMILES: c1ccc(-c2ccc(N(c3ccc(-c4ccccc4)cc3)c3ccc(-c4cc(-c5ccc(C67CC8CC(CC(C8)C6)C7)cc5)c5ccccc5c4)cc3)cc2)cc1 ; [2H]c1c([2H])c([2H])c2c(-c3ccccc3)c([2H])c(-c3ccc(N(c4ccc(-c5ccccc5)cc4)c4ccc(-c5ccccc5)cc4)cc3)c([2H])c2c1[2H] ; c1ccc(-c2ccc(N(c3ccc(-c4ccccc4)cc3)c3ccc(-c4cc(-c5ccccc5)c5cccc(C6C7CC8CC(C7)CC6C8)c5c4)cc3)cc2)cc1] 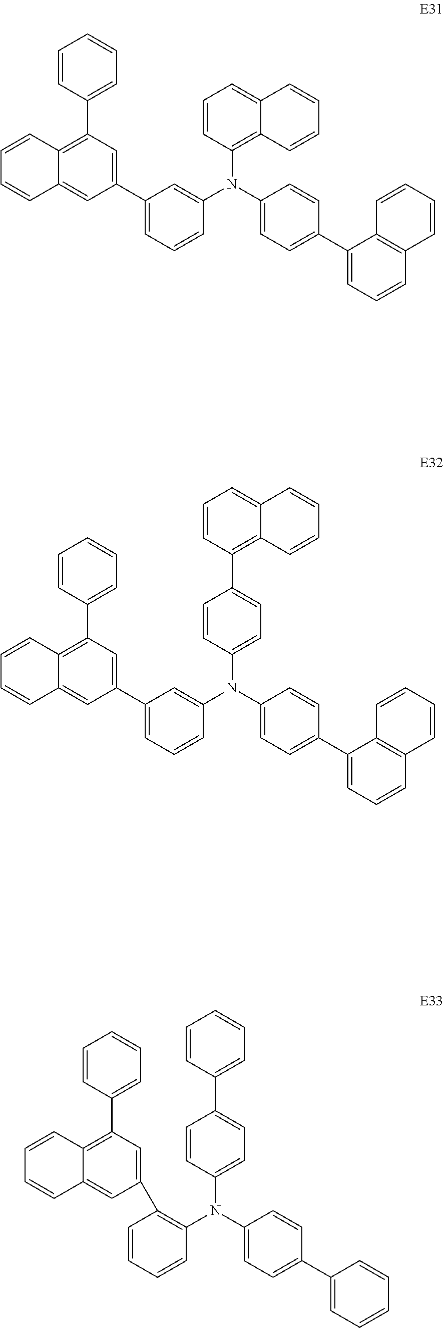 [SMILES: c1ccc(-c2cc(-c3cccc(N(c4ccc(-c5cccc6ccccc56)cc4)c4cccc5ccccc45)c3)cc3ccccc23)cc1 ; c1ccc(-c2cc(-c3cccc(N(c4ccc(-c5cccc6ccccc56)cc4)c4ccc(-c5cccc6ccccc56)cc4)c3)cc3ccccc23)cc1 ; c1ccc(-c2ccc(N(c3ccc(-c4ccccc4)cc3)c3ccccc3-c3cc(-c4ccccc4)c4ccccc4c3)cc2)cc1] 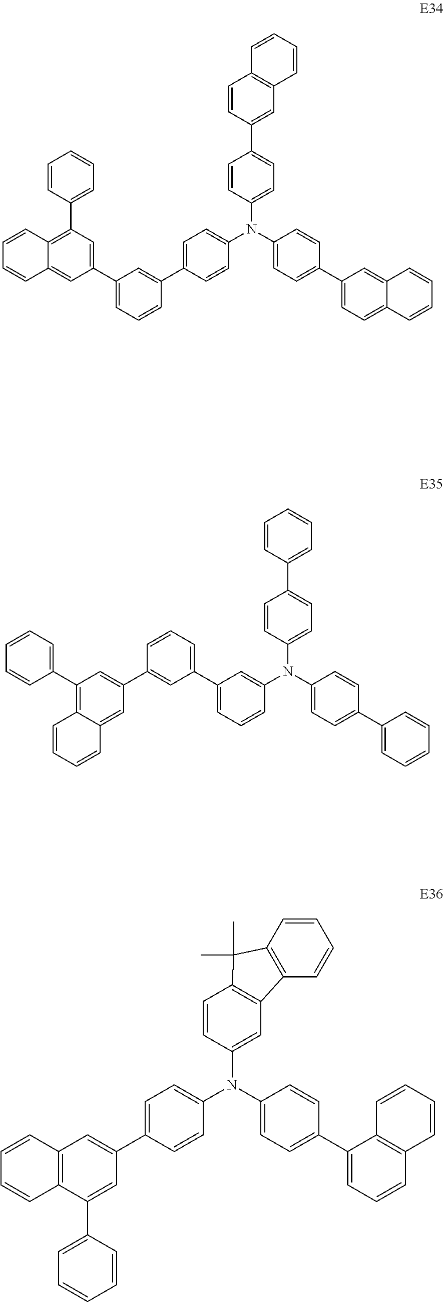 [SMILES: c1ccc(-c2cc(-c3cccc(-c4ccc(N(c5ccc(-c6ccc7ccccc7c6)cc5)c5ccc(-c6ccc7ccccc7c6)cc5)cc4)c3)cc3ccccc23)cc1 ; c1ccc(-c2ccc(N(c3ccc(-c4ccccc4)cc3)c3cccc(-c4cccc(-c5cc(-c6ccccc6)c6ccccc6c5)c4)c3)cc2)cc1 ; CC1(C)c2ccccc2-c2cc(N(c3ccc(-c4cc(-c5ccccc5)c5ccccc5c4)cc3)c3ccc(-c4cccc5ccccc45)cc3)ccc21] 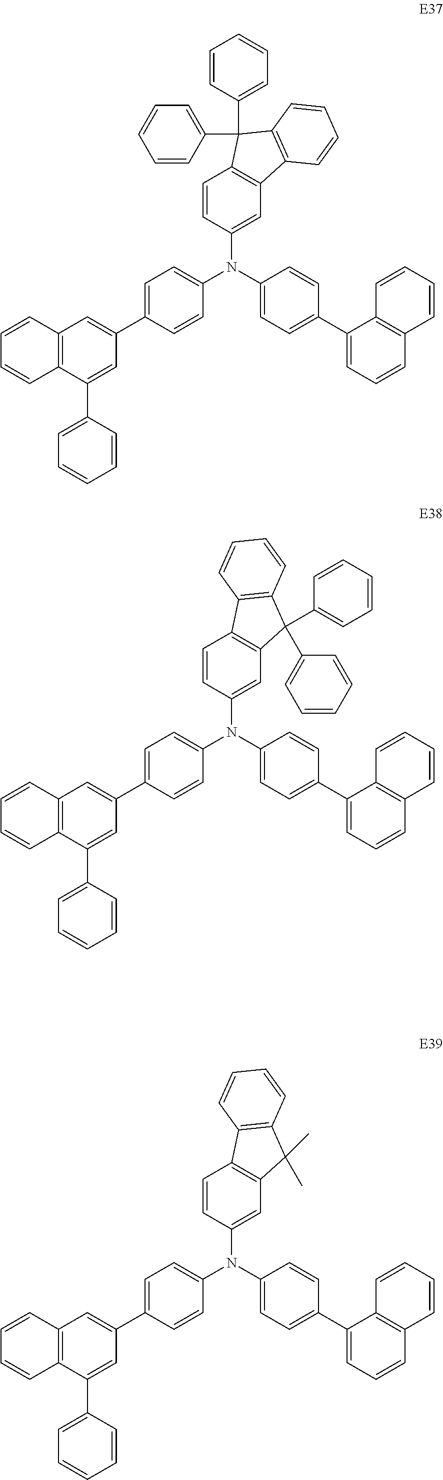 [SMILES: c1ccc(-c2cc(-c3ccc(N(c4ccc(-c5cccc6ccccc56)cc4)c4ccc5c(c4)-c4ccccc4C5(c4ccccc4)c4ccccc4)cc3)cc3ccccc23)cc1 ; c1ccc(-c2cc(-c3ccc(N(c4ccc(-c5cccc6ccccc56)cc4)c4ccc5c(c4)C(c4ccccc4)(c4ccccc4)c4ccccc4-5)cc3)cc3ccccc23)cc1 ; CC1(C)c2ccccc2-c2ccc(N(c3ccc(-c4cc(-c5ccccc5)c5ccccc5c4)cc3)c3ccc(-c4cccc5ccccc45)cc3)cc21] 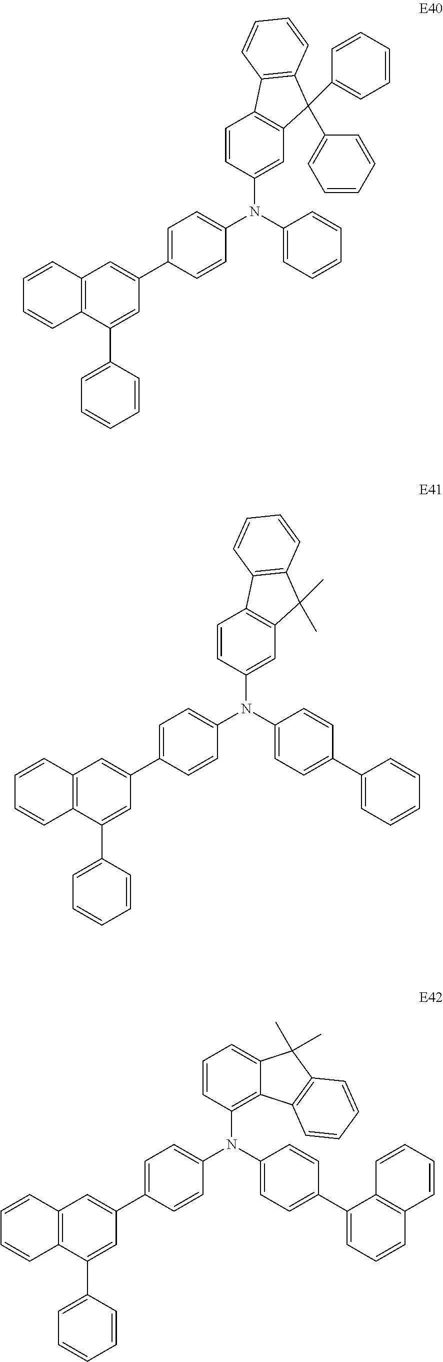 [SMILES: c1ccc(-c2cc(-c3ccc(N(c4ccccc4)c4ccc5c(c4)C(c4ccccc4)(c4ccccc4)c4ccccc4-5)cc3)cc3ccccc23)cc1 ; CC1(C)c2ccccc2-c2ccc(N(c3ccc(-c4ccccc4)cc3)c3ccc(-c4cc(-c5ccccc5)c5ccccc5c4)cc3)cc21 ; CC1(C)c2ccccc2-c2c(N(c3ccc(-c4cc(-c5ccccc5)c5ccccc5c4)cc3)c3ccc(-c4cccc5ccccc45)cc3)cccc21] 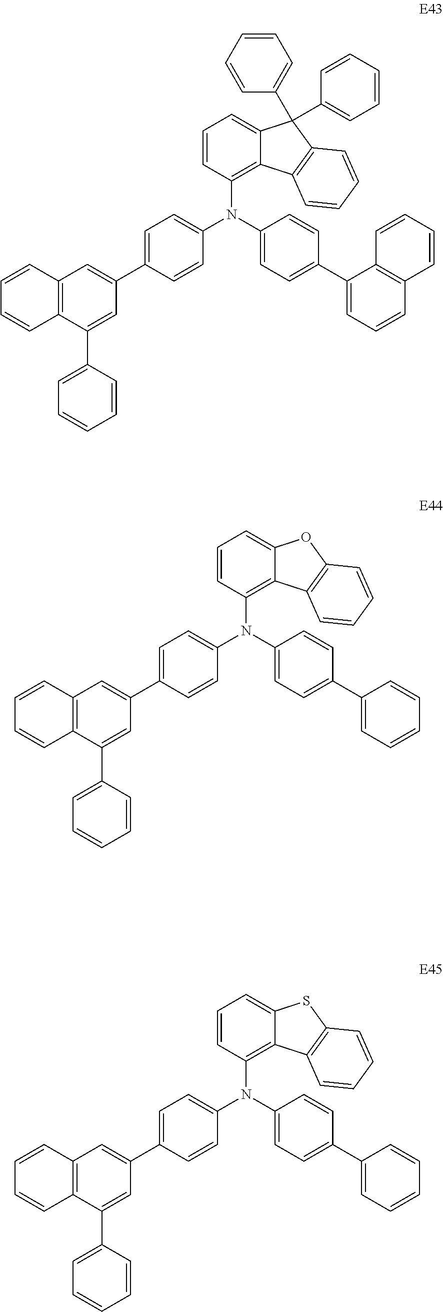 [SMILES: c1ccc(-c2cc(-c3ccc(N(c4ccc(-c5cccc6ccccc56)cc4)c4cccc5c4-c4ccccc4C5(c4ccccc4)c4ccccc4)cc3)cc3ccccc23)cc1 ; c1ccc(-c2ccc(N(c3ccc(-c4cc(-c5ccccc5)c5ccccc5c4)cc3)c3cccc4oc5ccccc5c34)cc2)cc1 ; c1ccc(-c2ccc(N(c3ccc(-c4cc(-c5ccccc5)c5ccccc5c4)cc3)c3cccc4sc5ccccc5c34)cc2)cc1] 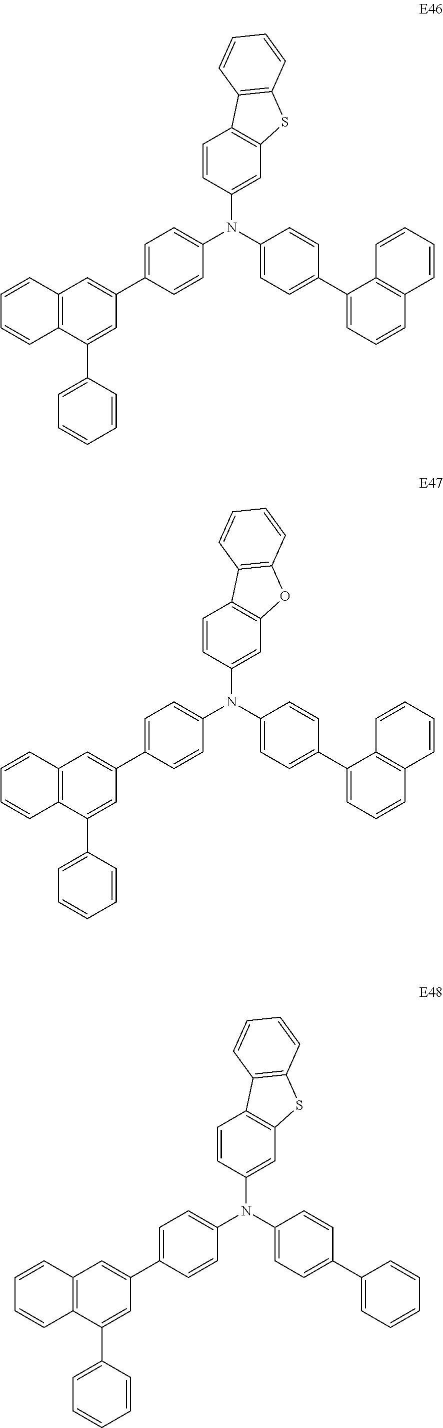 [SMILES: c1ccc(-c2cc(-c3ccc(N(c4ccc(-c5cccc6ccccc56)cc4)c4ccc5c(c4)sc4ccccc45)cc3)cc3ccccc23)cc1 ; c1ccc(-c2cc(-c3ccc(N(c4ccc(-c5cccc6ccccc56)cc4)c4ccc5c(c4)oc4ccccc45)cc3)cc3ccccc23)cc1 ; c1ccc(-c2ccc(N(c3ccc(-c4cc(-c5ccccc5)c5ccccc5c4)cc3)c3ccc4c(c3)sc3ccccc34)cc2)cc1] 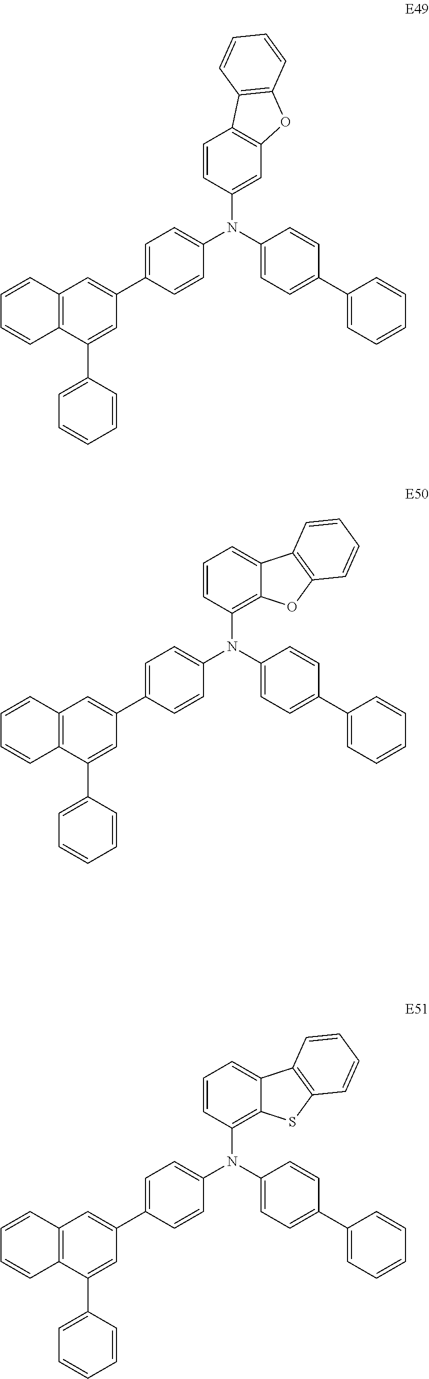 [SMILES: c1ccc(-c2ccc(N(c3ccc(-c4cc(-c5ccccc5)c5ccccc5c4)cc3)c3ccc4c(c3)oc3ccccc34)cc2)cc1 ; c1ccc(-c2ccc(N(c3ccc(-c4cc(-c5ccccc5)c5ccccc5c4)cc3)c3cccc4c3oc3ccccc34)cc2)cc1 ; c1ccc(-c2ccc(N(c3ccc(-c4cc(-c5ccccc5)c5ccccc5c4)cc3)c3cccc4c3sc3ccccc34)cc2)cc1] 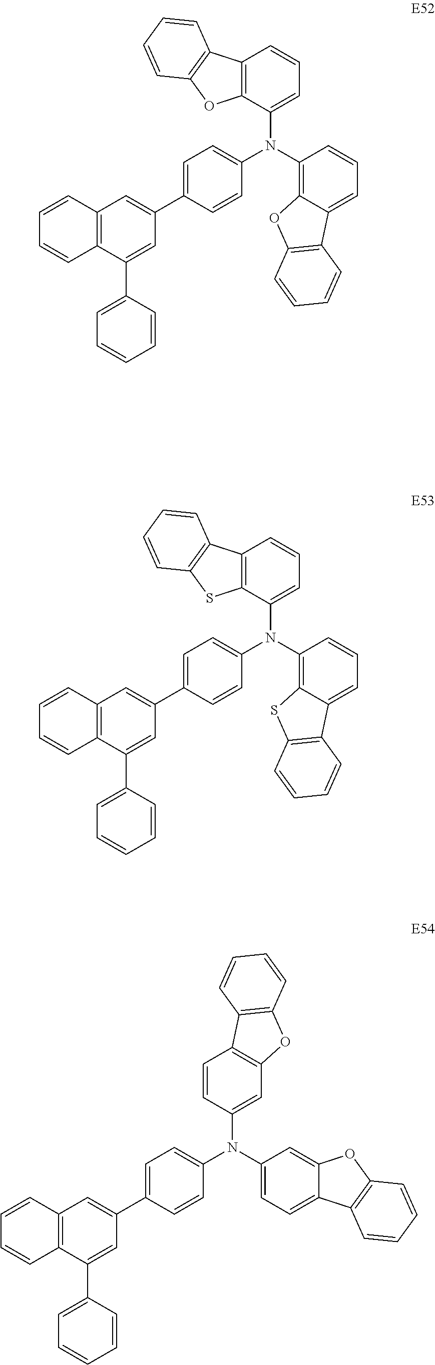 [SMILES: c1ccc(-c2cc(-c3ccc(N(c4cccc5c4oc4ccccc45)c4cccc5c4oc4ccccc45)cc3)cc3ccccc23)cc1 ; c1ccc(-c2cc(-c3ccc(N(c4cccc5c4sc4ccccc45)c4cccc5c4sc4ccccc45)cc3)cc3ccccc23)cc1 ; c1ccc(-c2cc(-c3ccc(N(c4ccc5c(c4)oc4ccccc45)c4ccc5c(c4)oc4ccccc45)cc3)cc3ccccc23)cc1] 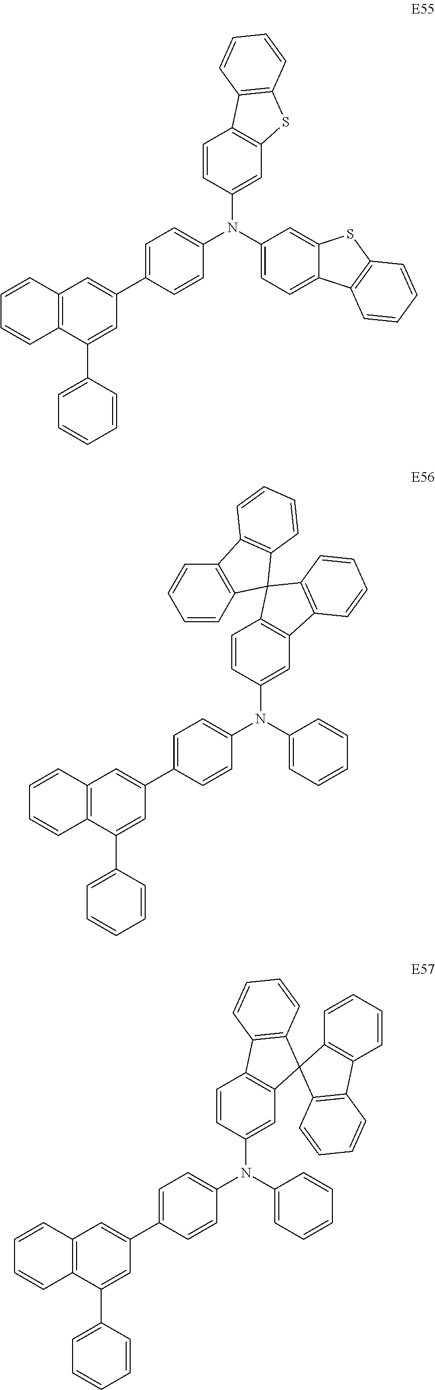 [SMILES: c1ccc(-c2cc(-c3ccc(N(c4ccc5c(c4)sc4ccccc45)c4ccc5c(c4)sc4ccccc45)cc3)cc3ccccc23)cc1 ; c1ccc(-c2cc(-c3ccc(N(c4ccccc4)c4ccc5c(c4)-c4ccccc4C54c5ccccc5-c5ccccc54)cc3)cc3ccccc23)cc1 ; c1ccc(-c2cc(-c3ccc(N(c4ccccc4)c4ccc5c(c4)C4(c6ccccc6-c6ccccc64)c4ccccc4-5)cc3)cc3ccccc23)cc1] 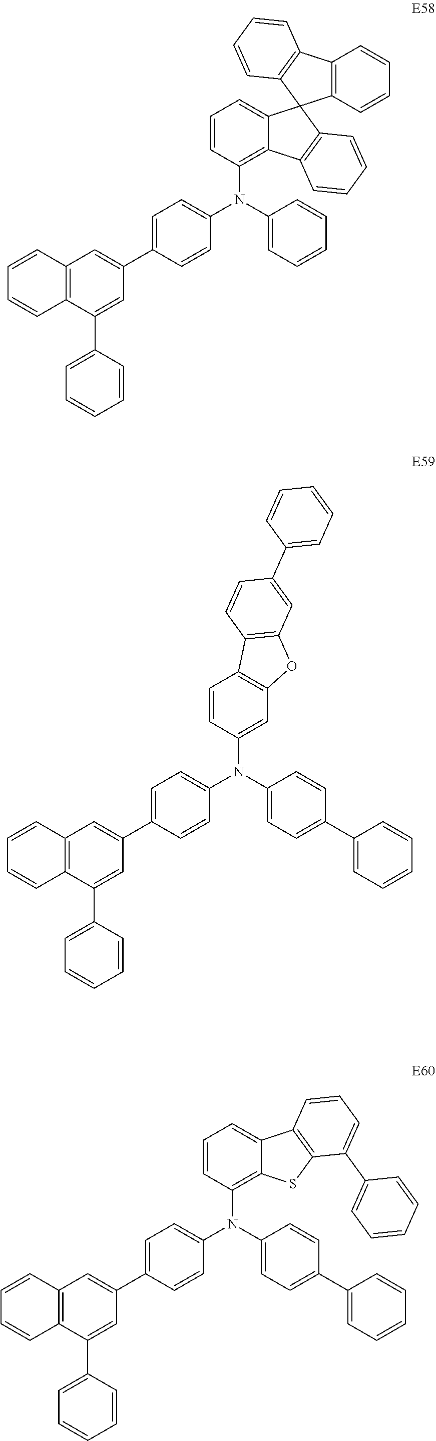 [SMILES: c1ccc(-c2cc(-c3ccc(N(c4ccccc4)c4cccc5c4-c4ccccc4C54c5ccccc5-c5ccccc54)cc3)cc3ccccc23)cc1 ; c1ccc(-c2ccc(N(c3ccc(-c4cc(-c5ccccc5)c5ccccc5c4)cc3)c3ccc4c(c3)oc3cc(-c5ccccc5)ccc34)cc2)cc1 ; c1ccc(-c2ccc(N(c3ccc(-c4cc(-c5ccccc5)c5ccccc5c4)cc3)c3cccc4c3sc3c(-c5ccccc5)cccc34)cc2)cc1] 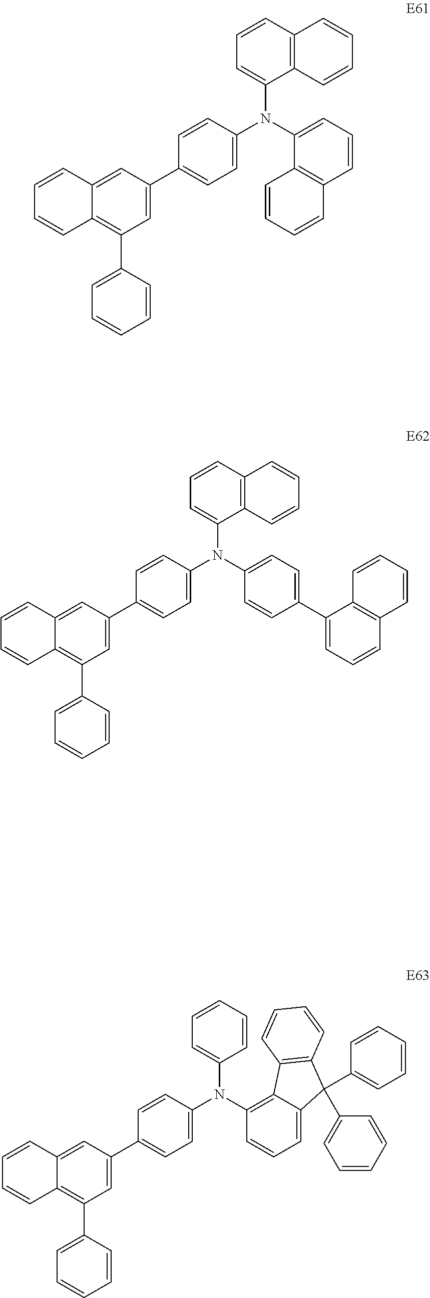 [SMILES: c1ccc(-c2cc(-c3ccc(N(c4cccc5ccccc45)c4cccc5ccccc45)cc3)cc3ccccc23)cc1 ; c1ccc(-c2cc(-c3ccc(N(c4ccc(-c5cccc6ccccc56)cc4)c4cccc5ccccc45)cc3)cc3ccccc23)cc1 ; c1ccc(-c2cc(-c3ccc(N(c4ccccc4)c4cccc5c4-c4ccccc4C5(c4ccccc4)c4ccccc4)cc3)cc3ccccc23)cc1] 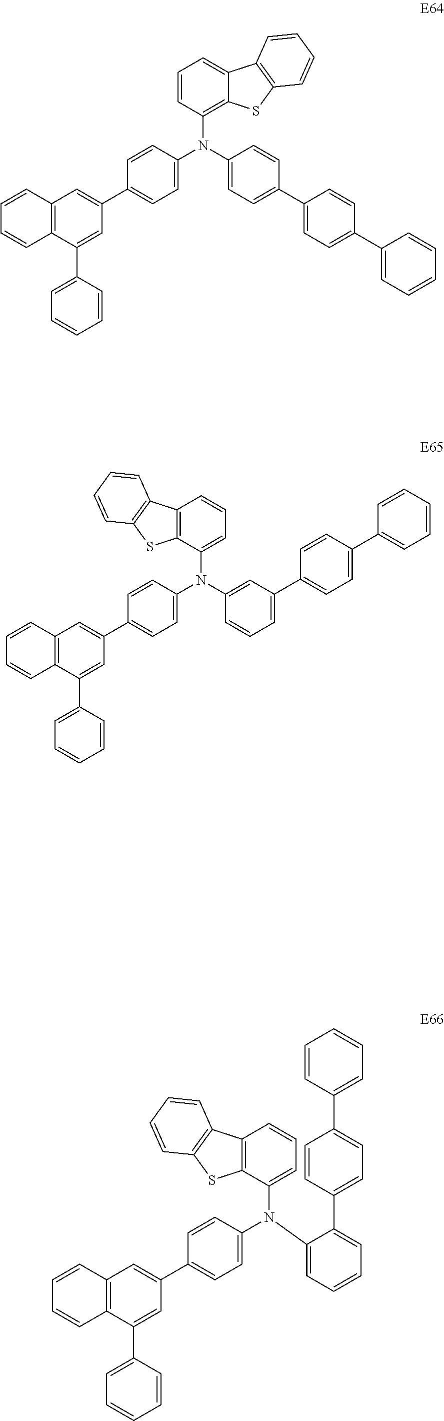 [SMILES: c1ccc(-c2ccc(-c3ccc(N(c4ccc(-c5cc(-c6ccccc6)c6ccccc6c5)cc4)c4cccc5c4sc4ccccc45)cc3)cc2)cc1 ; c1ccc(-c2ccc(-c3cccc(N(c4ccc(-c5cc(-c6ccccc6)c6ccccc6c5)cc4)c4cccc5c4sc4ccccc45)c3)cc2)cc1 ; c1ccc(-c2ccc(-c3ccccc3N(c3ccc(-c4cc(-c5ccccc5)c5ccccc5c4)cc3)c3cccc4c3sc3ccccc34)cc2)cc1] 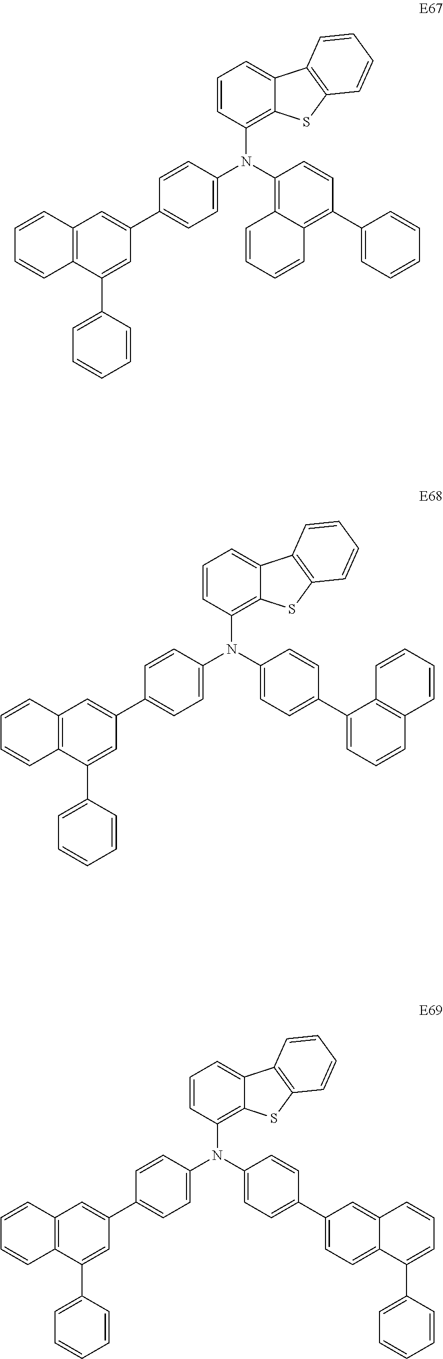 [SMILES: c1ccc(-c2cc(-c3ccc(N(c4ccc(-c5ccccc5)c5ccccc45)c4cccc5c4sc4ccccc45)cc3)cc3ccccc23)cc1 ; c1ccc(-c2cc(-c3ccc(N(c4ccc(-c5cccc6ccccc56)cc4)c4cccc5c4sc4ccccc45)cc3)cc3ccccc23)cc1 ; c1ccc(-c2cccc3cc(-c4ccc(N(c5ccc(-c6cc(-c7ccccc7)c7ccccc7c6)cc5)c5cccc6c5sc5ccccc56)cc4)ccc23)cc1] 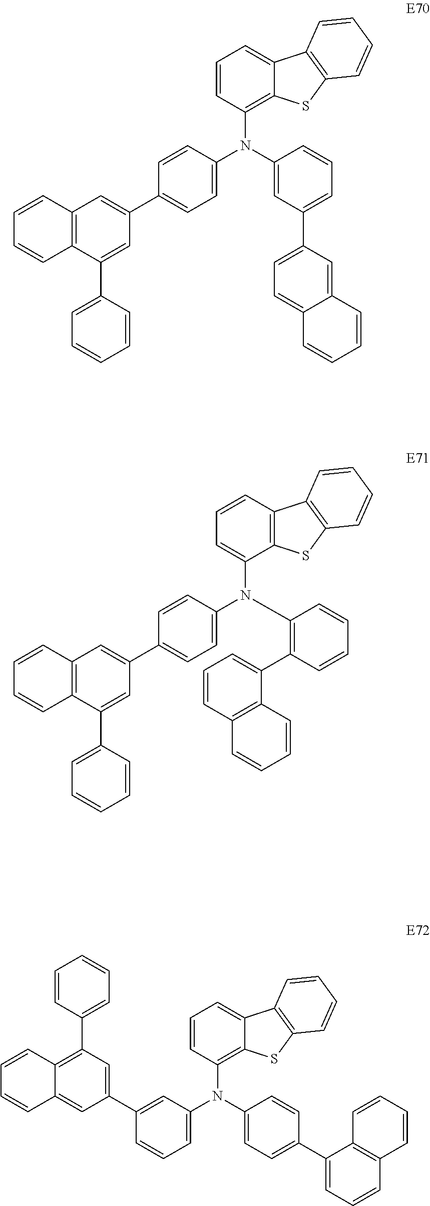 [SMILES: c1ccc(-c2cc(-c3ccc(N(c4cccc(-c5ccc6ccccc6c5)c4)c4cccc5c4sc4ccccc45)cc3)cc3ccccc23)cc1 ; c1ccc(-c2cc(-c3ccc(N(c4ccccc4-c4cccc5ccccc45)c4cccc5c4sc4ccccc45)cc3)cc3ccccc23)cc1 ; c1ccc(-c2cc(-c3cccc(N(c4ccc(-c5cccc6ccccc56)cc4)c4cccc5c4sc4ccccc45)c3)cc3ccccc23)cc1] 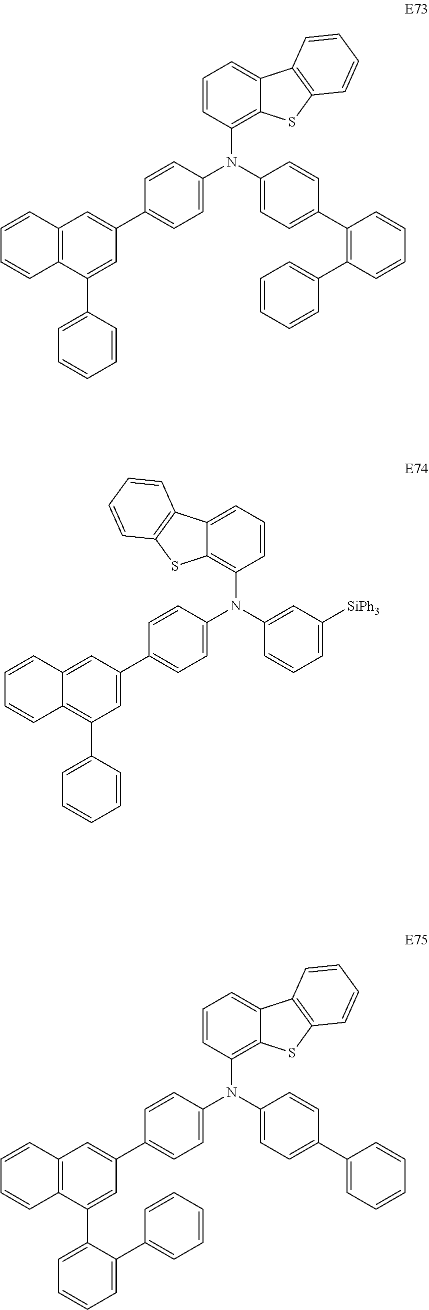 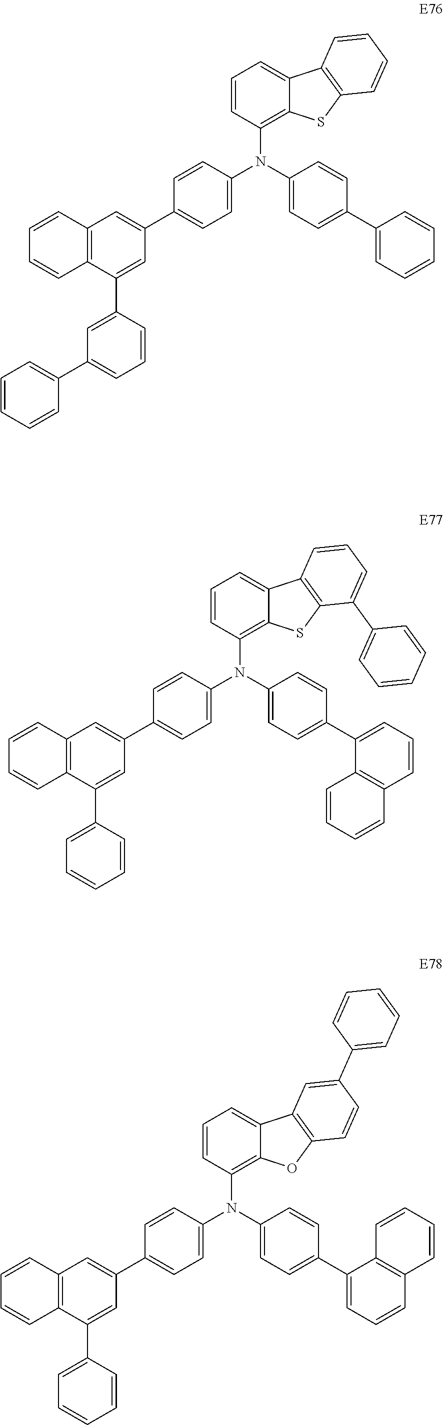 [SMILES: c1ccc(-c2ccc(N(c3ccc(-c4cc(-c5cccc(-c6ccccc6)c5)c5ccccc5c4)cc3)c3cccc4c3sc3ccccc34)cc2)cc1 ; c1ccc(-c2cc(-c3ccc(N(c4ccc(-c5cccc6ccccc56)cc4)c4cccc5c4sc4c(-c6ccccc6)cccc45)cc3)cc3ccccc23)cc1 ; c1ccc(-c2ccc3oc4c(N(c5ccc(-c6cc(-c7ccccc7)c7ccccc7c6)cc5)c5ccc(-c6cccc7ccccc67)cc5)cccc4c3c2)cc1] 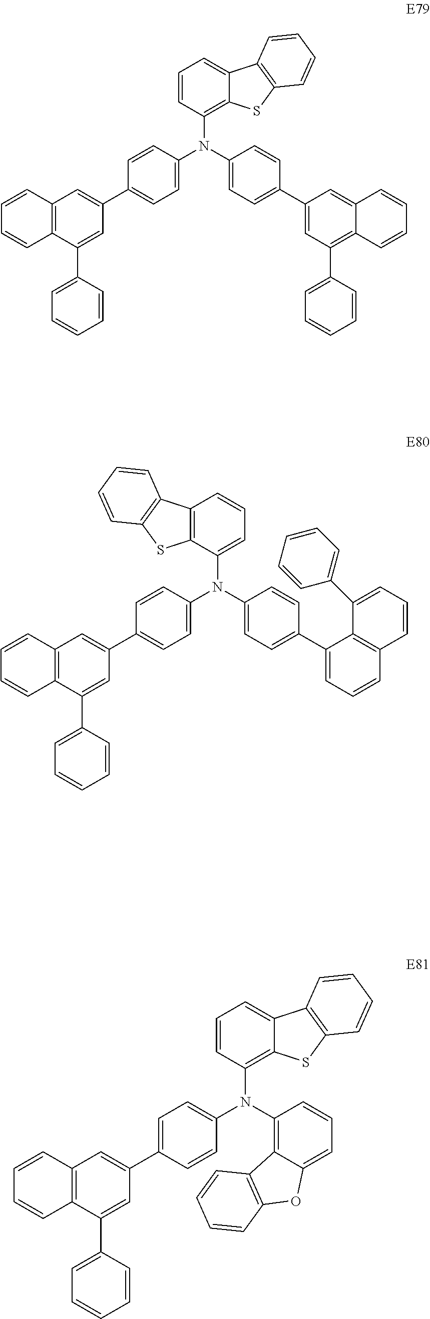 [SMILES: c1ccc(-c2cc(-c3ccc(N(c4ccc(-c5cc(-c6ccccc6)c6ccccc6c5)cc4)c4cccc5c4sc4ccccc45)cc3)cc3ccccc23)cc1 ; c1ccc(-c2cc(-c3ccc(N(c4ccc(-c5cccc6cccc(-c7ccccc7)c56)cc4)c4cccc5c4sc4ccccc45)cc3)cc3ccccc23)cc1 ; c1ccc(-c2cc(-c3ccc(N(c4cccc5c4sc4ccccc45)c4cccc5oc6ccccc6c45)cc3)cc3ccccc23)cc1] 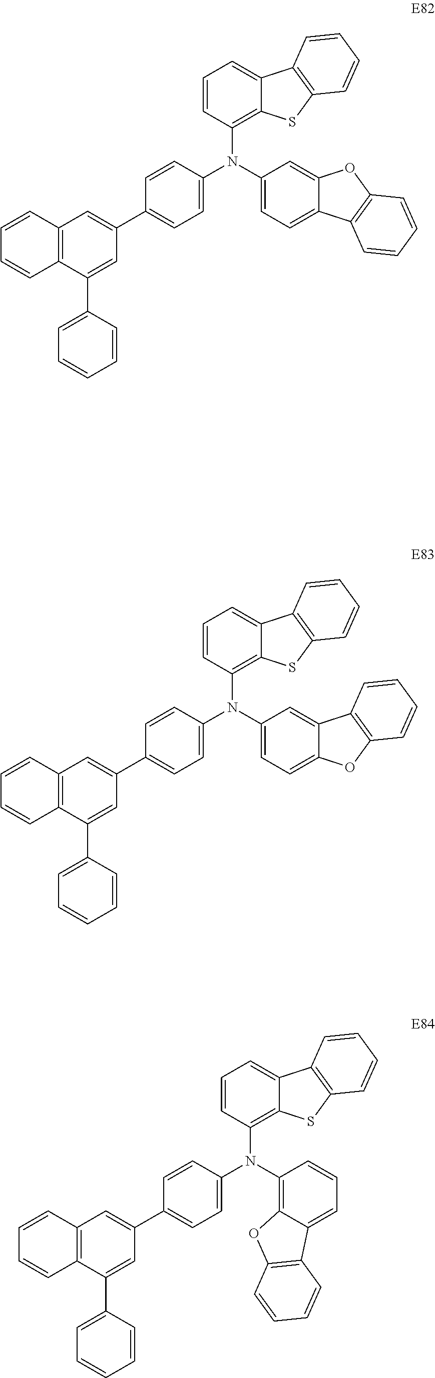 [SMILES: c1ccc(-c2cc(-c3ccc(N(c4ccc5c(c4)oc4ccccc45)c4cccc5c4sc4ccccc45)cc3)cc3ccccc23)cc1 ; c1ccc(-c2cc(-c3ccc(N(c4ccc5oc6ccccc6c5c4)c4cccc5c4sc4ccccc45)cc3)cc3ccccc23)cc1 ; c1ccc(-c2cc(-c3ccc(N(c4cccc5c4oc4ccccc45)c4cccc5c4sc4ccccc45)cc3)cc3ccccc23)cc1] 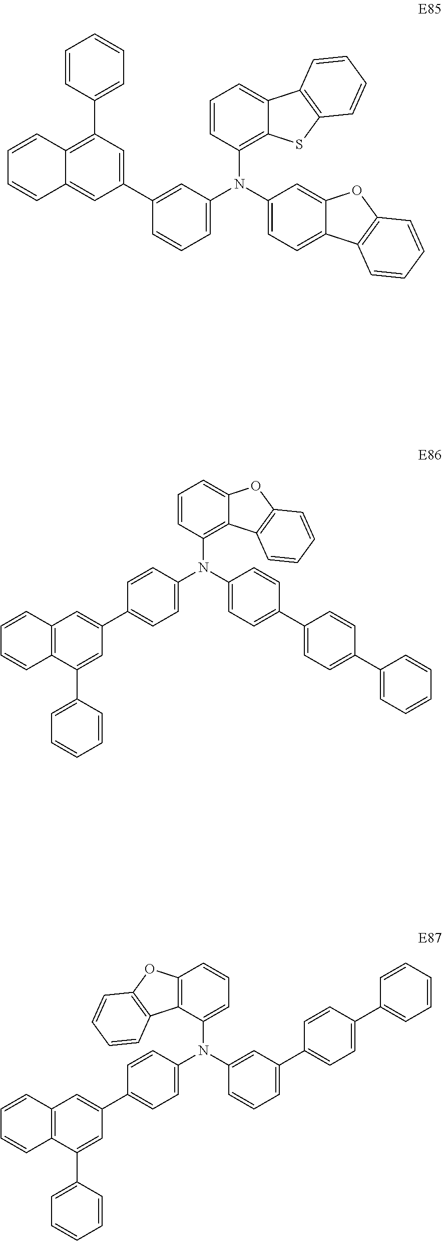 [SMILES: c1ccc(-c2cc(-c3cccc(N(c4ccc5c(c4)oc4ccccc45)c4cccc5c4sc4ccccc45)c3)cc3ccccc23)cc1 ; c1ccc(-c2ccc(-c3ccc(N(c4ccc(-c5cc(-c6ccccc6)c6ccccc6c5)cc4)c4cccc5oc6ccccc6c45)cc3)cc2)cc1 ; c1ccc(-c2ccc(-c3cccc(N(c4ccc(-c5cc(-c6ccccc6)c6ccccc6c5)cc4)c4cccc5oc6ccccc6c45)c3)cc2)cc1] 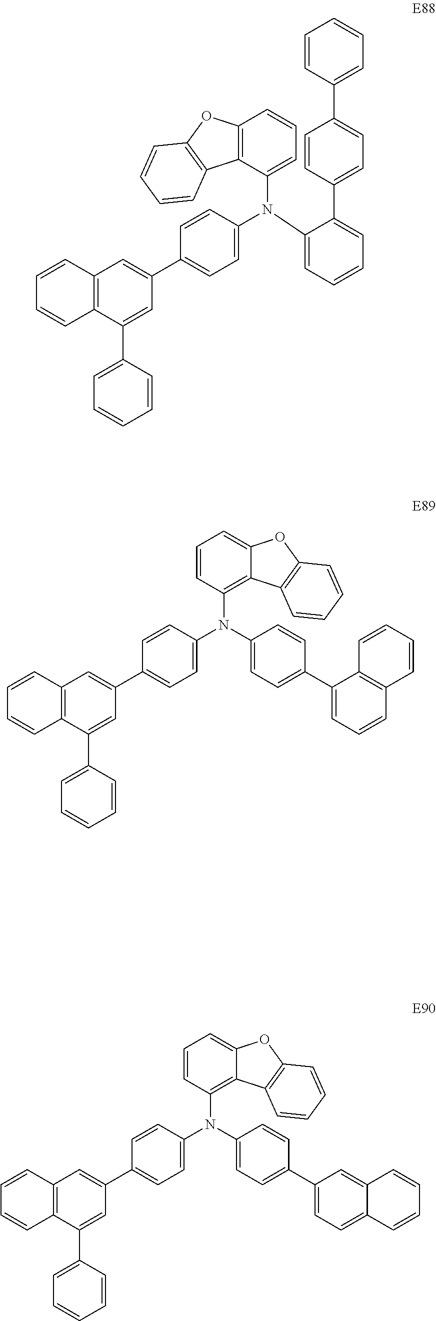 [SMILES: c1ccc(-c2ccc(-c3ccccc3N(c3ccc(-c4cc(-c5ccccc5)c5ccccc5c4)cc3)c3cccc4oc5ccccc5c34)cc2)cc1 ; c1ccc(-c2cc(-c3ccc(N(c4ccc(-c5cccc6ccccc56)cc4)c4cccc5oc6ccccc6c45)cc3)cc3ccccc23)cc1 ; c1ccc(-c2cc(-c3ccc(N(c4ccc(-c5ccc6ccccc6c5)cc4)c4cccc5oc6ccccc6c45)cc3)cc3ccccc23)cc1] 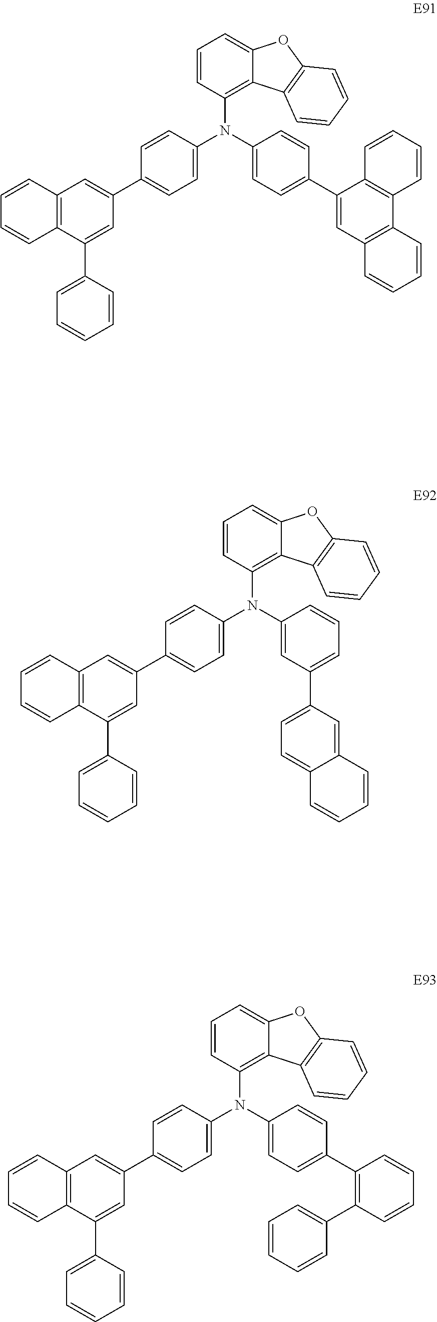 [SMILES: c1ccc(-c2cc(-c3ccc(N(c4ccc(-c5cc6ccccc6c6ccccc56)cc4)c4cccc5oc6ccccc6c45)cc3)cc3ccccc23)cc1 ; c1ccc(-c2cc(-c3ccc(N(c4cccc(-c5ccc6ccccc6c5)c4)c4cccc5oc6ccccc6c45)cc3)cc3ccccc23)cc1 ; c1ccc(-c2ccccc2-c2ccc(N(c3ccc(-c4cc(-c5ccccc5)c5ccccc5c4)cc3)c3cccc4oc5ccccc5c34)cc2)cc1] 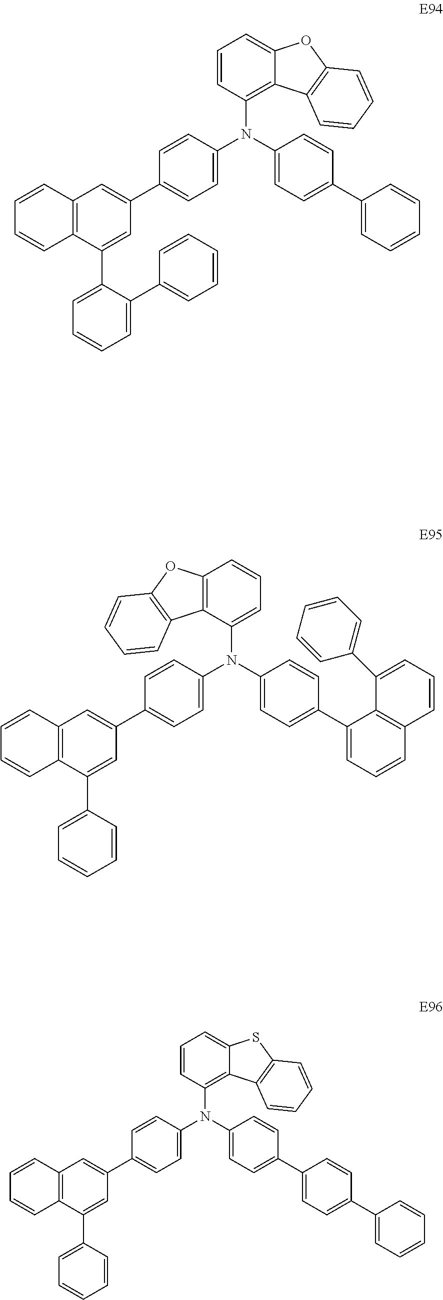 [SMILES: c1ccc(-c2ccc(N(c3ccc(-c4cc(-c5ccccc5-c5ccccc5)c5ccccc5c4)cc3)c3cccc4oc5ccccc5c34)cc2)cc1 ; c1ccc(-c2cc(-c3ccc(N(c4ccc(-c5cccc6cccc(-c7ccccc7)c56)cc4)c4cccc5oc6ccccc6c45)cc3)cc3ccccc23)cc1 ; c1ccc(-c2ccc(-c3ccc(N(c4ccc(-c5cc(-c6ccccc6)c6ccccc6c5)cc4)c4cccc5sc6ccccc6c45)cc3)cc2)cc1] 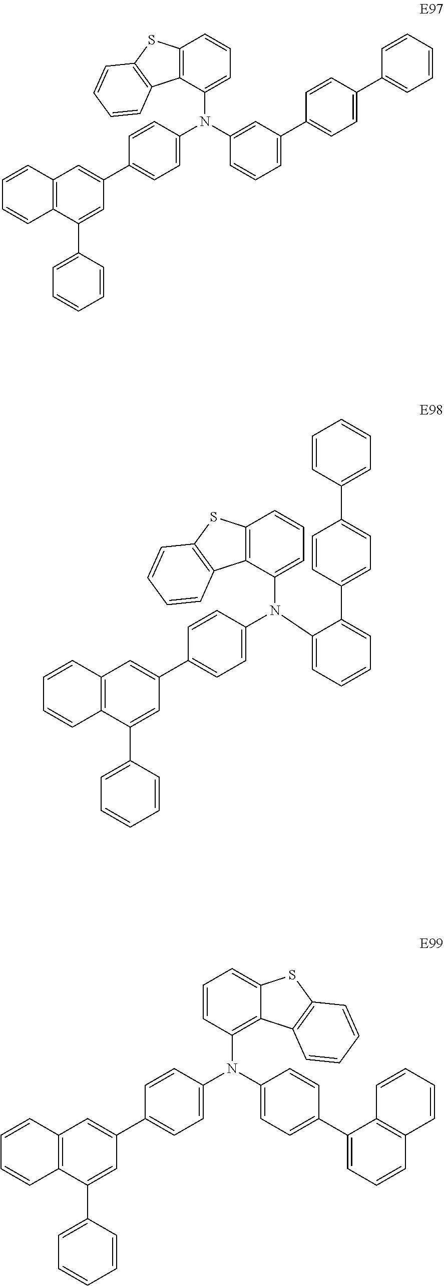 [SMILES: c1ccc(-c2ccc(-c3cccc(N(c4ccc(-c5cc(-c6ccccc6)c6ccccc6c5)cc4)c4cccc5sc6ccccc6c45)c3)cc2)cc1 ; c1ccc(-c2ccc(-c3ccccc3N(c3ccc(-c4cc(-c5ccccc5)c5ccccc5c4)cc3)c3cccc4sc5ccccc5c34)cc2)cc1 ; c1ccc(-c2cc(-c3ccc(N(c4ccc(-c5cccc6ccccc56)cc4)c4cccc5sc6ccccc6c45)cc3)cc3ccccc23)cc1] 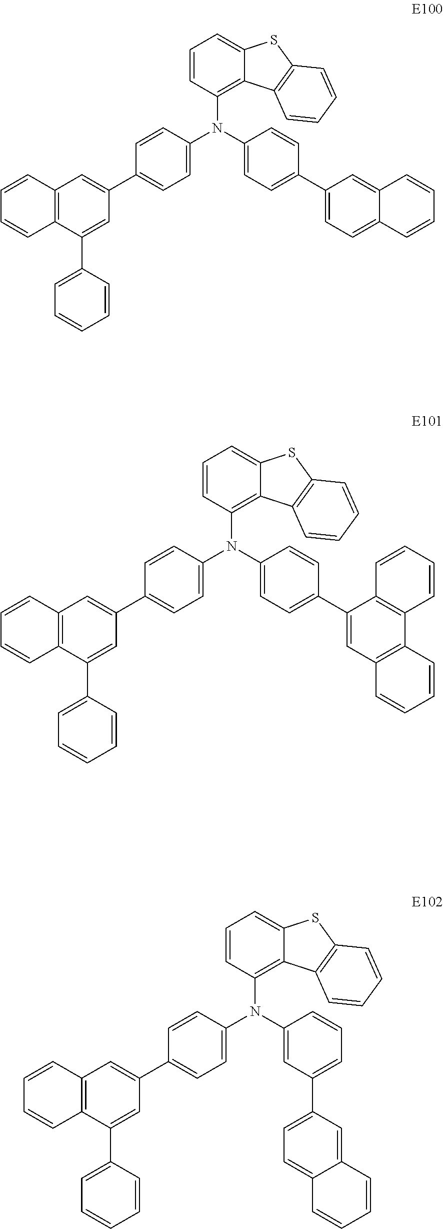 [SMILES: c1ccc(-c2cc(-c3ccc(N(c4ccc(-c5ccc6ccccc6c5)cc4)c4cccc5sc6ccccc6c45)cc3)cc3ccccc23)cc1 ; c1ccc(-c2cc(-c3ccc(N(c4ccc(-c5cc6ccccc6c6ccccc56)cc4)c4cccc5sc6ccccc6c45)cc3)cc3ccccc23)cc1 ; c1ccc(-c2cc(-c3ccc(N(c4cccc(-c5ccc6ccccc6c5)c4)c4cccc5sc6ccccc6c45)cc3)cc3ccccc23)cc1] 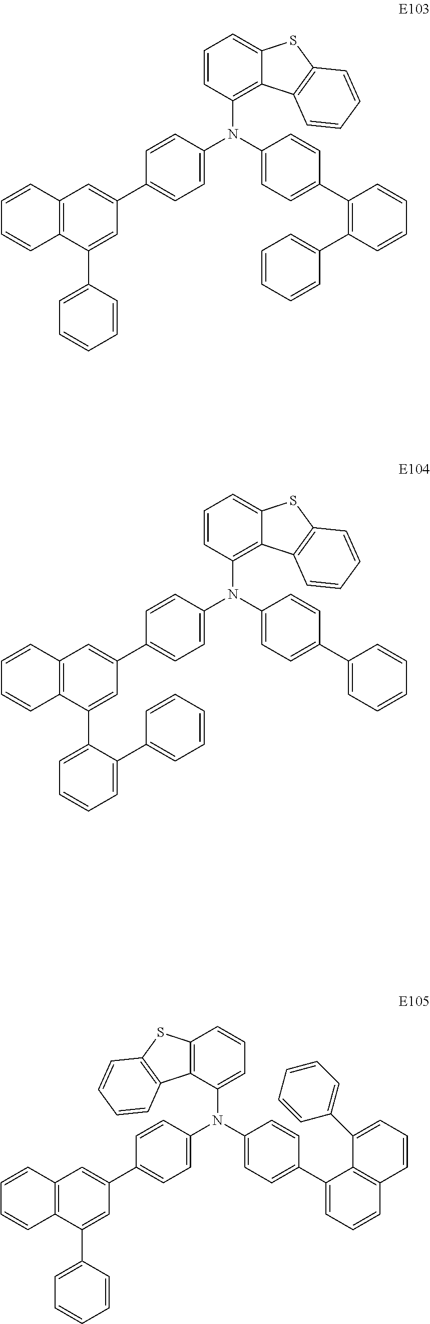 [SMILES: c1ccc(-c2ccccc2-c2ccc(N(c3ccc(-c4cc(-c5ccccc5)c5ccccc5c4)cc3)c3cccc4sc5ccccc5c34)cc2)cc1 ; c1ccc(-c2ccc(N(c3ccc(-c4cc(-c5ccccc5-c5ccccc5)c5ccccc5c4)cc3)c3cccc4sc5ccccc5c34)cc2)cc1 ; c1ccc(-c2cc(-c3ccc(N(c4ccc(-c5cccc6cccc(-c7ccccc7)c56)cc4)c4cccc5sc6ccccc6c45)cc3)cc3ccccc23)cc1] 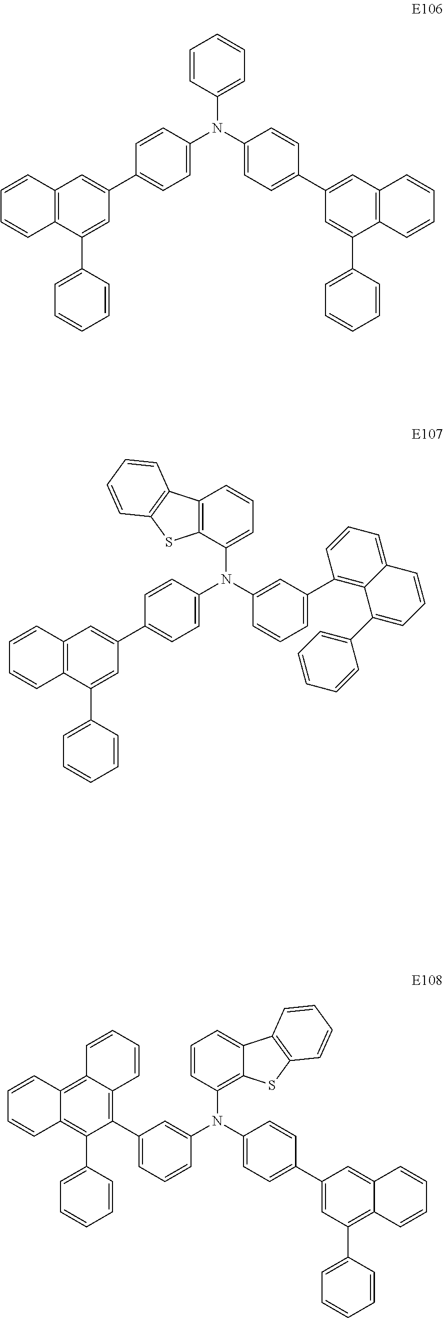 [SMILES: c1ccc(-c2cc(-c3ccc(N(c4ccccc4)c4ccc(-c5cc(-c6ccccc6)c6ccccc6c5)cc4)cc3)cc3ccccc23)cc1 ; c1ccc(-c2cc(-c3ccc(N(c4cccc(-c5cccc6cccc(-c7ccccc7)c56)c4)c4cccc5c4sc4ccccc45)cc3)cc3ccccc23)cc1 ; c1ccc(-c2cc(-c3ccc(N(c4cccc(-c5c(-c6ccccc6)c6ccccc6c6ccccc56)c4)c4cccc5c4sc4ccccc45)cc3)cc3ccccc23)cc1] 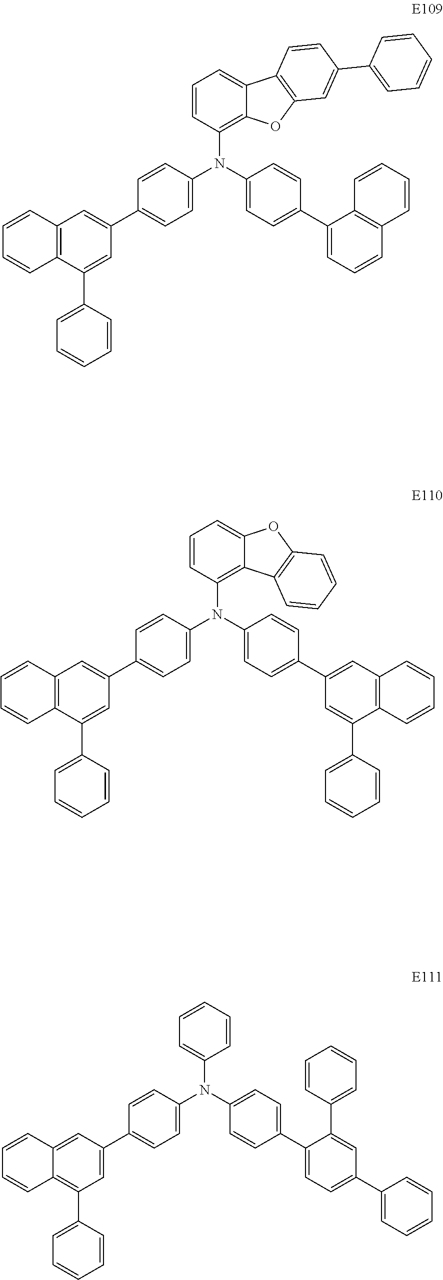 [SMILES: c1ccc(-c2ccc3c(c2)oc2c(N(c4ccc(-c5cc(-c6ccccc6)c6ccccc6c5)cc4)c4ccc(-c5cccc6ccccc56)cc4)cccc23)cc1 ; c1ccc(-c2cc(-c3ccc(N(c4ccc(-c5cc(-c6ccccc6)c6ccccc6c5)cc4)c4cccc5oc6ccccc6c45)cc3)cc3ccccc23)cc1 ; c1ccc(-c2ccc(-c3ccc(N(c4ccccc4)c4ccc(-c5cc(-c6ccccc6)c6ccccc6c5)cc4)cc3)c(-c3ccccc3)c2)cc1] 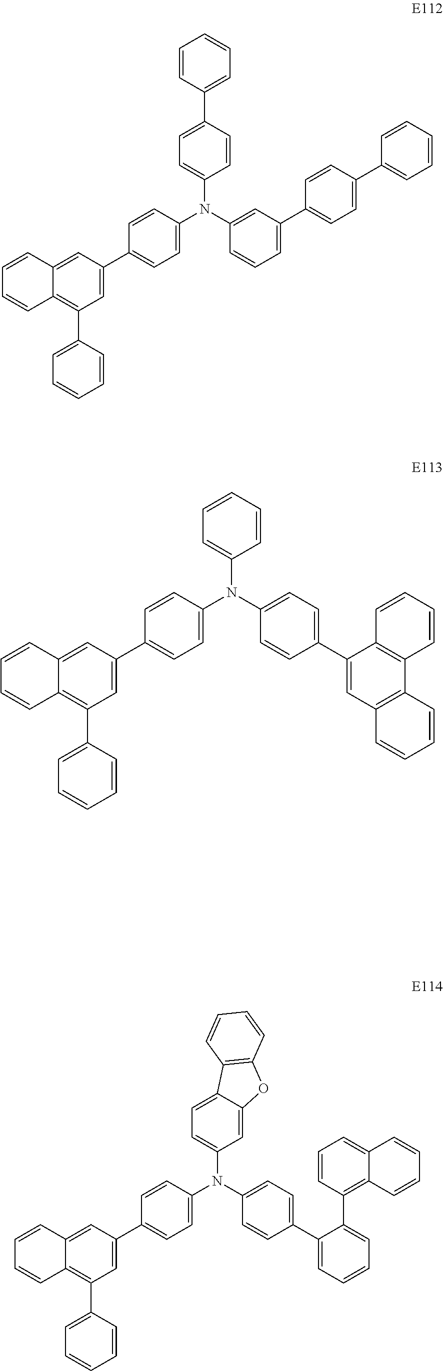 [SMILES: c1ccc(-c2ccc(-c3cccc(N(c4ccc(-c5ccccc5)cc4)c4ccc(-c5cc(-c6ccccc6)c6ccccc6c5)cc4)c3)cc2)cc1 ; c1ccc(-c2cc(-c3ccc(N(c4ccccc4)c4ccc(-c5cc6ccccc6c6ccccc56)cc4)cc3)cc3ccccc23)cc1 ; c1ccc(-c2cc(-c3ccc(N(c4ccc(-c5ccccc5-c5cccc6ccccc56)cc4)c4ccc5c(c4)oc4ccccc45)cc3)cc3ccccc23)cc1] 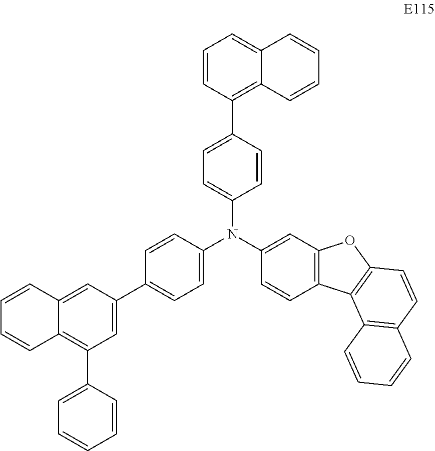 [SMILES: c1ccc(-c2cc(-c3ccc(N(c4ccc(-c5cccc6ccccc56)cc4)c4ccc5c(c4)oc4ccc6ccccc6c45)cc3)cc3ccccc23)cc1] 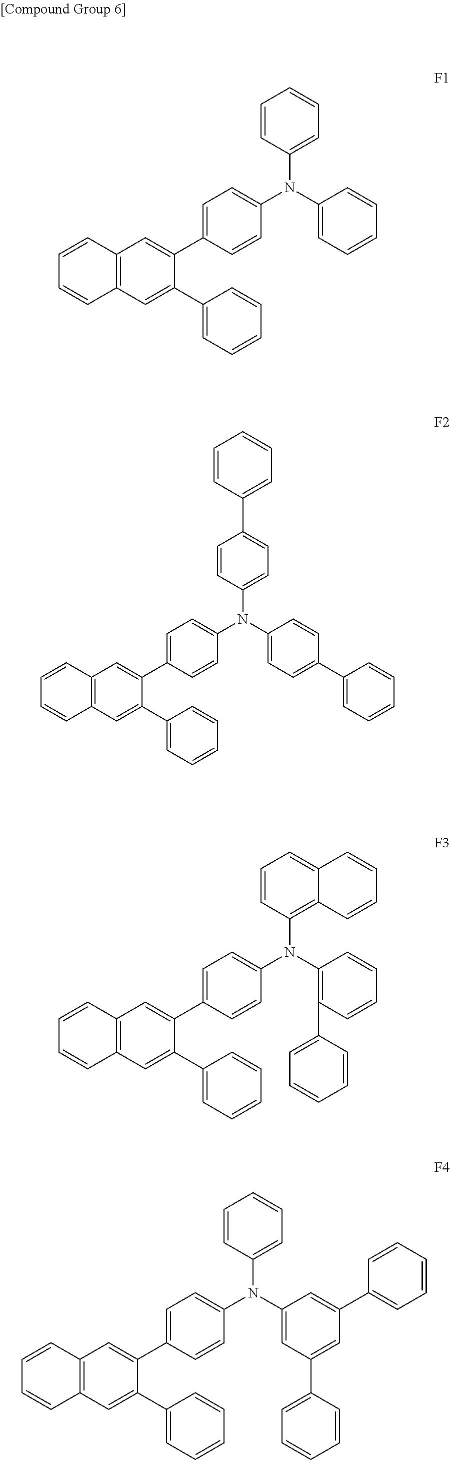 [SMILES: c1ccc(-c2cc3ccccc3cc2-c2ccc(N(c3ccccc3)c3ccccc3)cc2)cc1 ; c1ccc(-c2ccc(N(c3ccc(-c4ccccc4)cc3)c3ccc(-c4cc5ccccc5cc4-c4ccccc4)cc3)cc2)cc1 ; c1ccc(-c2cc3ccccc3cc2-c2ccc(N(c3ccccc3-c3ccccc3)c3cccc4ccccc34)cc2)cc1 ; c1ccc(-c2cc(-c3ccccc3)cc(N(c3ccccc3)c3ccc(-c4cc5ccccc5cc4-c4ccccc4)cc3)c2)cc1] 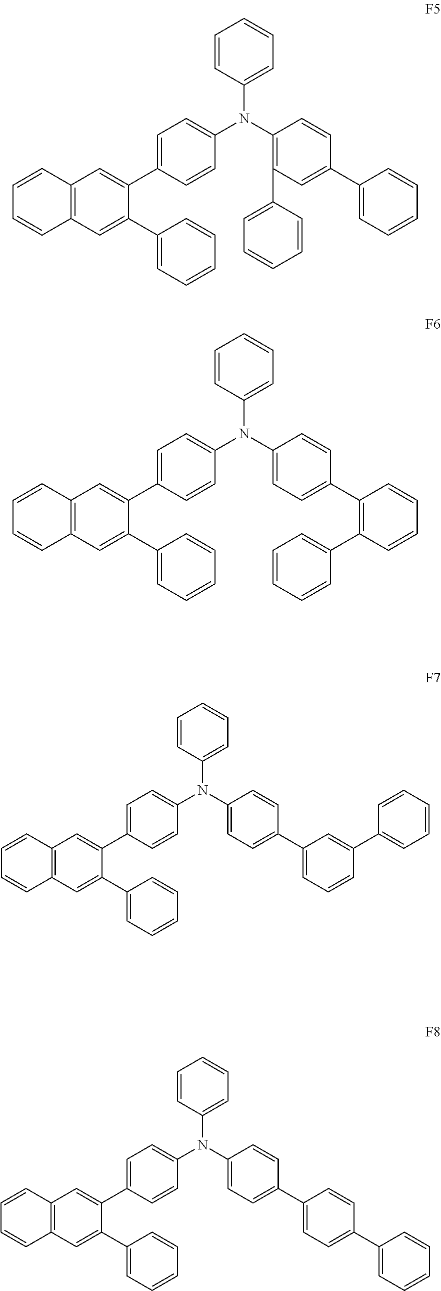 [SMILES: c1ccc(-c2ccc(N(c3ccccc3)c3ccc(-c4cc5ccccc5cc4-c4ccccc4)cc3)c(-c3ccccc3)c2)cc1 ; c1ccc(-c2ccccc2-c2ccc(N(c3ccccc3)c3ccc(-c4cc5ccccc5cc4-c4ccccc4)cc3)cc2)cc1 ; c1ccc(-c2cccc(-c3ccc(N(c4ccccc4)c4ccc(-c5cc6ccccc6cc5-c5ccccc5)cc4)cc3)c2)cc1 ; c1ccc(-c2ccc(-c3ccc(N(c4ccccc4)c4ccc(-c5cc6ccccc6cc5-c5ccccc5)cc4)cc3)cc2)cc1] 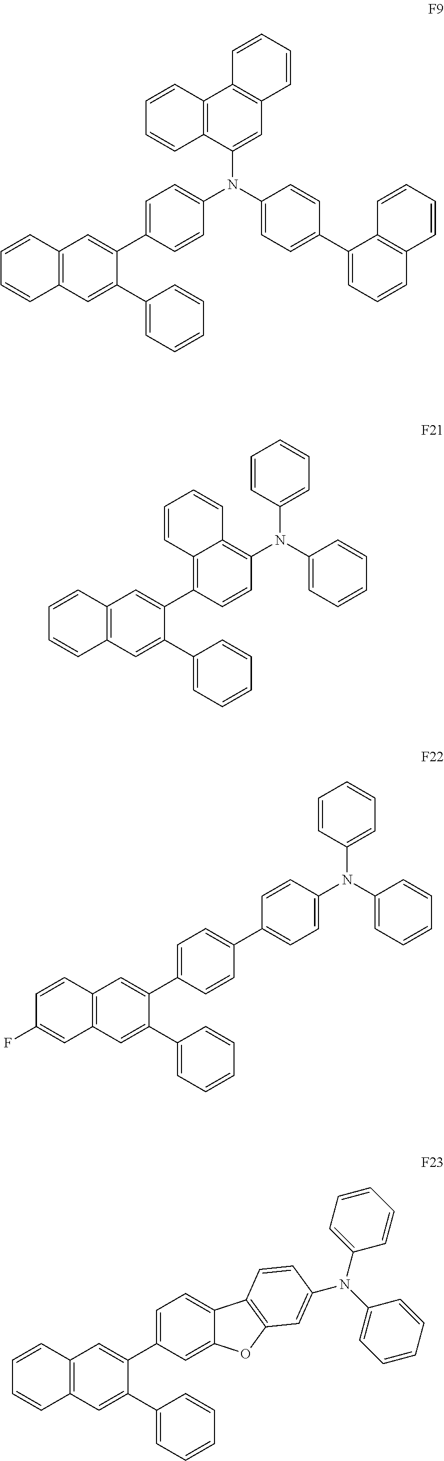 [SMILES: c1ccc(-c2cc3ccccc3cc2-c2ccc(N(c3ccc(-c4cccc5ccccc45)cc3)c3cc4ccccc4c4ccccc34)cc2)cc1 ; c1ccc(-c2cc3ccccc3cc2-c2ccc(N(c3ccccc3)c3ccccc3)c3ccccc23)cc1 ; Fc1ccc2cc(-c3ccc(-c4ccc(N(c5ccccc5)c5ccccc5)cc4)cc3)c(-c3ccccc3)cc2c1 ; c1ccc(-c2cc3ccccc3cc2-c2ccc3c(c2)oc2cc(N(c4ccccc4)c4ccccc4)ccc23)cc1] 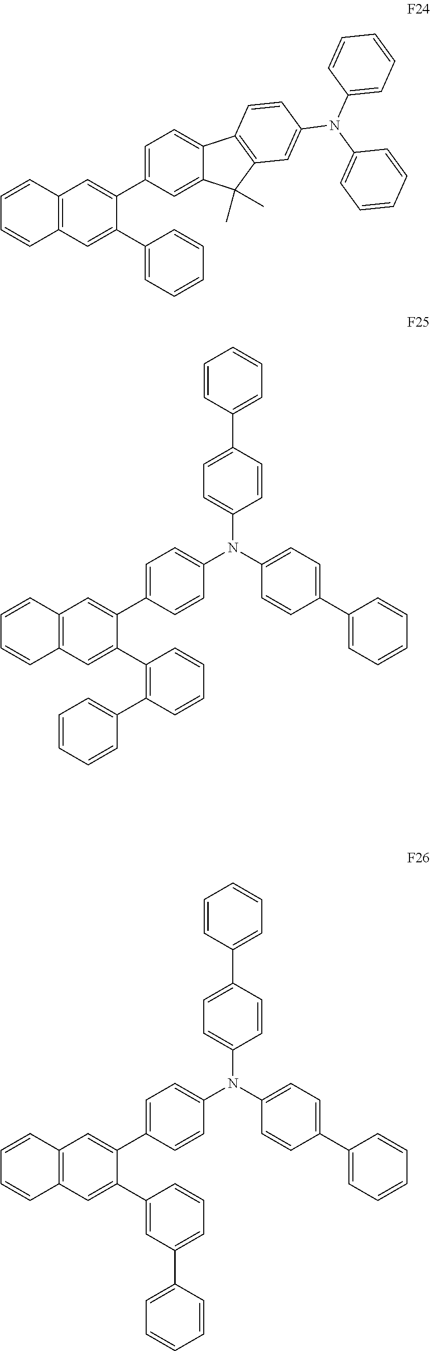 [SMILES: CC1(C)c2cc(-c3cc4ccccc4cc3-c3ccccc3)ccc2-c2ccc(N(c3ccccc3)c3ccccc3)cc21 ; c1ccc(-c2ccc(N(c3ccc(-c4ccccc4)cc3)c3ccc(-c4cc5ccccc5cc4-c4ccccc4-c4ccccc4)cc3)cc2)cc1 ; c1ccc(-c2ccc(N(c3ccc(-c4ccccc4)cc3)c3ccc(-c4cc5ccccc5cc4-c4cccc(-c5ccccc5)c4)cc3)cc2)cc1] 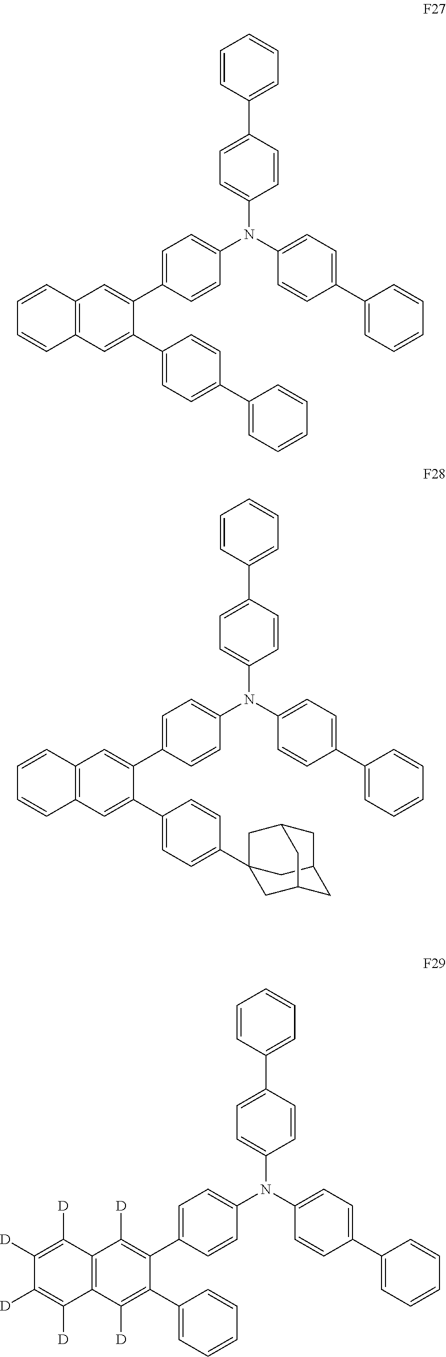 [SMILES: c1ccc(-c2ccc(-c3cc4ccccc4cc3-c3ccc(N(c4ccc(-c5ccccc5)cc4)c4ccc(-c5ccccc5)cc4)cc3)cc2)cc1 ; c1ccc(-c2ccc(N(c3ccc(-c4ccccc4)cc3)c3ccc(-c4cc5ccccc5cc4-c4ccc(C56CC7CC(CC(C7)C5)C6)cc4)cc3)cc2)cc1 ; [2H]c1c([2H])c([2H])c2c([2H])c(-c3ccc(N(c4ccc(-c5ccccc5)cc4)c4ccc(-c5ccccc5)cc4)cc3)c(-c3ccccc3)c([2H])c2c1[2H]] 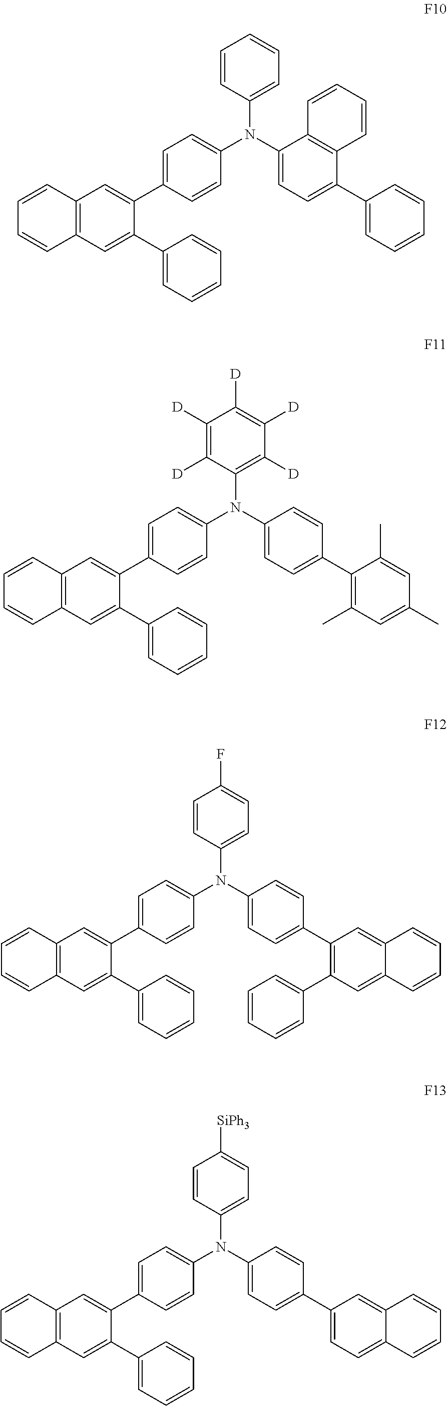 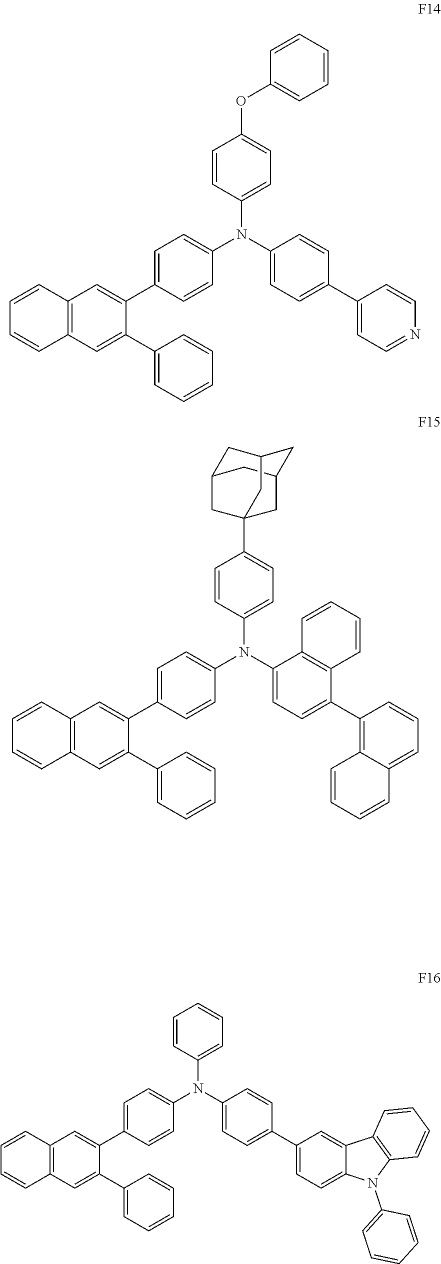 [SMILES: c1ccc(Oc2ccc(N(c3ccc(-c4ccncc4)cc3)c3ccc(-c4cc5ccccc5cc4-c4ccccc4)cc3)cc2)cc1 ; c1ccc(-c2cc3ccccc3cc2-c2ccc(N(c3ccc(C45CC6CC(CC(C6)C4)C5)cc3)c3ccc(-c4cccc5ccccc45)c4ccccc34)cc2)cc1 ; c1ccc(-c2cc3ccccc3cc2-c2ccc(N(c3ccccc3)c3ccc(-c4ccc5c(c4)c4ccccc4n5-c4ccccc4)cc3)cc2)cc1] 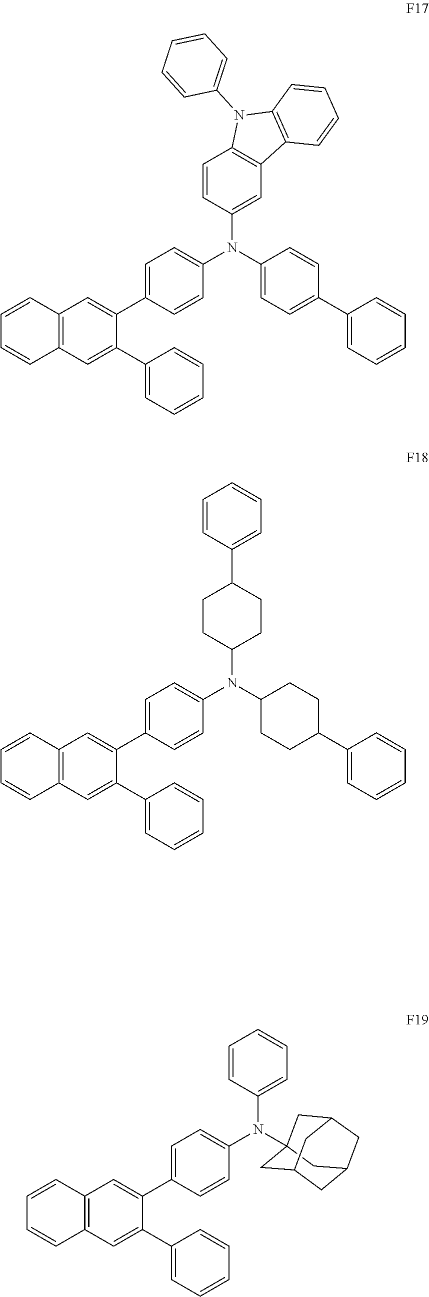 [SMILES: c1ccc(-c2ccc(N(c3ccc(-c4cc5ccccc5cc4-c4ccccc4)cc3)c3ccc4c(c3)c3ccccc3n4-c3ccccc3)cc2)cc1 ; c1ccc(-c2cc3ccccc3cc2-c2ccc(N(C3CCC(c4ccccc4)CC3)C3CCC(c4ccccc4)CC3)cc2)cc1 ; c1ccc(-c2cc3ccccc3cc2-c2ccc(N(c3ccccc3)C34CC5CC(CC(C5)C3)C4)cc2)cc1] 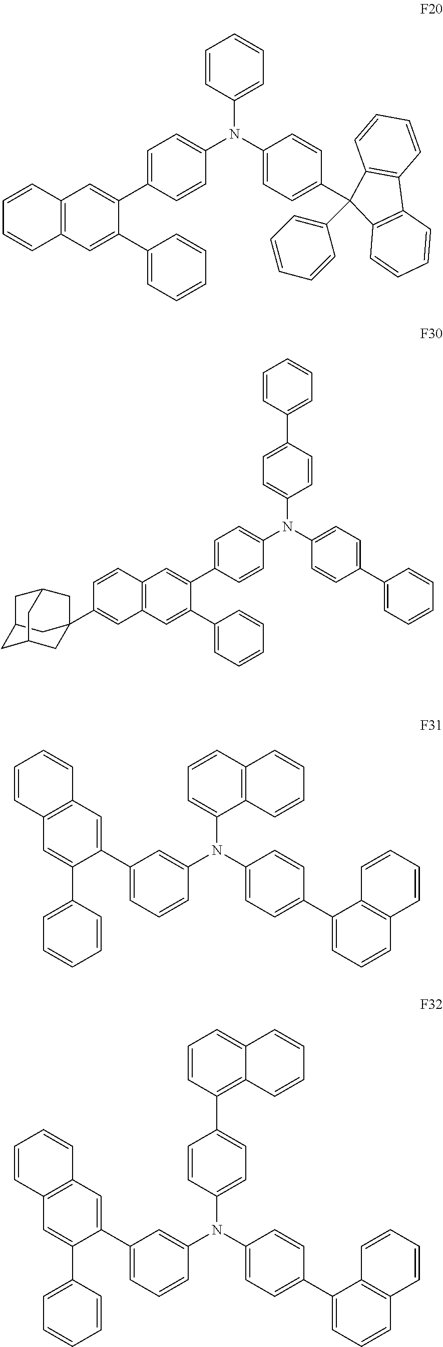 [SMILES: c1ccc(-c2cc3ccccc3cc2-c2ccc(N(c3ccccc3)c3ccc(C4(c5ccccc5)c5ccccc5-c5ccccc54)cc3)cc2)cc1 ; c1ccc(-c2ccc(N(c3ccc(-c4ccccc4)cc3)c3ccc(-c4cc5ccc(C67CC8CC(CC(C8)C6)C7)cc5cc4-c4ccccc4)cc3)cc2)cc1 ; c1ccc(-c2cc3ccccc3cc2-c2cccc(N(c3ccc(-c4cccc5ccccc45)cc3)c3cccc4ccccc34)c2)cc1 ; c1ccc(-c2cc3ccccc3cc2-c2cccc(N(c3ccc(-c4cccc5ccccc45)cc3)c3ccc(-c4cccc5ccccc45)cc3)c2)cc1] 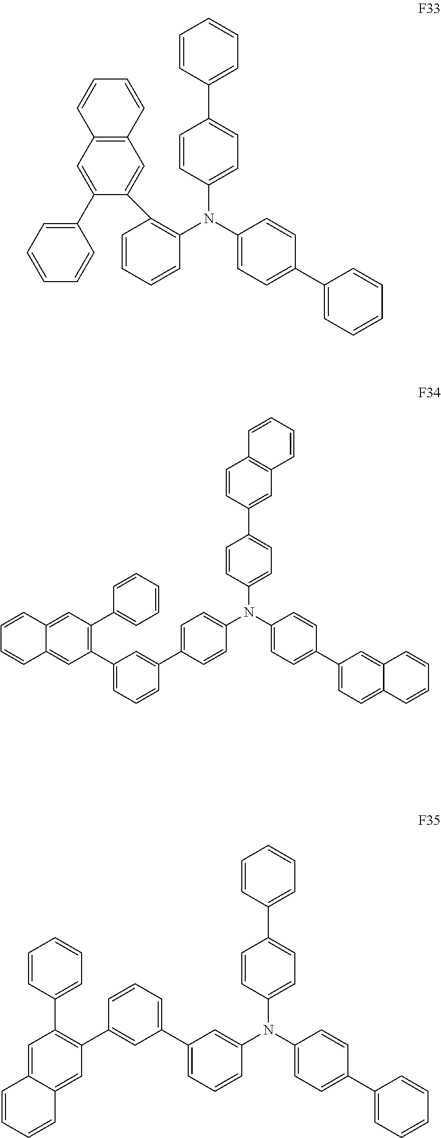 [SMILES: c1ccc(-c2ccc(N(c3ccc(-c4ccccc4)cc3)c3ccccc3-c3cc4ccccc4cc3-c3ccccc3)cc2)cc1 ; c1ccc(-c2cc3ccccc3cc2-c2cccc(-c3ccc(N(c4ccc(-c5ccc6ccccc6c5)cc4)c4ccc(-c5ccc6ccccc6c5)cc4)cc3)c2)cc1 ; c1ccc(-c2ccc(N(c3ccc(-c4ccccc4)cc3)c3cccc(-c4cccc(-c5cc6ccccc6cc5-c5ccccc5)c4)c3)cc2)cc1] 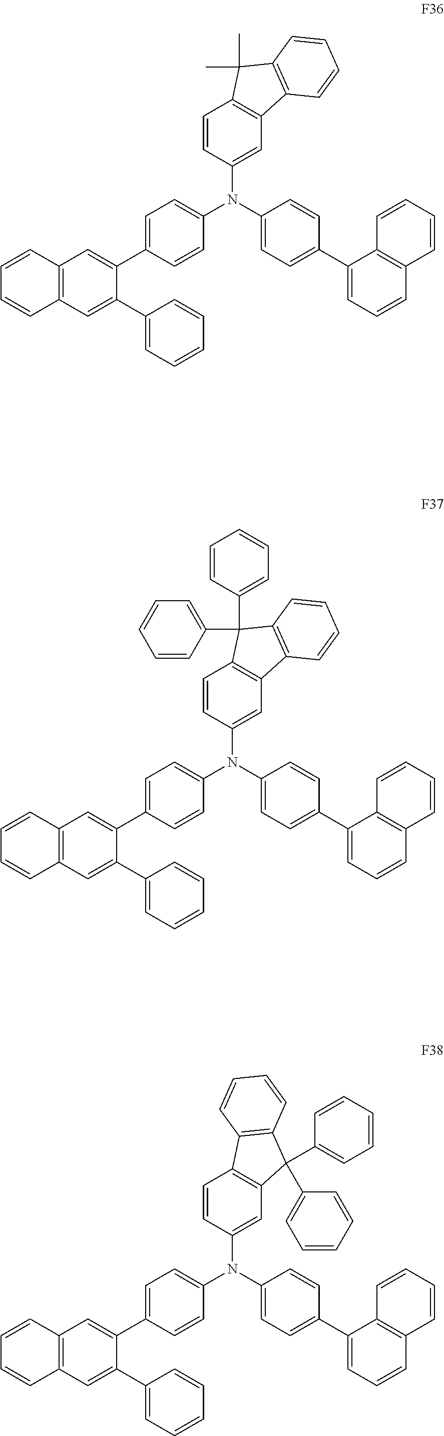 [SMILES: CC1(C)c2ccccc2-c2cc(N(c3ccc(-c4cc5ccccc5cc4-c4ccccc4)cc3)c3ccc(-c4cccc5ccccc45)cc3)ccc21 ; c1ccc(-c2cc3ccccc3cc2-c2ccc(N(c3ccc(-c4cccc5ccccc45)cc3)c3ccc4c(c3)-c3ccccc3C4(c3ccccc3)c3ccccc3)cc2)cc1 ; c1ccc(-c2cc3ccccc3cc2-c2ccc(N(c3ccc(-c4cccc5ccccc45)cc3)c3ccc4c(c3)C(c3ccccc3)(c3ccccc3)c3ccccc3-4)cc2)cc1] 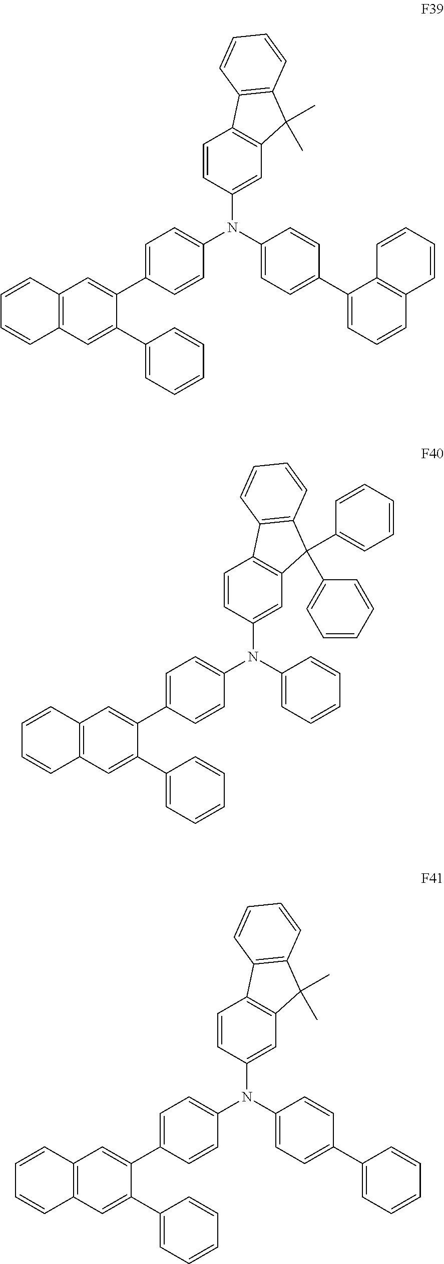 [SMILES: CC1(C)c2ccccc2-c2ccc(N(c3ccc(-c4cc5ccccc5cc4-c4ccccc4)cc3)c3ccc(-c4cccc5ccccc45)cc3)cc21 ; c1ccc(-c2cc3ccccc3cc2-c2ccc(N(c3ccccc3)c3ccc4c(c3)C(c3ccccc3)(c3ccccc3)c3ccccc3-4)cc2)cc1 ; CC1(C)c2ccccc2-c2ccc(N(c3ccc(-c4ccccc4)cc3)c3ccc(-c4cc5ccccc5cc4-c4ccccc4)cc3)cc21] 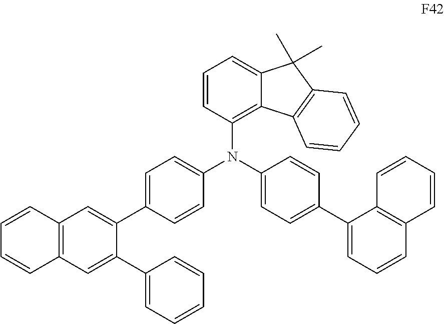 [SMILES: CC1(C)c2ccccc2-c2c(N(c3ccc(-c4cc5ccccc5cc4-c4ccccc4)cc3)c3ccc(-c4cccc5ccccc45)cc3)cccc21] 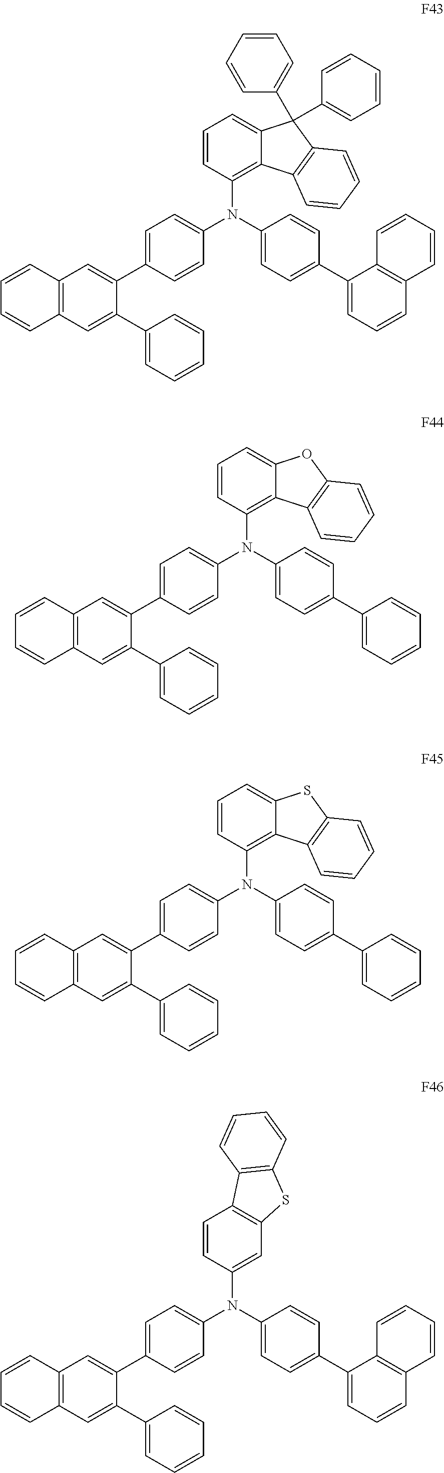 [SMILES: c1ccc(-c2cc3ccccc3cc2-c2ccc(N(c3ccc(-c4cccc5ccccc45)cc3)c3cccc4c3-c3ccccc3C4(c3ccccc3)c3ccccc3)cc2)cc1 ; c1ccc(-c2ccc(N(c3ccc(-c4cc5ccccc5cc4-c4ccccc4)cc3)c3cccc4oc5ccccc5c34)cc2)cc1 ; c1ccc(-c2ccc(N(c3ccc(-c4cc5ccccc5cc4-c4ccccc4)cc3)c3cccc4sc5ccccc5c34)cc2)cc1 ; c1ccc(-c2cc3ccccc3cc2-c2ccc(N(c3ccc(-c4cccc5ccccc45)cc3)c3ccc4c(c3)sc3ccccc34)cc2)cc1] 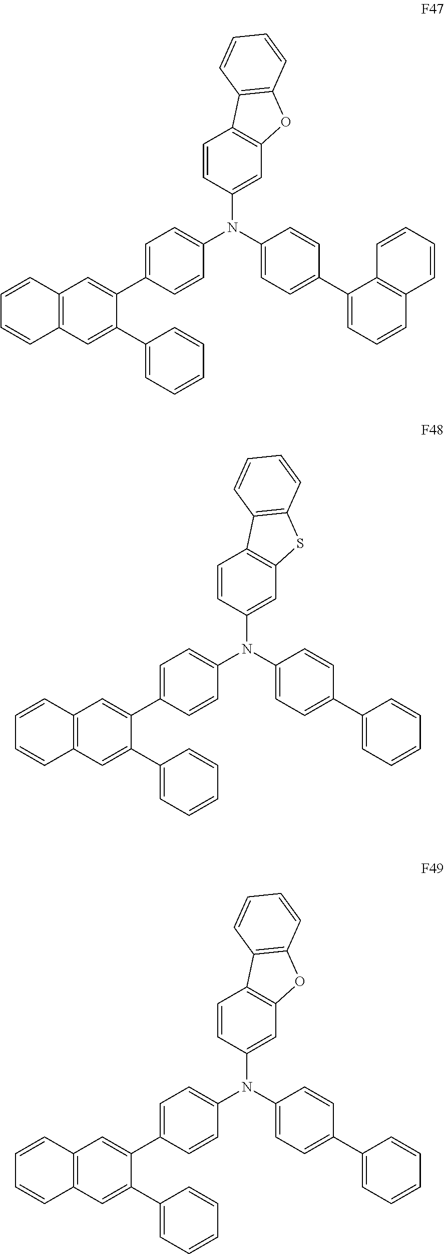 [SMILES: c1ccc(-c2cc3ccccc3cc2-c2ccc(N(c3ccc(-c4cccc5ccccc45)cc3)c3ccc4c(c3)oc3ccccc34)cc2)cc1 ; c1ccc(-c2ccc(N(c3ccc(-c4cc5ccccc5cc4-c4ccccc4)cc3)c3ccc4c(c3)sc3ccccc34)cc2)cc1 ; c1ccc(-c2ccc(N(c3ccc(-c4cc5ccccc5cc4-c4ccccc4)cc3)c3ccc4c(c3)oc3ccccc34)cc2)cc1] 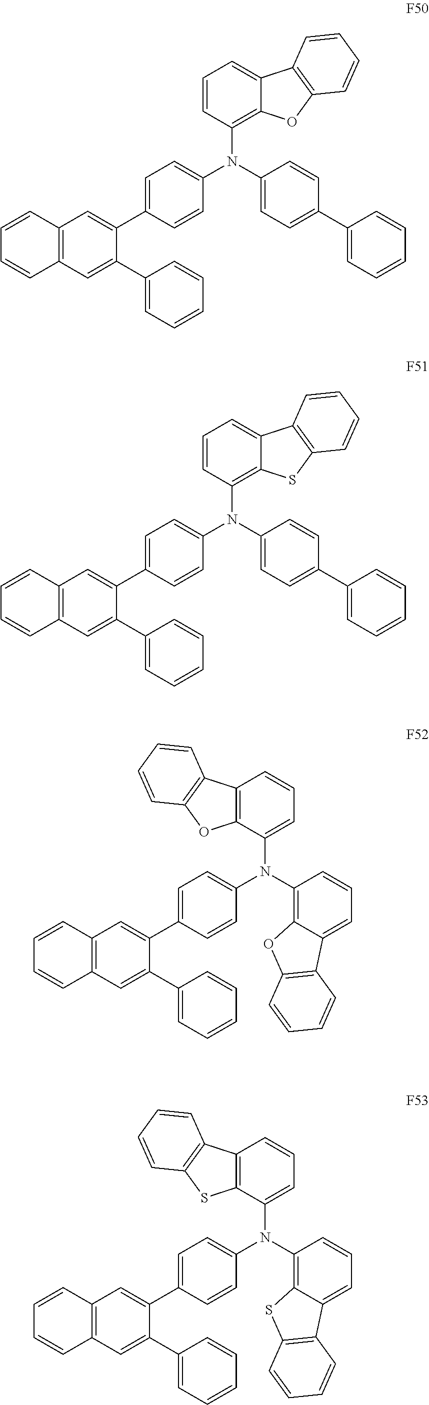 [SMILES: c1ccc(-c2ccc(N(c3ccc(-c4cc5ccccc5cc4-c4ccccc4)cc3)c3cccc4c3oc3ccccc34)cc2)cc1 ; c1ccc(-c2ccc(N(c3ccc(-c4cc5ccccc5cc4-c4ccccc4)cc3)c3cccc4c3sc3ccccc34)cc2)cc1 ; c1ccc(-c2cc3ccccc3cc2-c2ccc(N(c3cccc4c3oc3ccccc34)c3cccc4c3oc3ccccc34)cc2)cc1 ; c1ccc(-c2cc3ccccc3cc2-c2ccc(N(c3cccc4c3sc3ccccc34)c3cccc4c3sc3ccccc34)cc2)cc1] 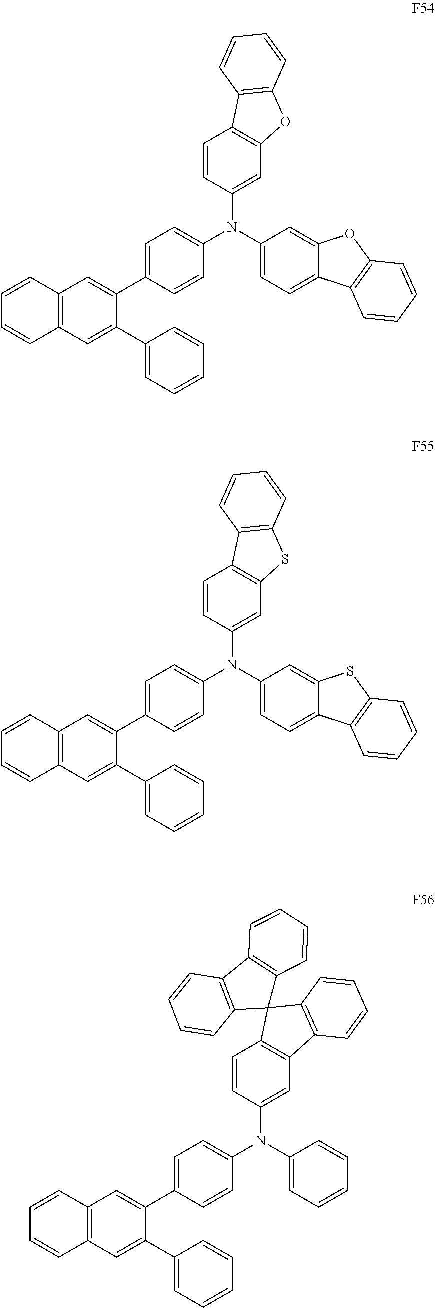 [SMILES: c1ccc(-c2cc3ccccc3cc2-c2ccc(N(c3ccc4c(c3)oc3ccccc34)c3ccc4c(c3)oc3ccccc34)cc2)cc1 ; c1ccc(-c2cc3ccccc3cc2-c2ccc(N(c3ccc4c(c3)sc3ccccc34)c3ccc4c(c3)sc3ccccc34)cc2)cc1 ; c1ccc(-c2cc3ccccc3cc2-c2ccc(N(c3ccccc3)c3ccc4c(c3)-c3ccccc3C43c4ccccc4-c4ccccc43)cc2)cc1] 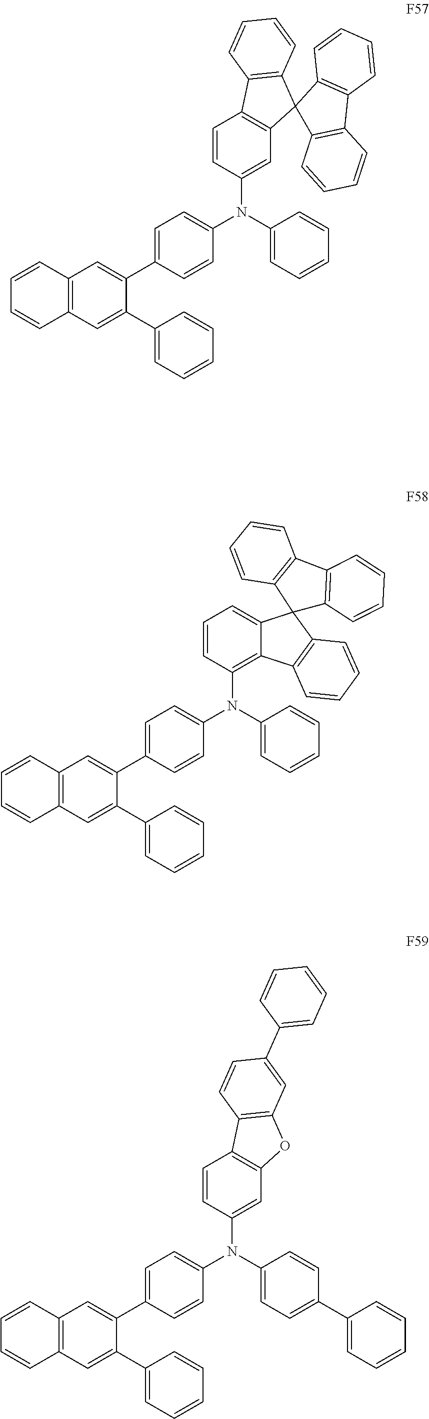 [SMILES: c1ccc(-c2cc3ccccc3cc2-c2ccc(N(c3ccccc3)c3ccc4c(c3)C3(c5ccccc5-c5ccccc53)c3ccccc3-4)cc2)cc1 ; c1ccc(-c2cc3ccccc3cc2-c2ccc(N(c3ccccc3)c3cccc4c3-c3ccccc3C43c4ccccc4-c4ccccc43)cc2)cc1 ; c1ccc(-c2ccc(N(c3ccc(-c4cc5ccccc5cc4-c4ccccc4)cc3)c3ccc4c(c3)oc3cc(-c5ccccc5)ccc34)cc2)cc1] 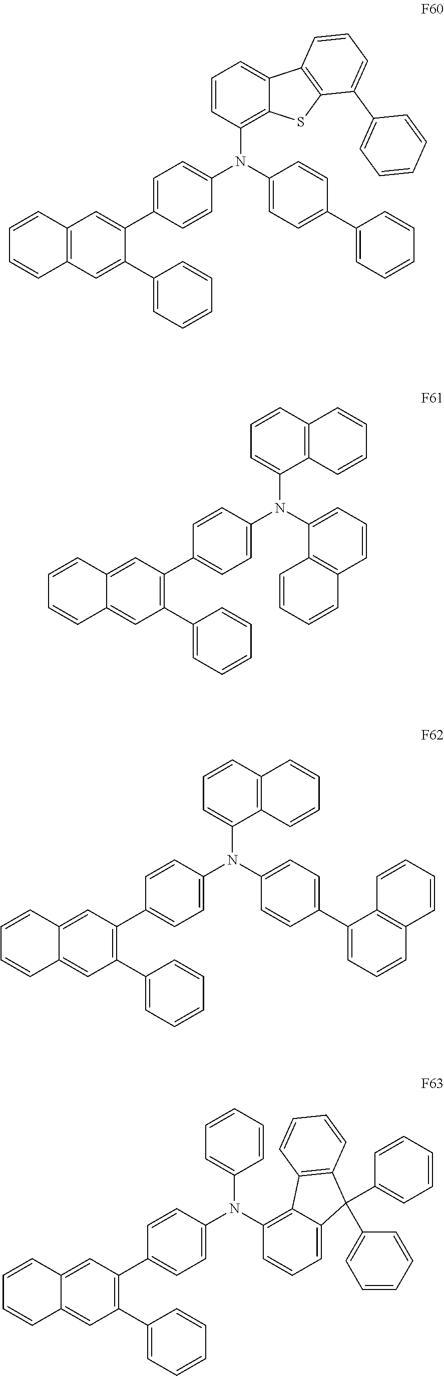 [SMILES: c1ccc(-c2ccc(N(c3ccc(-c4cc5ccccc5cc4-c4ccccc4)cc3)c3cccc4c3sc3c(-c5ccccc5)cccc34)cc2)cc1 ; c1ccc(-c2cc3ccccc3cc2-c2ccc(N(c3cccc4ccccc34)c3cccc4ccccc34)cc2)cc1 ; c1ccc(-c2cc3ccccc3cc2-c2ccc(N(c3ccc(-c4cccc5ccccc45)cc3)c3cccc4ccccc34)cc2)cc1 ; c1ccc(-c2cc3ccccc3cc2-c2ccc(N(c3ccccc3)c3cccc4c3-c3ccccc3C4(c3ccccc3)c3ccccc3)cc2)cc1] 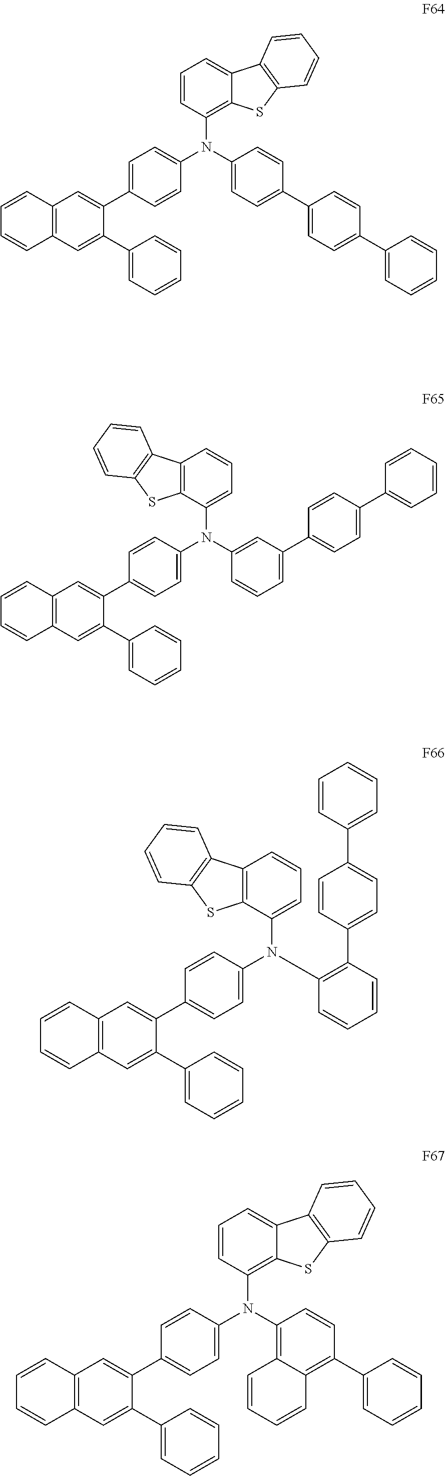 [SMILES: c1ccc(-c2ccc(-c3ccc(N(c4ccc(-c5cc6ccccc6cc5-c5ccccc5)cc4)c4cccc5c4sc4ccccc45)cc3)cc2)cc1 ; c1ccc(-c2ccc(-c3cccc(N(c4ccc(-c5cc6ccccc6cc5-c5ccccc5)cc4)c4cccc5c4sc4ccccc45)c3)cc2)cc1 ; c1ccc(-c2ccc(-c3ccccc3N(c3ccc(-c4cc5ccccc5cc4-c4ccccc4)cc3)c3cccc4c3sc3ccccc34)cc2)cc1 ; c1ccc(-c2cc3ccccc3cc2-c2ccc(N(c3ccc(-c4ccccc4)c4ccccc34)c3cccc4c3sc3ccccc34)cc2)cc1] 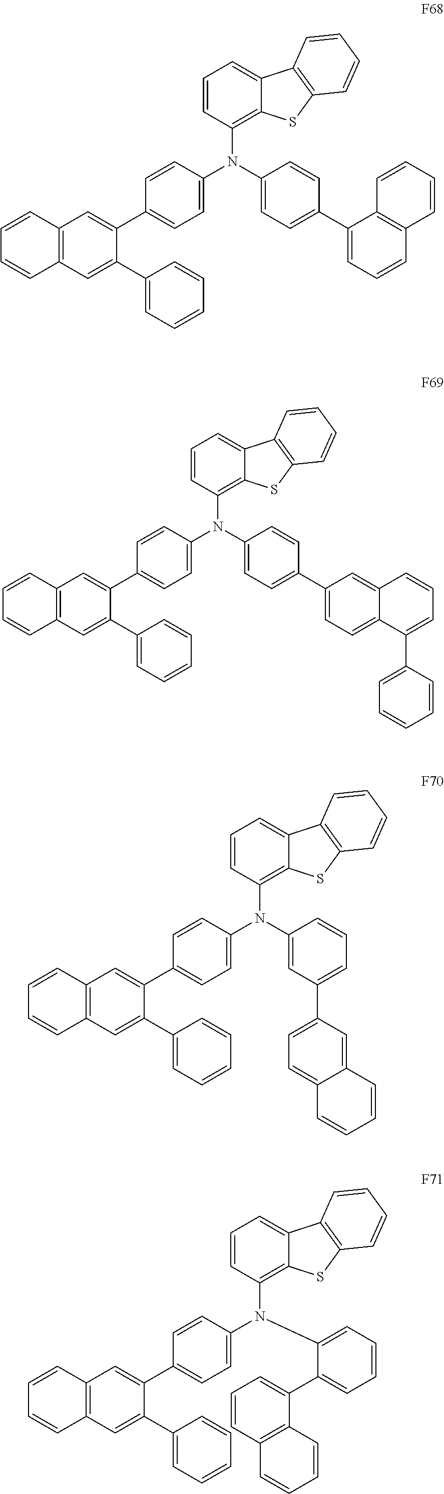 [SMILES: c1ccc(-c2cc3ccccc3cc2-c2ccc(N(c3ccc(-c4cccc5ccccc45)cc3)c3cccc4c3sc3ccccc34)cc2)cc1 ; c1ccc(-c2cc3ccccc3cc2-c2ccc(N(c3ccc(-c4ccc5c(-c6ccccc6)cccc5c4)cc3)c3cccc4c3sc3ccccc34)cc2)cc1 ; c1ccc(-c2cc3ccccc3cc2-c2ccc(N(c3cccc(-c4ccc5ccccc5c4)c3)c3cccc4c3sc3ccccc34)cc2)cc1 ; c1ccc(-c2cc3ccccc3cc2-c2ccc(N(c3ccccc3-c3cccc4ccccc34)c3cccc4c3sc3ccccc34)cc2)cc1] 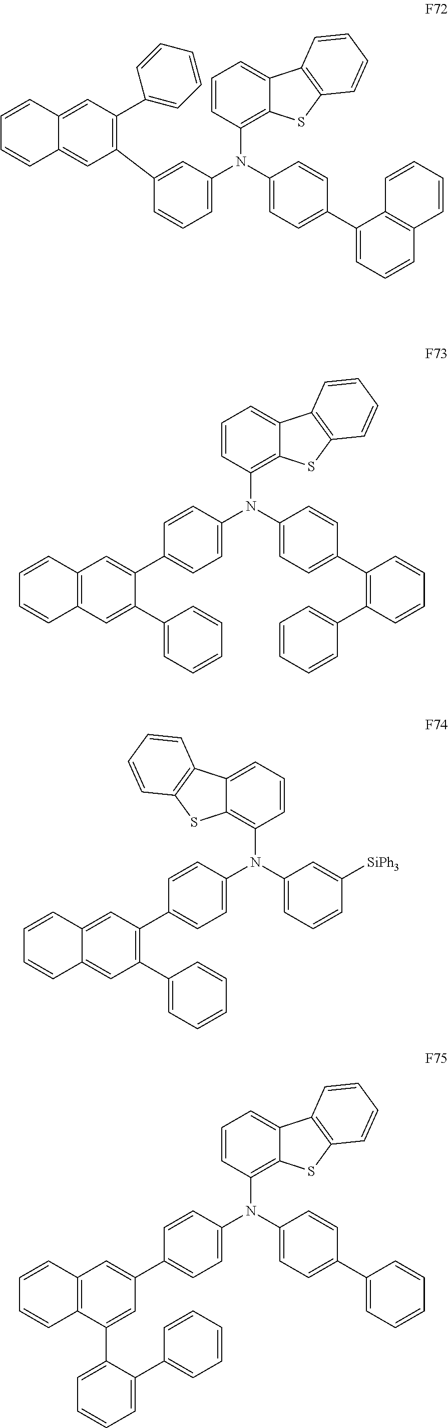 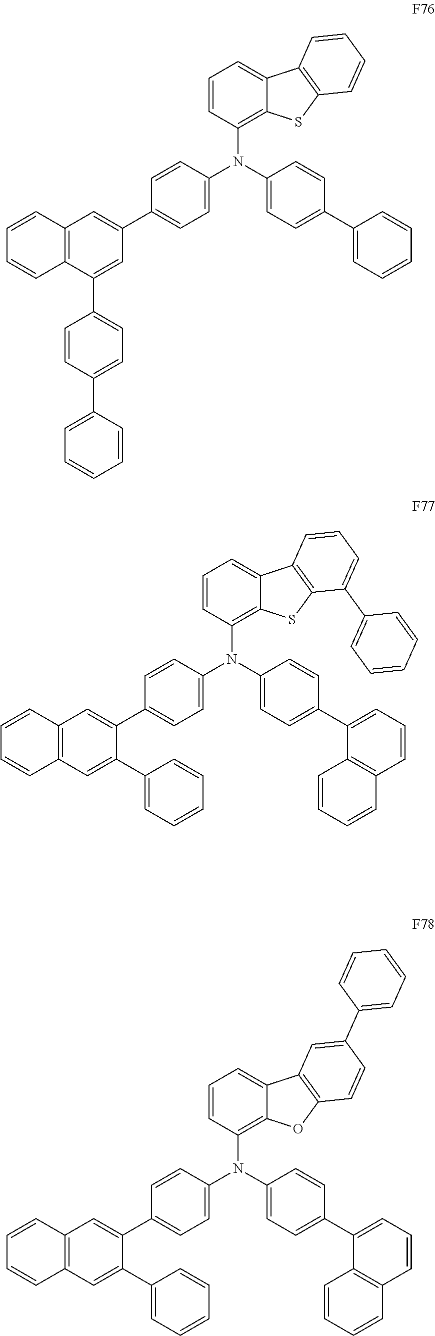 [SMILES: c1ccc(-c2ccc(-c3cc(-c4ccc(N(c5ccc(-c6ccccc6)cc5)c5cccc6c5sc5ccccc56)cc4)cc4ccccc34)cc2)cc1 ; c1ccc(-c2cc3ccccc3cc2-c2ccc(N(c3ccc(-c4cccc5ccccc45)cc3)c3cccc4c3sc3c(-c5ccccc5)cccc34)cc2)cc1 ; c1ccc(-c2ccc3oc4c(N(c5ccc(-c6cc7ccccc7cc6-c6ccccc6)cc5)c5ccc(-c6cccc7ccccc67)cc5)cccc4c3c2)cc1] 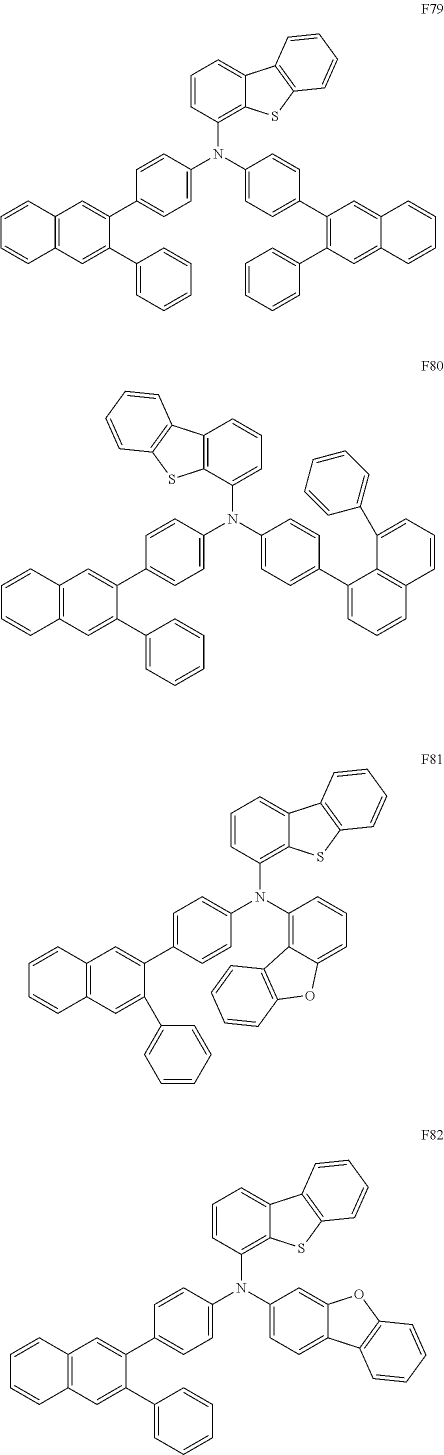 [SMILES: c1ccc(-c2cc3ccccc3cc2-c2ccc(N(c3ccc(-c4cc5ccccc5cc4-c4ccccc4)cc3)c3cccc4c3sc3ccccc34)cc2)cc1 ; c1ccc(-c2cc3ccccc3cc2-c2ccc(N(c3ccc(-c4cccc5cccc(-c6ccccc6)c45)cc3)c3cccc4c3sc3ccccc34)cc2)cc1 ; c1ccc(-c2cc3ccccc3cc2-c2ccc(N(c3cccc4c3sc3ccccc34)c3cccc4oc5ccccc5c34)cc2)cc1 ; c1ccc(-c2cc3ccccc3cc2-c2ccc(N(c3ccc4c(c3)oc3ccccc34)c3cccc4c3sc3ccccc34)cc2)cc1] 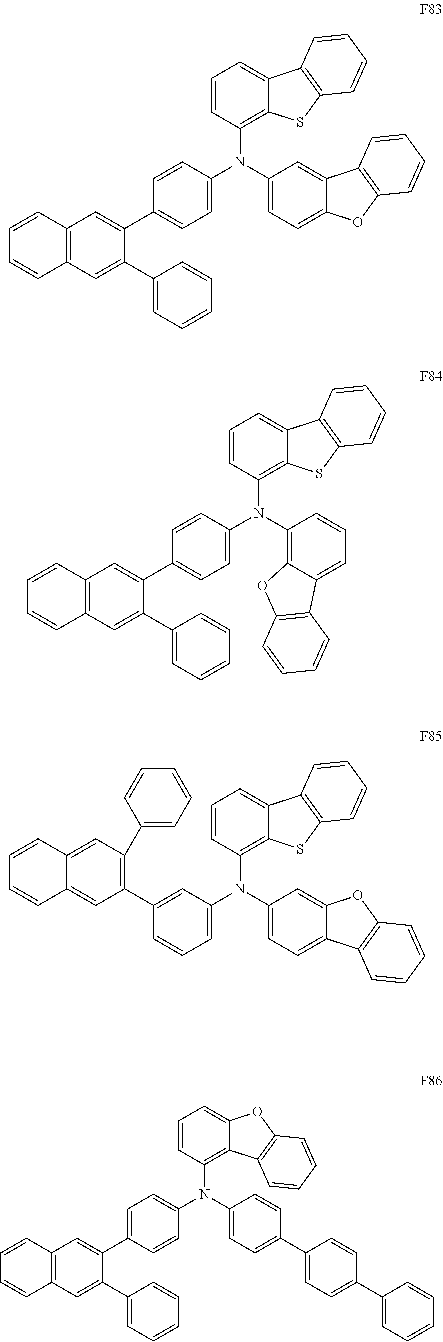 [SMILES: c1ccc(-c2cc3ccccc3cc2-c2ccc(N(c3ccc4oc5ccccc5c4c3)c3cccc4c3sc3ccccc34)cc2)cc1 ; c1ccc(-c2cc3ccccc3cc2-c2ccc(N(c3cccc4c3oc3ccccc34)c3cccc4c3sc3ccccc34)cc2)cc1 ; c1ccc(-c2cc3ccccc3cc2-c2cccc(N(c3ccc4c(c3)oc3ccccc34)c3cccc4c3sc3ccccc34)c2)cc1 ; c1ccc(-c2ccc(-c3ccc(N(c4ccc(-c5cc6ccccc6cc5-c5ccccc5)cc4)c4cccc5oc6ccccc6c45)cc3)cc2)cc1] 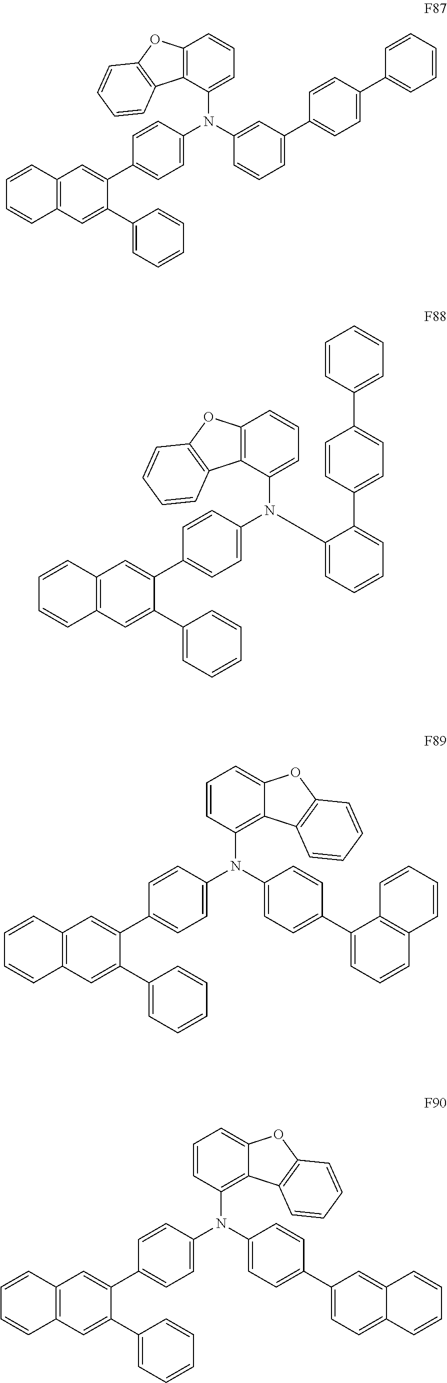 [SMILES: c1ccc(-c2ccc(-c3cccc(N(c4ccc(-c5cc6ccccc6cc5-c5ccccc5)cc4)c4cccc5oc6ccccc6c45)c3)cc2)cc1 ; c1ccc(-c2ccc(-c3ccccc3N(c3ccc(-c4cc5ccccc5cc4-c4ccccc4)cc3)c3cccc4oc5ccccc5c34)cc2)cc1 ; c1ccc(-c2cc3ccccc3cc2-c2ccc(N(c3ccc(-c4cccc5ccccc45)cc3)c3cccc4oc5ccccc5c34)cc2)cc1 ; c1ccc(-c2cc3ccccc3cc2-c2ccc(N(c3ccc(-c4ccc5ccccc5c4)cc3)c3cccc4oc5ccccc5c34)cc2)cc1] 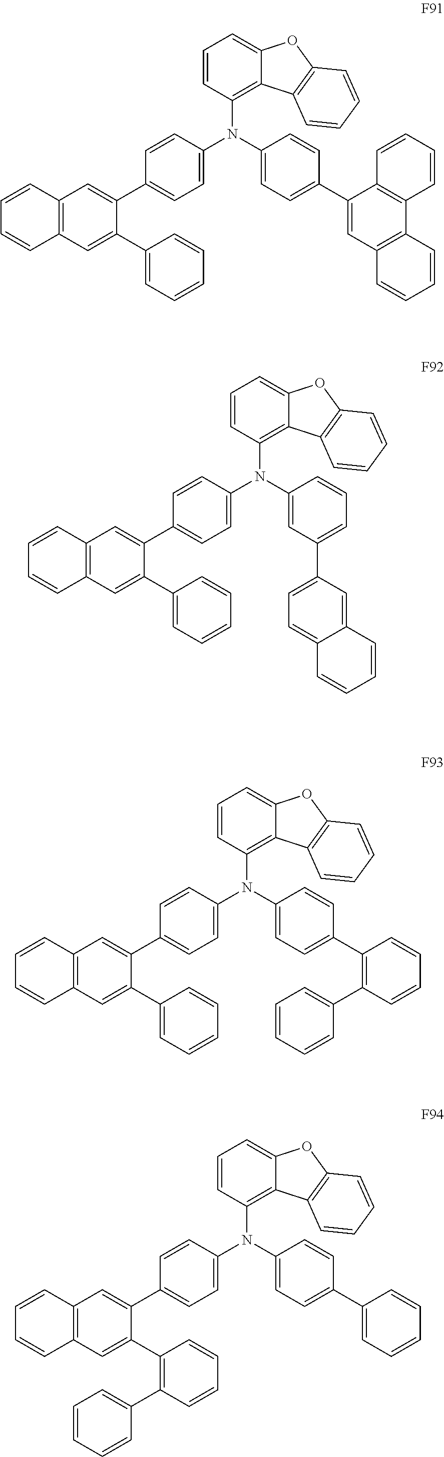 [SMILES: c1ccc(-c2cc3ccccc3cc2-c2ccc(N(c3ccc(-c4cc5ccccc5c5ccccc45)cc3)c3cccc4oc5ccccc5c34)cc2)cc1 ; c1ccc(-c2cc3ccccc3cc2-c2ccc(N(c3cccc(-c4ccc5ccccc5c4)c3)c3cccc4oc5ccccc5c34)cc2)cc1 ; c1ccc(-c2ccccc2-c2ccc(N(c3ccc(-c4cc5ccccc5cc4-c4ccccc4)cc3)c3cccc4oc5ccccc5c34)cc2)cc1 ; c1ccc(-c2ccc(N(c3ccc(-c4cc5ccccc5cc4-c4ccccc4-c4ccccc4)cc3)c3cccc4oc5ccccc5c34)cc2)cc1] 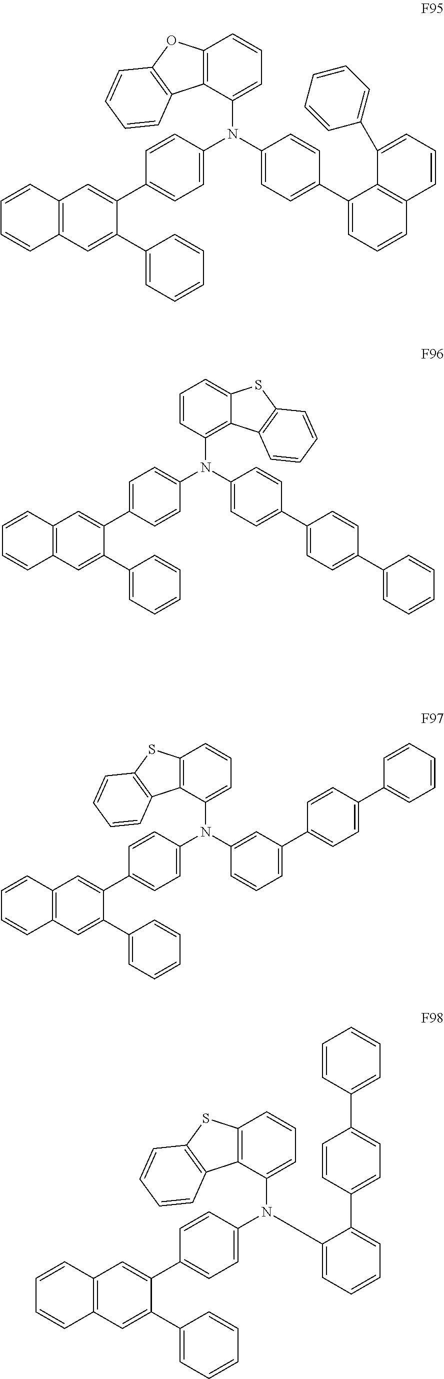 [SMILES: c1ccc(-c2cc3ccccc3cc2-c2ccc(N(c3ccc(-c4cccc5cccc(-c6ccccc6)c45)cc3)c3cccc4oc5ccccc5c34)cc2)cc1 ; c1ccc(-c2ccc(-c3ccc(N(c4ccc(-c5cc6ccccc6cc5-c5ccccc5)cc4)c4cccc5sc6ccccc6c45)cc3)cc2)cc1 ; c1ccc(-c2ccc(-c3cccc(N(c4ccc(-c5cc6ccccc6cc5-c5ccccc5)cc4)c4cccc5sc6ccccc6c45)c3)cc2)cc1 ; c1ccc(-c2ccc(-c3ccccc3N(c3ccc(-c4cc5ccccc5cc4-c4ccccc4)cc3)c3cccc4sc5ccccc5c34)cc2)cc1] 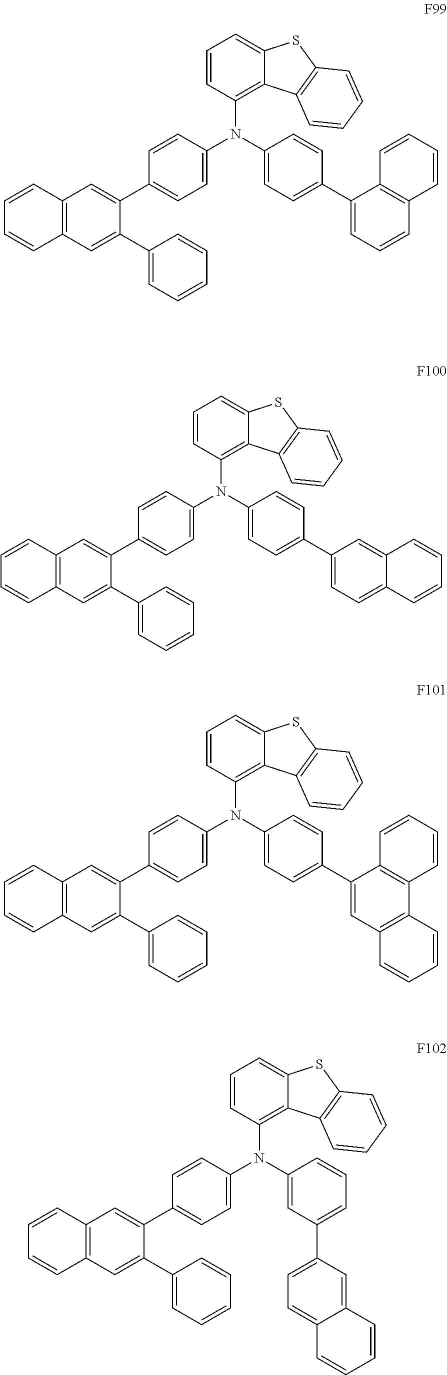 [SMILES: c1ccc(-c2cc3ccccc3cc2-c2ccc(N(c3ccc(-c4cccc5ccccc45)cc3)c3cccc4sc5ccccc5c34)cc2)cc1 ; c1ccc(-c2cc3ccccc3cc2-c2ccc(N(c3ccc(-c4ccc5ccccc5c4)cc3)c3cccc4sc5ccccc5c34)cc2)cc1 ; c1ccc(-c2cc3ccccc3cc2-c2ccc(N(c3ccc(-c4cc5ccccc5c5ccccc45)cc3)c3cccc4sc5ccccc5c34)cc2)cc1 ; c1ccc(-c2cc3ccccc3cc2-c2ccc(N(c3cccc(-c4ccc5ccccc5c4)c3)c3cccc4sc5ccccc5c34)cc2)cc1] 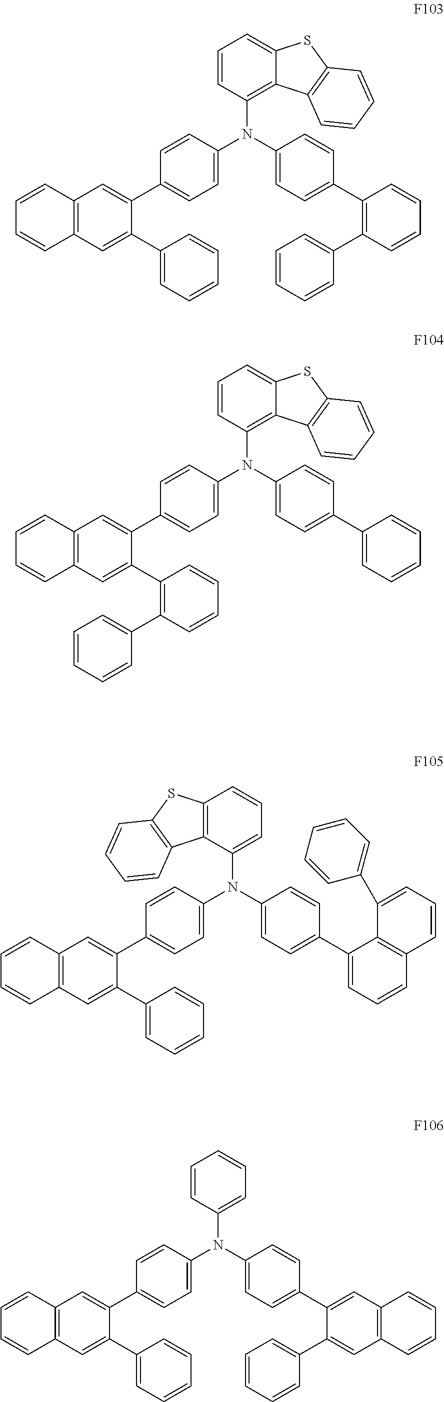 [SMILES: c1ccc(-c2ccccc2-c2ccc(N(c3ccc(-c4cc5ccccc5cc4-c4ccccc4)cc3)c3cccc4sc5ccccc5c34)cc2)cc1 ; c1ccc(-c2ccc(N(c3ccc(-c4cc5ccccc5cc4-c4ccccc4-c4ccccc4)cc3)c3cccc4sc5ccccc5c34)cc2)cc1 ; c1ccc(-c2cc3ccccc3cc2-c2ccc(N(c3ccc(-c4cccc5cccc(-c6ccccc6)c45)cc3)c3cccc4sc5ccccc5c34)cc2)cc1 ; c1ccc(-c2cc3ccccc3cc2-c2ccc(N(c3ccccc3)c3ccc(-c4cc5ccccc5cc4-c4ccccc4)cc3)cc2)cc1] 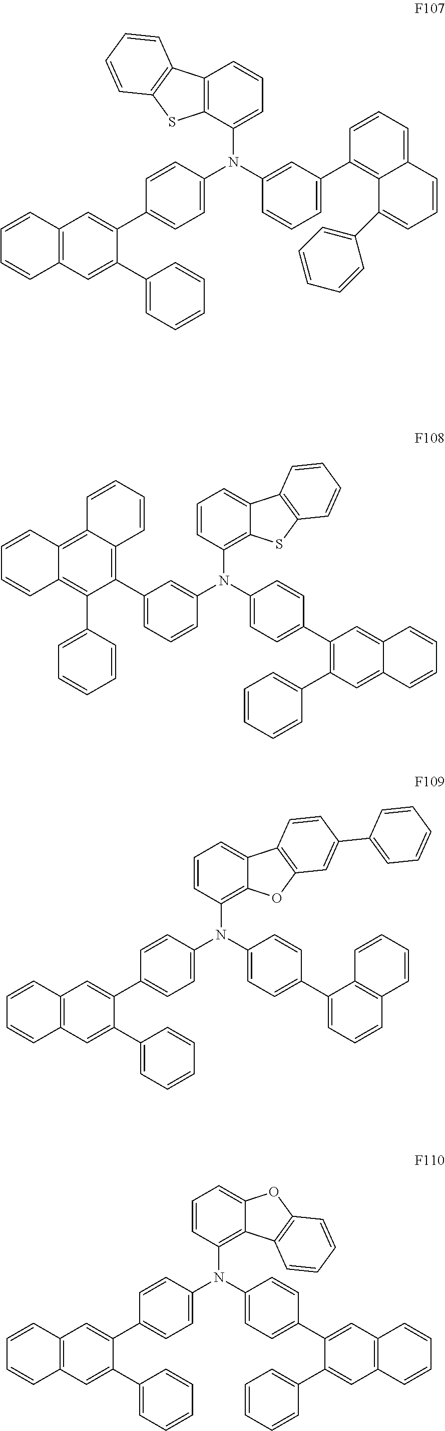 [SMILES: c1ccc(-c2cc3ccccc3cc2-c2ccc(N(c3cccc(-c4cccc5cccc(-c6ccccc6)c45)c3)c3cccc4c3sc3ccccc34)cc2)cc1 ; c1ccc(-c2cc3ccccc3cc2-c2ccc(N(c3cccc(-c4c(-c5ccccc5)c5ccccc5c5ccccc45)c3)c3cccc4c3sc3ccccc34)cc2)cc1 ; c1ccc(-c2ccc3c(c2)oc2c(N(c4ccc(-c5cc6ccccc6cc5-c5ccccc5)cc4)c4ccc(-c5cccc6ccccc56)cc4)cccc23)cc1 ; c1ccc(-c2cc3ccccc3cc2-c2ccc(N(c3ccc(-c4cc5ccccc5cc4-c4ccccc4)cc3)c3cccc4oc5ccccc5c34)cc2)cc1] 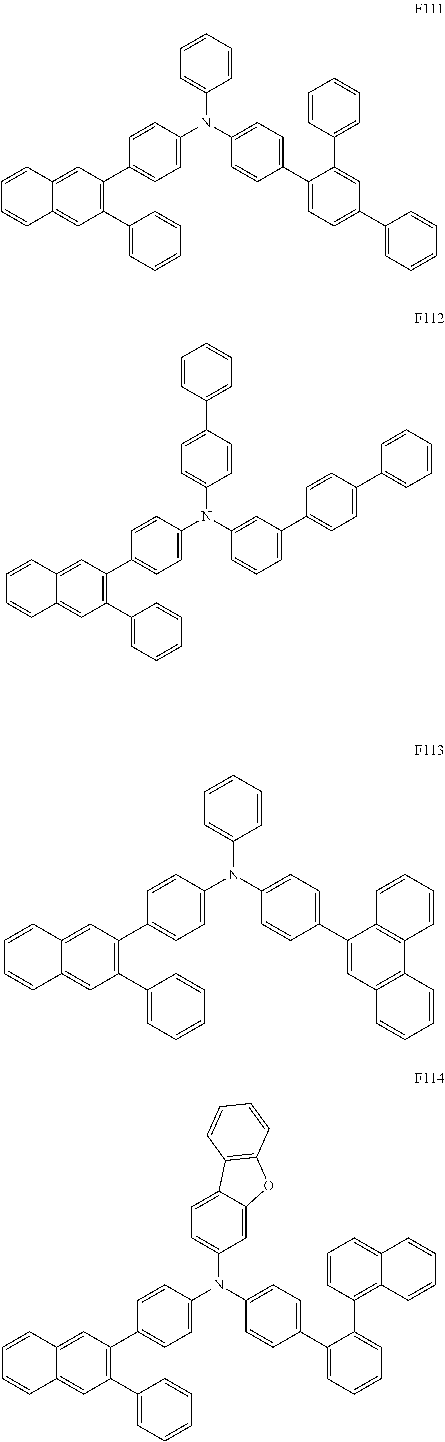 [SMILES: c1ccc(-c2ccc(-c3ccc(N(c4ccccc4)c4ccc(-c5cc6ccccc6cc5-c5ccccc5)cc4)cc3)c(-c3ccccc3)c2)cc1 ; c1ccc(-c2ccc(-c3cccc(N(c4ccc(-c5ccccc5)cc4)c4ccc(-c5cc6ccccc6cc5-c5ccccc5)cc4)c3)cc2)cc1 ; c1ccc(-c2cc3ccccc3cc2-c2ccc(N(c3ccccc3)c3ccc(-c4cc5ccccc5c5ccccc45)cc3)cc2)cc1 ; c1ccc(-c2cc3ccccc3cc2-c2ccc(N(c3ccc(-c4ccccc4-c4cccc5ccccc45)cc3)c3ccc4c(c3)oc3ccccc34)cc2)cc1] 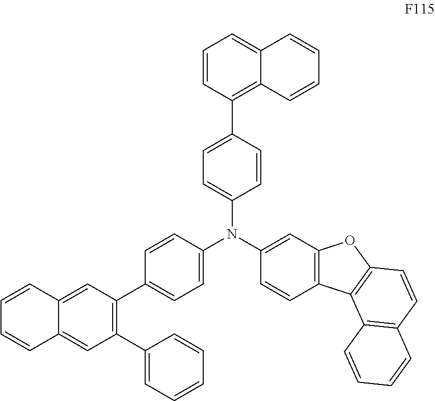 [SMILES: c1ccc(-c2cc3ccccc3cc2-c2ccc(N(c3ccc(-c4cccc5ccccc45)cc3)c3ccc4c(c3)oc3ccc5ccccc5c34)cc2)cc1] 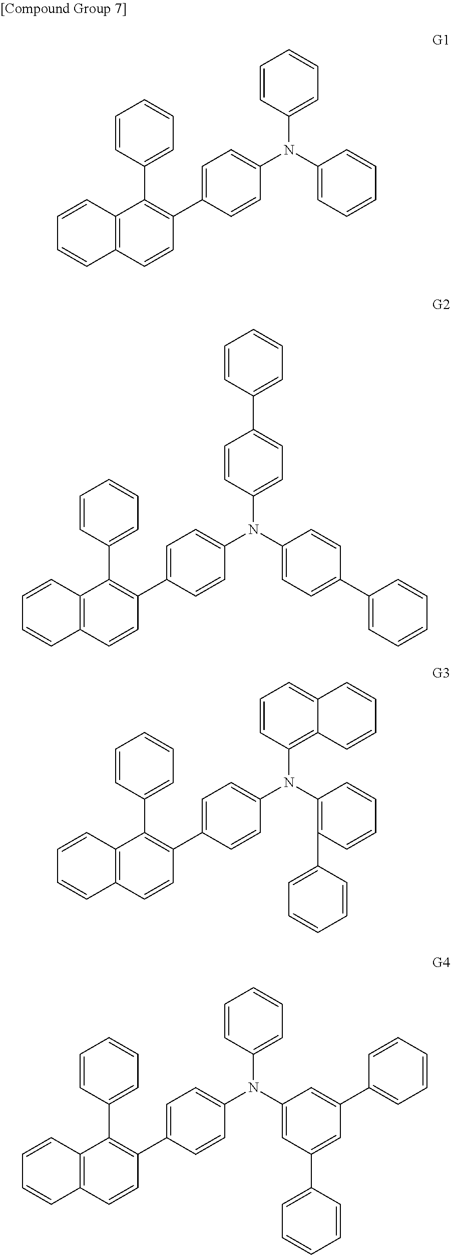 [SMILES: c1ccc(-c2c(-c3ccc(N(c4ccccc4)c4ccccc4)cc3)ccc3ccccc23)cc1 ; c1ccc(-c2ccc(N(c3ccc(-c4ccccc4)cc3)c3ccc(-c4ccc5ccccc5c4-c4ccccc4)cc3)cc2)cc1 ; c1ccc(-c2ccccc2N(c2ccc(-c3ccc4ccccc4c3-c3ccccc3)cc2)c2cccc3ccccc23)cc1 ; c1ccc(-c2cc(-c3ccccc3)cc(N(c3ccccc3)c3ccc(-c4ccc5ccccc5c4-c4ccccc4)cc3)c2)cc1] 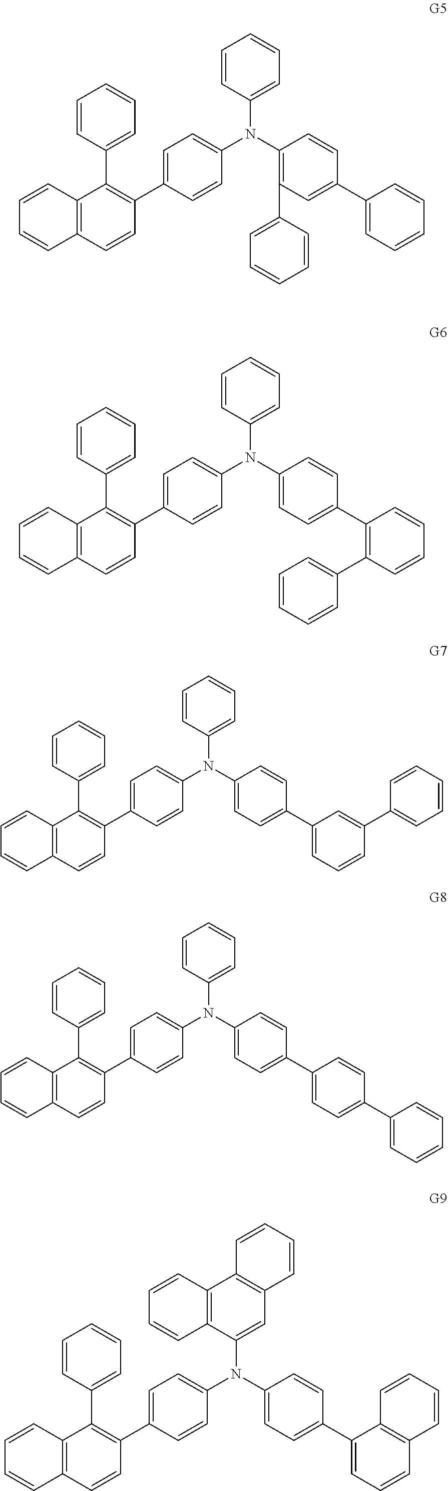 [SMILES: c1ccc(-c2ccc(N(c3ccccc3)c3ccc(-c4ccc5ccccc5c4-c4ccccc4)cc3)c(-c3ccccc3)c2)cc1 ; c1ccc(-c2ccccc2-c2ccc(N(c3ccccc3)c3ccc(-c4ccc5ccccc5c4-c4ccccc4)cc3)cc2)cc1 ; c1ccc(-c2cccc(-c3ccc(N(c4ccccc4)c4ccc(-c5ccc6ccccc6c5-c5ccccc5)cc4)cc3)c2)cc1 ; c1ccc(-c2ccc(-c3ccc(N(c4ccccc4)c4ccc(-c5ccc6ccccc6c5-c5ccccc5)cc4)cc3)cc2)cc1 ; c1ccc(-c2c(-c3ccc(N(c4ccc(-c5cccc6ccccc56)cc4)c4cc5ccccc5c5ccccc45)cc3)ccc3ccccc23)cc1] 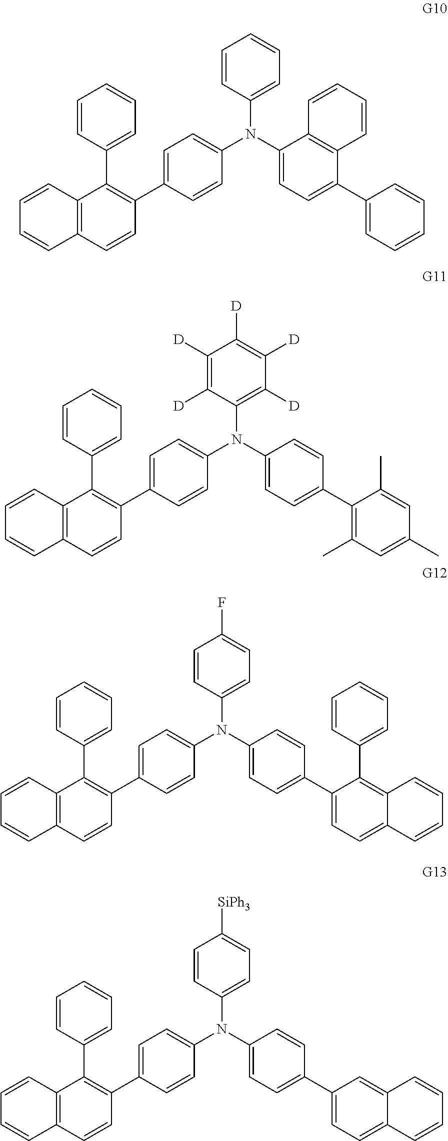 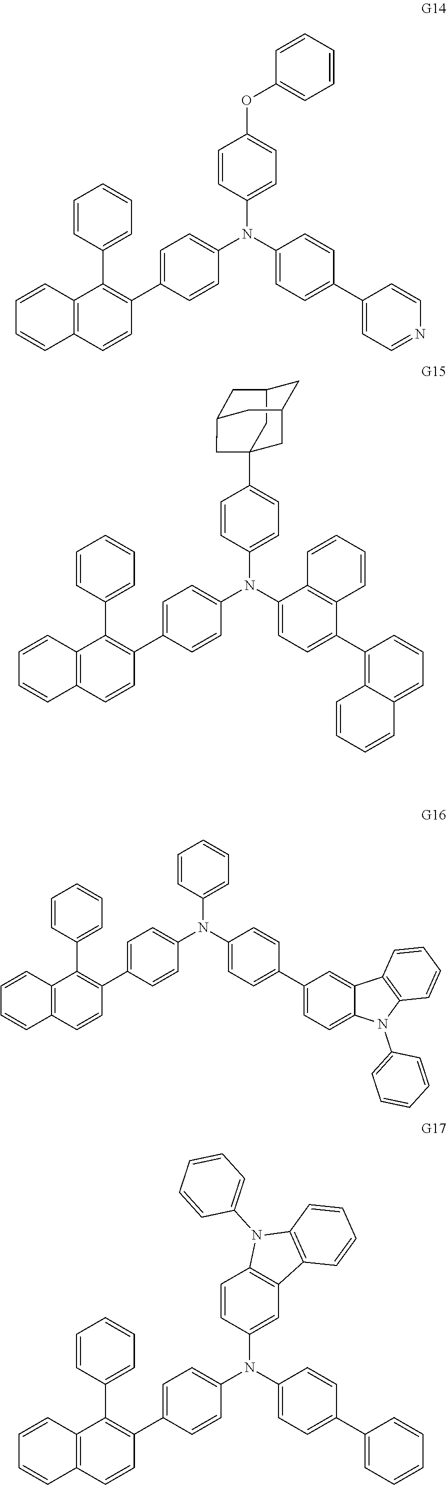 [SMILES: c1ccc(Oc2ccc(N(c3ccc(-c4ccncc4)cc3)c3ccc(-c4ccc5ccccc5c4-c4ccccc4)cc3)cc2)cc1 ; c1ccc(-c2c(-c3ccc(N(c4ccc(C56CC7CC(CC(C7)C5)C6)cc4)c4ccc(-c5cccc6ccccc56)c5ccccc45)cc3)ccc3ccccc23)cc1 ; c1ccc(-c2c(-c3ccc(N(c4ccccc4)c4ccc(-c5ccc6c(c5)c5ccccc5n6-c5ccccc5)cc4)cc3)ccc3ccccc23)cc1 ; c1ccc(-c2ccc(N(c3ccc(-c4ccc5ccccc5c4-c4ccccc4)cc3)c3ccc4c(c3)c3ccccc3n4-c3ccccc3)cc2)cc1] 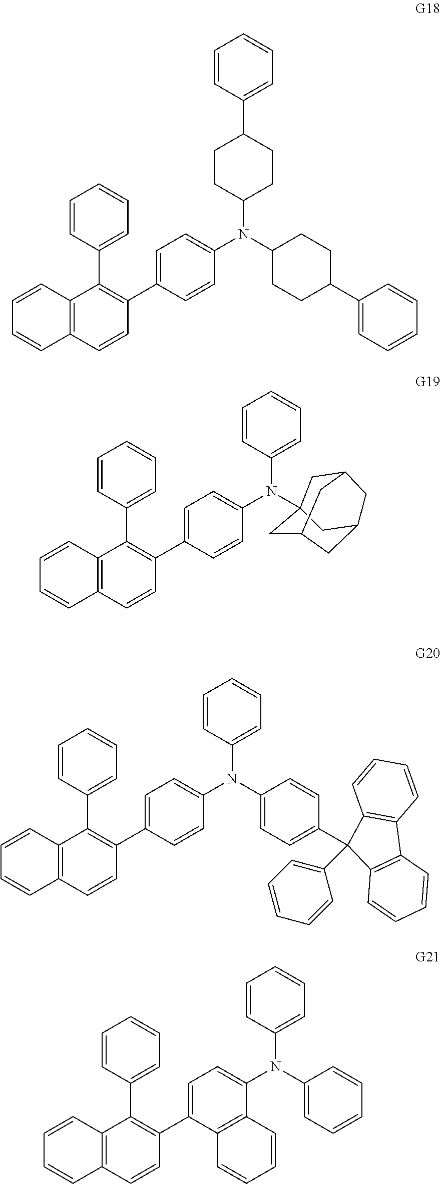 [SMILES: c1ccc(-c2c(-c3ccc(N(C4CCC(c5ccccc5)CC4)C4CCC(c5ccccc5)CC4)cc3)ccc3ccccc23)cc1 ; c1ccc(-c2c(-c3ccc(N(c4ccccc4)C45CC6CC(CC(C6)C4)C5)cc3)ccc3ccccc23)cc1 ; c1ccc(-c2c(-c3ccc(N(c4ccccc4)c4ccc(C5(c6ccccc6)c6ccccc6-c6ccccc65)cc4)cc3)ccc3ccccc23)cc1 ; c1ccc(-c2c(-c3ccc(N(c4ccccc4)c4ccccc4)c4ccccc34)ccc3ccccc23)cc1] 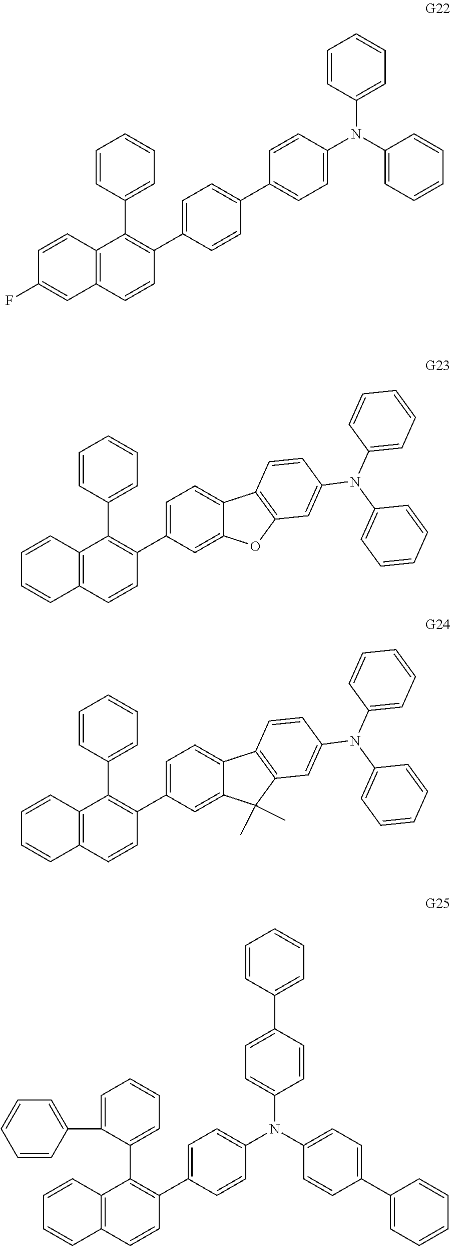 [SMILES: Fc1ccc2c(-c3ccccc3)c(-c3ccc(-c4ccc(N(c5ccccc5)c5ccccc5)cc4)cc3)ccc2c1 ; c1ccc(-c2c(-c3ccc4c(c3)oc3cc(N(c5ccccc5)c5ccccc5)ccc34)ccc3ccccc23)cc1 ; CC1(C)c2cc(-c3ccc4ccccc4c3-c3ccccc3)ccc2-c2ccc(N(c3ccccc3)c3ccccc3)cc21 ; c1ccc(-c2ccc(N(c3ccc(-c4ccccc4)cc3)c3ccc(-c4ccc5ccccc5c4-c4ccccc4-c4ccccc4)cc3)cc2)cc1] 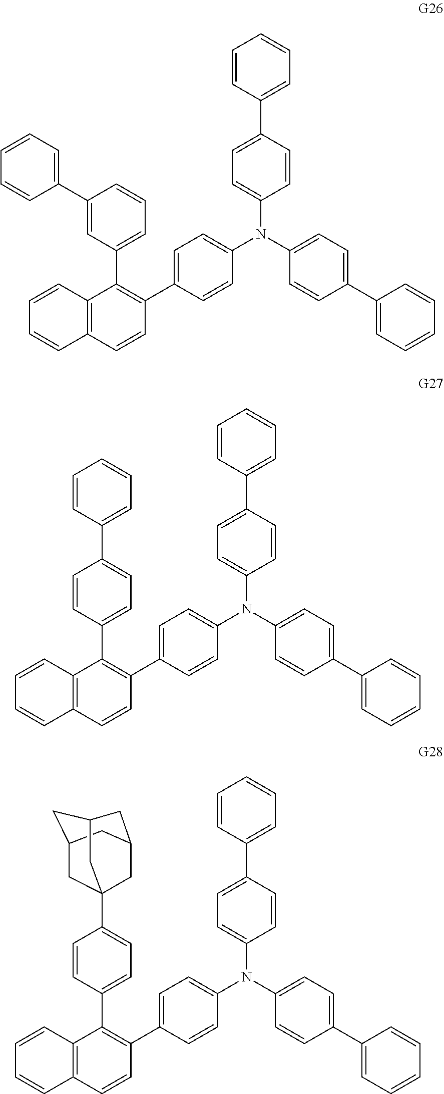 [SMILES: c1ccc(-c2ccc(N(c3ccc(-c4ccccc4)cc3)c3ccc(-c4ccc5ccccc5c4-c4cccc(-c5ccccc5)c4)cc3)cc2)cc1 ; c1ccc(-c2ccc(-c3c(-c4ccc(N(c5ccc(-c6ccccc6)cc5)c5ccc(-c6ccccc6)cc5)cc4)ccc4ccccc34)cc2)cc1 ; c1ccc(-c2ccc(N(c3ccc(-c4ccccc4)cc3)c3ccc(-c4ccc5ccccc5c4-c4ccc(C56CC7CC(CC(C7)C5)C6)cc4)cc3)cc2)cc1] 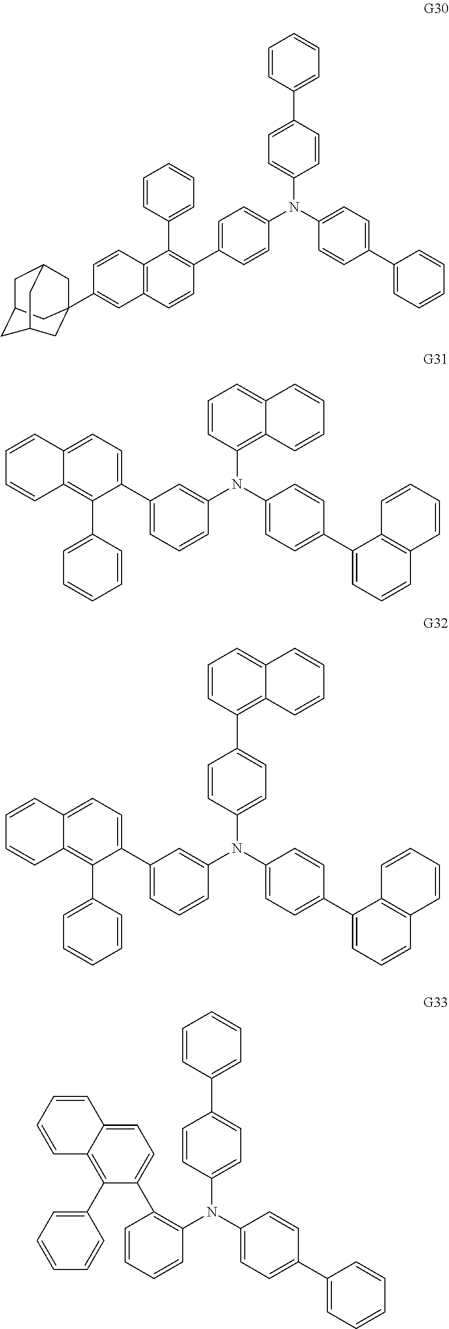 [SMILES: c1ccc(-c2ccc(N(c3ccc(-c4ccccc4)cc3)c3ccc(-c4ccc5cc(C67CC8CC(CC(C8)C6)C7)ccc5c4-c4ccccc4)cc3)cc2)cc1 ; c1ccc(-c2c(-c3cccc(N(c4ccc(-c5cccc6ccccc56)cc4)c4cccc5ccccc45)c3)ccc3ccccc23)cc1 ; c1ccc(-c2c(-c3cccc(N(c4ccc(-c5cccc6ccccc56)cc4)c4ccc(-c5cccc6ccccc56)cc4)c3)ccc3ccccc23)cc1 ; c1ccc(-c2ccc(N(c3ccc(-c4ccccc4)cc3)c3ccccc3-c3ccc4ccccc4c3-c3ccccc3)cc2)cc1] 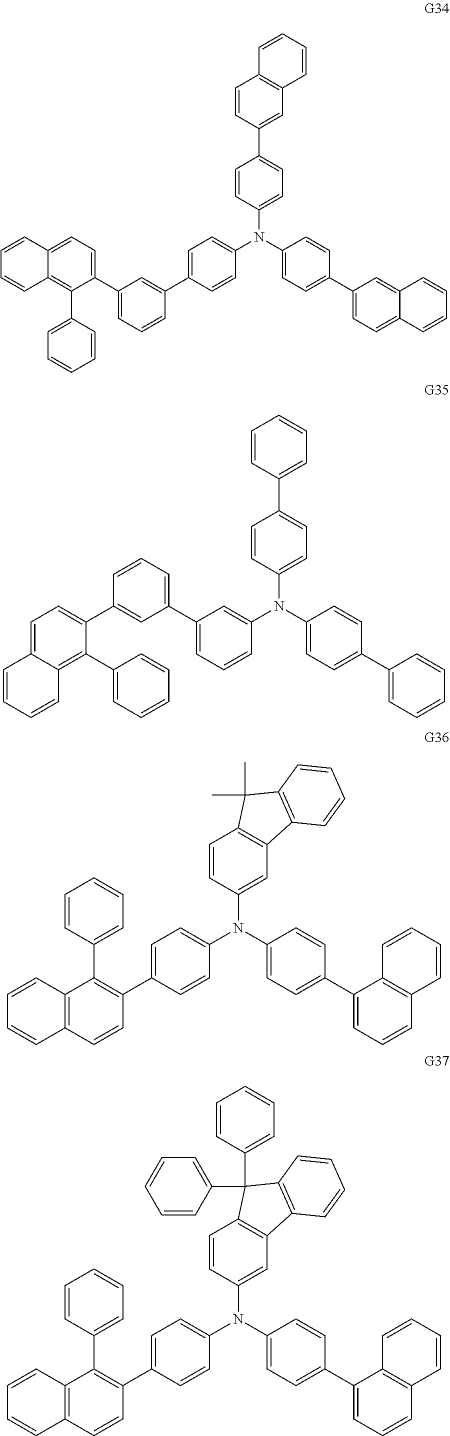 [SMILES: c1ccc(-c2c(-c3cccc(-c4ccc(N(c5ccc(-c6ccc7ccccc7c6)cc5)c5ccc(-c6ccc7ccccc7c6)cc5)cc4)c3)ccc3ccccc23)cc1 ; c1ccc(-c2ccc(N(c3ccc(-c4ccccc4)cc3)c3cccc(-c4cccc(-c5ccc6ccccc6c5-c5ccccc5)c4)c3)cc2)cc1 ; CC1(C)c2ccccc2-c2cc(N(c3ccc(-c4ccc5ccccc5c4-c4ccccc4)cc3)c3ccc(-c4cccc5ccccc45)cc3)ccc21 ; c1ccc(-c2c(-c3ccc(N(c4ccc(-c5cccc6ccccc56)cc4)c4ccc5c(c4)-c4ccccc4C5(c4ccccc4)c4ccccc4)cc3)ccc3ccccc23)cc1] 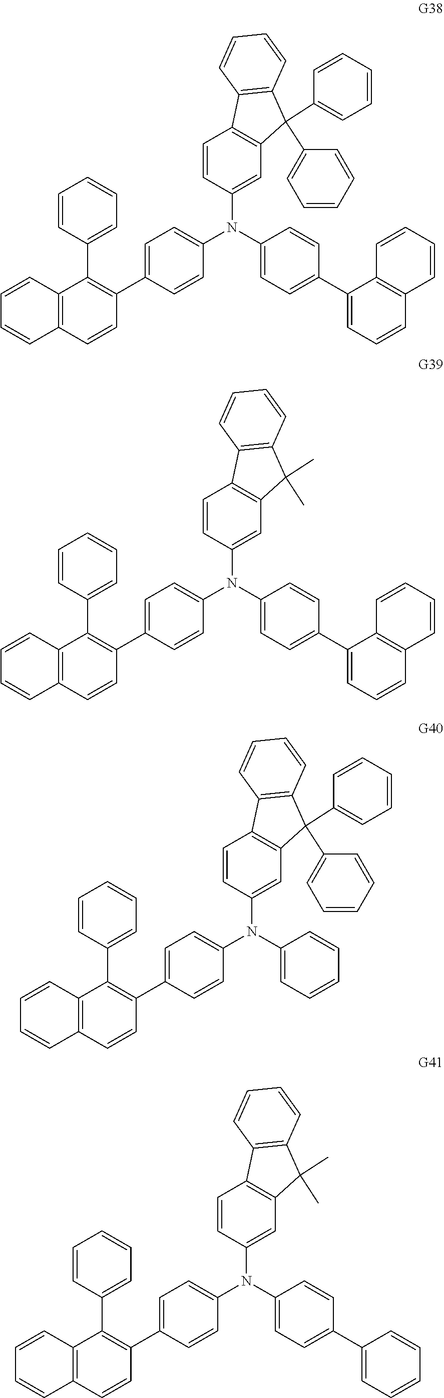 [SMILES: c1ccc(-c2c(-c3ccc(N(c4ccc(-c5cccc6ccccc56)cc4)c4ccc5c(c4)C(c4ccccc4)(c4ccccc4)c4ccccc4-5)cc3)ccc3ccccc23)cc1 ; CC1(C)c2ccccc2-c2ccc(N(c3ccc(-c4ccc5ccccc5c4-c4ccccc4)cc3)c3ccc(-c4cccc5ccccc45)cc3)cc21 ; c1ccc(-c2c(-c3ccc(N(c4ccccc4)c4ccc5c(c4)C(c4ccccc4)(c4ccccc4)c4ccccc4-5)cc3)ccc3ccccc23)cc1 ; CC1(C)c2ccccc2-c2ccc(N(c3ccc(-c4ccccc4)cc3)c3ccc(-c4ccc5ccccc5c4-c4ccccc4)cc3)cc21] 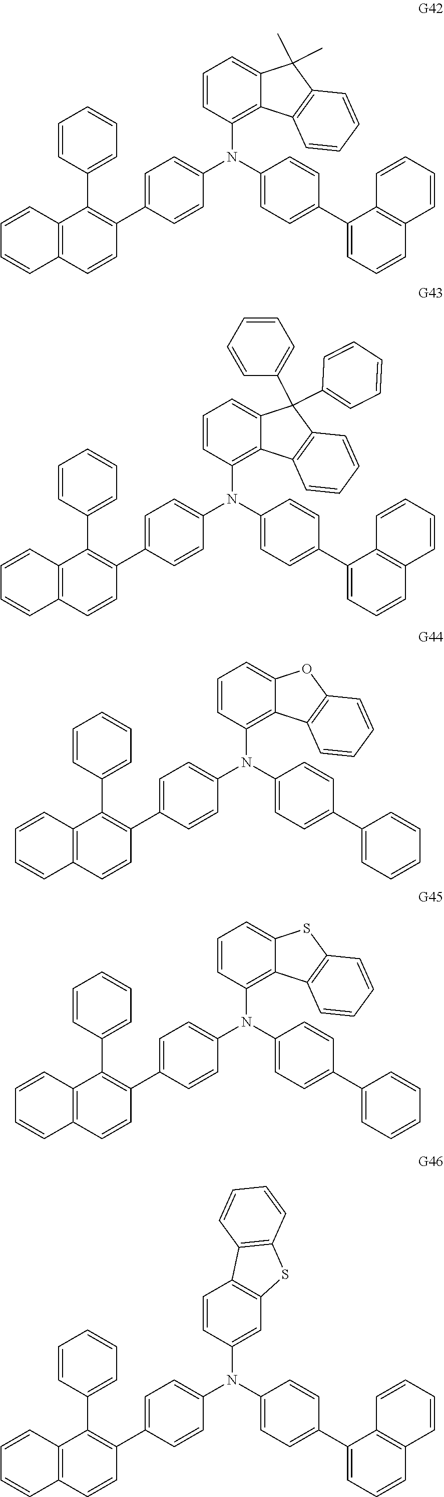 [SMILES: CC1(C)c2ccccc2-c2c(N(c3ccc(-c4ccc5ccccc5c4-c4ccccc4)cc3)c3ccc(-c4cccc5ccccc45)cc3)cccc21 ; c1ccc(-c2c(-c3ccc(N(c4ccc(-c5cccc6ccccc56)cc4)c4cccc5c4-c4ccccc4C5(c4ccccc4)c4ccccc4)cc3)ccc3ccccc23)cc1 ; c1ccc(-c2ccc(N(c3ccc(-c4ccc5ccccc5c4-c4ccccc4)cc3)c3cccc4oc5ccccc5c34)cc2)cc1 ; c1ccc(-c2ccc(N(c3ccc(-c4ccc5ccccc5c4-c4ccccc4)cc3)c3cccc4sc5ccccc5c34)cc2)cc1 ; c1ccc(-c2c(-c3ccc(N(c4ccc(-c5cccc6ccccc56)cc4)c4ccc5c(c4)sc4ccccc45)cc3)ccc3ccccc23)cc1] 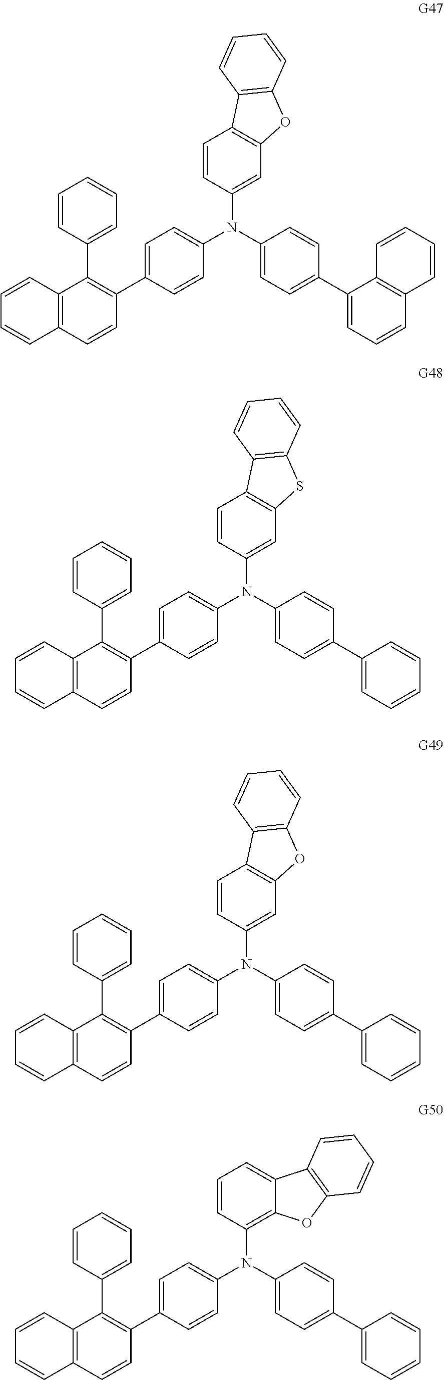 [SMILES: c1ccc(-c2c(-c3ccc(N(c4ccc(-c5cccc6ccccc56)cc4)c4ccc5c(c4)oc4ccccc45)cc3)ccc3ccccc23)cc1 ; c1ccc(-c2ccc(N(c3ccc(-c4ccc5ccccc5c4-c4ccccc4)cc3)c3ccc4c(c3)sc3ccccc34)cc2)cc1 ; c1ccc(-c2ccc(N(c3ccc(-c4ccc5ccccc5c4-c4ccccc4)cc3)c3ccc4c(c3)oc3ccccc34)cc2)cc1 ; c1ccc(-c2ccc(N(c3ccc(-c4ccc5ccccc5c4-c4ccccc4)cc3)c3cccc4c3oc3ccccc34)cc2)cc1] 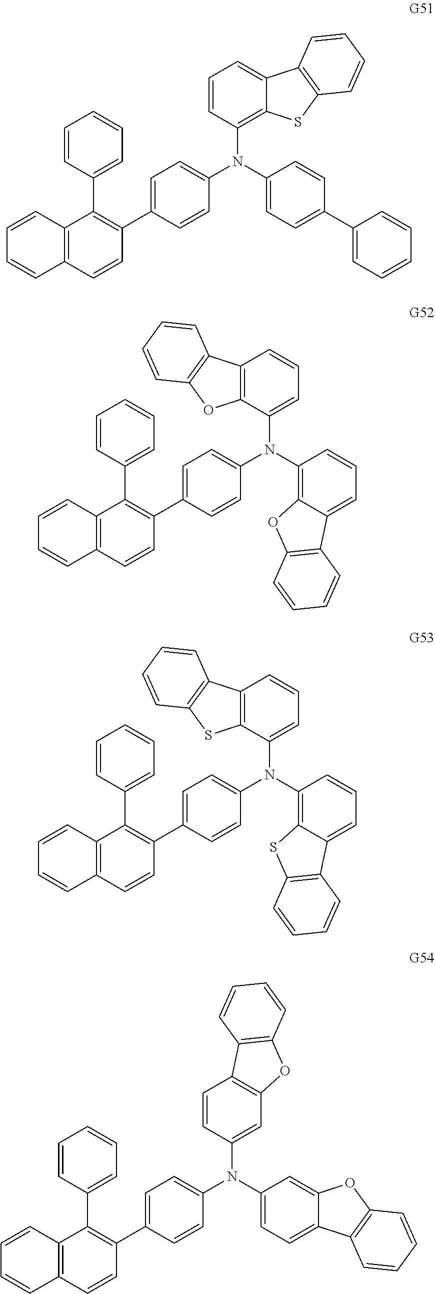 [SMILES: c1ccc(-c2ccc(N(c3ccc(-c4ccc5ccccc5c4-c4ccccc4)cc3)c3cccc4c3sc3ccccc34)cc2)cc1 ; c1ccc(-c2c(-c3ccc(N(c4cccc5c4oc4ccccc45)c4cccc5c4oc4ccccc45)cc3)ccc3ccccc23)cc1 ; c1ccc(-c2c(-c3ccc(N(c4cccc5c4sc4ccccc45)c4cccc5c4sc4ccccc45)cc3)ccc3ccccc23)cc1 ; c1ccc(-c2c(-c3ccc(N(c4ccc5c(c4)oc4ccccc45)c4ccc5c(c4)oc4ccccc45)cc3)ccc3ccccc23)cc1] 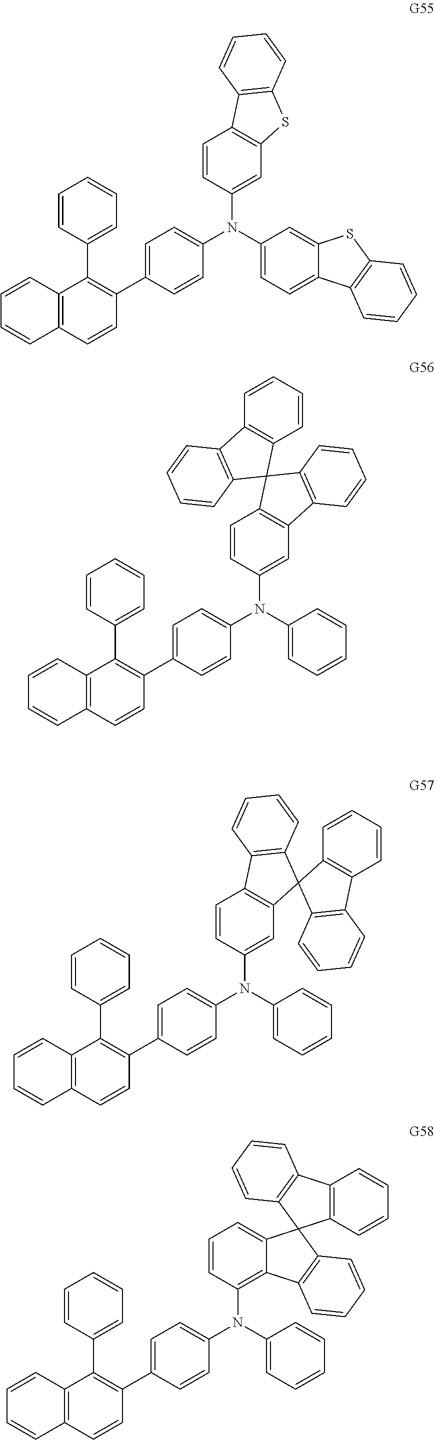 [SMILES: c1ccc(-c2c(-c3ccc(N(c4ccc5c(c4)sc4ccccc45)c4ccc5c(c4)sc4ccccc45)cc3)ccc3ccccc23)cc1 ; c1ccc(-c2c(-c3ccc(N(c4ccccc4)c4ccc5c(c4)-c4ccccc4C54c5ccccc5-c5ccccc54)cc3)ccc3ccccc23)cc1 ; c1ccc(-c2c(-c3ccc(N(c4ccccc4)c4ccc5c(c4)C4(c6ccccc6-c6ccccc64)c4ccccc4-5)cc3)ccc3ccccc23)cc1 ; c1ccc(-c2c(-c3ccc(N(c4ccccc4)c4cccc5c4-c4ccccc4C54c5ccccc5-c5ccccc54)cc3)ccc3ccccc23)cc1] 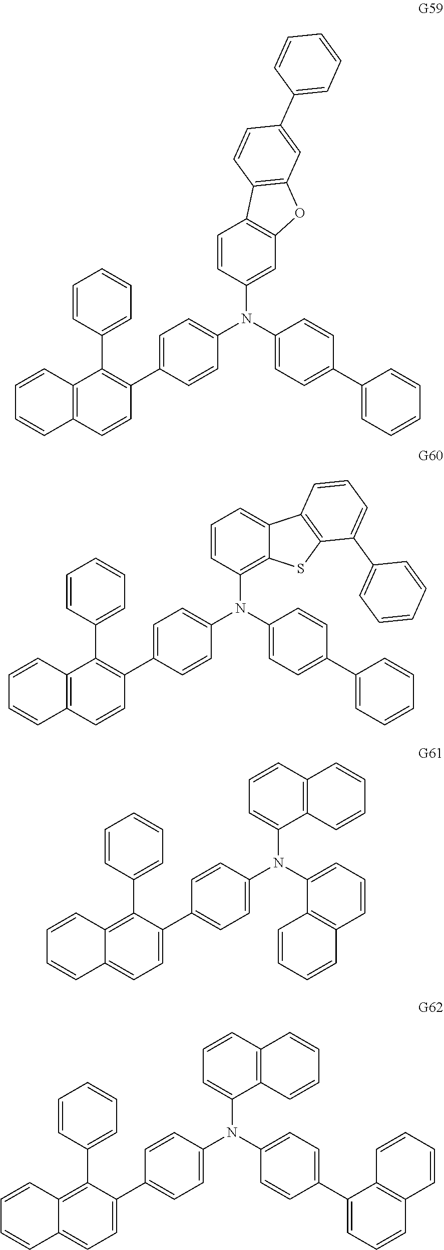 [SMILES: c1ccc(-c2ccc(N(c3ccc(-c4ccc5ccccc5c4-c4ccccc4)cc3)c3ccc4c(c3)oc3cc(-c5ccccc5)ccc34)cc2)cc1 ; c1ccc(-c2ccc(N(c3ccc(-c4ccc5ccccc5c4-c4ccccc4)cc3)c3cccc4c3sc3c(-c5ccccc5)cccc34)cc2)cc1 ; c1ccc(-c2c(-c3ccc(N(c4cccc5ccccc45)c4cccc5ccccc45)cc3)ccc3ccccc23)cc1 ; c1ccc(-c2c(-c3ccc(N(c4ccc(-c5cccc6ccccc56)cc4)c4cccc5ccccc45)cc3)ccc3ccccc23)cc1] 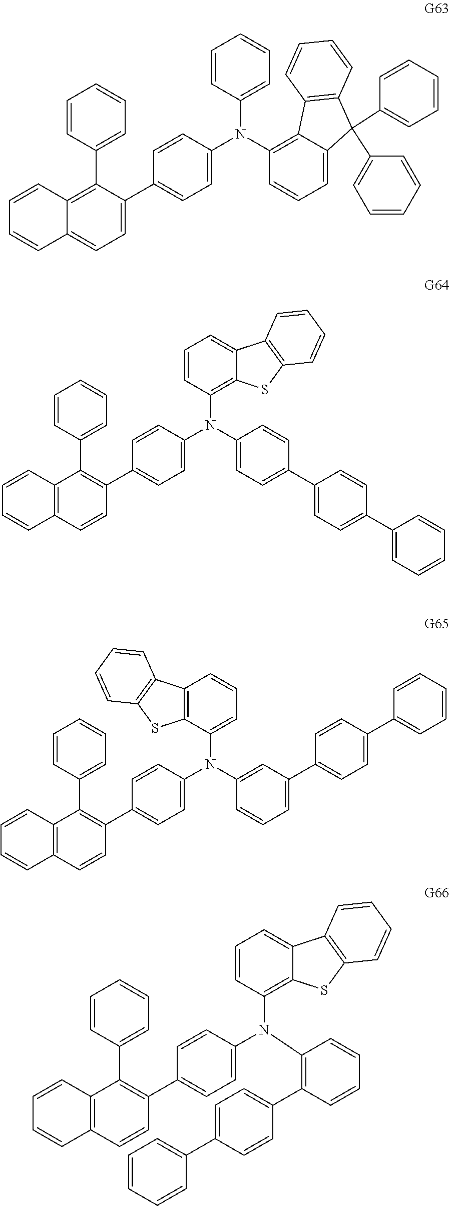 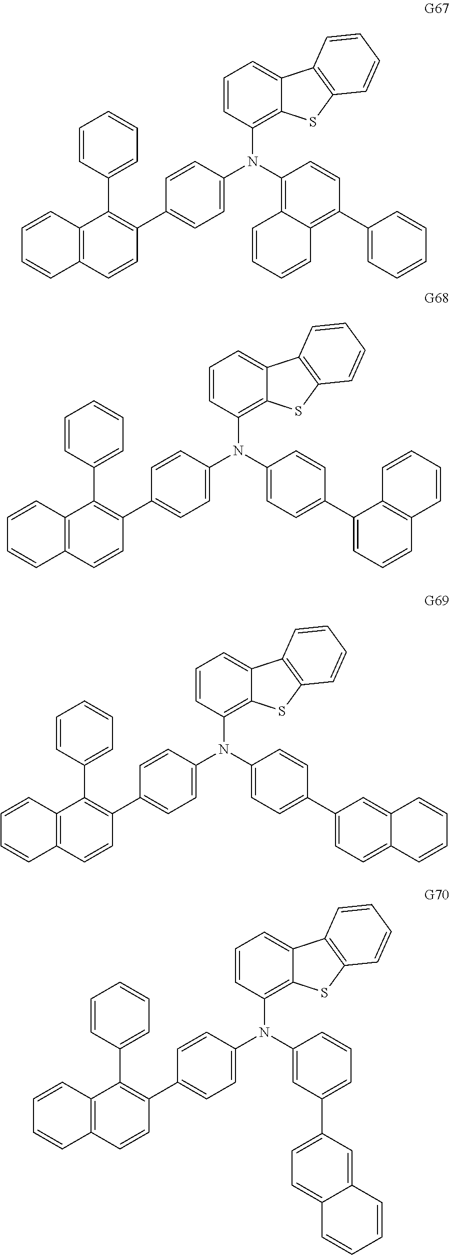 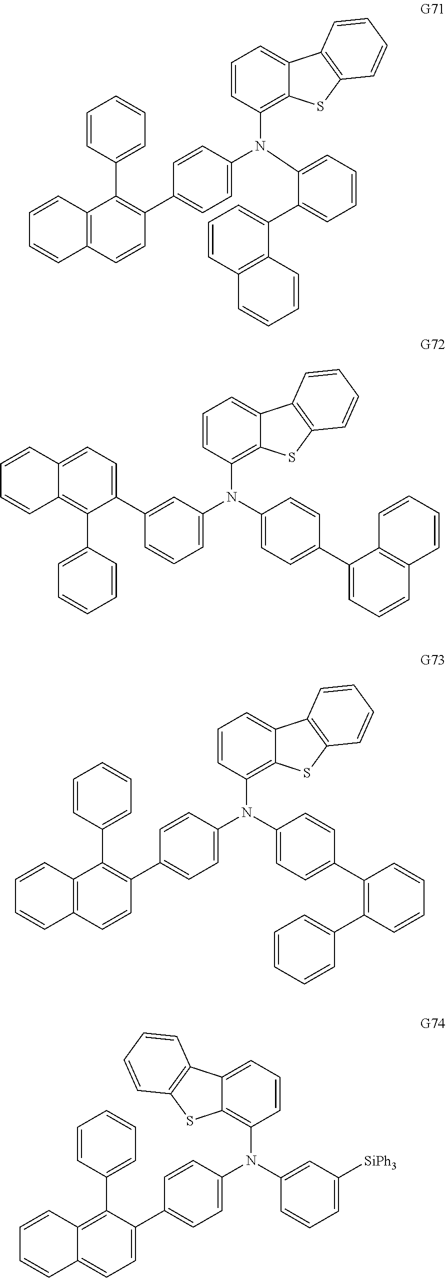 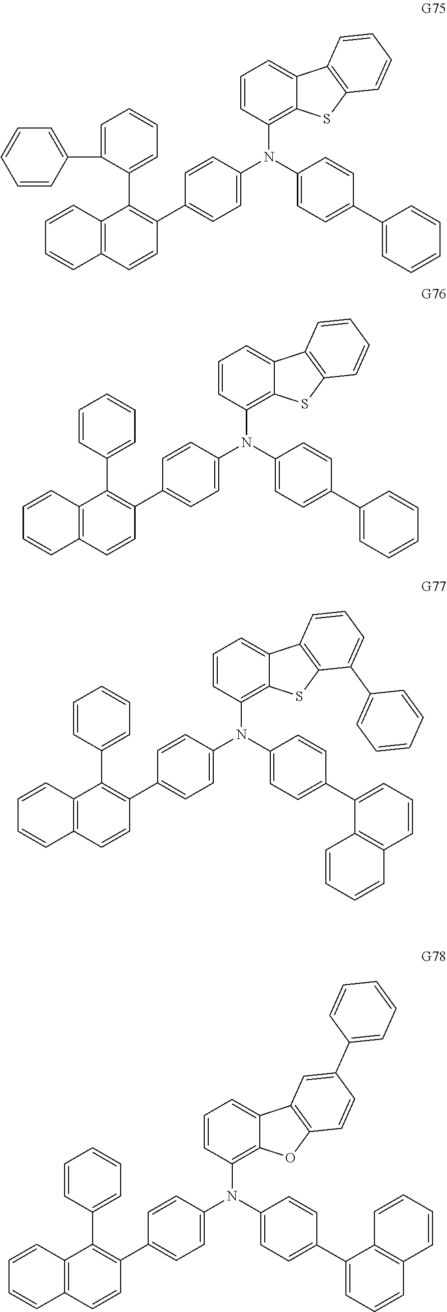 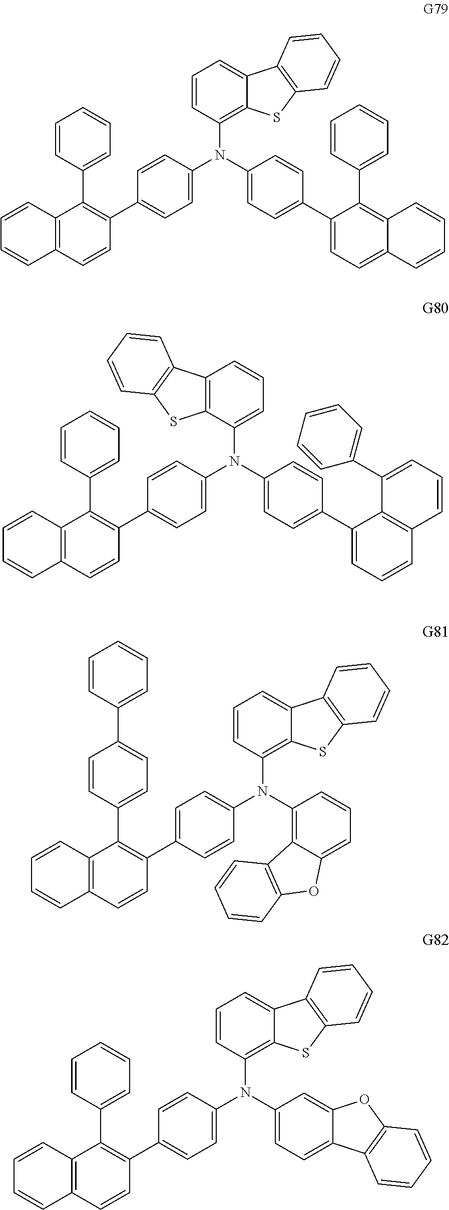 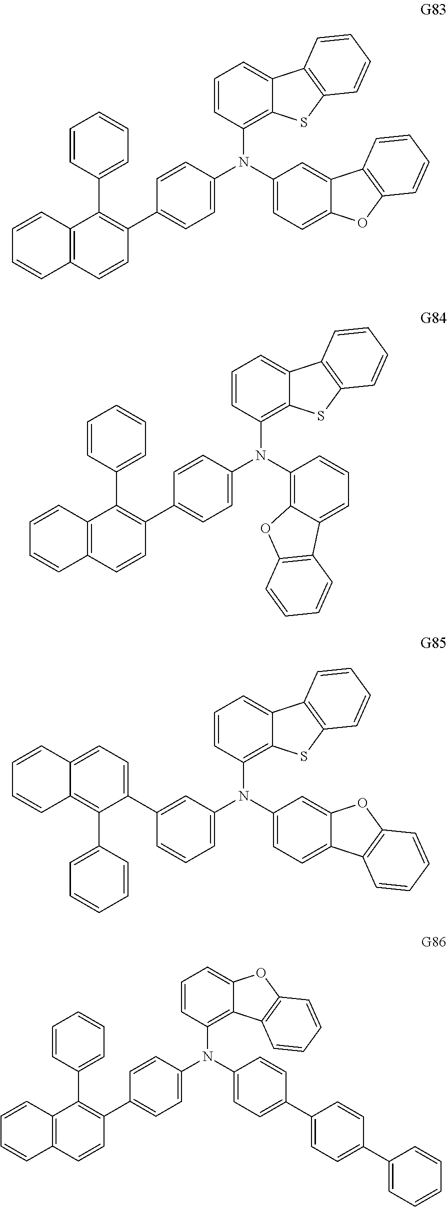 [SMILES: c1ccc(-c2c(-c3ccc(N(c4ccc5oc6ccccc6c5c4)c4cccc5c4sc4ccccc45)cc3)ccc3ccccc23)cc1 ; c1ccc(-c2c(-c3ccc(N(c4cccc5c4oc4ccccc45)c4cccc5c4sc4ccccc45)cc3)ccc3ccccc23)cc1 ; c1ccc(-c2c(-c3cccc(N(c4ccc5c(c4)oc4ccccc45)c4cccc5c4sc4ccccc45)c3)ccc3ccccc23)cc1 ; c1ccc(-c2ccc(-c3ccc(N(c4ccc(-c5ccc6ccccc6c5-c5ccccc5)cc4)c4cccc5oc6ccccc6c45)cc3)cc2)cc1] 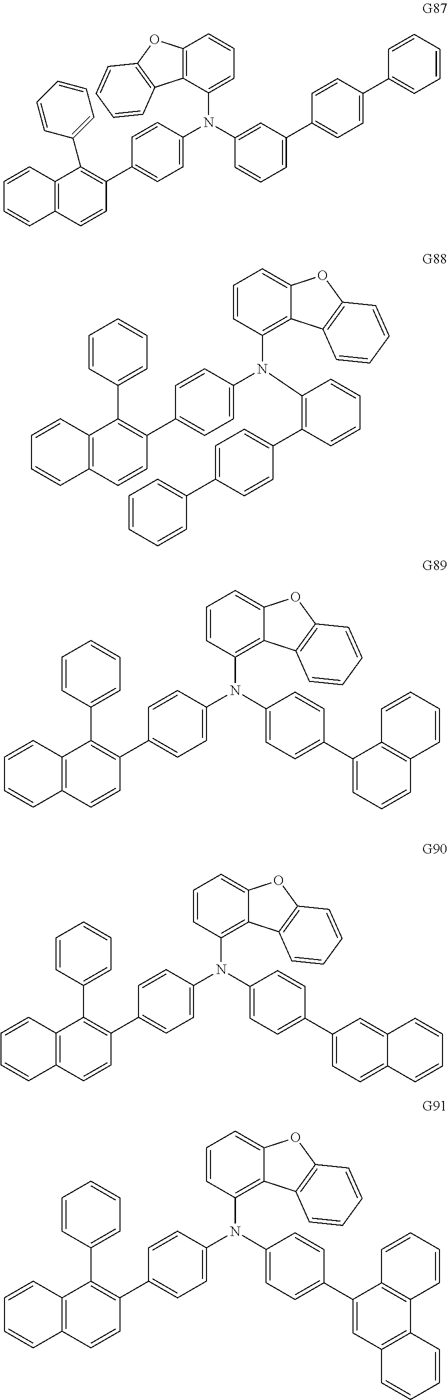 [SMILES: c1ccc(-c2ccc(-c3cccc(N(c4ccc(-c5ccc6ccccc6c5-c5ccccc5)cc4)c4cccc5oc6ccccc6c45)c3)cc2)cc1 ; c1ccc(-c2ccc(-c3ccccc3N(c3ccc(-c4ccc5ccccc5c4-c4ccccc4)cc3)c3cccc4oc5ccccc5c34)cc2)cc1 ; c1ccc(-c2c(-c3ccc(N(c4ccc(-c5cccc6ccccc56)cc4)c4cccc5oc6ccccc6c45)cc3)ccc3ccccc23)cc1 ; c1ccc(-c2c(-c3ccc(N(c4ccc(-c5ccc6ccccc6c5)cc4)c4cccc5oc6ccccc6c45)cc3)ccc3ccccc23)cc1 ; c1ccc(-c2c(-c3ccc(N(c4ccc(-c5cc6ccccc6c6ccccc56)cc4)c4cccc5oc6ccccc6c45)cc3)ccc3ccccc23)cc1] 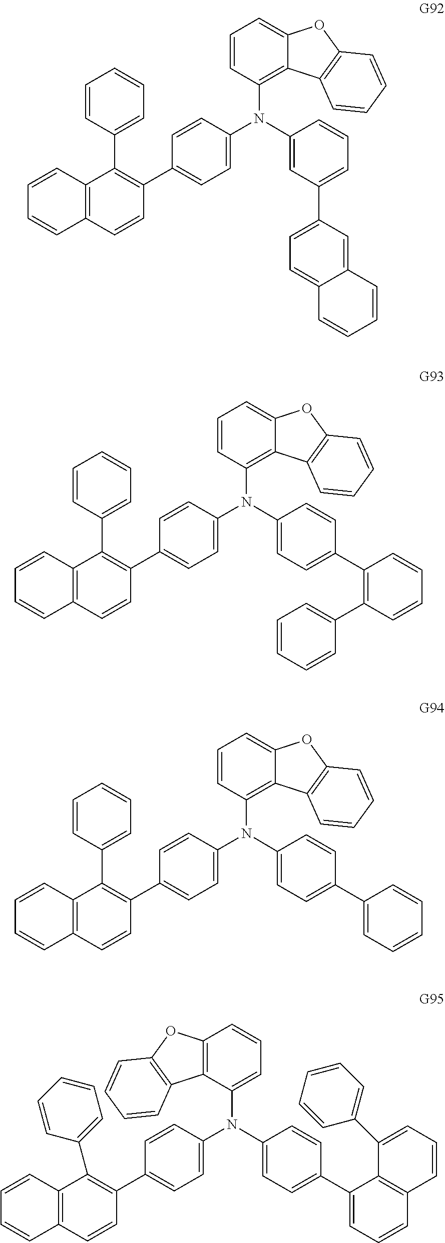 [SMILES: c1ccc(-c2c(-c3ccc(N(c4cccc(-c5ccc6ccccc6c5)c4)c4cccc5oc6ccccc6c45)cc3)ccc3ccccc23)cc1 ; c1ccc(-c2ccccc2-c2ccc(N(c3ccc(-c4ccc5ccccc5c4-c4ccccc4)cc3)c3cccc4oc5ccccc5c34)cc2)cc1 ; c1ccc(-c2ccc(N(c3ccc(-c4ccc5ccccc5c4-c4ccccc4)cc3)c3cccc4oc5ccccc5c34)cc2)cc1 ; c1ccc(-c2c(-c3ccc(N(c4ccc(-c5cccc6cccc(-c7ccccc7)c56)cc4)c4cccc5oc6ccccc6c45)cc3)ccc3ccccc23)cc1] 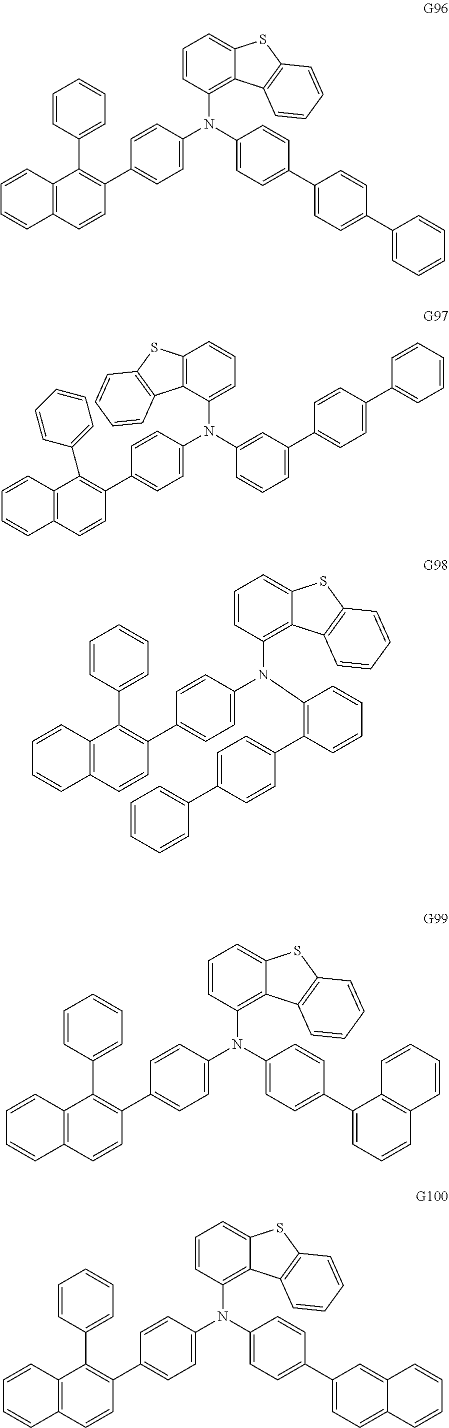 [SMILES: c1ccc(-c2ccc(-c3ccc(N(c4ccc(-c5ccc6ccccc6c5-c5ccccc5)cc4)c4cccc5sc6ccccc6c45)cc3)cc2)cc1 ; c1ccc(-c2ccc(-c3cccc(N(c4ccc(-c5ccc6ccccc6c5-c5ccccc5)cc4)c4cccc5sc6ccccc6c45)c3)cc2)cc1 ; c1ccc(-c2ccc(-c3ccccc3N(c3ccc(-c4ccc5ccccc5c4-c4ccccc4)cc3)c3cccc4sc5ccccc5c34)cc2)cc1 ; c1ccc(-c2c(-c3ccc(N(c4ccc(-c5cccc6ccccc56)cc4)c4cccc5sc6ccccc6c45)cc3)ccc3ccccc23)cc1 ; c1ccc(-c2c(-c3ccc(N(c4ccc(-c5ccc6ccccc6c5)cc4)c4cccc5sc6ccccc6c45)cc3)ccc3ccccc23)cc1] 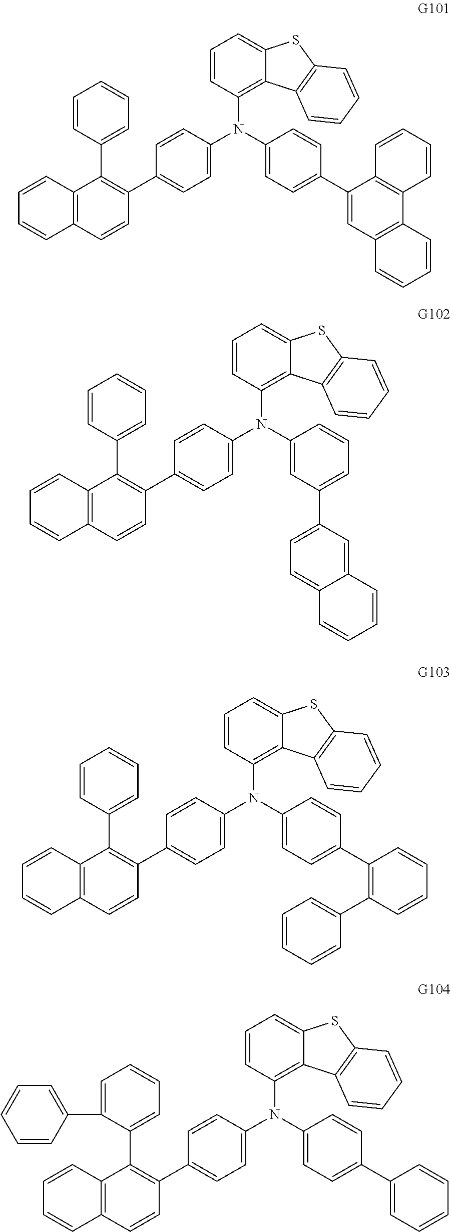 [SMILES: c1ccc(-c2c(-c3ccc(N(c4ccc(-c5cc6ccccc6c6ccccc56)cc4)c4cccc5sc6ccccc6c45)cc3)ccc3ccccc23)cc1 ; c1ccc(-c2c(-c3ccc(N(c4cccc(-c5ccc6ccccc6c5)c4)c4cccc5sc6ccccc6c45)cc3)ccc3ccccc23)cc1 ; c1ccc(-c2ccccc2-c2ccc(N(c3ccc(-c4ccc5ccccc5c4-c4ccccc4)cc3)c3cccc4sc5ccccc5c34)cc2)cc1 ; c1ccc(-c2ccc(N(c3ccc(-c4ccc5ccccc5c4-c4ccccc4-c4ccccc4)cc3)c3cccc4sc5ccccc5c34)cc2)cc1] 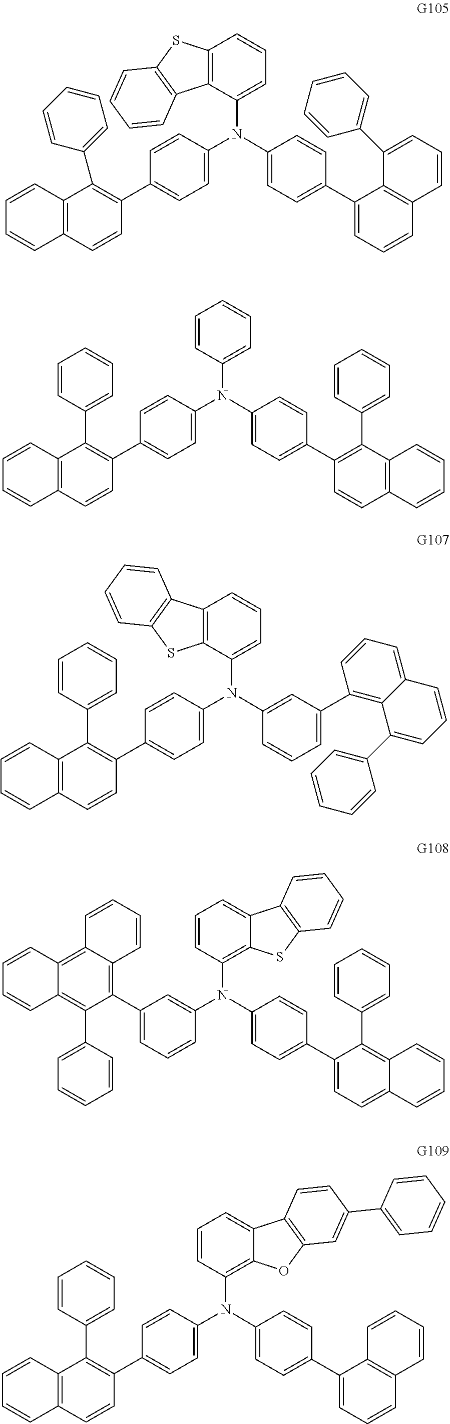 [SMILES: c1ccc(-c2c(-c3ccc(N(c4ccc(-c5cccc6cccc(-c7ccccc7)c56)cc4)c4cccc5sc6ccccc6c45)cc3)ccc3ccccc23)cc1 ; c1ccc(-c2c(-c3ccc(N(c4ccccc4)c4ccc(-c5ccc6ccccc6c5-c5ccccc5)cc4)cc3)ccc3ccccc23)cc1 ; c1ccc(-c2c(-c3ccc(N(c4cccc(-c5cccc6cccc(-c7ccccc7)c56)c4)c4cccc5c4sc4ccccc45)cc3)ccc3ccccc23)cc1 ; c1ccc(-c2c(-c3ccc(N(c4cccc(-c5c(-c6ccccc6)c6ccccc6c6ccccc56)c4)c4cccc5c4sc4ccccc45)cc3)ccc3ccccc23)cc1 ; c1ccc(-c2ccc3c(c2)oc2c(N(c4ccc(-c5ccc6ccccc6c5-c5ccccc5)cc4)c4ccc(-c5cccc6ccccc56)cc4)cccc23)cc1] 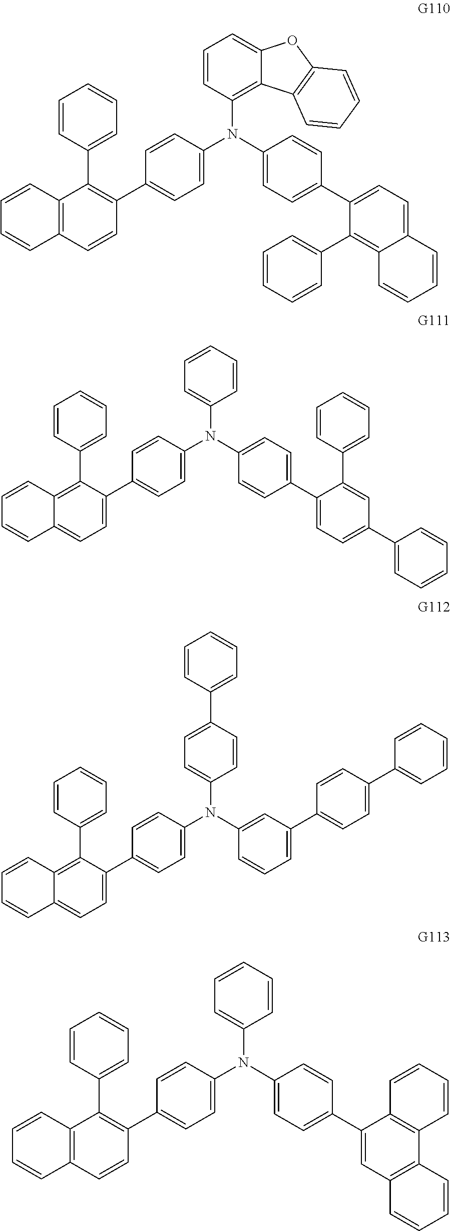 [SMILES: c1ccc(-c2c(-c3ccc(N(c4ccc(-c5ccc6ccccc6c5-c5ccccc5)cc4)c4cccc5oc6ccccc6c45)cc3)ccc3ccccc23)cc1 ; c1ccc(-c2ccc(-c3ccc(N(c4ccccc4)c4ccc(-c5ccc6ccccc6c5-c5ccccc5)cc4)cc3)c(-c3ccccc3)c2)cc1 ; c1ccc(-c2ccc(-c3cccc(N(c4ccc(-c5ccccc5)cc4)c4ccc(-c5ccc6ccccc6c5-c5ccccc5)cc4)c3)cc2)cc1 ; c1ccc(-c2c(-c3ccc(N(c4ccccc4)c4ccc(-c5cc6ccccc6c6ccccc56)cc4)cc3)ccc3ccccc23)cc1] 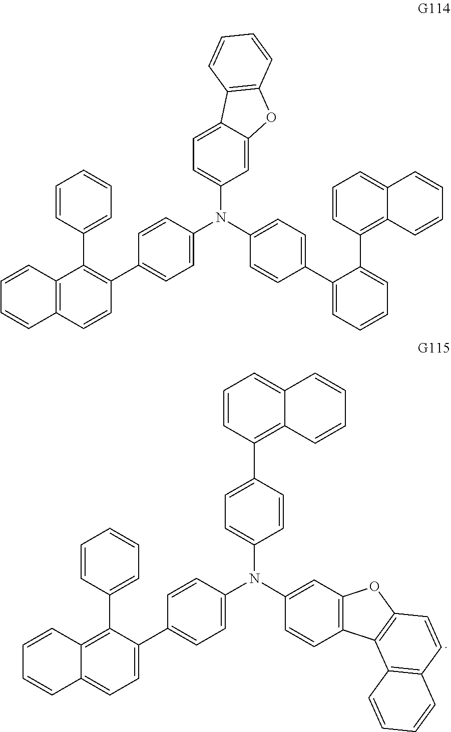 [SMILES: c1ccc(-c2c(-c3ccc(N(c4ccc(-c5ccccc5-c5cccc6ccccc56)cc4)c4ccc5c(c4)oc4ccccc45)cc3)ccc3ccccc23)cc1 ; c1ccc(-c2c(-c3ccc(N(c4ccc(-c5cccc6ccccc56)cc4)c4ccc5c(c4)oc4ccc6ccccc6c45)cc3)ccc3ccccc23)cc1]
